(19) United States
(12) Patent Application Publication
(10) Pub. No.: US 2026/0275333 A1
(43) Pub. Date: Sep. 17, 2026

(54) SPLICE SWITCHER ANTISENSE OLIGONUCLEOTIDES WITH MODIFIED BACKBONE CHEMISTRIES

(71) Applicant: QURALIS CORPORATION, Cambridge, MA (US)

(72) Inventors: SANDRA HINCKLEY, Cambridge, MA (US); DUNCAN BROWN, Berkeley, CA (US); DANIEL ELBAUM, Cambridge, MA (US); MARISA ELIZABETH KAMELGARN, Cambridge, MA (US)

(21) Appl. No.: 18/715,684

(22) PCT Filed: Dec. 2, 2022
(86) PCT No.: PCT/US2022/051740

§ 371 Date: Jun. 3, 2024

Related U.S. Application Data
(60) Provisional application No. 63/285,628, filed on Dec. 3, 2021.
(60) Provisional application No. 63/285,631, filed on Dec. 3, 2021.
(60) Provisional application No. 63/285,786, filed on Dec. 3, 2021.
(60) Provisional application No. 63/285,933, filed on Dec. 3, 2021.
(60) Provisional application No. 63/350,206, filed on Jun. 8, 2022.
(60) Provisional application No. 63/398,987, filed on Aug. 18, 2022.
(60) Provisional application No. 63/398,992, filed on Aug. 18, 2022.

(51) Int. Cl. C12N 15/113; A61K 31/712; A61K 31/7125; A61K 31/713; A61K 45/06; A61P 25/28; C07H 1/00; C07H 21/02

(52) U.S. Cl. CPC C12N 15/113; A61K 31/712; A61K 31/7125; A61K 31/713; A61K 45/06; A61P 25/28; C07H 1/00; C07H 21/02; C12N 2310/11; C12N 2310/315; C12N 2310/321; C12N 2310/322; C12N 2310/3231; C12N 2320/33

(57) ABSTRACT

Disclosed herein are oligonucleotides with one or more spacers or without a spacer. In various embodiments, STMN2, KCNQ2, UNC13A, or SMN2 oligonucleotides with spacer(s) reduce STMN2 transcripts with cryptic exons or reduce mis-spliced KCNQ2, UNC13A, or SMN2 transcripts and increase full length STMN2, KCNQ2, UNC13A, SMN transcripts, thereby imparting therapeutic efficacy against neurological diseases such as amyotrophic lateral sclerosis (ALS), frontotemporal dementia (FTD), Alzheimer's disease (AD) or spinal muscular atrophy (SMA).

### CROSS-REFERENCE TO RELATED APPLICATIONS

This application claims the benefit of and priority to U.S. Provisional Patent Application No. 63/285,933 filed Dec. 3, 2021, U.S. Provisional Patent Application No. 63/285,628 filed Dec. 3, 2021, U.S. Provisional Patent Application No. 63/285,786 filed Dec. 3, 2021, U.S. Provisional Patent Application No. 63/285,631 filed Dec. 3, 2021, U.S. Provisional Patent Application No. 63/350,206 filed Jun. 8, 2022, U.S. Provisional Patent Application No. 63/398,987 filed Aug. 18, 2022, and U.S. Provisional Patent Application No. 63/398,992 filed Aug. 18, 2022, the entire disclosure of each of which is hereby incorporated by reference in its entirety for all purposes.

### BACKGROUND

Antisense oligonucleotides are nucleic acid-based compounds that can be used to inhibit expression of certain genes that are linked to diseases. Although antisense oligonucleotides can be generally designed to hybridize with target genes, conventional antisense oligonucleotides often exhibit poor efficacy. Thus, there is a need to develop modified antisense oligonucleotides that exhibit improved performance and efficacy for preventing, ameliorating, and treating diseases, examples of which include neurological diseases.

Thus, there is a pressing need to identify compounds and/or compositions capable of preventing, ameliorating, and treating neurological diseases such as: amyotrophic lateral sclerosis (ALS), frontotemporal dementia (FTD), ALS with FTD, Alzheimer's disease (AD), Parkinson's disease (PD), Huntington's disease, progressive supranuclear palsy (PSP), brain trauma, spinal cord injury, corticobasal degeneration (CBD), nerve injuries (e.g., brachial plexus injuries), neuropathies (e.g., chemotherapy induced neuropathy), TDP43 proteinopathies (e.g., chronic traumatic encephalopathy, Perry Syndrome, Dementia with Lewy body in association with Alzheimer's disease, Parkinson's disease with or without dementia, and Limbic-predominant age-related TDP-43 encephalopathy (LATE)), Cerebral Age-Related TDP-43 With Sclerosis (CARTS), facial onset sensory and motor neuronopathy, Guam Parkinson-dementia complex, multisystem proteinopathy, CTE, spinal muscular atrophy (SMA), and synaptic diseases like autism.

### SUMMARY

Disclosed herein is a compound comprising a splice-switching oligonucleotide, and wherein the splice-switching oligonucleotide comprises a spacer. Additionally disclosed herein is a splice-switching oligonucleotide, wherein the splice-switching oligonucleotide comprises a spacer.

Additionally disclosed herein is a compound comprising a splice-switching oligonucleotide comprising a sequence that is at least 85% complementary to an equal length portion of a sequence from a transcript whose mis-splicing leads to a neurological disease or a transcript whose splicing is capable of being modulated to treat a neurological disease, wherein at least one (i.e., one or more) nucleoside linkage of the oligonucleotide is a non-natural linkage.

Additionally disclosed herein is a compound comprising a splice-switching oligonucleotide comprising a sequence that is at least 85% complementary to an equal length portion of a sequence from a transcript regulated by TDP-43, wherein at least one (i.e., one or more) nucleoside linkage of the oligonucleotide is a non-natural linkage.

In various embodiments, the splice-switching oligonucleotide comprises a sequence that is at least 85% complementary to an equal length portion of a sequence from a transcript of any one of SEQ ID NO: 1339 or SEQ ID NO: 1341, SEQ ID NO: 3032-3043, SEQ ID NO: 9587-9595, or SEQ ID NO: 9698-9707.

In various embodiments, the splice-switching oligonucleotide comprises a sequence that is at least 85% complementary to an equal length portion of any one of SEQ ID NO: 1339 or SEQ ID NO: 1341, SEQ ID NO: 3032-3043, SEQ ID NO: 9587-9595, or SEQ ID NO: 9698-9707.

Additionally disclosed herein is a compound comprising a modified splice-switching oligonucleotide comprising a sequence that is at least 85% complementary to an equal length portion of a sequence from a transcript of any one STMN2, KCNQ2, UNC13A, or SMN2, wherein at least one (i.e., one or more) nucleoside linkage of the oligonucleotide is a non-natural linkage.

In various embodiments, the oligonucleotide comprises a sequence that is at least 85% complementary to an equal length portion of any one of SEQ ID NO: 1339 or SEQ ID NO: 1341, SEQ ID NO: 3032-3043, SEQ ID NO: 9587-9595, or SEQ ID NO: 9698-9707, a sequence having 90% identity thereof, or to a 15 to 50 contiguous nucleobase portion thereof.

Additionally disclosed herein is a compound comprising a splice-switching oligonucleotide, wherein the splice-switching oligonucleotide comprises a spacer.

In various embodiments, the splice-switching oligonucleotide comprises a sequence that is at least 85% complementary to an equal length portion of a sequence from a transcript whose mis-splicing leads to a neurological disease or a transcript whose splicing is capable of being modulated to treat a neurological disease.

In various embodiments, the splice-switching oligonucleotide comprises a sequence that is at least 85% complementary to an equal length portion of a sequence from a transcript regulated by TDP-43, wherein at least one (i.e., one or more) nucleoside linkage of the oligonucleotide is a non-natural linkage, and further wherein the oligonucleotide comprises a spacer.

In various embodiments, the oligonucleotide comprises a sequence that is at least 85% complementary to an equal length portion of a sequence from a transcript of any one STMN2, KCNQ2, UNC13A, or SMN2. In various embodiments, the modified oligonucleotide comprises a sequence that is at least 85% complementary to an equal length portion of any one of SEQ ID NO: 1339 or SEQ ID NO: 1341, SEQ ID NO: 3032-3043, SEQ ID NO: 9587-9595, or SEQ ID NO: 9698-9707.

Additionally disclosed herein is a compound comprising a modified oligonucleotide comprising a sequence that is at least 85% complementary to an equal length portion of a sequence from a transcript of any one STMN2, KCNQ2, UNC13A, or SMN2, wherein at least one (i.e., one or more) nucleoside linkage of the oligonucleotide is a non-natural linkage, and further wherein the oligonucleotide comprises a spacer.

In various embodiments, the modified oligonucleotide comprises a sequence that is at least 85% complementary to an equal length portion of any one of SEQ ID NO: 1339 or SEQ ID NO: 1341, SEQ ID NO: 3032-3043, SEQ ID NO: 9587-9595, or SEQ ID NO: 9698-9707, a sequence having 90% identity thereof, or to a 15 to 50 contiguous nucleobase portion thereof.

Additionally disclosed herein is a splice-switching oligonucleotide comprising a sequence that is at least 85% complementary to an equal length portion of a sequence from a transcript whose mis-splicing leads to a neurological disease or a transcript whose splicing is capable of being modulated to treat a neurological disease, wherein at least one (i.e., one or more) nucleoside linkage of the oligonucleotide is a non-natural linkage.

Additionally disclosed herein is a splice-switching oligonucleotide comprising a sequence that is at least 85% complementary to an equal length portion of a sequence from a transcript regulated by TDP-43, wherein at least one (i.e., one or more) nucleoside linkage of the oligonucleotide is a non-natural linkage.

In various embodiments, the oligonucleotide comprises a sequence that is at least 85% complementary to an equal length portion of a sequence from a transcript of any one of STMN2, KCNQ2, UNC13A, or SMN2. In various embodiments, the modified oligonucleotide comprises a sequence that is at least 85% complementary to an equal length portion of any one of SEQ ID NO: 1339 or SEQ ID NO: 1341, SEQ ID NO: 3032-3043, SEQ ID NO: 9587-9595, or SEQ ID NO: 9698-9707.

Additionally disclosed herein is a splice-switching oligonucleotide comprising a sequence that is at least 85% complementary to an equal length portion of a sequence from a transcript of any one of STMN2, KCNQ2, UNC13A, or SMN2, wherein at least one (i.e., one or more) nucleoside linkage of the oligonucleotide is a non-natural linkage.

In various embodiments, the oligonucleotide comprises a sequence that is at least 85% complementary to an equal length portion of any one of SEQ ID NO: 1339 or SEQ ID NO: 1341, SEQ ID NO: 3032-3043, SEQ ID NO: 9587-9595, or SEQ ID NO: 9698-9707.

Additionally disclosed herein is a splice-switching oligonucleotide comprising a sequence that is at least 85% complementary to an equal length portion of any one of SEQ ID NO: 1339 or SEQ ID NO: 1341, SEQ ID NO: 3032-3043, SEQ ID NO: 9587-9595, or SEQ ID NO: 9698-9707, wherein at least one (i.e., one or more) nucleoside linkage of the oligonucleotide is a non-natural linkage. In various embodiments, the oligonucleotide further comprises a spacer.

In various embodiments, the oligonucleotide comprises a sequence that is at least 85% complementary to an equal length portion of a sequence from a transcript whose mis-splicing leads to a neurological disease or a transcript whose splicing is capable of being modulated to treat a neurological disease.

In various embodiments, the oligonucleotide comprises a sequence that is at least 85% complementary to an equal length portion of a sequence from a transcript regulated by TDP-43, wherein at least one (i.e., one or more) nucleoside linkage of the oligonucleotide is a non-natural linkage, and further wherein the oligonucleotide comprises a spacer.

In various embodiments, the oligonucleotide comprises a sequence that is at least 85% complementary to an equal length portion of a sequence from a transcript of any one STMN2, KCNQ2, UNC13A, or SMN2. In various embodiments, the modified oligonucleotide comprises a sequence that is at least 85% complementary to an equal length portion of any one of SEQ ID NO: 1339 or SEQ ID NO: 1341, SEQ ID NO: 3032-3043, SEQ ID NO: 9587-9595, or SEQ ID NO: 9698-9707.

Additionally disclosed herein is a splice-switching oligonucleotide comprising a sequence that is at least 85% complementary to an equal length portion of a sequence from a transcript of any one STMN2, KCNQ2, UNC13A, or SMN2, wherein at least one (i.e., one or more) nucleoside linkage of the oligonucleotide is a non-natural linkage, and further wherein the oligonucleotide comprises a spacer.

In various embodiments, the oligonucleotide comprises a sequence that is at least 85% complementary to an equal length portion of any one of SEQ ID NO: 1339 or SEQ ID NO: 1341, SEQ ID NO: 3032-3043, SEQ ID NO: 9587-9595, or SEQ ID NO: 9698-9707.

Additionally disclosed herein is an oligonucleotide comprising a sequence that is at least 85% complementary to an equal length portion of any one of SEQ ID NO: 1339 or SEQ ID NO: 1341, SEQ ID NO: 3032-3043, SEQ ID NO: 9587-9595, or SEQ ID NO: 9698-9707, a sequence having 90% identity thereof, or to a 15 to 50 contiguous nucleobase portion thereof, wherein at least one (i.e., one or more) nucleoside linkage of the oligonucleotide is a non-natural linkage, and further wherein the oligonucleotide comprises a spacer.

In various embodiments, the oligonucleotide comprises a segment with at most 11 linked nucleosides. In various embodiments, the oligonucleotide comprises a segment with at most 10, 9, or 8 linked nucleosides. In various embodiments, the oligonucleotide comprises a segment with at most 7 linked nucleosides. In various embodiments, the oligonucleotide comprises a segment with at most 6, 5, 4, 3, or 2 linked nucleosides. In various embodiments, every segment of the oligonucleotide comprises at most 7 linked nucleosides.

In various embodiments, the oligonucleotide comprises a sequence that shares at least 85% identity with an equal length portion of any one of SEQ ID NOs: 1-466, SEQ ID NOs: 893-1338, SEQ ID NOs: 1342-1366, SEQ ID NOs: 1392-1664, or SEQ ID NOs: 10655-10669, SEQ ID NOs: 1676-1851, SEQ ID NOs: 2028-2529, SEQ ID NOs: 3046-3221, SEQ ID NOs: 3398-3899, and SEQ ID NOs: 4402-4530, SEQ ID NOs: 4531-5794, SEQ ID NOs: 7059-8322, SEQ ID NOs: 9596-9603, SEQ ID NOs: 9710-10141, SEQ ID NOs: 10574-10651, SEQ ID NOs: 10670-10779, SEQ ID NOs: 10783-10808, and SEQ ID NOs: 10814-10820.

In various embodiments, the oligonucleotide comprises a sequence that shares at least 90% identity with an equal length portion of any one of SEQ ID NOs: 1-466, SEQ ID NOs: 893-1338, SEQ ID NOs: 1342-1366, SEQ ID NOs: 1392-1664, or SEQ ID NOs: 10655-10669, SEQ ID NOs: 1676-1851, SEQ ID NOs: 2028-2529, SEQ ID NOs: 3046-3221, SEQ ID NOs: 3398-3899, and SEQ ID NOs: 4402-4530, SEQ ID NOs: 4531-5794, SEQ ID NOs: 7059-8322, SEQ ID NOs: 9596-9603, SEQ ID NOs: 9710-10141, SEQ ID NOs: 10574-10651, SEQ ID NOs: 10670-10779, SEQ ID NOs: 10783-10808, and SEQ ID NOs: 10814-10820.

In various embodiments, the oligonucleotide comprises a sequence that shares at least 95% identity with an equal length portion of any one of SEQ ID NOs: 1-466, SEQ ID NOs: 893-1338, SEQ ID NOs: 1342-1366, SEQ ID NOs: 1392-1664, or SEQ ID NOs: 10655-10669, SEQ ID NOs: 1676-1851, SEQ ID NOs: 2028-2529, SEQ ID NOs: 3046-3221, SEQ ID NOs: 3398-3899, and SEQ ID NOs: 4402-4530, SEQ ID NOs: 4531-5794, SEQ ID NOs: 7059-8322, SEQ ID NOs: 9596-9603, SEQ ID NOs: 9710-10141, SEQ ID NOs: 10574-10651, SEQ ID NOs: 10670-10779, SEQ ID NOs: 10783-10808, and SEQ ID NOs: 10814-10820.

In various embodiments, the oligonucleotide comprises a sequence that shares 100% identity with an equal length portion of any one of SEQ ID NOs: 1-466, SEQ ID NOs: 893-1338, SEQ ID NOs: 1342-1366, SEQ ID NOs: 1392-1664, or SEQ ID NOs: 10655-10669, SEQ ID NOs: 1676-1851, SEQ ID NOs: 2028-2529, SEQ ID NOs: 3046-3221, SEQ ID NOs: 3398-3899, and SEQ ID NOs: 4402-4530, SEQ ID NOs: 4531-5794, SEQ ID NOs: 7059-8322, SEQ ID NOs: 9596-9603, SEQ ID NOs: 9710-10141, SEQ ID NOs: 10574-10651, SEQ ID NOs: 10670-10779, SEQ ID NOs: 10783-10808, and SEQ ID NOs: 10814-10820.

In various embodiments, the oligonucleotide comprises a segment with at most 11 linked nucleosides or at most 7 linked nucleosides, and wherein the oligonucleotide comprises a sequence that shares at least 85% identity with an equal length portion of any one of SEQ ID NOs: 1-466, SEQ ID NOs: 893-1338, SEQ ID NOs: 1342-1366, SEQ ID NOs: 1392-1664, or SEQ ID NOs: 10655-10669, SEQ ID NOs: 1676-1851, SEQ ID NOs: 2028-2529, SEQ ID NOs: 3046-3221, SEQ ID NOs: 3398-3899, and SEQ ID NOs: 4402-4530, SEQ ID NOs: 4531-5794, SEQ ID NOs: 7059-8322, SEQ ID NOs: 9596-9603, SEQ ID NOs: 9710-10141, SEQ ID NOs: 10574-10651, SEQ ID NOs: 10670-10779, SEQ ID NOs: 10783-10808, and SEQ ID NOs: 10814-10820. In various embodiments, the oligonucleotide comprises a segment with at most 6, 5, 4, 3, or 2 linked nucleosides, and wherein the oligonucleotide comprises a sequence that shares at least 85% identity with an equal length portion of any one of SEQ ID NOs: 1-466, SEQ ID NOs: 893-1338, SEQ ID NOs: 1342-1366, SEQ ID NOs: 1392-1664, or SEQ ID NOs: 10655-10669, SEQ ID NOs: 1676-1851, SEQ ID NOs: 2028-2529, SEQ ID NOs: 3046-3221, SEQ ID NOs: 3398-3899, and SEQ ID NOs: 4402-4530, SEQ ID NOs: 4531-5794, SEQ ID NOs: 7059-8322, SEQ ID NOs: 9596-9603, SEQ ID NOs: 9710-10141, SEQ ID NOs: 10574-10651, SEQ ID NOs: 10670-10779, SEQ ID NOs: 10783-10808, and SEQ ID NOs: 10814-10820. In various embodiments, the oligonucleotide comprises a segment with at most 6, 5, 4, 3, or 2 linked nucleosides, and wherein the oligonucleotide comprises a sequence that shares at least 90% identity with an equal length portion of any one of SEQ ID NOs: 1-466, SEQ ID NOs: 893-1338, SEQ ID NOs: 1342-1366, SEQ ID NOs: 1392-1664, or SEQ ID NOs: 10655-10669, SEQ ID NOs: 1676-1851, SEQ ID NOs: 2028-2529, SEQ ID NOs: 3046-3221, SEQ ID NOs: 3398-3899, and SEQ ID NOs: 4402-4530, SEQ ID NOs: 4531-5794, SEQ ID NOs: 7059-8322, SEQ ID NOs: 9596-9603, SEQ ID NOs: 9710-10141, SEQ ID NOs: 10574-10651, SEQ ID NOs: 10670-10779, SEQ ID NOs: 10783-10808, and SEQ ID NOs: 10814-10820.

In various embodiments, the oligonucleotide is at least 18, at least 19, at least 20, at least 21, at least 22, at least 23, at least 24, or at least 25 oligonucleotide units in length. In various embodiments, the oligonucleotide is at least 19 oligonucleotide units in length.

In various embodiments, the spacer is a nucleoside-replacement group comprising a non-sugar substitute that is incapable of linking to a nucleotide base. In various embodiments, the spacer is located between positions 10 and 15 of the oligonucleotide. In various embodiments, the spacer is located between positions 7 and 11 of the oligonucleotide.

In various embodiments, the oligonucleotide further comprises a second spacer, wherein the second spacer is located between positions 14 and 22 of the oligonucleotide. In various embodiments, the spacer and the second spacer are separated by at least 5 nucleobases, at least 6 nucleobases, or at least 7 nucleobases in the oligonucleotide. In various embodiments, the spacer is located between positions 7 and 9 of the oligonucleotide, and wherein the second spacer is located between positions 15 and 18 of the oligonucleotide. In various embodiments, the spacer is located at position 8 of the oligonucleotide, and wherein the second spacer is located at position 16 of the oligonucleotide.

In various embodiments, the oligonucleotide further comprises a third spacer, wherein the third spacer is located between positions 21 and 24 of the oligonucleotide. In various embodiments, the spacer is located between positions 2 and 5 of the oligonucleotide.

In various embodiments, the oligonucleotide further comprises a second spacer, wherein the second spacer is located between positions 8 and 12 of the oligonucleotide. In various embodiments, the oligonucleotide further comprises a third spacer, wherein the third spacer is located between positions 18 and 22 of the oligonucleotide. In various embodiments, the oligonucleotide further comprises a second spacer and a third spacer, wherein the three spacers are located at positions in the oligonucleotide such that each segment of the oligonucleotide has at most 7 linked nucleosides.

In various embodiments, at least two of the three spacers are adjacent to a guanine nucleobase. In various embodiments, each of the at least two of the three spacers immediately precede a guanine nucleobase. In various embodiments, each of the first, second or third spacers is a nucleoside-replacement group comprising a non-sugar substitute wherein the non-sugar substitute does not contain a ketone, aldehyde, ketal, hemiketal, acetal, hemiacetal, aminal or hemiaminal moiety and is incapable of forming a covalent bond with a nucleotide base.

In various embodiments, each of the first, second or third spacers is independently represented by Formula (X), wherein:

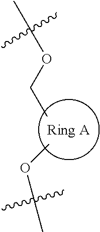

Ring A is an optionally substituted 4-8 member monocyclic cycloalkyl group or a 4-8 member monocyclic heterocyclyl group, wherein the heterocyclyl group contains 1 or 2 heteroatoms selected from O, S and N, provided that A is not capable of forming a covalent bond to a nucleobase; and the

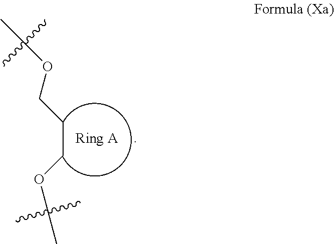 [SMILES: *OCCCO*]

symbol represents the point of connection to an internucleoside linkage.

In various embodiments, each of the first, second or third spacers is independently represented by Formula (Xa), wherein:

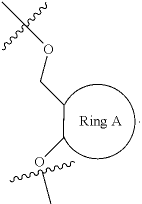

In various embodiments, ring A is an optionally substituted 4-8 member monocyclic cycloalkyl group selected from cyclobutyl, cyclopentyl, cyclohexyl, cycloheptyl and cyclooctyl; or a 4-8 member monocyclic heterocyclyl group, selected from oxetanyl, tetrahydrofuranyl, tetrahydropyranyl, 1,4-dioxanyl, pyrrolidinyl, piperidinyl, piperazinyl, morpholinyl and azepanyl.

In various embodiments, ring A is tetrahydrofuranyl. In various embodiments, ring A is tetrahydropyranyl.

In various embodiments, each of the first, second or third spacers is independently represented by Formula I, wherein:

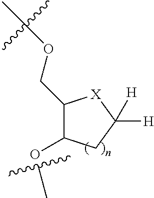

X is selected from —CH₂— and —O—; and
n is 0, 1, 2 or 3.

In various embodiments, each of the first, second or third spacers is independently represented by Formula I′, wherein:

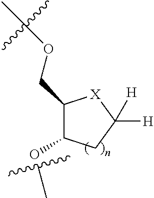

X is selected from —CH₂— and —O—; and
n is 0, 1, 2 or 3.

In various embodiments, each of the first, second or third spacers is independently represented by Formula (Ia), wherein:

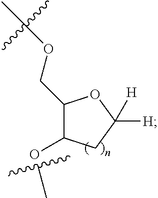

and
n is 0, 1, 2 or 3.

In various embodiments, each of the first, second or third spacers is independently represented by Formula (Ia′), wherein:

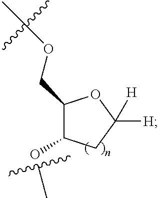

and
n is 0, 1, 2 or 3.

In various embodiments, each of the first, second or third spacers is independently represented by Formula II, wherein:

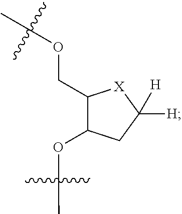

and
X is selected from —CH₂— and —O—.

In various embodiments, each of the first, second or third spacers is independently represented by Formula II′, wherein:

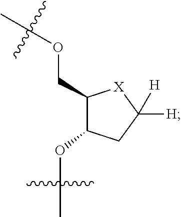

and
X is selected from —CH₂— and —O—.

In various embodiments, each of the first, second or third spacers is independently represented by Formula (Iia), wherein:

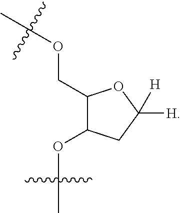

In various embodiments, each of the first, second or third spacers is independently represented by Formula (Iia′), wherein:

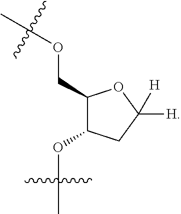

In some embodiments, the spacer is represented by Formula (IIi), wherein:

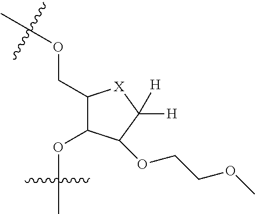

X is selected from —CH₂— and —O—.

In some embodiments, the spacer is represented by Formula (IIi′), wherein:

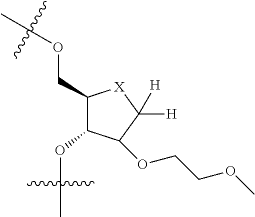

X is selected from —CH₂— and —O.

In some embodiments, the spacer is represented by Formula (IIib), wherein:

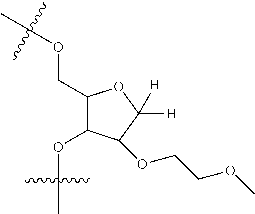 [SMILES: *OCC1OCC(OCCOC)C1O*]

In some embodiments, the spacer is represented by Formula (Iiib′), wherein:

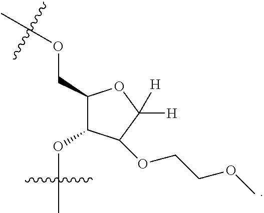 [SMILES: *OC[C@H]1OCC(OCCOC)[C@@H]1O*]

In various embodiments, each of the first, second or third spacers is independently represented by Formula III, wherein:

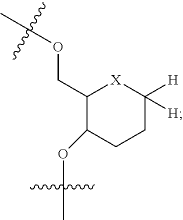

and
X is selected from —CH₂— and —O—.

In various embodiments, each of the first, second or third spacers is independently represented by Formula III′, wherein:

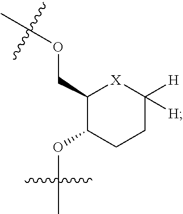

and
X is selected from —CH₂— and —O—.

In various embodiments, each of the first, second or third spacers is independently represented by Formula (IIIa), wherein:

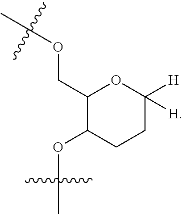

In various embodiments, each of the first, second or third spacers is independently represented by Formula (IIIa′), wherein:

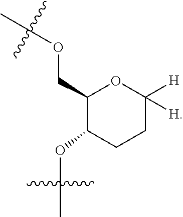

In various embodiments, the oligonucleotide comprising the spacer has a GC content of at least 10%. In various embodiments, the oligonucleotide comprising the spacer has a GC content of at least 20%. In various embodiments, the oligonucleotide comprising the spacer has a GC content of at least 25%. In various embodiments, the oligonucleotide comprising the spacer has a GC content of at least 30%. In various embodiments, the oligonucleotide comprising the spacer has a GC content of at least 40%. In various embodiments, the oligonucleotide comprising the spacer has a GC content of at least 50%. In various embodiments, the oligonucleotide is between 12 and 40 oligonucleotide units in length.

In various embodiments, at least one (i.e., one or more) nucleoside linkage of the oligonucleotide is independently selected from the group consisting of a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

In various embodiments, one or more nucleoside linkages that link a base at position 3 or position 4 of the oligonucleotide are phosphodiester linkages. In various embodiments, only one nucleoside linkage that links a base at position 3 or position 4 of the oligonucleotide is a phosphodiester linkage. In various embodiments, nucleoside linkages that link bases at both position 3 and position 4 of the oligonucleotide are phosphodiester linkages. In various embodiments, one or more bases immediately preceding a spacer in the oligonucleotide are linked through phosphodiester bonds. In various embodiments, only the base immediately preceding the spacer in the oligonucleotide is linked to the spacer through a phosphodiester bond. In various embodiments, the base immediately preceding the spacer in the oligonucleotide is further linked to a further preceding base through a phosphodiester bond.

In various embodiments, the oligonucleotide comprises a second spacer, wherein a base immediately preceding the second spacer is linked to a further preceding base through a phosphodiester bond. In various embodiments, one or more bases immediately succeeding a spacer in the oligonucleotide are linked through phosphodiester bonds. In various embodiments, only the base immediately succeeding the spacer in the oligonucleotide is linked to the spacer through a phosphodiester bond. In various embodiments, two bases immediately preceding the spacer in the oligonucleotide are linked through phosphodiester bonds.

In various embodiments, one or more bases immediately preceding a spacer in the oligonucleotide are linked through phosphodiester bonds and wherein one or more bases immediately succeeding the spacer in the oligonucleotide are linked through phosphodiester bonds. In various embodiments, one base immediately preceding the spacer and one base immediately succeeding the spacer are linked through phosphodiester bonds.

In various embodiments, the oligonucleotide includes a second spacer, and wherein one or more bases immediately preceding the second spacer in the oligonucleotide are linked through phosphodiester bonds and wherein one or more bases immediately succeeding the second spacer in the oligonucleotide are linked through phosphodiester bonds. In various embodiments, one base immediately preceding the second spacer and one base immediately succeeding the second spacer are linked through phosphodiester bonds.

In various embodiments, the oligonucleotide comprises a range of bases that are linked through phosphodiester bonds, the range of bases comprising at least two bases. In various embodiments, the oligonucleotide comprises a range of bases that are linked through phosphodiester bonds, the range of bases comprising at least five bases. In various embodiments, the oligonucleotide comprises two or more spacers, and wherein the range of bases are positioned between the at least two spacers.

In various embodiments, one or more internucleoside linkage of the oligonucleotide is a modified internucleoside linkage. In various embodiments, the modified internucleoside linkage of the oligonucleotide is a phosphorothioate linkage. In various embodiments, all internucleoside linkages of the oligonucleotide are phosphorothioate linkages. In various embodiments, the phosphorothioate linkage is in one of a Rp configuration or a Sp configuration.

In various embodiments, the oligonucleotide comprises at least one modified sugar moiety. In various embodiments, the modified sugar moiety is one of a 2′-OMe modified sugar moiety, bicyclic sugar moiety, 2′-O-(2-methoxyethyl) (2′-MOE), 2′-deoxy-2′-fluoro nucleoside, 2′-fluoro-β-D-arabinonucleoside, locked nucleic acid (LNA), constrained ethyl 2′-4′-bridged nucleic acid (cEt), S-cEt, tcDNA, hexitol nucleic acids (HNA), and tricyclic analog (e.g., tcDNA).

In various embodiments, the oligonucleotide exhibits at least a 30%, 40%, 50%, 60%, 70%, 80%, or 90% increase of full length STMN2, KCNQ2, UNC13A, or SMN2 protein. In various embodiments, the oligonucleotide exhibits at least a 100% increase of full length protein. In various embodiments, the oligonucleotide exhibits at least a 200% increase of full length protein. In various embodiments, the oligonucleotide exhibits at least a 300% increase of full length protein. In various embodiments, the oligonucleotide exhibits at least a 400% increase of full length protein.

In various embodiments, increase of the full length protein is measured in comparison to a reduced level of full length protein achieved using a TDP43 antisense oligonucleotide. In various embodiments, the oligonucleotide exhibits at least a 10%, 20%, 30%, 40%, 50%, 60%, 70%, 80%, 90%, or 100% rescue of full length protein. In various embodiments, the oligonucleotide exhibits at least a 50%, 60%, 70%, 80%, or 90% reduction of a mis-spliced transcript.

Additionally disclosed herein is a method of treating a neurological disease and/or a neuropathy in a patient in need thereof, the method comprising administering to the patient a compound or an oligonucleotide as disclosed herein.

In various embodiments, the neurological disease is selected from the group consisting of amyotrophic lateral sclerosis (ALS), frontotemporal dementia (FTD), ALS with FTD, Alzheimer's disease (AD), Parkinson's disease (PD), Parkinson's Disease with dementia, dementia with lewy bodies, synucleinopathies, Huntington's disease, Brachial plexus injuries, peripheral nerve injuries, progressive supranuclear palsy (PSP), brain trauma, spinal cord injury, tuberous sclerosis complex, Pick's Disease, tauopathies, primary age-related tauopathy, Down Syndrome, epilepsy/seizure disorder, depression, traumatic brain injury (TBI), chronic traumatic encephalopathy (CTE), HIV-associated neurocognitive disorders (HAND), multisystem atrophy, amnestic mild cognitive impairment, corticobasal degeneration (CBD) and/or neuropathies such a chemotherapy induced neuropathy, Spinocerebellar ataxia (SCA), SCA type 2, Spinal Muscular Atrophy (SMA), Parkinsonism, Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), Limbic-predominant age-related TDP-43 encephalopathy (LATE)), Cerebral Age-Related TDP-43 With Sclerosis (CARTS), Gaucher's disease, and facial onset sensory and motor neuronopathy, Guam Parkinson-dementia complex, multisystem proteinopathy, Perry disease, and synaptic diseases like autism. In various embodiments, the neurological disease is ALS. In various embodiments, the neurological disease is FTD. In various embodiments, the neurological disease is ALS with FTD. In various embodiments, the neurological disease is AD. In various embodiments, the neurological disease is PD. In various embodiments, the neurological disease is spinal muscular atrophy (SMA). In various embodiments, the neuropathy is chemotherapy induced neuropathy.

Additionally disclosed herein is a method of restoring axonal outgrowth and/or regeneration of a neuron, the method comprising exposing the neuron to a compound or an oligonucleotide as disclosed herein.

Additionally disclosed herein is a method of increasing, promoting, stabilizing, or maintaining any one of STMN2, KCNQ2, UNC13A, or SMN2 expression and/or function in a neuron, the method comprising exposing the cell to a compound or an oligonucleotide as disclosed herein. In various embodiments, the neuron is a motor neuron. In various embodiments, the neuron is a spinal cord neuron. In various embodiments, the neuron is a neuron of a patient in need of treatment of a neurological disease and/or a neuropathy. In various embodiments, the neuropathy is chemotherapy induced neuropathy. In various embodiments, the exposing is performed in vivo or ex vivo. In various embodiments, the exposing comprises administering the oligonucleotide to a patient in need thereof.

In various embodiments, the oligonucleotide is administered topically, parenterally, intrathecally, intrathalamically, intracisternally, orally, rectally, buccally, sublingually, vaginally, pulmonarily, intratracheally, intranasally, transdermally, intraduodenally, or intracerebroventricularly. In various embodiments, the oligonucleotide is administered orally. In various embodiments, a therapeutically effective amount of the oligonucleotide is administered intrathecally, intrathalamically, intracerebroventricularly, or intracisternally. In various embodiments, the patient is a human.

Additionally disclosed herein is a pharmaceutical composition comprising the oligonucleotide disclosed herein, or a pharmaceutically acceptable salt thereof, and a pharmaceutically acceptable excipient.

In various embodiments, the pharmaceutical composition is suitable for topical, intrathecal, intrathalamic, intracisternal, intracerebroventricular, parenteral, oral, pulmonary, intratracheal, intranasal, transdermal, rectal, buccal, sublingual, vaginal, or intraduodenal administration.

Additionally disclosed herein is a method of treating a neurological disease or a neuropathy in a patient in need thereof, the method comprising administering to a patient in need thereof a therapeutically effective amount of a pharmaceutical composition disclosed herein.

In various embodiments, the neurological disease is selected from the group consisting of amyotrophic lateral sclerosis (ALS), frontotemporal dementia (FTD), ALS with FTD, Alzheimer's disease (AD), Parkinson's disease (PD), Parkinson's Disease with dementia, dementia with lewy bodies, synucleinopathies, Huntington's disease, Brachial plexus injuries, peripheral nerve injuries, progressive supranuclear palsy (PSP), brain trauma, spinal cord injury, tuberous sclerosis complex, Pick's Disease, tauopathies, primary age-related tauopathy, Down Syndrome, epilepsy/seizure disorder, depression, traumatic brain injury (TBI), chronic traumatic encephalopathy (CTE), HIV-associated neurocognitive disorders (HAND), multisystem atrophy, amnestic mild cognitive impairment, corticobasal degeneration (CBD) and/or neuropathies such a chemotherapy induced neuropathy, Spinocerebellar ataxia (SCA), SCA type 2, Spinal Muscular Atrophy (SMA), Parkinsonism, Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), Limbic-predominant age-related TDP-43 encephalopathy (LATE)), Cerebral Age-Related TDP-43 With Sclerosis (CARTS), Gaucher's disease, and facial onset sensory and motor neuronopathy, Guam Parkinson-dementia complex, multisystem proteinopathy, Perry disease, and synaptic diseases like autism.

In various embodiments, the neurological disease is ALS. In various embodiments, the neurological disease is FTD. In various embodiments, the neurological disease is ALS with FTD. In various embodiments, the neurological disease is AD. In various embodiments, the neurological disease is PD. In various embodiments, the neurological disease is spinal muscular atrophy (SMA). In various embodiments, the neuropathy is chemotherapy induced neuropathy.

In various embodiments, the pharmaceutical composition is administered topically, parenterally, orally, pulmonarily, rectally, buccally, sublingually, vaginally, intratracheally, intranasally, intracisternally, intrathecally, intrathalamically, transdermally, intraduodenally, or intracerebroventricularly. In various embodiments, the pharmaceutical composition is administered intrathecally, intrathalamically, or intracisternally. In various embodiments, a therapeutically effective amount of the oligonucleotide is administered intrathecally, intrathalamically or intracisternally. In various embodiments, the patient is human.

Additionally disclosed herein is a method for treating a neurological disease in a subject in need thereof, the method comprising administering to the subject an oligonucleotide comprising a segment with at most 7 linked nucleosides, and wherein the oligonucleotide is at least 85% complementary to an equal length portion of a sequence from a transcript whose mis-splicing leads to a neurological disease or a transcript whose splicing is capable of being modulated to treat a neurological disease, or a pharmaceutically acceptable salt thereof;
wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or
wherein at least one (i.e., one or more) nucleoside is substituted with a component selected from the group consisting of a 2′-O-(2-methoxyethyl) nucleoside, a 2′-O-methyl nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA)
optionally, wherein the oligonucleotide further comprises a spacer.

Additionally disclosed herein is a method for treating a neurological disease in a subject in need thereof, the method comprising administering to the subject an oligonucleotide comprising a segment with at most 7 linked nucleosides, and wherein oligonucleotide is at least 85% complementary to a sequence from a transcript of any one STMN2, KCNQ2, UNC13A, or SMN2, or a pharmaceutically acceptable salt thereof;
wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or
wherein at least one (i.e., one or more) nucleoside is substituted with a component selected from the group consisting of a 2′-O-(2-methoxyethyl) nucleoside, a 2′-O-methyl nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA)
optionally, wherein the oligonucleotide further comprises a spacer.

Additionally disclosed herein is a method for treating a neurological disease in a subject in need thereof, the method comprising administering to the subject an oligonucleotide comprising a segment with at most 7 linked nucleosides, and wherein oligonucleotide shares at least 85% identity with any one of SEQ ID NOs: 1-466, SEQ ID NOs: 893-1338, SEQ ID NOs: 1342-1366, SEQ ID NOs: 1392-1664, or SEQ ID NOs: 10655-10669, SEQ ID NOs: 1676-1851, SEQ ID NOs: 2028-2529, SEQ ID NOs: 3046-3221, SEQ ID NOs: 3398-3899, and SEQ ID NOs: 4402-4530, SEQ ID NOs: 4531-5794, SEQ ID NOs: 7059-8322, SEQ ID NOs: 9596-9603, SEQ ID NOs: 9710-10141, SEQ ID NOs: 10574-10651, SEQ ID NOs: 10670-10779, SEQ ID NOs: 10783-10808, and SEQ ID NOs: 10814-10820, or a pharmaceutically acceptable salt thereof;
wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or
wherein at least one (i.e., one or more) nucleoside is substituted with a component selected from the group consisting of a 2′-O-(2-methoxyethyl) nucleoside, a 2′-O-methyl nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA)
optionally, wherein the oligonucleotide further comprises a spacer.

Additionally disclosed herein is a method for treating amyotrophic lateral sclerosis (ALS) in a subject in need thereof, the method comprising administering to the subject an oligonucleotide comprising a segment with at most 7 linked nucleosides, and wherein oligonucleotide shares at least 85% identity with any one of SEQ ID NOs: 1-466, SEQ ID NOs: 893-1338, SEQ ID NOs: 1342-1366, SEQ ID NOs: 1392-1664, or SEQ ID NOs: 10655-10669, SEQ ID NOs: 1676-1851, SEQ ID NOs: 2028-2529, SEQ ID NOs: 3046-3221, SEQ ID NOs: 3398-3899, and SEQ ID NOs: 4402-4530, or SEQ ID NOs: 4531-5794, SEQ ID NOs: 7059-8322, or SEQ ID NOs; 9596-9603, or a pharmaceutically acceptable salt thereof;
wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or
wherein at least one (i.e., one or more) nucleoside is substituted with a component selected from the group consisting of a 2′-O-(2-methoxyethyl) nucleoside, a 2′-O-methyl nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA)
optionally, wherein the oligonucleotide further comprises a spacer.

Additionally disclosed herein is a method for treating Alzheimer's Disease (AD) with frontotemporal dementia (FTD) in a subject in need thereof, the method comprising administering to the subject an oligonucleotide comprising a segment with at most 7 linked nucleosides, and wherein oligonucleotide shares at least 85% identity with any one of SEQ ID NOs: 1-466, SEQ ID NOs: 893-1338, SEQ ID NOs: 1342-1366, SEQ ID NOs: 1392-1664, or SEQ ID NOs: 10655-10669, SEQ ID NOs: 1676-1851, SEQ ID NOs: 2028-2529, SEQ ID NOs: 3046-3221, SEQ ID NOs: 3398-3899, and SEQ ID NOs: 4402-4530, or SEQ ID NOs: 4531-5794, SEQ ID NOs: 7059-8322, or SEQ ID NOs; 9596-9603, or a pharmaceutically acceptable salt thereof;
wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or
wherein at least one (i.e., one or more) nucleoside is substituted with a component selected from the group consisting of a 2′-O-(2-methoxyethyl) nucleoside, a 2′-O-methyl nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA), optionally, wherein the oligonucleotide further comprises a spacer

Additionally disclosed herein is a method for treating frontotemporal dementia (FTD) in a subject in need thereof, the method comprising administering to the subject an oligonucleotide comprising a segment with at most 7 linked nucleosides, and wherein oligonucleotide shares at least 85% identity with any one of SEQ ID NOs: 1-466, SEQ ID NOs: 893-1338, SEQ ID NOs: 1342-1366, SEQ ID NOs: 1392-1664, or SEQ ID NOs: 10655-10669, SEQ ID NOs: 1676-1851, SEQ ID NOs: 2028-2529, SEQ ID NOs: 3046-3221, SEQ ID NOs: 3398-3899, and SEQ ID NOs: 4402-4530, or SEQ ID NOs: 4531-5794, SEQ ID NOs: 7059-8322, or SEQ ID NOs; 9596-9603, or a pharmaceutically acceptable salt thereof;
wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or
wherein at least one (i.e., one or more) nucleoside is substituted with a component selected from the group consisting of a 2′-O-(2-methoxyethyl) nucleoside, a 2′-O-methyl nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA)
optionally, wherein the oligonucleotide further comprises a spacer.

Additionally disclosed herein is a method for treating amyotrophic lateral sclerosis (ALS) with frontotemporal dementia (FTD) in a subject in need thereof, the method comprising administering to the subject an oligonucleotide comprising a segment with at most 7 linked nucleosides, and wherein oligonucleotide shares at least 85% identity with any one of SEQ ID NOs: 1-466, SEQ ID NOs: 893-1338, SEQ ID NOs: 1342-1366, SEQ ID NOs: 1392-1664, or SEQ ID NOs: 10655-10669, SEQ ID NOs: 1676-1851, SEQ ID NOs: 2028-2529, SEQ ID NOs: 3046-3221, SEQ ID NOs: 3398-3899, and SEQ ID NOs: 4402-4530, or SEQ ID NOs: 4531-5794, SEQ ID NOs: 7059-8322, or SEQ ID NOs; 9596-9603, or a pharmaceutically acceptable salt thereof;
wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or
wherein at least one (i.e., one or more) nucleoside is substituted with a component selected from the group consisting of a 2′-O-(2-methoxyethyl) nucleoside, a 2′-O-methyl nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA)
optionally, wherein the oligonucleotide further comprises a spacer.

In various embodiments, nucleoside linkages that link a base at position 3 or position 4 of the oligonucleotide are phosphodiester linkages. In various embodiments, only one nucleoside linkage that links a base at position 3 or position 4 of the oligonucleotide is a phosphodiester linkage. In various embodiments, nucleoside linkages that link bases at both position 3 and position 4 of the oligonucleotide are phosphodiester linkages. In various embodiments, one or more bases immediately preceding a spacer in the oligonucleotide are linked through phosphodiester bonds. In various embodiments, only the base immediately preceding the spacer in the oligonucleotide is linked to the spacer through a phosphodiester bond. In various embodiments, the base immediately preceding the spacer in the oligonucleotide is further linked to a further preceding base through a phosphodiester bond. In various embodiments, the oligonucleotide comprises a second spacer, wherein a base immediately preceding the second spacer is linked to a further preceding base through a phosphodiester bond. In various embodiments, one or more bases immediately succeeding a spacer in the oligonucleotide are linked through phosphodiester bonds. In various embodiments, only the base immediately succeeding the spacer in the oligonucleotide is linked to the spacer through a phosphodiester bond. In various embodiments, two bases immediately preceding the spacer in the oligonucleotide are linked through phosphodiester bonds. In various embodiments, one or more bases immediately preceding a spacer in the oligonucleotide are linked through phosphodiester bonds and wherein one or more bases immediately succeeding the spacer in the oligonucleotide are linked through phosphodiester bonds. In various embodiments, one base immediately preceding the spacer and one base immediately succeeding the spacer are linked through phosphodiester bonds. In various embodiments, the oligonucleotide includes a second spacer, and wherein one or more bases immediately preceding the second spacer in the oligonucleotide are linked through phosphodiester bonds and wherein one or more bases immediately succeeding the second spacer in the oligonucleotide are linked through phosphodiester bonds. In various embodiments, one base immediately preceding the second spacer and one base immediately succeeding the second spacer are linked through phosphodiester bonds.

In various embodiments, the oligonucleotide comprises a range of bases that are linked through phosphodiester bonds, the range of bases comprising at least two bases. In various embodiments, the oligonucleotide comprises a range of bases that are linked through phosphodiester bonds, the range of bases comprising at least five bases. In various embodiments, the oligonucleotide comprises two or more spacers, and wherein the range of bases are positioned between the at least two spacers.

In various embodiments, the oligonucleotide is any one of a 19mer, 21mer, 23mer, or 25mer.

In various embodiments, at least one (i.e., one or more) internucleoside linkage of the oligonucleotide is a phosphorothioate linkage. In various embodiments, all internucleoside linkages of the oligonucleotide are phosphorothioate linkages.

Additionally disclosed herein is an oligonucleotide and a pharmaceutically acceptable excipient, the oligonucleotide comprising a sequence that is at least 85% complementary to an equal length portion of any one of SEQ ID NO: 1339 or SEQ ID NO: 1341, SEQ ID NO: 3032-3043, SEQ ID NO: 9587-9595, or SEQ ID NO: 9698-9707, a sequence having 90% identity thereof, or to a 15 to 50 contiguous nucleobase portion thereof, optionally wherein the oligonucleotide comprises a spacer and wherein the oligonucleotide is capable of increasing, restoring, or stabilizing expression of one or more of STMN2, KCNQ2, or UNC13A mRNA capable of translation of a functional protein in a cell or a human patient of an immune-mediated demyelinating disease, and wherein the level of increase, restoration, or stabilization of expression and/or activity and/or function is sufficient for use of the oligonucleotide as a medicament for the treatment of the immune-mediated demyelinating disease.

In various embodiments, the pharmaceutical composition disclosed herein, or the oligonucleotide disclosed herein, wherein the oligonucleotide comprises one or more chiral centers and/or double bonds.

In various embodiments, the pharmaceutical composition disclosed herein, or the oligonucleotide disclosed herein, wherein the oligonucleotide exist as stereoisomers selected from geometric isomers, enantiomers, and diastereomers.

Additionally disclosed herein is a method of treating a neurological disease and/or a neuropathy in a patient in need thereof, the method comprising administering to a patient in need thereof a therapeutically effective amount of a pharmaceutical composition, in combination with a second therapeutic agent.

In various embodiments, the second therapeutic agent is selected from Riluzole (Rilutek), PrimeC, Edaravone (Radicava), rivastigmine, donepezil, galantamine, selective serotonin reuptake inhibitor, antipsychotic agents, cholinesterase inhibitors, memantine, benzodiazepine antianxiety drugs, AMX0035 (ELYBRIO), ZILUCOPLAN (RA101495), pridopidine, dual AON intrathecal administration (e.g., BIIB067, BIIB078, and BIIB105), BIIB100, levodopa/carbidopa, dopaminergic agents (e.g., ropinirole, pramipexole, rotigotine), medroxyprogesetrone, KCNQ2/KCNQ3 openers (e.g., retigabine, XEN1101, or QRL-101), bioactive scaffolds, anticonvulsants and psychostimulant agents, a therapy (e.g., selected from breathing care, physical therapy, occupational therapy, speech therapy, nutritional support), deep brain stimulation, levodopa and carbidopa (duopa, rytary, Sinemet, inbrija), istradefylline (nourianz), safinamide (xadago), pramipexole (Mirapex), rotigotine (neupro), ropinirole (requip), amantadine (gocovri, Symmetrel, osmolex), benztropine (Cogentin), trihexyphenidyl (artane), selegiline (eldepryl, zelapar), rasagiline, entacapone (comtan), opicapone (ongentys), tolcapone (tasmar), apomorphine (apokyn, kynmobi), exenatide, lingzhi, BIIB054, BIIB094, Caffeine, sarizotan, embryonic dopamine cell implantation, aducanamab (Aduhlem), memantine (Namenda), Donepezil (Aricept), Rivastigmine (Exelon), Galantamine (razadyne), Namzeric, Suvorexant (belsomra), lecanemab, olanzapine (Zyprexa), quetiapine (Seroquel), SSRIs (citalopram (Cipramil), dapoxetine (Priligy), escitalopram (Cipralex), fluoxetine (Prozac or Oxactin), fluvoxamine (Faverin), paroxetine (Seroxat), sertraline (Lustral), vortioxetine (Brintellix)), divalproex sodium (Depakote), carbamazepine (Tegretol), medroxyprogestrone, Brivaracetam (briviact), cannabidiol (epidiolex), carbamazepine (carbatrol, Tegretol), cenobamate (xcopri), diazepam (valium), lorazepam (Ativan), clonazepam (klonopin), eslicarbazepine (aptiom), ethosuximide (zarontin), felbamate (felbatol), fenfluramine (fintepla), lacosamide (VIMPAT), lamotrigine (Lamictal), levetiracetam (Keppra), oxcarbazepine (oxtellar xr, Trileptal), perampanel (fycompa), phenobarbital, phenytoin (dilantin), pregabalin (lyrica), tiagabine (gabitril), topiramate (topamax), valproate (depakene, depakote), and/or zonisamide (zonegran), for treating said neurologic disease.

Additionally disclosed herein is a method of treating a neurological disease and/or a neuropathy in a patient in need thereof, the method comprising administering to a patient in need thereof a therapeutically effective amount of a pharmaceutical composition, wherein at least one nucleoside linkage of the oligonucleotide is a non-natural linkage, optionally wherein the oligonucleotide comprises a spacer, and wherein the oligonucleotide further comprises a targeting or conjugate moiety selected from cholesterol, lipoic acid, panthothenic acid, polyethylene glycol, and an antibody for crossing the blood brain barrier.

In various embodiments, the spacer is a nucleoside-replacement group comprising a non-sugar substitute that is incapable of linking to a nucleotide base. In various embodiments, the spacer is located between positions 10 and 15 of the oligonucleotide. In various embodiments, the spacer is located between positions 7 and 11 of the oligonucleotide. In various embodiments, the oligonucleotide further comprises a second spacer, wherein the second spacer is located between positions 14 and 22 of the oligonucleotide. In various embodiments, the spacer and the second spacer are separated by at least 5 nucleobases, at least 6 nucleobases, or at least 7 nucleobases in the oligonucleotide. In various embodiments, the spacer is located between positions 7 and 9 of the oligonucleotide, and wherein the second spacer is located between positions 15 and 18 of the oligonucleotide. In various embodiments, the spacer is located at position 8 of the oligonucleotide, and wherein the second spacer is located at position 16 of the oligonucleotide. In various embodiments, the oligonucleotide further comprises a third spacer, wherein the third spacer is located between positions 21 and 24 of the oligonucleotide. In various embodiments, the spacer is located between positions 2 and 5 of the oligonucleotide. In various embodiments, the oligonucleotide further comprises a second spacer, wherein the second spacer is located between positions 8 and 12 of the oligonucleotide. In various embodiments, the oligonucleotide further comprises a third spacer, wherein the third spacer is located between positions 18 and 22 of the oligonucleotide. In various embodiments, the oligonucleotide further comprises a second spacer and a third spacer, wherein the three spacers are located at positions in the oligonucleotide such that each segment of the oligonucleotide has at most 7 linked nucleosides. In various embodiments, at least two of the three spacers are adjacent to a guanine nucleobase. In various embodiments, each of the at least two of the three spacers immediately precede a guanine nucleobase. In various embodiments, each of the first, second or third spacers is a nucleoside-replacement group comprising a non-sugar substitute wherein the non-sugar substitute does not contain a ketone, aldehyde, ketal, hemiketal, acetal, hemiacetal, aminal or hemiaminal moiety and is incapable of forming a covalent bond with a nucleotide base.

In various embodiments, each of the first, second or third spacers is independently represented by Formula (X), wherein:

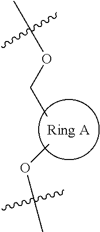

Ring A is an optionally substituted 4-8 member monocyclic cycloalkyl group or a 4-8 member monocyclic heterocyclyl group, wherein the heterocyclyl group contains 1 or 2 heteroatoms selected from O, S and N, provided that A is not capable of forming a covalent bond to a nucleobase; and
the

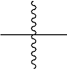

symbol represents the point of connection to an internucleoside linkage.

In various embodiments, each of the first, second or third spacers is independently represented by Formula (Xa), wherein:

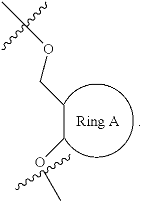

In various embodiments, ring A is an optionally substituted 4-8 member monocyclic cycloalkyl group selected from cyclobutyl, cyclopentyl, cyclohexyl, cycloheptyl and cyclooctyl; or a 4-8 member monocyclic heterocyclyl group, selected from oxetanyl, tetrahydrofuranyl, tetrahydropyranyl, 1,4-dioxanyl, pyrolidinyl, piperidinyl, piperazinyl, morpholinyl and azepanyl.

In various embodiments, ring A is tetrahydrofuranyl. In various embodiments, ring A is tetrahydropyranyl.

In various embodiments, each of the first, second or third spacers is independently represented by Formula (I), wherein:

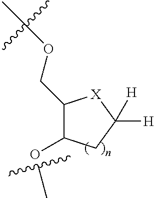

X is selected from —CH₂— and —O—; and
n is 0, 1, 2 or 3.

In various embodiments, each of the first, second or third spacers is independently represented by Formula (I′), wherein:

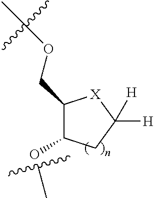

In various embodiments, each of the first, second or third spacers is independently represented by Formula (Ia), wherein:

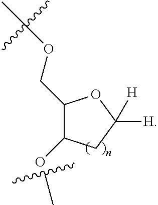

In various embodiments, each of the first, second or third spacers is independently represented by Formula (Ia′), wherein:

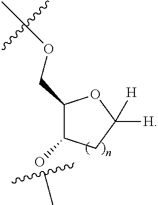

In various embodiments, each of the first, second or third spacers is independently represented by Formula II, wherein:

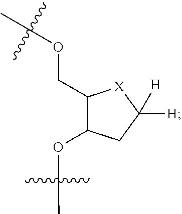

and
X is selected from —CH₂— and —O—.

In various embodiments, each of the first, second or third spacers is independently represented by Formula II′, wherein:

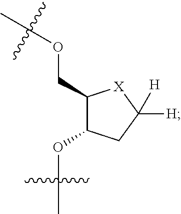

and
X is selected from —CH₂— and —O—.

In various embodiments, each of the first, second or third spacers is independently represented by Formula (IIa), wherein:

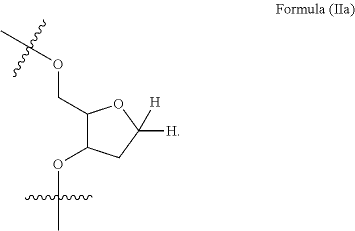

In various embodiments, each of the first, second or third spacers is independently represented by Formula (Iia′), wherein:

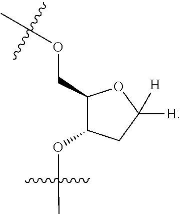

In some embodiments, the spacer is represented by Formula (IIi), wherein:

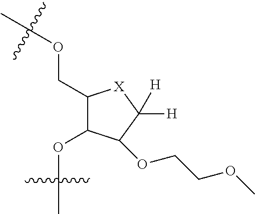

X is selected from —CH₂— and —O—.

In some embodiments, the spacer is represented by Formula (IIi′), wherein:

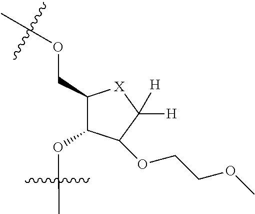

X is selected from —CH₂— and —O.

In some embodiments, the spacer is represented by Formula (IIib), wherein:

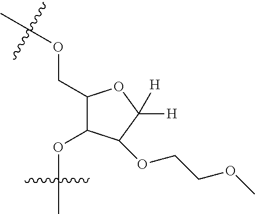 [SMILES: *OCC1OCC(OCCOC)C1O*]

In some embodiments, the spacer is represented by Formula (IIib′), wherein:

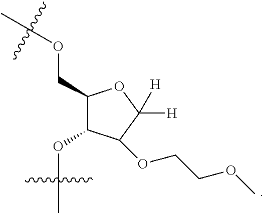 [SMILES: *OC[C@H]1OCC(OCCOC)[C@@H]1O*]

In various embodiments, each of the first, second or third spacers is independently represented by Formula III, wherein:

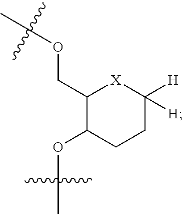

and
X is selected from —CH₂— and —O—.

T In various embodiments, each of the first, second or third spacers is independently represented by Formula III′, wherein:

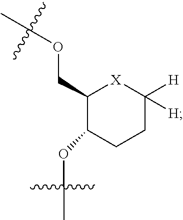

and
X is selected from —CH₂— and —O—.

In various embodiments, each of the first, second or third spacers is independently represented by Formula (IIIa), wherein:

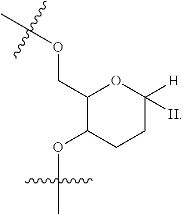

In various embodiments, each of the first, second or third spacers is independently represented by Formula (IIIa′), wherein:

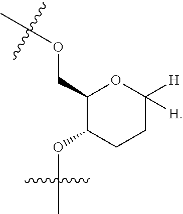

In various embodiments, the oligonucleotide comprising the spacer has a GC content of at least 10%. In various embodiments, the oligonucleotide comprising the spacer has a GC content of at least 20%. In various embodiments, the oligonucleotide comprising the spacer has a GC content of at least 25%. In various embodiments, the oligonucleotide comprising the spacer has a GC content of at least 30%. In various embodiments, the oligonucleotide comprising the spacer has a GC content of at least 40%. In various embodiments, the oligonucleotide comprising the spacer has a GC content of at least 50%.

### BRIEF DESCRIPTION OF THE DRAWINGS

FIG. 1 is a schematic depiction of portions of the STMN2 transcript and STMN2 antisense oligonucleotides that are designed to target certain portions of the STMN2 transcript.

FIG. 2 is a bar graph showing the results of RT-qPCR analysis of TDP43 and STMN2 full-length mRNA levels in the presence of TDP43 antisense, and restoration of the full-length STMN2 transcript in the presence of 6 different STMN2 parent oligonucleotides (SEQ ID NO: 36, SEQ ID NO: 55, SEQ ID NO: 177, SEQ ID NO: 203, SEQ ID NO: 244, and SEQ ID NO: 395).

FIG. 3 is a bar graph showing the results of RT-qPCR analysis of STMN2 transcript with cryptic exon mRNA levels in the presence of TDP43 antisense, and reduction of the STMN2 transcript with cryptic exon mRNA levels in the presence of 6 different STMN2 parent oligonucleotides (SEQ ID NO: 173, SEQ ID NO: 181, SEQ ID NO: 197, SEQ ID NO: 215, SEQ ID NO: 385, and SEQ ID NO: 400).

FIG. 4 is a bar graph showing the results of RT-qPCR analysis of STMN2 full-length mRNA levels in the presence of TDP43 antisense, and restoration of the full-length STMN2 transcript in the presence of 6 different STMN2 parent oligonucleotides (SEQ ID NO: 173, SEQ ID NO: 181, SEQ ID NO: 197, SEQ ID NO: 215, SEQ ID NO: 385, and SEQ ID NO: 400).

FIG. 5A is a bar graph showing the results of RT-qPCR analysis of STMN2 transcript with cryptic exon mRNA levels in the presence of TDP43 antisense, and reduction of the STMN2 transcript with cryptic exon mRNA levels in the presence of 6 different STMN2 parent oligonucleotides (SEQ ID NO: 185, SEQ ID NO: 209, SEQ ID NO: 237, SEQ ID NO: 252, SEQ ID NO: 380, and SEQ ID NO: 390).

FIG. 5B is a bar graph showing the results of RT-qPCR analysis of STMN2 full-length mRNA levels in the presence of TDP43 antisense, and restoration of the full-length STMN2 transcript in the presence of 6 different STMN2 parent oligonucleotides (SEQ ID NO: 185, SEQ ID NO: 209, SEQ ID NO: 237, SEQ ID NO: 252, SEQ ID NO: 380, and SEQ ID NO: 390).

FIG. 6A is a bar graph showing the results of RT-qPCR analysis of STMN2 transcript with cryptic exon mRNA levels in the presence of TDP43 antisense, and reduction of the STMN2 transcript with cryptic exon mRNA levels in the presence of 2 different STMN2 parent oligonucleotides (SEQ ID NO: 144 and SEQ ID NO: 237) over two duplicate experiments.

FIG. 6B is a bar graph showing the results of RT-qPCR analysis of STMN2 full-length mRNA levels in the presence of TDP43 antisense, and restoration of the full-length STMN2 transcript in the presence of 2 different STMN2 parent oligonucleotides (SEQ ID NO: 144 and SEQ ID NO: 237) over two duplicate experiments.

FIG. 7A is a bar graph showing the results of RT-qPCR analysis of STMN2 transcript with cryptic exon mRNA levels in the presence of TDP43 antisense, and reduction of the STMN2 transcript with cryptic exon mRNA levels in the presence of 5 different STMN2 parent oligonucleotides (SEQ ID NO: 36, SEQ ID NO: 173, SEQ ID NO: 177, SEQ ID NO: 181, and SEQ ID NO: 185).

FIG. 7B is a bar graph showing the results of RT-qPCR analysis of STMN2 full-length mRNA levels in the presence of TDP43 antisense, and restoration of the full-length STMN2 transcript in the presence of 5 different STMN2 parent oligonucleotides (SEQ ID NO: 36, SEQ ID NO: 173, SEQ ID NO: 177, SEQ ID NO: 181, and SEQ ID NO: 185).

FIG. 8A is a bar graph showing the results of RT-qPCR analysis of STMN2 transcript with cryptic exon mRNA levels in the presence of TDP43 antisense, and reduction of the STMN2 transcript with cryptic exon mRNA levels in the presence of 5 different STMN2 parent oligonucleotides (SEQ ID NO: 197, SEQ ID NO: 203, SEQ ID NO: 237, SEQ ID NO: 380, and SEQ ID NO: 395).

FIG. 8B is a bar graph showing the results of RT-qPCR analysis of STMN2 full-length mRNA levels in the presence of TDP43 antisense, and restoration of the full-length STMN2 transcript in the presence of 5 different STMN2 parent oligonucleotides (SEQ ID NO: 197, SEQ ID NO: 203, SEQ ID NO: 237, SEQ ID NO: 380, and SEQ ID NO: 395).

FIG. 9A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels in the presence of 3 different STMN2 parent oligonucleotides (SEQ ID NO: 144, SEQ ID NO: 173, and SEQ ID NO: 237).

FIG. 9B is a bar graph showing the results of RT-qPCR analysis of TDP43 and STMN2 full-length mRNA levels in the presence of TDP43 siRNA and TDP43 antisense, and restoration of the full-length STMN2 transcript in the presence of 3 different STMN2 parent oligonucleotides (SEQ ID NO: 144, SEQ ID NO: 173, and SEQ ID NO: 237).

FIG. 10A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels across different dosages of a SEQ ID NO: 181 STMN2 parent oligonucleotide.

FIG. 10B is a bar graph showing the results of RT-qPCR analysis of TDP43 and STMN2 full-length mRNA levels in the presence of TDP43 siRNA and TDP43 antisense, and restoration of the full-length STMN2 transcript across different dosages of a SEQ ID NO: 181 STMN2 parent oligonucleotide.

FIG. 11A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels across different dosages of a SEQ ID NO: 185 STMN2 parent oligonucleotide.

FIG. 11B is a bar graph showing the results of RT-qPCR analysis of TDP43 and STMN2 full-length mRNA levels in the presence of TDP43 siRNA and TDP43 antisense, and restoration of the full-length STMN2 transcript across different dosages of a SEQ ID NO: 185 STMN2 parent oligonucleotide.

FIG. 12A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels across different dosages of a SEQ ID NO: 197 STMN2 parent oligonucleotide.

FIG. 12B is a bar graph showing the results of RT-qPCR analysis of TDP43 and STMN2 full-length mRNA levels in the presence of TDP43 siRNA and TDP43 antisense, and restoration of the full-length STMN2 transcript across different dosages of a SEQ ID NO: 197 STMN2 parent oligonucleotide.

FIG. 13A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels across different dosages of a SEQ ID NO: 144 STMN2 parent oligonucleotide.

FIG. 13B is a bar graph showing the results of RT-qPCR analysis of TDP43 and STMN2 full-length mRNA levels in the presence of TDP43 siRNA and TDP43 antisense, and restoration of the full-length STMN2 transcript across different dosages of a SEQ ID NO: 144 STMN2 parent oligonucleotide.

FIG. 14A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels across different dosages of a SEQ ID NO: 173 STMN2 parent oligonucleotide.

FIG. 14B is a bar graph showing the results of RT-qPCR analysis of TDP43 and STMN2 full-length mRNA levels in the presence of TDP43 siRNA and TDP43 antisense, and restoration of the full-length STMN2 transcript across different dosages of a SEQ ID NO: 173 STMN2 parent oligonucleotide.

FIG. 15A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels across different dosages of a SEQ ID NO: 237 STMN2 parent oligonucleotide.

FIG. 15B is a bar graph showing the results of RT-qPCR analysis of TDP43 and STMN2 full-length mRNA levels in the presence of TDP43 siRNA and TDP43 antisense, and restoration of the full-length STMN2 transcript across different dosages of a SEQ ID NO: 237 STMN2 parent oligonucleotide.

FIG. 16 is a protein blot and quantified bar graph showing the normalized quantity of STMN2 full-length mRNA levels in the presence of TDP43 siRNA and TDP43 antisense, and restoration of the full-length STMN2 transcript for 2 different STMN2 parent oligonucleotides (SEQ ID NO: 173 and SEQ ID NO: 237).

FIG. 17A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels using different variants of a SEQ ID NO: 237 STMN2 parent oligonucleotide.

FIG. 17B is a bar graph showing the results of RT-qPCR analysis of TDP43 and STMN2 full-length mRNA levels in the presence of TDP43 siRNA and TDP43 antisense, and restoration of the full-length STMN2 transcript using different variants of a SEQ ID NO: 237 STMN2 parent oligonucleotide.

FIG. 18A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels using different variants of a SEQ ID NO: 185 STMN2 parent oligonucleotide.

FIG. 18B is a bar graph showing the results of RT-qPCR analysis of TDP43 and STMN2 full-length mRNA levels in the presence of TDP43 siRNA and TDP43 antisense, and restoration of the full-length STMN2 transcript using different variants of a SEQ ID NO: 185 STMN2 parent oligonucleotide.

FIG. 19A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels using different variants of a SEQ ID NO: 173 STMN2 parent oligonucleotide.

FIG. 19B is a bar graph showing the results of RT-qPCR analysis of TDP43 and STMN2 full-length mRNA levels in the presence of TDP43 siRNA and TDP43 antisense, and restoration of the full-length STMN2 transcript using different variants of a SEQ ID NO: 173 STMN2 parent oligonucleotide.

FIG. 20A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels using different variants of a SEQ ID NO: 237 STMN2 parent oligonucleotide.

FIG. 20B is a bar graph showing the results of RT-qPCR analysis of TDP43 and STMN2 full-length mRNA levels in the presence of TDP43 siRNA and TDP43 antisense, and restoration of the full-length STMN2 transcript using different variants of a SEQ ID NO: 237 STMN2 parent oligonucleotide.

FIG. 21A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels using different variants of a SEQ ID NO: 173 STMN2 parent oligonucleotide.

FIG. 21B is a bar graph showing the results of RT-qPCR analysis of TDP43 and STMN2 full-length mRNA levels in the presence of TDP43 siRNA and TDP43 antisense, and restoration of the full-length STMN2 transcript using different variants of a SEQ ID NO: 173 STMN2 parent oligonucleotide.

FIG. 22A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels using different variants of a SEQ ID NO: 144 STMN2 parent oligonucleotide.

FIG. 22B is a bar graph showing the results of RT-qPCR analysis of TDP43 and STMN2 full-length mRNA levels in the presence of TDP43 siRNA and TDP43 antisense, and restoration of the full-length STMN2 transcript using different variants of a SEQ ID NO: 144 STMN2 parent oligonucleotide.

FIG. 23 is a bar graph showing reversal of cryptic exon induction using SEQ ID NO: 237 STMN2 parent oligonucleotide even in view of increasing proteasome inhibition.

FIG. 24 shows the dose response curve illustrating increasing restoration of full length STMN2 transcript with increasing concentrations of STMN2 AON.

FIG. 25A shows a protein blot assay demonstrating the qualitative increase of full length STMN2 protein in response to higher concentrations of STMN2 AON.

FIG. 25B shows the quantitated levels of full length STMN2 protein normalized to GAPDH in response to different concentrations of STMN2 AON.

FIG. 26A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels across different dosages of STMN2 AONs including a SEQ ID NO: 144 AON, a SEQ ID NO: 144 AON with two spacers (SEQ ID NO: 1589), a SEQ ID NO: 173 AON, a SEQ ID NO: 173 with two spacers (SEQ ID NO: 1590), a SEQ ID NO: 237 AON, and a SEQ ID NO: 237 AON with two spacers (SEQ ID NO: 1591).

FIG. 26B is a bar graph showing the results of RT-qPCR analysis of TDP43 and STMN2 full-length mRNA levels in the presence of TDP43 siRNA and TDP43 antisense, and restoration of the full-length STMN2 transcript across different dosages of STMN2 AONs including a SEQ ID NO: 144 AON, a SEQ ID NO: 144 AON with two spacers (SEQ ID NO: 1589), a SEQ ID NO: 173 AON, a SEQ ID NO: 173 with two spacers (SEQ ID NO: 1590), a SEQ ID NO: 237 AON, and a SEQ ID NO: 237 AON with two spacers (SEQ ID NO: 1591).

FIG. 27A is a bar graph showing the results of RT-qPCR analysis of STMN2 transcript with cryptic exon mRNA levels in the presence of TDP43 antisense, and reduction of the STMN2 transcript with cryptic exon mRNA levels across different dosages of STMN2 AONs including SEQ ID NO: 173, SEQ ID NO: 1608, SEQ ID NO: 1609, SEQ ID NO: 1610, SEQ ID NO: 1611, SEQ ID NO: 1612, SEQ ID NO: 1613, SEQ ID NO: 1614, SEQ ID NO: 1615, SEQ ID NO: 15%, SEQ ID NO: 1597, and SEQ ID NO: 1418.

FIG. 27B is a bar graph showing the results of RT-qPCR analysis of TDP43 and STMN2 full-length mRNA levels in the presence of TDP43 antisense, and restoration of the full-length STMN2 transcript across different dosages of STMN2 AONs including SEQ ID NO: 173, SEQ ID NO: 1608, SEQ ID NO: 1609, SEQ ID NO: 1610, SEQ ID NO: 1611, SEQ ID NO: 1612, SEQ ID NO: 1613, SEQ ID NO: 1614, SEQ ID NO: 1615, SEQ ID NO: 15%, SEQ ID NO: 1597, and SEQ ID NO: 1418.

FIG. 28A is a bar graph showing the results of RT-qPCR analysis of STMN2 transcript with cryptic exon mRNA levels in the presence of TDP43 antisense, and reduction of the STMN2 transcript with cryptic exon mRNA levels across different dosages of STMN2 AONs including SEQ ID NO: 173, SEQ ID NO: 1632, SEQ ID NO: 1346, SEQ ID NO: 1631, SEQ ID NO: 1353, and SEQ ID NO: 1598.

FIG. 28B is a bar graph showing the results of RT-qPCR analysis of TDP43 and STMN2 full-length mRNA levels in the presence of TDP43 antisense, and restoration of the full-length STMN2 transcript across different dosages of STMN2 AONs including SEQ ID NO: 173, SEQ ID NO: 1632, SEQ ID NO: 1346, SEQ ID NO: 1631, SEQ ID NO: 1353, and SEQ ID NO: 1598.

FIG. 29A is a bar graph showing the results of RT-qPCR analysis of STMN2 transcript with cryptic exon mRNA levels in the presence of TDP43 antisense, and reduction of the STMN2 transcript with cryptic exon mRNA levels across different dosages of STMN2 AONs including SEQ ID NO: 173 and SEQ ID NO: 1610.

FIG. 29B is a bar graph showing the results of RT-qPCR analysis of TDP43 and STMN2 full-length mRNA levels in the presence of TDP43 antisense, and restoration of the full-length STMN2 transcript across different dosages of STMN2 AONs including SEQ ID NO: 173 and SEQ ID NO: 1610.

FIG. 30A is a bar graph showing the results of RT-qPCR analysis of STMN2 transcript with cryptic exon mRNA levels in the presence of TDP43 antisense, and reduction of the STMN2 transcript with cryptic exon mRNA levels across different dosages of STMN2 AONs including SEQ ID NO: 185 and SEQ ID NO: 1635.

FIG. 30B is a bar graph showing the results of RT-qPCR analysis of TDP43 and STMN2 full-length mRNA levels in the presence of TDP43 antisense, and restoration of the full-length STMN2 transcript across different dosages of STMN2 AONs including SEQ ID NO: 185 and SEQ ID NO: 1635.

FIG. 31A is a bar graph showing the results of RT-qPCR analysis of STMN2 transcript with cryptic exon mRNA levels in the presence of TDP43 antisense, and reduction of the STMN2 transcript with cryptic exon mRNA levels across different dosages of STMN2 AONs including SEQ ID NO: 1347, SEQ ID NO: 1633, and SEQ ID NO: 1634.

FIG. 31B is a bar graph showing the results of RT-qPCR analysis of TDP43 and STMN2 full-length mRNA levels in the presence of TDP43 antisense, and restoration of the fill-length STMN2 transcript across different dosages of STMN2 AONs including SEQ ID NO: 1347, SEQ ID NO: 1633, and SEQ ID NO: 1634.

FIG. 32A is a bar graph showing the results of RT-qPCR analysis of STMN2 transcript with cryptic exon mRNA levels in the presence of TDP43 antisense, and reduction of the STMN2 transcript with cryptic exon mRNA levels across different dosages of STMN2 AONs including SEQ ID NO: 197, SEQ ID NO: 1617, SEQ ID NO: 1618, and SEQ ID NO: 1619.

FIG. 32B is a bar graph showing the results of RT-qPCR analysis of TDP43 and STMN2 full-length mRNA levels in the presence of TDP43 antisense, and restoration of the fill-length STMN2 transcript across different dosages of STMN2 AONs including SEQ ID NO: 197, SEQ ID NO: 1617, SEQ ID NO: 1618, and SEQ ID NO: 1619.

FIG. 33A is a bar graph showing the results of RT-qPCR analysis of STMN2 transcript with cryptic exon mRNA levels in the presence of TDP43 antisense, and reduction of the STMN2 transcript with cryptic exon mRNA levels across different dosages of STMN2 AONs including SEQ ID NO: 252, SEQ ID NO: 1650, SEQ ID NO: 1434, SEQ ID NO: 1651, and SEQ ID NO: 1620.

FIG. 33B is a bar graph showing the results of RT-qPCR analysis of TDP43 and STMN2 full-length mRNA levels in the presence of TDP43 antisense, and restoration of the fill-length STMN2 transcript across different dosages of STMN2 AONs including SEQ ID NO: 252, SEQ ID NO: 1650, SEQ ID NO: 1434, SEQ ID NO: 1651, and SEQ ID NO: 1620.

FIG. 34A is a bar graph showing the results of RT-qPCR analysis of STMN2 transcript with cryptic exon mRNA levels in the presence of TDP43 antisense, and reduction of the STMN2 transcript with cryptic exon mRNA levels across different dosages of STMN2 AONs including SEQ ID NO: 1434 and SEQ ID NO: 1620.

FIG. 34B is a bar graph showing the results of RT-qPCR analysis of TDP43 and STMN2 full-length mRNA levels in the presence of TDP43 antisense, and restoration of the fill-length STMN2 transcript across different dosages of STMN2 AONs including SEQ ID NO: 1434 and SEQ ID NO: 1620.

FIG. 35 is a bar graph showing normalized STMN2 protein levels following treatment with TDP43 antisense and restoration using STMN2 AONs including SEQ ID NO: 144, SEQ ID NO: 1589, SEQ ID NO: 173, SEQ ID NO: 1616, SEQ ID NO: 237, and SEQ ID NO: 1591.

### DETAILED DESCRIPTION

The features and other details of the disclosure will now be more particularly described. Certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

Disclosed herein are oligonucleotides capable of targeting a region of a transcript transcribed from a gene. In various embodiments, such oligonucleotides target a STMN2 transcript. In various embodiments, such oligonucleotides target a KCNQ2 transcript. In various embodiments, such oligonucleotides target a UNC13A transcript. In various embodiments, such oligonucleotides target a SMN2 transcript. Additionally disclosed herein are oligonucleotides, including antisense oligonucleotide sequences, and methods for treating neurological diseases, such as amyotrophic lateral sclerosis and frontotemporal dementia, and/or neuropathies such as chemotherapy induced neuropathy, using same. In one embodiment, the oligonucleotides target a cryptic exon sequence of STMN2 transcripts, thereby reducing levels of STMN2 transcripts with the cryptic exon sequence. In various embodiments, the oligonucleotides target a sequence of KCNQ2 transcripts resulting in the reduction of levels of mis-spliced KCNQ2 transcripts. In various embodiments, the oligonucleotides target a sequence of UNC13A transcripts resulting in the reduction of levels of mis-spliced UNC13A transcripts. In various embodiments, the oligonucleotides target a sequence of SMN2 transcripts resulting in the reduction of levels of mis-spliced SMN2 transcripts. Also disclosed are pharmaceutical compositions comprising STMN2 oligonucleotides that target a region of STMN2 transcripts that comprise a cryptic exon, for treating neurological diseases and/or neuropathies; and manufacture of medicaments containing a disclosed STMN2 oligonucleotide that targets a region of STMN2 transcripts that comprise a cryptic exon to be used in treating a neurological disease and/or neuropathy. Also disclosed are pharmaceutical compositions comprising KCNQ2 oligonucleotides that target a region of KCNQ2 transcripts, for treating neurological diseases and/or neuropathies; and manufacture of medicaments containing a disclosed KCNQ2 oligonucleotide that targets a region of KCNQ2 transcripts to be used in treating a neurological disease and/or neuropathy. Also disclosed are pharmaceutical compositions comprising UNC13A oligonucleotides that target a region of UNC13A transcripts, for treating neurological diseases and/or neuropathies; and manufacture of medicaments containing a disclosed UNC13A oligonucleotide that targets a region of UNC13A transcripts to be used in treating a neurological disease and/or neuropathy. Also disclosed are pharmaceutical compositions comprising SMN2 oligonucleotides that target a region of SMN2 transcripts, for treating neurological diseases and/or neuropathies; and manufacture of medicaments containing a disclosed SMN2 oligonucleotide that targets a region of SMN2 transcripts to be used in treating a neurological disease and/or neuropathy.

### Definitions

The terms “treat,” “treatment,” “treating,” and the like are used herein to generally mean obtaining a desired pharmacological and/or physiological effect. The effect may be therapeutic in terms of partially or completely curing a disease and/or adverse effect attributed to the disease. The term “treatment” as used herein covers any treatment of a disease in a mammal, particularly a human, and includes: (a) inhibiting the disease, i.e., preventing the disease from increasing in severity or scope; (b) relieving the disease, i.e., causing partial or complete amelioration of the disease; or (c) preventing relapse of the disease, i.e., preventing the disease from returning to an active state following previous successful treatment of symptoms of the disease or treatment of the disease.

“Preventing” includes delaying the onset of clinical symptoms, complications, or biochemical indicia of the state, disorder, disease, or condition developing in a subject that may be afflicted with or predisposed to the state, disorder, disease, or condition but does not yet experience or display clinical or subclinical symptoms of the state, disorder, disease, or condition. “Preventing” includes prophylactically treating a state, disorder, disease, or condition in or developing in a subject, including prophylactically treating clinical symptoms, complications, or biochemical indicia of the state, disorder, disease, or condition in or developing in a subject.

The term “pharmaceutically acceptable carrier” or “pharmaceutically acceptable excipient” as used herein interchangeably refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one biologically active compound, for example, a STMN2 antisense oligonucleotide (AON), as disclosed herein formulated together with one or more pharmaceutically acceptable excipients.

“Individual,” “patient,” or “subject” are used interchangeably and include any animal, including mammals, preferably mice, rats, other rodents, rabbits, dogs, cats, swine, cattle, sheep, horses, or non-human primates, and most preferably humans. The compounds of the invention can be administered to a mammal, such as a human, but can also be other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, non-human primates, and the like). In some embodiments, the mammal treated in the methods of the invention is desirably a mammal in whom modulation of STMN2 expression and/or activity is desired. In some embodiments, the mammal treated in the methods of the invention is desirably a mammal in whom modulation of KCNQ2 expression and/or activity is desired. In some embodiments, the mammal treated in the methods of the invention is desirably a mammal in whom modulation of UNC13A expression and/or activity is desired. In some embodiments, the mammal treated in the methods of the invention is desirably a mammal in whom modulation of SMN2 expression and/or activity is desired.

As used herein, the phrase “regulated by TDP43” encompasses targets that are directly regulated and indirectly regulated by TDP43. Examples of targets that are directly regulated by TDP43 include STMN2 and UNC13A. An example of a target that is indirectly regulated by TDP43 includes KCNQ2.

As used herein, the term “antisense oligonucleotide” or “AON” encompasses antisense oligonucleotides that target genes or gene products of any of STMN2, KCNQ2, UNC13, and SMN2. “Antisense oligonucleotide” or “AON” encompass any of a parent oligonucleotide, an oligonucleotide variant, an oligonucleotide with one or more spacers, or an oligonucleotide variant with one or more spacers. Examples of antisense oligonucleotides include oligonucleotides comprising a sequence of any one of antisense oligonucleotide comprising any one of SEQ ID NOs: 1-466, SEQ ID NO: 893-1338, SEQ ID NOs: 1342-1366, SEQ ID NOs: 1392-1664, SEQ ID NOs: 10655-10669, SEQ ID NOs: 1676-1851, SEQ ID NOs: 2028-2529, SEQ ID NOs: 3046-3221, SEQ ID NO: 3398-3899, SEQ ID NOs: 4402-4530, SEQ ID NOs: 4531-5794, SEQ ID NO: 7059-8322, SEQ ID NOs: 9596-9603, SEQ ID NOs: 9710-10141, SEQ ID NOs: 10574-10651, SEQ ID NOs: 10670-10779, SEQ ID NOs: 10783-10808, and SEQ ID NOs: 10814-10820.

The term “parent oligonucleotide” refers to an oligonucleotide that targets a transcript (e.g., a STMN2, KCNQ2, UNC13A, or SMN2 transcript) and is capable of increasing, restoring, or stabilizing full-length activity e.g., full length expression, for example, full length mRNA and/or full length protein expression. Parent oligonucleotides do not include a spacer. Examples of parent oligonucleotides include oligonucleotides comprising a sequence of any one of SEQ ID NOs: 1-446 and SEQ ID NOs: 893-1338, SEQ ID NOs: 1676-1851, SEQ ID NOs: 2028-2529, and SEQ ID NO: 4531-5794. As described hereafter, oligonucleotide with spacers and oligonucleotide variants are described in relation to a corresponding parent oligonucleotide.

The term “oligonucleotide variant” refers to an oligonucleotide that represents a modified version of a corresponding parent oligonucleotide. For example, an oligonucleotide variant represents a shortened version of a parent oligonucleotide. In various embodiments, an oligonucleotide variant is any one of a 15mer, 16mer, 17mer, 18mer 19mer, 20mer, 21mer, 22mer, 23mer, 24mer, 25mer, 26mer, or 27mer. Examples of oligonucleotide variants include oligonucleotides comprising a sequence of any one of SEQ ID NOs: 1342-1366, SEQ ID NOs: 1392-1521, SEQ ID NOs: 3046-3221, SEQ ID NO: 3398-3899, SEQ ID NO: 7059-8322, and SEQ ID NOs: 9710-10141.

The term “oligonucleotide with one or more spacers” or “oligonucleotide comprising a spacer” refers to an oligonucleotide with at least one spacer. An oligonucleotide with one or more spacers can, in various embodiments, include one spacer, two spacers, three spacers, four spacer, five spacers, six spacers, seven spacers, eight spacers, nine spacers, or ten spacers. In various embodiments, an oligonucleotide comprising one or more spacers includes at least one segment with at most 7 linked nucleosides. For example, as described in a 5′ to 3′ direction, an oligonucleotide comprising a spacer can include a segment with 7 linked nucleosides, followed by a spacer, a second segment with 9 linked nucleosides, followed by a second spacer, and a third segment with 7 linked nucleosides. Here, the first segment of 7 linked nucleosides and the third segment of 7 linked nucleosides each represents segments with at most 7 linked nucleosides. As another example, an oligonucleotide comprising a spacer can include a segment with 10 linked nucleosides, followed by a spacer, a second segment with 10 linked nucleosides, followed by a second spacer, and a third segment with 3 linked nucleosides. Here, the third segment of 3 linked nucleosides represents the segment with at most 7 linked nucleosides. In various embodiments, an oligonucleotide with one or more spacers includes multiple segments with at most 7 linked nucleosides. In various embodiments, every segment of an oligonucleotide with one or more spacers has at most 7 linked nucleosides. For example, the oligonucleotide may be a 23mer and include two spacers that divide the 23mer into three separate segments of 7 linked nucleosides each. Therefore, each segment of the oligonucleotide has at most 7 linked nucleosides.

Generally, oligonucleotides comprising one or more spacers are described in reference to a corresponding parent oligonucleotide or a corresponding oligonucleotide variant. Example oligonucleotides comprising one or spacers include any of SEQ ID NOs: 1417-1420, SEQ ID NOs: 1451-1664, SEQ ID NOs: 4402-4468, SEQ ID NOs: 4469-4476, SEQ ID NOs: 4477-4530, SEQ ID NOs: 9596-9696, SEQ ID NOs: 10574-10640, and SEQ ID NOs: 10644-10651.

### STMN2

As used herein, “STMN2” (also known as Superior Cervical Ganglion-10 Protein, Stathmin-Like 2, SCGN10, SCG10, Neuronal Growth-Associated Protein, Neuron-Specific Growth-Associated Protein, or Protein SCG10 (Superior Cervical Ganglia NEAR Neural Specific 10) refers to the gene or gene products (e.g., protein or mRNA transcript (including pre-mRNA) encoded by the gene) identified by Entrez Gene ID No. 11075 and allelic variants thereof, as well as orthologs found in non-human species (e.g., non-human primates or mice).

The term “STMN2 transcript” refers to a STMN2 transcript comprising a cryptic exon. Such a STMN2 transcript comprising a cryptic exon can be a STMN2 pre-mRNA sequence or a STMN2 mature RNA sequence. The term “STMN2 transcript comprising a cryptic exon” refers to a STMN2 transcript that includes one or more cryptic exon sequences. STMN2 transcript sequences are shown to contain thymine (T), but one of skill in the art will appreciate that thymine (T) can generally be replaced with uracil (U) in RNA sequences.

The term “STMN2 oligonucleotide,” “STMN2 antisense oligonucleotide,” or “STMN2 AON” refers to an oligonucleotide that is capable of increasing, restoring, or stabilizing full-length STMN2 activity e.g., full length STMN2 expression, for example, full length STMN2 mRNA and/or full length STMN2 protein expression. Generally, a STMN2 oligonucleotide reduces the level of mature STMN2 transcripts with a cryptic exon by targeting a STMN2 transcript comprising a cryptic exon. For example, the STMN2 oligonucleotide reduces the level of mature STMN2 transcripts with a cryptic exon by repressing premature polyadenylation of STMN2 pre-mRNA and/or increasing, restoring, or stabilizing activity or function of STMN2. In various embodiments, a STMN2 oligonucleotide comprises a sequence that is at least 85% complementary to an equal length portion of a transcript comprising a sequence at least 90% identity to SEQ ID NO: 1339 or SEQ ID NO: 1341, or a contiguous 15 to 50 nucleobase portion of SEQ ID NO: 1339 or SEQ ID NO: 1341. In various embodiments, a STMN2 oligonucleotide comprises a sequence that is between 85 and 98% complementary to an equal length portion of a transcript comprising a sequence at least 90% identity to SEQ ID NO: 1339 or SEQ ID NO: 1341, or a contiguous 15 to 50 nucleobase portion of SEQ ID NO: 1339 or SEQ ID NO: 1341. STMN2 target sequences are shown to contain thymine (T), but one of skill in the art will appreciate that thymine (T) can generally be replaced with uracil (U) in RNA sequences.

In various embodiments, STMN2 oligonucleotides are characterized by having one or more spacers, where each spacer divides up the STMN2 oligonucleotide into segments of linked nucleosides. In various embodiments, STMN2 oligonucleotides have two spacers. In one embodiment, STMN2 oligonucleotides have two segments of linked nucleosides separated by one spacer. In one embodiment, STMN2 oligonucleotides have three segments of linked nucleosides separated by two spacers. In such embodiments, STMN2 oligonucleotides have one segment with at most 7 linked nucleosides. For example, a STMN2 oligonucleotide may have, from the 5′ to the 3′ end, 5 linked nucleosides, followed by a spacer, 10 linked nucleosides, followed by a second spacer, and 8 linked nucleosides. Thus, the first segment of 5 linked nucleosides satisfies the one segment with at most 7 linked nucleosides. In various embodiments, STMN2 oligonucleotides have three spacers that divide the STMN2 oligonucleotide into four segments. In various embodiments, each of the four segments of the STMN2 oligonucleotide have at most 7 linked nucleosides.

As used herein, the term “STMN2 oligonucleotide” encompasses a “STMN2 parent oligonucleotide,” a “STMN2 oligonucleotide with one or more spacers” (e.g., STMN2 oligonucleotide with two spacers or a STMN2 oligonucleotide with three spacers), a “STMN2 oligonucleotide variant with one or more spacers.” Examples of STMN2 oligonucleotides include oligonucleotides comprising a sequence of any one of SEQ ID NOs: 1-466, SEQ ID NO: 893-1338, SEQ ID NOs: 1342-1366, SEQ ID NOs: 1392-1664, and SEQ ID NOs: 10655-10669.

The term “STMN2 parent oligonucleotide” refers to an oligonucleotide that targets a STMN2 transcript with a cryptic exon and is capable of increasing, restoring, or stabilizing full-length STMN2 activity e.g., full length STMN2 expression, for example, full length STMN2 mRNA and/or full length STMN2 protein expression. STMN2 parent oligonucleotides do not include a spacer. Examples of STMN2 parent oligonucleotides include oligonucleotides comprising a sequence of any one of SEQ ID NOs: 1-446 and SEQ ID NOs: 893-1338. As described hereafter, STMN2 oligonucleotide with spacers and STMN2 oligonucleotide variants are described in relation to a corresponding STMN2 parent oligonucleotide.

The term “STMN2 oligonucleotide variant” refers to a STMN2 oligonucleotide that represents a modified version of a corresponding STMN2 parent oligonucleotide. For example, a STMN2 oligonucleotide variant represents a shortened version of a STMN2 parent oligonucleotide. In various embodiments, a STMN2 oligonucleotide variant is any one of a 15mer, 16mer, 17mer, 18mer 19mer, 20mer, 21mer, 22mer, 23mer, 24mer, 25mer, 26mer, or 27mer. Examples of STMN2 oligonucleotide variants include oligonucleotides comprising a sequence of any one of SEQ ID NOs: 1342-1366 or SEQ ID NOs: 1392-1521. In various embodiments, STMN2 oligonucleotide variants comprise one or more spacers. Such STMN2 oligonucleotide variants comprise a sequence of any one of SEQ ID NOs: 1342-1366 and SEQ ID NOs: 1392-1416.

The term “STMN2 oligonucleotide with one or more spacers” or “STMN2 oligonucleotide comprising a spacer” refers to a STMN2 oligonucleotide with at least one spacer. An oligonucleotide with one or more spacers can, in various embodiments, include one spacer, two spacers, three spacers, four spacer, five spacers, six spacers, seven spacers, eight spacers, nine spacers, or ten spacers. In various embodiments, an oligonucleotide comprising one or more spacers includes at least one segment with at most 7 linked nucleosides. For example, as described in a 5′ to 3′ direction, an oligonucleotide comprising a spacer can include a segment with 7 linked nucleosides, followed by a spacer, a second segment with 9 linked nucleosides, followed by a second spacer, and a third segment with 7 linked nucleosides. Here, the first segment of 7 linked nucleosides and the third segment of 7 linked nucleosides each represents segments with at most 7 linked nucleosides. As another example, an oligonucleotide comprising a spacer can include a segment with 10 linked nucleosides, followed by a spacer, a second segment with 10 linked nucleosides, followed by a second spacer, and a third segment with 3 linked nucleosides. Here, the third segment of 3 linked nucleosides represents the segment with at most 7 linked nucleosides. In various embodiments, an oligonucleotide with one or more spacers includes multiple segments with at most 7 linked nucleosides. In various embodiments, every segment of an oligonucleotide with one or more spacers has at most 7 linked nucleosides. For example, the oligonucleotide may be a 23mer and include two spacers that divide the 23mer into three separate segments of 7 linked nucleosides each. Therefore, each segment of the oligonucleotide has at most 7 linked nucleosides.

Generally, STMN2 oligonucleotides comprising one or more spacers are described in reference to a corresponding STMN2 parent oligonucleotide or a corresponding STMN2 oligonucleotide variant. Example STMN2 oligonucleotides comprising one or spacers include any of SEQ ID NOs: 1417-1420 and SEQ ID NOs: 1451-1664.

The phrase “a STMN2 oligonucleotide that targets a STMN2 transcript” refers to a STMN2 oligonucleotide that binds to a STMN2 transcript. Example regions of a STMN2 transcript are shown in Table 1, which depicts sequences corresponding to regions of branch points (e.g., branch point 1, 2, and 3) a 3′ splice acceptor region, an ESE binding region, TDP43 binding sites, a cryptic exon, and a Poly A region. In various embodiments, the oligonucleotide binds to a region of a STMN2 transcript with a cryptic exon, the region being located less than 75 nucleobases upstream or downstream to any of the branch points (e.g., branch point 1, 2, and 3) a 3′ splice acceptor region, an ESE binding region, TDP43 binding sites, a cryptic exon, and a Poly A region.

### KCNQ2

As used herein, “KCNQ2” (also known as Kv7.2, KCNA11, HNSPC, ENB1, BNFC, Potassium Channel, Voltage Gated KQT-Like Subfamily Q, Member 2, Neuroblastoma-Specific Potassium Channel Subunit Alpha KvLQT2, Potassium Voltage-Gated Channel Subfamily KQT Member 2) refers to the gene or gene products (e.g., protein or mRNA transcript (including pre-mRNA) encoded by the gene) identified by any one of SEQ ID NOs: 3032-3043 and allelic variants thereof, as well as orthologs found in non-human species (e.g., non-human primates or mice).

The term “KCNQ2 transcript” refers to a KCNQ2 transcript. Such a KCNQ2 transcript can be a KCNQ2 pre-mRNA sequence or a KCNQ2 mature RNA sequence. KCNQ2 transcript sequences are shown to contain thymine (T), but one of skill in the art will appreciate that thymine (T) can generally be replaced with uracil (U) in RNA sequences.

The term “KCNQ2 oligonucleotide,” “KCNQ2 antisense oligonucleotide,” or “KCNQ2 AON” refers to an oligonucleotide that is capable of increasing, restoring, or stabilizing full-length KCNQ2 activity e.g., full length KCNQ2 expression, for example, full length KCNQ2 mRNA and/or full length KCNQ2 protein expression. Generally, a KCNQ2 oligonucleotide reduces the level of mis-spliced KCNQ2 transcripts by targeting a KCNQ2 transcript (e.g., KCNQ2 pre-mRNA or mis-spliced KCNQ2 with a target sequence). In various embodiments, a KCNQ2 oligonucleotide comprises a sequence that is at least 85% complementary to an equal length portion of a transcript comprising a sequence at least 90% identity to any one of SEQ ID NOs: 3032-3045 or a contiguous 15 to 50 nucleobase portion to any one of SEQ ID NO: 3032-3043. In various embodiments, a KCNQ2 oligonucleotide comprises a sequence that is between 85 and 98% complementary to an equal length portion of a transcript comprising a sequence at least 90% identity to any one of SEQ ID NOs: 3032-3045, or a contiguous 15 to 50 nucleobase portion to any one of SEQ ID NOs: 3032-3045. KCNQ2 target sequences are shown to contain thymine (T), but one of skill in the art will appreciate that thymine (T) can generally be replaced with uracil (U) in RNA sequences.

In various embodiments, KCNQ2 oligonucleotides are characterized by having one or more spacers, where each spacer divides up the KCNQ2 oligonucleotide into segments of linked nucleosides. In various embodiments, KCNQ2 oligonucleotides have two spacers. In one embodiment, KCNQ2 oligonucleotides have two segments of linked nucleosides separated by one spacer. In one embodiment, KCNQ2 oligonucleotides have three segments of linked nucleosides separated by two spacers. In such embodiments, KCNQ2 oligonucleotides have one segment with at most 7 linked nucleosides. For example, a KCNQ2 oligonucleotide may have, from the 5′ to the 3′ end, 5 linked nucleosides, followed by a spacer, 10 linked nucleosides, followed by a second spacer, and 8 linked nucleosides. Thus, the first segment of 5 linked nucleosides satisfies the one segment with at most 7 linked nucleosides. In various embodiments, KCNQ2 oligonucleotides have three spacers that divide the KCNQ2 oligonucleotide into four segments. In various embodiments, each of the four segments of the KCNQ2 oligonucleotide have at most 7 linked nucleosides.

As used herein, the term “KCNQ2 oligonucleotide” encompasses a “KCNQ2 parent oligonucleotide,” a “KCNQ2 oligonucleotide with one or more spacers” (e.g., KCNQ2 oligonucleotide with two spacers or a KCNQ2 oligonucleotide with three spacers), a “KCNQ2 oligonucleotide variant with one or more spacers.” Examples of KCNQ2 oligonucleotides include oligonucleotides comprising a sequence of any one of SEQ ID NOs: 1676-1851, SEQ ID NOs: 2028-2529, SEQ ID NOs: 3046-3221, SEQ ID NO: 3398-3899, and SEQ ID NOs: 4402-4530.

The term “KCNQ2 parent oligonucleotide” refers to an oligonucleotide that targets a KCNQ2 transcript and is capable of increasing, restoring, or stabilizing full-length KCNQ2 activity e.g., full length KCNQ2 expression, for example, full length KCNQ2 mRNA and/or full length KCNQ2 protein expression. KCNQ2 parent oligonucleotides do not include a spacer. Examples of KCNQ2 parent oligonucleotides include oligonucleotides comprising a sequence of any one of SEQ ID NOs: 1676-1851 and SEQ ID NOs: 2028-2529. As described hereafter, KCNQ2 oligonucleotide with spacers and KCNQ2 oligonucleotide variants are described in relation to a corresponding KCNQ2 parent oligonucleotide.

The term “KCNQ2 oligonucleotide variant” refers to a KCNQ2 oligonucleotide that represents a modified version of a corresponding KCNQ2 parent oligonucleotide. For example, a KCNQ2 oligonucleotide variant represents a shortened version of a KCNQ2 parent oligonucleotide. In various embodiments, a KCNQ2 oligonucleotide variant is any one of a 15mer, 16mer, 17mer, 18mer 19mer, 20mer, 21mer, 22mer, 23mer, 24mer, 25mer, 26mer, or 27mer. Examples of KCNQ2 oligonucleotide variants include oligonucleotides comprising a sequence of any one of SEQ ID NOs: 3046-3221, and SEQ ID NO: 3398-3899. In various embodiments, KCNQ2 oligonucleotide variants comprise one or more spacers. Such KCNQ2 oligonucleotide variants comprise a sequence of any one of SEQ ID NOs: 4469-4476, and SEQ ID NOs: 4477-4530.

Generally, KCNQ2 oligonucleotides comprising one or more spacers are described in reference to a corresponding KCNQ2 parent oligonucleotide or a corresponding KCNQ2 oligonucleotide variant. Example KCNQ2 oligonucleotides comprising one or spacers include any of SEQ ID NOs: 4402-4468, SEQ ID NOs: 4469-4476, and SEQ ID NOs: 4477-4530.

The phrase “a KCNQ2 oligonucleotide that targets a KCNQ2 transcript” refers to a KCNQ2 oligonucleotide that binds to a KCNQ2 transcript.

### UNC13A

As used herein, “UNC13A” (also known as Unc-13 Homolog A, Munc13-1, KIAA1032, unc-13 homolog A (C. elegans), or Protein Unc-13 Homolog A) refers to the gene or gene products (e.g., protein or mRNA transcript (including pre-mRNA) encoded by the gene) identified by Entrez Gene ID No. 23025 and allelic variants thereof, as well as orthologs found in non-human species (e.g., non-human primates or mice). UNC13A transcript sequences are shown to contain thymine (T), but one of skill in the art will appreciate that thymine (T) can generally be replaced with uracil (U) in RNA sequences.

The term “UNC13A transcript” refers to a UNC13A transcript which can be a UNC13A pre-mRNA sequence or a UNC13A mature RNA sequence. The term “UNC13A oligonucleotide,” “UNC13A antisense oligonucleotide,” or “UNC13A AON” refers to an oligonucleotide that is capable of increasing, restoring, or stabilizing full-length UNC13A activity e.g., full length UNC13A expression, for example, full length UNC13A mRNA and/or full length UNC13A protein expression. Generally, a UNC13A oligonucleotide reduces the level of mis-spliced UNC13A transcripts by targeting a UNC13A transcript (e.g., UNC13A pre-mRNA or mis-spliced UNC13A with a target sequence). In various embodiments, a UNC13A oligonucleotide comprises a sequence that is at least 85% complementary to an equal length portion of a transcript comprising a sequence at least 90% identity to SEQ ID NO: 9587-9595, or a contiguous 15 to 50 nucleobase portion of SEQ ID NO: 9587-9595. In various embodiments, a UNC13A oligonucleotide comprises a sequence that is between 85 and 98% complementary to an equal length portion of a transcript comprising a sequence at least 90% identity to any one of SEQ ID NO: 9587-9595, or a contiguous 15 to 50 nucleobase portion to any one of SEQ ID NO: 9587-9595. UNC13A target sequences are shown to contain thymine (T), but one of skill in the art will appreciate that thymine (T) can generally be replaced with uracil (U) in RNA sequences.

In various embodiments, UNC13A oligonucleotides are characterized by having one or more spacers, where each spacer divides up the UNC13A oligonucleotide into segments of linked nucleosides. In various embodiments, UNC13A oligonucleotides have two spacers. In one embodiment, UNC13A oligonucleotides have two segments of linked nucleosides separated by one spacer. In one embodiment, UNC13A oligonucleotides have three segments of linked nucleosides separated by two spacers. In such embodiments, UNC13A oligonucleotides have one segment with at most 7 linked nucleosides. For example, a UNC13A oligonucleotide may have, from the 5′ to the 3′ end, 5 linked nucleosides, followed by a spacer, 10 linked nucleosides, followed by a second spacer, and 8 linked nucleosides. Thus, the first segment of 5 linked nucleosides satisfies the one segment with at most 7 linked nucleosides. In various embodiments, UNC13A oligonucleotides have three spacers that divide the UNC13A oligonucleotide into four segments. In various embodiments, each of the four segments of the UNC13A oligonucleotide have at most 7 linked nucleosides.

As used herein, the term “UNC13A oligonucleotide” encompasses a “UNC13A parent oligonucleotide,” a “UNC13A oligonucleotide with one or more spacers” (e.g., UNC13A oligonucleotide with two spacers or a UNC13A oligonucleotide with three spacers), a “UNC13A oligonucleotide variant with one or more spacers.” Examples of UNC13A oligonucleotides include oligonucleotides comprising a sequence of any one of SEQ ID NOs: 4531-5794, SEQ ID NOs: 7059-8322, SEQ ID NOs: 9596-9696, and SEQ ID NOs: 10670-10779.

The term “UNC13A parent oligonucleotide” refers to an oligonucleotide that targets a UNC13A transcript and is capable of increasing, restoring, or stabilizing full-length UNC13A activity e.g., full length UNC13A expression, for example, full length UNC13A mRNA and/or full length UNC13A protein expression. UNC13A parent oligonucleotides do not include a spacer. Examples of UNC13A parent oligonucleotides include oligonucleotides comprising a sequence of any one of SEQ ID NO: 4531-5794. As described hereafter, UNC13A oligonucleotide with spacers and UNC13A oligonucleotide variants are described in relation to a corresponding UNC13A parent oligonucleotide.

The term “UNC13A oligonucleotide variant” refers to a UNC13A oligonucleotide that represents a modified version of a corresponding UNC13A parent oligonucleotide. For example, a UNC13A oligonucleotide variant represents a shortened version of a UNC13A parent oligonucleotide. In various embodiments, a UNC13A oligonucleotide variant is any one of a 15mer, 16mer, 17mer, 18mer 19mer, 20mer, 21mer, 22mer 23mer, or 24mer. Examples of UNC13A oligonucleotide variants include oligonucleotides comprising a sequence of any one of SEQ ID NO: 7059-8322. In various embodiments, UNC13A oligonucleotide variants comprise one or more spacers. Such UNC13A oligonucleotide variants comprise a sequence of any one of SEQ ID NO: 9596-9696.

Generally, UNC13A oligonucleotides comprising one or more spacers are described in reference to a corresponding UNC13A parent oligonucleotide or a corresponding UNC13A oligonucleotide variant. Example UNC13A oligonucleotides comprising one or spacers include any of SEQ ID NOs: 9596-9696.

The phrase “a UNC13A oligonucleotide that targets a UNC13A transcript” refers to a UNC13A oligonucleotide that binds to a UNC13A transcript

### SMN2

As used herein, “SMN2” (also known as Survival of Motor Neuron 2, SMNC, TRD16B, BCD541, GEMINI1, Survival Motor Neuron Protein, Tudor Domain Containing 16B, Component of Gems 1, Gemin-1, C-BCD541, SMNT, SMN) refers to the gene or gene products (e.g., protein or mRNA transcript (including pre-mRNA) encoded by the gene) identified by any one of SEQ ID NOs: 9698-9709 and allelic variants thereof, as well as orthologs found in non-human species (e.g., non-human primates or mice).

The term “SMN2 transcript” refers to a SMN2 transcript. Such a SMN2 transcript can be a SMN2 pre-mRNA sequence or a SMN2 mature RNA sequence. SMN2 transcript sequences are shown to contain thymine (T), but one of skill in the art will appreciate that thymine (T) can generally be replaced with uracil (U) in RNA sequences.

The term “SMN2 oligonucleotide,” “SMN2 antisense oligonucleotide,” or “SMN2 AON” refers to an oligonucleotide that is capable of increasing, restoring, or stabilizing full-length SMN2 activity e.g., full length SMN2 expression, for example, full length SMN2 mRNA and/or full length SMN2 protein expression. Generally, a SMN2 oligonucleotide reduces the level of mis-spliced SMN2 transcripts by targeting a SMN2 transcript (e.g., SMN2 pre-mRNA or mis-spliced SMN2 with a target sequence). In various embodiments, a SMN2 oligonucleotide comprises a sequence that is at least 85% complementary to an equal length portion of a transcript comprising a sequence at least 90% identity to any one of SEQ ID NOs: 9698-9709 or a contiguous 15 to 50 nucleobase portion to any one of SEQ ID NO: 9698-9709. In various embodiments, a SMN2 oligonucleotide comprises a sequence that is between 85 and 98% complementary to an equal length portion of a transcript comprising a sequence at least 90% identity to any one of SEQ ID NOs: 9698-9709, or a contiguous 15 to 50 nucleobase portion to any one of SEQ ID NOs: 9698-9709. SMN2 target sequences are shown to contain thymine (T), but one of skill in the art will appreciate that thymine (T) can generally be replaced with uracil (U) in RNA sequences.

In various embodiments, SMN2 oligonucleotides are characterized by having one or more spacers, where each spacer divides up the SMN2 oligonucleotide into segments of linked nucleosides. In various embodiments, SMN2 oligonucleotides have two spacers. In one embodiment, SMN2 oligonucleotides have two segments of linked nucleosides separated by one spacer. In one embodiment, SMN2 oligonucleotides have three segments of linked nucleosides separated by two spacers. In such embodiments, SMN2 oligonucleotides have one segment with at most 7 linked nucleosides. For example, a SMN2 oligonucleotide may have, from the 5′ to the 3′ end, 5 linked nucleosides, followed by a spacer, 10 linked nucleosides, followed by a second spacer, and 8 linked nucleosides. Thus, the first segment of 5 linked nucleosides satisfies the one segment with at most 7 linked nucleosides. In various embodiments, SMN2 oligonucleotides have three spacers that divide the SMN2 oligonucleotide into four segments. In various embodiments, each of the four segments of the SMN2 oligonucleotide have at most 7 linked nucleosides.

As used herein, the term “SMN2 oligonucleotide” encompasses a “SMN2 parent oligonucleotide,” a “SMN2 oligonucleotide with one or more spacers” (e.g., SMN2 oligonucleotide with two spacers or a SMN2 oligonucleotide with three spacers), a “SMN2 oligonucleotide variant with one or more spacers.” Examples of SMN2 oligonucleotides include oligonucleotides comprising a sequence of any one of SEQ ID NOs: 9710-10141, SEQ ID NOs: 10574-10651, and SEQ ID NOs: 10783-10808.

The term “SMN2 parent oligonucleotide” refers to an oligonucleotide that targets a SMN2 transcript and is capable of increasing, restoring, or stabilizing full-length SMN2 activity e.g., full length SMN2 expression, for example, full length SMN2 mRNA and/or full length SMN2 protein expression. SMN2 parent oligonucleotides do not include a spacer. As described hereafter, SMN2 oligonucleotide with spacers and SMN2 oligonucleotide variants are described in relation to a corresponding SMN2 parent oligonucleotide.

The term “SMN2 oligonucleotide variant” refers to a SMN2 oligonucleotide that represents a modified version of a corresponding SMN2 parent oligonucleotide. For example, a SMN2 oligonucleotide variant represents a shortened version of a SMN2 parent oligonucleotide. In various embodiments, a SMN2 oligonucleotide variant is any one of a 15mer, 16mer, 17mer, 18mer 19mer, 20mer, 21mer, 22mer, 23mer, or 24mer. Examples of SMN2 oligonucleotide variants include oligonucleotides comprising a sequence of any one of SEQ ID NOs: 9710-10141. In various embodiments, SMN2 oligonucleotide variants comprise one or more spacers. In various embodiments, SMN2 oligonucleotide variants may comprise a sequence of any one of SEQ ID NOs: 10574-10643 and SEQ ID NOs: 10644-10651.

Generally, SMN2 oligonucleotides comprising one or more spacers are described in reference to a corresponding SMN2 parent oligonucleotide or a corresponding SMN2 oligonucleotide variant. Example SMN2 oligonucleotides comprising one or spacers include any of SEQ ID NOs: 10574-10640 and SEQ ID NOs: 10644-10651.

In various embodiments, one or more spacers may be located at one or more positions of an oligonucleotide. A spacer may be located between a first position and a second position of the oligonucleotide. As used herein, a spacer located between a first position and second position encompasses the spacer being located at the first position, located at the second position, or located at any position of the oligonucleotide sandwiched by the first position and the second position.

In the present specification, the term “therapeutically effective amount” means the amount of an oligonucleotide that will elicit the biological or medical response of a tissue, system, animal or human that is being sought by the researcher, veterinarian, medical doctor, or other clinician. In some embodiments, the oligonucleotide comprises a sequence that is at least 85% complementary to an equal length portion of a transcript comprising a sequence at least 90% identity to SEQ ID NO: 1339 or SEQ ID NO: 1341, or a contiguous 15 to 50 nucleobase portion of SEQ ID NO: 1339 or SEQ ID NO: 1341. In some embodiments, the oligonucleotide comprises a sequence that is at least 85% complementary to an equal length portion of a transcript comprising a sequence at least 90% identity to a sequence at least 90% identity to any one of SEQ ID NO: 3032-3045, or a contiguous 15 to 50 nucleobase portion to any one of SEQ ID NO: 3032-3045. In some embodiments, the oligonucleotide comprises a sequence that is at least 85% complementary to an equal length portion of a transcript comprising a sequence at least 90% identity to a sequence that is at least 85% complementary to an equal length portion of a transcript comprising a sequence at least 90% identity to SEQ ID NO: 9587-9595, or a contiguous 15 to 50 nucleobase portion of SEQ ID NO: 9587-9595. In some embodiments, the oligonucleotide comprises a sequence that is at least 85% complementary to an equal length portion of a transcript comprising a sequence at least 90% identity to any one of SEQ ID NO: 9698-9709, or a contiguous 15 to 50 nucleobase portion to any one of SEQ ID NO: 9698-9709. The oligonucleotide is administered in therapeutically effective amounts to treat and/or prevent a disease, condition, disorder, or state, for example, a neurological disease and/or a neuropathy. Alternatively, a therapeutically effective amount of an oligonucleotide is the quantity required to achieve a desired therapeutic and/or prophylactic effect, such as an amount which results in the prevention of or a decrease in the symptoms associated with a disease associated with reduced STMN2 activity in the motor neurons, an amount which results in the prevention of or a decrease in the symptoms associated with a disease associated with reduced KCNQ2 activity in the motor neurons, an amount which results in the prevention of or a decrease in the symptoms associated with a disease associated with reduced UNC13A activity in the motor neurons, and/or an amount which results in the prevention of or a decrease in the symptoms associated with a disease associated with reduced SMN2 activity in the motor neurons.

The term “pharmaceutically acceptable salt(s)” as used herein refers to salts of acidic or basic groups that may be present in a STMN2 oligonucleotide used in the present compositions, a KCNQ2 oligonucleotide used in the present compositions, a UNC13A oligonucleotide used in the present compositions, and/or a SMN2 oligonucleotide used in the present compositions. A STMN2 oligonucleotide, KCNQ2 oligonucleotide, UNC13A oligonucleotide, and/or SMN2 oligonucleotide included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. A STMN2 oligonucleotide, KCNQ2 oligonucleotide, UNC13A oligonucleotide, and/or SMN2 oligonucleotide included in the present compositions that include an amino moiety may form pharmaceutically acceptable salts with various amino acids, in addition to the acids mentioned above. Compounds included in the present compositions that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts. Pharmaceutically acceptable salts of the disclosure include, for example, pharmaceutically acceptable salts of STMN2 oligonucleotides that include a sequence of any of SEQ ID NOs: 1-466, SEQ ID NO: 893-1338, SEQ ID NOs: 1342-1366, SEQ ID NOs: 1392-1664, and SEQ ID NOs: 10655-10669; pharmaceutically acceptable salts of KCNQ2 oligonucleotides that include a sequence of any of SEQ ID NOs: 1676-1851, SEQ ID NOs: 2028-2529, SEQ ID NOs: 3046-3221, SEQ ID NO: 3398-3899, and SEQ ID NOs: 4402-4530; pharmaceutically acceptable salts of UNC13A oligonucleotides that include a sequence of any of SEQ ID NOs: 4531-5794, SEQ ID NOs: 7059-8322, SEQ ID NOs: 9596-9696, and SEQ ID NOs: 10670-10779; and pharmaceutically acceptable salts of SMN2 oligonucleotides that include a sequence of any of SEQ ID NOs: 9710-10141 and SEQ ID NOs: 10574-10651.

A STMN2 oligonucleotide, KCNQ2 oligonucleotide, UNC13A oligonucleotide, and/or SMN2 oligonucleotide of the disclosure may contain one or more chiral centers, groups, linkages, and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S” (or “Rp” or “Sp”) depending on the configuration of substituents around the stereogenic atom, for example, a stereogenic carbon, phosphorous, or sulfur atom. In some embodiments, one or more linkages of the compound may have a Rp or Sp configuration (e.g., one or more phosphorothioate linkages have either a Rp or Sp configuration). The configuration of each phosphorothioate linkage may be independent of another phosphorothioate linkage (e.g., one phosphorothioate linkage has a Rp configuration and a second phosphorothioate linkage has a Sp configuration). In various embodiments, the STMN2 oligonucleotide, KCNQ2 oligonucleotide, UNC13A oligonucleotide, and/or SMN2 oligonucleotide can have a mixed configuration of phosphorothioate linkages. For example, the STMN2 oligonucleotide, KCNQ2 oligonucleotide, UNC13A oligonucleotide, and/or SMN2 oligonucleotide may have five phosphorothioate linkages in a Rp configuration, followed by fifteen phosphorothioate linkages in a Sp configuration, followed by five phosphorothioate linkages in a Rp configuration. The present invention encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly.

Individual stereoisomers of a STMN2 oligonucleotide, KCNQ2 oligonucleotide, UNC13A oligonucleotide, and/or SMN2 oligonucleotide of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well-known methods, such as chiral-phase gas chromatography, chiral-phase super critical fluid chromatography, chiral-phase simulated moving bed chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well-known asymmetric synthetic methods.

The STMN2 oligonucleotide, KCNQ2 oligonucleotide, UNC13A oligonucleotide, and/or SMN2 oligonucleotide disclosed herein can exist in solvated as well as unsolvated forms with pharmaceutically acceptable solvents such as water, ethanol, and the like, and it is intended that the invention embrace both solvated and unsolvated forms.

The disclosure also embraces fluorescently labeled compounds of the invention. The disclosure also embraces isotopically labeled compounds of the invention (i.e., isotopically labeled STMN2 oligonucleotide, KCNQ2 oligonucleotide, UNC13A oligonucleotide, and/or SMN2 oligonucleotide) which are identical to those recited herein, except that one or more atoms are replaced by an atom having an atomic mass or mass number different from the atomic mass or mass number abundantly found in nature. Examples of isotopes that can be incorporated into compounds of the invention include isotopes of hydrogen, carbon, nitrogen, oxygen, phosphorus, fluorine and chlorine, such as ²H, ³H, ¹¹C, ¹³C, ¹⁴C, ¹⁵N, ¹⁸O, ¹⁷O, ³¹P, ³²P, ³³P, ³⁵S, ¹⁸F, and ³⁶Cl, respectively.

Certain isotopically labeled disclosed compounds (e.g., those labeled with ³H, ¹⁴C, or ³⁵S) are useful in compound and/or substrate tissue distribution assays. Tritiated (i.e., ³H), carbon-14 (i.e., ¹⁴C), or ³⁵S isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances.

As used herein, “2′-O-(2-methoxyethyl)” (also 2′-MOE and 2′-O(CH₂)₂OCH₃ and MOE) refers to an O-methoxyethyl modification of the 2′ position of a furanose ring. A 2′-O-(2-methoxyethyl) is used interchangeably as “2′-O-methoxyethyl” in the present disclosure. A sugar moiety in a nucleoside modified with 2′-MOE is a modified sugar.

As used herein, “2′-MOE nucleoside” (also 2′-O-(2-methoxyethyl) nucleoside) means a nucleoside comprising a 2′-MOE modified sugar moiety.

As used herein, “2′-substituted nucleoside” means a nucleoside comprising a substituent at the 2′-position of the furanose ring other than H or OH. In certain embodiments, 2′ substituted nucleosides include nucleosides with bicyclic sugar modifications.

As used herein, “5-methyl cytosine” (5-MeC) means a cytosine modified with a methyl group attached to the 5 position. A 5-methyl cytosine (5-MeC) is a modified nucleobase.

As used herein, “bicyclic sugar” means a furanose ring modified by the bridging of two atoms. A bicyclic sugar is a modified sugar.

As used herein, “bicyclic nucleoside” (also BNA) means a nucleoside having a sugar moiety comprising a bridge connecting two carbon atoms of the sugar ring, thereby forming a bicyclic ring system. In certain embodiments, the bridge connects the 4′-carbon and the 2′-carbon of the sugar ring.

As used herein, “cap structure” or “terminal cap moiety” means chemical modifications, which have been incorporated at either terminus of an antisense compound.

As used herein, “cEt” or “constrained ethyl” means a bicyclic nucleoside having a sugar moiety comprising a bridge connecting the 4′-carbon and the 2′-carbon, wherein the bridge has the formula: 4′-CH(CH₃)—O-2′.

As used herein, “constrained ethyl nucleoside” (also cEt nucleoside) means a nucleoside comprising a bicyclic sugar moiety comprising a 4′-CH(CH₃)—O-2′ bridge. In some embodiments, cEt can be modified. In some embodiments, the cEt can be S-cEt (in an S-constrained ethyl 2′-4′-bridged nucleic acid). In some other embodiments, the cEt can be R-cEt.

As used herein, “internucleoside linkage” refers to the covalent linkage between adjacent nucleosides in an oligonucleotide. In some embodiments, as used herein, “non-natural linkage” refers to a “modified internucleoside linkage.”

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence. As an example to the contrary, two nucleosides separated by a spacer are not contiguous.

As used herein, “locked nucleic acid” or “LNA” or “LNA nucleosides” means nucleic acid monomers having a bridge (e.g., methylene, ethylene, aminooxy, or oxyimino bridge) connecting two carbon atoms between the 4′ and 2′ position of the nucleoside sugar unit, thereby forming a bicyclic sugar. Examples of such bicyclic sugar include, but are not limited to (A) α-L-Methyleneoxy (4′-CH₂—O-2′) LNA, (B) β-D-Methyleneoxy (4′-CH₂—O-2′) LNA, (C) Ethyleneoxy (4′-(CH₂)₂—O-2′) LNA, (D) Aminooxy (4′-CH₂—O—N(R)-2′) LNA and (E) Oxyamino (4′-CH₂—N(R)—O-2′) LNA; wherein R is H, C₁-C₁₂ alkyl, or a protecting group (see U.S. Pat. No. 7,427,672, issued on Sep. 23, 2008).

As used herein, LNA compounds include, but are not limited to, compounds having at least one bridge between the 4′ and the 2′ position of the sugar wherein each of the bridges independently comprises 1 or from 2 to 4 linked groups independently selected from —[C(R₁)(R₂)]ₙ—, —C(R₁)═C(R₂)—, —C(R₁)═N—, —C(═NR₁)—, —C(═O)—, —C(═S)—, —O—, —Si(R₁)₂—, —S(═O)ₓ— and —N(R₁)—; wherein: x is 0, 1, or 2; n is 1, 2, 3, or 4; each R₁ and R₂ is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, a heterocycle radical, a substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

Examples of 4′-2′ bridging groups encompassed within the definition of LNA include, but are not limited to one of formulae: —[C(R₁)(R₂)]ₙ—, —[C(R₁)(R₂)]ₙ—O—, —C(R₁R₂)—N(R₁)—O— or —C(R₁R₂)—O—N(R₁)—. Furthermore, other bridging groups encompassed with the definition of LNA are 4′-CH₂-2′, 4′-(CH₂)₂-2′, 4′-(CH₂)₃-2′, 4′—CH₂—O-2′, 4′-(CH₂)₂—O-2′, 4′-CH₂—O—N(R₁)-2′ and 4′-CH₂—N(R₁)—O-2′-bridges, wherein each R₁ and R₂ is, independently, H, a protecting group or C₁-C₁₂ alkyl.

Also included within the definition of LNA according to the invention are LNAs in which the 2′-hydroxyl group of the ribosyl sugar ring is connected to the 4′ carbon atom of the sugar ring, thereby forming a bridge to form the bicyclic sugar moiety. The bridge can be a methylene (—CH₂—) group connecting the 2′ oxygen atom and the 4′ carbon atom, for which the term methyleneoxy (4′-CH₂—O-2′) LNA is used. Furthermore, in the case of the bicyclic sugar moiety having an ethylene bridging group in this position, the term ethyleneoxy (4′-CH₂CH₂—O-2′) LNA is used.

As used herein, a “spacer” refers to a nucleoside-replacement group (e.g., a non-nucleoside group that replaces a nucleoside present in a STMN2 parent oligonucleotide, KCNQ2 parent oligonucleotide, UNC13A parent oligonucleotide, and/or SMN2 parent oligonucleotide). The spacer is characterized by the lack of a nucleotide base and by the replacement of the nucleoside sugar moiety with a non-sugar substitute. The non-sugar substitute group of a spacer lacks an aldehyde, ketone, acetal, ketal, hemiacetal or hemiketal group. The non-sugar substitute group of a spacer is thus capable of connecting to the 3′ and 5′ positions of the nucleosides adjacent to the spacer through an internucleoside linker as described herein, but not capable of forming a covalent bond with a nucleotide base (i.e., not capable of linking a nucleobase to another group, such as an internucleoside linkage, conjugate group, or terminal group in an oligonucleotide). Generally, a STMN2 oligonucleotide with a spacer is described in relation to a STMN2 parent oligonucleotide, wherein the spacer replaces a nucleoside of the STMN2 parent oligonucleotide. Generally, a KCNQ2 oligonucleotide with a spacer is described in relation to a KCNQ2 parent oligonucleotide, wherein the spacer replaces a nucleoside of the KCNQ2 parent oligonucleotide. Generally, a UNC13A oligonucleotide with a spacer is described in relation to a UNC13A parent oligonucleotide, wherein the spacer replaces a nucleoside of the UNC13A parent oligonucleotide. Generally, a SMN2 oligonucleotide with a spacer is described in relation to a SMN2 parent oligonucleotide, wherein the spacer replaces a nucleoside of the SMN2 parent oligonucleotide. In all embodiments of the present disclosure, a spacer cannot hybridize to a nucleoside comprising a nucleobase at the corresponding position of a STMN2 transcript, KCNQ2 transcript, UNC13A transcript, and/or SMN2 transcript, within the numerical order of the length of the AON oligonucleotide (i.e., if the spacer is positioned after nucleoside 4 of an AON (i.e., at position 5 from the 5′-end), the spacer is not complementary to the nucleoside (A, C, G, or U) at the same corresponding position of the target STMN2 transcript, KCNQ2 transcript, UNC13A transcript, and/or SMN2 transcript).

As used herein, “mismatch” or a “non-complementary group” refers to the case when a group (e.g., nucleobase) of a first nucleic acid is not capable of pairing with the corresponding group (e.g., nucleobase) of a second or target nucleic acid.

As used herein, “modified internucleoside linkage” refers to a substitution or any change from a naturally occurring internucleoside linkage (e.g., a phosphodiester internucleoside bond).

As used herein, “modified nucleobase” means any nucleobase other than adenine, cytosine, guanine, thymine, or uracil. Examples of a modified nucleobase include 5-methyl cytosine, pseudouridine, or 5-methoxyuridine. An “unmodified nucleobase” means the purine bases adenine (A) and guanine (G), and the pyrimidine bases thymine (T), cytosine (C), and uracil (U).

As used herein, a “modified nucleoside” means a nucleoside having, independently, a modified sugar moiety and/or modified nucleobase. A universal base is a modified nucleobase that can pair with any one of the five unmodified nucleobases. Modified nucleosides include abasic nucleosides, which lack a nucleobase. However, modified nucleosides do not include spacers or other groups that are incapable of linking a nucleobase.

As used herein, “linked nucleosides” are nucleosides that are connected in a contiguous sequence (i.e., no additional nucleosides are presented between those that are linked). In various embodiments, an oligonucleotide may have different segments of linked nucleosides connected through a spacer. Here, the spacer (i.e., nucleoside replacement) is not considered a nucleoside and therefore, divides up the oligonucleotide into two segments of linked nucleosides. The oligonucleotide may have a first segment of Y linked nucleosides (e.g., Y nucleosides that are connected in a contiguous sequence), followed by a spacer, and then a second segment of Z linked nucleosides. Here, the Y and Z linked nucleosides is described in either the 5′ to 3′ direction or the 3′ to 5′ direction. In various embodiments, the first segment consists of 7 or fewer linked nucleosides (e.g., Y=7 or fewer) whereas the second segment comprises 8 or more linked nucleosides (e.g., Z=8 or more).

As used herein, “modified oligonucleotide” means an oligonucleotide comprising at least one (i.e., one or more) modified internucleoside linkage, modified sugar, and/or modified nucleobase.

As used herein, “modified sugar” or “modified sugar moiety” means a modified furanosyl sugar moiety or a modified sugar moiety having other than a furanosyl moiety that can link a nucleobase to another group, such as an internucleoside linkage, conjugate group, or terminal group in an oligonucleotide.

As used herein, “monomer” means a single unit of an oligomer. Monomers include, but are not limited to, nucleosides and nucleotides, whether naturally occurring or modified.

As used herein, “motif” means the pattern of unmodified and modified nucleosides in an antisense compound.

As used herein, “natural sugar moiety” means a sugar moiety found in DNA (2′-H) or RNA (2′-OH).

As used herein, “naturally occurring internucleoside linkage” means a 3′ to 5′ phosphodiester linkage.

As used herein, “non-complementary nucleobases” refers to a pair of nucleobases that do not form hydrogen bonds with one another or otherwise support hybridization.

As used herein, “nucleic acid” refers to molecules composed of monomeric nucleotides. A nucleic acid includes, but is not limited to, ribonucleic acids (RNA), deoxyribonucleic acids (DNA), single-stranded nucleic acids, double-stranded nucleic acids, non-coding RNA, small interfering ribonucleic acids (siRNA), short-hairpin RNA (shRNA), and microRNAs (miRNA).

As used herein, “nucleobase” means a heterocyclic moiety capable of base pairing with a base of another nucleic acid.

As used herein, “nucleobase complementarity” refers to a nucleobase that is capable of base pairing with another nucleobase. For example, in DNA, adenine (A) is complementary to thymine (T). For example, in RNA, adenine (A) is complementary to uracil (U). In certain embodiments, complementary nucleobase refers to a nucleobase of an antisense compound that is capable of base pairing with a corresponding nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair.

As used herein, “nucleobase sequence” means the order of nucleobases independent of any sugar, linkage, and/or nucleobase modification.

As used herein, “nucleoside” refers to a nucleobase linked to a sugar. The term “nucleoside” also includes a “modified nucleoside” which has independently, a modified sugar moiety and/or modified nucleobase.

As used herein, “nucleoside mimetic” includes those structures used to replace the sugar, the sugar and the base, or the base and not necessarily the linkage at one or more positions of an oligomeric compound such as for example nucleoside mimetics having morpholino, cyclohexenyl, cyclohexyl, tetrahydropyranyl, bicyclo, or tricyclo sugar mimetics, e.g., non-furanose sugar units. Nucleotide mimetic includes those structures used to replace the nucleoside and the linkage at one or more positions of an oligomeric compound such as for example peptide nucleic acids or morpholinos (morpholinos linked by a phosphorodiamidate or other non-phosphodiester linkage). Sugar surrogate overlaps with the slightly broader term nucleoside mimetic but is intended to indicate replacement of the sugar unit (furanose ring) only. The tetrahydropyranyl rings provided herein are illustrative of an example of a sugar surrogate wherein the furanose sugar group has been replaced with a tetrahydropyranyl ring system. “Mimetic” refers to groups that are substituted for a sugar, a nucleobase, and/or internucleoside linkage. Generally, a mimetic is used in place of the sugar or sugar-internucleoside linkage combination, and the nucleobase is maintained for hybridization to a selected target.

As used herein, “nucleotide” means a nucleoside having a phosphate group covalently linked to the sugar portion of the nucleoside.

As used herein, “oligomeric compound” or “oligomer” means a polymer of linked monomeric subunits which is capable of hybridizing to at least a region of a nucleic acid molecule.

As used herein, “oligonucleotide” means a polymer of one or more segments of linked nucleosides each of which can be modified or unmodified, independent one from another.

As used herein, “hotspot region” is a range of nucleobases on a target nucleic acid amenable to oligomeric compound-mediated modulation of the splicing of the target nucleic acid.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding between complementary nucleobases.

As used herein, “increasing the amount of activity” refers to more transcriptional expression, more accurate splicing resulting in full length mature mRNA and/or protein expression, and/or more activity relative to the transcriptional expression or activity in an untreated or control sample.

### Antisense Therapeutics

Antisense therapeutics are a class of nucleic acid-based compounds that can be used to modulate a transcript, such as mRNA. In various embodiments, antisense therapeutics comprise one or more spacers and can be used to modulate a transcript that is transcribed from a gene, such as a STMN2 pre-mRNA comprising a cryptic exon, a KCNQ2 pre-mRNA, a UNC13A pre-mRNA, and/or a SMN2 pre-mRNA.

Antisense therapeutics may be single- or double-stranded deoxyribonucleic acid (DNA)-based, ribonucleic acid (RNA)-based, or DNA/RNA chemical analogue compounds. In general, antisense therapeutics are designed to include a sequence that is complementary or nearly complementary to an mRNA or pre-mRNA sequence transcribed from a given gene in order to promote binding between the antisense therapeutic and the pre-mRNA or mRNA. In certain embodiments, antisense therapeutics act by binding to an mRNA or pre-mRNA, thereby inhibiting protein translation, altering pre-mRNA splicing into mature mRNA (e.g., by preventing appropriate proteins such as splicing activator proteins from binding), and/or causing destruction of mRNA. In certain embodiments, the antisense therapeutic sequence is complementary to a portion of a targeted gene's or mRNA's sense sequence. In certain embodiments, antisense therapeutics described herein are oligonucleotide-based compounds that include an oligonucleotide sequence complementary to a pre-mRNA sense, or a portion thereof, and one or more spacers. In certain embodiments, antisense therapeutics described herein can also be nucleotide chemical analog-based compounds.

In certain embodiments, an oligonucleotide, such as disclosed herein, may be an oligonucleotide sequence of 5 to 100 oligonucleotide units in length, for example, 10 to 60 oligonucleotide units in length, for example, 12 to 50 oligonucleotide units in length, 14 to 40 oligonucleotide units in length, 10 to 30 oligonucleotide units in length, for example, 14 to 30 oligonucleotide units in length, for example, 14 to 25 or 15 to 22 oligonucleotide units in length, or 18, 19, 20, 21, 22, 23, 24, 25, 26, or 27 oligonucleotide units in length. As used herein, an “oligonucleotide unit” refers to either a nucleoside (e.g., a nucleoside which includes a sugar and/or a nucleobase) or a nucleoside-replacement group (e.g., a spacer) of the oligonucleotide.

In particular embodiments, the oligonucleotides are 25 oligonucleotide units in length. In particular embodiments, the oligonucleotides are 23 oligonucleotide units in length. In particular embodiments, the oligonucleotides are 21 oligonucleotide units in length. In particular embodiments, the oligonucleotides are 19 oligonucleotide units in length. In various embodiments, the oligonucleotide is at least 18, at least 19, at least 20, at least 21, at least 22, at least 23, at least 24, or at least 25 oligonucleotide units in length. In various embodiments, the oligonucleotide is at least 18 oligonucleotide units in length. In various embodiments, the oligonucleotide is at least 19 oligonucleotide units in length. In various embodiments, the oligonucleotide is at least 20 oligonucleotide units in length. In various embodiments, the oligonucleotide is at least 21 oligonucleotide units in length. In various embodiments, the oligonucleotide is at least 22 oligonucleotide units in length. In various embodiments, the oligonucleotide is at least 23 oligonucleotide units in length. In various embodiments, the oligonucleotide is at least 24 oligonucleotide units in length. In various embodiments, the oligonucleotide is at least 25 oligonucleotide units in length. In various embodiments, the oligonucleotide is at least 26 oligonucleotide units in length. In various embodiments, the oligonucleotide is at least 27 oligonucleotide units in length.

In certain embodiments, AONs may include chemically modified nucleosides (for example, 2′-O-methylated nucleosides or 2′-O-(2-methoxyethyl) nucleosides) as well as modified internucleoside linkages (for example, phosphorothioate linkages). In certain embodiments, AONs described herein include oligonucleotide sequences that are complementary to RNA sequences, such as STMN2 mRNA sequences. In certain embodiments, AONs described herein can include chemically modified nucleosides and modified internucleoside linkages (for example, phosphorothioate linkages). In particular embodiments, AONs described herein include one or more spacers.

In various embodiments, the oligonucleotides comprise one or more spacers. In particular embodiments, the oligonucleotides comprise one spacer. In various embodiments, the oligonucleotides comprise two spacers. For example, the oligonucleotide includes 23 oligonucleotide units with 21 nucleobases and two nucleoside replacement groups (e.g., two spacers). Further embodiments of oligonucleotides with one spacer and oligonucleotides with two spacers are described herein. In various embodiments, the oligonucleotides comprise three spacers.

In some embodiments, an antisense oligonucleotide can be, but is not limited to, inhibitors of a gene transcript (for example, shRNAs, siRNAs, PNAs, LNAs, 2′-O-methyl (2′OMe) antisense oligonucleotide (AON), 2′-O-(2-methoxyethyl) (MOE) AON, or morpholino oligomers (e.g., phosphorodiamidate morpholino (PMO))), or compositions that include such compounds. In some embodiments an oligonucleotide is an antisense oligonucleotide (AON) comprising 2′OMe (e.g., a AON comprising one or more 2′OMe modified sugar), MOE (e.g., a AON comprising one or more MOE modified sugar), peptide nucleic acids (e.g., a AON comprising one or more N-(2-aminoethyl)-glycine units linked by amide bonds or carbonyl methylene linkage as repeating units in place of a sugar-phosphate backbone), locked nucleic acids (e.g., a AON comprising one or more locked ribose, and can be a mixture of 2′-deoxy nucleotides or 2′OMe nucleotides), c-ET (e.g., a AON comprising one or more cET sugar), constrained methoxyethyl (cMOE) (e.g., a AON comprising one or more cMOE sugar), morpholino oligomer (e.g., a AON comprising a backbone comprising one or more PMO), deoxy-2′-fluoro nucleoside (e.g., a AON comprising one or more 2′-fluoro-β-D-arabinonucleoside), tricyclo-DNAs (tcDNA) (e.g., a AON comprising one or more tcDNA modified sugar), 2′-O,4′-C-Ethylene-bridged nucleic acid (ENA) (e.g., a AON comprising one or more ENA modified sugar), or hexitol nucleic acids (HNA) (e.g., a AON comprising one or more HNA modified sugar). In some embodiments, a AON comprises one or more internucleoside linkage independently selected from a phosphorothioate linkage, phosphodiester linkage, phosphotriester linkage, methylphosphonate linkage, phosphoramidate linkage, a phosphoramidothioate linkage, a thiophosphorodiamidate linkage, phosphorodiamidate morpholino (PMO) (morpholino) linkage, PNA linkage, or any combination of phosphorothioate linkage, phosphodiester linkage, a phosphotriester linkage, methylphosphonate linkage, phosphoramidate linkage, a phosphoramidothioate linkage, thiophosphorodiamidate linkage, phosphorodiamidate morpholino (PMO) (morpholino) linkage, and PNA linkage. In some embodiments, a STMN2 AON, a KCNQ2 AON a UNC13A AON, or a SMN2 AON comprises one or more phosphorothioate linkage, phosphodiester linkage, or a combination of phosphorothioate and phosphodiester linkages.

Peptide nucleic acids (PNAs) are short, artificially synthesized polymers with a structure that mimics DNA or RNA. PNAs include a backbone composed of repeating N-(2-aminoethyl)-glycine units linked by peptide bonds. In certain embodiments, PNAs described herein can be used as antisense therapeutics that bind to RNA sequences with high specificity and increase, restore, and/or stabilize levels (e.g., full length UNC13A mRNA or protein levels) and/or activity (e.g., biological activity, for example, UNC13A activity).

Locked nucleic acids (LNAs) are oligonucleotide sequences that include one or more modified RNA nucleotides in which the ribose moiety is modified with an extra bridge connecting the 2′ oxygen and 4′ carbon. LNAs are believed to have higher Tm's than analogous oligonucleotide sequences. In certain embodiments, LNAs described herein can be used as antisense therapeutics that bind to RNA sequences with high specificity. For example, LNAs can bind to STMN2 pre-RNA and repress premature polyadenylation of STMN2 pre-mRNA, and increase, restore, and/or stabilize STMN2 levels (e.g., STMN2 mRNA or protein levels) and/or activity (e.g., biological activity, for example, STMN2 activity); LNAs can bind to KCNQ2 pre-RNA and reduce mis-splicing of KCNQ2 pre-mRNA, and increase, restore, and/or stabilize KCNQ2 levels (e.g., KCNQ2 mRNA or protein levels) and/or activity (e.g., biological activity, for example, KCNQ2 activity); LNAs can bind to UNC13A pre-mRNA and prevent mis-splicing of UNC13A pre-mRNA, and increase, restore, and/or stabilize UNC13A levels (e.g., UNC13A mRNA or protein levels) and/or activity (e.g., biological activity, for example, UNC13A activity); and/or LNAs can bind to SMN2 pre-RNA and reduce mis-splicing of SMN2 pre-mRNA, and increase, restore, and/or stabilize SMN2 levels (e.g., SMN2 mRNA or protein levels) and/or activity (e.g., biological activity, for example, SMN2 activity).

Morpholino oligomers are oligonucleotide compounds that include bases attached to a backbone of methylenemorpholine rings linked through phosphorodiamidate groups. In certain embodiments, morpholino oligomers of the present invention can be designed to bind to specific pre-mRNA sequence of interest.

STMN2 Oligonucleotides Complementary to STMN2 Transcript with a Cryptic Exon

In some embodiments, a STMN2 AON includes a sequence that is at least 85% complementary to a sequence that shares at least 90% (e.g., 90%, 91%, 92%, 93%, 94%, 95%, 96%, 97%, 98%, 99%, or 100%) identity to a region of a STMN2 transcript that includes a cryptic exon (e.g., SEQ ID NO: 1339 or SEQ ID NO: 1341). In some embodiments, a STMN2 AON includes a sequence that is between 85 and 98% complementary to a sequence that shares at least 90% (e.g., 90%, 91%, 92%, 93%, 94%, 95%, 96%, 97%, 98%, 99%, or 100%) identity to a region of a STMN2 transcript that includes a cryptic exon (e.g., SEQ ID NO: 1339 or SEQ ID NO: 1341). In some embodiments, a STMN2 AON includes a sequence that is between 90-95% complementary to a sequence that shares at least 90% (e.g., 90%, 91%, 92%, 93%, 94%, 95%, 96%, 97%, 98%, 99%, or 100%) identity to a region of a STMN2 transcript that includes a cryptic exon (e.g., SEQ ID NO: 1339 or SEQ ID NO: 1341). In particular embodiments, a STMN2 AON includes a sequence that is at least 85% complementary to a sequence that shares at least 90% (e.g., 90%, 91%, 92%, 93%, 94%, 95%, 96%, 97%, 98%, 99%, or 100%) identity to a region of a STMN2 transcript that includes a cryptic exon (e.g., SEQ ID NO: 1339 or SEQ ID NO: 1341). In particular embodiments, a STMN2 AON includes a sequence that is between 84% to 88% complementary to a sequence that shares at least 90% (e.g., 90%, 91%, 92%, 93%, 94%, 95%, 96%, 97%, 98%, 99%, or 100%) identity to a region of a STMN2 transcript that includes a cryptic exon (e.g., SEQ ID NO: 1339 or SEQ ID NO: 1341). In particular embodiments, a STMN2 AON includes a sequence that is between 89% to 92% complementary to a sequence that shares at least 90% (e.g., 90%, 91%, 92%, 93%, 94%, 95%, 96%, 97%, 98%, 99%, or 100%) identity to a region of a STMN2 transcript that includes a cryptic exon (e.g., SEQ ID NO: 1339 or SEQ ID NO: 1341). In particular embodiments, a STMN2 AON includes a sequence that is between 94% to 96% complementary to a sequence that shares at least 90% (e.g., 90%, 91%, 92%, 93%, 94%, 95%, 96%, 97%, 98%, 99%, or 100%) identity to a region of a STMN2 transcript that includes a cryptic exon (e.g., SEQ ID NO: 1339 or SEQ ID NO: 1341).

In various embodiments, a STMN2 AON comprises a sequence that shares at least 85% identity with an equal length portion of any one of SEQ ID NOs: 1-466, SEQ ID NOs: 893-1338, SEQ ID NOs: 1342-1366, SEQ ID NOs: 1392-1664, or SEQ ID NOs: 10655-10669. In various embodiments, a STMN2 AON comprises a sequence that shares at least 90% identity with an equal length portion of any one of SEQ ID NOs: 1-466, SEQ ID NOs: 893-1338, SEQ ID NOs: 1342-1366, SEQ ID NOs: 1392-1664, or SEQ ID NOs: 10655-10669.

In various embodiments, the region of the STMN2 transcript targeted by the STMN2 AON is the cryptic exon sequence. In various embodiments, the region of the STMN2 transcript targeted by the STMN2 AON is a sequence located upstream or downstream (e.g., 100 or 200 bases upstream or downstream) of the cryptic exon sequence. In some embodiments, the STMN2 AON comprises a spacer and has a segment having at most 7 linked nucleosides. In some embodiments, the STMN2 AON comprises a spacer and has a segment having at most 6, 5, 4, 3, or 2 linked nucleosides.

STMN2 AON binding specificity can be assessed via measurement of parameters such as dissociation constant, melting temperature, or other criteria such as changes in protein or RNA expression levels or other assays that measure STMN2 activity or expression.

In some embodiments, a STMN2 AON can include a non-duplexed oligonucleotide. In some embodiments, a STMN2 AON can include a duplex of two oligonucleotides where the first oligonucleotide includes a nucleobase sequence that is completely or almost completely complementary to a STMN2 pre-mRNA sequence and the second oligonucleotide includes a nucleobase sequence that is complementary to the nucleobase sequence of the first oligonucleotide.

In some embodiments, a STMN2 AON can target STMN2 pre-mRNAs that include a cryptic exon produced from STMN2 genes of one or more species. For example, a STMN2 AON can target a STMN2 pre-mRNA, which includes a cryptic exon, of a mammalian STMN2 gene, for example, a human (i.e., Homo sapiens) STMN2 gene. In particular embodiments, the STMN2 AON targets a human STMN2 pre-mRNA, which includes a cryptic exon. In some embodiments, the STMN2 AON includes a nucleobase sequence that is complementary to a nucleobase sequence of a STMN2 gene or a STMN2 pre-mRNA, which includes a cryptic exon, or a portion thereof.

STMN2 AONs described herein include antisense oligonucleotides comprising the oligonucleotide sequences listed in Table 1 below:

TABLE 1
STMN2 AON Sequences, in each one or more spacers described in the present
disclosure are incorporated for generation of an
oligonucleotide of the present invention

| SEQ | | | SEQ | |
|---|---|---|---|---|
| ID | AON Sequence* | | ID | Target Sequence |
| NO: | (5′ → 3′) | Region | NO: | (5′ → 3′)) |
| 1 | GGAGGGATACCTGTATATTACAAGT | | 447 | ACTTGTAATATACAGGTATCCCTCC |
| 2 | AGGAGGGATACCTGTATATTACAAG | | 448 | CTTGTAATATACAGGTATCCCTCCT |
| 3 | CAGGAGGGATACCTGTATATTACAA | | 449 | TTGTAATATACAGGTATCCCTCCTG |
| 4 | CCAGGAGGGATACCTGTATATTACA | | 450 | TGTAATATACAGGTATCCCTCCTGG |
| 5 | ACCAGGAGGGATACCTGTATATTAC | | 451 | GTAATATACAGGTATCCCTCCTGGT |
| 6 | TACCAGGAGGGATACCTGTATATTA | | 452 | TAATATACAGGTATCCCTCCTGGTA |
| 7 | TTACCAGGAGGGATACCTGTATATT | | 453 | AATATACAGGTATCCCTCCTGGTAA |
| 8 | CTTACCAGGAGGGATACCTGTATAT | | 454 | ATATACAGGTATCCCTCCTGGTAAG |
| 9 | GCTTACCAGGAGGGATACCTGTATA | | 455 | TATACAGGTATCCCTCCTGGTAAGC |
| 10 | AGCTTACCAGGAGGGATACCTGTAT | | 456 | ATACAGGTATCCCTCCTGGTAAGCT |
| 11 | GAGCTTACCAGGAGGGATACCTGTA | | 457 | TACAGGTATCCCTCCTGGTAAGCTC |
| 12 | AGAGCTTACCAGGAGGGATACCTGT | | 458 | ACAGGTATCCCTCCTGGTAAGCTCT |
| 13 | CAGAGCTTACCAGGAGGGATACCTG | | 459 | CAGGTATCCCTCCTGGTAAGCTCTG |
| 14 | CCAGAGCTTACCAGGAGGGATACCT | | 460 | AGGTATCCCTCCTGGTAAGCTCTGG |
| 15 | ACCAGAGCTTACCAGGAGGGATACC | | 461 | GGTATCCCTCCTGGTAAGCTCTGGT |
| 16 | TACCAGAGCTTACCAGGAGGGATAC | | 462 | GTATCCCTCCTGGTAAGCTCTGGTA |
| 17 | ATACCAGAGCTTACCAGGAGGGATA | | 463 | TATCCCTCCTGGTAAGCTCTGGTAT |
| 18 | AATACCAGAGCTTACCAGGAGGGAT | | 464 | ATCCCTCCTGGTAAGCTCTGGTATT |
| 19 | TAATACCAGAGCTTACCAGGAGGGA | | 465 | TCCCTCCTGGTAAGCTCTGGTATTA |
| 20 | ATAATACCAGAGCTTACCAGGAGGG | | 466 | CCCTCCTGGTAAGCTCTGGTATTAT |
| 21 | CATAATACCAGAGCTTACCAGGAGG | | 467 | CCTCCTGGTAAGCTCTGGTATTATG |
| 22 | ACATAATACCAGAGCTTACCAGGAG | | 468 | CTCCTGGTAAGCTCTGGTATTATGT |
| 23 | GACATAATACCAGAGCTTACCAGGA | | 469 | TCCTGGTAAGCTCTGGTATTATGTC |
| 24 | AGACATAATACCAGAGCTTACCAGG | | 470 | CCTGGTAAGCTCTGGTATTATGTCT |
| 25 | AAGACATAATACCAGAGCTTACCAG | | 471 | CTGGTAAGCTCTGGTATTATGTCTT |
| 26 | TAAGACATAATACCAGAGCTTACCA | | 472 | TGGTAAGCTCTGGTATTATGTCTTA |
| 27 | TTAAGACATAATACCAGAGCTTACC | | 473 | GGTAAGCTCTGGTATTATGTCTTAA |
| 28 | GTTAAGACATAATACCAGAGCTTAC | | 474 | GTAAGCTCTGGTATTATGTCTTAAC |
| 29 | TGTTAAGACATAATACCAGAGCTTA | | 475 | TAAGCTCTGGTATTATGTCTTAACA |
| 30 | ATGTTAAGACATAATACCAGAGCTT | branch | 476 | AAGCTCTGGTATTATGTCTTAACAT |
| | | point 1 | | |
| 31 | AATGTTAAGACATAATACCAGAGCT | branch | 477 | AGCTCTGGTATTATGTCTTAACATT |
| | | point 1 | | |
| 32 | AAATGTTAAGACATAATACCAGAGC | branch | 478 | GCTCTGGTATTATGTCTTAACATTT |
| | | point 1 | | |
| 33 | AAAATGTTAAGACATAATACCAGAG | branch | 479 | CTCTGGTATTATGTCTTAACATTTT |
| | | point 1 | | |
| 34 | AAAAATGTTAAGACATAATACCAGA | branch | 480 | TCTGGTATTATGTCTTAACATTTTT |
| | | point 1 | | |
| 35 | TAAAAATGTTAAGACATAATACCAG | branch | 481 | CTGGTATTATGTCTTAACATTTTTA |
| | | point 1 | | |
| 36 | TTAAAAATGTTAAGACATAATACCA | branch | 482 | TGGTATTATGTCTTAACATTTTTAA |
| | | point 1 | | |
| 37 | TTTAAAAATGTTAAGACATAATACC | branch | 483 | GGTATTATGTCTTAACATTTTTAAA |
| | | point 1 | | |
| 38 | ATTTAAAAATGTTAAGACATAATAC | branch | 484 | GTATTATGTCTTAACATTTTTAAAT |
| | | point 1 | | |
| 39 | GATTTAAAAATGTTAAGACATAATA | branch | 485 | TATTATGTCTTAACATTTTTAAATC |
| | | point 1 | | |
| 40 | AGATTTAAAAATGTTAAGACATAAT | branch | 486 | ATTATGTCTTAACATTTTTAAATCT |
| | | point 1 | | |
| 41 | TAGATTTAAAAATGTTAAGACATAA | branch | 487 | TTATGTCTTAACATTTTTAAATCTA |
| | | point 1 | | |
| 42 | ATAGATTTAAAAATGTTAAGACATA | branch | 488 | TATGTCTTAACATTTTTAAATCTAT |
| | | point 1 | | |
| 43 | CATAGATTTAAAAATGTTAAGACAT | branch | 489 | ATGTCTTAACATTTTTAAATCTATG |
| | | point 1 | | |
| 44 | CCATAGATTTAAAAATGTTAAGACA | branch | 490 | TGTCTTAACATTTTTAAATCTATGG |
| | | point 1 | | |
| 45 | ACCATAGATTTAAAAATGTTAAGAC | branch | 491 | GTCTTAACATTTTTAAATCTATGGT |
| | | point 1 | | |
| 46 | TACCATAGATTTAAAAATGTTAAGA | branch | 492 | TCTTAACATTTTTAAATCTATGGTA |
| | | point 1 | | |
| 47 | TTACCATAGATTTAAAAATGTTAAG | | 493 | CTTAACATTTTTAAATCTATGGTAA |
| 48 | ATTACCATAGATTTAAAAATGTTAA | | 494 | TTAACATTTTTAAATCTATGGTAAT |
| 49 | GATTACCATAGATTTAAAAATGTTA | | 495 | TAACATTTTTAAATCTATGGTAATC |
| 50 | AGATTACCATAGATTTAAAAATGTT | Branch | 496 | AACATTTTTAAATCTATGGTAATCT |
| | | point 2 | | |
| 51 | AAGATTACCATAGATTTAAAAATGT | Branch | 497 | ACATTTTTAAATCTATGGTAATCTT |
| | | point 2 | | |
| 52 | AAAGATTACCATAGATTTAAAAATG | Branch | 498 | CATTTTTAAATCTATGGTAATCTTT |
| | | point 2 | | |
| 53 | TAAAGATTACCATAGATTTAAAAAT | Branch | 499 | ATTTTTAAATCTATGGTAATCTTTA |
| | | point 2 | | |
| 54 | GTAAAGATTACCATAGATTTAAAAA | Branch | 500 | TTTTTAAATCTATGGTAATCTTTAC |
| | | point 2 | | |
| 55 | TGTAAAGATTACCATAGATTTAAAA | Branch | 501 | TTTTAAATCTATGGTAATCTTTACA |
| | | point 2 | | |
| 56 | TTGTAAAGATTACCATAGATTTAAA | Branch | 502 | TTTAAATCTATGGTAATCTTTACAA |
| | | point 2 | | |
| 57 | TTTGTAAAGATTACCATAGATTTAA | Branch | 503 | TTAAATCTATGGTAATCTTTACAAA |
| | | point 2 | | |
| 58 | TTTTGTAAAGATTACCATAGATTTA | Branch | 504 | TAAATCTATGGTAATCTTTACAAAA |
| | | point 2 | | |
| 59 | ATTTTGTAAAGATTACCATAGATTT | Branch | 505 | AAATCTATGGTAATCTTTACAAAAT |
| | | point 2 | | |
| 60 | TATTTTGTAAAGATTACCATAGATT | Branch | 506 | AATCTATGGTAATCTTTACAAAATA |
| | | point 2 | | |
| 61 | ATATTTTGTAAAGATTACCATAGAT | Branch | 507 | ATCTATGGTAATCTTTACAAAATAT |
| | | point 2 | | |
| 62 | AATATTTTGTAAAGATTACCATAGA | Branch | 508 | TCTATGGTAATCTTTACAAAATATT |
| | | point 2 | | |
| 63 | AAATATTTTGTAAAGATTACCATAG | Branch | 509 | CTATGGTAATCTTTACAAAATATTT |
| | | point 2 | | |
| 64 | AAAATATTTTGTAAAGATTACCATA | Branch | 510 | TATGGTAATCTTTACAAAATATTTT |
| | | point 2 | | |
| 65 | TAAAATATTTTGTAAAGATTACCAT | Branch | 511 | ATGGTAATCTTTACAAAATATTTTA |
| | | point 2 | | |
| 66 | GTAAAATATTTTGTAAAGATTACCA | Branch | 512 | TGGTAATCTTTACAAAATATTTTAC |
| | | point 2 | | |
| 67 | AGTAAAATATTTTGTAAAGATTACC | | 513 | GGTAATCTTTACAAAATATTTTACT |
| 68 | AAGTAAAATATTTTGTAAAGATTAC | | 514 | GTAATCTTTACAAAATATTTTACTT |
| 69 | GAAGTAAAATATTTTGTAAAGATTA | | 515 | TAATCTTTACAAAATATTTTACTTC |
| 70 | GGAAGTAAAATATTTTGTAAAGATT | | 516 | AATCTTTACAAAATATTTTACTTCC |
| 71 | CGGAAGTAAAATATTTTGTAAAGAT | | 517 | ATCTTTACAAAATATTTTACTTCCG |
| 72 | TCGGAAGTAAAATATTTTGTAAAGA | | 518 | TCTTTACAAAATATTTTACTTCCGA |
| 73 | TTCGGAAGTAAAATATTTTGTAAAG | | 519 | CTTTACAAAATATTTTACTTCCGAA |
| 74 | GTTCGGAAGTAAAATATTTTGTAAA | | 520 | TTTACAAAATATTTTACTTCCGAAC |
| 75 | AGTTCGGAAGTAAAATATTTTGTAA | | 521 | TTACAAAATATTTTACTTCCGAACT |
| 76 | GAGTTCGGAAGTAAAATATTTTGTA | | 522 | TACAAAATATTTTACTTCCGAACTC |
| 77 | TGAGTTCGGAAGTAAAATATTTTGT | | 523 | ACAAAATATTTTACTTCCGAACTCA |
| 78 | ATGAGTTCGGAAGTAAAATATTTTG | | 524 | CAAAATATTTTACTTCCGAACTCAT |
| 79 | TATGAGTTCGGAAGTAAAATATTTT | | 525 | AAAATATTTTACTTCCGAACTCATA |
| 80 | ATATGAGTTCGGAAGTAAAATATTT | | 526 | AAATATTTTACTTCCGAACTCATAT |
| 81 | TATATGAGTTCGGAAGTAAAATATT | | 527 | AATATTTTACTTCCGAACTCATATA |
| 82 | GTATATGAGTTCGGAAGTAAAATAT | | 528 | ATATTTTACTTCCGAACTCATATAC |
| 83 | GGTATATGAGTTCGGAAGTAAAATA | | 529 | TATTTTACTTCCGAACTCATATACC |
| 84 | AGGTATATGAGTTCGGAAGTAAAAT | | 530 | ATTTTACTTCCGAACTCATATACCT |
| 85 | CAGGTATATGAGTTCGGAAGTAAAA | | 531 | TTTTACTTCCGAACTCATATACCTG |
| 86 | CCAGGTATATGAGTTCGGAAGTAAA | | 532 | TTTACTTCCGAACTCATATACCTGG |
| 87 | CCCAGGTATATGAGTTCGGAAGTAA | | 533 | TTACTTCCGAACTCATATACCTGGG |
| 88 | CCCCAGGTATATGAGTTCGGAAGTA | | 534 | TACTTCCGAACTCATATACCTGGGG |
| 89 | TCCCCAGGTATATGAGTTCGGAAGT | | 535 | ACTTCCGAACTCATATACCTGGGGA |
| 90 | ATCCCCAGGTATATGAGTTCGGAAG | | 536 | CTTCCGAACTCATATACCTGGGGAT |
| 91 | AATCCCCAGGTATATGAGTTCGGAA | | 537 | TTCCGAACTCATATACCTGGGGATT |
| 92 | AAATCCCCAGGTATATGAGTTCGGA | | 538 | TCCGAACTCATATACCTGGGGATTT |
| 93 | AAAATCCCCAGGTATATGAGTTCGG | | 539 | CCGAACTCATATACCTGGGGATTTT |
| 94 | TAAAATCCCCAGGTATATGAGTTCG | | 540 | CGAACTCATATACCTGGGGATTTTA |
| 95 | ATAAAATCCCCAGGTATATGAGTTC | | 541 | GAACTCATATACCTGGGGATTTTAT |
| 96 | AATAAAATCCCCAGGTATATGAGTT | | 542 | AACTCATATACCTGGGGATTTTATT |
| 97 | TAATAAAATCCCCAGGTATATGAGT | | 543 | ACTCATATACCTGGGGATTTTATTA |
| 98 | GTAATAAAATCCCCAGGTATATGAG | | 544 | CTCATATACCTGGGGATTTTATTAC |
| 99 | AGTAATAAAATCCCCAGGTATATGA | | 545 | TCATATACCTGGGGATTTTATTACT |
| 100 | GAGTAATAAAATCCCCAGGTATATG | | 546 | CATATACCTGGGGATTTTATTACTC |
| 101 | AGAGTAATAAAATCCCCAGGTATAT | | 547 | ATATACCTGGGGATTTTATTACTCT |
| 102 | CAGAGTAATAAAATCCCCAGGTATA | | 548 | TATACCTGGGGATTTTATTACTCTG |
| 103 | CCAGAGTAATAAAATCCCCAGGTAT | | 549 | ATACCTGGGGATTTTATTACTCTGG |
| 104 | CCCAGAGTAATAAAATCCCCAGGTA | | 550 | TACCTGGGGATTTTATTACTCTGGG |
| 105 | TCCCAGAGTAATAAAATCCCCAGGT | | 551 | ACCTGGGGATTTTATTACTCTGGGA |
| 106 | TTCCCAGAGTAATAAAATCCCCAGG | | 552 | CCTGGGGATTTTATTACTCTGGGAA |
| 107 | ATTCCCAGAGTAATAAAATCCCCAG | | 553 | CTGGGGATTTTATTACTCTGGGAAT |
| 108 | AATTCCCAGAGTAATAAAATCCCCA | | 554 | TGGGGATTTTATTACTCTGGGAATT |
| 109 | TAATTCCCAGAGTAATAAAATCCCC | | 555 | GGGGATTTTATTACTCTGGGAATTA |
| 110 | ATAATTCCCAGAGTAATAAAATCCC | | 556 | GGGATTTTATTACTCTGGGAATTAT |
| 111 | CATAATTCCCAGAGTAATAAAATCC | | 557 | GGATTTTATTACTCTGGGAATTATG |
| 112 | ACATAATTCCCAGAGTAATAAAATC | | 558 | GATTTTATTACTCTGGGAATTATGT |
| 113 | CACATAATTCCCAGAGTAATAAAAT | | 559 | ATTTTATTACTCTGGGAATTATGTG |
| 114 | ACACATAATTCCCAGAGTAATAAAA | | 560 | TTTTATTACTCTGGGAATTATGTGT |
| 115 | AACACATAATTCCCAGAGTAATAAA | | 561 | TTTATTACTCTGGGAATTATGTGTT |
| 116 | GAACACATAATTCCCAGAGTAATAA | | 562 | TTATTACTCTGGGAATTATGTGTTC |
| 117 | AGAACACATAATTCCCAGAGTAATA | | 563 | TATTACTCTGGGAATTATGTGTTCT |
| 118 | CAGAACACATAATTCCCAGAGTAAT | | 564 | ATTACTCTGGGAATTATGTGTTCTG |
| 119 | GCAGAACACATAATTCCCAGAGTAA | | 565 | TTACTCTGGGAATTATGTGTTCTGC |
| 120 | GGCAGAACACATAATTCCCAGAGTA | | 566 | TACTCTGGGAATTATGTGTTCTGCC |
| 121 | GGGCAGAACACATAATTCCCAGAGT | | 567 | ACTCTGGGAATTATGTGTTCTGCCC |
| 122 | GGGGCAGAACACATAATTCCCAGAG | | 568 | CTCTGGGAATTATGTGTTCTGCCCC |
| 123 | TGGGGCAGAACACATAATTCCCAGA | | 569 | TCTGGGAATTATGTGTTCTGCCCCA |
| 124 | ATGGGGCAGAACACATAATTCCCAG | | 570 | CTGGGAATTATGTGTTCTGCCCCAT |
| 125 | GATGGGGCAGAACACATAATTCCCA | | 571 | TGGGAATTATGTGTTCTGCCCCATC |
| 126 | TGATGGGGCAGAACACATAATTCCC | | 572 | GGGAATTATGTGTTCTGCCCCATCA |
| 127 | GTGATGGGGCAGAACACATAATTCC | | 573 | GGAATTATGTGTTCTGCCCCATCAC |
| 128 | AGTGATGGGGCAGAACACATAATTC | | 574 | GAATTATGTGTTCTGCCCCATCACT |
| 129 | GAGTGATGGGGCAGAACACATAATT | Branch | 575 | AATTATGTGTTCTGCCCCATCACTC |
| | | point 3 | | |
| 130 | AGAGTGATGGGGCAGAACACATAAT | Branch | 576 | ATTATGTGTTCTGCCCCATCACTCT |
| | | point 3 | | |
| 131 | GAGAGTGATGGGGCAGAACACATAA | Branch | 577 | TTATGTGTTCTGCCCCATCACTCTC |
| | | point 3 | | |
| 132 | AGAGAGTGATGGGGCAGAACACATA | Branch | 578 | TATGTGTTCTGCCCCATCACTCTCT |
| | | point 3 | | |
| 133 | GAGAGAGTGATGGGGCAGAACACAT | Branch | 579 | ATGTGTTCTGCCCCATCACTCTCTC |
| | | point 3 | | |
| 134 | AGAGAGAGTGATGGGGCAGAACACA | Branch | 580 | TGTGTTCTGCCCCATCACTCTCTCT |
| | | point 3 | | |
| 135 | AAGAGAGAGTGATGGGGCAGAACAC | Branch | 581 | GTGTTCTGCCCCATCACTCTCTCTT |
| | | point 3 | | |
| 136 | TAAGAGAGAGTGATGGGGCAGAACA | Branch | 582 | TGTTCTGCCCCATCACTCTCTCTTA |
| | | point 3 | | |
| 137 | TTAAGAGAGAGTGATGGGGCAGAAC | Branch | 583 | GTTCTGCCCCATCACTCTCTCTTAA |
| | | point 3 | | |
| 138 | ATTAAGAGAGAGTGATGGGGCAGAA | Branch | 584 | TTCTGCCCCATCACTCTCTCTTAAT |
| | | point 3 | | |
| 139 | AATTAAGAGAGAGTGATGGGGCAGA | Branch | 585 | TCTGCCCCATCACTCTCTCTTAATT |
| | | point 3 | | |
| 140 | CAATTAAGAGAGAGTGATGGGGCAG | Branch | 586 | CTGCCCCATCACTCTCTCTTAATTG |
| | | point 3 | | |
| 141 | CCAATTAAGAGAGAGTGATGGGGCA | Branch | 587 | TGCCCCATCACTCTCTCTTAATTGG |
| | | point 3 | | |
| 142 | TCCAATTAAGAGAGAGTGATGGGGC | Branch | 588 | GCCCCATCACTCTCTCTTAATTGGA |
| | | point 3 | | |
| 143 | ATCCAATTAAGAGAGAGTGATGGGG | Branch | 589 | CCCCATCACTCTCTCTTAATTGGAT |
| | | point 3 | | |
| 144 | AATCCAATTAAGAGAGAGTGATGGG | Branch | 590 | CCCATCACTCTCTCTTAATTGGATT |
| | | point 3 | | |
| 145 | AAATCCAATTAAGAGAGAGTGATGG | Branch | 591 | CCATCACTCTCTCTTAATTGGATTT |
| | | point 3 | | |
| 146 | AAAATCCAATTAAGAGAGAGTGATG | | 592 | CATCACTCTCTCTTAATTGGATTTT |
| 147 | AAAAATCCAATTAAGAGAGAGTGAT | | 593 | ATCACTCTCTCTTAATTGGATTTTT |
| 148 | TAAAAATCCAATTAAGAGAGAGTGA | | 594 | TCACTCTCTCTTAATTGGATTTTTA |
| 149 | TTAAAAATCCAATTAAGAGAGAGTG | | 595 | CACTCTCTCTTAATTGGATTTTTAA |
| 150 | TTTAAAAATCCAATTAAGAGAGAGT | | 596 | ACTCTCTCTTAATTGGATTTTTAAA |
| 151 | TTTTAAAAATCCAATTAAGAGAGAG | | 597 | CTCTCTCTTAATTGGATTTTTAAAA |
| 152 | ATTTTAAAAATCCAATTAAGAGAGA | | 598 | TCTCTCTTAATTGGATTTTTAAAAT |
| 153 | AATTTTAAAAATCCAATTAAGAGAG | | 599 | CTCTCTTAATTGGATTTTTAAAATT |
| 154 | TAATTTTAAAAATCCAATTAAGAGA | | 600 | TCTCTTAATTGGATTTTTAAAATTA |
| 155 | ATAATTTTAAAAATCCAATTAAGAG | | 601 | CTCTTAATTGGATTTTTAAAATTAT |
| 156 | TATAATTTTAAAAATCCAATTAAGA | | 602 | TCTTAATTGGATTTTTAAAATTATA |
| 157 | ATATAATTTTAAAAATCCAATTAAG | | 603 | CTTAATTGGATTTTTAAAATTATAT |
| 158 | AATATAATTTTAAAAATCCAATTAA | | 604 | TTAATTGGATTTTTAAAATTATATT |
| 159 | GAATATAATTTTAAAAATCCAATTA | | 605 | TAATTGGATTTTTAAAATTATATTC |
| 160 | TGAATATAATTTTAAAAATCCAATT | | 606 | AATTGGATTTTTAAAATTATATTCA |
| 161 | ATGAATATAATTTTAAAAATCCAAT | | 607 | ATTGGATTTTTAAAATTATATTCAT |
| 162 | TATGAATATAATTTTAAAAATCCAA | | 608 | TTGGATTTTTAAAATTATATTCATA |
| 163 | ATATGAATATAATTTTAAAAATCCA | | 609 | TGGATTTTTAAAATTATATTCATAT |
| 164 | AATATGAATATAATTTTAAAAATCC | | 610 | GGATTTTTAAAATTATATTCATATT |
| 165 | CAATATGAATATAATTTTAAAAATC | | 611 | GATTTTTAAAATTATATTCATATTG |
| 166 | GCAATATGAATATAATTTTAAAAAT | | 612 | ATTTTTAAAATTATATTCATATTGC |
| 167 | TGCAATATGAATATAATTTTAAAAA | | 613 | TTTTTAAAATTATATTCATATTGCA |
| 168 | CTGCAATATGAATATAATTTTAAAA | | 614 | TTTTAAAATTATATTCATATTGCAG |
| 169 | CCTGCAATATGAATATAATTTTAAA | | 615 | TTTAAAATTATATTCATATTGCAGG |
| 170 | TCCTGCAATATGAATATAATTTTAA | | 616 | TTAAAATTATATTCATATTGCAGGA |
| 171 | GTCCTGCAATATGAATATAATTTTA | Acceptor | 617 | TAAAATTATATTCATATTGCAGGAC |
| | | site | | |
| 172 | AGTCCTGCAATATGAATATAATTTT | Acceptor | 618 | AAAATTATATTCATATTGCAGGACT |
| | | site | | |
| 173 | GAGTCCTGCAATATGAATATAATTT | Acceptor | 619 | AAATTATATTCATATTGCAGGACTC |
| | | site | | |
| 174 | CGAGTCCTGCAATATGAATATAATT | Acceptor | 620 | AATTATATTCATATTGCAGGACTCG |
| | | site | | |
| 175 | CCGAGTCCTGCAATATGAATATAAT | Acceptor | 621 | ATTATATTCATATTGCAGGACTCGG |
| | | site | | |
| 176 | GCCGAGTCCTGCAATATGAATATAA | Acceptor | 622 | TTATATTCATATTGCAGGACTCGGC |
| | | site | | |
| 177 | TGCCGAGTCCTGCAATATGAATATA | Acceptor | 623 | TATATTCATATTGCAGGACTCGGCA |
| | | site | | |
| 178 | CTGCCGAGTCCTGCAATATGAATAT | Acceptor | 624 | ATATTCATATTGCAGGACTCGGCAG |
| | | site | | |
| 179 | TCTGCCGAGTCCTGCAATATGAATA | Acceptor | 625 | TATTCATATTGCAGGACTCGGCAGA |
| | | site | | |
| 180 | TTCTGCCGAGTCCTGCAATATGAAT | Acceptor | 626 | ATTCATATTGCAGGACTCGGCAGAA |
| | | site | | |
| 181 | CTTCTGCCGAGTCCTGCAATATGAA | Acceptor | 627 | TTCATATTGCAGGACTCGGCAGAAG |
| | | site | | |
| 182 | TCTTCTGCCGAGTCCTGCAATATGA | Acceptor | 628 | TCATATTGCAGGACTCGGCAGAAGA |
| | | site | | |
| 183 | GTCTTCTGCCGAGTCCTGCAATATG | Acceptor | 629 | CATATTGCAGGACTCGGCAGAAGAC |
| | | site | | |
| 184 | GGTCTTCTGCCGAGTCCTGCAATAT | Acceptor | 630 | ATATTGCAGGACTCGGCAGAAGACC |
| | | site | | |
| 185 | AGGTCTTCTGCCGAGTCCTGCAATA | Acceptor | 631 | TATTGCAGGACTCGGCAGAAGACCT |
| | | site | | |
| 186 | AAGGTCTTCTGCCGAGTCCTGCAAT | Acceptor | 632 | ATTGCAGGACTCGGCAGAAGACCTT |
| | | site | | |
| 187 | GAAGGTCTTCTGCCGAGTCCTGCAA | Acceptor | 633 | TTGCAGGACTCGGCAGAAGACCTTC |
| | | site | | |
| 188 | CGAAGGTCTTCTGCCGAGTCCTGCA | Acceptor | 634 | TGCAGGACTCGGCAGAAGACCTTCG |
| | | site | | |
| 189 | TCGAAGGTCTTCTGCCGAGTCCTGC | Acceptor | 635 | GCAGGACTCGGCAGAAGACCTTCGA |
| | | site | | |
| 190 | CTCGAAGGTCTTCTGCCGAGTCCTG | Acceptor | 636 | CAGGACTCGGCAGAAGACCTTCGAG |
| | | site | | |
| 191 | TCTCGAAGGTCTTCTGCCGAGTCCT | ESE | 637 | AGGACTCGGCAGAAGACCTTCGAGA |
| | | Binding | | |
| 192 | CTCTCGAAGGTCTTCTGCCGAGTCC | ESE | 638 | GGACTCGGCAGAAGACCTTCGAGAG |
| | | Binding | | |
| 193 | TCTCTCGAAGGTCTTCTGCCGAGTC | ESE | 639 | GACTCGGCAGAAGACCTTCGAGAGA |
| | | Binding | | |
| 194 | TTCTCTCGAAGGTCTTCTGCCGAGT | ESE | 640 | ACTCGGCAGAAGACCTTCGAGAGAA |
| | | Binding | | |
| 195 | TTTCTCTCGAAGGTCTTCTGCCGAG | ESE | 641 | CTCGGCAGAAGACCTTCGAGAGAAA |
| | | Binding | | |
| 196 | CTTTCTCTCGAAGGTCTTCTGCCGA | ESE | 642 | TCGGCAGAAGACCTTCGAGAGAAAG |
| | | Binding | | |
| 197 | CCTTTCTCTCGAAGGTCTTCTGCCG | ESE | 643 | CGGCAGAAGACCTTCGAGAGAAAGG |
| | | Binding | | |
| 198 | ACCTTTCTCTCGAAGGTCTTCTGCC | ESE | 644 | GGCAGAAGACCTTCGAGAGAAAGGT |
| | | Binding | | |
| 199 | TACCTTTCTCTCGAAGGTCTTCTGC | ESE | 645 | GCAGAAGACCTTCGAGAGAAAGGTA |
| | | Binding | | |
| 200 | CTACCTTTCTCTCGAAGGTCTTCTG | ESE | 646 | CAGAAGACCTTCGAGAGAAAGGTAG |
| | | Binding | | |
| 201 | TCTACCTTTCTCTCGAAGGTCTTCT | ESE | 647 | AGAAGACCTTCGAGAGAAAGGTAGA |
| | | Binding | | |
| 202 | TTCTACCTTTCTCTCGAAGGTCTTC | ESE | 648 | GAAGACCTTCGAGAGAAAGGTAGAA |
| | | Binding | | |
| 203 | TTTCTACCTTTCTCTCGAAGGTCTT | ESE | 649 | AAGACCTTCGAGAGAAAGGTAGAAA |
| | | Binding | | |
| 204 | TTTTCTACCTTTCTCTCGAAGGTCT | ESE | 650 | AGACCTTCGAGAGAAAGGTAGAAAA |
| | | Binding | | |
| 205 | ATTTTCTACCTTTCTCTCGAAGGTC | ESE | 651 | GACCTTCGAGAGAAAGGTAGAAAAT |
| | | Binding | | |
| 206 | TATTTTCTACCTTTCTCTCGAAGGT | ESE | 652 | ACCTTCGAGAGAAAGGTAGAAAATA |
| | | Binding | | |
| 207 | TTATTTTCTACCTTTCTCTCGAAGG | ESE | 653 | CCTTCGAGAGAAAGGTAGAAAATAA |
| | | Binding | | |
| 208 | CTTATTTTCTACCTTTCTCTCGAAG | ESE | 654 | CTTCGAGAGAAAGGTAGAAAATAAG |
| | | Binding | | |
| 209 | TCTTATTTTCTACCTTTCTCTCGAA | ESE | 655 | TTCGAGAGAAAGGTAGAAAATAAGA |
| | | Binding | | |
| 210 | TTCTTATTTTCTACCTTTCTCTCGA | ESE | 656 | TCGAGAGAAAGGTAGAAAATAAGAA |
| | | Binding | | |
| 211 | ATTCTTATTTTCTACCTTTCTCTCG | ESE | 657 | CGAGAGAAAGGTAGAAAATAAGAAT |
| | | Binding | | |
| 212 | AATTCTTATTTTCTACCTTTCTCTC | ESE | 658 | GAGAGAAAGGTAGAAAATAAGAATT |
| | | Binding | | |
| 213 | AAATTCTTATTTTCTACCTTTCTCT | ESE | 659 | AGAGAAAGGTAGAAAATAAGAATTT |
| | | Binding | | |
| 214 | CAAATTCTTATTTTCTACCTTTCTC | ESE | 660 | GAGAAAGGTAGAAAATAAGAATTTG |
| | | Binding | | |
| 215 | CCAAATTCTTATTTTCTACCTTTCT | ESE | 661 | AGAAAGGTAGAAAATAAGAATTTGG |
| | | Binding | | |
| 216 | GCCAAATTCTTATTTTCTACCTTTC | ESE | 662 | GAAAGGTAGAAAATAAGAATTTGGC |
| | | Binding | | |
| 217 | AGCCAAATTCTTATTTTCTACCTTT | ESE | 663 | AAAGGTAGAAAATAAGAATTTGGCT |
| | | Binding | | |
| 218 | GAGCCAAATTCTTATTTTCTACCTT | ESE | 664 | AAGGTAGAAAATAAGAATTTGGCTC |
| | | Binding | | |
| 219 | AGAGCCAAATTCTTATTTTCTACCT | ESE | 665 | AGGTAGAAAATAAGAATTTGGCTCT |
| | | Binding | | |
| 220 | GAGAGCCAAATTCTTATTTTCTACC | ESE | 666 | GGTAGAAAATAAGAATTTGGCTCTC |
| | | Binding | | |
| 221 | AGAGAGCCAAATTCTTATTTTCTAC | ESE | 667 | GTAGAAAATAAGAATTTGGCTCTCT |
| | | Binding | | |
| 222 | CAGAGAGCCAAATTCTTATTTTCTA | | 668 | TAGAAAATAAGAATTTGGCTCTCTG |
| 223 | ACAGAGAGCCAAATTCTTATTTTCT | | 669 | AGAAAATAAGAATTTGGCTCTCTGT |
| 224 | CACAGAGAGCCAAATTCTTATTTTC | | 670 | GAAAATAAGAATTTGGCTCTCTGTG |
| 225 | ACACAGAGAGCCAAATTCTTATTTT | | 671 | AAAATAAGAATTTGGCTCTCTGTGT |
| 226 | CACACAGAGAGCCAAATTCTTATTT | Overlaps | 672 | AAATAAGAATTTGGCTCTCTGTGTG |
| | | TDP-43 | | |
| | | site 1 | | |
| 227 | TCACACAGAGAGCCAAATTCTTATT | Overlaps | 673 | AATAAGAATTTGGCTCTCTGTGTGA |
| | | TDP-43 | | |
| | | site 1 | | |
| 228 | CTCACACAGAGAGCCAAATTCTTAT | Overlaps | 674 | ATAAGAATTTGGCTCTCTGTGTGAG |
| | | TDP-43 | | |
| | | site 1 | | |
| 229 | GCTCACACAGAGAGCCAAATTCTTA | Overlaps | 675 | TAAGAATTTGGCTCTCTGTGTGAGC |
| | | TDP-43 | | |
| | | site 1 | | |
| 230 | TGCTCACACAGAGAGCCAAATTCTT | Overlaps | 676 | AAGAATTTGGCTCTCTGTGTGAGCA |
| | | TDP-43 | | |
| | | site 1 | | |
| 231 | ATGCTCACACAGAGAGCCAAATTCT | Overlaps | 677 | AGAATTTGGCTCTCTGTGTGAGCAT |
| | | TDP-43 | | |
| | | site 1 | | |
| 232 | CATGCTCACACAGAGAGCCAAATTC | Overlaps | 678 | GAATTTGGCTCTCTGTGTGAGCATG |
| | | TDP-43 | | |
| | | site 1 | | |
| 233 | ACATGCTCACACAGAGAGCCAAATT | Overlaps | 679 | AATTTGGCTCTCTGTGTGAGCATGT |
| | | TDP-43 | | |
| | | site 1 | | |
| 234 | CACATGCTCACACAGAGAGCCAAAT | Overlaps | 680 | ATTTGGCTCTCTGTGTGAGCATGTG |
| | | TDP-43 | | |
| | | site 1 | | |
| 235 | ACACATGCTCACACAGAGAGCCAAA | Overlaps | 681 | TTTGGCTCTCTGTGTGAGCATGTGT |
| | | TDP-43 | | |
| | | site 1 | | |
| 236 | CACACATGCTCACACAGAGAGCCAA | Overlaps | 682 | TTGGCTCTCTGTGTGAGCATGTGTG |
| | | TDP-43 | | |
| | | site 1 & | | |
| | | 2 | | |
| 237 | GCACACATGCTCACACAGAGAGCCA | Overlaps | 683 | TGGCTCTCTGTGTGAGCATGTGTGC |
| | | TDP-43 | | |
| | | site 1 & | | |
| | | 2 | | |
| 238 | CGCACACATGCTCACACAGAGAGCC | Overlaps | 684 | GGCTCTCTGTGTGAGCATGTGTGCG |
| | | TDP-43 | | |
| | | site 1 & | | |
| | | 2 | | |
| 239 | ACGCACACATGCTCACACAGAGAGC | Overlaps | 685 | GCTCTCTGTGTGAGCATGTGTGCGT |
| | | TDP-43 | | |
| | | site 1 & | | |
| | | 2 | | |
| 240 | CACGCACACATGCTCACACAGAGAG | Overlaps | 686 | CTCTCTGTGTGAGCATGTGTGCGTG |
| | | TDP-43 | | |
| | | site 1 & | | |
| | | 2 | | |
| 241 | ACACGCACACATGCTCACACAGAGA | Overlaps | 687 | TCTCTGTGTGAGCATGTGTGCGTGT |
| | | TDP-43 | | |
| | | site 1 & | | |
| | | 2 | | |
| 242 | CACACGCACACATGCTCACACAGAG | Overlaps | 688 | CTCTGTGTGAGCATGTGTGCGTGTG |
| | | TDP-43 | | |
| | | site 1 & | | |
| | | 2 | | |
| 243 | ACACACGCACACATGCTCACACAGA | Overlaps | 689 | TCTGTGTGAGCATGTGTGCGTGTGT |
| | | TDP-43 | | |
| | | site 1 & | | |
| | | 2 | | |
| 244 | CACACACGCACACATGCTCACACAG | Overlaps | 690 | CTGTGTGAGCATGTGTGCGTGTGTG |
| | | TDP-43 | | |
| | | site 1 & | | |
| | | 2 & 3 | | |
| 245 | GCACACACGCACACATGCTCACACA | Overlaps | 691 | TGTGTGAGCATGTGTGCGTGTGTGC |
| | | TDP-43 | | |
| | | site 1 & | | |
| | | 2 & 3 | | |
| 246 | CGCACACACGCACACATGCTCACAC | Overlaps | 692 | GTGTGAGCATGTGTGCGTGTGTGCG |
| | | TDP-43 | | |
| | | site 2 & | | |
| | | 3 | | |
| 247 | TCGCACACACGCACACATGCTCACA | Overlaps | 693 | TGTGAGCATGTGTGCGTGTGTGCGA |
| | | TDP-43 | | |
| | | site 2 & | | |
| | | 3 | | |
| 248 | CTCGCACACACGCACACATGCTCAC | Overlaps | 694 | GTGAGCATGTGTGCGTGTGTGCGAG |
| | | TDP-43 | | |
| | | site 2 & | | |
| | | 3 | | |
| 249 | TCTCGCACACACGCACACATGCTCA | Overlaps | 695 | TGAGCATGTGTGCGTGTGTGCGAGA |
| | | TDP-43 | | |
| | | site 2 & | | |
| | | 3 | | |
| 250 | CTCTCGCACACACGCACACATGCTC | Overlaps | 696 | GAGCATGTGTGCGTGTGTGCGAGAG |
| | | TDP-43 | | |
| | | site 2 & | | |
| | | 3 | | |
| 251 | TCTCTCGCACACACGCACACATGCT | Overlaps | 697 | AGCATGTGTGCGTGTGTGCGAGAGA |
| | | TDP-43 | | |
| | | site 2 & | | |
| | | 3 | | |
| 252 | CTCTCTCGCACACACGCACACATGC | Overlaps | 698 | GCATGTGTGCGTGTGTGCGAGAGAG |
| | | TDP-43 | | |
| | | site 2 & | | |
| | | 3 | | |
| 253 | TCTCTCTCGCACACACGCACACATG | Overlaps | 699 | CATGTGTGCGTGTGTGCGAGAGAGA |
| | | TDP-43 | | |
| | | site 2 & | | |
| | | 3 | | |
| 254 | CTCTCTCTCGCACACACGCACACAT | Overlaps | 700 | ATGTGTGCGTGTGTGCGAGAGAGAG |
| | | TDP-43 | | |
| | | site 2 & | | |
| | | 3 | | |
| 255 | TCTCTCTCTCGCACACACGCACACA | Overlaps | 701 | TGTGTGCGTGTGTGCGAGAGAGAGA |
| | | TDP-43 | | |
| | | site 2 & | | |
| | | 3 | | |
| 256 | CTCTCTCTCTCGCACACACGCACAC | Overlaps | 702 | GTGTGCGTGTGTGCGAGAGAGAGAG |
| | | TDP-43 | | |
| | | site 3 | | |
| 257 | TCTCTCTCTCTCGCACACACGCACA | Overlaps | 703 | TGTGCGTGTGTGCGAGAGAGAGAGA |
| | | TDP-43 | | |
| | | site 3 | | |
| 258 | GTCTCTCTCTCTCGCACACACGCAC | Overlaps | 704 | GTGCGTGTGTGCGAGAGAGAGAGAC |
| | | TDP-43 | | |
| | | site 3 | | |
| 259 | TGTCTCTCTCTCTCGCACACACGCA | Overlaps | 705 | TGCGTGTGTGCGAGAGAGAGAGACA |
| | | TDP-43 | | |
| | | site 3 | | |
| 260 | CTGTCTCTCTCTCTCGCACACACGC | Overlaps | 706 | GCGTGTGTGCGAGAGAGAGAGACAG |
| | | TDP-43 | | |
| | | site 3 | | |
| 261 | TCTGTCTCTCTCTCTCGCACACACG | Overlaps | 707 | CGTGTGTGCGAGAGAGAGAGACAGA |
| | | TDP-43 | | |
| | | site 3 | | |
| 262 | GTCTGTCTCTCTCTCTCGCACACAC | Overlaps | 708 | GTGTGTGCGAGAGAGAGAGACAGAC |
| | | TDP-43 | | |
| | | site 3 | | |
| 263 | TGTCTGTCTCTCTCTCTCGCACACA | Overlaps | 709 | TGTGTGCGAGAGAGAGAGACAGACA |
| | | TDP-43 | | |
| | | site 3 | | |
| 264 | CTGTCTGTCTCTCTCTCTCGCACAC | | 710 | GTGTGCGAGAGAGAGAGACAGACAG |
| 265 | GCTGTCTGTCTCTCTCTCTCGCACA | | 711 | TGTGCGAGAGAGAGAGACAGACAGC |
| 266 | GGCTGTCTGTCTCTCTCTCTCGCAC | | 712 | GTGCGAGAGAGAGAGACAGACAGCC |
| 267 | AGGCTGTCTGTCTCTCTCTCTCGCA | | 713 | TGCGAGAGAGAGAGACAGACAGCCT |
| 268 | CAGGCTGTCTGTCTCTCTCTCTCGC | | 714 | GCGAGAGAGAGAGACAGACAGCCTG |
| 269 | GCAGGCTGTCTGTCTCTCTCTCTCG | | 715 | CGAGAGAGAGAGACAGACAGCCTGC |
| 270 | GGCAGGCTGTCTGTCTCTCTCTCTC | | 716 | GAGAGAGAGAGACAGACAGCCTGCC |
| 271 | AGGCAGGCTGTCTGTCTCTCTCTCT | | 717 | AGAGAGAGAGACAGACAGCCTGCCT |
| 272 | TAGGCAGGCTGTCTGTCTCTCTCTC | | 718 | GAGAGAGAGACAGACAGCCTGCCTA |
| 273 | TTAGGCAGGCTGTCTGTCTCTCTCT | | 719 | AGAGAGAGACAGACAGCCTGCCTAA |
| 274 | CTTAGGCAGGCTGTCTGTCTCTCTC | | 720 | GAGAGAGACAGACAGCCTGCCTAAG |
| 275 | TCTTAGGCAGGCTGTCTGTCTCTCT | | 721 | AGAGAGACAGACAGCCTGCCTAAGA |
| 276 | TTCTTAGGCAGGCTGTCTGTCTCTC | | 722 | GAGAGACAGACAGCCTGCCTAAGAA |
| 277 | CTTCTTAGGCAGGCTGTCTGTCTCT | | 723 | AGAGACAGACAGCCTGCCTAAGAAG |
| 278 | TCTTCTTAGGCAGGCTGTCTGTCTC | | 724 | GAGACAGACAGCCTGCCTAAGAAGA |
| 279 | TTCTTCTTAGGCAGGCTGTCTGTCT | | 725 | AGACAGACAGCCTGCCTAAGAAGAA |
| 280 | TTTCTTCTTAGGCAGGCTGTCTGTC | | 726 | GACAGACAGCCTGCCTAAGAAGAAA |
| 281 | ATTTCTTCTTAGGCAGGCTGTCTGT | | 727 | ACAGACAGCCTGCCTAAGAAGAAAT |
| 282 | CATTTCTTCTTAGGCAGGCTGTCTG | | 728 | CAGACAGCCTGCCTAAGAAGAAATG |
| 283 | TCATTTCTTCTTAGGCAGGCTGTCT | | 729 | AGACAGCCTGCCTAAGAAGAAATGA |
| 284 | TTCATTTCTTCTTAGGCAGGCTGTC | | 730 | GACAGCCTGCCTAAGAAGAAATGAA |
| 285 | ATTCATTTCTTCTTAGGCAGGCTGT | | 731 | ACAGCCTGCCTAAGAAGAAATGAAT |
| 286 | CATTCATTTCTTCTTAGGCAGGCTG | | 732 | CAGCCTGCCTAAGAAGAAATGAATG |
| 287 | ACATTCATTTCTTCTTAGGCAGGCT | | 733 | AGCCTGCCTAAGAAGAAATGAATGT |
| 288 | CACATTCATTTCTTCTTAGGCAGGC | | 734 | GCCTGCCTAAGAAGAAATGAATGTG |
| 289 | TCACATTCATTTCTTCTTAGGCAGG | | 735 | CCTGCCTAAGAAGAAATGAATGTGA |
| 290 | TTCACATTCATTTCTTCTTAGGCAG | | 736 | CTGCCTAAGAAGAAATGAATGTGAA |
| 291 | ATTCACATTCATTTCTTCTTAGGCA | | 737 | TGCCTAAGAAGAAATGAATGTGAAT |
| 292 | CATTCACATTCATTTCTTCTTAGGC | | 738 | GCCTAAGAAGAAATGAATGTGAATG |
| 293 | GCATTCACATTCATTTCTTCTTAGG | | 739 | CCTAAGAAGAAATGAATGTGAATGC |
| 294 | CGCATTCACATTCATTTCTTCTTAG | | 740 | CTAAGAAGAAATGAATGTGAATGCG |
| 295 | CCGCATTCACATTCATTTCTTCTTA | | 741 | TAAGAAGAAATGAATGTGAATGCGG |
| 296 | GCCGCATTCACATTCATTTCTTCTT | | 742 | AAGAAGAAATGAATGTGAATGCGGC |
| 297 | AGCCGCATTCACATTCATTTCTTCT | | 743 | AGAAGAAATGAATGTGAATGCGGCT |
| 298 | AAGCCGCATTCACATTCATTTCTTC | | 744 | GAAGAAATGAATGTGAATGCGGCTT |
| 299 | CAAGCCGCATTCACATTCATTTCTT | | 745 | AAGAAATGAATGTGAATGCGGCTTG |
| 300 | ACAAGCCGCATTCACATTCATTTCT | | 746 | AGAAATGAATGTGAATGCGGCTTGT |
| 301 | CACAAGCCGCATTCACATTCATTTC | | 747 | GAAATGAATGTGAATGCGGCTTGTG |
| 302 | CCACAAGCCGCATTCACATTCATTT | | 748 | AAATGAATGTGAATGCGGCTTGTGG |
| 303 | GCCACAAGCCGCATTCACATTCATT | | 749 | AATGAATGTGAATGCGGCTTGTGGC |
| 304 | TGCCACAAGCCGCATTCACATTCAT | | 750 | ATGAATGTGAATGCGGCTTGTGGCA |
| 305 | GTGCCACAAGCCGCATTCACATTCA | | 751 | TGAATGTGAATGCGGCTTGTGGCAC |
| 306 | TGTGCCACAAGCCGCATTCACATTC | | 752 | GAATGTGAATGCGGCTTGTGGCACA |
| 307 | CTGTGCCACAAGCCGCATTCACATT | | 753 | AATGTGAATGCGGCTTGTGGCACAG |
| 308 | ACTGTGCCACAAGCCGCATTCACAT | | 754 | ATGTGAATGCGGCTTGTGGCACAGT |
| 309 | AACTGTGCCACAAGCCGCATTCACA | | 755 | TGTGAATGCGGCTTGTGGCACAGTT |
| 310 | CAACTGTGCCACAAGCCGCATTCAC | | 756 | GTGAATGCGGCTTGTGGCACAGTTG |
| 311 | TCAACTGTGCCACAAGCCGCATTCA | | 757 | TGAATGCGGCTTGTGGCACAGTTGA |
| 312 | GTCAACTGTGCCACAAGCCGCATTC | | 758 | GAATGCGGCTTGTGGCACAGTTGAC |
| 313 | TGTCAACTGTGCCACAAGCCGCATT | | 759 | AATGCGGCTTGTGGCACAGTTGACA |
| 314 | TTGTCAACTGTGCCACAAGCCGCAT | | 760 | ATGCGGCTTGTGGCACAGTTGACAA |
| 315 | CTTGTCAACTGTGCCACAAGCCGCA | | 761 | TGCGGCTTGTGGCACAGTTGACAAG |
| 316 | CCTTGTCAACTGTGCCACAAGCCGC | | 762 | GCGGCTTGTGGCACAGTTGACAAGG |
| 317 | TCCTTGTCAACTGTGCCACAAGCCG | | 763 | CGGCTTGTGGCACAGTTGACAAGGA |
| 318 | ATCCTTGTCAACTGTGCCACAAGCC | | 764 | GGCTTGTGGCACAGTTGACAAGGAT |
| 319 | CATCCTTGTCAACTGTGCCACAAGC | | 765 | GCTTGTGGCACAGTTGACAAGGATG |
| 320 | TCATCCTTGTCAACTGTGCCACAAG | | 766 | CTTGTGGCACAGTTGACAAGGATGA |
| 321 | ATCATCCTTGTCAACTGTGCCACAA | | 767 | TTGTGGCACAGTTGACAAGGATGAT |
| 322 | TATCATCCTTGTCAACTGTGCCACA | | 768 | TGTGGCACAGTTGACAAGGATGATA |
| 323 | TTATCATCCTTGTCAACTGTGCCAC | | 769 | GTGGCACAGTTGACAAGGATGATAA |
| 324 | TTTATCATCCTTGTCAACTGTGCCA | | 770 | TGGCACAGTTGACAAGGATGATAAA |
| 325 | ATTTATCATCCTTGTCAACTGTGCC | | 771 | GGCACAGTTGACAAGGATGATAAAT |
| 326 | GATTTATCATCCTTGTCAACTGTGC | | 772 | GCACAGTTGACAAGGATGATAAATC |
| 327 | TGATTTATCATCCTTGTCAACTGTG | | 773 | CACAGTTGACAAGGATGATAAATCA |
| 328 | TTGATTTATCATCCTTGTCAACTGT | | 774 | ACAGTTGACAAGGATGATAAATCAA |
| 329 | ATTGATTTATCATCCTTGTCAACTG | | 775 | CAGTTGACAAGGATGATAAATCAAT |
| 330 | TATTGATTTATCATCCTTGTCAACT | | 776 | AGTTGACAAGGATGATAAATCAATA |
| 331 | TTATTGATTTATCATCCTTGTCAAC | | 777 | GTTGACAAGGATGATAAATCAATAA |
| 332 | ATTATTGATTTATCATCCTTGTCAA | | 778 | TTGACAAGGATGATAAATCAATAAT |
| 333 | CATTATTGATTTATCATCCTTGTCA | | 779 | TGACAAGGATGATAAATCAATAATG |
| 334 | GCATTATTGATTTATCATCCTTGTC | | 780 | GACAAGGATGATAAATCAATAATGC |
| 335 | TGCATTATTGATTTATCATCCTTGT | | 781 | ACAAGGATGATAAATCAATAATGCA |
| 336 | TTGCATTATTGATTTATCATCCTTG | | 782 | CAAGGATGATAAATCAATAATGCAA |
| 337 | CTTGCATTATTGATTTATCATCCTT | | 783 | AAGGATGATAAATCAATAATGCAAG |
| 338 | GCTTGCATTATTGATTTATCATCCT | | 784 | AGGATGATAAATCAATAATGCAAGC |
| 339 | AGCTTGCATTATTGATTTATCATCC | | 785 | GGATGATAAATCAATAATGCAAGCT |
| 340 | AAGCTTGCATTATTGATTTATCATC | | 786 | GATGATAAATCAATAATGCAAGCTT |
| 341 | TAAGCTTGCATTATTGATTTATCAT | | 787 | ATGATAAATCAATAATGCAAGCTTA |
| 342 | GTAAGCTTGCATTATTGATTTATCA | | 788 | TGATAAATCAATAATGCAAGCTTAC |
| 343 | AGTAAGCTTGCATTATTGATTTATC | | 789 | GATAAATCAATAATGCAAGCTTACT |
| 344 | TAGTAAGCTTGCATTATTGATTTAT | | 790 | ATAAATCAATAATGCAAGCTTACTA |
| 345 | ATAGTAAGCTTGCATTATTGATTTA | | 791 | TAAATCAATAATGCAAGCTTACTAT |
| 346 | GATAGTAAGCTTGCATTATTGATTT | | 792 | AAATCAATAATGCAAGCTTACTATC |
| 347 | TGATAGTAAGCTTGCATTATTGATT | | 793 | AATCAATAATGCAAGCTTACTATCA |
| 348 | ATGATAGTAAGCTTGCATTATTGAT | | 794 | ATCAATAATGCAAGCTTACTATCAT |
| 349 | AATGATAGTAAGCTTGCATTATTGA | | 795 | TCAATAATGCAAGCTTACTATCATT |
| 350 | AAATGATAGTAAGCTTGCATTATTG | | 796 | CAATAATGCAAGCTTACTATCATTT |
| 351 | TAAATGATAGTAAGCTTGCATTATT | | 797 | AATAATGCAAGCTTACTATCATTTA |
| 352 | ATAAATGATAGTAAGCTTGCATTAT | | 798 | ATAATGCAAGCTTACTATCATTTAT |
| 353 | CATAAATGATAGTAAGCTTGCATTA | | 799 | TAATGCAAGCTTACTATCATTTATG |
| 354 | TCATAAATGATAGTAAGCTTGCATT | | 800 | AATGCAAGCTTACTATCATTTATGA |
| 355 | TTCATAAATGATAGTAAGCTTGCAT | | 801 | ATGCAAGCTTACTATCATTTATGAA |
| 356 | ATTCATAAATGATAGTAAGCTTGCA | | 802 | TGCAAGCTTACTATCATTTATGAAT |
| 357 | TATTCATAAATGATAGTAAGCTTGC | | 803 | GCAAGCTTACTATCATTTATGAATA |
| 358 | CTATTCATAAATGATAGTAAGCTTG | | 804 | CAAGCTTACTATCATTTATGAATAG |
| 359 | GCTATTCATAAATGATAGTAAGCTT | | 805 | AAGCTTACTATCATTTATGAATAGC |
| 360 | TGCTATTCATAAATGATAGTAAGCT | | 806 | AGCTTACTATCATTTATGAATAGCA |
| 361 | TTGCTATTCATAAATGATAGTAAGC | | 807 | GCTTACTATCATTTATGAATAGCAA |
| 362 | ATTGCTATTCATAAATGATAGTAAG | | 808 | CTTACTATCATTTATGAATAGCAAT |
| 363 | TATTGCTATTCATAAATGATAGTAA | | 809 | TTACTATCATTTATGAATAGCAATA |
| 364 | GTATTGCTATTCATAAATGATAGTA | | 810 | TACTATCATTTATGAATAGCAATAC |
| 365 | AGTATTGCTATTCATAAATGATAGT | | 811 | ACTATCATTTATGAATAGCAATACT |
| 366 | CAGTATTGCTATTCATAAATGATAG | | 812 | CTATCATTTATGAATAGCAATACTG |
| 367 | TCAGTATTGCTATTCATAAATGATA | | 813 | TATCATTTATGAATAGCAATACTGA |
| 368 | TTCAGTATTGCTATTCATAAATGAT | | 814 | ATCATTTATGAATAGCAATACTGAA |
| 369 | CTTCAGTATTGCTATTCATAAATGA | | 815 | TCATTTATGAATAGCAATACTGAAG |
| 370 | TCTTCAGTATTGCTATTCATAAATG | | 816 | CATTTATGAATAGCAATACTGAAGA |
| 371 | TTCTTCAGTATTGCTATTCATAAAT | | 817 | ATTTATGAATAGCAATACTGAAGAA |
| 372 | TTTCTTCAGTATTGCTATTCATAAA | | 818 | TTTATGAATAGCAATACTGAAGAAA |
| 373 | ATTTCTTCAGTATTGCTATTCATAA | | 819 | TTATGAATAGCAATACTGAAGAAAT |
| 374 | AATTTCTTCAGTATTGCTATTCATA | | 820 | TATGAATAGCAATACTGAAGAAATT |
| 375 | TAATTTCTTCAGTATTGCTATTCAT | | 821 | ATGAATAGCAATACTGAAGAAATTA |
| 376 | TTAATTTCTTCAGTATTGCTATTCA | | 822 | TGAATAGCAATACTGAAGAAATTAA |
| 377 | TTTAATTTCTTCAGTATTGCTATTC | polyA | 823 | GAATAGCAATACTGAAGAAATTAAA |
| | | signal | | |
| 378 | TTTTAATTTCTTCAGTATTGCTATT | polyA | 824 | AATAGCAATACTGAAGAAATTAAAA |
| | | signal | | |
| 379 | GTTTTAATTTCTTCAGTATTGCTAT | polyA | 825 | ATAGCAATACTGAAGAAATTAAAAC |
| | | signal | | |
| 380 | TGTTTTAATTTCTTCAGTATTGCTA | poly A | 826 | TAGCAATACTGAAGAAATTAAAACA |
| | | signal | | |
| 381 | TTGTTTTAATTTCTTCAGTATTGCT | polyA | 827 | AGCAATACTGAAGAAATTAAAACAA |
| | | signal | | |
| 382 | TTTGTTTTAATTTCTTCAGTATTGC | polyA | 828 | GCAATACTGAAGAAATTAAAACAAA |
| | | signal | | |
| 383 | TTTTGTTTTAATTTCTTCAGTATTG | polyA | 829 | CAATACTGAAGAAATTAAAACAAAA |
| | | signal | | |
| 384 | CTTTTGTTTTAATTTCTTCAGTATT | poly A | 830 | AATACTGAAGAAATTAAAACAAAAG |
| | | signal | | |
| 385 | TCTTTTGTTTTAATTTCTTCAGTAT | polyA | 831 | ATACTGAAGAAATTAAAACAAAAGA |
| | | signal | | |
| 386 | ATCTTTTGTTTTAATTTCTTCAGTA | polyA | 832 | TACTGAAGAAATTAAAACAAAAGAT |
| | | signal | | |
| 387 | AATCTTTTGTTTTAATTTCTTCAGT | polyA | 833 | ACTGAAGAAATTAAAACAAAAGATT |
| | | signal | | |
| 388 | CAATCTTTTGTTTTAATTTCTTCAG | polyA | 834 | CTGAAGAAATTAAAACAAAAGATTG |
| | | signal | | |
| 389 | GCAATCTTTTGTTTTAATTTCTTCA | poly A | 835 | TGAAGAAATTAAAACAAAAGATTGC |
| | | signal | | |
| 390 | AGCAATCTTTTGTTTTAATTTCTTC | polyA | 836 | GAAGAAATTAAAACAAAAGATTGCT |
| | | signal | | |
| 391 | CAGCAATCTTTTGTTTTAATTTCTT | polyA | 837 | AAGAAATTAAAACAAAAGATTGCTG |
| | | signal | | |
| 392 | ACAGCAATCTTTTGTTTTAATTTCT | polyA | 838 | AGAAATTAAAACAAAAGATTGCTGT |
| | | signal | | |
| 393 | GACAGCAATCTTTTGTTTTAATTTC | poly A | 839 | GAAATTAAAACAAAAGATTGCTGTC |
| | | signal | | |
| 394 | AGACAGCAATCTTTTGTTTTAATTT | polyA | 840 | AAATTAAAACAAAAGATTGCTGTCT |
| | | signal | | |
| 395 | GAGACAGCAATCTTTTGTTTTAATT | polyA | 841 | AATTAAAACAAAAGATTGCTGTCTC |
| | | signal | | |
| | | and site | | |
| 396 | TGAGACAGCAATCTTTTGTTTTAAT | polyA | 842 | ATTAAAACAAAAGATTGCTGTCTCA |
| | | signal | | |
| | | and site | | |
| 397 | TTGAGACAGCAATCTTTTGTTTTAA | polyA | 843 | TTAAAACAAAAGATTGCTGTCTCAA |
| | | site | | |
| 398 | ATTGAGACAGCAATCTTTTGTTTTA | poly A | 844 | TAAAACAAAAGATTGCTGTCTCAAT |
| | | site | | |
| 399 | TATTGAGACAGCAATCTTTTGTTTT | polyA | 845 | AAAACAAAAGATTGCTGTCTCAATA |
| | | site | | |
| 400 | ATATTGAGACAGCAATCTTTTGTTT | polyA | 846 | AAACAAAAGATTGCTGTCTCAATAT |
| | | site | | |
| 401 | TATATTGAGACAGCAATCTTTTGTT | polyA | 847 | AACAAAAGATTGCTGTCTCAATATA |
| | | site | | |
| 402 | ATATATTGAGACAGCAATCTTTTGT | polyA | 848 | ACAAAAGATTGCTGTCTCAATATAT |
| | | site | | |
| 403 | GATATATTGAGACAGCAATCTTTTG | polyA | 849 | CAAAAGATTGCTGTCTCAATATATC |
| | | site | | |
| 404 | AGATATATTGAGACAGCAATCTTTT | poly A | 850 | AAAAGATTGCTGTCTCAATATATCT |
| | | site | | |
| 405 | AAGATATATTGAGACAGCAATCTTT | polyA | 851 | AAAGATTGCTGTCTCAATATATCTT |
| | | site | | |
| 406 | TAAGATATATTGAGACAGCAATCTT | polyA | 852 | AAGATTGCTGTCTCAATATATCTTA |
| | | site | | |
| 407 | ATAAGATATATTGAGACAGCAATCT | polyA | 853 | AGATTGCTGTCTCAATATATCTTAT |
| | | site | | |
| 408 | TATAAGATATATTGAGACAGCAATC | polyA | 854 | GATTGCTGTCTCAATATATCTTATA |
| | | site | | |
| 409 | ATATAAGATATATTGAGACAGCAAT | polyA | 855 | ATTGCTGTCTCAATATATCTTATAT |
| | | site | | |
| 410 | AATATAAGATATATTGAGACAGCAA | poly A | 856 | TTGCTGTCTCAATATATCTTATATT |
| | | site | | |
| 411 | AAATATAAGATATATTGAGACAGCA | poly A | 857 | TGCTGTCTCAATATATCTTATATTT |
| | | site | | |
| 412 | TAAATATAAGATATATTGAGACAGC | polyA | 858 | GCTGTCTCAATATATCTTATATTTA |
| | | site | | |
| 413 | ATAAATATAAGATATATTGAGACAG | | 859 | CTGTCTCAATATATCTTATATTTAT |
| 414 | AATAAATATAAGATATATTGAGACA | | 860 | TGTCTCAATATATCTTATATTTATT |
| 415 | TAATAAATATAAGATATATTGAGAC | | 861 | GTCTCAATATATCTTATATTTATTA |
| 416 | ATAATAAATATAAGATATATTGAGA | | 862 | TCTCAATATATCTTATATTTATTAT |
| 417 | AATAATAAATATAAGATATATTGAG | | 863 | CTCAATATATCTTATATTTATTATT |
| 418 | AAATAATAAATATAAGATATATTGA | | 864 | TCAATATATCTTATATTTATTATTT |
| 419 | TAAATAATAAATATAAGATATATTG | | 865 | CAATATATCTTATATTTATTATTTA |
| 420 | GTAAATAATAAATATAAGATATATT | | 866 | AATATATCTTATATTTATTATTTAC |
| 421 | GGTAAATAATAAATATAAGATATAT | | 867 | ATATATCTTATATTTATTATTTACC |
| 422 | TGGTAAATAATAAATATAAGATATA | | 868 | TATATCTTATATTTATTATTTACCA |
| 423 | TTGGTAAATAATAAATATAAGATAT | | 869 | ATATCTTATATTTATTATTTACCAA |
| 424 | TTTGGTAAATAATAAATATAAGATA | | 870 | TATCTTATATTTATTATTTACCAAA |
| 425 | ATTTGGTAAATAATAAATATAAGAT | | 871 | ATCTTATATTTATTATTTACCAAAT |
| 426 | AATTTGGTAAATAATAAATATAAGA | | 872 | TCTTATATTTATTATTTACCAAATT |
| 427 | TAATTTGGTAAATAATAAATATAAG | | 873 | CTTATATTTATTATTTACCAAATTA |
| 428 | ATAATTTGGTAAATAATAAATATAA | | 874 | TTATATTTATTATTTACCAAATTAT |
| 429 | AATAATTTGGTAAATAATAAATATA | | 875 | TATATTTATTATTTACCAAATTATT |
| 430 | GAATAATTTGGTAAATAATAAATAT | | 876 | ATATTTATTATTTACCAAATTATTC |
| 431 | AGAATAATTTGGTAAATAATAAATA | | 877 | TATTTATTATTTACCAAATTATTCT |
| 432 | TAGAATAATTTGGTAAATAATAAAT | | 878 | ATTTATTATTTACCAAATTATTCTA |
| 433 | TTAGAATAATTTGGTAAATAATAAA | | 879 | TTTATTATTTACCAAATTATTCTAA |
| 434 | CTTAGAATAATTTGGTAAATAATAA | | 880 | TTATTATTTACCAAATTATTCTAAG |
| 435 | TCTTAGAATAATTTGGTAAATAATA | | 881 | TATTATTTACCAAATTATTCTAAGA |
| 436 | CTCTTAGAATAATTTGGTAAATAAT | | 882 | ATTATTTACCAAATTATTCTAAGAG |
| 437 | ACTCTTAGAATAATTTGGTAAATAA | | 883 | TTATTTACCAAATTATTCTAAGAGT |
| 438 | TACTCTTAGAATAATTTGGTAAATA | | 884 | TATTTACCAAATTATTCTAAGAGTA |
| 439 | ATACTCTTAGAATAATTTGGTAAAT | | 885 | ATTTACCAAATTATTCTAAGAGTAT |
| 440 | AATACTCTTAGAATAATTTGGTAAA | | 886 | TTTACCAAATTATTCTAAGAGTATT |
| 441 | AAATACTCTTAGAATAATTTGGTAA | | 887 | TTACCAAATTATTCTAAGAGTATTT |
| 442 | GAAATACTCTTAGAATAATTTGGTA | | 888 | TACCAAATTATTCTAAGAGTATTTC |
| 443 | AGAAATACTCTTAGAATAATTTGGT | | 889 | ACCAAATTATTCTAAGAGTATTTCT |
| 444 | AAGAAATACTCTTAGAATAATTTGG | | 890 | CCAAATTATTCTAAGAGTATTTCTT |
| 445 | GAAGAAATACTCTTAGAATAATTTG | | 891 | CAAATTATTCTAAGAGTATTTCTTC |
| 446 | GGAAGAAATACTCTTAGAATAATTT | | 892 | AAATTATTCTAAGAGTATTTCTTCC |

* At least one (i.e., one or more) nucleoside linkage of the oligonucleotide sequence is independently selected from a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate (e.g., comprising a phosphorodiamidate morpholino (PMO), 3' amino ribose, or 5' amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

Table 2 below identifies additional STMN2 AON sequences:

TABLE 2
Additional STMN2 AON Sequences (corresponding to
SEQ ID NOs: 1-446 but with thymine bases
replaced with uracil bases)

| SEQ ID NO: | AON Sequence* (5′→3′) |
|---|---|
| 893 | GGAGGGAUACCUGUAUAUUACAAGU |
| 894 | AGGAGGGAUACCUGUAUAUUACAAG |
| 895 | CAGGAGGGAUACCUGUAUAUUACAA |
| 896 | CCAGGAGGGAUACCUGUAUAUUACA |
| 897 | ACCAGGAGGGAUACCUGUAUAUUAC |
| 898 | UACCAGGAGGGAUACCUGUAUAUUA |
| 899 | UUACCAGGAGGGAUACCUGUAUAUU |
| 900 | CUUACCAGGAGGGAUACCUGUAUAU |
| 901 | GCUUACCAGGAGGGAUACCUGUAUA |
| 902 | AGCUUACCAGGAGGGAUACCUGUAU |
| 903 | GAGCUUACCAGGAGGGAUACCUGUA |
| 904 | AGAGCUUACCAGGAGGGAUACCUGU |
| 905 | CAGAGCUUACCAGGAGGGAUACCUG |
| 906 | CCAGAGCUUACCAGGAGGGAUACCU |
| 907 | ACCAGAGCUUACCAGGAGGGAUACC |
| 908 | UACCAGAGCUUACCAGGAGGGAUAC |
| 909 | AUACCAGAGCUUACCAGGAGGGAUA |
| 910 | AAUACCAGAGCUUACCAGGAGGGAU |
| 911 | UAAUACCAGAGCUUACCAGGAGGGA |
| 912 | AUAAUACCAGAGCUUACCAGGAGGG |
| 913 | CAUAAUACCAGAGCUUACCAGGAGG |
| 914 | ACAUAAUACCAGAGCUUACCAGGAG |
| 915 | GACAUAAUACCAGAGCUUACCAGGA |
| 916 | AGACAUAAUACCAGAGCUUACCAGG |
| 917 | AAGACAUAAUACCAGAGCUUACCAG |
| 918 | UAAGACAUAAUACCAGAGCUUACCA |
| 919 | UUAAGACAUAAUACCAGAGCUUACC |
| 920 | GUUAAGACAUAAUACCAGAGCUUAC |
| 921 | UGUUAAGACAUAAUACCAGAGCUUA |
| 922 | AUGUUAAGACAUAAUACCAGAGCUU |
| 923 | AAUGUUAAGACAUAAUACCAGAGCU |
| 924 | AAAUGUUAAGACAUAAUACCAGAGC |
| 925 | AAAAUGUUAAGACAUAAUACCAGAG |
| 926 | AAAAAUGUUAAGACAUAAUACCAGA |
| 927 | UAAAAAUGUUAAGACAUAAUACCAG |
| 928 | UUAAAAAUGUUAAGACAUAAUACCA |
| 929 | UUUAAAAAUGUUAAGACAUAAUACC |
| 930 | AUUUAAAAAUGUUAAGACAUAAUAC |
| 931 | GAUUUAAAAAUGUUAAGACAUAAUA |
| 932 | AGAUUUAAAAAUGUUAAGACAUAAU |
| 933 | UAGAUUUAAAAAUGUUAAGACAUAA |
| 934 | AUAGAUUUAAAAAUGUUAAGACAUA |
| 935 | CAUAGAUUUAAAAAUGUUAAGACAU |
| 936 | CCAUAGAUUUAAAAAUGUUAAGACA |
| 937 | ACCAUAGAUUUAAAAAUGUUAAGAC |
| 938 | UACCAUAGAUUUAAAAAUGUUAAGA |
| 939 | UUACCAUAGAUUUAAAAAUGUUAAG |
| 940 | AUUACCAUAGAUUUAAAAAUGUUAA |
| 941 | GAUUACCAUAGAUUUAAAAAUGUUA |
| 942 | AGAUUACCAUAGAUUUAAAAAUGUU |
| 943 | AAGAUUACCAUAGAUUUAAAAAUGU |
| 944 | AAAGAUUACCAUAGAUUUAAAAAUG |
| 945 | UAAAGAUUACCAUAGAUUUAAAAAU |
| 946 | GUAAAGAUUACCAUAGAUUUAAAAA |
| 947 | UGUAAAGAUUACCAUAGAUUUAAAA |
| 948 | UUGUAAAGAUUACCAUAGAUUUAAA |
| 949 | UUUGUAAAGAUUACCAUAGAUUUAA |
| 950 | UUUUGUAAAGAUUACCAUAGAUUUA |
| 951 | AUUUUGUAAAGAUUACCAUAGAUUU |
| 952 | UAUUUUGUAAAGAUUACCAUAGAUU |
| 953 | AUAUUUUGUAAAGAUUACCAUAGAU |
| 954 | AAUAUUUUGUAAAGAUUACCAUAGA |
| 955 | AAAUAUUUUGUAAAGAUUACCAUAG |
| 956 | AAAAUAUUUUGUAAAGAUUACCAUA |
| 957 | UAAAAUAUUUUGUAAAGAUUACCAU |
| 958 | GUAAAAUAUUUUGUAAAGAUUACCA |
| 959 | AGUAAAAUAUUUUGUAAAGAUUACC |
| 960 | AAGUAAAAUAUUUUGUAAAGAUUAC |
| 961 | GAAGUAAAAUAUUUUGUAAAGAUUA |
| 962 | GGAAGUAAAAUAUUUUGUAAAGAUU |
| 963 | CGGAAGUAAAAUAUUUUGUAAAGAU |
| 964 | UCGGAAGUAAAAUAUUUUGUAAAGA |
| 965 | UUCGGAAGUAAAAUAUUUUGUAAAG |
| 966 | GUUCGGAAGUAAAAUAUUUUGUAAA |
| 967 | AGUUCGGAAGUAAAAUAUUUUGUAA |
| 968 | GAGUUCGGAAGUAAAAUAUUUUGUA |
| 969 | UGAGUUCGGAAGUAAAAUAUUUUGU |
| 970 | AUGAGUUCGGAAGUAAAAUAUUUUG |
| 971 | UAUGAGUUCGGAAGUAAAAUAUUUU |
| 972 | AUAUGAGUUCGGAAGUAAAAUAUUU |
| 973 | UAUAUGAGUUCGGAAGUAAAAUAUU |
| 974 | GUAUAUGAGUUCGGAAGUAAAAUAU |
| 975 | GGUAUAUGAGUUCGGAAGUAAAAUA |
| 976 | AGGUAUAUGAGUUCGGAAGUAAAAU |
| 977 | CAGGUAUAUGAGUUCGGAAGUAAAA |
| 978 | CCAGGUAUAUGAGUUCGGAAGUAAA |
| 979 | CCCAGGUAUAUGAGUUCGGAAGUAA |
| 980 | CCCCAGGUAUAUGAGUUCGGAAGUA |
| 981 | UCCCCAGGUAUAUGAGUUCGGAAGU |
| 982 | AUCCCCAGGUAUAUGAGUUCGGAAG |
| 983 | AAUCCCCAGGUAUAUGAGUUCGGAA |
| 984 | AAAUCCCCAGGUAUAUGAGUUCGGA |
| 985 | AAAAUCCCCAGGUAUAUGAGUUCGG |
| 986 | UAAAAUCCCCAGGUAUAUGAGUUCG |
| 987 | AUAAAAUCCCCAGGUAUAUGAGUUC |
| 988 | AAUAAAAUCCCCAGGUAUAUGAGUU |
| 989 | UAAUAAAAUCCCCAGGUAUAUGAGU |
| 990 | GUAAUAAAAUCCCCAGGUAUAUGAG |
| 991 | AGUAAUAAAAUCCCCAGGUAUAUGA |
| 992 | GAGUAAUAAAAUCCCCAGGUAUAUG |
| 993 | AGAGUAAUAAAAUCCCCAGGUAUAU |
| 994 | CAGAGUAAUAAAAUCCCCAGGUAUA |
| 995 | CCAGAGUAAUAAAAUCCCCAGGUAU |
| 996 | CCCAGAGUAAUAAAAUCCCCAGGUA |
| 997 | UCCCAGAGUAAUAAAAUCCCCAGGU |
| 998 | UUCCCAGAGUAAUAAAAUCCCCAGG |
| 999 | AUUCCCAGAGUAAUAAAAUCCCCAG |
| 1000 | AAUUCCCAGAGUAAUAAAAUCCCCA |
| 1001 | UAAUUCCCAGAGUAAUAAAAUCCCC |
| 1002 | AUAAUUCCCAGAGUAAUAAAAUCCC |
| 1003 | CAUAAUUCCCAGAGUAAUAAAAUCC |
| 1004 | ACAUAAUUCCCAGAGUAAUAAAAUC |
| 1005 | CACAUAAUUCCCAGAGUAAUAAAAU |
| 1006 | ACACAUAAUUCCCAGAGUAAUAAAA |
| 1007 | AACACAUAAUUCCCAGAGUAAUAAA |
| 1008 | GAACACAUAAUUCCCAGAGUAAUAA |
| 1009 | AGAACACAUAAUUCCCAGAGUAAUA |
| 1010 | CAGAACACAUAAUUCCCAGAGUAAU |
| 1011 | GCAGAACACAUAAUUCCCAGAGUAA |
| 1012 | GGCAGAACACAUAAUUCCCAGAGUA |
| 1013 | GGGCAGAACACAUAAUUCCCAGAGU |
| 1014 | GGGGCAGAACACAUAAUUCCCAGAG |
| 1015 | UGGGGCAGAACACAUAAUUCCCAGA |
| 1016 | AUGGGGCAGAACACAUAAUUCCCAG |
| 1017 | GAUGGGGCAGAACACAUAAUUCCCA |
| 1018 | UGAUGGGGCAGAACACAUAAUUCCC |
| 1019 | GUGAUGGGGCAGAACACAUAAUUCC |
| 1020 | AGUGAUGGGGCAGAACACAUAAUUC |
| 1021 | GAGUGAUGGGGCAGAACACAUAAUU |
| 1022 | AGAGUGAUGGGGCAGAACACAUAAU |
| 1023 | GAGAGUGAUGGGGCAGAACACAUAA |
| 1024 | AGAGAGUGAUGGGGCAGAACACAUA |
| 1025 | GAGAGAGUGAUGGGGCAGAACACAU |
| 1026 | AGAGAGAGUGAUGGGGCAGAACACA |
| 1027 | AAGAGAGAGUGAUGGGGCAGAACAC |
| 1028 | UAAGAGAGAGUGAUGGGGCAGAACA |
| 1029 | UUAAGAGAGAGUGAUGGGGCAGAAC |
| 1030 | AUUAAGAGAGAGUGAUGGGGCAGAA |
| 1031 | AAUUAAGAGAGAGUGAUGGGGCAGA |
| 1032 | CAAUUAAGAGAGAGUGAUGGGGCAG |
| 1033 | CCAAUUAAGAGAGAGUGAUGGGGCA |
| 1034 | UCCAAUUAAGAGAGAGUGAUGGGGC |
| 1035 | AUCCAAUUAAGAGAGAGUGAUGGGG |
| 1036 | AAUCCAAUUAAGAGAGAGUGAUGGG |
| 1037 | AAAUCCAAUUAAGAGAGAGUGAUGG |
| 1038 | AAAAUCCAAUUAAGAGAGAGUGAUG |
| 1039 | AAAAAUCCAAUUAAGAGAGAGUGAU |
| 1040 | UAAAAAUCCAAUUAAGAGAGAGUGA |
| 1041 | UUAAAAAUCCAAUUAAGAGAGAGUG |
| 1042 | UUUAAAAAUCCAAUUAAGAGAGAGU |
| 1043 | UUUUAAAAAUCCAAUUAAGAGAGAG |
| 1044 | AUUUUAAAAAUCCAAUUAAGAGAGA |
| 1045 | AAUUUUAAAAAUCCAAUUAAGAGAG |
| 1046 | UAAUUUUAAAAAUCCAAUUAAGAGA |
| 1047 | AUAAUUUUAAAAAUCCAAUUAAGAG |
| 1048 | UAUAAUUUUAAAAAUCCAAUUAAGA |
| 1049 | AUAUAAUUUUAAAAAUCCAAUUAAG |
| 1050 | AAUAUAAUUUUAAAAAUCCAAUUAA |
| 1051 | GAAUAUAAUUUUAAAAAUCCAAUUA |
| 1052 | UGAAUAUAAUUUUAAAAAUCCAAUU |
| 1053 | AUGAAUAUAAUUUUAAAAAUCCAAU |
| 1054 | UAUGAAUAUAAUUUUAAAAAUCCAA |
| 1055 | AUAUGAAUAUAAUUUUAAAAAUCCA |
| 1056 | AAUAUGAAUAUAAUUUUAAAAAUCC |
| 1057 | CAAUAUGAAUAUAAUUUUAAAAAUC |
| 1058 | GCAAUAUGAAUAUAAUUUUAAAAAU |
| 1059 | UGCAAUAUGAAUAUAAUUUUAAAAA |
| 1060 | CUGCAAUAUGAAUAUAAUUUUAAAA |
| 1061 | CCUGCAAUAUGAAUAUAAUUUUAAA |
| 1062 | UCCUGCAAUAUGAAUAUAAUUUUAA |
| 1063 | GUCCUGCAAUAUGAAUAUAAUUUUA |
| 1064 | AGUCCUGCAAUAUGAAUAUAAUUUU |
| 1065 | GAGUCCUGCAAUAUGAAUAUAAUUU |
| 1066 | CGAGUCCUGCAAUAUGAAUAUAAUU |
| 1067 | CCGAGUCCUGCAAUAUGAAUAUAAU |
| 1068 | GCCGAGUCCUGCAAUAUGAAUAUAA |
| 1069 | UGCCGAGUCCUGCAAUAUGAAUAUA |
| 1070 | CUGCCGAGUCCUGCAAUAUGAAUAU |
| 1071 | UCUGCCGAGUCCUGCAAUAUGAAUA |
| 1072 | UUCUGCCGAGUCCUGCAAUAUGAAU |
| 1073 | CUUCUGCCGAGUCCUGCAAUAUGAA |
| 1074 | UCUUCUGCCGAGUCCUGCAAUAUGA |
| 1075 | GUCUUCUGCCGAGUCCUGCAAUAUG |
| 1076 | GGUCUUCUGCCGAGUCCUGCAAUAU |
| 1077 | AGGUCUUCUGCCGAGUCCUGCAAUA |
| 1078 | AAGGUCUUCUGCCGAGUCCUGCAAU |
| 1079 | GAAGGUCUUCUGCCGAGUCCUGCAA |
| 1080 | CGAAGGUCUUCUGCCGAGUCCUGCA |
| 1081 | UCGAAGGUCUUCUGCCGAGUCCUGC |
| 1082 | CUCGAAGGUCUUCUGCCGAGUCCUG |
| 1083 | UCUCGAAGGUCUUCUGCCGAGUCCU |
| 1084 | CUCUCGAAGGUCUUCUGCCGAGUCC |
| 1085 | UCUCUCGAAGGUCUUCUGCCGAGUC |
| 1086 | UUCUCUCGAAGGUCUUCUGCCGAGU |
| 1087 | UUUCUCUCGAAGGUCUUCUGCCGAG |
| 1088 | CUUUCUCUCGAAGGUCUUCUGCCGA |
| 1089 | CCUUUCUCUCGAAGGUCUUCUGCCG |
| 1090 | ACCUUUCUCUCGAAGGUCUUCUGCC |
| 1091 | UACCUUUCUCUCGAAGGUCUUCUGC |
| 1092 | CUACCUUUCUCUCGAAGGUCUUCUG |
| 1093 | UCUACCUUUCUCUCGAAGGUCUUCU |
| 1094 | UUCUACCUUUCUCUCGAAGGUCUUC |
| 1095 | UUUCUACCUUUCUCUCGAAGGUCUU |
| 1096 | UUUUCUACCUUUCUCUCGAAGGUCU |
| 1097 | AUUUUCUACCUUUCUCUCGAAGGUC |
| 1098 | UAUUUUCUACCUUUCUCUCGAAGGU |
| 1099 | UUAUUUUCUACCUUUCUCUCGAAGG |
| 1100 | CUUAUUUUCUACCUUUCUCUCGAAG |
| 1101 | UCUUAUUUUCUACCUUUCUCUCGAA |
| 1102 | UUCUUAUUUUCUACCUUUCUCUCGA |
| 1103 | AUUCUUAUUUUCUACCUUUCUCUCG |
| 1104 | AAUUCUUAUUUUCUACCUUUCUCUC |
| 1105 | AAAUUCUUAUUUUCUACCUUUCUCU |
| 1106 | CAAAUUCUUAUUUUCUACCUUUCUC |
| 1107 | CCAAAUUCUUAUUUUCUACCUUUCU |
| 1108 | GCCAAAUUCUUAUUUUCUACCUUUC |
| 1109 | AGCCAAAUUCUUAUUUUCUACCUUU |
| 1110 | GAGCCAAAUUCUUAUUUUCUACCUU |
| 1111 | AGAGCCAAAUUCUUAUUUUCUACCU |
| 1112 | GAGAGCCAAAUUCUUAUUUUCUACC |
| 1113 | AGAGAGCCAAAUUCUUAUUUUCUAC |
| 1114 | CAGAGAGCCAAAUUCUUAUUUUCUA |
| 1115 | ACAGAGAGCCAAAUUCUUAUUUUCU |
| 1116 | CACAGAGAGCCAAAUUCUUAUUUUC |
| 1117 | ACACAGAGAGCCAAAUUCUUAUUUU |
| 1118 | CACACAGAGAGCCAAAUUCUUAUUU |
| 1119 | UCACACAGAGAGCCAAAUUCUUAUU |
| 1120 | CUCACACAGAGAGCCAAAUUCUUAU |
| 1121 | GCUCACACAGAGAGCCAAAUUCUUA |
| 1122 | UGCUCACACAGAGAGCCAAAUUCUU |
| 1123 | AUGCUCACACAGAGAGCCAAAUUCU |
| 1124 | CAUGCUCACACAGAGAGCCAAAUUC |
| 1125 | ACAUGCUCACACAGAGAGCCAAAUU |
| 1126 | CACAUGCUCACACAGAGAGCCAAAU |
| 1127 | ACACAUGCUCACACAGAGAGCCAAA |
| 1128 | CACACAUGCUCACACAGAGAGCCAA |
| 1129 | GCACACAUGCUCACACAGAGAGCCA |
| 1130 | CGCACACAUGCUCACACAGAGAGCC |
| 1131 | ACGCACACAUGCUCACACAGAGAGC |
| 1132 | CACGCACACAUGCUCACACAGAGAG |
| 1133 | ACACGCACACAUGCUCACACAGAGA |
| 1134 | CACACGCACACAUGCUCACACAGAG |
| 1135 | ACACACGCACACAUGCUCACACAGA |
| 1136 | CACACACGCACACAUGCUCACACAG |
| 1137 | GCACACACGCACACAUGCUCACACA |
| 1138 | CGCACACACGCACACAUGCUCACAC |
| 1139 | UCGCACACACGCACACAUGCUCACA |
| 1140 | CUCGCACACACGCACACAUGCUCAC |
| 1141 | UCUCGCACACACGCACACAUGCUCA |
| 1142 | CUCUCGCACACACGCACACAUGCUC |
| 1143 | UCUCUCGCACACACGCACACAUGCU |
| 1144 | CUCUCUCGCACACACGCACACAUGC |
| 1145 | UCUCUCUCGCACACACGCACACAUG |
| 1146 | CUCUCUCUCGCACACACGCACACAU |
| 1147 | UCUCUCUCUCGCACACACGCACACA |
| 1148 | CUCUCUCUCUCGCACACACGCACAC |
| 1149 | UCUCUCUCUCUCGCACACACGCACA |
| 1150 | GUCUCUCUCUCUCGCACACACGCAC |
| 1151 | UGUCUCUCUCUCUCGCACACACGCA |
| 1152 | CUGUCUCUCUCUCUCGCACACACGC |
| 1153 | UCUGUCUCUCUCUCUCGCACACACG |
| 1154 | GUCUGUCUCUCUCUCUCGCACACAC |
| 1155 | UGUCUGUCUCUCUCUCUCGCACACA |
| 1156 | CUGUCUGUCUCUCUCUCUCGCACAC |
| 1157 | GCUGUCUGUCUCUCUCUCUCGCACA |
| 1158 | GGCUGUCUGUCUCUCUCUCUCGCAC |
| 1159 | AGGCUGUCUGUCUCUCUCUCUCGCA |
| 1160 | CAGGCUGUCUGUCUCUCUCUCUCGC |
| 1161 | GCAGGCUGUCUGUCUCUCUCUCUCG |
| 1162 | GGCAGGCUGUCUGUCUCUCUCUCUC |
| 1163 | AGGCAGGCUGUCUGUCUCUCUCUCU |
| 1164 | UAGGCAGGCUGUCUGUCUCUCUCUC |
| 1165 | UUAGGCAGGCUGUCUGUCUCUCUCU |
| 1166 | CUUAGGCAGGCUGUCUGUCUCUCUC |
| 1167 | UCUUAGGCAGGCUGUCUGUCUCUCU |
| 1168 | UUCUUAGGCAGGCUGUCUGUCUCUC |
| 1169 | CUUCUUAGGCAGGCUGUCUGUCUCU |
| 1170 | UCUUCUUAGGCAGGCUGUCUGUCUC |
| 1171 | UUCUUCUUAGGCAGGCUGUCUGUCU |
| 1172 | UUUCUUCUUAGGCAGGCUGUCUGUC |
| 1173 | AUUUCUUCUUAGGCAGGCUGUCUGU |
| 1174 | CAUUUCUUCUUAGGCAGGCUGUCUG |
| 1175 | UCAUUUCUUCUUAGGCAGGCUGUCU |
| 1176 | UUCAUUUCUUCUUAGGCAGGCUGUC |
| 1177 | AUUCAUUUCUUCUUAGGCAGGCUGU |
| 1178 | CAUUCAUUUCUUCUUAGGCAGGCUG |
| 1179 | ACAUUCAUUUCUUCUUAGGCAGGCU |
| 1180 | CACAUUCAUUUCUUCUUAGGCAGGC |
| 1181 | UCACAUUCAUUUCUUCUUAGGCAGG |
| 1182 | UUCACAUUCAUUUCUUCUUAGGCAG |
| 1183 | AUUCACAUUCAUUUCUUCUUAGGCA |
| 1184 | CAUUCACAUUCAUUUCUUCUUAGGC |
| 1185 | GCAUUCACAUUCAUUUCUUCUUAGG |
| 1186 | CGCAUUCACAUUCAUUUCUUCUUAG |
| 1187 | CCGCAUUCACAUUCAUUUCUUCUUA |
| 1188 | GCCGCAUUCACAUUCAUUUCUUCUU |
| 1189 | AGCCGCAUUCACAUUCAUUUCUUCU |
| 1190 | AAGCCGCAUUCACAUUCAUUUCUUC |
| 1191 | CAAGCCGCAUUCACAUUCAUUUCUU |
| 1192 | ACAAGCCGCAUUCACAUUCAUUUCU |
| 1193 | CACAAGCCGCAUUCACAUUCAUUUC |
| 1194 | CCACAAGCCGCAUUCACAUUCAUUU |
| 1195 | GCCACAAGCCGCAUUCACAUUCAUU |
| 1196 | UGCCACAAGCCGCAUUCACAUUCAU |
| 1197 | GUGCCACAAGCCGCAUUCACAUUCA |
| 1198 | UGUGCCACAAGCCGCAUUCACAUUC |
| 1199 | CUGUGCCACAAGCCGCAUUCACAUU |
| 1200 | ACUGUGCCACAAGCCGCAUUCACAU |
| 1201 | AACUGUGCCACAAGCCGCAUUCACA |
| 1202 | CAACUGUGCCACAAGCCGCAUUCAC |
| 1203 | UCAACUGUGCCACAAGCCGCAUUCA |
| 1204 | GUCAACUGUGCCACAAGCCGCAUUC |
| 1205 | UGUCAACUGUGCCACAAGCCGCAUU |
| 1206 | UUGUCAACUGUGCCACAAGCCGCAU |
| 1207 | CUUGUCAACUGUGCCACAAGCCGCA |
| 1208 | CCUUGUCAACUGUGCCACAAGCCGC |
| 1209 | UCCUUGUCAACUGUGCCACAAGCCG |
| 1210 | AUCCUUGUCAACUGUGCCACAAGCC |
| 1211 | CAUCCUUGUCAACUGUGCCACAAGC |
| 1212 | UCAUCCUUGUCAACUGUGCCACAAG |
| 1213 | AUCAUCCUUGUCAACUGUGCCACAA |
| 1214 | UAUCAUCCUUGUCAACUGUGCCACA |
| 1215 | UUAUCAUCCUUGUCAACUGUGCCAC |
| 1216 | UUUAUCAUCCUUGUCAACUGUGCCA |
| 1217 | AUUUAUCAUCCUUGUCAACUGUGCC |
| 1218 | GAUUUAUCAUCCUUGUCAACUGUGC |
| 1219 | UGAUUUAUCAUCCUUGUCAACUGUG |
| 1220 | UUGAUUUAUCAUCCUUGUCAACUGU |
| 1221 | AUUGAUUUAUCAUCCUUGUCAACUG |
| 1222 | UAUUGAUUUAUCAUCCUUGUCAACU |
| 1223 | UUAUUGAUUUAUCAUCCUUGUCAAC |
| 1224 | AUUAUUGAUUUAUCAUCCUUGUCAA |
| 1225 | CAUUAUUGAUUUAUCAUCCUUGUCA |
| 1226 | GCAUUAUUGAUUUAUCAUCCUUGUC |
| 1227 | UGCAUUAUUGAUUUAUCAUCCUUGU |
| 1228 | UUGCAUUAUUGAUUUAUCAUCCUUG |
| 1229 | CUUGCAUUAUUGAUUUAUCAUCCUU |
| 1230 | GCUUGCAUUAUUGAUUUAUCAUCCU |
| 1231 | AGCUUGCAUUAUUGAUUUAUCAUCC |
| 1232 | AAGCUUGCAUUAUUGAUUUAUCAUC |
| 1233 | UAAGCUUGCAUUAUUGAUUUAUCAU |
| 1234 | GUAAGCUUGCAUUAUUGAUUUAUCA |
| 1235 | AGUAAGCUUGCAUUAUUGAUUUAUC |
| 1236 | UAGUAAGCUUGCAUUAUUGAUUUAU |
| 1237 | AUAGUAAGCUUGCAUUAUUGAUUUA |
| 1238 | GAUAGUAAGCUUGCAUUAUUGAUUU |
| 1239 | UGAUAGUAAGCUUGCAUUAUUGAUU |
| 1240 | AUGAUAGUAAGCUUGCAUUAUUGAU |
| 1241 | AAUGAUAGUAAGCUUGCAUUAUUGA |
| 1242 | AAAUGAUAGUAAGCUUGCAUUAUUG |
| 1243 | UAAAUGAUAGUAAGCUUGCAUUAUU |
| 1244 | AUAAAUGAUAGUAAGCUUGCAUUAU |
| 1245 | CAUAAAUGAUAGUAAGCUUGCAUUA |
| 1246 | UCAUAAAUGAUAGUAAGCUUGCAUU |
| 1247 | UUCAUAAAUGAUAGUAAGCUUGCAU |
| 1248 | AUUCAUAAAUGAUAGUAAGCUUGCA |
| 1249 | UAUUCAUAAAUGAUAGUAAGCUUGC |
| 1250 | CUAUUCAUAAAUGAUAGUAAGCUUG |
| 1251 | GCUAUUCAUAAAUGAUAGUAAGCUU |
| 1252 | UGCUAUUCAUAAAUGAUAGUAAGCU |
| 1253 | UUGCUAUUCAUAAAUGAUAGUAAGC |
| 1254 | AUUGCUAUUCAUAAAUGAUAGUAAG |
| 1255 | UAUUGCUAUUCAUAAAUGAUAGUAA |
| 1256 | GUAUUGCUAUUCAUAAAUGAUAGUA |
| 1257 | AGUAUUGCUAUUCAUAAAUGAUAGU |
| 1258 | CAGUAUUGCUAUUCAUAAAUGAUAG |
| 1259 | UCAGUAUUGCUAUUCAUAAAUGAUA |
| 1260 | UUCAGUAUUGCUAUUCAUAAAUGAU |
| 1261 | CUUCAGUAUUGCUAUUCAUAAAUGA |
| 1262 | UCUUCAGUAUUGCUAUUCAUAAAUG |
| 1263 | UUCUUCAGUAUUGCUAUUCAUAAAU |
| 1264 | UUUCUUCAGUAUUGCUAUUCAUAAA |
| 1265 | AUUUCUUCAGUAUUGCUAUUCAUAA |
| 1266 | AAUUUCUUCAGUAUUGCUAUUCAUA |
| 1267 | UAAUUUCUUCAGUAUUGCUAUUCAU |
| 1268 | UUAAUUUCUUCAGUAUUGCUAUUCA |
| 1269 | UUUAAUUUCUUCAGUAUUGCUAUUC |
| 1270 | UUUUAAUUUCUUCAGUAUUGCUAUU |
| 1271 | GUUUUAAUUUCUUCAGUAUUGCUAU |
| 1272 | UGUUUUAAUUUCUUCAGUAUUGCUA |
| 1273 | UUGUUUUAAUUUCUUCAGUAUUGCU |
| 1274 | UUUGUUUUAAUUUCUUCAGUAUUGC |
| 1275 | UUUUGUUUUAAUUUCUUCAGUAUUG |
| 1276 | CUUUUGUUUUAAUUUCUUCAGUAUU |
| 1277 | UCUUUUGUUUUAAUUUCUUCAGUAU |
| 1278 | AUCUUUUGUUUUAAUUUCUUCAGUA |
| 1279 | AAUCUUUUGUUUUAAUUUCUUCAGU |
| 1280 | CAAUCUUUUGUUUUAAUUUCUUCAG |
| 1281 | GCAAUCUUUUGUUUUAAUUUCUUCA |
| 1282 | AGCAAUCUUUUGUUUUAAUUUCUUC |
| 1283 | CAGCAAUCUUUUGUUUUAAUUUCUU |
| 1284 | ACAGCAAUCUUUUGUUUUAAUUUCU |
| 1285 | GACAGCAAUCUUUUGUUUUAAUUUC |
| 1286 | AGACAGCAAUCUUUUGUUUUAAUUU |
| 1287 | GAGACAGCAAUCUUUUGUUUUAAUU |
| 1288 | UGAGACAGCAAUCUUUUGUUUUAAU |
| 1289 | UUGAGACAGCAAUCUUUUGUUUUAA |
| 1290 | AUUGAGACAGCAAUCUUUUGUUUUA |
| 1291 | UAUUGAGACAGCAAUCUUUUGUUUU |
| 1292 | AUAUUGAGACAGCAAUCUUUUGUUU |
| 1293 | UAUAUUGAGACAGCAAUCUUUUGUU |
| 1294 | AUAUAUUGAGACAGCAAUCUUUUGU |
| 1295 | GAUAUAUUGAGACAGCAAUCUUUUG |
| 1296 | AGAUAUAUUGAGACAGCAAUCUUUU |
| 1297 | AAGAUAUAUUGAGACAGCAAUCUUU |
| 1298 | UAAGAUAUAUUGAGACAGCAAUCUU |
| 1299 | AUAAGAUAUAUUGAGACAGCAAUCU |
| 1300 | UAUAAGAUAUAUUGAGACAGCAAUC |
| 1301 | AUAUAAGAUAUAUUGAGACAGCAAU |
| 1302 | AAUAUAAGAUAUAUUGAGACAGCAA |
| 1303 | AAAUAUAAGAUAUAUUGAGACAGCA |
| 1304 | UAAAUAUAAGAUAUAUUGAGACAGC |
| 1305 | AUAAAUAUAAGAUAUAUUGAGACAG |
| 1306 | AAUAAAUAUAAGAUAUAUUGAGACA |
| 1307 | UAAUAAAUAUAAGAUAUAUUGAGAC |
| 1308 | AUAAUAAAUAUAAGAUAUAUUGAGA |
| 1309 | AAUAAUAAAUAUAAGAUAUAUUGAG |
| 1310 | AAAUAAUAAAUAUAAGAUAUAUUGA |
| 1311 | UAAAUAAUAAAUAUAAGAUAUAUUG |
| 1312 | GUAAAUAAUAAAUAUAAGAUAUAUU |
| 1313 | GGUAAAUAAUAAAUAUAAGAUAUAU |
| 1314 | UGGUAAAUAAUAAAUAUAAGAUAUA |
| 1315 | UUGGUAAAUAAUAAAUAUAAGAUAU |
| 1316 | UUUGGUAAAUAAUAAAUAUAAGAUA |
| 1317 | AUUUGGUAAAUAAUAAAUAUAAGAU |
| 1318 | AAUUUGGUAAAUAAUAAAUAUAAGA |
| 1319 | UAAUUUGGUAAAUAAUAAAUAUAAG |
| 1320 | AUAAUUUGGUAAAUAAUAAAUAUAA |
| 1321 | AAUAAUUUGGUAAAUAAUAAAUAUA |
| 1322 | GAAUAAUUUGGUAAAUAAUAAAUAU |
| 1323 | AGAAUAAUUUGGUAAAUAAUAAAUA |
| 1324 | UAGAAUAAUUUGGUAAAUAAUAAAU |
| 1325 | UUAGAAUAAUUUGGUAAAUAAUAAA |
| 1326 | CUUAGAAUAAUUUGGUAAAUAAUAA |
| 1327 | UCUUAGAAUAAUUUGGUAAAUAAUA |
| 1328 | CUCUUAGAAUAAUUUGGUAAAUAAU |
| 1329 | ACUCUUAGAAUAAUUUGGUAAAUAA |
| 1330 | UACUCUUAGAAUAAUUUGGUAAAUA |
| 1331 | AUACUCUUAGAAUAAUUUGGUAAAU |
| 1332 | AAUACUCUUAGAAUAAUUUGGUAAA |
| 1333 | AAAUACUCUUAGAAUAAUUUGGUAA |
| 1334 | GAAAUACUCUUAGAAUAAUUUGGUA |
| 1335 | AGAAAUACUCUUAGAAUAAUUUGGU |
| 1336 | AAGAAAUACUCUUAGAAUAAUUUGG |
| 1337 | GAAGAAAUACUCUUAGAAUAAUUUG |
| 1338 | GGAAGAAAUACUCUUAGAAUAAUUU |

*At least one (i.e., one or more) nucleoside linkage of the oligonucleotide sequence is independently selected from a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

Table 3 below identifies exemplary STMN2 AON sequences:

TABLE 3
Exemplary STMN2 AON Sequences, in each one or
more spacers described in the present disclosure
are incorporated for generation of an
oligonucleotide of the present invention

| SEQ ID NO: | Oligonucleotide sequence |
|---|---|
| (legacy ID*) | (5′→3′) |
| SEQ ID NO: 31 | AATGTTAAGACATAATACCAGAGCT |
| SEQ ID NO: 36 | TTAAAAATGTTAAGACATAATACCA |
| SEQ ID NO: 41 | TAGATTTAAAAATGTTAAGACATAA |
| SEQ ID NO: 46 | TACCATAGATTTAAAAATGTTAAGA |
| SEQ ID NO: 55 | TGTAAAGATTACCATAGATTTAAAA |
| SEQ ID NO: 144 | AATCCAATTAAGAGAGAGTGATGGG |
| SEQ ID NO: 146 | AAAATCCAATTAAGAGAGAGTGATG |
| SEQ ID NO: 150 | TTTAAAAATCCAATTAAGAGAGAGT |
| SEQ ID NO: 169 | CCTGCAATATGAATATAATTTTAAA |
| SEQ ID NO: 170 | TCCTGCAATATGAATATAATTTTAA |
| SEQ ID NO: 171 | GTCCTGCAATATGAATATAATTTTA |
| SEQ ID NO: 172 | AGTCCTGCAATATGAATATAATTTT |
| SEQ ID NO: 173 | GAGTCCTGCAATATGAATATAATTT |
| SEQ ID NO: 177 | TGCCGAGTCCTGCAATATGAATATA |
| SEQ ID NO: 181 | CTTCTGCCGAGTCCTGCAATATGAA |
| SEQ ID NO: 185 | AGGTCTTCTGCCGAGTCCTGCAATA |
| SEQ ID NO: 197 | CCTTTCTCTCGAAGGTCTTCTGCCG |
| SEQ ID NO: 203 | TTTCTACCTTTCTCTCGAAGGTCTT |
| SEQ ID NO: 209 | TCTTATTTTCTACCTTTCTCTCGAA |
| SEQ ID NO: 215 | CCAAATTCTTATTTTCTACCTTTCT |
| SEQ ID NO: 237 | GCACACATGCTCACACAGAGAGCCA |
| SEQ ID NO: 244 | CACACACGCACACATGCTCACACAG |
| SEQ ID NO: 249 | TCTCGCACACACGCACACATGCTCA |
| SEQ ID NO: 252 | CTCTCTCGCACACACGCACACATGC |
| SEQ ID NO: 380 | TGTTTTAATTTCTTCAGTATTGCTA |
| SEQ ID NO: 385 | TCTTTTGTTTTAATTTCTTCAGTAT |
| SEQ ID NO: 390 | AGCAATCTTTTGTTTTAATTTCTTC |
| SEQ ID NO: 395 | GAGACAGCAATCTTTTGTTTTAATT |
| SEQ ID NO: 400 | ATATTGAGACAGCAATCTTTTGTTT |

*At least one (i.e., one or more) nucleoside linkage of the oligonucleotide sequence is independently selected from a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

In some embodiments, all internucleoside linkages of the STMN2 AON oligonucleotides listed in Table 3 are phosphorothioate linkages (except when a spacer is present, the linkage may or may not be a phosphorothioate linkage), and each of the linked nucleosides of the oligonucleotide are 2′-O-(2-methoxyethyl) (2′-MOE) nucleosides, and each “C” is replaced with a 5-MeC. In some embodiments, all internucleoside linkages of the STMN2 AON oligonucleotides listed in Table 3 are phosphorothioate linkages, and each of the linked nucleosides of the oligonucleotide are 2′-O-(2-methoxyethyl) (2′-MOE) nucleosides, and not all or none of the ‘C” is replaced with 5-MeC.

Table 4 below identifies additional exemplary STMN2 AON sequences: Table 4. Additional Exemplary STMN2 AON Sequences (corresponding to AONs shown in Table 3 but with thymine bases replaced with uracil bases)

TABLE 3

| | Oligonucleotide sequence |
|---|---|
| SEQ ID NO: | (5′→3′) |
| SEQ ID NO: 923 | AAUGUUAAGACAUAAUACCAGAGCU |
| SEQ ID NO: 928 | UUAAAAAUGUUAAGACAUAAUACCA |
| SEQ ID NO: 933 | UAGAUUUAAAAAUGUUAAGACAUAA |
| SEQ ID NO: 938 | UACCAUAGAUUUAAAAAUGUUAAGA |
| SEQ ID NO: 947 | UGUAAAGAUUACCAUAGAUUUAAAA |
| SEQ ID NO: 1036 | AAUCCAAUUAAGAGAGAGUGAUGGG |
| SEQ ID NO: 1038 | AAAAUCCAAUUAAGAGAGAGUGAUG |
| SEQ ID NO: 1042 | UUUAAAAAUCCAAUUAAGAGAGAGU |
| SEQ ID NO: 1061 | CCUGCAAUAUGAAUAUAAUUUUAAA |
| SEQ ID NO: 1062 | UCCUGCAAUAUGAAUAUAAUUUUAA |
| SEQ ID NO: 1063 | GUCCUGCAAUAUGAAUAUAAUUUUA |
| SEQ ID NO: 1064 | AGUCCUGCAAUAUGAAUAUAAUUUU |
| SEQ ID NO: 1065 | GAGUCCUGCAAUAUGAAUAUAAUUU |
| SEQ ID NO: 1077 | AGGUCUUCUGCCGAGUCCUGCAAUA |
| SEQ ID NO: 1089 | CCUUUCUCUCGAAGGUCUUCUGCCG |
| SEQ ID NO: 1095 | UUUCUACCUUUCUCUCGAAGGUCUU |
| SEQ ID NO: 1101 | UCUUAUUUUCUACCUUUCUCUCGAA |
| SEQ ID NO: 1107 | CCAAAUUCUUAUUUUCUACCUUUCU |
| SEQ ID NO: 1129 | GCACACAUGCUCACACAGAGAGCCA |
| SEQ ID NO: 1136 | CACACACGCACACAUGCUCACACAG |
| SEQ ID NO: 1141 | UCUCGCACACACGCACACAUGCUCA |
| SEQ ID NO: 1144 | CUCUCUCGCACACACGCACACAUGC |
| SEQ ID NO: 1272 | UGUUUUAAUUUCUUCAGUAUUGCUA |
| SEQ ID NO: 1277 | UCUUUUGUUUUAAUUUCUUCAGUAU |
| SEQ ID NO: 1282 | AGCAAUCUUUUGUUUUAAUUUCUUC |
| SEQ ID NO: 1287 | GAGACAGCAAUCUUUUGUUUUAAUU |
| SEQ ID NO: 1292 | AUAUUGAGACAGCAAUCUUUUGUUU |

STMN2 Transcript with a Cryptic Exon

In one embodiment, a STMN2 AON targets a region of a STMN2 transcript comprising a cryptic exon sequence, the STMN2 transcript comprising the sequence provided as SEQ ID NO: 1339.

(SEQ ID NO: 1339)
ACTTGTAATATACAGGTATCCCTCCTGGTAAGCTCTGGTATTATGTCTT
AACATTTTTAAATCTATGGTAATCTTTACAAAATATTTTACTTCCGAAC
TCATATACCTGGGGATTTTATTACTCTGGGAATTATGTGTTCTGCCCCA
TCACTCTCTCTTAATTGGATTTTTAAAATTATATTCATATTGCAGGACT
CGGCAGAAGACCTTCGAGAGAAAGGTAGAAAATAAGAATTTGGCTCTCT
GTGTGAGCATGTGTGCGTGTGTGCGAGAGAGAGAGACAGACAGCCTGCC
TAAGAAGAAATGAATGTGAATGCGGCTTGTGGCACAGTTGACAAGGATG
ATAAATCAATAATGCAAGCTTACTATCATTTATGAATAGCAATACTGAA
GAAATTAAAACAAAAGATTGCTGTCTCAATATATCTTATATTTATTATT
TACCAAATTATTCTAAGAGTATTTCTTCC

A cryptic exon sequence within the STMN2 transcript is provided as SEQ ID NO: 1340.

(SEQ ID NO: 1340)
GACTCGGCAGAAGACCTTCGAGAGAAAGGTAGAAAATAAGAATTTGGCT
CTCTGTGTGAGCATGTGTGCGTGTGTGCGAGAGAGAGAGACAGACAGCC
TGCCTAAGAAGAAATGAATGTGAATGCGGCTTGTGGCACAGTTGACAAG
GATGATAAATCAATAATGCAAGCTTACTATCATTTATGAATAGCAATAC
TGAAGAAATTAAAACAAAAGATTGCTGTCTC
(Source: NCBI Reference Sequence: NC_000008.11).

In various embodiments, the STMN2 transcript with a cryptic exon shares between 90-100% identity with SEQ ID NO: 1339. In various embodiments, the STMN2 transcript with a cryptic exon shares at least 90%, 91%, 92%, 93%, 94%, 95%, 96%, 97%, 98%, or 99% identity with SEQ ID NO: 1341.

In one embodiment, a STMN2 transcript with a cryptic exon can comprise a pre-miRNA STMN2 transcript. In one embodiment, a STMN2 transcript with a cryptic exon can comprise the sequence provided as SEQ ID NO: 1341.

(SEQ ID NO: 1341)
AGCTCCTAGGAAGCTTCAGGGCTTAAAGCTCCACTCTACTTGGACTGTACTATCAGG
CCCCCAAAATGGGGGGAGCCGACAGGGAAGGACTGATTTCCATTTCAAACTGCATTC
TGGTACTTTGTACTCCAGCACCATTGGCCGATCAATATTTAATGCTTGGAGATTCTGA
CTCTGCGGGAGTCATGTCAGGGGACCTTGGGAGCCAATCTGCTTGAGCTTCTGAGTG
ATAATTATTCATGGGCTCCTGCCTCTTGCTCTTTCTCTAGCACGGTCCCACTCTGCAG
ACTCAGTGCCTTATTCAGTCTTCTCTCTCGCTCTCTCCGCTGCTGTAGCCGGACCCTTT
GCCTTCGCCACTGCTCAGCGTCTGCACATCCCTACAATGGCTAAAACAGCAATGGTA
AGGCACTGCGCCTCGTTCTCCGTCGGCTCTACCTGGAGCCCACCTCTCACCTCCTCTC
TTGAGCTCTAGAAGCATTCAGAGATATTTTATAAAGAAAAAGATGTTAATGGTAACA
CAGGACCAGGAAGGACAGGGCAGTTCTGGGGGAGGTGGGAGGGCAGAGAAGAGGT
CTATGGAAATCTAAAGCGAAGAATTTCTTTTAAAAGGTAGAAGCGGGTAAGTTGCCC
TCCTATGGGTAGAGAATTTATTCTGTTTCCATATTTAAAATTAGGACTCAATCGTGAG
GGGAGGAAGCTACCTTAACTGTTTGCCTTAAATGGGCTTAAGGGACATTTTGGAAAG
TGCTTTATAACGACCTTTTTTTTTTTTATTTCTTCTCTAGTTTAAGAAGAAAATAGGAA
AGGGGTAAAGGGAAGGTGGGAGAAAGGAAAAAGAAAATTGCAAAGTCAAAGCGGT
CCCATCCCGCTGTTTGAAAGATGGGTGGAGACGGGGGGAGGGGATGGAGAGAACTG
GGCACATTTTACGGTATTGTCTCGTCGAAGAAACCGCTAGTCCTGGGGTGCGGTGCA
GGGAGGTAAGACGGCGGGGGACAGGGTGGGGGTAGGACCTCCGCTCCTTTGTTTTA
GGGCAAGGGAGGGGAAGGAGAGAGGAAGTCGCGGAGGGCGTGGAGGGCGCGGGTG
GGCAGCTGCAGGGGGGGGGAAGCGCGCGGCAGGGAGGGGTGGAGGGACAGCGGCT
TCGAAGGCGCTGGGGTGGGGTTTCTTTGTGTGCGGACCAGCGGTCCCGGGGGGAGG
CACCTGCAGCGCTGGGCGCACAATGCGGACAGCCCCACCCAGTGCGGAACCGCGCA
GCCCCGCCCCCCCGCCCGGTGCTGCATCTTCATTCGAAAGGGGGTCGGGTGGGGAGC
GCAGCGTGACACCCAGGAGCCCAACCCTGCGGGGACAGCGGCGCCACGCCCCGCGC
TCCCCGCTCCCGACTCCCCGCCGCGGCTTCCAAGAGAGACCTGACCACTGACCCCGC
CCTCCCCACGCTGGCCTCATTGTTCTGCTTTTAAGAGAGATGGGAAAAGTGGGTTAA
CATTTTTCTTTTCGGAAGCAAATTACATAGAGTGTTTAGACATAGACACAGATAAAG
GGTTCTTTGAAGACCTTTGATCGTTTGCGGGAAAAGCTTCTAGAACCTAGACATGTG
TATGTATAATAATAGAGATGACATGAAATCGTATATAAAGCAAAAGAGGTCAAAGT
CTTAAGTTAAGCCACGCGAAATTTCCGTTTTGTGGGTCAGACAGTGCCAAATATCGG
CAATTTCATAAGCTCAGAGAGACAAGACAGTGGAGACACAGGATGACCGGAAAAGA
TTCTGGATTCAGGGCCTTCATCCGCAATTGGTCTTGTGCCTTGAGTGCCCACGGTTCT
GGCGCTCAGTGGCCCCGGGGTGAAAAGGCAGGGTGGGGCCTGGGGTCCTGTGGCAG
CTGGAAGCACGTGTCCCCCGGGACTTGGTTGCAGGATGCGGAGACAGGGAAAGCTG
CCGAAAGGACTCCATCTGCGCGGCTCCGCCCTGCCCTACCCTCCCCGCGGAGCCGGG
GAGACCTCAGGCTCCGAGACTGGCGGGGAAGAGGAATATGGGAGGGGCAGTTGAGC
TGTATGCAGTCCTGGAACCTCTTTTTTCAGCCCCGCAGTCCACAACGGCCCGAGCAC
CCCTTGATGTGCGCAGACCCCCGGCGTGGCTCTCAGCCCCAGCACCGAGCCCCTCCC
AGCCAAGCGGGTGGCTCTGCAGAAAAGCTGGCTCGAGCCCCGCCCGGCCACACAAA
GGCGCGGCCCCACCCAGCCCGGGCGCGAGACCGCAGAGGTGACCCCCTTCCCAGGG
ATTCAGGGAGGGCTGTCTCTTCTCGCCCACCCACGGTCCGCGGAGCTCGGGGCTTTT
TTTCCCCCAGCCCAAGCCCCCCGCCCACCCTCTGTTCTCTATGATTTTCCAGAATGGA
GACCCCGCGAGGGGCTTCTCTAAGGGAGACCCTCGCTCCTCCAGCGGGGCGCGGCTC
GGCCCCACCCCTCCCAGCTGAGGCCCAGAGCCGCCTACCGCTGGCCGGGTGGGGGC
GCACGTGGCGACTGGGTGTGTGGAGCGCAGCCAGCCCTGCAGAGCCCCGCGCCGCG
CCCTGCGCTCCCCTCCCCGGAGTTGGGCGCTCGCCCCCGCGGTGCAGCCGGGGAGAC
CGGTTTCTGCGCAGTGTCCTGAGCTACCCCCGCTTTCCACAATTCGCAGTTCACTCGC
ACGTCCAGAAAGGTTCTGAGAATGGGTGGTGGGGGCGATCTCGCCTCGCTTTCTGCA
CCCCTCAGAAAGGTTTCCGCTGCAGGCTAGTGGCTGCAAACTCATCGTCATCATCAG
TATTATTATCATTTCAAATCGTTGTTATTATTTAATGATTCAGTAGCCTTGTTTGTTCT
CATTTGTTCAAAAGGGACGTGGATTGCTCTTGGTTAAGGATTAACCCTTGTTGCGTTC
GCTTTGCTTCCTCCTAATTGCCCTCATCCCTTTCCCCCACAAAAAGGTAAATTTGTCT
CCAGTTGTTCATTTTAAGTTATAAAGCAAATATATTTTTGCTTCCTGCCAGGATTATG
TATGTTCATGTGGCTAAGATACATGTGCAAGTGCTTGCTAAGAGCAGGGTTTGTGTG
CCAACGATTGCTGGAAAATTCTCTGCAAAGAATTGTTTGTGGCTGCAATGGGTGAGA
ATACACATATATAATTGAGATGATCTTCAACATAAGGTTATATCTATAAATATATAA
ATATAGTTTATGCACAAAATTTTAAGTTTTTTCCCCTGAAACTGTTCTTCCAACTGCT
GATTCTTGATACAGCCTCAATCCTACACAGATACATGGATCGTGAAATGGTAGCCGC
CATCCAAATAAAAATCCCACCCCAAATATGACAAACGCAAGCATCCTTTCTGGCCAT
AATTTAACTGCATTTGCAAATCATGAAAAAAACACTACTTCTGCAGTATTAAAATAA
TAGATTTTGAAATTAATTCCAATTTCAAAGATAATTAATTATCAGGGCGAGTGCTTTT
TTCCTGATTCATTAAACAATTATGTATTCAGCATGATTGTAAGAGGTGCATATAATAT
TCCCCATTATCTTTTCTAATGAAGTGGGCACCTTCTGAATGGATATATAAGTAACTAG
AAATGAAAAGCTGAGGATTTGGTCAGAATTTCAGGATAAAACTGAAAGAAATGGCA
GTAGTTTATCAATTAATCTCATGTATTTAGTTTATACCAGGTGAGTAAGCTGAGCCTG
CAATAAACACTCTCTGTCCCAGTGTAACACGTCGCAGGTAGCTAGAATGATAGGATA
AATTAATAGACCTTGTGGTGTTTGTCTATGCACGTTAAAATTCTCTGAGAGAAAGTA
TATTTTAAAATGATAATTAAGATTGGACATTTGTGCTATTAAAATCTACAACTTTAGT
CAAAATTCACAATGGTTTTTTTTTACAATAATGTGACTTACAGATTTGTAGTAAATTA
TTCTATTCTAAAAGAGAAATGAGTGTTTTTATTGTTACAGCTATTACCTCATTAATAT
TTTTAGCAAACTTTTATTTGTTGCATTGAAAGCAGTTTTAATTACTTTGGGTTTTTATT
TTTCAAATTACTAATGGATAGATGGTGGAATAAGCATTTAATCATTTGGCACAATAT
GACTTCCATCAAATAGCTCATTCTCAGTGATTAAAAAATGCTACAAGAGGCTACAAT
TTACTCAGATTCAGGAAATGTCCTTTCAGAGTGCCATAAGGCTGATTCATATAATAA
AATAGTTTTCTTCCCTATAATTTAAGATCAAATAGTTACTTAGTTCTGTGAATACCTA
GCAGTAGCTATCAAACAGAATTTTAAAGTTAAATCTGTACAACTAACAATGAAGTGG
AGGATGAATCGATACATATTGAATGGAAGACTTTGTCATTGATAAATTCAGGCCATC
TTTAGGAAAATTCCGGATTTATCAATCACCATTATTTTTTACTTCAACTGAGTGTGAC
TGATCACATGCTCAGGCTACCTTGGTAGCTCATTGCTCACAGGAGGCTGAAAAAAGC
TGGCCTCCGAGCAGGAGGAAGCTCAGAGCACAAACCTAGGCCTGGGCGTGGCCACT
GGGAGCTGCTGATAGCGAACCCCAGCTCACACCAGTTTCTTTTTTGGTCGTGGGAAG
AAAAACACATATTATCCTGTTGTCACAAGATCTGTGACCTTATATGAAAAAATGCTA
GAATTTTTTCATTAAAAAAGAAAATACTGAACTAGCCAGTGACCCAGATGTTTTCAG
AACCTAGACTGGTTCTGTCCATTGGAAAACCTCGGTGTCTGCATTAACTTTTCACCAC
ACTAGAGGGCAATCATGTTCTCTAAAAAAGCAGATGATTGATGTAAACCTAGTTCCA
AATATTAACTGTTTAATAAAATCTTTTCTTTTACCAGGAACATTCAAGTGTTTATTCA
ATAAGCTGATGCCATGCTTTACCCTAGTGGATGAACAGAGCTTGTACAATTTTCAAG
GAGACAGGATGAAATGAGTGGTCATAATCTGAAAGTAGATACACGCCCTGGTTAAT
TATTCCCTGATGGTTTTACTTCTCAGTTTTATTACATTGTTATTATAATACCATTTATG
TTACTTCTGAGATTTTGTAGTGGATAAATAGTAGAAAAATGTCAGTAGTAATAGCAA
AGTTATTTAGCAGCCGAATATTTTAATGCTTAAAAATAAAGGAATAAATTAAAGAAA
ATCATTGTTTACTTCTTCATCGATTGAAATGTGCCCCCTGTTCAGAGCACATCTGAAT
ATCAGAGTCTCCACCTGCAGAGAACATGCAGCTTAGCGAGTAAAACAGGCAGGTAT
GTGATACTGAGGAGGTGTACCAAAAACTGACTGCTGTTATTTTTCCCATCTTCTAAGT
CTGTCTTTCTTTTCCATTTAAAGATACCTTTTTAAATCTAATCCAATGTGATTTCAATC
TAGTTTTATCAGATTTCAACAATTATTGAGCATCTCCTTGTAGTGGTTTTCTGTTTATT
AGAAAATCGATGTTAATTTTAACGAAGTAAGAAGAAATATATAAGTATAAACTAATT
TTGGGTATCATCAAAAGTGGATTTTTTAAATATGCATTGATAGAATTATTTTTTGATT
ACATTTTATGTAATTCTAATCCAGCTATAAAATATTTAATAGTGTCATATTACTGTGT
TCCTCAAACTTTGATGTGCATATGAATTACCTTTGATTTTCATTAAAATGCAAATTCT
GATTCAATACATCTGGCTTGAGGCAGACATTCTGTCTTCCGAACAAGCTCCCAGATG
ATGCTGATTCTGACCACTAAACACATCAGTTTTAGGGATATTAACTTGTAATATACA
GGTATCCCTCCTGGTAAGCTCTGGTATTATGTCTTAACATTTTTAAATCTATGGTAAT
CTTTACAAAATATTTTACTTCCGAACTCATATACCTGGGGATTTTATTACTCTGGGAA
TTATGTGTTCTGCCCCATCACTCTCTCTTAATTGGATTTTTAAAATTATATTCATATTG
CAGGACTCGGCAGAAGACCTTCGAGAGAAAGGTAGAAAATAAGAATTTGGCTCTCT
GTGTGAGCATGTGTGCGTGTGTGCGAGAGAGAGAGACAGACAGCCTGCCTAAGAAG
AAATGAATGTGAATGCGGCTTGTGGCACAGTTGACAAGGATGATAAATCAATAATG
CAAGCTTACTATCATTTATGAATAGCAATACTGAAGAAATTAAAACAAAAGATTGCT
GTCTCAATATATCTTATATTTATTATTTACCAAATTATTCTAAGAGTATTTCTTCCTGA
ATACCATGTGAGAAAATTCTTAAGAATTTATTGAGTATGACTGTATATTTGAAAAGA
GTGTTTTCTTCTGCTTATCTAAGCCAATAAAGGATCTTCATTATTCAATTCTAACTTTC
TAAGGAAGTCAACCTACAGATCAGAAAGAGGATCTTCAAGGAATAGCATCAAAGAC
ATAGTCAGGTCTCCCATGCAGTGACTGGCTGACCATGCAGCCATTACCACCTTTCTG
GAAATATTATGCTGCAAAAATGATACAATACACGAAATATCTCAAATTAAAAAATAT
AACATTTCCCAAATAGGGCACTAAAAACATGATCCCAAATAAAACTAGCTTCAGGGT
TTGCAGAATATACTGTTACTCAACACAAAGTTGGACTAAGTCTCAAAGTTAGCCATT
CAGTTGTTGTTAACAGTTCATTTCAGGGTCTCTCAGAAGCTGGGAAACTTTCCATTTT
TGCAATTTCTTGTACATTGAAGGAAAGGAAGACACACTTAAGACAGCATTACAAAA
GTAATTCATGTTTTAAATGTTTAATTCTGGCAGTCGGGCAGGGCTCTCTGTATAACCT
CATTTGGAGATGACAAAAATCTAAACTTGAGGGCCTCGAGCCAATAAGTCTTCCTAT
TTCTTTACTCAAACATTTTCCCGCAATGGTGCTTTCTTTCAACTGTTTTTCTGGTGTAT
TCATAAATTCCAGATTCTCTATGGGAAGTAACTTTTATTGATTGATTTAACCCTTGTA
TAGCACATATAACATGCAAGGCATTGTTCTAAGAACTTTCCACATATTAACTGTGTT
AATCACTTAATAATCCTAAGTAGGTTCTATTACAGATATGGAAACTGAGGCACAGAA
AGTTGAAGTATCTTACTCAAGGTCACACAGTTAGTCAGATCCAGAATTTGGGCCCAG
GCCATCTGGCTTCGGAATCCATCTTTCACCGATTGCTGCTAGTCTCATATCTGTTCCA
TGTTAGAGGTGAGCTCCCATTGCAGAGGTCACACCTGTGATATCACCATTTTATTTAA
ACAGACCAGAGATGGTCTTCTCCTTTCTGATCACAGACTCACCTTGAAGAGAAAATA
CTTCCAAATTGATGCCTAGTTTTAATAGCTTACCTGGGGCTTATTCAAATAATTGCCA
TGATTTAGGCTTTGGGAGAAAGAGAGCTATGAGGCCGTGTGGGTTGTAACGTATGAG
ACACATGGCGTTCTGCAGGCTCAGCACAGCATCGATTTCTGGTGGGAACACACTCTG
ATGACCAGTTCCAGAAATAACATTGACTTAATCTCCTCAGTCCCATCATGGTTAGCA
CATTTCAAAATGCCTCCTTAACTACTTCCATAGGCCAGAGATATTTAGTTTTAACATT
TTGTTGAATAAAATAAATTTACACATTCACATTTAATATAACTATTAGATGTTATTTC
AAGATTCTCTTCATATTACCATCAAAGCAGGCAGGCAGGCAGGAGAGAACTGTAGG
AAGGTTTTGAATCCCTTGTGAAACATTTTTAATTATCTTTTAATAAAGGAATCAGGCC
CTGTCATTTGTCAAGGAGACATTTGCAGTAGTAAAGCTTGTGTTTATAATATCCATTT
TTATTAGTCATGATTAAAGATAACATTTGTGTACATTTGTTCTCACAAAACACTTTTA
TATGAGTGTAAAGGTTAATTAATGCATTTCAGCCATCATTTTGCTGGTCATGTGGAA
ATATAGCTTCTTTAGGAATTGTACTTAGAGTAGGAGCCACATATTATACTATAAAAC
CATAACAAAAATATTTTAAGTTTGTTCTCACTTGTTGTTGACCTCCAGAGTAAAATAT
TTAATACTCTGGAAAGTTATGGGTTTCAAAATTTATTTTATGGCAAGAAATAGATAA
TTACAGTTCTCATAGAGCACATTTAAAATAATTTATTTTTATAGGGCAAAAATATTGC
CTAGGACTGAATGATTTTTTTTTTTTTACAAAGATTGTAAAGCAACGCCTGCAAGAGT
GCCCATTTAGCAGTTATTCTTCTGGAATAATTGTATTTTGGATGTTGGAGTTCGCACA
TTAACCATTAGTACAAGTACCCAATATAACAATAGATCATCAGGATAATAAATCTGT
CCATCTTTTAGTTGTATGTCTTTATATCAGGATAAAGAGAATTGAGTGAAATTTATCT
AAACCTAGTCCCACAAATACTTTTACAAGAGAGCATGTTAAAGTGTAAATTAAATTT
TTATTAGCATTCTACTCTGTCTTTGGAAGTTTTTTTTCCTTATGAAATGCAGCCATAA
AGTTTAACTTCCATTAACAAAGCTGCTCACAGTAAACCTATTATAATAATAGTTTCCC
AGTTTGGGCTTCCTAGTGAGGAGCAACCTAACTCACACGAAACAACCCCAACTTATA
ATATATTGACTGTTACAAAACTGAGACCAGAAAATCCCATCAAGATGGTACTGTTAT
CATTTCCAGACTCTCGGGAAGAACATTAATCATCTCAGGCACTTTTAGGATAGACTT
ATTGCAGCCTCCCTGGGAACTCTGCTTCAGAACATAATTATTTTTATTAATGCAGAGT
TACTTTTTATTTCCAACAAAAATATCTATTGTTATTATTTAAGTCTTACAGCTTTATCT
GAGAAATTCCAATTAGCACCCTTCTCATAATAAATATTCAAACACATGAAAAATTAC
CAAAGTTGTTCTAGTCTTTTAATGACATATTACATGATCCTGCACTCTTGTCACTTTA
AAAATTATCTTTTTATTATATTTCTGATGATTTTTTTCTTATATAGTTTTTTAAAAGGA
GCAGGCAAGCATAGAAGACTAAAAAATGTTCAAAAGAAAAATTAAATCGCATGATC
TATCTATATGGGACCTTGTCATTTTTAGAAAACATTCACCTGCTTCATCCTTTTGAAT
CTTCATATAATCCCTCTGAGATGGGCATACTATACAAGTTGTCTTATTTAAAGATTGG
TAAATTTAAGCTCAAATAATTTATTCAGTGGCAAGCCTCAGAGGCAGACTCGGAACA
CAGGTCTAATATATATTATATATATATTATAACATATAATATATATATTACATATAAT
AAAGTTGTGTATATTATTTACCTATCAAAATATTTATATGTAATATATAAATATGTTA
TATATCATGTATGTGCCTATTTCATACATATATACACATTCATGCAAAATAAGGTTTA
GCACTCCCTCCACTGTCCTGTAATAAAACATGCACAGTGAGAATAGTCATACACGAG
GCATATTTGTCTTCAGTTTAAAGTCATTGATAGTCAGTGTCACTAACTAAAGTAAAAT
AGATTGGAGCACCAACTTTGTTCTGAAGCCTGTGCCAGGTATTATGAGAACAAAAAT
AAAAATGTTCCTCACCCTTGGTGGATTTAGTCTTTTGCAGAAAAAAAGATCCTGTAC
ATGTCAGAAAGTTCAATAGTAATAATGGTAATTTATAACTATAAATGGAAGTCACCA
TCTCACAATTTCACCATCTTAACAATTTTGTTAAACTGCCCTACAATATTACAAGATA
GTACATAATGATACACTAGTAACATCAACTAGGAAGTACCAAGATCCACCAAAAGG
CTGAAAAATTTAAATATTTAATGAGTCCATCAACCAATCTGGCCAGAGAATTCTTTA
ATTAAAATGCTTCCCAAATTTTACTGAGAATCAGCAGCGTTTGAGGAGCTAGCCTCC
ACCCCCAGAGGTTCTCACTCTATTAGGTCTGAAGCAGGTCCCATGGATTTGCATTTCT
AACAAGCTCCCAGGTGGTGCTGATGAGGCTGATTCAGAACCACACTTGGAGTAGAC
CTAAAACAGCAGTGACCTGTAGGGTCCCCAAGCAGCAGGCCAGGACAGCATGTGAG
TTACGTCCTCTGTGGAGCTCTGCAACAAGGCGTCAAGAGGTCAGAGTCTAAGTCCCC
ATCAGCTCTGCCCTTCTCCACCAGTGCTGCTGGTGCTGCATGGAAGGAAGAGCCCAG
AAGGGATTCTGAGTTTCAGTCTTTACTCTTGCTGACGCACCTTGGTCAGGTCAATTTT
CCTGTTTGTTCCTCTAATTCAGCATCTGTAAAATAGCCATGTGAACTGCCTTGTCCAT
ATCAGAGGGTCTTTTTCAGACTCAAGGAAAAAAACGTGAAAGTGATTAGTGTCTGTC
AAGTAGTATATAAATGCAAGAAGTTGAGTTTTTAAATTGTCATTAGATATAAATACC
CATGTGCATGCATTTAGAATGAGTAAAGAGGGAACAAGGAGCGCAATCAAAAACTG
CGTCATTTGCTTTTTGAAAAATACTTTCTATGTAATGAAAAGTGAAATAAAATGTTA
ATTGAGTCCCTCTGACAACAGCATCAGACGTTTTGCAGTTCTTGTGATTAGAACCCA
CCTGGCCAGCCCTTCTTCCTCCTAAAGAAGAGCCTTCTTCTTCTTAAATGAAGGTTGG
CTCAGAAGAAGCAATTAACTCATTCAACGTTTTGTTACAGTCAATCCACATCCAACT
TTTCCCCAACTCAATCTGCTTTAAGGGAAGGATGGTAAGTGGTGGCCCAAGATGGCA
ACCATCAAGCTTAGAGAATCTCTAGAAGCAGGGGTGTCCCCAGCAAGTAGACACTG
AAAATATGAGAGGGCTGATAAGCCAGAGATAAAACTCAGTACTTACTTTGCTTCTAG
TCCATGTCTACCCCTTTCTTGGCACCACCTTGACACTACCCTCTGAGTCCACCTTCCT
GAGATGGTACAAACTCTGCTTAGACAAAGCAGCCCATGTCCAAAGGTGTTAGGGCTC
AGTTTAAAGCTGCCTTCAAAAGTTAAAACAGAAGTGTAAAGTTCTGTGCAATTAAAA
ATAATCAGCTTGTCTTGGAACTCAAACGAATGTAAAATCCTATGAAAATTAAAAAGC
AGTACCACAAGTTACCCCAAAAGTCCTTAGGTCAGTAACTGTTCCTGTTACAGGTAA
GAGAGAGCATGGATTAGAGGTGGGCGTGGGTATCCAGTGGACATGGTTTTGAACCA
TGCTCCACTACTACTCACTATCTGAGAATTCTTAAATTTATTAATCATTTCTATATTAT
AATTTTCTCAGTTATGAAATGGGAAAACAATACCTAAATCACATGGTTGTTAAGTAA
GCAATTGATTGTTAAGCATTTGGTCATCAAAAATATTAATCCCCTTCCCTGATTCCCT
AGATAAATGATGAAAATACTAAATAAAAATAATAAAAATTTAAAGTGAACATCTCA
ATTCTTATACTTTGTTAATTTCTACATGTATTACAAATCTACTAGAAATTACTTGGAA
TTGAGGAAATGATTACTGCTTAATAATTCTTTGTGGTAGAGGGAGAGTTGGTATCAT
ATTTATGAGACAGCAGCCAATATAGTATATCTCAAAGGAAAAAATCCATTCTACATA
ATGCCAGAATTTAATAGTTAAGCATTTTATCTAGGTCACAGCACAATAAGCAAGATG
GATAATTAAAATAAAAGTATATTTCTCTTGCATATATTTCTCATTTCATGTTTCCCTAT
CATATTTTATATCTTACCTTACTTCAAATACATATATACCTTCAATAAAACTGAGCCT
TCTTGCTTACCCAGGAAGTTTCATCATTCAGTAGAAATAAAAGATGACTTTAGAAAT
ATTAAAATACAAAAATCTACACTGAGGTCTTTTGAATGCAGGAAAAAGAATTATATC
ACACACACACGTACACGCACGCATGCATACACACACACAGAACCTCTCGTTCTTTCT
TAACATCTTATCAATCCATCAGTTTCACTCCCACTCCGTATCACCTGACTGTGCACAA
TATCTCATTGCCACCTCCCAGTCTTCTCCCTGCCTGGCACCCTCCTGCTCTCCTGCTTC
CACTTTAAACACCCTTCCTTCAGCTAGGTCTTTTCTTTCAGGGATCCTCCCGTTGCTTT
CTTATCTGGATCAATTTAGCCTTCCTCTTCTCCACCCATTAGTGGATAAGCACGACAA
AGACACTAGAGTCAAATAATACAAACAGAATATACCTTAGATGAGTATGGTGATGA
AAAGGATATGGATACTTAGAGTTTAGCACTATTCTCTCAGCCACTCAGGAAAGCAAC
GCCTTTACAATCAATAGTGTTTCAGGTACCAATCAATAATCTGTTATTGCTATTTTTA
AAATCTATAAGGTATCAGTAAAATGTAATTACTAGAGCAACAAAGATATCTTGTGAA
ATCAAATTAGTATTCATCCAGCAACTGAGTACAAAGGTTTAAGGGAGGATAACTACC
AATACCAAAACATTTTAAGCATTTTGTTTTGCCTCCTAAATATCAAATCATGTAAATG
TGTGGTACATAAATTAGGAATTATATTTATGACATAGCTGCAGACATATTAAGAGAA
ATATGTGCTTATATTTACAAGTATAGTACAGTTCTTTTTCATATTAGATACTGTTGAT
GATAATCTGCATATAAAAATGCTCAATATTTTTTCACATTTATAAGCCATAAAATAC
AGCTAATAAAATGTGTTTCTACTTTCTCATAAACATGGAATAGTGACAAACAAGGAG
CTTTATATGAAAGCACCATTACAATTTAAACTCTCACAAGGTCATAATATATTGCACT
AAGCAGGAGAGTTCAGCTTATTTAAAAAAAAAAATAAACTCTAATGAGGTTCTGGA
ATGCAGAGCCAAAGCATAAAGATGGAAATAAAAGAATTGCATGTCTTCTGAACTGA
CTTGGTTGATGATTTTTTTAAAAAAGGTTTTGTGTCTTCTGACTTGGTTGATGATTTTT
TAAAAAAACGTTTTGTGGTAGAACAAATAAGGTAAATGAAATTCAGTATTTAGGATG
AAAAGTTTTTCTAATTTCAGGAACAACATTGAAGAAATATTGAACTAAGCAGCTTTG
AAAGAATCAGATTCCATTTGTTGAAATTTTTCTGAGAATGAATTTTTTTAAGACAGTG
TACACAGTTGCAGTGTGTATTGGTTATGGATTGTGGCAAGCTATATTACAACTTACCC
AAGAAATAAGGAGGCTGGGCGTGGTGGCTCACACCTGTAATCCCAGCACTTTGGGT
GGCCGAGGCGGGCGGATCACGAGGTCAGGAGATCGAGACCATCCTGGCTAACACGG
TGAAACCCCGTCTCTACTAAAAGTACAAAAAATTAGCCGGGTGTGGTGGCGGGTGCC
TGTAGTCCCAGCTACTCGGGAGGCTGAGGCAGGAGAATGGCGTGAATCCGGGAGGG
GGAGTTTGCAGTGAGCCGAGATTGTACCACTGCACTCCAGCCTGGGCGACAGAGCG
AGACTCCGTCTCAAAAAAAAAAAAAAAAAAAAAAAAAGAAAGAAAGAAAGAAGGA
AAAAAGTCACTTGAAAAGAATACTGGACTTTGTGTCCAGCTTGCATAGCTGAAAAGA
ATAAAAACCTGTCCACTTAAACTCATTGCAAAAAGAAGATGTCACTCCTACAAATAG
CAAAGAGTCATGAAATTATTCTATCCAGAAAAGTATACATTTCATCCCTTTGGATAA
ATTTTAGAAGTGAACTATGAATACATACGGTGAGGATAGCCAGCTAAGAAGTCAAG
AAGGATTTCTCAAATTTGCTGCTCAGAAAGATCATACTCTCCACAAAACAAATAATA
GCAGGCTTTCCAAGTCAACCTTGAATCCAGCTTTCCTTTATCTTTCCTTCTTGTGAACT
TTCACTAGTTTACTATCTAACAATGAATTTGACGATAGCCACATACCATCTTATAGCA
ATATTTGTTATCATATCCCTTGTTATTTATCATTCACCTGCTCTGCTTGAGCCAGCTAC
AAGTCACATGTCCCACGCACTTTTTCCTGTTTGATTTTTTACAGCACTTTGAGACATG
TCTCATTATTCCTACTTGACAGGAAAGAAGCCATGGAAAGTTGAGTGACTTGCTCCT
GATCACAAATGCTGGCCAAGGAAGAGTCGAGTTTCAAATCTAATGATCTTTCCACTG
CACTCTAGATTCCTCATTTTGAACTATTTTTTTATTTTTTGCACTATAGACTTTTTTCC
ACATTTTGAACTGTTTTTTATTTTTTGCACTATAGACTTTTCTCTTATACCCAACTATA
TTGATGACTTCTTTTAGGCTAGAAACTTGTTTCACTTACTTTCCCTTTCTTCAGATTGC
TGCAATATTGGCCAACATGTATTGGGTACTTACTGAGTCAAGTACTGTGATTGTGCC
AAGTATCTTATAGGAGGATTATCATCCTCATTTTTACAGGTGAGAAAGGAAAGGAGG
TAAAGTCACACACAGCCAACAAAAATGGTAGCACCAGGATTTGAAACAAATCAGTC
TGACCCAAGTTGACTTTGTTAACCACTGTATGCACAGTCTTCTTAGACATAGTAAGA
GCTCTAATTGTGTTTGGTGATTTGATTATTATGACAAAGTAAGTAAGGGAAGCAGGG
AGAATTATAAGAAATAAGGCTCCACAACACTTGGCTATAGCAAAGCCCCTTAAAACT
TCAAAAGGTCACCCAAAGAATAAAGATCAGGCTGGGAGCAGTGGCTCACGCCTGTA
ATCCCAGCACTTTGGGAGGCCGAGGTGGGTGGATCACCTGAGTTCAGGAGTTCGAG
ACCAGCCTGGACAACATGGTGAAACCCTGTCTCTACTAAAAATACAAAAATTAGCTG
GATGTGGTGGTTGCCGCCTGTAATCCCAGCTACTTGGGAGGCTGAGGCAGGGAGAAT
CGCTTGAACCCAGGAGGTGGAGGTTGCAGTGAGCCGAGATCATGCCACTGCACTCC
AGCCTGGGCAACAAGAGCAAAAAACTCTGACTCAAAAAAATAAATAAATCAATCAA
TAAAATAAAGATCAATTTGGAGAAATTAATGCTTATTAATAAGCAATGTCTTGCACA
GCACTTCAGTTTCTCAATACATTACCTAACTCAATCCTTACAACAACACCCTATCCCC
ATTTTGTGGATAAATAAACTCATGTTCAGAAGGTTGAATAAATTATCTAAGGTTAAT
AGTTCCTGACCTAGAGCTCAAATCTTCAGTTTCTATCATATTCTTGCCCTTACCCTGG
GGTAGCTAACATTCACTCACTAGTATTGGAGCTAAAATAAGGGAGAGAACATATAA
ATGAATACAAAGGAGACATTCACCTGCCTTCTCTTTCTCCTTACATAGAGAAGGTTG
ATTATCTGCTATTGTGAAGTTTGCCTTTTGAAGGATAGAAATGAGAAGACTTTCTTAA
ATTTTGCCTCTACGCCAAGAAATTAGAGTGGTACCACCAGTAGTTCCATTTTCAAACT
ATCACTGTAGCTAAAGCTATGTGGTAAGGGCCAAGGAAAAGAAGTATTCTTGCACTT
CAAAATGCACTGAAATACCAGTCAGTAGCATAATATAAAGGAATTTAGTGGAGAGA
AGAGTTGACCTCAATCTGGCTCCAACATCTCGGCTCTTAACCCCTACCCTACACTTGT
TCTTCATGGGGAAGCTAATTGGGCCACTGGAAGATTCAGCAGCTACCATTTGCAGCT
GAGGGACAGCCCCTCCCTGCTTAGCAACCAATGGATATGCATTTATGGAACACCTGC
TAACTGCGACACACACTCCTATGTATGAGGGAAAATACAAAAAATGTTAAAGGAGA
TGCCTTCCCTTGCCCTCAGGAAACTTAAGTATAGTTGCAAAGAAATGATTAGCAGCA
AACGAAACCATGGAGAAGTAAGGGCTAAGGTCTGTGAAACAAGCCTAGAAAATAAC
CTTGTCCTTGAAAAACACAAAAAGAAAGAAAGAAAGAAAAGAAACTCCAAGGCCCT
TGTGAAGGAAACCATTAAGTTTGCTTCACTTCTGTGTTTAGGAAGACACAAACCCAG
TCTTAATGAACCTCAAGGCCACAACTACTGGAGACATTTAGGAATTGTCACCACATT
CTAATGTATATATCCTCTGTTTGGCCCTTCCTATTAATATTTTGTAAAATTTTTGAAGA
TATGAGCAATGTTTAAAACCATGAATCCCCCTTTTTTTATAAGTAATATTTAGGCTGA
ATAAACAAGAGAAAATAGGACATAAAGGGGAGCCAACGTGTGCCTTCATTTATAAT
GTATTCCCAAGTTGTGAGTTTGGTTTATCAGCAATTTATCATGCCAAATTCCAAGTCA
TATTTATCTATGCAGATCAAACACTTGATTCTATTTTTGCCTTAATTTTTTTATTGGGT
ATGTTTATGACCAAGTCATATGGTATTTTCTGTGACAGATAAAATGCACAGGTTATTC
CAATCTGGCTCAGCCAGTCATAGCAACATGTAGTCCTTCTCATGTCTTAAGAATGAG
TATCAAGAATTCAAAGGGAGTTCCAGATGGCATCCAAAAAGCTTACAGTTTATGCAT
CACTTATTCTAACAGTAGAAAAAGAATATTTGAAGCCAAAAATAGACCTTGCATGTA
GCATGTGGAAGAGTAGAAATTGCCCTGATAGTTAAACAATTTGAAATTCAAGACATT
AATTTCTTTATGAAGCATTTGTCACATCATAGGTAATATTTTATGCCTATCATATATA
TACTTATTATGAAATACAAAGAAATTATTCATTCTATCTAAGACTTTGTATCCTTTAC
CAATATCTCTCCATTCTCCCACCTCCACCCTAGCCCCTGGAAACCACCCTTCTACTCT
CTGCTTCTATGAGTTCTTTTTTAGTGAGATCATGCAGTATTTGTCTTTCTGTTCCTGTC
TTATTTCACTTGACATAATGTCCTTCAGGCTTATCCATGTTGTCACAAATGACAGAAT
TTCCTTCTTAAGGCTGAATAGTATTCCATTGTGTGTATGTAGCACATTTTCTTTATTAA
TTCATTTGTTGATGGATACTCATATTGATTCCATATCTTGGGTCTTGTGAATAATGAT
GCAGTGAACATAGGAGTGCAGATATCTTTTTGACATACTGATTCCACTTTGATGGGA
TATATACCCAGTAGTGGGACTGCTGGATCATCTAGTAGTTTTATTTTTTTTTATTTTTT
ATTTTTTTTATTTTGAGACAGAGCCTTGCTATGTCGCCCAGGCTGGAGTACAGTGGTG
CCATCTAGGCTCACTGCAATCTCTGCCTCCTGGGTTCAAGCAATTTTCCTGCCTCAGC
CTCCTGAGTAGCTGGGATTACAGGCACGCACCACCATGCCCGGCTAATTTTTGTATG
TTTAGTAGAGACGGGGTTTCACCATGTCTCGAACTCCTGTCTTCAAGTGATCCGTCCA
CCTCAGACTCCCAAAGTGCTGCGATTACAGGTGTGAGCCACCACGCCTGGCCTAGTA
GTTCTGTTTTTAATTTTTTGAGGAGCCTCCATACTGCTTTCCATAATGGCTCTAGGAA
TTTACATTCCACCAGCAGTGCACAAGGATTGCTTTTCTCCACATTCTGGCTAACCAGT
CTCCTGTCTTTTTGAGAACAGACATTTCAACACGTGTGAGATAATATCTCATTGTGGT
TTTGATTTGCATTTCCCTGATGATTAGTGATCTTGTGCCTTTTTTCATATAACTGCTGG
ACATTAATATGCCTTCCTTTGAGAACTGTGTATACAGGAGAAAATAATCACTTCTCA
GAGGAGCTTTCATTTCAAAATATCCGGGAAAAAAATAGAAAAAATGGAAAATTTAT
CCTAGAGTAAGTTGTCTTTTATATTTTGACCCTGTTTGTGACATAAACTGGATGATAC
AAAACTGGAATGCAAAGGCTTTAGGAGGATTACTTACTTACTTGTATATTGCTTTAG
GTTGTTTGCAGAAAATTATACTAATTGAAGTTCAGGCTATGATGTGATAAAATCTAT
GTCAGGAGATGAGTCTACATGCAAAGTTTGAGGAAGTGACATTTGAGTTTCAAAACA
AAAAAGCAATTTTCAATGTCATATCTAGGTTAACCCAAAAGATTTCTTTCACCCTATT
TAGCTGCCTCTAAGATGGATGCTGAGGATAATTACACTGTAGAACAATAGGACGATG
CTTCACACTCACCTCACAGGCTCTGTTATTCCCACATACTGCCAGAGATACTCCAAA
ATAAAATCACTGCAACATCAGGCAGTTATAAACCTCAACGGTATTATTTTCTATTTAT
ATACAGTATATTTTATATTTTACAAGTATAAAATAGAATATATTTATTCTATTCTCTTT
GACACAAAGTGACCATAAGACATATTACTTAAGTATGACTAGCAAAGTCATGGGGC
TTGTCATTCAGGAGGAAACTCTTAACTAACTGTTCAGTTTTTGTTCACTGCACCATTT
ACATAAGCCAAACTAATGCTTCACACTGTGCAAAACAATGCACAGTGTTGTGAATGA
ATGGCTAAAATAAAACTCTAATGAGTGGGGTTTGAAAAATGCAACTTTAGAAAACT
GTTGAGAAAATGTTGCACACTGCGCATTTTACAAAATTTCGTTGAAGGACACTGGAT
ATTCTTTTTAGGATTATGGAGGGAAGCAAAATTTTGGCTCCTACATGCAGTTTTTGTG
GCCTTTGCCTGAAATAGTCATCTCCCATTAATTATTTAGATATCATTCATTTCCTAAG
ACAACATTTAGGGAGACTGCCTTAAGTACAATTTGTACACTACCCAGATAAGAATTC
TTTTTGGTGAAACATCGATAAATATTACTTGGCAGTAACACCAAGTTAAAATATTTG
TTTCACAGTCGACGTTAATAACTATTATAGATAAAGTGAATTTTATAAGACATACTC
AGATCTAAAACAGCAATATGGAGCTCTTCAAATCCATTGAAACTTCATACCAGCCTA
CGGAAGTAGAGGTTTTTATGCAAACTCTTCAAGAAATATGCTCTGAACTTTTAATTCC
TTAGATTGATAGAGGAATTAAATCATGATATAACTAATAGGTTTGTGGTACAAATTG
CTGCTGCTTAATCTGACTCTGTGTCTTCCCAGTGTTCTATATGAATTAGATATTCCATT
ATCTAAAGACAATCAACCCCATCCCACGGTGATAGCTCTAGGACTCCCTTTGAGTTC
ATTAAATCTGTATTCTCAGTCTCCAAACTTCTGGTTAATTCAAACAGAAAAGTCAACT
GGCCCATGAACTAAAATAAAGTCATCTGAATTTTTTTTTTATTTTGCAGTGTGATAAA
AGTCTCGCACTTTTTATTTCTGAAAGTTTCTGCTTTCACTGAGAGCATAATAGGCTAT
CCACCCTTATGCAATCTTACATACAAAGTCATAGTCAGGCTAAATTCAAAAACACAT
GTGAGATAGAAGTCAACGTTTATTTTCTGGAGAAAAGCCACACATTACAACAAAGTG
AACAATGAAGCTGGCATCCTTATCACTGGTGACCAAAACATTTGTGACTCTGGACAT
TGGCCCCACAAATGCGATAAACATTCTGCATAGGAAGTGAGTTTTGCTAATTAAAAA
TGGATCCAAAATACTTTCTACTCTTCAGCCAAGAATTAAAAAGTAATAGGGAGGAAT
TGAAATCACTTGGGTGCTACATTGAGCCATTCTGGAGAAGCAATTCAGAGAATGTCA
TGGCAGCCTCAAATTGCTGCTCAGGAGCATCCCAGCTTAGAAGATTGCAGGAAAGG
AAGAGCAAAGTCATTCTTACATGAGAACTGTCCTTAACCAGATGAATAGACTCTCCA
TTTTTTACCCTGGCTTTGTCTCATTTAAGTCCCAACCAATCTAGCTATCATTTTAGGTT
TTACTACCTGCTAGTATTTAGGAGCTTAGGGGGATAAAAAAATCCCTCAATACTCAG
AATTAGACTTGGTGATAAAAATCTTGACACATAAACAGAATAAAGCGCTTTCATTAC
TCCTCTAAACCACAGTGTCATTTGGTCTCTATCAAGGACTGTAAGAATTTCTTTCATC
AGGGGAAAGAAAAAAAGGACAAGAGCCTGCAAGATGTAGCGGAACTCTCATTAAA
CACAGCAGGAGCTTTAACTGGAATCCAGAGTAAGGTGAGGTACCAGGTTACAACAA
TTTACTGCTTTTATTACAATTTTGATCACAAGGACTGATTCATGTCATCTAGTTTCTTT
TCCTTGTCACTATCACTGGTGCTAAGAATACATCAAATTGAAATTTAAGAGCCTCAT
ATGTTTCTGTATAACCCAGTGATGGGTTGTACTGCTTTGACCTTCTTAAATGTCCCTT
TATTTCATTTGATATCCATTCCCATAGAAAAACTATAATGCTTTGGTTGGTCAAAATA
TTAATCTTTCAAAACCTCCCTGGCTTAGAAAACCAAATTTTTGTAGAGAGAGATGGG
TAGAATCTAATTTTATTCTAAAGCAATTAGCATTACATCATCACAGCAGAAATATCT
AGAATATTACCTCATGTCAGTGATCTTCTGATATGTTAAAAAGGGTATTTTAAAATCT
GAGTTATTTCTTTTTCTTTTTAAAGTTACATCATTAATTACATACTCATCAACCAAAA
TATTTTATGCTCCAAATTTGAACCGATATAGTATGTAAGAAGTGTTCAAAATGAAAT
TATTTTGGTCTATTTTGTCTTTGAAGAAGATCACAGGGATGGACCTCCCAAAAGGAT
TTTTAAATGGGATTACATATCTGACTTTTAAAAAAAATTATCTGACCTTGAGTTATAG
TGCCCCAAAGTAAGCAAAGTTCCAAACACACAGTATCATCAGAATTGAGTTAAAATT
ATCACCAGGGGCTTAATTTCTGAAATTAAAAAGGAAATGTTATTTCCTTATGAAAAG
AAAAGGAACCAAAAATGAACTTCAAGGTAGCTGATTTCTGTCTATGTTAAGACTTAG
GTAATGGGAGAAAGGGAAAAGGAAGGACAGAATTAGGAGAGGAGCAGTGTTTAAC
AATTGCGGGTGCAAGACTCAAGTTTTTTAGAATCCATTAGCAGAGAACCCTATTTCT
CCCATTAACTGCTGTCCTTTTAAATCCTGGCACCAGCTCTGAGGACTGCAGGGTCCAT
AGCTAGTGCCCCACTCTACCCAGTTTAAAGACACCACTGCCTGGAAATGACAGGGGT
TTTTTTCTTAAGGAAAGAGGTGCTTTCTGCCACGTATATATAAATTGGTAAGCTTCAA
ATAAAGTGCTTTTGTCCTTTCTGTCTATCAGAAACTGTGCAAATCGAATTGCTGTAAA
ACCAAGGGCAAGAGACATCAATCCTGCATTCTATAGCATCTGATTTTATCCTTTATCC
CCAGGCACATTTCAAAAGGAAAAAAATGAGGTTGCATTTAAATTGAGTATTTGGGAC
TTGCCAGGAAAACCTCCCGCTAGACTAATATGATTGCAGGGAAAACAAGAGAAAGG
AAAAGTGGAGAGGGAGTGTGCTAACAGATCCTGGGCCTCGTCAGCAGAGCCGTCCT
GAGCACAAGGCCATGGTCAGACATCTGGTCCCGCGAATGACGTTTTCTTTATGGTCA
TTAAGAACACCAGTGTGTCGGGACACAAACAAGTATTCCTTTCAGGGATTATGACAC
ATTTTCTCCCAAAGTAGTATATTAATGACATTTCCAGAGCATTCTTTACTATCTTTTAT
ATGTGATCAGGAAGACTAATACATATCACTACTTCTTTTACACACAGCATTAGCCAA
AACTAAAGTGTCAAATACAATTTTGCCTAGGATGAATAAACAGAAGAAATTTTTATG
ATACTGCACTATCAATTCCAAATTAAATAACAACAAAATGATAAGTGTTAAAATTCA
TATTAATGATTGTTCCCACACAAGCCGGAAAAAATCTTTCTAAGAAGTCTTTCATGA
GTTAATCCCATCTTTCAAAGTGTTCAGTGGCTCCGAATTCAGTTACTGTTTCCTATCA
GTTCTTCTTTCATTAAGTCTCTTCCCTTTTTTTTCTCTTTGCACTATTTCCCTTAGCCGG
GTACATAATCTGCTGTGCTTTATTCATTTGTGTCTTAAGTTTGTTTCCCGATGACATAC
CTTTCCAGCAACGCCATCTGGGGAGTTTGGGCAACTGTACCACGTTAGGAGGAAACC
CTTCTTCACAGGAGAGTGTGCCTTTGCTGCAGGGAAGGAATTAGGATTTGCTTGGAC
TGTGGTTGCAGCTGGCTTTTAAGGATCTCCTTAGAATGCAAGCAACTCATCAATGAG
AATCTCTGCAATGGTTGTCACTGGGTAGAGTCATGCTATGTGGGGTCATAGCCTTTG
AAACAAATAACAGTAAAGATAAAAATGCTATTAAAGGAATCACCACCCACAGAGGT
TAACTGGGTTTTGTCCCCAGACCACCTCGAACAAGAAAGAACATTTTTATCAGTCAT
TTTCTTAGTTTTAGCTGATAAAACAAAGTACCATAGACTAGGTGGCTTATAAACAAC
AGAAATTTATTTTTCACAGCTTTGGAAACTGGAAGTCTGAGATCAGGCCGCCAGAAT
GATCAGATTCTAGTTAGGGCCTACTTTGCTTTTGCAGACTGCCAACTTCTAGCTGCAT
TTTCATGTGGCAAAAGGAGATTGAGCTAGCTCTCTGGTCTCTTCTTATAAGGACACT
AATCCCATTCATGAAGGCTTCACCTTCATCATCTAATTACTCTCCAAAGACCCCACCT
CCAAATACTATCACATTGGGAATTAGATTTCAAATACAAATTTTGCGGGGACACAAA
TATTCAGTCCATAATAGTAATGATTACTCATTATACATAGGGCTCTAAATGTGCTAGC
TTCTGATAGTTTTTACACTCACTTCTCTTTATTAGCTTGTCAAGCATAATTAGGGCAG
TGGCCTTACTGAAAATTATTGAATTTAGTTTCCTAAGGACAGATATTGAGGAGTTTTT
TCTTCACTAAAAATTCACGTTCCGATACAGCTTTCATCTGTTACTACTTTGTGAGATG
GAAAATCTTTTATTTTATTTTTATGTTTGGATTGACCCTTCTTAATAAAGTCGGCATGT
AATATGCTTCATGTGTTTCTAATATGTGCTTAATTTTGCAAAATGTTTTGCATACCAG
AATGCATTTCTCTTCCAAAAAAGGTACCAGCCTACAAAACCTTGCTGTTACTGTTTTC
AATTAGTTCATGGAATTAAATGTATTAAATGTTTTATGCTCTGGCAGAAATTATGATT
CTCACTTAACTCCATATAAATCTGGATCTGCCTGGGCCTTTATAAGTGACACAATTTC
ATTAACTGAATAAACAAATGATACAAAGAAATTTGGTTTAGCCTTCTAAAATTCCAA
AGGCGTTCAACAAAATATCTCAGAATGGATGTTCCAGGACTTTTATGGCACAGGACA
ACATGTATTGCTTATTTTAAGAAAATAAGCTAAATAGTGAGGGGATTCTTTTAGCAG
ATCCTCAGGATGTGTTAGGTTGAATCATAGGCAAATGATATTTGATCATTGCACCTG
TTAACACATTGAACCTCATCCTAAAATTGTAGAGCTAGAAGAAAGCCTTCTGGCAGT
TTTTAAATAGATTGATTTACTGCAATTTATCCAGAAGCTTCACCGTTGTCACTGGCTA
CATGTGACTTTGGCCTCTGTGGGGCTATATCCTCATTTGTAAAATTGGTGGTGAGGTA
GGTGGACAGTTGACTAAATAATCTCTTAGAATAATTCTAGTATCTGTGGATCTAAAG
CATCCAGGGGTTGAATATGTTTCTTTCTGGCCAAGAAAAGATGCACCTGTCAATAAT
GCCCAAACTCATCTTCTGAGAATCCTCTTTCCCAAGATACCCACTCTCCCTTGGGTTA
TATTATAGTAATGATCAGAAGCCCCTGCCAAGAAGAAACTGTTAACCTGGGAGGTCT
ATATTTTATTTCACAGCCATCTGTTTATACTTTCTCACAAGTTAGTGCACAGTATACC
CATCATTTTCTACCATTTTCCTTAATTTATTAATTTTACTAATTGCATAATTAACAAAA
GTAAGAAGATTTTACCTCCTTATCCCCATCTGGTAGTTTGCAGATACTTGGCCTGATG
ACAACTGACAGTGATGAGATACTCACCAAGTTTACCAGGGCAGGAGGCTTCCTAGA
GAAAAAATGAGAAAATGAAATGGGGAAGGGGAGTGAAGGATTGAGGAGGTGACAA
TCTGGACTCTTGCAACTGCATGGCAAGGTTGGCACACAAGCTGGGTTGCAACGGAGG
GAAGGAGATCCTTATCAGATGTAATCAGAGCTCAGATCGAGGGCTTTGGTGTGTGTA
GAAAGAGGGAGAGACAAAGAACTTAAAACAGAGCTGCCATTTGACCTTGCAATCCC
ATTACTTGGTGTATACCCAAAGGAGAATAAATCATTCTATTAAAAAGACACATGTGC
TTGTATGTTCATGGCAGCACTATTCACAATAGCTAAGACATGGAATCAAACTAGGTG
TCCATCTATGGCAGATTGGATAAAGAAAATGGGGTAAATATAAAGCATGCAATACA
ACATGGCCATAAGAAAAAATGAAATCATGTCCTTTGCTGCAACATGGATGCAGTTGG
GACCCATAATCCTAAGTGAATTAACACAGGAACAGAAAACCAAATACAGCATGTTC
TCACTTATAAGTGGGAGCTAAACACTGAGCACACATGGACATAAATATGAGAACAA
TAAACACTGTGGACTACTAGAGGGGGGAAGGAGAGAGGTTTGTAAAACTACCTATC
AGGTGCTATGCTCAATACCTGGGTGATGGGATTTACACCCCAAACATCAGCATCATT
TAATATTCCCATGTAAAAAGACTGCACATATACCCCTTGTATCTAAAATAAAACTTG
AAATTAAAAAAAAAAGAAAGAAAGAAAGAGGCTGGAAATAGAGGCTCACACCTGT
AATCCCAGCACTTTGGGTGGCCAAGGTGGGTGGATTGCTTGAGCCCGGGAATTCAAG
ACCAGCCTGAGAAACCTGGTGAAACTCTGTCTGTACAAAAAATACAAAAATTATCCA
GGCATGGTGGAGCGCACCTGTAGTCCCAGCTAATGGGGAGGCTGAGGGGGGAACAT
CACTTGAGCCCAGGAGGTGGAGGTTGCAGTGAGCTGGGATCACACCACTGCACTAC
AGCCTGGGTAACAGAGCAACTCTGTCTCAAAGAGAGAGAGGAAAGAAAAAAGAAA
AGATGGACAGATAAGAAAATGCACTTGGAGATTAAGAGAAAGCAGCAACATAGGA
CCCTGGATAATGTGTTTGCTTAATAACTATCCTGATGAGTTATCTGACTATTCCCAAA
TGAGTACGTGGCAATTCAGGCTGAACCATCAGAGTAGCCCTCCGGAATCTTACTTAT
GTACAATAGACCTGCATGCACATTTACTAGAATGAGCCTCTCTCTCTGGTAATCATGT
CTGCTTCCACTAATTCCATCTGTTTCCTCTCTCTCCCTCCTATCCTGCTAGATCTTAAT
TCCTTCGACCTTCCTTTGTTTTTCTAACTCCCTTTCTTTCTCTTGTTATTTAACCTGCTA
TACTATGCAATTGATCTCCTCTGCACTAAGGAACATGCACTTCAGAATTCTGTTGACA
TCTTGCATTCCTTTATATTTAGTGAAAGAATGCAAAGGAGTCTACCTGGCAATATTCA
CTCTGCAGGAGGCAATAATTATTATTCAAATTAAAGGAAGCAGTAAAGAGAAATTC
AGAAAAAATGAAATATACTAATCTTCAGCTTTTCATTTCAGCCTACAAGGAAAAAAT
GAAGGAGCTGTCCATGCTGTCACTGATCTGCTCTTGCTTTTACCCGGAACCTCGCAAC
ATCAACATCTATACTTACGATGGTGAGTAACCTAGGATAGACATACCCCTGCTAGCT
AGATCATTTGGAAAGGTTGACATATATTTGTTTCTTACAGCTCCTGATATAATTACAT
CAATATTTTGTAGCTCTCACTATTGACTTGCCGTGTCTAGCTATTATGTCCAATTGAT
TACCTATTGCTGAAAACAGTTTGAATTTGGTGCTAATAACAACACATCAATGTCTGTT
AAGAAATGTGGATGGATTCTTATTAACAGCCACATCCAGCATATCAACATCCACAAT
ATGTCTAAGGTCTTTCTTTGCAAATAATTTAATAGGCTAAGCCATAATTGGAGTAGA
TCATAATTTGTAAGAAAATGCTTTATACTTAGAAAACTCAAGAGAAAGAATCAACAA
CCATAATTGTTTTTGCTTTATTGTAGTCTTTATAAAGTTTCTATACTTTGTATATACAT
GTCAACCAGCTAATGATAATAATAATTGGCTCAATAAATAAAACTGACTTACGACTG
AGGCCCTAGATAAAGAGGGTCTGAAAAGAAAAGCCTAAAGAATTAGCATGGCAATT
AACATGATTGAGGTGCAACTCTTTAGGTTTGATTTATCCTGATTCATTTTGCTTACTTT
GGCTCTGCCACAATCCACATGATCTTGGTCAAATAGATACTTGGATTCTCTAAGTCTC
ATTTAACTCTAGCATCTTCCTCTTGGAGTTGTTGTGAGGTTTAAACGGTTTAATGTAA
GTCAAATATGCAAAACCAAGCCTAGCTCATTATATCACTCTACAATGATAGCTATCA
TTATCAACATCATCCTTACCTAATTCAGTCAATTTAACTAAAATATTTTATACAGTTC
TATGTATCCTAGATATCCCTAAGGCATATTTTACTAACTCTCAGGCTCACAAATATTT
TTCTTTTCCATATATGTAAAGAAAGACATTAATGACAAAACAAACTGACCTTGTGGC
AGTTAACCCCTTCTGCACCTTTAAAGCCTATTCAAGGACTCAAAGGCATTTACCTTCC
AAAGTTATTCTATCGTAGCACAAAAATCATAAATGCTAATTAACTGTTCCATAAGGA
AATGTCCTCCATGTGAAAGGAATTCTGTCTCCAAACAAAACATTCATTAGAATGCAG
GGCCAATGCCTACTTTGTACAAATTCATTCGGTCAGCAAATAAATTAGACAGACCTT
TATTATTTGCTAGATGTAGCTGTGAAGAAGGATCCAGCTATGTTTCTTATGAGACTA
ATGTCGAACTATGGGTTGTCACTGAGGATCCAGAGTTCCATAGGGCGTAGTCCTCAC
CTTCAAAGAATTCAGGGCTTAGTAGAAGAGTCTTACACAAATGACTAGAATGTAGA
ACACAGAGTGGTTAGGACAAAGGAGCCAGGGATGGTTTTTGCTGGGTTAGGGAATG
AAAAAAGGGGAAGAAAATATGTGAAGTTATGTGTGAGCTGATTCTTGAAATAAGCT
GTTTTTATTTGCCTGCGTTCTCTTATAATCCTTTTCCATAGGCTTCCATAATTTTTATT
GAGCTGTATTTAAAGTTGAATAGATAATTCAACATTTCTCGTAAACTGTGCTTCCTAA
AAGAGTCCGTAGAGAATTTCAAATTTCTGCAGTCTTTAACTTGACCTGGTATTTCTAT
GTTAGATAATAACGTGACTTGTTTATTGCAGGCAAACATTATAACAATAAATTATTA
TTATTGTTTACATTTGTAAGCACTAAGTATATGGCTTGTGCTTTGCATTCAGCATCCT
TTATCATTTAATCTTCACAACCACCTTAGAAGGAAGGTACTCTTTTTATTTCCATCTTT
TAAATGAGGAAATAAAAGCATAAAGAAGTTAATTAACTTACCTAGTGTCACACAGC
TATTAAGAGGGGCTTACTATTTGGATGCAAATATAGGCAGTTCTAATTCCAGAGCCT
CTAATCTAAGGCATTTAAAACCCCATCACCTTATCAAATAAGCTGTTTTTATTTGCCC
GTGTTCTCTTATAATCCTTATCCATAGGTTTCCATAATTTTTATAAAATTGTATTTAAA
ATTTAAGTATAATCTTGGATGCCATCAGGAAAATGAAAAACATTTTTACATTTGTGA
AGGAAAAAGCCCACATCATTTCCAATATAGTTATTGAGTTAGTATTATCTAGACTAT
CTATTAGCAGCTAAGGATCTGAGGTCAAGGCCTGCCAGCCTGGCATTTTACTTGACC
ACAACCTCCATGTGCACTAACCAGGCTGCTAAAAGAACATTAACGGGAACATAACC
TGCTGGCTTGGTTGCCACAATTTTAAAAAGACGTTAATAAATTAGAGAGCACTTAGA
GGTTAGGAAATAATATGGTGGTAAAGATCTAGAAACAGTGTCATTCTGGGGCACTTG
AAGATGTTTAGCCTGGGGGAACAACTTGAAATGGAACATAACTGTTTTCAAATACTT
GAAAAATGGTGGTGCACCACAGAGAATGGCCTAATCATGGGTAGCTTCAGACTTCA
AACAAGGATCAGTGGGCTAAAACCAGAGAGATGGAGTTTGGGACTCAAAGAATGCT
CATCTGAAATTGAGGGCTGACCAGCGAGGTTCTTTTAAAAATCATTGCATTTTACTA
AATTGTGAGTTCTGTAATTATAAATGTCCTAGCAGGTGCTAGCTGTCATCTTTTCTAT
TATAAATTATACTATTTTATGTTATAATTTGTATTATACAGGCTTAAAACATAAGGGT
CTGATAATCTGCTTATCTTTAATACATAAGCCACTGATAGAAAATAAGTGGCTAACC
ATTCTTCAGTTCTTTTTTTAATTGACAAAAATTGTATATGTTTGCGGTGTATGGCATA
TTTTGAAATATGTATACATTAGAGAATGGCTAAGTGAAGCAAATTCACATATGCATT
ACCTCACACACCTGTCATTTATTTGTGATGAGAACAAAAAATCTACTCTTTCAGTGAT
TTTCAAGAATACAGTACATTGTTATTAACAATAGTCAGCATGGTGTACAATAAGTCT
TCTGCGGCCGGGCGTGGTGGCTCACGCCTATAATCCCAGCACTTTGGGAGGCCAAGG
CTGGCAGATCACGAGGTCAGGAGTTCGAGACCAGCCTGACCAACATGCTGAAACCT
TGCCTCTACTAAAAATAGAAAAATTAGCTGAGTGTGGTGGTAAGCGCCTGTAGTCCC
AGCTACTCAGGAGGCTGAGGCAGGAGAATTGCTTGAACCTGGGAGGGGGAGGTTGC
AGTGAGTCGAGATAGTGCCACTGCACTCCAGCCTGGCAAAAGAGGGAAACTCCGTC
TCAATAATAAGTCTCTTGCATTTGTTCTTCCTGTTTAACTGAAATTATGTATTCTTTGA
TCAACATCTCCCCAGTCTCCACCCCTAACCCCTGGTAACCACAATTCTACTCTGCTTC
CGTGAGTTCAACTTTATGAATAGTCCACATGTAAGTGAGATCATGTGGTATTTGTCTT
TCTGTGCCTAGCTTATTTCACTTAGCATAGTGTCCTCCAGGTTCACCCATGTTGTCAA
AAATGACAGGATTTCCCCCAACTTTTTTAAGGCTGAACAGTATTCCATGTGTATGTGT
ATAAATTAGATTAGTAGATGTTGCCACTCCCTCCTCCACCACAGTGGCTCTATCCCTG
GCTCCTGGCTCCAGCCGAGTACACTAGAGGAGGATATTCTAAACAGCAACAACACA
GGAGCAAAGACATTACAATGGGGTGTTGTCTTATTGCCCCCATTAGACTGTAAGCAT
CTTGAAGACAAGGACCCCCATCACAGAGTGATGTTGTCATCCCTGGAGTGGGCACTG
TGCATGATTGATGACTGGAAGCAATGAACATACAGAAGGGCAAAACAGAAATCAGC
AGGATGCTTTGCATTTCAGCATTGACTTTGCCAAATATGCCCAACTGTTCAGGGAGTT
ACATTGGTTCTAACGAAGCTCCTGTGATTCCTAAGCACAGGAATGGTGATAATATAT
ATAATGGTGCATGCATATATACGCATATCTAGATAATGATATCTCATTATATGTGAG
AACTGAAGAACTCCGTTATGTTTCTCGTCTAACCAAAAAGGGCCTACAGCTACGATA
ATTTCCAAACAAATAAATCTGTGCTACTTGATTTTCATGCAAAGCTCATATTTGTTCA
AAAGGAAAATAAAGCTTAATTTAAAATCAATTTAGGCTATTTTTATCTAAGTATGCT
TACCGTTATTCAACTCCCTGCAGATATTGTCAAATTTCTCAATATGGTAAATATTTAT
TCTGTTAAAATATATCCATAGTTACACTAAAGACAGAGAGGTCTTATATGTTCTAAA
CAACATAGAGCAAATGCTCATAAACAGCATTTTATTCCTATCTCCCGGAATAACAAC
GCTACTTCCAATTGCTGGAATCTAAATTATTAAAATAAACCCATGCTGCAAGCTTTGT
ATGCTTAACATTCTCAAATGTTCACTTTTCAGATATGGAAGTGAAGCAAATCAACAA
ACGTGCCTCTGGCCAGGCTTTTGAGCTGATCTTGAAGCCACCATCTCCTATCTCAGAA
GCCCCACGAACTTTAGCTTCTCCAAAGAAGAAAGACCTGTCCCTGGAGGAGATCCAG
AAGAAACTGGAGGCTGCAGAGGAAAGAAGAAAGGTAACTTTTTCCATAGGTTTTCC
TTCTCTCTCTCCCTCCCCTGCTCCTCCCTCTCACACACTCGGGCACACATGCACGCAC
ACACACACACACACACACACACACACACACACACACACACATACAGAGAGCAATGA
CAGCTGAACCTGTGCCATGCCAACATGTATAGGTTTTCAGTAGACACAGAGCCAGGC
TAGTTGGGGTAAAAACTGTAAGATAGATGCTAATTTTAGGCTAGCCAAACCAGAGCT
CTCAGAAATCCAAAGAGCTTCAGTGCTCTAGTGCCCCTTCCCGTATATTGAATCCCCT
TATTATAAAAGCCTCCCTTCCCTAGACCATCAGGCAGAAGCACTGTAGAGAAAACAC
AGCCCTGGCGAACTCCAGTGGTGGGGAGGGGAAGAAGTGCTGCTTCCTCCCTCTCAG
GATCTGTGTCACCCCCTTTGTCAGGCGTGGTTTTCCTTGGAATTACAAATTACCAGAT
CTTCCCTCCAAGATCTTTCCTGCCCAGGGTAAGGGCCAAGAGCTTGCCCCTTTCCTCT
TCAGAGTCCCACTGCCTGCCCTGGAAGTTGGTCCTTCCAAGATCAGGACCTTCTCTG
AGTTCTTTGAATATGTTCTTTATCTTTTTCTAAGACTTGATGGGGATTTTTCTCTTTTT
GCCATTGGTCCCTGCTTATATTAAAGAGCTTTCCTTTTGCCAAATCTTTACTTTTCCAT
AATCACATGGCTAAGAAGAGCCAAGGGTATTATTTGAGAACACTTAGAAATCCTAG
GGACTGTGTACACAAACAGAAGTTGTTTGAATGTGTCTGTTCCAACCATGTGGTTAT
GGTAGTTAATCCCATCAAGGTACTCACGATCATCCAAAAATGGAATTCTTTTATGTA
ATTCATCCCCACATTGTATTTCCCAATATTTTTTATGATATAATTTTAGAATCAGGTA
ATCACTAAGAACATGTTCCCTGCACAGTTTTATGATGTTTTCTCTAAAAAGTCAGCCA
AAACTTTGGACACTTCTATGTTGGATAATTAAAAACAGAATGAAGATAATCCTCCTC
CTAAAGATTGAATTCTCCAAGAGAGAATGCAGGACAAACACAGATGTGCTGTGTAT
AGTATATGTGCATATATACATGCATATATGTACACAAATATGTGTATTATCAAATAA
TGAGGCTCAAACATTAGAAATCCTTAGATTAAATTTTCTAAACAAGAAAACACTAAT
CTTTGTAGTTGAAAAAAAATCCTCCTATGATATGTAATATGCTGATCTCAATTTTCAC
CTAAGAGTGATGTTCTCCAAATGTCCGATGAGCATGTCATATATATATATATGAATTT
TTATATATATAATTACAATGGTAATTGGTATATAGAGATATCTATATTATAGATATAT
ATAGCTATCTCTATATATTACATATACCAATTATAGATATAAATATAACAATGGTAA
CTGGTGTATATGTGATGTGTATATATGTATATGTATACCATAATTATATATTAATATT
GTATATATGCCATAATTATATATTAATATTGGTATATATACACCATGATTATATATTA
ATATTGGTGTGTGTATGTGTGTGTGTATATATATATATATATATAAAATACTAGTTAT
CATTGTTCTAGATTTAAAAAACAGGAACCTGAGCTACTAACTCGACTATATATATAT
ATATATATACAGGAAGTTGCTTTAAAACATTTTTATCAGCTTTTTTATTGTTATTTTTA
GCTTTATTCTCATAGTAAAGCTAAAATAAATTATTCAACATTATCAAAACTTTGCTGC
CAGCAGATGTAAGCAATACCTAAAACAGTGGAGAGCATGTTGCACCCAAAGCAGTT
TAAGCTCTGACCCAAGCACTGGCATCTTATAGGCACTGGGTAGAGATAAGAGTCATA
GGTCGACATATATTGAGATGCTATGACTTGATTAGAATATGGAGTCAGTGACTGAGG
TGAAATTAAAACTCAAACCACAATTCAACATCCTGATTTAGGATGTTGCTGGTGTTT
CTAGGTACTACACTTAATTTGAAAGAAATTATTGAGGATAAAAAAAGAACTGGGAT
CAACAAAATTAACTAGGTGTTCTTATAAGAGTCCCTGAGGTTACTAATTAATGAAAC
TGATAAAGCTCCTGCACCCTGACAGCAAGAAATTATCAATGATTATACATTTAAACA
ATTGAATTGAACTAGAAACTGGCCACATGGTTAAAAGACATTTACAAATGTAATCAT
CCAGTGTTATGATGCCCAGAAAAAAAAAATTCCTTAGAATGCTTTAAAAGCCGTATT
CCATCACCTTTCCAGTTATTTGTTAAACATTTTGTAATGCAAAAATAACCATATAGAT
TATGCCCTAGTGGTCGGGTTTTATTTTTAGTTTTTTATGGTTTTTTTTTGTTAATGGTA
GAGTTTTAATTAAAAGAAAATACAACTAATTAGCAGAAAGTGCCAACTTTAAAAAA
TCACTAATTGATTTTATTCTATTGGGTTATACTGACTTAATTAGCACTAATTTAAAGA
ACTATTAATTATCTTTAAAGAGTCTTTAGCAAGTGCATATATCTCAGTAATTATGTTA
GTAAGGACATGCCTATAACCAAAACCCAACTCAACTAGTTAAAACAAAAAGCAAAT
ATGTGACTAAAAAGTCTAGGAGTGGCTACAGCATCAGGAACAGCTGGATCCAGGGA
TCACAGTATTATCAGAAAACTTTCTTTCAGTGCCTGTCATCTCTTCCTGCATTTAACT
GGTTTCATTATCAAGAAAGTTTAATTTCAATAGTCAGTTCCAAATTATTTTTCTCACA
ACTTAGCAACTCCAGCAGAAACAGAGCTTCTTTTTCCCAATAGTTTAACAAAAGTCC
CGAAATTGAGTCTCAATGGCCTGGCCTGGATCACAGGCCCAACCCAGAACCAATCAT
TATGGCCAAGAGGATGTAGTAGTTTGATATGCTAGCCTGAATCACATGCCCACCACT
GACCTGCAAAGGATTTTAGGTAAGATCCCTGGGGTAAGAATTGTGGAGGGGTAGTTC
CCCAGAAGAAAATCGAGGTGTTCTCACAAGAGGAAGGGGTAATGGATCTTAAATAA
ACAAAACTATAGATGTCCACATTTTCTATCTATAAATGTTTAGTGTTACTATAACAAT
TAGAATAATTATTTAGTTCATACACTATTCAATTTGTATCTCCCTTCTGTTGCCCTGTT
GCCGTTATTTTCTTACAGATAGAATGAAAAATATTAATCTAGGCAGCTCTGTGAAAC
AGTACTGTCCAAGGAATATAACGTGAGCCAGGCCGGGTGTGGTGGCTCATGGCTATA
ATCCCAGCACTTTGGGACGCCGAGGCAGGTGGATCACCTGAGATCAGGAGTTCAAG
ACCAGCCTGGCCAACATGGCAAAACCCCATCTCTACTAAAAATACAAAAATTCGCA
GGGCATAGTGGCGAGTGCCTGTAATCCCAGCTACTGGGGAGGCTGAGGCAGAAGAA
TTGCTTGAACCCAGGAGGTGGAGGTTGCAGTGAACCAAGATGGTACCATTGCACTCC
AGCCTGGATGACAGAGCAAGACTCCATCTCAAAAAAAAAAAAGAAAGAAATGTAAT
GGGAGCCATATGTGTATTTTTAAATGTTCTAGAAGCCACATTTTTTAAAATAAAAGA
AATATGAAATGAATTTTAGTAAAATATTCTTCACCCAATATATTCAAAACATTATTTC
AATATGCATGTAATCAATATAGAAGTATTAATGAGCTGTTTCACATTATTTTATTCAT
ACTAAGTGTTTGAAATCCAGTGTGTATTTTACGTTTACAACTCATTTCAATTCATGTT
AGACATATTCCTAGTGCCTAGTAGCCAAAGGCAGCCAGTAGCACAGATACGGATATT
AAAACAGAAAACACCTAGTGAATAATGGGGAAATTTTAGGCCTAAGTTTTTAAAATC
CATACCAGATAATTATTCAGATTCAAATTTACTTTGTTTTTTCATATATATTCTTTAAA
AATTACATTAATATGGGAACTCAGAAAGTTCAAAAGAAATTTCCATTCTATGGTTTT
AGTCTTTACATTGTCAGAACTAATGCAAGTGTGAAGTTTAGGATGTACTGTAAGTAA
TAGGATCTTCTAAATCTCATGCCTTCTTCAGCTACCTACTCTGTTTCTATTTCAGTTCC
TCACTGTGGGGAGGGGACTTCTCTGAACCTAGGTTTCATCTCTCACTCTCGTTCATGG
TAAACAGGTTTTCCTTTGTGGCACCTAGCACAATTAGTAAGTAATTAGTATTTACTGG
CATATTAGTATATATATGCATATGTATTTATTTAACCCTATGTCTTCTACTAGATTAT
AAACTCCATGAAGATAGAACTTGTCTTTTGTTTAATAGTGCTTGGCAATAGTTATTAC
TGTAAACATTTTTTTTCTTTCTTATTCAACTCCTGTTAGTCATTGCCTGAGTACTACAA
ATGTTTTTAAGTAAATTAATAAATAATAACTTTCAGGGCCAAATGTGAAAGCGGCAA
TATATAGCTTGTTTTGATTTTTTATTCCACCCTCCCATCCTAAAACAATTATAGTCACT
AAGTTTCCAAATGACATCTGAAATTGCACTAAGGAAATCCTAGTCTGGGCAAAATCA
CTCAGTCAACAGATATTTATCAAGCACTTACTATTTGGCAGGCCCTGTTCTAGACAC
AGGGGATACTCATCAAACTTACATTCCAGTGGGGGAGAAAGAGCTAATAAATACAT
ACACAGCATATTAGATGATGCAAAATTAGCAGGACAAAGAGAACTGGGGGTGTGGG
GGTGAAAGAAGCTAATATTATATGTTATTATTACTATATATAATAATATAATTATTGG
ATAGTCAAAAAAAAACCTCTTGAATAAGACATTTGAAAAGAAGCACAAAGGTAGCA
AGGGAGTAGGGCGGGCAGCTCTTCTCTGGGACCTGAACATTCAAAATGATGAGAGC
AGCAGGTGCGGAGGCCCTGAAATAGGAATGTATGAGGTGTGTTTGAGAAATAACAT
GGAGGCCAGCGTGGCTGAAGCTGAGAGCAGGGGGAGAGTGGTAGCAACTGAAGTC
AGAGGTCACAATTAAGGACTTTGACTTCACATGAAATGGGAGATCATGAAGGATAA
TAAAGCCATTTCACTACTTTATGTGAATCACAGCATCTTTTTAAAGAAGTATCCTTTT
TTAAAGGGGGAGATGACTAGAAAAATAAATAGTGTTAGATAAATAGAGAAAACAGG
AAAACATTCTAGACTAAGACAGTGATTCCAGAACTAAGGATCCACAGAGGCGAGAA
TGCAGAAAGTGTAGGTTTCAGAGCAGTGGGTAGACTAAGGGTTTGGACTAGTGGATT
TGGATAGGGAGTTGGAGAGTAGCGAGGTGGGATTAGGGAGGGCTGTGAATGCCAGG
TTAGTGTGCAAACTCCATTATATAAGCAGTAAGGAGTCACTACAGACTTTTCAAAAA
TACATACATGTTCCACCTGGCCCACGGGTTAGCAACATTTTCGTTGCCCTGGACCCAT
TTCCTTCCCAATAAGTTACAGGTTTGTGAAGATTCTACCTAGCAAACATATTACTTTT
AAATAACTATTAATAAATTATCTTACCATGATTATAATCAAAGGAATCTGTAATTGC
TAATTATTTCTGATTATTAAAAGATAAGCAGTATTGCACTAAATTGACATAATTCTAA
CTCAAAGTAAATATACAGATAGACATGGCTATAGATGTGAAATATGATTTCTGTTAG
GGCTTTTTAAATTTAAAAAAACTTACGAGTTCTCCTCCCTCCCCCTACCCTTAATACC
TTGAAGGCCTCTTTGTGGGACTTCAGGGACCCCTTCAGGGAACTATGACCTAGGCTG
TATTTGGGGGGCTTTCTGGGTTTATAGCTGGAAGGCTGCCACAGAGGCATCGCCACT
TGGGCTCAGATTCACTTTGTGTTCAATGTTTTGGCAATGTCCCCACCTCCCCATTCCA
TCTGTTGACACTATTGCAGCACTGACCATCTGGTTACTAGGTTGGAGGATACTCCCTC
GGGCTCCTTTGAACCAGAATTAGTGCTCCAGTGATTAGATAATAGAAGAAGCTTGTC
ATAAAAAGAATAAGCCCTTTCCCTGCTTTTTCTCCATTCTTTGATTATCGCTGGTAGT
CAGTGATGATCATCTCTATGAGTCTATATCAATCTCATCAGGTCAGTTTGAACCTCAT
CTCTTGAAATCAAAGTTTCCATAATGCAACTGACCCACAAGGGTGAAATGACATGAA
TGCTTTAACCATCCATTTATCATTTATTCATTCATTCAACCAACATGTATTTAGCAAG
AGGCAGCAGAGTTAGCATAACTATACATCCCAGTTGGCCCAGGACAACTCCAGCTA
ACTCTCGTTGTTTTGATACCATTATTAATTATTTCTCTTTACTCTCATAAGTGTTCCAC
TTTGGACAATCAATTACATGAGCATCCTTAGCAGGGCACAGTGTTTAAGGGCATCTT
TAAAATATTGTCTTTAAGAACATGTGGTTAAGAGAATGTCTGTGTTCAAATCCTGGTT
CCACCACTTAAAAGCTGTGTGACCTCAAGCAAGTGACTTAATCTCCGTATGTCCTCCT
TTGTCAATCTGTAAAATGAGACTAGTAATAGAACTTATGGAGTTAGTGTGAGAATTG
GAAGGTTACTCTACAATAAAGACATATAACCAGCATGGTAAAAGGGTTAGCAATTA
CTATGTGAAGAAGCATCCAGTTTCTGACCTCACAGAGATTATCTAGCAAACTCATGA
TTTTATAAAGAAAAGAAGTTTCTCATCAACAGAGACTGAAATGCTACCATACAATAT
ACGTTGCTTTTTTTTTTTTTTTTTTTTTGAGACGGAGTCTCGCTCTGCCACTCAGGCTC
AGGCTGGAGTGCAGTGTTGCCACCTTGGCTAATTGCAACCTCCACCTCCCAGGTTCA
AGCAATTCTCCTGCCTCAGTCTCCCAAGTAGCTGGGATTATAGGCACCCACCACCAC
ACCCAGCTAATTTTTATATTTTTAGTAGAGACAAGGTTTTGTCATGTTGGCCAGACTG
GTCTCAAACTCCTGACCTCAGGTGATCCACCCACCTCAGCCTTCCGAAGTGCTGGCA
TTACAGGCATGAGCCACCATGCCCGGCCAATATTTTTAAATATTATAAAATATTCTTT
ATCAAATTGCATAGAAGAAAAGACAGTTTGATAGGTAATAGATATATAAATAGGTC
AGGCCAACTAAAAGTGTCCTGAAAAAATTAATATTGTGAAAACAAAAGGATTTTAA
TGACATTGATAAAATCTCACCCTAAAAGAGATTAAATTAAAAATCACCCTACTTGAA
CCAGTTCAGTGAGATTTCATTAGCATGCTCTCATTACTGGCATAATCAGCTTCAAAGT
CACTAAGCCTCTGAAAGGAAGATGTGTTGCTTATTCTTAATAAAATGGCATAAAAGT
AGATCATTAGTCACCAAACATGATAGACTTACCTTTTCCATTTGTTGGCATCTCACAT
TGTAGATGGCAATTAAAATGGAATCCAGGGAAAGAGGGGGTGGTTTGTATAGCAAT
GGATTATGAAACAAAGTACTGGATTATTCACCGCTTGACATTCAGGAAACATTCTGC
TCCTTACAGAATATGGCACGTGGGCCACAGAATCTTCCGTGTGCTACCTTCTCGGTG
AAGAAGAGCACCCCCAAGTTTCTTTTCCTAGGAGCTAACCACAGTAAACCCATTACA
CACTTTAGCAGAAGGGCTCATTCTAAAGGTCTTAGGATTTTAATCATTTTAAATTTCC
TGTTATGCTTCAGGCTCTTCAACACAAAGTGAATATTGTACTCTTTGGTTTTACATAA
TTATATTCAATTGTCATATTTCAACAGGACATTATTTGTGACTTTAGATGGGTCAATA
ATGATTTTCATTGTCAGCAGTAAAGTCAATAATTACAGACACATCACCTACCCTACTT
GTGTAAAAGCATTTTTTGGTACTAGGAGATTTAGTGTCTGATCAACGGTCCTGGATA
GCAAGTAATATATCCCCCAAATAATGAAAAGTGACAAGAAAATAAATATGTTTACTT
CAGAAATAAATGGAAAATTAGTGCTATCTAAAATGTAGTCTTAAGTCTCATCTGTGT
ACATAAAGTAAAATGAGTTTTATGTACTAGTTACTCAAATTTATCTTCCACTCCATTT
GTATAGTAATTAAACTCTTACACTCAGTAATATACAAATTGGTAATTAACCTCTTTGC
AAAATGTTAAAGTGTTCCTAAATGTACAATAAGTCTCCTTTCCTGTCTCATTGTTTTT
CGCTTCACGTACCTCTCATGTAATTATTTCAATGATTGAGTTCAGTGTGAGGAGGTTT
ATGCCTAGAAAAGGTGCTCACCAATAACGTGCCTCAGTTCCCATAATAGCAAGATCG
AGAAGGTTCTTTAGTCTCCCGGAACGTCACGTTGAACATCTCAGTTCTATATTTTGCC
TTGACATTTGCATTATATCAGCTGATCATTGTCTTGCCCTAATTTTCCCTTTTAATATT
TTAGTGACCTTCTATGTTAGGTACAGGTTATTTAGAAGTGTTCCTCCAAGGCCAGATA
CTTTTTCCTTGAACAATTTATTTTTAACAACTTTTAGCGATTTTCTCACTTCACCACCC
TCCGTTTCATAAGTCCACGCAATCACAATTCCTTTCTGCTAATCTGCACAGTCAAGAT
ATAAAGTAAGAATACCTATTTGAACATGTAGTGAGAACTTTACTTCTCTGCCAAAAA
TGAAGGAAAATGCTGCCACTTTTGTATGTCACATGTTTTTTATTCTACAGCCTCACTC
ACTTCATGTCATGTTTTAGTGCAGTTTTCTGGACTAACTGCTTATTTTCTCATTGATTA
AACTGCCTATTTGCTCATTGGAATTAGAGCCAATTTTTTTCCTTGAGGGTCTGACTAG
AAGATTAAACTATGTTCATGTGAGAATCAATTTCTACCTAAGAAATGAGTTAGAGGA
GTTATGGGCAGCAATATCTATCTGGATGCTACACTGTGAAAAAGGAAGCGAGGTTAT
GCCTTTCTACCCCAATGGGGTAGCAGAGACCTCAGGAACTGAGGTAGATGCCCCCCT
GGTTATTAGCGCCCCTGAATAATTTGTTCAAAAATTGACTGCTGGACAGGTGTCGTG
TTGCACGCCTGTAGTCCCAGCTGTGCAGGAGGCTGAGGCAAGAGGATCTCTTGAGCC
CAGGAATTTGAGGCTATAGTAAACTAAGGTCACACCACTATACTCCAGCCTGAGCAA
CAAAGCAAGACCCTGTCTCTAAATTTAAAAAAAAATATTGAATGCTTATGAATAGAG
ACTAATATAGGAAGTCATAAGTATTTCCTTGGGATAGAATGCTTTCCACCATAATTG
ACTTGACATCCTGTATTTTTGTATGTGTGGACTTAAGTTTTAAATATTTGAAACACAG
ACAATTATTAAGTCCTGCAAATGTGTGAGTTAATAGTGGATATAACATTCCCTTCCA
GGGTGTAAGAAAAGGTACCACAGAAGTGAGCAGCCCTGAAGCACAGCCTGGCCTAG
TTTGGCAGGTCTCTGTGAGTTAGCAGCAGACTCACGTGACCACACTCTGTACTGCCTT
CTGTTTCTGTTTCACCCCATTAATTGTGCTAAAGAAATGCACTTGACACCTATGCTGT
GTAATCTCATTTAGCCCCAATAGCAACAAAAGTACTAACCCCATTAAATTGAGTCAT
TTCAAACTGAGCCAAATGTTGCACTCCAGTAAATGGAGTAGGCATTGGTTATAATGG
GAATTCTCCATTATTCATAATGGAAACCACAGGAGTTTGTTCATGCAGATCAAATGT
GTCCCACCAAGGCAAGAAGTATGGAAAAGTGGTGTTGCTGTATTACCTTGTAATTTC
AAAGCCTTCCCGTCTGAATCTTATTTCCCTGCTGTTTCCTCTTGACTTTGGTTCTTTCA
CAAAGGAAAATTAAGAACACAAATATAAACATTAAGTTAAAACACAACTGAACAAA
GTGCCAAACTTAATTGGAGCATCTGAAAATGAAACATTAGGCAGTTGCAGTGGCCTC
TTGATAATAATTCACAGTAACTCTCTGTAAGCTGATCCTGTCTGAAGAGCAGCAGGC
ACAAGGCCCCTGGCCATGAAGTCCATCTCAAAGGGCCAGGCTCAGCAAAGCAGGAT
GCAAACCCAGGCTTTCCAAATACCAGGTTGGGGCTCATGTCACTGTGCCACAGGAGC
TTCTGTAGAAAGGCTACTTGAAAAAAGTGGCCATTAAAAATCCAGGTGGATCCTATC
TAGGGCAGTGTTGGAAACACTGATCTATGGGAGGAGGAGCAGGAAGGAATTGTTTA
ACCACTGAGCAGAAATGTTACATTGCTACCTGCCTTTAGCAGCTGTGGCTGATGGGT
ACCAGTTGCTAAGAAGAGCATTACCTAACAGTGTATTAAGATAGAAAAATGATTTTA
AAGCACGGCACTTAGAGAATGTTGAAGTTTTACTTTGCTTTATTTTGATTTGTTTGGT
TTGACTTTGTCTCCTGGAGCATCCTCCATGGATTTCTGTTCATTACAAGAGAAACCTA
GGGCTCTAACCCAATTCCTAATTCTTGGACACATTGCACCCTTGTTTTGTGATAATCC
AGCCTTCTTCCTTGAGAAGGTTTGCTGGACTGGAGGTTACATGTATTGAATTTTCTAA
AATGAAGGTGCAAAGCTGTCTCCTCTTATTTCTTTGTGGTGCTCACTTCACTGTGAGA
TTTCCTATCAATACAGCCCAAGTCAGTGGGCATGCATGAGGTGGAGATGAGGGAGTT
AGGAAGGACTTGGACTCTCATCAACCATCAGGATCCCTGAATCCACTAACTGTTCAT
AATCAAAGAAGTTTGAACAAATACTTCACACACATGAAATTGCCAAAATTTTGCATT
TGAGTTGTTATACCAGTAAGTCCAGTTGCCATCATCTCCTTGTCACAAGTGTCTTAAA
TTTTGCTTTTGATAATAATGATTACCACTCATTCAGTACTAACTTACTTGATATTAGA
CACTGCATTAAATACCTTGCAAACATTATTTTGTTTGATCCTGACAACCATATGAGAT
AGGTACTATTCTTATCCATTACCAAAAAAATTAATTTCATGAAGACTTTTCCCAGAG
AGAGAAACTTTAAATATTTACACACACACCTCTCTCCCTGTAACAATTCCGTAGTCCT
GATAACAGCAAATAAGCAAAGTCTGTGTAGGATGCTTTACCAACAGTCCCACCTAGA
GGCAGGAGAGTGAACCAGCTAGAAAATATTTTATTCATATTTCTTCCAGAAAGGCTC
CATTGGAGTTTGAACTCAATTTATGTTATAATTTTCTTATTATTTTTGTATTGGTTTTC
CTGAAACCAATACAAAGTAAGAAAGCATTGGTTCCACTAAAAATGTCCTAAAACCA
GCCAAGCACAGTGGCTCACACCTATAATCCCAGTACTTTGGGAGGCCGAGGCGGGT
GGATCACTTAAGCCAGGAGTTCAAGACTAGCCTGGCCAACATGACGAAACCCCATCT
CTACTAAAAATACAAAAATTAGCAGGGTGTGGTAGCACACACCTGTAATCTCAGCTA
CTCAGGAAGCTGAGACATGAGAATCGCTTGAACCTCAGAGGCAGAGATTACAGTGA
GCAGAGATCACGCCACTGTACTTCTGCCTGGGTGACAGAGCGAGACTCTATCTAAAA
AAAAATAAACACATAAATAGTAAAATGTCCTGAAACCATTATGGGGTTAAAGCAAG
AGGCAGGGCTGGTTCCCAGGATTTTCTGTCTAATCTCCAGTGAGCCACAGACCTATT
CCTGATCAACTTGAGAATAAACACATCAGTAAAGATGTGTAAGGCTGTCTGACTTTC
CCATTTCTGTAGAATTTTATTTGAAGAGAAGTTTCTCCTTTCTCCAGGCCCCATATTG
TTTATACAAAAAGACCTTTCCAGTAAATGTCCACAACCACTACCATCAACTAAAATG
TTTTCCCACTAATGCTTTCAATGGTAATCAGTATTTAACAGGGCACTTAGGATTATTT
TTTGATCAACCATTGTTTAGATATTCCCACTTATAATTACTCCTGTGAAGGATTGCCT
CGGGGCATCAGCTGATCCTGAGAAATTATCCAGAAGCCATGAGTGTGTAATAATTTA
GTCTTAAACCTAAATAGGTCAGTATTGGGTGGGACTTTTCTCAGCTGCATAATGGGG
AGAATAAAAAGAATATGGAAAGAAGTTACGTAACACATCCTGGGTCACAAACAGAG
GTAAGACTTGAACACAGGCCTGACATCAAAGCCCATGCCAGTATGACTTACAAAAG
GTAGACTGGACTACCTGCATTTGAGTCACTAGTGATGCTTATCACTGGGCCTCACCA
AAGAACCTTGGAATCAGAATCTTTGGAGGTAGATGCCAGGCACCTGCATTGTTATCA
AGTGCTCCAGTGATTACCATTCACTGTACAGAGCCAAACAGACTCCTGATGCTGGAA
GAAAATTACAGTGCTCAAAGTGCAGGGCAGGGTGTACATCTGGATCTAAATCACTG
AGCAACCACAGGGTTTCAAGAGAGGGTCAAAACAAGGACTTTCTGCTCTCTGTGGCC
AAGGGGACACTAAGTTTGCACTGTTCTCAGATCTCCAAAGAGACTTTGGTGTATGGG
GGATAGGGAGGGGGGAAGGGGGTGTGAAATAAAAGGAGAAAGTGAATTTGATTATT
TGATTGATGAAAATTGAAAAGCTTATTGTAGGGCCTAGCCTACAGTTGATGAAAAAA
CAATGGATCAGGAAGAAGATCAGAACTTGTCTCAGTCCTCAACTGTTTTCCTCAGGC
TTTGGTTGAATATTGCCATCCTGTAATTCATTATAGCATTTTCTGTTGCATAAACGCT
TAGCAACAAAGCCTTTTTTTAAAAAAATTTGTAACTCCTCAATGAGGATTAAATGCT
TCTTCTTCTAAGACAGTCCGAAATATACTCACAGCTGAAAATTCAGCTAACCGCATT
TCCCAACTAGCCACATTCTATAGAAAACTCTAAGCCATGCAGATGAGTACAGACTTG
ACAATAGTGCTCAAGGCTGGGAGTACTATTCATCTGAAAAGAATGCTCCCTCCAATT
GGTGGGCCGTTATTCTGCTAGGTTTGTGTTTGGATAATTATAAGATGGCTATGTTTTT
CTTCCCCAGTCTCAGGAGGCCCAGGTGCTGAAACAATTGGCAGAGAAGAGGGAACA
CGAGCGAGAAGTCCTTCAGAAGGCTTTGGAGGAGAACAACAACTTCAGCAAGATGG
CGGAGGAAAAGCTGATCCTGAAAATGGAACAAATTAAGGAAAACCGTGAGGCTAAT
CTAGCTGCTATTATTGAACGTCTGCAGGAAAAGGTAATCTCAGCAGAGTCCTGAGCA
GATGGATATATTCATATGCAGCACAGCTGGGTGAACTTCCATATGCCTGAGCACAGA
GACGAAGTCAAAATTTGCTGCAGGTGTGAGGACAACTAACTCCCATGGGCAGGGTC
TCACAGTGTAGCATTGAGTTAGCAGGAGGTGCAACATGGTAGAGAAATGGGAATCC
ATCATGAAAGCTGGAATTTTGTCAAATTTTCCCATGGTGAGTGGATTCAGGGAGGCT
GATTCATGCTTTTGAAATGTGTAAGACTTCTATACAAGCCTCACGAGGCAATCTGTA
GGAAAAATGTTACACTGGAAATATTAATGTCTATATATTATATTGATATAAGTATAA
ATAACATTTGATTTAATATTTGTTTAATATATGACATTAAATATATATTTAATTAAAA
TATTAAATTAGAAAAATATATTTGCCAGAAAAGGCCAGGGTATTTATGAACACTGGT
AAGCCCATTCTAGGGTATAATAGCATCACATGGGACCATAGCAAAGATTAGCTCATA
GGGGATGTTTCATCCAGTTCTGGTATCCTGGTGCCCTTCTCTTCAACAACCTAAACAT
ATATTCATTCCCATGAGTCAGGAGGAGCTGTGCTGGAGTTCTTCTGAAAAATGCTGT
CTTTCACTTTTGTACTCTCTATGCTGTCTCCCACCTATCCCCTCAAAAAACCTTTCCTT
TGAAAATATACAGTATAGCTGTGAGTAGTTTAGCTGTGTCCGTTTCCAGAAATTGGA
ATAAGCATTGAGAAATGGGATGTTTGAGAAAGACGCCTCAATCCTTTTCTGAGCAGT
CAGTCACCCTTCCCGCCAGTAGCAAGTGCCTTTGTGTGATAGGCATTGGAGATGCAG
AGCAAAACAGGAGTGTGCCTGTCATCAGAGCCCTGAGAGTTTAATTAGATGAGCCTC
CTGTTTTCTATTTCTCAGAGTTTCATGTCTTCTGTTAGAGATGGCCCTTCTCATCTAAG
GTTCAAAAAACCTTATCCTGAAGTTCTGATGATTCTGTTTTCATTCTCAGTCTCTGAC
TGCAAATATCCAACTAGAAACAAAGGAAATCAGGCATGAAAACTTTTAAAGATATA
ATTGCATGGAGATCTTCATTTGTGCTCGTGAGGAATTTTTGAAAGCATTGCTGGGGA
AGGGTGTGTGGGCTCTGATGCAGCAGTAAGACACTGAGGCTCTCAGAGGTCCGTGG
ACGAGTACTGCTGACTTGGGCAAGAACCGGAATAGTTACCTGATGCCTTATCCGAAA
CATGAAAGTTCGGATTAAATTTGTATTTATAAGCTAGTGTTTTTATACTCTCAGAACA
ATGTCATTGCGTTTCACCCAAGTGAGTCAAGTCACGATTTGGAAGAGGCAACAGAAT
TTGGCTCTCTCCAGGTGATTTATGGCGGTATAGGAACACATGTTTTACTCAGATACA
GGGGAGCAAAGTTCCATTTGCTAAAGTTTACTCCCCTGACCTTCAACCAGTCAGTCTT
CCTCCATCTGCCACCACTTTGCACTTCTCCAGAGAACTAAGGATGTTCCCGCTTGACC
AGTGCTCATAACATGGACAGCAGAGGGCCACTGTGTGATCTCTTTGAGATCACTGTG
ACTCAACCTTCTTCTCACATCCTAGGCCCTAAAACAATTAAGTGAAGTTGCTAGGAA
CGGTACCTGCTGATCTTATTGCAGCATTCTCAATTAGGCCTCAATGCAAGATTTATAT
CACTGGCAGTCCTGGAGCATTTTTGTTTTTCAAATTACACATACCCAAACACACGGC
ATAGCCTCCTTTTTTGTTTGTTTGTTTTTTTGAGATAGAGTCTCGCTGTGTCGCCCAGG
CTGGAGTGCAGTGGCACGATCTCAGCTCACTGCAACCTCTGCCTCCTGGGTTCAAGT
GATTCTCATGTCTCAGCCTCCCAAGTAGCTGAGATTACAGGCGTATACCACCACGCC
CAGCTAATTTTTGTATTTTTAGTAGAGACAGGGTTTTGCCGTGTTGGCCAAGCTGGTC
TCAAACTCCTGACCTCAAGTGATCCACCCACCTCGGCCTCCCGAAGTGCTGGGATTA
CAGGTGTGAGCCACCGTGCCCAGCCAGGGCATATCCTTCTTGATTTCAATTGTAAAA
TAGTTCAAAAATTTTCCATATTTTATCTAATATTTCCAGAAGTGCTAGCTTTTAACGG
ACCATTTTTTTCCTCTGTGTGTTTTTTTCTCTTCACCTAGCCCAGCCATGCTCAGCTCA
TTTTTGTACTCTTTCCACTCCCAACCAAATTTAGTGCCCTCCCCCATACATGCATACA
TGTACATCTGCACACCACTTTTCCTGCAAATAATCAACCCAAAGAGTGCTTAAAATT
CCTGACATCAACCCACAGAATCTCCAAGGATGGGACCCAGCATCCATACATTTTAAA
AACTCTCCATATAGTTCCAATATGCAGCCAGATTTGAGAACTAGTGGTTCGTAGCCT
GTTCTGATTTAAATCTCAGCTCTCAGCAGTCTATCCCACGTCACATAATGCAGCCCAG
AGAAATTCTAGGACCACATTTTTTTCTGGTATTTCATAGCTAATGAGGTGCTTTTCAA
ATCTAATAGGATCTTTGGCCAGTGTCAGTCAAGATCTTTTATCTCCTCAATAAAAAG
GAAATACCATATTTACTTTGATTTGATGTATATCACATAGGTGGATTTAATACAAAAT
TGTGGTTTACATATTGTGAATGTGTATACTAAAACTACTTTGCTTTTTCCTAAAATAA
GACAAAGTTTTATATTGGAAGTAATATTTAGCATTTTGTTTGAATGAAGTTACTCCTA
TTAAATTAGAAATTTAAAAGAGGGTCAGTAATAACAGTAAAGCCAAAAGGCATGAC
ACTGCCAACGTAACATAAGCTGCTCTGAAATCTACCATATCAAAAGATAATTATGCT
GGGCATGGTGGCTCACACCTGTAATCCCAGCACTTTGGGAGGCCAAGGCAAGAGAA
TTGCTTGAAGCCAGGAGTTCGAGACCAGCCTGGGAAATATAATGATACCTTGCCTCA
AACAAAAATTCAAAAATTAGCCAGCAGTGGTGGCACACTTGTAAAAATGCCTGTAG
TCATAGCTACTTCAGAGGCTGAGATGAAAGGATTGCTTGGGCCAAGGAGTTCGAGA
CTGCACTCCAACCTGGGAAATATTGTGCCACTGCACTCCAACCTGGGAAACAGAACA
AGACCCTGTCTCTAAAATAAAAAGAAAAAAAAAGATGACCACTTCTGAAATGACAC
CTATCAATGAGTTAATCATTCAATGAATATGTATTGAGTCCCTACTATATGCTTAGGA
ACCTTTGTAATATCATTACCAACCATGTCTTTCCCAATACAGACAATACAAAATTCA
GCAATAAATAATATAGCACCAACAATTAGAGAATAAGACAACATGTAGTATGGTCC
AATATAGACAGTAAATACAAAGACACTGAATAATATCAGTAAAAGTAAATTCACAT
CAAGGTCACTACACCATGCGCCCACCCTTATGATAGCCCTCACTGGCCCTATCAATT
AAGCAAGAGACATGATACAACTCTGTGCAAGCTTTTCCACAATCTGCCTACCATTCA
GCACTCAGTCGCTCTTCCCTTCAATTAAGAGAATTGAGCATTCAAGCATATTTTCACC
ATGATGCCCATAATGGTATCTTCAATGTCACTGACTGATAAATTCCCAGAAACCCCT
CAGAGCCCCAGCCATGTTAGCTCAAAGCCTTTAGCTAAAACTGAAAGCCTAAAGCA
AAAGCAGCCCTGGCTGCACTTCGGAATCTACTGGACAGCTCTTTAAGGGATTCTGAT
TTAATGTCTGGAATAGGGCCAAGAACCTTGTATTATTTTAAAGGCTCACTAGTAGGC
TCTAATATTTAGCCGTGGTTGAGAACCACTGTGCTAAATGTTTCTTAAATATGCTTTG
TGATGTCATCATAAATTATATTTTAGTATTTTTTGTCTTTGTTGCATAAGTGTTCTTTC
TTCCTCCAAAGAAGAATGTTACACTCATTTCTTATTTCAGTTTCCTGTTTTCATAGCA
CCTCATCTTAACACTCCAGGCTATTATATAGAAAAGAATCAAATGTGGAGAAGGCTG
TGGGAGAAGGGATGCCTGTGCCACAAAGGCCTGCATTAGGCTGACCTATTGATGTCA
TATCCAGGACTCAAAAGACTAGTCTGTGGATTATGACTGGTGAAGTTCAAAATGTTC
TTATTCTTAGAGTGGTATGAGAAGTAGAAAGAGAGAGAAACAGAGAAGGGGAGGA
GAGGGGAAGAGAGGAAGATGAGAGAAAGGAAAGAGAGGGGGAAACACCTGTTCTT
GACATACAGGAATGATTCAAGACATTTTCTTCCTCCCCTGATGTGTCCCTTTCTCCCC
TAACGCACTATGCAGCATCCTGCAGAAAATTCACCACCTGACCCTTTTAGAAACCCT
GAGTAGTAGGAGCGCCAAATGACCCAATCAAGAATTGCAGTGAGACAGTTAGTTTT
GAAAAATCAGTTAAAGCATGTATAATCATTTTAACAACAATACATCTATTCACTAAA
CATATAATTTTAATGTCAAATATTTACGTGTAAACATATTGACCAATCTTTCGATGTA
GTTGGGCCCAATACCTTTTCCAAAAATTGATCAGTTAATGGGGGTTCTATGGGGGTT
TCTTTTCTTGCCATTATTCACACTTATGTCACATTAGCTATGATTTGCAGTTTTAATTT
CTTTAAAATTGAGTAGGGACTAAAGACATCTCCAAAAAGCCTGGATATAGACTTTTT
ACAACTTTTCCATAGCTTTTATAGTTGACTCACCCAGTATCTACTAAATACTTCACTT
TCTCACGTATTTCCAAAGGTTTCTCTCCACCCTCACAATTTTCCATTAATGTAGTACTT
AATTAAATTAGATAGTTAAATTTTCAAATGTGAATTGCTAAACAGGTGTGGAAATAC
CATTGGCTATAATCAAGCATATAACACAACCATTTGAGAAGGAAAGTATGTGGCAAT
ATTAGGGAAGAGCCCTTTCCTCTCAAGCAATTCAGCATTTAGGAACCATCAGACAGC
AGGACGATGGAGGGAACAGAGAGGGTTAACATGGCAAGTTACTGAAGAGGACTTCT
ACTGAATCTTGTTGAATTCCCCACTTAATCCAGATTGTATCATATCTTCTTTCTTTTGT
AATTCTACCATATCATCTTAGTCAATGCCAAGACTTCTGAGCTCATAACATGGTAAC
AAATACCAAAGGAGCTTTCAGTATCGTTTAGAAAGGAGAGAAGCAAGTAACCCAGA
CAAACTTGACAACTGCTTTCCCCTATCCAACCATGAAGTACAGTACTTAGGAAATAA
AAGAAATTGCTTCACTATAATTCATCATTTCACTTCTAATATCTAGAAAATGTCAAAT
GAAAATATTATAGCCATATTTTAGTGGCAATAGTAGCACATAATATGATGCAACTTA
AAATGATAAAAATATTTTCAGGGAATAAGATTCTGTGATTCTTTCCCTAAGAGGTAA
TTTTGATAATATGTACCTGTTTTGTAAATGTCAATAGTCTTGGGGATACAGGTGGTGT
TTGGTTACATGGAAAAGTTCCTTAGTGGTGATTTCTGAGATTTTAGTGCACCCAATAC
CCAAGCAGTGTACACTGTACCCAATATGTAGTCTTTCATCCCTCGCCCCCACTCCCAA
CCTTCCCCCACAAGTCTCTAAAGTCCATTATATCACTCTTATATCTTTGCATACTCAT
AGCTTAGCTCCCACTTATGAGAACATATGATAGTTAGTGCTCAATTCCTGAGTTACTT
CACTTAGAATAATGGCCTCCAGCTCCACCCAAGTTGCTGCAAAAGACACTATTTAGT
TCCTTTTTATGGCTGAGTAGTATTACATGGTGTATATATACCACATTTTATTTATCCA
CTTGTTGGTCAATGGACACTTAACATTAGTTCCATATCTTTGTAATTTCAAGTTGTGC
TGCTATAAGCATGCATGAGCCTGTGTCTTTTTCATATAATTACTTCTTTTCCTTTGGGT
AGATACCCAGCAGTGGGATTGCTGGATCAAATGATAGTTCTACTTTCAGTTCTTTATG
TTTTCCACAGTGGTCATACTAATTTACATTCCCATCAACAGTGTAAAGTGTTCCCTTT
TCATCACACCCATGCCAACACCTATTGTTTTTTGACTTTTTAATTACGGCCATTCTTGC
AGGAGTAAGGTGGTATTTCATTGTGGTTTTAATTTGCATTTCCCTGATGTTGACAATA
TTTAACTCTTTAGTTATAGATTCCAGCTATTATCAATTTACACCTATTGCATTCTTCTC
ATCTTTTGTTTTCTTGTGATTCTGATGCACAAATATCATTTGTGCAACCACTTACTGTT
GAACATGTCTGATGAACACTTACTATTGAACATGTCTGATGAATGAATAATGAAATA
GGAAAAGGGATTAAAACTAGCCTTTATTAATTGTTTGCTATAGGCCAGACATTTTTG
GATGTACTATCACATTTCATCCAAACAACAACCTAAAAGAAAATACTGTGATTATCC
CCATTTCACATCTAAGGAACCTGGTCTTTAGGAAGATTAAGTCATTTGGCCAAGATC
ACAAGTAGACCACAGAGACTAGATTTGAATGCAAGTCTGTTTGACTCCAAACCTTTT
TACTATCTGCCCATGACCCCTGATCACCAACATCTCAATGTATGAACATGTGCTTTCT
TAGCTCACACAACTCACTCCTGACCCCTTTTTTATATTGCAAGTGCATAGTCATTAGT
AAAAAGAAGGATTTTTGATGATACTGACCTCATCTTGAATTTAATTAGGCTCATATG
ACAGAATTCCATAGATGGAATTGACATCCTAGGTCATATAGTCCAAGTCCTTGTTTA
TATTTGATACCTAGTGAGATTAAAGGGACATTAAAAAGTAAAGAAAGGAAAGACCT
CATATTTCTTACCTTCCAGTAGAGAAATCTTTCTATGAAATCAGAGGAAAGAATTAG
AGGACCAGAATTTTTCCTAAAATCAACTTTCATACATCTTTTTTCATATAAAAGGCAT
AGCTGCATACAATGCTAAAATATTGTATTACATTTCCTTTATATTGATGGGAGGAAG
GGGGTAAATTGCAGAAAACATTGTAAATTTAGATATGCTTGGGCCTCTGACAGTGCC
TAGCAAATATCAGGAGATCAATAATGAAATAAATATTATCAAAGAGTAGTCTTCTTG
ATGAACCTTCTCTGAGTATCACAACTGCTTTAGGAACCTCTAGATTCAAGGTCTAGT
AATTGCAAACAGTGAGCTGATAAGAAAAACAGACTGTATGGGAAATTACATGCTTC
CTGCATGACTGCCTTTTGTTCTCCCACATTTTGATATAAAGTCACATTAACAGTTCAT
GAGTAAATATTCGATAATGTGAACGTAAAGTGTTCAAATAATAGAGTGACTAAAAT
GCCTGAAAACAAATAATTTTTAATTAGAAACTCATAATCATTTATTTTCTCTTTTTCC
ACATTATCTCAAGCTCACAAATTATATTTATTCTTTCCTATGGCAAAATCCATTTTGT
TAACACTAATTTTGAGTTTAACAAGAAGTGTACTCCAAAGTAGCCTAATAATACTAA
TTATAATGTTTCCTGCTATGTTATCAGTTTGAATTTATATGAATCTTTAGACTTGAGG
CTTCTTTTTCCTAGCATAGTGATGGTCTGGGCTTTTTCTCAATTTTTGCCAGAGCTCAG
CTCTCACTAATTAGTTTCTTTCTGCATGAGAAAAAGATTTTGCTTCATCTTTTTCCTTA
TAATAGCAGAACAAAAAGAAGAATCAGCTGCATCCATGCTAATTTCCCCTGTGACAT
TTCCAAACAGGATTTGATTTCTCTATGCATGCCTCTTTCCTTCTCTTCATGGTTTTTGA
ACATATACAAAAGCTCATTTAAACCAATTAAATAAAATTGTTTTTAATCTCTTTCTCT
AGAGTCAACTTCCTGCTTACTCCAACTCTGTATCTTTGAAGGAAGTATAGGGTGGTCT
ATGCCTTTTTTCTCCCAGAATCTACACTTGAAAAGACACATTTTTCCATGCAACTATA
AAATGTTCTCCTCACTCAACATTGAAATTGTATAGCAGTGATTAAGAGAGTGAGCTG
TAGAGCCAGGTTCCCTGGGTTTAAATCCCACTTGTTAGTATCATGAAGATGGGCAAG
TTACTTACCCTTCCTGTGTTTCAGTTTCTTCATCTGCAAAATGGGGACAATAATAGAA
TGTCCACTATAAGATTATTGTGAGGATTAAGGGAATTAATACAGGTAAAACGTGTAC
TGATGCAGGTCTGGTACACATTAAGTGCCTAATAAATATTCAGTATTATGATATAAA
GAACCCTATAAGTGTAGACTCCTTGAGATTAATAGAGTTTAACGATAAGTTTTACTTT
ATAGCTGGTCAAGTTTATTTCTTCTGAACTAAAAGAATCTATAGAGTCTCAATTTCTG
GAGCTTCAGAGGGAAGGAGAGAAGCAATGTAAGCAACATTCTACAGAAATATAAAT
AATACTACTAATAATTAGCATCTTAAAATTTCAATTCAATGAACATTTATTTAGCGCC
TATGATATATGCAAGACAGTTTGATTTTAGTCATCTGATGTATAGCCACATACTAAA
AAATACTGATTTTAGTCATCTGATGTATAGCCACATACTAAAAAATACTTCCTCCATC
AGTTCCCTCCTCAGGAAGTTCAGTTCCCAATCCCAGGCTAGTACCTTGGTTCCTTATG
TAAATAAACATCCACCAATTACATGCTATCTGCAAAGCACTCTGCTAGGCCCTGCAA
ATGGAAAAAAAAATGATAAAACATAGTCCAGGCCCTCAATGAGCTTACAGTCAAAT
ATAATAGAGGAGACAAGAACAGAGAGGCTCATAATACAACTAGAATAAAATGACTG
CCGAATAAAAGGAAAGATTTATGCAGGTGTTCAAATGGAAAGTGAGATAAGTTTGC
AGGTTAGTCTTTGCAGTCTCATAAAAATCTTTATGGAGAAAAGGACAATGGTCATAG
GGCTTAAAGAGTAAGTTTATAATCCTGACCAGTGGAGATGAAAGACTAGCATTGAA
AATTGCATGACAAGACAATTCCATTAAACTGAAACATCAAGTGTGTGTAGGAAAAG
ATGGGGGTTATGACTGGAAACGTCACTTGGACTGCAATTATGAAGGGCCTTGACAAA
CAGGTCAAGAGTTTAAGAAGCAGTATAGAAAGTCTTCGTCCTGGATCTAGCCCTCCC
AGAGTGTCCATCAGGATTATAAAGTCCTTAAAATATTAGTCAAAAGGAACGACATCA
TTAGAAATGATAGAGAAACAATAATGTGATGTTTTATTACCTTTCTCTGGATTTATAC
TCTGATCCTAATATTCAAAACTATCTTAATAACATGAACTTTTGGTCATAGTTTTAAA
CAAAAACAGTGTTAAATATATTTTTTAAAACACAGTAAGTCTTGTAAGATCTTTTCTA
ACATGACATTTTGCAGGGCCCATATTTTCCTTCTGAAATGGGAAAAATTCATAAAAG
TAGACACCAAACTGGGTTACTTCTAGTCAAGCGCATGGTACGCAAAGGACCAGACA
AAAAGGGCCTGTGACATTTCTTCTTCCTTTTGTGTTTTTTAGGAGAGGCATGCTGCGG
AGGTGCGCAGGAACAAGGAACTCCAGGTTGAACTGTCTGGCTGAAGCAAGGGAGGG
TCTGGCACGCCCCACCAATAGTAAATCCCCCTGCCTATATTATAATGGATCATGCGA
TATCAGGATGGGGAATGTATGACATGGTTTAAAAAGAACTCATTATAAAAAAAAAA
AAACAAAAAAAATCAAAAATTAAAAAAAATCAATGCGGTCTCTTTGCAGAATGTTTT
GCTTGATGTTTAAAAAATACCTTGGATCTTATTTTGTAAATACTTACATTTTTGTTAA
AAAATACAAGTATTGCATTATGCAAGTTATTTCATAATCTTACATGTCCTGTAACAG
GCTTTTGATGTTGTGTCTTTCCACTCAAATGAATTTGCTAGGTCTGTTCTTTTTGAAGC
TCCCCATGTCTAACTCCATTCCAAAAGAAAAATGAGGTCAGTAGACAGTCTATGGTG
CTAGAAACCCACCATTGCCTAATGACCTAGAAGGCTTTGTTGTCTCTGAGCTTGACT
AAGACCATACCTAGATCACAGGTATTATGACTCCACATGAACCTTCACATTTGTTCG
CTCATAATCTACTTACTGCCTAAAAACTACAAAACCAGGCTAAGAAATACCACCAGT
CATAGCATTTACTTCTGCTTCTCCTGGATTATGTGCTACAAATGTGCTTTGGCTTTAG
AAAGGGATGGATGAGAAGACAGACCTGAGACCAATCTGGGTAGAAGCAAAAAGTT
GAACCTTTTAAAGTGCTGAACACAAATCCAAATTCGAATGGTTCAAGCAGCCGTGAA
ATCGCTCTTCATAAAGTGGGCTTAATTCTCTAGTTTAAGTTCTTTTGATGGAATGAAT
TAATTAATGTGTCAGGTGGCTTATTTGTGGATGCCATGATTGATGATGTTCATTTTAA
GCTCTTACCTATAGTACAAGTACATGATGCTACTGAATATTTTTCCACTTGGAAACTG
TGAGCTGGTTGTTGCATTAAAACACACATACAAACAAAATCAAAAACACTGCGGAC
TTTCACTCAAGCTGGTCTTTCTTCCCCAGTGTAAGGCAATCCTGCCTACTAACAACAC
CAACAACAAAACACTCCATCTGTGAAGCTGACGCAGTTAAGGGGGCTAGGCAGGGC
ATTTGTGCCAACTAAGAATCACCAGATACCCACCATAAGTACCTATCGCAGTTTTGA
AGTCGTTTCTCCCCAACTCCCAACTCCTGAAGGTTGCTGCCTGCATATTTACTCTTCA
TTAGTGCTATTTTCCTGTATGTCATTGTGAGCAAGCTGTGATTAATAAAGAATTGGAG
TTCTGTGAACTAATAAAGGTTTGGTCTGTT

### STMN2 Oligonucleotides Targeting Regions of the STMN2 Transcript

In various embodiments, STMN2 AON disclosed herein are complementary to specific regions of STMN2 transcripts (for example, a STMN2 pre-mRNA comprising a cryptic exon) comprising a sequence that shares at least 90% (e.g., 90%, 91%, 92%, 93%, 94%, 95%, 96%, 97%, 98%, 99%, or 100%) identity to SEQ ID NO: 1341. In some embodiments, a STMN2 AON comprises a sequence that is complementary to a specific region of the STMN2 transcript (for example, a STMN2 pre-mRNA comprising a cryptic exon) comprising a sequence that shares at least 90% (e.g., 90%, 91%, 92%, 93%, 94%, 95%, 96%, 97%, 98%, 99%, or 100%) identity to SEQ ID NO: 1339 or SEQ ID NO: 1341. In some embodiments, a STMN2 AON comprises a sequence that is at least 85% complementary to a specific region of the STMN2 transcript. In some embodiments, a STMN2 AON comprises a sequence that is 90 to 95% complementary to a specific region of the STMN2 transcript.

In some embodiments, the STMN2 AON (e.g., STMN2 AON) has a segment that has, at most, 7 linked nucleosides. In some embodiments, the STMN2 AON has a segment that has, at most, 6, 5, 4, 3, or 2 linked nucleosides. The segments of the STMN2 AON may be separated from other segments of the STMN2 AON through a spacer. The segment of the STMN2 AON is complementary to a specific region of the STMN2 transcript (for example, a STMN2 transcript comprising a cryptic exon) comprising a sequence that shares at least 90% (e.g., 90%, 91%, 92%, 93%, 94%, 95%, 96%, 97%, 98%, 99%, or 100%) identity to SEQ ID NO: 1339 or SEQ ID NO: 1341.

In some embodiments, a STMN2 AON targets a specific portion of the STMN2 transcript, the specific portion of the STMN2 transcript comprising any one of positions 144-168, 173-197, 185-209, or 237-261 of SEQ ID NO: 1339. In some embodiments, a STMN2 AON targets a specific portion of the STMN2 transcript, the specific portion of the STMN2 transcript comprising any one of positions 121-144, 146-170, 150-170, 150-172, 150-170, 150-172, 150-174, 169-193, 170-194, 171-195, 172-196, 197-221, 249-273, 252-276, or 276-300. In some embodiments, a STMN2 AON targets a specific portion of the STMN2 transcript, the specific portion of the STMN2 transcript comprising any one of positions 144-164, 144-166, 145-167, 146-166, 146-168, 147-165, or 148-168 of SEQ ID NO: 1339. In some embodiments, a STMN2 AON targets a specific portion of the STMN2 transcript, the specific portion of the STMN2 transcript comprising any one of positions 173-191, 173-193, 173-195, 173-197, 175-195, 175-197, 177-197, or 179-197 of SEQ ID NO: 1339. In some embodiments, a STMN2 AON targets a specific portion of the STMN2 transcript, the specific portion of the STMN2 transcript comprising any one of positions 185-205, 187-209, 189-209, or 191-209 of SEQ ID NO: 1339. In some embodiments, a STMN2 AON targets a specific portion of the STMN2 transcript, the specific portion of the STMN2 transcript comprising any one of positions 237-255, 237-259, 239-259, 239-261, 241-261, or 243-261 of SEQ ID NO: 1339.

In some embodiments, a STMN2 AON targets a specific portion of the STMN2 transcript, the specific portion of the STMN2 transcript consisting of any one of positions 144-168, 173-197, 185-209, or 237-261 of SEQ ID NO: 1339. In some embodiments, a STMN2 AON targets a specific portion of the STMN2 transcript, the specific portion of the STMN2 transcript consisting of any one of positions 144-164, 144-166, 145-167, 146-166, 146-168, 147-165, or 148-168 of SEQ ID NO: 1339. In some embodiments, a STMN2 AON targets a specific portion of the STMN2 transcript, the specific portion of the STMN2 transcript consisting of any one of positions 173-191, 173-193, 173-195, 173-197, 175-195, 175-197, 177-197, or 179-197 of SEQ ID NO: 1339. In some embodiments, a STMN2 AON targets a specific portion of the STMN2 transcript, the specific portion of the STMN2 transcript consisting of any one of positions 185-205, 187-209, 189-209, or 191-209 of SEQ ID NO: 1339. In some embodiments, a STMN2 AON targets a specific portion of the STMN2 transcript, the specific portion of the STMN2 transcript consisting of any one of positions 237-255, 237-259, 239-259, 239-261, 241-261, or 243-261 of SEQ ID NO: 1339.

### STMN2 Oligonucleotide Variants

In various embodiments, STMN2 AONs include different variants, hereafter referred to as STMN2 AON variants. A STMN2 AON variant may be an oligonucleotide sequence of 5 to 100 nucleobases in length, for example, 10 to 40 nucleobases in length, for example, 14 to 40 nucleobases in length, 10 to 30 nucleobases in length, for example, 14 to 30 nucleobases in length, for example, 16 to 28 nucleobases in length, for example, 19 to 23 nucleobases in length, for example, 21 to 23 nucleobases in length, for example, or 18, 19, 20, 21, 22, 23, 24, 25, 26, 27, 28, 29, or 30 nucleobases in length. A STMN2 AON variant may be an oligonucleotide sequence complementary to a portion of a STMN2 pre-mRNA sequence or a STMN2 gene sequence.

In various embodiments, a STMN2 AON variant represents a modified version of a corresponding STMN2 parent oligonucleotide that includes a nucleobase sequence selected from any one of SEQ ID NOs: 1-446 or SEQ ID NOs: 893-1338. In some embodiments, a STMN2 AON variant includes a nucleobase sequence that represents a shortened version of a nucleobase sequence of a STMN2 AON selected from any one of SEQ ID NOs: 1-446 or SEQ ID NOs: 893-1338. As one example, if a STMN2 parent oligonucleotide includes a 25mer (e.g., 25 nucleotide bases in length) a variant (e.g., a STMN2 variant) may include a shorter version (e.g., 15mer, 17mer, 19mer, 21mer, or 23mer) of the 25mer STMN2 parent oligonucleotide. In one embodiment, a nucleobase sequence of a STMN2 AON variant differs from a corresponding nucleobase sequence of a STMN2 parent oligonucleotide in that 1, 2, 3, 4, 5, or 6 nucleotide bases are removed from one or both of the 3′ and 5′ ends of the nucleobase sequence of the STMN2 parent oligonucleotide. In one embodiment, the corresponding STMN2 AON variant may include a 23mer where two nucleotide bases were removed from one of the 3′ or 5′ end of a 25mer included in the STMN2 parent oligonucleotide. In one embodiment, the corresponding STMN2 AON variant may include a 23mer where one nucleotide base is removed from each of the 3′ and 5′ ends of the 25mer included in the STMN2 parent oligonucleotide. In one embodiment, the corresponding STMN2 AON variant may include a 21mer where two nucleotide bases are removed from each of the 3′ and 5′ ends of the 25mer included in the STMN2 parent oligonucleotide. In one embodiment, the corresponding STMN2 AON variant may include a 21mer where four nucleotide bases are removed from either the 3′ or 5′ end of the 25mer included in the STMN2 parent oligonucleotide. In one embodiment, the corresponding STMN2 AON variant may include a 19mer where three nucleotide bases are removed from each of the 3′ and 5′ ends of the 25mer included in the STMN2 parent oligonucleotide. In one embodiment, the corresponding STMN2 AON variant may include a 19mer where six nucleotide bases are removed from either the 3′ or 5′ end of the 25mer included in the STMN2 parent oligonucleotide.

Example sequences of STMN2 AON variants are shown below in Tables 5A and 5B.

TABLE 5A
STMN2 Oligonucleotide Variant Sequences

| SEQ ID | AON Sequence* | SEQ ID | Target Sequence |
|---|---|---|---|
| NO: | (5′→3′) | NO: | (5′→3′) |
| 1342 | ATCCAATTAAGAGAGAGTGATGG | 1367 | CCATCACTCTCTCTTAATTGGAT |
| 1343 | AATCCAATTAAGAGAGAGTGATG | 1368 | CATCACTCTCTCTTAATTGGATT |
| 1344 | TCCAATTAAGAGAGAGTGATGGG | 1369 | CCCATCACTCTCTCTTAATTGGA |
| 1345 | GAGTCCTGCAATATGAATATAAT | 1370 | ATTATATTCATATTGCAGGACTC |
| 1346 | GTCCTGCAATATGAATATAATTT | 1371 | AAATTATATTCATATTGCAGGAC |
| 1347 | GTCTTCTGCCGAGTCCTGCAATA | 1372 | TATTGCAGGACTCGGCAGAAGAC |
| 1348 | GCACACATGCTCACACAGAGAGC | 1373 | GCTCTCTGTGTGAGCATGTGTGC |
| 1349 | ACACATGCTCACACAGAGAGCCA | 1374 | TGGCTCTCTGTGTGAGCATGTGT |
| 1350 | TCCAATTAAGAGAGAGTGATG | 1375 | CATCACTCTCTCTTAATTGGA |
| 1351 | AATCCAATTAAGAGAGAGTGA | 1376 | TCACTCTCTCTTAATTGGATT |
| 1352 | CAATTAAGAGAGAGTGATGGG | 1377 | CCCATCACTCTCTCTTAATTG |
| 1353 | GTCCTGCAATATGAATATAAT | 1378 | ATTATATTCATATTGCAGGAC |
| 1354 | GAGTCCTGCAATATGAATATA | 1379 | TATATTCATATTGCAGGACTC |
| 1355 | CCTGCAATATGAATATAATTT | 1380 | AAATTATATTCATATTGCAGG |
| 1356 | AGGTCTTCTGCCGAGTCCTGC | 1381 | GCAGGACTCGGCAGAAGACCT |
| 1357 | CTTCTGCCGAGTCCTGCAATA | 1382 | TATTGCAGGACTCGGCAGAAG |
| 1358 | ACACATGCTCACACAGAGAGC | 1383 | GCTCTCTGTGTGAGCATGTGT |
| 1359 | GCACACATGCTCACACAGAGA | 1384 | TCTCTGTGTGAGCATGTGTGC |
| 1360 | ACATGCTCACACAGAGAGCCA | 1385 | TGGCTCTCTGTGTGAGCATGT |
| 1361 | CCAATTAAGAGAGAGTGAT | 1386 | ATCACTCTCTCTTAATTGG |
| 1362 | GAGTCCTGCAATATGAATA | 1387 | TATTCATATTGCAGGACTC |
| 1363 | TGCAATATGAATATAATTT | 1388 | AAATTATATTCATATTGCA |
| 1364 | TCTGCCGAGTCCTGCAATA | 1389 | TATTGCAGGACTCGGCAGA |
| 1365 | GCACACATGCTCACACAGA | 1390 | TCTGTGTGAGCATGTGTGC |
| 1366 | ATGCTCACACAGAGAGCCA | 1391 | TGGCTCTCTGTGTGAGCAT |

*At least one nucleoside linkage of the nucleobase sequence is selected from a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

TABLE 5B
Additional STMN2 Oligonucleotide Variant
Sequences

| SEQ ID NO: | AON Sequence* (5′→3′) |
|---|---|
| 1421 | CCTGCAATATGAATATAATTTTA |
| 1422 | TGCAATATGAATATAATTTTAAA |
| 1423 | CTGCAATATGAATATAATTTTAA |
| 1424 | TGCAATATGAATATAATTTTA |
| 1425 | TCCTGCAATATGAATATAATTTT |
| 1426 | CTGCAATATGAATATAATTTT |
| 1427 | AGTCCTGCAATATGAATATAATT |
| 1428 | TCCTGCAATATGAATATAATT |
| 1429 | TTTCTCTCGAAGGTCTTCTGCCG |
| 1430 | CCTTTCTCTCGAAGGTCTTCTGC |
| 1431 | CTTTCTCTCGAAGGTCTTCTGCC |
| 1432 | CTCTCGCACACACGCACACATGC |
| 1433 | CTCTCTCGCACACACGCACACAT |
| 1434 | TCTCTCGCACACACGCACACATG |
| 1435 | CTCTCGCACACACGCACACAT |

*At least one nucleoside linkage of the nucleobase sequence is selected from a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

Table 6 below identities additional variants of STMN2 AON sequences:

TABLE 6
Additional STMN2 Oligonucleotide Variant
Sequences

| SEQ | AON Sequence* |
|---|---|
| ID NO: | (5′→3′) |
| 1392 | AUCCAAUUAAGAGAGAGUGAUGG |
| 1393 | AAUCCAAUUAAGAGAGAGUGAUG |
| 1394 | UCCAAUUAAGAGAGAGUGAUGGG |
| 1395 | GAGUCCUGCAAUAUGAAUAUAAU |
| 1396 | GUCCUGCAAUAUGAAUAUAAUUU |
| 1397 | GUCUUCUGCCGAGUCCUGCAAUA |
| 1398 | GCACACAUGCUCACACAGAGAGC |
| 1399 | ACACAUGCUCACACAGAGAGCCA |
| 1400 | UCCAAUUAAGAGAGAGUGAUG |
| 1401 | AAUCCAAUUAAGAGAGAGUGA |
| 1402 | CAAUUAAGAGAGAGUGAUGGG |
| 1403 | GUCCUGCAAUAUGAAUAUAAU |
| 1404 | GAGUCCUGCAAUAUGAAUAUA |
| 1405 | CCUGCAAUAUGAAUAUAAUUU |
| 1406 | AGGUCUUCUGCCGAGUCCUGC |
| 1407 | CUUCUGCCGAGUCCUGCAAUA |
| 1408 | ACACAUGCUCACACAGAGAGC |
| 1409 | GCACACAUGCUCACACAGAGA |
| 1410 | ACAUGCUCACACAGAGAGCCA |
| 1411 | CCAAUUAAGAGAGAGUGAU |
| 1412 | GAGUCCUGCAAUAUGAAUA |
| 1413 | UGCAAUAUGAAUAUAAUUU |
| 1414 | UCUGCCGAGUCCUGCAAUA |
| 1415 | GCACACAUGCUCACACAGA |
| 1416 | AUGCUCACACAGAGAGCCA |
| 1436 | CCUGCAAUAUGAAUAUAAUUUUA |
| 1437 | UGCAAUAUGAAUAUAAUUUUAAA |
| 1438 | CUGCAAUAUGAAUAUAAUUUUAA |
| 1439 | UGCAAUAUGAAUAUAAUUUUA |
| 1440 | UCCUGCAAUAUGAAUAUAAUUUU |
| 1441 | CUGCAAUAUGAAUAUAAUUUU |
| 1442 | AGUCCUGCAAUAUGAAUAUAAUU |
| 1443 | UCCUGCAAUAUGAAUAUAAUU |
| 1444 | UUUCUCUCGAAGGUCUUCUGCCG |
| 1445 | CCUUUCUCUCGAAGGUCUUCUGC |
| 1446 | CUUUCUCUCGAAGGUCUUCUGCC |
| 1447 | CUCUCGCACACACGCACACAUGC |
| 1448 | CUCUCUCGCACACACGCACACAU |
| 1449 | UCUCUCGCACACACGCACACAUG |
| 1450 | CUCUCGCACACACGCACACAU |

*At least one nucleoside linkage of the nucleobase sequence is selected from a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

### KCNQ2 Oligonucleotides Complementary to KCNQ2 Transcript

In some embodiments, a KCNQ2 AON includes a sequence that is at least 85% complementary to a sequence that shares at least 90% (e.g., 90%, 91%, 92%, 93%, 94%, 95%, 96%, 97%, 98%, 99%, or 100%) identity to a region of a KCNQ2 transcript (e.g., any one of SEQ ID NOs: 3032-3045). In some embodiments, a KCNQ2 AON includes a sequence that is between 90-95% complementary to a sequence that shares at least 90% (e.g., 90%, 91%, 92%, 93%, 94%, 95%, 96%, 97%, 98%, 99%, or 100%) identity to a region of a KCNQ2 transcript. (e.g., any one of SEQ ID NOs: 3032-3043). In particular embodiments, a KCNQ2 AON includes a sequence that is between 85% and 90% complementary to a sequence that shares at least 90% (e.g., 90%, 91%, 92%, 93%, 94%, 95%, 96%, 97%, 98%, 99%, or 100%) identity to a region of a KCNQ2 transcript (e.g., any one of SEQ ID NOs: 3032-3043). In particular embodiments, a KCNQ2 AON includes a sequence that is between 84% to 88% complementary to a sequence that shares at least 90% (e.g., 90%, 91%, 92%, 93%, 94%, 95%, 96%, 97%, 98%, 99%, or 100%) identity to a region of a KCNQ2 transcript (e.g., any one of SEQ ID NOs: 3032-3043). In particular embodiments, a KCNQ2 AON includes a sequence that is between 89% to 92% complementary to a sequence that shares at least 90% (e.g., 90%, 91%, 92%, 93%, 94%, 95%, 96%, 97%, 98%, 99%, or 100%) identity to a region of a KCNQ2 transcript (e.g., any one of SEQ ID NOs: 3032-3043). In particular embodiments, a KCNQ2 AON includes a sequence that is between 94% to 96% complementary to a sequence that shares at least 90% (e.g., 90%, 91%, 92%, 93%, 94%, 95%, 96%, 97%, 98%, 99%, or 100%) identity to a region of a KCNQ2 transcript e.g., SEQ ID NOs: 3032-3043).

In various embodiments, a KCNQ2 AON comprises a sequence that shares at least 85% identity with an equal length portion of any one of SEQ ID NOs: 1676-1851, SEQ ID NOs: 2028-2529, SEQ ID NOs: 3046-3221, SEQ ID NO: 3398-3899, and SEQ ID NOs: 4402-4530. In various embodiments, a KCNQ2 AON comprises a sequence that shares at least 90% identity with an equal length portion of any one of SEQ ID NOs: 1676-1851, SEQ ID NOs: 2028-2529, SEQ ID NOs: 3046-3221, SEQ ID NO: 3398-3899, and SEQ ID NOs: 4402-4530.

In some embodiments, the KCNQ2 AON comprises a spacer and has a segment having at most 7 linked nucleosides. In some embodiments, the KCNQ2 AON comprises a spacer and has a segment having at most 6, 5, 4, 3, or 2 linked nucleosides.

KCNQ2 AON binding specificity can be assessed via measurement of parameters such as dissociation constant, melting temperature or other criteria such as changes in protein or RNA expression levels or other assays that measure KCNQ2 activity or expression.

In some embodiments, a KCNQ2 AON can include a non-duplexed oligonucleotide. In some embodiments, a KCNQ2 AON can include a duplex of two oligonucleotides where the first oligonucleotide includes a nucleobase sequence that is completely or almost completely complementary to a KCNQ2 pre-mRNA sequence and the second oligonucleotide includes a nucleobase sequence that is complementary to the nucleobase sequence of the first oligonucleotide.

In some embodiments, a KCNQ2 AON can target KCNQ2 mRNAs of one or more isoforms. In some embodiments, the KCNQ2 AON includes a nucleobase sequence that is complementary to a nucleobase sequence of a KCNQ2 gene or a KCNQ2 miRNA.

KCNQ2 AONs described herein include antisense oligonucleotides comprising the oligonucleotide sequences listed in Table 7A and 7B below:

TABLE 7A
Example KCNQ2 AON Sequences complementary to a sequence in the Intron 4
region of a KCNQ2 transcript.

| SEQ ID | AON Sequence* | SEQ ID | Target Sequence |
|---|---|---|---|
| NO: | (5′→3′) | NO: | (5′→3′) |
| 1676 | CGCCAGCCTTGGGGCCGTGACTCAC | 1852 | GTGAGTCACGGCCCCAAGGCTGGCG |
| 1677 | CCGCCAGCCTTGGGGCCGTGACTCA | 1853 | TGAGTCACGGCCCCAAGGCTGGCGG |
| 1678 | ACCGCCAGCCTTGGGGCCGTGACTC | 1854 | GAGTCACGGCCCCAAGGCTGGCGGT |
| 1679 | CACCGCCAGCCTTGGGGCCGTGACT | 1855 | AGTCACGGCCCCAAGGCTGGCGGTG |
| 1680 | CCACCGCCAGCCTTGGGGCCGTGAC | 1856 | GTCACGGCCCCAAGGCTGGCGGTGG |
| 1681 | CCCACCGCCAGCCTTGGGGCCGTGA | 1857 | TCACGGCCCCAAGGCTGGCGGTGGG |
| 1682 | GCCCACCGCCAGCCTTGGGGCCGTG | 1858 | CACGGCCCCAAGGCTGGCGGTGGGC |
| 1683 | CGCCCACCGCCAGCCTTGGGGCCGT | 1859 | ACGGCCCCAAGGCTGGCGGTGGGCG |
| 1684 | GCGCCCACCGCCAGCCTTGGGGCCG | 1860 | CGGCCCCAAGGCTGGCGGTGGGCGC |
| 1685 | GGCGCCCACCGCCAGCCTTGGGGCC | 1861 | GGCCCCAAGGCTGGCGGTGGGCGCC |
| 1686 | GGGCGCCCACCGCCAGCCTTGGGGC | 1862 | GCCCCAAGGCTGGCGGTGGGCGCCC |
| 1687 | GGGGCGCCCACCGCCAGCCTTGGGG | 1863 | CCCCAAGGCTGGCGGTGGGCGCCCC |
| 1688 | GGGGGCGCCCACCGCCAGCCTTGGG | 1864 | CCCAAGGCTGGCGGTGGGCGCCCCC |
| 1689 | TGGGGGCGCCCACCGCCAGCCTTGG | 1865 | CCAAGGCTGGCGGTGGGCGCCCCCA |
| 1690 | CTGGGGGCGCCCACCGCCAGCCTTG | 1866 | CAAGGCTGGCGGTGGGCGCCCCCAG |
| 1691 | GCTGGGGGCGCCCACCGCCAGCCTT | 1867 | AAGGCTGGCGGTGGGCGCCCCCAGC |
| 1692 | GGCTGGGGGCGCCCACCGCCAGCCT | 1868 | AGGCTGGCGGTGGGCGCCCCCAGCC |
| 1693 | TGGCTGGGGGCGCCCACCGCCAGCC | 1869 | GGCTGGCGGTGGGCGCCCCCAGCCA |
| 1694 | CTGGCTGGGGGCGCCCACCGCCAGC | 1870 | GCTGGCGGTGGGCGCCCCCAGCCAG |
| 1695 | GCTGGCTGGGGGCGCCCACCGCCAG | 1871 | CTGGCGGTGGGCGCCCCCAGCCAGC |
| 1696 | CGCTGGCTGGGGGCGCCCACCGCCA | 1872 | TGGCGGTGGGCGCCCCCAGCCAGCG |
| 1697 | TCGCTGGCTGGGGGCGCCCACCGCC | 1873 | GGCGGTGGGCGCCCCCAGCCAGCGA |
| 1698 | CTCGCTGGCTGGGGGCGCCCACCGC | 1874 | GCGGTGGGCGCCCCCAGCCAGCGAG |
| 1699 | TCTCGCTGGCTGGGGGCGCCCACCG | 1875 | CGGTGGGCGCCCCCAGCCAGCGAGA |
| 1700 | CTCTCGCTGGCTGGGGGCGCCCACC | 1876 | GGTGGGCGCCCCCAGCCAGCGAGAG |
| 1701 | ACTCTCGCTGGCTGGGGGCGCCCAC | 1877 | GTGGGCGCCCCCAGCCAGCGAGAGT |
| 1702 | GACTCTCGCTGGCTGGGGGCGCCCA | 1878 | TGGGCGCCCCCAGCCAGCGAGAGTC |
| 1703 | GGACTCTCGCTGGCTGGGGGCGCCC | 1879 | GGGCGCCCCCAGCCAGCGAGAGTCC |
| 1704 | AGGACTCTCGCTGGCTGGGGGCGCC | 1880 | GGCGCCCCCAGCCAGCGAGAGTCCT |
| 1705 | CAGGACTCTCGCTGGCTGGGGGCGC | 1881 | GCGCCCCCAGCCAGCGAGAGTCCTG |
| 1706 | CCAGGACTCTCGCTGGCTGGGGGCG | 1882 | CGCCCCCAGCCAGCGAGAGTCCTGG |
| 1707 | GCCAGGACTCTCGCTGGCTGGGGGC | 1883 | GCCCCCAGCCAGCGAGAGTCCTGGC |
| 1708 | GGCCAGGACTCTCGCTGGCTGGGGG | 1884 | CCCCCAGCCAGCGAGAGTCCTGGCC |
| 1709 | GGGCCAGGACTCTCGCTGGCTGGGG | 1885 | CCCCAGCCAGCGAGAGTCCTGGCCC |
| 1710 | TGGGCCAGGACTCTCGCTGGCTGGG | 1886 | CCCAGCCAGCGAGAGTCCTGGCCCA |
| 1711 | CTGGGCCAGGACTCTCGCTGGCTGG | 1887 | CCAGCCAGCGAGAGTCCTGGCCCAG |
| 1712 | TCTGGGCCAGGACTCTCGCTGGCTG | 1888 | CAGCCAGCGAGAGTCCTGGCCCAGA |
| 1713 | GTCTGGGCCAGGACTCTCGCTGGCT | 1889 | AGCCAGCGAGAGTCCTGGCCCAGAC |
| 1714 | GGTCTGGGCCAGGACTCTCGCTGGC | 1890 | GCCAGCGAGAGTCCTGGCCCAGACC |
| 1715 | CGGTCTGGGCCAGGACTCTCGCTGG | 1891 | CCAGCGAGAGTCCTGGCCCAGACCG |
| 1716 | CCGGTCTGGGCCAGGACTCTCGCTG | 1892 | CAGCGAGAGTCCTGGCCCAGACCGG |
| 1717 | CCCGGTCTGGGCCAGGACTCTCGCT | 1893 | AGCGAGAGTCCTGGCCCAGACCGGG |
| 1718 | GCCCGGTCTGGGCCAGGACTCTCGC | 1894 | GCGAGAGTCCTGGCCCAGACCGGGC |
| 1719 | GGCCCGGTCTGGGCCAGGACTCTCG | 1895 | CGAGAGTCCTGGCCCAGACCGGGCC |
| 1720 | GGGCCCGGTCTGGGCCAGGACTCTC | 1896 | GAGAGTCCTGGCCCAGACCGGGCCC |
| 1721 | GGGGCCCGGTCTGGGCCAGGACTCT | 1897 | AGAGTCCTGGCCCAGACCGGGCCCC |
| 1722 | TGGGGCCCGGTCTGGGCCAGGACTC | 1898 | GAGTCCTGGCCCAGACCGGGCCCCA |
| 1723 | GTGGGGCCCGGTCTGGGCCAGGACT | 1899 | AGTCCTGGCCCAGACCGGGCCCCAC |
| 1724 | GGTGGGGCCCGGTCTGGGCCAGGAC | 1900 | GTCCTGGCCCAGACCGGGCCCCACC |
| 1725 | GGGTGGGGCCCGGTCTGGGCCAGGA | 1901 | TCCTGGCCCAGACCGGGCCCCACCC |
| 1726 | GGGGTGGGGCCCGGTCTGGGCCAGG | 1902 | CCTGGCCCAGACCGGGCCCCACCCC |
| 1727 | AGGGGTGGGGCCCGGTCTGGGCCAG | 1903 | CTGGCCCAGACCGGGCCCCACCCCT |
| 1728 | CAGGGGTGGGGCCCGGTCTGGGCCA | 1904 | TGGCCCAGACCGGGCCCCACCCCTG |
| 1729 | GCAGGGGTGGGGCCCGGTCTGGGCC | 1905 | GGCCCAGACCGGGCCCCACCCCTGC |
| 1730 | GGCAGGGGTGGGGCCCGGTCTGGGC | 1906 | GCCCAGACCGGGCCCCACCCCTGCC |
| 1731 | AGGCAGGGGTGGGGCCCGGTCTGGG | 1907 | CCCAGACCGGGCCCCACCCCTGCCT |
| 1732 | CAGGCAGGGGTGGGGCCCGGTCTGG | 1908 | CCAGACCGGGCCCCACCCCTGCCTG |
| 1733 | CCAGGCAGGGGTGGGGCCCGGTCTG | 1909 | CAGACCGGGCCCCACCCCTGCCTGG |
| 1734 | CCCAGGCAGGGGTGGGGCCCGGTCT | 1910 | AGACCGGGCCCCACCCCTGCCTGGG |
| 1735 | CCCCAGGCAGGGGTGGGGCCCGGTC | 1911 | GACCGGGCCCCACCCCTGCCTGGGG |
| 1736 | ACCCCAGGCAGGGGTGGGGCCCGGT | 1912 | ACCGGGCCCCACCCCTGCCTGGGGT |
| 1737 | AACCCCAGGCAGGGGTGGGGCCCGG | 1913 | CCGGGCCCCACCCCTGCCTGGGGTT |
| 1738 | AAACCCCAGGCAGGGGTGGGGCCCG | 1914 | CGGGCCCCACCCCTGCCTGGGGTTT |
| 1739 | CAAACCCCAGGCAGGGGTGGGGCCC | 1915 | GGGCCCCACCCCTGCCTGGGGTTTG |
| 1740 | GCAAACCCCAGGCAGGGGTGGGGCC | 1916 | GGCCCCACCCCTGCCTGGGGTTTGC |
| 1741 | AGCAAACCCCAGGCAGGGGTGGGGC | 1917 | GCCCCACCCCTGCCTGGGGTTTGCT |
| 1742 | AAGCAAACCCCAGGCAGGGGTGGGG | 1918 | CCCCACCCCTGCCTGGGGTTTGCTT |
| 1743 | GAAGCAAACCCCAGGCAGGGGTGGG | 1919 | CCCACCCCTGCCTGGGGTTTGCTTC |
| 1744 | TGAAGCAAACCCCAGGCAGGGGTGG | 1920 | CCACCCCTGCCTGGGGTTTGCTTCA |
| 1745 | TTGAAGCAAACCCCAGGCAGGGGTG | 1921 | CACCCCTGCCTGGGGTTTGCTTCAA |
| 1746 | CTTGAAGCAAACCCCAGGCAGGGGT | 1922 | ACCCCTGCCTGGGGTTTGCTTCAAG |
| 1747 | TCTTGAAGCAAACCCCAGGCAGGGG | 1923 | CCCCTGCCTGGGGTTTGCTTCAAGA |
| 1748 | CTCTTGAAGCAAACCCCAGGCAGGG | 1924 | CCCTGCCTGGGGTTTGCTTCAAGAG |
| 1749 | GCTCTTGAAGCAAACCCCAGGCAGG | 1925 | CCTGCCTGGGGTTTGCTTCAAGAGC |
| 1750 | GGCTCTTGAAGCAAACCCCAGGCAG | 1926 | CTGCCTGGGGTTTGCTTCAAGAGCC |
| 1751 | GGGCTCTTGAAGCAAACCCCAGGCA | 1927 | TGCCTGGGGTTTGCTTCAAGAGCCC |
| 1752 | AGGGCTCTTGAAGCAAACCCCAGGC | 1928 | GCCTGGGGTTTGCTTCAAGAGCCCT |
| 1753 | CAGGGCTCTTGAAGCAAACCCCAGG | 1929 | CCTGGGGTTTGCTTCAAGAGCCCTG |
| 1754 | CCAGGGCTCTTGAAGCAAACCCCAG | 1930 | CTGGGGTTTGCTTCAAGAGCCCTGG |
| 1755 | CCCAGGGCTCTTGAAGCAAACCCCA | 1931 | TGGGGTTTGCTTCAAGAGCCCTGGG |
| 1756 | ACCCAGGGCTCTTGAAGCAAACCCC | 1932 | GGGGTTTGCTTCAAGAGCCCTGGGT |
| 1757 | CACCCAGGGCTCTTGAAGCAAACCC | 1933 | GGGTTTGCTTCAAGAGCCCTGGGTG |
| 1758 | CCACCCAGGGCTCTTGAAGCAAACC | 1934 | GGTTTGCTTCAAGAGCCCTGGGTGG |
| 1759 | TCCACCCAGGGCTCTTGAAGCAAAC | 1935 | GTTTGCTTCAAGAGCCCTGGGTGGA |
| 1760 | CTCCACCCAGGGCTCTTGAAGCAAA | 1936 | TTTGCTTCAAGAGCCCTGGGTGGAG |
| 1761 | CCTCCACCCAGGGCTCTTGAAGCAA | 1937 | TTGCTTCAAGAGCCCTGGGTGGAGG |
| 1762 | CCCTCCACCCAGGGCTCTTGAAGCA | 1938 | TGCTTCAAGAGCCCTGGGTGGAGGG |
| 1763 | TCCCTCCACCCAGGGCTCTTGAAGC | 1939 | GCTTCAAGAGCCCTGGGTGGAGGGA |
| 1764 | ATCCCTCCACCCAGGGCTCTTGAAG | 1940 | CTTCAAGAGCCCTGGGTGGAGGGAT |
| 1765 | CATCCCTCCACCCAGGGCTCTTGAA | 1941 | TTCAAGAGCCCTGGGTGGAGGGATG |
| 1766 | CCATCCCTCCACCCAGGGCTCTTGA | 1942 | TCAAGAGCCCTGGGTGGAGGGATGG |
| 1767 | TCCATCCCTCCACCCAGGGCTCTTG | 1943 | CAAGAGCCCTGGGTGGAGGGATGGA |
| 1768 | CTCCATCCCTCCACCCAGGGCTCTT | 1944 | AAGAGCCCTGGGTGGAGGGATGGAG |
| 1769 | ACTCCATCCCTCCACCCAGGGCTCT | 1945 | AGAGCCCTGGGTGGAGGGATGGAGT |
| 1770 | GACTCCATCCCTCCACCCAGGGCTC | 1946 | GAGCCCTGGGTGGAGGGATGGAGTC |
| 1771 | TGACTCCATCCCTCCACCCAGGGCT | 1947 | AGCCCTGGGTGGAGGGATGGAGTCA |
| 1772 | CTGACTCCATCCCTCCACCCAGGGC | 1948 | GCCCTGGGTGGAGGGATGGAGTCAG |
| 1773 | ACTGACTCCATCCCTCCACCCAGGG | 1949 | CCCTGGGTGGAGGGATGGAGTCAGT |
| 1774 | CACTGACTCCATCCCTCCACCCAGG | 1950 | CCTGGGTGGAGGGATGGAGTCAGTG |
| 1775 | CCACTGACTCCATCCCTCCACCCAG | 1951 | CTGGGTGGAGGGATGGAGTCAGTGG |
| 1776 | ACCACTGACTCCATCCCTCCACCCA | 1952 | TGGGTGGAGGGATGGAGTCAGTGGT |
| 1777 | CACCACTGACTCCATCCCTCCACCC | 1953 | GGGTGGAGGGATGGAGTCAGTGGTG |
| 1778 | CCACCACTGACTCCATCCCTCCACC | 1954 | GGTGGAGGGATGGAGTCAGTGGTGG |
| 1779 | GCCACCACTGACTCCATCCCTCCAC | 1955 | GTGGAGGGATGGAGTCAGTGGTGGC |
| 1780 | AGCCACCACTGACTCCATCCCTCCA | 1956 | TGGAGGGATGGAGTCAGTGGTGGCT |
| 1781 | GAGCCACCACTGACTCCATCCCTCC | 1957 | GGAGGGATGGAGTCAGTGGTGGCTC |
| 1782 | AGAGCCACCACTGACTCCATCCCTC | 1958 | GAGGGATGGAGTCAGTGGTGGCTCT |
| 1783 | CAGAGCCACCACTGACTCCATCCCT | 1959 | AGGGATGGAGTCAGTGGTGGCTCTG |
| 1784 | CCAGAGCCACCACTGACTCCATCCC | 1960 | GGGATGGAGTCAGTGGTGGCTCTGG |
| 1785 | GCCAGAGCCACCACTGACTCCATCC | 1961 | GGATGGAGTCAGTGGTGGCTCTGGC |
| 1786 | AGCCAGAGCCACCACTGACTCCATC | 1962 | GATGGAGTCAGTGGTGGCTCTGGCT |
| 1787 | CAGCCAGAGCCACCACTGACTCCAT | 1963 | ATGGAGTCAGTGGTGGCTCTGGCTG |
| 1788 | CCAGCCAGAGCCACCACTGACTCCA | 1964 | TGGAGTCAGTGGTGGCTCTGGCTGG |
| 1789 | TCCAGCCAGAGCCACCACTGACTCC | 1965 | GGAGTCAGTGGTGGCTCTGGCTGGA |
| 1790 | CTCCAGCCAGAGCCACCACTGACTC | 1966 | GAGTCAGTGGTGGCTCTGGCTGGAG |
| 1791 | GCTCCAGCCAGAGCCACCACTGACT | 1967 | AGTCAGTGGTGGCTCTGGCTGGAGC |
| 1792 | GGCTCCAGCCAGAGCCACCACTGAC | 1968 | GTCAGTGGTGGCTCTGGCTGGAGCC |
| 1793 | GGGCTCCAGCCAGAGCCACCACTGA | 1969 | TCAGTGGTGGCTCTGGCTGGAGCCC |
| 1794 | TGGGCTCCAGCCAGAGCCACCACTG | 1970 | CAGTGGTGGCTCTGGCTGGAGCCCA |
| 1795 | ATGGGCTCCAGCCAGAGCCACCACT | 1971 | AGTGGTGGCTCTGGCTGGAGCCCAT |
| 1796 | GATGGGCTCCAGCCAGAGCCACCAC | 1972 | GTGGTGGCTCTGGCTGGAGCCCATC |
| 1797 | TGATGGGCTCCAGCCAGAGCCACCA | 1973 | TGGTGGCTCTGGCTGGAGCCCATCA |
| 1798 | CTGATGGGCTCCAGCCAGAGCCACC | 1974 | GGTGGCTCTGGCTGGAGCCCATCAG |
| 1799 | CCTGATGGGCTCCAGCCAGAGCCAC | 1975 | GTGGCTCTGGCTGGAGCCCATCAGG |
| 1800 | ACCTGATGGGCTCCAGCCAGAGCCA | 1976 | TGGCTCTGGCTGGAGCCCATCAGGT |
| 1801 | CACCTGATGGGCTCCAGCCAGAGCC | 1977 | GGCTCTGGCTGGAGCCCATCAGGTG |
| 1802 | ACACCTGATGGGCTCCAGCCAGAGC | 1978 | GCTCTGGCTGGAGCCCATCAGGTGT |
| 1803 | CACACCTGATGGGCTCCAGCCAGAG | 1979 | CTCTGGCTGGAGCCCATCAGGTGTG |
| 1804 | TCACACCTGATGGGCTCCAGCCAGA | 1980 | TCTGGCTGGAGCCCATCAGGTGTGA |
| 1805 | TTCACACCTGATGGGCTCCAGCCAG | 1981 | CTGGCTGGAGCCCATCAGGTGTGAA |
| 1806 | GTTCACACCTGATGGGCTCCAGCCA | 1982 | TGGCTGGAGCCCATCAGGTGTGAAC |
| 1807 | CGTTCACACCTGATGGGCTCCAGCC | 1983 | GGCTGGAGCCCATCAGGTGTGAACG |
| 1808 | TCGTTCACACCTGATGGGCTCCAGC | 1984 | GCTGGAGCCCATCAGGTGTGAACGA |
| 1809 | CTCGTTCACACCTGATGGGCTCCAG | 1985 | CTGGAGCCCATCAGGTGTGAACGAG |
| 1810 | GCTCGTTCACACCTGATGGGCTCCA | 1986 | TGGAGCCCATCAGGTGTGAACGAGC |
| 1811 | GGCTCGTTCACACCTGATGGGCTCC | 1987 | GGAGCCCATCAGGTGTGAACGAGCC |
| 1812 | AGGCTCGTTCACACCTGATGGGCTC | 1988 | GAGCCCATCAGGTGTGAACGAGCCT |
| 1813 | GAGGCTCGTTCACACCTGATGGGCT | 1989 | AGCCCATCAGGTGTGAACGAGCCTC |
| 1814 | GGAGGCTCGTTCACACCTGATGGGC | 1990 | GCCCATCAGGTGTGAACGAGCCTCC |
| 1815 | GGGAGGCTCGTTCACACCTGATGGG | 1991 | CCCATCAGGTGTGAACGAGCCTCCC |
| 1816 | AGGGAGGCTCGTTCACACCTGATGG | 1992 | CCATCAGGTGTGAACGAGCCTCCCT |
| 1817 | GAGGGAGGCTCGTTCACACCTGATG | 1993 | CATCAGGTGTGAACGAGCCTCCCTC |
| 1818 | GGAGGGAGGCTCGTTCACACCTGAT | 1994 | ATCAGGTGTGAACGAGCCTCCCTCC |
| 1819 | GGGAGGGAGGCTCGTTCACACCTGA | 1995 | TCAGGTGTGAACGAGCCTCCCTCCC |
| 1820 | GGGGAGGGAGGCTCGTTCACACCTG | 1996 | CAGGTGTGAACGAGCCTCCCTCCCC |
| 1821 | AGGGGAGGGAGGCTCGTTCACACCT | 1997 | AGGTGTGAACGAGCCTCCCTCCCCT |
| 1822 | AAGGGGAGGGAGGCTCGTTCACACC | 1998 | GGTGTGAACGAGCCTCCCTCCCCTT |
| 1823 | AAAGGGGAGGGAGGCTCGTTCACAC | 1999 | GTGTGAACGAGCCTCCCTCCCCTTT |
| 1824 | GAAAGGGGAGGGAGGCTCGTTCACA | 2000 | TGTGAACGAGCCTCCCTCCCCTTTC |
| 1825 | AGAAAGGGGAGGGAGGCTCGTTCAC | 2001 | GTGAACGAGCCTCCCTCCCCTTTCT |
| 1826 | AAGAAAGGGGAGGGAGGCTCGTTCA | 2002 | TGAACGAGCCTCCCTCCCCTTTCTT |
| 1827 | GAAGAAAGGGGAGGGAGGCTCGTTC | 2003 | GAACGAGCCTCCCTCCCCTTTCTTC |
| 1828 | AGAAGAAAGGGGAGGGAGGCTCGTT | 2004 | AACGAGCCTCCCTCCCCTTTCTTCT |
| 1829 | GAGAAGAAAGGGGAGGGAGGCTCGT | 2005 | ACGAGCCTCCCTCCCCTTTCTTCTC |
| 1830 | CGAGAAGAAAGGGGAGGGAGGCTCG | 2006 | CGAGCCTCCCTCCCCTTTCTTCTCG |
| 1831 | CCGAGAAGAAAGGGGAGGGAGGCTC | 2007 | GAGCCTCCCTCCCCTTTCTTCTCGG |
| 1832 | ACCGAGAAGAAAGGGGAGGGAGGCT | 2008 | AGCCTCCCTCCCCTTTCTTCTCGGT |
| 1833 | CACCGAGAAGAAAGGGGAGGGAGGC | 2009 | GCCTCCCTCCCCTTTCTTCTCGGTG |
| 1834 | GCACCGAGAAGAAAGGGGAGGGAGG | 2010 | CCTCCCTCCCCTTTCTTCTCGGTGC |
| 1835 | AGCACCGAGAAGAAAGGGGAGGGAG | 2011 | CTCCCTCCCCTTTCTTCTCGGTGCT |
| 1836 | AAGCACCGAGAAGAAAGGGGAGGGA | 2012 | TCCCTCCCCTTTCTTCTCGGTGCTT |
| 1837 | GAAGCACCGAGAAGAAAGGGGAGGG | 2013 | CCCTCCCCTTTCTTCTCGGTGCTTC |
| 1838 | AGAAGCACCGAGAAGAAAGGGGAGG | 2014 | CCTCCCCTTTCTTCTCGGTGCTTCT |
| 1839 | AAGAAGCACCGAGAAGAAAGGGGAG | 2015 | CTCCCCTTTCTTCTCGGTGCTTCTT |
| 1840 | GAAGAAGCACCGAGAAGAAAGGGGA | 2016 | TCCCCTTTCTTCTCGGTGCTTCTTC |
| 1841 | AGAAGAAGCACCGAGAAGAAAGGGG | 2017 | CCCCTTTCTTCTCGGTGCTTCTTCT |
| 1842 | GAGAAGAAGCACCGAGAAGAAAGGG | 2018 | CCCTTTCTTCTCGGTGCTTCTTCTC |
| 1843 | CGAGAAGAAGCACCGAGAAGAAAGG | 2019 | CCTTTCTTCTCGGTGCTTCTTCTCG |
| 1844 | ACGAGAAGAAGCACCGAGAAGAAAG | 2020 | CTTTCTTCTCGGTGCTTCTTCTCGT |
| 1845 | CACGAGAAGAAGCACCGAGAAGAAA | 2021 | TTTCTTCTCGGTGCTTCTTCTCGTG |
| 1846 | TCACGAGAAGAAGCACCGAGAAGAA | 2022 | TTCTTCTCGGTGCTTCTTCTCGTGA |
| 1847 | GTCACGAGAAGAAGCACCGAGAAGA | 2023 | TCTTCTCGGTGCTTCTTCTCGTGAC |
| 1848 | AGTCACGAGAAGAAGCACCGAGAAG | 2024 | CTTCTCGGTGCTTCTTCTCGTGACT |
| 1849 | AAGTCACGAGAAGAAGCACCGAGAA | 2025 | TTCTCGGTGCTTCTTCTCGTGACTT |
| 1850 | CAAGTCACGAGAAGAAGCACCGAGA | 2026 | TCTCGGTGCTTCTTCTCGTGACTTG |
| 1851 | CCAAGTCACGAGAAGAAGCACCGAG | 2027 | CTCGGTGCTTCTTCTCGTGACTTGG |

*At least one (i.e., one or more) nucleoside linkage of the oligonucleotide sequence is independently selected from a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

TABLE 7B
Example KCNQ2 AON Sequences complementary to a sequence of a KCNQ2
transcript.

| SEQ ID | AON Sequence* | SEQ ID | Target Sequence |
|---|---|---|---|
| NO: | (5′→3′) | NO: | (5′→3′) |
| 2028 | TCACCACCACCACCACCATCATCAC | 2530 | GTGATGATGGTGGTGGTGGTGGTGA |
| 2029 | ATCACCACCACCACCACCATCATCA | 2531 | TGATGATGGTGGTGGTGGTGGTGAT |
| 2030 | CATCACCACCACCACCACCATCATC | 2532 | GATGATGGTGGTGGTGGTGGTGATG |
| 2031 | CCATCACCACCACCACCACCATCAT | 2533 | ATGATGGTGGTGGTGGTGGTGATGG |
| 2032 | ACCATCACCACCACCACCACCATCA | 2534 | TGATGGTGGTGGTGGTGGTGATGGT |
| 2033 | CACCATCACCACCACCACCACCATC | 2535 | GATGGTGGTGGTGGTGGTGATGGTG |
| 2034 | TCACCATCACCACCACCACCACCAT | 2536 | ATGGTGGTGGTGGTGGTGATGGTGA |
| 2035 | ATCACCATCACCACCACCACCACCA | 2537 | TGGTGGTGGTGGTGGTGATGGTGAT |
| 2036 | CATCACCATCACCACCACCACCACC | 2538 | GGTGGTGGTGGTGGTGATGGTGATG |
| 2037 | CCATCACCATCACCACCACCACCAC | 2539 | GTGGTGGTGGTGGTGATGGTGATGG |
| 2038 | ACCATCACCATCACCACCACCACCA | 2540 | TGGTGGTGGTGGTGATGGTGATGGT |
| 2039 | CACCATCACCATCACCACCACCACC | 2541 | GGTGGTGGTGGTGATGGTGATGGTG |
| 2040 | CCACCATCACCATCACCACCACCAC | 2542 | GTGGTGGTGGTGATGGTGATGGTGG |
| 2041 | ACCACCATCACCATCACCACCACCA | 2543 | TGGTGGTGGTGATGGTGATGGTGGT |
| 2042 | CACCACCATCACCATCACCACCACC | 2544 | GGTGGTGGTGATGGTGATGGTGGTG |
| 2043 | TCACCACCATCACCATCACCACCAC | 2545 | GTGGTGGTGATGGTGATGGTGGTGA |
| 2044 | ATCACCACCATCACCATCACCACCA | 2546 | TGGTGGTGATGGTGATGGTGGTGAT |
| 2045 | CATCACCACCATCACCATCACCACC | 2547 | GGTGGTGATGGTGATGGTGGTGATG |
| 2046 | CCATCACCACCATCACCATCACCAC | 2548 | GTGGTGATGGTGATGGTGGTGATGG |
| 2047 | ACCATCACCACCATCACCATCACCA | 2549 | TGGTGATGGTGATGGTGGTGATGGT |
| 2048 | CACCATCACCACCATCACCATCACC | 2550 | GGTGATGGTGATGGTGGTGATGGTG |
| 2049 | TCACCATCACCACCATCACCATCAC | 2551 | GTGATGGTGATGGTGGTGATGGTGA |
| 2050 | ATCACCATCACCACCATCACCATCA | 2552 | TGATGGTGATGGTGGTGATGGTGAT |
| 2051 | CATCACCATCACCACCATCACCATC | 2553 | GATGGTGATGGTGGTGATGGTGATG |
| 2052 | CCATCACCATCACCACCATCACCAT | 2554 | ATGGTGATGGTGGTGATGGTGATGG |
| 2053 | ACCATCACCATCACCACCATCACCA | 2555 | TGGTGATGGTGGTGATGGTGATGGT |
| 2054 | CACCATCACCATCACCACCATCACC | 2556 | GGTGATGGTGGTGATGGTGATGGTG |
| 2055 | CCACCATCACCATCACCACCATCAC | 2557 | GTGATGGTGGTGATGGTGATGGTGG |
| 2056 | ACCACCATCACCATCACCACCATCA | 2558 | TGATGGTGGTGATGGTGATGGTGGT |
| 2057 | CACCACCATCACCATCACCACCATC | 2559 | GATGGTGGTGATGGTGATGGTGGTG |
| 2058 | CCACCACCATCACCATCACCACCAT | 2560 | ATGGTGGTGATGGTGATGGTGGTGG |
| 2059 | ACCACCACCATCACCATCACCACCA | 2561 | TGGTGGTGATGGTGATGGTGGTGGT |
| 2060 | CACCACCACCATCACCATCACCACC | 2562 | GGTGGTGATGGTGATGGTGGTGGTG |
| 2061 | CCACCACCACCATCACCATCACCAC | 2563 | GTGGTGATGGTGATGGTGGTGGTGG |
| 2062 | ACCACCACCACCATCACCATCACCA | 2564 | TGGTGATGGTGATGGTGGTGGTGGT |
| 2063 | CACCACCACCACCATCACCATCACC | 2565 | GGTGATGGTGATGGTGGTGGTGGTG |
| 2064 | CCACCACCACCACCATCACCATCAC | 2566 | GTGATGGTGATGGTGGTGGTGGTGG |
| 2065 | ACCACCACCACCACCATCACCATCA | 2567 | TGATGGTGATGGTGGTGGTGGTGGT |
| 2066 | CACCACCACCACCACCATCACCATC | 2568 | GATGGTGATGGTGGTGGTGGTGGTG |
| 2067 | CCACCACCACCACCACCATCACCAT | 2569 | ATGGTGATGGTGGTGGTGGTGGTGG |
| 2068 | ACCACCACCACCACCACCATCACCA | 2570 | TGGTGATGGTGGTGGTGGTGGTGGT |
| 2069 | CACCACCACCACCACCACCATCACC | 2571 | GGTGATGGTGGTGGTGGTGGTGGTG |
| 2070 | CCACCACCACCACCACCACCATCAC | 2572 | GTGATGGTGGTGGTGGTGGTGGTGG |
| 2071 | ACCACCACCACCACCACCACCATCA | 2573 | TGATGGTGGTGGTGGTGGTGGTGGT |
| 2072 | CACCACCACCACCACCACCACCATC | 2574 | GATGGTGGTGGTGGTGGTGGTGGTG |
| 2073 | TCACCACCACCACCACCACCACCAT | 2575 | ATGGTGGTGGTGGTGGTGGTGGTGA |
| 2074 | ATCACCACCACCACCACCACCACCA | 2576 | TGGTGGTGGTGGTGGTGGTGGTGAT |
| 2075 | CATCACCACCACCACCACCACCACC | 2577 | GGTGGTGGTGGTGGTGGTGGTGATG |
| 2076 | CCATCACCACCACCACCACCACCAC | 2578 | GTGGTGGTGGTGGTGGTGGTGATGG |
| 2077 | ACCATCACCACCACCACCACCACCA | 2579 | TGGTGGTGGTGGTGGTGGTGATGGT |
| 2078 | CACCATCACCACCACCACCACCACC | 2580 | GGTGGTGGTGGTGGTGGTGATGGTG |
| 2079 | CCACCATCACCACCACCACCACCAC | 2581 | GTGGTGGTGGTGGTGGTGATGGTGG |
| 2080 | ACCACCATCACCACCACCACCACCA | 2582 | TGGTGGTGGTGGTGGTGATGGTGGT |
| 2081 | CACCACCATCACCACCACCACCACC | 2583 | GGTGGTGGTGGTGGTGATGGTGGTG |
| 2082 | CCACCACCATCACCACCACCACCAC | 2584 | GTGGTGGTGGTGGTGATGGTGGTGG |
| 2083 | ACCACCACCATCACCACCACCACCA | 2585 | TGGTGGTGGTGGTGATGGTGGTGGT |
| 2084 | CACCACCACCATCACCACCACCACC | 2586 | GGTGGTGGTGGTGATGGTGGTGGTG |
| 2085 | TCACCACCACCATCACCACCACCAC | 2587 | GTGGTGGTGGTGATGGTGGTGGTGA |
| 2086 | ATCACCACCACCATCACCACCACCA | 2588 | TGGTGGTGGTGATGGTGGTGGTGAT |
| 2087 | CATCACCACCACCATCACCACCACC | 2589 | GGTGGTGGTGATGGTGGTGGTGATG |
| 2088 | CCATCACCACCACCATCACCACCAC | 2590 | GTGGTGGTGATGGTGGTGGTGATGG |
| 2089 | ACCATCACCACCACCATCACCACCA | 2591 | TGGTGGTGATGGTGGTGGTGATGGT |
| 2090 | AACCATCACCACCACCATCACCACC | 2592 | GGTGGTGATGGTGGTGGTGATGGTT |
| 2091 | AAACCATCACCACCACCATCACCAC | 2593 | GTGGTGATGGTGGTGGTGATGGTTT |
| 2092 | AAAACCATCACCACCACCATCACCA | 2594 | TGGTGATGGTGGTGGTGATGGTTTT |
| 2093 | CAAAACCATCACCACCACCATCACC | 2595 | GGTGATGGTGGTGGTGATGGTTTTG |
| 2094 | CCAAAACCATCACCACCACCATCAC | 2596 | GTGATGGTGGTGGTGATGGTTTTGG |
| 2095 | ACCAAAACCATCACCACCACCATCA | 2597 | TGATGGTGGTGGTGATGGTTTTGGT |
| 2096 | CACCAAAACCATCACCACCACCATC | 2598 | GATGGTGGTGGTGATGGTTTTGGTG |
| 2097 | CCACCAAAACCATCACCACCACCAT | 2599 | ATGGTGGTGGTGATGGTTTTGGTGG |
| 2098 | ACCACCAAAACCATCACCACCACCA | 2600 | TGGTGGTGGTGATGGTTTTGGTGGT |
| 2099 | CACCACCAAAACCATCACCACCACC | 2601 | GGTGGTGGTGATGGTTTTGGTGGTG |
| 2100 | CCACCACCAAAACCATCACCACCAC | 2602 | GTGGTGGTGATGGTTTTGGTGGTGG |
| 2101 | ACCACCACCAAAACCATCACCACCA | 2603 | TGGTGGTGATGGTTTTGGTGGTGGT |
| 2102 | CACCACCACCAAAACCATCACCACC | 2604 | GGTGGTGATGGTTTTGGTGGTGGTG |
| 2103 | TCACCACCACCAAAACCATCACCAC | 2605 | GTGGTGATGGTTTTGGTGGTGGTGA |
| 2104 | ATCACCACCACCAAAACCATCACCA | 2606 | TGGTGATGGTTTTGGTGGTGGTGAT |
| 2105 | CATCACCACCACCAAAACCATCACC | 2607 | GGTGATGGTTTTGGTGGTGGTGATG |
| 2106 | CCATCACCACCACCAAAACCATCAC | 2608 | GTGATGGTTTTGGTGGTGGTGATGG |
| 2107 | ACCATCACCACCACCAAAACCATCA | 2609 | TGATGGTTTTGGTGGTGGTGATGGT |
| 2108 | CACCATCACCACCACCAAAACCATC | 2610 | GATGGTTTTGGTGGTGGTGATGGTG |
| 2109 | CCACCATCACCACCACCAAAACCAT | 2611 | ATGGTTTTGGTGGTGGTGATGGTGG |
| 2110 | ACCACCATCACCACCACCAAAACCA | 2612 | TGGTTTTGGTGGTGGTGATGGTGGT |
| 2111 | GACCACCATCACCACCACCAAAACC | 2613 | GGTTTTGGTGGTGGTGATGGTGGTC |
| 2112 | TGACCACCATCACCACCACCAAAAC | 2614 | GTTTTGGTGGTGGTGATGGTGGTCA |
| 2113 | ATGACCACCATCACCACCACCAAAA | 2615 | TTTTGGTGGTGGTGATGGTGGTCAT |
| 2114 | CATGACCACCATCACCACCACCAAA | 2616 | TTTGGTGGTGGTGATGGTGGTCATG |
| 2115 | CCATGACCACCATCACCACCACCAA | 2617 | TTGGTGGTGGTGATGGTGGTCATGG |
| 2116 | ACCATGACCACCATCACCACCACCA | 2618 | TGGTGGTGGTGATGGTGGTCATGGT |
| 2117 | CACCATGACCACCATCACCACCACC | 2619 | GGTGGTGGTGATGGTGGTCATGGTG |
| 2118 | ACACCATGACCACCATCACCACCAC | 2620 | GTGGTGGTGATGGTGGTCATGGTGT |
| 2119 | AACACCATGACCACCATCACCACCA | 2621 | TGGTGGTGATGGTGGTCATGGTGTT |
| 2120 | CAACACCATGACCACCATCACCACC | 2622 | GGTGGTGATGGTGGTCATGGTGTTG |
| 2121 | TCAACACCATGACCACCATCACCAC | 2623 | GTGGTGATGGTGGTCATGGTGTTGA |
| 2122 | ATCAACACCATGACCACCATCACCA | 2624 | TGGTGATGGTGGTCATGGTGTTGAT |
| 2123 | CATCAACACCATGACCACCATCACC | 2625 | GGTGATGGTGGTCATGGTGTTGATG |
| 2124 | ACATCAACACCATGACCACCATCAC | 2626 | GTGATGGTGGTCATGGTGTTGATGT |
| 2125 | CACATCAACACCATGACCACCATCA | 2627 | TGATGGTGGTCATGGTGTTGATGTG |
| 2126 | TCACATCAACACCATGACCACCATC | 2628 | GATGGTGGTCATGGTGTTGATGTGA |
| 2127 | ATCACATCAACACCATGACCACCAT | 2629 | ATGGTGGTCATGGTGTTGATGTGAT |
| 2128 | CATCACATCAACACCATGACCACCA | 2630 | TGGTGGTCATGGTGTTGATGTGATG |
| 2129 | CCATCACATCAACACCATGACCACC | 2631 | GGTGGTCATGGTGTTGATGTGATGG |
| 2130 | ACCATCACATCAACACCATGACCAC | 2632 | GTGGTCATGGTGTTGATGTGATGGT |
| 2131 | CACCATCACATCAACACCATGACCA | 2633 | TGGTCATGGTGTTGATGTGATGGTG |
| 2132 | CCACCATCACATCAACACCATGACC | 2634 | GGTCATGGTGTTGATGTGATGGTGG |
| 2133 | ACCACCATCACATCAACACCATGAC | 2635 | GTCATGGTGTTGATGTGATGGTGGT |
| 2134 | CACCACCATCACATCAACACCATGA | 2636 | TCATGGTGTTGATGTGATGGTGGTG |
| 2135 | CCACCACCATCACATCAACACCATG | 2637 | CATGGTGTTGATGTGATGGTGGTGG |
| 2136 | ACCACCACCATCACATCAACACCAT | 2638 | ATGGTGTTGATGTGATGGTGGTGGT |
| 2137 | CACCACCACCATCACATCAACACCA | 2639 | TGGTGTTGATGTGATGGTGGTGGTG |
| 2138 | ACACCACCACCATCACATCAACACC | 2640 | GGTGTTGATGTGATGGTGGTGGTGT |
| 2139 | AACACCACCACCATCACATCAACAC | 2641 | GTGTTGATGTGATGGTGGTGGTGTT |
| 2140 | CAACACCACCACCATCACATCAACA | 2642 | TGTTGATGTGATGGTGGTGGTGTTG |
| 2141 | CCAACACCACCACCATCACATCAAC | 2643 | GTTGATGTGATGGTGGTGGTGTTGG |
| 2142 | ACCAACACCACCACCATCACATCAA | 2644 | TTGATGTGATGGTGGTGGTGTTGGT |
| 2143 | CACCAACACCACCACCATCACATCA | 2645 | TGATGTGATGGTGGTGGTGTTGGTG |
| 2144 | CCACCAACACCACCACCATCACATC | 2646 | GATGTGATGGTGGTGGTGTTGGTGG |
| 2145 | ACCACCAACACCACCACCATCACAT | 2647 | ATGTGATGGTGGTGGTGTTGGTGGT |
| 2146 | CACCACCAACACCACCACCATCACA | 2648 | TGTGATGGTGGTGGTGTTGGTGGTG |
| 2147 | CCACCACCAACACCACCACCATCAC | 2649 | GTGATGGTGGTGGTGTTGGTGGTGG |
| 2148 | ACCACCACCAACACCACCACCATCA | 2650 | TGATGGTGGTGGTGTTGGTGGTGGT |
| 2149 | CACCACCACCAACACCACCACCATC | 2651 | GATGGTGGTGGTGTTGGTGGTGGTG |
| 2150 | TCACCACCACCAACACCACCACCAT | 2652 | ATGGTGGTGGTGTTGGTGGTGGTGA |
| 2151 | ATCACCACCACCAACACCACCACCA | 2653 | TGGTGGTGGTGTTGGTGGTGGTGAT |
| 2152 | CATCACCACCACCAACACCACCACC | 2654 | GGTGGTGGTGTTGGTGGTGGTGATG |
| 2153 | CCATCACCACCACCAACACCACCAC | 2655 | GTGGTGGTGTTGGTGGTGGTGATGG |
| 2154 | ACCATCACCACCACCAACACCACCA | 2656 | TGGTGGTGTTGGTGGTGGTGATGGT |
| 2155 | CACCATCACCACCACCAACACCACC | 2657 | GGTGGTGTTGGTGGTGGTGATGGTG |
| 2156 | TCACCATCACCACCACCAACACCAC | 2658 | GTGGTGTTGGTGGTGGTGATGGTGA |
| 2157 | ATCACCATCACCACCACCAACACCA | 2659 | TGGTGTTGGTGGTGGTGATGGTGAT |
| 2158 | CATCACCATCACCACCACCAACACC | 2660 | GGTGTTGGTGGTGGTGATGGTGATG |
| 2159 | GCATCACCATCACCACCACCAACAC | 2661 | GTGTTGGTGGTGGTGATGGTGATGC |
| 2160 | AGCATCACCATCACCACCACCAACA | 2662 | TGTTGGTGGTGGTGATGGTGATGCT |
| 2161 | AAGCATCACCATCACCACCACCAAC | 2663 | GTTGGTGGTGGTGATGGTGATGCTT |
| 2162 | GAAGCATCACCATCACCACCACCAA | 2664 | TTGGTGGTGGTGATGGTGATGCTTC |
| 2163 | AGAAGCATCACCATCACCACCACCA | 2665 | TGGTGGTGGTGATGGTGATGCTTCT |
| 2164 | CAGAAGCATCACCATCACCACCACC | 2666 | GGTGGTGGTGATGGTGATGCTTCTG |
| 2165 | CCAGAAGCATCACCATCACCACCAC | 2667 | GTGGTGGTGATGGTGATGCTTCTGG |
| 2166 | ACCAGAAGCATCACCATCACCACCA | 2668 | TGGTGGTGATGGTGATGCTTCTGGT |
| 2167 | CACCAGAAGCATCACCATCACCACC | 2669 | GGTGGTGATGGTGATGCTTCTGGTG |
| 2168 | TCACCAGAAGCATCACCATCACCAC | 2670 | GTGGTGATGGTGATGCTTCTGGTGA |
| 2169 | ATCACCAGAAGCATCACCATCACCA | 2671 | TGGTGATGGTGATGCTTCTGGTGAT |
| 2170 | CATCACCAGAAGCATCACCATCACC | 2672 | GGTGATGGTGATGCTTCTGGTGATG |
| 2171 | CCATCACCAGAAGCATCACCATCAC | 2673 | GTGATGGTGATGCTTCTGGTGATGG |
| 2172 | ACCATCACCAGAAGCATCACCATCA | 2674 | TGATGGTGATGCTTCTGGTGATGGT |
| 2173 | CACCATCACCAGAAGCATCACCATC | 2675 | GATGGTGATGCTTCTGGTGATGGTG |
| 2174 | CCACCATCACCAGAAGCATCACCAT | 2676 | ATGGTGATGCTTCTGGTGATGGTGG |
| 2175 | ACCACCATCACCAGAAGCATCACCA | 2677 | TGGTGATGCTTCTGGTGATGGTGGT |
| 2176 | GACCACCATCACCAGAAGCATCACC | 2678 | GGTGATGCTTCTGGTGATGGTGGTC |
| 2177 | TGACCACCATCACCAGAAGCATCAC | 2679 | GTGATGCTTCTGGTGATGGTGGTCA |
| 2178 | ATGACCACCATCACCAGAAGCATCA | 2680 | TGATGCTTCTGGTGATGGTGGTCAT |
| 2179 | CATGACCACCATCACCAGAAGCATC | 2681 | GATGCTTCTGGTGATGGTGGTCATG |
| 2180 | TCATGACCACCATCACCAGAAGCAT | 2682 | ATGCTTCTGGTGATGGTGGTCATGA |
| 2181 | ATCATGACCACCATCACCAGAAGCA | 2683 | TGCTTCTGGTGATGGTGGTCATGAT |
| 2182 | CATCATGACCACCATCACCAGAAGC | 2684 | GCTTCTGGTGATGGTGGTCATGATG |
| 2183 | CCATCATGACCACCATCACCAGAAG | 2685 | CTTCTGGTGATGGTGGTCATGATGG |
| 2184 | ACCATCATGACCACCATCACCAGAA | 2686 | TTCTGGTGATGGTGGTCATGATGGT |
| 2185 | CACCATCATGACCACCATCACCAGA | 2687 | TCTGGTGATGGTGGTCATGATGGTG |
| 2186 | CCACCATCATGACCACCATCACCAG | 2688 | CTGGTGATGGTGGTCATGATGGTGG |
| 2187 | ACCACCATCATGACCACCATCACCA | 2689 | TGGTGATGGTGGTCATGATGGTGGT |
| 2188 | CACCACCATCATGACCACCATCACC | 2690 | GGTGATGGTGGTCATGATGGTGGTG |
| 2189 | GCACCACCATCATGACCACCATCAC | 2691 | GTGATGGTGGTCATGATGGTGGTGC |
| 2190 | GGCACCACCATCATGACCACCATCA | 2692 | TGATGGTGGTCATGATGGTGGTGCC |
| 2191 | CGGCACCACCATCATGACCACCATC | 2693 | GATGGTGGTCATGATGGTGGTGCCG |
| 2192 | ACGGCACCACCATCATGACCACCAT | 2694 | ATGGTGGTCATGATGGTGGTGCCGT |
| 2193 | GACGGCACCACCATCATGACCACCA | 2695 | TGGTGGTCATGATGGTGGTGCCGTC |
| 2194 | AGACGGCACCACCATCATGACCACC | 2696 | GGTGGTCATGATGGTGGTGCCGTCT |
| 2195 | CAGACGGCACCACCATCATGACCAC | 2697 | GTGGTCATGATGGTGGTGCCGTCTG |
| 2196 | GCAGACGGCACCACCATCATGACCA | 2698 | TGGTCATGATGGTGGTGCCGTCTGC |
| 2197 | GGCAGACGGCACCACCATCATGACC | 2699 | GGTCATGATGGTGGTGCCGTCTGCC |
| 2198 | AGGCAGACGGCACCACCATCATGAC | 2700 | GTCATGATGGTGGTGCCGTCTGCCT |
| 2199 | GAGGCAGACGGCACCACCATCATGA | 2701 | TCATGATGGTGGTGCCGTCTGCCTC |
| 2200 | AGAGGCAGACGGCACCACCATCATG | 2702 | CATGATGGTGGTGCCGTCTGCCTCT |
| 2201 | GAGAGGCAGACGGCACCACCATCAT | 2703 | ATGATGGTGGTGCCGTCTGCCTCTC |
| 2202 | TGAGAGGCAGACGGCACCACCATCA | 2704 | TGATGGTGGTGCCGTCTGCCTCTCA |
| 2203 | CTGAGAGGCAGACGGCACCACCATC | 2705 | GATGGTGGTGCCGTCTGCCTCTCAG |
| 2204 | CCTGAGAGGCAGACGGCACCACCAT | 2706 | ATGGTGGTGCCGTCTGCCTCTCAGG |
| 2205 | TCCTGAGAGGCAGACGGCACCACCA | 2707 | TGGTGGTGCCGTCTGCCTCTCAGGA |
| 2206 | CTCCTGAGAGGCAGACGGCACCACC | 2708 | GGTGGTGCCGTCTGCCTCTCAGGAG |
| 2207 | GCTCCTGAGAGGCAGACGGCACCAC | 2709 | GTGGTGCCGTCTGCCTCTCAGGAGC |
| 2208 | AGCTCCTGAGAGGCAGACGGCACCA | 2710 | TGGTGCCGTCTGCCTCTCAGGAGCT |
| 2209 | CAGCTCCTGAGAGGCAGACGGCACC | 2711 | GGTGCCGTCTGCCTCTCAGGAGCTG |
| 2210 | CCAGCTCCTGAGAGGCAGACGGCAC | 2712 | GTGCCGTCTGCCTCTCAGGAGCTGG |
| 2211 | ACCAGCTCCTGAGAGGCAGACGGCA | 2713 | TGCCGTCTGCCTCTCAGGAGCTGGT |
| 2212 | GACCAGCTCCTGAGAGGCAGACGGC | 2714 | GCCGTCTGCCTCTCAGGAGCTGGTC |
| 2213 | TGACCAGCTCCTGAGAGGCAGACGG | 2715 | CCGTCTGCCTCTCAGGAGCTGGTCA |
| 2214 | GTGACCAGCTCCTGAGAGGCAGACG | 2716 | CGTCTGCCTCTCAGGAGCTGGTCAC |
| 2215 | AGTGACCAGCTCCTGAGAGGCAGAC | 2717 | GTCTGCCTCTCAGGAGCTGGTCACT |
| 2216 | CAGTGACCAGCTCCTGAGAGGCAGA | 2718 | TCTGCCTCTCAGGAGCTGGTCACTG |
| 2217 | GCAGTGACCAGCTCCTGAGAGGCAG | 2719 | CTGCCTCTCAGGAGCTGGTCACTGC |
| 2218 | GGCAGTGACCAGCTCCTGAGAGGCA | 2720 | TGCCTCTCAGGAGCTGGTCACTGCC |
| 2219 | AGGCAGTGACCAGCTCCTGAGAGGC | 2721 | GCCTCTCAGGAGCTGGTCACTGCCT |
| 2220 | CAGGCAGTGACCAGCTCCTGAGAGG | 2722 | CCTCTCAGGAGCTGGTCACTGCCTG |
| 2221 | CCAGGCAGTGACCAGCTCCTGAGAG | 2723 | CTCTCAGGAGCTGGTCACTGCCTGG |
| 2222 | ACCAGGCAGTGACCAGCTCCTGAGA | 2724 | TCTCAGGAGCTGGTCACTGCCTGGT |
| 2223 | TACCAGGCAGTGACCAGCTCCTGAG | 2725 | CTCAGGAGCTGGTCACTGCCTGGTA |
| 2224 | GTACCAGGCAGTGACCAGCTCCTGA | 2726 | TCAGGAGCTGGTCACTGCCTGGTAC |
| 2225 | TGTACCAGGCAGTGACCAGCTCCTG | 2727 | CAGGAGCTGGTCACTGCCTGGTACA |
| 2226 | ATGTACCAGGCAGTGACCAGCTCCT | 2728 | AGGAGCTGGTCACTGCCTGGTACAT |
| 2227 | GATGTACCAGGCAGTGACCAGCTCC | 2729 | GGAGCTGGTCACTGCCTGGTACATC |
| 2228 | CGATGTACCAGGCAGTGACCAGCTC | 2730 | GAGCTGGTCACTGCCTGGTACATCG |
| 2229 | CCGATGTACCAGGCAGTGACCAGCT | 2731 | AGCTGGTCACTGCCTGGTACATCGG |
| 2230 | GCCGATGTACCAGGCAGTGACCAGC | 2732 | GCTGGTCACTGCCTGGTACATCGGC |
| 2231 | AGCCGATGTACCAGGCAGTGACCAG | 2733 | CTGGTCACTGCCTGGTACATCGGCT |
| 2232 | AAGCCGATGTACCAGGCAGTGACCA | 2734 | TGGTCACTGCCTGGTACATCGGCTT |
| 2233 | GAAGCCGATGTACCAGGCAGTGACC | 2735 | GGTCACTGCCTGGTACATCGGCTTC |
| 2234 | GGAAGCCGATGTACCAGGCAGTGAC | 2736 | GTCACTGCCTGGTACATCGGCTTCC |
| 2235 | AGGAAGCCGATGTACCAGGCAGTGA | 2737 | TCACTGCCTGGTACATCGGCTTCCT |
| 2236 | AAGGAAGCCGATGTACCAGGCAGTG | 2738 | CACTGCCTGGTACATCGGCTTCCTT |
| 2237 | AAAGGAAGCCGATGTACCAGGCAGT | 2739 | ACTGCCTGGTACATCGGCTTCCTTT |
| 2238 | CAAAGGAAGCCGATGTACCAGGCAG | 2740 | CTGCCTGGTACATCGGCTTCCTTTG |
| 2239 | ACAAAGGAAGCCGATGTACCAGGCA | 2741 | TGCCTGGTACATCGGCTTCCTTTGT |
| 2240 | GACAAAGGAAGCCGATGTACCAGGC | 2742 | GCCTGGTACATCGGCTTCCTTTGTC |
| 2241 | AGACAAAGGAAGCCGATGTACCAGG | 2743 | CCTGGTACATCGGCTTCCTTTGTCT |
| 2242 | GAGACAAAGGAAGCCGATGTACCAG | 2744 | CTGGTACATCGGCTTCCTTTGTCTC |
| 2243 | TGAGACAAAGGAAGCCGATGTACCA | 2745 | TGGTACATCGGCTTCCTTTGTCTCA |
| 2244 | ATGAGACAAAGGAAGCCGATGTACC | 2746 | GGTACATCGGCTTCCTTTGTCTCAT |
| 2245 | GATGAGACAAAGGAAGCCGATGTAC | 2747 | GTACATCGGCTTCCTTTGTCTCATC |
| 2246 | GGATGAGACAAAGGAAGCCGATGTA | 2748 | TACATCGGCTTCCTTTGTCTCATCC |
| 2247 | AGGATGAGACAAAGGAAGCCGATGT | 2749 | ACATCGGCTTCCTTTGTCTCATCCT |
| 2248 | CAGGATGAGACAAAGGAAGCCGATG | 2750 | CATCGGCTTCCTTTGTCTCATCCTG |
| 2249 | CCAGGATGAGACAAAGGAAGCCGAT | 2751 | ATCGGCTTCCTTTGTCTCATCCTGG |
| 2250 | GCCAGGATGAGACAAAGGAAGCCGA | 2752 | TCGGCTTCCTTTGTCTCATCCTGGC |
| 2251 | GGCCAGGATGAGACAAAGGAAGCCG | 2753 | CGGCTTCCTTTGTCTCATCCTGGCC |
| 2252 | AGGCCAGGATGAGACAAAGGAAGCC | 2754 | GGCTTCCTTTGTCTCATCCTGGCCT |
| 2253 | GAGGCCAGGATGAGACAAAGGAAGC | 2755 | GCTTCCTTTGTCTCATCCTGGCCTC |
| 2254 | CGAGGCCAGGATGAGACAAAGGAAG | 2756 | CTTCCTTTGTCTCATCCTGGCCTCG |
| 2255 | ACGAGGCCAGGATGAGACAAAGGAA | 2757 | TTCCTTTGTCTCATCCTGGCCTCGT |
| 2256 | AACGAGGCCAGGATGAGACAAAGGA | 2758 | TCCTTTGTCTCATCCTGGCCTCGTT |
| 2257 | GAACGAGGCCAGGATGAGACAAAGG | 2759 | CCTTTGTCTCATCCTGGCCTCGTTC |
| 2258 | GGAACGAGGCCAGGATGAGACAAAG | 2760 | CTTTGTCTCATCCTGGCCTCGTTCC |
| 2259 | AGGAACGAGGCCAGGATGAGACAAA | 2761 | TTTGTCTCATCCTGGCCTCGTTCCT |
| 2260 | CAGGAACGAGGCCAGGATGAGACAA | 2762 | TTGTCTCATCCTGGCCTCGTTCCTG |
| 2261 | CCAGGAACGAGGCCAGGATGAGACA | 2763 | TGTCTCATCCTGGCCTCGTTCCTGG |
| 2262 | ACCAGGAACGAGGCCAGGATGAGAC | 2764 | GTCTCATCCTGGCCTCGTTCCTGGT |
| 2263 | CACCAGGAACGAGGCCAGGATGAGA | 2765 | TCTCATCCTGGCCTCGTTCCTGGTG |
| 2264 | ACACCAGGAACGAGGCCAGGATGAG | 2766 | CTCATCCTGGCCTCGTTCCTGGTGT |
| 2265 | TACACCAGGAACGAGGCCAGGATGA | 2767 | TCATCCTGGCCTCGTTCCTGGTGTA |
| 2266 | GTACACCAGGAACGAGGCCAGGATG | 2768 | CATCCTGGCCTCGTTCCTGGTGTAC |
| 2267 | AGTACACCAGGAACGAGGCCAGGAT | 2769 | ATCCTGGCCTCGTTCCTGGTGTACT |
| 2268 | AAGTACACCAGGAACGAGGCCAGGA | 2770 | TCCTGGCCTCGTTCCTGGTGTACTT |
| 2269 | CAAGTACACCAGGAACGAGGCCAGG | 2771 | CCTGGCCTCGTTCCTGGTGTACTTG |
| 2270 | CCAAGTACACCAGGAACGAGGCCAG | 2772 | CTGGCCTCGTTCCTGGTGTACTTGG |
| 2271 | GCCAAGTACACCAGGAACGAGGCCA | 2773 | TGGCCTCGTTCCTGGTGTACTTGGC |
| 2272 | TGCCAAGTACACCAGGAACGAGGCC | 2774 | GGCCTCGTTCCTGGTGTACTTGGCA |
| 2273 | CTGCCAAGTACACCAGGAACGAGGC | 2775 | GCCTCGTTCCTGGTGTACTTGGCAG |
| 2274 | TCTGCCAAGTACACCAGGAACGAGG | 2776 | CCTCGTTCCTGGTGTACTTGGCAGA |
| 2275 | CTCTGCCAAGTACACCAGGAACGAG | 2777 | CTCGTTCCTGGTGTACTTGGCAGAG |
| 2276 | TCTCTGCCAAGTACACCAGGAACGA | 2778 | TCGTTCCTGGTGTACTTGGCAGAGA |
| 2277 | TTCTCTGCCAAGTACACCAGGAACG | 2779 | CGTTCCTGGTGTACTTGGCAGAGAA |
| 2278 | CTTCTCTGCCAAGTACACCAGGAAC | 2780 | GTTCCTGGTGTACTTGGCAGAGAAG |
| 2279 | CCTTCTCTGCCAAGTACACCAGGAA | 2781 | TTCCTGGTGTACTTGGCAGAGAAGG |
| 2280 | CCCTTCTCTGCCAAGTACACCAGGA | 2782 | TCCTGGTGTACTTGGCAGAGAAGGG |
| 2281 | CCCCTTCTCTGCCAAGTACACCAGG | 2783 | CCTGGTGTACTTGGCAGAGAAGGGG |
| 2282 | CCCCCTTCTCTGCCAAGTACACCAG | 2784 | CTGGTGTACTTGGCAGAGAAGGGGG |
| 2283 | TCCCCCTTCTCTGCCAAGTACACCA | 2785 | TGGTGTACTTGGCAGAGAAGGGGGA |
| 2284 | CTCCCCCTTCTCTGCCAAGTACACC | 2786 | GGTGTACTTGGCAGAGAAGGGGGAG |
| 2285 | TCTCCCCCTTCTCTGCCAAGTACAC | 2787 | GTGTACTTGGCAGAGAAGGGGGAGA |
| 2286 | TTCTCCCCCTTCTCTGCCAAGTACA | 2788 | TGTACTTGGCAGAGAAGGGGGAGAA |
| 2287 | GTTCTCCCCCTTCTCTGCCAAGTAC | 2789 | GTACTTGGCAGAGAAGGGGGAGAAC |
| 2288 | CGTTCTCCCCCTTCTCTGCCAAGTA | 2790 | TACTTGGCAGAGAAGGGGGAGAACG |
| 2289 | TCGTTCTCCCCCTTCTCTGCCAAGT | 2791 | ACTTGGCAGAGAAGGGGGAGAACGA |
| 2290 | GTCGTTCTCCCCCTTCTCTGCCAAG | 2792 | CTTGGCAGAGAAGGGGGAGAACGAC |
| 2291 | GGTCGTTCTCCCCCTTCTCTGCCAA | 2793 | TTGGCAGAGAAGGGGGAGAACGACC |
| 2292 | TGGTCGTTCTCCCCCTTCTCTGCCA | 2794 | TGGCAGAGAAGGGGGAGAACGACCA |
| 2293 | GTGGTCGTTCTCCCCCTTCTCTGCC | 2795 | GGCAGAGAAGGGGGAGAACGACCAC |
| 2294 | AGTGGTCGTTCTCCCCCTTCTCTGC | 2796 | GCAGAGAAGGGGGAGAACGACCACT |
| 2295 | AAGTGGTCGTTCTCCCCCTTCTCTG | 2797 | CAGAGAAGGGGGAGAACGACCACTT |
| 2296 | AAAGTGGTCGTTCTCCCCCTTCTCT | 2798 | AGAGAAGGGGGAGAACGACCACTTT |
| 2297 | CAAAGTGGTCGTTCTCCCCCTTCTC | 2799 | GAGAAGGGGGAGAACGACCACTTTG |
| 2298 | TCAAAGTGGTCGTTCTCCCCCTTCT | 2800 | AGAAGGGGGAGAACGACCACTTTGA |
| 2299 | GTCAAAGTGGTCGTTCTCCCCCTTC | 2801 | GAAGGGGGAGAACGACCACTTTGAC |
| 2300 | TGTCAAAGTGGTCGTTCTCCCCCTT | 2802 | AAGGGGGAGAACGACCACTTTGACA |
| 2301 | GTGTCAAAGTGGTCGTTCTCCCCCT | 2803 | AGGGGGAGAACGACCACTTTGACAC |
| 2302 | GGTGTCAAAGTGGTCGTTCTCCCCC | 2804 | GGGGGAGAACGACCACTTTGACACC |
| 2303 | AGGTGTCAAAGTGGTCGTTCTCCCC | 2805 | GGGGAGAACGACCACTTTGACACCT |
| 2304 | TAGGTGTCAAAGTGGTCGTTCTCCC | 2806 | GGGAGAACGACCACTTTGACACCTA |
| 2305 | GTAGGTGTCAAAGTGGTCGTTCTCC | 2807 | GGAGAACGACCACTTTGACACCTAC |
| 2306 | CGTAGGTGTCAAAGTGGTCGTTCTC | 2808 | GAGAACGACCACTTTGACACCTACG |
| 2307 | GCGTAGGTGTCAAAGTGGTCGTTCT | 2809 | AGAACGACCACTTTGACACCTACGC |
| 2308 | CGCGTAGGTGTCAAAGTGGTCGTTC | 2810 | GAACGACCACTTTGACACCTACGCG |
| 2309 | CCGCGTAGGTGTCAAAGTGGTCGTT | 2811 | AACGACCACTTTGACACCTACGCGG |
| 2310 | TCCGCGTAGGTGTCAAAGTGGTCGT | 2812 | ACGACCACTTTGACACCTACGCGGA |
| 2311 | ATCCGCGTAGGTGTCAAAGTGGTCG | 2813 | CGACCACTTTGACACCTACGCGGAT |
| 2312 | CATCCGCGTAGGTGTCAAAGTGGTC | 2814 | GACCACTTTGACACCTACGCGGATG |
| 2313 | GCATCCGCGTAGGTGTCAAAGTGGT | 2815 | ACCACTTTGACACCTACGCGGATGC |
| 2314 | TGCATCCGCGTAGGTGTCAAAGTGG | 2816 | CCACTTTGACACCTACGCGGATGCA |
| 2315 | GTGCATCCGCGTAGGTGTCAAAGTG | 2817 | CACTTTGACACCTACGCGGATGCAC |
| 2316 | AGTGCATCCGCGTAGGTGTCAAAGT | 2818 | ACTTTGACACCTACGCGGATGCACT |
| 2317 | GAGTGCATCCGCGTAGGTGTCAAAG | 2819 | CTTTGACACCTACGCGGATGCACTC |
| 2318 | AGAGTGCATCCGCGTAGGTGTCAAA | 2820 | TTTGACACCTACGCGGATGCACTCT |
| 2319 | CAGAGTGCATCCGCGTAGGTGTCAA | 2821 | TTGACACCTACGCGGATGCACTCTG |
| 2320 | CCAGAGTGCATCCGCGTAGGTGTCA | 2822 | TGACACCTACGCGGATGCACTCTGG |
| 2321 | ACCAGAGTGCATCCGCGTAGGTGTC | 2823 | GACACCTACGCGGATGCACTCTGGT |
| 2322 | CACCAGAGTGCATCCGCGTAGGTGT | 2824 | ACACCTACGCGGATGCACTCTGGTG |
| 2323 | CCACCAGAGTGCATCCGCGTAGGTG | 2825 | CACCTACGCGGATGCACTCTGGTGG |
| 2324 | CCCACCAGAGTGCATCCGCGTAGGT | 2826 | ACCTACGCGGATGCACTCTGGTGGG |
| 2325 | CCCCACCAGAGTGCATCCGCGTAGG | 2827 | CCTACGCGGATGCACTCTGGTGGGG |
| 2326 | GCCCCACCAGAGTGCATCCGCGTAG | 2828 | CTACGCGGATGCACTCTGGTGGGGC |
| 2327 | GGCCCCACCAGAGTGCATCCGCGTA | 2829 | TACGCGGATGCACTCTGGTGGGGCC |
| 2328 | AGGCCCCACCAGAGTGCATCCGCGT | 2830 | ACGCGGATGCACTCTGGTGGGGCCT |
| 2329 | CAGGCCCCACCAGAGTGCATCCGCG | 2831 | CGCGGATGCACTCTGGTGGGGCCTG |
| 2330 | CCAGGCCCCACCAGAGTGCATCCGC | 2832 | GCGGATGCACTCTGGTGGGGCCTGG |
| 2331 | ACCAGGCCCCACCAGAGTGCATCCG | 2833 | CGGATGCACTCTGGTGGGGCCTGGT |
| 2332 | CACCAGGCCCCACCAGAGTGCATCC | 2834 | GGATGCACTCTGGTGGGGCCTGGTG |
| 2333 | TCACCAGGCCCCACCAGAGTGCATC | 2835 | GATGCACTCTGGTGGGGCCTGGTGA |
| 2334 | CTCACCAGGCCCCACCAGAGTGCAT | 2836 | ATGCACTCTGGTGGGGCCTGGTGAG |
| 2335 | ACTCACCAGGCCCCACCAGAGTGCA | 2837 | TGCACTCTGGTGGGGCCTGGTGAGT |
| 2336 | AACTCACCAGGCCCCACCAGAGTGC | 2838 | GCACTCTGGTGGGGCCTGGTGAGTT |
| 2337 | CAACTCACCAGGCCCCACCAGAGTG | 2839 | CACTCTGGTGGGGCCTGGTGAGTTG |
| 2338 | ACAACTCACCAGGCCCCACCAGAGT | 2840 | ACTCTGGTGGGGCCTGGTGAGTTGT |
| 2339 | CACAACTCACCAGGCCCCACCAGAG | 2841 | CTCTGGTGGGGCCTGGTGAGTTGTG |
| 2340 | CCACAACTCACCAGGCCCCACCAGA | 2842 | TCTGGTGGGGCCTGGTGAGTTGTGG |
| 2341 | ACCACAACTCACCAGGCCCCACCAG | 2843 | CTGGTGGGGCCTGGTGAGTTGTGGT |
| 2342 | GACCACAACTCACCAGGCCCCACCA | 2844 | TGGTGGGGCCTGGTGAGTTGTGGTC |
| 2343 | TGACCACAACTCACCAGGCCCCACC | 2845 | GGTGGGGCCTGGTGAGTTGTGGTCA |
| 2344 | ATGACCACAACTCACCAGGCCCCAC | 2846 | GTGGGGCCTGGTGAGTTGTGGTCAT |
| 2345 | AATGACCACAACTCACCAGGCCCCA | 2847 | TGGGGCCTGGTGAGTTGTGGTCATT |
| 2346 | CAATGACCACAACTCACCAGGCCCC | 2848 | GGGGCCTGGTGAGTTGTGGTCATTG |
| 2347 | ACAATGACCACAACTCACCAGGCCC | 2849 | GGGCCTGGTGAGTTGTGGTCATTGT |
| 2348 | CACAATGACCACAACTCACCAGGCC | 2850 | GGCCTGGTGAGTTGTGGTCATTGTG |
| 2349 | CCACAATGACCACAACTCACCAGGC | 2851 | GCCTGGTGAGTTGTGGTCATTGTGG |
| 2350 | ACCACAATGACCACAACTCACCAGG | 2852 | CCTGGTGAGTTGTGGTCATTGTGGT |
| 2351 | AACCACAATGACCACAACTCACCAG | 2853 | CTGGTGAGTTGTGGTCATTGTGGTT |
| 2352 | AAACCACAATGACCACAACTCACCA | 2854 | TGGTGAGTTGTGGTCATTGTGGTTT |
| 2353 | AAAACCACAATGACCACAACTCACC | 2855 | GGTGAGTTGTGGTCATTGTGGTTTT |
| 2354 | GAAAACCACAATGACCACAACTCAC | 2856 | GTGAGTTGTGGTCATTGTGGTTTTC |
| 2355 | GGAAAACCACAATGACCACAACTCA | 2857 | TGAGTTGTGGTCATTGTGGTTTTCC |
| 2356 | GGGAAAACCACAATGACCACAACTC | 2858 | GAGTTGTGGTCATTGTGGTTTTCCC |
| 2357 | AGGGAAAACCACAATGACCACAACT | 2859 | AGTTGTGGTCATTGTGGTTTTCCCT |
| 2358 | AAGGGAAAACCACAATGACCACAAC | 2860 | GTTGTGGTCATTGTGGTTTTCCCTT |
| 2359 | AAAGGGAAAACCACAATGACCACAA | 2861 | TTGTGGTCATTGTGGTTTTCCCTTT |
| 2360 | GAAAGGGAAAACCACAATGACCACA | 2862 | TGTGGTCATTGTGGTTTTCCCTTTC |
| 2361 | GGAAAGGGAAAACCACAATGACCAC | 2863 | GTGGTCATTGTGGTTTTCCCTTTCC |
| 2362 | GGGAAAGGGAAAACCACAATGACCA | 2864 | TGGTCATTGTGGTTTTCCCTTTCCC |
| 2363 | AGGGAAAGGGAAAACCACAATGACC | 2865 | GGTCATTGTGGTTTTCCCTTTCCCT |
| 2364 | CAGGGAAAGGGAAAACCACAATGAC | 2866 | GTCATTGTGGTTTTCCCTTTCCCTG |
| 2365 | GCAGGGAAAGGGAAAACCACAATGA | 2867 | TCATTGTGGTTTTCCCTTTCCCTGC |
| 2366 | AGCAGGGAAAGGGAAAACCACAATG | 2868 | CATTGTGGTTTTCCCTTTCCCTGCT |
| 2367 | CAGCAGGGAAAGGGAAAACCACAAT | 2869 | ATTGTGGTTTTCCCTTTCCCTGCTG |
| 2368 | TCAGCAGGGAAAGGGAAAACCACAA | 2870 | TTGTGGTTTTCCCTTTCCCTGCTGA |
| 2369 | ATCAGCAGGGAAAGGGAAAACCACA | 2871 | TGTGGTTTTCCCTTTCCCTGCTGAT |
| 2370 | TATCAGCAGGGAAAGGGAAAACCAC | 2872 | GTGGTTTTCCCTTTCCCTGCTGATA |
| 2371 | GTATCAGCAGGGAAAGGGAAAACCA | 2873 | TGGTTTTCCCTTTCCCTGCTGATAC |
| 2372 | TGTATCAGCAGGGAAAGGGAAAACC | 2874 | GGTTTTCCCTTTCCCTGCTGATACA |
| 2373 | GTGTATCAGCAGGGAAAGGGAAAAC | 2875 | GTTTTCCCTTTCCCTGCTGATACAC |
| 2374 | GGTGTATCAGCAGGGAAAGGGAAAA | 2876 | TTTTCCCTTTCCCTGCTGATACACC |
| 2375 | GGGTGTATCAGCAGGGAAAGGGAAA | 2877 | TTTCCCTTTCCCTGCTGATACACCC |
| 2376 | GGGGTGTATCAGCAGGGAAAGGGAA | 2878 | TTCCCTTTCCCTGCTGATACACCCC |
| 2377 | AGGGGTGTATCAGCAGGGAAAGGGA | 2879 | TCCCTTTCCCTGCTGATACACCCCT |
| 2378 | CAGGGGTGTATCAGCAGGGAAAGGG | 2880 | CCCTTTCCCTGCTGATACACCCCTG |
| 2379 | ACAGGGGTGTATCAGCAGGGAAAGG | 2881 | CCTTTCCCTGCTGATACACCCCTGT |
| 2380 | GACAGGGGTGTATCAGCAGGGAAAG | 2882 | CTTTCCCTGCTGATACACCCCTGTC |
| 2381 | GGACAGGGGTGTATCAGCAGGGAAA | 2883 | TTTCCCTGCTGATACACCCCTGTCC |
| 2382 | GGGACAGGGGTGTATCAGCAGGGAA | 2884 | TTCCCTGCTGATACACCCCTGTCCC |
| 2383 | AGGGACAGGGGTGTATCAGCAGGGA | 2885 | TCCCTGCTGATACACCCCTGTCCCT |
| 2384 | CAGGGACAGGGGTGTATCAGCAGGG | 2886 | CCCTGCTGATACACCCCTGTCCCTG |
| 2385 | ACAGGGACAGGGGTGTATCAGCAGG | 2887 | CCTGCTGATACACCCCTGTCCCTGT |
| 2386 | CACAGGGACAGGGGTGTATCAGCAG | 2888 | CTGCTGATACACCCCTGTCCCTGTG |
| 2387 | GCACAGGGACAGGGGTGTATCAGCA | 2889 | TGCTGATACACCCCTGTCCCTGTGC |
| 2388 | AGCACAGGGACAGGGGTGTATCAGC | 2890 | GCTGATACACCCCTGTCCCTGTGCT |
| 2389 | CAGCACAGGGACAGGGGTGTATCAG | 2891 | CTGATACACCCCTGTCCCTGTGCTG |
| 2390 | CCAGCACAGGGACAGGGGTGTATCA | 2892 | TGATACACCCCTGTCCCTGTGCTGG |
| 2391 | CCCAGCACAGGGACAGGGGTGTATC | 2893 | GATACACCCCTGTCCCTGTGCTGGG |
| 2392 | TCCCAGCACAGGGACAGGGGTGTAT | 2894 | ATACACCCCTGTCCCTGTGCTGGGA |
| 2393 | GTCCCAGCACAGGGACAGGGGTGTA | 2895 | TACACCCCTGTCCCTGTGCTGGGAC |
| 2394 | GGTCCCAGCACAGGGACAGGGGTGT | 2896 | ACACCCCTGTCCCTGTGCTGGGACC |
| 2395 | TGGTCCCAGCACAGGGACAGGGGTG | 2897 | CACCCCTGTCCCTGTGCTGGGACCA |
| 2396 | CTGGTCCCAGCACAGGGACAGGGGT | 2898 | ACCCCTGTCCCTGTGCTGGGACCAG |
| 2397 | CCTGGTCCCAGCACAGGGACAGGGG | 2899 | CCCCTGTCCCTGTGCTGGGACCAGG |
| 2398 | GCCTGGTCCCAGCACAGGGACAGGG | 2900 | CCCTGTCCCTGTGCTGGGACCAGGC |
| 2399 | AGCCTGGTCCCAGCACAGGGACAGG | 2901 | CCTGTCCCTGTGCTGGGACCAGGCT |
| 2400 | GAGCCTGGTCCCAGCACAGGGACAG | 2902 | CTGTCCCTGTGCTGGGACCAGGCTC |
| 2401 | AGAGCCTGGTCCCAGCACAGGGACA | 2903 | TGTCCCTGTGCTGGGACCAGGCTCT |
| 2402 | GAGAGCCTGGTCCCAGCACAGGGAC | 2904 | GTCCCTGTGCTGGGACCAGGCTCTC |
| 2403 | TGAGAGCCTGGTCCCAGCACAGGGA | 2905 | TCCCTGTGCTGGGACCAGGCTCTCA |
| 2404 | GTGAGAGCCTGGTCCCAGCACAGGG | 2906 | CCCTGTGCTGGGACCAGGCTCTCAC |
| 2405 | AGTGAGAGCCTGGTCCCAGCACAGG | 2907 | CCTGTGCTGGGACCAGGCTCTCACT |
| 2406 | CAGTGAGAGCCTGGTCCCAGCACAG | 2908 | CTGTGCTGGGACCAGGCTCTCACTG |
| 2407 | CCAGTGAGAGCCTGGTCCCAGCACA | 2909 | TGTGCTGGGACCAGGCTCTCACTGG |
| 2408 | GCCAGTGAGAGCCTGGTCCCAGCAC | 2910 | GTGCTGGGACCAGGCTCTCACTGGC |
| 2409 | AGCCAGTGAGAGCCTGGTCCCAGCA | 2911 | TGCTGGGACCAGGCTCTCACTGGCT |
| 2410 | CAGCCAGTGAGAGCCTGGTCCCAGC | 2912 | GCTGGGACCAGGCTCTCACTGGCTG |
| 2411 | TCAGCCAGTGAGAGCCTGGTCCCAG | 2913 | CTGGGACCAGGCTCTCACTGGCTGA |
| 2412 | CTCAGCCAGTGAGAGCCTGGTCCCA | 2914 | TGGGACCAGGCTCTCACTGGCTGAG |
| 2413 | GCTCAGCCAGTGAGAGCCTGGTCCC | 2915 | GGGACCAGGCTCTCACTGGCTGAGC |
| 2414 | GGCTCAGCCAGTGAGAGCCTGGTCC | 2916 | GGACCAGGCTCTCACTGGCTGAGCC |
| 2415 | AGGCTCAGCCAGTGAGAGCCTGGTC | 2917 | GACCAGGCTCTCACTGGCTGAGCCT |
| 2416 | CAGGCTCAGCCAGTGAGAGCCTGGT | 2918 | ACCAGGCTCTCACTGGCTGAGCCTG |
| 2417 | GCAGGCTCAGCCAGTGAGAGCCTGG | 2919 | CCAGGCTCTCACTGGCTGAGCCTGC |
| 2418 | AGCAGGCTCAGCCAGTGAGAGCCTG | 2920 | CAGGCTCTCACTGGCTGAGCCTGCT |
| 2419 | GAGCAGGCTCAGCCAGTGAGAGCCT | 2921 | AGGCTCTCACTGGCTGAGCCTGCTC |
| 2420 | GGAGCAGGCTCAGCCAGTGAGAGCC | 2922 | GGCTCTCACTGGCTGAGCCTGCTCC |
| 2421 | TGGAGCAGGCTCAGCCAGTGAGAGC | 2923 | GCTCTCACTGGCTGAGCCTGCTCCA |
| 2422 | ATGGAGCAGGCTCAGCCAGTGAGAG | 2924 | CTCTCACTGGCTGAGCCTGCTCCAT |
| 2423 | TATGGAGCAGGCTCAGCCAGTGAGA | 2925 | TCTCACTGGCTGAGCCTGCTCCATA |
| 2424 | GTATGGAGCAGGCTCAGCCAGTGAG | 2926 | CTCACTGGCTGAGCCTGCTCCATAC |
| 2425 | TGTATGGAGCAGGCTCAGCCAGTGA | 2927 | TCACTGGCTGAGCCTGCTCCATACA |
| 2426 | ATGTATGGAGCAGGCTCAGCCAGTG | 2928 | CACTGGCTGAGCCTGCTCCATACAT |
| 2427 | GATGTATGGAGCAGGCTCAGCCAGT | 2929 | ACTGGCTGAGCCTGCTCCATACATC |
| 2428 | AGATGTATGGAGCAGGCTCAGCCAG | 2930 | CTGGCTGAGCCTGCTCCATACATCT |
| 2429 | GAGATGTATGGAGCAGGCTCAGCCA | 2931 | TGGCTGAGCCTGCTCCATACATCTC |
| 2430 | AGAGATGTATGGAGCAGGCTCAGCC | 2932 | GGCTGAGCCTGCTCCATACATCTCT |
| 2431 | AAGAGATGTATGGAGCAGGCTCAGC | 2933 | GCTGAGCCTGCTCCATACATCTCTT |
| 2432 | AAAGAGATGTATGGAGCAGGCTCAG | 2934 | CTGAGCCTGCTCCATACATCTCTTT |
| 2433 | CAAAGAGATGTATGGAGCAGGCTCA | 2935 | TGAGCCTGCTCCATACATCTCTTTG |
| 2434 | CCAAAGAGATGTATGGAGCAGGCTC | 2936 | GAGCCTGCTCCATACATCTCTTTGG |
| 2435 | CCCAAAGAGATGTATGGAGCAGGCT | 2937 | AGCCTGCTCCATACATCTCTTTGGG |
| 2436 | CCCCAAAGAGATGTATGGAGCAGGC | 2938 | GCCTGCTCCATACATCTCTTTGGGG |
| 2437 | GCCCCAAAGAGATGTATGGAGCAGG | 2939 | CCTGCTCCATACATCTCTTTGGGGC |
| 2438 | GGCCCCAAAGAGATGTATGGAGCAG | 2940 | CTGCTCCATACATCTCTTTGGGGCC |
| 2439 | TGGCCCCAAAGAGATGTATGGAGCA | 2941 | TGCTCCATACATCTCTTTGGGGCCA |
| 2440 | GTGGCCCCAAAGAGATGTATGGAGC | 2942 | GCTCCATACATCTCTTTGGGGCCAC |
| 2441 | GGTGGCCCCAAAGAGATGTATGGAG | 2943 | CTCCATACATCTCTTTGGGGCCACC |
| 2442 | AGGTGGCCCCAAAGAGATGTATGGA | 2944 | TCCATACATCTCTTTGGGGCCACCT |
| 2443 | CAGGTGGCCCCAAAGAGATGTATGG | 2945 | CCATACATCTCTTTGGGGCCACCTG |
| 2444 | GCAGGTGGCCCCAAAGAGATGTATG | 2946 | CATACATCTCTTTGGGGCCACCTGC |
| 2445 | AGCAGGTGGCCCCAAAGAGATGTAT | 2947 | ATACATCTCTTTGGGGCCACCTGCT |
| 2446 | CAGCAGGTGGCCCCAAAGAGATGTA | 2948 | TACATCTCTTTGGGGCCACCTGCTG |
| 2447 | CCAGCAGGTGGCCCCAAAGAGATGT | 2949 | ACATCTCTTTGGGGCCACCTGCTGG |
| 2448 | GCCAGCAGGTGGCCCCAAAGAGATG | 2950 | CATCTCTTTGGGGCCACCTGCTGGC |
| 2449 | AGCCAGCAGGTGGCCCCAAAGAGAT | 2951 | ATCTCTTTGGGGCCACCTGCTGGCT |
| 2450 | CAGCCAGCAGGTGGCCCCAAAGAGA | 2952 | TCTCTTTGGGGCCACCTGCTGGCTG |
| 2451 | GCAGCCAGCAGGTGGCCCCAAAGAG | 2953 | CTCTTTGGGGCCACCTGCTGGCTGC |
| 2452 | GGCAGCCAGCAGGTGGCCCCAAAGA | 2954 | TCTTTGGGGCCACCTGCTGGCTGCC |
| 2453 | GGGCAGCCAGCAGGTGGCCCCAAAG | 2955 | CTTTGGGGCCACCTGCTGGCTGCCC |
| 2454 | CGGGCAGCCAGCAGGTGGCCCCAAA | 2956 | TTTGGGGCCACCTGCTGGCTGCCCG |
| 2455 | ACGGGCAGCCAGCAGGTGGCCCCAA | 2957 | TTGGGGCCACCTGCTGGCTGCCCGT |
| 2456 | CACGGGCAGCCAGCAGGTGGCCCCA | 2958 | TGGGGCCACCTGCTGGCTGCCCGTG |
| 2457 | CCACGGGCAGCCAGCAGGTGGCCCC | 2959 | GGGGCCACCTGCTGGCTGCCCGTGG |
| 2458 | ACCACGGGCAGCCAGCAGGTGGCCC | 2960 | GGGCCACCTGCTGGCTGCCCGTGGT |
| 2459 | GACCACGGGCAGCCAGCAGGTGGCC | 2961 | GGCCACCTGCTGGCTGCCCGTGGTC |
| 2460 | TGACCACGGGCAGCCAGCAGGTGGC | 2962 | GCCACCTGCTGGCTGCCCGTGGTCA |
| 2461 | ATGACCACGGGCAGCCAGCAGGTGG | 2963 | CCACCTGCTGGCTGCCCGTGGTCAT |
| 2462 | CATGACCACGGGCAGCCAGCAGGTG | 2964 | CACCTGCTGGCTGCCCGTGGTCATG |
| 2463 | TCATGACCACGGGCAGCCAGCAGGT | 2965 | ACCTGCTGGCTGCCCGTGGTCATGA |
| 2464 | ATCATGACCACGGGCAGCCAGCAGG | 2966 | CCTGCTGGCTGCCCGTGGTCATGAT |
| 2465 | CATCATGACCACGGGCAGCCAGCAG | 2967 | CTGCTGGCTGCCCGTGGTCATGATG |
| 2466 | CCATCATGACCACGGGCAGCCAGCA | 2968 | TGCTGGCTGCCCGTGGTCATGATGG |
| 2467 | GCCATCATGACCACGGGCAGCCAGC | 2969 | GCTGGCTGCCCGTGGTCATGATGGC |
| 2468 | AGCCATCATGACCACGGGCAGCCAG | 2970 | CTGGCTGCCCGTGGTCATGATGGCT |
| 2469 | AAGCCATCATGACCACGGGCAGCCA | 2971 | TGGCTGCCCGTGGTCATGATGGCTT |
| 2470 | CAAGCCATCATGACCACGGGCAGCC | 2972 | GGCTGCCCGTGGTCATGATGGCTTG |
| 2471 | ACAAGCCATCATGACCACGGGCAGC | 2973 | GCTGCCCGTGGTCATGATGGCTTGT |
| 2472 | CACAAGCCATCATGACCACGGGCAG | 2974 | CTGCCCGTGGTCATGATGGCTTGTG |
| 2473 | CCACAAGCCATCATGACCACGGGCA | 2975 | TGCCCGTGGTCATGATGGCTTGTGG |
| 2474 | ACCACAAGCCATCATGACCACGGGC | 2976 | GCCCGTGGTCATGATGGCTTGTGGT |
| 2475 | GACCACAAGCCATCATGACCACGGG | 2977 | CCCGTGGTCATGATGGCTTGTGGTC |
| 2476 | CGACCACAAGCCATCATGACCACGG | 2978 | CCGTGGTCATGATGGCTTGTGGTCG |
| 2477 | TCGACCACAAGCCATCATGACCACG | 2979 | CGTGGTCATGATGGCTTGTGGTCGA |
| 2478 | CTCGACCACAAGCCATCATGACCAC | 2980 | GTGGTCATGATGGCTTGTGGTCGAG |
| 2479 | CCTCGACCACAAGCCATCATGACCA | 2981 | TGGTCATGATGGCTTGTGGTCGAGG |
| 2480 | GCCTCGACCACAAGCCATCATGACC | 2982 | GGTCATGATGGCTTGTGGTCGAGGC |
| 2481 | CGCCTCGACCACAAGCCATCATGAC | 2983 | GTCATGATGGCTTGTGGTCGAGGCG |
| 2482 | CCGCCTCGACCACAAGCCATCATGA | 2984 | TCATGATGGCTTGTGGTCGAGGCGG |
| 2483 | CCCGCCTCGACCACAAGCCATCATG | 2985 | CATGATGGCTTGTGGTCGAGGCGGG |
| 2484 | CCCCGCCTCGACCACAAGCCATCAT | 2986 | ATGATGGCTTGTGGTCGAGGCGGGG |
| 2485 | ACCCCGCCTCGACCACAAGCCATCA | 2987 | TGATGGCTTGTGGTCGAGGCGGGGT |
| 2486 | CACCCCGCCTCGACCACAAGCCATC | 2988 | GATGGCTTGTGGTCGAGGCGGGGTG |
| 2487 | CCACCCCGCCTCGACCACAAGCCAT | 2989 | ATGGCTTGTGGTCGAGGCGGGGTGG |
| 2488 | ACCACCCCGCCTCGACCACAAGCCA | 2990 | TGGCTTGTGGTCGAGGCGGGGTGGT |
| 2489 | CACCACCCCGCCTCGACCACAAGCC | 2991 | GGCTTGTGGTCGAGGCGGGGTGGTG |
| 2490 | CCACCACCCCGCCTCGACCACAAGC | 2992 | GCTTGTGGTCGAGGCGGGGTGGTGG |
| 2491 | ACCACCACCCCGCCTCGACCACAAG | 2993 | CTTGTGGTCGAGGCGGGGTGGTGGT |
| 2492 | AACCACCACCCCGCCTCGACCACAA | 2994 | TTGTGGTCGAGGCGGGGTGGTGGTT |
| 2493 | CAACCACCACCCCGCCTCGACCACA | 2995 | TGTGGTCGAGGCGGGGTGGTGGTTG |
| 2494 | GCAACCACCACCCCGCCTCGACCAC | 2996 | GTGGTCGAGGCGGGGTGGTGGTTGC |
| 2495 | GGCAACCACCACCCCGCCTCGACCA | 2997 | TGGTCGAGGCGGGGTGGTGGTTGCC |
| 2496 | TGGCAACCACCACCCCGCCTCGACC | 2998 | GGTCGAGGCGGGGTGGTGGTTGCCA |
| 2497 | TTGGCAACCACCACCCCGCCTCGAC | 2999 | GTCGAGGCGGGGTGGTGGTTGCCAA |
| 2498 | CTTGGCAACCACCACCCCGCCTCGA | 3000 | TCGAGGCGGGGTGGTGGTTGCCAAG |
| 2499 | GCTTGGCAACCACCACCCCGCCTCG | 3001 | CGAGGCGGGGTGGTGGTTGCCAAGC |
| 2500 | AGCTTGGCAACCACCACCCCGCCTC | 3002 | GAGGCGGGGTGGTGGTTGCCAAGCT |
| 2501 | CAGCTTGGCAACCACCACCCCGCCT | 3003 | AGGCGGGGTGGTGGTTGCCAAGCTG |
| 2502 | CCAGCTTGGCAACCACCACCCCGCC | 3004 | GGCGGGGTGGTGGTTGCCAAGCTGG |
| 2503 | GCCAGCTTGGCAACCACCACCCCGC | 3005 | GCGGGGTGGTGGTTGCCAAGCTGGC |
| 2504 | CGCCAGCTTGGCAACCACCACCCCG | 3006 | CGGGGTGGTGGTTGCCAAGCTGGCG |
| 2505 | CCGCCAGCTTGGCAACCACCACCCC | 3007 | GGGGTGGTGGTTGCCAAGCTGGCGG |
| 2506 | CCCGCCAGCTTGGCAACCACCACCC | 3008 | GGGTGGTGGTTGCCAAGCTGGCGGG |
| 2507 | CCCCGCCAGCTTGGCAACCACCACC | 3009 | GGTGGTGGTTGCCAAGCTGGCGGGG |
| 2508 | CCCCCGCCAGCTTGGCAACCACCAC | 3010 | GTGGTGGTTGCCAAGCTGGCGGGGG |
| 2509 | TCCCCCGCCAGCTTGGCAACCACCA | 3011 | TGGTGGTTGCCAAGCTGGCGGGGGA |
| 2510 | CTCCCCCGCCAGCTTGGCAACCACC | 3012 | GGTGGTTGCCAAGCTGGCGGGGGAG |
| 2511 | ACTCCCCCGCCAGCTTGGCAACCAC | 3013 | GTGGTTGCCAAGCTGGCGGGGGAGT |
| 2512 | CACTCCCCCGCCAGCTTGGCAACCA | 3014 | TGGTTGCCAAGCTGGCGGGGGAGTG |
| 2513 | CCACTCCCCCGCCAGCTTGGCAACC | 3015 | GGTTGCCAAGCTGGCGGGGGAGTGG |
| 2514 | CCCACTCCCCCGCCAGCTTGGCAAC | 3016 | GTTGCCAAGCTGGCGGGGGAGTGGG |
| 2515 | ACCCACTCCCCCGCCAGCTTGGCAA | 3017 | TTGCCAAGCTGGCGGGGGAGTGGGT |
| 2516 | CACCCACTCCCCCGCCAGCTTGGCA | 3018 | TGCCAAGCTGGCGGGGGAGTGGGTG |
| 2517 | CCACCCACTCCCCCGCCAGCTTGGC | 3019 | GCCAAGCTGGCGGGGGAGTGGGTGG |
| 2518 | CCCACCCACTCCCCCGCCAGCTTGG | 3020 | CCAAGCTGGCGGGGGAGTGGGTGGG |
| 2519 | CCCCACCCACTCCCCCGCCAGCTTG | 3021 | CAAGCTGGCGGGGGAGTGGGTGGGG |
| 2520 | TCCCCACCCACTCCCCCGCCAGCTT | 3022 | AAGCTGGCGGGGGAGTGGGTGGGGA |
| 2521 | CTCCCCACCCACTCCCCCGCCAGCT | 3023 | AGCTGGCGGGGGAGTGGGTGGGGAG |
| 2522 | CCTCCCCACCCACTCCCCCGCCAGC | 3024 | GCTGGCGGGGGAGTGGGTGGGGAGG |
| 2523 | CCCTCCCCACCCACTCCCCCGCCAG | 3025 | CTGGCGGGGGAGTGGGTGGGGAGGG |
| 2524 | GCCCTCCCCACCCACTCCCCCGCCA | 3026 | TGGCGGGGGAGTGGGTGGGGAGGGC |
| 2525 | AGCCCTCCCCACCCACTCCCCCGCC | 3027 | GGCGGGGGAGTGGGTGGGGAGGGCT |
| 2526 | CAGCCCTCCCCACCCACTCCCCCGC | 3028 | GCGGGGGAGTGGGTGGGGAGGGCTG |
| 2527 | CCAGCCCTCCCCACCCACTCCCCCG | 3029 | CGGGGGAGTGGGTGGGGAGGGCTGG |
| 2528 | CCCAGCCCTCCCCACCCACTCCCCC | 3030 | GGGGGAGTGGGTGGGGAGGGCTGGG |
| 2529 | GCCCAGCCCTCCCCACCCACTCCCC | 3031 | GGGGAGTGGGTGGGGAGGGCTGGGC |

*At least one (i.e., one or more) nucleoside linkage of the oligonucleotide sequence is independently selected from a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

### KCNQ2 Transcript

In various embodiments, a KCNQ2 transcript comprises the sequence provided as SEQ ID NO: 3032.

(SEQ ID NO: 3032
AGGCGCCGAGGTGCGCGCGGAGCGAGGTGGCCGCAGCGTCTCCGCGCGCGGCCCAA
GCCCGGCAGGAGTGCGGAACCGCCGCCTCGGCCATGCGGCTCCCGGCCGGGGGGCC
TGGGCTGGGGCCCGCGCCGCCCCCCGCGCTCCGCCCCCGCTGAGCCTGAGCCCGACC
CGGGGCGCCTCCCGCCAGGCACCATGGTGCAGAAGTCGCGCAACGGCGGCGTATAC
CCCGGCCCGAGCGGGGAGAAGAAGCTGAAGGTGGGCTTCGTGGGGCTGGACCCCGG
CGCGCCCGACTCCACCCGGGACGGGGCGCTGCTGATCGCCGGCTCCGAGGCCCCCA
AGCGCGGCAGCATCCTCAGCAAACCTCGCGCGGGCGGCGCGGGCGCCGGGAAGCCC
CCCAAGCGCAACGCCTTCTACCGCAAGCTGCAGAATTTCCTCTACAACGTGCTGGAG
CGGCCGCGCGGCTGGGCGTTCATCTACCACGCCTACGTGTTCCTCCTGGTTTTCTCCT
GCCTCGTGCTGTCTGTGTTTTCCACCATCAAGGAGTATGAGAAGAGCTCGGAGGGGG
CCCTCTACATCCTGGAAATCGTGACTATCGTGGTGTTTGGCGTGGAGTACTTCGTGCG
GATCTGGGCCGCAGGCTGCTGCTGCCGGTACCGTGGCTGGAGGGGGCGGCTCAAGTT
TGCCCGGAAACCGTTCTGTGTGATTGACATCATGGTGCTCATCGCCTCCATTGCGGTG
CTGGCCGCCGGCTCCCAGGGCAACGTCTTTGCCACATCTGCGCTCCGGAGCCTGCGC
TTCCTGCAGATTCTGCGGATGATCCGCATGGACCGGGGGGAGGCACCTGGAAGCTG
CTGGGCTCTGTGGTCTATGCCCACAGCAAGGAGCTGGTCACTGCCTGGTACATCGGC
TTCCTTTGTCTCATCCTGGCCTCGTTCCTGGTGTACTTGGCAGAGAAGGGGGAGAAC
GACCACTTTGACACCTACGCGGATGCACTCTGGTGGGGCCTGATCACGCTGACCACC
ATTGGCTACGGGGACAAGTACCCCCAGACCTGGAACGGCAGGCTCCTTGCGGCAAC
CTTCACCCTCATCGGTGTCTCCTTCTTCGCGCTGCCTGCAGGCATCTTGGGGTCTGGG
TTTGCCCTGAAGGTTCAGGAGCAGCACAGGCAGAAGCACTTTGAGAAGAGGCGGAA
CCCGGCAGCAGGCCTGATCCAGTCGGCCTGGAGATTCTACGCCACCAACCTCTCGCG
CACAGACCTGCACTCCACGTGGCAGTACTACGAGCGAACGGTCACCGTGCCCATGTA
CAGTTCGCAAACTCAAACCTACGGGGCCTCCAGACTTATCCCCCCGCTGAACCAGCT
GGAGCTGCTGAGGAACCTCAAGAGTAAATCTGGACTCGCTTTCAGGAAGGACCCCC
CGCCGGAGCCGTCTCCAAGCCAGAAGGTCAGTTTGAAAGATCGTGTCTTCTCCAGCC
CCCGAGGCGTGGCTGCCAAGGGGAAGGGGTCCCCGCAGGCCCAGACTGTGAGGCGG
TCACCCAGCGCCGACCAGAGCCTCGAGGACAGCCCCAGCAAGGTGCCCAAGAGCTG
GAGCTTCGGGGACCGCAGCCGGGCACGCCAGGCTTTCCGCATCAAGGGTGCCGCGT
CACGGCAGAACTCAGAAGAAGCAAGCCTCCCCGGAGAGGACATTGTGGATGACAAG
AGCTGCCCCTGCGAGTTTGTGACCGAGGACCTGACCCCGGGCCTCAAAGTCAGCATC
AGAGCCGTGTGTGTCATGCGGTTCCTGGTGTCCAAGCGGAAGTTCAAGGAGAGCCTG
CGGCCCTACGACGTGATGGACGTCATCGAGCAGTACTCAGCCGGCCACCTGGACAT
GCTGTCCCGAATTAAGAGCCTGCAGTCCAGGATAGATATGATTGTGGGTCCCCCGCC
CCCTTCAACTCCCCGGCACAAGAAGTACCCCACCAAAGGACCCACGGCCCCTCCGA
GAGAGTCACCCCAGTACTCACCTAGAGTGGACCAGATCGTGGGGCGGGGCCCAGCG
ATCACGGACAAGGACCGCACCAAGGGCCCGGCCGAGGCGGAGCTGCCCGAGGACCC
CAGCATGATGGGACGGCTCGGGAAGGTGGAGAAGCAGGTCTTGTCCATGGAGAAGA
AGCTGGACTTCCTGGTGAATATCTACATGCAGCGGATGGGCATCCCCCCGACAGAGA
CCGAGGCCTACTTTGGGGCCAAAGAGCCGGAGCCGGCGCCGCCGTACCACAGCCCG
GAAGACAGCCGGGAGCATGTCGACAGGCACGGCTGCATTGTCAAGATCGTGCGCTC
CAGCAGCTCCACGGGCCAGAAGAACTTCTCGGCGCCCCCGGCCGCGCCCCCTGTCCA
GTGTCCGCCCTCCACCTCCTGGCAGCCACAGAGCCACCCGCGCCAGGGCCACGGCAC
CTCCCCCGTGGGGGACCACGGCTCCCTGGTGCGCATCCCGCCGCCGCCTGCCCACGA
GCGGTCGCTGTCCGCCTACGGCGGGGGCAACCGCGCCAGCATGGAGTTCCTGCGGC
AGGAGGACACCCCGGGCTGCAGGCCCCCCGAGGGGAACCTGCGGGACAGCGACAC
GTCCATCTCCATCCCGTCCGTGGACCACGAGGAGCTGGAGCGTTCCTTCAGCGGCTT
CAGCATCTCCCAGTCCAAGGAGAACCTGGATGCTCTCAACAGCTGCTACGCGGCCGT
GGCGCCTTGTGCCAAAGTCAGGCCCTACATTGCGGAGGGAGAGTCAGACACCGACT
CCGACCTCTGTACCCCGTGCGGGCCCCCGCCACGCTCGGCCACCGGCGAGGGTCCCT
TTGGTGACGTGGGCTGGGCCGGGCCCAGGAAGTGAGGCGGCGCTGGGCCAGTGGAC
CCGCCCGCGGCCCTCCTCAGCACGGTGCCTCCGAGGTTTTGAGGCGGGAACCCTCTG
GGGCCCTTTTCTTACAGTAACTGAGTGTGGCGGGAAGGGTGGGCCCTGGAGGGGCCC
ATGTGGGCTGAAGGATGGGGGCTCCTGGCAGTGACCTTTTACAAAAGTTATTTTCCA
ACAGGGGCTGGAGGGCTGGGCAGGGCCCTGTGGCTCCAGGAGCAGCGTGCAGGAGC
AAGGCTGCCCTGTCCACTCTGCTCAGGGCCGCGGCCGACATCAGCCCGGTGTGAGGA
GGGGGGGGAGTGATGACGGGGTGTTGCCAGCGTGGCAACAGGCGGGGGGTTGTCTC
AGCCGAGCCCAGGGGAGGCACAAAGGGCAGGCCTGTTCCCTGAGGACCTGCGCAAA
GGGCGGGCCTGTTTGGTGAGGACCTGCGGCCTTGGGTCCCGGTGGGGTTTCCGGGCA
GCTACAGGCGGGTGTGGCCGGCCGCTGTGCGTGGCCTCTGCCTTCACACCTGACCTG
CCCGGCGGGCTTTCCTGTTCCCCACCTCAGGGGCGCCCAAATACAGAGCTATTGGTT
GGCGTCTTCTCCCTGTACCTTCTGGGATCTGAGGGCTCTTTCCATGGAAGCCAGCCCC
GAGGTGGAGACCTTCGCCTGCAGCCGAGGAGCGGGTGGGGCCTGGGAACCAAACTG
GAGCCAGAGTGGACGTCCAGCCCTCTGGTCTTGGCCTCCAGAGGGAGGGCCTGGCTC
ACGGTGGGGCCAGGGAGCCGGCTCCAAAGGGTCTTCAAAAAGGGGGTCCTTGGGGG
CTCCAGCTGCCTCGCCCTGGCCTTTCTGTGGGTGCGTGAGAGCCAGCAGCACCCCAG
CCTTGGAGACCGGGGGGGCAGGACCCCAAGTCCTCCCCTCTCTCCTGACTGCCCTGG
CCGGGTGCCGGCACTGCGAGACCCACCTGGTGAGCAGGCCTCACAGTTCTTAGCCAG
GGCCCCACCTCGCCTGTGTCCCACCAGTGCCCCGACAGACCTGGGGCAGGGCTGGGC
CATGATGCAGCGGGCCAGGATAGCCTCCACCGTCAGCACAGGGCCGCCCTCCCCGC
CTTTCCGGAGGAAACCACTCCCACCTCAGCCCAGCTGTGCGCCCTCCCTAGCTCTCCT
GCCCCCTGGAGCTGATGGCCCCTTCTCCACTGACCGATTCCTTAGCGGGGCCTCTTG
GGGTCTCGGGCCTCGGGTGCACCGTCCCATGCCCGTCCTGTTGTGGGCACCGTGGCC
CTTGGGGCAGGCGGCTCTAATGCGGGAGCGAGTCCCTAGCTCCAGACTTAAGAACC
AGACCCCGGGAGCATCTGGCATTTGGCGTGACGGCGTCGCAGGCGGGCCTGGGCTC
CCTGGAGAGTGGCCTCCCTGGGAGTGAGCAGGGCTGGGGTCGTGGGCGCAAATACT
CCTGCAGAGCAAGTGCAGGGGAGTCCTGGGCCCGTTTCTCCTCCACCTGCGTTTTCA
GTGCACTTGGCTTGGCTGGGAGGTCCTGAGGCCCTGAGGCCAGCAGGGGAACCAGT
CCTGAGGGAGAGGACTTTGAAAGCAGCATTTGAGGGTCGTACGCCCCTGGCTGGTG
GGGGTCCTGGCGCTCAGGGTGTTCGGGGAGCCATGTCTGGCGTCCATTGTGGGGAGC
TGCTGCCCTGGCCTCTCTGCCTACCCCCAGCCCGGCCAGGGCACTCCCAGGCCCTGT
CGCCATTGAGGTGCCTCCGCTGGGCTGTCTCCTCACCCCTCCCTGTGCTGGAGCCTGT
CCCAAAAAGGTGCCAACTGGGAGGCCTCGGAAGCCACTGTCCAGGCTCCCACTGCC
TGTCTGCTCTGTTCCCAAAGGCAGCGTGTGTGGCCTCGGGCCCTGCGGTGGCATGAA
GCATCCCTTCTGGTGTGGGCATCGCTACGTGTTTTGGGGGCAGCGTTTCACGGCGGT
GCCCTTGCTGTCTCCCTTGGGCTGGCTCGAGCCTGGGGTCCATGTCCCTTTGCCGTCC
CGTCATGGGGCAGGGAATCCATAGCGGGGCCCACAGGCAGGGGTATGAGTGCGTCC
CACCCAACGCAGCACCAGCCCCGGCCACCGCTCCCCGTGTCCCCAGTTCCGTCTCAG
CTACCTGGACTCCAGGACCCTGGAGAAGGGAGACCTGGCAGTGGAGGGAGGCTGTG
CTGTGTGTCCCCCTGCAGGTGTGACCCCGCCTGCTCTTTCCTCCCCCGCCAGGTGTGG
CCCCGCCTGCTCTTTCCTCCCCCACCAGTATGGCCCCACCTGCTCTTTCCTCCCCCCC
CAAGGTGTGGCCCCACCTGTTCTTTCCTCCCCTGCCGAGGTGTGACCCCACCTGCTCT
TTCCTCCCTCCCAGTATGGCCCCACCTGCTCTTTCCTCCCCCGAGGTGAGGCCCCGCC
TGCTCTTTCCTCCCATGGGGCCGCTGAGGCATGAGCACCTGGGCACAGGTTGGGGCT
CTGCAGGATGAGGAAGACAGGCCAATCCCTTCCCTCCCAGAAGCTGGCCGCCCAGC
AGGAGGGACTGAGGCCAGACTCATGTCCAGCAAGGAACGTGTGGTGTGTCCCCTGG
GAAGTCTCTGGGCCCTGGGAAGAGGGAAGGTGCACGTCCTGGGATGGTTGCGGGGC
CCTGTTTTGGGAGACAAAGGGGTAGAGGGTCTGTCTTGGGCCCCCCCAGACTCTAGC
CTGAGCAGTGCAGCCACCTACTGCCCCACCTCAGAGAAGTGCAGCGGGAAGGAGGC
TGGAGGTGGTGCGGCGCTGCCTCGGGTGTCTGCGTGAATGAGCGTGGCCAAGGACC
AGTGCCACCTCATGGCAAAGAGCTCCCGCAGTGTTTGTTAGAGTGCACATCCCTACG
TGCCCACTGGCACACACACGTGCTCACATACATGTCCGCATACAGGCGTACACATGC
ACGCTTGCACACATGCACACAGACCACATAGCACACATGTGCACTGACCACACCTGT
ATAGACCATGCACAGTACACATACGTGCATACACATGCCTGCATACAGGCATACACA
TGCACGCTTACATGTACACGTGCACAGATCACACACATGCACACACGTGTAGCTCAC
ACACAGTATACACATACACAAGTGCACAGACCACACACAGCACTAACACATGCACA
CACAAAGTGCATAGGCCACACAGCACATGCACACAGGTGCACAGACCACACAGCAC
ACACAAGTGCACAGAGCACACTGCACACATGCACACACACACGCGTGCATGCACAC
TCCTCGCACTTCCAGCCTTGGAGCCCTTCTGTCTCTGGTCTTTCTCTTTGACCCTGCTG
AGTGTAAGCTGCCTGGGGAGGGGCTACAAGGAGTAATTGTGGCTTTAGGGGTCGTG
GTGATGCTGGAATGTCAAGCGCCGTCGTGGGGTATCCGACTGTCCGGGCTCCTGGTC
CGCAGTGGCAGAGCGCCAGGCAGAGCCCAATCAGGGTCTCGTGCTGCCCTTCCCTCC
CACAGCCTGGCAGCCATCCAGAGGAGGGGCTCTACCAGATGCCAAGGTGCCCCGGT
GTCTGTATGGGTGTCCGGTTGGGTCCTGTGTTTGGTCTGCCCTGGAGGTGCTGGGCCC
TCCTGGGATGGGTGGCTCAGCCTCGAATCCCAGGCCCCAGCCCAGGCAGGTGCTGCT
GCCTGTTGTGGTTTCCTGGCCCAGCTTCTCCTTCTCCCTCTGCATAAAATCACAGTCC
GTGAGTCTTCCAGCTGCCACCACGGCTGGGACACGCTGGGGGAGGGCTCCTCCCATG
CCTCCTGCACACAGCCGTCTGAGCAGGGCAGGTGCCCAACACCCCCCACCGGAGGA
CACGCTGCCCCTCAGCGATGCCCCTACCTTTTGGGGGGCCTCGTCTCAAGCCCCCCCT
TGGAGGCTGAAATCACCCCAGGCACTGTGAGGGCTTCTCCAGGGGGCACCCTTTGAG
CTGTGGGTCTGATCACCCCAAGTCCCGCCCGGAGGAGAGGCACAGCCAGGGCGTGT
GGTTTAATGTTTGCCCCCTTCGGGGCTGGAGGTCTCAGTGTTTCTAGATTCCAGACCC
TGCTGCCAGAGAGACCTGCTGCCGGAGAGAAGGGGAGGAGGACTCCAGCTGGGCTC
GGTCCCCCACAGTCAGGGACCCCCATAAAGGACACCCCCTTCTCTCTAGAAAGAGCT
GGGCTCTCAGCTATTTCTAGTTGCTTCCCAGAAGCCGAGGAGCAGAAGGAGCTGTGA
GAGCTTTGCAGAAACGCCCTTGTCCCCGCCCTCCTGAGCTATGAATGCCGTACAGAG
CAGAGGCTGGGGCATTGGCAAGATCACAGGTTGATGCTGCACAGCCCCATTGACAC
AAACCCTCAAAGCAGACGTGAGAGGGACGGTTCACAAAGCTCGGACCTGCCGTGGA
GGGTGCCCGGCAGACGTGGCGTGAGAGGGACGGCTCACGAGGCTTGGACCTGCTGT
GGAGGGTGCCCAGCAGACGTGGTGTGAGAGGAACGGCTCACGAGACTTGGACCTGG
TGGAGGGTGCCCAGCAGACGTGGTGTGAGAGGGACGGCTCACAGGGCTTGGACCTG
CCATGGAGGGTGCCCGGCAGACGTGGTGTGAGAGGGATGGTTCACAGGGCTTGGAC
CTGCCATGGAGGGTGCCCGGCAGATGTGGTGGGAGAGAGATGGCTCATGAGGCTTG
GACCTGCCGTGGAGGGTGCCCAGCAGACGTGGTATGAGAGGGATGGCTCACGAGGC
TTGGACCTGGTGGAGGGTGCCCGGCAGACGTGTGAGAGGGACGGTTCACAAGGCTT
GGACCTGCCATGGAGGGTGCCCAGCAGACGTGGTGTGAGAGGGACAGCTCACGAGG
CTTGGACCTGCTGTGGAGGGTGCCCAGCAGGGGGCTGAGCTCTGAGGGGTGGGTGC
TCAGTGCACGGGTGCCCCCAGTGTCCTCTGATCCTGTCCGGTGCCTCCCCCAACCCCC
ACACCCATGCAGAACTCCCAGGTCACATGCACGTATGTCCAGGGCATGGGGGTGGC
GTGAAGAGGCCTGGTCAGGGCCTTTAGGGGCTGCAGGACGGAATGGCCGCCTGGGG
AGCCTGTGTGGCTGTGCCGGGCAGCCATCCTGCATTCCCACCCAGCGCGCAGTCTCC
ACCTCGGCCCCAGCAAAGCGCTAAGCAGCCGGAGAGACAGCCAGGGCGGCTTCCTG
AAGGATGTGGGATGGTGGACTCCGGGGTCGAGGGAATACGCAGGTTCCTGTCCCTCC
GGGAGACCTAGAGAAGCTGCACACCCAGGAGCTTTCCATGACCCGGGAGCATGAGT
GAATGGGGGTTCCAGTTTGCTGAACTTTGCCGTCTTGTAAGGGTGGGGGCTGACGGC
CGACCCTGGGAGGAGGTGACATCGCCGGGGGAGGTTGTGGGCAACGGTGGAGGAGG
AGAGACGGGAGGGGACCATTTGGGATGGAGGGGCCTCTTCAGAGTTTTAAAAGGCG
TTTGTGGGGTGGAGTTGAGTGTGCTCTGGGCTTGGACACTTGCCGTGGTGCCCCTGG
CTGGCCGAGGAGACTGGCTCTGGCCAGGGGCCCCGTCCTGAGAGGTCCTCAGCGTCT
GACTCTCGGCCAGGCGCCAGCAAGGAGGGGCCGGTCCCCGGGGCTACCAGGCAGGC
ACGTGCACATCGCCATCGCCACACGCCAACTCCGCCTGGGTTTTACAAAGTCGTTGC
CTTAATGCATGTGGACAGGAACTCCCTGAGGTCGCCCCATGCCCCCTGGCTGTGCCA
GGTACGGACGCCCTGGACCCTGCGAACAGGTGGGGGGGGCGAGGGGCCCAAGGGA
CGGGCTCCAGAGACACGCGCAGGGCAGGAGGGGTCTCACGGAGGGGTCTCGCACTG
AGGCGCCCAGAGCTGGTGGTCCCGCTGGACGCCATCCCTCTGCCCGGGATCCACACG
GCCCACGTGTGCCCGCCATGCCCGCGCCCCACGCCATTGCAGTCTGCCATCCTCTGG
CCGTGACGGTGGCTGCAGCTTCCCCATTTGCGCCGTTGCCTCTGGCTGTCTGCACTTT
TGTTCATGCTCCAAAGAACATTTCATAATGCCTTCAGTACCGACGTACACTTCTGACC
ATTTTGTATGTGTCCTTGTGCCGTAGTGACCAGGCCTTTTTTTGGTGGATGTGTTACC
CCGCACACTTCAATCTCAACTTTGTGCACCGTCCATTTTCTAGGGATAGACGCCCAG
GGAATGAACTCTAGTTTTCTAACAGATTAGCTGAGATATTAACTTACTCACACGGAC
AGGTTGATGCCAGAGCCGTAAGAATGCGCCAGTGCGGGTTTGCGGGGGACTTCGGG
TGTGGGGTCCTGCGGCCGCGATGGCCGTGGAAGGTTCTGGGGATCCCTGCTGCCACG
GGGACGAGTTCGGACGCCAGGTGGACCTGTGCACTCAGTAAAACGCAGTGATTCAA
CCTGGA
(SOURCE NCBI REFERENCE NO: NM_001382235.1)

In various embodiments, a KCNQ2 miRNA transcript comprises the sequence provided as SEQ ID NO: 3033.

(SEQ ID NO: 3033)
AGGCGCCGAGGTGCGCGCGGAGCGAGGTGGCCGCAGCGTCTCCGCGCGCGGCCCAA
GCCCGGCAGGAGTGCGGAACCGCCGCCTCGGCCATGCGGCTCCCGGCCGGGGGGCC
TGGGCTGGGGCCCGCGCCGCCCCCCGCGCTCCGCCCCCGCTGAGCCTGAGCCCGACC
CGGGGCGCCTCCCGCCAGGCACCATGGTGCAGAAGTCGCGCAACGGCGGCGTATAC
CCCGGCCCGAGCGGGGAGAAGAAGCTGAAGGTGGGCTTCGTGGGGCTGGACCCCGG
CGCGCCCGACTCCACCCGGGACGGGGCGCTGCTGATCGCCGGCTCCGAGGCCCCCA
AGCGCGGCAGCATCCTCAGCAAACCTCGCGCGGGCGGCGCGGGCGCCGGGAAGCCC
CCCAAGCGCAACGCCTTCTACCGCAAGCTGCAGAATTTCCTCTACAACGTGCTGGAG
CGGCCGCGCGGCTGGGCGTTCATCTACCACGCCTACGTGTTCCTCCTGGTTTTCTCCT
GCCTCGTGCTGTCTGTGTTTTCCACCATCAAGGAGTATGAGAAGAGCTCGGAGGGGG
CCCTCTACATCCTGGAAATCGTGACTATCGTGGTGTTTGGCGTGGAGTACTTCGTGCG
GATCTGGGCCGCAGGCTGCTGCTGCCGGTACCGTGGCTGGAGGGGGCGGCTCAAGTT
TGCCCGGAAACCGTTCTGTGTGATTGACATCATGGTGCTCATCGCCTCCATTGCGGTG
CTGGCCGCCGGCTCCCAGGGCAACGTCTTTGCCACATCTGCGCTCCGGAGCCTGCGC
TTCCTGCAGATTCTGCGGATGATCCGCATGGACCGGCGGGGAGGCACCTGGAAGCTG
CTGGGCTCTGTGGTCTATGCCCACAGCAAGGAGCTGGTCACTGCCTGGTACATCGGC
TTCCTTTGTCTCATCCTGGCCTCGTTCCTGGTGTACTTGGCAGAGAAGGGGGAGAAC
GACCACTTTGACACCTACGCGGATGCACTCTGGTGGGGCCTGATCACGCTGACCACC
ATTGGCTACGGGGACAAGTACCCCCAGACCTGGAACGGCAGGCTCCTTGCGGCAAC
CTTCACCCTCATCGGTGTCTCCTTCTTCGCGCTGCCTGCAGGCATCTTGGGGTCTGGG
TTTGCCCTGAAGGTTCAGGAGCAGCACAGGCAGAAGCACTTTGAGAAGAGGCGGAA
CCCGGCAGCAGGCCTGATCCAGTCGGCCTGGAGATTCTACGCCACCAACCTCTCGCG
CACAGACCTGCACTCCACGTGGCAGTACTACGAGCGAACGGTCACCGTGCCCATGTA
CAGACTTATCCCCCCGCTGAACCAGCTGGAGCTGCTGAGGAACCTCAAGAGTAAATC
TGGACTCGCTTTCAGGAAGGACCCCCCGCCGGAGCCGTCTCCAAGCCAGAAGGTCA
GTTTGAAAGATCGTGTCTTCTCCAGCCCCCGAGGCGTGGCTGCCAAGGGGAAGGGGT
CCCCGCAGGCCCAGACTGTGAGGCGGTCACCCAGCGCCGACCAGAGCCTCGAGGAC
AGCCCCAGCAAGGTGCCCAAGAGCTGGAGCTTCGGGGACCGCAGCCGGGCACGCCA
GGCTTTCCGCATCAAGGGTGCCGCGTCACGGCAGAACTCAGAAGAAGCAAGCCTCC
CCGGAGAGGACATTGTGGATGACAAGAGCTGCCCCTGCGAGTTTGTGACCGAGGAC
CTGACCCCGGGCCTCAAAGTCAGCATCAGAGCCGTGTGTGTCATGCGGTTCCTGGTG
TCCAAGCGGAAGTTCAAGGAGAGCCTGCGGCCCTACGACGTGATGGACGTCATCGA
GCAGTACTCAGCCGGCCACCTGGACATGCTGTCCCGAATTAAGAGCCTGCAGTCCAG
AGTGGACCAGATCGTGGGGCGGGGCCCAGCGATCACGGACAAGGACCGCACCAAG
GGCCCGGCCGAGGCGGAGCTGCCCGAGGACCCCAGCATGATGGGACGGCTCGGGAA
GGTGGAGAAGCAGGTCTTGTCCATGGAGAAGAAGCTGGACTTCCTGGTGAATATCTA
CATGCAGCGGATGGGCATCCCCCCGACAGAGACCGAGGCCTACTTTGGGGCCAAAG
AGCCGGAGCCGGCGCCGCCGTACCACAGCCCGGAAGACAGCCGGGAGCATGTCGAC
AGGCACGGCTGCATTGTCAAGATCGTGCGCTCCAGCAGCTCCACGGGCCAGAAGAA
CTTCTCGGCGCCCCCGGCCGCGCCCCCTGTCCAGTGTCCGCCCTCCACCTCCTGGCAG
CCACAGAGCCACCCGCGCCAGGGCCACGGCACCTCCCCCGTGGGGGACCACGGCTC
CCTGGTGCGCATCCCGCCGCCGCCTGCCCACGAGCGGTCGCTGTCCGCCTACGGCGG
GGGCAACCGCGCCAGCATGGAGTTCCTGCGGCAGGAGGACACCCCGGGCTGCAGGC
CCCCCGAGGGGAACCTGCGGGACAGCGACACGTCCATCTCCATCCCGTCCGTGGACC
ACGAGGAGCTGGAGCGTTCCTTCAGCGGCTTCAGCATCTCCCAGTCCAAGGAGAACC
TGGATGCTCTCAACAGCTGCTACGCGGCCGTGGCGCCTTGTGCCAAAGTCAGGCCCT
ACATTGCGGAGGGAGAGTCAGACACCGACTCCGACCTCTGTACCCCGTGCGGGCCC
CCGCCACGCTCGGCCACCGGCGAGGGTCCCTTTGGTGACGTGGGCTGGGCCGGGCCC
AGGAAGTGAGGCGGCGCTGGGCCAGTGGACCCGCCCGCGGCCCTCCTCAGCACGGT
GCCTCCGAGGTTTTGAGGCGGGAACCCTCTGGGGCCCTTTTCTTACAGTAACTGAGT
GTGGCGGGAAGGGTGGGCCCTGGAGGGGCCCATGTGGGCTGAAGGATGGGGGCTCC
TGGCAGTGACCTTTTACAAAAGTTATTTTCCAACAGGGGCTGGAGGGCTGGGCAGGG
CCCTGTGGCTCCAGGAGCAGCGTGCAGGAGCAAGGCTGCCCTGTCCACTCTGCTCAG
GGCCGCGGCCGACATCAGCCCGGTGTGAGGAGGGGGGGGAGTGATGACGGGGTGTT
GCCAGCGTGGCAACAGGCGGGGGGTTGTCTCAGCCGAGCCCAGGGGAGGCACAAAG
GGCAGGCCTGTTCCCTGAGGACCTGCGCAAAGGGCGGGCCTGTTTGGTGAGGACCTG
CGGCCTTGGGTCCCGGTGGGGTTTCCGGGCAGCTACAGGCGGGTGTGGCCGGCCGCT
GTGCGTGGCCTCTGCCTTCACACCTGACCTGCCCGGCGGGCTTTCCTGTTCCCCACCT
CAGGGGCGCCCAAATACAGAGCTATTGGTTGGCGTCTTCTCCCTGTACCTTCTGGGA
TCTGAGGGCTCTTTCCATGGAAGCCAGCCCCGAGGTGGAGACCTTCGCCTGCAGCCG
AGGAGCGGGTGGGGCCTGGGAACCAAACTGGAGCCAGAGTGGACGTCCAGCCCTCT
GGTCTTGGCCTCCAGAGGGAGGGCCTGGCTCACGGTGGGGCCAGGGAGCCGGCTCC
AAAGGGTCTTCAAAAAGGGGGTCCTTGGGGGCTCCAGCTGCCTCGCCCTGGCCTTTC
TGTGGGTGCGTGAGAGCCAGCAGCACCCCAGCCTTGGAGACCGGGGGGGCAGGACC
CCAAGTCCTCCCCTCTCTCCTGACTGCCCTGGCCGGGTGCCGGCACTGCGAGACCCA
CCTGGTGAGCAGGCCTCACAGTTCTTAGCCAGGGCCCCACCTCGCCTGTGTCCCACC
AGTGCCCCGACAGACCTGGGGCAGGGCTGGGCCATGATGCAGCGGGCCAGGATAGC
CTCCACCGTCAGCACAGGGCCGCCCTCCCCGCCTTTCCGGAGGAAACCACTCCCACC
TCAGCCCAGCTGTGCGCCCTCCCTAGCTCTCCTGCCCCCTGGAGCTGATGGCCCCTTC
TCCACTGACCGATTCCTTAGCGGGGCCTCTTGGGGTCTCGGGCCTCGGGTGCACCGT
CCCATGCCCGTCCTGTTGTGGGCACCGTGGCCCTTGGGGCAGGCGGCTCTAATGCGG
GAGCGAGTCCCTAGCTCCAGACTTAAGAACCAGACCCCGGGAGCATCTGGCATTTGG
CGTGACGGCGTCGCAGGCGGGCCTGGGCTCCCTGGAGAGTGGCCTCCCTGGGAGTG
AGCAGGGCTGGGGTCGTGGGCGCAAATACTCCTGCAGAGCAAGTGCAGGGGAGTCC
TGGGCCCGTTTCTCCTCCACCTGCGTTTTCAGTGCACTTGGCTTGGCTGGGAGGTCCT
GAGGCCCTGAGGCCAGCAGGGGAACCAGTCCTGAGGGAGAGGACTTTGAAAGCAGC
ATTTGAGGGTCGTACGCCCCTGGCTGGTGGGGGTCCTGGCGCTCAGGGTGTTCGGGG
AGCCATGTCTGGCGTCCATTGTGGGGAGCTGCTGCCCTGGCCTCTCTGCCTACCCCC
AGCCCGGCCAGGGCACTCCCAGGCCCTGTCGCCATTGAGGTGCCTCCGCTGGGCTGT
CTCCTCACCCCTCCCTGTGCTGGAGCCTGTCCCAAAAAGGTGCCAACTGGGAGGCCT
CGGAAGCCACTGTCCAGGCTCCCACTGCCTGTCTGCTCTGTTCCCAAAGGCAGCGTG
TGTGGCCTCGGGCCCTGCGGTGGCATGAAGCATCCCTTCTGGTGTGGGCATCGCTAC
GTGTTTTGGGGGCAGCGTTTCACGGCGGTGCCCTTGCTGTCTCCCTTGGGCTGGCTCG
AGCCTGGGGTCCATGTCCCTTTGCCGTCCCGTCATGGGGCAGGGAATCCATAGCGGG
GCCCACAGGCAGGGGTATGAGTGCGTCCCACCCAACGCAGCACCAGCCCCGGCCAC
CGCTCCCCGTGTCCCCAGTTCCGTCTCAGCTACCTGGACTCCAGGACCCTGGAGAAG
GGAGACCTGGCAGTGGAGGGAGGCTGTGCTGTGTGTCCCCCTGCAGGTGTGACCCCG
CCTGCTCTTTCCTCCCCCGCCAGGTGTGGCCCCGCCTGCTCTTTCCTCCCCCACCAGT
ATGGCCCCACCTGCTCTTTCCTCCCCCCCCAAGGTGTGGCCCCACCTGTTCTTTCCTC
CCCTGCCGAGGTGTGACCCCACCTGCTCTTTCCTCCCTCCCAGTATGGCCCCACCTGC
TCTTTCCTCCCCCGAGGTGAGGCCCCGCCTGCTCTTTCCTCCCATGGGGCCGCTGAGG
CATGAGCACCTGGGCACAGGTTGGGGCTCTGCAGGATGAGGAAGACAGGCCAATCC
CTTCCCTCCCAGAAGCTGGCCGCCCAGCAGGAGGGACTGAGGCCAGACTCATGTCC
AGCAAGGAACGTGTGGTGTGTCCCCTGGGAAGTCTCTGGGCCCTGGGAAGAGGGAA
GGTGCACGTCCTGGGATGGTTGCGGGGCCCTGTTTTGGGAGACAAAGGGGTAGAGG
GTCTGTCTTGGGCCCCCCCAGACTCTAGCCTGAGCAGTGCAGCCACCTACTGCCCCA
CCTCAGAGAAGTGCAGCGGGAAGGAGGCTGGAGGTGGTGCGGCGCTGCCTCGGGTG
TCTGCGTGAATGAGCGTGGCCAAGGACCAGTGCCACCTCATGGCAAAGAGCTCCCG
CAGTGTTTGTTAGAGTGCACATCCCTACGTGCCCACTGGCACACACACGTGCTCACA
TACATGTCCGCATACAGGCGTACACATGCACGCTTGCACACATGCACACAGACCACA
TAGCACACATGTGCACTGACCACACCTGTATAGACCATGCACAGTACACATACGTGC
ATACACATGCCTGCATACAGGCATACACATGCACGCTTACATGTACACGTGCACAGA
TCACACACATGCACACACGTGTAGCTCACACACAGTATACACATACACAAGTGCAC
AGACCACACACAGCACTAACACATGCACACACAAAGTGCATAGGCCACACAGCACA
TGCACACAGGTGCACAGACCACACAGCACACACAAGTGCACAGAGCACACTGCACA
CATGCACACACACACGCGTGCATGCACACTCCTCGCACTTCCAGCCTTGGAGCCCTT
CTGTCTCTGGTCTTTCTCTTTGACCCTGCTGAGTGTAAGCTGCCTGGGGAGGGGCTAC
AAGGAGTAATTGTGGCTTTAGGGGTCGTGGTGATGCTGGAATGTCAAGCGCCGTCGT
GGGGTATCCGACTGTCCGGGCTCCTGGTCCGCAGTGGCAGAGCGCCAGGCAGAGCC
CAATCAGGGTCTCGTGCTGCCCTTCCCTCCCACAGCCTGGCAGCCATCCAGAGGAGG
GGCTCTACCAGATGCCAAGGTGCCCCGGTGTCTGTATGGGTGTCCGGTTGGGTCCTG
TGTTTGGTCTGCCCTGGAGGTGCTGGGCCCTCCTGGGATGGGTGGCTCAGCCTCGAA
TCCCAGGCCCCAGCCCAGGCAGGTGCTGCTGCCTGTTGTGGTTTCCTGGCCCAGCTT
CTCCTTCTCCCTCTGCATAAAATCACAGTCCGTGAGTCTTCCAGCTGCCACCACGGCT
GGGACACGCTGGGGGAGGGCTCCTCCCATGCCTCCTGCACACAGCCGTCTGAGCAG
GGCAGGTGCCCAACACCCCCCACCGGAGGACACGCTGCCCCTCAGCGATGCCCCTA
CCTTTTGGGGGGCCTCGTCTCAAGCCCCCCCTTGGAGGCTGAAATCACCCCAGGCAC
TGTGAGGGCTTCTCCAGGGGGCACCCTTTGAGCTGTGGGTCTGATCACCCCAAGTCC
CGCCCGGAGGAGAGGCACAGCCAGGGCGTGTGGTTTAATGTTTGCCCCCTTCGGGGC
TGGAGGTCTCAGTGTTTCTAGATTCCAGACCCTGCTGCCAGAGAGACCTGCTGCCGG
AGAGAAGGGGAGGAGGACTCCAGCTGGGCTCGGTCCCCCACAGTCAGGGACCCCCA
TAAAGGACACCCCCTTCTCTCTAGAAAGAGCTGGGCTCTCAGCTATTTCTAGTTGCTT
CCCAGAAGCCGAGGAGCAGAAGGAGCTGTGAGAGCTTTGCAGAAACGCCCTTGTCC
CCGCCCTCCTGAGCTATGAATGCCGTACAGAGCAGAGGCTGGGGCATTGGCAAGAT
CACAGGTTGATGCTGCACAGCCCCATTGACACAAACCCTCAAAGCAGACGTGAGAG
GGACGGTTCACAAAGCTCGGACCTGCCGTGGAGGGTGCCCGGCAGACGTGGCGTGA
GAGGGACGGCTCACGAGGCTTGGACCTGCTGTGGAGGGTGCCCAGCAGACGTGGTG
TGAGAGGAACGGCTCACGAGACTTGGACCTGGTGGAGGGTGCCCAGCAGACGTGGT
GTGAGAGGGACGGCTCACAGGGCTTGGACCTGCCATGGAGGGTGCCCGGCAGACGT
GGTGTGAGAGGGATGGTTCACAGGGCTTGGACCTGCCATGGAGGGTGCCCGGCAGA
TGTGGTGGGAGAGAGATGGCTCATGAGGCTTGGACCTGCCGTGGAGGGTGCCCAGC
AGACGTGGTATGAGAGGGATGGCTCACGAGGCTTGGACCTGGTGGAGGGTGCCCGG
CAGACGTGTGAGAGGGACGGTTCACAAGGCTTGGACCTGCCATGGAGGGTGCCCAG
CAGACGTGGTGTGAGAGGGACAGCTCACGAGGCTTGGACCTGCTGTGGAGGGTGCC
CAGCAGGGGGCTGAGCTCTGAGGGGTGGGTGCTCAGTGCACGGGTGCCCCCAGTGT
CCTCTGATCCTGTCCGGTGCCTCCCCCAACCCCCACACCCATGCAGAACTCCCAGGT
CACATGCACGTATGTCCAGGGCATGGGGGTGGCGTGAAGAGGCCTGGTCAGGGCCT
TTAGGGGCTGCAGGACGGAATGGCCGCCTGGGGAGCCTGTGTGGCTGTGCCGGGCA
GCCATCCTGCATTCCCACCCAGCGCGCAGTCTCCACCTCGGCCCCAGCAAAGCGCTA
AGCAGCCGGAGAGACAGCCAGGGCGGCTTCCTGAAGGATGTGGGATGGTGGACTCC
GGGGTCGAGGGAATACGCAGGTTCCTGTCCCTCCGGGAGACCTAGAGAAGCTGCAC
ACCCAGGAGCTTTCCATGACCCGGGAGCATGAGTGAATGGGGGTTCCAGTTTGCTGA
ACTTTGCCGTCTTGTAAGGGTGGGGGCTGACGGCCGACCCTGGGAGGAGGTGACATC
GCCGGGGGAGGTTGTGGGCAACGGTGGAGGAGGAGAGACGGGAGGGGACCATTTG
GGATGGAGGGGCCTCTTCAGAGTTTTAAAAGGCGTTTGTGGGGTGGAGTTGAGTGTG
CTCTGGGCTTGGACACTTGCCGTGGTGCCCCTGGCTGGCCGAGGAGACTGGCTCTGG
CCAGGGGCCCCGTCCTGAGAGGTCCTCAGCGTCTGACTCTCGGCCAGGCGCCAGCAA
GGAGGGGCCGGTCCCCGGGGCTACCAGGCAGGCACGTGCACATCGCCATCGCCACA
CGCCAACTCCGCCTGGGTTTTACAAAGTCGTTGCCTTAATGCATGTGGACAGGAACT
CCCTGAGGTCGCCCCATGCCCCCTGGCTGTGCCAGGTACGGACGCCCTGGACCCTGC
GAACAGGTGGGGGGGGCGAGGGGCCCAAGGGACGGGCTCCAGAGACACGCGCAGG
GCAGGAGGGGTCTCACGGAGGGGTCTCGCACTGAGGCGCCCAGAGCTGGTGGTCCC
GCTGGACGCCATCCCTCTGCCCGGGATCCACACGGCCCACGTGTGCCCGCCATGCCC
GCGCCCCACGCCATTGCAGTCTGCCATCCTCTGGCCGTGACGGTGGCTGCAGCTTCC
CCATTTGCGCCGTTGCCTCTGGCTGTCTGCACTTTTGTTCATGCTCCAAAGAACATTT
CATAATGCCTTCAGTACCGACGTACACTTCTGACCATTTTGTATGTGTCCTTGTGCCG
TAGTGACCAGGCCTTTTTTTGGTGGATGTGTTACCCCGCACACTTCAATCTCAACTTT
GTGCACCGTCCATTTTCTAGGGATAGACGCCCAGGGAATGAACTCTAGTTTTCTAAC
AGATTAGCTGAGATATTAACTTACTCACACGGACAGGTTGATGCCAGAGCCGTAAGA
ATGCGCCAGTGCGGGTTTGCGGGGGACTTCGGGTGTGGGGTCCTGCGGCCGCGATGG
CCGTGGAAGGTTCTGGGGATCCCTGCTGCCACGGGGACGAGTTCGGACGCCAGGTG
GACCTGTGCACTCAGTAAAACGCAGTGATTCAACCTGGA
(Source: NCBI Reference Sequence: NM_004518.6).

In various embodiments, a KCNQ2 mRNA transcript comprises the sequence provided as SEQ ID NO: 3034.

(SEQ ID NO: 3034)
AGGCGCCGAGGTGCGCGCGGAGCGAGGTGGCCGCAGCGTCTCCGCGCGCGGCCCAA
GCCCGGCAGGAGTGCGGAACCGCCGCCTCGGCCATGCGGCTCCCGGCCGGGGGGCC
TGGGCTGGGGCCCGCGCCGCCCCCCGCGCTCCGCCCCCGCTGAGCCTGAGCCCGACC
CGGGGCGCCTCCCGCCAGGCACCATGGTGCAGAAGTCGCGCAACGGCGGCGTATAC
CCCGGCCCGAGCGGGGAGAAGAAGCTGAAGGTGGGCTTCGTGGGGCTGGACCCCGG
CGCGCCCGACTCCACCCGGGACGGGGCGCTGCTGATCGCCGGCTCCGAGGCCCCCA
AGCGCGGCAGCATCCTCAGCAAACCTCGCGCGGGCGGCGCGGGCGCCGGGAAGCCC
CCCAAGCGCAACGCCTTCTACCGCAAGCTGCAGAATTTCCTCTACAACGTGCTGGAG
CGGCCGCGCGGCTGGGCGTTCATCTACCACGCCTACGTGTTCCTCCTGGTTTTCTCCT
GCCTCGTGCTGTCTGTGTTTTCCACCATCAAGGAGTATGAGAAGAGCTCGGAGGGGG
CCCTCTACATCCTGGAAATCGTGACTATCGTGGTGTTTGGCGTGGAGTACTTCGTGCG
GATCTGGGCCGCAGGCTGCTGCTGCCGGTACCGTGGCTGGAGGGGGCGGCTCAAGTT
TGCCCGGAAACCGTTCTGTGTGATTGACATCATGGTGCTCATCGCCTCCATTGCGGTG
CTGGCCGCCGGCTCCCAGGGCAACGTCTTTGCCACATCTGCGCTCCGGAGCCTGCGC
TTCCTGCAGATTCTGCGGATGATCCGCATGGACCGGCGGGGAGGCACCTGGAAGCTG
CTGGGCTCTGTGGTCTATGCCCACAGCAAGGAGCTGGTCACTGCCTGGTACATCGGC
TTCCTTTGTCTCATCCTGGCCTCGTTCCTGGTGTACTTGGCAGAGAAGGGGGAGAAC
GACCACTTTGACACCTACGCGGATGCACTCTGGTGGGGCCTGATCACGCTGACCACC
ATTGGCTACGGGGACAAGTACCCCCAGACCTGGAACGGCAGGCTCCTTGCGGCAAC
CTTCACCCTCATCGGTGTCTCCTTCTTCGCGCTGCCTGCAGGCATCTTGGGGTCTGGG
TTTGCCCTGAAGGTTCAGGAGCAGCACAGGCAGAAGCACTTTGAGAAGAGGCGGAA
CCCGGCAGCAGGCCTGATCCAGTCGGCCTGGAGATTCTACGCCACCAACCTCTCGCG
CACAGACCTGCACTCCACGTGGCAGTACTACGAGCGAACGGTCACCGTGCCCATGTA
CAGTTCGCAAACTCAAACCTACGGGGCCTCCAGACTTATCCCCCCGCTGAACCAGCT
GGAGCTGCTGAGGAACCTCAAGAGTAAATCTGGACTCGCTTTCAGGAAGGACCCCC
CGCCGGAGCCGTCTCCAAGCCAGAAGGTCAGTTTGAAAGATCGTGTCTTCTCCAGCC
CCCGAGGCGTGGCTGCCAAGGGGAAGGGGTCCCCGCAGGCCCAGACTGTGAGGCGG
TCACCCAGCGCCGACCAGAGCCTCGAGGACAGCCCCAGCAAGGTGCCCAAGAGCTG
GAGCTTCGGGGACCGCAGCCGGGCACGCCAGGCTTTCCGCATCAAGGGTGCCGCGT
CACGGCAGAACTCAGAAGAAGCAAGCCTCCCCGGAGAGGACATTGTGGATGACAAG
AGCTGCCCCTGCGAGTTTGTGACCGAGGACCTGACCCCGGGCCTCAAAGTCAGCATC
AGAGCCGTGTGTGTCATGCGGTTCCTGGTGTCCAAGCGGAAGTTCAAGGAGAGCCTG
CGGCCCTACGACGTGATGGACGTCATCGAGCAGTACTCAGCCGGCCACCTGGACAT
GCTGTCCCGAATTAAGAGCCTGCAGTCCAGAGTGGACCAGATCGTGGGGCGGGGCC
CAGCGATCACGGACAAGGACCGCACCAAGGGCCCGGCCGAGGCGGAGCTGCCCGA
GGACCCCAGCATGATGGGACGGCTCGGGAAGGTGGAGAAGCAGGTCTTGTCCATGG
AGAAGAAGCTGGACTTCCTGGTGAATATCTACATGCAGCGGATGGGCATCCCCCCGA
CAGAGACCGAGGCCTACTTTGGGGCCAAAGAGCCGGAGCCGGCGCCGCCGTACCAC
AGCCCGGAAGACAGCCGGGAGCATGTCGACAGGCACGGCTGCATTGTCAAGATCGT
GCGCTCCAGCAGCTCCACGGGCCAGAAGAACTTCTCGGCGCCCCCGGCCGCGCCCCC
TGTCCAGTGTCCGCCCTCCACCTCCTGGCAGCCACAGAGCCACCCGCGCCAGGGCCA
CGGCACCTCCCCCGTGGGGGACCACGGCTCCCTGGTGCGCATCCCGCCGCCGCCTGC
CCACGAGCGGTCGCTGTCCGCCTACGGCGGGGGCAACCGCGCCAGCATGGAGTTCC
TGCGGCAGGAGGACACCCCGGGCTGCAGGCCCCCCGAGGGGAACCTGCGGGACAGC
GACACGTCCATCTCCATCCCGTCCGTGGACCACGAGGAGCTGGAGCGTTCCTTCAGC
GGCTTCAGCATCTCCCAGTCCAAGGAGAACCTGGATGCTCTCAACAGCTGCTACGCG
GCCGTGGCGCCTTGTGCCAAAGTCAGGCCCTACATTGCGGAGGGAGAGTCAGACAC
CGACTCCGACCTCTGTACCCCGTGCGGGCCCCCGCCACGCTCGGCCACCGGCGAGGG
TCCCTTTGGTGACGTGGGCTGGGCCGGGCCCAGGAAGTGAGGCGGCGCTGGGCCAG
TGGACCCGCCCGCGGCCCTCCTCAGCACGGTGCCTCCGAGGTTTTGAGGCGGGAACC
CTCTGGGGCCCTTTTCTTACAGTAACTGAGTGTGGCGGGAAGGGTGGGCCCTGGAGG
GGCCCATGTGGGCTGAAGGATGGGGGCTCCTGGCAGTGACCTTTTACAAAAGTTATT
TTCCAACAGGGGCTGGAGGGCTGGGCAGGGCCCTGTGGCTCCAGGAGCAGCGTGCA
GGAGCAAGGCTGCCCTGTCCACTCTGCTCAGGGCCGCGGCCGACATCAGCCCGGTGT
GAGGAGGGGGGGGAGTGATGACGGGGTGTTGCCAGCGTGGCAACAGGCGGGGGGTT
GTCTCAGCCGAGCCCAGGGGAGGCACAAAGGGCAGGCCTGTTCCCTGAGGACCTGC
GCAAAGGGCGGGCCTGTTTGGTGAGGACCTGCGGCCTTGGGTCCCGGTGGGGTTTCC
GGGCAGCTACAGGCGGGTGTGGCCGGCCGCTGTGCGTGGCCTCTGCCTTCACACCTG
ACCTGCCCGGCGGGCTTTCCTGTTCCCCACCTCAGGGGCGCCCAAATACAGAGCTAT
TGGTTGGCGTCTTCTCCCTGTACCTTCTGGGATCTGAGGGCTCTTTCCATGGAAGCCA
GCCCCGAGGTGGAGACCTTCGCCTGCAGCCGAGGAGCGGGTGGGGCCTGGGAACCA
AACTGGAGCCAGAGTGGACGTCCAGCCCTCTGGTCTTGGCCTCCAGAGGGAGGGCCT
GGCTCACGGTGGGGCCAGGGAGCCGGCTCCAAAGGGTCTTCAAAAAGGGGGTCCTT
GGGGGCTCCAGCTGCCTCGCCCTGGCCTTTCTGTGGGTGCGTGAGAGCCAGCAGCAC
CCCAGCCTTGGAGACCGGGGGGGCAGGACCCCAAGTCCTCCCCTCTCTCCTGACTGC
CCTGGCCGGGTGCCGGCACTGCGAGACCCACCTGGTGAGCAGGCCTCACAGTTCTTA
GCCAGGGCCCCACCTCGCCTGTGTCCCACCAGTGCCCCGACAGACCTGGGGCAGGG
CTGGGCCATGATGCAGCGGGCCAGGATAGCCTCCACCGTCAGCACAGGGCCGCCCT
CCCCGCCTTTCCGGAGGAAACCACTCCCACCTCAGCCCAGCTGTGCGCCCTCCCTAG
CTCTCCTGCCCCCTGGAGCTGATGGCCCCTTCTCCACTGACCGATTCCTTAGCGGGGC
CTCTTGGGGTCTCGGGCCTCGGGTGCACCGTCCCATGCCCGTCCTGTTGTGGGCACC
GTGGCCCTTGGGGCAGGCGGCTCTAATGCGGGAGCGAGTCCCTAGCTCCAGACTTAA
GAACCAGACCCCGGGAGCATCTGGCATTTGGCGTGACGGCGTCGCAGGCGGGCCTG
GGCTCCCTGGAGAGTGGCCTCCCTGGGAGTGAGCAGGGCTGGGGTCGTGGGCGCAA
ATACTCCTGCAGAGCAAGTGCAGGGGAGTCCTGGGCCCGTTTCTCCTCCACCTGCGT
TTTCAGTGCACTTGGCTTGGCTGGGAGGTCCTGAGGCCCTGAGGCCAGCAGGGGAAC
CAGTCCTGAGGGAGAGGACTTTGAAAGCAGCATTTGAGGGTCGTACGCCCCTGGCTG
GTGGGGGTCCTGGCGCTCAGGGTGTTCGGGGAGCCATGTCTGGCGTCCATTGTGGGG
AGCTGCTGCCCTGGCCTCTCTGCCTACCCCCAGCCCGGCCAGGGCACTCCCAGGCCC
TGTCGCCATTGAGGTGCCTCCGCTGGGCTGTCTCCTCACCCCTCCCTGTGCTGGAGCC
TGTCCCAAAAAGGTGCCAACTGGGAGGCCTCGGAAGCCACTGTCCAGGCTCCCACT
GCCTGTCTGCTCTGTTCCCAAAGGCAGCGTGTGTGGCCTCGGGCCCTGCGGTGGCAT
GAAGCATCCCTTCTGGTGTGGGCATCGCTACGTGTTTTGGGGGCAGCGTTTCACGGC
GGTGCCCTTGCTGTCTCCCTTGGGCTGGCTCGAGCCTGGGGTCCATGTCCCTTTGCCG
TCCCGTCATGGGGCAGGGAATCCATAGCGGGGCCCACAGGCAGGGGTATGAGTGCG
TCCCACCCAACGCAGCACCAGCCCCGGCCACCGCTCCCCGTGTCCCCAGTTCCGTCT
CAGCTACCTGGACTCCAGGACCCTGGAGAAGGGAGACCTGGCAGTGGAGGGAGGCT
GTGCTGTGTGTCCCCCTGCAGGTGTGACCCCGCCTGCTCTTTCCTCCCCCGCCAGGTG
TGGCCCCGCCTGCTCTTTCCTCCCCCACCAGTATGGCCCCACCTGCTCTTTCCTCCCC
CCCCAAGGTGTGGCCCCACCTGTTCTTTCCTCCCCTGCCGAGGTGTGACCCCACCTGC
TCTTTCCTCCCTCCCAGTATGGCCCCACCTGCTCTTTCCTCCCCCGAGGTGAGGCCCC
GCCTGCTCTTTCCTCCCATGGGGCCGCTGAGGCATGAGCACCTGGGCACAGGTTGGG
GCTCTGCAGGATGAGGAAGACAGGCCAATCCCTTCCCTCCCAGAAGCTGGCCGCCC
AGCAGGAGGGACTGAGGCCAGACTCATGTCCAGCAAGGAACGTGTGGTGTGTCCCC
TGGGAAGTCTCTGGGCCCTGGGAAGAGGGAAGGTGCACGTCCTGGGATGGTTGCGG
GGCCCTGTTTTGGGAGACAAAGGGGTAGAGGGTCTGTCTTGGGCCCCCCCAGACTCT
AGCCTGAGCAGTGCAGCCACCTACTGCCCCACCTCAGAGAAGTGCAGCGGGAAGGA
GGCTGGAGGTGGTGCGGCGCTGCCTCGGGTGTCTGCGTGAATGAGCGTGGCCAAGG
ACCAGTGCCACCTCATGGCAAAGAGCTCCCGCAGTGTTTGTTAGAGTGCACATCCCT
ACGTGCCCACTGGCACACACACGTGCTCACATACATGTCCGCATACAGGCGTACACA
TGCACGCTTGCACACATGCACACAGACCACATAGCACACATGTGCACTGACCACACC
TGTATAGACCATGCACAGTACACATACGTGCATACACATGCCTGCATACAGGCATAC
ACATGCACGCTTACATGTACACGTGCACAGATCACACACATGCACACACGTGTAGCT
CACACACAGTATACACATACACAAGTGCACAGACCACACACAGCACTAACACATGC
ACACACAAAGTGCATAGGCCACACAGCACATGCACACAGGTGCACAGACCACACAG
CACACACAAGTGCACAGAGCACACTGCACACATGCACACACACACGCGTGCATGCA
CACTCCTCGCACTTCCAGCCTTGGAGCCCTTCTGTCTCTGGTCTTTCTCTTTGACCCTG
CTGAGTGTAAGCTGCCTGGGGAGGGGCTACAAGGAGTAATTGTGGCTTTAGGGGTC
GTGGTGATGCTGGAATGTCAAGCGCCGTCGTGGGGTATCCGACTGTCCGGGCTCCTG
GTCCGCAGTGGCAGAGCGCCAGGCAGAGCCCAATCAGGGTCTCGTGCTGCCCTTCCC
TCCCACAGCCTGGCAGCCATCCAGAGGAGGGGCTCTACCAGATGCCAAGGTGCCCC
GGTGTCTGTATGGGTGTCCGGTTGGGTCCTGTGTTTGGTCTGCCCTGGAGGTGCTGGG
CCCTCCTGGGATGGGTGGCTCAGCCTCGAATCCCAGGCCCCAGCCCAGGCAGGTGCT
GCTGCCTGTTGTGGTTTCCTGGCCCAGCTTCTCCTTCTCCCTCTGCATAAAATCACAG
TCCGTGAGTCTTCCAGCTGCCACCACGGCTGGGACACGCTGGGGGAGGGCTCCTCCC
ATGCCTCCTGCACACAGCCGTCTGAGCAGGGCAGGTGCCCAACACCCCCCACCGGA
GGACACGCTGCCCCTCAGCGATGCCCCTACCTTTTGGGGGGCCTCGTCTCAAGCCCC
CCCTTGGAGGCTGAAATCACCCCAGGCACTGTGAGGGCTTCTCCAGGGGGCACCCTT
TGAGCTGTGGGTCTGATCACCCCAAGTCCCGCCCGGAGGAGAGGCACAGCCAGGGC
GTGTGGTTTAATGTTTGCCCCCTTCGGGGCTGGAGGTCTCAGTGTTTCTAGATTCCAG
ACCCTGCTGCCAGAGAGACCTGCTGCCGGAGAGAAGGGGAGGAGGACTCCAGCTGG
GCTCGGTCCCCCACAGTCAGGGACCCCCATAAAGGACACCCCCTTCTCTCTAGAAAG
AGCTGGGCTCTCAGCTATTTCTAGTTGCTTCCCAGAAGCCGAGGAGCAGAAGGAGCT
GTGAGAGCTTTGCAGAAACGCCCTTGTCCCCGCCCTCCTGAGCTATGAATGCCGTAC
AGAGCAGAGGCTGGGGCATTGGCAAGATCACAGGTTGATGCTGCACAGCCCCATTG
ACACAAACCCTCAAAGCAGACGTGAGAGGGACGGTTCACAAAGCTCGGACCTGCCG
TGGAGGGTGCCCGGCAGACGTGGCGTGAGAGGGACGGCTCACGAGGCTTGGACCTG
CTGTGGAGGGTGCCCAGCAGACGTGGTGTGAGAGGAACGGCTCACGAGACTTGGAC
CTGGTGGAGGGTGCCCAGCAGACGTGGTGTGAGAGGGACGGCTCACAGGGCTTGGA
CCTGCCATGGAGGGTGCCCGGCAGACGTGGTGTGAGAGGGATGGTTCACAGGGCTT
GGACCTGCCATGGAGGGTGCCCGGCAGATGTGGTGGGAGAGAGATGGCTCATGAGG
CTTGGACCTGCCGTGGAGGGTGCCCAGCAGACGTGGTATGAGAGGGATGGCTCACG
AGGCTTGGACCTGGTGGAGGGTGCCCGGCAGACGTGTGAGAGGGACGGTTCACAAG
GCTTGGACCTGCCATGGAGGGTGCCCAGCAGACGTGGTGTGAGAGGGACAGCTCAC
GAGGCTTGGACCTGCTGTGGAGGGTGCCCAGCAGGGGGCTGAGCTCTGAGGGGTGG
GTGCTCAGTGCACGGGTGCCCCCAGTGTCCTCTGATCCTGTCCGGTGCCTCCCCCAA
CCCCCACACCCATGCAGAACTCCCAGGTCACATGCACGTATGTCCAGGGCATGGGG
GTGGCGTGAAGAGGCCTGGTCAGGGCCTTTAGGGGCTGCAGGACGGAATGGCCGCC
TGGGGAGCCTGTGTGGCTGTGCCGGGCAGCCATCCTGCATTCCCACCCAGCGCGCAG
TCTCCACCTCGGCCCCAGCAAAGCGCTAAGCAGCCGGAGAGACAGCCAGGGCGGCT
TCCTGAAGGATGTGGGATGGTGGACTCCGGGGTCGAGGGAATACGCAGGTTCCTGTC
CCTCCGGGAGACCTAGAGAAGCTGCACACCCAGGAGCTTTCCATGACCCGGGAGCA
TGAGTGAATGGGGGTTCCAGTTTGCTGAACTTTGCCGTCTTGTAAGGGTGGGGGCTG
ACGGCCGACCCTGGGAGGAGGTGACATCGCCGGGGGAGGTTGTGGGCAACGGTGGA
GGAGGAGAGACGGGAGGGGACCATTTGGGATGGAGGGGCCTCTTCAGAGTTTTAAA
AGGCGTTTGTGGGGTGGAGTTGAGTGTGCTCTGGGCTTGGACACTTGCCGTGGTGCC
CCTGGCTGGCCGAGGAGACTGGCTCTGGCCAGGGGCCCCGTCCTGAGAGGTCCTCA
GCGTCTGACTCTCGGCCAGGCGCCAGCAAGGAGGGGCCGGTCCCCGGGGCTACCAG
GCAGGCACGTGCACATCGCCATCGCCACACGCCAACTCCGCCTGGGTTTTACAAAGT
CGTTGCCTTAATGCATGTGGACAGGAACTCCCTGAGGTCGCCCCATGCCCCCTGGCT
GTGCCAGGTACGGACGCCCTGGACCCTGCGAACAGGTGGGGGGGGCGAGGGGCCCA
AGGGACGGGCTCCAGAGACACGCGCAGGGCAGGAGGGGTCTCACGGAGGGGTCTCG
CACTGAGGCGCCCAGAGCTGGTGGTCCCGCTGGACGCCATCCCTCTGCCCGGGATCC
ACACGGCCCACGTGTGCCCGCCATGCCCGCGCCCCACGCCATTGCAGTCTGCCATCC
TCTGGCCGTGACGGTGGCTGCAGCTTCCCCATTTGCGCCGTTGCCTCTGGCTGTCTGC
ACTTTTGTTCATGCTCCAAAGAACATTTCATAATGCCTTCAGTACCGACGTACACTTC
TGACCATTTTGTATGTGTCCTTGTGCCGTAGTGACCAGGCCTTTTTTTGGTGGATGTG
TTACCCCGCACACTTCAATCTCAACTTTGTGCACCGTCCATTTTCTAGGGATAGACGC
CCAGGGAATGAACTCTAGTTTTCTAACAGATTAGCTGAGATATTAACTTACTCACAC
GGACAGGTTGATGCCAGAGCCGTAAGAATGCGCCAGTGCGGGTTTGCGGGGGACTT
CGGGTGTGGGGTCCTGCGGCCGCGATGGCCGTGGAAGGTTCTGGGGATCCCTGCTGC
CACGGGGACGAGTTCGGACGCCAGGTGGACCTGTGCACTCAGTAAAACGCAGTGAT
TCAACCTGGA
(Source: NCBI Reference Sequence: NM_172106.3).

In various embodiments, a KCNQ2 miRNA transcript comprises the sequence provided as SEQ ID NO: 3035.

(SEQ ID NO: 3035)
AGGCGCCGAGGTGCGCGCGGAGCGAGGTGGCCGCAGCGTCTCCGCGCGCGGCCCAA
GCCCGGCAGGAGTGCGGAACCGCCGCCTCGGCCATGCGGCTCCCGGCCGGGGGGCC
TGGGCTGGGGCCCGCGCCGCCCCCCGCGCTCCGCCCCCGCTGAGCCTGAGCCCGACC
CGGGGCGCCTCCCGCCAGGCACCATGGTGCAGAAGTCGCGCAACGGCGGCGTATAC
CCCGGCCCGAGCGGGGAGAAGAAGCTGAAGGTGGGCTTCGTGGGGCTGGACCCCGG
CGCGCCCGACTCCACCCGGGACGGGGCGCTGCTGATCGCCGGCTCCGAGGCCCCCA
AGCGCGGCAGCATCCTCAGCAAACCTCGCGCGGGCGGCGCGGGCGCCGGGAAGCCC
CCCAAGCGCAACGCCTTCTACCGCAAGCTGCAGAATTTCCTCTACAACGTGCTGGAG
CGGCCGCGCGGCTGGGCGTTCATCTACCACGCCTACGTGTTCCTCCTGGTTTTCTCCT
GCCTCGTGCTGTCTGTGTTTTCCACCATCAAGGAGTATGAGAAGAGCTCGGAGGGGG
CCCTCTACATCCTGGAAATCGTGACTATCGTGGTGTTTGGCGTGGAGTACTTCGTGCG
GATCTGGGCCGCAGGCTGCTGCTGCCGGTACCGTGGCTGGAGGGGGCGGCTCAAGTT
TGCCCGGAAACCGTTCTGTGTGATTGACATCATGGTGCTCATCGCCTCCATTGCGGTG
CTGGCCGCCGGCTCCCAGGGCAACGTCTTTGCCACATCTGCGCTCCGGAGCCTGCGC
TTCCTGCAGATTCTGCGGATGATCCGCATGGACCGGCGGGGAGGCACCTGGAAGCTG
CTGGGCTCTGTGGTCTATGCCCACAGCAAGGAGCTGGTCACTGCCTGGTACATCGGC
TTCCTTTGTCTCATCCTGGCCTCGTTCCTGGTGTACTTGGCAGAGAAGGGGGAGAAC
GACCACTTTGACACCTACGCGGATGCACTCTGGTGGGGCCTGATCACGCTGACCACC
ATTGGCTACGGGGACAAGTACCCCCAGACCTGGAACGGCAGGCTCCTTGCGGCAAC
CTTCACCCTCATCGGTGTCTCCTTCTTCGCGCTGCCTGCAGGCATCTTGGGGTCTGGG
TTTGCCCTGAAGGTTCAGGAGCAGCACAGGCAGAAGCACTTTGAGAAGAGGCGGAA
CCCGGCAGCAGGCCTGATCCAGTCGGCCTGGAGATTCTACGCCACCAACCTCTCGCG
CACAGACCTGCACTCCACGTGGCAGTACTACGAGCGAACGGTCACCGTGCCCATGTA
CAGTTCGCAAACTCAAACCTACGGGGCCTCCAGACTTATCCCCCCGCTGAACCAGCT
GGAGCTGCTGAGGAACCTCAAGAGTAAATCTGGACTCGCTTTCAGGAAGGACCCCC
CGCCGGAGCCGTCTCCAAGTAAAGGCAGCCCGTGCAGAGGGCCCCTGTGTGGATGC
TGCCCCGGACGCTCTAGCCAGAAGGTCAGTTTGAAAGATCGTGTCTTCTCCAGCCCC
CGAGGCGTGGCTGCCAAGGGGAAGGGGTCCCCGCAGGCCCAGACTGTGAGGCGGTC
ACCCAGCGCCGACCAGAGCCTCGAGGACAGCCCCAGCAAGGTGCCCAAGAGCTGGA
GCTTCGGGGACCGCAGCCGGGCACGCCAGGCTTTCCGCATCAAGGGTGCCGCGTCA
CGGCAGAACTCAGAAGAAGCAAGCCTCCCCGGAGAGGACATTGTGGATGACAAGAG
CTGCCCCTGCGAGTTTGTGACCGAGGACCTGACCCCGGGCCTCAAAGTCAGCATCAG
AGCCGTGTGTGTCATGCGGTTCCTGGTGTCCAAGCGGAAGTTCAAGGAGAGCCTGCG
GCCCTACGACGTGATGGACGTCATCGAGCAGTACTCAGCCGGCCACCTGGACATGCT
GTCCCGAATTAAGAGCCTGCAGTCCAGAGTGGACCAGATCGTGGGGCGGGGCCCAG
CGATCACGGACAAGGACCGCACCAAGGGCCCGGCCGAGGCGGAGCTGCCCGAGGA
CCCCAGCATGATGGGACGGCTCGGGAAGGTGGAGAAGCAGGTCTTGTCCATGGAGA
AGAAGCTGGACTTCCTGGTGAATATCTACATGCAGCGGATGGGCATCCCCCCGACAG
AGACCGAGGCCTACTTTGGGGCCAAAGAGCCGGAGCCGGCGCCGCCGTACCACAGC
CCGGAAGACAGCCGGGAGCATGTCGACAGGCACGGCTGCATTGTCAAGATCGTGCG
CTCCAGCAGCTCCACGGGCCAGAAGAACTTCTCGGCGCCCCCGGCCGCGCCCCCTGT
CCAGTGTCCGCCCTCCACCTCCTGGCAGCCACAGAGCCACCCGCGCCAGGGCCACGG
CACCTCCCCCGTGGGGGACCACGGCTCCCTGGTGCGCATCCCGCCGCCGCCTGCCCA
CGAGCGGTCGCTGTCCGCCTACGGCGGGGGCAACCGCGCCAGCATGGAGTTCCTGC
GGCAGGAGGACACCCCGGGCTGCAGGCCCCCCGAGGGGAACCTGCGGGACAGCGA
CACGTCCATCTCCATCCCGTCCGTGGACCACGAGGAGCTGGAGCGTTCCTTCAGCGG
CTTCAGCATCTCCCAGTCCAAGGAGAACCTGGATGCTCTCAACAGCTGCTACGCGGC
CGTGGCGCCTTGTGCCAAAGTCAGGCCCTACATTGCGGAGGGAGAGTCAGACACCG
ACTCCGACCTCTGTACCCCGTGCGGGCCCCCGCCACGCTCGGCCACCGGCGAGGGTC
CCTTTGGTGACGTGGGCTGGGCCGGGCCCAGGAAGTGAGGCGGCGCTGGGCCAGTG
GACCCGCCCGCGGCCCTCCTCAGCACGGTGCCTCCGAGGTTTTGAGGCGGGAACCCT
CTGGGGCCCTTTTCTTACAGTAACTGAGTGTGGCGGGAAGGGTGGGCCCTGGAGGGG
CCCATGTGGGCTGAAGGATGGGGGCTCCTGGCAGTGACCTTTTACAAAAGTTATTTT
CCAACAGGGGCTGGAGGGCTGGGCAGGGCCCTGTGGCTCCAGGAGCAGCGTGCAGG
AGCAAGGCTGCCCTGTCCACTCTGCTCAGGGCCGCGGCCGACATCAGCCCGGTGTGA
GGAGGGGGGGGAGTGATGACGGGGTGTTGCCAGCGTGGCAACAGGCGGGGGGTTGT
CTCAGCCGAGCCCAGGGGAGGCACAAAGGGCAGGCCTGTTCCCTGAGGACCTGCGC
AAAGGGGGGCCTGTTTGGTGAGGACCTGCGGCCTTGGGTCCCGGTGGGGTTTCCGG
GCAGCTACAGGCGGGTGTGGCCGGCCGCTGTGCGTGGCCTCTGCCTTCACACCTGAC
CTGCCCGGCGGGCTTTCCTGTTCCCCACCTCAGGGGCGCCCAAATACAGAGCTATTG
GTTGGCGTCTTCTCCCTGTACCTTCTGGGATCTGAGGGCTCTTTCCATGGAAGCCAGC
CCCGAGGTGGAGACCTTCGCCTGCAGCCGAGGAGCGGGTGGGGCCTGGGAACCAAA
CTGGAGCCAGAGTGGACGTCCAGCCCTCTGGTCTTGGCCTCCAGAGGGAGGGCCTGG
CTCACGGTGGGGCCAGGGAGCCGGCTCCAAAGGGTCTTCAAAAAGGGGGTCCTTGG
GGGCTCCAGCTGCCTCGCCCTGGCCTTTCTGTGGGTGCGTGAGAGCCAGCAGCACCC
CAGCCTTGGAGACCGGGGGGGCAGGACCCCAAGTCCTCCCCTCTCTCCTGACTGCCC
TGGCCGGGTGCCGGCACTGCGAGACCCACCTGGTGAGCAGGCCTCACAGTTCTTAGC
CAGGGCCCCACCTCGCCTGTGTCCCACCAGTGCCCCGACAGACCTGGGGCAGGGCTG
GGCCATGATGCAGCGGGCCAGGATAGCCTCCACCGTCAGCACAGGGCCGCCCTCCC
CGCCTTTCCGGAGGAAACCACTCCCACCTCAGCCCAGCTGTGCGCCCTCCCTAGCTC
TCCTGCCCCCTGGAGCTGATGGCCCCTTCTCCACTGACCGATTCCTTAGCGGGGCCTC
TTGGGGTCTCGGGCCTCGGGTGCACCGTCCCATGCCCGTCCTGTTGTGGGCACCGTG
GCCCTTGGGGCAGGCGGCTCTAATGCGGGAGCGAGTCCCTAGCTCCAGACTTAAGA
ACCAGACCCCGGGAGCATCTGGCATTTGGCGTGACGGCGTCGCAGGCGGGCCTGGG
CTCCCTGGAGAGTGGCCTCCCTGGGAGTGAGCAGGGCTGGGGTCGTGGGCGCAAAT
ACTCCTGCAGAGCAAGTGCAGGGGAGTCCTGGGCCCGTTTCTCCTCCACCTGCGTTT
TCAGTGCACTTGGCTTGGCTGGGAGGTCCTGAGGCCCTGAGGCCAGCAGGGGAACC
AGTCCTGAGGGAGAGGACTTTGAAAGCAGCATTTGAGGGTCGTACGCCCCTGGCTG
GTGGGGGTCCTGGCGCTCAGGGTGTTCGGGGAGCCATGTCTGGCGTCCATTGTGGGG
AGCTGCTGCCCTGGCCTCTCTGCCTACCCCCAGCCCGGCCAGGGCACTCCCAGGCCC
TGTCGCCATTGAGGTGCCTCCGCTGGGCTGTCTCCTCACCCCTCCCTGTGCTGGAGCC
TGTCCCAAAAAGGTGCCAACTGGGAGGCCTCGGAAGCCACTGTCCAGGCTCCCACT
GCCTGTCTGCTCTGTTCCCAAAGGCAGCGTGTGTGGCCTCGGGCCCTGCGGTGGCAT
GAAGCATCCCTTCTGGTGTGGGCATCGCTACGTGTTTTGGGGGCAGCGTTTCACGGC
GGTGCCCTTGCTGTCTCCCTTGGGCTGGCTCGAGCCTGGGGTCCATGTCCCTTTGCCG
TCCCGTCATGGGGCAGGGAATCCATAGCGGGGCCCACAGGCAGGGGTATGAGTGCG
TCCCACCCAACGCAGCACCAGCCCCGGCCACCGCTCCCCGTGTCCCCAGTTCCGTCT
CAGCTACCTGGACTCCAGGACCCTGGAGAAGGGAGACCTGGCAGTGGAGGGAGGCT
GTGCTGTGTGTCCCCCTGCAGGTGTGACCCCGCCTGCTCTTTCCTCCCCCGCCAGGTG
TGGCCCCGCCTGCTCTTTCCTCCCCCACCAGTATGGCCCCACCTGCTCTTTCCTCCCC
CCCCAAGGTGTGGCCCCACCTGTTCTTTCCTCCCCTGCCGAGGTGTGACCCCACCTGC
TCTTTCCTCCCTCCCAGTATGGCCCCACCTGCTCTTTCCTCCCCCGAGGTGAGGCCCC
GCCTGCTCTTTCCTCCCATGGGGCCGCTGAGGCATGAGCACCTGGGCACAGGTTGGG
GCTCTGCAGGATGAGGAAGACAGGCCAATCCCTTCCCTCCCAGAAGCTGGCCGCCC
AGCAGGAGGGACTGAGGCCAGACTCATGTCCAGCAAGGAACGTGTGGTGTGTCCCC
TGGGAAGTCTCTGGGCCCTGGGAAGAGGGAAGGTGCACGTCCTGGGATGGTTGCGG
GGCCCTGTTTTGGGAGACAAAGGGGTAGAGGGTCTGTCTTGGGCCCCCCCAGACTCT
AGCCTGAGCAGTGCAGCCACCTACTGCCCCACCTCAGAGAAGTGCAGCGGGAAGGA
GGCTGGAGGTGGTGCGGCGCTGCCTCGGGTGTCTGCGTGAATGAGCGTGGCCAAGG
ACCAGTGCCACCTCATGGCAAAGAGCTCCCGCAGTGTTTGTTAGAGTGCACATCCCT
ACGTGCCCACTGGCACACACACGTGCTCACATACATGTCCGCATACAGGCGTACACA
TGCACGCTTGCACACATGCACACAGACCACATAGCACACATGTGCACTGACCACACC
TGTATAGACCATGCACAGTACACATACGTGCATACACATGCCTGCATACAGGCATAC
ACATGCACGCTTACATGTACACGTGCACAGATCACACACATGCACACACGTGTAGCT
CACACACAGTATACACATACACAAGTGCACAGACCACACACAGCACTAACACATGC
ACACACAAAGTGCATAGGCCACACAGCACATGCACACAGGTGCACAGACCACACAG
CACACACAAGTGCACAGAGCACACTGCACACATGCACACACACACGCGTGCATGCA
CACTCCTCGCACTTCCAGCCTTGGAGCCCTTCTGTCTCTGGTCTTTCTCTTTGACCCTG
CTGAGTGTAAGCTGCCTGGGGAGGGGCTACAAGGAGTAATTGTGGCTTTAGGGGTC
GTGGTGATGCTGGAATGTCAAGCGCCGTCGTGGGGTATCCGACTGTCCGGGCTCCTG
GTCCGCAGTGGCAGAGCGCCAGGCAGAGCCCAATCAGGGTCTCGTGCTGCCCTTCCC
TCCCACAGCCTGGCAGCCATCCAGAGGAGGGGCTCTACCAGATGCCAAGGTGCCCC
GGTGTCTGTATGGGTGTCCGGTTGGGTCCTGTGTTTGGTCTGCCCTGGAGGTGCTGGG
CCCTCCTGGGATGGGTGGCTCAGCCTCGAATCCCAGGCCCCAGCCCAGGCAGGTGCT
GCTGCCTGTTGTGGTTTCCTGGCCCAGCTTCTCCTTCTCCCTCTGCATAAAATCACAG
TCCGTGAGTCTTCCAGCTGCCACCACGGCTGGGACACGCTGGGGGAGGGCTCCTCCC
ATGCCTCCTGCACACAGCCGTCTGAGCAGGGCAGGTGCCCAACACCCCCCACCGGA
GGACACGCTGCCCCTCAGCGATGCCCCTACCTTTTGGGGGGCCTCGTCTCAAGCCCC
CCCTTGGAGGCTGAAATCACCCCAGGCACTGTGAGGGCTTCTCCAGGGGGCACCCTT
TGAGCTGTGGGTCTGATCACCCCAAGTCCCGCCCGGAGGAGAGGCACAGCCAGGGC
GTGTGGTTTAATGTTTGCCCCCTTCGGGGCTGGAGGTCTCAGTGTTTCTAGATTCCAG
ACCCTGCTGCCAGAGAGACCTGCTGCCGGAGAGAAGGGGAGGAGGACTCCAGCTGG
GCTCGGTCCCCCACAGTCAGGGACCCCCATAAAGGACACCCCCTTCTCTCTAGAAAG
AGCTGGGCTCTCAGCTATTTCTAGTTGCTTCCCAGAAGCCGAGGAGCAGAAGGAGCT
GTGAGAGCTTTGCAGAAACGCCCTTGTCCCCGCCCTCCTGAGCTATGAATGCCGTAC
AGAGCAGAGGCTGGGGCATTGGCAAGATCACAGGTTGATGCTGCACAGCCCCATTG
ACACAAACCCTCAAAGCAGACGTGAGAGGGACGGTTCACAAAGCTCGGACCTGCCG
TGGAGGGTGCCCGGCAGACGTGGCGTGAGAGGGACGGCTCACGAGGCTTGGACCTG
CTGTGGAGGGTGCCCAGCAGACGTGGTGTGAGAGGAACGGCTCACGAGACTTGGAC
CTGGTGGAGGGTGCCCAGCAGACGTGGTGTGAGAGGGACGGCTCACAGGGCTTGGA
CCTGCCATGGAGGGTGCCCGGCAGACGTGGTGTGAGAGGGATGGTTCACAGGGCTT
GGACCTGCCATGGAGGGTGCCCGGCAGATGTGGTGGGAGAGAGATGGCTCATGAGG
CTTGGACCTGCCGTGGAGGGTGCCCAGCAGACGTGGTATGAGAGGGATGGCTCACG
AGGCTTGGACCTGGTGGAGGGTGCCCGGCAGACGTGTGAGAGGGACGGTTCACAAG
GCTTGGACCTGCCATGGAGGGTGCCCAGCAGACGTGGTGTGAGAGGGACAGCTCAC
GAGGCTTGGACCTGCTGTGGAGGGTGCCCAGCAGGGGGCTGAGCTCTGAGGGGTGG
GTGCTCAGTGCACGGGTGCCCCCAGTGTCCTCTGATCCTGTCCGGTGCCTCCCCCAA
CCCCCACACCCATGCAGAACTCCCAGGTCACATGCACGTATGTCCAGGGCATGGGG
GTGGCGTGAAGAGGCCTGGTCAGGGCCTTTAGGGGCTGCAGGACGGAATGGCCGCC
TGGGGAGCCTGTGTGGCTGTGCCGGGCAGCCATCCTGCATTCCCACCCAGCGCGCAG
TCTCCACCTCGGCCCCAGCAAAGCGCTAAGCAGCCGGAGAGACAGCCAGGGCGGCT
TCCTGAAGGATGTGGGATGGTGGACTCCGGGGTCGAGGGAATACGCAGGTTCCTGTC
CCTCCGGGAGACCTAGAGAAGCTGCACACCCAGGAGCTTTCCATGACCCGGGAGCA
TGAGTGAATGGGGGTTCCAGTTTGCTGAACTTTGCCGTCTTGTAAGGGTGGGGGCTG
ACGGCCGACCCTGGGAGGAGGTGACATCGCCGGGGGAGGTTGTGGGCAACGGTGGA
GGAGGAGAGACGGGAGGGGACCATTTGGGATGGAGGGGCCTCTTCAGAGTTTTAAA
AGGCGTTTGTGGGGTGGAGTTGAGTGTGCTCTGGGCTTGGACACTTGCCGTGGTGCC
CCTGGCTGGCCGAGGAGACTGGCTCTGGCCAGGGGCCCCGTCCTGAGAGGTCCTCA
GCGTCTGACTCTCGGCCAGGCGCCAGCAAGGAGGGGCCGGTCCCCGGGGCTACCAG
GCAGGCACGTGCACATCGCCATCGCCACACGCCAACTCCGCCTGGGTTTTACAAAGT
CGTTGCCTTAATGCATGTGGACAGGAACTCCCTGAGGTCGCCCCATGCCCCCTGGCT
GTGCCAGGTACGGACGCCCTGGACCCTGCGAACAGGTGGGGGGGGCGAGGGGCCCA
AGGGACGGGCTCCAGAGACACGCGCAGGGCAGGAGGGGTCTCACGGAGGGGTCTCG
CACTGAGGCGCCCAGAGCTGGTGGTCCCGCTGGACGCCATCCCTCTGCCCGGGATCC
ACACGGCCCACGTGTGCCCGCCATGCCCGCGCCCCACGCCATTGCAGTCTGCCATCC
TCTGGCCGTGACGGTGGCTGCAGCTTCCCCATTTGCGCCGTTGCCTCTGGCTGTCTGC
ACTTTTGTTCATGCTCCAAAGAACATTTCATAATGCCTTCAGTACCGACGTACACTTC
TGACCATTTTGTATGTGTCCTTGTGCCGTAGTGACCAGGCCTTTTTTTGGTGGATGTG
TTACCCCGCACACTTCAATCTCAACTTTGTGCACCGTCCATTTTCTAGGGATAGACGC
CCAGGGAATGAACTCTAGTTTTCTAACAGATTAGCTGAGATATTAACTTACTCACAC
GGACAGGTTGATGCCAGAGCCGTAAGAATGCGCCAGTGCGGGTTTGCGGGGGACTT
CGGGTGTGGGGTCCTGCGGCCGCGATGGCCGTGGAAGGTTCTGGGGATCCCTGCTGC
CACGGGGACGAGTTCGGACGCCAGGTGGACCTGTGCACTCAGTAAAACGCAGTGAT
TCAACCTGGA
(Source: NCBI Reference Sequence: NM_172107.4 ).

In various embodiments, a KCNQ2 miRNA transcript comprises the sequence provided as SEQ ID NO: 3036.

(SEQ ID NO: 3036)
AGGCGCCGAGGTGCGCGCGGAGCGAGGTGGCCGCAGCGTCTCCGCGCGCGGCCCAA
GCCCGGCAGGAGTGCGGAACCGCCGCCTCGGCCATGCGGCTCCCGGCCGGGGGGCC
TGGGCTGGGGCCCGCGCCGCCCCCCGCGCTCCGCCCCCGCTGAGCCTGAGCCCGACC
CGGGGCGCCTCCCGCCAGGCACCATGGTGCAGAAGTCGCGCAACGGCGGCGTATAC
CCCGGCCCGAGCGGGGAGAAGAAGCTGAAGGTGGGCTTCGTGGGGCTGGACCCCGG
CGCGCCCGACTCCACCCGGGACGGGGCGCTGCTGATCGCCGGCTCCGAGGCCCCCA
AGCGCGGCAGCATCCTCAGCAAACCTCGCGCGGGCGGCGCGGGCGCCGGGAAGCCC
CCCAAGCGCAACGCCTTCTACCGCAAGCTGCAGAATTTCCTCTACAACGTGCTGGAG
CGGCCGCGCGGCTGGGCGTTCATCTACCACGCCTACGTGTTCCTCCTGGTTTTCTCCT
GCCTCGTGCTGTCTGTGTTTTCCACCATCAAGGAGTATGAGAAGAGCTCGGAGGGGG
CCCTCTACATCCTGGAAATCGTGACTATCGTGGTGTTTGGCGTGGAGTACTTCGTGCG
GATCTGGGCCGCAGGCTGCTGCTGCCGGTACCGTGGCTGGAGGGGGCGGCTCAAGTT
TGCCCGGAAACCGTTCTGTGTGATTGACATCATGGTGCTCATCGCCTCCATTGCGGTG
CTGGCCGCCGGCTCCCAGGGCAACGTCTTTGCCACATCTGCGCTCCGGAGCCTGCGC
TTCCTGCAGATTCTGCGGATGATCCGCATGGACCGGCGGGGAGGCACCTGGAAGCTG
CTGGGCTCTGTGGTCTATGCCCACAGCAAGGAGCTGGTCACTGCCTGGTACATCGGC
TTCCTTTGTCTCATCCTGGCCTCGTTCCTGGTGTACTTGGCAGAGAAGGGGGAGAAC
GACCACTTTGACACCTACGCGGATGCACTCTGGTGGGGCCTGATCACGCTGACCACC
ATTGGCTACGGGGACAAGTACCCCCAGACCTGGAACGGCAGGCTCCTTGCGGCAAC
CTTCACCCTCATCGGTGTCTCCTTCTTCGCGCTGCCTGCAGGCATCTTGGGGTCTGGG
TTTGCCCTGAAGGTTCAGGAGCAGCACAGGCAGAAGCACTTTGAGAAGAGGCGGAA
CCCGGCAGCAGGCCTGATCCAGTCGGCCTGGAGATTCTACGCCACCAACCTCTCGCG
CACAGACCTGCACTCCACGTGGCAGTACTACGAGCGAACGGTCACCGTGCCCATGTA
CAGTTCGCAAACTCAAACCTACGGGGCCTCCAGACTTATCCCCCCGCTGAACCAGCT
GGAGCTGCTGAGGAACCTCAAGAGTAAATCTGGACTCGCTTTCAGGAAGGACCCCC
CGCCGGAGCCGTCTCCAAGCCCCCGAGGCGTGGCTGCCAAGGGGAAGGGGTCCCCG
CAGGCCCAGACTGTGAGGCGGTCACCCAGCGCCGACCAGAGCCTCGAGGACAGCCC
CAGCAAGGTGCCCAAGAGCTGGAGCTTCGGGGACCGCAGCCGGGCACGCCAGGCTT
TCCGCATCAAGGGTGCCGCGTCACGGCAGAACTCAGAAGCAAGCCTCCCCGGAGAG
GACATTGTGGATGACAAGAGCTGCCCCTGCGAGTTTGTGACCGAGGACCTGACCCCG
GGCCTCAAAGTCAGCATCAGAGCCGTGTGTGTCATGCGGTTCCTGGTGTCCAAGCGG
AAGTTCAAGGAGAGCCTGCGGCCCTACGACGTGATGGACGTCATCGAGCAGTACTC
AGCCGGCCACCTGGACATGCTGTCCCGAATTAAGAGCCTGCAGTCCAGAGTGGACC
AGATCGTGGGGGGGGCCCAGCGATCACGGACAAGGACCGCACCAAGGGCCCGGCC
GAGGCGGAGCTGCCCGAGGACCCCAGCATGATGGGACGGCTCGGGAAGGTGGAGA
AGCAGGTCTTGTCCATGGAGAAGAAGCTGGACTTCCTGGTGAATATCTACATGCAGC
GGATGGGCATCCCCCCGACAGAGACCGAGGCCTACTTTGGGGCCAAAGAGCCGGAG
CCGGCGCCGCCGTACCACAGCCCGGAAGACAGCCGGGAGCATGTCGACAGGCACGG
CTGCATTGTCAAGATCGTGCGCTCCAGCAGCTCCACGGGCCAGAAGAACTTCTCGGC
GCCCCCGGCCGCGCCCCCTGTCCAGTGTCCGCCCTCCACCTCCTGGCAGCCACAGAG
CCACCCGCGCCAGGGCCACGGCACCTCCCCCGTGGGGGACCACGGCTCCCTGGTGC
GCATCCCGCCGCCGCCTGCCCACGAGCGGTCGCTGTCCGCCTACGGCGGGGGCAACC
GCGCCAGCATGGAGTTCCTGCGGCAGGAGGACACCCCGGGCTGCAGGCCCCCCGAG
GGGAACCTGCGGGACAGCGACACGTCCATCTCCATCCCGTCCGTGGACCACGAGGA
GCTGGAGCGTTCCTTCAGCGGCTTCAGCATCTCCCAGTCCAAGGAGAACCTGGATGC
TCTCAACAGCTGCTACGCGGCCGTGGCGCCTTGTGCCAAAGTCAGGCCCTACATTGC
GGAGGGAGAGTCAGACACCGACTCCGACCTCTGTACCCCGTGCGGGCCCCCGCCAC
GCTCGGCCACCGGCGAGGGTCCCTTTGGTGACGTGGGCTGGGCCGGGCCCAGGAAG
TGAGGCGGCGCTGGGCCAGTGGACCCGCCCGCGGCCCTCCTCAGCACGGTGCCTCCG
AGGTTTTGAGGCGGGAACCCTCTGGGGCCCTTTTCTTACAGTAACTGAGTGTGGCGG
GAAGGGTGGGCCCTGGAGGGGCCCATGTGGGCTGAAGGATGGGGGCTCCTGGCAGT
GACCTTTTACAAAAGTTATTTTCCAACAGGGGCTGGAGGGCTGGGCAGGGCCCTGTG
GCTCCAGGAGCAGCGTGCAGGAGCAAGGCTGCCCTGTCCACTCTGCTCAGGGCCGC
GGCCGACATCAGCCCGGTGTGAGGAGGGGGGGGAGTGATGACGGGGTGTTGCCAGC
GTGGCAACAGGCGGGGGGTTGTCTCAGCCGAGCCCAGGGGAGGCACAAAGGGCAG
GCCTGTTCCCTGAGGACCTGCGCAAAGGGCGGGCCTGTTTGGTGAGGACCTGCGGCC
TTGGGTCCCGGTGGGGTTTCCGGGCAGCTACAGGCGGGTGTGGCCGGCCGCTGTGCG
TGGCCTCTGCCTTCACACCTGACCTGCCCGGCGGGCTTTCCTGTTCCCCACCTCAGGG
GCGCCCAAATACAGAGCTATTGGTTGGCGTCTTCTCCCTGTACCTTCTGGGATCTGAG
GGCTCTTTCCATGGAAGCCAGCCCCGAGGTGGAGACCTTCGCCTGCAGCCGAGGAG
CGGGTGGGGCCTGGGAACCAAACTGGAGCCAGAGTGGACGTCCAGCCCTCTGGTCT
TGGCCTCCAGAGGGAGGGCCTGGCTCACGGTGGGGCCAGGGAGCCGGCTCCAAAGG
GTCTTCAAAAAGGGGGTCCTTGGGGGCTCCAGCTGCCTCGCCCTGGCCTTTCTGTGG
GTGCGTGAGAGCCAGCAGCACCCCAGCCTTGGAGACCGGGGGGGCAGGACCCCAAG
TCCTCCCCTCTCTCCTGACTGCCCTGGCCGGGTGCCGGCACTGCGAGACCCACCTGG
TGAGCAGGCCTCACAGTTCTTAGCCAGGGCCCCACCTCGCCTGTGTCCCACCAGTGC
CCCGACAGACCTGGGGCAGGGCTGGGCCATGATGCAGCGGGCCAGGATAGCCTCCA
CCGTCAGCACAGGGCCGCCCTCCCCGCCTTTCCGGAGGAAACCACTCCCACCTCAGC
CCAGCTGTGCGCCCTCCCTAGCTCTCCTGCCCCCTGGAGCTGATGGCCCCTTCTCCAC
TGACCGATTCCTTAGCGGGGCCTCTTGGGGTCTCGGGCCTCGGGTGCACCGTCCCAT
GCCCGTCCTGTTGTGGGCACCGTGGCCCTTGGGGCAGGCGGCTCTAATGCGGGAGCG
AGTCCCTAGCTCCAGACTTAAGAACCAGACCCCGGGAGCATCTGGCATTTGGCGTGA
CGGCGTCGCAGGCGGGCCTGGGCTCCCTGGAGAGTGGCCTCCCTGGGAGTGAGCAG
GGCTGGGGTCGTGGGCGCAAATACTCCTGCAGAGCAAGTGCAGGGGAGTCCTGGGC
CCGTTTCTCCTCCACCTGCGTTTTCAGTGCACTTGGCTTGGCTGGGAGGTCCTGAGGC
CCTGAGGCCAGCAGGGGAACCAGTCCTGAGGGAGAGGACTTTGAAAGCAGCATTTG
AGGGTCGTACGCCCCTGGCTGGTGGGGGTCCTGGCGCTCAGGGTGTTCGGGGAGCCA
TGTCTGGCGTCCATTGTGGGGAGCTGCTGCCCTGGCCTCTCTGCCTACCCCCAGCCCG
GCCAGGGCACTCCCAGGCCCTGTCGCCATTGAGGTGCCTCCGCTGGGCTGTCTCCTC
ACCCCTCCCTGTGCTGGAGCCTGTCCCAAAAAGGTGCCAACTGGGAGGCCTCGGAA
GCCACTGTCCAGGCTCCCACTGCCTGTCTGCTCTGTTCCCAAAGGCAGCGTGTGTGG
CCTCGGGCCCTGCGGTGGCATGAAGCATCCCTTCTGGTGTGGGCATCGCTACGTGTT
TTGGGGGCAGCGTTTCACGGCGGTGCCCTTGCTGTCTCCCTTGGGCTGGCTCGAGCCT
GGGGTCCATGTCCCTTTGCCGTCCCGTCATGGGGCAGGGAATCCATAGCGGGGCCCA
CAGGCAGGGGTATGAGTGCGTCCCACCCAACGCAGCACCAGCCCCGGCCACCGCTC
CCCGTGTCCCCAGTTCCGTCTCAGCTACCTGGACTCCAGGACCCTGGAGAAGGGAGA
CCTGGCAGTGGAGGGAGGCTGTGCTGTGTGTCCCCCTGCAGGTGTGACCCCGCCTGC
TCTTTCCTCCCCCGCCAGGTGTGGCCCCGCCTGCTCTTTCCTCCCCCACCAGTATGGC
CCCACCTGCTCTTTCCTCCCCCCCCAAGGTGTGGCCCCACCTGTTCTTTCCTCCCCTG
CCGAGGTGTGACCCCACCTGCTCTTTCCTCCCTCCCAGTATGGCCCCACCTGCTCTTT
CCTCCCCCGAGGTGAGGCCCCGCCTGCTCTTTCCTCCCATGGGGCCGCTGAGGCATG
AGCACCTGGGCACAGGTTGGGGCTCTGCAGGATGAGGAAGACAGGCCAATCCCTTC
CCTCCCAGAAGCTGGCCGCCCAGCAGGAGGGACTGAGGCCAGACTCATGTCCAGCA
AGGAACGTGTGGTGTGTCCCCTGGGAAGTCTCTGGGCCCTGGGAAGAGGGAAGGTG
CACGTCCTGGGATGGTTGCGGGGCCCTGTTTTGGGAGACAAAGGGGTAGAGGGTCT
GTCTTGGGCCCCCCCAGACTCTAGCCTGAGCAGTGCAGCCACCTACTGCCCCACCTC
AGAGAAGTGCAGCGGGAAGGAGGCTGGAGGTGGTGCGGCGCTGCCTCGGGTGTCTG
CGTGAATGAGCGTGGCCAAGGACCAGTGCCACCTCATGGCAAAGAGCTCCCGCAGT
GTTTGTTAGAGTGCACATCCCTACGTGCCCACTGGCACACACACGTGCTCACATACA
TGTCCGCATACAGGCGTACACATGCACGCTTGCACACATGCACACAGACCACATAGC
ACACATGTGCACTGACCACACCTGTATAGACCATGCACAGTACACATACGTGCATAC
ACATGCCTGCATACAGGCATACACATGCACGCTTACATGTACACGTGCACAGATCAC
ACACATGCACACACGTGTAGCTCACACACAGTATACACATACACAAGTGCACAGAC
CACACACAGCACTAACACATGCACACACAAAGTGCATAGGCCACACAGCACATGCA
CACAGGTGCACAGACCACACAGCACACACAAGTGCACAGAGCACACTGCACACATG
CACACACACACGCGTGCATGCACACTCCTCGCACTTCCAGCCTTGGAGCCCTTCTGT
CTCTGGTCTTTCTCTTTGACCCTGCTGAGTGTAAGCTGCCTGGGGAGGGGCTACAAG
GAGTAATTGTGGCTTTAGGGGTCGTGGTGATGCTGGAATGTCAAGCGCCGTCGTGGG
GTATCCGACTGTCCGGGCTCCTGGTCCGCAGTGGCAGAGCGCCAGGCAGAGCCCAA
TCAGGGTCTCGTGCTGCCCTTCCCTCCCACAGCCTGGCAGCCATCCAGAGGAGGGGC
TCTACCAGATGCCAAGGTGCCCCGGTGTCTGTATGGGTGTCCGGTTGGGTCCTGTGTT
TGGTCTGCCCTGGAGGTGCTGGGCCCTCCTGGGATGGGTGGCTCAGCCTCGAATCCC
AGGCCCCAGCCCAGGCAGGTGCTGCTGCCTGTTGTGGTTTCCTGGCCCAGCTTCTCCT
TCTCCCTCTGCATAAAATCACAGTCCGTGAGTCTTCCAGCTGCCACCACGGCTGGGA
CACGCTGGGGGAGGGCTCCTCCCATGCCTCCTGCACACAGCCGTCTGAGCAGGGCA
GGTGCCCAACACCCCCCACCGGAGGACACGCTGCCCCTCAGCGATGCCCCTACCTTT
TGGGGGGCCTCGTCTCAAGCCCCCCCTTGGAGGCTGAAATCACCCCAGGCACTGTGA
GGGCTTCTCCAGGGGGCACCCTTTGAGCTGTGGGTCTGATCACCCCAAGTCCCGCCC
GGAGGAGAGGCACAGCCAGGGCGTGTGGTTTAATGTTTGCCCCCTTCGGGGCTGGA
GGTCTCAGTGTTTCTAGATTCCAGACCCTGCTGCCAGAGAGACCTGCTGCCGGAGAG
AAGGGGAGGAGGACTCCAGCTGGGCTCGGTCCCCCACAGTCAGGGACCCCCATAAA
GGACACCCCCTTCTCTCTAGAAAGAGCTGGGCTCTCAGCTATTTCTAGTTGCTTCCCA
GAAGCCGAGGAGCAGAAGGAGCTGTGAGAGCTTTGCAGAAACGCCCTTGTCCCCGC
CCTCCTGAGCTATGAATGCCGTACAGAGCAGAGGCTGGGGCATTGGCAAGATCACA
GGTTGATGCTGCACAGCCCCATTGACACAAACCCTCAAAGCAGACGTGAGAGGGAC
GGTTCACAAAGCTCGGACCTGCCGTGGAGGGTGCCCGGCAGACGTGGCGTGAGAGG
GACGGCTCACGAGGCTTGGACCTGCTGTGGAGGGTGCCCAGCAGACGTGGTGTGAG
AGGAACGGCTCACGAGACTTGGACCTGGTGGAGGGTGCCCAGCAGACGTGGTGTGA
GAGGGACGGCTCACAGGGCTTGGACCTGCCATGGAGGGTGCCCGGCAGACGTGGTG
TGAGAGGGATGGTTCACAGGGCTTGGACCTGCCATGGAGGGTGCCCGGCAGATGTG
GTGGGAGAGAGATGGCTCATGAGGCTTGGACCTGCCGTGGAGGGTGCCCAGCAGAC
GTGGTATGAGAGGGATGGCTCACGAGGCTTGGACCTGGTGGAGGGTGCCCGGCAGA
CGTGTGAGAGGGACGGTTCACAAGGCTTGGACCTGCCATGGAGGGTGCCCAGCAGA
CGTGGTGTGAGAGGGACAGCTCACGAGGCTTGGACCTGCTGTGGAGGGTGCCCAGC
AGGGGGCTGAGCTCTGAGGGGTGGGTGCTCAGTGCACGGGTGCCCCCAGTGTCCTCT
GATCCTGTCCGGTGCCTCCCCCAACCCCCACACCCATGCAGAACTCCCAGGTCACAT
GCACGTATGTCCAGGGCATGGGGGTGGCGTGAAGAGGCCTGGTCAGGGCCTTTAGG
GGCTGCAGGACGGAATGGCCGCCTGGGGAGCCTGTGTGGCTGTGCCGGGCAGCCAT
CCTGCATTCCCACCCAGCGCGCAGTCTCCACCTCGGCCCCAGCAAAGCGCTAAGCAG
CCGGAGAGACAGCCAGGGCGGCTTCCTGAAGGATGTGGGATGGTGGACTCCGGGGT
CGAGGGAATACGCAGGTTCCTGTCCCTCCGGGAGACCTAGAGAAGCTGCACACCCA
GGAGCTTTCCATGACCCGGGAGCATGAGTGAATGGGGGTTCCAGTTTGCTGAACTTT
GCCGTCTTGTAAGGGTGGGGGCTGACGGCCGACCCTGGGAGGAGGTGACATCGCCG
GGGGAGGTTGTGGGCAACGGTGGAGGAGGAGAGACGGGAGGGGACCATTTGGGAT
GGAGGGGCCTCTTCAGAGTTTTAAAAGGCGTTTGTGGGGTGGAGTTGAGTGTGCTCT
GGGCTTGGACACTTGCCGTGGTGCCCCTGGCTGGCCGAGGAGACTGGCTCTGGCCAG
GGGCCCCGTCCTGAGAGGTCCTCAGCGTCTGACTCTCGGCCAGGCGCCAGCAAGGA
GGGGCCGGTCCCCGGGGCTACCAGGCAGGCACGTGCACATCGCCATCGCCACACGC
CAACTCCGCCTGGGTTTTACAAAGTCGTTGCCTTAATGCATGTGGACAGGAACTCCC
TGAGGTCGCCCCATGCCCCCTGGCTGTGCCAGGTACGGACGCCCTGGACCCTGCGAA
CAGGTGGGGGGGGCGAGGGGCCCAAGGGACGGGCTCCAGAGACACGCGCAGGGCA
GGAGGGGTCTCACGGAGGGGTCTCGCACTGAGGCGCCCAGAGCTGGTGGTCCCGCT
GGACGCCATCCCTCTGCCCGGGATCCACACGGCCCACGTGTGCCCGCCATGCCCGCG
CCCCACGCCATTGCAGTCTGCCATCCTCTGGCCGTGACGGTGGCTGCAGCTTCCCCA
TTTGCGCCGTTGCCTCTGGCTGTCTGCACTTTTGTTCATGCTCCAAAGAACATTTCAT
AATGCCTTCAGTACCGACGTACACTTCTGACCATTTTGTATGTGTCCTTGTGCCGTAG
TGACCAGGCCTTTTTTTGGTGGATGTGTTACCCCGCACACTTCAATCTCAACTTTGTG
CACCGTCCATTTTCTAGGGATAGACGCCCAGGGAATGAACTCTAGTTTTCTAACAGA
TTAGCTGAGATATTAACTTACTCACACGGACAGGTTGATGCCAGAGCCGTAAGAATG
CGCCAGTGCGGGTTTGCGGGGGACTTCGGGTGTGGGGTCCTGCGGCCGCGATGGCCG
TGGAAGGTTCTGGGGATCCCTGCTGCCACGGGGACGAGTTCGGACGCCAGGTGGAC
CTGTGCACTCAGTAAAACGCAGTGATTCAACCTGGA
(Source: NCBI Reference Sequence: NM_172108.5).

In various embodiments, KCNQ2 miRNA transcript comprises the sequence provided as SEQ ID NO: 3037.

(SEQ ID NO: 3037)
AGGCGCCGAGGTGCGCGCGGAGCGAGGTGGCCGCAGCGTCTCCGCGCGCGGCCCAA
GCCCGGCAGGAGTGCGGAACCGCCGCCTCGGCCATGCGGCTCCCGGCCGGGGGGCC
TGGGCTGGGGCCCGCGCCGCCCCCCGCGCTCCGCCCCCGCTGAGCCTGAGCCCGACC
CGGGGCGCCTCCCGCCAGGCACCATGGTGCAGAAGTCGCGCAACGGCGGCGTATAC
CCCGGCCCGAGCGGGGAGAAGAAGCTGAAGGTGGGCTTCGTGGGGCTGGACCCCGG
CGCGCCCGACTCCACCCGGGACGGGGCGCTGCTGATCGCCGGCTCCGAGGCCCCCA
AGCGCGGCAGCATCCTCAGCAAACCTCGCGCGGGCGGCGCGGGCGCCGGGAAGCCC
CCCAAGCGCAACGCCTTCTACCGCAAGCTGCAGAATTTCCTCTACAACGTGCTGGAG
CGGCCGCGCGGCTGGGCGTTCATCTACCACGCCTACGTGTTCCTCCTGGTTTTCTCCT
GCCTCGTGCTGTCTGTGTTTTCCACCATCAAGGAGTATGAGAAGAGCTCGGAGGGGG
CCCTCTACATCCTGGAAATCGTGACTATCGTGGTGTTTGGCGTGGAGTACTTCGTGCG
GATCTGGGCCGCAGGCTGCTGCTGCCGGTACCGTGGCTGGAGGGGGCGGCTCAAGTT
TGCCCGGAAACCGTTCTGTGTGATTGACATCATGGTGCTCATCGCCTCCATTGCGGTG
CTGGCCGCCGGCTCCCAGGGCAACGTCTTTGCCACATCTGCGCTCCGGAGCCTGCGC
TTCCTGCAGATTCTGCGGATGATCCGCATGGACCGGCGGGGAGGCACCTGGAAGCTG
CTGGGCTCTGTGGTCTATGCCCACAGCAAGGAGCTGGTCACTGCCTGGTACATCGGC
TTCCTTTGTCTCATCCTGGCCTCGTTCCTGGTGTACTTGGCAGAGAAGGGGGAGAAC
GACCACTTTGACACCTACGCGGATGCACTCTGGTGGGGCCTGATCACGCTGACCACC
ATTGGCTACGGGGACAAGTACCCCCAGACCTGGAACGGCAGGCTCCTTGCGGCAAC
CTTCACCCTCATCGGTGTCTCCTTCTTCGCGCTGCCTGCAGGCATCTTGGGGTCTGGG
TTTGCCCTGAAGGTTCAGGAGCAGCACAGGCAGAAGCACTTTGAGAAGAGGCGGAA
CCCGGCAGCAGGCCTGATCCAGTCGGCCTGGAGATTCTACGCCACCAACCTCTCGCG
CACAGACCTGCACTCCACGTGGCAGTACTACGAGCGAACGGTCACCGTGCCCATGTA
CAGGTACCGCCGCCGGGCACCTGCCACCAAGCAACTGTTTCATTTTTTATTTTCCATT
TGTTCTTAAACCCCACTTTTTGTTGTTCATTATTTTGATTGATTTTTTTTCTTTAAAATG
TATTTTTCACAAAGGA
(Source: NCBI Reference Sequence: NM_172109.3).

In various embodiments, a KCNQ2 miRNA transcript comprises the sequence provided as SEQ ID NO: 3038.

(SEQ ID NO: 3038)
AGCGTCTCCGCGCGCGGCCCAAGCCCGGCAGGAGTGCGGAACCGCCGCCTCGGCCA
TGCGGCTCCCGGCCGGGGGGCCTGGGCTGGGGCCCGCGCCGCCCCCCGCGCTCCGCC
CCCGCTGAGCCTGAGCCCGACCCGGGGCGCCTCCCGCCAGGCACCATGGTGCAGAA
GTCGCGCAACGGCGGCGTATACCCCGGCCCGAGCGGGGAGAAGAAGCTGAAGGTGG
GCTTCGTGGGGCTGGACCCCGGCGCGCCCGACTCCACCCGGGACGGGGCGCTGCTG
ATCGCCGGCTCCGAGGCCCCCAAGCGCGGCAGCATCCTCAGCAAACCTCGCGCGGG
CGGCGCGGGCGCCGGGAAGCCCCCCAAGCGCAACGCCTTCTACCGCAAGCTGCAGA
ATTTCCTCTACAACGTGCTGGAGCGGCCGCGCGGCTGGGCGTTCATCTACCACGCCT
ACGTGTTCCTCCTGGTTTTCTCCTGCCTCGTGCTGTCTGTGTTTTCCACCATCAAGGA
GTATGAGAAGAGCTCGGAGGGGGCCCTCTACATCCTGGAAATCGTGACTATCGTGGT
GTTTGGCGTGGAGTACTTCGTGCGGATCTGGGCCGCAGGCTGCTGCTGCCGGTACCG
TGGCTGGAGGGGGCGGCTCAAGTTTGCCCGGAAACCGTTCTGTGTGATTGACATCAT
GGTGCTCATCGCCTCCATTGCGGTGCTGGCCGCCGGCTCCCAGGGCAACGTCTTTGC
CACATCTGCGCTCCGGAGCCTGCGCTTCCTGCAGATTCTGCGGATGATCCGCATGGA
CCGGCGGGGAGGCACCTGGAAGCTGCTGGGCTCTGTGGTCTATGCCCACAGCAAGG
AGCTGGTCACTGCCTGGTACATCGGCTTCCTTTGTCTCATCCTGGCCTCGTTCCTGGT
GTACTTGGCAGAGAAGGGGGAGAACGACCACTTTGACACCTACGCGGATGCACTCT
GGTGGGGCCTGATCACGCTGACCACCATTGGCTACGGGGACAAGTACCCCCAGACC
TGGAACGGCAGGCTCCTTGCGGCAACCTTCACCCTCATCGGTGTCTCCTTCTTCGCGC
TGCCTGCAGGCATCTTGGGGTCTGGGTTTGCCCTGAAGGTTCAGGAGCAGCACAGGC
AGAAGCACTTTGAGAAGAGGCGGAACCCGGCAGCAGGCCTGATCCAGTCGGCCTGG
AGATTCTACGCCACCAACCTCTCGCGCACAGACCTGCACTCCACGTGGCAGTACTAC
GAGCGAACGGTCACCGTGCCCATGTACAGACTTATCCCCCCGCTGAACCAGCTGGAG
CTGCTGAGGAACCTCAAGAGTAAATCTGGACTCGCTTTCAGGAAGGACCCCCCGCCG
GAGCCGTCTCCAAGCCAGAAGGTCAGTTTGAAAGATCGTGTCTTCTCCAGCCCCCGA
GGCGTGGCTGCCAAGGGGAAGGGGTCCCCGCAGGCCCAGACTGTGAGGCGGTCACC
CAGCGCCGACCAGAGCCTCGAGGACAGCCCCAGCAAGGTGCCCAAGAGCTGGAGCT
TCGGGGACCGCAGCCGGGCACGCCAGGCTTTCCGCATCAAGGGTGCCGCGTCACGG
CAGAACTCAGAAGAAGCAAGCCTCCCCGGAGAGGACATTGTGGATGACAAGAGCTG
CCCCTGCGAGTTTGTGACCGAGGACCTGACCCCGGGCCTCAAAGTCAGCATCAGAGC
CGTGTGTGTCATGCGGTTCCTGGTGTCCAAGCGGAAGTTCAAGGAGAGCCTGCGGCC
CTACGACGTGATGGACGTCATCGAGCAGTACTCAGCCGGCCACCTGGACATGCTGTC
CCGAATTAAGAGCCTGCAGTCCAGGATAGATATGATTGTGGGTCCCCCGCCCCCTTC
AACTCCCCGGCACAAGAAGTACCCCACCAAAGGACCCACGGCCCCTCCGAGAGAGT
CACCCCAGTACTCACCTAGAGTGGACCAGATCGTGGGGGGGGCCCAGCGATCACG
GACAAGGACCGCACCAAGGGCCCGGCCGAGGCGGAGCTGCCCGAGGACCCCAGCAT
GATGGGACGGCTCGGGAAGGTGGAGAAGCAGGTCTTGTCCATGGAGAAGAAGCTGG
ACTTCCTGGTGAATATCTACATGCAGCGGATGGGCATCCCCCCGACAGAGACCGAGG
CCTACTTTGGGGCCAAAGAGCCGGAGCCGGCGCCGCCGTACCACAGCCCGGAAGAC
AGCCGGGAGCATGTCGACAGGCACGGCTGCATTGTCAAGATCGTGCGCTCCAGCAG
CTCCACGGGCCAGAAGAACTTCTCGGCGCCCCCGGCCGCGCCCCCTGTCCAGTGTCC
GCCCTCCACCTCCTGGCAGCCACAGAGCCACCCGCGCCAGGGCCACGGCACCTCCCC
CGTGGGGGACCACGGCTCCCTGGTGCGCATCCCGCCGCCGCCTGCCCACGAGCGGTC
GCTGTCCGCCTACGGCGGGGGCAACCGCGCCAGCATGGAGTTCCTGCGGCAGGAGG
ACACCCCGGGCTGCAGGCCCCCCGAGGGGAACCTGCGGGACAGCGACACGTCCATC
TCCATCCCGTCCGTGGACCACGAGGAGCTGGAGCGTTCCTTCAGCGGCTTCAGCATC
TCCCAGTCCAAGGAGAACCTGGATGCTCTCAACAGCTGCTACGCGGCCGTGGCGCCT
TGTGCCAAAGTCAGGCCCTACATTGCGGAGGGAGAGTCAGACACCGACTCCGACCT
CTGTACCCCGTGCGGGCCCCCGCCACGCTCGGCCACCGGCGAGGGTCCCTTTGGTGA
CGTGGGCTGGGCCGGGCCCAGGAAGTGAGGCGGCGCTGGGCCAGTGGACCCGCCCG
CGGCCCTCCTCAGCACGGTGCCTCCGAGGTTTTGAGGCGGGAACCCTCTGGGGCCCT
TTTCTTACAGTAACTGAGTGTGGCGGGAAGGGTGGGCCCTGGAGGGGCCCATGTGG
GCTGAAGGATGGGGGCTCCTGGCAGTGACCTTTTACAAAAGTTATTTTCCAACAGGG
GCTGGAGGGCTGGGCAGGGCCCTGTGGCTCCAGGAGCAGCGTGCAGGAGCAAGGCT
GCCCTGTCCACTCTGCTCAGGGCCGCGGCCGACATCAGCCCGGTGTGAGGAGGGGC
GGGAGTGATGACGGGGTGTTGCCAGCGTGGCAACAGGCGGGGGGTTGTCTCAGCCG
AGCCCAGGGGAGGCACAAAGGGCAGGCCTGTTCCCTGAGGACCTGCGCAAAGGGCG
GGCCTGTTTGGTGAGGACCTGCGGCCTTGGGTCCCGGTGGGGTTTCCGGGCAGCTAC
AGGCGGGTGTGGCCGGCCGCTGTGCGTGGCCTCTGCCTTCACACCTGACCTGCCCGG
CGGGCTTTCCTGTTCCCCACCTCAGGGGCGCCCAAATACAGAGCTATTGGTTGGCGT
CTTCTCCCTGTACCTTCTGGGATCTGAGGGCTCTTTCCATGGAAGCCAGCCCCGAGGT
GGAGACCTTCGCCTGCAGCCGAGGAGCGGGTGGGGCCTGGGAACCAAACTGGAGCC
AGAGTGGACGTCCAGCCCTCTGGTCTTGGCCTCCAGAGGGAGGGCCTGGCTCACGGT
GGGGCCAGGGAGCCGGCTCCAAAGGGTCTTCAAAAAGGGGGTCCTTGGGGGCTCCA
GCTGCCTCGCCCTGGCCTTTCTGTGGGTGCGTGAGAGCCAGCAGCACCCCAGCCTTG
GAGACCGGGGGGGCAGGACCCCAAGTCCTCCCCTCTCTCCTGACTGCCCTGGCCGGG
TGCCGGCACTGCGAGACCCACCTGGTGAGCAGGCCTCACAGTTCTTAGCCAGGGCCC
CACCTCGCCTGTGTCCCACCAGTGCCCCGACAGACCTGGGGCAGGGCTGGGCCATGA
TGCAGCGGGCCAGGATAGCCTCCACCGTCAGCACAGGGCCGCCCTCCCCGCCTTTCC
GGAGGAAACCACTCCCACCTCAGCCCAGCTGTGCGCCCTCCCTAGCTCTCCTGCCCC
CTGGAGCTGATGGCCCCTTCTCCACTGACCGATTCCTTAGCGGGGCCTCTTGGGGTCT
CGGGCCTCGGGTGCACCGTCCCATGCCCGTCCTGTTGTGGGCACCGTGGCCCTTGGG
GCAGGCGGCTCTAATGCGGGAGCGAGTCCCTAGCTCCAGACTTAAGAACCAGACCC
CGGGAGCATCTGGCATTTGGCGTGACGGCGTCGCAGGCGGGCCTGGGCTCCCTGGA
GAGTGGCCTCCCTGGGAGTGAGCAGGGCTGGGGTCGTGGGCGCAAATACTCCTGCA
GAGCAAGTGCAGGGGAGTCCTGGGCCCGTTTCTCCTCCACCTGCGTTTTCAGTGCAC
TTGGCTTGGCTGGGAGGTCCTGAGGCCCTGAGGCCAGCAGGGGAACCAGTCCTGAG
GGAGAGGACTTTGAAAGCAGCATTTGAGGGTCGTACGCCCCTGGCTGGTGGGGGTC
CTGGCGCTCAGGGTGTTCGGGGAGCCATGTCTGGCGTCCATTGTGGGGAGCTGCTGC
CCTGGCCTCTCTGCCTACCCCCAGCCCGGCCAGGGCACTCCCAGGCCCTGTCGCCAT
TGAGGTGCCTCCGCTGGGCTGTCTCCTCACCCCTCCCTGTGCTGGAGCCTGTCCCAAA
AAGGTGCCAACTGGGAGGCCTCGGAAGCCACTGTCCAGGCTCCCACTGCCTGTCTGC
TCTGTTCCCAAAGGCAGCGTGTGTGGCCTCGGGCCCTGCGGTGGCATGAAGCATCCC
TTCTGGTGTGGGCATCGCTACGTGTTTTGGGGGCAGCGTTTCACGGCGGTGCCCTTGC
TGTCTCCCTTGGGCTGGCTCGAGCCTGGGGTCCATGTCCCTTTGCCGTCCCGTCATGG
GGCAGGGAATCCATAGCGGGGCCCACAGGCAGGGGTATGAGTGCGTCCCACCCAAC
GCAGCACCAGCCCCGGCCACCGCTCCCCGTGTCCCCAGTTCCGTCTCAGCTACCTGG
ACTCCAGGACCCTGGAGAAGGGAGACCTGGCAGTGGAGGGAGGCTGTGCTGTGTGT
CCCCCTGCAGGTGTGACCCCGCCTGCTCTTTCCTCCCCCGCCAGGTGTGGCCCCGCCT
GCTCTTTCCTCCCCCACCAGTATGGCCCCACCTGCTCTTTCCTCCCCCCCCAAGGTGT
GGCCCCACCTGTTCTTTCCTCCCCTGCCGAGGTGTGACCCCACCTGCTCTTTCCTCCC
TCCCAGTATGGCCCCACCTGCTCTTTCCTCCCCCGAGGTGAGGCCCCGCCTGCTCTTT
CCTCCCATGGGGCCGCTGAGGCATGAGCACCTGGGCACAGGTTGGGGCTCTGCAGG
ATGAGGAAGACAGGCCAATCCCTTCCCTCCCAGAAGCTGGCCGCCCAGCAGGAGGG
ACTGAGGCCAGACTCATGTCCAGCAAGGAACGTGTGGTGTGTCCCCTGGGAAGTCTC
TGGGCCCTGGGAAGAGGGAAGGTGCACGTCCTGGGATGGTTGCGGGGCCCTGTTTTG
GGAGACAAAGGGGTAGAGGGTCTGTCTTGGGCCCCCCCAGACTCTAGCCTGAGCAG
TGCAGCCACCTACTGCCCCACCTCAGAGAAGTGCAGCGGGAAGGAGGCTGGAGGTG
GTGCGGCGCTGCCTCGGGTGTCTGCGTGAATGAGCGTGGCCAAGGACCAGTGCCACC
TCATGGCAAAGAGCTCCCGCAGTGTTTGTTAGAGTGCACATCCCTACGTGCCCACTG
GCACACACACGTGCTCACATACATGTCCGCATACAGGCGTACACATGCACGCTTGCA
CACATGCACACAGACCACATAGCACACATGTGCACTGACCACACCTGTATAGACCAT
GCACAGTACACATACGTGCATACACATGCCTGCATACAGGCATACACATGCACGCTT
ACATGTACACGTGCACAGATCACACACATGCACACACGTGTAGCTCACACACAGTAT
ACACATACACAAGTGCACAGACCACACACAGCACTAACACATGCACACACAAAGTG
CATAGGCCACACAGCACATGCACACAGGTGCACAGACCACACAGCACACACAAGTG
CACAGAGCACACTGCACACATGCACACACACACGCGTGCATGCACACTCCTCGCACT
TCCAGCCTTGGAGCCCTTCTGTCTCTGGTCTTTCTCTTTGACCCTGCTGAGTGTAAGC
TGCCTGGGGAGGGGCTACAAGGAGTAATTGTGGCTTTAGGGGTCGTGGTGATGCTGG
AATGTCAAGCGCCGTCGTGGGGTATCCGACTGTCCGGGCTCCTGGTCCGCAGTGGCA
GAGCGCCAGGCAGAGCCCAATCAGGGTCTCGTGCTGCCCTTCCCTCCCACAGCCTGG
CAGCCATCCAGAGGAGGGGCTCTACCAGATGCCAAGGTGCCCCGGTGTCTGTATGG
GTGTCCGGTTGGGTCCTGTGTTTGGTCTGCCCTGGAGGTGCTGGGCCCTCCTGGGATG
GGTGGCTCAGCCTCGAATCCCAGGCCCCAGCCCAGGCAGGTGCTGCTGCCTGTTGTG
GTTTCCTGGCCCAGCTTCTCCTTCTCCCTCTGCATAAAATCACAGTCCGTGAGTCTTC
CAGCTGCCACCACGGCTGGGACACGCTGGGGGAGGGCTCCTCCCATGCCTCCTGCAC
ACAGCCGTCTGAGCAGGGCAGGTGCCCAACACCCCCCACCGGAGGACACGCTGCCC
CTCAGCGATGCCCCTACCTTTTGGGGGGCCTCGTCTCAAGCCCCCCCTTGGAGGCTG
AAATCACCCCAGGCACTGTGAGGGCTTCTCCAGGGGGCACCCTTTGAGCTGTGGGTC
TGATCACCCCAAGTCCCGCCCGGAGGAGAGGCACAGCCAGGGCGTGTGGTTTAATG
TTTGCCCCCTTCGGGGCTGGAGGTCTCAGTGTTTCTAGATTCCAGACCCTGCTGCCAG
AGAGACCTGCTGCCGGAGAGAAGGGGAGGAGGACTCCAGCTGGGCTCGGTCCCCCA
CAGTCAGGGACCCCCATAAAGGACACCCCCTTCTCTCTAGAAAGAGCTGGGCTCTCA
GCTATTTCTAGTTGCTTCCCAGAAGCCGAGGAGCAGAAGGAGCTGTGAGAGCTTTGC
AGAAACGCCCTTGTCCCCGCCCTCCTGAGCTATGAATGCCGTACAGAGCAGAGGCTG
GGGCATTGGCAAGATCACAGGTTGATGCTGCACAGCCCCATTGACACAAACCCTCAA
AGCAGACGTGAGAGGGACGGTTCACAAAGCTCGGACCTGCCGTGGAGGGTGCCCGG
CAGACGTGGCGTGAGAGGGACGGCTCACGAGGCTTGGACCTGCTGTGGAGGGTGCC
CAGCAGACGTGGTGTGAGAGGAACGGCTCACGAGACTTGGACCTGGTGGAGGGTGC
CCAGCAGACGTGGTGTGAGAGGGACGGCTCACAGGGCTTGGACCTGCCATGGAGGG
TGCCCGGCAGACGTGGTGTGAGAGGGATGGTTCACAGGGCTTGGACCTGCCATGGA
GGGTGCCCGGCAGATGTGGTGGGAGAGAGATGGCTCATGAGGCTTGGACCTGCCGT
GGAGGGTGCCCAGCAGACGTGGTATGAGAGGGATGGCTCACGAGGCTTGGACCTGG
TGGAGGGTGCCCGGCAGACGTGTGAGAGGGACGGTTCACAAGGCTTGGACCTGCCA
TGGAGGGTGCCCAGCAGACGTGGTGTGAGAGGGACAGCTCACGAGGCTTGGACCTG
CTGTGGAGGGTGCCCAGCAGGGGGCTGAGCTCTGAGGGGTGGGTGCTCAGTGCACG
GGTGCCCCCAGTGTCCTCTGATCCTGTCCGGTGCCTCCCCCAACCCCCACACCCATGC
AGAACTCCCAGGTCACATGCACGTATGTCCAGGGCATGGGGGTGGCGTGAAGAGGC
CTGGTCAGGGCCTTTAGGGGCTGCAGGACGGAATGGCCGCCTGGGGAGCCTGTGTG
GCTGTGCCGGGCAGCCATCCTGCATTCCCACCCAGCGCGCAGTCTCCACCTCGGCCC
CAGCAAAGCGCTAAGCAGCCGGAGAGACAGCCAGGGCGGCTTCCTGAAGGATGTGG
GATGGTGGACTCCGGGGTCGAGGGAATACGCAGGTTCCTGTCCCTCCGGGAGACCTA
GAGAAGCTGCACACCCAGGAGCTTTCCATGACCCGGGAGCATGAGTGAATGGGGGT
TCCAGTTTGCTGAACTTTGCCGTCTTGTAAGGGTGGGGGCTGACGGCCGACCCTGGG
AGGAGGTGACATCGCCGGGGGAGGTTGTGGGCAACGGTGGAGGAGGAGAGACGGG
AGGGGACCATTTGGGATGGAGGGGCCTCTTCAGAGTTTTAAAAGGCGTTTGTGGGGT
GGAGTTGAGTGTGCTCTGGGCTTGGACACTTGCCGTGGTGCCCCTGGCTGGCCGAGG
AGACTGGCTCTGGCCAGGGGCCCCGTCCTGAGAGGTCCTCAGCGTCTGACTCTCGGC
CAGGCGCCAGCAAGGAGGGGCCGGTCCCCGGGGCTACCAGGCAGGCACGTGCACAT
CGCCATCGCCACACGCCAACTCCGCCTGGGTTTTACAAAGTCGTTGCCTTAATGCAT
GTGGACAGGAACTCCCTGAGGTCGCCCCATGCCCCCTGGCTGTGCCAGGTACGGACG
CCCTGGACCCTGCGAACAGGTGGGGGGGGCGAGGGGCCCAAGGGACGGGCTCCAGA
GACACGCGCAGGGCAGGAGGGGTCTCACGGAGGGGTCTCGCACTGAGGCGCCCAGA
GCTGGTGGTCCCGCTGGACGCCATCCCTCTGCCCGGGATCCACACGGCCCACGTGTG
CCCGCCATGCCCGCGCCCCACGCCATTGCAGTCTGCCATCCTCTGGCCGTGACGGTG
GCTGCAGCTTCCCCATTTGCGCCGTTGCCTCTGGCTGTCTGCACTTTTGTTCATGCTCC
AAAGAACATTTCATAATGCCTTCAGTACCGACGTACACTTCTGACCATTTTGTATGTG
TCCTTGTGCCGTAGTGACCAGGCCTTTTTTTGGTGGATGTGTTACCCCGCACACTTCA
ATCTCAACTTTGTGCACCGTCCATTTTCTAGGGATAGACGCCCAGGGAATGAACTCT
AGTTTTCTAACAGATTAGCTGAGATATTAACTTACTCACACGGACAGGTTGATGCCA
GAGCCGTAAGAATGCGCCAGTGCGGGTTTGCGGGGGACTTCGGGTGTGGGGTCCTG
CGGCCGCGATGGCCGTGGAAGGTTCTGGGGATCCCTGCTGCCACGGGGACGAGTTC
GGACGCCAGGTGGACCTGTGCACTCAGTAAAACGCAGTGATTCAACCTGGA
(Source: NCBI Reference Sequence: XM_011528811.2))

In various embodiments, a KCNQ2 miRNA transcript comprises the sequence provided as SEQ ID NO: 3039.

(SEQ ID NO: 3039)
AGCGTCTCCGCGCGCGGCCCAAGCCCGGCAGGAGTGCGGAACCGCCGCCTCGGCCA
TGCGGCTCCCGGCCGGGGGGCCTGGGCTGGGGCCCGCGCCGCCCCCCGCGCTCCGCC
CCCGCTGAGCCTGAGCCCGACCCGGGGCGCCTCCCGCCAGGCACCATGGTGCAGAA
GTCGCGCAACGGCGGCGTATACCCCGGCCCGAGCGGGGAGAAGAAGCTGAAGGTGG
GCTTCGTGGGGCTGGACCCCGGCGCGCCCGACTCCACCCGGGACGGGGCGCTGCTG
ATCGCCGGCTCCGAGGCCCCCAAGCGCGGCAGCATCCTCAGCAAACCTCGCGCGGG
CGGCGCGGGCGCCGGGAAGCCCCCCAAGCGCAACGCCTTCTACCGCAAGCTGCAGA
ATTTCCTCTACAACGTGCTGGAGCGGCCGCGCGGCTGGGCGTTCATCTACCACGCCT
ACGTGTTCCTCCTGGTTTTCTCCTGCCTCGTGCTGTCTGTGTTTTCCACCATCAAGGA
GTATGAGAAGAGCTCGGAGGGGGCCCTCTACATCCTGGAAATCGTGACTATCGTGGT
GTTTGGCGTGGAGTACTTCGTGCGGATCTGGGCCGCAGGCTGCTGCTGCCGGTACCG
TGGCTGGAGGGGGCGGCTCAAGTTTGCCCGGAAACCGTTCTGTGTGATTGACATCAT
GGTGCTCATCGCCTCCATTGCGGTGCTGGCCGCCGGCTCCCAGGGCAACGTCTTTGC
CACATCTGCGCTCCGGAGCCTGCGCTTCCTGCAGATTCTGCGGATGATCCGCATGGA
CCGGCGGGGAGGCACCTGGAAGCTGCTGGGCTCTGTGGTCTATGCCCACAGCAAGG
AGCTGGTCACTGCCTGGTACATCGGCTTCCTTTGTCTCATCCTGGCCTCGTTCCTGGT
GTACTTGGCAGAGAAGGGGGAGAACGACCACTTTGACACCTACGCGGATGCACTCT
GGTGGGGCCTGATCACGCTGACCACCATTGGCTACGGGGACAAGTACCCCCAGACC
TGGAACGGCAGGCTCCTTGCGGCAACCTTCACCCTCATCGGTGTCTCCTTCTTCGCGC
TGCCTGCAGGCATCTTGGGGTCTGGGTTTGCCCTGAAGGTTCAGGAGCAGCACAGGC
AGAAGCACTTTGAGAAGAGGCGGAACCCGGCAGCAGGCCTGATCCAGTCGGCCTGG
AGATTCTACGCCACCAACCTCTCGCGCACAGACCTGCACTCCACGTGGCAGTACTAC
GAGCGAACGGTCACCGTGCCCATGTACAGTTCGCAAACTCAAACCTACGGGGCCTCC
AGACTTATCCCCCCGCTGAACCAGCTGGAGCTGCTGAGGAACCTCAAGAGTAAATCT
GGACTCGCTTTCAGGAAGGACCCCCCGCCGGAGCCGTCTCCAAGCCAGAAGGTCAG
TTTGAAAGATCGTGTCTTCTCCAGCCCCCGAGGCGTGGCTGCCAAGGGGAAGGGGTC
CCCGCAGGCCCAGACTGTGAGGCGGTCACCCAGCGCCGACCAGAGCCTCGAGGACA
GCCCCAGCAAGGTGCCCAAGAGCTGGAGCTTCGGGGACCGCAGCCGGGCACGCCAG
GCTTTCCGCATCAAGGGTGCCGCGTCACGGCAGAACTCAGAAGCAAGCCTCCCCGG
AGAGGACATTGTGGATGACAAGAGCTGCCCCTGCGAGTTTGTGACCGAGGACCTGA
CCCCGGGCCTCAAAGTCAGCATCAGAGCCGTGTGTGTCATGCGGTTCCTGGTGTCCA
AGCGGAAGTTCAAGGAGAGCCTGCGGCCCTACGACGTGATGGACGTCATCGAGCAG
TACTCAGCCGGCCACCTGGACATGCTGTCCCGAATTAAGAGCCTGCAGTCCAGAGTG
GACCAGATCGTGGGGCGGGGCCCAGCGATCACGGACAAGGACCGCACCAAGGGCCC
GGCCGAGGCGGAGCTGCCCGAGGACCCCAGCATGATGGGACGGCTCGGGAAGGTGG
AGAAGCAGGTCTTGTCCATGGAGAAGAAGCTGGACTTCCTGGTGAATATCTACATGC
AGCGGATGGGCATCCCCCCGACAGAGACCGAGGCCTACTTTGGGGCCAAAGAGCCG
GAGCCGGCGCCGCCGTACCACAGCCCGGAAGACAGCCGGGAGCATGTCGACAGGCA
CGGCTGCATTGTCAAGATCGTGCGCTCCAGCAGCTCCACGGGCCAGAAGAACTTCTC
GGCGCCCCCGGCCGCGCCCCCTGTCCAGTGTCCGCCCTCCACCTCCTGGCAGCCACA
GAGCCACCCGCGCCAGGGCCACGGCACCTCCCCCGTGGGGGACCACGGCTCCCTGG
TGCGCATCCCGCCGCCGCCTGCCCACGAGCGGTCGCTGTCCGCCTACGGCGGGGGCA
ACCGCGCCAGCATGGAGTTCCTGCGGCAGGAGGACACCCCGGGCTGCAGGCCCCCC
GAGGGGAACCTGCGGGACAGCGACACGTCCATCTCCATCCCGTCCGTGGACCACGA
GGAGCTGGAGCGTTCCTTCAGCGGCTTCAGCATCTCCCAGTCCAAGGAGAACCTGGA
TGCTCTCAACAGCTGCTACGCGGCCGTGGCGCCTTGTGCCAAAGTCAGGCCCTACAT
TGCGGAGGGAGAGTCAGACACCGACTCCGACCTCTGTACCCCGTGCGGGCCCCCGC
CACGCTCGGCCACCGGCGAGGGTCCCTTTGGTGACGTGGGCTGGGCCGGGCCCAGG
AAGTGAGGCGGCGCTGGGCCAGTGGACCCGCCCGCGGCCCTCCTCAGCACGGTGCC
TCCGAGGTTTTGAGGCGGGAACCCTCTGGGGCCCTTTTCTTACAGTAACTGAGTGTG
GCGGGAAGGGTGGGCCCTGGAGGGGCCCATGTGGGCTGAAGGATGGGGGCTCCTGG
CAGTGACCTTTTACAAAAGTTATTTTCCAACAGGGGCTGGAGGGCTGGGCAGGGCCC
TGTGGCTCCAGGAGCAGCGTGCAGGAGCAAGGCTGCCCTGTCCACTCTGCTCAGGGC
CGCGGCCGACATCAGCCCGGTGTGAGGAGGGGGGGGAGTGATGACGGGGTGTTGCC
AGCGTGGCAACAGGCGGGGGGTTGTCTCAGCCGAGCCCAGGGGAGGCACAAAGGGC
AGGCCTGTTCCCTGAGGACCTGCGCAAAGGGCGGGCCTGTTTGGTGAGGACCTGCGG
CCTTGGGTCCCGGTGGGGTTTCCGGGCAGCTACAGGCGGGTGTGGCCGGCCGCTGTG
CGTGGCCTCTGCCTTCACACCTGACCTGCCCGGCGGGCTTTCCTGTTCCCCACCTCAG
GGGCGCCCAAATACAGAGCTATTGGTTGGCGTCTTCTCCCTGTACCTTCTGGGATCTG
AGGGCTCTTTCCATGGAAGCCAGCCCCGAGGTGGAGACCTTCGCCTGCAGCCGAGG
AGCGGGTGGGGCCTGGGAACCAAACTGGAGCCAGAGTGGACGTCCAGCCCTCTGGT
CTTGGCCTCCAGAGGGAGGGCCTGGCTCACGGTGGGGCCAGGGAGCCGGCTCCAAA
GGGTCTTCAAAAAGGGGGTCCTTGGGGGCTCCAGCTGCCTCGCCCTGGCCTTTCTGT
GGGTGCGTGAGAGCCAGCAGCACCCCAGCCTTGGAGACCGGGGGGGCAGGACCCCA
AGTCCTCCCCTCTCTCCTGACTGCCCTGGCCGGGTGCCGGCACTGCGAGACCCACCT
GGTGAGCAGGCCTCACAGTTCTTAGCCAGGGCCCCACCTCGCCTGTGTCCCACCAGT
GCCCCGACAGACCTGGGGCAGGGCTGGGCCATGATGCAGCGGGCCAGGATAGCCTC
CACCGTCAGCACAGGGCCGCCCTCCCCGCCTTTCCGGAGGAAACCACTCCCACCTCA
GCCCAGCTGTGCGCCCTCCCTAGCTCTCCTGCCCCCTGGAGCTGATGGCCCCTTCTCC
ACTGACCGATTCCTTAGCGGGGCCTCTTGGGGTCTCGGGCCTCGGGTGCACCGTCCC
ATGCCCGTCCTGTTGTGGGCACCGTGGCCCTTGGGGCAGGCGGCTCTAATGCGGGAG
CGAGTCCCTAGCTCCAGACTTAAGAACCAGACCCCGGGAGCATCTGGCATTTGGCGT
GACGGCGTCGCAGGCGGGCCTGGGCTCCCTGGAGAGTGGCCTCCCTGGGAGTGAGC
AGGGCTGGGGTCGTGGGCGCAAATACTCCTGCAGAGCAAGTGCAGGGGAGTCCTGG
GCCCGTTTCTCCTCCACCTGCGTTTTCAGTGCACTTGGCTTGGCTGGGAGGTCCTGAG
GCCCTGAGGCCAGCAGGGGAACCAGTCCTGAGGGAGAGGACTTTGAAAGCAGCATT
TGAGGGTCGTACGCCCCTGGCTGGTGGGGGTCCTGGCGCTCAGGGTGTTCGGGGAGC
CATGTCTGGCGTCCATTGTGGGGAGCTGCTGCCCTGGCCTCTCTGCCTACCCCCAGCC
CGGCCAGGGCACTCCCAGGCCCTGTCGCCATTGAGGTGCCTCCGCTGGGCTGTCTCC
TCACCCCTCCCTGTGCTGGAGCCTGTCCCAAAAAGGTGCCAACTGGGAGGCCTCGGA
AGCCACTGTCCAGGCTCCCACTGCCTGTCTGCTCTGTTCCCAAAGGCAGCGTGTGTG
GCCTCGGGCCCTGCGGTGGCATGAAGCATCCCTTCTGGTGTGGGCATCGCTACGTGT
TTTGGGGGCAGCGTTTCACGGCGGTGCCCTTGCTGTCTCCCTTGGGCTGGCTCGAGCC
TGGGGTCCATGTCCCTTTGCCGTCCCGTCATGGGGCAGGGAATCCATAGCGGGGCCC
ACAGGCAGGGGTATGAGTGCGTCCCACCCAACGCAGCACCAGCCCCGGCCACCGCT
CCCCGTGTCCCCAGTTCCGTCTCAGCTACCTGGACTCCAGGACCCTGGAGAAGGGAG
ACCTGGCAGTGGAGGGAGGCTGTGCTGTGTGTCCCCCTGCAGGTGTGACCCCGCCTG
CTCTTTCCTCCCCCGCCAGGTGTGGCCCCGCCTGCTCTTTCCTCCCCCACCAGTATGG
CCCCACCTGCTCTTTCCTCCCCCCCCAAGGTGTGGCCCCACCTGTTCTTTCCTCCCCT
GCCGAGGTGTGACCCCACCTGCTCTTTCCTCCCTCCCAGTATGGCCCCACCTGCTCTT
TCCTCCCCCGAGGTGAGGCCCCGCCTGCTCTTTCCTCCCATGGGGCCGCTGAGGCAT
GAGCACCTGGGCACAGGTTGGGGCTCTGCAGGATGAGGAAGACAGGCCAATCCCTT
CCCTCCCAGAAGCTGGCCGCCCAGCAGGAGGGACTGAGGCCAGACTCATGTCCAGC
AAGGAACGTGTGGTGTGTCCCCTGGGAAGTCTCTGGGCCCTGGGAAGAGGGAAGGT
GCACGTCCTGGGATGGTTGCGGGGCCCTGTTTTGGGAGACAAAGGGGTAGAGGGTC
TGTCTTGGGCCCCCCCAGACTCTAGCCTGAGCAGTGCAGCCACCTACTGCCCCACCT
CAGAGAAGTGCAGCGGGAAGGAGGCTGGAGGTGGTGCGGCGCTGCCTCGGGTGTCT
GCGTGAATGAGCGTGGCCAAGGACCAGTGCCACCTCATGGCAAAGAGCTCCCGCAG
TGTTTGTTAGAGTGCACATCCCTACGTGCCCACTGGCACACACACGTGCTCACATAC
ATGTCCGCATACAGGCGTACACATGCACGCTTGCACACATGCACACAGACCACATA
GCACACATGTGCACTGACCACACCTGTATAGACCATGCACAGTACACATACGTGCAT
ACACATGCCTGCATACAGGCATACACATGCACGCTTACATGTACACGTGCACAGATC
ACACACATGCACACACGTGTAGCTCACACACAGTATACACATACACAAGTGCACAG
ACCACACACAGCACTAACACATGCACACACAAAGTGCATAGGCCACACAGCACATG
CACACAGGTGCACAGACCACACAGCACACACAAGTGCACAGAGCACACTGCACACA
TGCACACACACACGCGTGCATGCACACTCCTCGCACTTCCAGCCTTGGAGCCCTTCT
GTCTCTGGTCTTTCTCTTTGACCCTGCTGAGTGTAAGCTGCCTGGGGAGGGGCTACAA
GGAGTAATTGTGGCTTTAGGGGTCGTGGTGATGCTGGAATGTCAAGCGCCGTCGTGG
GGTATCCGACTGTCCGGGCTCCTGGTCCGCAGTGGCAGAGCGCCAGGCAGAGCCCA
ATCAGGGTCTCGTGCTGCCCTTCCCTCCCACAGCCTGGCAGCCATCCAGAGGAGGGG
CTCTACCAGATGCCAAGGTGCCCCGGTGTCTGTATGGGTGTCCGGTTGGGTCCTGTG
TTTGGTCTGCCCTGGAGGTGCTGGGCCCTCCTGGGATGGGTGGCTCAGCCTCGAATC
CCAGGCCCCAGCCCAGGCAGGTGCTGCTGCCTGTTGTGGTTTCCTGGCCCAGCTTCT
CCTTCTCCCTCTGCATAAAATCACAGTCCGTGAGTCTTCCAGCTGCCACCACGGCTG
GGACACGCTGGGGGAGGGCTCCTCCCATGCCTCCTGCACACAGCCGTCTGAGCAGG
GCAGGTGCCCAACACCCCCCACCGGAGGACACGCTGCCCCTCAGCGATGCCCCTACC
TTTTGGGGGGCCTCGTCTCAAGCCCCCCCTTGGAGGCTGAAATCACCCCAGGCACTG
TGAGGGCTTCTCCAGGGGGCACCCTTTGAGCTGTGGGTCTGATCACCCCAAGTCCCG
CCCGGAGGAGAGGCACAGCCAGGGCGTGTGGTTTAATGTTTGCCCCCTTCGGGGCTG
GAGGTCTCAGTGTTTCTAGATTCCAGACCCTGCTGCCAGAGAGACCTGCTGCCGGAG
AGAAGGGGAGGAGGACTCCAGCTGGGCTCGGTCCCCCACAGTCAGGGACCCCCATA
AAGGACACCCCCTTCTCTCTAGAAAGAGCTGGGCTCTCAGCTATTTCTAGTTGCTTCC
CAGAAGCCGAGGAGCAGAAGGAGCTGTGAGAGCTTTGCAGAAACGCCCTTGTCCCC
GCCCTCCTGAGCTATGAATGCCGTACAGAGCAGAGGCTGGGGCATTGGCAAGATCA
CAGGTTGATGCTGCACAGCCCCATTGACACAAACCCTCAAAGCAGACGTGAGAGGG
ACGGTTCACAAAGCTCGGACCTGCCGTGGAGGGTGCCCGGCAGACGTGGCGTGAGA
GGGACGGCTCACGAGGCTTGGACCTGCTGTGGAGGGTGCCCAGCAGACGTGGTGTG
AGAGGAACGGCTCACGAGACTTGGACCTGGTGGAGGGTGCCCAGCAGACGTGGTGT
GAGAGGGACGGCTCACAGGGCTTGGACCTGCCATGGAGGGTGCCCGGCAGACGTGG
TGTGAGAGGGATGGTTCACAGGGCTTGGACCTGCCATGGAGGGTGCCCGGCAGATG
TGGTGGGAGAGAGATGGCTCATGAGGCTTGGACCTGCCGTGGAGGGTGCCCAGCAG
ACGTGGTATGAGAGGGATGGCTCACGAGGCTTGGACCTGGTGGAGGGTGCCCGGCA
GACGTGTGAGAGGGACGGTTCACAAGGCTTGGACCTGCCATGGAGGGTGCCCAGCA
GACGTGGTGTGAGAGGGACAGCTCACGAGGCTTGGACCTGCTGTGGAGGGTGCCCA
GCAGGGGGCTGAGCTCTGAGGGGTGGGTGCTCAGTGCACGGGTGCCCCCAGTGTCCT
CTGATCCTGTCCGGTGCCTCCCCCAACCCCCACACCCATGCAGAACTCCCAGGTCAC
ATGCACGTATGTCCAGGGCATGGGGGTGGCGTGAAGAGGCCTGGTCAGGGCCTTTA
GGGGCTGCAGGACGGAATGGCCGCCTGGGGAGCCTGTGTGGCTGTGCCGGGCAGCC
ATCCTGCATTCCCACCCAGCGCGCAGTCTCCACCTCGGCCCCAGCAAAGCGCTAAGC
AGCCGGAGAGACAGCCAGGGCGGCTTCCTGAAGGATGTGGGATGGTGGACTCCGGG
GTCGAGGGAATACGCAGGTTCCTGTCCCTCCGGGAGACCTAGAGAAGCTGCACACC
CAGGAGCTTTCCATGACCCGGGAGCATGAGTGAATGGGGGTTCCAGTTTGCTGAACT
TTGCCGTCTTGTAAGGGTGGGGGCTGACGGCCGACCCTGGGAGGAGGTGACATCGC
CGGGGGAGGTTGTGGGCAACGGTGGAGGAGGAGAGACGGGAGGGGACCATTTGGG
ATGGAGGGGCCTCTTCAGAGTTTTAAAAGGCGTTTGTGGGGTGGAGTTGAGTGTGCT
CTGGGCTTGGACACTTGCCGTGGTGCCCCTGGCTGGCCGAGGAGACTGGCTCTGGCC
AGGGGCCCCGTCCTGAGAGGTCCTCAGCGTCTGACTCTCGGCCAGGCGCCAGCAAG
GAGGGGCCGGTCCCCGGGGCTACCAGGCAGGCACGTGCACATCGCCATCGCCACAC
GCCAACTCCGCCTGGGTTTTACAAAGTCGTTGCCTTAATGCATGTGGACAGGAACTC
CCTGAGGTCGCCCCATGCCCCCTGGCTGTGCCAGGTACGGACGCCCTGGACCCTGCG
AACAGGTGGGGGGGGCGAGGGGCCCAAGGGACGGGCTCCAGAGACACGCGCAGGG
CAGGAGGGGTCTCACGGAGGGGTCTCGCACTGAGGCGCCCAGAGCTGGTGGTCCCG
CTGGACGCCATCCCTCTGCCCGGGATCCACACGGCCCACGTGTGCCCGCCATGCCCG
CGCCCCACGCCATTGCAGTCTGCCATCCTCTGGCCGTGACGGTGGCTGCAGCTTCCC
CATTTGCGCCGTTGCCTCTGGCTGTCTGCACTTTTGTTCATGCTCCAAAGAACATTTC
ATAATGCCTTCAGTACCGACGTACACTTCTGACCATTTTGTATGTGTCCTTGTGCCGT
AGTGACCAGGCCTTTTTTTGGTGGATGTGTTACCCCGCACACTTCAATCTCAACTTTG
TGCACCGTCCATTTTCTAGGGATAGACGCCCAGGGAATGAACTCTAGTTTTCTAACA
GATTAGCTGAGATATTAACTTACTCACACGGACAGGTTGATGCCAGAGCCGTAAGAA
TGCGCCAGTGCGGGTTTGCGGGGGACTTCGGGTGTGGGGTCCTGCGGCCGCGATGGC
CGTGGAAGGTTCTGGGGATCCCTGCTGCCACGGGGACGAGTTCGGACGCCAGGTGG
ACCTGTGCACTCAGTAAAACGCAGTGATTCAACCTGGA
(Source: NCBI Reference Sequence: XM_017027844.2)

In various embodiments, a KCNQ2 miRNA transcript comprises the sequence provided as SEQ ID NO: 3040.

(SEQ ID NO: 3040)
AGCGTCTCCGCGCGCGGCCCAAGCCCGGCAGGAGTGCGGAACCGCCGCCTCGGCCA
TGCGGCTCCCGGCCGGGGGGCCTGGGCTGGGGCCCGCGCCGCCCCCCGCGCTCCGCC
CCCGCTGAGCCTGAGCCCGACCCGGGGCGCCTCCCGCCAGGCACCATGGTGCAGAA
GTCGCGCAACGGCGGCGTATACCCCGGCCCGAGCGGGGAGAAGAAGCTGAAGGTGG
GCTTCGTGGGGCTGGACCCCGGCGCGCCCGACTCCACCCGGGACGGGGCGCTGCTG
ATCGCCGGCTCCGAGGCCCCCAAGCGCGGCAGCATCCTCAGCAAACCTCGCGCGGG
CGGCGCGGGCGCCGGGAAGCCCCCCAAGCGCAACGCCTTCTACCGCAAGCTGCAGA
ATTTCCTCTACAACGTGCTGGAGCGGCCGCGCGGCTGGGCGTTCATCTACCACGCCT
ACGTGTTCCTCCTGGTTTTCTCCTGCCTCGTGCTGTCTGTGTTTTCCACCATCAAGGA
GTATGAGAAGAGCTCGGAGGGGGCCCTCTACATCCTGGAAATCGTGACTATCGTGGT
GTTTGGCGTGGAGTACTTCGTGCGGATCTGGGCCGCAGGCTGCTGCTGCCGGTACCG
TGGCTGGAGGGGGCGGCTCAAGTTTGCCCGGAAACCGTTCTGTGTGATTGACATCAT
GGTGCTCATCGCCTCCATTGCGGTGCTGGCCGCCGGCTCCCAGGGCAACGTCTTTGC
CACATCTGCGCTCCGGAGCCTGCGCTTCCTGCAGATTCTGCGGATGATCCGCATGGA
CCGGCGGGGAGGCACCTGGAAGCTGCTGGGCTCTGTGGTCTATGCCCACAGCAAGG
AGCTGGTCACTGCCTGGTACATCGGCTTCCTTTGTCTCATCCTGGCCTCGTTCCTGGT
GTACTTGGCAGAGAAGGGGGAGAACGACCACTTTGACACCTACGCGGATGCACTCT
GGTGGGGCCTGATCACGCTGACCACCATTGGCTACGGGGACAAGTACCCCCAGACC
TGGAACGGCAGGCTCCTTGCGGCAACCTTCACCCTCATCGGTGTCTCCTTCTTCGCGC
TGCCTGCAGGCATCTTGGGGTCTGGGTTTGCCCTGAAGGTTCAGGAGCAGCACAGGC
AGAAGCACTTTGAGAAGAGGCGGAACCCGGCAGCAGGCCTGATCCAGTCGGCCTGG
AGATTCTACGCCACCAACCTCTCGCGCACAGACCTGCACTCCACGTGGCAGTACTAC
GAGCGAACGGTCACCGTGCCCATGTACAGTTCGCAAACTCAAACCTACGGGGCCTCC
AGACTTATCCCCCCGCTGAACCAGCTGGAGCTGCTGAGGAACCTCAAGAGTAAATCT
GGACTCGCTTTCAGGAAGGACCCCCCGCCGGAGCCGTCTCCAAGCCAGAAGGTCAG
TTTGAAAGATCGTGTCTTCTCCAGCCCCCGAGGCGTGGCTGCCAAGGGGAAGGGGTC
CCCGCAGGCCCAGACTGTGAGGCGGTCACCCAGCGCCGACCAGAGCCTCGAGGACA
GCCCCAGCAAGGTGCCCAAGAGCTGGAGCTTCGGGGACCGCAGCCGGGCACGCCAG
GCTTTCCGCATCAAGGGTGCCGCGTCACGGCAGAACTCAGAAGCAAGCCTCCCCGG
AGAGGACATTGTGGATGACAAGAGCTGCCCCTGCGAGTTTGTGACCGAGGACCTGA
CCCCGGGCCTCAAAGTCAGCATCAGAGCCGTGTGTGTCATGCGGTTCCTGGTGTCCA
AGCGGAAGTTCAAGGAGAGCCTGCGGCCCTACGACGTGATGGACGTCATCGAGCAG
TACTCAGCCGGCCACCTGGACATGCTGTCCCGAATTAAGAGCCTGCAGTCCAGGATA
GATATGATTGTGGGTCCCCCGCCCCCTTCAACTCCCCGGCACAAGAAGTACCCCACC
AAAGGACCCACGGCCCCTCCGAGAGAGTCACCCCAGTACTCACCTAGAGTGGACCA
GATCGTGGGGCGGGGCCCAGCGATCACGGACAAGGACCGCACCAAGGGCCCGGCCG
AGGCGGAGCTGCCCGAGGACCCCAGCATGATGGGACGGCTCGGGAAGGTGGAGAA
GCAGGTCTTGTCCATGGAGAAGAAGCTGGACTTCCTGGTGAATATCTACATGCAGCG
GATGGGCATCCCCCCGACAGAGACCGAGGCCTACTTTGGGGCCAAAGAGCCGGAGC
CGGCGCCGCCGTACCACAGCCCGGAAGACAGCCGGGAGCATGTCGACAGGCACGGC
TGCATTGTCAAGATCGTGCGCTCCAGCAGCTCCACGGGCCAGAAGAACTTCTCGGCG
CCCCCGGCCGCGCCCCCTGTCCAGTGTCCGCCCTCCACCTCCTGGCAGCCACAGAGC
CACCCGCGCCAGGGCCACGGCACCTCCCCCGTGGGGGACCACGGCTCCCTGGTGCG
CATCCCGCCGCCGCCTGCCCACGAGCGGTCGCTGTCCGCCTACGGCGGGGGCAACCG
CGCCAGCATGGAGTTCCTGCGGCAGGAGGACACCCCGGGCTGCAGGCCCCCCGAGG
GGAACCTGCGGGACAGCGACACGTCCATCTCCATCCCGTCCGTGGACCACGAGGAG
CTGGAGCGTTCCTTCAGCGGCTTCAGCATCTCCCAGTCCAAGGAGAACCTGGATGCT
CTCAACAGCTGCTACGCGGCCGTGGCGCCTTGTGCCAAAGTCAGGCCCTACATTGCG
GAGGGAGAGTCAGACACCGACTCCGACCTCTGTACCCCGTGCGGGCCCCCGCCACG
CTCGGCCACCGGCGAGGGTCCCTTTGGTGACGTGGGCTGGGCCGGGCCCAGGAAGT
GAGGCGGCGCTGGGCCAGTGGACCCGCCCGCGGCCCTCCTCAGCACGGTGCCTCCG
AGGTTTTGAGGCGGGAACCCTCTGGGGCCCTTTTCTTACAGTAACTGAGTGTGGCGG
GAAGGGTGGGCCCTGGAGGGGCCCATGTGGGCTGAAGGATGGGGGCTCCTGGCAGT
GACCTTTTACAAAAGTTATTTTCCAACAGGGGCTGGAGGGCTGGGCAGGGCCCTGTG
GCTCCAGGAGCAGCGTGCAGGAGCAAGGCTGCCCTGTCCACTCTGCTCAGGGCCGC
GGCCGACATCAGCCCGGTGTGAGGAGGGGGGGGAGTGATGACGGGGTGTTGCCAGC
GTGGCAACAGGCGGGGGGTTGTCTCAGCCGAGCCCAGGGGAGGCACAAAGGGCAG
GCCTGTTCCCTGAGGACCTGCGCAAAGGGCGGGCCTGTTTGGTGAGGACCTGCGGCC
TTGGGTCCCGGTGGGGTTTCCGGGCAGCTACAGGCGGGTGTGGCCGGCCGCTGTGCG
TGGCCTCTGCCTTCACACCTGACCTGCCCGGCGGGCTTTCCTGTTCCCCACCTCAGGG
GCGCCCAAATACAGAGCTATTGGTTGGCGTCTTCTCCCTGTACCTTCTGGGATCTGAG
GGCTCTTTCCATGGAAGCCAGCCCCGAGGTGGAGACCTTCGCCTGCAGCCGAGGAG
CGGGTGGGGCCTGGGAACCAAACTGGAGCCAGAGTGGACGTCCAGCCCTCTGGTCT
TGGCCTCCAGAGGGAGGGCCTGGCTCACGGTGGGGCCAGGGAGCCGGCTCCAAAGG
GTCTTCAAAAAGGGGGTCCTTGGGGGCTCCAGCTGCCTCGCCCTGGCCTTTCTGTGG
GTGCGTGAGAGCCAGCAGCACCCCAGCCTTGGAGACCGGGGGGGCAGGACCCCAAG
TCCTCCCCTCTCTCCTGACTGCCCTGGCCGGGTGCCGGCACTGCGAGACCCACCTGG
TGAGCAGGCCTCACAGTTCTTAGCCAGGGCCCCACCTCGCCTGTGTCCCACCAGTGC
CCCGACAGACCTGGGGCAGGGCTGGGCCATGATGCAGCGGGCCAGGATAGCCTCCA
CCGTCAGCACAGGGCCGCCCTCCCCGCCTTTCCGGAGGAAACCACTCCCACCTCAGC
CCAGCTGTGCGCCCTCCCTAGCTCTCCTGCCCCCTGGAGCTGATGGCCCCTTCTCCAC
TGACCGATTCCTTAGCGGGGCCTCTTGGGGTCTCGGGCCTCGGGTGCACCGTCCCAT
GCCCGTCCTGTTGTGGGCACCGTGGCCCTTGGGGCAGGCGGCTCTAATGCGGGAGCG
AGTCCCTAGCTCCAGACTTAAGAACCAGACCCCGGGAGCATCTGGCATTTGGCGTGA
CGGCGTCGCAGGCGGGCCTGGGCTCCCTGGAGAGTGGCCTCCCTGGGAGTGAGCAG
GGCTGGGGTCGTGGGCGCAAATACTCCTGCAGAGCAAGTGCAGGGGAGTCCTGGGC
CCGTTTCTCCTCCACCTGCGTTTTCAGTGCACTTGGCTTGGCTGGGAGGTCCTGAGGC
CCTGAGGCCAGCAGGGGAACCAGTCCTGAGGGAGAGGACTTTGAAAGCAGCATTTG
AGGGTCGTACGCCCCTGGCTGGTGGGGGTCCTGGCGCTCAGGGTGTTCGGGGAGCCA
TGTCTGGCGTCCATTGTGGGGAGCTGCTGCCCTGGCCTCTCTGCCTACCCCCAGCCCG
GCCAGGGCACTCCCAGGCCCTGTCGCCATTGAGGTGCCTCCGCTGGGCTGTCTCCTC
ACCCCTCCCTGTGCTGGAGCCTGTCCCAAAAAGGTGCCAACTGGGAGGCCTCGGAA
GCCACTGTCCAGGCTCCCACTGCCTGTCTGCTCTGTTCCCAAAGGCAGCGTGTGTGG
CCTCGGGCCCTGCGGTGGCATGAAGCATCCCTTCTGGTGTGGGCATCGCTACGTGTT
TTGGGGGCAGCGTTTCACGGCGGTGCCCTTGCTGTCTCCCTTGGGCTGGCTCGAGCCT
GGGGTCCATGTCCCTTTGCCGTCCCGTCATGGGGCAGGGAATCCATAGCGGGGCCCA
CAGGCAGGGGTATGAGTGCGTCCCACCCAACGCAGCACCAGCCCCGGCCACCGCTC
CCCGTGTCCCCAGTTCCGTCTCAGCTACCTGGACTCCAGGACCCTGGAGAAGGGAGA
CCTGGCAGTGGAGGGAGGCTGTGCTGTGTGTCCCCCTGCAGGTGTGACCCCGCCTGC
TCTTTCCTCCCCCGCCAGGTGTGGCCCCGCCTGCTCTTTCCTCCCCCACCAGTATGGC
CCCACCTGCTCTTTCCTCCCCCCCCAAGGTGTGGCCCCACCTGTTCTTTCCTCCCCTG
CCGAGGTGTGACCCCACCTGCTCTTTCCTCCCTCCCAGTATGGCCCCACCTGCTCTTT
CCTCCCCCGAGGTGAGGCCCCGCCTGCTCTTTCCTCCCATGGGGCCGCTGAGGCATG
AGCACCTGGGCACAGGTTGGGGCTCTGCAGGATGAGGAAGACAGGCCAATCCCTTC
CCTCCCAGAAGCTGGCCGCCCAGCAGGAGGGACTGAGGCCAGACTCATGTCCAGCA
AGGAACGTGTGGTGTGTCCCCTGGGAAGTCTCTGGGCCCTGGGAAGAGGGAAGGTG
CACGTCCTGGGATGGTTGCGGGGCCCTGTTTTGGGAGACAAAGGGGTAGAGGGTCT
GTCTTGGGCCCCCCCAGACTCTAGCCTGAGCAGTGCAGCCACCTACTGCCCCACCTC
AGAGAAGTGCAGCGGGAAGGAGGCTGGAGGTGGTGCGGCGCTGCCTCGGGTGTCTG
CGTGAATGAGCGTGGCCAAGGACCAGTGCCACCTCATGGCAAAGAGCTCCCGCAGT
GTTTGTTAGAGTGCACATCCCTACGTGCCCACTGGCACACACACGTGCTCACATACA
TGTCCGCATACAGGCGTACACATGCACGCTTGCACACATGCACACAGACCACATAGC
ACACATGTGCACTGACCACACCTGTATAGACCATGCACAGTACACATACGTGCATAC
ACATGCCTGCATACAGGCATACACATGCACGCTTACATGTACACGTGCACAGATCAC
ACACATGCACACACGTGTAGCTCACACACAGTATACACATACACAAGTGCACAGAC
CACACACAGCACTAACACATGCACACACAAAGTGCATAGGCCACACAGCACATGCA
CACAGGTGCACAGACCACACAGCACACACAAGTGCACAGAGCACACTGCACACATG
CACACACACACGCGTGCATGCACACTCCTCGCACTTCCAGCCTTGGAGCCCTTCTGT
CTCTGGTCTTTCTCTTTGACCCTGCTGAGTGTAAGCTGCCTGGGGAGGGGCTACAAG
GAGTAATTGTGGCTTTAGGGGTCGTGGTGATGCTGGAATGTCAAGCGCCGTCGTGGG
GTATCCGACTGTCCGGGCTCCTGGTCCGCAGTGGCAGAGCGCCAGGCAGAGCCCAA
TCAGGGTCTCGTGCTGCCCTTCCCTCCCACAGCCTGGCAGCCATCCAGAGGAGGGGC
TCTACCAGATGCCAAGGTGCCCCGGTGTCTGTATGGGTGTCCGGTTGGGTCCTGTGTT
TGGTCTGCCCTGGAGGTGCTGGGCCCTCCTGGGATGGGTGGCTCAGCCTCGAATCCC
AGGCCCCAGCCCAGGCAGGTGCTGCTGCCTGTTGTGGTTTCCTGGCCCAGCTTCTCCT
TCTCCCTCTGCATAAAATCACAGTCCGTGAGTCTTCCAGCTGCCACCACGGCTGGGA
CACGCTGGGGGAGGGCTCCTCCCATGCCTCCTGCACACAGCCGTCTGAGCAGGGCA
GGTGCCCAACACCCCCCACCGGAGGACACGCTGCCCCTCAGCGATGCCCCTACCTTT
TGGGGGGCCTCGTCTCAAGCCCCCCCTTGGAGGCTGAAATCACCCCAGGCACTGTGA
GGGCTTCTCCAGGGGGCACCCTTTGAGCTGTGGGTCTGATCACCCCAAGTCCCGCCC
GGAGGAGAGGCACAGCCAGGGCGTGTGGTTTAATGTTTGCCCCCTTCGGGGCTGGA
GGTCTCAGTGTTTCTAGATTCCAGACCCTGCTGCCAGAGAGACCTGCTGCCGGAGAG
AAGGGGAGGAGGACTCCAGCTGGGCTCGGTCCCCCACAGTCAGGGACCCCCATAAA
GGACACCCCCTTCTCTCTAGAAAGAGCTGGGCTCTCAGCTATTTCTAGTTGCTTCCCA
GAAGCCGAGGAGCAGAAGGAGCTGTGAGAGCTTTGCAGAAACGCCCTTGTCCCCGC
CCTCCTGAGCTATGAATGCCGTACAGAGCAGAGGCTGGGGCATTGGCAAGATCACA
GGTTGATGCTGCACAGCCCCATTGACACAAACCCTCAAAGCAGACGTGAGAGGGAC
GGTTCACAAAGCTCGGACCTGCCGTGGAGGGTGCCCGGCAGACGTGGCGTGAGAGG
GACGGCTCACGAGGCTTGGACCTGCTGTGGAGGGTGCCCAGCAGACGTGGTGTGAG
AGGAACGGCTCACGAGACTTGGACCTGGTGGAGGGTGCCCAGCAGACGTGGTGTGA
GAGGGACGGCTCACAGGGCTTGGACCTGCCATGGAGGGTGCCCGGCAGACGTGGTG
TGAGAGGGATGGTTCACAGGGCTTGGACCTGCCATGGAGGGTGCCCGGCAGATGTG
GTGGGAGAGAGATGGCTCATGAGGCTTGGACCTGCCGTGGAGGGTGCCCAGCAGAC
GTGGTATGAGAGGGATGGCTCACGAGGCTTGGACCTGGTGGAGGGTGCCCGGCAGA
CGTGTGAGAGGGACGGTTCACAAGGCTTGGACCTGCCATGGAGGGTGCCCAGCAGA
CGTGGTGTGAGAGGGACAGCTCACGAGGCTTGGACCTGCTGTGGAGGGTGCCCAGC
AGGGGGCTGAGCTCTGAGGGGTGGGTGCTCAGTGCACGGGTGCCCCCAGTGTCCTCT
GATCCTGTCCGGTGCCTCCCCCAACCCCCACACCCATGCAGAACTCCCAGGTCACAT
GCACGTATGTCCAGGGCATGGGGGTGGCGTGAAGAGGCCTGGTCAGGGCCTTTAGG
GGCTGCAGGACGGAATGGCCGCCTGGGGAGCCTGTGTGGCTGTGCCGGGCAGCCAT
CCTGCATTCCCACCCAGCGCGCAGTCTCCACCTCGGCCCCAGCAAAGCGCTAAGCAG
CCGGAGAGACAGCCAGGGCGGCTTCCTGAAGGATGTGGGATGGTGGACTCCGGGGT
CGAGGGAATACGCAGGTTCCTGTCCCTCCGGGAGACCTAGAGAAGCTGCACACCCA
GGAGCTTTCCATGACCCGGGAGCATGAGTGAATGGGGGTTCCAGTTTGCTGAACTTT
GCCGTCTTGTAAGGGTGGGGGCTGACGGCCGACCCTGGGAGGAGGTGACATCGCCG
GGGGAGGTTGTGGGCAACGGTGGAGGAGGAGAGACGGGAGGGGACCATTTGGGAT
GGAGGGGCCTCTTCAGAGTTTTAAAAGGCGTTTGTGGGGTGGAGTTGAGTGTGCTCT
GGGCTTGGACACTTGCCGTGGTGCCCCTGGCTGGCCGAGGAGACTGGCTCTGGCCAG
GGGCCCCGTCCTGAGAGGTCCTCAGCGTCTGACTCTCGGCCAGGCGCCAGCAAGGA
GGGGCCGGTCCCCGGGGCTACCAGGCAGGCACGTGCACATCGCCATCGCCACACGC
CAACTCCGCCTGGGTTTTACAAAGTCGTTGCCTTAATGCATGTGGACAGGAACTCCC
TGAGGTCGCCCCATGCCCCCTGGCTGTGCCAGGTACGGACGCCCTGGACCCTGCGAA
CAGGTGGGGGGGGCGAGGGGCCCAAGGGACGGGCTCCAGAGACACGCGCAGGGCA
GGAGGGGTCTCACGGAGGGGTCTCGCACTGAGGCGCCCAGAGCTGGTGGTCCCGCT
GGACGCCATCCCTCTGCCCGGGATCCACACGGCCCACGTGTGCCCGCCATGCCCGCG
CCCCACGCCATTGCAGTCTGCCATCCTCTGGCCGTGACGGTGGCTGCAGCTTCCCCA
TTTGCGCCGTTGCCTCTGGCTGTCTGCACTTTTGTTCATGCTCCAAAGAACATTTCAT
AATGCCTTCAGTACCGACGTACACTTCTGACCATTTTGTATGTGTCCTTGTGCCGTAG
TGACCAGGCCTTTTTTTGGTGGATGTGTTACCCCGCACACTTCAATCTCAACTTTGTG
CACCGTCCATTTTCTAGGGATAGACGCCCAGGGAATGAACTCTAGTTTTCTAACAGA
TTAGCTGAGATATTAACTTACTCACACGGACAGGTTGATGCCAGAGCCGTAAGAATG
CGCCAGTGCGGGTTTGCGGGGGACTTCGGGTGTGGGGTCCTGCGGCCGCGATGGCCG
TGGAAGGTTCTGGGGATCCCTGCTGCCACGGGGACGAGTTCGGACGCCAGGTGGAC
CTGTGCACTCAGTAAAACGCAGTGATTCAACCTGGA
(Source: NCBI Reference Sequence: XM_017027841.2)

In various embodiments, a KCNQ2 mRNA transcript comprises the sequence provided as SEQ ID NO: 3041.

(SEQ ID NO: 3041)
AGCGTCTCCGCGCGCGGCCCAAGCCCGGCAGGAGTGCGGAACCGCCGCCTCGGCCA
TGCGGCTCCCGGCCGGGGGGCCTGGGCTGGGGCCCGCGCCGCCCCCCGCGCTCCGCC
CCCGCTGAGCCTGAGCCCGACCCGGGGCGCCTCCCGCCAGGCACCATGGTGCAGAA
GTCGCGCAACGGCGGCGTATACCCCGGCCCGAGCGGGGAGAAGAAGCTGAAGGTGG
GCTTCGTGGGGCTGGACCCCGGCGCGCCCGACTCCACCCGGGACGGGGCGCTGCTG
ATCGCCGGCTCCGAGGCCCCCAAGCGCGGCAGCATCCTCAGCAAACCTCGCGCGGG
CGGCGCGGGCGCCGGGAAGCCCCCCAAGCGCAACGCCTTCTACCGCAAGCTGCAGA
ATTTCCTCTACAACGTGCTGGAGCGGCCGCGCGGCTGGGCGTTCATCTACCACGCCT
ACGTGTTCCTCCTGGTTTTCTCCTGCCTCGTGCTGTCTGTGTTTTCCACCATCAAGGA
GTATGAGAAGAGCTCGGAGGGGGCCCTCTACATCCTGGAAATCGTGACTATCGTGGT
GTTTGGCGTGGAGTACTTCGTGCGGATCTGGGCCGCAGGCTGCTGCTGCCGGTACCG
TGGCTGGAGGGGGCGGCTCAAGTTTGCCCGGAAACCGTTCTGTGTGATTGACATCAT
GGTGCTCATCGCCTCCATTGCGGTGCTGGCCGCCGGCTCCCAGGGCAACGTCTTTGC
CACATCTGCGCTCCGGAGCCTGCGCTTCCTGCAGATTCTGCGGATGATCCGCATGGA
CCGGCGGGGAGGCACCTGGAAGCTGCTGGGCTCTGTGGTCTATGCCCACAGCAAGG
AGCTGGTCACTGCCTGGTACATCGGCTTCCTTTGTCTCATCCTGGCCTCGTTCCTGGT
GTACTTGGCAGAGAAGGGGGAGAACGACCACTTTGACACCTACGCGGATGCACTCT
GGTGGGGCCTGATCACGCTGACCACCATTGGCTACGGGGACAAGTACCCCCAGACC
TGGAACGGCAGGCTCCTTGCGGCAACCTTCACCCTCATCGGTGTCTCCTTCTTCGCGC
TGCCTGCAGGCATCTTGGGGTCTGGGTTTGCCCTGAAGGTTCAGGAGCAGCACAGGC
AGAAGCACTTTGAGAAGAGGCGGAACCCGGCAGCAGGCCTGATCCAGTCGGCCTGG
AGATTCTACGCCACCAACCTCTCGCGCACAGACCTGCACTCCACGTGGCAGTACTAC
GAGCGAACGGTCACCGTGCCCATGTACAGTTCGCAAACTCAAACCTACGGGGCCTCC
AGACTTATCCCCCCGCTGAACCAGCTGGAGCTGCTGAGGAACCTCAAGAGTAAATCT
GGACTCGCTTTCAGGAAGGACCCCCCGCCGGAGCCGTCTCCAAGCCAGAAGGTCAG
TTTGAAAGATCGTGTCTTCTCCAGCCCCCGAGGCGTGGCTGCCAAGGGGAAGGGGTC
CCCGCAGGCCCAGACTGTGAGGCGGTCACCCAGCGCCGACCAGAGCCTCGAGGACA
GCCCCAGCAAGGTGCCCAAGAGCTGGAGCTTCGGGGACCGCAGCCGGGCACGCCAG
GCTTTCCGCATCAAGGGTGCCGCGTCACGGCAGAACTCAGAAGAAGCAAGCCTCCC
CGGAGAGGACATTGTGGATGACAAGAGCTGCCCCTGCGAGTTTGTGACCGAGGACC
TGACCCCGGGCCTCAAAGTCAGCATCAGAGCCGTGTGTGTCATGCGGTTCCTGGTGT
CCAAGCGGAAGTTCAAGGAGAGCCTGCGGCCCTACGACGTGATGGACGTCATCGAG
CAGTACTCAGCCGGCCACCTGGACATGCTGTCCCGAATTAAGAGCCTGCAGTCCAGG
CAAGAGCCCCGCCTGCCTGTCCAGCAGGGGACAAGAACGGGAGTGGACCAGATCGT
GGGGGGGGCCCAGCGATCACGGACAAGGACCGCACCAAGGGCCCGGCCGAGGCG
GAGCTGCCCGAGGACCCCAGCATGATGGGACGGCTCGGGAAGGTGGAGAAGCAGGT
CTTGTCCATGGAGAAGAAGCTGGACTTCCTGGTGAATATCTACATGCAGCGGATGGG
CATCCCCCCGACAGAGACCGAGGCCTACTTTGGGGCCAAAGAGCCGGAGCCGGCGC
CGCCGTACCACAGCCCGGAAGACAGCCGGGAGCATGTCGACAGGCACGGCTGCATT
GTCAAGATCGTGCGCTCCAGCAGCTCCACGGGCCAGAAGAACTTCTCGGCGCCCCCG
GCCGCGCCCCCTGTCCAGTGTCCGCCCTCCACCTCCTGGCAGCCACAGAGCCACCCG
CGCCAGGGCCACGGCACCTCCCCCGTGGGGGACCACGGCTCCCTGGTGCGCATCCCG
CCGCCGCCTGCCCACGAGCGGTCGCTGTCCGCCTACGGCGGGGGCAACCGCGCCAG
CATGGAGTTCCTGCGGCAGGAGGACACCCCGGGCTGCAGGCCCCCCGAGGGGAACC
TGCGGGACAGCGACACGTCCATCTCCATCCCGTCCGTGGACCACGAGGAGCTGGAG
CGTTCCTTCAGCGGCTTCAGCATCTCCCAGTCCAAGGAGAACCTGGATGCTCTCAAC
AGCTGCTACGCGGCCGTGGCGCCTTGTGCCAAAGTCAGGCCCTACATTGCGGAGGGA
GAGTCAGACACCGACTCCGACCTCTGTACCCCGTGCGGGCCCCCGCCACGCTCGGCC
ACCGGCGAGGGTCCCTTTGGTGACGTGGGCTGGGCCGGGCCCAGGAAGTGAGGCGG
CGCTGGGCCAGTGGACCCGCCCGCGGCCCTCCTCAGCACGGTGCCTCCGAGGTTTTG
AGGCGGGAACCCTCTGGGGCCCTTTTCTTACAGTAACTGAGTGTGGCGGGAAGGGTG
GGCCCTGGAGGGGCCCATGTGGGCTGAAGGATGGGGGCTCCTGGCAGTGACCTTTTA
CAAAAGTTATTTTCCAACAGGGGCTGGAGGGCTGGGCAGGGCCCTGTGGCTCCAGG
AGCAGCGTGCAGGAGCAAGGCTGCCCTGTCCACTCTGCTCAGGGCCGCGGCCGACA
TCAGCCCGGTGTGAGGAGGGGGGGGAGTGATGACGGGGTGTTGCCAGCGTGGCAAC
AGGCGGGGGGTTGTCTCAGCCGAGCCCAGGGGAGGCACAAAGGGCAGGCCTGTTCC
CTGAGGACCTGCGCAAAGGGCGGGCCTGTTTGGTGAGGACCTGCGGCCTTGGGTCCC
GGTGGGGTTTCCGGGCAGCTACAGGCGGGTGTGGCCGGCCGCTGTGCGTGGCCTCTG
CCTTCACACCTGACCTGCCCGGCGGGCTTTCCTGTTCCCCACCTCAGGGGCGCCCAA
ATACAGAGCTATTGGTTGGCGTCTTCTCCCTGTACCTTCTGGGATCTGAGGGCTCTTT
CCATGGAAGCCAGCCCCGAGGTGGAGACCTTCGCCTGCAGCCGAGGAGCGGGTGGG
GCCTGGGAACCAAACTGGAGCCAGAGTGGACGTCCAGCCCTCTGGTCTTGGCCTCCA
GAGGGAGGGCCTGGCTCACGGTGGGGCCAGGGAGCCGGCTCCAAAGGGTCTTCAAA
AAGGGGGTCCTTGGGGGCTCCAGCTGCCTCGCCCTGGCCTTTCTGTGGGTGCGTGAG
AGCCAGCAGCACCCCAGCCTTGGAGACCGGGGGGGCAGGACCCCAAGTCCTCCCCT
CTCTCCTGACTGCCCTGGCCGGGTGCCGGCACTGCGAGACCCACCTGGTGAGCAGGC
CTCACAGTTCTTAGCCAGGGCCCCACCTCGCCTGTGTCCCACCAGTGCCCCGACAGA
CCTGGGGCAGGGCTGGGCCATGATGCAGCGGGCCAGGATAGCCTCCACCGTCAGCA
CAGGGCCGCCCTCCCCGCCTTTCCGGAGGAAACCACTCCCACCTCAGCCCAGCTGTG
CGCCCTCCCTAGCTCTCCTGCCCCCTGGAGCTGATGGCCCCTTCTCCACTGACCGATT
CCTTAGCGGGGCCTCTTGGGGTCTCGGGCCTCGGGTGCACCGTCCCATGCCCGTCCT
GTTGTGGGCACCGTGGCCCTTGGGGCAGGCGGCTCTAATGCGGGAGCGAGTCCCTAG
CTCCAGACTTAAGAACCAGACCCCGGGAGCATCTGGCATTTGGCGTGACGGCGTCGC
AGGCGGGCCTGGGCTCCCTGGAGAGTGGCCTCCCTGGGAGTGAGCAGGGCTGGGGT
CGTGGGCGCAAATACTCCTGCAGAGCAAGTGCAGGGGAGTCCTGGGCCCGTTTCTCC
TCCACCTGCGTTTTCAGTGCACTTGGCTTGGCTGGGAGGTCCTGAGGCCCTGAGGCC
AGCAGGGGAACCAGTCCTGAGGGAGAGGACTTTGAAAGCAGCATTTGAGGGTCGTA
CGCCCCTGGCTGGTGGGGGTCCTGGCGCTCAGGGTGTTCGGGGAGCCATGTCTGGCG
TCCATTGTGGGGAGCTGCTGCCCTGGCCTCTCTGCCTACCCCCAGCCCGGCCAGGGC
ACTCCCAGGCCCTGTCGCCATTGAGGTGCCTCCGCTGGGCTGTCTCCTCACCCCTCCC
TGTGCTGGAGCCTGTCCCAAAAAGGTGCCAACTGGGAGGCCTCGGAAGCCACTGTCC
AGGCTCCCACTGCCTGTCTGCTCTGTTCCCAAAGGCAGCGTGTGTGGCCTCGGGCCC
TGCGGTGGCATGAAGCATCCCTTCTGGTGTGGGCATCGCTACGTGTTTTGGGGGCAG
CGTTTCACGGCGGTGCCCTTGCTGTCTCCCTTGGGCTGGCTCGAGCCTGGGGTCCATG
TCCCTTTGCCGTCCCGTCATGGGGCAGGGAATCCATAGCGGGGCCCACAGGCAGGG
GTATGAGTGCGTCCCACCCAACGCAGCACCAGCCCCGGCCACCGCTCCCCGTGTCCC
CAGTTCCGTCTCAGCTACCTGGACTCCAGGACCCTGGAGAAGGGAGACCTGGCAGTG
GAGGGAGGCTGTGCTGTGTGTCCCCCTGCAGGTGTGACCCCGCCTGCTCTTTCCTCCC
CCGCCAGGTGTGGCCCCGCCTGCTCTTTCCTCCCCCACCAGTATGGCCCCACCTGCTC
TTTCCTCCCCCCCCAAGGTGTGGCCCCACCTGTTCTTTCCTCCCCTGCCGAGGTGTGA
CCCCACCTGCTCTTTCCTCCCTCCCAGTATGGCCCCACCTGCTCTTTCCTCCCCCGAG
GTGAGGCCCCGCCTGCTCTTTCCTCCCATGGGGCCGCTGAGGCATGAGCACCTGGGC
ACAGGTTGGGGCTCTGCAGGATGAGGAAGACAGGCCAATCCCTTCCCTCCCAGAAG
CTGGCCGCCCAGCAGGAGGGACTGAGGCCAGACTCATGTCCAGCAAGGAACGTGTG
GTGTGTCCCCTGGGAAGTCTCTGGGCCCTGGGAAGAGGGAAGGTGCACGTCCTGGG
ATGGTTGCGGGGCCCTGTTTTGGGAGACAAAGGGGTAGAGGGTCTGTCTTGGGCCCC
CCCAGACTCTAGCCTGAGCAGTGCAGCCACCTACTGCCCCACCTCAGAGAAGTGCAG
CGGGAAGGAGGCTGGAGGTGGTGCGGCGCTGCCTCGGGTGTCTGCGTGAATGAGCG
TGGCCAAGGACCAGTGCCACCTCATGGCAAAGAGCTCCCGCAGTGTTTGTTAGAGTG
CACATCCCTACGTGCCCACTGGCACACACACGTGCTCACATACATGTCCGCATACAG
GCGTACACATGCACGCTTGCACACATGCACACAGACCACATAGCACACATGTGCACT
GACCACACCTGTATAGACCATGCACAGTACACATACGTGCATACACATGCCTGCATA
CAGGCATACACATGCACGCTTACATGTACACGTGCACAGATCACACACATGCACAC
ACGTGTAGCTCACACACAGTATACACATACACAAGTGCACAGACCACACACAGCAC
TAACACATGCACACACAAAGTGCATAGGCCACACAGCACATGCACACAGGTGCACA
GACCACACAGCACACACAAGTGCACAGAGCACACTGCACACATGCACACACACACG
CGTGCATGCACACTCCTCGCACTTCCAGCCTTGGAGCCCTTCTGTCTCTGGTCTTTCT
CTTTGACCCTGCTGAGTGTAAGCTGCCTGGGGAGGGGCTACAAGGAGTAATTGTGGC
TTTAGGGGTCGTGGTGATGCTGGAATGTCAAGCGCCGTCGTGGGGTATCCGACTGTC
CGGGCTCCTGGTCCGCAGTGGCAGAGCGCCAGGCAGAGCCCAATCAGGGTCTCGTG
CTGCCCTTCCCTCCCACAGCCTGGCAGCCATCCAGAGGAGGGGCTCTACCAGATGCC
AAGGTGCCCCGGTGTCTGTATGGGTGTCCGGTTGGGTCCTGTGTTTGGTCTGCCCTGG
AGGTGCTGGGCCCTCCTGGGATGGGTGGCTCAGCCTCGAATCCCAGGCCCCAGCCCA
GGCAGGTGCTGCTGCCTGTTGTGGTTTCCTGGCCCAGCTTCTCCTTCTCCCTCTGCAT
AAAATCACAGTCCGTGAGTCTTCCAGCTGCCACCACGGCTGGGACACGCTGGGGGA
GGGCTCCTCCCATGCCTCCTGCACACAGCCGTCTGAGCAGGGCAGGTGCCCAACACC
CCCCACCGGAGGACACGCTGCCCCTCAGCGATGCCCCTACCTTTTGGGGGGCCTCGT
CTCAAGCCCCCCCTTGGAGGCTGAAATCACCCCAGGCACTGTGAGGGCTTCTCCAGG
GGGCACCCTTTGAGCTGTGGGTCTGATCACCCCAAGTCCCGCCCGGAGGAGAGGCA
CAGCCAGGGCGTGTGGTTTAATGTTTGCCCCCTTCGGGGCTGGAGGTCTCAGTGTTTC
TAGATTCCAGACCCTGCTGCCAGAGAGACCTGCTGCCGGAGAGAAGGGGAGGAGGA
CTCCAGCTGGGCTCGGTCCCCCACAGTCAGGGACCCCCATAAAGGACACCCCCTTCT
CTCTAGAAAGAGCTGGGCTCTCAGCTATTTCTAGTTGCTTCCCAGAAGCCGAGGAGC
AGAAGGAGCTGTGAGAGCTTTGCAGAAACGCCCTTGTCCCCGCCCTCCTGAGCTATG
AATGCCGTACAGAGCAGAGGCTGGGGCATTGGCAAGATCACAGGTTGATGCTGCAC
AGCCCCATTGACACAAACCCTCAAAGCAGACGTGAGAGGGACGGTTCACAAAGCTC
GGACCTGCCGTGGAGGGTGCCCGGCAGACGTGGCGTGAGAGGGACGGCTCACGAGG
CTTGGACCTGCTGTGGAGGGTGCCCAGCAGACGTGGTGTGAGAGGAACGGCTCACG
AGACTTGGACCTGGTGGAGGGTGCCCAGCAGACGTGGTGTGAGAGGGACGGCTCAC
AGGGCTTGGACCTGCCATGGAGGGTGCCCGGCAGACGTGGTGTGAGAGGGATGGTT
CACAGGGCTTGGACCTGCCATGGAGGGTGCCCGGCAGATGTGGTGGGAGAGAGATG
GCTCATGAGGCTTGGACCTGCCGTGGAGGGTGCCCAGCAGACGTGGTATGAGAGGG
ATGGCTCACGAGGCTTGGACCTGGTGGAGGGTGCCCGGCAGACGTGTGAGAGGGAC
GGTTCACAAGGCTTGGACCTGCCATGGAGGGTGCCCAGCAGACGTGGTGTGAGAGG
GACAGCTCACGAGGCTTGGACCTGCTGTGGAGGGTGCCCAGCAGGGGGCTGAGCTC
TGAGGGGTGGGTGCTCAGTGCACGGGTGCCCCCAGTGTCCTCTGATCCTGTCCGGTG
CCTCCCCCAACCCCCACACCCATGCAGAACTCCCAGGTCACATGCACGTATGTCCAG
GGCATGGGGGTGGCGTGAAGAGGCCTGGTCAGGGCCTTTAGGGGCTGCAGGACGGA
ATGGCCGCCTGGGGAGCCTGTGTGGCTGTGCCGGGCAGCCATCCTGCATTCCCACCC
AGCGCGCAGTCTCCACCTCGGCCCCAGCAAAGCGCTAAGCAGCCGGAGAGACAGCC
AGGGCGGCTTCCTGAAGGATGTGGGATGGTGGACTCCGGGGTCGAGGGAATACGCA
GGTTCCTGTCCCTCCGGGAGACCTAGAGAAGCTGCACACCCAGGAGCTTTCCATGAC
CCGGGAGCATGAGTGAATGGGGGTTCCAGTTTGCTGAACTTTGCCGTCTTGTAAGGG
TGGGGGCTGACGGCCGACCCTGGGAGGAGGTGACATCGCCGGGGGAGGTTGTGGGC
AACGGTGGAGGAGGAGAGACGGGAGGGGACCATTTGGGATGGAGGGGCCTCTTCAG
AGTTTTAAAAGGCGTTTGTGGGGTGGAGTTGAGTGTGCTCTGGGCTTGGACACTTGC
CGTGGTGCCCCTGGCTGGCCGAGGAGACTGGCTCTGGCCAGGGGCCCCGTCCTGAG
AGGTCCTCAGCGTCTGACTCTCGGCCAGGCGCCAGCAAGGAGGGGCCGGTCCCCGG
GGCTACCAGGCAGGCACGTGCACATCGCCATCGCCACACGCCAACTCCGCCTGGGTT
TTACAAAGTCGTTGCCTTAATGCATGTGGACAGGAACTCCCTGAGGTCGCCCCATGC
CCCCTGGCTGTGCCAGGTACGGACGCCCTGGACCCTGCGAACAGGTGGGGGGGGCG
AGGGGCCCAAGGGACGGGCTCCAGAGACACGCGCAGGGCAGGAGGGGTCTCACGG
AGGGGTCTCGCACTGAGGCGCCCAGAGCTGGTGGTCCCGCTGGACGCCATCCCTCTG
CCCGGGATCCACACGGCCCACGTGTGCCCGCCATGCCCGCGCCCCACGCCATTGCAG
TCTGCCATCCTCTGGCCGTGACGGTGGCTGCAGCTTCCCCATTTGCGCCGTTGCCTCT
GGCTGTCTGCACTTTTGTTCATGCTCCAAAGAACATTTCATAATGCCTTCAGTACCGA
CGTACACTTCTGACCATTTTGTATGTGTCCTTGTGCCGTAGTGACCAGGCCTTTTTTT
GGTGGATGTGTTACCCCGCACACTTCAATCTCAACTTTGTGCACCGTCCATTTTCTAG
GGATAGACGCCCAGGGAATGAACTCTAGTTTTCTAACAGATTAGCTGAGATATTAAC
TTACTCACACGGACAGGTTGATGCCAGAGCCGTAAGAATGCGCCAGTGCGGGTTTGC
GGGGGACTTCGGGTGTGGGGTCCTGCGGCCGCGATGGCCGTGGAAGGTTCTGGGGA
TCCCTGCTGCCACGGGGACGAGTTCGGACGCCAGGTGGACCTGTGCACTCAGTAAAA
CGCAGTGATTCAACCTGGA
(Source: NCBI Reference Sequence: XM_017027842.2)

In various embodiments, a KCNQ2 miRNA transcript comprises the sequence provided as SEQ ID NO: 3042.

(SEQ ID NO: 3042)
CTCCTAGGCCCAAGCTGTCCTCCTGCCTCGGCCTCGCCAAGCTCTGGGAGTACAGGC
GTGGCCGCCGGGCCTGGCCTGTTTGCTTCGGATGTTTCGTGTAAGTGGAGTTGTCATC
GTGGTGCCGTTTTGTGCCTGGCTTTCCTTGCCGAGCCTGCCCTCAGGTTCCTTCGTGT
TTAGGGTACTCCTTCATCCTCTCCGTGTCTGGGGGCCGCTGCCTCGTCCTTCCTGTGG
CCGCACGGACGGACCTCAGTTTATCTACCCACCCACCAACTGGATGTTGGGTTGCGT
CTGCCGTGAGGCTGCTGTGGACACGCGGTTCCTCCTGGTTTTCTCCTGCCTCGTGCTG
TCTGTGTTTTCCACCATCAAGGAGTATGAGAAGAGCTCGGAGGGGGCCCTCTACATC
CTGGAAATCGTGACTATCGTGGTGTTTGGCGTGGAGTACTTCGTGCGGATCTGGGCC
GCAGGCTGCTGCTGCCGGTACCGTGGCTGGAGGGGGCGGCTCAAGTTTGCCCGGAA
ACCGTTCTGTGTGATTGACATCATGGTGCTCATCGCCTCCATTGCGGTGCTGGCCGCC
GGCTCCCAGGGCAACGTCTTTGCCACATCTGCGCTCCGGAGCCTGCGCTTCCTGCAG
ATTCTGCGGATGATCCGCATGGACCGGCGGGGAGGCACCTGGAAGCTGCTGGGCTCT
GTGGTCTATGCCCACAGCAAGGAGCTGGTCACTGCCTGGTACATCGGCTTCCTTTGT
CTCATCCTGGCCTCGTTCCTGGTGTACTTGGCAGAGAAGGGGGAGAACGACCACTTT
GACACCTACGCGGATGCACTCTGGTGGGGCCTGATCACGCTGACCACCATTGGCTAC
GGGGACAAGTACCCCCAGACCTGGAACGGCAGGCTCCTTGCGGCAACCTTCACCCTC
ATCGGTGTCTCCTTCTTCGCGCTGCCTGCAGGCATCTTGGGGTCTGGGTTTGCCCTGA
AGGTTCAGGAGCAGCACAGGCAGAAGCACTTTGAGAAGAGGCGGAACCCGGCAGC
AGGCCTGATCCAGTCGGCCTGGAGATTCTACGCCACCAACCTCTCGCGCACAGACCT
GCACTCCACGTGGCAGTACTACGAGCGAACGGTCACCGTGCCCATGTACAGTTCGCA
AACTCAAACCTACGGGGCCTCCAGACTTATCCCCCCGCTGAACCAGCTGGAGCTGCT
GAGGAACCTCAAGAGTAAATCTGGACTCGCTTTCAGGAAGGACCCCCCGCCGGAGC
CGTCTCCAAGCCAGAAGGTCAGTTTGAAAGATCGTGTCTTCTCCAGCCCCCGAGGCG
TGGCTGCCAAGGGGAAGGGGTCCCCGCAGGCCCAGACTGTGAGGCGGTCACCCAGC
GCCGACCAGAGCCTCGAGGACAGCCCCAGCAAGGTGCCCAAGAGCTGGAGCTTCGG
GGACCGCAGCCGGGCACGCCAGGCTTTCCGCATCAAGGGTGCCGCGTCACGGCAGA
ACTCAGAAGAAGCAAGCCTCCCCGGAGAGGACATTGTGGATGACAAGAGCTGCCCC
TGCGAGTTTGTGACCGAGGACCTGACCCCGGGCCTCAAAGTCAGCATCAGAGCCGTG
TGTGTCATGCGGTTCCTGGTGTCCAAGCGGAAGTTCAAGGAGAGCCTGCGGCCCTAC
GACGTGATGGACGTCATCGAGCAGTACTCAGCCGGCCACCTGGACATGCTGTCCCGA
ATTAAGAGCCTGCAGTCCAGGATAGATATGATTGTGGGTCCCCCGCCCCCTTCAACT
CCCCGGCACAAGAAGTACCCCACCAAAGGACCCACGGCCCCTCCGAGAGAGTCACC
CCAGTACTCACCTAGAGTGGACCAGATCGTGGGGGGGGCCCAGCGATCACGGACA
AGGACCGCACCAAGGGCCCGGCCGAGGCGGAGCTGCCCGAGGACCCCAGCATGATG
GGACGGCTCGGGAAGGTGGAGAAGCAGGTCTTGTCCATGGAGAAGAAGCTGGACTT
CCTGGTGAATATCTACATGCAGCGGATGGGCATCCCCCCGACAGAGACCGAGGCCT
ACTTTGGGGCCAAAGAGCCGGAGCCGGCGCCGCCGTACCACAGCCCGGAAGACAGC
CGGGAGCATGTCGACAGGCACGGCTGCATTGTCAAGATCGTGCGCTCCAGCAGCTCC
ACGGGCCAGAAGAACTTCTCGGCGCCCCCGGCCGCGCCCCCTGTCCAGTGTCCGCCC
TCCACCTCCTGGCAGCCACAGAGCCACCCGCGCCAGGGCCACGGCACCTCCCCCGTG
GGGGACCACGGCTCCCTGGTGCGCATCCCGCCGCCGCCTGCCCACGAGCGGTCGCTG
TCCGCCTACGGCGGGGGCAACCGCGCCAGCATGGAGTTCCTGCGGCAGGAGGACAC
CCCGGGCTGCAGGCCCCCCGAGGGGAACCTGCGGGACAGCGACACGTCCATCTCCA
TCCCGTCCGTGGACCACGAGGAGCTGGAGCGTTCCTTCAGCGGCTTCAGCATCTCCC
AGTCCAAGGAGAACCTGGATGCTCTCAACAGCTGCTACGCGGCCGTGGCGCCTTGTG
CCAAAGTCAGGCCCTACATTGCGGAGGGAGAGTCAGACACCGACTCCGACCTCTGT
ACCCCGTGCGGGCCCCCGCCACGCTCGGCCACCGGCGAGGGTCCCTTTGGTGACGTG
GGCTGGGCCGGGCCCAGGAAGTGAGGCGGCGCTGGGCCAGTGGACCCGCCCGCGGC
CCTCCTCAGCACGGTGCCTCCGAGGTTTTGAGGCGGGAACCCTCTGGGGCCCTTTTCT
TACAGTAACTGAGTGTGGCGGGAAGGGTGGGCCCTGGAGGGGCCCATGTGGGCTGA
AGGATGGGGGCTCCTGGCAGTGACCTTTTACAAAAGTTATTTTCCAACAGGGGCTGG
AGGGCTGGGCAGGGCCCTGTGGCTCCAGGAGCAGCGTGCAGGAGCAAGGCTGCCCT
GTCCACTCTGCTCAGGGCCGCGGCCGACATCAGCCCGGTGTGAGGAGGGGGGGGAG
TGATGACGGGGTGTTGCCAGCGTGGCAACAGGCGGGGGGTTGTCTCAGCCGAGCCC
AGGGGAGGCACAAAGGGCAGGCCTGTTCCCTGAGGACCTGCGCAAAGGGCGGGCCT
GTTTGGTGAGGACCTGCGGCCTTGGGTCCCGGTGGGGTTTCCGGGCAGCTACAGGCG
GGTGTGGCCGGCCGCTGTGCGTGGCCTCTGCCTTCACACCTGACCTGCCCGGCGGGC
TTTCCTGTTCCCCACCTCAGGGGCGCCCAAATACAGAGCTATTGGTTGGCGTCTTCTC
CCTGTACCTTCTGGGATCTGAGGGCTCTTTCCATGGAAGCCAGCCCCGAGGTGGAGA
CCTTCGCCTGCAGCCGAGGAGCGGGTGGGGCCTGGGAACCAAACTGGAGCCAGAGT
GGACGTCCAGCCCTCTGGTCTTGGCCTCCAGAGGGAGGGCCTGGCTCACGGTGGGGC
CAGGGAGCCGGCTCCAAAGGGTCTTCAAAAAGGGGGTCCTTGGGGGCTCCAGCTGC
CTCGCCCTGGCCTTTCTGTGGGTGCGTGAGAGCCAGCAGCACCCCAGCCTTGGAGAC
CGGGGGGGCAGGACCCCAAGTCCTCCCCTCTCTCCTGACTGCCCTGGCCGGGTGCCG
GCACTGCGAGACCCACCTGGTGAGCAGGCCTCACAGTTCTTAGCCAGGGCCCCACCT
CGCCTGTGTCCCACCAGTGCCCCGACAGACCTGGGGCAGGGCTGGGCCATGATGCA
GCGGGCCAGGATAGCCTCCACCGTCAGCACAGGGCCGCCCTCCCCGCCTTTCCGGAG
GAAACCACTCCCACCTCAGCCCAGCTGTGCGCCCTCCCTAGCTCTCCTGCCCCCTGG
AGCTGATGGCCCCTTCTCCACTGACCGATTCCTTAGCGGGGCCTCTTGGGGTCTCGG
GCCTCGGGTGCACCGTCCCATGCCCGTCCTGTTGTGGGCACCGTGGCCCTTGGGGCA
GGCGGCTCTAATGCGGGAGCGAGTCCCTAGCTCCAGACTTAAGAACCAGACCCCGG
GAGCATCTGGCATTTGGCGTGACGGCGTCGCAGGCGGGCCTGGGCTCCCTGGAGAGT
GGCCTCCCTGGGAGTGAGCAGGGCTGGGGTCGTGGGCGCAAATACTCCTGCAGAGC
AAGTGCAGGGGAGTCCTGGGCCCGTTTCTCCTCCACCTGCGTTTTCAGTGCACTTGGC
TTGGCTGGGAGGTCCTGAGGCCCTGAGGCCAGCAGGGGAACCAGTCCTGAGGGAGA
GGACTTTGAAAGCAGCATTTGAGGGTCGTACGCCCCTGGCTGGTGGGGGTCCTGGCG
CTCAGGGTGTTCGGGGAGCCATGTCTGGCGTCCATTGTGGGGAGCTGCTGCCCTGGC
CTCTCTGCCTACCCCCAGCCCGGCCAGGGCACTCCCAGGCCCTGTCGCCATTGAGGT
GCCTCCGCTGGGCTGTCTCCTCACCCCTCCCTGTGCTGGAGCCTGTCCCAAAAAGGT
GCCAACTGGGAGGCCTCGGAAGCCACTGTCCAGGCTCCCACTGCCTGTCTGCTCTGT
TCCCAAAGGCAGCGTGTGTGGCCTCGGGCCCTGCGGTGGCATGAAGCATCCCTTCTG
GTGTGGGCATCGCTACGTGTTTTGGGGGCAGCGTTTCACGGCGGTGCCCTTGCTGTCT
CCCTTGGGCTGGCTCGAGCCTGGGGTCCATGTCCCTTTGCCGTCCCGTCATGGGGCA
GGGAATCCATAGCGGGGCCCACAGGCAGGGGTATGAGTGCGTCCCACCCAACGCAG
CACCAGCCCCGGCCACCGCTCCCCGTGTCCCCAGTTCCGTCTCAGCTACCTGGACTC
CAGGACCCTGGAGAAGGGAGACCTGGCAGTGGAGGGAGGCTGTGCTGTGTGTCCCC
CTGCAGGTGTGACCCCGCTGCTCTTTCCTCCCCCGCCAGGTGTGGCCCCGCCTGCTCT
TTCCTCCCCCACCAGTATGGCCCCACCTGCTCTTTCCTCCCCCCCCAAGGTGTGGCCC
CACCTGTTCTTTCCTCCCCTGCCGAGGTGTGACCCCACCTGCTCTTTCCTCCCTCCCA
GTATGGCCCCACCTGCTCTTTCCTCCCCCGAGGTGAGGCCCCGCCTGCTCTTTCCTCC
CATGGGGCCGCTGAGGCATGAGCACCTGGGCACAGGTTGGGGCTCTGCAGGATGAG
GAAGACAGGCCAATCCCTTCCCTCCCAGAAGCTGGCCGCCCAGCAGGAGGGACTGA
GGCCAGACTCATGTCCAGCAAGGAACGTGTGGTGTGTCCCCTGGGAAGTCTCTGGGC
CCTGGGAAGAGGGAAGGTGCACGTCCTGGGATGGTTGCGGGGCCCTGTTTTGGGAG
ACAAAGGGGTAGAGGGTCTGTCTTGGGCCCCCCCAGACTCTAGCCTGAGCAGTGCA
GCCACCTACTGCCCCACCTCAGAGAAGTGCAGCGGGAAGGAGGCTGGAGGTGGTGC
GGCGCTGCCTCGGGTGTCTGCGTGAATGAGCGTGGCCAAGGACCAGTGCCACCTCAT
GGCAAAGAGCTCCCGCAGTGTTTGTTAGAGTGCACATCCCTACGTGCCCACTGGCAC
ACACACGTGCTCACATACATGTCCGCATACAGGCGTACACATGCACGCTTGCACACA
TGCACACAGACCACATAGCACACATGTGCACTGACCACACCTGTATAGACCATGCAC
AGTACACATACGTGCATACACATGCCTGCATACAGGCATACACATGCACGCTTACAT
GTACACGTGCACAGATCACACACATGCACACACGTGTAGCTCACACACAGTATACA
CATACACAAGTGCACAGACCACACACAGCACTAACACATGCACACACAAAGTGCAT
AGGCCACACAGCACATGCACACAGGTGCACAGACCACACAGCACACACAAGTGCAC
AGAGCACACTGCACACATGCACACACACACGCGTGCATGCACACTCCTCGCACTTCC
AGCCTTGGAGCCCTTCTGTCTCTGGTCTTTCTCTTTGACCCTGCTGAGTGTAAGCTGC
CTGGGGAGGGGCTACAAGGAGTAATTGTGGCTTTAGGGGTCGTGGTGATGCTGGAA
TGTCAAGCGCCGTCGTGGGGTATCCGACTGTCCGGGCTCCTGGTCCGCAGTGGCAGA
GCGCCAGGCAGAGCCCAATCAGGGTCTCGTGCTGCCCTTCCCTCCCACAGCCTGGCA
GCCATCCAGAGGAGGGGCTCTACCAGATGCCAAGGTGCCCCGGTGTCTGTATGGGTG
TCCGGTTGGGTCCTGTGTTTGGTCTGCCCTGGAGGTGCTGGGCCCTCCTGGGATGGGT
GGCTCAGCCTCGAATCCCAGGCCCCAGCCCAGGCAGGTGCTGCTGCCTGTTGTGGTT
TCCTGGCCCAGCTTCTCCTTCTCCCTCTGCATAAAATCACAGTCCGTGAGTCTTCCAG
CTGCCACCACGGCTGGGACACGCTGGGGGAGGGCTCCTCCCATGCCTCCTGCACACA
GCCGTCTGAGCAGGGCAGGTGCCCAACACCCCCCACCGGAGGACACGCTGCCCCTC
AGCGATGCCCCTACCTTTTGGGGGGCCTCGTCTCAAGCCCCCCCTTGGAGGCTGAAA
TCACCCCAGGCACTGTGAGGGCTTCTCCAGGGGGCACCCTTTGAGCTGTGGGTCTGA
TCACCCCAAGTCCCGCCCGGAGGAGAGGCACAGCCAGGGCGTGTGGTTTAATGTTTG
CCCCCTTCGGGGCTGGAGGTCTCAGTGTTTCTAGATTCCAGACCCTGCTGCCAGAGA
GACCTGCTGCCGGAGAGAAGGGGAGGAGGACTCCAGCTGGGCTCGGTCCCCCACAG
TCAGGGACCCCCATAAAGGACACCCCCTTCTCTCTAGAAAGAGCTGGGCTCTCAGCT
ATTTCTAGTTGCTTCCCAGAAGCCGAGGAGCAGAAGGAGCTGTGAGAGCTTTGCAGA
AACGCCCTTGTCCCCGCCCTCCTGAGCTATGAATGCCGTACAGAGCAGAGGCTGGGG
CATTGGCAAGATCACAGGTTGATGCTGCACAGCCCCATTGACACAAACCCTCAAAGC
AGACGTGAGAGGGACGGTTCACAAAGCTCGGACCTGCCGTGGAGGGTGCCCGGCAG
ACGTGGCGTGAGAGGGACGGCTCACGAGGCTTGGACCTGCTGTGGAGGGTGCCCAG
CAGACGTGGTGTGAGAGGAACGGCTCACGAGACTTGGACCTGGTGGAGGGTGCCCA
GCAGACGTGGTGTGAGAGGGACGGCTCACAGGGCTTGGACCTGCCATGGAGGGTGC
CCGGCAGACGTGGTGTGAGAGGGATGGTTCACAGGGCTTGGACCTGCCATGGAGGG
TGCCCGGCAGATGTGGTGGGAGAGAGATGGCTCATGAGGCTTGGACCTGCCGTGGA
GGGTGCCCAGCAGACGTGGTATGAGAGGGATGGCTCACGAGGCTTGGACCTGGTGG
AGGGTGCCCGGCAGACGTGTGAGAGGGACGGTTCACAAGGCTTGGACCTGCCATGG
AGGGTGCCCAGCAGACGTGGTGTGAGAGGGACAGCTCACGAGGCTTGGACCTGCTG
TGGAGGGTGCCCAGCAGGGGGCTGAGCTCTGAGGGGTGGGTGCTCAGTGCACGGGT
GCCCCCAGTGTCCTCTGATCCTGTCCGGTGCCTCCCCCAACCCCCACACCCATGCAG
AACTCCCAGGTCACATGCACGTATGTCCAGGGCATGGGGGTGGCGTGAAGAGGCCT
GGTCAGGGCCTTTAGGGGCTGCAGGACGGAATGGCCGCCTGGGGAGCCTGTGTGGC
TGTGCCGGGCAGCCATCCTGCATTCCCACCCAGCGCGCAGTCTCCACCTCGGCCCCA
GCAAAGCGCTAAGCAGCCGGAGAGACAGCCAGGGCGGCTTCCTGAAGGATGTGGGA
TGGTGGACTCCGGGGTCGAGGGAATACGCAGGTTCCTGTCCCTCCGGGAGACCTAGA
GAAGCTGCACACCCAGGAGCTTTCCATGACCCGGGAGCATGAGTGAATGGGGGTTC
CAGTTTGCTGAACTTTGCCGTCTTGTAAGGGTGGGGGCTGACGGCCGACCCTGGGAG
GAGGTGACATCGCCGGGGGAGGTTGTGGGCAACGGTGGAGGAGGAGAGACGGGAG
GGGACCATTTGGGATGGAGGGGCCTCTTCAGAGTTTTAAAAGGCGTTTGTGGGGTGG
AGTTGAGTGTGCTCTGGGCTTGGACACTTGCCGTGGTGCCCCTGGCTGGCCGAGGAG
ACTGGCTCTGGCCAGGGGCCCCGTCCTGAGAGGTCCTCAGCGTCTGACTCTCGGCCA
GGCGCCAGCAAGGAGGGGCCGGTCCCCGGGGCTACCAGGCAGGCACGTGCACATCG
CCATCGCCACACGCCAACTCCGCCTGGGTTTTACAAAGTCGTTGCCTTAATGCATGT
GGACAGGAACTCCCTGAGGTCGCCCCATGCCCCCTGGCTGTGCCAGGTACGGACGCC
CTGGACCCTGCGAACAGGTGGGGGGGGCGAGGGGCCCAAGGGACGGGCTCCAGAG
ACACGCGCAGGGCAGGAGGGGTCTCACGGAGGGGTCTCGCACTGAGGCGCCCAGAG
CTGGTGGTCCCGCTGGACGCCATCCCTCTGCCCGGGATCCACACGGCCCACGTGTGC
CCGCCATGCCCGCGCCCCACGCCATTGCAGTCTGCCATCCTCTGGCCGTGACGGTGG
CTGCAGCTTCCCCATTTGCGCCGTTGCCTCTGGCTGTCTGCACTTTTGTTCATGCTCCA
AAGAACATTTCATAATGCCTTCAGTACCGACGTACACTTCTGACCATTTTGTATGTGT
CCTTGTGCCGTAGTGACCAGGCCTTTTTTTGGTGGATGTGTTACCCCGCACACTTCAA
TCTCAACTTTGTGCACCGTCCATTTTCTAGGGATAGACGCCCAGGGAATGAACTCTA
GTTTTCTAACAGATTAGCTGAGATATTAACTTACTCACACGGACAGGTTGATGCCAG
AGCCGTAAGAATGCGCCAGTGCGGGTTTGCGGGGGACTTCGGGTGTGGGGTCCTGC
GGCCGCGATGGCCGTGGAAGGTTCTGGGGATCCCTGCTGCCACGGGGACGAGTTCG
GACGCCAGGTGGACCTGTGCACTCAGTAAAACGCAGTGATTCAACCTGGA
(Source: NCBI Reference Sequence: XM_017027843.1)

In various embodiments, a KCNQ2 mRNA transcript comprises the sequence provided as SEQ ID NO: 3043.

(SEQ ID NO: 3043)
GTTCTTGGTGATTGGTTTCATTTTTTGCTTAATCATTTGCACATTGTGATGCAGAGCT
AAATCGCCAGGGTTTTTGGGGAGATTCTTGGGAAAACGTTCCGTGTTTTCTGAGCCTT
TTCTAAGGAACGCCCCGGCGGGGAGCTGCGCTCCTGAAGGGCTTCACAATGAGGGG
ATCCACTGAGCCCCAACGTGACTGTCCTCAGGGGAGCCCTTGAACCCCCAGATAGCC
ATGCCTGCAGTGGCAGCCACGCCTCCTGGGTAAACCGAGACCCACCTCCAACTGGG
GGACTGGCTGGCATGGCCAAACGTCGCCGATTGGTTTGCAAAGCGGGACAACTGCA
CGTTTTCCTCTCTCAAATGTGTGTGCTTCACACCCACGCCCTTTCCCGGGGTCACGTC
CTTCCCAAGCAGGGCTTCTGCGGCGTCTGAGGCCACATGTATTATTGTTTTGTCAGCA
TGAATACTGCAAAAAATAATACATCTTTTAAGACATGAACCCAAACTGTGGCTTTGG
AAGGAAAAGGTATTTTTGGAGGAAAAAAAAAACAGGGATTTTTTTCCTTCAGAAAT
AGGGAAGTGTTTTCTTCTCCGGACGGTGGAGACGATGGTGCACCTTCCTCGCTGGCC
TTGCTTGGGCGCAAGCTCTGCCGCTGGGGACTGCACACAACACCCTGGGTGGTTGCA
GTGCCTCAGTTTCCCTGCTCCCGGCCGTGTGCTGTTGCCGGCGCCCAGGACTAACTGT
GCTCTCCTCATTTCCAGTAAAGGCAGCCCGTGCAGAGGGCCCCTGTGTGGATGCTGC
CCCGGACGCTCTAGCCAGAAGGTCAGTTTGAAAGATCGTGTCTTCTCCAGCCCCCGA
GGCGTGGCTGCCAAGGGGAAGGGGTCCCCGCAGGCCCAGACTGTGAGGCGGTCACC
CAGCGCCGACCAGAGCCTCGAGGACAGCCCCAGCAAGGTGCCCAAGAGCTGGAGCT
TCGGGGACCGCAGCCGGGCACGCCAGGCTTTCCGCATCAAGGGTGCCGCGTCACGG
CAGAACTCAGAAGAAGCAAGCCTCCCCGGAGAGGACATTGTGGATGACAAGAGCTG
CCCCTGCGAGTTTGTGACCGAGGACCTGACCCCGGGCCTCAAAGTCAGCATCAGAGC
CGTGTGTGTCATGCGGTTCCTGGTGTCCAAGCGGAAGTTCAAGGAGAGCCTGCGGCC
CTACGACGTGATGGACGTCATCGAGCAGTACTCAGCCGGCCACCTGGACATGCTGTC
CCGAATTAAGAGCCTGCAGTCCAGGATAGATATGATTGTGGGTCCCCCGCCCCCTTC
AACTCCCCGGCACAAGAAGTACCCCACCAAAGGACCCACGGCCCCTCCGAGAGAGT
CACCCCAGTACTCACCTAGAGTGGACCAGATCGTGGGGCGGGGCCCAGCGATCACG
GACAAGGACCGCACCAAGGGCCCGGCCGAGGCGGAGCTGCCCGAGGACCCCAGCAT
GATGGGACGGCTCGGGAAGGTGGAGAAGCAGGTCTTGTCCATGGAGAAGAAGCTGG
ACTTCCTGGTGAATATCTACATGCAGCGGATGGGCATCCCCCCGACAGAGACCGAGG
CCTACTTTGGGGCCAAAGAGCCGGAGCCGGCGCCGCCGTACCACAGCCCGGAAGAC
AGCCGGGAGCATGTCGACAGGCACGGCTGCATTGTCAAGATCGTGCGCTCCAGCAG
CTCCACGGGCCAGAAGAACTTCTCGGCGCCCCCGGCCGCGCCCCCTGTCCAGTGTCC
GCCCTCCACCTCCTGGCAGCCACAGAGCCACCCGCGCCAGGGCCACGGCACCTCCCC
CGTGGGGGACCACGGCTCCCTGGTGCGCATCCCGCCGCCGCCTGCCCACGAGCGGTC
GCTGTCCGCCTACGGCGGGGGCAACCGCGCCAGCATGGAGTTCCTGCGGCAGGAGG
ACACCCCGGGCTGCAGGCCCCCCGAGGGGAACCTGCGGGACAGCGACACGTCCATC
TCCATCCCGTCCGTGGACCACGAGGAGCTGGAGCGTTCCTTCAGCGGCTTCAGCATC
TCCCAGTCCAAGGAGAACCTGGATGCTCTCAACAGCTGCTACGCGGCCGTGGCGCCT
TGTGCCAAAGTCAGGCCCTACATTGCGGAGGGAGAGTCAGACACCGACTCCGACCT
CTGTACCCCGTGCGGGCCCCCGCCACGCTCGGCCACCGGCGAGGGTCCCTTTGGTGA
CGTGGGCTGGGCCGGGCCCAGGAAGTGAGGCGGCGCTGGGCCAGTGGACCCGCCCG
CGGCCCTCCTCAGCACGGTGCCTCCGAGGTTTTGAGGCGGGAACCCTCTGGGGCCCT
TTTCTTACAGTAACTGAGTGTGGCGGGAAGGGTGGGCCCTGGAGGGGCCCATGTGG
GCTGAAGGATGGGGGCTCCTGGCAGTGACCTTTTACAAAAGTTATTTTCCAACAGGG
GCTGGAGGGCTGGGCAGGGCCCTGTGGCTCCAGGAGCAGCGTGCAGGAGCAAGGCT
GCCCTGTCCACTCTGCTCAGGGCCGCGGCCGACATCAGCCCGGTGTGAGGAGGGGC
GGGAGTGATGACGGGGTGTTGCCAGCGTGGCAACAGGCGGGGGGTTGTCTCAGCCG
AGCCCAGGGGAGGCACAAAGGGCAGGCCTGTTCCCTGAGGACCTGCGCAAAGGGCG
GGCCTGTTTGGTGAGGACCTGCGGCCTTGGGTCCCGGTGGGGTTTCCGGGCAGCTAC
AGGCGGGTGTGGCCGGCCGCTGTGCGTGGCCTCTGCCTTCACACCTGACCTGCCCGG
CGGGCTTTCCTGTTCCCCACCTCAGGGGCGCCCAAATACAGAGCTATTGGTTGGCGT
CTTCTCCCTGTACCTTCTGGGATCTGAGGGCTCTTTCCATGGAAGCCAGCCCCGAGGT
GGAGACCTTCGCCTGCAGCCGAGGAGCGGGTGGGGCCTGGGAACCAAACTGGAGCC
AGAGTGGACGTCCAGCCCTCTGGTCTTGGCCTCCAGAGGGAGGGCCTGGCTCACGGT
GGGGCCAGGGAGCCGGCTCCAAAGGGTCTTCAAAAAGGGGGTCCTTGGGGGCTCCA
GCTGCCTCGCCCTGGCCTTTCTGTGGGTGCGTGAGAGCCAGCAGCACCCCAGCCTTG
GAGACCGGGGGGGCAGGACCCCAAGTCCTCCCCTCTCTCCTGACTGCCCTGGCCGGG
TGCCGGCACTGCGAGACCCACCTGGTGAGCAGGCCTCACAGTTCTTAGCCAGGGCCC
CACCTCGCCTGTGTCCCACCAGTGCCCCGACAGACCTGGGGCAGGGCTGGGCCATGA
TGCAGCGGGCCAGGATAGCCTCCACCGTCAGCACAGGGCCGCCCTCCCCGCCTTTCC
GGAGGAAACCACTCCCACCTCAGCCCAGCTGTGCGCCCTCCCTAGCTCTCCTGCCCC
CTGGAGCTGATGGCCCCTTCTCCACTGACCGATTCCTTAGCGGGGCCTCTTGGGGTCT
CGGGCCTCGGGTGCACCGTCCCATGCCCGTCCTGTTGTGGGCACCGTGGCCCTTGGG
GCAGGCGGCTCTAATGCGGGAGCGAGTCCCTAGCTCCAGACTTAAGAACCAGACCC
CGGGAGCATCTGGCATTTGGCGTGACGGCGTCGCAGGCGGGCCTGGGCTCCCTGGA
GAGTGGCCTCCCTGGGAGTGAGCAGGGCTGGGGTCGTGGGCGCAAATACTCCTGCA
GAGCAAGTGCAGGGGAGTCCTGGGCCCGTTTCTCCTCCACCTGCGTTTTCAGTGCAC
TTGGCTTGGCTGGGAGGTCCTGAGGCCCTGAGGCCAGCAGGGGAACCAGTCCTGAG
GGAGAGGACTTTGAAAGCAGCATTTGAGGGTCGTACGCCCCTGGCTGGTGGGGGTC
CTGGCGCTCAGGGTGTTCGGGGAGCCATGTCTGGCGTCCATTGTGGGGAGCTGCTGC
CCTGGCCTCTCTGCCTACCCCCAGCCCGGCCAGGGCACTCCCAGGCCCTGTCGCCAT
TGAGGTGCCTCCGCTGGGCTGTCTCCTCACCCCTCCCTGTGCTGGAGCCTGTCCCAAA
AAGGTGCCAACTGGGAGGCCTCGGAAGCCACTGTCCAGGCTCCCACTGCCTGTCTGC
TCTGTTCCCAAAGGCAGCGTGTGTGGCCTCGGGCCCTGCGGTGGCATGAAGCATCCC
TTCTGGTGTGGGCATCGCTACGTGTTTTGGGGGCAGCGTTTCACGGCGGTGCCCTTGC
TGTCTCCCTTGGGCTGGCTCGAGCCTGGGGTCCATGTCCCTTTGCCGTCCCGTCATGG
GGCAGGGAATCCATAGCGGGGCCCACAGGCAGGGGTATGAGTGCGTCCCACCCAAC
GCAGCACCAGCCCCGGCCACCGCTCCCCGTGTCCCCAGTTCCGTCTCAGCTACCTGG
ACTCCAGGACCCTGGAGAAGGGAGACCTGGCAGTGGAGGGAGGCTGTGCTGTGTGT
CCCCCTGCAGGTGTGACCCCGCCTGCTCTTTCCTCCCCCGCCAGGTGTGGCCCCGCCT
GCTCTTTCCTCCCCCACCAGTATGGCCCCACCTGCTCTTTCCTCCCCCCCCAAGGTGT
GGCCCCACCTGTTCTTTCCTCCCCTGCCGAGGTGTGACCCCACCTGCTCTTTCCTCCC
TCCCAGTATGGCCCCACCTGCTCTTTCCTCCCCCGAGGTGAGGCCCCGCCTGCTCTTT
CCTCCCATGGGGCCGCTGAGGCATGAGCACCTGGGCACAGGTTGGGGCTCTGCAGG
ATGAGGAAGACAGGCCAATCCCTTCCCTCCCAGAAGCTGGCCGCCCAGCAGGAGGG
ACTGAGGCCAGACTCATGTCCAGCAAGGAACGTGTGGTGTGTCCCCTGGGAAGTCTC
TGGGCCCTGGGAAGAGGGAAGGTGCACGTCCTGGGATGGTTGCGGGGCCCTGTTTTG
GGAGACAAAGGGGTAGAGGGTCTGTCTTGGGCCCCCCCAGACTCTAGCCTGAGCAG
TGCAGCCACCTACTGCCCCACCTCAGAGAAGTGCAGCGGGAAGGAGGCTGGAGGTG
GTGCGGCGCTGCCTCGGGTGTCTGCGTGAATGAGCGTGGCCAAGGACCAGTGCCACC
TCATGGCAAAGAGCTCCCGCAGTGTTTGTTAGAGTGCACATCCCTACGTGCCCACTG
GCACACACACGTGCTCACATACATGTCCGCATACAGGCGTACACATGCACGCTTGCA
CACATGCACACAGACCACATAGCACACATGTGCACTGACCACACCTGTATAGACCAT
GCACAGTACACATACGTGCATACACATGCCTGCATACAGGCATACACATGCACGCTT
ACATGTACACGTGCACAGATCACACACATGCACACACGTGTAGCTCACACACAGTAT
ACACATACACAAGTGCACAGACCACACACAGCACTAACACATGCACACACAAAGTG
CATAGGCCACACAGCACATGCACACAGGTGCACAGACCACACAGCACACACAAGTG
CACAGAGCACACTGCACACATGCACACACACACGCGTGCATGCACACTCCTCGCACT
TCCAGCCTTGGAGCCCTTCTGTCTCTGGTCTTTCTCTTTGACCCTGCTGAGTGTAAGC
TGCCTGGGGAGGGGCTACAAGGAGTAATTGTGGCTTTAGGGGTCGTGGTGATGCTGG
AATGTCAAGCGCCGTCGTGGGGTATCCGACTGTCCGGGCTCCTGGTCCGCAGTGGCA
GAGCGCCAGGCAGAGCCCAATCAGGGTCTCGTGCTGCCCTTCCCTCCCACAGCCTGG
CAGCCATCCAGAGGAGGGGCTCTACCAGATGCCAAGGTGCCCCGGTGTCTGTATGG
GTGTCCGGTTGGGTCCTGTGTTTGGTCTGCCCTGGAGGTGCTGGGCCCTCCTGGGATG
GGTGGCTCAGCCTCGAATCCCAGGCCCCAGCCCAGGCAGGTGCTGCTGCCTGTTGTG
GTTTCCTGGCCCAGCTTCTCCTTCTCCCTCTGCATAAAATCACAGTCCGTGAGTCTTC
CAGCTGCCACCACGGCTGGGACACGCTGGGGGAGGGCTCCTCCCATGCCTCCTGCAC
ACAGCCGTCTGAGCAGGGCAGGTGCCCAACACCCCCCACCGGAGGACACGCTGCCC
CTCAGCGATGCCCCTACCTTTTGGGGGGCCTCGTCTCAAGCCCCCCCTTGGAGGCTG
AAATCACCCCAGGCACTGTGAGGGCTTCTCCAGGGGGCACCCTTTGAGCTGTGGGTC
TGATCACCCCAAGTCCCGCCCGGAGGAGAGGCACAGCCAGGGCGTGTGGTTTAATG
TTTGCCCCCTTCGGGGCTGGAGGTCTCAGTGTTTCTAGATTCCAGACCCTGCTGCCAG
AGAGACCTGCTGCCGGAGAGAAGGGGAGGAGGACTCCAGCTGGGCTCGGTCCCCCA
CAGTCAGGGACCCCCATAAAGGACACCCCCTTCTCTCTAGAAAGAGCTGGGCTCTCA
GCTATTTCTAGTTGCTTCCCAGAAGCCGAGGAGCAGAAGGAGCTGTGAGAGCTTTGC
AGAAACGCCCTTGTCCCCGCCCTCCTGAGCTATGAATGCCGTACAGAGCAGAGGCTG
GGGCATTGGCAAGATCACAGGTTGATGCTGCACAGCCCCATTGACACAAACCCTCAA
AGCAGACGTGAGAGGGACGGTTCACAAAGCTCGGACCTGCCGTGGAGGGTGCCCGG
CAGACGTGGCGTGAGAGGGACGGCTCACGAGGCTTGGACCTGCTGTGGAGGGTGCC
CAGCAGACGTGGTGTGAGAGGAACGGCTCACGAGACTTGGACCTGGTGGAGGGTGC
CCAGCAGACGTGGTGTGAGAGGGACGGCTCACAGGGCTTGGACCTGCCATGGAGGG
TGCCCGGCAGACGTGGTGTGAGAGGGATGGTTCACAGGGCTTGGACCTGCCATGGA
GGGTGCCCGGCAGATGTGGTGGGAGAGAGATGGCTCATGAGGCTTGGACCTGCCGT
GGAGGGTGCCCAGCAGACGTGGTATGAGAGGGATGGCTCACGAGGCTTGGACCTGG
TGGAGGGTGCCCGGCAGACGTGTGAGAGGGACGGTTCACAAGGCTTGGACCTGCCA
TGGAGGGTGCCCAGCAGACGTGGTGTGAGAGGGACAGCTCACGAGGCTTGGACCTG
CTGTGGAGGGTGCCCAGCAGGGGGCTGAGCTCTGAGGGGTGGGTGCTCAGTGCACG
GGTGCCCCCAGTGTCCTCTGATCCTGTCCGGTGCCTCCCCCAACCCCCACACCCATGC
AGAACTCCCAGGTCACATGCACGTATGTCCAGGGCATGGGGGTGGCGTGAAGAGGC
CTGGTCAGGGCCTTTAGGGGCTGCAGGACGGAATGGCCGCCTGGGGAGCCTGTGTG
GCTGTGCCGGGCAGCCATCCTGCATTCCCACCCAGCGCGCAGTCTCCACCTCGGCCC
CAGCAAAGCGCTAAGCAGCCGGAGAGACAGCCAGGGCGGCTTCCTGAAGGATGTGG
GATGGTGGACTCCGGGGTCGAGGGAATACGCAGGTTCCTGTCCCTCCGGGAGACCTA
GAGAAGCTGCACACCCAGGAGCTTTCCATGACCCGGGAGCATGAGTGAATGGGGGT
TCCAGTTTGCTGAACTTTGCCGTCTTGTAAGGGTGGGGGCTGACGGCCGACCCTGGG
AGGAGGTGACATCGCCGGGGGAGGTTGTGGGCAACGGTGGAGGAGGAGAGACGGG
AGGGGACCATTTGGGATGGAGGGGCCTCTTCAGAGTTTTAAAAGGCGTTTGTGGGGT
GGAGTTGAGTGTGCTCTGGGCTTGGACACTTGCCGTGGTGCCCCTGGCTGGCCGAGG
AGACTGGCTCTGGCCAGGGGCCCCGTCCTGAGAGGTCCTCAGCGTCTGACTCTCGGC
CAGGCGCCAGCAAGGAGGGGCCGGTCCCCGGGGCTACCAGGCAGGCACGTGCACAT
CGCCATCGCCACACGCCAACTCCGCCTGGGTTTTACAAAGTCGTTGCCTTAATGCAT
GTGGACAGGAACTCCCTGAGGTCGCCCCATGCCCCCTGGCTGTGCCAGGTACGGACG
CCCTGGACCCTGCGAACAGGTGGGGGGGGCGAGGGGCCCAAGGGACGGGCTCCAGA
GACACGCGCAGGGCAGGAGGGGTCTCACGGAGGGGTCTCGCACTGAGGCGCCCAGA
GCTGGTGGTCCCGCTGGACGCCATCCCTCTGCCCGGGATCCACACGGCCCACGTGTG
CCCGCCATGCCCGCGCCCCACGCCATTGCAGTCTGCCATCCTCTGGCCGTGACGGTG
GCTGCAGCTTCCCCATTTGCGCCGTTGCCTCTGGCTGTCTGCACTTTTGTTCATGCTCC
AAAGAACATTTCATAATGCCTTCAGTACCGACGTACACTTCTGACCATTTTGTATGTG
TCCTTGTGCCGTAGTGACCAGGCCTTTTTTTGGTGGATGTGTTACCCCGCACACTTCA
ATCTCAACTTTGTGCACCGTCCATTTTCTAGGGATAGACGCCCAGGGAATGAACTCT
AGTTTTCTAACAGATTAGCTGAGATATTAACTTACTCACACGGACAGGTTGATGCCA
GAGCCGTAAGAATGCGCCAGTGCGGGTTTGCGGGGGACTTCGGGTGTGGGGTCCTG
CGGCCGCGATGGCCGTGGAAGGTTCTGGGGATCCCTGCTGCCACGGGGACGAGTTC
GGACGCCAGGTGGACCTGTGCACTCAGTAAAACGCAGTGATTCAACCTG
(Source: NCBI Reference Sequence: XM_017027845.1)

In various embodiments, a KCNQ2 transcript is a KCNQ2 pre-mRNA transcript. In various embodiments, a KCNQ2 pre-mRNA transcript comprises a sequence provided as SEQ ID NO: 3044.

AGGCGCCGAGGTGCGCGCGGAGCGAGGTGGCCGCAGCGTCTCCGCGCGCGGCCCAAGCCCG
GCAGGAGTGCGGAACCGCCGCCTCGGCCATGCGGCTCCCGGCCGGGGGGCCTGGGCTGGGG
CCCGCGCCGCCCCCCGCGCTCCGCCCCCGCTGAGCCTGAGCCCGACCCGGGGCGCCTCCCGC
CAGGCACCATGGTGCAGAAGTCGCGCAACGGCGGCGTATACCCCGGCCCGAGCGGGGAGAA
GAAGCTGAAGGTGGGCTTCGTGGGGCTGGACCCCGGCGCGCCCGACTCCACCCGGGACGGG
GCGCTGCTGATCGCCGGCTCCGAGGCCCCCAAGCGCGGCAGCATCCTCAGCAAACCTCGCGC
GGGCGGCGCGGGCGCCGGGAAGCCCCCCAAGCGCAACGCCTTCTACCGCAAGCTGCAGAAT
TTCCTCTACAACGTGCTGGAGCGGCCGCGCGGCTGGGCGTTCATCTACCACGCCTACGTGTG
AGTGGCCGGCGGGGCCCCCGTGGCGACCCCCATGGCGACCCCCATCGGCGACCCCATCGGC
GACCCCGGCCAGGCTGGCTGCGGCGGGTTTGGCTCCCTGCCCCTGGGTCTGGGTCCGGGGCC
GGCGCTCCCCGGCGGGAGTGCTGGGCCGAGACCGGAGAGCGATTGTACAGCGCGCGGGCAG
GAAGGATTCCGGGGTCGGGGCCGCTCAGGTCGGGGTGGGGGCTCCAGGCCCGCAGGACAGA
GACGTGCGGCCGCCCCAGCCCCCTCCTCAGCCTGGGAGGCCCCTCCCCGCGGCCTCTGCGCC
GAACAAAGGGCCGGCCGGGGAGGGACGGCGGCAGCGCTGGGTCTGGACTCTCCGGGACTCT
CGGGGACCCGCGGGGCTGGGGCCGCCCGACCAAGCGAGGGCGGGGGGGAGGGGTCTGGCCT
GTCCTCCGCCATCCTCCCGAGCCGCTCTCCCTCAGACTAGGGTGAGCTTCTGGGTTCCTGAGA
TGGGGGAGGGGCCCTCCCATGACCCCCCCACCCCAGCCCGGGCTGAGCGCAGCTGTCTGTCC
TGCCCCCTCGGCGCCGACGCCCCTCCCTGGCCCCGCGAACCCCCTCAGCTCCTGCGCTCAGCT
CCTGCGCTGTCCCATCTCTGCAGCCGCCGGCCGCCCCCGTGCACATCCCGGGCTCAGGACCC
CTCCTTGGCAGAGCGAGGCCCGGGGGAGGGGCTGCGGCAGACCTGGGACCCCCAGCCCTGG
GCCTGGAAGGGGCTGTCCCCTCTTCCCGGGCGAGGCTCAGGGAACGGCTGCCTCCTGGGGCA
GGGCTGGCGGGGGGCGGCCGAAGCCTGCGTCCGTGTGCACCTGGGTGTTCGCATGTGGAATT
GCACGGCCCGCTGCGTGTGCGGCCTGTGCGGCCGGTGTGCACAGCCTCCCCGCGCCAGGGCG
CTTGCTCTCCCATCCCAGCTGAACAAGGGACCCGCTGAGCTCCGGGACCGGCCATTTTGTGT
GAGAGGCATTTTGGGATGGGAGTGAGGGGCAGACTGGTCACAAGCTGGTCGGGCGCCCACC
TGGGCCGAGAGGGGTGTGTGCCCCACGAGTGTGCCCCAGCCTGCGTGTGGCCTTGAGTGTGC
CCTTAGCAGACCCTCCGTGGACTGTGATATGAGAGGGTTCTGGGGTAGGGAGAAGATCCCAG
AGGGAAATGCCAGGCCGCTTACAGGGAGGGTGTCTCCGTGGAGGGCCCCTGGGCACCCAGC
CTGGGACTCCCCAAGCAGAAGCTTCTCCGACCTCCTGGGTCCTGGAAGCCCTGTGGCCCCCA
GGAGGTGAGTCTAGCTTTTCCAGTCCAGGGTTCATTCCCGAATCCCCAGATGAGCTTCTGTGT
TCTCAGGAATGGCCTCCCCTGATGGCCCCCACCTGGCTGGGGGCCAAAGGGGCTCTTGGTGG
GCCGGTACTGGTCAGCCAGGTCCCTGCCCAAGGTGACTCCCCGGAGGAGGGTAGGATAGAA
CCCACCCCCACAACCCTCCTGCCCTGACAGCCTGGCCAGCGCCCCGCTCTGCGTCTGACCCA
GGGTTGGTTCTGCTTTGAGTCCATGATCCAAGGGCCCAGCGTGGCTGTTCCTGAGGTTTCCCG
TTGGGTCAGCAGCTCTCCGCATGGCCGCCCCCTGCCTGGGCCTGCGTGGGTCAGCCCCTCTGT
CTCTCCTTCCTGTCTCTGCCTCCTGAGAGCAGCAGTCCAGAACTGGGGCAAAGCATGGGGAC
ACTTGCACATGTGGGGTCTGAGCCGTCTCTTCCTGACCACCCCCAGTCCGGACGGGCCCCCTC
TAAGATAGAGCAGCAGCATGCGGGGGCTTCAGGCGAGACCCTCAACGTCTGAACAGCCCCG
CCTTCCAGCCTGCACTAGGCCTCTCCTCAGCTCCTTTGCTGTGGGCACGGAGCTCCCTGAACC
TCCCTGAGCTCCCAGGCTCTCCTCAGCTCCTTTGCTGTGGGCAGGGGTGGCTTGGACTCAGGG
AGGCGGCCCCTTTGGGAGGGGGATTTTTTCTAAGCGGCGGGGCTGGAGAGCAGGGTTTCTGG
TCCTGCCTCGGGTTGGACACTGGGGGCTGGTCTCAGAACCTCTGCAAACCTCAGTTTTCCGTC
GGTAACGTCGGCACAGCTGCTTCTCAACCCAGGGCTTTGTGAACACCACTGCTGGGGGGCAT
CACACTCTTCCAGGCCGGCCCAGTGCCCTCTGTGAAAGGGGATGTGGCTCAGCACTGCTGGT
GCAGCTCACCGCCCGGCTCCCAGACCCCTGGTGTGGTGTTTGCTGCTGTCCCCGTGGCCCAGC
CCATCCTTGGAGTGCAGCCCCCACCTCCACTTGGGAGGCCTGATGGCCCCATGTCCTCTGGCC
CGGCATGGACTGGCCCGTGTGGCGTGTTAGGCAGCATTTGGTCTGTTTGTTCATCAGCTAAAC
GGAGACGGAGCTCAGTTGGGCCTGGGGCAGCCGGGAGTGGGGGGAAGGCCAGGGCTCCTTG
TGCTGCCGCACCCAGCTGTGGCTGAATACAGGTCCCCAGGCCCACCTGCCTTTTGCCTATTGC
CACCAGTGTCTGGCTTCGGAGAGGTCAAGTTGTGAGAACCTCTCTGGGCATCCCTGTTGGAC
CCGGGGTAGCCACTGTCTGGGGTAGAGAGGGCGGTGGCTGCTGCCCCATGGCCCCAGGGCTT
GGTGGGCGTCAGGCCAGGCCCCTGCTGCCCCCGCTCTGGGGAGGTGGGGACGTCCAGCACTG
AGTTAGAGAGGGACTTCATCCACTGTTGCTCCTCTGTGGGAGCCGTGGCCTCTGCTCGCACTG
CCTGGGTGCTTTCCCAGAGGCTCTGGAGACAGGGCCTTCTCTGAGTTAGAAGGGCGTCAGAC
CTGCTCTCTGCCTGGTCGGGGGGGGGTGCTGGGCCACCTCTCCCAGACCTCCCAAAGGGAGT
GTGAGCTGCAGAAATGGCCCGAACTAGCAGATGATGTGCCCCTGGGGCTGCCGCAGGGGAC
TCTGGGCCAGATCACCATGGGGGGCCCTTCGGAGCCTGAATCCCGGGCTCCAGCCTCTCTCC
TGCAGCCCATCTGCGCATAGGACTGGAGCCGGGCACTGCTCCACCACGGAGTTGAACCTCCT
CGTCCAGGATCCCCGCGTGGGTTGCTGGGTGTGGGAAAGGGAGCTCCGCTCTGTTCCCGCTG
GGTGCGGACCTCAGTTACCGCTTAGTTACAGCCTGCCAGAGGGTCACGCAGCCTGGTTCCTG
TGGATGGGGAAACTGAGGCAGAGGAAGGTTGAGTGAGCTCCAGAGTTGTCCTGCTTAACTG
AAAGGGGGGGTCCCAGCCTGAGATGAGAGCTTGGGGGTGCAGATTGCTTGATCTTGCCAGCA
GCCCAGCCACGTGGAGTGGCCAGAGTGAAGCTGTGTTTAAAATAGGCCAGATGGGAAATCT
GGCACCGGGGAGGGCCTGTCCCTGGGGCTCGCTGTGCCATCCTGGCTTTTGCGCCAGGCCTC
AGCTGGGGTCCAGGGTTTTAGGGGCCTCTGGCCCTGGGCTGCTTGACCAGGTGCTGTGTCCC
ATTTCACTGCAGGGAAGGGCCTGGGACGGAGGGCCTATGTTCAAGTCCCCCGGTGCCGGGGC
CCAGGCGCTTCCCCTCACAGTCCTGGAGAACTCGGGTGGTGCTGAGCACAGGCCGCTGGGCC
TCTTTCCCACCCCTTCATTCAGCTCTGGGGCTGGTGTGAGCCCTCGAAGGGCTGGCTGGGGTG
GGCAGGCGCCTGGCACAGTGGTGGGGTTCAGACACTCTCTTCTTGGTCAGGCTGCTTTGAGG
GGTGAGTGCAGGCCTGGGCTTTACCCAGCACAGAGCGGGGCCCTGGCAGGACCAGGGTCCA
GTCTCTGCTGACGGAGCATTGTGTGCCGAGAAAGAGACTCGAGTGCTTGAACGGCCACATTC
CCCTTCTTCCCACAGCCTCCTGTGTAGGAAATCTTATGGAAATTTCCCAAAAGGTGAAGAAG
CCTCGGAGAGTTGTGGTTTCTGGGGAAACCTGGATTTCTCTGGTTCCTGGACCCCTTGGGAGG
TCTCGGGCTTCCTCGCAGGATTTCTGCCCCTCACCAAATAATTTAAAGTGAAATTACTCTGGA
GTGATAAGACATTGTACATGATGCTGCTTAGTTACACAGTGGAGTCAATCTCTGTGCACATTC
TGTGTCTGTGAGAGGATAGGAGCTAAAGGATAAGCAGAAACTCTCCTCCTGACCATTCCACA
GTGAGAGCAGAGAAAATCTGTTTTATTAGAAATCACTGCAAGCCAATCTCTGCAGAGACGCG
GTCTCTCCAGAGGTGCCGGCGCCCTGGATATGGGCACTGCTGGGCACTAAGGATTCTCGGCC
CCCAGGATGCTGACCACGTGGGAATCCTGTCTCCAGTTTTGAAAATGCTTAAATCCTGGAGT
GTTTCAGAACTGGGGTCACTTCTCACGGCACAGGCTGAGTCCAGTGGTGGAGAGTGGCTTAA
CATAGTGATTTTCCCAGTTTGTTTTAGTCAATAAACAAATGAATCTATGGATCTCCGTTAGTC
CCTGTGCTCCTGACACGTCCATGGCCCCTGTGTGCACACTCAGCAAGTCCGTCTGGCAGCTGC
TCGCAAGAGCACGGGGCTAGCCTCTGCCCTGTAGGAGTGACATTCCATAGCCTCACAGGAGC
TGGGCTAGCCTCAGCCCCATAGGAATGACATTCCACAGCCGTAGAGGAGCCAGGCTAGCCTC
AGCCCTGTAGGAGTGACGTTCCATAGCTGCACAGGAGCCGGGCTAGCCTCAGCCCTGTAGGA
ATGACGTTCCGCAGCCGTAGAGGAGCCGTAGAGGAGCTGGGCCTGAAGCTGCTTCCAGTGTG
GGGAGATGAATCTGCTACACAGAGTGGGTTCCCTCCTAGGAAAATGGGCAAGTCTGGAGGA
CCAAGGGGGACCTGTGGGGGCCTTGAATATGGGGAGAAGGGATATGGGATGGCCGAGCTGC
CTTGCCCAGAGGCAGGGGAGGGGCAGCCTGTGCCCTGGGCCCTCCGAAGGTCCACTTGAGGT
TTGACGGGAGGCTGGGGCCAGCGGCCAGGCCGTGTCCTGTTAAAGGCTCAGGCGGGAGCAA
TGTGCCCGTTTTGGGAGGATGGGGGGCAAGGATGGTGGGGTGATGCCTGCGATGTCCCCCGG
GGGAGACCTTGGCCTTGTGTCCGACATCTTGTCATCACAGACAGAGCCCAAGCGTGTTACCT
CACGGGGGGCTGTCTTAACTATTTTCCAGTGTGCATGCCAGGCAGCTCTGAGACGGGAAACT
GCTCGTGTGTGCGCGGTCTGTGGACGCAGCCCCGCTCCCCTCTGTGCTTCCGCCCGTGTGAGA
CGTGCTCCTGTGTGGCCGTGACCTGTGAAGACCCTGCGTGACCCGTGGGGCGTGAGGCTCGG
GGAGGGCAGGGCTGGGGCTCTGGCCGGGGGCGTCATCCTGAGACTGGCCAGCGTCGGGCTC
CTAGACCCGAGTTTCCTTGTCTGCGTGGTGGGGGTAACCTGTCTGCATCTGTGTCACGTGATG
GCCGCCGGGATTCAACGTGTGAGTCACTCCACACGATGCCTGATGCGTGCGATGTTCCCCAA
GCTCGTAGCTGTCATCGCCGTGACCGTGAGGCTCCTTCAGCAGAGTGGAAGGCCTCACCCCG
GACGGAGCGTGGAGGCCAGGAGGGAGGATGGCTGGCCGGTCCGGGACGGCGTCGCTCTGCC
CCTCCGGCCGATTCTCAGTCCCATTGGTCAGCGGCTGGGCCGTGACGGAGAGGCCTGCGCTG
CTGTTGAGGGCCAGGTCGTGGGTTGAAGTGCGGGGCCCGGGCTCGAGCGTCGGAAGCCTGC
GGGGGTCGTGGGTTGAAGCGCGGGGCCCCGGCTCGAGCGTGGGAAGCCTGCGGGGGTCGTG
GGTTGAAGCGCGGGGCCCGGGCTCGAGCGTGGGAAGCTTGCCTGTTTGTCGTGTGGGACCGG
CCGCGGAGATGCCGGCTGTTCACTTGGAACGAAGGTGCCTGCCCTGTGTGTGCCGGGTAGTT
TGGGGGATTGTCTTCCTGTTCGTGGCTTCCGGCGTGTTCCCAGCATCCTGAGGGTTGAGGCTT
GGAGGACAGGAATGATCTTCGGCTTGAGAAACCACTGTGGGCAGGTTTTACCAGGGAACCCC
TTAAGCTGTGAAAGAGCTGCAGTGCAAGCTTCTAGTTTCCACGCAGACCGCGCCAGAGAACC
CGCCGGGCCGCCTCGGGACGGCAGCAGAGGACCCGCCGGGACGCCTCAGCACCGACACGCG
CCCAGCCATGAGGCCGCCTTTAGCACGGAGCGTCGAGCGGAAACGGCGGTGCCCGGAACGC
GGATGCCCTTTGCAGGGCTGGAGCTCAGCTCTGCCTTCCGACAACACTTGGCTGACAGCTCT
GGGAGTCTCAAAACGGTGCCTTTGTGGAGGGGAGGGCCCTGGACATCCTCAGAGGAGGAAG
CGCGTCCTGGGTAGGGTGGGCGGCACCTGTTCAACACGCCTAACAGATGCAAGGTCGAAGG
AGGAGGGGCCTGCAGATCTGAGCTTGTCGCCGAGGAACTCTCTGGAAATGGCTCCCGCTGGC
CCCTGCGTGCGACCCCTACGCTCACCAGCCCGTGTGCCGAGCTTTCTCTGTTGATTTCGGAGT
GGGTTCTGGTTCCTGCCTCCCTAGTGGGCTCTGAGTGGCAGGGCCCTGCCGACCGCTGGCCTC
AGCAGCGGGTGATGGAGTGTGGGCCCTGCAGGAGGAGGCTCTGTCCTTGATGGCTCCTGGGT
GAGGGGGTGTGAGGGAGGCGGCCAGCACAAGGCGGGTGGGCCCTGAGAAGCCCCTCTGTTT
CCCCTTTAGCCTGAGAAAGCTGTACCCACAGAGCTGGAGGAGGGAGTGGCTCAGGATGCTGC
TTAGCAAATAGGTTGGTCTGGAGTCTGGGAAATGGAGGGTTAGACTTTTTGTTTTCTGAAGCT
GGGATGGGTCGTGCTTTTAATTCGTCCAGGAGAACATGGGGCTGAAGAGCTCTCTGCTCTTG
TGGCCCCTGATGGGAGTGCTCTGAGGTCTGGCAGGCCACAGAGCTGCTGGGACGGCCTCAGC
CCCAAACCTGCGCCTGTCGCTTCCCGGGGCCAGGCCAGAGGTTTCCCAGTGAGAGCTTTGGG
GTCGACATTTCCAGGAGGAGAAGGCACTTCTGAAATAGCGTGCCCTTGGTGAGCTTGTGTGG
CTTTTCTTTCTCTGTAACCTGCTCTGAGTTGTCCCATTGAGTGTTCTGGGGATGTGGGCATCTG
GAGCCTGTGGAGAGGACGGGACTGGGGGTGCGTGGGGGAGACGCCCCTGCCAAGGACAGGG
GAGACCCCCTCAGTCCCACCTGGGAGAGTCTGTGCGCGGGGCTGTGGTTGATTGGCTGGCAA
GTTTGAATGCGGATCAATGTGCCACTTGTTTCTAAGTCAGCAGGGCCCCTGCCCCCACACACT
GAGTGGCTGGATTCTGGGAGCCTTTTCCTGTGGAGAGTGGAGCTGGAGTGTGGGGCGCAGGG
CCATGGCCCTGTTCCAGCGTCGGGGAGCTAGTGTGCAGGTGCTTTCTCCTGTTAGCAGTGGGC
CTGTGCTGTGCTCCCTGTGGGGTTAGGCGGCCCCGGTGGGAGCTTCAGTCTTGAAAGGGGCT
CATAGCCCTGGGGTGCAGGCTGCTGTGCGTGGGGGGCATCACACGCAGGTCCCCAGGATGAT
GTGTGGTGGAGGACGGGGCGACGGGAGCTCTCCACAGAGCTTCATGTCCCTTCTGTTGTCTA
AACCACAGACCCCGTGCCAGGTGAGGGGCTGGGGTGGGTGAGGCTAAGGTACCTGCCCCGA
GGCCTCGGGGCACTTGGCGGCCCCTCCACCCCCACAGGAGACATCGGCTTGAGTGGAGAGG
CCAGCCTCTGAGAAGGCTTCTCTCTGGCCACCAGCTGCCCAGAGCTCCCAGCCCAGGGCCAG
CAGCTTGTGCTGGCCCCAGACAGGGGATGCTGGGCGGCCTCTCGCAGGGGGAGTGGTGGTTT
TCAGACAGTGGCTGCCTCGCTTTGGGGATGAGGATGTTTTCCAGACGTGCTCTGGAACTGAC
ATTAGGCTCTGCTGCCTACCCAGCTTGAACAAAATTAGGTCTTTCATTTTTTTGGGTTGCTGG
GAATGGCTTTGTGGATGCATAGGAGAGCATGGTTGCTCCAGGAAGTCCGGCGGGGAGTGGG
TTGAACAATCGGAGTCTCCCATCCCTGAATCATCTCAGTGGGGGCGGACGGGCAGCTCCTGG
GGGTGTGGCAGGGGCTGGGCTCCAGGCTGCCCAGCCTGCAGGGAGCCTCCTTCTAGATTCTT
CCTCTGTATCTGTGGCGTGGAGGCATCTGCTGCCTCTGTCTGCCCCCGCCTCATCCTCCAGGT
CTTCTCTAGTTTTGTTCCTCCCCATGGAGCCCTTACCCGTGACGTAAGCTGAACACCTGAGAA
CTTTCGAGCTTGCTTTTCTGTTTAGGTTTGGAAAAAACATGAGAAGGGTGAGTGGGAGCAAA
AGGCCAGTTTCTGCCTGGCTCCCTGTCCTGGGAGGAGGTGGGGAGAAGCCTCGGGCAGGGAT
GAGAGGTTGGGATGTCTGCCCTGAGGGGCTGTGGGCGGGAGGCCGGAGGCGCCTGGTTAAT
GTGTTTCTGCCCCTGTGTGCGGGTGCAGGGTTCTAAGCTGGGACTCAGGGTGCAGAGGGCAC
GTGTGTGGGACTTTAAAGTTTGTCATGGAGGCTGAGCCCTGGGGAAGGGCAGCAGCACTCGG
CCGGCTGGGCTTCCTTCTCCTGGAGTAGGGTGGTCAGGGAGGCAGGGATATCGGGGAGCGCC
ACCCCAGGACGGAGCAGGAGGCAGCCTGGAGTCCCCTTTGTAGACCAGATAAGGCCAGGCA
GGGCCTGATGTCCGTATTCTATTATTTATTTATTTTAAGTGTGGGGGTCTCGCTCTGTGGCCCA
GGCTGAGTGCAGTGGTGTGATTGTAGCTCACTGCAGCCCAGAACTCCTGGGCCTGAGCGATC
CTCCTGCCTTGACCTCCTGAGTAGCTGGGAGATGACCCCCTTCTTACCCGTTCTTCCAGAATA
GGTCAAATTCACATGACATGTGCACACACACACACACACACACACACACACACAGAAAAGA
CACCCACGGAAACCCTGAAATATATAAAATGATGTCCAGCCAGTATGATTTGCCCCCTCTGC
GCTGCCCCATCTCCGTACCGGGTCAGCCATGGCCCATCTCCTGGAGCCGTTCTCTGCATGCGT
GCCACGGTACAGCATGTGTGTGTCTGTGTGTGTTCACTCACTTCTTTTCACATGTGTGGAGTC
ACTCCGTGTCCTGCCTTGTGCGTGTGGCCCCCTCTCCCTGCATGTCCCCCTCTAGGCTCCGTC
GGGCGCTCACGACTTCTCTGACCGGTTTGCAGTGATGGACCTTGAAGCAGGTGCTAGGACTT
TGCCGCTGCAAATGGTGCTGCGTGGGGCTCCATGCACATCTTCACGCAAGGTGCAGGTCGGC
AGGATGAATTCCTAGAAGCTGCCTGGTGGCTGTAAAAATGTTCTCATCCCAAAGGACCAAAT
ATGCAGAGAAGTTTAACACTCACTTTTCCTGTCATTGAAAAGTTACATTTTTGTGGAAACCCG
TCCAGGCTGGAGACAGAAGGTGTGTGCAGGGAGAGGAGGCTCTGGGGCACCACTCTAGGCT
GCCTGCTGCTCAGGTGGTTGATGTGGGGGCAGATGCCCTGTTCCTTGGGGTAGGTGCCACCT
CCTCCCTGCTCCCTGGGGTAGGTGCATTTTCCTCCCTGCTGCCTGGGGTAGGTGCCGCCTCCT
CCCTGCTCCCTGGGGTAGATGCAGCCTCCTCCCTGCTGCCTGGGGTAGGTGCCACCTCCTCCC
TGCTGCCTGGGGTAGGTGCAGCCTCCTCCCTGCTGCCTGGGGTAGGTGCTGCCTCCTCCCTGC
TGCCTGGGGTAGATGCAGCCTCCTCCCTGCTCCCTGGGGTAGATGCAGCCTCCTCCCTGCTCC
CTGGGGTAGGTGCAGCCTCCTCCCTGCTGCCCCGGGGTAGGTGCAGCCTCTTCCCTGCTCCCT
GGGGTGGGTGCAGCCTCTTCCCTGCTCCCTGGGGTGGGTGCAGCCTCCTCCCTGCTCCCTGGG
GTAGATGCAGCCTCCTCCCTGCTGCCTGGGGTAGGTGCAGCCTCCTCCCTGCTGCCTGGGGTA
GGTGCTGCCTCCTCCCTGCTGCCTGGGGTAGATGCAGCCTCCTCCCTGCTCCCTGGGGTAGGT
GCAGCCTCCTCCCTGCTCCCTGGGGTAGGTGCAGCCTCCTCCCTGCTGCCCCGGGGTAGGTGC
TGCCTCCTCCCTGCTGCCCCGGGGTAGGTGCAGCCTCTTCCCTGCTCCCTGGGGTGGGTGCAG
CCTCTTCCCTGCTCCCTGGGGTAGGTGCAGCCTCCTCCCTGCTCCCTGGGGTAGGTGCAGCCT
CCTGCCTGCTGCCCGGGGTAGGTACAGCCTCCTCCTCCCTGCTGCCCGGGGTAGGTGCAGCC
TCCTCCTCCCTGCTGCCTGGGGTAGGTGATATTTCTGCCCTCCTACCTGATTAGGCTCTGGGG
CCACTCTTGGGGATGGGGGAGGAGGGCCGGCCCCCGGGGTGCTGGCCATGGTCCTGACCAG
GAGCTGCAGTTGCGGCTCCTCGTGGGCCAGAGGATCCCCTTGTGTGATTCTTGTTTGGCATCC
GCCCAGGCCATAGAGTCAGGGGCTTGGCTCACCTCTGGGCAGCTATGGGGAAGGGGCTGGT
GTTTGGGAGGTGCCCGAGGAATGATCGGTGGGGGGTGGCTGCTGGGGCAAGGTACACGACC
GGCTTCTCTCCTTTACAATTAGCACTGAGCCAGCTTATCTGAGACCATCCCTTCCCACGGGTG
CCATTTAATTAGCCAAGAGCTGAGTTAAGATCACGTGAAGAAAGTTGGGGGGGCCTTGGCCT
GAGCGGCGGGTGGACAGGGCTGCGTCTGTCAGGGTGAGGGGGTCTGGCGGCCCCTGCCGCC
CTCGTTTCTTAAATAAACGTTTGGTCGTGGCTTATTTTCTTCTGACTGCAAAGTAAGCACATC
CACCGCAGGAGACTGGGAAAGCGTGGAAAACAGGGAAGGACGTTTGCTCTTCCTGGTTTCTT
TCCAGGTGTCTGCGCCCGCCTGTGCGTTTCTAAACAGAATAGGATTCAGGCGGTCCCGTCCC
GCTGTGGTGTTAACGGCATACTGTCCACACTTCTCATGGGCAAGATGAGTCTCGCGTGCCAG
GTAGTTGGCTTGTCTCTGACCTGGGGCTGCCGCAGCACCTGTCCAGCCACCTGTGGACATGG
ATGACGGCAGAGTCCCTGTGTTGTGTGGTGTCCCTGCGTGGATGTCGACACCACAGCTGTCCT
GGGGACGTGTCCACATGAGGCTTGGGGAATCCACATGTGTCCCATCCAGGGCTGTCCTGCAG
GCCCCGGGTCTGCCTGAATGTGATTAGGTGATGAGGCCACCCTGGCCAGGAAGGCTGGTGAG
GGGAGAGGGAGGACGCAGGCTGGCCCAGGAGGGGCTTGTGTTGGAACGAGATTCTTTCTGT
GGGGTGAGCCCTCCGTCGCCGGAGGTGTGCAAGTGGAGGCCGAGAGAGAGCACTCACTAGG
ACGTTTGTAGGGGGCCAGGGACTGGAGCTGTGAGCCGTGGACCAGCTGAGGGCACTGGCCT
GCTTGGGAGGGGGTTAGGCGTGGCTGTCTCAGGGGGCTGTGGGGGGGAAGGCCTGCTGGGA
GCCTCGGTCAGGAGTGCCACTGTTGGGGTGCCCTCAGCACCTGAATGGGTTTCGGGTGGGGC
TGACAGGCAGCTTGAGCCCATGAAGGGAGTGTGGGATGTCTCAGGAGGAGCCCCCTTGTTGA
GGGTGGGGGGAGCCCCTCTCCACCCCAGCTCCCCTGGGACCTCATGAAGCCTTTGCTCCCCA
AAGCTGCTAAGGGCTTGGGTAAGGCCAGGGAGGGACCTGCAGGGTCAGGAGCCCAGGCCAG
GCCAAGCAGGGGTCCCCAAAGGTGCTGGGGATGGGGGTGTGCATGACAGGGAAGCTCCGGG
GCTCGGGTTTGGGGAGGAGTAGGCTGGGCTGGGATGGGGACACTGGCCAGGGCCAGAGCCA
AGCTTCAGCCACAGAAGAAGGGGCGATTCTACAGCTCACAAGGTCCCCGGGTGCCGTCTGCT
TCCTGTAGTGTAAACGCACCTGTCCACATTCCAGCCTTCCTACCGAGACAACAGCACCATGG
ACTGGAGGAGTCTGGAGGGGACAAGGTGTGGTGAGTGTTAACTCTCCCCACCCAAAGCTATC
CTATTCAGAGAGAAACTGAGGCACAGGTGGGTGGACACCCTAGGCACAGAGCCACTGATGG
GCAGGCAGGTTACCTGTGTGCGCTTGCTGTCCCAGACCTTGACAACACGGTTTATTTTGTTTT
TTAAAAATAGAGGTTAAATTATCCTAACATGCAGATAGCATTTTAAAGCGAGTAATTCAGAG
GCGTTTAGAGTGGAGAATTTAGTGATGGTCTGCCGCCACCAGCCCTATTTGAAAACTTGTCA
CCCCATGTGGACACTCTTGCCCTCTGCCATCGTGTGCCCCACCCTGTCCCTGGCAGTTGCCGG
TCTCCTGCCTGTCTCTGTGACGTGCCTGTTATTTTATTTTATTTAATTTATTTATTTTTGAGGCA
GGGTCTCTTTTCGCCCAGGCTGGAGTGCAGTGTCTCAATCACAGCTCACTGCAGCCTGGACCT
CCTGGACTCGAGCCATCCTCCAGCCTCCGTCTCCCGAGTCGCTGGGACTGCAGGTGCCCCAC
CACATCTGGCTAACTTAAATCATTTGTAGAGATGGGGTCTCATTGTGTTACCCAGGCTGGTCT
CGAACTCCTAGGCCCAAGCTGTCCTCCTGCCTCGGCCTCGCCAAGCTCTGGGAGTACAGGCG
TGGCCGCCGGGCCTGGCCTGTTTGCTTCGGATGTTTCGTGTAAGTGGAGTTGTCATCGTGGTG
CCGTTTTGTGCCTGGCTTTCCTTGCCGAGCCTGCCCTCAGGTTCCTTCGTGTTTAGGGTACTCC
TTCATCCTCTCCGTGTCTGGGGGCCGCTGCCTCGTCCTTCCTGTGGCCGCACGGACGGACCTC
AGTTTATCTACCCACCCACCAACTGGATGTTGGGTTGCGTCTGCCGTGAGGCTGCTGTGGACA
CGCGGTGAGTGTCTCTGGACATTTCGTCATTCCTCTTGGGACGAAGCCTGCGTCACATGTGGC
CAGGTGCCCTGGGTGGGCCGTGCCCTCCTCATCTCAGCCCCCCAGACCCACACAGGCCCCCT
CCCGGAGCCCTCGCCTTGCTCTTCCCGCTGTGCGGAAGGTGGTGCAGTGACGCCTGAGTGTG
GGCCCCAGGCCTAGCTGCCAGCCTGGGCTCTCGAGCAGGTTCAACTTGGCCAGGGCAGGGTC
TTGGCTGCCGCCTGCCTGGATGTGGGGAGACGCCTCTGTGCAGGTTCTGGGCTGGGCACTTG
GGGCCACTGTCTGAGCAGAGGATGTGGGGTCCTGCCTCCGGGAGGGCGAGCAGACCGCTGC
TCTGGGGGGACAGTGCTCGGTCAGGCTCAGGGCCCTGGAGAGAATACACCAGGGAGACTTG
GGGGCTGCCCGGGGAAGGGATGGTCGGGCAGCCTTTCAGAGAAAGTGACCTGAGATTGAAG
CCTGGGGGATGAGGGGGCGGCTCTGGGACCTGCGGGTAGGAGAGGATTCCAGGCAGCAGGA
AGTGCACGTGCAAAGGCCCTGGGGCAGGGACGGCGTTTGCCGAGGGCAGGAGGGGAGCAGG
GTGGATGGGTCGCGTGGGGTCCACCATGAGGCACTGCAGCGGCCTCGAGGGCCAGGCCAGG
GTGGGGTCTATCTTCAGGGCCTGGGGCTCGCCTATGCTGGCTGCCATTCCCACCCAGCTTCTC
CACATGCTTCTGGGATCAAAGTGGCTGGTACAGGGGCGCTTCTCTGCGGGCTCAGTGGGTTG
GGATGGGGCAGTGGAGAGTGGGTAGCCGGGGCATGCCGGGAGGAAGAGTAAAGTCCAGGG
GACCCCAGGCCAGGGGCTGCGAGCCCAGGGAGGTTGCGGGGTCGGGGAGGGCGGGCCAGG
ACCTCTGCATGGGGCTGGGCTGGGCACTTTGGCCAGCCCTGCTTGGTGCACCGGCTGGGTCT
CTGTCCAGCACCTGTGGTCTGGGTGGGACAGGTGACTAATGGAGAACAAATGAGCCCTGCGG
AGCTTCCGTCTCCAGGGACCGTGAGCAGGCGCTGCGAAGTCACGAGGCAGTGCCCGGGGAG
CTGATTTAACCACGTGCTGAGCCAGCGGGTTTCCTGCCAGGGCCTGGGAGTCTGCCCACAGC
TGCAGCTCCCAGAGCCCCTGGGGTGCATGGCGGATGCCCCCTCTTTCTGGGCCGGGGCGAAG
GCCCTGGAAGGACCTGTGGTTGCTGCAGCAATGCAGGGGGCTGGGGGTCCCTGTGGGCCTTG
GGCGTCTGCTGCGGGAGTGGTTAGGGAGCAACGTGCAGGTGGGCAGGACCCCCGCCTCCAG
CTCCTCCACTCCTGTTCCCGAGGGAGCCACAGCTGGCTGAAGGAAGTGGCCATCCTGGGGCC
ATGGTCAGTGTGGGGCATCGAGAGCCCCCTGAGAGCGCCTCCCTGCCCAGGATGTTGGAAGG
AGGGCTGTAGGTGCCAGGTGGGTGCAGGCGGGCATAGGCAGGCCCAGGCCGTTTGGAGGCC
GTGAGCATCAGGCTAGCAGCTGGGCCTTCTGAGGACCTGAGACTTCCTGACCTGTGTCCTGT
CAGGGTCCAGTTGAGCACCCAGTGTTTGGGAGGCTCGGTCCCACCTGGGTCACCTCCCGTCC
CTGCCCCATGGCCTTAGGCTCAGGTGGGTGCTGCCGGAAGCTCCGAATGAGGGTGGACCCTG
GCCTGGCAGGTACAAGCCACGCAGTGCTCAGCATGAGGCGGCTCCACGCGAGAGGTCTCTGT
GTGCAGCTCTGCAGGAGCGTGGCCCTTGTCCCCTCCAGCCCCGGCGGTCTGCAGTGATCCAT
CACGCTTGGTTCATGCCCGGTTCACGCCTGGACTCTGTCAGCGGCGCCCGCGCCCGTCACTTC
TGAGGTGGGAGGTGGCGTGTGTCCTGGAAACAGAGCAGTTCATTAGATGGTGAAATTAGATT
CCAGGTTGCAGCGTGGCGGCTGCCTAATGGACGAGGCGAGCGGGGCCCCCAGGGAGGCTCG
AGCAGTCCTGGACCTGGTGCCCACTCCGGCCTCACGCTGGCTTTCTGCTCATCCCCGGGGCCC
TGCTGGGCTCCCAGGTCCCCAGCCCCGGGGAGGTGCAGCCTCATCAGCATGGACATTGTTCT
GGACAGGGAATTTCTGCCTAAGGAGGCTGCGTGGGACCCCCCAGGACCCCCTGCAGTGGGA
GAGGGGTGCATGGTGGGGGCCGTGCAGCCAGCCAGCTCTGGCCAATCCGCACGTGGGCCTCT
GAGAGTCTCCGGAGCCTCTGTTTTCCCGACTGTGAAATGGGCACGTGACTGTAGGGCTGCTT
CCCTCCTGGCAGGTCATGGGTGTGCTGAGAAGCTTAGCCCGTGGAGTGGGAGTGGGGTCTTG
ATGTCAGGGGCCCCTCCTGACCCAACCCTGGCTCTGTCTGAGGCAGGGGCTGAGGGTGGCGG
TGAGGTGGTGACGCCTTCCCTCCTGCCCGCCCATCCTTCACGTGTTCCGTTTTCATTTAACTAA
ATACGCAAGATGAGATGAACAGCCCTGGCCAACTTGGGTCAGCCTCTGGGGAAGGGGCCTG
ACTGGAGGATCCAGAGTCGCAGAGCGAGGGGGCTGGCTGTGGGACTGACTCGGGGGGCCCA
GACCCCTCCTGCGTCGAGTCCTCTTCCCCAAGTGCATGAAGCGAGTGAGGAGCGGGGTGGCG
GCGTCTGTGTCATGCTTTCCCAGAAAAGAAATATCCCCTTCAACCAGAATGGGGAAGGAAGG
CGGGGGAGACCTGGAAGCACAGGAGTCTCCCGGAGGTGGGGGGCCCATGAGCAGAGGGGTC
TCCCGGAGGTGGGGGGCCTGTGGGCAGAGGGGCCTCCCGGAGGTGGGGGGCCCGTGGGCAG
AGGGGCCTCCCGGAGGTGGGGGGCCCGTGAAGCAGAGGGGCCTCCCGGAGGTGGGGGGCCC
GTGAGCAGAGGGGCCTCCCGGAGGTGGGGGGCCCGTGAAGCAGAGGGGCCTCCCGGAGGTG
GGGGGCCCGTGAGCAGAGGGGCCTCCCGGAGGTGGGGGGCCCGTGAGCAGAGGGGCCTCCC
GGAGGTGGGGGGCCCGTGAGCACAGGGGTCTCCCGGAGGTGGGGGGCCCGTGAGCAGAGGG
GTCTCCCGGAGGTGGGGGGCCCGTGAGCAGAGGGGCCTCCCGGAGGTGGGGGGTCCGTGAG
CACAGGGGTCTCCCGGAGGTGGGGGCCCCGTGAGTAGAGGAGTTTTTGCATCTCAAGGCCTG
AGGGTGCCAGCGTGGGGGCCCCAGGACTGTGGTCCGGCCTTAACTGGGCGGTGGGGGCCCC
AGGACTGTGGTCCGGCCTTAACTGGGCACCCCTCTGGCCTGGTGACCCTTTGGTCCCTCCCAT
GGGCCCCTCCCTGCCCCAACTTCTCCTCTTTGCACTTGGGTCCCAGGTCCCAGGAGGAGATCC
CCCGCTGCTCCATGCTCAGTCTGTGACTGCATTCGGGGTGCCTGATCCCCGCCATGGCCTCTC
TTGTGATCTGGGGCTGTCTGATGGGCACGGGGCTGGGCTGTGCGTCAGGGACGTGCGGTGAG
CCCCAGCCGACGCCCGCGGTCCAGGTGTCCCCCTGCCCCTCTGCTCAGTGGTGGGTGCCAGG
GCTCTGTGGATCCTCCCTGCCCCCACCTTGACTCTTTGCCTGAATCTGGGGCTGGGGACCAGC
TACTTGCCTTATCTCCTTGGCAACGGCTGCCAGGGAAGCTCCGGAAGTTGAACCCATTATCAT
AATTAGCTTCGAAACCCTTTGTGTTGTAATTCTGGGCTGCAGAGAAGCTGCTCGGTCCTCCCA
TCGTCCTCCCAGCGTCCTCCCACCGTCCTCCCATCCTCCCATCGTCTTCCCGCCGTCCTCCCCT
CATCCTCCCCCATTCTCCCGTAGGAAGTCTCTTCTTTCTTTTCCTGCAGACTTGGCCGCTGCCT
GGGCAGGAGGGGGGGGCAGCCCTTGCCGAGCCAGCGAGACCTCAGCTGAGTGCCCCGTGG
TGGGCTGTGTCCCCCAGCTTAGTGCAGGAGGGGCTGTACCCACACTGTGGGCCTGGGTCTGA
GGTGGTGATGGCCCCGTCCCGAGAGGCCTCTGGCAGCTGGCCCTCAGGGAGTGTCCCACATT
TGGACGCTCGTCGTGCCTGGGACCCTGAGGCCACATGGGCAGGGAGTGTGTCCCAGTGTTCC
CGGGGAGCCTCCTCTCCCGAGGCCTGGGGGATGCCATCAGGGGTCTCCTGCCGCCCTCTCCC
TCCCCTGGTTCAGAGGCAGAGGCCCTCACCTGCCTCAGTGAGCCTCAGTTTCCCCTTCTCTAA
GATGGGCATGAGTGTGGATGTCTTGGGGACACCACGGTTTGTAGGCGGAGGCCTGGAAGCCC
CTGTGTCTTCCCCACGGTTCGGCCGACCGGATGGCTGGCCAGTGCGTTTGGGAACTATCCGC
AGTTTGGAGGTTTCTTCCCTGGTCTGTCCTGGGGCCTGGAGCCACGGCCGAGGCACCGGGAT
CTTTGCGTCCCCCAGACGGGCCCAGACGCACCCCGTGCCGCCTCCTCCCTCCTCCAAGGCTCT
TCTCGCACGTCCGTCTCTCTGCCTCCCCTGCACCGGCCGCCCCCGGCAGCGCCATCTGTGCCA
TCTGTTCCAAAAACTTAAAAAACAAAAGTCGGAGCTGTTGCCTGCACTTTCCGGGTGGGGCT
GTGGCAGGAGGGGCAGCGGCGGGAGGGGCAGCTGCTTCCTTCATGCAGGGTCTGCGGGCCG
GCGAGGCGGGGGCAGATGGGCGGAAGCTGAGCTGGAAGGAGCTGGAGCCTTCTGAGGGCCT
GGCTGCGAGATTCATCTCCCTGAATTCTGGCTTTATTCCTCCTGGTCCACGCCTGCTGGGAGG
AGCCACAGATCTCGGCCGCATCCGGCTGCATTCTGTCTCCAGGGTTTCTGGAATGCTCTGAGA
TGCCTGGTCGGAGCCACCTTAGGAAGCAGGCTGAGTCCTGAACATTTGGGACCTGGGCCTCG
TTTTTTTTAGTTTAGAGGAAGGTTCCCTGCAGCCCGAGGAGCTGCCTTCCTGGATGCGGCACT
GAGGACTCAGAATTCAAGTCTGTGGGGCTGTCGTTGTCCCCACCCCGCCCTGTTCTCAAGTCT
GGTCAGCCTCATCCTCGGTGGGATGCAGATACGCCTGCCCTTCTCTCGCCAGGCCTCCCCAGC
GGCTCCTCCCTCTGCTGGGAATGCCCTTCCCCTTCTCCCCCTGTGGCCAACTCCTCTGTATCCT
GTAGCCCCCCCGCCCCTGTACACGCCTCCTCCGGGAAGCCCACAGGATTATGTGCGTGGCTC
TGCTGGGCTGAGCTGGGTCCCTGAGCCCCTCCTGTGATGTGTGATGCCCTCTGTGTCCCCGCG
GCGCCCCTCCTGCCTCCCAAGCCTCTGGGCTCCTGACTGGGAAGCCAGGGACCCCCGCGTCC
TCCCCTGCTCCTACCCCGCTGAGAGTTCAGAGAAAACCCCGTTAGGCAGACAGAGGATCGCC
CGCAGAAGGCGCACGAGATGTCTGCGCATTTTTCCCAGGGCTCAGTTAATTGAAGAACTTGG
CCAACGAGCACAGGAAATTAATCGCGGCTTGGAAATGGGACGTCCCTTGCGTATGGTTTGTC
TGCCTGGGCCTCGGCACCACTGCCAGCGCCCCTGCCTGGAGCCGGGAGCTTCGGGGTCCTGG
CGGAGGCCTCTGGGTCGCACACGTGGGCGGGGCCGAGTGCCCCCTGGCGACGGTGCCTCAG
GGCCAGTGCCTTCGAGCTGGAAGCCGTGGGCCTGTGGCGGCGGGCGGAGGACTGGTGTCTCC
TGGGGTGCCCGACACAGGGCCTTCCTGGTGCTGCTGTGCCGACGTCCACCGCGGGGCGGCAG
CTGGATAGGGCAGGACCTGGCTGTGGCTTGTGGTCTTGGGGGTGGCGGGGAGGGGTGCAGC
GTGGGGCAGGCTCTGGCTCTTGGACCCACCCGGCCTCCACACGCGGTGCTGCGCTTTGAGCT
TCTCACTAGGCCCTGGACCCTCCCCTCAGCCTGCAGGGACTATTCTTCCTGGAAGCCCCCGCC
CCAACCCCTGCCCCTGCAGCTGGAGCTCCGGCTGGCGCGTCGTCCCGGGCTGAGGCATTCTG
GCTGGCCTGTGTGGAGGCTCCCAGTTCCCACGAGGAGGGTCCCGGTGGGGCCTCTCTCTGTG
GCTGCAGGCCAGCTGGTGGGGTCCTGGGCAGGTGTGTGGCCTGGACCTGCCCTGGAGCCGAG
CTGCCGGCGAGAGACCTGGCTCTCCCCACCACTGTCCTTCAGCATCAGTCAGGGTGTTCTGTG
CCTGCACCGCTGAATCAGCCCGTTCCTGCTGCAGTCCCAGGCCTCTGGCCGAGGCCCCTGTGT
CCTCTCCCAGCCCGTGATGCCCACTGTATGCATACACTCTGTATACACGCAGCTGCGCCATGG
TGCCCGGCTGGTGGCCACGCACCTGCCTCGACGCTGCTGTGCAGGTGTTTTTCGGCATGAAC
ACTTAGCTCAGCGGACTTCAAGTGAAGCTAGTGACCCTCTGCCGTGGGGGTGGGCGTCGTCC
CAGCAGCTGAGGCCTTGAGAGCAAAGACTGAGGTTTCCGGAAGAAAAAGGATTTTCCTCAA
GACCGCAACATGGAAGTCGTGCTGAGTTTCCAGCCTGCTGCCCTCAGCTCAGAGGCAGGCAC
TGGATTACAGCTTCTCAATGGCATGGAGCTGGGACGTCCTCGGGGTGTACGTGGGTAGAATC
TGAACTTGGGCTCACGCTAGAGATGCTGACAGTTTTCACATGAGACGCTTTAAAATGTGGCT
TTGGCCCCTGCGCCTTCATCTTTGGTTTGGGTGCTGGGCCAGAGCCTTCACAGGCACAGTTTC
TGCCGCCGCTGCCTGTGGCTGAGCAGTGCCAGGCCTCAGTTGGCTCTGGTGCTGGAATGGGC
AGTTGGTGGGGCTGACCTTGGCCGGGCCGCCTGTGCCCGAAGCCTCCACCTCTGCTTCAGAC
TGTGTGGTCCCGGGTGCTTCTGAAGCACGGCCGTGGTCCTTCCCGGGGCAGATCTTGGGAGG
ACCACCCTGTGCCCCGCCTTCCCTGCCCTGGACGCCCTGGGCCGTGCAGCAGAGGCCGCTGC
TGGGGAGTCGGAAGGTGGAGCTATGGGGACTGCGCCTGCCCAGGCCAGAGGACTCAGGCTG
CCTGGACTTGGGGGTCTCCAGCGAGTGGGAGGGGGGCTGCAGGGGGCCATGCTGGCCTCCCC
ACATCCAGAGGGTGTTCAGTGGGGAGATGACTGGGTCCGGGCAGAGGGAAGCCGTGGGGGT
GTCCGTCGTGAATCGAGCCTGGAACAGCATGGCCACCCTCTTGTTCTTGGCTGAAGGGAGCT
TGCCCAAGTCCCCAGGTCCTGGAGGCACTTTGCACACAGGATGGGCTTGACACGAGGTCCAC
GCGGGTTTGTGTCTTTCCACAGGGTCAGGCTTTTATTGATGCTGTTTCAATCACAAAAGCCAC
GAGCTACGTGGAGTCCCCAAGCAGGCAGGTTCTCCCTAGAGCTTCTGGGTCATGCTGCGGTC
ACGGCCACTCCATGTTGGTCAGTGTTGCAGACCGTATATTGCCGTGTGCTCATCTGGTCAAAT
TCCACTTTCCACCCAAAGCCAGGTTTAACATCATGAGGAAAAAGACACAAGACAGAGGGTT
AATACCAGCCCAGAGGCCTGGCCTGGGCTGCTGCAGGTGACTGTGAGACAGCGGCGGTGAA
GACAGAGGCCTCCAGGGAGAACTGAGCACGGAGGACTGGCTTGTGTGTGTCCTTATCTGGTT
GGACTTGGTCTTCATTTTTTTGTTTGTTTGTTTCTTTTTTTTTTTTTTTTGGGACAGAGTCTTGC
TCTATTGCCTAGGCTGGAGTGCAGTGGCATGGTCTCGGCTCACTGCAGCCTCCGCCTCCTGGG
TTCAAGCGATTCTTCTGCCTCAGCCTTCCAAGTAGCTGGGATTACAGACCTGCGCCACCATGC
CTGGCTAATTTTTGTATTTTTAGTAGAGACAGAGTTTCACCATATTGGCCAGGCTGGTTTCGA
ACTCCTGATCTCAAGTGATCTGCCCGCCTCGGCCTCCTCCCGAAGTGCTGATGCGTGAGCCGC
CATGCATGGCCAGGTTTTCTTCTTTTTTGCTTTTTGGTTTTTAAACAGACATCTTATCCTTCTTG
GTAGAGTGCCCTGTGAGACAAAATGGAGTCTTTTCCTAAGATGGAGTTAGTTATGCCAAGGG
CTCTCTGCACAGGTCTGCCCTCCCCTGCTTCCAAGCCCCCGCAGCCTCGGCTCCACAGACGCT
GCTCTGCCAGCTCTCACCTGGGCGCCTGCCACTCTTGTTCAACCTTGACCCCACCTTGACCCT
GTGTGACCTCTGCGCTAGGGATCTCCTGGCTTCCCCCACCCCCAGCAGGTCTTCCCCCACCCC
CAGCAGTCTTCCCCCACCCCCCAGCAGGTCTTCTGGGGCTCTGCCCCCAGGGGTGACTGCAG
CGCCCAGTGCTGCCTCAGGGAGTCCTGGCCCTTGCATGGGGCAGCCACAGCGTCCCACACCT
GGGGCAGCCCCAGGGCCCTGGGAGCGGGGGCAGGCATCAGTGCTGCCTTATTCCTGGAAGC
CTAAAGCCACTTGGGGAGGGAGATCCAGCCCTGACCTCATGTGTGGTGGCCTTGCTGGGCCT
CTGCGGCCGCCCTCGTCCGGCACGTGTGCTGGGCCGTGACCTCTGGAAGAGCAGCCAGGGTT
TCGTCTAGGTCCCTCCGGAGCAGTGGGAAGAGGGTTTGGGGCACTTCGTCCCTTTGGGTGAG
GGAGGGGTGGGGGAGGAGTGGTGGTGGAGGTGAGGGGCCTGTAAGTGAGGGACGGGGTGA
GGCGGGGTGAGGGGCCTGTAAGTGAGGGAGGGGGTGAGGCGGGGTGAGGGGCCTGCAGGG
GCAGAGGCAGTGAGCTCGTGTGGTGGCGGGCGGGTTCCAGGGGCGCAGGCAGCGCAGGGTT
GTGTGCTGGGTGTGGGTGCTGGTGATGGCACTGATGGTGCGGGACGGGGTCTGCTCTGAGTG
CTGTGTGTCAGATGTGGGCCCAATTACGTGCTCCAATTGCTGCCACCACCCAGTGGGCTGGTC
CTGGTTTGGGGGCTCAGAGCCTGAGCCTGGGGTGATGGGGGCCCCGGGTTTGTGCCCGGATG
GCCTGGCCCTGTCCTTCTGCCTCCGCGGCCAAAAGTCTGAGAAGTCTCCCTGCAGGCCGCAG
ACACTGGGGCTTCCCTCTTCCCTCTTCTCCCAGGAGCTCAGACGCTGGGGCTTTTCCCTCTTCT
CCCGGGCTGTGGAGCTCGGCCTTTGGTGAGTCCCTCCTCTCTCTGCGGTGCTCCCCAGGGGCT
CTCCCACAAGACAGTGAAGCCGTGAGCAGAGTGGGGTCCTCTTCCCACCAGGCACCACAGCC
AATGCTGACCTCTGCTCCCCAAACCCTCCAGACCCTCCAGGCACTGCGGCCAACGCTGACCT
GTGCTCACTAAATAGCTCCGCACATCGAGGACTTGTTTTTTCCTTTTTGATCCGTGGCAGCTT
GTGTCACAGCCGCGTGGCTTGTCTGCCCAGGGAGGAGCTGTGGGTCAGAGTAGCATGTGGCC
ACAGCATGGTGGAGTCAGGCGTGGGCTCGCAGCAGGGACACACCGATGTCAGCTGCTCATA
GCGTGAAAGGTCATGGTCTTTTATTTGCAAGGGGCAGAAAACAAATGCAAAGGGTTGTAAG
GAATGGGCTTACATAACCTGGAAGTCCAAGGGGCTTCAGGCACAGCTGGATCCAGGTGCTCA
CATTATGTGTCTTAAGGCCTCTTCTCCCATCCTTCTGTGCTGCCTTCCTCATGACCTCATCTTC
AGGCAGGTTTTCCTGTGCCCACCCGTCCTCCTGCCAGTGCCCCAGGCCTGCCTCCTCCTAGTT
GGCAACCTTTGTGGGAAGGGGGTGTCTTATTCCTGGTAGTTCCAGCAGAGATTCTGGGGCCA
CCACTCGCTGGCCGTGGTTGGCCAGCCTCGATCCCATGAGCATCCCTGCACCCCCAGACTGG
AAAGGAAATTGCAAGGCTGAGACAGGAGGGGTGGGTCTAGACAGGTGCTGTGATGCTCCGA
AGCTCCAATGATGAGGGGGCGTGACCAGTCTGGAGAGACTCCCCCCCAGGATGCCCATGCCT
GTGGCCAGTGCCCCGGCCCAGCAGGTCAGGGGTGGGCGTATCCGGGCTGGAGGGACTTCCC
CCAGGATGCCCGTGCCTGTGCCCAGTCTCGGCCCTGCAGGTCGGGGCATCCAGGGAATATTT
CCTCTGTGGGAGTGGGGAGAGGGGAGCGGGGACACTGCCCGGAACCCAGGCTGGACTGCAG
CACTGCTTTGGGGACAGAGGGAAGGAGATCCTGAGTGTGGGGAATCTCCCTCCCTTGACACC
CACCTGGATGTGGGCGCCGGGGAAGGGCTCGGGTTTGAGACACAGGAGGGCCCCACAGGCT
CCGTGGCGGTCGTGTGGTGCCGCAGGGTGTGCAAGGTGATGGCGAGTGTGAAGACAGTACA
GGGGCGCGGACACGGGGAAGCGGCTGCTCTGTGGCTCCGTCCCCGCCGGCTCCTGGTTCTGA
GCACGGTTGGAACGAGAGGCAGCAACACGGTCAAGGTGAAGGCGTCTCTCACATTGCCTGG
AATGAGGGCGTGTCCCACGCATGGCCTCCTTTATGGCTGTGACGAAGGACCTGGACGTTGGA
CACGTCCCCTTCACATCCCAGAACACCTGCGAAAAATGTAAATGAGACAAAGCATTTGCTTC
CTGCGAAGTGGGTTTCTCTCTGGTGTGCATTCAGTGACTTCAGCACGCGGGAGTTGGCTTTCC
TGCCAAAGCGGCAGCTTGAGGCTCTGGGTTGCACAGCTTTGCTCCCAGGAAGAGCTGGGAGA
TCTTCTATGTCTCTTCTATGTGGGTGGCCTTGTTGCAGCCACAAGCCGGGTTCCCGCGGCAGG
GTCTTCGGGGGCTAGCGGGCTCCTCCTGTCTGCAGCTTCCTGGGGTGGGCAAGGGAGCAAGT
GTAGCAGAGTCGCTTGTATCAGAGGAGAATGCAGGGCTGAGAAAAAGTCACGCATTTGGGC
CGAGCATGGTAGCTCATGACTGTAATCCCAGCCCTTTGGGGGGCCGAGGCGGGAGCATCACT
TGAGCCCAGGTGTTCGAGACTAGCCTGGCCAATGTGATGAAACCCCATCTCCACTAAAAAAA
TACAAAAATTAGCCTGGTGTGGTGGCACATGCCTGTAATCCCAGCAACTCTGGAGGCTGAGG
CGGGAGAATTGCTTGAACCCGGGAGGCGGAGGTTGCAGCGAGCCAAGATCACGCCATTGCA
CTCCAGCCTGCGCGACAAGTGAAACTGTATCTCAAAACAAAACAAAACAAACAAAAACAAA
AATACCCACATGCATTTGGGGGAATTTTGGTGGGGTGTGAACTTGAGCATCTGAGGGGAAAT
TGAAGAAGGATCAGTGAGTGAGGTAATCGCGTGGGACGTGAATGGGAAGAGCAATCATGGT
GGAAGTCACTGGCGGGAGGAGGTGCCTGCAACCCGGGAGGAAGGAGAAACGGAGCCTCCTG
GCAGCCAGGACGAGGCTGGAAAGAAGGTGATCCGAGGAGCTCCTGTTGCTTGGCTCCGGGTT
TAACTGGCAGAGAGAAGCCCTCTGCGTTGGGGTCTGGAGGAGGTGGCCGAGAGTGAGGAGA
GGGGAAGGCTGATCGGTGCCTTCTGCCCCAGGGGTTTTGCTTGTTCCTGTGTTTGTAGCAGTT
GATGGGCAGGCCTTGCTGTTCCCATTTGACTGAGAGTTTTGGAGACGTTGCCAAAGGATGAG
GCTGCCAGGCTGAAACCAGACAGAAGGGGCAGGGGCCAGAGTGGAGAGGGGGGTGCGTCCT
GGAGGGGGCAGCTGCGCATAGAGGGGGCAGCTGCCACTGTGGAGTTCAGGGTCCCGGGAGG
TGGCTATGGGGGTGCCCTCTGGCCCGAGCGAGCTCTGGTGGGGTGGGTGGGGAGTGGGGTCC
TGCGTTCTGGAGACGCGGCCATGCTGGTGCCCTGCTGCGGCCTGGTGGGGAGTGGGGTCCTG
AGTTCTGGAGACACAGCCATGCTGCTGCCCTGCTGCGGCCTGTCTGAGAGCGCGCGTTCCCT
GCCCCCAGGTTCCTCCTGGTTTTCTCCTGCCTCGTGCTGTCTGTGTTTTCCACCATCAAGGAGT
ATGAGAAGAGCTCGGAGGGGGCCCTCTACATCCTGGTGAGCCCCGAGGGAGGGCGGGGGCT
GGAAGTGCCCAGGAAGGAGCTGGAGCTGCCTGGGCGTCTGTCTTCCGTTCCTGTTACTACAG
GGCCTCTGACGCCCCCAGCCCCAGCTCTGGCCATGTCTTTGTGCCCTGTCAGTTCCCATCTGC
CCCCAGCCCCACAGCCCGGGCTATGCCCCAGCCCCACCCTGGCTGTAACTCAGCCCTCACCT
CACCCCTCCTGGTGACCTCACAGTGGCTGACAGCCCCGCCTCCATCGCCCCCACCCTGGGTG
GGCCACCCTCCCTTTCCCATCCCCACTTTATCTCTGGAGCCGGAGATGTGGGGCGTCCCCAGC
AGACGGCAGACGTGGGCGTCGGGGTGGGGGCTGTGACGCTTGCAGCAACCCTGTGCCAGGG
CAGGGTGGGCGCCATGGCAACGGGAGTGCAGTTCCTGTGTTGCTGTTGGTAACGGGGTGGGG
GGCCCTCAAGGAGACTGGTCCCCATCCAAATGCACCTCCCTCTACTGCCCGCCCTGCTCCTCT
GTTCTGGGGCTTCCCAAGGTCTGGGTGGAGACTGTGGCCCCCTTTGCAGTTACCTGGGGCCTT
CCCTCAGCTCAGCCCTGCTGTGCACCCCCCAACTCAAGGCAGGTCCCCCACTGGCTCAGACA
GGGTCCCCCAGCTCAGACAGGGGCCCCCAACTCAGATAGAGTCCCCCAGCTTGGACAGGGC
AGCTCCCCAACCAGCTCAGACAGGGTCCCCCAGCTCAGACAGGGTCCCCCAGCTCAGACAG
GGTCCCCCAGCTCAGACAGGGCCTCCCAGCTCAGACAGGGTCCCCCAGCTCAGACAGGGTCC
CCCAGCTCAGACAGGGCCTCCCAGCTCAGACAGGGTCCCCCAGCTCAGACAGGGTCCCCCAG
CTCAGACAGGGCCTCCCAGCTCAGACAGGGTCCCCCAGCTCAGACAGGGCCTCCCAGCTCAG
ACAGGGTCCCCCAGCTCAGACAGGGTCCCCCAGCTCAGACAGGGCCTCCCAGCTCAGACAG
GGCCTCCCAGCTCAGACAGGGTTCCCTAGCCCAGATAGAACCCCCTAGCTCAGACAGGGCAG
CCCTGCCAGCTCAGACATGGTCCCCTAGCCCAGATAGAGTCCCCAGCTCAGACAGGGAGGTG
GGGAGGGTGGCCCCATGGTGTTCATGCTCAGCTGTGTTCCCGGATGCAGGGTCTCCAGAGGG
CCCAACCCTTCCTGCCCAGAGGCTGGAGAGGGGTGGGGGGGTCAGCTTGCAGTTTCCTTTGG
GGGCCTCCTGCCCTGTGGCTTGGTCCCTGGGCCAGAACTGCTCCTGTGGGTGTCCAGGCTGG
GCCCCCAGGCCCTCCCCCAGGCCTCAAGGTGGCCTCAGCTTTCCTCCCCTGCAGGAAATCGT
GACTATCGTGGTGTTTGGCGTGGAGTACTTCGTGCGGATCTGGGCCGCAGGCTGCTGCTGCC
GGTACCGTGGCTGGAGGGGGCGGCTCAAGTTTGCCCGGAAACCGTTCTGTGTGATTGGTGAG
GCCTGGTGGGGGTGGTATTGCTAGAATCAGGGCCAGGCACCCAGGGACGGACTCAGCCCTG
GGGGGAGCTGGGGCGTCTGCGTGGGCCAGAGAGGCTGGGCAGGACTGGCTCCTTCTGGAAG
TTTCTTTTCACCTGCTGACTTGGAGTCAGAGGCTGTGGTTAACTCTGCCTAAATGTCAGGAAG
AGGAATGTGGCGCTGGGCTGCCCATCCTGGGCCCCACAGGCAGGGTGGACGATAGTAATGTC
CTTCCTGGGGCCTGACAGAGCCCACACCAGGCGCTGGGCATACACCTTCCCGCCCCTCTGCA
CATCCTCCTGGAACGCGGGGTGGGGAGTTTTCTCCAAGGGGGTGCGGGGAGCGGCCCAGCA
GCTCACCAGCTCCACGCCCGCTTTGTAGACATCATGGTGCTCATCGCCTCCATTGCGGTGCTG
GCCGCCGGCTCCCAGGGCAACGTCTTTGCCACATCTGCGCTCCGGAGCCTGCGCTTCCTGCA
GATTCTGCGGATGATCCGCATGGACCGGCGGGGAGGCACCTGGAAGCTGCTGGGCTCTGTGG
TCTATGCCCACAGCAAGGTGAGTCACGGCCCCAAGGCTGGCGGTGGGCGCCCCCAGCCAGC
GAGAGTCCTGGCCCAGACCGGGCCCCACCCCTGCCTGGGGTTTGCTTCAAGAGCCCTGGGTG
GAGGGATGGAGTCAGTGGTGGCTCTGGCTGGAGCCCATCAGGTGTGAACGAGCCTCCCTCCC
CTTTCTTCTCGGTGCTTCTTCTCGTGACTTGGGCCATCTTTGTCATCTGTCTCCAGAAAGCTGC
TTTTTCGAAAGGCCAGGCCAGGCCATTTTGGCCCCTGCCACCCCCACCTTCCTGCCGCGAACC
CTTGGCTCTGTGGTTGGGTGTCTTCTTTCCTGGGTTTTTAGTGCCTGGATCAATACAAAGTGCT
AGTCACCTGCCTGGTGGTTGTTTAAGCTGCGGCTTGCGGCGGCCGCTGTCCGGGGGACCCAG
AGGGTGGCTGTGGCCACACATGCTGAGTTCCAGGGGCCCCAGGCCCCCCAGTGACCGAGAC
CCCATCCTGCCAGACTGTGCTGTCCCTCCACCTTGCTCACGGGTGGGCTCATTTGCTCAAATG
GCCCCCTCCCCATTTTCTTCCCTCTTGAGCCCTGGCAGGGTGGTGGCTTTGTCTGCAGAATGG
GCAGGGGTCAGCCCCCGGTGGCCCCTCTCCCTCCCTGGACGCTGTGGGGGTTGAGTGCTGAC
TGCAGTGGGCACCCTTTTCACATCTGTGCCCGGAGGCTCAGGGAGGTTGGGGGAGGGTCTCA
ACAGTCCCAAGATTCTCCAGCCCTGCTGGCCGCCAGAATAGACGCTACTGACCCTGGAGGGC
ACGGTGGGCTGGCCGTGCGAGCCACTGTTGCCCAGGCCAGGAGGCTCAGGGGAATGGGCTG
CCGTAGCTAAGATGGTGAAGCCAGCGGCGCCTCCTGGTTCCCCTGTGCCAGCCCTGCCACCG
ACCCTGGCGTGGAGCCTCTGCTGGCCTCAGGAGACGGGTCCATCCAGCCCCTATATCCCTGG
AGACAGAGCCCAAAGTGGGCTGACTGGCGAGGCTGCAGTGCTCCTGGAGGCAGGTGGCGGG
TGGTGATGGTGGTGAGGGTGATGGTGGTGATGGTGGTAGTGATGGTGGTGGTGGTGGTGATG
GTCATGATGGTCATGGTGGTGGTGGTGGTGATGGTGATGGTGGTGATCGTGGTGGTGGTGAT
GGTGATGGTGGTGGTGATGATGGTGGTGATGGTGGTGATGGTGATGGTGGTGGTGATGGTGA
TGGTGATGATGGTGGTGATGGTGATGGTGGTGATGGTGATGGTGGTGGTGATGATGGTGGTG
ATGATGGTGATGGTGGTGGTGGTGGTGATGGTGGTGATGGTGATGGTGGTGGTGGTGATGAT
GGTGATGGTGGTGGTGATGATGGTGATGGTGATGGTGGTGATGATGGTGGCGATGGTGATGT
GATGGTGGTGATGGTGATGGTGGTGATCATGGTGGTGGTGGTGATGGTGGTGGTGGTGGTGA
TGGTGATGGTGATGTGATGGTGGTGGCGCCGGTGATGGTGGTGGTGATGATGGTGGTGGTGG
TGATAGTGGTGGTGGTGATGGTGATGGTGGTGATAATGGTGGTGGTGGTGATAATGGTGATG
GTGATGCTGATGATGGTGATGGTGGTGGTGATGTGATGGTGGTGGTGATGGTGATGGTGGTG
ATGGTGATGGTAATGGTGGTGATGGTGGTGGTGGTGGTGATGGTGATGATGGTGATGGTGGT
GGTGATGGTGATGGTGGTGATGGTGATGGTGGTGGTGATGGTGGTGATGGTGGTGGTGATAA
TGGTGATGGTGATGGTGGTGGTGATGGTGATGGTGGTGGTGGTGATGGTGATGGTGATGGTG
GTGGTAATGGTGGTGGTAATGGTGATGGTGGTGGTGGTGATGGTGATGGTGGTGATGGTGAT
GGTGGTGATGGTGGAGGTGGTGATGATGGTGGTGGTGGTGGTGATGGTGATGGTGGTGATGG
TGATGGTGGTGGTGGTGGTGGTGGTGATGGTGGTGGTGATGGTTTTGGTGGTGGTGATGGTG
GTCATGGTGTTGATGTGATGGTGGTGGTGTTGGTGGTGGTGATGGTGATGCTTCTGGTGATGG
TGGTCATGATGGTGGTGCCGTCTGCCTCTCAGGAGCTGGTCACTGCCTGGTACATCGGCTTCC
TTTGTCTCATCCTGGCCTCGTTCCTGGTGTACTTGGCAGAGAAGGGGGAGAACGACCACTTTG
ACACCTACGCGGATGCACTCTGGTGGGGCCTGGTGAGTTGTGGTCATTGTGGTTTTCCCTTTC
CCTGCTGATACACCCCTGTCCCTGTGCTGGGACCAGGCTCTCACTGGCTGAGCCTGCTCCATA
CATCTCTTTGGGGCCACCTGCTGGCTGCCCGTGGTCATGATGGCTTGTGGTCGAGGCGGGGT
GGTGGTTGCCAAGCTGGCGGGGGAGTGGGTGGGGAGGGCTGGGCCCCAGAGAGCCTGAGCT
CAGCCGTGGTGGGCATCTTCCTTCCTCCTGAGCTCAGCCGTGGTGGGCATCTTCCTTCCTCCT
GAGTGACCCTCCACCTGCTCCGTGTCCCGATTATTGGGGGAAAGGGGAGAAGAGGCATCCCT
AGGAGATCTGTCCTGGGCAGCCCTGAGTTTTTGGAGACTAGGTGGCTCTGCAGGCCCCACTC
TGATCAGGTCTGAACAGATCTGGCCCCTGGGCTCCTCCTCACCTGGGGCCACAGACACCCTC
AGGCTGCTGGTGCACAGGGGCAGGGTCTGTGCTCTCCTGGGAGGCTGAGACCAGACCCCGG
GCCTAGCCCCGCACATGCCGGCTGAGTGGGCTCAGCCCTGCGGGGAAGGCATGGGCAGCTC
CACCTGGTGCCTAAGCCGGATGCAGCTGATCCGAAGTTATTTTATTAATTTCATGAGACCTGG
ACACTGTCTTCCCAGTCGGGGCTTGGGTGGGGGTTGAGAGCTGAGCGTGCCCCGGGGCCCAG
GCTGTGGACCATACAGATGACCTGGACTCACTGAGCCATCTTGTGTGGTTCAGGCCCCACCA
CAGGGTCAGGGTGGGGCCCGGGGCGTCCTGAGCTGTGACTGCTGGGCCCTAGGACTTTTGAT
GGGGACAAGCCCATCCCCCAGGTCTTTTGTGGGCAACAGCCTTCTTATCTTTACGATTGGGTG
TTTTTTCAGAGTAATGCTTGTGTATATTTTTAAATGTCAGAGAGTTACTAAGCCTTATGGTGA
GAATAGAGCCCCACTCTGTGGCCGCGGTGCCCTCCCCTCTGGGTCCCTCTGCAGCGGATGCG
GCTGCTTCTGTGTTTTTCTTTGGGGTTTACCTGATTAGTCTTAAATAGCAAATAGGGATGCTG
GTATTTCCTGATTCAAACGCGCTGACTGTGTACCTCCTACTGGGATGAGATCTACCCCCAGTT
TCCCTTTCTTGCAGCCTTTGGGGGTTTTCAGCAGTGTGGGGGGAACCCCGCCCTCACCTCCCC
CGCAGGCCAGGCCCCATCTGCCAGGCCTCCGGCTTCCTTTTCCTTGAATACAAAAAATTAGC
CGGGTGTGGTGGCGGGCGCCTGTAGTCCCAGCTACTCGGGAGGCTGAGGCAGGAGAATGGC
GTGAACCTGGGAGGCGGAGCTTGCAGTGAGCCGGGATCACGCCACTGCACTCCAGCCTGGG
GGACAGAGCGAGACTCTGTCTCAAAACAAACAAACAAACAAACAAAAAACCACGCCTCCTT
TGGTAACGAAATTGTGTAGAGGGCTGAGGCCCTGGCTTAGTCTCCCCAGGAGAGGTTTCTTG
GTCAGGCCTCGCCCAGCCTCCCGCCCACTCTGTGTGGCAGCCTCCTTCCCAGAGTCTCCTGTG
CGCTCCGCGTGGTGGTGGCAGCCAAGGCCCCCAGGTGCTGGCGCTGCTTTGCACCAGGCCAC
AGCTTCCTGTCGGTGTGGAGCAGGTGCGGCCCAGCCGGCCCACCCTGTCCTCAGACCACACA
GCAGCGTCCTCGGAACCCAGGGTAAACTCCCCAAGGTGGGGCTGCAGGTCCTGGTGAGAGG
TCTCCCCTTCCCGGCCCTGCTGTCTGCGCTCAGGCCTCGATCCCTCTGTCTGTTTATCTAAGAC
TCTGCCCTCACAGGGCCTGTCCTGGGCCCACCTTCTGGTCCTAGCCAGAGTGGCCTCCGTGCG
GCTGTCCTGAGCCCGTGGCTTGTGGTGCGTGCGGTTCTGGCAGGCGGGGGTGGGCTGCGGCC
CCAACATGTCCAGCACTGAGTGCAGGGTGCCTGCCCCTGCCTTCCTCGGCCCCGGCACAGGC
ATCTCTTGTCGGAGCTGCTTGGAGACGTGTCCGTCCTTCGCTTCCCCTCATCCAGGCCCCGAG
TCGGGTGCTCCTGGGCTCACCGGGACCCTTGGGGAGGTCCAACCTGGCCTGACCTCCCCCTT
GCCCACCACCCCTAGAGAAACTAAGCACAACCCCTGGGGTCCCTGCTCCAGGAGCAGCCCAC
CCCGCCATGGCTCGGTGGAGACCACTCCCCCGCCTCTTGTGGTGGCCTCCTCCTGCCCACCCC
TCTCCTCTGGCCTCAGCTCAGCTCAGGCTCAGCGAGCCCCCTGCACGCCCTGCCCGTTCCTCT
GGGTCCCTCTGGGCACTCCCTTCTCCCCAGCTGCACTCTTCACCTGGCGGGGATGCAGTGGG
GGCTGCTGTCCTAGCCGAGCCTCGTGGGAGTCACGTCGCTGGGCCTCTGGTTCTCGATGTGA
GATGGGGACACAGCCTTTGGCTGGACCCCGCTTCTGGCCTGTAGGGGAAGAGGAGAGAGGG
CTCAGGGGATGAGGGGCGGCCGACACCGACCCTGGCCTCACTGGGCCTCCGTGTGGATGGTG
ACACGGGGGTGGCCCCGCATCTGTCCCATCCCAAGCCCCGAGTCGCCAGCGGGCGTCCAGCC
TGCCCTCAGGGGTGTGAGCAGGCCCTTCGTGTGACTAGAGCCTGCGGTCCCACAGATCACGC
TGACCACCATTGGCTACGGGGACAAGTACCCCCAGACCTGGAACGGCAGGCTCCTTGCGGCA
ACCTTCACCCTCATCGGTGTCTCCTTCTTCGCGCTGCCTGCAGTAAGTCCAGCTGCCCCTGCC
TGCCTTGGAGGGGGACGAGGTCTTGTAGGCTCCCGAGGTGACCACAGGCCCCTGGGCACAGT
TCCCTAGGTGGGACCTGGGGCAGGAGCAGCTCTCAGCAGGTCCACAGCCCCCAGGAGCTGG
AGGTGGCAGCTCAGGGTCAGAGGCCTTGTTCCCCAAGGACTGGAGTGGGGGTTCCCCAGCCC
CGACAGGAGCATGCCCAAGGCTGCGGCTGTAGCTTCAGGGGGCTCTGTTAGTCACTGGTGGC
CCCTCTTAGTCTGAGTGGGGCTGAGCAGGAGGTCCTTGTGACCAGGAGCAGGGCGGCTGGTG
ACACAGGTCCCTCATGGGTCTCTAGGCAGTGAGGCCCACCCAGCTCAGAGGGAGGTGGAGG
GGCCCTGTCAACCCTTGTTGCCCCAGGGCAGGGCAGGGCAGGGCAGCTGGTGCACGGCAAG
AGGCTGGCCCCGGTGTCCCCTTCACTGTGTGCCCTTCAAGATGGGCCTGCAGACTCTTCTGTG
TGGAAGGGAAAGAGGCCACTCTGAGTTCAGTGTGGGTCCTGCCATGTCTCCTGCAAGCCAGA
GGGTTCCTTCCTCTTGATGCTGACAAATTGTGGGGATATGGGTTCCTGGATTAGTCTGACCTT
GATGAAGGTGGGGGAGGCATGGATCGGTGGAGACGGGTCTGACCCTGATGAACTGGGGGGT
GTGTGAGTCCCTGGGGCGTGGTCTGACCCTGATGAATTGGGGGGTGTGTGAGTCTCTGGAGT
GTGGTCTGACCCTGATGAATTGGGGGGGGTGTGGGGCCCCAGAGCATGGTCTGACCCTGATG
AATTGGGGTGTGGGGGGTCCCTGGGGTGTGACCTGACCCTGATGAATTGCAGGGCATCTTGG
GGTCTGGGTTTGCCCTGAAGGTTCAGGAGCAGCACAGGCAGAAGCACTTTGAGAAGAGGCG
GAACCCGGCAGCAGGCCTGATCCAGGTGAGTCCAGGTGTCCCCCGGGGACCAGCACAGCCC
TTGTCCTGGTCCCACCTTGTTGAGGAGTGGAGGCCGCTGGGGCTTTGGCATTGCCCTGTCTGT
GGATGGGCCACAGCGCTCACCCTCTGCAGGAATCTGTGGAAGGAGCGAGGATGTGAAGTGT
GTGTGTTGAGAAATGGCTCCAACCCCTGAGGCTGCACAGGGGTGGCTCGTGTGAGGACGCTG
GGGTGGCCCTTGCCTTGTGTGGGTGCTGGGGCCCGGCCATCAGGTCCTCTCTACCGCACCCTG
GGAGGTGAGCACACCCGTTCTGCAGCTGCAGGGCCAAGGCTCCTAGAGCCCACACACTGCA
GTGAGTCAGTGCCTGACCTCGTCTGACCCCTGATGCTGGCTGTGGGCCCCCATGATGCCCGC
CCGGACCCCACCCAGTGCCGTCCCACCATTTGGGGGTGGGTAGCGTGCAGTGACCACTGTGG
CATCCACAAGTAATTCCTGAGCCACCTTGAAAATACGGACAGACAATGGCCGGGCACAGTG
GCTCACGCCTGCAATCCCAGCACTTTGGGAGGCCGAGGTGGGAGGATCACGAGGTCAGGAA
ATCAAGACCAGCCTGGCCAACATGGTGAAACCCTGTCTCTCCTAAAAATACAAAAATTAGCT
GGGCGTGGTGGCGTGCGTCTGTAATCCCAGCTACTCGGGAGGCTGAAGCAGGAGAATCGCTT
GAACCAGGGAGGCGGAGGTTGCAGTGAGCCGAGATCACGCCACAGCACTCCAGCCTGGGTG
ACAGAGCGAGACTCCGTCTCAAAAAAAAAGAGAAAATATGGACAGACAGCAGGACCCCCTT
GGGACCAGCCATTCTTGCAGCTCCTCCCCAAAGTCTTCCATCCACTGGACCCGCAGAGCTGA
CCAGGATCCCTGCCAGCGGAATGTGGCTATAGGCTCTCTCCTGTCTTAGACAGCAAATCTTG
AGATGGAGATAATCCTGGGTTACCCCAGGTGGGCCCTAAATGCAATCACATGCATCCTTATA
AGAAGGAGATTTGACACAGAGGAGGGGAAAGCAGTGTGACCACAAAGGCGGAGACTGGAG
ACGTGGCCACAAGCCGCGGAGTGTCAGTGGCCACTGCAGGGGATGTGCCTCTTGTGGTCTTG
GCTCTGGGTCCAGCCTACCTTAAGGTGTGTATTTATCTTCCACTATAAAAACTACAAACAGCT
ATTTATTTCCAGGCAAGAGTTTTAAGAAGAACCAAAATGCCCCCAAATCTCACTCCACTATT
GACGGTTTAAAAAGTGACCACGTACGTGTGGTGAAAAGGTTACAATAGTGCCAGAAAAAGC
AGCGTCTGAGCACCCAGGGTCCCATGCGCCCTGCTTCCCTCGAAGGTCACTCTTAGCAGCTG
GTGCACATTTCCAGAGCACAGCTGGGCCTCTCCACAGGCGCACCTCAGAGGACCTGCAGCCG
TGGTTCCAGACCACTACGGTAGAGATACGGCAATAAAGCGAGTCACACAAATTTCGTGGTTT
CCCAGTGCATATGGAAGTTATGTTTACAGGCTGGGCACCGTGGCTCCCACCTGTAATCCCAG
CACTTTAGGAGGCTGAGGCAGGCAGATCTCTTGAGGTCAGGAGTTCGAGACCAGCTTAGCCA
ACATGGTGAAACCCCATCTCTACTAAAAATACAAAAAAATTAGCTGGGCGTGGTGGCACACG
CCTGTAATCCCAGCTACTCAGGAGGCTGAGGCAGGAGAATCACTTGAACCCGGGAGACGGA
GGTTGCAGTGAGCCGAGATTGCGCCACTGCATTCCAGCCTGGGAAACAGCCCGAGACTCCGT
CTCAAAAAAAAAAAAAAAAAAAGTTATGTTTATACCATGCCATAGCTTACTAAGTGTGCAGT
AGCATTATGTCTAAAAAACAGTAAAACACTGCACGTACTTTCACTTACAAATACTTAATTGCT
AAAAAAATACTAACAATCACCTGAGCCTTCTGCGAGTTGCAATCTTTGTGCTGGTAGGGGGT
CTCACCTCGATGCTGGTGGCCACTGCTGATGAGGTGGGGCTGCTGAAGGCTGGGGCTGTGGC
GATTTTTTTTTTTTTTTTGAGACGGAGTCTCGCTCTGTTGCCCAGGCTGGAGTGCAGTGGTGC
GATCTCAGCTCACTGCAAGCTCCACCTCCCTGGTTCCTGCCATTCTCCTGCCTCAGCCTCCCA
GGTAGCTGGGACTACAGGCACCCACCACCACGCCTGGCTAATTTTTTGTATTTTTAGTAGAGA
CGGGGTTTCACCGTGTTAGCCAGGATGGTCTCGATCTCCTGACTTCGTGATCCTCCCGCCTTG
GCCTCCCAAAGTGCTGGGATTACAGGCGTGAGCCACTGCGCCCGGCTGTGGTGATTTCTTAA
AATAAGGCAAGAATGAGGTTTGCCGCATCGATTCACTCTTCCTTTCACGAAAGATTTCTCTGC
AGCATGCGATTCTGTTGGATAGCGTTTTACCCAGTGGACCTCCTTTCAGCATCGCAGTCAGTC
CTCTCAAATGCTGCCACTGCTGTATCAACTAAGTTTATATAATGTTCTCATTCCTTGTCATTTC
AACACAGTTCACAACATCTTTGCCTGGAGTAGATTCTGTCGACCAAAAAAAAAAAAAAAGA
AACCACACACATGTCAAATGTATTTGGGAATTAGAAGAAAGGATTATAACCAGAGATGAAC
CACTGTGGCCAGAGGGTGGGGGACTCTCGAGAGATGAGCCAGTGTGGCGAGCCACCCATGT
GCCGGAGAGGGAGGGGCAGGGAAGCTCTCCCTGGCTGAGAAGTTCACATAAGCTGCTTGGA
AACAGAGTTCACTGGCTCTGAGGCTGAAAACCAGAGTTGGCAGCCGTTCACTGGTTGAGATG
CCGCTGCTGGGCCAGTGTTCTTCCTAGAGCATCTTGTCTGAATTGCTGCATTCCTGATAAGGA
ATTCCTTGTGGGGTTATTTTAGAATGTCTTTGACACTGTCCTCATCTCAGCCATGCAGGCGTG
AGCCCTGCCGCCGTGTACTCTCTGGCCTCGGTTGGTTTGGACCTGACAGAAGTGACCTTACCC
TGCTGTCTGCAGTGTTCACCGTCCATCCCAAGAAACCACTTACTTTGCTCATCCATAAATGCT
GCTCCTCACCTGTTAGAGTTTTATCCTGAGATGGCAGCAGTTCAGTCCCGTCTTCAGGCTCCA
CGTTAGGATGGCCAACTTTTTTTTTTTGTTGTTGTTGAGATAGGGACTTGCTCTCTTGCCCAGG
CTGGAGTGCAGTTGTGTGATCTTGGCTCACTGCAGCCTCGACCTGCCAGGTTCAAGCGATCCT
CCCACCTTAGCCTCCTGAGTAACTGGGACTACAGGTGTGCACCACCATATCTGGCTAATTTTT
TGTTTTAAGTAAAGGCAGGGTTTTGCCATGTTTCCCAGGCTGGGCTTGAACTCCTGGGCTCAG
GCGATCCACCTGCTTCAGCCTCCAAGGTGTTGTGAGCCTCTGCGCCAGGCCAGGGCGGCTGA
CATGTTAGCAAATGGCAAATCAGCGTAGTGAGGATGTGTGGACACCGCCCCCCCTGCTGGTA
GGAATGTAGCGATGCAGTCACCCTGGAAAGCAGTTCGGTTGTTTCTCAGAAAGTTCGTAAAC
GTGGAGTTGTCACGTGACCCGGCCATTCTACCTCTAGGTATAGACAGACACAAAAGAATTGA
AAACAGACCTTCAACCAAAAACGTGCACACAAATTTCACAGCAGCACCACTCATAGGAACC
AAAAAGTGGAAACAGCTGAAATGTCTATTAATGGATGAACGGAATGTGATTTATCCATACGA
TCCTCCTATCAGGAGGAGCGGGCGCTGCCCCTTGCTACGACGCGGATGGGCCGCGGGGACGT
CACGCTCAGTGGAGGGAGCCAGACGGGGAAGGCCGTGCAGCTTGCGATGCCCTTGGCAGGA
AATGCCCGGGGGGGGGAAGTCCGTGGAGCCCCGTACACCCTGGCGGTGGCCAGGGCCTGGG
GGACCAGGAACCAGGAGCGCCTGCTGAACGTCGGGGGTAGGAGGGGGTTTATTTGGGGGCG
ACGAAGAGGTGTGGAGTTAGATAGGGGCAGTGGTCGCACAGCACTGTGTGTGCAGGCGCTA
CTGAAGTGCACGCTTTAACATGGCTAACATGGTGAACTTCAGGATATGTCTATTTCAGCACC
GCAAAACAAAACAAAGGCATAGAGAACGGAAATCCAGCCAGGCGAGGTTCAGTCTGACGTA
CAGGCTCATGCCCCGAGCCGGGCTGAGTCCCCGCCCTGGGAGGTTGGAGGCTGAGGTCCCTG
GCCCTACCCATACTCCCAGCGCCTCAGGTTGCCCAGGGCCCAACCAGGAGGTCAGGAAGGTA
ACCCCCAGGACAGGTGATACCCGCCAGGGGCCAGGTAAGAGTCATGAAAAGGAAAGAACAC
AGCCTCCCTGCGAGCTCGCTCAGGGCCCCGAGTCAAGCCCCAACCGCTCCCCTCCTTCAGGC
CCCACCGTCTGACAAGCGGGCTTGCCTGTCTGTCCTACAGAGGGTAAACTGAGGCTGTGGGG
CTGATGCAGCACTGGGGCCTGGCCTGCTGGCCCAGAGTCCTGAGTGCCTGGCCTACAGCATG
GGGGTCCCCTCTGCCCCGTTCCCTCCACGCCCGGCCCTCCTGGGCGCGCCCCTCGCCGCCTGC
CCCTCGCCAACTGCCTTGTCTTTCCCTCCGCAGTCGGCCTGGAGATTCTACGCCACCAACCTC
TCGCGCACAGACCTGCACTCCACGTGGCAGTACTACGAGCGAACGGTCACCGTGCCCATGTA
CAGGTACCGCCGCCGGGCACCTGCCACCAAGCAACTGTTTCATTTTTTATTTTCCATTTGTTCT
TAAACCCCACTTTTTGTTGTTCATTATTTTGATTGATTTTTTTTCTTTAAAATGTATTTTTCACA
AAGGAGTTCTGTGTGTGGTTTATTTCGAGGCGTTGCTTCTGCCCTTTGTGTTAGAGCTGCGGC
TTTCTCTTCACTTTCATTTCCTCACCCCTTTGCTTTATCGTCCTGGCCGTGGGTCGTGCTGTGTT
TTGTGCGTTGCAAGGACGCTCCCCAGGGAGCTCAAGGTAAATGCCCCCCGCCCCGGGCTGCT
CTGGACCGACAGCTGCTCCCCGGGCTGCTCTGGACCGGCAGCTCGAGGTGCCCAAGCTTCAA
GAGTGGAACCAGTACCATGACCCCGTCCCCCATCAAGGACCTAGGGCTCCGGCGTGGGGGG
CAGGAAAGTAACCCCGGATCACAGTGAGACTTTCAGACCAGGAAACTCAAATTATCCTGGG
GGTCCCAGGTGTTCCCCAACAGGACAGAGCACACAGACAACCTTGGAGCCCCTCGGGCAGC
AGAGAAGGGGGCCTAGAAAGTGCCTGTCGAGGCAGTGCTAGCAGGTCCTGTGAGCCTGTGT
GGTCGGAGGGCGGCTTTCCCGTGTTTTTTTGTTTTACAAAAGGGCAGGTGGCAGCCAGCTGG
GATTTTCGTTGCCTGGCTCTTGCTTAAATTAAGGTCTGAGATGAGGGGTTCTGAGGGCCTTTC
TCCTGGTCCACGCGGGCCTGACCCCTCGAGTCCGTCTGTGTGGGCACCACCTTGCCCGCCCGC
CCTCTGCTCTGAGGTGGGGAACTGAGCCCCGGGACTTCCCAGCTCCCGCGCCCCCCGTCACC
TGCTCCTAGGGGGTTTCTGTCCCCTGGAGCCTTCCCTAAGGGTGGGGCCTTCCCTGTGGGTGG
AGCCTTCCCTGAGGGTGGATCCTTCCCTGGGGGTGGGGCCTTCCCAGAGGGTGGAGCCTTCC
CTGAGGGTGGGGCCTTCCCTGAGGGTGGGGCCTTCCCTGAGGGTGGAGCCTTCCCTGTGGGT
GGAGCCTTCCCTGAGGGTGGAGCCTTCCCTGAGGGTGGGGCCTTCCCAGAGGGTGGGGCCTT
CCCTGAGGGTGGGGCCTTCTCTGTGGGTGGATCCTTCCCTGAGGGTGGGGCCTTCCCAGAGG
GTGGAGCCTTCCCTGTGGGTGGAGCCTTCCCTGTGGGTGGATCCTTCCCTGAGGGTGGAGCCT
TCCCTGAGGGTGGGGCCTTCCCAGAGGGTGGAGCCTTCCCTGTGGGTGGATCCTTCCCTGAG
GGTGGAGCCTTCCCAGAGGGTGGAGCCTTCCCTGTGGGTGGAGCCTTCCCTGTGGGTGGATC
CTTCCCTGAGGGTGGGGCCTTCCCTGAGGGTGGGGCCTTCCCTGTGGGTGGGGCCTTCCCAG
AGGGTGGAGCCTTCCCTGTGGGTGGATCCTTCCCTGAGGGTGGAGCCTTCCCAGAGGGTGGA
GCCTTCTCTGTGGGTGGAGCCTTCCCTGAGGGTGGGGACTTCCCAGAGGCTGGGGCCTTCCCT
GTGGGTGGATCCTTCCCTGTGGGTGGAGCCTTCCCTGTGGGTGGATCCTTCCCAGAGGGTGG
AGCCTTCCCTGTGGGTGTGGCCTTCCCTGAGGGTGGGGCCTTCCCTGTGGGTGGATCCTTCCC
TGAGGGTGGGGCCTTCCCTGAGGGTGGGGCCTTCCCTGTGGGTGGGGCCTTCCCTGTGGGTG
GAGTCTTCCCTGTGGGTGGATCCTTCCCTGAGGGTGGAGCCTTCCCTGTGGATGGGGCCTTCC
CTGAGGGTGGGGCCTTCCCTGCGGGTGGGGCCTTCCCTGTGGGTGGATCCTACCCTGAGGGT
GGAGCCTTCCCTGAGGGTGGGGCCTTCCCTGAGGGTGGGGCCTTCCCTGTGGGCGGGGCTTT
CCCTGAGGGTGGGGCCTTCCCTGAGGGTGGGGCAGGTGCACAGTGTGGCTGGGAATTCTAAG
CTGGGGTGGGGCCCAGGATTCTTCTTGAGGATCCTGAGTTGGCCAGGGTGGACAGACCCGCG
GCAGTCCTCAGTTGCTACCTGCCTTTTCAGGGTGTCACTTGGGAGGAGGATGCCAGAGCCCTT
GGCGGTCCTCGATGTTTGCAGTCGCCCTCTGGGGTCTGCCCCAACCCCTCCCTCAGCACCATC
CCCCCTGCACCTCCTGGAAGCCCGACCTTAGGAGCCTGGCCCTGCTTGTCTTTCCTGGGAATG
TCGGCCATGCCCTTGTTTCTGCAAAGTGACCTCTCTCAGCCCAGGGCAGGGACAGAACCAGC
CCTTGTGCTAATTTTCTTTGTTCTGTTGTTTCCCCTACTTTCCTCAACTATTTGTTTCATTTTCTT
TTTCTTATCCCGTTCTTTTTGAAATTTTCCGTTCTTTTCCCTTTGTGTGTGTGGAAACACTTGAA
AGTTCGCAAACTCAAACCTACGGGGCCTCCAGGTAGGAAATGCATGGACAGAAGCCCTGTGT
GTGTGTGTGGTTCTAGTTCTGTGTCTGGAACTGTGACCGGGCAAGCCCCTGGAGCCCCGTGG
CCCACCCGGTCTGCAGAGTGACCACTGTCCCTGCCCACCGAGCATCCCTATGCCCCCTGCCCT
GCGTGGCCCCATGCTGGGCGGCCAGATCACCCTGGGCCCTCCCCTGCCTGGAGCCCACGTGC
CAACCCTGCCCTCCCAGGGCCCATCCACCCTTCTCGTCCTGCCTCCCCCCAGCCGCTGCTTCC
TGTTGCCAGCCCGCCTGTGTCCCCCGTGGTCTCGTTCTGTCCTGCTTCTGGCCTGGCTCCCCTC
GTGCCTCCATCCCCACCCCACAGGCCCCATCTGTGAGCAGAGCAGGAATGATGGATGGGGG
GCTTCCCCTGCCCTGGCGCTGGGCACCCATCTGGGGCCAAGGGTGCTCCCAGGCCAGGACCC
CCAGACCAGCCACAGTCCTTGTCCCACCGTGGCTCACAGGGCAGCCTCTGCGGGCCAGCTAG
CCTCAGGGGTCTGCCTCAGGGTCTCCTGCCCTGGACTCAAGTTTCTCCTGAGTCTTGCCCTGG
CATCACAGGGGAACCCCAGGGCTCCAGGCCGTCCCACCCCTGTCCCCACTGCTGAGAGCAGG
AAACAAGGCTGTTTGTTCAGACTGGAGAGCGCTGAGCTGCTATGGGAAGCCTCACCCCCATA
CAGCACAAAGGGCCTTTACCTGTCGGGTGGGCAGGCTTGTGCTCTGTGATGACACTGCCCTG
CAGAGCCTGGGGCCAGCCACTCCCCACATAGACCAGAAGGGCTTCCTGCACAGAAGCCCTTG
ATGACTCCTCCAGGGGCCAGTCACCCGTCCTGGAGGAGAGACCTGGCTGCCCTTCCTGGGAA
CTGTTAGAATGTTGACTCTCCATTCCCAGAAAGGAAGTGGGTGCAGTGCTGGGCAGTGGTGG
GTCCAGGGCTCCTCTGCCCCCGACATGCTCAGCCATAAGACATCTCCAATGTCTGCAGAGGC
CCTGCTGCCCCTGGGGCCAGGAGGCTGGCGCTGAAGCCGGCTGCCTCCTCCCCAGCCTCTGC
TCTCTCCCAGGGCCGCCCCCTCCCCTTGTTGCACCCTCGTCCCACAGCTCCGTCCTGCCCTGG
CCCTGCCGCCCGCCTGTCTGCCCTGGCGCTGCTGCTCTGTGTGTTCGAGGCATGGCCTCCTAT
GTGGCTCTGAGGGCTGACGTGTGTGTCTCTCACCTGGGCCTGCGTGCGTGTGGGTCCCATCAA
GGAGGGGTGGGGAGCCACCTGTGGCCCCCAGACAGACTCCGGCCAGGCTGGGCTCTGCTGC
GCCCACAACACCAGCTCACCAGCTGCCTGGTTCCTGCTCCATCCCGACTTGGACAGCGGACT
GTCCTCTGCAGGGAGGGGGCTCACAGGAGCCCCACTCCTGCCAGCATTTTGGGGGCACCTCT
GGGGCTGCAGAGGGAACAAAGAGGGGCCAGGCACGGCTTTACCAGCTCACACCTGCCTAGC
CTGTGGGGGGGCACCAGAGGCTGGTGGGGTGCAGGGAGGGGCTTCTGGACAGCTCTGGAGG
GAGGAGTGTGGCTGGACGAAGTGGGAGTCGGCAGTAGGGTGATCACCAAGCCCTGAGGGAC
AATGGAGTCAGCCATCCTTTTGCTTTGGAGGCAGTGGGGAGCCATTGAGTGTTTGGTCAGGA
GAGTGGCAAGGATGCCCATCTGGAGGACCAAGTAAGCCTGGGTGGGCAGAGGCAGGGCAGG
TGAAGAGTCTGGAGGATGTGGGCCACGGCCCAGCCTGGGGCAGAGGGAGGACACGGGCAGG
GGCATCTCCAGGGGGTGGCCGTGGCCATAGCCGCGTAGGGAGTCAGCTGGGTGAGGGCTGG
GCTGGGAGGTGGCCGTCCTGCCTCCAACGCAGAGGAGAGACCAGGACGTGGCCCCAGTGGG
GAGTGCTCCACAGGTGGGTGTGGGGAGGGGTGGAGGCCGTGGATGGGATGGCAGGGCCTGG
GAGCAGAGGCTGAGGAGGCGGAGTGGAGGGGGTTCAGGTGGGTCAGGGAGCCGGGTGGGG
GAGGCCCCAGAGGAGGGTGTCTCTTCCTGAAGCGAGCTGGAATGCTGTGAGGGCATGGGTG
GTGAGGAGGAGTGGGCAGAGTGAGGTGGGTGGGAGTGGGGAGGGGTGAAGAGGGGGACGT
CCCACCCAGACGTGAGGAGGTGCCTGCATACCTGCCTGGGGGGCTTCGGCGGGCCCGGACTT
GGTCCTGCTGGTGGCTGTGGGCGGCAGGGCAGGAAGACAGGGGTGGGTATGACCAGGACTG
GGAAGGGGGCTCGGAGCAGAGCTGGGCCACCTGCGGAGGATTCCCAAGGCATCTGGGCCTG
ATGAGGCTGGGCTCATCCTATAGACTCCAGAATGAGCTGCTCTTTGCCAAGCAGAATCCTTTT
CTGTATCCCCACCTCCCAACTCCTCCAGCTTTGGCAGAGAAAGTGGCTGTGTAGAGAGGGGC
CGGGGCCCTAAATATATTCGCGTGCTGTCCCTCTAGGATGTTCAGGGATTAGAAGGGGGAGT
AGCGCCTCCCCCTACCTGCGCGCCCTCCTCCACCTGCTCCAGGCTGCAGGGTCGGGGCTGCA
TGGGTGGCCAGGCGCCCTCTTCCCTCCTCCCTGCTGTCCCTGCTGGTTGACAAGGCCCCAGGC
ACAGCCAGAGAATGAAGATGGCTCCCAGGCCTGCCCAAGAGAGGTGGGCCTGAGCCCAGGG
CCCAGTGGGCAGCTGCCTGAAAGCCTGGCCACGTCACATGCCGCCGGGTGTCTGACACTGCA
GGCGCCTGCCCATGGAGCTGTGCAAGCAGAGGGAGGTGTCCCAGGACTCGGGAGGGTGAGA
CGCTCACTCCCCTCTCCTTCTCTTGCCCCAGACTTATCCCCCCGCTGAACCAGCTGGAGCTGC
TGAGGAACCTCAAGAGTAAATCTGGACTCGCTTTCAGGTCAGCTGGGGAGCTCCAGGTGGGG
CGGGTGGGCGTCTCAGTCCTCCTGGGGGCCCCAGCTGCCCACAGAAGACACGCCAGGACAG
TGCCCCAGGGACTCCAGGGAGCTGGGACGCGCGGGGACGCTGGACTTGGTGATGGTGGTGC
TTTCTCTTTTCGCGTCTGCTGACCTGGTCTGGGCTCCCACCCCCAGAGGGGCAGCTGGTTCGG
GGGACGGGCAGAGAGGGAGGGGCGAAGGTTGATATGGGGCCGGGGGCTCTGCATGGCTCCT
AATGGGGCCCAGGCGCGTGGGTCGTACGACAAAGTGTCCCAGCCCGTCGTCCTCTCGAGTGC
CTGGTGGTAACTCAGGTGCTGATGGCGCCTGCTGCCTCTCCTTCAGGCCCAAAGCCCACGGC
TGTCAGAGTGCCGGCTGTCGAGGTAGGTTGCAGAAGGGCCTGCTGAGCAGGAGAGGTGGAA
GGCGAATGCACCTGCCAGGCCTGCCCAGGCCCAGAGCCGTCCCCAAGCAGTTCCATGTCTGA
AAGCCCTTGGTGGGGCAGGGGCTTCCAGTGGGTTGGATGCTGGTTTCTGCCTCCTAGAGACA
CTCTTCTGCTCTGGAACCTATCAGGACGATCTCAGATCATCCTGGATTTTCCGGGGGGCCCT
GAGTCCAACAACGAGTGTCCTTACAAGGAGAGGGAGATTTGGAAGCACACAGAGGTGATAG
CCAGCTAGGATGGAGAGAGAGACTGAAGTGATGCGGCCACAAGCCAAGGGCTGCCTGGAGC
CACAGAAGCTGGAAGAGGCAGAAAGGACCCTTCCCTGGAGCTTTCGGAGGGAGCAGGCCCC
GCCCGCACCTTGATTTTGGACTCTGGCGTCTAGGACTGTGAAAGAACAAACATCTGTTGTTAC
ACACTCCAGCGGCACTTAGTTACAGGAGCCGTTGCACATGAATCCAGACTTTAATACCGGGA
GCCGCGTGCGGCTGTGGCAAACGCCTACAAGCGCGGGTGTGACATCAGAACCGGGTGAGCC
AAGGGCTCGTTTCCTAGAATCACAGGTTCATGCGTGGAGGACTCCAGCCCCAGGCTGATGCA
CACCTGCAATCACACACACCTGATTTAAATGATTCTGATGACTTTATTATTTTATTTATTATTA
TTATTTTGAGATGGAGTCTTGCTCTTTCGCCCAGGCTGGAATGCAGTGGTGCGATCTTGGCTC
ACTGCAACCTCCACTTCCCGGGTACAAGTGATTCTCCTGCCTCAGCCTCCTGAGTAGCTGGGA
TTACAGACACCTGCTACTACGCCCGGCTAAGTTTTGTTATTTTTGGTAGAGATGGGGTTTCAC
CATGTTGGCCAGGCTGGTCTGAAGCTCCTGACCTCAAGGGCTCTGCCCGTCTCGGCCTCCCA
AAGTGCTGGGATTACAGGCGTGAGCCACTGTGCCCGGCCTCTGATGACTTCAGATGATGAAA
TTTGGGACTTTTTGAACTGATGGGATTTGGATGAGATTTTGGACGTGAGTATTTTGAGGTGCA
GGCTGGAAGAAACTGAGGTGTCCTGAAGAGATGGTGGGTGGGGACACGGATGTTGAAGGTG
CTTCTGGTGCGGGCTCAGGAGGAAGTGAGGATGCAGGTGTTGGAAGCTGGAGGGAAGGCTG
TCCTTGTGTTGTTGGTGGCAGAGACTCCACTGGGCTGTGCGCTACTGTTGGGCAGAGGCAGA
ATTTTTAAGTGATGAACTGGGATATTTAGCCAAGAAGATTTCCAAGCAAAGTGTGGAAGGTG
CAGCCTAATTTATTTCTGCTGCTCATAGTAAAGTATGAGAGGAAACAGGGAGATTCAGAGCA
GCACCTGGCCGATCCGCCAAAAGACTATTAAAAAAAAAAAAAAAGGCATTCAGGGCAGAAC
TGCTGTAAGAAACCAGAACTTGAAGGTTTGGGAGGTGCTCAGCCTGTCCAGCTTGCAAAGGG
TCTTAAATTAGGAGATTCACTGTTGGCAAAGCCTGCTTTGGAGAGAAGCCCACAGGTGTGGC
CAGACAGCCTCATGCTGAAGGCACCAGGTGTGCGGCTGCAGCCTCCACCGTCTCCACAGAGG
CCTGAAGATGGAGCTATCGGGAAGGATGTGCAGAGGGGCCTCTTGTCTAACGGCGTTGCGGC
CTGTGGCATATTCAGGAGGCCCTAGAGGTTTTGGGGAGTTTCATACCAGCAGAAACTAGATT
GGCAGAGACAGCGTTGGACGAAGGTAAGAGGGCATCGAACTTCGGGAGTCTGCAGGCAGGA
AACAGGCCGGTGGCGCTCCTCAGCTGTGAACACGAGCTGCCCTTTAGGAAACGGGGAACGA
TCCCGAGGCTCCCGCAGAGGCCGGCGGGGCTGAGAGCCCCCGAGGAGCGTCCCCAGAGCTG
TGCCACGTGGAGTTTGCCCCGCCGGATTGGAAACGTGGTGGCCGGCGGCCCCTCTGTCATTC
TCCCTTTTTGAGTGGGAAGAGCTGTCCTGCTGTTGATACTTTAAGCAGATGGCTTGTTTTCTA
GTTCACAAGCCCATGCATGGAGGACTTTAGCCCCAGGTTGGCGCACACCCACGGTCTCACAC
ATACCTGATTTAAGTGATTGTCATGACTTCAGAGGATGGGATTTGGGACGTTTTGAATGGGT
GGGATTTCTTTTTTTTTTCTCTGAGACGGAGTCTCCCTGCGTCACTCAGGCTGGAGTGCAGTG
GCGCAATCACAGCTTACTGCAACCTCTGCCTCCTGGGTTCAAGTGATTCTCCTCCCTCAACCT
CCCGAGTACCTGGGATTACAGGCACCTGCCACCACACCCGGCTAATTTTTGTATTTTTAGTAG
AGACGGGGTTTCACCATGTTGGTCAGGCTGGTCTCGAACTCCTGACCTCAGGTGATCCACCC
GCCTCGGCCTCCCAAAGTGCTGGGATTACAGGTGTGAGCCACTGTGCCTGGCCAACGGATGG
GATTTCGATGAGGTTTTGGACACACAGTCCATGCTGCAGAGGGTGGAGATTTTGGGGGATCT
TGGCAGGGGTGAGTGTATTCTCGATGTAAGATGGACATGAATTTTGGGGGGTCGGGGGAATG
CAGTGGTTGAATGCTGGTCTCCAAAATGTCCCAGAATTTGCAAATGTGACCTGATTTGGAGA
AAGGGTCCTTGCAGCTGTAACTGAGCTTGGGATCTTGAGATGAGGTCATCCTGGAACATCCT
GATGAGCCTGGTGACAGCTTGAGCCCTAGCCCGGTGACAGCTGTCCTTACAGAGGTAGAGGA
GGAGAAGACACAGAGACACAGAGAATGTCGTGTAGAGACGGAGGCAGAGACGGGGCGGTG
CAGCCACAAGCCAAGGACACCTGGGCCACCGGGAGCTGCAGGAGGTGATGAGGGACCCTCC
CCGAGAGCGTGGACCATGTTCCACGGCAACACGGTCCCTGTCACATGAAGTATTTGGAAGTG
CACCAAAGAGCCTGCTTGTAACACGGCGTACCAGCCGGGGCACCAGCCCAGGTGTTCGAAT
GAGATCGCTGGGGCCTCCCCATGGATGAGCCAAATCACGATCCGCCGATCTCTCACTCAATC
CATAACGCCTTTACCCGCGGAAACAGCCCCAGATGAGACGCCGGAAGGCCCAGACAGCTTT
GCCTGCAGGACGAGGCCGGGACAGTTGGTCAGCGGTGCAAACCCTGGGCTCACTGACTGTG
GCGCAGTGGAGCCACCCCTGGAAAGTCACGCGGAAGAGAATCATTCCATTAGGATGAGCGT
GGAAAAAAGAGTCGGAGCAGCTCACTCTGTCTCGAAAATTGATTGTAAATTAAACTCCCTTC
CAGAGCTTCCTTCAGCAAACACGTGCTTTTTGGAACTTTATTAATATTGGTGTGGTTCTGATT
AAAATGAGTGTCTGTAAATGTGGAGGGGTAGAGACCCCCAGGTCAGAAGAGCAGAGGCCCC
CGCCCCATGGAGTCCCTCTTCCCTCTCGGAGCTGCCTCCGTGGGCCCTGGGGTGCCCGTTCCT
GGGGCCGTGGGCTCTCTGGGGTGGGTGGGCCAGCTCCACCCCTGCCCAGCCTTTTCTGAGAC
CCCCCTCCCCTGCAGCCCCTGACCCATGGGACTGTGGGGGGCTGGACTGGTTGGGGGACCCT
CATGGGTGCTGAGTGGGCGGCCACTAACTCTCTGTTGCTTCTGTTTGAAGGAAGGACCCCCC
GCCGGAGCCGTCTCCAAGGTCAGTGCCCCCTGCTGCTACCCTGGTGTCTGCCGTGTGCACGG
CCGTCTGGTCTCTCTCCCACACGTGTGCGCAACCTGTCATGGAGATGTGAGGGCCTTGTGTGT
GCTTCCGTGTGTGACTGTGTGACTGCGGGTCCAGACCCCCGCCTGGCGGTGATGTGGGCCCTT
AAATCACTCTTCCTGCTCACCCCCTCCCCAGTGATTCGGTTTTACTTTGCAGCACTGTGGATC
CGGGCAGCTGGGCGGCTTCTCGGGGGTGGGGGATCCCCCACCCCGCCCACAAGTCTGGCCCC
AGGGTTCTCGGAGGCAGGGGGTCTCTGTTAGTGCGCTCCCTCCAGCTGCAGGCACATAGCCC
GAGCTCACAGCTGGCCTGAGTCGACGCCGGCTGGGGTGAAAGCTCCAAGTGGGCCTCTGGCC
TTCCCGCTGCTCTGGGTCCAGAGTGTCTGGAGCATGTGGCACAGACCAGGGCCCCTCGTCCT
CCGAGGAGGGTGGGACATCCTCTCTGTCTCACGCCCCTGGGTGGAGATTCTGGCTGGCCTCC
TCTCCCTGTTTGCCAAGGTCAAAGTGGGCCAAGGGTGCAGGTGCTTAGCCTGGTTCCCTCTCC
CGGGCCCCGAGGTTCTGTGGGTCGGGCAGATTGGAGACAGGACTCGTGTAAGGGCTCTGCTG
GGGTGAAGGATGGAGACAGAGAAAATCAAGATCCTTTCACAAGTTAATTCTACGTCTGCTGA
GCCCCAGCCCCCGACACATCACCCTGAGGAGGTGCTAGGCTTCTCTGGGCCCCCTGTGCCCC
ATCCACATGTTGCAGAGTAAATCTGGCCCCTTGGACCTGGGGTCCGAGATGGACGCCTGGCT
GCCCCTCCTGGACTGCGGGTGACAGCTGGCGAGACACTGCGGGGCTTGGGTGCGGGGAGAT
GGAGTGGGGCTGAGCTGCATTTTTCCAGCCACCCCACATCCCACAGAAGGGGAGTCATGGTC
AGTGCCTTGAGCTGGAAAGACGGGCAATGCTTCCGGCCCACACCAACCAAGAAAACCACCA
GGGGCTCATTCATCCTCTCAAAGAGGCTAAGAAAACGGACGAGGGCCCAGAAAGAGCTGGG
TCGGCAGCGTAGCCTCTGCTGGTGCCAGCCCCCAGTGCTGCCACTGCCTCCCCCGCCCCTGCT
GCTGGCCCTGAGGCTGGGAGTGGCTGTGGGGATGAGCACTGTGCCCCCCACAATGGGCACCC
TTGGCTGTGATTTGGGCGGAACCTCGGTGCTGTCCCAGGTGGACGCGAGGCTTCCCGACGGC
TTCCGCGTGGCAGGGCCCCACGCCCTTGCTGGGAGCGACGCCGGAACCCGCCCCGTGCCTCA
CGGCCCCTCGTGCTCGCTGCCCCAGCCCGGCCCGCTCCACGTTCTCGTCTCAGCCTCTCTCTT
TCTCTCTCTCCCTCCCACCCTCCACTCCACCCATCTGGACACCCGCTGCCCCCAAGTGCTTTA
ATCCCTGTTACTGCCCTTCTCGGGCCCCTCTCTCGTCTGCCCTCTCTTTGGGGATGAGGTGCTG
GCCCTGAAGGCGAACTCCAGGTGAGGACACGCGGGAGCCTCCGGGCTCTCCGGTAAGCTCT
GTGGCTCCCCACCCCTCCCCCGTCTCTTCCCCATGTGTCCGGGGGGTCCTTGCCTGTGCCTCC
CACGCCCTCCCCCACGGGCGAGCACCCACCCGCCCCGGCCCTCCTCCTGGGACGCCTGTGGT
GGGGGACGTGGGGCTGCTGGGTCTGGGGCTTCTGGAAGGTGCCTGGGGTGCGGTGGGAGTG
AGGAGCAGTCTCTGCCCTTGAAGACCCTCCCCGCCCATCCTGGGGTGCTCACCTGGCAGGTC
ACGGGGTGGCATCTCTGTTCCCTGGCCTGGGGCAGCCGCAGCCTGTCCAGTGTAGGGTCGTG
CTTAGGAAGTTGTTCCGCAATCATTGCCTGCCACGTCCCTGGGCTGCTGGGTGAGGGGCCTG
CTCTGCGAAGCTTGATGGGGAGCTCGGCTCCTGGCGCCCCTCTGCTGCCTGACCTGGGCTCTG
TGAGTCTGGTGGGACCTGTGCCTCCGCCTGCCCTTTGGAGGTGGAGCCCGCACCCCGCATGG
CAGGACCCCGCTGGTGTCAGCTGGTCTTGGCAGGTGGATGGTGTGGACACAGGGACCCTCAC
TCACTGCTGAGACCCCCACCCCCATCCTGGCTCAGAACACAGGCACGTGGTGGCAGGCGACC
CGGCTGGCGGGGGCTCGGTTCCCGTAGGGACCACTGGGGCTGTGCTGGAGTCAGAGGAAGC
GTCTACGTTGGGGTCGGGTGGGGAGCCAGGCAGGCTGCGGGGCTCGGGCCGTGCTGCCCCA
GGGGGGTCCCGGGGGGTCTCTTTGGCCACGGCGGTTCTGACTTGTGCTGTCTTGTCCGACACC
CCATCTCACTGCCTCCTGCCCCCGTGCTGGAGGAGGAGCCCCCAACACCGGCTGCCCCCTCA
TGCCTGTGCGGGCTCCCCCGTGCCTGTTCGGGCTCCCCCTCCTCTTCTCAAGCACACGTGTCC
GCGTGTTTTTGGTGCATGTCTGTCCTGTCGGGTCTCGGTCAGTGTCTGTGGGTCTCGAGCTCA
CTGTGGGCCTGTCCGCCTGTCCGACCTCGGGTCACAGGGCCTTCTCACGCAGTGTGGAGCTC
CCTGTGGCTGATGGGGCAGGAAGGAAGCCCAGTGCCAGCTGCGGACCCCAGGGGGAGGTGC
AGCTGCACGGGTCCCCCACAAAGCTTTGGGGGTCTTGGGGGGCTGTGCCTGCAGCGCCTCCC
TGCCAGCTCTCCCCGGCTCTCAGACCACGTTGCCCCATACAGGAGCTGCCAGCCACATGTGG
CTGTTAAAATAACATGGATTATAACGGGCACGGTTAGAGATTCACTCTCGAGTCTCAGGTGC
CTGGTGGCGACAGCCGGAGCTTCAGTGTGGCTACAGGACGATCCCATTGCAGAATCGGGCGG
CAGGGCCGGCCCGGGAGGGGCCTCTTGGTCAAGGCGATCATGCTGGGGTCAAAACAGGAGA
CCTTGGGAGGTTTGGGGAAGCGGGACCCTGCCTCCTTCATGCTCTTTCCCTGGGGTGGGGAA
GGTCTCCTGGGGCCCTCCAGGCAGGGGCTCTGTGCTTGGGCCTGGGTGGTGCTGGTGGGAGT
CTGAGGTCTTTGCCTGCCAGGAAGGCCTGGGTGTCATCGTAGCTGGGGTGGTCACAGCGGGA
GAGACTCTGAGCAGCCAGAACCTCAAGCCAGGCGCAGGGAGCCGGCTCCCTCCTGCCTCGTC
CCACCGTCGCGTTCGTGGTTAACCAGCAAACAGAGTCCAGCCTTTGCACTACATCCCTTCGTG
CCTGGGGACCCCAAAGTCGCTTGTGTACTAAAAAGCCGGACCGGCACGGGGGCTGGGGTGG
AAGCCGGCCACCCGCCCTTGGAGTCTTGCAGGCTGGTGGGGCTTGGGGAAGGGTCCAGAGCC
CTCAGCCTACCCGGGCGGCTGAGTGCTGCCGTGACTGGAGCTAGCCGTGTCTGTGACGCCGC
TCGGAGAGCTGCCTGCGGGTGCGTGCAGGGACATCGGTGTCTAAACACACGTGTGCGCAGG
AAGACGGGAGGCTGGGTGCTTTCTTCCTAGAGCCTGTGTGTTCTGAGTGGATGCCGTCGCCA
CGCTGGGGTTCCTGGGGCACCGAGTTTGAGAAGTGCTGTCTCAGGAAGCAGTGGCAAGGAG
GTTGGGGGTGGCTGGGGTGGGGAAAAGGAAGAGTTCTTGAGTTCTTGGTGATTGGTTTCATT
TTTTGCTTAATCATTTGCACATTGTGATGCAGAGCTAAATCGCCAGGGTTTTTGGGGAGATTC
TTGGGAAAACGTTCCGTGTTTTCTGAGCCTTTTCTAAGGAACGCCCCGGCGGGGAGCTGCGC
TCCTGAAGGGCTTCACAATGAGGGGATCCACTGAGCCCCAACGTGACTGTCCTCAGGGGAGC
CCTTGAACCCCCAGATAGCCATGCCTGCAGTGGCAGCCACGCCTCCTGGGTAAACCGAGACC
CACCTCCAACTGGGGGACTGGCTGGCATGGCCAAACGTCGCCGATTGGTTTGCAAAGCGGGA
CAACTGCACGTTTTCCTCTCTCAAATGTGTGTGCTTCACACCCACGCCCTTTCCCGGGGTCAC
GTCCTTCCCAAGCAGGGCTTCTGCGGCGTCTGAGGCCACATGTATTATTGTTTTGTCAGCATG
AATACTGCAAAAAATAATACATCTTTTAAGACATGAACCCAAACTGTGGCTTTGGAAGGAAA
AGGTATTTTTGGAGGAAAAAAAAAACAGGGATTTTTTTCCTTCAGAAATAGGGAAGTGTTTT
CTTCTCCGGACGGTGGAGACGATGGTGCACCTTCCTCGCTGGCCTTGCTTGGGCGCAAGCTCT
GCCGCTGGGGACTGCACACAACACCCTGGGTGGTTGCAGTGCCTCAGTTTCCCTGCTCCCGG
CCGTGTGCTGTTGCCGGCGCCCAGGACTAACTGTGCTCTCCTCATTTCCAGTAAAGGCAGCCC
GTGCAGAGGGCCCCTGTGTGGATGCTGCCCCGGACGCTCTAGGTACCGCGGAACACGCCGCA
CGGACTGACGGCTGCTGCACGGCTCCTCTGCCCCTCCCTGCTTACTAGAGGTTCTGGAGGAG
CCAGGGGGAGGCTGGCGCCCCAACCCCAATTCTGACCCACCGCCCTGGCGGTGCGGGCTCAG
CTCCCTCTATTCTAGCCGACAGCTGCGTGCAGGGCGGAGCTGGGGGAAGGTGCTTCTTCTTG
CTCCCCACGCCAGGCGATGTCCTGCGCACAGCCAGAGGGGCCAGCCAGGGGCTCTGAACCC
AGCAAGGCGTCCGTGCCTCCTGGGGTCCCCGACTGCTCTTCAGGGAGCGTTTCCCCTGGGGC
TGTCACTCTAGCACCTCTGCTGCGTACCTGAGGATGACTGAGCCTTCCTGGCTGCCTCGACCG
CGGGAGGCAGCTCCGTCTGTGTGTGCCGGGCTCCTCAGGGTCCTGATGCATCAGAAGCCCCA
GAAAGCCAGAGGCCCGCGGCTCCCTCTCCCAACAACAGGCCTGGTTTAGGGCAGGTGGAAA
TAGGACGGGGGAGGAGCCGCCCCCTTTGCACCGTGGGCTCCTGGCCTGCACCGTGACCCGCA
CCTCCCCTGTCCCCGCGTCCCCTGTCTGCTGTTGGGTGTGTGCAGGCCTGCGCTGGGGGCCCC
TCCCTCCTGGCCAGCACAGCCCAGCAAGGTGGAGGTGGCTGTGTCGTGCTCCTGTGGGGCTC
TCCTTGGCATGTGCATGCCGCCGGTGGTGCTGGCCTTGGAGGGCTGGTGGGGCTGCATGCTG
CCTATCTGCATCCGTGCATGCTTCGGACCCTGCTCCATGGGGGGCCCGCGACTCCATGCCACC
CTCTCCCTGTGACTTTGCTGCTTGTGTGCTTCTGGGGGCGTTGGTGCTTGCGGGGAGACTGTT
GGCAGCTCCAGAGAGGGAGACGGGCGTGTGGGCTGCCTGGGGCATGGCCTGCTGGGGCGCT
AGCAGCTGTCCTGCGTGTCCGGAGCATCTATTCAGGGTGCTTCTGGCCGCAGGCTGGTCCCTC
AGGGCAGGGGCTGAGGTCAGGCTGCTTGGGGACCAGAGGGGTTTGAAAGTGGGGGTGGGAG
CCCAGGCCAGAGAACAACCCAGGGCAGGCCCTGTCCTGCTGCGAGCAGCCCCCACGGGAGG
CCCCTTCATCCTCACGCTTCCTGGGCGTCGTTGGTCCGGACACCAGCTGGGCAGGGGTCTGG
ATGCTCCTCAGCCTCCTGTTTCCTGCGGGCACCGCACATCTGAATTTCACCATTGGGAGGGAG
GGTGGCAGGCCGGGTGGGGATGGCGCGCTCTCAGGTGCTTCTTGAGCCTGCCCCGGCCCCGT
AAGCCCCAAGCGTAGGTGCCACGGACGCCCCTCGCACACAGAGGGCAGCTCTGGCCGAGGG
CTCCTCCCAGGTGGGTGGCGGCAGCTTTTCGCGTGCGCTTGTTCTTGGCTTGTGGCCGAGGGA
GGGCCTGCGAGACCACGCGTGCTCCTGGCAGCTGAGGCAGGCTGGCGGGGTCGGGTGTCTGC
AGCATGGAGCCTCACCAGCTGTGCTTGGAGGGTGCAGAGTTTGGGTCATGGTGGAGGTCCAG
GGGAGGGAAGGACATGTCCAGGTCGCGCCACCCTCCTCTTCCTGGCAGAGCCTCCCTCGCAG
ATCTGAGGTGGGAGCGGAGCCCGAGACCCGGGACAGCAGGGCCGCGCCGAGGCCAGGGCA
GGCTCCTTCCCGCCGGCTCTGTCTGGGGCAGTACACGTGGGCTGCGTCCCGGCGGCCCCTGG
GGCTGTGCTGGGCATCGGGTCCAGCAGGGTGTGTCTGCCGCTGGCGGGGCTGTGGCTCCTTG
TCGTGGTCAGCGGGGGGTCCCCCCTGAAAAATGGGAAGCAGACGTCGGGGCTGGGTCTTCTC
TCCTGGGGCCTGTGGAATCCACGCCACTCCCTTCCTGCAACTCCCTGCGGACTGTGGGGCCTG
ACCCTTCCTGGCCCGCACGTCTGTGTGGCCGCCTGGGTATTTGACCCGTTCCCCTCCGGGGCC
GCAGGTGGGAATCTGATGCCCATTCCTCAAGCTGTGCCTTTCCCACTGCAAGCCCTCAGCAC
CGGGCTCTGCCCAGAGACCAGCCAAGGGCAGCCCCGAAGCTACAGTTGGGCCGACCGGCTC
TGTCCTCCACCCGGTGCTTTGCCAGCTGCCGTGAGAAAGGAGGTGGCTGCTTCTCTCTCTGGT
GACTGGACCTGCCTCGGGTGGAAACGCAGAGAGAGGAATTGAGTCTTCCAGTGACTCCCACT
TGCCCTGAGGCCTGCCTGAGCGGGGTCAGGAGGGGCCTATTCTTGCCTTTGAAATGGCTCGT
GCGGTGGAGAAAGAGAGAACATTCCAGGAGTCCCGCTGAGCCTGTGCCCATGTGCTCCCCTG
GCCCCCGTCCGGTTCTCCCCGACCACCTGTGCTTTTGTTGTTGGGATGGGGACCAGAGGCCGT
TGGGACAGAGGCACCCACAGCACCAGGACTCTGAGCACCCCTGGCCCCCATGGGCACGGTG
TGTTGGCCTCAGGCAGTGCAGGGGTGTGGACTCAGGACTGGGGTCCTGAGCGAGCCTGTACC
ATCGGCTCCTGACGTCCCCAAGCACGCGACGCCTCGGGACTCGGGGGATCTCGGCCACTCCG
TCTCTGCTTCCCCGAGCGGGCACAACACCCAGGGGCTTCTTTCTTTCTCTCAGAGACGCCTGA
GTGGGGACAGATGGTCTGCTGAGTGGGCACAGATGGTCTGGGCGGTCACCTGCTGCCTGGGG
GATGGGGTGGGGAGGGGCAGCCAAGCGATCCGCGTGTCCAGGCCAGGCAGACGGGCGCGGT
GGGCTCTCATGTCTGTGGCTGGTTATCTCTGTGCCGTCTGGTGGGCGTGTCTCTCTGGCTGTG
GGTCTGGAGTCGGGGCTGATGGTGGCTGCTGACCGGGGCCTCAGTCCCTCGCTGTTGGCTGG
GGAGCCGCCGTGGGATCAGGGAGCCCGTGAGGCACTGGGCTTCCTCTCTGAAGTGTGTCAGG
GATCTGCGGGGCCTCCCCACCCGAAGGCTTGTTTCAGGCTCCAGCAACAGTGCTCTGGGCCC
TGCCCAGAACCACCCTGAGAAGTTCCGTGGCTGCTAAGCATGGCTGGGCATGAGCGGGTCCC
CAGTTCGTGAGAGCCCCACTGTCCTCAAGGGAGCCAGGGGGTTCTGATGCTTTCCAGACCTC
AGGAGGCCACATCTGCCTGGCGTCTCCCAACCACCAACCACAGTGAACCCTCCCCTCCCCTC
ACTGTCGCCCGGCGGACGCTGGAGTTGAAGAACTCGGGTGCAAACCCATCTCCACGGTCACC
AAAAAACTCGGGGGCAAGGCAGGCCCAAGGACCCCCCGAGAGTATGGAGCTGGAGACCCA
GCCAGACACAGCTGCAAATCCTGCTTCACGCCTCGCAGGCCGAGGGGGCCGGGCCACAGCG
CTGTGGCTTTGGAGGCTGCCTCCCCCTGCCCTGGGGCAGGACTTGGATAGTGAGGCCCAGCT
GGCGGAGGAGCCCCGTTATCCATTCCTTCCTTCCAGAAGCCGGAAGGAGCAGTTCTCTGACA
CGGACCTGGTGGGAGGGGAGCCTGACAGGGGAGGCCTGAGCCCTCCTGAAGCCCATCCCAC
CAGGCAGGCCCCCGGGGTGGGTGGAAAGGCCGGAGTGTGGCTGAACAAGGGTCCACCGCAG
ACTTGGCCCCCAATGTCCTTGTTGTCCCCTGGCCCCTCGACTCTGGGGCAGAAGGAGAGGGA
CAGGCCGTGGGAGGGGAGGAGGGAGGCTGCTTCCTGGGAGCAGTTACGGGTGTGAAAAGGC
CACAGGGGAAACCACGGGCGGGGCCGGGGCAGACAGAGGTGCCCGAGGAAGAGCCACAGC
CCCGGTGTTTGGGATGCCCGTTGCCCTGAGTGTCACTGGGTGAGGGGCTGGCAGGACAGTGT
GAGGAAGGCTCCGGCACCTCGTGGTGGTAGCGGAAAGTGATTTAAGAGTCCACAGACATCA
ACTCTCCCCACATGGCCAACCTCTGGCCACGCCTGAGCCAGCCGGGGGAGCTTGCTGCCCAC
CCAGCCTCCCCTCCCTCCCTCCCGCCGGGTGCTCAGTGTCTGTGGCCTCCCCTCCCTCCCTCCC
GCCGGGTGCTCGGTCCCCGTGGCCTCCCCTCCCTCCCTCCCGCCGGGTGCTCGGTCCCCGTGG
CCTCCCCTCCCTCCCTCCCGCCGGGTGCTCGGCCCCGGTAGCCTCCCCCCCTCCCTCCCGCCG
GGTGCTCGGTCCCCGTGGCCTCCCCTCCCTCCCTCCCACCGGGTGCTCGGTCCCCGTGGCCTC
CCCTCCCTCCCTCCCGCCGGGTGCTCGGTCCCCGTGGCACCTGCTCTGAGCCACCCGTGTGGC
TGGCTGTTCTTCAGACCTGTGTCTCCCTCCGGCGCATGGGCGGCGTCGGCCATGAGCAGACA
CGGCCTGTGTTCTTTGCAGAGTGACTTCTCTCCCTGTTTTTCTGTCTGTCTGTCGGTTCCCGTG
GGAGCAGCCAGAAGGTCAGTTTGAAAGATCGTGTCTTCTCCAGCCCCCGAGGCGTGGCTGCC
AAGGGGAAGGGGTCCCCGCAGGCCCAGACTGTGAGGCGGTCACCCAGCGCCGACCAGAGCC
TCGAGGACAGCCCCAGCAAGGTGCCCAAGAGCTGGAGCTTCGGGGACCGCAGCCGGGCACG
CCAGGCTTTCCGCATCAAGGGTGCCGCGTCACGGCAGAACTCAGAAGGTGGGTGTGGCCGCA
TCCTCTCCTGGTCCATCCTCTCCGGGGATGGATGTGGTGGCCACGCTACTGTGGCCACCCTGT
GGCGTCGCAGCTGCTTTTGCTTGCTCTTGGAGTGGGAACCCCCTATCCTTTCTAAAATTGAGG
TGAGACTTGTGCAACCTAAAATTAACTATTTAAAAGTGTAGGAATTCAGTGGCATGTAGCAC
GCTCACGGTGGAGTTGTCACCACCACCTCAGCCTAGTTCCAGAACATTCTCATCACCACAGA
AGGAAACCATGACCCCATCAGTAGTCACCCCATTCCCCGCCTCAGCCCCAGCACCCGCTGCT
CTTGGGCCGTGCGTGTCTGCGGCTCCCACGTGGGGCCGTTGGGTCTGGCTTCCTTCACTGAGC
CTCAGATGTCAGGCCTGCACCTTCCGCCGTTAACATCTGTGCGTGGATGCGCCATGTTCTGTT
CATCCATTGGCCAGAATGTCCTTTGACGCCCCATATCGGCCACTCCCTGTGGCCACAGCCTGC
CGGGGAAGGTCCTGACTCGAGAGCAGCTGGGTGCAGATGGGTGGGTTTCCAGAAAAGCAGG
TGCACCCGAGGGGCACCACAGGGAGCCTCGAGGGGCTCCCTGGCGCTCTGTCTAGCCGGTGT
GCCCTGCAGGACCCCGGGAATAGGTCTCCCGGCCCCCTCGGCCCCTCACGGCCTGTCTCCTTC
CCCCCAGAAGCAAGCCTCCCCGGAGAGGACATTGTGGATGACAAGAGCTGCCCCTGCGAGT
TTGTGACCGAGGACCTGACCCCGGGCCTCAAAGTCAGCATCAGAGCCGTGTGGTGAGGCCCC
TGCCCAGCCGGGAGCCTGGGGGAGTGAGGAGGGGCCTCCCGCTCGGTGGTCCTGCCTGCTGC
CGGTGCTCATGGTGCTGGACAGAGCCGCCCAGAGACAGAGTCTACTGGGAAAAGAAAGGGC
TAAGACACGGGTGGACCTGGCTGAATGTCCTGGAGTGCACTCCTCTGGGACTCAGGCGGGTT
TCGGGTGGGATGCCGGTTCAGCAAGAGGCCTGAGGCCGGGCTCCACCTGGCTTAGGAAGCA
ACTCAGCCACCTTCTGCCTGCTGTGGCATGGAGTCCAAGGGGGCATGGCCAGGCCTGCAAGC
CCCACCTGTCCCGGGCAGAGCAGGGCCACATAAAGGCCCTTCTGTGTGTGGAGCTGGTGGGC
GGCAGACAAGAGGGGCAAGTCCAGCCATCTCCAGGCGCCGAGGTCTCAGAGGTGCTAGGAA
GGTCCTGGTGCCTGTGGCCGGGGGTCTCTGGCCCAGGGCTCACAGCCCCACCCACCCCCCTG
CAGTGTCATGCGGTTCCTGGTGTCCAAGCGGAAGTTCAAGGAGAGCCTGCGGCCCTACGACG
TGATGGACGTCATCGAGCAGTACTCAGCCGGCCACCTGGACATGCTGTCCCGAATTAAGAGC
CTGCAGTCCAGGCAAGAGCCCCGCCTGCCTGTCCAGCAGGGGACAAGAACGGGGTGGGCTT
CTGGGACAAAGCCCACTGTGGCCCATGGTGGGAGTGCAGGGGGTGTGTGGGCGGGGCCTCC
TCCCCACCCACGTCGGCCTCTGTCAGCTTCTGTTGTGTCTTCACAAAGTCTGTTTTAATATTTC
TGCTGTCTCTCCCATCTCTCCCCCATCCACACCGCGGTGGGCAAGTGCGTGCACCTGTGTGTG
TGGGTCCCTGAAAATGTGTGTGTGCATGTGTGTGTGTGTAAGTCACGCATGCACACATGTAT
GCTTCTGCACAGGTGTATGTGTGTGGTTGTATGCACGTGTGGCTCTGCTCTGCCATGTATGCA
CGTGTGTGGGGTGCATACATGTGTGTACACATGTGTGCATGTGTGTTGGGGATGGAATAATT
CTGTTCAGACATGAAGCTGCAGGGTTCCCGTGGGCCCCACCAGGCATCAGTTATGCTACTTG
GCACCTTTTCTTCTACCTGCCCTCCTTGGCATAGGTGTCCCCTAAGTCCACACACGTGCCCAC
ACATGTGCAGCCTCAGGCACATACAAACAAGGATATACACACGAGTGGGTGTTTATGTTGCC
TGTAATGGGGACACACGGCTGTTGAGAGTCTCTCCTGCAGTAGGGGACATGCTCCTGCCTAT
CAGGGTCCCCTCCCTCTCCCAGGCCCCCCAACCAAGGCTGTACCAGTTTCTCAGCCCTGGAA
GCGAGGCTGGGCCCCTGTTACAGGGAGAAGGCTGGGATGGTCAGGGACAGCCTGGGGGTCA
CAGCCCAACTCCGGGCCCTCCAGTGGGAGTGAAGCTCTCATCTGAGGAAGTCGAGAGCCCCA
CCCTGGGGCTCCTGCCCAGTCCTGCTTCAGGGGGCCAGGGAAGGGGCGCCTCCAGGCCTTAG
CCCACTCCTGCTGTGCAGGGGCCACTGAGCCATCCCCTTCACCCCGCTGGGCTCCAGCTGTG
AGGCCTCTTTCCCAGCGTCCTCTCTGGAGCAGCGGCCAACCCTGGCTGCGTGTGCGTGTGCGT
GTGTGTGCGGAACGCCCTCCTGTCTTGACTTCTGTGCTTTGTCTCCACATCTCTCTCACTTCAG
CTGTTTTTGTTCACCAGGATCTCTTGTCCTAAATTTTCCCTTTCCTTCCCTCTCTCTCTGGAAA
AACAAACAAAACAAAGCAAACACCCCAAGGACCCCAGGAGTCCTCGGCCGGCTCCTGACAA
TGCGTTTCCCGTTGCCCCGCCGCGGCCTGTCTGGTACCGGCTCCTGCTGCGGGCACCTCCCCT
GCACTCCACGCTCTGGGACGTGGCACCCGGGTCTCCGCAGTGGGGCCGCCCGCCACCCGCTC
CCGAGTCAAAAGCTTTGCTTGAAACACGGGAAGGACTTGGCAAATCCAAGGTCCAGCGAGG
CGGCGTCCACACCCTCTGTGGGAGAGCACCTGTTTCGACGGAGCCTCCCTGTTGCTTCCGTTT
CCGCCAGTTCCTGGCCAGCTCCTCATTTCCTGTGGCTTTACGATGCGTTTCGCCGGTGTCTCTT
TTCTTGTTTACTCTGCCAGGATAGATATGATTGTGGGTCCCCCGCCCCCTTCAACTCCCCGGC
ACAAGAAGTACCCCACCAAAGGACCCACGGCCCCTCCGAGAGAGTCACCCCAGTACTCACC
TAGGTTAGGATGCCAGCGCCTCTCCGACGTGTCTGTGGAATGGCCGGCCCCTTCGCTCCAGG
CCACATGACGGCAGCACCTTGTACCACCCTTTCCCCGGGACAGAGGGGACACTCGGGTTTTC
TCTGTTCCTGAAAAAGCCACAAGAGGGAGAAGGCCCTTCCTCCACCAGGGCCCCAGGCAGG
CTGTGCCCCTATCAGGACTGCCAGGGGCCCCACTGCTCCTCTCGACAAGTGCCCTGGGGAAG
CCGGGGGCTCTGTGGATGCCCTGTGAGATTTCCCTCTGGGGTTGGGAGGCCCCCGGGCAGCA
CCTCCAGGATCAGATGCCATCAGACCTGCACAGACATCCCTCTGGCCCTGCCCGTGCCCCCT
GGACAGGCCCCTCCTGGCTGAGGGCTCTCCAGCCTCCCCAGGCACTGACCTGCCCCTCAGCA
CCTGCCCACTGTGGGGGTGGGCCTGGCCACGTGCACGGCCCAGACCTGGGCCCTGGGTGGCT
CCGAGCAGGTGGAGCCGCATCTCTGTGTCTCTTCCCTCTCGTGGAAGATACAGACGGTCACA
GCATCTACCCCTTAAGCCACCGGGAAGAAGCGTGAGAAGAGGCCTGCCAGGTGCCCGGCAC
AGGCGTGCGCTCCAGCAGCTCCGGTGCTGGCTTTCAAGCTGCCAGCATCCTAATTAGTGTTTT
TGATAAGGGAGCTGGCGAGAGCCCCGGCTGATGGGCAGAACAGAGGTGCGGCCCAGCTCCA
CGTGAGCTGGCAGAAGCCAGTTCTTTAGTGATTAACTCAGACTTTCCAAGGCCTTCTTGCCTC
CCAGGGACTATTCAGATGACGGTGGTTCCCTGTCTTCTGGCAAGTCTGATTGGCTGAGGGAG
GGTAGAGCCCCTGGGGCGGAGGCCTGGAGGCTGAGCATAGACCCCTGGCCTTCCCTGACAG
CTCAGGGCAGACAGTTTCTGGGTACAGCCAATGCCAGTGTCTGGGCAAATGAAGCCCAGGCC
TGAGCTGGTGGGGACGGCGTCTGGGGTGAGTCTGACCCCTCCTCCCTCTGTGGTGGGGTTTCT
GCCTGTGAGGCCAAGTGTCTGTGCACTGGACATGTCAGCCCCGTGCTCCTCCCGTGAGAACG
GAGTTTCTGGGAGCAGGCAGGGCCGTGTCTGTTACTGCCCAACCCAGGGTAGCCCCGGACGG
AGTTTGCCGGGTTCCCTGCTCTGTGGTGTCCCCCATGCCCAAGAGTGCTGTGCCCTGGGGCTG
GTCCTCCGTCCTCCTGTGCCTTGCCGCAAGAGACAGCAGCTGGGCATCCACAGCTGTGAGCT
CGGACGGTCTTGTGGCTAGGAGACGTGCAGGTTCACACAGGGGGCCAGGGAGACAGTTGGA
GCGCTTGCCCCACCTCACGGGGCACTGAGCACTCAGGGATCAAGTCTCCTCCAGCCAGGGGC
CCAGGGCAGGAGGGGCACAGGGTCCTTGGAGGAGCCCCCAGCAGCAGCTCCAGGCTGAGCC
CGTGGCCAGGGCTCCTATTGGGACTGAGCAGGCGGGCGGCCCGTCATGTGGCAGGGGCGAA
GGTGCTGAGCATCTGCCTGGACTGTGTGGATGCCCCGAACACCAGGGACTCCCATCTCCTCC
ATCGCCACTGGCGTCTGCTCGGAGCCAGAGGGTCCCCTCCCGCCGTCCCCTCCCGCCCTCCTG
GCCCTATTGCATGAGTCCAGCATGAAGCCCCGCGGAGCATGAGGAGCTGCACTGTGGCCGAC
GCGAGCCTGAAACCCTCTCTGTTTCTCGCAACAGAAAGAAGATAACACCCCTGGCCCACCCT
CCCCCCTCCCATCAGAGGCAGGTAGGGCAGCCCAGTGGCAGGAAGTCCCGCCCCCATCAGA
GGCAGGTAGGGCAGCCCAGTGGCAGGAAGTCCCGCCCCCATCAGAGGCAGGTAGGGCAGCC
CAGTGGCAGGAAGTCCCGCCCCCATCAGAGGCAGGTAGGGCAGCCCAGTGGCAGGAAGTCC
CGCCCCCATCAGAGGCAGGTAGGGCAGCCCAGTGGCAGGAAGTCCCTCCCCCACCATCAGA
GGCAGGTAGGGCAGCCCAGTGGCAGGAAGTGAGGACTTGGCCCCGTCGGCTGGTGTGGGGC
AGTCTTCATGTACCAGGCCCCAGTCCAGGGGAGACTTGGGAGCTTGTGGCCTCTCCTGGGTG
GGTCCTGCTCAGCCACACCCCGTCTTGAGCTGCCCTTGTCCAGCTGGGCTTTTGTAGACTTAC
TGCAGGGTCTTTGTTTCCAAGTGAGTTCCCACGTAGCTGCAGGCCTCAGCGCACACCCACCGT
GGTGGCCCCTGCCAGCCCAGCAGTGGCCGTGACTCTGTGGATACCAGACTGGTCAGAAGCCC
CCGTCCCTGAGGGCAAAGGAGCCTCCCTCCCTTCTTATTGTCATGAGTGACCTGTGGCCTCAT
TGGAGATCAGCTCAGCTGAATTCTGCATCCTACACACACACATGCACACACGCAAACGTGCA
CACTCTTGCACACATGAACACATGCACACGCGTGCACACAGAAACTTGTGCAATGCACATGA
GGGCACACACATGCACATCGAGTGCACACACACGTGCACACACAGACCCTCGCAGCTTTGTC
TCCCCTCTTCTGGTTTGTGCGTTGGTTCCTGTGCTGCTCTGGGCCTTCTCTCCTTGTCCTGGTG
CATGGAGCCGCAGCTCTCACGCCTTTTCTGCTTCTGTTGTCCCCGCCGCAGACATTTAACTCC
GTCCCCATCAGACCCGGTGGGGCCGACGGCTTCAGTGGCACATTACGTAGGAGTAAAGCTCC
GGGGTGGGCACGGGTAGGGGGAGGGGCATCCTCTGGAGTGGGCTCCAGGCCCTGCAGTGGG
AGACCCTCCGTGGGCTGTGGGGCCAATAGGCCTTGGGCAGGGAAGAGGGTGAGCAGGTGGG
GCTGGCTGGAGGGGGCGGTATGGAGCCGGGAGCCCACGGAGAGGAGCCGGGCGACTCGGCC
CTGGTGGGAGCGTCCTTACTTTACTCCTCGCTAATGTGCCAGGCCCCTTGGGGACAGAGCGG
GCAGGAGCAGAACCTCACGGTGCCAGAAGACGGGCTGAGAACGGGGCGCACCCCGTCCATC
TGTCCTGGCACCCACTGCCCCCTGGTCGGCTCTGGGCTGAGCCTGCAGCCTAGACCCAGGGG
CACTGTGGCCTGGTCCAGGAGGAGAGGGCAGGGGCCCTGCTGCACCCACCCGAACTCATGCT
TTGGGGTCTCTGTTCCCGGTAGAGTGGACCAGATCGTGGGGCGGGGCCCAGCGATCACGGAC
AAGGACCGCACCAAGGGCCCGGCCGAGGCGGAGCTGCCCGAGGACCCCAGCATGATGGGAC
GGCTCGGGAAGGTGGAGAAGCAGGTGCGAGAGGCTGGGCGGGAGGGGTGCGGGGCTGGAG
GGCTGTGGTGCCTGCCGTCAACCTGTGGCAGTGTGGGCTTCAGGGGCTGGGTGCCACGGAGG
GCTCCTCAAGTCTAGGGTTTACATGGCCCTGTCACCACTGACAGCTGCTCTGTGCAGACCCCT
CACACCCCACCGTGAGCCTCCCACCCCCGGCCTGGGTGCCTTCCCTCCTGACAGTACCCAAG
GGCCAAAGTCCCCAGGAGTCCTGGGTGGGGGCTGCTGAGAGGTGGATCCCTCAGGGGTTCCT
TGTGGAACGTGCCGGAAGTTGTACCTGCCTGGCCTTCCTCCTCTCTGTCCTCCTTGCCCGACA
CCCTGGCCGCTGTCTTCTGGGGGTGCATTCTCAGGAACCACCGCAGAGGACCCTTGTTTTGGG
GCCTGCTTCTGAGAGCCGGGCCTGAGCAGTGAGTAAGGAACTGCCAGGTCCTGCCCTGTGGG
GCTTTCCTGAAGGGGGTTCCACGGTGGGCAGCAGGAGGGCCTGGCTCTGAACACACGCCCCA
ACCGTGGAGCTGGAGGCTTTGCCCCTAGGGGTCTGGGAAGGAGGAAGAGCTGCTCCTGGGC
CTCCTCTTCCCGCTCCTCCAGCTTCCAGGGAGCAGGGATCCCCCCTCCCTGTACCTCTCCAGG
GAGCAACCCAGGTCCCCCCACCTCCTGGGACCAGCCCAAGTCGACCGACTTCCTGGGAGTGG
TCCAGGTCCCCCCATCTCCTAGGACCAGCCTGGGTCCCCCCCATTTCCTGGGACTAGCCTGGG
TCCCCCATTTCCTGGGACTAGCCTGGGTCTCTCCTGTTTCCTGGAACCAGCCTGGGCCCCCCC
ATTTCCTGGGAGCAGCCCGGGTCCCCACATCTCCTGGGACTAGCCTGGGTCCCCCGTTTCCTG
GGACTAGCCTGGGTCTCTCCTGTTTCCTGGAACCAGCCTGGGCCCCCCCATTTCCTGGGAGCA
GCCTGGGTCCCCACATCTCCTGGGACGGGCCCTCGCACCCCAGCCCAGCAGCCCCTTTTGCA
GGTCTTGTCCATGGAGAAGAAGCTGGACTTCCTGGTGAATATCTACATGCAGCGGATGGGCA
TCCCCCCGACAGAGACCGAGGCCTACTTTGGGGCCAAAGAGCCGGAGCCGGCGCCGCCGTA
CCACAGCCCGGAAGACAGCCGGGAGCATGTCGACAGGCACGGCTGCATTGTCAAGATCGTG
CGCTCCAGCAGCTCCACGGGCCAGAAGAACTTCTCGGCGCCCCCGGCCGCGCCCCCTGTCCA
GTGTCCGCCCTCCACCTCCTGGCAGCCACAGAGCCACCCGCGCCAGGGCCACGGCACCTCCC
CCGTGGGGGACCACGGCTCCCTGGTGCGCATCCCGCCGCCGCCTGCCCACGAGCGGTCGCTG
TCCGCCTACGGCGGGGGCAACCGCGCCAGCATGGAGTTCCTGCGGCAGGAGGACACCCCGG
GCTGCAGGCCCCCCGAGGGGAACCTGCGGGACAGCGACACGTCCATCTCCATCCCGTCCGTG
GACCACGAGGAGCTGGAGCGTTCCTTCAGCGGCTTCAGCATCTCCCAGTCCAAGGAGAACCT
GGATGCTCTCAACAGCTGCTACGCGGCCGTGGCGCCTTGTGCCAAAGTCAGGCCCTACATTG
CGGAGGGAGAGTCAGACACCGACTCCGACCTCTGTACCCCGTGCGGGCCCCCGCCACGCTCG
GCCACCGGCGAGGGTCCCTTTGGTGACGTGGGCTGGGCCGGGCCCAGGAAGTGAGGCGGCG
CTGGGCCAGTGGACCCGCCCGCGGCCCTCCTCAGCACGGTGCCTCCGAGGTTTTGAGGCGGG
AACCCTCTGGGGCCCTTTTCTTACAGTAACTGAGTGTGGCGGGAAGGGTGGGCCCTGGAGGG
GCCCATGTGGGCTGAAGGATGGGGGCTCCTGGCAGTGACCTTTTACAAAAGTTATTTTCCAA
CAGGGGCTGGAGGGCTGGGCAGGGCCCTGTGGCTCCAGGAGCAGCGTGCAGGAGCAAGGCT
GCCCTGTCCACTCTGCTCAGGGCCGCGGCCGACATCAGCCCGGTGTGAGGAGGGGGGGGAG
TGATGACGGGGTGTTGCCAGCGTGGCAACAGGGGGGGGTTGTCTCAGCCGAGCCCAGGGG
AGGCACAAAGGGCAGGCCTGTTCCCTGAGGACCTGCGCAAAGGGCGGGCCTGTTTGGTGAG
GACCTGCGGCCTTGGGTCCCGGTGGGGTTTCCGGGCAGCTACAGGCGGGTGTGGCCGGCCGC
TGTGCGTGGCCTCTGCCTTCACACCTGACCTGCCCGGCGGGCTTTCCTGTTCCCCACCTCAGG
GGCGCCCAAATACAGAGCTATTGGTTGGCGTCTTCTCCCTGTACCTTCTGGGATCTGAGGGCT
CTTTCCATGGAAGCCAGCCCCGAGGTGGAGACCTTCGCCTGCAGCCGAGGAGCGGGTGGGG
CCTGGGAACCAAACTGGAGCCAGAGTGGACGTCCAGCCCTCTGGTCTTGGCCTCCAGAGGGA
GGGCCTGGCTCACGGTGGGGCCAGGGAGCCGGCTCCAAAGGGTCTTCAAAAAGGGGGTCCT
TGGGGGCTCCAGCTGCCTCGCCCTGGCCTTTCTGTGGGTGCGTGAGAGCCAGCAGCACCCCA
GCCTTGGAGACCGGGGGGGCAGGACCCCAAGTCCTCCCCTCTCTCCTGACTGCCCTGGCCGG
GTGCCGGCACTGCGAGACCCACCTGGTGAGCAGGCCTCACAGTTCTTAGCCAGGGCCCCACC
TCGCCTGTGTCCCACCAGTGCCCCGACAGACCTGGGGCAGGGCTGGGCCATGATGCAGCGGG
CCAGGATAGCCTCCACCGTCAGCACAGGGCCGCCCTCCCCGCCTTTCCGGAGGAAACCACTC
CCACCTCAGCCCAGCTGTGCGCCCTCCCTAGCTCTCCTGCCCCCTGGAGCTGATGGCCCCTTC
TCCACTGACCGATTCCTTAGCGGGGCCTCTTGGGGTCTCGGGCCTCGGGTGCACCGTCCCATG
CCCGTCCTGTTGTGGGCACCGTGGCCCTTGGGGCAGGCGGCTCTAATGCGGGAGCGAGTCCC
TAGCTCCAGACTTAAGAACCAGACCCCGGGAGCATCTGGCATTTGGCGTGACGGCGTCGCAG
GCGGGCCTGGGCTCCCTGGAGAGTGGCCTCCCTGGGAGTGAGCAGGGCTGGGGTCGTGGGC
GCAAATACTCCTGCAGAGCAAGTGCAGGGGAGTCCTGGGCCCGTTTCTCCTCCACCTGCGTT
TTCAGTGCACTTGGCTTGGCTGGGAGGTCCTGAGGCCCTGAGGCCAGCAGGGGAACCAGTCC
TGAGGGAGAGGACTTTGAAAGCAGCATTTGAGGGTCGTACGCCCCTGGCTGGTGGGGGTCCT
GGCGCTCAGGGTGTTCGGGGAGCCATGTCTGGCGTCCATTGTGGGGAGCTGCTGCCCTGGCC
TCTCTGCCTACCCCCAGCCCGGCCAGGGCACTCCCAGGCCCTGTCGCCATTGAGGTGCCTCC
GCTGGGCTGTCTCCTCACCCCTCCCTGTGCTGGAGCCTGTCCCAAAAAGGTGCCAACTGGGA
GGCCTCGGAAGCCACTGTCCAGGCTCCCACTGCCTGTCTGCTCTGTTCCCAAAGGCAGCGTG
TGTGGCCTCGGGCCCTGCGGTGGCATGAAGCATCCCTTCTGGTGTGGGCATCGCTACGTGTTT
TGGGGGCAGCGTTTCACGGCGGTGCCCTTGCTGTCTCCCTTGGGCTGGCTCGAGCCTGGGGTC
CATGTCCCTTTGCCGTCCCGTCATGGGGCAGGGAATCCATAGCGGGGCCCACAGGCAGGGGT
ATGAGTGCGTCCCACCCAACGCAGCACCAGCCCCGGCCACCGCTCCCCGTGTCCCCAGTTCC
GTCTCAGCTACCTGGACTCCAGGACCCTGGAGAAGGGAGACCTGGCAGTGGAGGGAGGCTG
TGCTGTGTGTCCCCCTGCAGGTGTGACCCCGCCTGCTCTTTCCTCCCCCGCCAGGTGTGGCCC
CGCCTGCTCTTTCCTCCCCCACCAGTATGGCCCCACCTGCTCTTTCCTCCCCCCCCAAGGTGT
GGCCCCACCTGTTCTTTCCTCCCCTGCCGAGGTGTGACCCCACCTGCTCTTTCCTCCCTCCCAG
TATGGCCCCACCTGCTCTTTCCTCCCCCGAGGTGAGGCCCCGCCTGCTCTTTCCTCCCATGGG
GCCGCTGAGGCATGAGCACCTGGGCACAGGTTGGGGCTCTGCAGGATGAGGAAGACAGGCC
AATCCCTTCCCTCCCAGAAGCTGGCCGCCCAGCAGGAGGGACTGAGGCCAGACTCATGTCCA
GCAAGGAACGTGTGGTGTGTCCCCTGGGAAGTCTCTGGGCCCTGGGAAGAGGGAAGGTGCA
CGTCCTGGGATGGTTGCGGGGCCCTGTTTTGGGAGACAAAGGGGTAGAGGGTCTGTCTTGGG
CCCCCCCAGACTCTAGCCTGAGCAGTGCAGCCACCTACTGCCCCACCTCAGAGAAGTGCAGC
GGGAAGGAGGCTGGAGGTGGTGCGGCGCTGCCTCGGGTGTCTGCGTGAATGAGCGTGGCCA
AGGACCAGTGCCACCTCATGGCAAAGAGCTCCCGCAGTGTTTGTTAGAGTGCACATCCCTAC
GTGCCCACTGGCACACACACGTGCTCACATACATGTCCGCATACAGGCGTACACATGCACGC
TTGCACACATGCACACAGACCACATAGCACACATGTGCACTGACCACACCTGTATAGACCAT
GCACAGTACACATACGTGCATACACATGCCTGCATACAGGCATACACATGCACGCTTACATG
TACACGTGCACAGATCACACACATGCACACACGTGTAGCTCACACACAGTATACACATACAC
AAGTGCACAGACCACACACAGCACTAACACATGCACACACAAAGTGCATAGGCCACACAGC
ACATGCACACAGGTGCACAGACCACACAGCACACACAAGTGCACAGAGCACACTGCACACA
TGCACACACACACGCGTGCATGCACACTCCTCGCACTTCCAGCCTTGGAGCCCTTCTGTCTCT
GGTCTTTCTCTTTGACCCTGCTGAGTGTAAGCTGCCTGGGGAGGGGCTACAAGGAGTAATTG
TGGCTTTAGGGGTCGTGGTGATGCTGGAATGTCAAGCGCCGTCGTGGGGTATCCGACTGTCC
GGGCTCCTGGTCCGCAGTGGCAGAGCGCCAGGCAGAGCCCAATCAGGGTCTCGTGCTGCCCT
TCCCTCCCACAGCCTGGCAGCCATCCAGAGGAGGGGCTCTACCAGATGCCAAGGTGCCCCGG
TGTCTGTATGGGTGTCCGGTTGGGTCCTGTGTTTGGTCTGCCCTGGAGGTGCTGGGCCCTCCT
GGGATGGGTGGCTCAGCCTCGAATCCCAGGCCCCAGCCCAGGCAGGTGCTGCTGCCTGTTGT
GGTTTCCTGGCCCAGCTTCTCCTTCTCCCTCTGCATAAAATCACAGTCCGTGAGTCTTCCAGC
TGCCACCACGGCTGGGACACGCTGGGGGAGGGCTCCTCCCATGCCTCCTGCACACAGCCGTC
TGAGCAGGGCAGGTGCCCAACACCCCCCACCGGAGGACACGCTGCCCCTCAGCGATGCCCCT
ACCTTTTGGGGGGCCTCGTCTCAAGCCCCCCCTTGGAGGCTGAAATCACCCCAGGCACTGTG
AGGGCTTCTCCAGGGGGCACCCTTTGAGCTGTGGGTCTGATCACCCCAAGTCCCGCCCGGAG
GAGAGGCACAGCCAGGGCGTGTGGTTTAATGTTTGCCCCCTTCGGGGCTGGAGGTCTCAGTG
TTTCTAGATTCCAGACCCTGCTGCCAGAGAGACCTGCTGCCGGAGAGAAGGGGAGGAGGAC
TCCAGCTGGGCTCGGTCCCCCACAGTCAGGGACCCCCATAAAGGACACCCCCTTCTCTCTAG
AAAGAGCTGGGCTCTCAGCTATTTCTAGTTGCTTCCCAGAAGCCGAGGAGCAGAAGGAGCTG
TGAGAGCTTTGCAGAAACGCCCTTGTCCCCGCCCTCCTGAGCTATGAATGCCGTACAGAGCA
GAGGCTGGGGCATTGGCAAGATCACAGGTTGATGCTGCACAGCCCCATTGACACAAACCCTC
AAAGCAGACGTGAGAGGGACGGTTCACAAAGCTCGGACCTGCCGTGGAGGGTGCCCGGCAG
ACGTGGCGTGAGAGGGACGGCTCACGAGGCTTGGACCTGCTGTGGAGGGTGCCCAGCAGAC
GTGGTGTGAGAGGAACGGCTCACGAGACTTGGACCTGGTGGAGGGTGCCCAGCAGACGTGG
TGTGAGAGGGACGGCTCACAGGGCTTGGACCTGCCATGGAGGGTGCCCGGCAGACGTGGTG
TGAGAGGGATGGTTCACAGGGCTTGGACCTGCCATGGAGGGTGCCCGGCAGATGTGGTGGG
AGAGAGATGGCTCATGAGGCTTGGACCTGCCGTGGAGGGTGCCCAGCAGACGTGGTATGAG
AGGGATGGCTCACGAGGCTTGGACCTGGTGGAGGGTGCCCGGCAGACGTGTGAGAGGGACG
GTTCACAAGGCTTGGACCTGCCATGGAGGGTGCCCAGCAGACGTGGTGTGAGAGGGACAGC
TCACGAGGCTTGGACCTGCTGTGGAGGGTGCCCAGCAGGGGGCTGAGCTCTGAGGGGTGGG
TGCTCAGTGCACGGGTGCCCCCAGTGTCCTCTGATCCTGTCCGGTGCCTCCCCCAACCCCCAC
ACCCATGCAGAACTCCCAGGTCACATGCACGTATGTCCAGGGCATGGGGGTGGCGTGAAGA
GGCCTGGTCAGGGCCTTTAGGGGCTGCAGGACGGAATGGCCGCCTGGGGAGCCTGTGTGGCT
GTGCCGGGCAGCCATCCTGCATTCCCACCCAGCGCGCAGTCTCCACCTCGGCCCCAGCAAAG
CGCTAAGCAGCCGGAGAGACAGCCAGGGCGGCTTCCTGAAGGATGTGGGATGGTGGACTCC
GGGGTCGAGGGAATACGCAGGTTCCTGTCCCTCCGGGAGACCTAGAGAAGCTGCACACCCA
GGAGCTTTCCATGACCCGGGAGCATGAGTGAATGGGGGTTCCAGTTTGCTGAACTTTGCCGT
CTTGTAAGGGTGGGGGCTGACGGCCGACCCTGGGAGGAGGTGACATCGCCGGGGGAGGTTG
TGGGCAACGGTGGAGGAGGAGAGACGGGAGGGGACCATTTGGGATGGAGGGGCCTCTTCAG
AGTTTTAAAAGGCGTTTGTGGGGTGGAGTTGAGTGTGCTCTGGGCTTGGACACTTGCCGTGG
TGCCCCTGGCTGGCCGAGGAGACTGGCTCTGGCCAGGGGCCCCGTCCTGAGAGGTCCTCAGC
GTCTGACTCTCGGCCAGGCGCCAGCAAGGAGGGGCCGGTCCCCGGGGCTACCAGGCAGGCA
CGTGCACATCGCCATCGCCACACGCCAACTCCGCCTGGGTTTTACAAAGTCGTTGCCTTAATG
CATGTGGACAGGAACTCCCTGAGGTCGCCCCATGCCCCCTGGCTGTGCCAGGTACGGACGCC
CTGGACCCTGCGAACAGGTGGGGGGGGCGAGGGGCCCAAGGGACGGGCTCCAGAGACACGC
GCAGGGCAGGAGGGGTCTCACGGAGGGGTCTCGCACTGAGGCGCCCAGAGCTGGTGGTCCC
GCTGGACGCCATCCCTCTGCCCGGGATCCACACGGCCCACGTGTGCCCGCCATGCCCGCGCC
CCACGCCATTGCAGTCTGCCATCCTCTGGCCGTGACGGTGGCTGCAGCTTCCCCATTTGCGCC
GTTGCCTCTGGCTGTCTGCACTTTTGTTCATGCTCCAAAGAACATTTCATAATGCCTTCAGTA
CCGACGTACACTTCTGACCATTTTGTATGTGTCCTTGTGCCGTAGTGACCAGGCCTTTTTTTGG
TGGATGTGTTACCCCGCACACTTCAATCTCAACTTTGTGCACCGTCCATTTTCTAGGGATAGA
CGCCCAGGGAATGAACTCTAGTTTTCTAACAGATTAGCTGAGATATTAACTTACTCACACGG
ACAGGTTGATGCCAGAGCCGTAAGAATGCGCCAGTGCGGGTTTGCGGGGGACTTCGGGTGTG
GGGTCCTGCGGCCGCGATGGCCGTGGAAGGTTCTGGGGATCCCTGCTGCCACGGGGACGAGT
TCGGACGCCAGGTGGACCTGTGCACTCAGTAAAACGCAGTGATTCAACCTGGA
NCBI Reference Sequence NG_009004.2 is SEQ ID NO: 3044.

In various embodiments, a KCNQ2 pre-mRNA transcript comprises a sequence provided as SEQ ID NO: 3045. NCBI Reference Sequence NC_000020.11 Reference GRCh38.p13 Primary Assembly is SEQ ID NO: 3045.

In various embodiments, the KCNQ2 transcript shares between 90-100% identity with any one of SEQ ID NO: 3032-3043 or shares between 90-100% identity to a KCNQ2 pre-mRNA transcript comprising SEQ ID NO: 3044 or SEQ ID NO: 3045. In various embodiments, the KCNQ2 transcript shares at least 90%, 91%, 92%, 93%, 94%, 95%, 96%, 97%, 98%, or 99% identity with SEQ ID NO: 3032-3043 or shares at least 90%, 91%, 92%, 93%, 94%, 95%, 96%, 97%, 98%, or 99% identity with a KCNQ2 pre-mRNA transcript transcribed from SEQ ID NO: 3044 or SEQ ID NO: 3045.

In various embodiments, KCNQ2 is indirectly regulated by TDP43, and as such is encompassed by the phrase “regulated by TDP43”.

### KCNQ2 Oligonucleotides Targeting Regions of the KCNQ2 Transcript

In various embodiments, KCNQ2 AON disclosed herein are complementary to specific regions of KCNQ2 transcripts (for example, a KCNQ2 transcript) comprising a sequence that shares at least 90% (e.g., 90%, 91%, 92%, 93%, 94%, 95%, 96%, 97%, 98%, 99%, or 100%) identity to any one of SEQ ID NOs: 3032-3045. In some embodiments, a KCNQ2 AON comprises a sequence that is complementary to a specific region of the KCNQ2 transcript comprising a sequence that shares at least 90% (e.g., 90%, 91%, 92%, 93%, 94%, 95%, 96%, 97%, 98%, 99%, or 100%) identity to any one of SEQ ID NOs: 3032-3045. In some embodiments, a KCNQ2 AON comprises a sequence that is at least 85% complementary to a specific region of the KCNQ2 transcript. In some embodiments, a KCNQ2 AON comprises a sequence that is at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% complementary to a specific region of the KCNQ2 transcript. In some embodiments, a KCNQ2 AON comprises a sequence that is 90 to 99% complementary to a specific region of the KCNQ2 transcript. In some embodiments, a KCNQ2 AON comprises a sequence that is 90 to 95% complementary to a specific region of the KCNQ2 transcript. In some embodiments, a KCNQ2 AON comprises a sequence that is 95 to 99% complementary to a specific region of the KCNQ2 transcript. In various embodiments, the KCNQ2 transcript is a pre-mRNA transcript and the specific region is intron 4 of the KCNQ2 pre-mRNA transcript. In various embodiments, the KCNQ2 transcript is a pre-mRNA transcript and the specific region is intron 5 of the KCNQ2 pre-mRNA transcript. In various embodiments, the specific region is exon 5 of the KCNQ2 pre-mRNA transcript.

In some embodiments, the KCNQ2 AON (e.g., KCNQ2 AON) has a segment that has, at most, 7 linked nucleosides. In some embodiments, the KCNQ2 AON has a segment that has, at most, 6, 5, 4, 3, or 2 linked nucleosides. The segments of the KCNQ2 AON may be separated from other segments of the KCNQ2 AON through a spacer. The segment of the KCNQ2 AON is complementary to a specific region of the KCNQ2 transcript (for example, a KCNQ2 transcript) comprising a sequence that shares at least 90% (e.g., 90%, 91%, 92%, 93%, 94%, 95%, 96%, 97%, 98%, 99%, or 100%) identity to any one of SEQ ID NOs: 3032-3045.

### KCNQ2 Oligonucleotide Variants

In various embodiments, KCNQ2 AONs include different variants, hereafter referred to as KCNQ2 AON variants. A KCNQ2 AON variant may be an oligonucleotide sequence of 5 to 100 nucleobases in length, for example, 10 to 40 nucleobases in length, for example, 14 to 40 nucleobases in length, 10 to 30 nucleobases in length, for example, 14 to 30 nucleobases in length, for example, 16 to 28 nucleobases in length, for example, 19 to 23 nucleobases in length, for example, 21 to 23 nucleobases in length, for example, or 18, 19, 20, 21, 22, 23, 24, 25, 26, 27, 28, 29, or 30 nucleobases in length. A KCNQ2 AON variant may be an oligonucleotide sequence complementary to a portion of a KCNQ2 pre-mRNA sequence or a KCNQ2 gene sequence. Example KCNQ2 AON variants include a nucleobase sequence selected from any one of SEQ ID NOs: SEQ ID NOs: 4402-4468, SEQ ID NOs: 4469-4476, or SEQ ID NOs: 4477-4530.

In various embodiments, a KCNQ2 AON variant represents a modified version of a corresponding KCNQ2 parent oligonucleotide that includes a nucleobase sequence selected from any one of SEQ ID NOs: 1676-1851 and SEQ ID NOs: 2028-2529. In some embodiments, a KCNQ2 AON variant includes a nucleobase sequence that represents a shortened version of a nucleobase sequence of a KCNQ2 AON selected from any one of SEQ ID NOs: 1676-1851 and SEQ ID NOs: 2028-2529. As one example, if a KCNQ2 parent oligonucleotide includes a 25mer (e.g., 25 nucleotide bases in length) a variant (e.g., a KCNQ2 variant) may include a shorter version (e.g., 15mer, 17mer, 19mer, 21mer, or 23mer) of the 25mer KCNQ2 parent oligonucleotide. In one embodiment, a nucleobase sequence of a KCNQ2 AON variant differs from a corresponding nucleobase sequence of a KCNQ2 parent oligonucleotide in that 1, 2, 3, 4, 5, or 6 nucleotide bases are removed from one or both of the 3′ and 5′ ends of the nucleobase sequence of the KCNQ2 parent oligonucleotide. In one embodiment, the corresponding KCNQ2 AON variant may include a 23mer where two nucleotide bases were removed from one of the 3′ or 5′ end of a 25mer included in the KCNQ2 parent oligonucleotide. In one embodiment, the corresponding KCNQ2 AON variant may include a 23mer where one nucleotide base is removed from each of the 3′ and 5′ ends of the 25mer included in the KCNQ2 parent oligonucleotide. In one embodiment, the corresponding KCNQ2 AON variant may include a 21mer where two nucleotide bases are removed from each of the 3′ and 5′ ends of the 25mer included in the KCNQ2 parent oligonucleotide. In one embodiment, the corresponding KCNQ2 AON variant may include a 21mer where four nucleotide bases are removed from either the 3′ or 5′ end of the 25mer included in the KCNQ2 parent oligonucleotide. In one embodiment, the corresponding KCNQ2 AON variant may include a 19mer where three nucleotide bases are removed from each of the 3′ and 5′ ends of the 25mer included in the KCNQ2 parent oligonucleotide. In one embodiment, the corresponding KCNQ2 AON variant may include a 19mer where six nucleotide bases are removed from either the 3′ or 5′ end of the 25mer included in the KCNQ2 parent oligonucleotide.

Example sequences of KCNQ2 AON variants are shown below in Tables 8A and 8B.

TABLE 8A
KCNQ2 Oligonucleotide Variant Sequences complementary to a sequence of a
KCNQ2 transcript

| SEQ ID | AON Sequence* | SEQ ID | Target Sequence |
|---|---|---|---|
| NO: | (5′→3′) | NO: | (5′→3′) |
| 3046 | CGCCAGCCTTGGGGCCGTGACTC | 3222 | GAGTCACGGCCCCAAGGCTGGCG |
| 3047 | CCGCCAGCCTTGGGGCCGTGACT | 3223 | AGTCACGGCCCCAAGGCTGGCGG |
| 3048 | ACCGCCAGCCTTGGGGCCGTGAC | 3224 | GTCACGGCCCCAAGGCTGGCGGT |
| 3049 | CACCGCCAGCCTTGGGGCCGTGA | 3225 | TCACGGCCCCAAGGCTGGCGGTG |
| 3050 | CCACCGCCAGCCTTGGGGCCGTG | 3226 | CACGGCCCCAAGGCTGGCGGTGG |
| 3051 | CCCACCGCCAGCCTTGGGGCCGT | 3227 | ACGGCCCCAAGGCTGGCGGTGGG |
| 3052 | GCCCACCGCCAGCCTTGGGGCCG | 3228 | CGGCCCCAAGGCTGGCGGTGGGC |
| 3053 | CGCCCACCGCCAGCCTTGGGGCC | 3229 | GGCCCCAAGGCTGGCGGTGGGCG |
| 3054 | GCGCCCACCGCCAGCCTTGGGGC | 3230 | GCCCCAAGGCTGGCGGTGGGCGC |
| 3055 | GGCGCCCACCGCCAGCCTTGGGG | 3231 | CCCCAAGGCTGGCGGTGGGCGCC |
| 3056 | GGGCGCCCACCGCCAGCCTTGGG | 3232 | CCCAAGGCTGGCGGTGGGCGCCC |
| 3057 | GGGGCGCCCACCGCCAGCCTTGG | 3233 | CCAAGGCTGGCGGTGGGCGCCCC |
| 3058 | GGGGGCGCCCACCGCCAGCCTTG | 3234 | CAAGGCTGGCGGTGGGCGCCCCC |
| 3059 | TGGGGGCGCCCACCGCCAGCCTT | 3235 | AAGGCTGGCGGTGGGCGCCCCCA |
| 3060 | CTGGGGGCGCCCACCGCCAGCCT | 3236 | AGGCTGGCGGTGGGCGCCCCCAG |
| 3061 | GCTGGGGGCGCCCACCGCCAGCC | 3237 | GGCTGGCGGTGGGCGCCCCCAGC |
| 3062 | GGCTGGGGGCGCCCACCGCCAGC | 3238 | GCTGGCGGTGGGCGCCCCCAGCC |
| 3063 | TGGCTGGGGGCGCCCACCGCCAG | 3239 | CTGGCGGTGGGCGCCCCCAGCCA |
| 3064 | CTGGCTGGGGGCGCCCACCGCCA | 3240 | TGGCGGTGGGCGCCCCCAGCCAG |
| 3065 | GCTGGCTGGGGGCGCCCACCGCC | 3241 | GGCGGTGGGCGCCCCCAGCCAGC |
| 3066 | CGCTGGCTGGGGGCGCCCACCGC | 3242 | GCGGTGGGCGCCCCCAGCCAGCG |
| 3067 | TCGCTGGCTGGGGGCGCCCACCG | 3243 | CGGTGGGCGCCCCCAGCCAGCGA |
| 3068 | CTCGCTGGCTGGGGGCGCCCACC | 3244 | GGTGGGCGCCCCCAGCCAGCGAG |
| 3069 | TCTCGCTGGCTGGGGGCGCCCAC | 3245 | GTGGGCGCCCCCAGCCAGCGAGA |
| 3070 | CTCTCGCTGGCTGGGGGCGCCCA | 3246 | TGGGCGCCCCCAGCCAGCGAGAG |
| 3071 | ACTCTCGCTGGCTGGGGGCGCCC | 3247 | GGGCGCCCCCAGCCAGCGAGAGT |
| 3072 | GACTCTCGCTGGCTGGGGGCGCC | 3248 | GGCGCCCCCAGCCAGCGAGAGTC |
| 3073 | GGACTCTCGCTGGCTGGGGGCGC | 3249 | GCGCCCCCAGCCAGCGAGAGTCC |
| 3074 | AGGACTCTCGCTGGCTGGGGGCG | 3250 | CGCCCCCAGCCAGCGAGAGTCCT |
| 3075 | CAGGACTCTCGCTGGCTGGGGGC | 3251 | GCCCCCAGCCAGCGAGAGTCCTG |
| 3076 | CCAGGACTCTCGCTGGCTGGGGG | 3252 | CCCCCAGCCAGCGAGAGTCCTGG |
| 3077 | GCCAGGACTCTCGCTGGCTGGGG | 3253 | CCCCAGCCAGCGAGAGTCCTGGC |
| 3078 | GGCCAGGACTCTCGCTGGCTGGG | 3254 | CCCAGCCAGCGAGAGTCCTGGCC |
| 3079 | GGGCCAGGACTCTCGCTGGCTGG | 3255 | CCAGCCAGCGAGAGTCCTGGCCC |
| 3080 | TGGGCCAGGACTCTCGCTGGCTG | 3256 | CAGCCAGCGAGAGTCCTGGCCCA |
| 3081 | CTGGGCCAGGACTCTCGCTGGCT | 3257 | AGCCAGCGAGAGTCCTGGCCCAG |
| 3082 | TCTGGGCCAGGACTCTCGCTGGC | 3258 | GCCAGCGAGAGTCCTGGCCCAGA |
| 3083 | GTCTGGGCCAGGACTCTCGCTGG | 3259 | CCAGCGAGAGTCCTGGCCCAGAC |
| 3084 | GGTCTGGGCCAGGACTCTCGCTG | 3260 | CAGCGAGAGTCCTGGCCCAGACC |
| 3085 | CGGTCTGGGCCAGGACTCTCGCT | 3261 | AGCGAGAGTCCTGGCCCAGACCG |
| 3086 | CCGGTCTGGGCCAGGACTCTCGC | 3262 | GCGAGAGTCCTGGCCCAGACCGG |
| 3087 | CCCGGTCTGGGCCAGGACTCTCG | 3263 | CGAGAGTCCTGGCCCAGACCGGG |
| 3088 | GCCCGGTCTGGGCCAGGACTCTC | 3264 | GAGAGTCCTGGCCCAGACCGGGC |
| 3089 | GGCCCGGTCTGGGCCAGGACTCT | 3265 | AGAGTCCTGGCCCAGACCGGGCC |
| 3090 | GGGCCCGGTCTGGGCCAGGACTC | 3266 | GAGTCCTGGCCCAGACCGGGCCC |
| 3091 | GGGGCCCGGTCTGGGCCAGGACT | 3267 | AGTCCTGGCCCAGACCGGGCCCC |
| 3092 | TGGGGCCCGGTCTGGGCCAGGAC | 3268 | GTCCTGGCCCAGACCGGGCCCCA |
| 3093 | GTGGGGCCCGGTCTGGGCCAGGA | 3269 | TCCTGGCCCAGACCGGGCCCCAC |
| 3094 | GGTGGGGCCCGGTCTGGGCCAGG | 3270 | CCTGGCCCAGACCGGGCCCCACC |
| 3095 | GGGTGGGGCCCGGTCTGGGCCAG | 3271 | CTGGCCCAGACCGGGCCCCACCC |
| 3096 | GGGGTGGGGCCCGGTCTGGGCCA | 3272 | TGGCCCAGACCGGGCCCCACCCC |
| 3097 | AGGGGTGGGGCCCGGTCTGGGCC | 3273 | GGCCCAGACCGGGCCCCACCCCT |
| 3098 | CAGGGGTGGGGCCCGGTCTGGGC | 3274 | GCCCAGACCGGGCCCCACCCCTG |
| 3099 | GCAGGGGTGGGGCCCGGTCTGGG | 3275 | CCCAGACCGGGCCCCACCCCTGC |
| 3100 | GGCAGGGGTGGGGCCCGGTCTGG | 3276 | CCAGACCGGGCCCCACCCCTGCC |
| 3101 | AGGCAGGGGTGGGGCCCGGTCTG | 3277 | CAGACCGGGCCCCACCCCTGCCT |
| 3102 | CAGGCAGGGGTGGGGCCCGGTCT | 3278 | AGACCGGGCCCCACCCCTGCCTG |
| 3103 | CCAGGCAGGGGTGGGGCCCGGTC | 3279 | GACCGGGCCCCACCCCTGCCTGG |
| 3104 | CCCAGGCAGGGGTGGGGCCCGGT | 3280 | ACCGGGCCCCACCCCTGCCTGGG |
| 3105 | CCCCAGGCAGGGGTGGGGCCCGG | 3281 | CCGGGCCCCACCCCTGCCTGGGG |
| 3106 | ACCCCAGGCAGGGGTGGGGCCCG | 3282 | CGGGCCCCACCCCTGCCTGGGGT |
| 3107 | AACCCCAGGCAGGGGTGGGGCCC | 3283 | GGGCCCCACCCCTGCCTGGGGTT |
| 3108 | AAACCCCAGGCAGGGGTGGGGCC | 3284 | GGCCCCACCCCTGCCTGGGGTTT |
| 3109 | CAAACCCCAGGCAGGGGTGGGGC | 3285 | GCCCCACCCCTGCCTGGGGTTTG |
| 3110 | GCAAACCCCAGGCAGGGGGGGG | 3286 | CCCCACCCCTGCCTGGGGTTTGC |
| 3111 | AGCAAACCCCAGGCAGGGGTGGG | 3287 | CCCACCCCTGCCTGGGGTTTGCT |
| 3112 | AAGCAAACCCCAGGCAGGGGTGG | 3288 | CCACCCCTGCCTGGGGTTTGCTT |
| 3113 | GAAGCAAACCCCAGGCAGGGGTG | 3289 | CACCCCTGCCTGGGGTTTGCTTC |
| 3114 | TGAAGCAAACCCCAGGCAGGGGT | 3290 | ACCCCTGCCTGGGGTTTGCTTCA |
| 3115 | TTGAAGCAAACCCCAGGCAGGGG | 3291 | CCCCTGCCTGGGGTTTGCTTCAA |
| 3116 | CTTGAAGCAAACCCCAGGCAGGG | 3292 | CCCTGCCTGGGGTTTGCTTCAAG |
| 3117 | TCTTGAAGCAAACCCCAGGCAGG | 3293 | CCTGCCTGGGGTTTGCTTCAAGA |
| 3118 | CTCTTGAAGCAAACCCCAGGCAG | 3294 | CTGCCTGGGGTTTGCTTCAAGAG |
| 3119 | GCTCTTGAAGCAAACCCCAGGCA | 3295 | TGCCTGGGGTTTGCTTCAAGAGC |
| 3120 | GGCTCTTGAAGCAAACCCCAGGC | 3296 | GCCTGGGGTTTGCTTCAAGAGCC |
| 3121 | GGGCTCTTGAAGCAAACCCCAGG | 3297 | CCTGGGGTTTGCTTCAAGAGCCC |
| 3122 | AGGGCTCTTGAAGCAAACCCCAG | 3298 | CTGGGGTTTGCTTCAAGAGCCCT |
| 3123 | CAGGGCTCTTGAAGCAAACCCCA | 3299 | TGGGGTTTGCTTCAAGAGCCCTG |
| 3124 | CCAGGGCTCTTGAAGCAAACCCC | 3300 | GGGGTTTGCTTCAAGAGCCCTGG |
| 3125 | CCCAGGGCTCTTGAAGCAAACCC | 3301 | GGGTTTGCTTCAAGAGCCCTGGG |
| 3126 | ACCCAGGGCTCTTGAAGCAAACC | 3302 | GGTTTGCTTCAAGAGCCCTGGGT |
| 3127 | CACCCAGGGCTCTTGAAGCAAAC | 3303 | GTTTGCTTCAAGAGCCCTGGGTG |
| 3128 | CCACCCAGGGCTCTTGAAGCAAA | 3304 | TTTGCTTCAAGAGCCCTGGGTGG |
| 3129 | TCCACCCAGGGCTCTTGAAGCAA | 3305 | TTGCTTCAAGAGCCCTGGGTGGA |
| 3130 | CTCCACCCAGGGCTCTTGAAGCA | 3306 | TGCTTCAAGAGCCCTGGGTGGAG |
| 3131 | CCTCCACCCAGGGCTCTTGAAGC | 3307 | GCTTCAAGAGCCCTGGGTGGAGG |
| 3132 | CCCTCCACCCAGGGCTCTTGAAG | 3308 | CTTCAAGAGCCCTGGGTGGAGGG |
| 3133 | TCCCTCCACCCAGGGCTCTTGAA | 3309 | TTCAAGAGCCCTGGGTGGAGGGA |
| 3134 | ATCCCTCCACCCAGGGCTCTTGA | 3310 | TCAAGAGCCCTGGGTGGAGGGAT |
| 3135 | CATCCCTCCACCCAGGGCTCTTG | 3311 | CAAGAGCCCTGGGTGGAGGGATG |
| 3136 | CCATCCCTCCACCCAGGGCTCTT | 3312 | AAGAGCCCTGGGTGGAGGGATGG |
| 3137 | TCCATCCCTCCACCCAGGGCTCT | 3313 | AGAGCCCTGGGTGGAGGGATGGA |
| 3138 | CTCCATCCCTCCACCCAGGGCTC | 3314 | GAGCCCTGGGTGGAGGGATGGAG |
| 3139 | ACTCCATCCCTCCACCCAGGGCT | 3315 | AGCCCTGGGTGGAGGGATGGAGT |
| 3140 | GACTCCATCCCTCCACCCAGGGC | 3316 | GCCCTGGGTGGAGGGATGGAGTC |
| 3141 | TGACTCCATCCCTCCACCCAGGG | 3317 | CCCTGGGTGGAGGGATGGAGTCA |
| 3142 | CTGACTCCATCCCTCCACCCAGG | 3318 | CCTGGGTGGAGGGATGGAGTCAG |
| 3143 | ACTGACTCCATCCCTCCACCCAG | 3319 | CTGGGTGGAGGGATGGAGTCAGT |
| 3144 | CACTGACTCCATCCCTCCACCCA | 3320 | TGGGTGGAGGGATGGAGTCAGTG |
| 3145 | CCACTGACTCCATCCCTCCACCC | 3321 | GGGTGGAGGGATGGAGTCAGTGG |
| 3146 | ACCACTGACTCCATCCCTCCACC | 3322 | GGTGGAGGGATGGAGTCAGTGGT |
| 3147 | CACCACTGACTCCATCCCTCCAC | 3323 | GTGGAGGGATGGAGTCAGTGGTG |
| 3148 | CCACCACTGACTCCATCCCTCCA | 3324 | TGGAGGGATGGAGTCAGTGGTGG |
| 3149 | GCCACCACTGACTCCATCCCTCC | 3325 | GGAGGGATGGAGTCAGTGGTGGC |
| 3150 | AGCCACCACTGACTCCATCCCTC | 3326 | GAGGGATGGAGTCAGTGGTGGCT |
| 3151 | GAGCCACCACTGACTCCATCCCT | 3327 | AGGGATGGAGTCAGTGGTGGCTC |
| 3152 | AGAGCCACCACTGACTCCATCCC | 3328 | GGGATGGAGTCAGTGGTGGCTCT |
| 3153 | CAGAGCCACCACTGACTCCATCC | 3329 | GGATGGAGTCAGTGGTGGCTCTG |
| 3154 | CCAGAGCCACCACTGACTCCATC | 3330 | GATGGAGTCAGTGGTGGCTCTGG |
| 3155 | GCCAGAGCCACCACTGACTCCAT | 3331 | ATGGAGTCAGTGGTGGCTCTGGC |
| 3156 | AGCCAGAGCCACCACTGACTCCA | 3332 | TGGAGTCAGTGGTGGCTCTGGCT |
| 3157 | CAGCCAGAGCCACCACTGACTCC | 3333 | GGAGTCAGTGGTGGCTCTGGCTG |
| 3158 | CCAGCCAGAGCCACCACTGACTC | 3334 | GAGTCAGTGGTGGCTCTGGCTGG |
| 3159 | TCCAGCCAGAGCCACCACTGACT | 3335 | AGTCAGTGGTGGCTCTGGCTGGA |
| 3160 | CTCCAGCCAGAGCCACCACTGAC | 3336 | GTCAGTGGTGGCTCTGGCTGGAG |
| 3161 | GCTCCAGCCAGAGCCACCACTGA | 3337 | TCAGTGGTGGCTCTGGCTGGAGC |
| 3162 | GGCTCCAGCCAGAGCCACCACTG | 3338 | CAGTGGTGGCTCTGGCTGGAGCC |
| 3163 | GGGCTCCAGCCAGAGCCACCACT | 3339 | AGTGGTGGCTCTGGCTGGAGCCC |
| 3164 | TGGGCTCCAGCCAGAGCCACCAC | 3340 | GTGGTGGCTCTGGCTGGAGCCCA |
| 3165 | ATGGGCTCCAGCCAGAGCCACCA | 3341 | TGGTGGCTCTGGCTGGAGCCCAT |
| 3166 | GATGGGCTCCAGCCAGAGCCACC | 3342 | GGTGGCTCTGGCTGGAGCCCATC |
| 3167 | TGATGGGCTCCAGCCAGAGCCAC | 3343 | GTGGCTCTGGCTGGAGCCCATCA |
| 3168 | CTGATGGGCTCCAGCCAGAGCCA | 3344 | TGGCTCTGGCTGGAGCCCATCAG |
| 3169 | CCTGATGGGCTCCAGCCAGAGCC | 3345 | GGCTCTGGCTGGAGCCCATCAGG |
| 3170 | ACCTGATGGGCTCCAGCCAGAGC | 3346 | GCTCTGGCTGGAGCCCATCAGGT |
| 3171 | CACCTGATGGGCTCCAGCCAGAG | 3347 | CTCTGGCTGGAGCCCATCAGGTG |
| 3172 | ACACCTGATGGGCTCCAGCCAGA | 3348 | TCTGGCTGGAGCCCATCAGGTGT |
| 3173 | CACACCTGATGGGCTCCAGCCAG | 3349 | CTGGCTGGAGCCCATCAGGTGTG |
| 3174 | TCACACCTGATGGGCTCCAGCCA | 3350 | TGGCTGGAGCCCATCAGGTGTGA |
| 3175 | TTCACACCTGATGGGCTCCAGCC | 3351 | GGCTGGAGCCCATCAGGTGTGAA |
| 3176 | GTTCACACCTGATGGGCTCCAGC | 3352 | GCTGGAGCCCATCAGGTGTGAAC |
| 3177 | CGTTCACACCTGATGGGCTCCAG | 3353 | CTGGAGCCCATCAGGTGTGAACG |
| 3178 | TCGTTCACACCTGATGGGCTCCA | 3354 | TGGAGCCCATCAGGTGTGAACGA |
| 3179 | CTCGTTCACACCTGATGGGCTCC | 3355 | GGAGCCCATCAGGTGTGAACGAG |
| 3180 | GCTCGTTCACACCTGATGGGCTC | 3356 | GAGCCCATCAGGTGTGAACGAGC |
| 3181 | GGCTCGTTCACACCTGATGGGCT | 3357 | AGCCCATCAGGTGTGAACGAGCC |
| 3182 | AGGCTCGTTCACACCTGATGGGC | 3358 | GCCCATCAGGTGTGAACGAGCCT |
| 3183 | GAGGCTCGTTCACACCTGATGGG | 3359 | CCCATCAGGTGTGAACGAGCCTC |
| 3184 | GGAGGCTCGTTCACACCTGATGG | 3360 | CCATCAGGTGTGAACGAGCCTCC |
| 3185 | GGGAGGCTCGTTCACACCTGATG | 3361 | CATCAGGTGTGAACGAGCCTCCC |
| 3186 | AGGGAGGCTCGTTCACACCTGAT | 3362 | ATCAGGTGTGAACGAGCCTCCCT |
| 3187 | GAGGGAGGCTCGTTCACACCTGA | 3363 | TCAGGTGTGAACGAGCCTCCCTC |
| 3188 | GGAGGGAGGCTCGTTCACACCTG | 3364 | CAGGTGTGAACGAGCCTCCCTCC |
| 3189 | GGGAGGGAGGCTCGTTCACACCT | 3365 | AGGTGTGAACGAGCCTCCCTCCC |
| 3190 | GGGGAGGGAGGCTCGTTCACACC | 3366 | GGTGTGAACGAGCCTCCCTCCCC |
| 3191 | AGGGGAGGGAGGCTCGTTCACAC | 3367 | GTGTGAACGAGCCTCCCTCCCCT |
| 3192 | AAGGGGAGGGAGGCTCGTTCACA | 3368 | TGTGAACGAGCCTCCCTCCCCTT |
| 3193 | AAAGGGGAGGGAGGCTCGTTCAC | 3369 | GTGAACGAGCCTCCCTCCCCTTT |
| 3194 | GAAAGGGGAGGGAGGCTCGTTCA | 3370 | TGAACGAGCCTCCCTCCCCTTTC |
| 3195 | AGAAAGGGGAGGGAGGCTCGTTC | 3371 | GAACGAGCCTCCCTCCCCTTTCT |
| 3196 | AAGAAAGGGGAGGGAGGCTCGTT | 3372 | AACGAGCCTCCCTCCCCTTTCTT |
| 3197 | GAAGAAAGGGGAGGGAGGCTCGT | 3373 | ACGAGCCTCCCTCCCCTTTCTTC |
| 3198 | AGAAGAAAGGGGAGGGAGGCTCG | 3374 | CGAGCCTCCCTCCCCTTTCTTCT |
| 3199 | GAGAAGAAAGGGGAGGGAGGCTC | 3375 | GAGCCTCCCTCCCCTTTCTTCTC |
| 3200 | CGAGAAGAAAGGGGAGGGAGGCT | 3376 | AGCCTCCCTCCCCTTTCTTCTCG |
| 3201 | CCGAGAAGAAAGGGGAGGGAGGC | 3377 | GCCTCCCTCCCCTTTCTTCTCGG |
| 3202 | ACCGAGAAGAAAGGGGAGGGAGG | 3378 | CCTCCCTCCCCTTTCTTCTCGGT |
| 3203 | CACCGAGAAGAAAGGGGAGGGAG | 3379 | CTCCCTCCCCTTTCTTCTCGGTG |
| 3204 | GCACCGAGAAGAAAGGGGAGGGA | 3380 | TCCCTCCCCTTTCTTCTCGGTGC |
| 3205 | AGCACCGAGAAGAAAGGGGAGGG | 3381 | CCCTCCCCTTTCTTCTCGGTGCT |
| 3206 | AAGCACCGAGAAGAAAGGGGAGG | 3382 | CCTCCCCTTTCTTCTCGGTGCTT |
| 3207 | GAAGCACCGAGAAGAAAGGGGAG | 3383 | CTCCCCTTTCTTCTCGGTGCTTC |
| 3208 | AGAAGCACCGAGAAGAAAGGGGA | 3384 | TCCCCTTTCTTCTCGGTGCTTCT |
| 3209 | AAGAAGCACCGAGAAGAAAGGGG | 3385 | CCCCTTTCTTCTCGGTGCTTCTT |
| 3210 | GAAGAAGCACCGAGAAGAAAGGG | 3386 | CCCTTTCTTCTCGGTGCTTCTTC |
| 3211 | AGAAGAAGCACCGAGAAGAAAGG | 3387 | CCTTTCTTCTCGGTGCTTCTTCT |
| 3212 | GAGAAGAAGCACCGAGAAGAAAG | 3388 | CTTTCTTCTCGGTGCTTCTTCTC |
| 3213 | CGAGAAGAAGCACCGAGAAGAAA | 3389 | TTTCTTCTCGGTGCTTCTTCTCG |
| 3214 | ACGAGAAGAAGCACCGAGAAGAA | 3390 | TTCTTCTCGGTGCTTCTTCTCGT |
| 3215 | CACGAGAAGAAGCACCGAGAAGA | 3391 | TCTTCTCGGTGCTTCTTCTCGTG |
| 3216 | TCACGAGAAGAAGCACCGAGAAG | 3392 | CTTCTCGGTGCTTCTTCTCGTGA |
| 3217 | GTCACGAGAAGAAGCACCGAGAA | 3393 | TTCTCGGTGCTTCTTCTCGTGAC |
| 3218 | AGTCACGAGAAGAAGCACCGAGA | 3394 | TCTCGGTGCTTCTTCTCGTGACT |
| 3219 | AAGTCACGAGAAGAAGCACCGAG | 3395 | CTCGGTGCTTCTTCTCGTGACTT |
| 3220 | CAAGTCACGAGAAGAAGCACCGA | 3396 | TCGGTGCTTCTTCTCGTGACTTG |
| 3221 | CCAAGTCACGAGAAGAAGCACCG | 3397 | CGGTGCTTCTTCTCGTGACTTGG |

*At least one nucleoside linkage of the nucleobase sequence is selected from a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

TABLE 8B
Additional KCNQ2 Oligonucleotide Variant Sequences complementary to a
sequence a KCNQ2 transcript

| SEQ | AON Sequence* | SEQ | Target Sequence |
|---|---|---|---|
| ID NO: | (5′→3′) | ID NO: | (5′→3′) |
| 3398 | TCACCACCACCACCACCATCATC | 3900 | GATGATGGTGGTGGTGGTGGTGA |
| 3399 | ATCACCACCACCACCACCATCAT | 3901 | ATGATGGTGGTGGTGGTGGTGAT |
| 3400 | CATCACCACCACCACCACCATCA | 3902 | TGATGGTGGTGGTGGTGGTGATG |
| 3401 | CCATCACCACCACCACCACCATC | 3903 | GATGGTGGTGGTGGTGGTGATGG |
| 3402 | ACCATCACCACCACCACCACCAT | 3904 | ATGGTGGTGGTGGTGGTGATGGT |
| 3403 | CACCATCACCACCACCACCACCA | 3905 | TGGTGGTGGTGGTGGTGATGGTG |
| 3404 | TCACCATCACCACCACCACCACC | 3906 | GGTGGTGGTGGTGGTGATGGTGA |
| 3405 | ATCACCATCACCACCACCACCAC | 3907 | GTGGTGGTGGTGGTGATGGTGAT |
| 3406 | CATCACCATCACCACCACCACCA | 3908 | TGGTGGTGGTGGTGATGGTGATG |
| 3407 | CCATCACCATCACCACCACCACC | 3909 | GGTGGTGGTGGTGATGGTGATGG |
| 3408 | ACCATCACCATCACCACCACCAC | 3910 | GTGGTGGTGGTGATGGTGATGGT |
| 3409 | CACCATCACCATCACCACCACCA | 3911 | TGGTGGTGGTGATGGTGATGGTG |
| 3410 | CCACCATCACCATCACCACCACC | 3912 | GGTGGTGGTGATGGTGATGGTGG |
| 3411 | ACCACCATCACCATCACCACCAC | 3913 | GTGGTGGTGATGGTGATGGTGGT |
| 3412 | CACCACCATCACCATCACCACCA | 3914 | TGGTGGTGATGGTGATGGTGGTG |
| 3413 | TCACCACCATCACCATCACCACC | 3915 | GGTGGTGATGGTGATGGTGGTGA |
| 3414 | ATCACCACCATCACCATCACCAC | 3916 | GTGGTGATGGTGATGGTGGTGAT |
| 3415 | CATCACCACCATCACCATCACCA | 3917 | TGGTGATGGTGATGGTGGTGATG |
| 3416 | CCATCACCACCATCACCATCACC | 3918 | GGTGATGGTGATGGTGGTGATGG |
| 3417 | ACCATCACCACCATCACCATCAC | 3919 | GTGATGGTGATGGTGGTGATGGT |
| 3418 | CACCATCACCACCATCACCATCA | 3920 | TGATGGTGATGGTGGTGATGGTG |
| 3419 | TCACCATCACCACCATCACCATC | 3921 | GATGGTGATGGTGGTGATGGTGA |
| 3420 | ATCACCATCACCACCATCACCAT | 3922 | ATGGTGATGGTGGTGATGGTGAT |
| 3421 | CATCACCATCACCACCATCACCA | 3923 | TGGTGATGGTGGTGATGGTGATG |
| 3422 | CCATCACCATCACCACCATCACC | 3924 | GGTGATGGTGGTGATGGTGATGG |
| 3423 | ACCATCACCATCACCACCATCAC | 3925 | GTGATGGTGGTGATGGTGATGGT |
| 3424 | CACCATCACCATCACCACCATCA | 3926 | TGATGGTGGTGATGGTGATGGTG |
| 3425 | CCACCATCACCATCACCACCATC | 3927 | GATGGTGGTGATGGTGATGGTGG |
| 3426 | ACCACCATCACCATCACCACCAT | 3928 | ATGGTGGTGATGGTGATGGTGGT |
| 3427 | CACCACCATCACCATCACCACCA | 3929 | TGGTGGTGATGGTGATGGTGGTG |
| 3428 | CCACCACCATCACCATCACCACC | 3930 | GGTGGTGATGGTGATGGTGGTGG |
| 3429 | ACCACCACCATCACCATCACCAC | 3931 | GTGGTGATGGTGATGGTGGTGGT |
| 3430 | CACCACCACCATCACCATCACCA | 3932 | TGGTGATGGTGATGGTGGTGGTG |
| 3431 | CCACCACCACCATCACCATCACC | 3933 | GGTGATGGTGATGGTGGTGGTGG |
| 3432 | ACCACCACCACCATCACCATCAC | 3934 | GTGATGGTGATGGTGGTGGTGGT |
| 3433 | CACCACCACCACCATCACCATCA | 3935 | TGATGGTGATGGTGGTGGTGGTG |
| 3434 | CCACCACCACCACCATCACCATC | 3936 | GATGGTGATGGTGGTGGTGGTGG |
| 3435 | ACCACCACCACCACCATCACCAT | 3937 | ATGGTGATGGTGGTGGTGGTGGT |
| 3436 | CACCACCACCACCACCATCACCA | 3938 | TGGTGATGGTGGTGGTGGTGGTG |
| 3437 | CCACCACCACCACCACCATCACC | 3939 | GGTGATGGTGGTGGTGGTGGTGG |
| 3438 | ACCACCACCACCACCACCATCAC | 3940 | GTGATGGTGGTGGTGGTGGTGGT |
| 3439 | CACCACCACCACCACCACCATCA | 3941 | TGATGGTGGTGGTGGTGGTGGTG |
| 3440 | CCACCACCACCACCACCACCATC | 3942 | GATGGTGGTGGTGGTGGTGGTGG |
| 3441 | ACCACCACCACCACCACCACCAT | 3943 | ATGGTGGTGGTGGTGGTGGTGGT |
| 3442 | CACCACCACCACCACCACCACCA | 3944 | TGGTGGTGGTGGTGGTGGTGGTG |
| 3443 | TCACCACCACCACCACCACCACC | 3945 | GGTGGTGGTGGTGGTGGTGGTGA |
| 3444 | ATCACCACCACCACCACCACCAC | 3946 | GTGGTGGTGGTGGTGGTGGTGAT |
| 3445 | CATCACCACCACCACCACCACCA | 3947 | TGGTGGTGGTGGTGGTGGTGATG |
| 3446 | CCATCACCACCACCACCACCACC | 3948 | GGTGGTGGTGGTGGTGGTGATGG |
| 3447 | ACCATCACCACCACCACCACCAC | 3949 | GTGGTGGTGGTGGTGGTGATGGT |
| 3448 | CACCATCACCACCACCACCACCA | 3950 | TGGTGGTGGTGGTGGTGATGGTG |
| 3449 | CCACCATCACCACCACCACCACC | 3951 | GGTGGTGGTGGTGGTGATGGTGG |
| 3450 | ACCACCATCACCACCACCACCAC | 3952 | GTGGTGGTGGTGGTGATGGTGGT |
| 3451 | CACCACCATCACCACCACCACCA | 3953 | TGGTGGTGGTGGTGATGGTGGTG |
| 3452 | CCACCACCATCACCACCACCACC | 3954 | GGTGGTGGTGGTGATGGTGGTGG |
| 3453 | ACCACCACCATCACCACCACCAC | 3955 | GTGGTGGTGGTGATGGTGGTGGT |
| 3454 | CACCACCACCATCACCACCACCA | 3956 | TGGTGGTGGTGATGGTGGTGGTG |
| 3455 | TCACCACCACCATCACCACCACC | 3957 | GGTGGTGGTGATGGTGGTGGTGA |
| 3456 | ATCACCACCACCATCACCACCAC | 3958 | GTGGTGGTGATGGTGGTGGTGAT |
| 3457 | CATCACCACCACCATCACCACCA | 3959 | TGGTGGTGATGGTGGTGGTGATG |
| 3458 | CCATCACCACCACCATCACCACC | 3960 | GGTGGTGATGGTGGTGGTGATGG |
| 3459 | ACCATCACCACCACCATCACCAC | 3961 | GTGGTGATGGTGGTGGTGATGGT |
| 3460 | AACCATCACCACCACCATCACCA | 3962 | TGGTGATGGTGGTGGTGATGGTT |
| 3461 | AAACCATCACCACCACCATCACC | 3963 | GGTGATGGTGGTGGTGATGGTTT |
| 3462 | AAAACCATCACCACCACCATCAC | 3964 | GTGATGGTGGTGGTGATGGTTTT |
| 3463 | CAAAACCATCACCACCACCATCA | 3965 | TGATGGTGGTGGTGATGGTTTTG |
| 3464 | CCAAAACCATCACCACCACCATC | 3966 | GATGGTGGTGGTGATGGTTTTGG |
| 3465 | ACCAAAACCATCACCACCACCAT | 3967 | ATGGTGGTGGTGATGGTTTTGGT |
| 3466 | CACCAAAACCATCACCACCACCA | 3968 | TGGTGGTGGTGATGGTTTTGGTG |
| 3467 | CCACCAAAACCATCACCACCACC | 3969 | GGTGGTGGTGATGGTTTTGGTGG |
| 3468 | ACCACCAAAACCATCACCACCAC | 3970 | GTGGTGGTGATGGTTTTGGTGGT |
| 3469 | CACCACCAAAACCATCACCACCA | 3971 | TGGTGGTGATGGTTTTGGTGGTG |
| 3470 | CCACCACCAAAACCATCACCACC | 3972 | GGTGGTGATGGTTTTGGTGGTGG |
| 3471 | ACCACCACCAAAACCATCACCAC | 3973 | GTGGTGATGGTTTTGGTGGTGGT |
| 3472 | CACCACCACCAAAACCATCACCA | 3974 | TGGTGATGGTTTTGGTGGTGGTG |
| 3473 | TCACCACCACCAAAACCATCACC | 3975 | GGTGATGGTTTTGGTGGTGGTGA |
| 3474 | ATCACCACCACCAAAACCATCAC | 3976 | GTGATGGTTTTGGTGGTGGTGAT |
| 3475 | CATCACCACCACCAAAACCATCA | 3977 | TGATGGTTTTGGTGGTGGTGATG |
| 3476 | CCATCACCACCACCAAAACCATC | 3978 | GATGGTTTTGGTGGTGGTGATGG |
| 3477 | ACCATCACCACCACCAAAACCAT | 3979 | ATGGTTTTGGTGGTGGTGATGGT |
| 3478 | CACCATCACCACCACCAAAACCA | 3980 | TGGTTTTGGTGGTGGTGATGGTG |
| 3479 | CCACCATCACCACCACCAAAACC | 3981 | GGTTTTGGTGGTGGTGATGGTGG |
| 3480 | ACCACCATCACCACCACCAAAAC | 3982 | GTTTTGGTGGTGGTGATGGTGGT |
| 3481 | GACCACCATCACCACCACCAAAA | 3983 | TTTTGGTGGTGGTGATGGTGGTC |
| 3482 | TGACCACCATCACCACCACCAAA | 3984 | TTTGGTGGTGGTGATGGTGGTCA |
| 3483 | ATGACCACCATCACCACCACCAA | 3985 | TTGGTGGTGGTGATGGTGGTCAT |
| 3484 | CATGACCACCATCACCACCACCA | 3986 | TGGTGGTGGTGATGGTGGTCATG |
| 3485 | CCATGACCACCATCACCACCACC | 3987 | GGTGGTGGTGATGGTGGTCATGG |
| 3486 | ACCATGACCACCATCACCACCAC | 3988 | GTGGTGGTGATGGTGGTCATGGT |
| 3487 | CACCATGACCACCATCACCACCA | 3989 | TGGTGGTGATGGTGGTCATGGTG |
| 3488 | ACACCATGACCACCATCACCACC | 3990 | GGTGGTGATGGTGGTCATGGTGT |
| 3489 | AACACCATGACCACCATCACCAC | 3991 | GTGGTGATGGTGGTCATGGTGTT |
| 3490 | CAACACCATGACCACCATCACCA | 3992 | TGGTGATGGTGGTCATGGTGTTG |
| 3491 | TCAACACCATGACCACCATCACC | 3993 | GGTGATGGTGGTCATGGTGTTGA |
| 3492 | ATCAACACCATGACCACCATCAC | 3994 | GTGATGGTGGTCATGGTGTTGAT |
| 3493 | CATCAACACCATGACCACCATCA | 3995 | TGATGGTGGTCATGGTGTTGATG |
| 3494 | ACATCAACACCATGACCACCATC | 3996 | GATGGTGGTCATGGTGTTGATGT |
| 3495 | CACATCAACACCATGACCACCAT | 3997 | ATGGTGGTCATGGTGTTGATGTG |
| 3496 | TCACATCAACACCATGACCACCA | 3998 | TGGTGGTCATGGTGTTGATGTGA |
| 3497 | ATCACATCAACACCATGACCACC | 3999 | GGTGGTCATGGTGTTGATGTGAT |
| 3498 | CATCACATCAACACCATGACCAC | 4000 | GTGGTCATGGTGTTGATGTGATG |
| 3499 | CCATCACATCAACACCATGACCA | 4001 | TGGTCATGGTGTTGATGTGATGG |
| 3500 | ACCATCACATCAACACCATGACC | 4002 | GGTCATGGTGTTGATGTGATGGT |
| 3501 | CACCATCACATCAACACCATGAC | 4003 | GTCATGGTGTTGATGTGATGGTG |
| 3502 | CCACCATCACATCAACACCATGA | 4004 | TCATGGTGTTGATGTGATGGTGG |
| 3503 | ACCACCATCACATCAACACCATG | 4005 | CATGGTGTTGATGTGATGGTGGT |
| 3504 | CACCACCATCACATCAACACCAT | 4006 | ATGGTGTTGATGTGATGGTGGTG |
| 3505 | CCACCACCATCACATCAACACCA | 4007 | TGGTGTTGATGTGATGGTGGTGG |
| 3506 | ACCACCACCATCACATCAACACC | 4008 | GGTGTTGATGTGATGGTGGTGGT |
| 3507 | CACCACCACCATCACATCAACAC | 4009 | GTGTTGATGTGATGGTGGTGGTG |
| 3508 | ACACCACCACCATCACATCAACA | 4010 | TGTTGATGTGATGGTGGTGGTGT |
| 3509 | AACACCACCACCATCACATCAAC | 4011 | GTTGATGTGATGGTGGTGGTGTT |
| 3510 | CAACACCACCACCATCACATCAA | 4012 | TTGATGTGATGGTGGTGGTGTTG |
| 3511 | CCAACACCACCACCATCACATCA | 4013 | TGATGTGATGGTGGTGGTGTTGG |
| 3512 | ACCAACACCACCACCATCACATC | 4014 | GATGTGATGGTGGTGGTGTTGGT |
| 3513 | CACCAACACCACCACCATCACAT | 4015 | ATGTGATGGTGGTGGTGTTGGTG |
| 3514 | CCACCAACACCACCACCATCACA | 4016 | TGTGATGGTGGTGGTGTTGGTGG |
| 3515 | ACCACCAACACCACCACCATCAC | 4017 | GTGATGGTGGTGGTGTTGGTGGT |
| 3516 | CACCACCAACACCACCACCATCA | 4018 | TGATGGTGGTGGTGTTGGTGGTG |
| 3517 | CCACCACCAACACCACCACCATC | 4019 | GATGGTGGTGGTGTTGGTGGTGG |
| 3518 | ACCACCACCAACACCACCACCAT | 4020 | ATGGTGGTGGTGTTGGTGGTGGT |
| 3519 | CACCACCACCAACACCACCACCA | 4021 | TGGTGGTGGTGTTGGTGGTGGTG |
| 3520 | TCACCACCACCAACACCACCACC | 4022 | GGTGGTGGTGTTGGTGGTGGTGA |
| 3521 | ATCACCACCACCAACACCACCAC | 4023 | GTGGTGGTGTTGGTGGTGGTGAT |
| 3522 | CATCACCACCACCAACACCACCA | 4024 | TGGTGGTGTTGGTGGTGGTGATG |
| 3523 | CCATCACCACCACCAACACCACC | 4025 | GGTGGTGTTGGTGGTGGTGATGG |
| 3524 | ACCATCACCACCACCAACACCAC | 4026 | GTGGTGTTGGTGGTGGTGATGGT |
| 3525 | CACCATCACCACCACCAACACCA | 4027 | TGGTGTTGGTGGTGGTGATGGTG |
| 3526 | TCACCATCACCACCACCAACACC | 4028 | GGTGTTGGTGGTGGTGATGGTGA |
| 3527 | ATCACCATCACCACCACCAACAC | 4029 | GTGTTGGTGGTGGTGATGGTGAT |
| 3528 | CATCACCATCACCACCACCAACA | 4030 | TGTTGGTGGTGGTGATGGTGATG |
| 3529 | GCATCACCATCACCACCACCAAC | 4031 | GTTGGTGGTGGTGATGGTGATGC |
| 3530 | AGCATCACCATCACCACCACCAA | 4032 | TTGGTGGTGGTGATGGTGATGCT |
| 3531 | AAGCATCACCATCACCACCACCA | 4033 | TGGTGGTGGTGATGGTGATGCTT |
| 3532 | GAAGCATCACCATCACCACCACC | 4034 | GGTGGTGGTGATGGTGATGCTTC |
| 3533 | AGAAGCATCACCATCACCACCAC | 4035 | GTGGTGGTGATGGTGATGCTTCT |
| 3534 | CAGAAGCATCACCATCACCACCA | 4036 | TGGTGGTGATGGTGATGCTTCTG |
| 3535 | CCAGAAGCATCACCATCACCACC | 4037 | GGTGGTGATGGTGATGCTTCTGG |
| 3536 | ACCAGAAGCATCACCATCACCAC | 4038 | GTGGTGATGGTGATGCTTCTGGT |
| 3537 | CACCAGAAGCATCACCATCACCA | 4039 | TGGTGATGGTGATGCTTCTGGTG |
| 3538 | TCACCAGAAGCATCACCATCACC | 4040 | GGTGATGGTGATGCTTCTGGTGA |
| 3539 | ATCACCAGAAGCATCACCATCAC | 4041 | GTGATGGTGATGCTTCTGGTGAT |
| 3540 | CATCACCAGAAGCATCACCATCA | 4042 | TGATGGTGATGCTTCTGGTGATG |
| 3541 | CCATCACCAGAAGCATCACCATC | 4043 | GATGGTGATGCTTCTGGTGATGG |
| 3542 | ACCATCACCAGAAGCATCACCAT | 4044 | ATGGTGATGCTTCTGGTGATGGT |
| 3543 | CACCATCACCAGAAGCATCACCA | 4045 | TGGTGATGCTTCTGGTGATGGTG |
| 3544 | CCACCATCACCAGAAGCATCACC | 4046 | GGTGATGCTTCTGGTGATGGTGG |
| 3545 | ACCACCATCACCAGAAGCATCAC | 4047 | GTGATGCTTCTGGTGATGGTGGT |
| 3546 | GACCACCATCACCAGAAGCATCA | 4048 | TGATGCTTCTGGTGATGGTGGTC |
| 3547 | TGACCACCATCACCAGAAGCATC | 4049 | GATGCTTCTGGTGATGGTGGTCA |
| 3548 | ATGACCACCATCACCAGAAGCAT | 4050 | ATGCTTCTGGTGATGGTGGTCAT |
| 3549 | CATGACCACCATCACCAGAAGCA | 4051 | TGCTTCTGGTGATGGTGGTCATG |
| 3550 | TCATGACCACCATCACCAGAAGC | 4052 | GCTTCTGGTGATGGTGGTCATGA |
| 3551 | ATCATGACCACCATCACCAGAAG | 4053 | CTTCTGGTGATGGTGGTCATGAT |
| 3552 | CATCATGACCACCATCACCAGAA | 4054 | TTCTGGTGATGGTGGTCATGATG |
| 3553 | CCATCATGACCACCATCACCAGA | 4055 | TCTGGTGATGGTGGTCATGATGG |
| 3554 | ACCATCATGACCACCATCACCAG | 4056 | CTGGTGATGGTGGTCATGATGGT |
| 3555 | CACCATCATGACCACCATCACCA | 4057 | TGGTGATGGTGGTCATGATGGTG |
| 3556 | CCACCATCATGACCACCATCACC | 4058 | GGTGATGGTGGTCATGATGGTGG |
| 3557 | ACCACCATCATGACCACCATCAC | 4059 | GTGATGGTGGTCATGATGGTGGT |
| 3558 | CACCACCATCATGACCACCATCA | 4060 | TGATGGTGGTCATGATGGTGGTG |
| 3559 | GCACCACCATCATGACCACCATC | 4061 | GATGGTGGTCATGATGGTGGTGC |
| 3560 | GGCACCACCATCATGACCACCAT | 4062 | ATGGTGGTCATGATGGTGGTGCC |
| 3561 | CGGCACCACCATCATGACCACCA | 4063 | TGGTGGTCATGATGGTGGTGCCG |
| 3562 | ACGGCACCACCATCATGACCACC | 4064 | GGTGGTCATGATGGTGGTGCCGT |
| 3563 | GACGGCACCACCATCATGACCAC | 4065 | GTGGTCATGATGGTGGTGCCGTC |
| 3564 | AGACGGCACCACCATCATGACCA | 4066 | TGGTCATGATGGTGGTGCCGTCT |
| 3565 | CAGACGGCACCACCATCATGACC | 4067 | GGTCATGATGGTGGTGCCGTCTG |
| 3566 | GCAGACGGCACCACCATCATGAC | 4068 | GTCATGATGGTGGTGCCGTCTGC |
| 3567 | GGCAGACGGCACCACCATCATGA | 4069 | TCATGATGGTGGTGCCGTCTGCC |
| 3568 | AGGCAGACGGCACCACCATCATG | 4070 | CATGATGGTGGTGCCGTCTGCCT |
| 3569 | GAGGCAGACGGCACCACCATCAT | 4071 | ATGATGGTGGTGCCGTCTGCCTC |
| 3570 | AGAGGCAGACGGCACCACCATCA | 4072 | TGATGGTGGTGCCGTCTGCCTCT |
| 3571 | GAGAGGCAGACGGCACCACCATC | 4073 | GATGGTGGTGCCGTCTGCCTCTC |
| 3572 | TGAGAGGCAGACGGCACCACCAT | 4074 | ATGGTGGTGCCGTCTGCCTCTCA |
| 3573 | CTGAGAGGCAGACGGCACCACCA | 4075 | TGGTGGTGCCGTCTGCCTCTCAG |
| 3574 | CCTGAGAGGCAGACGGCACCACC | 4076 | GGTGGTGCCGTCTGCCTCTCAGG |
| 3575 | TCCTGAGAGGCAGACGGCACCAC | 4077 | GTGGTGCCGTCTGCCTCTCAGGA |
| 3576 | CTCCTGAGAGGCAGACGGCACCA | 4078 | TGGTGCCGTCTGCCTCTCAGGAG |
| 3577 | GCTCCTGAGAGGCAGACGGCACC | 4079 | GGTGCCGTCTGCCTCTCAGGAGC |
| 3578 | AGCTCCTGAGAGGCAGACGGCAC | 4080 | GTGCCGTCTGCCTCTCAGGAGCT |
| 3579 | CAGCTCCTGAGAGGCAGACGGCA | 4081 | TGCCGTCTGCCTCTCAGGAGCTG |
| 3580 | CCAGCTCCTGAGAGGCAGACGGC | 4082 | GCCGTCTGCCTCTCAGGAGCTGG |
| 3581 | ACCAGCTCCTGAGAGGCAGACGG | 4083 | CCGTCTGCCTCTCAGGAGCTGGT |
| 3582 | GACCAGCTCCTGAGAGGCAGACG | 4084 | CGTCTGCCTCTCAGGAGCTGGTC |
| 3583 | TGACCAGCTCCTGAGAGGCAGAC | 4085 | GTCTGCCTCTCAGGAGCTGGTCA |
| 3584 | GTGACCAGCTCCTGAGAGGCAGA | 4086 | TCTGCCTCTCAGGAGCTGGTCAC |
| 3585 | AGTGACCAGCTCCTGAGAGGCAG | 4087 | CTGCCTCTCAGGAGCTGGTCACT |
| 3586 | CAGTGACCAGCTCCTGAGAGGCA | 4088 | TGCCTCTCAGGAGCTGGTCACTG |
| 3587 | GCAGTGACCAGCTCCTGAGAGGC | 4089 | GCCTCTCAGGAGCTGGTCACTGC |
| 3588 | GGCAGTGACCAGCTCCTGAGAGG | 4090 | CCTCTCAGGAGCTGGTCACTGCC |
| 3589 | AGGCAGTGACCAGCTCCTGAGAG | 4091 | CTCTCAGGAGCTGGTCACTGCCT |
| 3590 | CAGGCAGTGACCAGCTCCTGAGA | 4092 | TCTCAGGAGCTGGTCACTGCCTG |
| 3591 | CCAGGCAGTGACCAGCTCCTGAG | 4093 | CTCAGGAGCTGGTCACTGCCTGG |
| 3592 | ACCAGGCAGTGACCAGCTCCTGA | 4094 | TCAGGAGCTGGTCACTGCCTGGT |
| 3593 | TACCAGGCAGTGACCAGCTCCTG | 4095 | CAGGAGCTGGTCACTGCCTGGTA |
| 3594 | GTACCAGGCAGTGACCAGCTCCT | 4096 | AGGAGCTGGTCACTGCCTGGTAC |
| 3595 | TGTACCAGGCAGTGACCAGCTCC | 4097 | GGAGCTGGTCACTGCCTGGTACA |
| 3596 | ATGTACCAGGCAGTGACCAGCTC | 4098 | GAGCTGGTCACTGCCTGGTACAT |
| 3597 | GATGTACCAGGCAGTGACCAGCT | 4099 | AGCTGGTCACTGCCTGGTACATC |
| 3598 | CGATGTACCAGGCAGTGACCAGC | 4100 | GCTGGTCACTGCCTGGTACATCG |
| 3599 | CCGATGTACCAGGCAGTGACCAG | 4101 | CTGGTCACTGCCTGGTACATCGG |
| 3600 | GCCGATGTACCAGGCAGTGACCA | 4102 | TGGTCACTGCCTGGTACATCGGC |
| 3601 | AGCCGATGTACCAGGCAGTGACC | 4103 | GGTCACTGCCTGGTACATCGGCT |
| 3602 | AAGCCGATGTACCAGGCAGTGAC | 4104 | GTCACTGCCTGGTACATCGGCTT |
| 3603 | GAAGCCGATGTACCAGGCAGTGA | 4105 | TCACTGCCTGGTACATCGGCTTC |
| 3604 | GGAAGCCGATGTACCAGGCAGTG | 4106 | CACTGCCTGGTACATCGGCTTCC |
| 3605 | AGGAAGCCGATGTACCAGGCAGT | 4107 | ACTGCCTGGTACATCGGCTTCCT |
| 3606 | AAGGAAGCCGATGTACCAGGCAG | 4108 | CTGCCTGGTACATCGGCTTCCTT |
| 3607 | AAAGGAAGCCGATGTACCAGGCA | 4109 | TGCCTGGTACATCGGCTTCCTTT |
| 3608 | CAAAGGAAGCCGATGTACCAGGC | 4110 | GCCTGGTACATCGGCTTCCTTTG |
| 3609 | ACAAAGGAAGCCGATGTACCAGG | 4111 | CCTGGTACATCGGCTTCCTTTGT |
| 3610 | GACAAAGGAAGCCGATGTACCAG | 4112 | CTGGTACATCGGCTTCCTTTGTC |
| 3611 | AGACAAAGGAAGCCGATGTACCA | 4113 | TGGTACATCGGCTTCCTTTGTCT |
| 3612 | GAGACAAAGGAAGCCGATGTACC | 4114 | GGTACATCGGCTTCCTTTGTCTC |
| 3613 | TGAGACAAAGGAAGCCGATGTAC | 4115 | GTACATCGGCTTCCTTTGTCTCA |
| 3614 | ATGAGACAAAGGAAGCCGATGTA | 4116 | TACATCGGCTTCCTTTGTCTCAT |
| 3615 | GATGAGACAAAGGAAGCCGATGT | 4117 | ACATCGGCTTCCTTTGTCTCATC |
| 3616 | GGATGAGACAAAGGAAGCCGATG | 4118 | CATCGGCTTCCTTTGTCTCATCC |
| 3617 | AGGATGAGACAAAGGAAGCCGAT | 4119 | ATCGGCTTCCTTTGTCTCATCCT |
| 3618 | CAGGATGAGACAAAGGAAGCCGA | 4120 | TCGGCTTCCTTTGTCTCATCCTG |
| 3619 | CCAGGATGAGACAAAGGAAGCCG | 4121 | CGGCTTCCTTTGTCTCATCCTGG |
| 3620 | GCCAGGATGAGACAAAGGAAGCC | 4122 | GGCTTCCTTTGTCTCATCCTGGC |
| 3621 | GGCCAGGATGAGACAAAGGAAGC | 4123 | GCTTCCTTTGTCTCATCCTGGCC |
| 3622 | AGGCCAGGATGAGACAAAGGAAG | 4124 | CTTCCTTTGTCTCATCCTGGCCT |
| 3623 | GAGGCCAGGATGAGACAAAGGAA | 4125 | TTCCTTTGTCTCATCCTGGCCTC |
| 3624 | CGAGGCCAGGATGAGACAAAGGA | 4126 | TCCTTTGTCTCATCCTGGCCTCG |
| 3625 | ACGAGGCCAGGATGAGACAAAGG | 4127 | CCTTTGTCTCATCCTGGCCTCGT |
| 3626 | AACGAGGCCAGGATGAGACAAAG | 4128 | CTTTGTCTCATCCTGGCCTCGTT |
| 3627 | GAACGAGGCCAGGATGAGACAAA | 4129 | TTTGTCTCATCCTGGCCTCGTTC |
| 3628 | GGAACGAGGCCAGGATGAGACAA | 4130 | TTGTCTCATCCTGGCCTCGTTCC |
| 3629 | AGGAACGAGGCCAGGATGAGACA | 4131 | TGTCTCATCCTGGCCTCGTTCCT |
| 3630 | CAGGAACGAGGCCAGGATGAGAC | 4132 | GTCTCATCCTGGCCTCGTTCCTG |
| 3631 | CCAGGAACGAGGCCAGGATGAGA | 4133 | TCTCATCCTGGCCTCGTTCCTGG |
| 3632 | ACCAGGAACGAGGCCAGGATGAG | 4134 | CTCATCCTGGCCTCGTTCCTGGT |
| 3633 | CACCAGGAACGAGGCCAGGATGA | 4135 | TCATCCTGGCCTCGTTCCTGGTG |
| 3634 | ACACCAGGAACGAGGCCAGGATG | 4136 | CATCCTGGCCTCGTTCCTGGTGT |
| 3635 | TACACCAGGAACGAGGCCAGGAT | 4137 | ATCCTGGCCTCGTTCCTGGTGTA |
| 3636 | GTACACCAGGAACGAGGCCAGGA | 4138 | TCCTGGCCTCGTTCCTGGTGTAC |
| 3637 | AGTACACCAGGAACGAGGCCAGG | 4139 | CCTGGCCTCGTTCCTGGTGTACT |
| 3638 | AAGTACACCAGGAACGAGGCCAG | 4140 | CTGGCCTCGTTCCTGGTGTACTT |
| 3639 | CAAGTACACCAGGAACGAGGCCA | 4141 | TGGCCTCGTTCCTGGTGTACTTG |
| 3640 | CCAAGTACACCAGGAACGAGGCC | 4142 | GGCCTCGTTCCTGGTGTACTTGG |
| 3641 | GCCAAGTACACCAGGAACGAGGC | 4143 | GCCTCGTTCCTGGTGTACTTGGC |
| 3642 | TGCCAAGTACACCAGGAACGAGG | 4144 | CCTCGTTCCTGGTGTACTTGGCA |
| 3643 | CTGCCAAGTACACCAGGAACGAG | 4145 | CTCGTTCCTGGTGTACTTGGCAG |
| 3644 | TCTGCCAAGTACACCAGGAACGA | 4146 | TCGTTCCTGGTGTACTTGGCAGA |
| 3645 | CTCTGCCAAGTACACCAGGAACG | 4147 | CGTTCCTGGTGTACTTGGCAGAG |
| 3646 | TCTCTGCCAAGTACACCAGGAAC | 4148 | GTTCCTGGTGTACTTGGCAGAGA |
| 3647 | TTCTCTGCCAAGTACACCAGGAA | 4149 | TTCCTGGTGTACTTGGCAGAGAA |
| 3648 | CTTCTCTGCCAAGTACACCAGGA | 4150 | TCCTGGTGTACTTGGCAGAGAAG |
| 3649 | CCTTCTCTGCCAAGTACACCAGG | 4151 | CCTGGTGTACTTGGCAGAGAAGG |
| 3650 | CCCTTCTCTGCCAAGTACACCAG | 4152 | CTGGTGTACTTGGCAGAGAAGGG |
| 3651 | CCCCTTCTCTGCCAAGTACACCA | 4153 | TGGTGTACTTGGCAGAGAAGGGG |
| 3652 | CCCCCTTCTCTGCCAAGTACACC | 4154 | GGTGTACTTGGCAGAGAAGGGGG |
| 3653 | TCCCCCTTCTCTGCCAAGTACAC | 4155 | GTGTACTTGGCAGAGAAGGGGGA |
| 3654 | CTCCCCCTTCTCTGCCAAGTACA | 4156 | TGTACTTGGCAGAGAAGGGGGAG |
| 3655 | TCTCCCCCTTCTCTGCCAAGTAC | 4157 | GTACTTGGCAGAGAAGGGGGAGA |
| 3656 | TTCTCCCCCTTCTCTGCCAAGTA | 4158 | TACTTGGCAGAGAAGGGGGAGAA |
| 3657 | GTTCTCCCCCTTCTCTGCCAAGT | 4159 | ACTTGGCAGAGAAGGGGGAGAAC |
| 3658 | CGTTCTCCCCCTTCTCTGCCAAG | 4160 | CTTGGCAGAGAAGGGGGAGAACG |
| 3659 | TCGTTCTCCCCCTTCTCTGCCAA | 4161 | TTGGCAGAGAAGGGGGAGAACGA |
| 3660 | GTCGTTCTCCCCCTTCTCTGCCA | 4162 | TGGCAGAGAAGGGGGAGAACGAC |
| 3661 | GGTCGTTCTCCCCCTTCTCTGCC | 4163 | GGCAGAGAAGGGGGAGAACGACC |
| 3662 | TGGTCGTTCTCCCCCTTCTCTGC | 4164 | GCAGAGAAGGGGGAGAACGACCA |
| 3663 | GTGGTCGTTCTCCCCCTTCTCTG | 4165 | CAGAGAAGGGGGAGAACGACCAC |
| 3664 | AGTGGTCGTTCTCCCCCTTCTCT | 4166 | AGAGAAGGGGGAGAACGACCACT |
| 3665 | AAGTGGTCGTTCTCCCCCTTCTC | 4167 | GAGAAGGGGGAGAACGACCACTT |
| 3666 | AAAGTGGTCGTTCTCCCCCTTCT | 4168 | AGAAGGGGGAGAACGACCACTTT |
| 3667 | CAAAGTGGTCGTTCTCCCCCTTC | 4169 | GAAGGGGGAGAACGACCACTTTG |
| 3668 | TCAAAGTGGTCGTTCTCCCCCTT | 4170 | AAGGGGGAGAACGACCACTTTGA |
| 3669 | GTCAAAGTGGTCGTTCTCCCCCT | 4171 | AGGGGGAGAACGACCACTTTGAC |
| 3670 | TGTCAAAGTGGTCGTTCTCCCCC | 4172 | GGGGGAGAACGACCACTTTGACA |
| 3671 | GTGTCAAAGTGGTCGTTCTCCCC | 4173 | GGGGAGAACGACCACTTTGACAC |
| 3672 | GGTGTCAAAGTGGTCGTTCTCCC | 4174 | GGGAGAACGACCACTTTGACACC |
| 3673 | AGGTGTCAAAGTGGTCGTTCTCC | 4175 | GGAGAACGACCACTTTGACACCT |
| 3674 | TAGGTGTCAAAGTGGTCGTTCTC | 4176 | GAGAACGACCACTTTGACACCTA |
| 3675 | GTAGGTGTCAAAGTGGTCGTTCT | 4177 | AGAACGACCACTTTGACACCTAC |
| 3676 | CGTAGGTGTCAAAGTGGTCGTTC | 4178 | GAACGACCACTTTGACACCTACG |
| 3677 | GCGTAGGTGTCAAAGTGGTCGTT | 4179 | AACGACCACTTTGACACCTACGC |
| 3678 | CGCGTAGGTGTCAAAGTGGTCGT | 4180 | ACGACCACTTTGACACCTACGCG |
| 3679 | CCGCGTAGGTGTCAAAGTGGTCG | 4181 | CGACCACTTTGACACCTACGCGG |
| 3680 | TCCGCGTAGGTGTCAAAGTGGTC | 4182 | GACCACTTTGACACCTACGCGGA |
| 3681 | ATCCGCGTAGGTGTCAAAGTGGT | 4183 | ACCACTTTGACACCTACGCGGAT |
| 3682 | CATCCGCGTAGGTGTCAAAGTGG | 4184 | CCACTTTGACACCTACGCGGATG |
| 3683 | GCATCCGCGTAGGTGTCAAAGTG | 4185 | CACTTTGACACCTACGCGGATGC |
| 3684 | TGCATCCGCGTAGGTGTCAAAGT | 4186 | ACTTTGACACCTACGCGGATGCA |
| 3685 | GTGCATCCGCGTAGGTGTCAAAG | 4187 | CTTTGACACCTACGCGGATGCAC |
| 3686 | AGTGCATCCGCGTAGGTGTCAAA | 4188 | TTTGACACCTACGCGGATGCACT |
| 3687 | GAGTGCATCCGCGTAGGTGTCAA | 4189 | TTGACACCTACGCGGATGCACTC |
| 3688 | AGAGTGCATCCGCGTAGGTGTCA | 4190 | TGACACCTACGCGGATGCACTCT |
| 3689 | CAGAGTGCATCCGCGTAGGTGTC | 4191 | GACACCTACGCGGATGCACTCTG |
| 3690 | CCAGAGTGCATCCGCGTAGGTGT | 4192 | ACACCTACGCGGATGCACTCTGG |
| 3691 | ACCAGAGTGCATCCGCGTAGGTG | 4193 | CACCTACGCGGATGCACTCTGGT |
| 3692 | CACCAGAGTGCATCCGCGTAGGT | 4194 | ACCTACGCGGATGCACTCTGGTG |
| 3693 | CCACCAGAGTGCATCCGCGTAGG | 4195 | CCTACGCGGATGCACTCTGGTGG |
| 3694 | CCCACCAGAGTGCATCCGCGTAG | 4196 | CTACGCGGATGCACTCTGGTGGG |
| 3695 | CCCCACCAGAGTGCATCCGCGTA | 4197 | TACGCGGATGCACTCTGGTGGGG |
| 3696 | GCCCCACCAGAGTGCATCCGCGT | 4198 | ACGCGGATGCACTCTGGTGGGGC |
| 3697 | GGCCCCACCAGAGTGCATCCGCG | 4199 | CGCGGATGCACTCTGGTGGGGCC |
| 3698 | AGGCCCCACCAGAGTGCATCCGC | 4200 | GCGGATGCACTCTGGTGGGGCCT |
| 3699 | CAGGCCCCACCAGAGTGCATCCG | 4201 | CGGATGCACTCTGGTGGGGCCTG |
| 3700 | CCAGGCCCCACCAGAGTGCATCC | 4202 | GGATGCACTCTGGTGGGGCCTGG |
| 3701 | ACCAGGCCCCACCAGAGTGCATC | 4203 | GATGCACTCTGGTGGGGCCTGGT |
| 3702 | CACCAGGCCCCACCAGAGTGCAT | 4204 | ATGCACTCTGGTGGGGCCTGGTG |
| 3703 | TCACCAGGCCCCACCAGAGTGCA | 4205 | TGCACTCTGGTGGGGCCTGGTGA |
| 3704 | CTCACCAGGCCCCACCAGAGTGC | 4206 | GCACTCTGGTGGGGCCTGGTGAG |
| 3705 | ACTCACCAGGCCCCACCAGAGTG | 4207 | CACTCTGGTGGGGCCTGGTGAGT |
| 3706 | AACTCACCAGGCCCCACCAGAGT | 4208 | ACTCTGGTGGGGCCTGGTGAGTT |
| 3707 | CAACTCACCAGGCCCCACCAGAG | 4209 | CTCTGGTGGGGCCTGGTGAGTTG |
| 3708 | ACAACTCACCAGGCCCCACCAGA | 4210 | TCTGGTGGGGCCTGGTGAGTTGT |
| 3709 | CACAACTCACCAGGCCCCACCAG | 4211 | CTGGTGGGGCCTGGTGAGTTGTG |
| 3710 | CCACAACTCACCAGGCCCCACCA | 4212 | TGGTGGGGCCTGGTGAGTTGTGG |
| 3711 | ACCACAACTCACCAGGCCCCACC | 4213 | GGTGGGGCCTGGTGAGTTGTGGT |
| 3712 | GACCACAACTCACCAGGCCCCAC | 4214 | GTGGGGCCTGGTGAGTTGTGGTC |
| 3713 | TGACCACAACTCACCAGGCCCCA | 4215 | TGGGGCCTGGTGAGTTGTGGTCA |
| 3714 | ATGACCACAACTCACCAGGCCCC | 4216 | GGGGCCTGGTGAGTTGTGGTCAT |
| 3715 | AATGACCACAACTCACCAGGCCC | 4217 | GGGCCTGGTGAGTTGTGGTCATT |
| 3716 | CAATGACCACAACTCACCAGGCC | 4218 | GGCCTGGTGAGTTGTGGTCATTG |
| 3717 | ACAATGACCACAACTCACCAGGC | 4219 | GCCTGGTGAGTTGTGGTCATTGT |
| 3718 | CACAATGACCACAACTCACCAGG | 4220 | CCTGGTGAGTTGTGGTCATTGTG |
| 3719 | CCACAATGACCACAACTCACCAG | 4221 | CTGGTGAGTTGTGGTCATTGTGG |
| 3720 | ACCACAATGACCACAACTCACCA | 4222 | TGGTGAGTTGTGGTCATTGTGGT |
| 3721 | AACCACAATGACCACAACTCACC | 4223 | GGTGAGTTGTGGTCATTGTGGTT |
| 3722 | AAACCACAATGACCACAACTCAC | 4224 | GTGAGTTGTGGTCATTGTGGTTT |
| 3723 | AAAACCACAATGACCACAACTCA | 4225 | TGAGTTGTGGTCATTGTGGTTTT |
| 3724 | GAAAACCACAATGACCACAACTC | 4226 | GAGTTGTGGTCATTGTGGTTTTC |
| 3725 | GGAAAACCACAATGACCACAACT | 4227 | AGTTGTGGTCATTGTGGTTTTCC |
| 3726 | GGGAAAACCACAATGACCACAAC | 4228 | GTTGTGGTCATTGTGGTTTTCCC |
| 3727 | AGGGAAAACCACAATGACCACAA | 4229 | TTGTGGTCATTGTGGTTTTCCCT |
| 3728 | AAGGGAAAACCACAATGACCACA | 4230 | TGTGGTCATTGTGGTTTTCCCTT |
| 3729 | AAAGGGAAAACCACAATGACCAC | 4231 | GTGGTCATTGTGGTTTTCCCTTT |
| 3730 | GAAAGGGAAAACCACAATGACCA | 4232 | TGGTCATTGTGGTTTTCCCTTTC |
| 3731 | GGAAAGGGAAAACCACAATGACC | 4233 | GGTCATTGTGGTTTTCCCTTTCC |
| 3732 | GGGAAAGGGAAAACCACAATGAC | 4234 | GTCATTGTGGTTTTCCCTTTCCC |
| 3733 | AGGGAAAGGGAAAACCACAATGA | 4235 | TCATTGTGGTTTTCCCTTTCCCT |
| 3734 | CAGGGAAAGGGAAAACCACAATG | 4236 | CATTGTGGTTTTCCCTTTCCCTG |
| 3735 | GCAGGGAAAGGGAAAACCACAAT | 4237 | ATTGTGGTTTTCCCTTTCCCTGC |
| 3736 | AGCAGGGAAAGGGAAAACCACAA | 4238 | TTGTGGTTTTCCCTTTCCCTGCT |
| 3737 | CAGCAGGGAAAGGGAAAACCACA | 4239 | TGTGGTTTTCCCTTTCCCTGCTG |
| 3738 | TCAGCAGGGAAAGGGAAAACCAC | 4240 | GTGGTTTTCCCTTTCCCTGCTGA |
| 3739 | ATCAGCAGGGAAAGGGAAAACCA | 4241 | TGGTTTTCCCTTTCCCTGCTGAT |
| 3740 | TATCAGCAGGGAAAGGGAAAACC | 4242 | GGTTTTCCCTTTCCCTGCTGATA |
| 3741 | GTATCAGCAGGGAAAGGGAAAAC | 4243 | GTTTTCCCTTTCCCTGCTGATAC |
| 3742 | TGTATCAGCAGGGAAAGGGAAAA | 4244 | TTTTCCCTTTCCCTGCTGATACA |
| 3743 | GTGTATCAGCAGGGAAAGGGAAA | 4245 | TTTCCCTTTCCCTGCTGATACAC |
| 3744 | GGTGTATCAGCAGGGAAAGGGAA | 4246 | TTCCCTTTCCCTGCTGATACACC |
| 3745 | GGGTGTATCAGCAGGGAAAGGGA | 4247 | TCCCTTTCCCTGCTGATACACCC |
| 3746 | GGGGTGTATCAGCAGGGAAAGGG | 4248 | CCCTTTCCCTGCTGATACACCCC |
| 3747 | AGGGGTGTATCAGCAGGGAAAGG | 4249 | CCTTTCCCTGCTGATACACCCCT |
| 3748 | CAGGGGTGTATCAGCAGGGAAAG | 4250 | CTTTCCCTGCTGATACACCCCTG |
| 3749 | ACAGGGGTGTATCAGCAGGGAAA | 4251 | TTTCCCTGCTGATACACCCCTGT |
| 3750 | GACAGGGGTGTATCAGCAGGGAA | 4252 | TTCCCTGCTGATACACCCCTGTC |
| 3751 | GGACAGGGGTGTATCAGCAGGGA | 4253 | TCCCTGCTGATACACCCCTGTCC |
| 3752 | GGGACAGGGGTGTATCAGCAGGG | 4254 | CCCTGCTGATACACCCCTGTCCC |
| 3753 | AGGGACAGGGGTGTATCAGCAGG | 4255 | CCTGCTGATACACCCCTGTCCCT |
| 3754 | CAGGGACAGGGGTGTATCAGCAG | 4256 | CTGCTGATACACCCCTGTCCCTG |
| 3755 | ACAGGGACAGGGGTGTATCAGCA | 4257 | TGCTGATACACCCCTGTCCCTGT |
| 3756 | CACAGGGACAGGGGTGTATCAGC | 4258 | GCTGATACACCCCTGTCCCTGTG |
| 3757 | GCACAGGGACAGGGGTGTATCAG | 4259 | CTGATACACCCCTGTCCCTGTGC |
| 3758 | AGCACAGGGACAGGGGTGTATCA | 4260 | TGATACACCCCTGTCCCTGTGCT |
| 3759 | CAGCACAGGGACAGGGGTGTATC | 4261 | GATACACCCCTGTCCCTGTGCTG |
| 3760 | CCAGCACAGGGACAGGGGTGTAT | 4262 | ATACACCCCTGTCCCTGTGCTGG |
| 3761 | CCCAGCACAGGGACAGGGGTGTA | 4263 | TACACCCCTGTCCCTGTGCTGGG |
| 3762 | TCCCAGCACAGGGACAGGGGTGT | 4264 | ACACCCCTGTCCCTGTGCTGGGA |
| 3763 | GTCCCAGCACAGGGACAGGGGTG | 4265 | CACCCCTGTCCCTGTGCTGGGAC |
| 3764 | GGTCCCAGCACAGGGACAGGGGT | 4266 | ACCCCTGTCCCTGTGCTGGGACC |
| 3765 | TGGTCCCAGCACAGGGACAGGGG | 4267 | CCCCTGTCCCTGTGCTGGGACCA |
| 3766 | CTGGTCCCAGCACAGGGACAGGG | 4268 | CCCTGTCCCTGTGCTGGGACCAG |
| 3767 | CCTGGTCCCAGCACAGGGACAGG | 4269 | CCTGTCCCTGTGCTGGGACCAGG |
| 3768 | GCCTGGTCCCAGCACAGGGACAG | 4270 | CTGTCCCTGTGCTGGGACCAGGC |
| 3769 | AGCCTGGTCCCAGCACAGGGACA | 4271 | TGTCCCTGTGCTGGGACCAGGCT |
| 3770 | GAGCCTGGTCCCAGCACAGGGAC | 4272 | GTCCCTGTGCTGGGACCAGGCTC |
| 3771 | AGAGCCTGGTCCCAGCACAGGGA | 4273 | TCCCTGTGCTGGGACCAGGCTCT |
| 3772 | GAGAGCCTGGTCCCAGCACAGGG | 4274 | CCCTGTGCTGGGACCAGGCTCTC |
| 3773 | TGAGAGCCTGGTCCCAGCACAGG | 4275 | CCTGTGCTGGGACCAGGCTCTCA |
| 3774 | GTGAGAGCCTGGTCCCAGCACAG | 4276 | CTGTGCTGGGACCAGGCTCTCAC |
| 3775 | AGTGAGAGCCTGGTCCCAGCACA | 4277 | TGTGCTGGGACCAGGCTCTCACT |
| 3776 | CAGTGAGAGCCTGGTCCCAGCAC | 4278 | GTGCTGGGACCAGGCTCTCACTG |
| 3777 | CCAGTGAGAGCCTGGTCCCAGCA | 4279 | TGCTGGGACCAGGCTCTCACTGG |
| 3778 | GCCAGTGAGAGCCTGGTCCCAGC | 4280 | GCTGGGACCAGGCTCTCACTGGC |
| 3779 | AGCCAGTGAGAGCCTGGTCCCAG | 4281 | CTGGGACCAGGCTCTCACTGGCT |
| 3780 | CAGCCAGTGAGAGCCTGGTCCCA | 4282 | TGGGACCAGGCTCTCACTGGCTG |
| 3781 | TCAGCCAGTGAGAGCCTGGTCCC | 4283 | GGGACCAGGCTCTCACTGGCTGA |
| 3782 | CTCAGCCAGTGAGAGCCTGGTCC | 4284 | GGACCAGGCTCTCACTGGCTGAG |
| 3783 | GCTCAGCCAGTGAGAGCCTGGTC | 4285 | GACCAGGCTCTCACTGGCTGAGC |
| 3784 | GGCTCAGCCAGTGAGAGCCTGGT | 4286 | ACCAGGCTCTCACTGGCTGAGCC |
| 3785 | AGGCTCAGCCAGTGAGAGCCTGG | 4287 | CCAGGCTCTCACTGGCTGAGCCT |
| 3786 | CAGGCTCAGCCAGTGAGAGCCTG | 4288 | CAGGCTCTCACTGGCTGAGCCTG |
| 3787 | GCAGGCTCAGCCAGTGAGAGCCT | 4289 | AGGCTCTCACTGGCTGAGCCTGC |
| 3788 | AGCAGGCTCAGCCAGTGAGAGCC | 4290 | GGCTCTCACTGGCTGAGCCTGCT |
| 3789 | GAGCAGGCTCAGCCAGTGAGAGC | 4291 | GCTCTCACTGGCTGAGCCTGCTC |
| 3790 | GGAGCAGGCTCAGCCAGTGAGAG | 4292 | CTCTCACTGGCTGAGCCTGCTCC |
| 3791 | TGGAGCAGGCTCAGCCAGTGAGA | 4293 | TCTCACTGGCTGAGCCTGCTCCA |
| 3792 | ATGGAGCAGGCTCAGCCAGTGAG | 4294 | CTCACTGGCTGAGCCTGCTCCAT |
| 3793 | TATGGAGCAGGCTCAGCCAGTGA | 4295 | TCACTGGCTGAGCCTGCTCCATA |
| 3794 | GTATGGAGCAGGCTCAGCCAGTG | 4296 | CACTGGCTGAGCCTGCTCCATAC |
| 3795 | TGTATGGAGCAGGCTCAGCCAGT | 4297 | ACTGGCTGAGCCTGCTCCATACA |
| 3796 | ATGTATGGAGCAGGCTCAGCCAG | 4298 | CTGGCTGAGCCTGCTCCATACAT |
| 3797 | GATGTATGGAGCAGGCTCAGCCA | 4299 | TGGCTGAGCCTGCTCCATACATC |
| 3798 | AGATGTATGGAGCAGGCTCAGCC | 4300 | GGCTGAGCCTGCTCCATACATCT |
| 3799 | GAGATGTATGGAGCAGGCTCAGC | 4301 | GCTGAGCCTGCTCCATACATCTC |
| 3800 | AGAGATGTATGGAGCAGGCTCAG | 4302 | CTGAGCCTGCTCCATACATCTCT |
| 3801 | AAGAGATGTATGGAGCAGGCTCA | 4303 | TGAGCCTGCTCCATACATCTCTT |
| 3802 | AAAGAGATGTATGGAGCAGGCTC | 4304 | GAGCCTGCTCCATACATCTCTTT |
| 3803 | CAAAGAGATGTATGGAGCAGGCT | 4305 | AGCCTGCTCCATACATCTCTTTG |
| 3804 | CCAAAGAGATGTATGGAGCAGGC | 4306 | GCCTGCTCCATACATCTCTTTGG |
| 3805 | CCCAAAGAGATGTATGGAGCAGG | 4307 | CCTGCTCCATACATCTCTTTGGG |
| 3806 | CCCCAAAGAGATGTATGGAGCAG | 4308 | CTGCTCCATACATCTCTTTGGGG |
| 3807 | GCCCCAAAGAGATGTATGGAGCA | 4309 | TGCTCCATACATCTCTTTGGGGC |
| 3808 | GGCCCCAAAGAGATGTATGGAGC | 4310 | GCTCCATACATCTCTTTGGGGCC |
| 3809 | TGGCCCCAAAGAGATGTATGGAG | 4311 | CTCCATACATCTCTTTGGGGCCA |
| 3810 | GTGGCCCCAAAGAGATGTATGGA | 4312 | TCCATACATCTCTTTGGGGCCAC |
| 3811 | GGTGGCCCCAAAGAGATGTATGG | 4313 | CCATACATCTCTTTGGGGCCACC |
| 3812 | AGGTGGCCCCAAAGAGATGTATG | 4314 | CATACATCTCTTTGGGGCCACCT |
| 3813 | CAGGTGGCCCCAAAGAGATGTAT | 4315 | ATACATCTCTTTGGGGCCACCTG |
| 3814 | GCAGGTGGCCCCAAAGAGATGTA | 4316 | TACATCTCTTTGGGGCCACCTGC |
| 3815 | AGCAGGTGGCCCCAAAGAGATGT | 4317 | ACATCTCTTTGGGGCCACCTGCT |
| 3816 | CAGCAGGTGGCCCCAAAGAGATG | 4318 | CATCTCTTTGGGGCCACCTGCTG |
| 3817 | CCAGCAGGTGGCCCCAAAGAGAT | 4319 | ATCTCTTTGGGGCCACCTGCTGG |
| 3818 | GCCAGCAGGTGGCCCCAAAGAGA | 4320 | TCTCTTTGGGGCCACCTGCTGGC |
| 3819 | AGCCAGCAGGTGGCCCCAAAGAG | 4321 | CTCTTTGGGGCCACCTGCTGGCT |
| 3820 | CAGCCAGCAGGTGGCCCCAAAGA | 4322 | TCTTTGGGGCCACCTGCTGGCTG |
| 3821 | GCAGCCAGCAGGTGGCCCCAAAG | 4323 | CTTTGGGGCCACCTGCTGGCTGC |
| 3822 | GGCAGCCAGCAGGTGGCCCCAAA | 4324 | TTTGGGGCCACCTGCTGGCTGCC |
| 3823 | GGGCAGCCAGCAGGTGGCCCCAA | 4325 | TTGGGGCCACCTGCTGGCTGCCC |
| 3824 | CGGGCAGCCAGCAGGTGGCCCCA | 4326 | TGGGGCCACCTGCTGGCTGCCCG |
| 3825 | ACGGGCAGCCAGCAGGTGGCCCC | 4327 | GGGGCCACCTGCTGGCTGCCCGT |
| 3826 | CACGGGCAGCCAGCAGGTGGCCC | 4328 | GGGCCACCTGCTGGCTGCCCGTG |
| 3827 | CCACGGGCAGCCAGCAGGTGGCC | 4329 | GGCCACCTGCTGGCTGCCCGTGG |
| 3828 | ACCACGGGCAGCCAGCAGGTGGC | 4330 | GCCACCTGCTGGCTGCCCGTGGT |
| 3829 | GACCACGGGCAGCCAGCAGGTGG | 4331 | CCACCTGCTGGCTGCCCGTGGTC |
| 3830 | TGACCACGGGCAGCCAGCAGGTG | 4332 | CACCTGCTGGCTGCCCGTGGTCA |
| 3831 | ATGACCACGGGCAGCCAGCAGGT | 4333 | ACCTGCTGGCTGCCCGTGGTCAT |
| 3832 | CATGACCACGGGCAGCCAGCAGG | 4334 | CCTGCTGGCTGCCCGTGGTCATG |
| 3833 | TCATGACCACGGGCAGCCAGCAG | 4335 | CTGCTGGCTGCCCGTGGTCATGA |
| 3834 | ATCATGACCACGGGCAGCCAGCA | 4336 | TGCTGGCTGCCCGTGGTCATGAT |
| 3835 | CATCATGACCACGGGCAGCCAGC | 4337 | GCTGGCTGCCCGTGGTCATGATG |
| 3836 | CCATCATGACCACGGGCAGCCAG | 4338 | CTGGCTGCCCGTGGTCATGATGG |
| 3837 | GCCATCATGACCACGGGCAGCCA | 4339 | TGGCTGCCCGTGGTCATGATGGC |
| 3838 | AGCCATCATGACCACGGGCAGCC | 4340 | GGCTGCCCGTGGTCATGATGGCT |
| 3839 | AAGCCATCATGACCACGGGCAGC | 4341 | GCTGCCCGTGGTCATGATGGCTT |
| 3840 | CAAGCCATCATGACCACGGGCAG | 4342 | CTGCCCGTGGTCATGATGGCTTG |
| 3841 | ACAAGCCATCATGACCACGGGCA | 4343 | TGCCCGTGGTCATGATGGCTTGT |
| 3842 | CACAAGCCATCATGACCACGGGC | 4344 | GCCCGTGGTCATGATGGCTTGTG |
| 3843 | CCACAAGCCATCATGACCACGGG | 4345 | CCCGTGGTCATGATGGCTTGTGG |
| 3844 | ACCACAAGCCATCATGACCACGG | 4346 | CCGTGGTCATGATGGCTTGTGGT |
| 3845 | GACCACAAGCCATCATGACCACG | 4347 | CGTGGTCATGATGGCTTGTGGTC |
| 3846 | CGACCACAAGCCATCATGACCAC | 4348 | GTGGTCATGATGGCTTGTGGTCG |
| 3847 | TCGACCACAAGCCATCATGACCA | 4349 | TGGTCATGATGGCTTGTGGTCGA |
| 3848 | CTCGACCACAAGCCATCATGACC | 4350 | GGTCATGATGGCTTGTGGTCGAG |
| 3849 | CCTCGACCACAAGCCATCATGAC | 4351 | GTCATGATGGCTTGTGGTCGAGG |
| 3850 | GCCTCGACCACAAGCCATCATGA | 4352 | TCATGATGGCTTGTGGTCGAGGC |
| 3851 | CGCCTCGACCACAAGCCATCATG | 4353 | CATGATGGCTTGTGGTCGAGGCG |
| 3852 | CCGCCTCGACCACAAGCCATCAT | 4354 | ATGATGGCTTGTGGTCGAGGCGG |
| 3853 | CCCGCCTCGACCACAAGCCATCA | 4355 | TGATGGCTTGTGGTCGAGGCGGG |
| 3854 | CCCCGCCTCGACCACAAGCCATC | 4356 | GATGGCTTGTGGTCGAGGCGGGG |
| 3855 | ACCCCGCCTCGACCACAAGCCAT | 4357 | ATGGCTTGTGGTCGAGGCGGGGT |
| 3856 | CACCCCGCCTCGACCACAAGCCA | 4358 | TGGCTTGTGGTCGAGGCGGGGTG |
| 3857 | CCACCCCGCCTCGACCACAAGCC | 4359 | GGCTTGTGGTCGAGGCGGGGTGG |
| 3858 | ACCACCCCGCCTCGACCACAAGC | 4360 | GCTTGTGGTCGAGGCGGGGTGGT |
| 3859 | CACCACCCCGCCTCGACCACAAG | 4361 | CTTGTGGTCGAGGCGGGGTGGTG |
| 3860 | CCACCACCCCGCCTCGACCACAA | 4362 | TTGTGGTCGAGGCGGGGTGGTGG |
| 3861 | ACCACCACCCCGCCTCGACCACA | 4363 | TGTGGTCGAGGCGGGGTGGTGGT |
| 3862 | AACCACCACCCCGCCTCGACCAC | 4364 | GTGGTCGAGGCGGGGTGGTGGTT |
| 3863 | CAACCACCACCCCGCCTCGACCA | 4365 | TGGTCGAGGCGGGGTGGTGGTTG |
| 3864 | GCAACCACCACCCCGCCTCGACC | 4366 | GGTCGAGGCGGGGTGGTGGTTGC |
| 3865 | GGCAACCACCACCCCGCCTCGAC | 4367 | GTCGAGGCGGGGTGGTGGTTGCC |
| 3866 | TGGCAACCACCACCCCGCCTCGA | 4368 | TCGAGGCGGGGTGGTGGTTGCCA |
| 3867 | TTGGCAACCACCACCCCGCCTCG | 4369 | CGAGGCGGGGTGGTGGTTGCCAA |
| 3868 | CTTGGCAACCACCACCCCGCCTC | 4370 | GAGGCGGGGTGGTGGTTGCCAAG |
| 3869 | GCTTGGCAACCACCACCCCGCCT | 4371 | AGGCGGGGTGGTGGTTGCCAAGC |
| 3870 | AGCTTGGCAACCACCACCCCGCC | 4372 | GGCGGGGTGGTGGTTGCCAAGCT |
| 3871 | CAGCTTGGCAACCACCACCCCGC | 4373 | GCGGGGTGGTGGTTGCCAAGCTG |
| 3872 | CCAGCTTGGCAACCACCACCCCG | 4374 | CGGGGTGGTGGTTGCCAAGCTGG |
| 3873 | GCCAGCTTGGCAACCACCACCCC | 4375 | GGGGTGGTGGTTGCCAAGCTGGC |
| 3874 | CGCCAGCTTGGCAACCACCACCC | 4376 | GGGTGGTGGTTGCCAAGCTGGCG |
| 3875 | CCGCCAGCTTGGCAACCACCACC | 4377 | GGTGGTGGTTGCCAAGCTGGCGG |
| 3876 | CCCGCCAGCTTGGCAACCACCAC | 4378 | GTGGTGGTTGCCAAGCTGGCGGG |
| 3877 | CCCCGCCAGCTTGGCAACCACCA | 4379 | TGGTGGTTGCCAAGCTGGCGGGG |
| 3878 | CCCCCGCCAGCTTGGCAACCACC | 4380 | GGTGGTTGCCAAGCTGGCGGGGG |
| 3879 | TCCCCCGCCAGCTTGGCAACCAC | 4381 | GTGGTTGCCAAGCTGGCGGGGGA |
| 3880 | CTCCCCCGCCAGCTTGGCAACCA | 4382 | TGGTTGCCAAGCTGGCGGGGGAG |
| 3881 | ACTCCCCCGCCAGCTTGGCAACC | 4383 | GGTTGCCAAGCTGGCGGGGGAGT |
| 3882 | CACTCCCCCGCCAGCTTGGCAAC | 4384 | GTTGCCAAGCTGGCGGGGGAGTG |
| 3883 | CCACTCCCCCGCCAGCTTGGCAA | 4385 | TTGCCAAGCTGGCGGGGGAGTGG |
| 3884 | CCCACTCCCCCGCCAGCTTGGCA | 4386 | TGCCAAGCTGGCGGGGGAGTGGG |
| 3885 | ACCCACTCCCCCGCCAGCTTGGC | 4387 | GCCAAGCTGGCGGGGGAGTGGGT |
| 3886 | CACCCACTCCCCCGCCAGCTTGG | 4388 | CCAAGCTGGCGGGGGAGTGGGTG |
| 3887 | CCACCCACTCCCCCGCCAGCTTG | 4389 | CAAGCTGGCGGGGGAGTGGGTGG |
| 3888 | CCCACCCACTCCCCCGCCAGCTT | 4390 | AAGCTGGCGGGGGAGTGGGTGGG |
| 3889 | CCCCACCCACTCCCCCGCCAGCT | 4391 | AGCTGGCGGGGGAGTGGGTGGGG |
| 3890 | TCCCCACCCACTCCCCCGCCAGC | 4392 | GCTGGCGGGGGAGTGGGTGGGGA |
| 3891 | CTCCCCACCCACTCCCCCGCCAG | 4393 | CTGGCGGGGGAGTGGGTGGGGAG |
| 3892 | CCTCCCCACCCACTCCCCCGCCA | 4394 | TGGCGGGGGAGTGGGTGGGGAGG |
| 3893 | CCCTCCCCACCCACTCCCCCGCC | 4395 | GGCGGGGGAGTGGGTGGGGAGGG |
| 3894 | GCCCTCCCCACCCACTCCCCCGC | 4396 | GCGGGGGAGTGGGTGGGGAGGGC |
| 3895 | AGCCCTCCCCACCCACTCCCCCG | 4397 | CGGGGGAGTGGGTGGGGAGGGCT |
| 3896 | CAGCCCTCCCCACCCACTCCCCC | 4398 | GGGGGAGTGGGTGGGGAGGGCTG |
| 3897 | CCAGCCCTCCCCACCCACTCCCC | 4399 | GGGGAGTGGGTGGGGAGGGCTGG |
| 3898 | CCCAGCCCTCCCCACCCACTCCC | 4400 | GGGAGTGGGTGGGGAGGGCTGGG |
| 3899 | GCCCAGCCCTCCCCACCCACTCC | 4401 | GGAGTGGGTGGGGAGGGCTGGGC |

*At least one nucleoside linkage of the nucleobase sequence is selected from a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

### UNC13A Oligonucleotides Complementary to UNC13A Transcript

In some embodiments, a UNC13A AON includes a sequence that is at least 85% complementary to a sequence that shares at least 90% (e.g., 90%, 91%, 92%, 93%, 94%, 95%, 96%, 97%, 98%, 99%, or 100%) identity to a region of a UNC13A transcript (e.g., SEQ ID NO: 9587-9595). In some embodiments, a UNC13A AON includes a sequence that is between 90-95% complementary to a sequence that shares at least 90% (e.g., 90%, 91%, 92%, 93%, 94%, 95%, 96%, 97%, 98%, 99%, or 100%) identity to a region of a UNC13A transcript (e.g., SEQ ID NO: 9587-9595). In particular embodiments, a UNC13A AON includes a sequence that is between 85% and 90% complementary to a sequence that shares at least 90% (e.g., 90%, 91%, 92%, 93%, 94%, 95%, 96%, 97%, 98%, 99%, or 100%) identity to a region of a UNC13A transcript (e.g., SEQ ID NO: 9587-9595). In particular embodiments, a UNC13A AON includes a sequence that is between 84% to 88% complementary to a sequence that shares at least 90% (e.g., 90%, 91%, 92%, 93%, 94%, 95%, 96%, 97%, 98%, 99%, or 100%) identity to a region of a UNC13A transcript (e.g., SEQ ID NO: 9587-9595). In particular embodiments, a UNC13A AON includes a sequence that is between 89% to 92% complementary to a sequence that shares at least 90% (e.g., 90%, 91%, 92%, 93%, 94%, 95%, 96%, 97%, 98%, 99%, or 100%) identity to a region of a mis-spliced UNC13A transcript (e.g., SEQ ID NO: 9587-9595). In particular embodiments, a UNC13A AON includes a sequence that is between 94% to 96% complementary to a sequence that shares at least 90% (e.g., 90%, 91%, 92%, 93%, 94%, 95%, 96%, 97%, 98%, 99%, or 100%) identity to a region of a UNC13A transcript (e.g., SEQ ID NO: 9587-9595).

In various embodiments, a UNC13A AON comprises a sequence that shares at least 85% identity with an equal length portion of any one of SEQ ID NOs: 4531-5794, SEQ ID NOs: 7059-8322, SEQ ID NOs: 9596-9696, and SEQ ID NOs: 10670-10779. In various embodiments, a UNC13A AON comprises a sequence that shares at least 90% identity with an equal length portion of any one of SEQ ID NOs: 4531-5794, SEQ ID NOs: 7059-8322, SEQ ID NOs: 9596-9696, and SEQ ID NOs: 10670-10779.

In some embodiments, the UNC13A AON comprises a spacer and has a segment having at most 7 linked nucleosides. In some embodiments, the UNC13A AON comprises a spacer and has a segment having at most 6, 5, 4, 3, or 2 linked nucleosides.

UNC13A AON binding specificity can be assessed via measurement of parameters such as dissociation constant, melting temperature, or other criteria such as changes in protein or RNA expression levels or other assays that measure UNC13A activity or expression.

In some embodiments, a UNC13A AON can include a non-duplexed oligonucleotide. In some embodiments, a UNC13A AON can include a duplex of two oligonucleotides where the first oligonucleotide includes a nucleobase sequence that is completely or almost completely complementary to a UNC13A pre-mRNA sequence and the second oligonucleotide includes a nucleobase sequence that is complementary to the nucleobase sequence of the first oligonucleotide.

In some embodiments, a UNC13A AON can target UNC13A pre-mRNAs produced from UNC13A genes of one or more species. For example, a UNC13A AON can target a UNC13A pre-mRNA of a mammalian UNC13A gene, for example, a human (i.e., Homo sapiens) UNC13A gene. In particular embodiments, the UNC13A AON targets a human UNC13A pre-mRNA. In some embodiments, the UNC13A AON includes a nucleobase sequence that is complementary to a nucleobase sequence of a UNC13A gene or a UNC13A pre-mRNA or a portion thereof.

UNC13A AONs described herein include antisense oligonucleotides comprising the oligonucleotide sequences listed in Table 9 below:

TABLE 9
Example UNC13A AON Sequences.

| SEQ ID | AON Sequence* | SEQ ID | Target Sequence |
|---|---|---|---|
| NO: | (5′→3′) | NO: | (5′→3′) |
| 4531 | GGAGGGGCCGAGCAATGACCCTCAC | 5795 | GTGAGGGTCATTGCTCGGCCCCTCC |
| 4532 | GGGAGGGGCCGAGCAATGACCCTCA | 5796 | TGAGGGTCATTGCTCGGCCCCTCCC |
| 4533 | TGGGAGGGGCCGAGCAATGACCCTC | 5797 | GAGGGTCATTGCTCGGCCCCTCCCA |
| 4534 | ATGGGAGGGGCCGAGCAATGACCCT | 5798 | AGGGTCATTGCTCGGCCCCTCCCAT |
| 4535 | CATGGGAGGGGCCGAGCAATGACCC | 5799 | GGGTCATTGCTCGGCCCCTCCCATG |
| 4536 | GCATGGGAGGGGCCGAGCAATGACC | 5800 | GGTCATTGCTCGGCCCCTCCCATGC |
| 4537 | GGCATGGGAGGGGCCGAGCAATGAC | 5801 | GTCATTGCTCGGCCCCTCCCATGCC |
| 4538 | TGGCATGGGAGGGGCCGAGCAATGA | 5802 | TCATTGCTCGGCCCCTCCCATGCCA |
| 4539 | GTGGCATGGGAGGGGCCGAGCAATG | 5803 | CATTGCTCGGCCCCTCCCATGCCAC |
| 4540 | AGTGGCATGGGAGGGGCCGAGCAAT | 5804 | ATTGCTCGGCCCCTCCCATGCCACT |
| 4541 | AAGTGGCATGGGAGGGGCCGAGCAA | 5805 | TTGCTCGGCCCCTCCCATGCCACTT |
| 4542 | GAAGTGGCATGGGAGGGGCCGAGCA | 5806 | TGCTCGGCCCCTCCCATGCCACTTC |
| 4543 | GGAAGTGGCATGGGAGGGGCCGAGC | 5807 | GCTCGGCCCCTCCCATGCCACTTCC |
| 4544 | TGGAAGTGGCATGGGAGGGGCCGAG | 5808 | CTCGGCCCCTCCCATGCCACTTCCA |
| 4545 | GTGGAAGTGGCATGGGAGGGGCCGA | 5809 | TCGGCCCCTCCCATGCCACTTCCAC |
| 4546 | AGTGGAAGTGGCATGGGAGGGGCCG | 5810 | CGGCCCCTCCCATGCCACTTCCACT |
| 4547 | GAGTGGAAGTGGCATGGGAGGGGCC | 5811 | GGCCCCTCCCATGCCACTTCCACTC |
| 4548 | TGAGTGGAAGTGGCATGGGAGGGGC | 5812 | GCCCCTCCCATGCCACTTCCACTCA |
| 4549 | GTGAGTGGAAGTGGCATGGGAGGGG | 5813 | CCCCTCCCATGCCACTTCCACTCAC |
| 4550 | GGTGAGTGGAAGTGGCATGGGAGGG | 5814 | CCCTCCCATGCCACTTCCACTCACC |
| 4551 | TGGTGAGTGGAAGTGGCATGGGAGG | 5815 | CCTCCCATGCCACTTCCACTCACCA |
| 4552 | ATGGTGAGTGGAAGTGGCATGGGAG | 5816 | CTCCCATGCCACTTCCACTCACCAT |
| 4553 | AATGGTGAGTGGAAGTGGCATGGGA | 5817 | TCCCATGCCACTTCCACTCACCATT |
| 4554 | GAATGGTGAGTGGAAGTGGCATGGG | 5818 | CCCATGCCACTTCCACTCACCATTC |
| 4555 | GGAATGGTGAGTGGAAGTGGCATGG | 5819 | CCATGCCACTTCCACTCACCATTCC |
| 4556 | AGGAATGGTGAGTGGAAGTGGCATG | 5820 | CATGCCACTTCCACTCACCATTCCT |
| 4557 | CAGGAATGGTGAGTGGAAGTGGCAT | 5821 | ATGCCACTTCCACTCACCATTCCTG |
| 4558 | GCAGGAATGGTGAGTGGAAGTGGCA | 5822 | TGCCACTTCCACTCACCATTCCTGC |
| 4559 | GGCAGGAATGGTGAGTGGAAGTGGC | 5823 | GCCACTTCCACTCACCATTCCTGCC |
| 4560 | AGGCAGGAATGGTGAGTGGAAGTGG | 5824 | CCACTTCCACTCACCATTCCTGCCT |
| 4561 | CAGGCAGGAATGGTGAGTGGAAGTG | 5825 | CACTTCCACTCACCATTCCTGCCTG |
| 4562 | GCAGGCAGGAATGGTGAGTGGAAGT | 5826 | ACTTCCACTCACCATTCCTGCCTGC |
| 4563 | GGCAGGCAGGAATGGTGAGTGGAAG | 5827 | CTTCCACTCACCATTCCTGCCTGCC |
| 4564 | GGGCAGGCAGGAATGGTGAGTGGAA | 5828 | TTCCACTCACCATTCCTGCCTGCCC |
| 4565 | TGGGCAGGCAGGAATGGTGAGTGGA | 5829 | TCCACTCACCATTCCTGCCTGCCCA |
| 4566 | CTGGGCAGGCAGGAATGGTGAGTGG | 5830 | CCACTCACCATTCCTGCCTGCCCAG |
| 4567 | GCTGGGCAGGCAGGAATGGTGAGTG | 5831 | CACTCACCATTCCTGCCTGCCCAGC |
| 4568 | AGCTGGGCAGGCAGGAATGGTGAGT | 5832 | ACTCACCATTCCTGCCTGCCCAGCT |
| 4569 | GAGCTGGGCAGGCAGGAATGGTGAG | 5833 | CTCACCATTCCTGCCTGCCCAGCTC |
| 4570 | AGAGCTGGGCAGGCAGGAATGGTGA | 5834 | TCACCATTCCTGCCTGCCCAGCTCT |
| 4571 | AAGAGCTGGGCAGGCAGGAATGGTG | 5835 | CACCATTCCTGCCTGCCCAGCTCTT |
| 4572 | GAAGAGCTGGGCAGGCAGGAATGGT | 5836 | ACCATTCCTGCCTGCCCAGCTCTTC |
| 4573 | GGAAGAGCTGGGCAGGCAGGAATGG | 5837 | CCATTCCTGCCTGCCCAGCTCTTCC |
| 4574 | AGGAAGAGCTGGGCAGGCAGGAATG | 5838 | CATTCCTGCCTGCCCAGCTCTTCCT |
| 4575 | GAGGAAGAGCTGGGCAGGCAGGAAT | 5839 | ATTCCTGCCTGCCCAGCTCTTCCTC |
| 4576 | AGAGGAAGAGCTGGGCAGGCAGGAA | 5840 | TTCCTGCCTGCCCAGCTCTTCCTCT |
| 4577 | AAGAGGAAGAGCTGGGCAGGCAGGA | 5841 | TCCTGCCTGCCCAGCTCTTCCTCTT |
| 4578 | AAAGAGGAAGAGCTGGGCAGGCAGG | 5842 | CCTGCCTGCCCAGCTCTTCCTCTTT |
| 4579 | GAAAGAGGAAGAGCTGGGCAGGCAG | 5843 | CTGCCTGCCCAGCTCTTCCTCTTTC |
| 4580 | AGAAAGAGGAAGAGCTGGGCAGGCA | 5844 | TGCCTGCCCAGCTCTTCCTCTTTCT |
| 4581 | CAGAAAGAGGAAGAGCTGGGCAGGC | 5845 | GCCTGCCCAGCTCTTCCTCTTTCTG |
| 4582 | CCAGAAAGAGGAAGAGCTGGGCAGG | 5846 | CCTGCCCAGCTCTTCCTCTTTCTGG |
| 4583 | GCCAGAAAGAGGAAGAGCTGGGCAG | 5847 | CTGCCCAGCTCTTCCTCTTTCTGGC |
| 4584 | GGCCAGAAAGAGGAAGAGCTGGGCA | 5848 | TGCCCAGCTCTTCCTCTTTCTGGCC |
| 4585 | TGGCCAGAAAGAGGAAGAGCTGGGC | 5849 | GCCCAGCTCTTCCTCTTTCTGGCCA |
| 4586 | GTGGCCAGAAAGAGGAAGAGCTGGG | 5850 | CCCAGCTCTTCCTCTTTCTGGCCAC |
| 4587 | TGTGGCCAGAAAGAGGAAGAGCTGG | 5851 | CCAGCTCTTCCTCTTTCTGGCCACA |
| 4588 | GTGTGGCCAGAAAGAGGAAGAGCTG | 5852 | CAGCTCTTCCTCTTTCTGGCCACAC |
| 4589 | GGTGTGGCCAGAAAGAGGAAGAGCT | 5853 | AGCTCTTCCTCTTTCTGGCCACACC |
| 4590 | TGGTGTGGCCAGAAAGAGGAAGAGC | 5854 | GCTCTTCCTCTTTCTGGCCACACCA |
| 4591 | ATGGTGTGGCCAGAAAGAGGAAGAG | 5855 | CTCTTCCTCTTTCTGGCCACACCAT |
| 4592 | GATGGTGTGGCCAGAAAGAGGAAGA | 5856 | TCTTCCTCTTTCTGGCCACACCATC |
| 4593 | GGATGGTGTGGCCAGAAAGAGGAAG | 5857 | CTTCCTCTTTCTGGCCACACCATCC |
| 4594 | TGGATGGTGTGGCCAGAAAGAGGAA | 5858 | TTCCTCTTTCTGGCCACACCATCCA |
| 4595 | GTGGATGGTGTGGCCAGAAAGAGGA | 5859 | TCCTCTTTCTGGCCACACCATCCAC |
| 4596 | TGTGGATGGTGTGGCCAGAAAGAGG | 5860 | CCTCTTTCTGGCCACACCATCCACA |
| 4597 | GTGTGGATGGTGTGGCCAGAAAGAG | 5861 | CTCTTTCTGGCCACACCATCCACAC |
| 4598 | AGTGTGGATGGTGTGGCCAGAAAGA | 5862 | TCTTTCTGGCCACACCATCCACACT |
| 4599 | GAGTGTGGATGGTGTGGCCAGAAAG | 5863 | CTTTCTGGCCACACCATCCACACTC |
| 4600 | AGAGTGTGGATGGTGTGGCCAGAAA | 5864 | TTTCTGGCCACACCATCCACACTCT |
| 4601 | GAGAGTGTGGATGGTGTGGCCAGAA | 5865 | TTCTGGCCACACCATCCACACTCTC |
| 4602 | GGAGAGTGTGGATGGTGTGGCCAGA | 5866 | TCTGGCCACACCATCCACACTCTCC |
| 4603 | AGGAGAGTGTGGATGGTGTGGCCAG | 5867 | CTGGCCACACCATCCACACTCTCCT |
| 4604 | CAGGAGAGTGTGGATGGTGTGGCCA | 5868 | TGGCCACACCATCCACACTCTCCTG |
| 4605 | CCAGGAGAGTGTGGATGGTGTGGCC | 5869 | GGCCACACCATCCACACTCTCCTGG |
| 4606 | GCCAGGAGAGTGTGGATGGTGTGGC | 5870 | GCCACACCATCCACACTCTCCTGGC |
| 4607 | GGCCAGGAGAGTGTGGATGGTGTGG | 5871 | CCACACCATCCACACTCTCCTGGCC |
| 4608 | GGGCCAGGAGAGTGTGGATGGTGTG | 5872 | CACACCATCCACACTCTCCTGGCCC |
| 4609 | AGGGCCAGGAGAGTGTGGATGGTGT | 5873 | ACACCATCCACACTCTCCTGGCCCT |
| 4610 | GAGGGCCAGGAGAGTGTGGATGGTG | 5874 | CACCATCCACACTCTCCTGGCCCTC |
| 4611 | AGAGGGCCAGGAGAGTGTGGATGGT | 5875 | ACCATCCACACTCTCCTGGCCCTCT |
| 4612 | CAGAGGGCCAGGAGAGTGTGGATGG | 5876 | CCATCCACACTCTCCTGGCCCTCTG |
| 4613 | TCAGAGGGCCAGGAGAGTGTGGATG | 5877 | CATCCACACTCTCCTGGCCCTCTGA |
| 4614 | CTCAGAGGGCCAGGAGAGTGTGGAT | 5878 | ATCCACACTCTCCTGGCCCTCTGAG |
| 4615 | TCTCAGAGGGCCAGGAGAGTGTGGA | 5879 | TCCACACTCTCCTGGCCCTCTGAGA |
| 4616 | GTCTCAGAGGGCCAGGAGAGTGTGG | 5880 | CCACACTCTCCTGGCCCTCTGAGAC |
| 4617 | AGTCTCAGAGGGCCAGGAGAGTGTG | 5881 | CACACTCTCCTGGCCCTCTGAGACT |
| 4618 | CAGTCTCAGAGGGCCAGGAGAGTGT | 5882 | ACACTCTCCTGGCCCTCTGAGACTG |
| 4619 | GCAGTCTCAGAGGGCCAGGAGAGTG | 5883 | CACTCTCCTGGCCCTCTGAGACTGC |
| 4620 | GGCAGTCTCAGAGGGCCAGGAGAGT | 5884 | ACTCTCCTGGCCCTCTGAGACTGCC |
| 4621 | GGGCAGTCTCAGAGGGCCAGGAGAG | 5885 | CTCTCCTGGCCCTCTGAGACTGCCC |
| 4622 | CGGGCAGTCTCAGAGGGCCAGGAGA | 5886 | TCTCCTGGCCCTCTGAGACTGCCCG |
| 4623 | GCGGGCAGTCTCAGAGGGCCAGGAG | 5887 | CTCCTGGCCCTCTGAGACTGCCCGC |
| 4624 | GGCGGGCAGTCTCAGAGGGCCAGGA | 5888 | TCCTGGCCCTCTGAGACTGCCCGCC |
| 4625 | TGGCGGGCAGTCTCAGAGGGCCAGG | 5889 | CCTGGCCCTCTGAGACTGCCCGCCA |
| 4626 | ATGGCGGGCAGTCTCAGAGGGCCAG | 5890 | CTGGCCCTCTGAGACTGCCCGCCAT |
| 4627 | CATGGCGGGCAGTCTCAGAGGGCCA | 5891 | TGGCCCTCTGAGACTGCCCGCCATG |
| 4628 | GCATGGCGGGCAGTCTCAGAGGGCC | 5892 | GGCCCTCTGAGACTGCCCGCCATGC |
| 4629 | GGCATGGCGGGCAGTCTCAGAGGGC | 5893 | GCCCTCTGAGACTGCCCGCCATGCC |
| 4630 | TGGCATGGCGGGCAGTCTCAGAGGG | 5894 | CCCTCTGAGACTGCCCGCCATGCCA |
| 4631 | ATGGCATGGCGGGCAGTCTCAGAGG | 5895 | CCTCTGAGACTGCCCGCCATGCCAT |
| 4632 | AATGGCATGGCGGGCAGTCTCAGAG | 5896 | CTCTGAGACTGCCCGCCATGCCATT |
| 4633 | GAATGGCATGGCGGGCAGTCTCAGA | 5897 | TCTGAGACTGCCCGCCATGCCATTC |
| 4634 | GGAATGGCATGGGGGCAGTCTCAG | 5898 | CTGAGACTGCCCGCCATGCCATTCC |
| 4635 | GGGAATGGCATGGCGGGCAGTCTCA | 5899 | TGAGACTGCCCGCCATGCCATTCCC |
| 4636 | AGGGAATGGCATGGCGGGCAGTCTC | 5900 | GAGACTGCCCGCCATGCCATTCCCT |
| 4637 | AAGGGAATGGCATGGCGGGCAGTCT | 5901 | AGACTGCCCGCCATGCCATTCCCTT |
| 4638 | AAAGGGAATGGCATGGCGGGCAGTC | 5902 | GACTGCCCGCCATGCCATTCCCTTT |
| 4639 | TAAAGGGAATGGCATGGCGGGCAGT | 5903 | ACTGCCCGCCATGCCATTCCCTTTA |
| 4640 | GTAAAGGGAATGGCATGGCGGGCAG | 5904 | CTGCCCGCCATGCCATTCCCTTTAC |
| 4641 | GGTAAAGGGAATGGCATGGCGGGCA | 5905 | TGCCCGCCATGCCATTCCCTTTACC |
| 4642 | AGGTAAAGGGAATGGCATGGCGGGC | 5906 | GCCCGCCATGCCATTCCCTTTACCT |
| 4643 | CAGGTAAAGGGAATGGCATGGCGGG | 5907 | CCCGCCATGCCATTCCCTTTACCTG |
| 4644 | CCAGGTAAAGGGAATGGCATGGCGG | 5908 | CCGCCATGCCATTCCCTTTACCTGG |
| 4645 | TCCAGGTAAAGGGAATGGCATGGCG | 5909 | CGCCATGCCATTCCCTTTACCTGGA |
| 4646 | TTCCAGGTAAAGGGAATGGCATGGC | 5910 | GCCATGCCATTCCCTTTACCTGGAA |
| 4647 | TTTCCAGGTAAAGGGAATGGCATGG | 5911 | CCATGCCATTCCCTTTACCTGGAAA |
| 4648 | TTTTCCAGGTAAAGGGAATGGCATG | 5912 | CATGCCATTCCCTTTACCTGGAAAA |
| 4649 | GTTTTCCAGGTAAAGGGAATGGCAT | 5913 | ATGCCATTCCCTTTACCTGGAAAAC |
| 4650 | AGTTTTCCAGGTAAAGGGAATGGCA | 5914 | TGCCATTCCCTTTACCTGGAAAACT |
| 4651 | GAGTTTTCCAGGTAAAGGGAATGGC | 5915 | GCCATTCCCTTTACCTGGAAAACTC |
| 4652 | GGAGTTTTCCAGGTAAAGGGAATGG | 5916 | CCATTCCCTTTACCTGGAAAACTCC |
| 4653 | AGGAGTTTTCCAGGTAAAGGGAATG | 5917 | CATTCCCTTTACCTGGAAAACTCCT |
| 4654 | GAGGAGTTTTCCAGGTAAAGGGAAT | 5918 | ATTCCCTTTACCTGGAAAACTCCTC |
| 4655 | GGAGGAGTTTTCCAGGTAAAGGGAA | 5919 | TTCCCTTTACCTGGAAAACTCCTCC |
| 4656 | GGGAGGAGTTTTCCAGGTAAAGGGA | 5920 | TCCCTTTACCTGGAAAACTCCTCCC |
| 4657 | AGGGAGGAGTTTTCCAGGTAAAGGG | 5921 | CCCTTTACCTGGAAAACTCCTCCCT |
| 4658 | TAGGGAGGAGTTTTCCAGGTAAAGG | 5922 | CCTTTACCTGGAAAACTCCTCCCTA |
| 4659 | ATAGGGAGGAGTTTTCCAGGTAAAG | 5923 | CTTTACCTGGAAAACTCCTCCCTAT |
| 4660 | GATAGGGAGGAGTTTTCCAGGTAAA | 5924 | TTTACCTGGAAAACTCCTCCCTATC |
| 4661 | GGATAGGGAGGAGTTTTCCAGGTAA | 5925 | TTACCTGGAAAACTCCTCCCTATCC |
| 4662 | TGGATAGGGAGGAGTTTTCCAGGTA | 5926 | TACCTGGAAAACTCCTCCCTATCCA |
| 4663 | ATGGATAGGGAGGAGTTTTCCAGGT | 5927 | ACCTGGAAAACTCCTCCCTATCCAT |
| 4664 | GATGGATAGGGAGGAGTTTTCCAGG | 5928 | CCTGGAAAACTCCTCCCTATCCATC |
| 4665 | TGATGGATAGGGAGGAGTTTTCCAG | 5929 | CTGGAAAACTCCTCCCTATCCATCA |
| 4666 | TTGATGGATAGGGAGGAGTTTTCCA | 5930 | TGGAAAACTCCTCCCTATCCATCAA |
| 4667 | TTTGATGGATAGGGAGGAGTTTTCC | 5931 | GGAAAACTCCTCCCTATCCATCAAA |
| 4668 | CTTTGATGGATAGGGAGGAGTTTTC | 5932 | GAAAACTCCTCCCTATCCATCAAAG |
| 4669 | ACTTTGATGGATAGGGAGGAGTTTT | 5933 | AAAACTCCTCCCTATCCATCAAAGT |
| 4670 | GACTTTGATGGATAGGGAGGAGTTT | 5934 | AAACTCCTCCCTATCCATCAAAGTC |
| 4671 | GGACTTTGATGGATAGGGAGGAGTT | 5935 | AACTCCTCCCTATCCATCAAAGTCC |
| 4672 | TGGACTTTGATGGATAGGGAGGAGT | 5936 | ACTCCTCCCTATCCATCAAAGTCCA |
| 4673 | CTGGACTTTGATGGATAGGGAGGAG | 5937 | CTCCTCCCTATCCATCAAAGTCCAG |
| 4674 | TCTGGACTTTGATGGATAGGGAGGA | 5938 | TCCTCCCTATCCATCAAAGTCCAGA |
| 4675 | ATCTGGACTTTGATGGATAGGGAGG | 5939 | CCTCCCTATCCATCAAAGTCCAGAT |
| 4676 | AATCTGGACTTTGATGGATAGGGAG | 5940 | CTCCCTATCCATCAAAGTCCAGATT |
| 4677 | GAATCTGGACTTTGATGGATAGGGA | 5941 | TCCCTATCCATCAAAGTCCAGATTC |
| 4678 | TGAATCTGGACTTTGATGGATAGGG | 5942 | CCCTATCCATCAAAGTCCAGATTCA |
| 4679 | CTGAATCTGGACTTTGATGGATAGG | 5943 | CCTATCCATCAAAGTCCAGATTCAG |
| 4680 | CCTGAATCTGGACTTTGATGGATAG | 5944 | CTATCCATCAAAGTCCAGATTCAGG |
| 4681 | CCCTGAATCTGGACTTTGATGGATA | 5945 | TATCCATCAAAGTCCAGATTCAGGG |
| 4682 | ACCCTGAATCTGGACTTTGATGGAT | 5946 | ATCCATCAAAGTCCAGATTCAGGGT |
| 4683 | GACCCTGAATCTGGACTTTGATGGA | 5947 | TCCATCAAAGTCCAGATTCAGGGTC |
| 4684 | TGACCCTGAATCTGGACTTTGATGG | 5948 | CCATCAAAGTCCAGATTCAGGGTCA |
| 4685 | GTGACCCTGAATCTGGACTTTGATG | 5949 | CATCAAAGTCCAGATTCAGGGTCAC |
| 4686 | GGTGACCCTGAATCTGGACTTTGAT | 5950 | ATCAAAGTCCAGATTCAGGGTCACC |
| 4687 | AGGTGACCCTGAATCTGGACTTTGA | 5951 | TCAAAGTCCAGATTCAGGGTCACCT |
| 4688 | GAGGTGACCCTGAATCTGGACTTTG | 5952 | CAAAGTCCAGATTCAGGGTCACCTC |
| 4689 | GGAGGTGACCCTGAATCTGGACTTT | 5953 | AAAGTCCAGATTCAGGGTCACCTCC |
| 4690 | AGGAGGTGACCCTGAATCTGGACTT | 5954 | AAGTCCAGATTCAGGGTCACCTCCT |
| 4691 | GAGGAGGTGACCCTGAATCTGGACT | 5955 | AGTCCAGATTCAGGGTCACCTCCTC |
| 4692 | AGAGGAGGTGACCCTGAATCTGGAC | 5956 | GTCCAGATTCAGGGTCACCTCCTCT |
| 4693 | CAGAGGAGGTGACCCTGAATCTGGA | 5957 | TCCAGATTCAGGGTCACCTCCTCTG |
| 4694 | CCAGAGGAGGTGACCCTGAATCTGG | 5958 | CCAGATTCAGGGTCACCTCCTCTGG |
| 4695 | CCCAGAGGAGGTGACCCTGAATCTG | 5959 | CAGATTCAGGGTCACCTCCTCTGGG |
| 4696 | TCCCAGAGGAGGTGACCCTGAATCT | 5960 | AGATTCAGGGTCACCTCCTCTGGGA |
| 4697 | TTCCCAGAGGAGGTGACCCTGAATC | 5961 | GATTCAGGGTCACCTCCTCTGGGAA |
| 4698 | CTTCCCAGAGGAGGTGACCCTGAAT | 5962 | ATTCAGGGTCACCTCCTCTGGGAAG |
| 4699 | GCTTCCCAGAGGAGGTGACCCTGAA | 5963 | TTCAGGGTCACCTCCTCTGGGAAGC |
| 4700 | GGCTTCCCAGAGGAGGTGACCCTGA | 5964 | TCAGGGTCACCTCCTCTGGGAAGCC |
| 4701 | GGGCTTCCCAGAGGAGGTGACCCTG | 5965 | CAGGGTCACCTCCTCTGGGAAGCCC |
| 4702 | TGGGCTTCCCAGAGGAGGTGACCCT | 5966 | AGGGTCACCTCCTCTGGGAAGCCCA |
| 4703 | GTGGGCTTCCCAGAGGAGGTGACCC | 5967 | GGGTCACCTCCTCTGGGAAGCCCAC |
| 4704 | GGTGGGCTTCCCAGAGGAGGTGACC | 5968 | GGTCACCTCCTCTGGGAAGCCCACC |
| 4705 | AGGTGGGCTTCCCAGAGGAGGTGAC | 5969 | GTCACCTCCTCTGGGAAGCCCACCT |
| 4706 | AAGGTGGGCTTCCCAGAGGAGGTGA | 5970 | TCACCTCCTCTGGGAAGCCCACCTT |
| 4707 | CAAGGTGGGCTTCCCAGAGGAGGTG | 5971 | CACCTCCTCTGGGAAGCCCACCTTG |
| 4708 | CCAAGGTGGGCTTCCCAGAGGAGGT | 5972 | ACCTCCTCTGGGAAGCCCACCTTGG |
| 4709 | GCCAAGGTGGGCTTCCCAGAGGAGG | 5973 | CCTCCTCTGGGAAGCCCACCTTGGC |
| 4710 | GGCCAAGGTGGGCTTCCCAGAGGAG | 5974 | CTCCTCTGGGAAGCCCACCTTGGCC |
| 4711 | AGGCCAAGGTGGGCTTCCCAGAGGA | 5975 | TCCTCTGGGAAGCCCACCTTGGCCT |
| 4712 | GAGGCCAAGGTGGGCTTCCCAGAGG | 5976 | CCTCTGGGAAGCCCACCTTGGCCTC |
| 4713 | GGAGGCCAAGGTGGGCTTCCCAGAG | 5977 | CTCTGGGAAGCCCACCTTGGCCTCC |
| 4714 | TGGAGGCCAAGGTGGGCTTCCCAGA | 5978 | TCTGGGAAGCCCACCTTGGCCTCCA |
| 4715 | CTGGAGGCCAAGGTGGGCTTCCCAG | 5979 | CTGGGAAGCCCACCTTGGCCTCCAG |
| 4716 | CCTGGAGGCCAAGGTGGGCTTCCCA | 5980 | TGGGAAGCCCACCTTGGCCTCCAGG |
| 4717 | ACCTGGAGGCCAAGGTGGGCTTCCC | 5981 | GGGAAGCCCACCTTGGCCTCCAGGT |
| 4718 | AACCTGGAGGCCAAGGTGGGCTTCC | 5982 | GGAAGCCCACCTTGGCCTCCAGGTT |
| 4719 | CAACCTGGAGGCCAAGGTGGGCTTC | 5983 | GAAGCCCACCTTGGCCTCCAGGTTG |
| 4720 | TCAACCTGGAGGCCAAGGTGGGCTT | 5984 | AAGCCCACCTTGGCCTCCAGGTTGA |
| 4721 | GTCAACCTGGAGGCCAAGGTGGGCT | 5985 | AGCCCACCTTGGCCTCCAGGTTGAC |
| 4722 | AGTCAACCTGGAGGCCAAGGTGGGC | 5986 | GCCCACCTTGGCCTCCAGGTTGACT |
| 4723 | GAGTCAACCTGGAGGCCAAGGTGGG | 5987 | CCCACCTTGGCCTCCAGGTTGACTC |
| 4724 | AGAGTCAACCTGGAGGCCAAGGTGG | 5988 | CCACCTTGGCCTCCAGGTTGACTCT |
| 4725 | GAGAGTCAACCTGGAGGCCAAGGTG | 5989 | CACCTTGGCCTCCAGGTTGACTCTC |
| 4726 | TGAGAGTCAACCTGGAGGCCAAGGT | 5990 | ACCTTGGCCTCCAGGTTGACTCTCA |
| 4727 | GTGAGAGTCAACCTGGAGGCCAAGG | 5991 | CCTTGGCCTCCAGGTTGACTCTCAC |
| 4728 | AGTGAGAGTCAACCTGGAGGCCAAG | 5992 | CTTGGCCTCCAGGTTGACTCTCACT |
| 4729 | TAGTGAGAGTCAACCTGGAGGCCAA | 5993 | TTGGCCTCCAGGTTGACTCTCACTA |
| 4730 | GTAGTGAGAGTCAACCTGGAGGCCA | 5994 | TGGCCTCCAGGTTGACTCTCACTAC |
| 4731 | AGTAGTGAGAGTCAACCTGGAGGCC | 5995 | GGCCTCCAGGTTGACTCTCACTACT |
| 4732 | GAGTAGTGAGAGTCAACCTGGAGGC | 5996 | GCCTCCAGGTTGACTCTCACTACTC |
| 4733 | TGAGTAGTGAGAGTCAACCTGGAGG | 5997 | CCTCCAGGTTGACTCTCACTACTCA |
| 4734 | ATGAGTAGTGAGAGTCAACCTGGAG | 5998 | CTCCAGGTTGACTCTCACTACTCAT |
| 4735 | GATGAGTAGTGAGAGTCAACCTGGA | 5999 | TCCAGGTTGACTCTCACTACTCATC |
| 4736 | TGATGAGTAGTGAGAGTCAACCTGG | 6000 | CCAGGTTGACTCTCACTACTCATCA |
| 4737 | ATGATGAGTAGTGAGAGTCAACCTG | 6001 | CAGGTTGACTCTCACTACTCATCAT |
| 4738 | GATGATGAGTAGTGAGAGTCAACCT | 6002 | AGGTTGACTCTCACTACTCATCATC |
| 4739 | TGATGATGAGTAGTGAGAGTCAACC | 6003 | GGTTGACTCTCACTACTCATCATCA |
| 4740 | CTGATGATGAGTAGTGAGAGTCAAC | 6004 | GTTGACTCTCACTACTCATCATCAG |
| 4741 | CCTGATGATGAGTAGTGAGAGTCAA | 6005 | TTGACTCTCACTACTCATCATCAGG |
| 4742 | ACCTGATGATGAGTAGTGAGAGTCA | 6006 | TGACTCTCACTACTCATCATCAGGT |
| 4743 | AACCTGATGATGAGTAGTGAGAGTC | 6007 | GACTCTCACTACTCATCATCAGGTT |
| 4744 | GAACCTGATGATGAGTAGTGAGAGT | 6008 | ACTCTCACTACTCATCATCAGGTTC |
| 4745 | AGAACCTGATGATGAGTAGTGAGAG | 6009 | CTCTCACTACTCATCATCAGGTTCT |
| 4746 | AAGAACCTGATGATGAGTAGTGAGA | 6010 | TCTCACTACTCATCATCAGGTTCTT |
| 4747 | GAAGAACCTGATGATGAGTAGTGAG | 6011 | CTCACTACTCATCATCAGGTTCTTC |
| 4748 | GGAAGAACCTGATGATGAGTAGTGA | 6012 | TCACTACTCATCATCAGGTTCTTCC |
| 4749 | AGGAAGAACCTGATGATGAGTAGTG | 6013 | CACTACTCATCATCAGGTTCTTCCT |
| 4750 | AAGGAAGAACCTGATGATGAGTAGT | 6014 | ACTACTCATCATCAGGTTCTTCCTT |
| 4751 | GAAGGAAGAACCTGATGATGAGTAG | 6015 | CTACTCATCATCAGGTTCTTCCTTC |
| 4752 | AGAAGGAAGAACCTGATGATGAGTA | 6016 | TACTCATCATCAGGTTCTTCCTTCT |
| 4753 | TAGAAGGAAGAACCTGATGATGAGT | 6017 | ACTCATCATCAGGTTCTTCCTTCTA |
| 4754 | ATAGAAGGAAGAACCTGATGATGAG | 6018 | CTCATCATCAGGTTCTTCCTTCTAT |
| 4755 | AATAGAAGGAAGAACCTGATGATGA | 6019 | TCATCATCAGGTTCTTCCTTCTATT |
| 4756 | GAATAGAAGGAAGAACCTGATGATG | 6020 | CATCATCAGGTTCTTCCTTCTATTC |
| 4757 | GGAATAGAAGGAAGAACCTGATGAT | 6021 | ATCATCAGGTTCTTCCTTCTATTCC |
| 4758 | TGGAATAGAAGGAAGAACCTGATGA | 6022 | TCATCAGGTTCTTCCTTCTATTCCA |
| 4759 | CTGGAATAGAAGGAAGAACCTGATG | 6023 | CATCAGGTTCTTCCTTCTATTCCAG |
| 4760 | GCTGGAATAGAAGGAAGAACCTGAT | 6024 | ATCAGGTTCTTCCTTCTATTCCAGC |
| 4761 | GGCTGGAATAGAAGGAAGAACCTGA | 6025 | TCAGGTTCTTCCTTCTATTCCAGCC |
| 4762 | GGGCTGGAATAGAAGGAAGAACCTG | 6026 | CAGGTTCTTCCTTCTATTCCAGCCC |
| 4763 | AGGGCTGGAATAGAAGGAAGAACCT | 6027 | AGGTTCTTCCTTCTATTCCAGCCCT |
| 4764 | TAGGGCTGGAATAGAAGGAAGAACC | 6028 | GGTTCTTCCTTCTATTCCAGCCCTA |
| 4765 | TTAGGGCTGGAATAGAAGGAAGAAC | 6029 | GTTCTTCCTTCTATTCCAGCCCTAA |
| 4766 | GTTAGGGCTGGAATAGAAGGAAGAA | 6030 | TTCTTCCTTCTATTCCAGCCCTAAC |
| 4767 | GGTTAGGGCTGGAATAGAAGGAAGA | 6031 | TCTTCCTTCTATTCCAGCCCTAACC |
| 4768 | TGGTTAGGGCTGGAATAGAAGGAAG | 6032 | CTTCCTTCTATTCCAGCCCTAACCA |
| 4769 | GTGGTTAGGGCTGGAATAGAAGGAA | 6033 | TTCCTTCTATTCCAGCCCTAACCAC |
| 4770 | AGTGGTTAGGGCTGGAATAGAAGGA | 6034 | TCCTTCTATTCCAGCCCTAACCACT |
| 4771 | GAGTGGTTAGGGCTGGAATAGAAGG | 6035 | CCTTCTATTCCAGCCCTAACCACTC |
| 4772 | TGAGTGGTTAGGGCTGGAATAGAAG | 6036 | CTTCTATTCCAGCCCTAACCACTCA |
| 4773 | CTGAGTGGTTAGGGCTGGAATAGAA | 6037 | TTCTATTCCAGCCCTAACCACTCAG |
| 4774 | CCTGAGTGGTTAGGGCTGGAATAGA | 6038 | TCTATTCCAGCCCTAACCACTCAGG |
| 4775 | TCCTGAGTGGTTAGGGCTGGAATAG | 6039 | CTATTCCAGCCCTAACCACTCAGGA |
| 4776 | ATCCTGAGTGGTTAGGGCTGGAATA | 6040 | TATTCCAGCCCTAACCACTCAGGAT |
| 4777 | AATCCTGAGTGGTTAGGGCTGGAAT | 6041 | ATTCCAGCCCTAACCACTCAGGATT |
| 4778 | CAATCCTGAGTGGTTAGGGCTGGAA | 6042 | TTCCAGCCCTAACCACTCAGGATTG |
| 4779 | CCAATCCTGAGTGGTTAGGGCTGGA | 6043 | TCCAGCCCTAACCACTCAGGATTGG |
| 4780 | CCCAATCCTGAGTGGTTAGGGCTGG | 6044 | CCAGCCCTAACCACTCAGGATTGGG |
| 4781 | GCCCAATCCTGAGTGGTTAGGGCTG | 6045 | CAGCCCTAACCACTCAGGATTGGGC |
| 4782 | GGCCCAATCCTGAGTGGTTAGGGCT | 6046 | AGCCCTAACCACTCAGGATTGGGCC |
| 4783 | CGGCCCAATCCTGAGTGGTTAGGGC | 6047 | GCCCTAACCACTCAGGATTGGGCCG |
| 4784 | ACGGCCCAATCCTGAGTGGTTAGGG | 6048 | CCCTAACCACTCAGGATTGGGCCGT |
| 4785 | AACGGCCCAATCCTGAGTGGTTAGG | 6049 | CCTAACCACTCAGGATTGGGCCGTT |
| 4786 | AAACGGCCCAATCCTGAGTGGTTAG | 6050 | CTAACCACTCAGGATTGGGCCGTTT |
| 4787 | CAAACGGCCCAATCCTGAGTGGTTA | 6051 | TAACCACTCAGGATTGGGCCGTTTG |
| 4788 | ACAAACGGCCCAATCCTGAGTGGTT | 6052 | AACCACTCAGGATTGGGCCGTTTGT |
| 4789 | CACAAACGGCCCAATCCTGAGTGGT | 6053 | ACCACTCAGGATTGGGCCGTTTGTG |
| 4790 | ACACAAACGGCCCAATCCTGAGTGG | 6054 | CCACTCAGGATTGGGCCGTTTGTGT |
| 4791 | GACACAAACGGCCCAATCCTGAGTG | 6055 | CACTCAGGATTGGGCCGTTTGTGTC |
| 4792 | AGACACAAACGGCCCAATCCTGAGT | 6056 | ACTCAGGATTGGGCCGTTTGTGTCT |
| 4793 | CAGACACAAACGGCCCAATCCTGAG | 6057 | CTCAGGATTGGGCCGTTTGTGTCTG |
| 4794 | CCAGACACAAACGGCCCAATCCTGA | 6058 | TCAGGATTGGGCCGTTTGTGTCTGG |
| 4795 | CCCAGACACAAACGGCCCAATCCTG | 6059 | CAGGATTGGGCCGTTTGTGTCTGGG |
| 4796 | ACCCAGACACAAACGGCCCAATCCT | 6060 | AGGATTGGGCCGTTTGTGTCTGGGT |
| 4797 | TACCCAGACACAAACGGCCCAATCC | 6061 | GGATTGGGCCGTTTGTGTCTGGGTA |
| 4798 | ATACCCAGACACAAACGGCCCAATC | 6062 | GATTGGGCCGTTTGTGTCTGGGTAT |
| 4799 | CATACCCAGACACAAACGGCCCAAT | 6063 | ATTGGGCCGTTTGTGTCTGGGTATG |
| 4800 | ACATACCCAGACACAAACGGCCCAA | 6064 | TTGGGCCGTTTGTGTCTGGGTATGT |
| 4801 | GACATACCCAGACACAAACGGCCCA | 6065 | TGGGCCGTTTGTGTCTGGGTATGTC |
| 4802 | AGACATACCCAGACACAAACGGCCC | 6066 | GGGCCGTTTGTGTCTGGGTATGTCT |
| 4803 | GAGACATACCCAGACACAAACGGCC | 6067 | GGCCGTTTGTGTCTGGGTATGTCTC |
| 4804 | AGAGACATACCCAGACACAAACGGC | 6068 | GCCGTTTGTGTCTGGGTATGTCTCT |
| 4805 | AAGAGACATACCCAGACACAAACGG | 6069 | CCGTTTGTGTCTGGGTATGTCTCTT |
| 4806 | GAAGAGACATACCCAGACACAAACG | 6070 | CGTTTGTGTCTGGGTATGTCTCTTC |
| 4807 | GGAAGAGACATACCCAGACACAAAC | 6071 | GTTTGTGTCTGGGTATGTCTCTTCC |
| 4808 | TGGAAGAGACATACCCAGACACAAA | 6072 | TTTGTGTCTGGGTATGTCTCTTCCA |
| 4809 | CTGGAAGAGACATACCCAGACACAA | 6073 | TTGTGTCTGGGTATGTCTCTTCCAG |
| 4810 | GCTGGAAGAGACATACCCAGACACA | 6074 | TGTGTCTGGGTATGTCTCTTCCAGC |
| 4811 | AGCTGGAAGAGACATACCCAGACAC | 6075 | GTGTCTGGGTATGTCTCTTCCAGCT |
| 4812 | CAGCTGGAAGAGACATACCCAGACA | 6076 | TGTCTGGGTATGTCTCTTCCAGCTG |
| 4813 | GCAGCTGGAAGAGACATACCCAGAC | 6077 | GTCTGGGTATGTCTCTTCCAGCTGC |
| 4814 | GGCAGCTGGAAGAGACATACCCAGA | 6078 | TCTGGGTATGTCTCTTCCAGCTGCC |
| 4815 | AGGCAGCTGGAAGAGACATACCCAG | 6079 | CTGGGTATGTCTCTTCCAGCTGCCT |
| 4816 | CAGGCAGCTGGAAGAGACATACCCA | 6080 | TGGGTATGTCTCTTCCAGCTGCCTG |
| 4817 | CCAGGCAGCTGGAAGAGACATACCC | 6081 | GGGTATGTCTCTTCCAGCTGCCTGG |
| 4818 | CCCAGGCAGCTGGAAGAGACATACC | 6082 | GGTATGTCTCTTCCAGCTGCCTGGG |
| 4819 | ACCCAGGCAGCTGGAAGAGACATAC | 6083 | GTATGTCTCTTCCAGCTGCCTGGGT |
| 4820 | AACCCAGGCAGCTGGAAGAGACATA | 6084 | TATGTCTCTTCCAGCTGCCTGGGTT |
| 4821 | AAACCCAGGCAGCTGGAAGAGACAT | 6085 | ATGTCTCTTCCAGCTGCCTGGGTTT |
| 4822 | GAAACCCAGGCAGCTGGAAGAGACA | 6086 | TGTCTCTTCCAGCTGCCTGGGTTTC |
| 4823 | GGAAACCCAGGCAGCTGGAAGAGAC | 6087 | GTCTCTTCCAGCTGCCTGGGTTTCC |
| 4824 | AGGAAACCCAGGCAGCTGGAAGAGA | 6088 | TCTCTTCCAGCTGCCTGGGTTTCCT |
| 4825 | CAGGAAACCCAGGCAGCTGGAAGAG | 6089 | CTCTTCCAGCTGCCTGGGTTTCCTG |
| 4826 | CCAGGAAACCCAGGCAGCTGGAAGA | 6090 | TCTTCCAGCTGCCTGGGTTTCCTGG |
| 4827 | TCCAGGAAACCCAGGCAGCTGGAAG | 6091 | CTTCCAGCTGCCTGGGTTTCCTGGA |
| 4828 | TTCCAGGAAACCCAGGCAGCTGGAA | 6092 | TTCCAGCTGCCTGGGTTTCCTGGAA |
| 4829 | TTTCCAGGAAACCCAGGCAGCTGGA | 6093 | TCCAGCTGCCTGGGTTTCCTGGAAA |
| 4830 | CTTTCCAGGAAACCCAGGCAGCTGG | 6094 | CCAGCTGCCTGGGTTTCCTGGAAAG |
| 4831 | TCTTTCCAGGAAACCCAGGCAGCTG | 6095 | CAGCTGCCTGGGTTTCCTGGAAAGA |
| 4832 | TTCTTTCCAGGAAACCCAGGCAGCT | 6096 | AGCTGCCTGGGTTTCCTGGAAAGAA |
| 4833 | GTTCTTTCCAGGAAACCCAGGCAGC | 6097 | GCTGCCTGGGTTTCCTGGAAAGAAC |
| 4834 | AGTTCTTTCCAGGAAACCCAGGCAG | 6098 | CTGCCTGGGTTTCCTGGAAAGAACT |
| 4835 | GAGTTCTTTCCAGGAAACCCAGGCA | 6099 | TGCCTGGGTTTCCTGGAAAGAACTC |
| 4836 | AGAGTTCTTTCCAGGAAACCCAGGC | 6100 | GCCTGGGTTTCCTGGAAAGAACTCT |
| 4837 | AAGAGTTCTTTCCAGGAAACCCAGG | 6101 | CCTGGGTTTCCTGGAAAGAACTCTT |
| 4838 | TAAGAGTTCTTTCCAGGAAACCCAG | 6102 | CTGGGTTTCCTGGAAAGAACTCTTA |
| 4839 | ATAAGAGTTCTTTCCAGGAAACCCA | 6103 | TGGGTTTCCTGGAAAGAACTCTTAT |
| 4840 | GATAAGAGTTCTTTCCAGGAAACCC | 6104 | GGGTTTCCTGGAAAGAACTCTTATC |
| 4841 | GGATAAGAGTTCTTTCCAGGAAACC | 6105 | GGTTTCCTGGAAAGAACTCTTATCC |
| 4842 | GGGATAAGAGTTCTTTCCAGGAAAC | 6106 | GTTTCCTGGAAAGAACTCTTATCCC |
| 4843 | GGGGATAAGAGTTCTTTCCAGGAAA | 6107 | TTTCCTGGAAAGAACTCTTATCCCC |
| 4844 | TGGGGATAAGAGTTCTTTCCAGGAA | 6108 | TTCCTGGAAAGAACTCTTATCCCCA |
| 4845 | CTGGGGATAAGAGTTCTTTCCAGGA | 6109 | TCCTGGAAAGAACTCTTATCCCCAG |
| 4846 | CCTGGGGATAAGAGTTCTTTCCAGG | 6110 | CCTGGAAAGAACTCTTATCCCCAGG |
| 4847 | TCCTGGGGATAAGAGTTCTTTCCAG | 6111 | CTGGAAAGAACTCTTATCCCCAGGA |
| 4848 | TTCCTGGGGATAAGAGTTCTTTCCA | 6112 | TGGAAAGAACTCTTATCCCCAGGAA |
| 4849 | GTTCCTGGGGATAAGAGTTCTTTCC | 6113 | GGAAAGAACTCTTATCCCCAGGAAC |
| 4850 | AGTTCCTGGGGATAAGAGTTCTTTC | 6114 | GAAAGAACTCTTATCCCCAGGAACT |
| 4851 | TAGTTCCTGGGGATAAGAGTTCTTT | 6115 | AAAGAACTCTTATCCCCAGGAACTA |
| 4852 | CTAGTTCCTGGGGATAAGAGTTCTT | 6116 | AAGAACTCTTATCCCCAGGAACTAG |
| 4853 | ACTAGTTCCTGGGGATAAGAGTTCT | 6117 | AGAACTCTTATCCCCAGGAACTAGT |
| 4854 | AACTAGTTCCTGGGGATAAGAGTTC | 6118 | GAACTCTTATCCCCAGGAACTAGTT |
| 4855 | AAACTAGTTCCTGGGGATAAGAGTT | 6119 | AACTCTTATCCCCAGGAACTAGTTT |
| 4856 | CAAACTAGTTCCTGGGGATAAGAGT | 6120 | ACTCTTATCCCCAGGAACTAGTTTG |
| 4857 | ACAAACTAGTTCCTGGGGATAAGAG | 6121 | CTCTTATCCCCAGGAACTAGTTTGT |
| 4858 | AACAAACTAGTTCCTGGGGATAAGA | 6122 | TCTTATCCCCAGGAACTAGTTTGTT |
| 4859 | CAACAAACTAGTTCCTGGGGATAAG | 6123 | CTTATCCCCAGGAACTAGTTTGTTG |
| 4860 | TCAACAAACTAGTTCCTGGGGATAA | 6124 | TTATCCCCAGGAACTAGTTTGTTGA |
| 4861 | TTCAACAAACTAGTTCCTGGGGATA | 6125 | TATCCCCAGGAACTAGTTTGTTGAA |
| 4862 | ATTCAACAAACTAGTTCCTGGGGAT | 6126 | ATCCCCAGGAACTAGTTTGTTGAAT |
| 4863 | TATTCAACAAACTAGTTCCTGGGGA | 6127 | TCCCCAGGAACTAGTTTGTTGAATA |
| 4864 | TTATTCAACAAACTAGTTCCTGGGG | 6128 | CCCCAGGAACTAGTTTGTTGAATAA |
| 4865 | TTTATTCAACAAACTAGTTCCTGGG | 6129 | CCCAGGAACTAGTTTGTTGAATAAA |
| 4866 | ATTTATTCAACAAACTAGTTCCTGG | 6130 | CCAGGAACTAGTTTGTTGAATAAAT |
| 4867 | CATTTATTCAACAAACTAGTTCCTG | 6131 | CAGGAACTAGTTTGTTGAATAAATG |
| 4868 | GCATTTATTCAACAAACTAGTTCCT | 6132 | AGGAACTAGTTTGTTGAATAAATGC |
| 4869 | AGCATTTATTCAACAAACTAGTTCC | 6133 | GGAACTAGTTTGTTGAATAAATGCT |
| 4870 | CAGCATTTATTCAACAAACTAGTTC | 6134 | GAACTAGTTTGTTGAATAAATGCTG |
| 4871 | CCAGCATTTATTCAACAAACTAGTT | 6135 | AACTAGTTTGTTGAATAAATGCTGG |
| 4872 | ACCAGCATTTATTCAACAAACTAGT | 6136 | ACTAGTTTGTTGAATAAATGCTGGT |
| 4873 | CACCAGCATTTATTCAACAAACTAG | 6137 | CTAGTTTGTTGAATAAATGCTGGTG |
| 4874 | TCACCAGCATTTATTCAACAAACTA | 6138 | TAGTTTGTTGAATAAATGCTGGTGA |
| 4875 | TTCACCAGCATTTATTCAACAAACT | 6139 | AGTTTGTTGAATAAATGCTGGTGAA |
| 4876 | ATTCACCAGCATTTATTCAACAAAC | 6140 | GTTTGTTGAATAAATGCTGGTGAAT |
| 4877 | CATTCACCAGCATTTATTCAACAAA | 6141 | TTTGTTGAATAAATGCTGGTGAATG |
| 4878 | TCATTCACCAGCATTTATTCAACAA | 6142 | TTGTTGAATAAATGCTGGTGAATGA |
| 4879 | TTCATTCACCAGCATTTATTCAACA | 6143 | TGTTGAATAAATGCTGGTGAATGAA |
| 4880 | ATTCATTCACCAGCATTTATTCAAC | 6144 | GTTGAATAAATGCTGGTGAATGAAT |
| 4881 | CATTCATTCACCAGCATTTATTCAA | 6145 | TTGAATAAATGCTGGTGAATGAATG |
| 4882 | TCATTCATTCACCAGCATTTATTCA | 6146 | TGAATAAATGCTGGTGAATGAATGA |
| 4883 | TTCATTCATTCACCAGCATTTATTC | 6147 | GAATAAATGCTGGTGAATGAATGAA |
| 4884 | ATTCATTCATTCACCAGCATTTATT | 6148 | AATAAATGCTGGTGAATGAATGAAT |
| 4885 | CATTCATTCATTCACCAGCATTTAT | 6149 | ATAAATGCTGGTGAATGAATGAATG |
| 4886 | TCATTCATTCATTCACCAGCATTTA | 6150 | TAAATGCTGGTGAATGAATGAATGA |
| 4887 | ATCATTCATTCATTCACCAGCATTT | 6151 | AAATGCTGGTGAATGAATGAATGAT |
| 4888 | AATCATTCATTCATTCACCAGCATT | 6152 | AATGCTGGTGAATGAATGAATGATT |
| 4889 | CAATCATTCATTCATTCACCAGCAT | 6153 | ATGCTGGTGAATGAATGAATGATTG |
| 4890 | TCAATCATTCATTCATTCACCAGCA | 6154 | TGCTGGTGAATGAATGAATGATTGA |
| 4891 | TTCAATCATTCATTCATTCACCAGC | 6155 | GCTGGTGAATGAATGAATGATTGAA |
| 4892 | GTTCAATCATTCATTCATTCACCAG | 6156 | CTGGTGAATGAATGAATGATTGAAC |
| 4893 | TGTTCAATCATTCATTCATTCACCA | 6157 | TGGTGAATGAATGAATGATTGAACA |
| 4894 | CTGTTCAATCATTCATTCATTCACC | 6158 | GGTGAATGAATGAATGATTGAACAG |
| 4895 | TCTGTTCAATCATTCATTCATTCAC | 6159 | GTGAATGAATGAATGATTGAACAGA |
| 4896 | ATCTGTTCAATCATTCATTCATTCA | 6160 | TGAATGAATGAATGATTGAACAGAT |
| 4897 | CATCTGTTCAATCATTCATTCATTC | 6161 | GAATGAATGAATGATTGAACAGATG |
| 4898 | TCATCTGTTCAATCATTCATTCATT | 6162 | AATGAATGAATGATTGAACAGATGA |
| 4899 | TTCATCTGTTCAATCATTCATTCAT | 6163 | ATGAATGAATGATTGAACAGATGAA |
| 4900 | ATTCATCTGTTCAATCATTCATTCA | 6164 | TGAATGAATGATTGAACAGATGAAT |
| 4901 | CATTCATCTGTTCAATCATTCATTC | 6165 | GAATGAATGATTGAACAGATGAATG |
| 4902 | TCATTCATCTGTTCAATCATTCATT | 6166 | AATGAATGATTGAACAGATGAATGA |
| 4903 | CTCATTCATCTGTTCAATCATTCAT | 6167 | ATGAATGATTGAACAGATGAATGAG |
| 4904 | ACTCATTCATCTGTTCAATCATTCA | 6168 | TGAATGATTGAACAGATGAATGAGT |
| 4905 | CACTCATTCATCTGTTCAATCATTC | 6169 | GAATGATTGAACAGATGAATGAGTG |
| 4906 | TCACTCATTCATCTGTTCAATCATT | 6170 | AATGATTGAACAGATGAATGAGTGA |
| 4907 | ATCACTCATTCATCTGTTCAATCAT | 6171 | ATGATTGAACAGATGAATGAGTGAT |
| 4908 | CATCACTCATTCATCTGTTCAATCA | 6172 | TGATTGAACAGATGAATGAGTGATG |
| 4909 | TCATCACTCATTCATCTGTTCAATC | 6173 | GATTGAACAGATGAATGAGTGATGA |
| 4910 | CTCATCACTCATTCATCTGTTCAAT | 6174 | ATTGAACAGATGAATGAGTGATGAG |
| 4911 | ACTCATCACTCATTCATCTGTTCAA | 6175 | TTGAACAGATGAATGAGTGATGAGT |
| 4912 | TACTCATCACTCATTCATCTGTTCA | 6176 | TGAACAGATGAATGAGTGATGAGTA |
| 4913 | CTACTCATCACTCATTCATCTGTTC | 6177 | GAACAGATGAATGAGTGATGAGTAG |
| 4914 | TCTACTCATCACTCATTCATCTGTT | 6178 | AACAGATGAATGAGTGATGAGTAGA |
| 4915 | ATCTACTCATCACTCATTCATCTGT | 6179 | ACAGATGAATGAGTGATGAGTAGAT |
| 4916 | TATCTACTCATCACTCATTCATCTG | 6180 | CAGATGAATGAGTGATGAGTAGATA |
| 4917 | TTATCTACTCATCACTCATTCATCT | 6181 | AGATGAATGAGTGATGAGTAGATAA |
| 4918 | TTTATCTACTCATCACTCATTCATC | 6182 | GATGAATGAGTGATGAGTAGATAAA |
| 4919 | TTTTATCTACTCATCACTCATTCAT | 6183 | ATGAATGAGTGATGAGTAGATAAAA |
| 4920 | CTTTTATCTACTCATCACTCATTCA | 6184 | TGAATGAGTGATGAGTAGATAAAAG |
| 4921 | CCTTTTATCTACTCATCACTCATTC | 6185 | GAATGAGTGATGAGTAGATAAAAGG |
| 4922 | TCCTTTTATCTACTCATCACTCATT | 6186 | AATGAGTGATGAGTAGATAAAAGGA |
| 4923 | ATCCTTTTATCTACTCATCACTCAT | 6187 | ATGAGTGATGAGTAGATAAAAGGAT |
| 4924 | CATCCTTTTATCTACTCATCACTCA | 6188 | TGAGTGATGAGTAGATAAAAGGATG |
| 4925 | CCATCCTTTTATCTACTCATCACTC | 6189 | GAGTGATGAGTAGATAAAAGGATGG |
| 4926 | TCCATCCTTTTATCTACTCATCACT | 6190 | AGTGATGAGTAGATAAAAGGATGGA |
| 4927 | ATCCATCCTTTTATCTACTCATCAC | 6191 | GTGATGAGTAGATAAAAGGATGGAT |
| 4928 | CATCCATCCTTTTATCTACTCATCA | 6192 | TGATGAGTAGATAAAAGGATGGATG |
| 4929 | CCATCCATCCTTTTATCTACTCATC | 6193 | GATGAGTAGATAAAAGGATGGATGG |
| 4930 | TCCATCCATCCTTTTATCTACTCAT | 6194 | ATGAGTAGATAAAAGGATGGATGGA |
| 4931 | CTCCATCCATCCTTTTATCTACTCA | 6195 | TGAGTAGATAAAAGGATGGATGGAG |
| 4932 | TCTCCATCCATCCTTTTATCTACTC | 6196 | GAGTAGATAAAAGGATGGATGGAGA |
| 4933 | CTCTCCATCCATCCTTTTATCTACT | 6197 | AGTAGATAAAAGGATGGATGGAGAG |
| 4934 | TCTCTCCATCCATCCTTTTATCTAC | 6198 | GTAGATAAAAGGATGGATGGAGAGA |
| 4935 | ATCTCTCCATCCATCCTTTTATCTA | 6199 | TAGATAAAAGGATGGATGGAGAGAT |
| 4936 | CATCTCTCCATCCATCCTTTTATCT | 6200 | AGATAAAAGGATGGATGGAGAGATG |
| 4937 | CCATCTCTCCATCCATCCTTTTATC | 6201 | GATAAAAGGATGGATGGAGAGATGG |
| 4938 | CCCATCTCTCCATCCATCCTTTTAT | 6202 | ATAAAAGGATGGATGGAGAGATGGG |
| 4939 | ACCCATCTCTCCATCCATCCTTTTA | 6203 | TAAAAGGATGGATGGAGAGATGGGT |
| 4940 | CACCCATCTCTCCATCCATCCTTTT | 6204 | AAAAGGATGGATGGAGAGATGGGTG |
| 4941 | TCACCCATCTCTCCATCCATCCTTT | 6205 | AAAGGATGGATGGAGAGATGGGTGA |
| 4942 | CTCACCCATCTCTCCATCCATCCTT | 6206 | AAGGATGGATGGAGAGATGGGTGAG |
| 4943 | ACTCACCCATCTCTCCATCCATCCT | 6207 | AGGATGGATGGAGAGATGGGTGAGT |
| 4944 | TACTCACCCATCTCTCCATCCATCC | 6208 | GGATGGATGGAGAGATGGGTGAGTA |
| 4945 | GTACTCACCCATCTCTCCATCCATC | 6209 | GATGGATGGAGAGATGGGTGAGTAC |
| 4946 | TGTACTCACCCATCTCTCCATCCAT | 6210 | ATGGATGGAGAGATGGGTGAGTACA |
| 4947 | ATGTACTCACCCATCTCTCCATCCA | 6211 | TGGATGGAGAGATGGGTGAGTACAT |
| 4948 | CATGTACTCACCCATCTCTCCATCC | 6212 | GGATGGAGAGATGGGTGAGTACATG |
| 4949 | CCATGTACTCACCCATCTCTCCATC | 6213 | GATGGAGAGATGGGTGAGTACATGG |
| 4950 | TCCATGTACTCACCCATCTCTCCAT | 6214 | ATGGAGAGATGGGTGAGTACATGGA |
| 4951 | ATCCATGTACTCACCCATCTCTCCA | 6215 | TGGAGAGATGGGTGAGTACATGGAT |
| 4952 | CATCCATGTACTCACCCATCTCTCC | 6216 | GGAGAGATGGGTGAGTACATGGATG |
| 4953 | CCATCCATGTACTCACCCATCTCTC | 6217 | GAGAGATGGGTGAGTACATGGATGG |
| 4954 | TCCATCCATGTACTCACCCATCTCT | 6218 | AGAGATGGGTGAGTACATGGATGGA |
| 4955 | ATCCATCCATGTACTCACCCATCTC | 6219 | GAGATGGGTGAGTACATGGATGGAT |
| 4956 | TATCCATCCATGTACTCACCCATCT | 6220 | AGATGGGTGAGTACATGGATGGATA |
| 4957 | CTATCCATCCATGTACTCACCCATC | 6221 | GATGGGTGAGTACATGGATGGATAG |
| 4958 | TCTATCCATCCATGTACTCACCCAT | 6222 | ATGGGTGAGTACATGGATGGATAGA |
| 4959 | ATCTATCCATCCATGTACTCACCCA | 6223 | TGGGTGAGTACATGGATGGATAGAT |
| 4960 | CATCTATCCATCCATGTACTCACCC | 6224 | GGGTGAGTACATGGATGGATAGATG |
| 4961 | CCATCTATCCATCCATGTACTCACC | 6225 | GGTGAGTACATGGATGGATAGATGG |
| 4962 | TCCATCTATCCATCCATGTACTCAC | 6226 | GTGAGTACATGGATGGATAGATGGA |
| 4963 | ATCCATCTATCCATCCATGTACTCA | 6227 | TGAGTACATGGATGGATAGATGGAT |
| 4964 | CATCCATCTATCCATCCATGTACTC | 6228 | GAGTACATGGATGGATAGATGGATG |
| 4965 | TCATCCATCTATCCATCCATGTACT | 6229 | AGTACATGGATGGATAGATGGATGA |
| 4966 | CTCATCCATCTATCCATCCATGTAC | 6230 | GTACATGGATGGATAGATGGATGAG |
| 4967 | ACTCATCCATCTATCCATCCATGTA | 6231 | TACATGGATGGATAGATGGATGAGT |
| 4968 | AACTCATCCATCTATCCATCCATGT | 6232 | ACATGGATGGATAGATGGATGAGTT |
| 4969 | CAACTCATCCATCTATCCATCCATG | 6233 | CATGGATGGATAGATGGATGAGTTG |
| 4970 | CCAACTCATCCATCTATCCATCCAT | 6234 | ATGGATGGATAGATGGATGAGTTGG |
| 4971 | ACCAACTCATCCATCTATCCATCCA | 6235 | TGGATGGATAGATGGATGAGTTGGT |
| 4972 | CACCAACTCATCCATCTATCCATCC | 6236 | GGATGGATAGATGGATGAGTTGGTG |
| 4973 | CCACCAACTCATCCATCTATCCATC | 6237 | GATGGATAGATGGATGAGTTGGTGG |
| 4974 | CCCACCAACTCATCCATCTATCCAT | 6238 | ATGGATAGATGGATGAGTTGGTGGG |
| 4975 | ACCCACCAACTCATCCATCTATCCA | 6239 | TGGATAGATGGATGAGTTGGTGGGT |
| 4976 | TACCCACCAACTCATCCATCTATCC | 6240 | GGATAGATGGATGAGTTGGTGGGTA |
| 4977 | CTACCCACCAACTCATCCATCTATC | 6241 | GATAGATGGATGAGTTGGTGGGTAG |
| 4978 | TCTACCCACCAACTCATCCATCTAT | 6242 | ATAGATGGATGAGTTGGTGGGTAGA |
| 4979 | ATCTACCCACCAACTCATCCATCTA | 6243 | TAGATGGATGAGTTGGTGGGTAGAT |
| 4980 | AATCTACCCACCAACTCATCCATCT | 6244 | AGATGGATGAGTTGGTGGGTAGATT |
| 4981 | GAATCTACCCACCAACTCATCCATC | 6245 | GATGGATGAGTTGGTGGGTAGATTC |
| 4982 | CGAATCTACCCACCAACTCATCCAT | 6246 | ATGGATGAGTTGGTGGGTAGATTCG |
| 4983 | ACGAATCTACCCACCAACTCATCCA | 6247 | TGGATGAGTTGGTGGGTAGATTCGT |
| 4984 | CACGAATCTACCCACCAACTCATCC | 6248 | GGATGAGTTGGTGGGTAGATTCGTG |
| 4985 | CCACGAATCTACCCACCAACTCATC | 6249 | GATGAGTTGGTGGGTAGATTCGTGG |
| 4986 | GCCACGAATCTACCCACCAACTCAT | 6250 | ATGAGTTGGTGGGTAGATTCGTGGC |
| 4987 | AGCCACGAATCTACCCACCAACTCA | 6251 | TGAGTTGGTGGGTAGATTCGTGGCT |
| 4988 | TAGCCACGAATCTACCCACCAACTC | 6252 | GAGTTGGTGGGTAGATTCGTGGCTA |
| 4989 | CTAGCCACGAATCTACCCACCAACT | 6253 | AGTTGGTGGGTAGATTCGTGGCTAG |
| 4990 | TCTAGCCACGAATCTACCCACCAAC | 6254 | GTTGGTGGGTAGATTCGTGGCTAGA |
| 4991 | ATCTAGCCACGAATCTACCCACCAA | 6255 | TTGGTGGGTAGATTCGTGGCTAGAT |
| 4992 | CATCTAGCCACGAATCTACCCACCA | 6256 | TGGTGGGTAGATTCGTGGCTAGATG |
| 4993 | CCATCTAGCCACGAATCTACCCACC | 6257 | GGTGGGTAGATTCGTGGCTAGATGG |
| 4994 | TCCATCTAGCCACGAATCTACCCAC | 6258 | GTGGGTAGATTCGTGGCTAGATGGA |
| 4995 | ATCCATCTAGCCACGAATCTACCCA | 6259 | TGGGTAGATTCGTGGCTAGATGGAT |
| 4996 | CATCCATCTAGCCACGAATCTACCC | 6260 | GGGTAGATTCGTGGCTAGATGGATG |
| 4997 | TCATCCATCTAGCCACGAATCTACC | 6261 | GGTAGATTCGTGGCTAGATGGATGA |
| 4998 | ATCATCCATCTAGCCACGAATCTAC | 6262 | GTAGATTCGTGGCTAGATGGATGAT |
| 4999 | CATCATCCATCTAGCCACGAATCTA | 6263 | TAGATTCGTGGCTAGATGGATGATG |
| 5000 | CCATCATCCATCTAGCCACGAATCT | 6264 | AGATTCGTGGCTAGATGGATGATGG |
| 5001 | TCCATCATCCATCTAGCCACGAATC | 6265 | GATTCGTGGCTAGATGGATGATGGA |
| 5002 | ATCCATCATCCATCTAGCCACGAAT | 6266 | ATTCGTGGCTAGATGGATGATGGAT |
| 5003 | CATCCATCATCCATCTAGCCACGAA | 6267 | TTCGTGGCTAGATGGATGATGGATG |
| 5004 | CCATCCATCATCCATCTAGCCACGA | 6268 | TCGTGGCTAGATGGATGATGGATGG |
| 5005 | TCCATCCATCATCCATCTAGCCACG | 6269 | CGTGGCTAGATGGATGATGGATGGA |
| 5006 | ATCCATCCATCATCCATCTAGCCAC | 6270 | GTGGCTAGATGGATGATGGATGGAT |
| 5007 | CATCCATCCATCATCCATCTAGCCA | 6271 | TGGCTAGATGGATGATGGATGGATG |
| 5008 | CCATCCATCCATCATCCATCTAGCC | 6272 | GGCTAGATGGATGATGGATGGATGG |
| 5009 | TCCATCCATCCATCATCCATCTAGC | 6273 | GCTAGATGGATGATGGATGGATGGA |
| 5010 | GTCCATCCATCCATCATCCATCTAG | 6274 | CTAGATGGATGATGGATGGATGGAC |
| 5011 | TGTCCATCCATCCATCATCCATCTA | 6275 | TAGATGGATGATGGATGGATGGACA |
| 5012 | CTGTCCATCCATCCATCATCCATCT | 6276 | AGATGGATGATGGATGGATGGACAG |
| 5013 | TCTGTCCATCCATCCATCATCCATC | 6277 | GATGGATGATGGATGGATGGACAGA |
| 5014 | ATCTGTCCATCCATCCATCATCCAT | 6278 | ATGGATGATGGATGGATGGACAGAT |
| 5015 | CATCTGTCCATCCATCCATCATCCA | 6279 | TGGATGATGGATGGATGGACAGATG |
| 5016 | CCATCTGTCCATCCATCCATCATCC | 6280 | GGATGATGGATGGATGGACAGATGG |
| 5017 | TCCATCTGTCCATCCATCCATCATC | 6281 | GATGATGGATGGATGGACAGATGGA |
| 5018 | ATCCATCTGTCCATCCATCCATCAT | 6282 | ATGATGGATGGATGGACAGATGGAT |
| 5019 | CATCCATCTGTCCATCCATCCATCA | 6283 | TGATGGATGGATGGACAGATGGATG |
| 5020 | CCATCCATCTGTCCATCCATCCATC | 6284 | GATGGATGGATGGACAGATGGATGG |
| 5021 | TCCATCCATCTGTCCATCCATCCAT | 6285 | ATGGATGGATGGACAGATGGATGGA |
| 5022 | ATCCATCCATCTGTCCATCCATCCA | 6286 | TGGATGGATGGACAGATGGATGGAT |
| 5023 | TATCCATCCATCTGTCCATCCATCC | 6287 | GGATGGATGGACAGATGGATGGATA |
| 5024 | ATATCCATCCATCTGTCCATCCATC | 6288 | GATGGATGGACAGATGGATGGATAT |
| 5025 | TATATCCATCCATCTGTCCATCCAT | 6289 | ATGGATGGACAGATGGATGGATATA |
| 5026 | ATATATCCATCCATCTGTCCATCCA | 6290 | TGGATGGACAGATGGATGGATATAT |
| 5027 | CATATATCCATCCATCTGTCCATCC | 6291 | GGATGGACAGATGGATGGATATATG |
| 5028 | TCATATATCCATCCATCTGTCCATC | 6292 | GATGGACAGATGGATGGATATATGA |
| 5029 | ATCATATATCCATCCATCTGTCCAT | 6293 | ATGGACAGATGGATGGATATATGAT |
| 5030 | AATCATATATCCATCCATCTGTCCA | 6294 | TGGACAGATGGATGGATATATGATT |
| 5031 | CAATCATATATCCATCCATCTGTCC | 6295 | GGACAGATGGATGGATATATGATTG |
| 5032 | TCAATCATATATCCATCCATCTGTC | 6296 | GACAGATGGATGGATATATGATTGA |
| 5033 | TTCAATCATATATCCATCCATCTGT | 6297 | ACAGATGGATGGATATATGATTGAA |
| 5034 | GTTCAATCATATATCCATCCATCTG | 6298 | CAGATGGATGGATATATGATTGAAC |
| 5035 | AGTTCAATCATATATCCATCCATCT | 6299 | AGATGGATGGATATATGATTGAACT |
| 5036 | TAGTTCAATCATATATCCATCCATC | 6300 | GATGGATGGATATATGATTGAACTA |
| 5037 | ATAGTTCAATCATATATCCATCCAT | 6301 | ATGGATGGATATATGATTGAACTAT |
| 5038 | AATAGTTCAATCATATATCCATCCA | 6302 | TGGATGGATATATGATTGAACTATT |
| 5039 | CAATAGTTCAATCATATATCCATCC | 6303 | GGATGGATATATGATTGAACTATTG |
| 5040 | TCAATAGTTCAATCATATATCCATC | 6304 | GATGGATATATGATTGAACTATTGA |
| 5041 | TTCAATAGTTCAATCATATATCCAT | 6305 | ATGGATATATGATTGAACTATTGAA |
| 5042 | TTTCAATAGTTCAATCATATATCCA | 6306 | TGGATATATGATTGAACTATTGAAA |
| 5043 | CTTTCAATAGTTCAATCATATATCC | 6307 | GGATATATGATTGAACTATTGAAAG |
| 5044 | ACTTTCAATAGTTCAATCATATATO | 6308 | GATATATGATTGAACTATTGAAAGT |
| 5045 | TACTTTCAATAGTTCAATCATATAT | 6309 | ATATATGATTGAACTATTGAAAGTA |
| 5046 | ATACTTTCAATAGTTCAATCATATA | 6310 | TATATGATTGAACTATTGAAAGTAT |
| 5047 | TATACTTTCAATAGTTCAATCATAT | 6311 | ATATGATTGAACTATTGAAAGTATA |
| 5048 | CTATACTTTCAATAGTTCAATCATA | 6312 | TATGATTGAACTATTGAAAGTATAG |
| 5049 | TCTATACTTTCAATAGTTCAATCAT | 6313 | ATGATTGAACTATTGAAAGTATAGA |
| 5050 | ATCTATACTTTCAATAGTTCAATCA | 6314 | TGATTGAACTATTGAAAGTATAGAT |
| 5051 | CATCTATACTTTCAATAGTTCAATC | 6315 | GATTGAACTATTGAAAGTATAGATG |
| 5052 | ACATCTATACTTTCAATAGTTCAAT | 6316 | ATTGAACTATTGAAAGTATAGATGT |
| 5053 | TACATCTATACTTTCAATAGTTCAA | 6317 | TTGAACTATTGAAAGTATAGATGTA |
| 5054 | ATACATCTATACTTTCAATAGTTCA | 6318 | TGAACTATTGAAAGTATAGATGTAT |
| 5055 | CATACATCTATACTTTCAATAGTTC | 6319 | GAACTATTGAAAGTATAGATGTATG |
| 5056 | CCATACATCTATACTTTCAATAGTT | 6320 | AACTATTGAAAGTATAGATGTATGG |
| 5057 | TCCATACATCTATACTTTCAATAGT | 6321 | ACTATTGAAAGTATAGATGTATGGA |
| 5058 | ATCCATACATCTATACTTTCAATAG | 6322 | CTATTGAAAGTATAGATGTATGGAT |
| 5059 | CATCCATACATCTATACTTTCAATA | 6323 | TATTGAAAGTATAGATGTATGGATG |
| 5060 | CCATCCATACATCTATACTTTCAAT | 6324 | ATTGAAAGTATAGATGTATGGATGG |
| 5061 | CCCATCCATACATCTATACTTTCAA | 6325 | TTGAAAGTATAGATGTATGGATGGG |
| 5062 | ACCCATCCATACATCTATACTTTCA | 6326 | TGAAAGTATAGATGTATGGATGGGT |
| 5063 | CACCCATCCATACATCTATACTTTC | 6327 | GAAAGTATAGATGTATGGATGGGTG |
| 5064 | TCACCCATCCATACATCTATACTTT | 6328 | AAAGTATAGATGTATGGATGGGTGA |
| 5065 | TTCACCCATCCATACATCTATACTT | 6329 | AAGTATAGATGTATGGATGGGTGAA |
| 5066 | ATTCACCCATCCATACATCTATACT | 6330 | AGTATAGATGTATGGATGGGTGAAT |
| 5067 | AATTCACCCATCCATACATCTATAC | 6331 | GTATAGATGTATGGATGGGTGAATT |
| 5068 | AAATTCACCCATCCATACATCTATA | 6332 | TATAGATGTATGGATGGGTGAATTT |
| 5069 | CAAATTCACCCATCCATACATCTAT | 6333 | ATAGATGTATGGATGGGTGAATTTG |
| 5070 | CCAAATTCACCCATCCATACATCTA | 6334 | TAGATGTATGGATGGGTGAATTTGG |
| 5071 | CCCAAATTCACCCATCCATACATCT | 6335 | AGATGTATGGATGGGTGAATTTGGG |
| 5072 | CCCCAAATTCACCCATCCATACATC | 6336 | GATGTATGGATGGGTGAATTTGGGG |
| 5073 | CCCCCAAATTCACCCATCCATACAT | 6337 | ATGTATGGATGGGTGAATTTGGGGG |
| 5074 | ACCCCCAAATTCACCCATCCATACA | 6338 | TGTATGGATGGGTGAATTTGGGGGT |
| 5075 | TACCCCCAAATTCACCCATCCATAC | 6339 | GTATGGATGGGTGAATTTGGGGGTA |
| 5076 | TTACCCCCAAATTCACCCATCCATA | 6340 | TATGGATGGGTGAATTTGGGGGTAA |
| 5077 | ATTACCCCCAAATTCACCCATCCAT | 6341 | ATGGATGGGTGAATTTGGGGGTAAT |
| 5078 | AATTACCCCCAAATTCACCCATCCA | 6342 | TGGATGGGTGAATTTGGGGGTAATT |
| 5079 | CAATTACCCCCAAATTCACCCATCC | 6343 | GGATGGGTGAATTTGGGGGTAATTG |
| 5080 | ACAATTACCCCCAAATTCACCCATC | 6344 | GATGGGTGAATTTGGGGGTAATTGT |
| 5081 | AACAATTACCCCCAAATTCACCCAT | 6345 | ATGGGTGAATTTGGGGGTAATTGTT |
| 5082 | TAACAATTACCCCCAAATTCACCCA | 6346 | TGGGTGAATTTGGGGGTAATTGTTA |
| 5083 | CTAACAATTACCCCCAAATTCACCC | 6347 | GGGTGAATTTGGGGGTAATTGTTAG |
| 5084 | TCTAACAATTACCCCCAAATTCACC | 6348 | GGTGAATTTGGGGGTAATTGTTAGA |
| 5085 | ATCTAACAATTACCCCCAAATTCAC | 6349 | GTGAATTTGGGGGTAATTGTTAGAT |
| 5086 | CATCTAACAATTACCCCCAAATTCA | 6350 | TGAATTTGGGGGTAATTGTTAGATG |
| 5087 | TCATCTAACAATTACCCCCAAATTC | 6351 | GAATTTGGGGGTAATTGTTAGATGA |
| 5088 | ATCATCTAACAATTACCCCCAAATT | 6352 | AATTTGGGGGTAATTGTTAGATGAT |
| 5089 | CATCATCTAACAATTACCCCCAAAT | 6353 | ATTTGGGGGTAATTGTTAGATGATG |
| 5090 | CCATCATCTAACAATTACCCCCAAA | 6354 | TTTGGGGGTAATTGTTAGATGATGG |
| 5091 | TCCATCATCTAACAATTACCCCCAA | 6355 | TTGGGGGTAATTGTTAGATGATGGA |
| 5092 | ATCCATCATCTAACAATTACCCCCA | 6356 | TGGGGGTAATTGTTAGATGATGGAT |
| 5093 | CATCCATCATCTAACAATTACCCCC | 6357 | GGGGGTAATTGTTAGATGATGGATG |
| 5094 | TCATCCATCATCTAACAATTACCCC | 6358 | GGGGTAATTGTTAGATGATGGATGA |
| 5095 | CTCATCCATCATCTAACAATTACCC | 6359 | GGGTAATTGTTAGATGATGGATGAG |
| 5096 | ACTCATCCATCATCTAACAATTACC | 6360 | GGTAATTGTTAGATGATGGATGAGT |
| 5097 | TACTCATCCATCATCTAACAATTAC | 6361 | GTAATTGTTAGATGATGGATGAGTA |
| 5098 | ATACTCATCCATCATCTAACAATTA | 6362 | TAATTGTTAGATGATGGATGAGTAT |
| 5099 | TATACTCATCCATCATCTAACAATT | 6363 | AATTGTTAGATGATGGATGAGTATA |
| 5100 | CTATACTCATCCATCATCTAACAAT | 6364 | ATTGTTAGATGATGGATGAGTATAG |
| 5101 | TCTATACTCATCCATCATCTAACAA | 6365 | TTGTTAGATGATGGATGAGTATAGA |
| 5102 | ATCTATACTCATCCATCATCTAACA | 6366 | TGTTAGATGATGGATGAGTATAGAT |
| 5103 | CATCTATACTCATCCATCATCTAAC | 6367 | GTTAGATGATGGATGAGTATAGATG |
| 5104 | TCATCTATACTCATCCATCATCTAA | 6368 | TTAGATGATGGATGAGTATAGATGA |
| 5105 | TTCATCTATACTCATCCATCATCTA | 6369 | TAGATGATGGATGAGTATAGATGAA |
| 5106 | ATTCATCTATACTCATCCATCATCT | 6370 | AGATGATGGATGAGTATAGATGAAT |
| 5107 | CATTCATCTATACTCATCCATCATC | 6371 | GATGATGGATGAGTATAGATGAATG |
| 5108 | TCATTCATCTATACTCATCCATCAT | 6372 | ATGATGGATGAGTATAGATGAATGA |
| 5109 | ATCATTCATCTATACTCATCCATCA | 6373 | TGATGGATGAGTATAGATGAATGAT |
| 5110 | CATCATTCATCTATACTCATCCATC | 6374 | GATGGATGAGTATAGATGAATGATG |
| 5111 | CCATCATTCATCTATACTCATCCAT | 6375 | ATGGATGAGTATAGATGAATGATGG |
| 5112 | TCCATCATTCATCTATACTCATCCA | 6376 | TGGATGAGTATAGATGAATGATGGA |
| 5113 | ATCCATCATTCATCTATACTCATCC | 6377 | GGATGAGTATAGATGAATGATGGAT |
| 5114 | CATCCATCATTCATCTATACTCATC | 6378 | GATGAGTATAGATGAATGATGGATG |
| 5115 | CCATCCATCATTCATCTATACTCAT | 6379 | ATGAGTATAGATGAATGATGGATGG |
| 5116 | TCCATCCATCATTCATCTATACTCA | 6380 | TGAGTATAGATGAATGATGGATGGA |
| 5117 | ATCCATCCATCATTCATCTATACTC | 6381 | GAGTATAGATGAATGATGGATGGAT |
| 5118 | TATCCATCCATCATTCATCTATACT | 6382 | AGTATAGATGAATGATGGATGGATA |
| 5119 | TTATCCATCCATCATTCATCTATAC | 6383 | GTATAGATGAATGATGGATGGATAA |
| 5120 | GTTATCCATCCATCATTCATCTATA | 6384 | TATAGATGAATGATGGATGGATAAC |
| 5121 | AGTTATCCATCCATCATTCATCTAT | 6385 | ATAGATGAATGATGGATGGATAACT |
| 5122 | AAGTTATCCATCCATCATTCATCTA | 6386 | TAGATGAATGATGGATGGATAACTT |
| 5123 | CAAGTTATCCATCCATCATTCATCT | 6387 | AGATGAATGATGGATGGATAACTTG |
| 5124 | TCAAGTTATCCATCCATCATTCATC | 6388 | GATGAATGATGGATGGATAACTTGA |
| 5125 | ATCAAGTTATCCATCCATCATTCAT | 6389 | ATGAATGATGGATGGATAACTTGAT |
| 5126 | CATCAAGTTATCCATCCATCATTCA | 6390 | TGAATGATGGATGGATAACTTGATG |
| 5127 | TCATCAAGTTATCCATCCATCATTC | 6391 | GAATGATGGATGGATAACTTGATGA |
| 5128 | CTCATCAAGTTATCCATCCATCATT | 6392 | AATGATGGATGGATAACTTGATGAG |
| 5129 | ACTCATCAAGTTATCCATCCATCAT | 6393 | ATGATGGATGGATAACTTGATGAGT |
| 5130 | CACTCATCAAGTTATCCATCCATCA | 6394 | TGATGGATGGATAACTTGATGAGTG |
| 5131 | CCACTCATCAAGTTATCCATCCATC | 6395 | GATGGATGGATAACTTGATGAGTGG |
| 5132 | TCCACTCATCAAGTTATCCATCCAT | 6396 | ATGGATGGATAACTTGATGAGTGGA |
| 5133 | ATCCACTCATCAAGTTATCCATCCA | 6397 | TGGATGGATAACTTGATGAGTGGAT |
| 5134 | TATCCACTCATCAAGTTATCCATCC | 6398 | GGATGGATAACTTGATGAGTGGATA |
| 5135 | CTATCCACTCATCAAGTTATCCATC | 6399 | GATGGATAACTTGATGAGTGGATAG |
| 5136 | TCTATCCACTCATCAAGTTATCCAT | 6400 | ATGGATAACTTGATGAGTGGATAGA |
| 5137 | ATCTATCCACTCATCAAGTTATCCA | 6401 | TGGATAACTTGATGAGTGGATAGAT |
| 5138 | TATCTATCCACTCATCAAGTTATCC | 6402 | GGATAACTTGATGAGTGGATAGATA |
| 5139 | CTATCTATCCACTCATCAAGTTATC | 6403 | GATAACTTGATGAGTGGATAGATAG |
| 5140 | TCTATCTATCCACTCATCAAGTTAT | 6404 | ATAACTTGATGAGTGGATAGATAGA |
| 5141 | ATCTATCTATCCACTCATCAAGTTA | 6405 | TAACTTGATGAGTGGATAGATAGAT |
| 5142 | AATCTATCTATCCACTCATCAAGTT | 6406 | AACTTGATGAGTGGATAGATAGATT |
| 5143 | CAATCTATCTATCCACTCATCAAGT | 6407 | ACTTGATGAGTGGATAGATAGATTG |
| 5144 | GCAATCTATCTATCCACTCATCAAG | 6408 | CTTGATGAGTGGATAGATAGATTGC |
| 5145 | AGCAATCTATCTATCCACTCATCAA | 6409 | TTGATGAGTGGATAGATAGATTGCT |
| 5146 | CAGCAATCTATCTATCCACTCATCA | 6410 | TGATGAGTGGATAGATAGATTGCTG |
| 5147 | CCAGCAATCTATCTATCCACTCATC | 6411 | GATGAGTGGATAGATAGATTGCTGG |
| 5148 | TCCAGCAATCTATCTATCCACTCAT | 6412 | ATGAGTGGATAGATAGATTGCTGGA |
| 5149 | ATCCAGCAATCTATCTATCCACTCA | 6413 | TGAGTGGATAGATAGATTGCTGGAT |
| 5150 | TATCCAGCAATCTATCTATCCACTC | 6414 | GAGTGGATAGATAGATTGCTGGATA |
| 5151 | CTATCCAGCAATCTATCTATCCACT | 6415 | AGTGGATAGATAGATTGCTGGATAG |
| 5152 | TCTATCCAGCAATCTATCTATCCAC | 6416 | GTGGATAGATAGATTGCTGGATAGA |
| 5153 | ATCTATCCAGCAATCTATCTATCCA | 6417 | TGGATAGATAGATTGCTGGATAGAT |
| 5154 | CATCTATCCAGCAATCTATCTATCC | 6418 | GGATAGATAGATTGCTGGATAGATG |
| 5155 | TCATCTATCCAGCAATCTATCTATC | 6419 | GATAGATAGATTGCTGGATAGATGA |
| 5156 | ATCATCTATCCAGCAATCTATCTAT | 6420 | ATAGATAGATTGCTGGATAGATGAT |
| 5157 | AATCATCTATCCAGCAATCTATCTA | 6421 | TAGATAGATTGCTGGATAGATGATT |
| 5158 | CAATCATCTATCCAGCAATCTATCT | 6422 | AGATAGATTGCTGGATAGATGATTG |
| 5159 | TCAATCATCTATCCAGCAATCTATC | 6423 | GATAGATTGCTGGATAGATGATTGA |
| 5160 | GTCAATCATCTATCCAGCAATCTAT | 6424 | ATAGATTGCTGGATAGATGATTGAC |
| 5161 | AGTCAATCATCTATCCAGCAATCTA | 6425 | TAGATTGCTGGATAGATGATTGACT |
| 5162 | CAGTCAATCATCTATCCAGCAATCT | 6426 | AGATTGCTGGATAGATGATTGACTG |
| 5163 | CCAGTCAATCATCTATCCAGCAATC | 6427 | GATTGCTGGATAGATGATTGACTGG |
| 5164 | CCCAGTCAATCATCTATCCAGCAAT | 6428 | ATTGCTGGATAGATGATTGACTGGG |
| 5165 | ACCCAGTCAATCATCTATCCAGCAA | 6429 | TTGCTGGATAGATGATTGACTGGGT |
| 5166 | CACCCAGTCAATCATCTATCCAGCA | 6430 | TGCTGGATAGATGATTGACTGGGTG |
| 5167 | CCACCCAGTCAATCATCTATCCAGC | 6431 | GCTGGATAGATGATTGACTGGGTGG |
| 5168 | TCCACCCAGTCAATCATCTATCCAG | 6432 | CTGGATAGATGATTGACTGGGTGGA |
| 5169 | ATCCACCCAGTCAATCATCTATCCA | 6433 | TGGATAGATGATTGACTGGGTGGAT |
| 5170 | TATCCACCCAGTCAATCATCTATCC | 6434 | GGATAGATGATTGACTGGGTGGATA |
| 5171 | CTATCCACCCAGTCAATCATCTATC | 6435 | GATAGATGATTGACTGGGTGGATAG |
| 5172 | TCTATCCACCCAGTCAATCATCTAT | 6436 | ATAGATGATTGACTGGGTGGATAGA |
| 5173 | ATCTATCCACCCAGTCAATCATCTA | 6437 | TAGATGATTGACTGGGTGGATAGAT |
| 5174 | CATCTATCCACCCAGTCAATCATCT | 6438 | AGATGATTGACTGGGTGGATAGATG |
| 5175 | TCATCTATCCACCCAGTCAATCATC | 6439 | GATGATTGACTGGGTGGATAGATGA |
| 5176 | TTCATCTATCCACCCAGTCAATCAT | 6440 | ATGATTGACTGGGTGGATAGATGAA |
| 5177 | TTTCATCTATCCACCCAGTCAATCA | 6441 | TGATTGACTGGGTGGATAGATGAAA |
| 5178 | ATTTCATCTATCCACCCAGTCAATC | 6442 | GATTGACTGGGTGGATAGATGAAAT |
| 5179 | CATTTCATCTATCCACCCAGTCAAT | 6443 | ATTGACTGGGTGGATAGATGAAATG |
| 5180 | ACATTTCATCTATCCACCCAGTCAA | 6444 | TTGACTGGGTGGATAGATGAAATGT |
| 5181 | AACATTTCATCTATCCACCCAGTCA | 6445 | TGACTGGGTGGATAGATGAAATGTT |
| 5182 | CAACATTTCATCTATCCACCCAGTC | 6446 | GACTGGGTGGATAGATGAAATGTTG |
| 5183 | CCAACATTTCATCTATCCACCCAGT | 6447 | ACTGGGTGGATAGATGAAATGTTGG |
| 5184 | TCCAACATTTCATCTATCCACCCAG | 6448 | CTGGGTGGATAGATGAAATGTTGGA |
| 5185 | ATCCAACATTTCATCTATCCACCCA | 6449 | TGGGTGGATAGATGAAATGTTGGAT |
| 5186 | CATCCAACATTTCATCTATCCACCC | 6450 | GGGTGGATAGATGAAATGTTGGATG |
| 5187 | TCATCCAACATTTCATCTATCCACC | 6451 | GGTGGATAGATGAAATGTTGGATGA |
| 5188 | CTCATCCAACATTTCATCTATCCAC | 6452 | GTGGATAGATGAAATGTTGGATGAG |
| 5189 | GCTCATCCAACATTTCATCTATCCA | 6453 | TGGATAGATGAAATGTTGGATGAGC |
| 5190 | TGCTCATCCAACATTTCATCTATCC | 6454 | GGATAGATGAAATGTTGGATGAGCA |
| 5191 | CTGCTCATCCAACATTTCATCTATC | 6455 | GATAGATGAAATGTTGGATGAGCAG |
| 5192 | TCTGCTCATCCAACATTTCATCTAT | 6456 | ATAGATGAAATGTTGGATGAGCAGA |
| 5193 | ATCTGCTCATCCAACATTTCATCTA | 6457 | TAGATGAAATGTTGGATGAGCAGAT |
| 5194 | AATCTGCTCATCCAACATTTCATCT | 6458 | AGATGAAATGTTGGATGAGCAGATT |
| 5195 | TAATCTGCTCATCCAACATTTCATC | 6459 | GATGAAATGTTGGATGAGCAGATTA |
| 5196 | TTAATCTGCTCATCCAACATTTCAT | 6460 | ATGAAATGTTGGATGAGCAGATTAA |
| 5197 | CTTAATCTGCTCATCCAACATTTCA | 6461 | TGAAATGTTGGATGAGCAGATTAAG |
| 5198 | ACTTAATCTGCTCATCCAACATTTC | 6462 | GAAATGTTGGATGAGCAGATTAAGT |
| 5199 | AACTTAATCTGCTCATCCAACATTT | 6463 | AAATGTTGGATGAGCAGATTAAGTT |
| 5200 | CAACTTAATCTGCTCATCCAACATT | 6464 | AATGTTGGATGAGCAGATTAAGTTG |
| 5201 | ACAACTTAATCTGCTCATCCAACAT | 6465 | ATGTTGGATGAGCAGATTAAGTTGT |
| 5202 | TACAACTTAATCTGCTCATCCAACA | 6466 | TGTTGGATGAGCAGATTAAGTTGTA |
| 5203 | ATACAACTTAATCTGCTCATCCAAC | 6467 | GTTGGATGAGCAGATTAAGTTGTAT |
| 5204 | AATACAACTTAATCTGCTCATCCAA | 6468 | TTGGATGAGCAGATTAAGTTGTATT |
| 5205 | CAATACAACTTAATCTGCTCATCCA | 6469 | TGGATGAGCAGATTAAGTTGTATTG |
| 5206 | CCAATACAACTTAATCTGCTCATCC | 6470 | GGATGAGCAGATTAAGTTGTATTGG |
| 5207 | TCCAATACAACTTAATCTGCTCATC | 6471 | GATGAGCAGATTAAGTTGTATTGGA |
| 5208 | ATCCAATACAACTTAATCTGCTCAT | 6472 | ATGAGCAGATTAAGTTGTATTGGAT |
| 5209 | CATCCAATACAACTTAATCTGCTCA | 6473 | TGAGCAGATTAAGTTGTATTGGATG |
| 5210 | CCATCCAATACAACTTAATCTGCTC | 6474 | GAGCAGATTAAGTIGTATTGGATGG |
| 5211 | CCCATCCAATACAACTTAATCTGCT | 6475 | AGCAGATTAAGTTGTATTGGATGGG |
| 5212 | TCCCATCCAATACAACTTAATCTGC | 6476 | GCAGATTAAGTTGTATTGGATGGGA |
| 5213 | ATCCCATCCAATACAACTTAATCTG | 6477 | CAGATTAAGTTGTATTGGATGGGAT |
| 5214 | CATCCCATCCAATACAACTTAATCT | 6478 | AGATTAAGTIGTATTGGATGGGATG |
| 5215 | CCATCCCATCCAATACAACTTAATC | 6479 | GATTAAGTTGTATTGGATGGGATGG |
| 5216 | TCCATCCCATCCAATACAACTTAAT | 6480 | ATTAAGTTGTATTGGATGGGATGGA |
| 5217 | ATCCATCCCATCCAATACAACTTAA | 6481 | TTAAGTTGTATTGGATGGGATGGAT |
| 5218 | CATCCATCCCATCCAATACAACTTA | 6482 | TAAGTTGTATTGGATGGGATGGATG |
| 5219 | CCATCCATCCCATCCAATACAACTT | 6483 | AAGTTGTATTGGATGGGATGGATGG |
| 5220 | TCCATCCATCCCATCCAATACAACT | 6484 | AGTTGTATTGGATGGGATGGATGGA |
| 5221 | TTCCATCCATCCCATCCAATACAAC | 6485 | GTTGTATTGGATGGGATGGATGGAA |
| 5222 | CTTCCATCCATCCCATCCAATACAA | 6486 | TTGTATTGGATGGGATGGATGGAAG |
| 5223 | ACTTCCATCCATCCCATCCAATACA | 6487 | TGTATTGGATGGGATGGATGGAAGT |
| 5224 | CACTTCCATCCATCCCATCCAATAC | 6488 | GTATTGGATGGGATGGATGGAAGTG |
| 5225 | ACACTTCCATCCATCCCATCCAATA | 6489 | TATTGGATGGGATGGATGGAAGTGT |
| 5226 | CACACTTCCATCCATCCCATCCAAT | 6490 | ATTGGATGGGATGGATGGAAGTGTG |
| 5227 | CCACACTTCCATCCATCCCATCCAA | 6491 | TTGGATGGGATGGATGGAAGTGTGG |
| 5228 | ACCACACTTCCATCCATCCCATCCA | 6492 | TGGATGGGATGGATGGAAGTGTGGT |
| 5229 | AACCACACTTCCATCCATCCCATCC | 6493 | GGATGGGATGGATGGAAGTGTGGTT |
| 5230 | CAACCACACTTCCATCCATCCCATC | 6494 | GATGGGATGGATGGAAGTGTGGTTG |
| 5231 | TCAACCACACTTCCATCCATCCCAT | 6495 | ATGGGATGGATGGAAGTGTGGTTGA |
| 5232 | CTCAACCACACTTCCATCCATCCCA | 6496 | TGGGATGGATGGAAGTGTGGTTGAG |
| 5233 | ACTCAACCACACTTCCATCCATCCC | 6497 | GGGATGGATGGAAGTGTGGTTGAGT |
| 5234 | AACTCAACCACACTTCCATCCATCC | 6498 | GGATGGATGGAAGTGTGGTTGAGTT |
| 5235 | TAACTCAACCACACTTCCATCCATC | 6499 | GATGGATGGAAGTGTGGTTGAGTTA |
| 5236 | ATAACTCAACCACACTTCCATCCAT | 6500 | ATGGATGGAAGTGTGGTTGAGTTAT |
| 5237 | AATAACTCAACCACACTTCCATCCA | 6501 | TGGATGGAAGTGTGGTTGAGTTATT |
| 5238 | TAATAACTCAACCACACTTCCATCC | 6502 | GGATGGAAGTGTGGTTGAGTTATTA |
| 5239 | CTAATAACTCAACCACACTTCCATC | 6503 | GATGGAAGTGTGGTTGAGTTATTAG |
| 5240 | TCTAATAACTCAACCACACTTCCAT | 6504 | ATGGAAGTGTGGTTGAGTTATTAGA |
| 5241 | TTCTAATAACTCAACCACACTTCCA | 6505 | TGGAAGTGTGGTTGAGTTATTAGAA |
| 5242 | CTTCTAATAACTCAACCACACTTCC | 6506 | GGAAGTGTGGTTGAGTTATTAGAAG |
| 5243 | CCTTCTAATAACTCAACCACACTTC | 6507 | GAAGTGTGGTTGAGTTATTAGAAGG |
| 5244 | TCCTTCTAATAACTCAACCACACTT | 6508 | AAGTGTGGTTGAGTTATTAGAAGGA |
| 5245 | TTCCTTCTAATAACTCAACCACACT | 6509 | AGTGTGGTTGAGTTATTAGAAGGAA |
| 5246 | CTTCCTTCTAATAACTCAACCACAC | 6510 | GTGTGGTTGAGTTATTAGAAGGAAG |
| 5247 | TCTTCCTTCTAATAACTCAACCACA | 6511 | TGTGGTTGAGTTATTAGAAGGAAGA |
| 5248 | ATCTTCCTTCTAATAACTCAACCAC | 6512 | GTGGTTGAGTTATTAGAAGGAAGAT |
| 5249 | AATCTTCCTTCTAATAACTCAACCA | 6513 | TGGTTGAGTTATTAGAAGGAAGATT |
| 5250 | CAATCTTCCTTCTAATAACTCAACC | 6514 | GGTTGAGTTATTAGAAGGAAGATTG |
| 5251 | TCAATCTTCCTTCTAATAACTCAAC | 6515 | GTTGAGTTATTAGAAGGAAGATTGA |
| 5252 | CTCAATCTTCCTTCTAATAACTCAA | 6516 | TTGAGTTATTAGAAGGAAGATTGAG |
| 5253 | ACTCAATCTTCCTTCTAATAACTCA | 6517 | TGAGTTATTAGAAGGAAGATTGAGT |
| 5254 | TACTCAATCTTCCTTCTAATAACTC | 6518 | GAGTTATTAGAAGGAAGATTGAGTA |
| 5255 | CTACTCAATCTTCCTTCTAATAACT | 6519 | AGTTATTAGAAGGAAGATTGAGTAG |
| 5256 | TCTACTCAATCTTCCTTCTAATAAC | 6520 | GTTATTAGAAGGAAGATTGAGTAGA |
| 5257 | ATCTACTCAATCTTCCTTCTAATAA | 6521 | TTATTAGAAGGAAGATTGAGTAGAT |
| 5258 | TATCTACTCAATCTTCCTTCTAATA | 6522 | TATTAGAAGGAAGATTGAGTAGATA |
| 5259 | CTATCTACTCAATCTTCCTTCTAAT | 6523 | ATTAGAAGGAAGATTGAGTAGATAG |
| 5260 | CCTATCTACTCAATCTTCCTTCTAA | 6524 | TTAGAAGGAAGATTGAGTAGATAGG |
| 5261 | ACCTATCTACTCAATCTTCCTTCTA | 6525 | TAGAAGGAAGATTGAGTAGATAGGT |
| 5262 | CACCTATCTACTCAATCTTCCTTCT | 6526 | AGAAGGAAGATTGAGTAGATAGGTG |
| 5263 | TCACCTATCTACTCAATCTTCCTTC | 6527 | GAAGGAAGATTGAGTAGATAGGTGA |
| 5264 | TTCACCTATCTACTCAATCTTCCTT | 6528 | AAGGAAGATTGAGTAGATAGGTGAA |
| 5265 | ATTCACCTATCTACTCAATCTTCCT | 6529 | AGGAAGATTGAGTAGATAGGTGAAT |
| 5266 | AATTCACCTATCTACTCAATCTTCC | 6530 | GGAAGATTGAGTAGATAGGTGAATT |
| 5267 | AAATTCACCTATCTACTCAATCTTC | 6531 | GAAGATTGAGTAGATAGGTGAATTT |
| 5268 | CAAATTCACCTATCTACTCAATCTT | 6532 | AAGATTGAGTAGATAGGTGAATTTG |
| 5269 | ACAAATTCACCTATCTACTCAATCT | 6533 | AGATTGAGTAGATAGGTGAATTTGT |
| 5270 | AACAAATTCACCTATCTACTCAATC | 6534 | GATTGAGTAGATAGGTGAATTTGTT |
| 5271 | CAACAAATTCACCTATCTACTCAAT | 6535 | ATTGAGTAGATAGGTGAATTTGTTG |
| 5272 | TCAACAAATTCACCTATCTACTCAA | 6536 | TTGAGTAGATAGGTGAATTTGTTGA |
| 5273 | ATCAACAAATTCACCTATCTACTCA | 6537 | TGAGTAGATAGGTGAATTTGTTGAT |
| 5274 | TATCAACAAATTCACCTATCTACTC | 6538 | GAGTAGATAGGTGAATTTGTTGATA |
| 5275 | CTATCAACAAATTCACCTATCTACT | 6539 | AGTAGATAGGTGAATTTGTTGATAG |
| 5276 | ACTATCAACAAATTCACCTATCTAC | 6540 | GTAGATAGGTGAATTTGTTGATAGT |
| 5277 | GACTATCAACAAATTCACCTATCTA | 6541 | TAGATAGGTGAATTTGTTGATAGTC |
| 5278 | TGACTATCAACAAATTCACCTATCT | 6542 | AGATAGGTGAATTTGTTGATAGTCA |
| 5279 | CTGACTATCAACAAATTCACCTATC | 6543 | GATAGGTGAATTTGTTGATAGTCAG |
| 5280 | TCTGACTATCAACAAATTCACCTAT | 6544 | ATAGGTGAATTTGTTGATAGTCAGA |
| 5281 | ATCTGACTATCAACAAATTCACCTA | 6545 | TAGGTGAATTTGTTGATAGTCAGAT |
| 5282 | CATCTGACTATCAACAAATTCACCT | 6546 | AGGTGAATTTGTTGATAGTCAGATG |
| 5283 | CCATCTGACTATCAACAAATTCACC | 6547 | GGTGAATTTGTTGATAGTCAGATGG |
| 5284 | CCCATCTGACTATCAACAAATTCAC | 6548 | GTGAATTTGTTGATAGTCAGATGGG |
| 5285 | ACCCATCTGACTATCAACAAATTCA | 6549 | TGAATTTGTTGATAGTCAGATGGGT |
| 5286 | TACCCATCTGACTATCAACAAATTC | 6550 | GAATTTGTTGATAGTCAGATGGGTA |
| 5287 | CTACCCATCTGACTATCAACAAATT | 6551 | AATTTGTTGATAGTCAGATGGGTAG |
| 5288 | TCTACCCATCTGACTATCAACAAAT | 6552 | ATTTGTTGATAGTCAGATGGGTAGA |
| 5289 | ATCTACCCATCTGACTATCAACAAA | 6553 | TTTGTTGATAGTCAGATGGGTAGAT |
| 5290 | TATCTACCCATCTGACTATCAACAA | 6554 | TTGTTGATAGTCAGATGGGTAGATA |
| 5291 | CTATCTACCCATCTGACTATCAACA | 6555 | TGTTGATAGTCAGATGGGTAGATAG |
| 5292 | CCTATCTACCCATCTGACTATCAAC | 6556 | GTTGATAGTCAGATGGGTAGATAGG |
| 5293 | ACCTATCTACCCATCTGACTATCAA | 6557 | TTGATAGTCAGATGGGTAGATAGGT |
| 5294 | TACCTATCTACCCATCTGACTATCA | 6558 | TGATAGTCAGATGGGTAGATAGGTA |
| 5295 | CTACCTATCTACCCATCTGACTATC | 6559 | GATAGTCAGATGGGTAGATAGGTAG |
| 5296 | TCTACCTATCTACCCATCTGACTAT | 6560 | ATAGTCAGATGGGTAGATAGGTAGA |
| 5297 | ATCTACCTATCTACCCATCTGACTA | 6561 | TAGTCAGATGGGTAGATAGGTAGAT |
| 5298 | CATCTACCTATCTACCCATCTGACT | 6562 | AGTCAGATGGGTAGATAGGTAGATG |
| 5299 | CCATCTACCTATCTACCCATCTGAC | 6563 | GTCAGATGGGTAGATAGGTAGATGG |
| 5300 | TCCATCTACCTATCTACCCATCTGA | 6564 | TCAGATGGGTAGATAGGTAGATGGA |
| 5301 | ATCCATCTACCTATCTACCCATCTG | 6565 | CAGATGGGTAGATAGGTAGATGGAT |
| 5302 | CATCCATCTACCTATCTACCCATCT | 6566 | AGATGGGTAGATAGGTAGATGGATG |
| 5303 | CCATCCATCTACCTATCTACCCATC | 6567 | GATGGGTAGATAGGTAGATGGATGG |
| 5304 | TCCATCCATCTACCTATCTACCCAT | 6568 | ATGGGTAGATAGGTAGATGGATGGA |
| 5305 | ATCCATCCATCTACCTATCTACCCA | 6569 | TGGGTAGATAGGTAGATGGATGGAT |
| 5306 | CATCCATCCATCTACCTATCTACCC | 6570 | GGGTAGATAGGTAGATGGATGGATG |
| 5307 | CCATCCATCCATCTACCTATCTACC | 6571 | GGTAGATAGGTAGATGGATGGATGG |
| 5308 | TCCATCCATCCATCTACCTATCTAC | 6572 | GTAGATAGGTAGATGGATGGATGGA |
| 5309 | ATCCATCCATCCATCTACCTATCTA | 6573 | TAGATAGGTAGATGGATGGATGGAT |
| 5310 | CATCCATCCATCCATCTACCTATCT | 6574 | AGATAGGTAGATGGATGGATGGATG |
| 5311 | CCATCCATCCATCCATCTACCTATC | 6575 | GATAGGTAGATGGATGGATGGATGG |
| 5312 | TCCATCCATCCATCCATCTACCTAT | 6576 | ATAGGTAGATGGATGGATGGATGGA |
| 5313 | ATCCATCCATCCATCCATCTACCTA | 6577 | TAGGTAGATGGATGGATGGATGGAT |
| 5314 | CATCCATCCATCCATCCATCTACCT | 6578 | AGGTAGATGGATGGATGGATGGATG |
| 5315 | CCATCCATCCATCCATCCATCTACC | 6579 | GGTAGATGGATGGATGGATGGATGG |
| 5316 | TCCATCCATCCATCCATCCATCTAC | 6580 | GTAGATGGATGGATGGATGGATGGA |
| 5317 | ATCCATCCATCCATCCATCCATCTA | 6581 | TAGATGGATGGATGGATGGATGGAT |
| 5318 | CATCCATCCATCCATCCATCCATCT | 6582 | AGATGGATGGATGGATGGATGGATG |
| 5319 | ACATCCATCCATCCATCCATCCATC | 6583 | GATGGATGGATGGATGGATGGATGT |
| 5320 | TACATCCATCCATCCATCCATCCAT | 6584 | ATGGATGGATGGATGGATGGATGTA |
| 5321 | ATACATCCATCCATCCATCCATCCA | 6585 | TGGATGGATGGATGGATGGATGTAT |
| 5322 | TATACATCCATCCATCCATCCATCC | 6586 | GGATGGATGGATGGATGGATGTATA |
| 5323 | CTATACATCCATCCATCCATCCATC | 6587 | GATGGATGGATGGATGGATGTATAG |
| 5324 | CCTATACATCCATCCATCCATCCAT | 6588 | ATGGATGGATGGATGGATGTATAGG |
| 5325 | GCCTATACATCCATCCATCCATCCA | 6589 | TGGATGGATGGATGGATGTATAGGC |
| 5326 | TGCCTATACATCCATCCATCCATCC | 6590 | GGATGGATGGATGGATGTATAGGCA |
| 5327 | CTGCCTATACATCCATCCATCCATC | 6591 | GATGGATGGATGGATGTATAGGCAG |
| 5328 | TCTGCCTATACATCCATCCATCCAT | 6592 | ATGGATGGATGGATGTATAGGCAGA |
| 5329 | ATCTGCCTATACATCCATCCATCCA | 6593 | TGGATGGATGGATGTATAGGCAGAT |
| 5330 | CATCTGCCTATACATCCATCCATCC | 6594 | GGATGGATGGATGTATAGGCAGATG |
| 5331 | CCATCTGCCTATACATCCATCCATC | 6595 | GATGGATGGATGTATAGGCAGATGG |
| 5332 | TCCATCTGCCTATACATCCATCCAT | 6596 | ATGGATGGATGTATAGGCAGATGGA |
| 5333 | GTCCATCTGCCTATACATCCATCCA | 6597 | TGGATGGATGTATAGGCAGATGGAC |
| 5334 | TGTCCATCTGCCTATACATCCATCC | 6598 | GGATGGATGTATAGGCAGATGGACA |
| 5335 | TTGTCCATCTGCCTATACATCCATC | 6599 | GATGGATGTATAGGCAGATGGACAA |
| 5336 | TTTGTCCATCTGCCTATACATCCAT | 6600 | ATGGATGTATAGGCAGATGGACAAA |
| 5337 | ATTTGTCCATCTGCCTATACATCCA | 6601 | TGGATGTATAGGCAGATGGACAAAT |
| 5338 | CATTTGTCCATCTGCCTATACATCC | 6602 | GGATGTATAGGCAGATGGACAAATG |
| 5339 | CCATTTGTCCATCTGCCTATACATC | 6603 | GATGTATAGGCAGATGGACAAATGG |
| 5340 | TCCATTTGTCCATCTGCCTATACAT | 6604 | ATGTATAGGCAGATGGACAAATGGA |
| 5341 | ATCCATTTGTCCATCTGCCTATACA | 6605 | TGTATAGGCAGATGGACAAATGGAT |
| 5342 | CATCCATTTGTCCATCTGCCTATAC | 6606 | GTATAGGCAGATGGACAAATGGATG |
| 5343 | TCATCCATTTGTCCATCTGCCTATA | 6607 | TATAGGCAGATGGACAAATGGATGA |
| 5344 | TTCATCCATTTGTCCATCTGCCTAT | 6608 | ATAGGCAGATGGACAAATGGATGAA |
| 5345 | ATTCATCCATTTGTCCATCTGCCTA | 6609 | TAGGCAGATGGACAAATGGATGAAT |
| 5346 | CATTCATCCATTTGTCCATCTGCCT | 6610 | AGGCAGATGGACAAATGGATGAATG |
| 5347 | CCATTCATCCATTTGTCCATCTGCC | 6611 | GGCAGATGGACAAATGGATGAATGG |
| 5348 | CCCATTCATCCATTTGTCCATCTGC | 6612 | GCAGATGGACAAATGGATGAATGGG |
| 5349 | ACCCATTCATCCATTTGTCCATCTG | 6613 | CAGATGGACAAATGGATGAATGGGT |
| 5350 | CACCCATTCATCCATTTGTCCATCT | 6614 | AGATGGACAAATGGATGAATGGGTG |
| 5351 | CCACCCATTCATCCATTTGTCCATC | 6615 | GATGGACAAATGGATGAATGGGTGG |
| 5352 | CCCACCCATTCATCCATTTGTCCAT | 6616 | ATGGACAAATGGATGAATGGGTGGG |
| 5353 | ACCCACCCATTCATCCATTTGTCCA | 6617 | TGGACAAATGGATGAATGGGTGGGT |
| 5354 | CACCCACCCATTCATCCATTTGTCC | 6618 | GGACAAATGGATGAATGGGTGGGTG |
| 5355 | CCACCCACCCATTCATCCATTTGTC | 6619 | GACAAATGGATGAATGGGTGGGTGG |
| 5356 | TCCACCCACCCATTCATCCATTTGT | 6620 | ACAAATGGATGAATGGGTGGGTGGA |
| 5357 | ATCCACCCACCCATTCATCCATTTG | 6621 | CAAATGGATGAATGGGTGGGTGGAT |
| 5358 | CATCCACCCACCCATTCATCCATTT | 6622 | AAATGGATGAATGGGTGGGTGGATG |
| 5359 | TCATCCACCCACCCATTCATCCATT | 6623 | AATGGATGAATGGGTGGGTGGATGA |
| 5360 | TTCATCCACCCACCCATTCATCCAT | 6624 | ATGGATGAATGGGTGGGTGGATGAA |
| 5361 | ATTCATCCACCCACCCATTCATCCA | 6625 | TGGATGAATGGGTGGGTGGATGAAT |
| 5362 | CATTCATCCACCCACCCATTCATCC | 6626 | GGATGAATGGGTGGGTGGATGAATG |
| 5363 | CCATTCATCCACCCACCCATTCATC | 6627 | GATGAATGGGTGGGTGGATGAATGG |
| 5364 | TCCATTCATCCACCCACCCATTCAT | 6628 | ATGAATGGGTGGGTGGATGAATGGA |
| 5365 | TTCCATTCATCCACCCACCCATTCA | 6629 | TGAATGGGTGGGTGGATGAATGGAA |
| 5366 | CTTCCATTCATCCACCCACCCATTC | 6630 | GAATGGGTGGGTGGATGAATGGAAG |
| 5367 | CCTTCCATTCATCCACCCACCCATT | 6631 | AATGGGTGGGTGGATGAATGGAAGG |
| 5368 | TCCTTCCATTCATCCACCCACCCAT | 6632 | ATGGGTGGGTGGATGAATGGAAGGA |
| 5369 | ATCCTTCCATTCATCCACCCACCCA | 6633 | TGGGTGGGTGGATGAATGGAAGGAT |
| 5370 | CATCCTTCCATTCATCCACCCACCC | 6634 | GGGTGGGTGGATGAATGGAAGGATG |
| 5371 | ACATCCTTCCATTCATCCACCCACC | 6635 | GGTGGGTGGATGAATGGAAGGATGT |
| 5372 | CACATCCTTCCATTCATCCACCCAC | 6636 | GTGGGTGGATGAATGGAAGGATGTG |
| 5373 | ACACATCCTTCCATTCATCCACCCA | 6637 | TGGGTGGATGAATGGAAGGATGTGT |
| 5374 | CACACATCCTTCCATTCATCCACCC | 6638 | GGGTGGATGAATGGAAGGATGTGTG |
| 5375 | CCACACATCCTTCCATTCATCCACC | 6639 | GGTGGATGAATGGAAGGATGTGTGG |
| 5376 | ACCACACATCCTTCCATTCATCCAC | 6640 | GTGGATGAATGGAAGGATGTGTGGT |
| 5377 | AACCACACATCCTTCCATTCATCCA | 6641 | TGGATGAATGGAAGGATGTGTGGTT |
| 5378 | CAACCACACATCCTTCCATTCATCC | 6642 | GGATGAATGGAAGGATGTGTGGTTG |
| 5379 | TCAACCACACATCCTTCCATTCATC | 6643 | GATGAATGGAAGGATGTGTGGTTGA |
| 5380 | TTCAACCACACATCCTTCCATTCAT | 6644 | ATGAATGGAAGGATGTGTGGTTGAA |
| 5381 | GTTCAACCACACATCCTTCCATTCA | 6645 | TGAATGGAAGGATGTGTGGTTGAAC |
| 5382 | AGTTCAACCACACATCCTTCCATTC | 6646 | GAATGGAAGGATGTGTGGTTGAACT |
| 5383 | TAGTTCAACCACACATCCTTCCATT | 6647 | AATGGAAGGATGTGTGGTTGAACTA |
| 5384 | ATAGTTCAACCACACATCCTTCCAT | 6648 | ATGGAAGGATGTGTGGTTGAACTAT |
| 5385 | AATAGTTCAACCACACATCCTTCCA | 6649 | TGGAAGGATGTGTGGTTGAACTATT |
| 5386 | CAATAGTTCAACCACACATCCTTCC | 6650 | GGAAGGATGTGTGGTTGAACTATTG |
| 5387 | GCAATAGTTCAACCACACATCCTTC | 6651 | GAAGGATGTGTGGTTGAACTATTGC |
| 5388 | TGCAATAGTTCAACCACACATCCTT | 6652 | AAGGATGTGTGGTTGAACTATTGCA |
| 5389 | TTGCAATAGTTCAACCACACATCCT | 6653 | AGGATGTGTGGTTGAACTATTGCAA |
| 5390 | CTTGCAATAGTTCAACCACACATCC | 6654 | GGATGTGTGGTTGAACTATTGCAAG |
| 5391 | ACTTGCAATAGTTCAACCACACATC | 6655 | GATGTGTGGTTGAACTATTGCAAGT |
| 5392 | TACTTGCAATAGTTCAACCACACAT | 6656 | ATGTGTGGTTGAACTATTGCAAGTA |
| 5393 | ATACTTGCAATAGTTCAACCACACA | 6657 | TGTGTGGTTGAACTATTGCAAGTAT |
| 5394 | AATACTTGCAATAGTTCAACCACAC | 6658 | GTGTGGTTGAACTATTGCAAGTATT |
| 5395 | CAATACTTGCAATAGTTCAACCACA | 6659 | TGTGGTTGAACTATTGCAAGTATTG |
| 5396 | TCAATACTTGCAATAGTTCAACCAC | 6660 | GTGGTTGAACTATTGCAAGTATTGA |
| 5397 | ATCAATACTTGCAATAGTTCAACCA | 6661 | TGGTTGAACTATTGCAAGTATTGAT |
| 5398 | TATCAATACTTGCAATAGTTCAACC | 6662 | GGTTGAACTATTGCAAGTATTGATA |
| 5399 | TTATCAATACTTGCAATAGTTCAAC | 6663 | GTTGAACTATTGCAAGTATTGATAA |
| 5400 | ATTATCAATACTTGCAATAGTTCAA | 6664 | TTGAACTATTGCAAGTATTGATAAT |
| 5401 | AATTATCAATACTTGCAATAGTTCA | 6665 | TGAACTATTGCAAGTATTGATAATT |
| 5402 | CAATTATCAATACTTGCAATAGTTC | 6666 | GAACTATTGCAAGTATTGATAATTG |
| 5403 | CCAATTATCAATACTTGCAATAGTT | 6667 | AACTATTGCAAGTATTGATAATTGG |
| 5404 | CCCAATTATCAATACTTGCAATAGT | 6668 | ACTATTGCAAGTATTGATAATTGGG |
| 5405 | ACCCAATTATCAATACTTGCAATAG | 6669 | CTATTGCAAGTATTGATAATTGGGT |
| 5406 | AACCCAATTATCAATACTTGCAATA | 6670 | TATTGCAAGTATTGATAATTGGGTT |
| 5407 | GAACCCAATTATCAATACTTGCAAT | 6671 | ATTGCAAGTATTGATAATTGGGTTC |
| 5408 | TGAACCCAATTATCAATACTTGCAA | 6672 | TTGCAAGTATTGATAATTGGGTTCA |
| 5409 | ATGAACCCAATTATCAATACTTGCA | 6673 | TGCAAGTATTGATAATTGGGTTCAT |
| 5410 | TATGAACCCAATTATCAATACTTGC | 6674 | GCAAGTATTGATAATTGGGTTCATA |
| 5411 | TTATGAACCCAATTATCAATACTTG | 6675 | CAAGTATTGATAATTGGGTTCATAA |
| 5412 | ATTATGAACCCAATTATCAATACTT | 6676 | AAGTATTGATAATTGGGTTCATAAT |
| 5413 | AATTATGAACCCAATTATCAATACT | 6677 | AGTATTGATAATTGGGTTCATAATT |
| 5414 | AAATTATGAACCCAATTATCAATAC | 6678 | GTATTGATAATTGGGTTCATAATTT |
| 5415 | GAAATTATGAACCCAATTATCAATA | 6679 | TATTGATAATTGGGTTCATAATTTC |
| 5416 | AGAAATTATGAACCCAATTATCAAT | 6680 | ATTGATAATTGGGTTCATAATTTCT |
| 5417 | CAGAAATTATGAACCCAATTATCAA | 6681 | TTGATAATTGGGTTCATAATTTCTG |
| 5418 | TCAGAAATTATGAACCCAATTATCA | 6682 | TGATAATTGGGTTCATAATTTCTGA |
| 5419 | TTCAGAAATTATGAACCCAATTATC | 6683 | GATAATTGGGTTCATAATTTCTGAA |
| 5420 | ATTCAGAAATTATGAACCCAATTAT | 6684 | ATAATTGGGTTCATAATTTCTGAAT |
| 5421 | TATTCAGAAATTATGAACCCAATTA | 6685 | TAATTGGGTTCATAATTTCTGAATA |
| 5422 | ATATTCAGAAATTATGAACCCAATT | 6686 | AATTGGGTTCATAATTTCTGAATAT |
| 5423 | AATATTCAGAAATTATGAACCCAAT | 6687 | ATTGGGTTCATAATTTCTGAATATT |
| 5424 | AAATATTCAGAAATTATGAACCCAA | 6688 | TTGGGTTCATAATTTCTGAATATTT |
| 5425 | TAAATATTCAGAAATTATGAACCCA | 6689 | TGGGTTCATAATTTCTGAATATTTA |
| 5426 | CTAAATATTCAGAAATTATGAACCC | 6690 | GGGTTCATAATTTCTGAATATTTAG |
| 5427 | TCTAAATATTCAGAAATTATGAACC | 6691 | GGTTCATAATTTCTGAATATTTAGA |
| 5428 | ATCTAAATATTCAGAAATTATGAAC | 6692 | GTTCATAATTTCTGAATATTTAGAT |
| 5429 | CATCTAAATATTCAGAAATTATGAA | 6693 | TTCATAATTTCTGAATATTTAGATG |
| 5430 | CCATCTAAATATTCAGAAATTATGA | 6694 | TCATAATTTCTGAATATTTAGATGG |
| 5431 | TCCATCTAAATATTCAGAAATTATG | 6695 | CATAATTTCTGAATATTTAGATGGA |
| 5432 | ATCCATCTAAATATTCAGAAATTAT | 6696 | ATAATTTCTGAATATTTAGATGGAT |
| 5433 | CATCCATCTAAATATTCAGAAATTA | 6697 | TAATTTCTGAATATTTAGATGGATG |
| 5434 | CCATCCATCTAAATATTCAGAAATT | 6698 | AATTTCTGAATATTTAGATGGATGG |
| 5435 | ACCATCCATCTAAATATTCAGAAAT | 6699 | ATTTCTGAATATTTAGATGGATGGT |
| 5436 | AACCATCCATCTAAATATTCAGAAA | 6700 | TTTCTGAATATTTAGATGGATGGTT |
| 5437 | CAACCATCCATCTAAATATTCAGAA | 6701 | TTCTGAATATTTAGATGGATGGTTG |
| 5438 | ACAACCATCCATCTAAATATTCAGA | 6702 | TCTGAATATTTAGATGGATGGTTGT |
| 5439 | CACAACCATCCATCTAAATATTCAG | 6703 | CTGAATATTTAGATGGATGGTTGTG |
| 5440 | TCACAACCATCCATCTAAATATTCA | 6704 | TGAATATTTAGATGGATGGTTGTGA |
| 5441 | CTCACAACCATCCATCTAAATATTC | 6705 | GAATATTTAGATGGATGGTTGTGAG |
| 5442 | ACTCACAACCATCCATCTAAATATT | 6706 | AATATTTAGATGGATGGTTGTGAGT |
| 5443 | CACTCACAACCATCCATCTAAATAT | 6707 | ATATTTAGATGGATGGTTGTGAGTG |
| 5444 | CCACTCACAACCATCCATCTAAATA | 6708 | TATTTAGATGGATGGTTGTGAGTGG |
| 5445 | GCCACTCACAACCATCCATCTAAAT | 6709 | ATTTAGATGGATGGTTGTGAGTGGC |
| 5446 | AGCCACTCACAACCATCCATCTAAA | 6710 | TTTAGATGGATGGTTGTGAGTGGCT |
| 5447 | CAGCCACTCACAACCATCCATCTAA | 6711 | TTAGATGGATGGTTGTGAGTGGCTG |
| 5448 | CCAGCCACTCACAACCATCCATCTA | 6712 | TAGATGGATGGTTGTGAGTGGCTGG |
| 5449 | ACCAGCCACTCACAACCATCCATCT | 6713 | AGATGGATGGTTGTGAGTGGCTGGT |
| 5450 | CACCAGCCACTCACAACCATCCATC | 6714 | GATGGATGGTTGTGAGTGGCTGGTG |
| 5451 | CCACCAGCCACTCACAACCATCCAT | 6715 | ATGGATGGTTGTGAGTGGCTGGTGG |
| 5452 | TCCACCAGCCACTCACAACCATCCA | 6716 | TGGATGGTTGTGAGTGGCTGGTGGA |
| 5453 | GTCCACCAGCCACTCACAACCATCC | 6717 | GGATGGTTGTGAGTGGCTGGTGGAC |
| 5454 | TGTCCACCAGCCACTCACAACCATC | 6718 | GATGGTTGTGAGTGGCTGGTGGACA |
| 5455 | CTGTCCACCAGCCACTCACAACCAT | 6719 | ATGGTTGTGAGTGGCTGGTGGACAG |
| 5456 | TCTGTCCACCAGCCACTCACAACCA | 6720 | TGGTTGTGAGTGGCTGGTGGACAGA |
| 5457 | GTCTGTCCACCAGCCACTCACAACC | 6721 | GGTTGTGAGTGGCTGGTGGACAGAC |
| 5458 | CGTCTGTCCACCAGCCACTCACAAC | 6722 | GTTGTGAGTGGCTGGTGGACAGACG |
| 5459 | TCGTCTGTCCACCAGCCACTCACAA | 6723 | TTGTGAGTGGCTGGTGGACAGACGA |
| 5460 | TTCGTCTGTCCACCAGCCACTCACA | 6724 | TGTGAGTGGCTGGTGGACAGACGAA |
| 5461 | TTTCGTCTGTCCACCAGCCACTCAC | 6725 | GTGAGTGGCTGGTGGACAGACGAAA |
| 5462 | TTTTCGTCTGTCCACCAGCCACTCA | 6726 | TGAGTGGCTGGTGGACAGACGAAAA |
| 5463 | TTTTTCGTCTGTCCACCAGCCACTC | 6727 | GAGTGGCTGGTGGACAGACGAAAAA |
| 5464 | ATTTTTCGTCTGTCCACCAGCCACT | 6728 | AGTGGCTGGTGGACAGACGAAAAAT |
| 5465 | CATTTTTCGTCTGTCCACCAGCCAC | 6729 | GTGGCTGGTGGACAGACGAAAAATG |
| 5466 | CCATTTTTCGTCTGTCCACCAGCCA | 6730 | TGGCTGGTGGACAGACGAAAAATGG |
| 5467 | TCCATTTTTCGTCTGTCCACCAGCC | 6731 | GGCTGGTGGACAGACGAAAAATGGA |
| 5468 | ATCCATTTTTCGTCTGTCCACCAGC | 6732 | GCTGGTGGACAGACGAAAAATGGAT |
| 5469 | CATCCATTTTTCGTCTGTCCACCAG | 6733 | CTGGTGGACAGACGAAAAATGGATG |
| 5470 | CCATCCATTTTTCGTCTGTCCACCA | 6734 | TGGTGGACAGACGAAAAATGGATGG |
| 5471 | ACCATCCATTTTTCGTCTGTCCACC | 6735 | GGTGGACAGACGAAAAATGGATGGT |
| 5472 | AACCATCCATTTTTCGTCTGTCCAC | 6736 | GTGGACAGACGAAAAATGGATGGTT |
| 5473 | CAACCATCCATTTTTCGTCTGTCCA | 6737 | TGGACAGACGAAAAATGGATGGTTG |
| 5474 | CCAACCATCCATTTTTCGTCTGTCC | 6738 | GGACAGACGAAAAATGGATGGTTGG |
| 5475 | TCCAACCATCCATTTTTCGTCTGTC | 6739 | GACAGACGAAAAATGGATGGTTGGA |
| 5476 | ATCCAACCATCCATTTTTCGTCTGT | 6740 | ACAGACGAAAAATGGATGGTTGGAT |
| 5477 | TATCCAACCATCCATTTTTCGTCTG | 6741 | CAGACGAAAAATGGATGGTTGGATA |
| 5478 | TTATCCAACCATCCATTTTTCGTCT | 6742 | AGACGAAAAATGGATGGTTGGATAA |
| 5479 | TTTATCCAACCATCCATTTTTCGTC | 6743 | GACGAAAAATGGATGGTTGGATAAA |
| 5480 | ATTTATCCAACCATCCATTTTTCGT | 6744 | ACGAAAAATGGATGGTTGGATAAAT |
| 5481 | AATTTATCCAACCATCCATTTTTCG | 6745 | CGAAAAATGGATGGTTGGATAAATT |
| 5482 | CAATTTATCCAACCATCCATTTTTC | 6746 | GAAAAATGGATGGTTGGATAAATTG |
| 5483 | TCAATTTATCCAACCATCCATTTTT | 6747 | AAAAATGGATGGTTGGATAAATTGA |
| 5484 | ATCAATTTATCCAACCATCCATTTT | 6748 | AAAATGGATGGTTGGATAAATTGAT |
| 5485 | CATCAATTTATCCAACCATCCATTT | 6749 | AAATGGATGGTTGGATAAATTGATG |
| 5486 | CCATCAATTTATCCAACCATCCATT | 6750 | AATGGATGGTTGGATAAATTGATGG |
| 5487 | CCCATCAATTTATCCAACCATCCAT | 6751 | ATGGATGGTTGGATAAATTGATGGG |
| 5488 | ACCCATCAATTTATCCAACCATCCA | 6752 | TGGATGGTTGGATAAATTGATGGGT |
| 5489 | CACCCATCAATTTATCCAACCATCC | 6753 | GGATGGTTGGATAAATTGATGGGTG |
| 5490 | CCACCCATCAATTTATCCAACCATC | 6754 | GATGGTTGGATAAATTGATGGGTGG |
| 5491 | TCCACCCATCAATTTATCCAACCAT | 6755 | ATGGTTGGATAAATTGATGGGTGGA |
| 5492 | ATCCACCCATCAATTTATCCAACCA | 6756 | TGGTTGGATAAATTGATGGGTGGAT |
| 5493 | CATCCACCCATCAATTTATCCAACC | 6757 | GGTTGGATAAATTGATGGGTGGATG |
| 5494 | CCATCCACCCATCAATTTATCCAAC | 6758 | GTTGGATAAATTGATGGGTGGATGG |
| 5495 | TCCATCCACCCATCAATTTATCCAA | 6759 | TTGGATAAATTGATGGGTGGATGGA |
| 5496 | ATCCATCCACCCATCAATTTATCCA | 6760 | TGGATAAATTGATGGGTGGATGGAT |
| 5497 | CATCCATCCACCCATCAATTTATCC | 6761 | GGATAAATTGATGGGTGGATGGATG |
| 5498 | CCATCCATCCACCCATCAATTTATC | 6762 | GATAAATTGATGGGTGGATGGATGG |
| 5499 | ACCATCCATCCACCCATCAATTTAT | 6763 | ATAAATTGATGGGTGGATGGATGGT |
| 5500 | AACCATCCATCCACCCATCAATTTA | 6764 | TAAATTGATGGGTGGATGGATGGTT |
| 5501 | CAACCATCCATCCACCCATCAATTT | 6765 | AAATTGATGGGTGGATGGATGGTTG |
| 5502 | CCAACCATCCATCCACCCATCAATT | 6766 | AATTGATGGGTGGATGGATGGTTGG |
| 5503 | ACCAACCATCCATCCACCCATCAAT | 6767 | ATTGATGGGTGGATGGATGGTTGGT |
| 5504 | AACCAACCATCCATCCACCCATCAA | 6768 | TTGATGGGTGGATGGATGGTTGGTT |
| 5505 | CAACCAACCATCCATCCACCCATCA | 6769 | TGATGGGTGGATGGATGGTTGGTTG |
| 5506 | ACAACCAACCATCCATCCACCCATC | 6770 | GATGGGTGGATGGATGGTTGGTTGT |
| 5507 | TACAACCAACCATCCATCCACCCAT | 6771 | ATGGGTGGATGGATGGTTGGTTGTA |
| 5508 | ATACAACCAACCATCCATCCACCCA | 6772 | TGGGTGGATGGATGGTTGGTTGTAT |
| 5509 | CATACAACCAACCATCCATCCACCC | 6773 | GGGTGGATGGATGGTTGGTTGTATG |
| 5510 | TCATACAACCAACCATCCATCCACC | 6774 | GGTGGATGGATGGTTGGTTGTATGA |
| 5511 | TTCATACAACCAACCATCCATCCAC | 6775 | GTGGATGGATGGTTGGTTGTATGAA |
| 5512 | TTTCATACAACCAACCATCCATCCA | 6776 | TGGATGGATGGTTGGTTGTATGAAA |
| 5513 | CTTTCATACAACCAACCATCCATCC | 6777 | GGATGGATGGTTGGTTGTATGAAAG |
| 5514 | TCTTTCATACAACCAACCATCCATC | 6778 | GATGGATGGTTGGTTGTATGAAAGA |
| 5515 | TTCTTTCATACAACCAACCATCCAT | 6779 | ATGGATGGTTGGTTGTATGAAAGAA |
| 5516 | ATTCTTTCATACAACCAACCATCCA | 6780 | TGGATGGTTGGTTGTATGAAAGAAT |
| 5517 | CATTCTTTCATACAACCAACCATCC | 6781 | GGATGGTTGGTTGTATGAAAGAATG |
| 5518 | TCATTCTTTCATACAACCAACCATC | 6782 | GATGGTTGGTTGTATGAAAGAATGA |
| 5519 | TTCATTCTTTCATACAACCAACCAT | 6783 | ATGGTTGGTTGTATGAAAGAATGAA |
| 5520 | ATTCATTCTTTCATACAACCAACCA | 6784 | TGGTTGGTTGTATGAAAGAATGAAT |
| 5521 | CATTCATTCTTTCATACAACCAACC | 6785 | GGTTGGTTGTATGAAAGAATGAATG |
| 5522 | TCATTCATTCTTTCATACAACCAAC | 6786 | GTTGGTTGTATGAAAGAATGAATGA |
| 5523 | ATCATTCATTCTTTCATACAACCAA | 6787 | TTGGTTGTATGAAAGAATGAATGAT |
| 5524 | AATCATTCATTCTTTCATACAACCA | 6788 | TGGTTGTATGAAAGAATGAATGATT |
| 5525 | CAATCATTCATTCTTTCATACAACC | 6789 | GGTTGTATGAAAGAATGAATGATTG |
| 5526 | CCAATCATTCATTCTTTCATACAAC | 6790 | GTTGTATGAAAGAATGAATGATTGG |
| 5527 | CCCAATCATTCATTCTTTCATACAA | 6791 | TTGTATGAAAGAATGAATGATTGGG |
| 5528 | ACCCAATCATTCATTCTTTCATACA | 6792 | TGTATGAAAGAATGAATGATTGGGT |
| 5529 | TACCCAATCATTCATTCTTTCATAC | 6793 | GTATGAAAGAATGAATGATTGGGTA |
| 5530 | CTACCCAATCATTCATTCTTTCATA | 6794 | TATGAAAGAATGAATGATTGGGTAG |
| 5531 | CCTACCCAATCATTCATTCTTTCAT | 6795 | ATGAAAGAATGAATGATTGGGTAGG |
| 5532 | ACCTACCCAATCATTCATTCTTTCA | 6796 | TGAAAGAATGAATGATTGGGTAGGT |
| 5533 | CACCTACCCAATCATTCATTCTTTC | 6797 | GAAAGAATGAATGATTGGGTAGGTG |
| 5534 | CCACCTACCCAATCATTCATTCTTT | 6798 | AAAGAATGAATGATTGGGTAGGTGG |
| 5535 | TCCACCTACCCAATCATTCATTCTT | 6799 | AAGAATGAATGATTGGGTAGGTGGA |
| 5536 | ATCCACCTACCCAATCATTCATTCT | 6800 | AGAATGAATGATTGGGTAGGTGGAT |
| 5537 | AATCCACCTACCCAATCATTCATTC | 6801 | GAATGAATGATTGGGTAGGTGGATT |
| 5538 | TAATCCACCTACCCAATCATTCATT | 6802 | AATGAATGATTGGGTAGGTGGATTA |
| 5539 | TTAATCCACCTACCCAATCATTCAT | 6803 | ATGAATGATTGGGTAGGTGGATTAA |
| 5540 | CTTAATCCACCTACCCAATCATTCA | 6804 | TGAATGATTGGGTAGGTGGATTAAG |
| 5541 | ACTTAATCCACCTACCCAATCATTC | 6805 | GAATGATTGGGTAGGTGGATTAAGT |
| 5542 | AACTTAATCCACCTACCCAATCATT | 6806 | AATGATTGGGTAGGTGGATTAAGTT |
| 5543 | CAACTTAATCCACCTACCCAATCAT | 6807 | ATGATTGGGTAGGTGGATTAAGTTG |
| 5544 | GCAACTTAATCCACCTACCCAATCA | 6808 | TGATTGGGTAGGTGGATTAAGTTGC |
| 5545 | CGCAACTTAATCCACCTACCCAATC | 6809 | GATTGGGTAGGTGGATTAAGTTGCG |
| 5546 | CCGCAACTTAATCCACCTACCCAAT | 6810 | ATTGGGTAGGTGGATTAAGTTGCGG |
| 5547 | TCCGCAACTTAATCCACCTACCCAA | 6811 | TTGGGTAGGTGGATTAAGTTGCGGA |
| 5548 | ATCCGCAACTTAATCCACCTACCCA | 6812 | TGGGTAGGTGGATTAAGTTGCGGAT |
| 5549 | GATCCGCAACTTAATCCACCTACCC | 6813 | GGGTAGGTGGATTAAGTTGCGGATC |
| 5550 | TGATCCGCAACTTAATCCACCTACC | 6814 | GGTAGGTGGATTAAGTTGCGGATCA |
| 5551 | TTGATCCGCAACTTAATCCACCTAC | 6815 | GTAGGTGGATTAAGTTGCGGATCAA |
| 5552 | ATTGATCCGCAACTTAATCCACCTA | 6816 | TAGGTGGATTAAGTTGCGGATCAAT |
| 5553 | CATTGATCCGCAACTTAATCCACCT | 6817 | AGGTGGATTAAGTTGCGGATCAATG |
| 5554 | ACATTGATCCGCAACTTAATCCACC | 6818 | GGTGGATTAAGTTGCGGATCAATGT |
| 5555 | TACATTGATCCGCAACTTAATCCAC | 6819 | GTGGATTAAGTTGCGGATCAATGTA |
| 5556 | ATACATTGATCCGCAACTTAATCCA | 6820 | TGGATTAAGTTGCGGATCAATGTAT |
| 5557 | CATACATTGATCCGCAACTTAATCC | 6821 | GGATTAAGTTGCGGATCAATGTATG |
| 5558 | CCATACATTGATCCGCAACTTAATC | 6822 | GATTAAGTTGCGGATCAATGTATGG |
| 5559 | CCCATACATTGATCCGCAACTTAAT | 6823 | ATTAAGTTGCGGATCAATGTATGGG |
| 5560 | TCCCATACATTGATCCGCAACTTAA | 6824 | TTAAGTTGCGGATCAATGTATGGGA |
| 5561 | ATCCCATACATTGATCCGCAACTTA | 6825 | TAAGTTGCGGATCAATGTATGGGAT |
| 5562 | CATCCCATACATTGATCCGCAACTT | 6826 | AAGTTGCGGATCAATGTATGGGATG |
| 5563 | CCATCCCATACATTGATCCGCAACT | 6827 | AGTTGCGGATCAATGTATGGGATGG |
| 5564 | TCCATCCCATACATTGATCCGCAAC | 6828 | GTTGCGGATCAATGTATGGGATGGA |
| 5565 | ATCCATCCCATACATTGATCCGCAA | 6829 | TTGCGGATCAATGTATGGGATGGAT |
| 5566 | CATCCATCCCATACATTGATCCGCA | 6830 | TGCGGATCAATGTATGGGATGGATG |
| 5567 | TCATCCATCCCATACATTGATCCGC | 6831 | GCGGATCAATGTATGGGATGGATGA |
| 5568 | TTCATCCATCCCATACATTGATCCG | 6832 | CGGATCAATGTATGGGATGGATGAA |
| 5569 | ATTCATCCATCCCATACATTGATCC | 6833 | GGATCAATGTATGGGATGGATGAAT |
| 5570 | CATTCATCCATCCCATACATTGATC | 6834 | GATCAATGTATGGGATGGATGAATG |
| 5571 | CCATTCATCCATCCCATACATTGAT | 6835 | ATCAATGTATGGGATGGATGAATGG |
| 5572 | TCCATTCATCCATCCCATACATTGA | 6836 | TCAATGTATGGGATGGATGAATGGA |
| 5573 | ATCCATTCATCCATCCCATACATTG | 6837 | CAATGTATGGGATGGATGAATGGAT |
| 5574 | CATCCATTCATCCATCCCATACATT | 6838 | AATGTATGGGATGGATGAATGGATG |
| 5575 | CCATCCATTCATCCATCCCATACAT | 6839 | ATGTATGGGATGGATGAATGGATGG |
| 5576 | TCCATCCATTCATCCATCCCATACA | 6840 | TGTATGGGATGGATGAATGGATGGA |
| 5577 | ATCCATCCATTCATCCATCCCATAC | 6841 | GTATGGGATGGATGAATGGATGGAT |
| 5578 | CATCCATCCATTCATCCATCCCATA | 6842 | TATGGGATGGATGAATGGATGGATG |
| 5579 | CCATCCATCCATTCATCCATCCCAT | 6843 | ATGGGATGGATGAATGGATGGATGG |
| 5580 | TCCATCCATCCATTCATCCATCCCA | 6844 | TGGGATGGATGAATGGATGGATGGA |
| 5581 | ATCCATCCATCCATTCATCCATCCC | 6845 | GGGATGGATGAATGGATGGATGGAT |
| 5582 | CATCCATCCATCCATTCATCCATCC | 6846 | GGATGGATGAATGGATGGATGGATG |
| 5583 | CCATCCATCCATCCATTCATCCATC | 6847 | GATGGATGAATGGATGGATGGATGG |
| 5584 | TCCATCCATCCATCCATTCATCCAT | 6848 | ATGGATGAATGGATGGATGGATGGA |
| 5585 | ATCCATCCATCCATCCATTCATCCA | 6849 | TGGATGAATGGATGGATGGATGGAT |
| 5586 | CATCCATCCATCCATCCATTCATCC | 6850 | GGATGAATGGATGGATGGATGGATG |
| 5587 | ACATCCATCCATCCATCCATTCATC | 6851 | GATGAATGGATGGATGGATGGATGT |
| 5588 | CACATCCATCCATCCATCCATTCAT | 6852 | ATGAATGGATGGATGGATGGATGTG |
| 5589 | ACACATCCATCCATCCATCCATTCA | 6853 | TGAATGGATGGATGGATGGATGTGT |
| 5590 | CACACATCCATCCATCCATCCATTC | 6854 | GAATGGATGGATGGATGGATGTGTG |
| 5591 | CCACACATCCATCCATCCATCCATT | 6855 | AATGGATGGATGGATGGATGTGTGG |
| 5592 | ACCACACATCCATCCATCCATCCAT | 6856 | ATGGATGGATGGATGGATGTGTGGT |
| 5593 | AACCACACATCCATCCATCCATCCA | 6857 | TGGATGGATGGATGGATGTGTGGTT |
| 5594 | CAACCACACATCCATCCATCCATCC | 6858 | GGATGGATGGATGGATGTGTGGTTG |
| 5595 | TCAACCACACATCCATCCATCCATC | 6859 | GATGGATGGATGGATGTGTGGTTGA |
| 5596 | TTCAACCACACATCCATCCATCCAT | 6860 | ATGGATGGATGGATGTGTGGTTGAA |
| 5597 | ATTCAACCACACATCCATCCATCCA | 6861 | TGGATGGATGGATGTGTGGTTGAAT |
| 5598 | AATTCAACCACACATCCATCCATCC | 6862 | GGATGGATGGATGTGTGGTTGAATT |
| 5599 | TAATTCAACCACACATCCATCCATC | 6863 | GATGGATGGATGTGTGGTTGAATTA |
| 5600 | GTAATTCAACCACACATCCATCCAT | 6864 | ATGGATGGATGTGTGGTTGAATTAC |
| 5601 | AGTAATTCAACCACACATCCATCCA | 6865 | TGGATGGATGTGTGGTTGAATTACT |
| 5602 | CAGTAATTCAACCACACATCCATCC | 6866 | GGATGGATGTGTGGTTGAATTACTG |
| 5603 | TCAGTAATTCAACCACACATCCATC | 6867 | GATGGATGTGTGGTTGAATTACTGA |
| 5604 | TTCAGTAATTCAACCACACATCCAT | 6868 | ATGGATGTGTGGTTGAATTACTGAA |
| 5605 | TTTCAGTAATTCAACCACACATCCA | 6869 | TGGATGTGTGGTTGAATTACTGAAA |
| 5606 | CTTTCAGTAATTCAACCACACATCC | 6870 | GGATGTGTGGTTGAATTACTGAAAG |
| 5607 | CCTTTCAGTAATTCAACCACACATC | 6871 | GATGTGTGGTTGAATTACTGAAAGG |
| 5608 | ACCTTTCAGTAATTCAACCACACAT | 6872 | ATGTGTGGTTGAATTACTGAAAGGT |
| 5609 | AACCTTTCAGTAATTCAACCACACA | 6873 | TGTGTGGTTGAATTACTGAAAGGTT |
| 5610 | CAACCTTTCAGTAATTCAACCACAC | 6874 | GTGTGGTTGAATTACTGAAAGGTTG |
| 5611 | CCAACCTTTCAGTAATTCAACCACA | 6875 | TGTGGTTGAATTACTGAAAGGTTGG |
| 5612 | TCCAACCTTTCAGTAATTCAACCAC | 6876 | GTGGTTGAATTACTGAAAGGTTGGA |
| 5613 | TTCCAACCTTTCAGTAATTCAACCA | 6877 | TGGTTGAATTACTGAAAGGTTGGAA |
| 5614 | CTTCCAACCTTTCAGTAATTCAACC | 6878 | GGTTGAATTACTGAAAGGTTGGAAG |
| 5615 | TCTTCCAACCTTTCAGTAATTCAAC | 6879 | GTTGAATTACTGAAAGGTTGGAAGA |
| 5616 | CTCTTCCAACCTTTCAGTAATTCAA | 6880 | TTGAATTACTGAAAGGTTGGAAGAG |
| 5617 | ACTCTTCCAACCTTTCAGTAATTCA | 6881 | TGAATTACTGAAAGGTTGGAAGAGT |
| 5618 | CACTCTTCCAACCTTTCAGTAATTC | 6882 | GAATTACTGAAAGGTTGGAAGAGTG |
| 5619 | CCACTCTTCCAACCTTTCAGTAATT | 6883 | AATTACTGAAAGGTTGGAAGAGTGG |
| 5620 | TCCACTCTTCCAACCTTTCAGTAAT | 6884 | ATTACTGAAAGGTTGGAAGAGTGGA |
| 5621 | ATCCACTCTTCCAACCTTTCAGTAA | 6885 | TTACTGAAAGGTTGGAAGAGTGGAT |
| 5622 | CATCCACTCTTCCAACCTTTCAGTA | 6886 | TACTGAAAGGTTGGAAGAGTGGATG |
| 5623 | CCATCCACTCTTCCAACCTTTCAGT | 6887 | ACTGAAAGGTTGGAAGAGTGGATGG |
| 5624 | CCCATCCACTCTTCCAACCTTTCAG | 6888 | CTGAAAGGTTGGAAGAGTGGATGGG |
| 5625 | ACCCATCCACTCTTCCAACCTTTCA | 6889 | TGAAAGGTTGGAAGAGTGGATGGGT |
| 5626 | CACCCATCCACTCTTCCAACCTTTC | 6890 | GAAAGGTTGGAAGAGTGGATGGGTG |
| 5627 | TCACCCATCCACTCTTCCAACCTTT | 6891 | AAAGGTTGGAAGAGTGGATGGGTGA |
| 5628 | TTCACCCATCCACTCTTCCAACCTT | 6892 | AAGGTTGGAAGAGTGGATGGGTGAA |
| 5629 | TTTCACCCATCCACTCTTCCAACCT | 6893 | AGGTTGGAAGAGTGGATGGGTGAAA |
| 5630 | ATTTCACCCATCCACTCTTCCAACC | 6894 | GGTTGGAAGAGTGGATGGGTGAAAT |
| 5631 | AATTTCACCCATCCACTCTTCCAAC | 6895 | GTTGGAAGAGTGGATGGGTGAAATT |
| 5632 | AAATTTCACCCATCCACTCTTCCAA | 6896 | TTGGAAGAGTGGATGGGTGAAATTT |
| 5633 | CAAATTTCACCCATCCACTCTTCCA | 6897 | TGGAAGAGTGGATGGGTGAAATTTG |
| 5634 | CCAAATTTCACCCATCCACTCTTCC | 6898 | GGAAGAGTGGATGGGTGAAATTTGG |
| 5635 | CCCAAATTTCACCCATCCACTCTTC | 6899 | GAAGAGTGGATGGGTGAAATTTGGG |
| 5636 | CCCCAAATTTCACCCATCCACTCTT | 6900 | AAGAGTGGATGGGTGAAATTTGGGG |
| 5637 | ACCCCAAATTTCACCCATCCACTCT | 6901 | AGAGTGGATGGGTGAAATTTGGGGT |
| 5638 | TACCCCAAATTTCACCCATCCACTC | 6902 | GAGTGGATGGGTGAAATTTGGGGTA |
| 5639 | CTACCCCAAATTTCACCCATCCACT | 6903 | AGTGGATGGGTGAAATTTGGGGTAG |
| 5640 | ACTACCCCAAATTTCACCCATCCAC | 6904 | GTGGATGGGTGAAATTTGGGGTAGT |
| 5641 | AACTACCCCAAATTTCACCCATCCA | 6905 | TGGATGGGTGAAATTTGGGGTAGTT |
| 5642 | TAACTACCCCAAATTTCACCCATCC | 6906 | GGATGGGTGAAATTTGGGGTAGTTA |
| 5643 | CTAACTACCCCAAATTTCACCCATC | 6907 | GATGGGTGAAATTTGGGGTAGTTAG |
| 5644 | TCTAACTACCCCAAATTTCACCCAT | 6908 | ATGGGTGAAATTTGGGGTAGTTAGA |
| 5645 | ATCTAACTACCCCAAATTTCACCCA | 6909 | TGGGTGAAATTTGGGGTAGTTAGAT |
| 5646 | CATCTAACTACCCCAAATTTCACCC | 6910 | GGGTGAAATTTGGGGTAGTTAGATG |
| 5647 | CCATCTAACTACCCCAAATTTCACC | 6911 | GGTGAAATTTGGGGTAGTTAGATGG |
| 5648 | CCCATCTAACTACCCCAAATTTCAC | 6912 | GTGAAATTTGGGGTAGTTAGATGGG |
| 5649 | ACCCATCTAACTACCCCAAATTTCA | 6913 | TGAAATTTGGGGTAGTTAGATGGGT |
| 5650 | CACCCATCTAACTACCCCAAATTTC | 6914 | GAAATTTGGGGTAGTTAGATGGGTG |
| 5651 | CCACCCATCTAACTACCCCAAATTT | 6915 | AAATTTGGGGTAGTTAGATGGGTGG |
| 5652 | CCCACCCATCTAACTACCCCAAATT | 6916 | AATTTGGGGTAGTTAGATGGGTGGG |
| 5653 | ACCCACCCATCTAACTACCCCAAAT | 6917 | ATTTGGGGTAGTTAGATGGGTGGGT |
| 5654 | CACCCACCCATCTAACTACCCCAAA | 6918 | TTTGGGGTAGTTAGATGGGTGGGTG |
| 5655 | ACACCCACCCATCTAACTACCCCAA | 6919 | TTGGGGTAGTTAGATGGGTGGGTGT |
| 5656 | CACACCCACCCATCTAACTACCCCA | 6920 | TGGGGTAGTTAGATGGGTGGGTGTG |
| 5657 | ACACACCCACCCATCTAACTACCCC | 6921 | GGGGTAGTTAGATGGGTGGGTGTGT |
| 5658 | CACACACCCACCCATCTAACTACCC | 6922 | GGGTAGTTAGATGGGTGGGTGTGTG |
| 5659 | CCACACACCCACCCATCTAACTACC | 6923 | GGTAGTTAGATGGGTGGGTGTGTGG |
| 5660 | TCCACACACCCACCCATCTAACTAC | 6924 | GTAGTTAGATGGGTGGGTGTGTGGA |
| 5661 | ATCCACACACCCACCCATCTAACTA | 6925 | TAGTTAGATGGGTGGGTGTGTGGAT |
| 5662 | CATCCACACACCCACCCATCTAACT | 6926 | AGTTAGATGGGTGGGTGTGTGGATG |
| 5663 | CCATCCACACACCCACCCATCTAAC | 6927 | GTTAGATGGGTGGGTGTGTGGATGG |
| 5664 | TCCATCCACACACCCACCCATCTAA | 6928 | TTAGATGGGTGGGTGTGTGGATGGA |
| 5665 | ATCCATCCACACACCCACCCATCTA | 6929 | TAGATGGGTGGGTGTGTGGATGGAT |
| 5666 | TATCCATCCACACACCCACCCATCT | 6930 | AGATGGGTGGGTGTGTGGATGGATA |
| 5667 | TTATCCATCCACACACCCACCCATC | 6931 | GATGGGTGGGTGTGTGGATGGATAA |
| 5668 | TTTATCCATCCACACACCCACCCAT | 6932 | ATGGGTGGGTGTGTGGATGGATAAA |
| 5669 | TTTTATCCATCCACACACCCACCCA | 6933 | TGGGTGGGTGTGTGGATGGATAAAA |
| 5670 | CTTTTATCCATCCACACACCCACCC | 6934 | GGGTGGGTGTGTGGATGGATAAAAG |
| 5671 | TCTTTTATCCATCCACACACCCACC | 6935 | GGTGGGTGTGTGGATGGATAAAAGA |
| 5672 | CTCTTTTATCCATCCACACACCCAC | 6936 | GTGGGTGTGTGGATGGATAAAAGAG |
| 5673 | ACTCTTTTATCCATCCACACACCCA | 6937 | TGGGTGTGTGGATGGATAAAAGAGT |
| 5674 | TACTCTTTTATCCATCCACACACCC | 6938 | GGGTGTGTGGATGGATAAAAGAGTA |
| 5675 | CTACTCTTTTATCCATCCACACACC | 6939 | GGTGTGTGGATGGATAAAAGAGTAG |
| 5676 | TCTACTCTTTTATCCATCCACACAC | 6940 | GTGTGTGGATGGATAAAAGAGTAGA |
| 5677 | ATCTACTCTTTTATCCATCCACACA | 6941 | TGTGTGGATGGATAAAAGAGTAGAT |
| 5678 | CATCTACTCTTTTATCCATCCACAC | 6942 | GTGTGGATGGATAAAAGAGTAGATG |
| 5679 | TCATCTACTCTTTTATCCATCCACA | 6943 | TGTGGATGGATAAAAGAGTAGATGA |
| 5680 | TTCATCTACTCTTTTATCCATCCAC | 6944 | GTGGATGGATAAAAGAGTAGATGAA |
| 5681 | ATTCATCTACTCTTTTATCCATCCA | 6945 | TGGATGGATAAAAGAGTAGATGAAT |
| 5682 | CATTCATCTACTCTTTTATCCATCC | 6946 | GGATGGATAAAAGAGTAGATGAATG |
| 5683 | TCATTCATCTACTCTTTTATCCATC | 6947 | GATGGATAAAAGAGTAGATGAATGA |
| 5684 | TTCATTCATCTACTCTTTTATCCAT | 6948 | ATGGATAAAAGAGTAGATGAATGAA |
| 5685 | ATTCATTCATCTACTCTTTTATCCA | 6949 | TGGATAAAAGAGTAGATGAATGAAT |
| 5686 | AATTCATTCATCTACTCTTTTATCC | 6950 | GGATAAAAGAGTAGATGAATGAATT |
| 5687 | TAATTCATTCATCTACTCTTTTATC | 6951 | GATAAAAGAGTAGATGAATGAATTA |
| 5688 | TTAATTCATTCATCTACTCTTTTAT | 6952 | ATAAAAGAGTAGATGAATGAATTAA |
| 5689 | ATTAATTCATTCATCTACTCTTTTA | 6953 | TAAAAGAGTAGATGAATGAATTAAT |
| 5690 | CATTAATTCATTCATCTACTCTTTT | 6954 | AAAAGAGTAGATGAATGAATTAATG |
| 5691 | TCATTAATTCATTCATCTACTCTTT | 6955 | AAAGAGTAGATGAATGAATTAATGA |
| 5692 | TTCATTAATTCATTCATCTACTCTT | 6956 | AAGAGTAGATGAATGAATTAATGAA |
| 5693 | ATTCATTAATTCATTCATCTACTCT | 6957 | AGAGTAGATGAATGAATTAATGAAT |
| 5694 | TATTCATTAATTCATTCATCTACTC | 6958 | GAGTAGATGAATGAATTAATGAATA |
| 5695 | TTATTCATTAATTCATTCATCTACT | 6959 | AGTAGATGAATGAATTAATGAATAA |
| 5696 | TTTATTCATTAATTCATTCATCTAC | 6960 | GTAGATGAATGAATTAATGAATAAA |
| 5697 | GTTTATTCATTAATTCATTCATCTA | 6961 | TAGATGAATGAATTAATGAATAAAC |
| 5698 | TGTTTATTCATTAATTCATTCATCT | 6962 | AGATGAATGAATTAATGAATAAACA |
| 5699 | CTGTTTATTCATTAATTCATTCATC | 6963 | GATGAATGAATTAATGAATAAACAG |
| 5700 | CCTGTTTATTCATTAATTCATTCAT | 6964 | ATGAATGAATTAATGAATAAACAGG |
| 5701 | GCCTGTTTATTCATTAATTCATTCA | 6965 | TGAATGAATTAATGAATAAACAGGC |
| 5702 | TGCCTGTTTATTCATTAATTCATTC | 6966 | GAATGAATTAATGAATAAACAGGCA |
| 5703 | CTGCCTGTTTATTCATTAATTCATT | 6967 | AATGAATTAATGAATAAACAGGCAG |
| 5704 | TCTGCCTGTTTATTCATTAATTCAT | 6968 | ATGAATTAATGAATAAACAGGCAGA |
| 5705 | ATCTGCCTGTTTATTCATTAATTCA | 6969 | TGAATTAATGAATAAACAGGCAGAT |
| 5706 | CATCTGCCTGTTTATTCATTAATTC | 6970 | GAATTAATGAATAAACAGGCAGATG |
| 5707 | CCATCTGCCTGTTTATTCATTAATT | 6971 | AATTAATGAATAAACAGGCAGATGG |
| 5708 | TCCATCTGCCTGTTTATTCATTAAT | 6972 | ATTAATGAATAAACAGGCAGATGGA |
| 5709 | ATCCATCTGCCTGTTTATTCATTAA | 6973 | TTAATGAATAAACAGGCAGATGGAT |
| 5710 | CATCCATCTGCCTGTTTATTCATTA | 6974 | TAATGAATAAACAGGCAGATGGATG |
| 5711 | TCATCCATCTGCCTGTTTATTCATT | 6975 | AATGAATAAACAGGCAGATGGATGA |
| 5712 | ATCATCCATCTGCCTGTTTATTCAT | 6976 | ATGAATAAACAGGCAGATGGATGAT |
| 5713 | CATCATCCATCTGCCTGTTTATTCA | 6977 | TGAATAAACAGGCAGATGGATGATG |
| 5714 | ACATCATCCATCTGCCTGTTTATTC | 6978 | GAATAAACAGGCAGATGGATGATGT |
| 5715 | TACATCATCCATCTGCCTGTTTATT | 6979 | AATAAACAGGCAGATGGATGATGTA |
| 5716 | TTACATCATCCATCTGCCTGTTTAT | 6980 | ATAAACAGGCAGATGGATGATGTAA |
| 5717 | CTTACATCATCCATCTGCCTGTTTA | 6981 | TAAACAGGCAGATGGATGATGTAAG |
| 5718 | GCTTACATCATCCATCTGCCTGTTT | 6982 | AAACAGGCAGATGGATGATGTAAGC |
| 5719 | AGCTTACATCATCCATCTGCCTGTT | 6983 | AACAGGCAGATGGATGATGTAAGCT |
| 5720 | CAGCTTACATCATCCATCTGCCTGT | 6984 | ACAGGCAGATGGATGATGTAAGCTG |
| 5721 | GCAGCTTACATCATCCATCTGCCTG | 6985 | CAGGCAGATGGATGATGTAAGCTGC |
| 5722 | GGCAGCTTACATCATCCATCTGCCT | 6986 | AGGCAGATGGATGATGTAAGCTGCC |
| 5723 | GGGCAGCTTACATCATCCATCTGCC | 6987 | GGCAGATGGATGATGTAAGCTGCCC |
| 5724 | GGGGCAGCTTACATCATCCATCTGC | 6988 | GCAGATGGATGATGTAAGCTGCCCC |
| 5725 | TGGGGCAGCTTACATCATCCATCTG | 6989 | CAGATGGATGATGTAAGCTGCCCCA |
| 5726 | CTGGGGCAGCTTACATCATCCATCT | 6990 | AGATGGATGATGTAAGCTGCCCCAG |
| 5727 | TCTGGGGCAGCTTACATCATCCATC | 6991 | GATGGATGATGTAAGCTGCCCCAGA |
| 5728 | GTCTGGGGCAGCTTACATCATCCAT | 6992 | ATGGATGATGTAAGCTGCCCCAGAC |
| 5729 | GGTCTGGGGCAGCTTACATCATCCA | 6993 | TGGATGATGTAAGCTGCCCCAGACC |
| 5730 | GGGTCTGGGGCAGCTTACATCATCC | 6994 | GGATGATGTAAGCTGCCCCAGACCC |
| 5731 | AGGGTCTGGGGCAGCTTACATCATC | 6995 | GATGATGTAAGCTGCCCCAGACCCT |
| 5732 | CAGGGTCTGGGGCAGCTTACATCAT | 6996 | ATGATGTAAGCTGCCCCAGACCCTG |
| 5733 | CCAGGGTCTGGGGCAGCTTACATCA | 6997 | TGATGTAAGCTGCCCCAGACCCTGG |
| 5734 | CCCAGGGTCTGGGGCAGCTTACATC | 6998 | GATGTAAGCTGCCCCAGACCCTGGG |
| 5735 | TCCCAGGGTCTGGGGCAGCTTACAT | 6999 | ATGTAAGCTGCCCCAGACCCTGGGA |
| 5736 | GTCCCAGGGTCTGGGGCAGCTTACA | 7000 | TGTAAGCTGCCCCAGACCCTGGGAC |
| 5737 | GGTCCCAGGGTCTGGGGCAGCTTAC | 7001 | GTAAGCTGCCCCAGACCCTGGGACC |
| 5738 | AGGTCCCAGGGTCTGGGGCAGCTTA | 7002 | TAAGCTGCCCCAGACCCTGGGACCT |
| 5739 | GAGGTCCCAGGGTCTGGGGCAGCTT | 7003 | AAGCTGCCCCAGACCCTGGGACCTC |
| 5740 | AGAGGTCCCAGGGTCTGGGGCAGCT | 7004 | AGCTGCCCCAGACCCTGGGACCTCT |
| 5741 | CAGAGGTCCCAGGGTCTGGGGCAGC | 7005 | GCTGCCCCAGACCCTGGGACCTCTG |
| 5742 | TCAGAGGTCCCAGGGTCTGGGGCAG | 7006 | CTGCCCCAGACCCTGGGACCTCTGA |
| 5743 | GTCAGAGGTCCCAGGGTCTGGGGCA | 7007 | TGCCCCAGACCCTGGGACCTCTGAC |
| 5744 | GGTCAGAGGTCCCAGGGTCTGGGGC | 7008 | GCCCCAGACCCTGGGACCTCTGACC |
| 5745 | GGGTCAGAGGTCCCAGGGTCTGGGG | 7009 | CCCCAGACCCTGGGACCTCTGACCC |
| 5746 | GGGGTCAGAGGTCCCAGGGTCTGGG | 7010 | CCCAGACCCTGGGACCTCTGACCCC |
| 5747 | GGGGGTCAGAGGTCCCAGGGTCTGG | 7011 | CCAGACCCTGGGACCTCTGACCCCC |
| 5748 | CGGGGGTCAGAGGTCCCAGGGTCTG | 7012 | CAGACCCTGGGACCTCTGACCCCCG |
| 5749 | CCGGGGGTCAGAGGTCCCAGGGTCT | 7013 | AGACCCTGGGACCTCTGACCCCCGG |
| 5750 | GCCGGGGGTCAGAGGTCCCAGGGTC | 7014 | GACCCTGGGACCTCTGACCCCCGGC |
| 5751 | CGCCGGGGGTCAGAGGTCCCAGGGT | 7015 | ACCCTGGGACCTCTGACCCCCGGCG |
| 5752 | TCGCCGGGGGTCAGAGGTCCCAGGG | 7016 | CCCTGGGACCTCTGACCCCCGGCGA |
| 5753 | GTCGCCGGGGGTCAGAGGTCCCAGG | 7017 | CCTGGGACCTCTGACCCCCGGCGAC |
| 5754 | GGTCGCCGGGGGTCAGAGGTCCCAG | 7018 | CTGGGACCTCTGACCCCCGGCGACC |
| 5755 | GGGTCGCCGGGGGTCAGAGGTCCCA | 7019 | TGGGACCTCTGACCCCCGGCGACCC |
| 5756 | GGGGTCGCCGGGGGTCAGAGGTCCC | 7020 | GGGACCTCTGACCCCCGGCGACCCC |
| 5757 | AGGGGTCGCCGGGGGTCAGAGGTCC | 7021 | GGACCTCTGACCCCCGGCGACCCCT |
| 5758 | AAGGGGTCGCCGGGGGTCAGAGGTC | 7022 | GACCTCTGACCCCCGGCGACCCCTT |
| 5759 | CAAGGGGTCGCCGGGGGTCAGAGGT | 7023 | ACCTCTGACCCCCGGCGACCCCTTG |
| 5760 | GCAAGGGGTCGCCGGGGGTCAGAGG | 7024 | CCTCTGACCCCCGGCGACCCCTTGC |
| 5761 | TGCAAGGGGTCGCCGGGGGTCAGAG | 7025 | CTCTGACCCCCGGCGACCCCTTGCA |
| 5762 | GTGCAAGGGGTCGCCGGGGGTCAGA | 7026 | TCTGACCCCCGGCGACCCCTTGCAC |
| 5763 | AGTGCAAGGGGTCGCCGGGGGTCAG | 7027 | CTGACCCCCGGCGACCCCTTGCACT |
| 5764 | GAGTGCAAGGGGTCGCCGGGGGTCA | 7028 | TGACCCCCGGCGACCCCTTGCACTC |
| 5765 | AGAGTGCAAGGGGTCGCCGGGGGTC | 7029 | GACCCCCGGCGACCCCTTGCACTCT |
| 5766 | GAGAGTGCAAGGGGTCGCCGGGGGT | 7030 | ACCCCCGGCGACCCCTTGCACTCTC |
| 5767 | GGAGAGTGCAAGGGGTCGCCGGGGG | 7031 | CCCCCGGCGACCCCTTGCACTCTCC |
| 5768 | TGGAGAGTGCAAGGGGTCGCCGGGG | 7032 | CCCCGGCGACCCCTTGCACTCTCCA |
| 5769 | ATGGAGAGTGCAAGGGGTCGCCGGG | 7033 | CCCGGCGACCCCTTGCACTCTCCAT |
| 5770 | CATGGAGAGTGCAAGGGGTCGCCGG | 7034 | CCGGCGACCCCTTGCACTCTCCATG |
| 5771 | TCATGGAGAGTGCAAGGGGTCGCCG | 7035 | CGGCGACCCCTTGCACTCTCCATGA |
| 5772 | GTCATGGAGAGTGCAAGGGGTCGCC | 7036 | GGCGACCCCTTGCACTCTCCATGAC |
| 5773 | TGTCATGGAGAGTGCAAGGGGTCGC | 7037 | GCGACCCCTTGCACTCTCCATGACA |
| 5774 | GTGTCATGGAGAGTGCAAGGGGTCG | 7038 | CGACCCCTTGCACTCTCCATGACAC |
| 5775 | AGTGTCATGGAGAGTGCAAGGGGTC | 7039 | GACCCCTTGCACTCTCCATGACACT |
| 5776 | AAGTGTCATGGAGAGTGCAAGGGGT | 7040 | ACCCCTTGCACTCTCCATGACACTT |
| 5777 | AAAGTGTCATGGAGAGTGCAAGGGG | 7041 | CCCCTTGCACTCTCCATGACACTTT |
| 5778 | GAAAGTGTCATGGAGAGTGCAAGGG | 7042 | CCCTTGCACTCTCCATGACACTTTC |
| 5779 | AGAAAGTGTCATGGAGAGTGCAAGG | 7043 | CCTTGCACTCTCCATGACACTTTCT |
| 5780 | GAGAAAGTGTCATGGAGAGTGCAAG | 7044 | CTTGCACTCTCCATGACACTTTCTC |
| 5781 | AGAGAAAGTGTCATGGAGAGTGCAA | 7045 | TTGCACTCTCCATGACACTTTCTCT |
| 5782 | GAGAGAAAGTGTCATGGAGAGTGCA | 7046 | TGCACTCTCCATGACACTTTCTCTC |
| 5783 | GGAGAGAAAGTGTCATGGAGAGTGC | 7047 | GCACTCTCCATGACACTTTCTCTCC |
| 5784 | GGGAGAGAAAGTGTCATGGAGAGTG | 7048 | CACTCTCCATGACACTTTCTCTCCC |
| 5785 | TGGGAGAGAAAGTGTCATGGAGAGT | 7049 | ACTCTCCATGACACTTTCTCTCCCA |
| 5786 | ATGGGAGAGAAAGTGTCATGGAGAG | 7050 | CTCTCCATGACACTTTCTCTCCCAT |
| 5787 | CATGGGAGAGAAAGTGTCATGGAGA | 7051 | TCTCCATGACACTTTCTCTCCCATG |
| 5788 | CCATGGGAGAGAAAGTGTCATGGAG | 7052 | CTCCATGACACTTTCTCTCCCATGG |
| 5789 | ACCATGGGAGAGAAAGTGTCATGGA | 7053 | TCCATGACACTTTCTCTCCCATGGT |
| 5790 | CACCATGGGAGAGAAAGTGTCATGG | 7054 | CCATGACACTTTCTCTCCCATGGTG |
| 5791 | CCACCATGGGAGAGAAAGTGTCATG | 7055 | CATGACACTTTCTCTCCCATGGTGG |
| 5792 | GCCACCATGGGAGAGAAAGTGTCAT | 7056 | ATGACACTTTCTCTCCCATGGTGGC |
| 5793 | TGCCACCATGGGAGAGAAAGTGTCA | 7057 | TGACACTTTCTCTCCCATGGTGGCA |
| 5794 | CTGCCACCATGGGAGAGAAAGTGTC | 7058 | GACACTTTCTCTCCCATGGTGGCAG |

*At least one (i.e., one or more) nucleoside linkage of the oligonucleotide sequence is independently selected from a phosphorothioate linkage, an alkyl phosphate linkage, a phosphorodithioate linkage, a phosphotriester linkage, an alkylphosphonate linkage, a 3- methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

### UNC13A Transcript

In various embodiments, an UNC13A mRNA transcript comprises the sequence provided as SEQ ID NO: 9587.

(SEQ ID NO: 9587 (SOURCE NCBI Reference Sequence NM_001080421.3))
GCCCCCGGTGCTGAACCAAGATGGCCGGTGGCGGCCGGGCCCCGGCGTGAGCCAAGCGCGGGCTGCAGCC
GGGAGATGCCCCAGCCCAGCGGCCGCTGAGCCCGACCCGACAGAGCCGGCCCGGCCGCCTCCGGCCCACC
TGCGAGCTCGGAGACATGTCTCTGCTTTGCGTTGGAGTCAAAAAAGCCAAGTTTGATGGTGCCCAAGAGA
AATTCAACACGTACGTGACCCTGAAAGTGCAGAATGTCAAGAGCACGACCATCGCGGTGCGGGGCAGCCA
GCCCAGCTGGGAGCAGGATTTCATGTTCGAGATTAACCGTCTGGATTTGGGACTGACGGTGGAGGTGTGG
AATAAGGGTCTCATCTGGGACACAATGGTGGGCACTGTGTGGATCCCACTGAGGACCATCCGCCAGTCCA
ATGAGGAGGGCCCTGGAGAGTGGCTGACGCTGGACTCCCAGGTCATCATGGCAGACAGTGAGATCTGTGG
CACCAAGGACCCCACCTTCCACCGCATCCTCCTGGACACGCGCTTTGAGCTACCCTTAGACATTCCTGAA
GAGGAGGCTCGCTACTGGGCCAAGAAGCTGGAGCAGCTCAATGCTATGCGGGACCAGGATGAATATTCGT
TCCAAGATGAGCAAGACAAGCCTCTGCCTGTCCCCAGCAACCAGTGCTGCAACTGGAATTATTTTGGCTG
GGGTGAGCAGCACAACGATGACCCCGACAGTGCAGTGGATGATCGTGACAGTGACTACCGCAGTGAAACG
AGCAACAGCATCCCGCCGCCCTATTATACTACGTCACAACCCAACGCCTCAGTCCACCAATATTCTGTTC
GCCCACCACCCCTGGGCTCCCGGGAGTCCTACAGTGACTCCATGCACAGTTACGAGGAGTTCTCTGAGCC
ACAAGCCCTCAGCCCCACGGGTAGCAGCCGCTATGCCTCTTCCGGGGAGCTGAGCCAGGGAAGCTCTCAG
CTGAGCGAGGACTTCGACCCTGACGAGCACAGCCTGCAGGGCTCCGACATGGAGGATGAGCGGGACCGGG
ACTCCTACCACTCCTGCCACAGCTCGGTCAGCTACCACAAAGACTCGCCTCGCTGGGACCAGGATGAGGA
AGAGCTGGAGGAGGACCTGGAGGACTTCCTGGAGGAGGAGGAGCTGCCTGAAGATGAGGAGGAGCTGGAG
GAGGAGGAGGAGGAGGTGCCTGACGATTTGGGCAGCTATGCCCAGCGTGAAGACGTAGCTGTGGCTGAGC
CCAAAGACTTCAAACGCATCAGCCTCCCGCCAGCTGCCCCAGGGAAGGAGGACAAGGCCCCAGTGGCACC
CACCGAGGCCCCCGACATGGCCAAGGTGGCCCCCAAGCCAGCCACGCCCGACAAGGTGCCTGCAGCTGAG
CAGATCCCTGAGGCTGAGCCACCCAAGGACGAGGAGAGTTTCAGGCCGAGAGAGGATGAGGAAGGCCAGG
AGGGGCAGGACTCCATGTCCAGGGCCAAGGCCAACTGGCTGCGTGCCTTCAACAAGGTGCGGATGCAGCT
GCAGGAGGCCCGGGGAGAAGGAGAGATGTCTAAATCCCTATGGTTCAAAGGCGGCCCAGGGGGCGGTCTC
ATCATCATCGACAGCATGCCAGACATCCGCAAGAGGAAACCTATCCCACTCGTGAGCGACTTGGCCATGT
CCCTGGTCCAGTCCAGGAAAGCGGGCATCACCTCGGCCTTGGCCTCCAGCACGTTGAACAACGAGGAGCT
GAAAAACCACGTTTACAAGAAGACCCTGCAAGCCTTAATCTACCCCATCTCGTGCACGACGCCACACAAC
TTCGAAGTGTGGACGGCCACCACGCCCACCTACTGCTACGAGTGCGAGGGGCTGCTGTGGGGCATCGCGA
GGCAGGGCATGCGCTGCACCGAGTGCGGTGTCAAGTGCCACGAGAAGTGCCAGGACCTGCTCAACGCCGA
CTGCCTGCAGCGGGCTGCGGAGAAGAGCTCCAAGCACGGGGGGAGGACCGGACACAGAACATCATCATG
GTGCTCAAGGACCGCATGAAGATCCGGGAGCGCAACAAGCCCGAGATCTTCGAGCTCATCCAGGAGATCT
TCGCGGTGACCAAGACGGCGCACACGCAGCAGATGAAGGCGGTCAAGCAGAGCGTGCTGGACGGCACGTC
CAAGTGGTCCGCCAAGATCAGCATCACCGTGGTCTGCGCCCAGGGCTTGCAGGCAAAGGACAAGACAGGA
TCCAGTGACCCCTATGTCACCGTCCAGGTCGGGAAGACCAAGAAACGGACAAAAACCATCTATGGGAACC
TCAACCCGGTGTGGGAGGAGAATTTCCACTTTGAATGTCACAATTCCTCCGACCGCATCAAGGTGCGCGT
CTGGGACGAGGATGACGACATCAAATCCCGCGTGAAACAGAGGTTCAAGAGGGAATCTGACGATTTCCTG
GGGCAGACGATCATTGAGGTGCGGACGCTCAGCGGCGAGATGGACGTGTGGTACAACCTGGACAAGCGAA
CTGACAAATCTGCCGTGTCGGGTGCCATCCGGCTCCACATCAGTGTGGAGATCAAAGGCGAGGAGAAGGT
GGCCCCGTACCATGTCCAGTACACCTGTCTGCATGAGAACCTGTTCCACTTCGTGACCGACGTGCAGAAC
AATGGGGTCGTGAAGATCCCAGATGCCAAGGGTGACGATGCCTGGAAGGTTTACTACGATGAGACAGCCC
AGGAGATTGTGGACGAGTTTGCCATGCGCTACGGCGTCGAGTCCATCTACCAAGCCATGACCCACTTTGC
CTGCCTCTCCTCCAAGTATATGTGCCCAGGGGTGCCTGCCGTCATGAGCACCCTGCTCGCCAACATCAAT
GCCTACTACGCACACACCACCGCCTCCACCAACGTGTCTGCCTCCGACCGCTTCGCCGCCTCCAACTTTG
GGAAAGAGCGCTTCGTGAAACTCCTGGACCAGCTGCATAACTCCCTGCGGATTGACCTCTCCATGTACCG
GAATAACTTCCCAGCCAGCAGCCCGGAGAGACTCCAGGACCTCAAATCCACTGTGGACCTTCTCACCAGC
ATCACCTTCTTTCGGATGAAGGTACAAGAACTCCAGAGCCCGCCCCGAGCCAGCCAGGTGGTAAAGGACT
GTGTGAAAGCCTGCCTTAATTCTACCTACGAGTACATCTTCAATAACTGCCATGAACTGTACAGCCGGGA
GTACCAGACAGACCCGGCCAAGAAGGGGGAAGTTCTCCCAGAGGAACAGGGGCCCAGCATCAAGAACCTC
GACTTCTGGTCCAAGCTGATTACCCTCATAGTGTCCATCATTGAGGAAGACAAGAATTCCTACACTCCCT
GCCTCAACCAGTTTCCCCAGGAGCTGAATGTGGGTAAAATCAGCGCTGAAGTGATGTGGAATCTGTTTGC
CCAAGACATGAAGTACGCCATGGAGGAGCACGACAAGCATCGTCTATGCAAGAGTGCCGACTACATGAAC
CTCCACTTCAAGGTGAAATGGCTCTACAATGAGTATGTGACGGAACTTCCCGCCTTCAAGGACCGCGTGC
CTGAGTACCCTGCATGGTTTGAACCCTTCGTCATCCAGTGGCTGGATGAGAATGAGGAGGTGTCCCGGGA
TTTCCTGCACGGTGCCCTGGAGCGAGACAAGAAGGATGGGTTCCAGCAGACCTCAGAGCATGCCCTATTC
TCCTGCTCCGTGGTGGATGTTTTCTCCCAACTCAACCAGAGCTTTGAAATCATCAAGAAACTCGAGTGTC
CCGACCCTCAGATCGTGGGGCACTACATGAGGCGCTTTGCCAAGACCATCAGTAATGTGCTCCTCCAGTA
TGCAGACATCATCTCCAAGGACTTTGCCTCCTACTGCTCCAAGGAGAAGGAGAAAGTGCCCTGCATTCTC
ATGAATAACACTCAACAGCTACGAGTTCAGCTGGAGAAGATGTTCGAAGCCATGGGAGGAAAGGAGCTGG
ATGCTGAAGCCAGTGACATCCTGAAGGAGCTTCAGGTGAAACTCAATAACGTCTTGGATGAGCTCAGCCG
GGTGTTTGCTACCAGCTTCCAGCCGCACATTGAAGAGTGTGTCAAACAGATGGGTGACATCCTTAGCCAG
GTTAAGGGCACAGGCAATGTGCCAGCCAGTGCCTGCAGCAGCGTGGCCCAGGACGCGGACAATGTGTTGC
AGCCCATCATGGACCTGCTGGACAGCAACCTGACCCTCTTTGCCAAAATCTGTGAGAAGACTGTGCTGAA
GCGAGTGCTGAAGGAGCTGTGGAAGCTGGTTATGAACACCATGGAGAAAACCATCGTCCTGCCGCCCCTC
ACTGACCAGACGATGATCGGGAACCTCTTGAGAAAACATGGCAAGGGATTAGAAAAGGGCAGGGTGAAAT
TGCCAAGCCACTCAGACGGAACCCAGATGATCTTCAATGCAGCCAAGGAGCTGGGTCAGCTGTCCAAACT
CAAGGATCACATGGTACGAGAAGAAGCCAAGAGCTTGACCCCAAAGCAGTGCGCGGTTGTTGAGTTGGCC
CTGGACACCATCAAGCAATATTTCCACGCGGGTGGCGTGGGCCTCAAGAAGACCTTCCTGGAGAAGAGCC
CGGACCTGCAATCCTTGCGCTATGCCCTGTCGCTCTACACGCAGGCCACCGACCTGCTAATCAAGACCTT
TGTACAGACGCAATCGGCCCAGGGCTTGGGTGTAGAAGACCCTGTGGGTGAAGTCTCTGTCCATGTTGAG
CTGTTCACTCATCCAGGAACTGGGGAACACAAGGTCACAGTGAAAGTGGTGGCTGCCAATGACCTCAAGT
GGCAGACTTCTGGCATCTTCCGGCCGTTCATCGAGGTCAACATCATTGGGCCCCAGCTCAGCGACAAGAA
ACGCAAGTTTGCGACCAAATCCAAGAACAATAGCTGGGCTCCCAAGTACAATGAGAGCTTCCAGTTCACG
CTGAGCGCCGACGCGGGTCCCGAGTGCTATGAGCTGCAGGTGTGCGTCAAGGACTACTGCTTCGCGCGCG
AGGACCGCACGGTGGGGCTGGCCGTGCTGCAGCTGCGTGAGCTGGCCCAGCGCGGGAGCGCCGCCTGCTG
GCTGCCGCTCGGCCGCCGCATCCACATGGACGACACGGGCCTCACGGTGCTGCGAATCCTCTCGCAGCGC
AGCAACGACGAGGTGGCCAAGGAGTTCGTGAAGCTCAAGTCGGACACGCGCTCCGCCGAGGAGGGCGGTG
CCGCGCCTGCGCCTTAGCGCGGGCGGTCGGCCGAGCGGCACTGCGCCTGCGCGGAGGGCGCTGGGCGGGG
AGGGACGGGGCTTGCGCCTTGGTGGGACCTCCCCAGGGGCGGGGCTCGGGGGGCTCCACGCCAAGGGTGG
GCTGCGCCTACGCCCTTGACTCAGCTTTCCCTTTTGGGGAATTAGGAATGGAGGATGCCCCGCCCTCTCG
GGAGGCCACGCCCAAGGGCGCGACGAAGGAAGGAGCCACATCCCCAACTTGAGGCCACGCCCCCAGCACC
TAGGGGGCATTTTGAGCTGGGATGGGGGAAACCTCGTCCCTATGGAGGAGGCCACATCCCGGGGCTCTGG
TACCGGGAGGCACCACCTCATGTCCCCTGGAAAAGCCATAAGATGGGACCCAGACCCCTGGGACCCCAGA
CCAATTGCCAAGTATGGAAATCTCAGCTCCCTCGAGGGGGGGCCCTGGGCAAGGGGTAGGGCTCTCTGGA
GCGCCCCTCTAGGTGGCCTGGGGACTGGAGGGACCAGGATGCTGGTTGGAGGGCCCCGGAATACCGGAGT
CCCTTTAGATATTTGTGCAAAAAATAAATGGGGGGAGGGGGGAGGATGGGATTTCAAAAGCACATGCGCC
CTTGGGCGCCCAAACCCTGGGGGCCGAGGGGACGGCTCTGGTTCCCCACGCTGCCCCTACTTCCCTTTGG
GAGTTTGCCTCTCCCTCTCCCCCAACAAACCCAGTCCTCATATCATAGAGTTCAACACACCCATTTGACA
GATGGCAAAACTGAGGCTTAAAGAGCTGCTTGAGACTTGGCCAAGGTTCCAGGTGCCATACCCTCTGTGC
CCCTCCCTTAGGCCTGTGTGCCCCATGGAAGGGTGGGCTGAGATCGGGATGACCTGACACAGCTCCCTAT
TGCTGCTAATTCCCCCTCGGCCTCCTCCAAGGGGTGGGAATTCCAGGCCAAGACCCCTACTTCGCCTTTC
CTTCTCCGGCTGCCAAGCAGGACCTTTGCCCTCAGCCCTTTCTCCTGGGATCTCCATGGGGGATGCCATG
AGGGCCTCCCACCACAAAAGAGAATTTGGGATCCCCTGGTCCCAGGTTTCTCCATCCCTTCTTCCTTTTC
CAGAATTTTCCAAATAGGAAAGAACAGAAGGAGACCAGAAACTCTAGGGGGGAGAAAGAGAATGAGAGAA
AGAGAATGAGAGAGAGAGAAACACAAACACAGTGACACAGTGAGAGCTTAGTCTCCAAGAGCCTATTCAT
TGATTCAAACACCCAAGCCACAGGATACCTCAGATGGCCCTCTTGCCAGCTGGAAGCTCTTTCTCCAATG
AGCAAAGTTACAGTGACCTGGCTGGAGTTACCTGGTGCACATAGGACCTTAGGGGAAAGTTCAGCGTGGA
CTACACTTGCTCTGGGATCTGCTTTTCCACATGTGTGTATGGCACGCCTTTTTCTGCTGGATTGGGAAGG
ACAAGATTTTGCTGTGCTAGGGAGAAATGAAAACGGGGTGAGCTGAGTAGCTGGGTTTCTGGAGGATAGA
ACATCAGATGGGGAGGCTTTCCGAGGTGAAGAATGAGAGGGAACCACTTACTAGAGAGAAAAGAGCTCCA
GGCCTGGGGAACAGCACGTGCGAAGGCCAGGAGAGAAGAACTGTTGAAACAACGAGAAGGGTGGCACGGC
TGGAGCTGAGCCAGCAAGGGGGATCGTGAGGAGCCTTGGGGTTGGGGAGATCTGCAGAAGCATCAGACCA
GGCAGGGCCTCGTACGCAGTCCTGAGGAGTTTTACTTTTATTCTAAGACAGTTGGGGAGCTCCAGGAGCT
GTTTTAAGTTGGGGAGAGACTGGATTCCAGCCTGCAAAAGCTGTTTTGTGAAGACTAAAACCAGTGAGGA
GAGGTGGAGGTGCTTTGGGGACACTGAAATGGATTCTTGGAAAGATTCTGAAGGCTGTGTTGAAAAGACA
CCTATAGCTGTGGGGACATGACTATAATCCCAGCATTTGGGGAGACCGAGGCTGGCAGATCACTTAAGGT
CAGGAGTTTGAGACCAGCCTGGCCAACATGGCGAAACCCCATCTCTGCTAAAAATACAAAAATTAGCTGG
GTGCAGTGGTGCATGCCTGTAGTCCCAGCTACTCAGGAGACTGAGGCGGGAGAATTGTTTGAACCCTGGA
GGCAGAGGTTGTAGTGAGTCGTGATCACACAACTGCACTCCAGCCTGGGCAACAGAACAATACTCCATTC
CCTCCCCTCTACCCCACCAAAAAAAAAAAAAAATCCTGCCCTTAGATGAGCTCTAGGGCTGCTGAGTACA
GTTGTCCCAGTTGCACAGTGCCCAAGGGTTTGGCATTGCTAAGAAGGCCACGTGCAAATCCTAGATATTG
AGTGTTGTATGTTTGTGACGTTGGTTTCCCGACATGTGAATGGCCCAAGTGTCTGGAAGAAGTGGCGCCA
CTTTCTAATTTGCTTGGAGATGTTGCATGTCCCTTAAATTCAGACAGGTGCAGGTAACTGGAGGTTCTGA
ACCAAAGGTTAAAATGCAAATTCTCATACAGGGTTGGGAAGTTGTAGCCAGGGATAAGCTTATGTGACTG
TTATATGGACTGAGGAGCAGATGTGAATTTCGAACCATGACATGGCTGAGGGTAGGGGTCGGGTGGATGG
ATGATTCAGGGTTGTAACCCATAGAGCCCAAAGGGGAAGTGATCTGTGACCTGGGGTGAGGGTGATCTGG
AAGATTTTTGGATGGCTGGAAAGAAATGGGGAAGTCGAGCTGCCTGAGAGAGCCAAGTTATTTCCCAAAA
GATTCCTTAGGAGTCTTTCTGTTCAAGACCTCCGTGTGTGTGTGTGTGTGTTTAGGGTTCCCCAGCAATG
GCCCAGGCATGTGAAGGAAACAAGCTTCTTCAGGGAATATTTGTTGAATGAGTTTTCCTGACTCCCAGGC
TAGAACTGTTTTTGCAATTTCCACCCTCTTTTCTTTCCCCCAGAGAACTCCTATTCGTCCTTCAAAACCC
ATCACGGAAACCCCTCTTGGAGAAAACCCTCCTTCCTTCCCCTCAGGACTTTCCCAGCCACCGTCTCTCC
TCCAGTCCAGCCTGATGCCATGGGACTGGGGGTTTCTCTGTCCAGCTCTGTTTCTCCCAGACTGGGGTCT
GAGGACTCTCAGGACCCCCAACTTTACCTAGCACAGGCTGGGCACAAGTGGGTGACAGGGAGTCTACGCC
TAGTGGAATTATGTATTGGGGCAGGGTCAGTGTGAGAATACACATCCGCATGCATGTCTGTCCATGTCTG
TCCGTACCAACCTTCCCCTTCCACACGGACCTGGGCACATAGGAGGTGTCTGAGCCTGACACATGGGACA
GAGAGTGGACATGGCTGAGACACGGACAGAGAAAAGACAAGGAGTCCAGGGGGCTGAAAGCCTTTTGAAA
TCAGGAAGTTCCTGTATTGGCAGAACAAAGCCCAGAGAGGAGCAGGGCTTTCCTCAACGCCACCCAGCAA
GTGGACACAGAGCCCGGCCTTGGATGACACCTCCAGGGTTCTGAACCCTGGACCTCGCTTTATGCAAGGA
GCTGGCCCCACATTTCCATGAATCGGGGAAACAGCACAAGAAGGTTGGCCTGTGGCAGGGCAAGGGTTAA
AGGGGTGACATTGAGGGATGCCTCAGAGTCAAAGTCCCCTGACCAAGAGGAATAGAGTAGAAAACACAGA
GACAGAGGGTGAGATCACGCCCCGATGAGGACGGAGAGAGACAGAGATGGAGAGAGACATAGAGGTGGAA
ATATACAGAGAAAGATAAATGCAGAGACCAAGGCAGGGAGTGTCGGGGGAAGTAAAGAGGGTGTCCTGAA
GAAAGAAGGATCTGTTCACTCTTACCAGTCTGTCCTCGAATGATTTGCATAAAATGAGGAGGTGCCTGTC
CACACCCCCAATTCCTCTCTCAGGCCCCAGAGCCTGAGACCTCACCATGCCCCCATCAGAGATGCAAAAA
ACTAAACACCCAACTAGAAATCCTTGGGACCTCTCTCGGCTGGGATCTCAGAGCCTTTCTGTCCCCTACC
CCTACCCCATGTGCTGTCGATTTTGCAGATGGGGACAACCTGGGGCCTCCCGGAACTCTGCCACCCTGGG
GAAGTTGGGGGAGGGCCTTAGTCCCGGATCACAACCCCGTCTGCTCCCCAGAATCCTTTCCTAAGAATCG
TTGAGGACCAAAGTTGTCTTTGCTGACACGTGTTGCTTTTCTCTTTGCCTTTTATTGTTTCAGAGAAAAA
TCAAGTTGACTGTGTCAAGTAACACCCCACCCCTTACCCCCGTCCAGCCATAGTGGCTCTCTGGAGACAC
AGGTCACAGGCGGAGGGTCCCCTGATCATCCCCAACCACACAGCCAGGGGGACTTGACCCCTGTCCACCC
CTGTCTCGTGCTCCCTCAGACCCCCACAAACCGGCCAAGCAGTCCGGGGAGGCTTCCCCTCCACACAACT
CTTAGCATGTGATTGCAGATGTGAAATCAAAACGTTGTTTGTTTTTTGTTTTGTTTTGATTCTACCCCGT
CGGTCCAGTGTCTGCACAGACGCCTTCATTTCTCTGTAAATATGTGACTTGGAACAAATGTTTAACACAA
ACGAGAAGTGGTCATGAATGCATGGTGTTGAGATGTTTTGCACTATTCTGACTTTTTGGTCTCTGTAAAA
ATATTTTATTAACAGCAGACATTAAAAAAAGAAAAACCACACACA.

In various embodiments, an UNC13A mRNA transcript comprises the sequence provided as SEQ ID NO: 9588.

(SEQ ID NO: 9588 (SOURCE NCBI Reference Sequence NM_001387021.1)
GCCCCCGGTGCTGAACCAAGATGGCCGGTGGCGGCCGGGCCCCGGCGTGAGCCAAGCGCGGGCTGCAGCC
GGGAGATGCCCCAGCCCAGCGGCCGCTGAGCCCGACCCGACAGAGCCGGCCCGGCCGCCTCCGGCCCACC
TGCGAGCTCGGAGACATGTCTCTGCTTTGCGTTGGAGTCAAAAAAGCCAAGTTTGATGGTGCCCAAGAGA
AATTCAACACGTACGTGACCCTGAAAGTGCAGAATGTCAAGAGCACGACCATCGCGGTGCGGGGCAGCCA
GCCCAGCTGGGAGCAGGATTTCATGTTCGAGATTAACCGTCTGGATTTGGGACTGACGGTGGAGGTGTGG
AATAAGGGTCTCATCTGGGACACAATGGTGGGCACTGTGTGGATCCCACTGAGGACCATCCGCCAGTCCA
ATGAGGAGGGCCCTGGAGAGTGGCTGACGCTGGACTCCCAGGTCATCATGGCAGACAGTGAGATCTGTGG
CACCAAGGACCCCACCTTCCACCGCATCCTCCTGGACACGCGCTTTGAGCTACCCTTAGACATTCCTGAA
GAGGAGGCTCGCTACTGGGCCAAGAAGCTGGAGCAGCTCAATGCTATGCGGGACCAGGATGAATATTCGT
TCCAAGATGAGCAAGACAAGCCTCTGCCTGTCCCCAGCAACCAGTGCTGCAACTGGAATTATTTTGGCTG
GGGTGAGCAGCACAACGATGACCCCGACAGTGCAGTGGATGATCGTGACAGTGACTACCGCAGTGAAACG
AGCAACAGCATCCCGCCGCCCTATTATACTACGTCACAACCCAACGCCTCAGTCCACCAATATTCTGTTC
GCCCACCACCCCTGGGCTCCCGGGAGTCCTACAGTGACTCCATGCACAGTTACGAGGAGTTCTCTGAGCC
ACAAGCCCTCAGCCCCACGGGTAGCAGCCGCTATGCCTCTTCCGGGGAGCTGAGCCAGGGAAGCTCTCAG
CTGAGCGAGGACTTCGACCCTGACGAGCACAGCCTGCAGGGCTCCGACATGGAGGATGAGCGGGACCGGG
ACTCCTACCACTCCTGCCACAGCTCGGTCAGCTACCACAAAGACTCGCCTCGCTGGGACCAGGATGAGGA
AGAGCTGGAGGAGGACCTGGAGGACTTCCTGGAGGAGGAGGAGCTGCCTGAAGATGAGGAGGAGCTGGAG
GAGGAGGAGGAGGAGGTGCCTGACGATTTGGGCAGCTATGCCCAGCGTGAAGACGTAGCTGTGGCTGAGC
CCAAAGACTTCAAACGCATCAGCCTCCCGCCAGCTGCCCCAGGGAAGGAGGACAAGGCCCCAGTGGCACC
CACCGAGGCCCCCGACATGGCCAAGGTGGCCCCCAAGCCAGCCACGCCCGACAAGGTGCCTGCAGCTGAG
CAGATCCCTGAGGCTGAGCCACCCAAGGACGAGGAGAGTTTCAGGCCGAGAGAGGATGAGGAAGGCCAGG
AGGGGCAGGACTCCATGTCCAGGGCCAAGGCCAACTGGCTGCGTGCCTTCAACAAGGTGCGGATGCAGCT
GCAGGAGGCCCGGGGAGAAGGAGAGATGTCTAAATCCCTATGGTTCAAAGGCGGCCCAGGGGGCGGTCTC
ATCATCATCGACAGCATGCCAGACATCCGCAAGAGGAAACCTATCCCACTCGTGAGCGACTTGTCCCTGG
TCCAGTCCAGGAAAGCGGGCATCACCTCGGCCTTGGCCTCCAGCACGTTGAACAACGAGGAGCTGAAAAA
CCACGTTTACAAGAAGACCCTGCAAGCCTTAATCTACCCCATCTCGTGCACGACGCCACACAACTTCGAA
GTGTGGACGGCCACCACGCCCACCTACTGCTACGAGTGCGAGGGGCTGCTGTGGGGCATCGCGAGGCAGG
GCATGCGCTGCACCGAGTGCGGTGTCAAGTGCCACGAGAAGTGCCAGGACCTGCTCAACGCCGACTGCCT
GCAGCGGGCTGCGGAGAAGAGCTCCAAGCACGGGGCGGAGGACCGGACACAGAACATCATCATGGTGCTC
AAGGACCGCATGAAGATCCGGGAGCGCAACAAGCCCGAGATCTTCGAGCTCATCCAGGAGATCTTCGCGG
TGACCAAGACGGCGCACACGCAGCAGATGAAGGCGGTCAAGCAGAGCGTGCTGGACGGCACGTCCAAGTG
GTCCGCCAAGATCAGCATCACCGTGGTCTGCGCCCAGGGCTTGCAGGCAAAGGACAAGACAGGATCCAGT
GACCCCTATGTCACCGTCCAGGTCGGGAAGACCAAGAAACGGACAAAAACCATCTATGGGAACCTCAACC
CGGTGTGGGAGGAGAATTTCCACTTTGAATGTCACAATTCCTCCGACCGCATCAAGGTGCGCGTCTGGGA
CGAGGATGACGACATCAAATCCCGCGTGAAACAGAGGTTCAAGAGGGAATCTGACGATTTCCTGGGGCAG
ACGATCATTGAGGTGCGGACGCTCAGCGGCGAGATGGACGTGTGGTACAACCTGGACAAGCGAACTGACA
AATCTGCCGTGTCGGGTGCCATCCGGCTCCACATCAGTGTGGAGATCAAAGGCGAGGAGAAGGTGGCCCC
GTACCATGTCCAGTACACCTGTCTGCATGAGAACCTGTTCCACTTCGTGACCGACGTGCAGAACAATGGG
GTCGTGAAGATCCCAGATGCCAAGGGTGACGATGCCTGGAAGGTTTACTACGATGAGACAGCCCAGGAGA
TTGTGGACGAGTTTGCCATGCGCTACGGCGTCGAGTCCATCTACCAAGCCATGACCCACTTTGCCTGCCT
CTCCTCCAAGTATATGTGCCCAGGGGTGCCTGCCGTCATGAGCACCCTGCTCGCCAACATCAATGCCTAC
TACGCACACACCACCGCCTCCACCAACGTGTCTGCCTCCGACCGCTTCGCCGCCTCCAACTTTGGGAAAG
AGCGCTTCGTGAAACTCCTGGACCAGCTGCATAACTCCCTGCGGATTGACCTCTCCATGTACCGGAATAA
CTTCCCAGCCAGCAGCCCGGAGAGACTCCAGGACCTCAAATCCACTGTGGACCTTCTCACCAGCATCACC
TTCTTTCGGATGAAGGTACAAGAACTCCAGAGCCCGCCCCGAGCCAGCCAGGTGGTAAAGGACTGTGTGA
AAGCCTGCCTTAATTCTACCTACGAGTACATCTTCAATAACTGCCATGAACTGTACAGCCGGGAGTACCA
GACAGACCCGGCCAAGAAGGGGGAAGTTCTCCCAGAGGAACAGGGGCCCAGCATCAAGAACCTCGACTTC
TGGTCCAAGCTGATTACCCTCATAGTGTCCATCATTGAGGAAGACAAGAATTCCTACACTCCCTGCCTCA
ACCAGTTTCCCCAGGAGCTGAATGTGGGTAAAATCAGCGCTGAAGTGATGTGGAATCTGTTTGCCCAAGA
CATGAAGTACGCCATGGAGGAGCACGACAAGCATCGTCTATGCAAGAGTGCCGACTACATGAACCTCCAC
TTCAAGGTGAAATGGCTCTACAATGAGTATGTGACGGAACTTCCCGCCTTCAAGGACCGCGTGCCTGAGT
ACCCTGCATGGTTTGAACCCTTCGTCATCCAGTGGCTGGATGAGAATGAGGAGGTGTCCCGGGATTTCCT
GCACGGTGCCCTGGAGCGAGACAAGAAGGATGGGTTCCAGCAGACCTCAGAGCATGCCCTATTCTCCTGC
TCCGTGGTGGATGTTTTCTCCCAACTCAACCAGAGCTTTGAAATCATCAAGAAACTCGAGTGTCCCGACC
CTCAGATCGTGGGGCACTACATGAGGCGCTTTGCCAAGACCATCAGTAATGTGCTCCTCCAGTATGCAGA
CATCATCTCCAAGGACTTTGCCTCCTACTGCTCCAAGGAGAAGGAGAAAGTGCCCTGCATTCTCATGAAT
AACACTCAACAGCTACGAGTTCAGCTGGAGAAGATGTTCGAAGCCATGGGAGGAAAGGAGCTGGATGCTG
AAGCCAGTGACATCCTGAAGGAGCTTCAGGTGAAACTCAATAACGTCTTGGATGAGCTCAGCCGGGTGTT
TGCTACCAGCTTCCAGCCGCACATTGAAGAGTGTGTCAAACAGATGGGTGACATCCTTAGCCAGGTTAAG
GGCACAGGCAATGTGCCAGCCAGTGCCTGCAGCAGCGTGGCCCAGGACGCGGACAATGTGTTGCAGCCCA
TCATGGACCTGCTGGACAGCAACCTGACCCTCTTTGCCAAAATCTGTGAGAAGACTGTGCTGAAGCGAGT
GCTGAAGGAGCTGTGGAAGCTGGTTATGAACACCATGGAGAAAACCATCGTCCTGCCGCCCCTCACTGAC
CAGACGGGGAACCTCTTGAGAAAACATGGCAAGGGATTAGAAAAGGGCAGGGTGAAATTGCCAAGCCACT
CAGACGGAACCCAGATGATCTTCAATGCAGCCAAGGAGCTGGGTCAGCTGTCCAAACTCAAGGATCACAT
GGTACGAGAAGAAGCCAAGAGCTTGACCCCAAAGCAGTGCGCGGTTGTTGAGTTGGCCCTGGACACCATC
AAGCAATATTTCCACGCGGGTGGCGTGGGCCTCAAGAAGACCTTCCTGGAGAAGAGCCCGGACCTGCAAT
CCTTGCGCTATGCCCTGTCGCTCTACACGCAGGCCACCGACCTGCTAATCAAGACCTTTGTACAGACGCA
ATCGGCCCAGGGCTTGGGTGTAGAAGACCCTGTGGGTGAAGTCTCTGTCCATGTTGAGCTGTTCACTCAT
CCAGGAACTGGGGAACACAAGGTCACAGTGAAAGTGGTGGCTGCCAATGACCTCAAGTGGCAGACTTCTG
GCATCTTCCGGCCGTTCATCGAGGTCAACATCATTGGGCCCCAGCTCAGCGACAAGAAACGCAAGTTTGC
GACCAAATCCAAGAACAATAGCTGGGCTCCCAAGTACAATGAGAGCTTCCAGTTCACGCTGAGCGCCGAC
GCGGGTCCCGAGTGCTATGAGCTGCAGGTGTGCGTCAAGGACTACTGCTTCGCGCGCGAGGACCGCACGG
TGGGGCTGGCCGTGCTGCAGCTGCGTGAGCTGGCCCAGCGCGGGAGCGCCGCCTGCTGGCTGCCGCTCGG
CCGCCGCATCCACATGGACGACACGGGCCTCACGGTGCTGCGAATCCTCTCGCAGCGCAGCAACGACGAG
GTGGCCAAGGAGTTCGTGAAGCTCAAGTCGGACACGCGCTCCGCCGAGGAGGGCGGTGCCGCGCCTGCGC
CTTAGCGCGGGCGGTCGGCCGAGCGGCACTGCGCCTGCGCGGAGGGCGCTGGGGGGGGAGGGACGGGGCT
TGCGCCTTGGTGGGACCTCCCCAGGGGCGGGGCTCGGGGGGCTCCACGCCAAGGGTGGGCTGCGCCTACG
CCCTTGACTCAGCTTTCCCTTTTGGGGAATTAGGAATGGAGGATGCCCCGCCCTCTCGGGAGGCCACGCC
CAAGGGCGCGACGAAGGAAGGAGCCACATCCCCAACTTGAGGCCACGCCCCCAGCACCTAGGGGGCATTT
TGAGCTGGGATGGGGGAAACCTCGTCCCTATGGAGGAGGCCACATCCCGGGGCTCTGGTACCGGGAGGCA
CCACCTCATGTCCCCTGGAAAAGCCATAAGATGGGACCCAGACCCCTGGGACCCCAGACCAATTGCCAAG
TATGGAAATCTCAGCTCCCTCGAGGGGGGGCCCTGGGCAAGGGGTAGGGCTCTCTGGAGCGCCCCTCTAG
GTGGCCTGGGGACTGGAGGGACCAGGATGCTGGTTGGAGGGCCCCGGAATACCGGAGTCCCTTTAGATAT
TTGTGCAAAAAATAAATGGGGGGAGGGGGGAGGATGGGATTTCAAAAGCACATGCGCCCTTGGGCGCCCA
AACCCTGGGGGCCGAGGGGACGGCTCTGGTTCCCCACGCTGCCCCTACTTCCCTTTGGGAGTTTGCCTCT
CCCTCTCCCCCAACAAACCCAGTCCTCATATCATAGAGTTCAACACACCCATTTGACAGATGGCAAAACT
GAGGCTTAAAGAGCTGCTTGAGACTTGGCCAAGGTTCCAGGTGCCATACCCTCTGTGCCCCTCCCTTAGG
CCTGTGTGCCCCATGGAAGGGTGGGCTGAGATCGGGATGACCTGACACAGCTCCCTATTGCTGCTAATTC
CCCCTCGGCCTCCTCCAAGGGGTGGGAATTCCAGGCCAAGACCCCTACTTCGCCTTTCCTTCTCCGGCTG
CCAAGCAGGACCTTTGCCCTCAGCCCTTTCTCCTGGGATCTCCATGGGGGATGCCATGAGGGCCTCCCAC
CACAAAAGAGAATTTGGGATCCCCTGGTCCCAGGTTTCTCCATCCCTTCTTCCTTTTCCAGAATTTTCCA
AATAGGAAAGAACAGAAGGAGACCAGAAACTCTAGGGGGGAGAAAGAGAATGAGAGAAAGAGAATGAGAG
AGAGAGAAACACAAACACAGTGACACAGTGAGAGCTTAGTCTCCAAGAGCCTATTCATTGATTCAAACAC
CCAAGCCACAGGATACCTCAGATGGCCCTCTTGCCAGCTGGAAGCTCTTTCTCCAATGAGCAAAGTTACA
GTGACCTGGCTGGAGTTACCTGGTGCACATAGGACCTTAGGGGAAAGTTCAGCGTGGACTACACTTGCTC
TGGGATCTGCTTTTCCACATGTGTGTATGGCACGCCTTTTTCTGCTGGATTGGGAAGGACAAGATTTTGC
TGTGCTAGGGAGAAATGAAAACGGGGTGAGCTGAGTAGCTGGGTTTCTGGAGGATAGAACATCAGATGGG
GAGGCTTTCCGAGGTGAAGAATGAGAGGGAACCACTTACTAGAGAGAAAAGAGCTCCAGGCCTGGGGAAC
AGCACGTGCGAAGGCCAGGAGAGAAGAACTGTTGAAACAACGAGAAGGGTGGCACGGCTGGAGCTGAGCC
AGCAAGGGGGATCGTGAGGAGCCTTGGGGTTGGGGAGATCTGCAGAAGCATCAGACCAGGCAGGGCCTCG
TACGCAGTCCTGAGGAGTTTTACTTTTATTCTAAGACAGTTGGGGAGCTCCAGGAGCTGTTTTAAGTTGG
GGAGAGACTGGATTCCAGCCTGCAAAAGCTGTTTTGTGAAGACTAAAACCAGTGAGGAGAGGTGGAGGTG
CTTTGGGGACACTGAAATGGATTCTTGGAAAGATTCTGAAGGCTGTGTTGAAAAGACACCTATAGCTGTG
GGGACATGACTATAATCCCAGCATTTGGGGAGACCGAGGCTGGCAGATCACTTAAGGTCAGGAGTTTGAG
ACCAGCCTGGCCAACATGGCGAAACCCCATCTCTGCTAAAAATACAAAAATTAGCTGGGTGCAGTGGTGC
ATGCCTGTAGTCCCAGCTACTCAGGAGACTGAGGCGGGAGAATTGTTTGAACCCTGGAGGCAGAGGTTGT
AGTGAGTCGTGATCACACAACTGCACTCCAGCCTGGGCAACAGAACAATACTCCATTCCCTCCCCTCTAC
CCCACCAAAAAAAAAAAAAAATCCTGCCCTTAGATGAGCTCTAGGGCTGCTGAGTACAGTTGTCCCAGTT
GCACAGTGCCCAAGGGTTTGGCATTGCTAAGAAGGCCACGTGCAAATCCTAGATATTGAGTGTTGTATGT
TTGTGACGTTGGTTTCCCGACATGTGAATGGCCCAAGTGTCTGGAAGAAGTGGCGCCACTTTCTAATTTG
CTTGGAGATGTTGCATGTCCCTTAAATTCAGACAGGTGCAGGTAACTGGAGGTTCTGAACCAAAGGTTAA
AATGCAAATTCTCATACAGGGTTGGGAAGTTGTAGCCAGGGATAAGCTTATGTGACTGTTATATGGACTG
AGGAGCAGATGTGAATTTCGAACCATGACATGGCTGAGGGTAGGGGTCGGGTGGATGGATGATTCAGGGT
TGTAACCCATAGAGCCCAAAGGGGAAGTGATCTGTGACCTGGGGTGAGGGTGATCTGGAAGATTTTTGGA
TGGCTGGAAAGAAATGGGGAAGTCGAGCTGCCTGAGAGAGCCAAGTTATTTCCCAAAAGATTCCTTAGGA
GTCTTTCTGTTCAAGACCTCCGTGTGTGTGTGTGTGTGTTTAGGGTTCCCCAGCAATGGCCCAGGCATGT
GAAGGAAACAAGCTTCTTCAGGGAATATTTGTTGAATGAGTTTTCCTGACTCCCAGGCTAGAACTGTTTT
TGCAATTTCCACCCTCTTTTCTTTCCCCCAGAGAACTCCTATTCGTCCTTCAAAACCCATCACGGAAACC
CCTCTTGGAGAAAACCCTCCTTCCTTCCCCTCAGGACTTTCCCAGCCACCGTCTCTCCTCCAGTCCAGCC
TGATGCCATGGGACTGGGGGTTTCTCTGTCCAGCTCTGTTTCTCCCAGACTGGGGTCTGAGGACTCTCAG
GACCCCCAACTTTACCTAGCACAGGCTGGGCACAAGTGGGTGACAGGGAGTCTACGCCTAGTGGAATTAT
GTATTGGGGCAGGGTCAGTGTGAGAATACACATCCGCATGCATGTCTGTCCATGTCTGTCCGTACCAACC
TTCCCCTTCCACACGGACCTGGGCACATAGGAGGTGTCTGAGCCTGACACATGGGACAGAGAGTGGACAT
GGCTGAGACACGGACAGAGAAAAGACAAGGAGTCCAGGGGGCTGAAAGCCTTTTGAAATCAGGAAGTTCC
TGTATTGGCAGAACAAAGCCCAGAGAGGAGCAGGGCTTTCCTCAACGCCACCCAGCAAGTGGACACAGAG
CCCGGCCTTGGATGACACCTCCAGGGTTCTGAACCCTGGACCTCGCTTTATGCAAGGAGCTGGCCCCACA
TTTCCATGAATCGGGGAAACAGCACAAGAAGGTTGGCCTGTGGCAGGGCAAGGGTTAAAGGGGTGACATT
GAGGGATGCCTCAGAGTCAAAGTCCCCTGACCAAGAGGAATAGAGTAGAAAACACAGAGACAGAGGGTGA
GATCACGCCCCGATGAGGACGGAGAGAGACAGAGATGGAGAGAGACATAGAGGTGGAAATATACAGAGAA
AGATAAATGCAGAGACCAAGGCAGGGAGTGTCGGGGGAAGTAAAGAGGGTGTCCTGAAGAAAGAAGGATC
TGTTCACTCTTACCAGTCTGTCCTCGAATGATTTGCATAAAATGAGGAGGTGCCTGTCCACACCCCCAAT
TCCTCTCTCAGGCCCCAGAGCCTGAGACCTCACCATGCCCCCATCAGAGATGCAAAAAACTAAACACCCA
ACTAGAAATCCTTGGGACCTCTCTCGGCTGGGATCTCAGAGCCTTTCTGTCCCCTACCCCTACCCCATGT
GCTGTCGATTTTGCAGATGGGGACAACCTGGGGCCTCCCGGAACTCTGCCACCCTGGGGAAGTTGGGGGA
GGGCCTTAGTCCCGGATCACAACCCCGTCTGCTCCCCAGAATCCTTTCCTAAGAATCGTTGAGGACCAAA
GTTGTCTTTGCTGACACGTGTTGCTTTTCTCTTTGCCTTTTATTGTTTCAGAGAAAAATCAAGTTGACTG
TGTCAAGTAACACCCCACCCCTTACCCCCGTCCAGCCATAGTGGCTCTCTGGAGACACAGGTCACAGGCG
GAGGGTCCCCTGATCATCCCCAACCACACAGCCAGGGGGACTTGACCCCTGTCCACCCCTGTCTCGTGCT
CCCTCAGACCCCCACAAACCGGCCAAGCAGTCCGGGGAGGCTTCCCCTCCACACAACTCTTAGCATGTGA
TTGCAGATGTGAAATCAAAACGTTGTTTGTTTTTTGTTTTGTTTTGATTCTACCCCGTCGGTCCAGTGTC
TGCACAGACGCCTTCATTTCTCTGTAAATATGTGACTTGGAACAAATGTTTAACACAAACGAGAAGTGGT
CATGAATGCATGGTGTTGAGATGTTTTGCACTATTCTGACTTTTTGGTCTCTGTAAAAATATTTTATTAA
CAGCAGACATTAAAAAAAGAAAAACCACACACA.

In various embodiments, an UNC13A mRNA transcript comprises the sequence provided as SEQ ID NO: 9589.

(SEQ ID NO: 9589 (SOURCE NCBI Reference Sequence NM_001387022.1)
GCCCCCGGTGCTGAACCAAGATGGCCGGTGGCGGCCGGGCCCCGGCGTGAGCCAAGCGCGGGCTGCAGCC
GGGAGATGCCCCAGCCCAGCGGCCGCTGAGCCCGACCCGACAGAGCCGGCCCGGCCGCCTCCGGCCCACC
TGCGAGCTCGGAGACATGTCTCTGCTTTGCGTTGGAGTCAAAAAAGCCAAGTTTGATGGTGCCCAAGAGA
AATTCAACACGTACGTGACCCTGAAAGTGCAGAATGTCAAGAGCACGACCATCGCGGTGCGGGGCAGCCA
GCCCAGCTGGGAGCAGGATTTCATGTTCGAGATTAACCGTCTGGATTTGGGACTGACGGTGGAGGTGTGG
AATAAGGGTCTCATCTGGGACACAATGGTGGGCACTGTGTGGATCCCACTGAGGACCATCCGCCAGTCCA
ATGAGGAGGGCCCTGGAGAGTGGCTGACGCTGGACTCCCAGGTCATCATGGCAGACAGTGAGATCTGTGG
CACCAAGGACCCCACCTTCCACCGCATCCTCCTGGACACGCGCTTTGAGCTACCCTTAGACATTCCTGAA
GAGGAGGCTCGCTACTGGGCCAAGAAGCTGGAGCAGCTCAATGCTATGCGGGACCAGGATGAATATTCGT
TCCAAGATGAGCAAGACAAGCCTCTGCCTGTCCCCAGCAACCAGTGCTGCAACTGGAATTATTTTGGCTG
GGGTGAGCAGCACAACGATGACCCCGACAGTGCAGTGGATGATCGTGACAGTGACTACCGCAGTGAAACG
AGCAACAGCATCCCGCCGCCCTATTATACTACGTCACAACCCAACGCCTCAGTCCACCAATATTCTGTTC
GCCCACCACCCCTGGGCTCCCGGGAGTCCTACAGTGACTCCATGCACAGTTACGAGGAGTTCTCTGAGCC
ACAAGCCCTCAGCCCCACGGGTAGCAGCCGCTATGCCTCTTCCGGGGAGCTGAGCCAGGGAAGCTCTCAG
CTGAGCGAGGACTTCGACCCTGACGAGCACAGCCTGCAGGGCTCCGACATGGAGGATGAGCGGGACCGGG
ACTCCTACCACTCCTGCCACAGCTCGGTCAGCTACCACAAAGACTCGCCTCGCTGGGACCAGGATGAGGA
AGAGCTGGAGGAGGACCTGGAGGACTTCCTGGAGGAGGAGGAGCTGCCTGAAGATGAGGAGGAGCTGGAG
GAGGAGGAGGAGGAGGTGCCTGACGATTTGGGCAGCTATGCCCAGCGTGAAGACGTAGCTGTGGCTGAGC
CCAAAGACTTCAAACGCATCAGCCTCCCGCCAGCTGCCCCAGGGAAGGAGGACAAGGCCCCAGTGGCACC
CACCGAGGCCCCCGACATGGCCAAGGTGGCCCCCAAGCCAGCCACGCCCGACAAGGTGCCTGCAGCTGAG
CAGATCCCTGAGGCTGAGCCACCCAAGGACGAGGAGAGTTTCAGGCCGAGAGAGGATGAGGAAGGCCAGG
AGGGGCAGGACTCCATGTCCAGGGCCAAGGCCAACTGGCTGCGTGCCTTCAACAAGGTGCGGATGCAGCT
GCAGGAGGCCCGGGGAGAAGGAGAGATGTCTAAATCCCTATGGTTCAAAGGCGGCCCAGGGGGCGGTCTC
ATCATCATCGACAGCATGCCAGACATCCGCAAGAGGAAACCTATCCCACTCGTGAGCGACTTGTCCCTGG
TCCAGTCCAGGAAAGCGGGCATCACCTCGGCCTTGGCCTCCAGCACGTTGAACAACGAGGAGCTGAAAAA
CCACGTTTACAAGAAGACCCTGCAAGCCTTAATCTACCCCATCTCGTGCACGACGCCACACAACTTCGAA
GTGTGGACGGCCACCACGCCCACCTACTGCTACGAGTGCGAGGGGCTGCTGTGGGGCATCGCGAGGCAGG
GCATGCGCTGCACCGAGTGCGGTGTCAAGTGCCACGAGAAGTGCCAGGACCTGCTCAACGCCGACTGCCT
GCAGCGGGCTGCGGAGAAGAGCTCCAAGCACGGGGCGGAGGACCGGACACAGAACATCATCATGGTGCTC
AAGGACCGCATGAAGATCCGGGAGCGCAACAAGCCCGAGATCTTCGAGCTCATCCAGGAGATCTTCGCGG
TGACCAAGACGGCGCACACGCAGCAGATGAAGGCGGTCAAGCAGAGCGTGCTGGACGGCACGTCCAAGTG
GTCCGCCAAGATCAGCATCACCGTGGTCTGCGCCCAGGGCTTGCAGGCAAAGGACAAGACAGGATCCAGT
GACCCCTATGTCACCGTCCAGGTCGGGAAGACCAAGAAACGGACAAAAACCATCTATGGGAACCTCAACC
CGGTGTGGGAGGAGAATTTCCACTTTGAATGTCACAATTCCTCCGACCGCATCAAGGTGCGCGTCTGGGA
CGAGGATGACGACATCAAATCCCGCGTGAAACAGAGGTTCAAGAGGGAATCTGACGATTTCCTGGGGCAG
ACGATCATTGAGGTGCGGACGCTCAGCGGCGAGATGGACGTGTGGTACAACCTGGACAAGCGAACTGACA
AATCTGCCGTGTCGGGTGCCATCCGGCTCCACATCAGTGTGGAGATCAAAGGCGAGGAGAAGGTGGCCCC
GTACCATGTCCAGTACACCTGTCTGCATGAGAACCTGTTCCACTTCGTGACCGACGTGCAGAACAATGGG
GTCGTGAAGATCCCAGATGCCAAGGGTGACGATGCCTGGAAGGTTTACTACGATGAGACAGCCCAGGAGA
TTGTGGACGAGTTTGCCATGCGCTACGGCGTCGAGTCCATCTACCAAGCCATGACCCACTTTGCCTGCCT
CTCCTCCAAGTATATGTGCCCAGGGGTGCCTGCCGTCATGAGCACCCTGCTCGCCAACATCAATGCCTAC
TACGCACACACCACCGCCTCCACCAACGTGTCTGCCTCCGACCGCTTCGCCGCCTCCAACTTTGGGAAAG
AGCGCTTCGTGAAACTCCTGGACCAGCTGCATAACTCCCTGCGGATTGACCTCTCCATGTACCGGAATAA
CTTCCCAGCCAGCAGCCCGGAGAGACTCCAGGACCTCAAATCCACTGTGGACCTTCTCACCAGCATCACC
TTCTTTCGGATGAAGGTACAAGAACTCCAGAGCCCGCCCCGAGCCAGCCAGGTGGTAAAGGACTGTGTGA
AAGCCTGCCTTAATTCTACCTACGAGTACATCTTCAATAACTGCCATGAACTGTACAGCCGGGAGTACCA
GACAGACCCGGCCAAGAAGGGGGAAGTTCTCCCAGAGGAACAGGGGCCCAGCATCAAGAACCTCGACTTC
TGGTCCAAGCTGATTACCCTCATAGTGTCCATCATTGAGGAAGACAAGAATTCCTACACTCCCTGCCTCA
ACCAGTTTCCCCAGGAGCTGAATGTGGGTAAAATCAGCGCTGAAGTGATGTGGAATCTGTTTGCCCAAGA
CATGAAGTACGCCATGGAGGAGCACGACAAGCATCGTCTATGCAAGAGTGCCGACTACATGAACCTCCAC
TTCAAGGTGAAATGGCTCTACAATGAGTATGTGACGGAACTTCCCGCCTTCAAGGACCGCGTGCCTGAGT
ACCCTGCATGGTTTGAACCCTTCGTCATCCAGTGGCTGGATGAGAATGAGGAGGTGTCCCGGGATTTCCT
GCACGGTGCCCTGGAGCGAGACAAGAAGGATGGGTTCCAGCAGACCTCAGAGCATGCCCTATTCTCCTGC
TCCGTGGTGGATGTTTTCTCCCAACTCAACCAGAGCTTTGAAATCATCAAGAAACTCGAGTGTCCCGACC
CTCAGATCGTGGGGCACTACATGAGGCGCTTTGCCAAGACCATCAGTAATGTGCTCCTCCAGTATGCAGA
CATCATCTCCAAGGACTTTGCCTCCTACTGCTCCAAGGAGAAGGAGAAACCCTGCATTCTCATGAATAAC
ACTCAACAGCTACGAGTTCAGCTGGAGAAGATGTTCGAAGCCATGGGAGGAAAGGAGCTGGATGCTGAAG
CCAGTGACATCCTGAAGGAGCTTCAGGTGAAACTCAATAACGTCTTGGATGAGCTCAGCCGGGTGTTTGC
TACCAGCTTCCAGCCGCACATTGAAGAGTGTGTCAAACAGATGGGTGACATCCTTAGCCAGGTTAAGGGC
ACAGGCAATGTGCCAGCCAGTGCCTGCAGCAGCGTGGCCCAGGACGCGGACAATGTGTTGCAGCCCATCA
TGGACCTGCTGGACAGCAACCTGACCCTCTTTGCCAAAATCTGTGAGAAGACTGTGCTGAAGCGAGTGCT
GAAGGAGCTGTGGAAGCTGGTTATGAACACCATGGAGAAAACCATCGTCCTGCCGCCCCTCACTGACCAG
ACGGGGAACCTCTTGAGAAAACATGGCAAGGGATTAGAAAAGGGCAGGGTGAAATTGCCAAGCCACTCAG
ACGGAACCCAGATGATCTTCAATGCAGCCAAGGAGCTGGGTCAGCTGTCCAAACTCAAGGATCACATGGT
ACGAGAAGAAGCCAAGAGCTTGACCCCAAAGCAGTGCGCGGTTGTTGAGTTGGCCCTGGACACCATCAAG
CAATATTTCCACGCGGGTGGCGTGGGCCTCAAGAAGACCTTCCTGGAGAAGAGCCCGGACCTGCAATCCT
TGCGCTATGCCCTGTCGCTCTACACGCAGGCCACCGACCTGCTAATCAAGACCTTTGTACAGACGCAATC
GGCCCAGGGCTTGGGTGTAGAAGACCCTGTGGGTGAAGTCTCTGTCCATGTTGAGCTGTTCACTCATCCA
GGAACTGGGGAACACAAGGTCACAGTGAAAGTGGTGGCTGCCAATGACCTCAAGTGGCAGACTTCTGGCA
TCTTCCGGCCGTTCATCGAGGTCAACATCATTGGGCCCCAGCTCAGCGACAAGAAACGCAAGTTTGCGAC
CAAATCCAAGAACAATAGCTGGGCTCCCAAGTACAATGAGAGCTTCCAGTTCACGCTGAGCGCCGACGCG
GGTCCCGAGTGCTATGAGCTGCAGGTGTGCGTCAAGGACTACTGCTTCGCGCGCGAGGACCGCACGGTGG
GGCTGGCCGTGCTGCAGCTGCGTGAGCTGGCCCAGCGCGGGAGCGCCGCCTGCTGGCTGCCGCTCGGCCG
CCGCATCCACATGGACGACACGGGCCTCACGGTGCTGCGAATCCTCTCGCAGCGCAGCAACGACGAGGTG
GCCAAGGAGTTCGTGAAGCTCAAGTCGGACACGCGCTCCGCCGAGGAGGGCGGTGCCGCGCCTGCGCCTT
AGCGCGGGCGGTCGGCCGAGCGGCACTGCGCCTGCGCGGAGGGCGCTGGGCGGGGAGGGACGGGGCTTGC
GCCTTGGTGGGACCTCCCCAGGGGCGGGGCTCGGGGGGCTCCACGCCAAGGGTGGGCTGCGCCTACGCCC
TTGACTCAGCTTTCCCTTTTGGGGAATTAGGAATGGAGGATGCCCCGCCCTCTCGGGAGGCCACGCCCAA
GGGCGCGACGAAGGAAGGAGCCACATCCCCAACTTGAGGCCACGCCCCCAGCACCTAGGGGGCATTTTGA
GCTGGGATGGGGGAAACCTCGTCCCTATGGAGGAGGCCACATCCCGGGGCTCTGGTACCGGGAGGCACCA
CCTCATGTCCCCTGGAAAAGCCATAAGATGGGACCCAGACCCCTGGGACCCCAGACCAATTGCCAAGTAT
GGAAATCTCAGCTCCCTCGAGGGGGGGCCCTGGGCAAGGGGTAGGGCTCTCTGGAGCGCCCCTCTAGGTG
GCCTGGGGACTGGAGGGACCAGGATGCTGGTTGGAGGGCCCCGGAATACCGGAGTCCCTTTAGATATTTG
TGCAAAAAATAAATGGGGGGAGGGGGGAGGATGGGATTTCAAAAGCACATGCGCCCTTGGGCGCCCAAAC
CCTGGGGGCCGAGGGGACGGCTCTGGTTCCCCACGCTGCCCCTACTTCCCTTTGGGAGTTTGCCTCTCCC
TCTCCCCCAACAAACCCAGTCCTCATATCATAGAGTTCAACACACCCATTTGACAGATGGCAAAACTGAG
GCTTAAAGAGCTGCTTGAGACTTGGCCAAGGTTCCAGGTGCCATACCCTCTGTGCCCCTCCCTTAGGCCT
GTGTGCCCCATGGAAGGGTGGGCTGAGATCGGGATGACCTGACACAGCTCCCTATTGCTGCTAATTCCCC
CTCGGCCTCCTCCAAGGGGTGGGAATTCCAGGCCAAGACCCCTACTTCGCCTTTCCTTCTCCGGCTGCCA
AGCAGGACCTTTGCCCTCAGCCCTTTCTCCTGGGATCTCCATGGGGGATGCCATGAGGGCCTCCCACCAC
AAAAGAGAATTTGGGATCCCCTGGTCCCAGGTTTCTCCATCCCTTCTTCCTTTTCCAGAATTTTCCAAAT
AGGAAAGAACAGAAGGAGACCAGAAACTCTAGGGGGGAGAAAGAGAATGAGAGAAAGAGAATGAGAGAGA
GAGAAACACAAACACAGTGACACAGTGAGAGCTTAGTCTCCAAGAGCCTATTCATTGATTCAAACACCCA
AGCCACAGGATACCTCAGATGGCCCTCTTGCCAGCTGGAAGCTCTTTCTCCAATGAGCAAAGTTACAGTG
ACCTGGCTGGAGTTACCTGGTGCACATAGGACCTTAGGGGAAAGTTCAGCGTGGACTACACTTGCTCTGG
GATCTGCTTTTCCACATGTGTGTATGGCACGCCTTTTTCTGCTGGATTGGGAAGGACAAGATTTTGCTGT
GCTAGGGAGAAATGAAAACGGGGTGAGCTGAGTAGCTGGGTTTCTGGAGGATAGAACATCAGATGGGGAG
GCTTTCCGAGGTGAAGAATGAGAGGGAACCACTTACTAGAGAGAAAAGAGCTCCAGGCCTGGGGAACAGC
ACGTGCGAAGGCCAGGAGAGAAGAACTGTTGAAACAACGAGAAGGGTGGCACGGCTGGAGCTGAGCCAGC
AAGGGGGATCGTGAGGAGCCTTGGGGTTGGGGAGATCTGCAGAAGCATCAGACCAGGCAGGGCCTCGTAC
GCAGTCCTGAGGAGTTTTACTTTTATTCTAAGACAGTTGGGGAGCTCCAGGAGCTGTTTTAAGTTGGGGA
GAGACTGGATTCCAGCCTGCAAAAGCTGTTTTGTGAAGACTAAAACCAGTGAGGAGAGGTGGAGGTGCTT
TGGGGACACTGAAATGGATTCTTGGAAAGATTCTGAAGGCTGTGTTGAAAAGACACCTATAGCTGTGGGG
ACATGACTATAATCCCAGCATTTGGGGAGACCGAGGCTGGCAGATCACTTAAGGTCAGGAGTTTGAGACC
AGCCTGGCCAACATGGCGAAACCCCATCTCTGCTAAAAATACAAAAATTAGCTGGGTGCAGTGGTGCATG
CCTGTAGTCCCAGCTACTCAGGAGACTGAGGCGGGAGAATTGTTTGAACCCTGGAGGCAGAGGTTGTAGT
GAGTCGTGATCACACAACTGCACTCCAGCCTGGGCAACAGAACAATACTCCATTCCCTCCCCTCTACCCC
ACCAAAAAAAAAAAAAAATCCTGCCCTTAGATGAGCTCTAGGGCTGCTGAGTACAGTTGTCCCAGTTGCA
CAGTGCCCAAGGGTTTGGCATTGCTAAGAAGGCCACGTGCAAATCCTAGATATTGAGTGTTGTATGTTTG
TGACGTTGGTTTCCCGACATGTGAATGGCCCAAGTGTCTGGAAGAAGTGGCGCCACTTTCTAATTTGCTT
GGAGATGTTGCATGTCCCTTAAATTCAGACAGGTGCAGGTAACTGGAGGTTCTGAACCAAAGGTTAAAAT
GCAAATTCTCATACAGGGTTGGGAAGTTGTAGCCAGGGATAAGCTTATGTGACTGTTATATGGACTGAGG
AGCAGATGTGAATTTCGAACCATGACATGGCTGAGGGTAGGGGTCGGGTGGATGGATGATTCAGGGTTGT
AACCCATAGAGCCCAAAGGGGAAGTGATCTGTGACCTGGGGTGAGGGTGATCTGGAAGATTTTTGGATGG
CTGGAAAGAAATGGGGAAGTCGAGCTGCCTGAGAGAGCCAAGTTATTTCCCAAAAGATTCCTTAGGAGTC
TTTCTGTTCAAGACCTCCGTGTGTGTGTGTGTGTGTTTAGGGTTCCCCAGCAATGGCCCAGGCATGTGAA
GGAAACAAGCTTCTTCAGGGAATATTTGTTGAATGAGTTTTCCTGACTCCCAGGCTAGAACTGTTTTTGC
AATTTCCACCCTCTTTTCTTTCCCCCAGAGAACTCCTATTCGTCCTTCAAAACCCATCACGGAAACCCCT
CTTGGAGAAAACCCTCCTTCCTTCCCCTCAGGACTTTCCCAGCCACCGTCTCTCCTCCAGTCCAGCCTGA
TGCCATGGGACTGGGGGTTTCTCTGTCCAGCTCTGTTTCTCCCAGACTGGGGTCTGAGGACTCTCAGGAC
CCCCAACTTTACCTAGCACAGGCTGGGCACAAGTGGGTGACAGGGAGTCTACGCCTAGTGGAATTATGTA
TTGGGGCAGGGTCAGTGTGAGAATACACATCCGCATGCATGTCTGTCCATGTCTGTCCGTACCAACCTTC
CCCTTCCACACGGACCTGGGCACATAGGAGGTGTCTGAGCCTGACACATGGGACAGAGAGTGGACATGGC
TGAGACACGGACAGAGAAAAGACAAGGAGTCCAGGGGGCTGAAAGCCTTTTGAAATCAGGAAGTTCCTGT
ATTGGCAGAACAAAGCCCAGAGAGGAGCAGGGCTTTCCTCAACGCCACCCAGCAAGTGGACACAGAGCCC
GGCCTTGGATGACACCTCCAGGGTTCTGAACCCTGGACCTCGCTTTATGCAAGGAGCTGGCCCCACATTT
CCATGAATCGGGGAAACAGCACAAGAAGGTTGGCCTGTGGCAGGGCAAGGGTTAAAGGGGTGACATTGAG
GGATGCCTCAGAGTCAAAGTCCCCTGACCAAGAGGAATAGAGTAGAAAACACAGAGACAGAGGGTGAGAT
CACGCCCCGATGAGGACGGAGAGAGACAGAGATGGAGAGAGACATAGAGGTGGAAATATACAGAGAAAGA
TAAATGCAGAGACCAAGGCAGGGAGTGTCGGGGGAAGTAAAGAGGGTGTCCTGAAGAAAGAAGGATCTGT
TCACTCTTACCAGTCTGTCCTCGAATGATTTGCATAAAATGAGGAGGTGCCTGTCCACACCCCCAATTCC
TCTCTCAGGCCCCAGAGCCTGAGACCTCACCATGCCCCCATCAGAGATGCAAAAAACTAAACACCCAACT
AGAAATCCTTGGGACCTCTCTCGGCTGGGATCTCAGAGCCTTTCTGTCCCCTACCCCTACCCCATGTGCT
GTCGATTTTGCAGATGGGGACAACCTGGGGCCTCCCGGAACTCTGCCACCCTGGGGAAGTTGGGGGAGGG
CCTTAGTCCCGGATCACAACCCCGTCTGCTCCCCAGAATCCTTTCCTAAGAATCGTTGAGGACCAAAGTT
GTCTTTGCTGACACGTGTTGCTTTTCTCTTTGCCTTTTATTGTTTCAGAGAAAAATCAAGTTGACTGTGT
CAAGTAACACCCCACCCCTTACCCCCGTCCAGCCATAGTGGCTCTCTGGAGACACAGGTCACAGGCGGAG
GGTCCCCTGATCATCCCCAACCACACAGCCAGGGGGACTTGACCCCTGTCCACCCCTGTCTCGTGCTCCC
TCAGACCCCCACAAACCGGCCAAGCAGTCCGGGGAGGCTTCCCCTCCACACAACTCTTAGCATGTGATTG
CAGATGTGAAATCAAAACGTTGTTTGTTTTTTGTTTTGTTTTGATTCTACCCCGTCGGTCCAGTGTCTGC
ACAGACGCCTTCATTTCTCTGTAAATATGTGACTTGGAACAAATGTTTAACACAAACGAGAAGTGGTCAT
GAATGCATGGTGTTGAGATGTTTTGCACTATTCTGACTTTTTGGTCTCTGTAAAAATATTTTATTAACAG
CAGACATTAAAAAAAGAAAAACCACACACA.

In various embodiments, an UNC13A mRNA transcript comprises the sequence provided as SEQ ID NO: 9590.

(SEQ ID NO: 9590 (SOURCE NCBI Reference Sequence NM_001387023.1))
GCCCCCGGTGCTGAACCAAGATGGCCGGTGGCGGCCGGGCCCCGGCGTGAGCCAAGCGCGGGCTGCAGCC
GGGAGATGCCCCAGCCCAGCGGCCGCTGAGCCCGACCCGACAGAGCCGGCCCGGCCGCCTCCGGCCCACC
TGCGAGCTCGGAGACATGTCTCTGCTTTGCGTTGGAGTCAAAAAAGCCAAGTTTGATGGTGCCCAAGAGA
AATTCAACACGTACGTGACCCTGAAAGTGCAGAATGTCAAGAGCACGACCATCGCGGTGCGGGGCAGCCA
GCCCAGCTGGGAGCAGGATTTCATGTTCGAGATTAACCGTCTGGATTTGGGACTGACGGTGGAGGTGTGG
AATAAGGGTCTCATCTGGGACACAATGGTGGGCACTGTGTGGATCCCACTGAGGACCATCCGCCAGTCCA
ATGAGGAGGGCCCTGGAGAGTGGCTGACGCTGGACTCCCAGGTCATCATGGCAGACAGTGAGATCTGTGG
CACCAAGGACCCCACCTTCCACCGCATCCTCCTGGACACGCGCTTTGAGCTACCCTTAGACATTCCTGAA
GAGGAGGCTCGCTACTGGGCCAAGAAGCTGGAGCAGCTCAATGCTATGCGGGACCAGGATGAATATTCGT
TCCAAGATGAGCAAGACAAGCCTCTGCCTGTCCCCAGCAACCAGTGCTGCAACTGGAATTATTTTGGCTG
GGGTGAGCAGCACAACGATGACCCCGACAGTGCAGTGGATGATCGTGACAGTGACTACCGCAGTGAAACG
AGCAACAGCATCCCGCCGCCCTATTATACTACGTCACAACCCAACGCCTCAGTCCACCAATATTCTGTTC
GCCCACCACCCCTGGGCTCCCGGGAGTCCTACAGTGACTCCATGCACAGTTACGAGGAGTTCTCTGAGCC
ACAAGCCCTCAGCCCCACGGGTAGCAGCCGCTATGCCTCTTCCGGGGAGCTGAGCCAGGGAAGCTCTCAG
CTGAGCGAGGACTTCGACCCTGACGAGCACAGCCTGCAGGGCTCCGACATGGAGGATGAGCGGGACCGGG
ACTCCTACCACTCCTGCCACAGCTCGGTCAGCTACCACAAAGACTCGCCTCGCTGGGACCAGGATGAGGA
AGAGCTGGAGGAGGACCTGGAGGACTTCCTGGAGGAGGAGGAGCTGCCTGAAGATGAGGAGGAGCTGGAG
GAGGAGGAGGAGGAGGTGCCTGACGATTTGGGCAGCTATGCCCAGCGTGAAGACGTAGCTGTGGCTGAGC
CCAAAGACTTCAAACGCATCAGCCTCCCGCCAGCTGCCCCAGGGAAGGAGGACAAGGCCCCAGTGGCACC
CACCGAGGCCCCCGACATGGCCAAGGTGGCCCCCAAGCCAGCCACGCCCGACAAGGTGCCTGCAGCTGAG
CAGATCCCTGAGGCTGAGCCACCCAAGGACGAGGAGAGTTTCAGGCCGAGAGAGGATGAGGAAGGCCAGG
AGGGGCAGGACTCCATGTCCAGGGCCAAGGCCAACTGGCTGCGTGCCTTCAACAAGGTGCGGATGCAGCT
GCAGGAGGCCCGGGGAGAAGGAGAGATGTCTAAATCCCTATGGTTCAAAGGCGGCCCAGGGGGCGGTCTC
ATCATCATCGACAGCATGCCAGACATCCGCAAGAGGAAACCTATCCCACTCGTGAGCGACTTGTCCCTGG
TCCAGTCCAGGAAAGCGGGCATCACCTCGGCCTTGGCCTCCAGCACGTTGAACAACGAGGAGCTGAAAAA
CCACGTTTACAAGAAGACCCTGCAAGCCTTAATCTACCCCATCTCGTGCACGACGCCACACAACTTCGAA
GTGTGGACGGCCACCACGCCCACCTACTGCTACGAGTGCGAGGGGCTGCTGTGGGGCATCGCGAGGCAGG
GCATGCGCTGCACCGAGTGCGGTGTCAAGTGCCACGAGAAGTGCCAGGACCTGCTCAACGCCGACTGCCT
GCAGCGGGCTGCGGAGAAGAGCTCCAAGCACGGGGCGGAGGACCGGACACAGAACATCATCATGGTGCTC
AAGGACCGCATGAAGATCCGGGAGCGCAACAAGCCCGAGATCTTCGAGCTCATCCAGGAGATCTTCGCGG
TGACCAAGACGGCGCACACGCAGCAGATGAAGGCGGTCAAGCAGAGCGTGCTGGACGGCACGTCCAAGTG
GTCCGCCAAGATCAGCATCACCGTGGTCTGCGCCCAGGGCTTGCAGGCAAAGGACAAGACAGGATCCAGT
GACCCCTATGTCACCGTCCAGGTCGGGAAGACCAAGAAACGGACAAAAACCATCTATGGGAACCTCAACC
CGGTGTGGGAGGAGAATTTCCACTTTGAATGTCACAATTCCTCCGACCGCATCAAGGTGCGCGTCTGGGA
CGAGGATGACGACATCAAATCCCGCGTGAAACAGAGGTTCAAGAGGGAATCTGACGATTTCCTGGGGCAG
ACGATCATTGAGGTGCGGACGCTCAGCGGCGAGATGGACGTGTGGTACAACCTGGACAAGCGAACTGACA
AATCTGCCGTGTCGGGTGCCATCCGGCTCCACATCAGTGTGGAGATCAAAGGCGAGGAGAAGGTGGCCCC
GTACCATGTCCAGTACACCTGTCTGCATGAGAACCTGTTCCACTTCGTGACCGACGTGCAGAACAATGGG
GTCGTGAAGATCCCAGATGCCAAGGGTGACGATGCCTGGAAGGTTTACTACGATGAGACAGCCCAGGAGA
TTGTGGACGAGTTTGCCATGCGCTACGGCGTCGAGTCCATCTACCAAGCCATGACCCACTTTGCCTGCCT
CTCCTCCAAGTATATGTGCCCAGGGGTGCCTGCCGTCATGAGCACCCTGCTCGCCAACATCAATGCCTAC
TACGCACACACCACCGCCTCCACCAACGTGTCTGCCTCCGACCGCTTCGCCGCCTCCAACTTTGGGAAAG
AGCGCTTCGTGAAACTCCTGGACCAGCTGCATAACTCCCTGCGGATTGACCTCTCCATGTACCGGAATAA
CTTCCCAGCCAGCAGCCCGGAGAGACTCCAGGACCTCAAATCCACTGTGGACCTTCTCACCAGCATCACC
TTCTTTCGGATGAAGGTACAAGAACTCCAGAGCCCGCCCCGAGCCAGCCAGGTGGTAAAGGACTGTGTGA
AAGCCTGCCTTAATTCTACCTACGAGTACATCTTCAATAACTGCCATGAACTGTACAGCCGGGAGTACCA
GACAGACCCGGCCAAGAAGGGGGAAGTTCTCCCAGAGGAACAGGGGCCCAGCATCAAGAACCTCGACTTC
TGGTCCAAGCTGATTACCCTCATAGTGTCCATCATTGAGGAAGACAAGAATTCCTACACTCCCTGCCTCA
ACCAGTTTCCCCAGGAGCTGAATGTGGGTAAAATCAGCGCTGAAGTGATGTGGAATCTGTTTGCCCAAGA
CATGAAGTACGCCATGGAGGAGCACGACAAGCATCGTCTATGCAAGAGTGCCGACTACATGAACCTCCAC
TTCAAGGTGAAATGGCTCTACAATGAGTATGTGACGGAACTTCCCGCCTTCAAGGACCGCGTGCCTGAGT
ACCCTGCATGGTTTGAACCCTTCGTCATCCAGTGGCTGGATGAGAATGAGGAGGTGTCCCGGGATTTCCT
GCACGGTGCCCTGGAGCGAGACAAGAAGGATGGGTTCCAGCAGACCTCAGAGCATGCCCTATTCTCCTGC
TCCGTGGTGGATGTTTTCTCCCAACTCAACCAGAGCTTTGAAATCATCAAGAAACTCGAGTGTCCCGACC
CTCAGATCGTGGGGCACTACATGAGGCGCTTTGCCAAGACCATCAGTAATGTGCTCCTCCAGTATGCAGA
CATCATCTCCAAGGACTTTGCCTCCTACTGCTCCAAGGAGAAGGAGAAAGTGCCCTGCATTCTCATGAAT
AACACTCAACAGCTACGAGTTCAGCTGGAGAAGATGTTCGAAGCCATGGGAGGAAAGGAGCTGGATGCTG
AAGCCAGTGACATCCTGAAGGAGCTTCAGGTGAAACTCAATAACGTCTTGGATGAGCTCAGCCGGGTGTT
TGCTACCAGCTTCCAGCCGCACATTGAAGAGTGTGTCAAACAGATGGGTGACATCCTTAGCCAGGTTAAG
GGCACAGGCAATGTGCCAGCCAGTGCCTGCAGCAGCGTGGCCCAGGACGCGGACAATGTGTTGCAGCCCA
TCATGGACCTGCTGGACAGCAACCTGACCCTCTTTGCCAAAATCTGTGAGAAGACTGTGCTGAAGCGAGT
GCTGAAGGAGCTGTGGAAGCTGGTTATGAACACCATGGAGAAAACCATCGTCCTGCCGCCCCTCACTGAC
CAGACGGGAACCCAGATGATCTTCAATGCAGCCAAGGAGCTGGGTCAGCTGTCCAAACTCAAGGATCACA
TGGTACGAGAAGAAGCCAAGAGCTTGACCCCAAAGCAGTGCGCGGTTGTTGAGTTGGCCCTGGACACCAT
CAAGCAATATTTCCACGCGGGTGGCGTGGGCCTCAAGAAGACCTTCCTGGAGAAGAGCCCGGACCTGCAA
TCCTTGCGCTATGCCCTGTCGCTCTACACGCAGGCCACCGACCTGCTAATCAAGACCTTTGTACAGACGC
AATCGGCCCAGGGCTTGGGTGTAGAAGACCCTGTGGGTGAAGTCTCTGTCCATGTTGAGCTGTTCACTCA
TCCAGGAACTGGGGAACACAAGGTCACAGTGAAAGTGGTGGCTGCCAATGACCTCAAGTGGCAGACTTCT
GGCATCTTCCGGCCGTTCATCGAGGTCAACATCATTGGGCCCCAGCTCAGCGACAAGAAACGCAAGTTTG
CGACCAAATCCAAGAACAATAGCTGGGCTCCCAAGTACAATGAGAGCTTCCAGTTCACGCTGAGCGCCGA
CGCGGGTCCCGAGTGCTATGAGCTGCAGGTGTGCGTCAAGGACTACTGCTTCGCGCGCGAGGACCGCACG
GTGGGGCTGGCCGTGCTGCAGCTGCGTGAGCTGGCCCAGCGCGGGAGCGCCGCCTGCTGGCTGCCGCTCG
GCCGCCGCATCCACATGGACGACACGGGCCTCACGGTGCTGCGAATCCTCTCGCAGCGCAGCAACGACGA
GGTGGCCAAGGAGTTCGTGAAGCTCAAGTCGGACACGCGCTCCGCCGAGGAGGGCGGTGCCGCGCCTGCG
CCTTAGCGCGGGCGGTCGGCCGAGCGGCACTGCGCCTGCGCGGAGGGCGCTGGGCGGGGAGGGACGGGGC
TTGCGCCTTGGTGGGACCTCCCCAGGGGCGGGGCTCGGGGGGCTCCACGCCAAGGGTGGGCTGCGCCTAC
GCCCTTGACTCAGCTTTCCCTTTTGGGGAATTAGGAATGGAGGATGCCCCGCCCTCTCGGGAGGCCACGC
CCAAGGGCGCGACGAAGGAAGGAGCCACATCCCCAACTTGAGGCCACGCCCCCAGCACCTAGGGGGCATT
TTGAGCTGGGATGGGGGAAACCTCGTCCCTATGGAGGAGGCCACATCCCGGGGCTCTGGTACCGGGAGGC
ACCACCTCATGTCCCCTGGAAAAGCCATAAGATGGGACCCAGACCCCTGGGACCCCAGACCAATTGCCAA
GTATGGAAATCTCAGCTCCCTCGAGGGGGGGCCCTGGGCAAGGGGTAGGGCTCTCTGGAGCGCCCCTCTA
GGTGGCCTGGGGACTGGAGGGACCAGGATGCTGGTTGGAGGGCCCCGGAATACCGGAGTCCCTTTAGATA
TTTGTGCAAAAAATAAATGGGGGGAGGGGGGAGGATGGGATTTCAAAAGCACATGCGCCCTTGGGCGCCC
AAACCCTGGGGGCCGAGGGGACGGCTCTGGTTCCCCACGCTGCCCCTACTTCCCTTTGGGAGTTTGCCTC
TCCCTCTCCCCCAACAAACCCAGTCCTCATATCATAGAGTTCAACACACCCATTTGACAGATGGCAAAAC
TGAGGCTTAAAGAGCTGCTTGAGACTTGGCCAAGGTTCCAGGTGCCATACCCTCTGTGCCCCTCCCTTAG
GCCTGTGTGCCCCATGGAAGGGTGGGCTGAGATCGGGATGACCTGACACAGCTCCCTATTGCTGCTAATT
CCCCCTCGGCCTCCTCCAAGGGGTGGGAATTCCAGGCCAAGACCCCTACTTCGCCTTTCCTTCTCCGGCT
GCCAAGCAGGACCTTTGCCCTCAGCCCTTTCTCCTGGGATCTCCATGGGGGATGCCATGAGGGCCTCCCA
CCACAAAAGAGAATTTGGGATCCCCTGGTCCCAGGTTTCTCCATCCCTTCTTCCTTTTCCAGAATTTTCC
AAATAGGAAAGAACAGAAGGAGACCAGAAACTCTAGGGGGGAGAAAGAGAATGAGAGAAAGAGAATGAGA
GAGAGAGAAACACAAACACAGTGACACAGTGAGAGCTTAGTCTCCAAGAGCCTATTCATTGATTCAAACA
CCCAAGCCACAGGATACCTCAGATGGCCCTCTTGCCAGCTGGAAGCTCTTTCTCCAATGAGCAAAGTTAC
AGTGACCTGGCTGGAGTTACCTGGTGCACATAGGACCTTAGGGGAAAGTTCAGCGTGGACTACACTTGCT
CTGGGATCTGCTTTTCCACATGTGTGTATGGCACGCCTTTTTCTGCTGGATTGGGAAGGACAAGATTTTG
CTGTGCTAGGGAGAAATGAAAACGGGGTGAGCTGAGTAGCTGGGTTTCTGGAGGATAGAACATCAGATGG
GGAGGCTTTCCGAGGTGAAGAATGAGAGGGAACCACTTACTAGAGAGAAAAGAGCTCCAGGCCTGGGGAA
CAGCACGTGCGAAGGCCAGGAGAGAAGAACTGTTGAAACAACGAGAAGGGTGGCACGGCTGGAGCTGAGC
CAGCAAGGGGGATCGTGAGGAGCCTTGGGGTTGGGGAGATCTGCAGAAGCATCAGACCAGGCAGGGCCTC
GTACGCAGTCCTGAGGAGTTTTACTTTTATTCTAAGACAGTTGGGGAGCTCCAGGAGCTGTTTTAAGTTG
GGGAGAGACTGGATTCCAGCCTGCAAAAGCTGTTTTGTGAAGACTAAAACCAGTGAGGAGAGGTGGAGGT
GCTTTGGGGACACTGAAATGGATTCTTGGAAAGATTCTGAAGGCTGTGTTGAAAAGACACCTATAGCTGT
GGGGACATGACTATAATCCCAGCATTTGGGGAGACCGAGGCTGGCAGATCACTTAAGGTCAGGAGTTTGA
GACCAGCCTGGCCAACATGGCGAAACCCCATCTCTGCTAAAAATACAAAAATTAGCTGGGTGCAGTGGTG
CATGCCTGTAGTCCCAGCTACTCAGGAGACTGAGGCGGGAGAATTGTTTGAACCCTGGAGGCAGAGGTTG
TAGTGAGTCGTGATCACACAACTGCACTCCAGCCTGGGCAACAGAACAATACTCCATTCCCTCCCCTCTA
CCCCACCAAAAAAAAAAAAAAATCCTGCCCTTAGATGAGCTCTAGGGCTGCTGAGTACAGTTGTCCCAGT
TGCACAGTGCCCAAGGGTTTGGCATTGCTAAGAAGGCCACGTGCAAATCCTAGATATTGAGTGTTGTATG
TTTGTGACGTTGGTTTCCCGACATGTGAATGGCCCAAGTGTCTGGAAGAAGTGGCGCCACTTTCTAATTT
GCTTGGAGATGTTGCATGTCCCTTAAATTCAGACAGGTGCAGGTAACTGGAGGTTCTGAACCAAAGGTTA
AAATGCAAATTCTCATACAGGGTTGGGAAGTTGTAGCCAGGGATAAGCTTATGTGACTGTTATATGGACT
GAGGAGCAGATGTGAATTTCGAACCATGACATGGCTGAGGGTAGGGGTCGGGTGGATGGATGATTCAGGG
TTGTAACCCATAGAGCCCAAAGGGGAAGTGATCTGTGACCTGGGGTGAGGGTGATCTGGAAGATTTTTGG
ATGGCTGGAAAGAAATGGGGAAGTCGAGCTGCCTGAGAGAGCCAAGTTATTTCCCAAAAGATTCCTTAGG
AGTCTTTCTGTTCAAGACCTCCGTGTGTGTGTGTGTGTGTTTAGGGTTCCCCAGCAATGGCCCAGGCATG
TGAAGGAAACAAGCTTCTTCAGGGAATATTTGTTGAATGAGTTTTCCTGACTCCCAGGCTAGAACTGTTT
TTGCAATTTCCACCCTCTTTTCTTTCCCCCAGAGAACTCCTATTCGTCCTTCAAAACCCATCACGGAAAC
CCCTCTTGGAGAAAACCCTCCTTCCTTCCCCTCAGGACTTTCCCAGCCACCGTCTCTCCTCCAGTCCAGC
CTGATGCCATGGGACTGGGGGTTTCTCTGTCCAGCTCTGTTTCTCCCAGACTGGGGTCTGAGGACTCTCA
GGACCCCCAACTTTACCTAGCACAGGCTGGGCACAAGTGGGTGACAGGGAGTCTACGCCTAGTGGAATTA
TGTATTGGGGCAGGGTCAGTGTGAGAATACACATCCGCATGCATGTCTGTCCATGTCTGTCCGTACCAAC
CTTCCCCTTCCACACGGACCTGGGCACATAGGAGGTGTCTGAGCCTGACACATGGGACAGAGAGTGGACA
TGGCTGAGACACGGACAGAGAAAAGACAAGGAGTCCAGGGGGCTGAAAGCCTTTTGAAATCAGGAAGTTC
CTGTATTGGCAGAACAAAGCCCAGAGAGGAGCAGGGCTTTCCTCAACGCCACCCAGCAAGTGGACACAGA
GCCCGGCCTTGGATGACACCTCCAGGGTTCTGAACCCTGGACCTCGCTTTATGCAAGGAGCTGGCCCCAC
ATTTCCATGAATCGGGGAAACAGCACAAGAAGGTTGGCCTGTGGCAGGGCAAGGGTTAAAGGGGTGACAT
TGAGGGATGCCTCAGAGTCAAAGTCCCCTGACCAAGAGGAATAGAGTAGAAAACACAGAGACAGAGGGTG
AGATCACGCCCCGATGAGGACGGAGAGAGACAGAGATGGAGAGAGACATAGAGGTGGAAATATACAGAGA
AAGATAAATGCAGAGACCAAGGCAGGGAGTGTCGGGGGAAGTAAAGAGGGTGTCCTGAAGAAAGAAGGAT
CTGTTCACTCTTACCAGTCTGTCCTCGAATGATTTGCATAAAATGAGGAGGTGCCTGTCCACACCCCCAA
TTCCTCTCTCAGGCCCCAGAGCCTGAGACCTCACCATGCCCCCATCAGAGATGCAAAAAACTAAACACCC
AACTAGAAATCCTTGGGACCTCTCTCGGCTGGGATCTCAGAGCCTTTCTGTCCCCTACCCCTACCCCATG
TGCTGTCGATTTTGCAGATGGGGACAACCTGGGGCCTCCCGGAACTCTGCCACCCTGGGGAAGTTGGGGG
AGGGCCTTAGTCCCGGATCACAACCCCGTCTGCTCCCCAGAATCCTTTCCTAAGAATCGTTGAGGACCAA
AGTTGTCTTTGCTGACACGTGTTGCTTTTCTCTTTGCCTTTTATTGTTTCAGAGAAAAATCAAGTTGACT
GTGTCAAGTAACACCCCACCCCTTACCCCCGTCCAGCCATAGTGGCTCTCTGGAGACACAGGTCACAGGC
GGAGGGTCCCCTGATCATCCCCAACCACACAGCCAGGGGGACTTGACCCCTGTCCACCCCTGTCTCGTGC
TCCCTCAGACCCCCACAAACCGGCCAAGCAGTCCGGGGAGGCTTCCCCTCCACACAACTCTTAGCATGTG
ATTGCAGATGTGAAATCAAAACGTTGTTTGTTTTTTGTTTTGTTTTGATTCTACCCCGTCGGTCCAGTGT
CTGCACAGACGCCTTCATTTCTCTGTAAATATGTGACTTGGAACAAATGTTTAACACAAACGAGAAGTGG
TCATGAATGCATGGTGTTGAGATGTTTTGCACTATTCTGACTTTTTGGTCTCTGTAAAAATATTTTATTA
ACAGCAGACATTAAAAAAAGAAAAACCACACACA.

In various embodiments, an UNC13A mRNA transcript comprises the sequence provided as SEQ ID NO: 9591.

(SEQ ID NO: 9591 (SOURCE NCBI Reference Sequence XM_011527810.2))
CTGAACCAAGATGGCCGGTGGCGGCCGGGCCCCGGCGTGAGCCAAGCGCGGGCTGCAGCCGGGAGATGCC
CCAGCCCAGCGGCCGCTGAGCCCGACCCGACAGAGCCGGCCCGGCCGCCTCCGGCCCACCTGCGAGCTCG
GAGACATGTCTCTGCTTTGCGTTGGAGAGAAATTCAACACGTACGTGACCCTGAAAGTGCAGAATGTCAA
GAGCACGACCATCGCGGTGCGGGGCAGCCAGCCCAGCTGGGAGCAGGATTTCATGTTCGAGATTAACCGT
CTGGATTTGGGACTGACGGTGGAGGTGTGGAATAAGGGTCTCATCTGGGACACAATGGTGGGCACTGTGT
GGATCCCACTGAGGACCATCCGCCAGTCCAATGAGGAGGGCCCTGGAGAGTGGCTGACGCTGGACTCCCA
GGTCATCATGGCAGACAGTGAGATCTGTGGCACCAAGGACCCCACCTTCCACCGCATCCTCCTGGACACG
CGCTTTGAGCTACCCTTAGACATTCCTGAAGAGGAGGCTCGCTACTGGGCCAAGAAGCTGGAGCAGCTCA
ATGCTATGCGGGACCAGGATGAATATTCGTTCCAAGATGAGCAAGACAAGCCTCTGCCTGTCCCCAGCAA
CCAGTGCTGCAACTGGAATTATTTTGGCTGGGGTGAGCAGCACAACGATGACCCCGACAGTGCAGTGGAT
GATCGTGACAGTGACTACCGCAGTGAAACGAGCAACAGCATCCCGCCGCCCTATTATACTACGTCACAAC
CCAACGCCTCAGTCCACCAATATTCTGTTCGCCCACCACCCCTGGGCTCCCGGGAGTCCTACAGTGACTC
CATGCACAGTTACGAGGAGTTCTCTGAGCCACAAGCCCTCAGCCCCACGGGTAGCAGCCGCTATGCCTCT
TCCGGGGAGCTGAGCCAGGGAAGCTCTCAGCTGAGCGAGGACTTCGACCCTGACGAGCACAGCCTGCAGG
GCTCCGACATGGAGGATGAGCGGGACCGGGACTCCTACCACTCCTGCCACAGCTCGGTCAGCTACCACAA
AGACTCGCCTCGCTGGGACCAGGATGAGGAAGAGCTGGAGGAGGACCTGGAGGACTTCCTGGAGGAGGAG
GAGCTGCCTGAAGATGAGGAGGAGCTGGAGGAGGAGGAGGAGGAGGTGCCTGACGATTTGGGCAGCTATG
CCCAGCGTGAAGACGTAGCTGTGGCTGAGCCCAAAGACTTCAAACGCATCAGCCTCCCGCCAGCTGCCCC
AGGGAAGGAGGACAAGGCCCCAGTGGCACCCACCGAGGCCCCCGACATGGCCAAGGTGGCCCCCAAGCCA
GCCACGCCCGACAAGGTGCCTGCAGCTGAGCAGATCCCTGAGGCTGAGCCACCCAAGGACGAGGAGAGTT
TCAGGCCGAGAGAGGATGAGGAAGGCCAGGAGGGGCAGGACTCCATGTCCAGGGCCAAGGCCAACTGGCT
GCGTGCCTTCAACAAGGTGCGGATGCAGCTGCAGGAGGCCCGGGGAGAAGGAGAGATGTCTAAATCCCTA
TGGTTCAAAGGCGGCCCAGGGGGCGGTCTCATCATCATCGACAGCATGCCAGACATCCGCAAGAGGAAAC
CTATCCCACTCGTGAGCGACTTGGCCATGTCCCTGGTCCAGTCCAGGAAAGCGGGCATCACCTCGGCCTT
GGCCTCCAGCACGTTGAACAACGAGGAGCTGAAAAACCACGTTTACAAGAAGACCCTGCAAGCCTTAATC
TACCCCATCTCGTGCACGACGCCACACAACTTCGAAGTGTGGACGGCCACCACGCCCACCTACTGCTACG
AGTGCGAGGGGCTGCTGTGGGGCATCGCGAGGCAGGGCATGCGCTGCACCGAGTGCGGTGTCAAGTGCCA
CGAGAAGTGCCAGGACCTGCTCAACGCCGACTGCCTGCAGCGGGCTGCGGAGAAGAGCTCCAAGCACGGG
GCGGAGGACCGGACACAGAACATCATCATGGTGCTCAAGGACCGCATGAAGATCCGGGAGCGCAACAAGC
CCGAGATCTTCGAGCTCATCCAGGAGATCTTCGCGGTGACCAAGACGGCGCACACGCAGCAGATGAAGGC
GGTCAAGCAGAGCGTGCTGGACGGCACGTCCAAGTGGTCCGCCAAGATCAGCATCACCGTGGTCTGCGCC
CAGGGCTTGCAGGCAAAGGACAAGACAGGATCCAGTGACCCCTATGTCACCGTCCAGGTCGGGAAGACCA
AGAAACGGACAAAAACCATCTATGGGAACCTCAACCCGGTGTGGGAGGAGAATTTCCACTTTGAATGTCA
CAATTCCTCCGACCGCATCAAGGTGCGCGTCTGGGACGAGGATGACGACATCAAATCCCGCGTGAAACAG
AGGTTCAAGAGGGAATCTGACGATTTCCTGGGGCAGACGATCATTGAGGTGCGGACGCTCAGCGGCGAGA
TGGACGTGTGGTACAACCTGGACAAGCGAACTGACAAATCTGCCGTGTCGGGTGCCATCCGGCTCCACAT
CAGTGTGGAGATCAAAGGCGAGGAGAAGGTGGCCCCGTACCATGTCCAGTACACCTGTCTGCATGAGAAC
CTGTTCCACTTCGTGACCGACGTGCAGAACAATGGGGTCGTGAAGATCCCAGATGCCAAGGGTGACGATG
CCTGGAAGGTTTACTACGATGAGACAGCCCAGGAGATTGTGGACGAGTTTGCCATGCGCTACGGCGTCGA
GTCCATCTACCAAGCCATGACCCACTTTGCCTGCCTCTCCTCCAAGTATATGTGCCCAGGGGTGCCTGCC
GTCATGAGCACCCTGCTCGCCAACATCAATGCCTACTACGCACACACCACCGCCTCCACCAACGTGTCTG
CCTCCGACCGCTTCGCCGCCTCCAACTTTGGGAAAGAGCGCTTCGTGAAACTCCTGGACCAGCTGCATAA
CTCCCTGCGGATTGACCTCTCCATGTACCGGAATAACTTCCCAGCCAGCAGCCCGGAGAGACTCCAGGAC
CTCAAATCCACTGTGGACCTTCTCACCAGCATCACCTTCTTTCGGATGAAGGTACAAGAACTCCAGAGCC
CGCCCCGAGCCAGCCAGGTGGTAAAGGACTGTGTGAAAGCCTGCCTTAATTCTACCTACGAGTACATCTT
CAATAACTGCCATGAACTGTACAGCCGGGAGTACCAGACAGACCCGGCCAAGAAGGGGGAAGTTCTCCCA
GAGGAACAGGGGCCCAGCATCAAGAACCTCGACTTCTGGTCCAAGCTGATTACCCTCATAGTGTCCATCA
TTGAGGAAGACAAGAATTCCTACACTCCCTGCCTCAACCAGTTTCCCCAGGAGCTGAATGTGGGTAAAAT
CAGCGCTGAAGTGATGTGGAATCTGTTTGCCCAAGACATGAAGTACGCCATGGAGGAGCACGACAAGCAT
CGTCTATGCAAGAGTGCCGACTACATGAACCTCCACTTCAAGGTGAAATGGCTCTACAATGAGTATGTGA
CGGAACTTCCCGCCTTCAAGGACCGCGTGCCTGAGTACCCTGCATGGTTTGAACCCTTCGTCATCCAGTG
GCTGGATGAGAATGAGGAGGTGTCCCGGGATTTCCTGCACGGTGCCCTGGAGCGAGACAAGAAGGATGGG
TTCCAGCAGACCTCAGAGCATGCCCTATTCTCCTGCTCCGTGGTGGATGTTTTCTCCCAACTCAACCAGA
GCTTTGAAATCATCAAGAAACTCGAGTGTCCCGACCCTCAGATCGTGGGGCACTACATGAGGCGCTTTGC
CAAGACCATCAGTAATGTGCTCCTCCAGTATGCAGACATCATCTCCAAGGACTTTGCCTCCTACTGCTCC
AAGGAGAAGGAGAAAGTGCCCTGCATTCTCATGAATAACACTCAACAGCTACGAGTTCAGCTGGAGAAGA
TGTTCGAAGCCATGGGAGGAAAGGAGCTGGATGCTGAAGCCAGTGACATCCTGAAGGAGCTTCAGGTGAA
ACTCAATAACGTCTTGGATGAGCTCAGCCGGGTGTTTGCTACCAGCTTCCAGCCGCACATTGAAGAGTGT
GTCAAACAGATGGGTGACATCCTTAGCCAGGTTAAGGGCACAGGCAATGTGCCAGCCAGTGCCTGCAGCA
GCGTGGCCCAGGACGCGGACAATGTGTTGCAGCCCATCATGGACCTGCTGGACAGCAACCTGACCCTCTT
TGCCAAAATCTGTGAGAAGACTGTGCTGAAGCGAGTGCTGAAGGAGCTGTGGAAGCTGGTTATGAACACC
ATGGAGAAAACCATCGTCCTGCCGCCCCTCACTGACCAGACGATGATCGGGAACCTCTTGAGAAAACATG
GCAAGGGATTAGAAAAGGGCAGGGTGAAATTGCCAAGCCACTCAGACGGAACCCAGATGATCTTCAATGC
AGCCAAGGAGCTGGGTCAGCTGTCCAAACTCAAGGATCACATGGTACGAGAAGAAGCCAAGAGCTTGACC
CCAAAGCAGTGCGCGGTTGTTGAGTTGGCCCTGGACACCATCAAGCAATATTTCCACGCGGGTGGCGTGG
GCCTCAAGAAGACCTTCCTGGAGAAGAGCCCGGACCTGCAATCCTTGCGCTATGCCCTGTCGCTCTACAC
GCAGGCCACCGACCTGCTAATCAAGACCTTTGTACAGACGCAATCGGCCCAGGGCTTGGGTGTAGAAGAC
CCTGTGGGTGAAGTCTCTGTCCATGTTGAGCTGTTCACTCATCCAGGAACTGGGGAACACAAGGTCACAG
TGAAAGTGGTGGCTGCCAATGACCTCAAGTGGCAGACTTCTGGCATCTTCCGGCCGTTCATCGAGGTCAA
CATCATTGGGCCCCAGCTCAGCGACAAGAAACGCAAGTTTGCGACCAAATCCAAGAACAATAGCTGGGCT
CCCAAGTACAATGAGAGCTTCCAGTTCACGCTGAGCGCCGACGCGGGTCCCGAGTGCTATGAGCTGCAGG
TGTGCGTCAAGGACTACTGCTTCGCGCGCGAGGACCGCACGGTGGGGCTGGCCGTGCTGCAGCTGCGTGA
GCTGGCCCAGCGCGGGAGCGCCGCCTGCTGGCTGCCGCTCGGCCGCCGCATCCACATGGACGACACGGGC
CTCACGGTGCTGCGAATCCTCTCGCAGCGCAGCAACGACGAGGTGGCCAAGGAGTTCGTGAAGCTCAAGT
CGGACACGCGCTCCGCCGAGGAGGGCGGTGCCGCGCCTGCGCCTTAGCGCGGGCGGTCGGCCGAGCGGCA
CTGCGCCTGCGCGGAGGGCGCTGGGCGGGGAGGGACGGGGCTTGCGCCTTGGTGGGACCTCCCCAGGGGC
GGGGCTCGGGGGGCTCCACGCCAAGGGTGGGCTGCGCCTACGCCCTTGACTCAGCTTTCCCTTTTGGGGA
ATTAGGAATGGAGGATGCCCCGCCCTCTCGGGAGGCCACGCCCAAGGGCGCGACGAAGGAAGGAGCCACA
TCCCCAACTTGAGGCCACGCCCCCAGCACCTAGGGGGCATTTTGAGCTGGGATGGGGGAAACCTCGTCCC
TATGGAGGAGGCCACATCCCGGGGCTCTGGTACCGGGAGGCACCACCTCATGTCCCCTGGAAAAGCCATA
AGATGGGACCCAGACCCCTGGGACCCCAGACCAATTGCCAAGTATGGAAATCTCAGCTCCCTCGAGGGGG
GGCCCTGGGCAAGGGGTAGGGCTCTCTGGAGCGCCCCTCTAGGTGGCCTGGGGACTGGAGGGACCAGGAT
GCTGGTTGGAGGGCCCCGGAATACCGGAGTCCCTTTAGATATTTGTGCAAAAAATAAATGGGGGGAGGGG
GGAGGATGGGATTTCAAAAGCACATGCGCCCTTGGGCGCCCAAACCCTGGGGGCCGAGGGGACGGCTCTG
GTTCCCCACGCTGCCCCTACTTCCCTTTGGGAGTTTGCCTCTCCCTCTCCCCCAACAAACCCAGTCCTCA
TATCATAGAGTTCAACACACCCATTTGACAGATGGCAAAACTGAGGCTTAAAGAGCTGCTTGAGACTTGG
CCAAGGTTCCAGGTGCCATACCCTCTGTGCCCCTCCCTTAGGCCTGTGTGCCCCATGGAAGGGTGGGCTG
AGATCGGGATGACCTGACACAGCTCCCTATTGCTGCTAATTCCCCCTCGGCCTCCTCCAAGGGGTGGGAA
TTCCAGGCCAAGACCCCTACTTCGCCTTTCCTTCTCCGGCTGCCAAGCAGGACCTTTGCCCTCAGCCCTT
TCTCCTGGGATCTCCATGGGGGATGCCATGAGGGCCTCCCACCACAAAAGAGAATTTGGGATCCCCTGGT
CCCAGGTTTCTCCATCCCTTCTTCCTTTTCCAGAATTTTCCAAATAGGAAAGAACAGAAGGAGACCAGAA
ACTCTAGGGGGGAGAAAGAGAATGAGAGAAAGAGAATGAGAGAGAGAGAAACACAAACACAGTGACACAG
TGAGAGCTTAGTCTCCAAGAGCCTATTCATTGATTCAAACACCCAAGCCACAGGATACCTCAGATGGCCC
TCTTGCCAGCTGGAAGCTCTTTCTCCAATGAGCAAAGTTACAGTGACCTGGCTGGAGTTACCTGGTGCAC
ATAGGACCTTAGGGGAAAGTTCAGCGTGGACTACACTTGCTCTGGGATCTGCTTTTCCACATGTGTGTAT
GGCACGCCTTTTTCTGCTGGATTGGGAAGGACAAGATTTTGCTGTGCTAGGGAGAAATGAAAACGGGGTG
AGCTGAGTAGCTGGGTTTCTGGAGGATAGAACATCAGATGGGGAGGCTTTCCGAGGTGAAGAATGAGAGG
GAACCACTTACTAGAGAGAAAAGAGCTCCAGGCCTGGGGAACAGCACGTGCGAAGGCCAGGAGAGAAGAA
CTGTTGAAACAACGAGAAGGGTGGCACGGCTGGAGCTGAGCCAGCAAGGGGGATCGTGAGGAGCCTTGGG
GTTGGGGAGATCTGCAGAAGCATCAGACCAGGCAGGGCCTCGTACGCAGTCCTGAGGAGTTTTACTTTTA
TTCTAAGACAGTTGGGGAGCTCCAGGAGCTGTTTTAAGTTGGGGAGAGACTGGATTCCAGCCTGCAAAAG
CTGTTTTGTGAAGACTAAAACCAGTGAGGAGAGGTGGAGGTGCTTTGGGGACACTGAAATGGATTCTTGG
AAAGATTCTGAAGGCTGTGTTGAAAAGACACCTATAGCTGTGGGGACATGACTATAATCCCAGCATTTGG
GGAGACCGAGGCTGGCAGATCACTTAAGGTCAGGAGTTTGAGACCAGCCTGGCCAACATGGCGAAACCCC
ATCTCTGCTAAAAATACAAAAATTAGCTGGGTGCAGTGGTGCATGCCTGTAGTCCCAGCTACTCAGGAGA
CTGAGGCGGGAGAATTGTTTGAACCCTGGAGGCAGAGGTTGTAGTGAGTCGTGATCACACAACTGCACTC
CAGCCTGGGCAACAGAACAATACTCCATTCCCTCCCCTCTACCCCACCAAAAAAAAAAAAAAATCCTGCC
CTTAGATGAGCTCTAGGGCTGCTGAGTACAGTTGTCCCAGTTGCACAGTGCCCAAGGGTTTGGCATTGCT
AAGAAGGCCACGTGCAAATCCTAGATATTGAGTGTTGTATGTTTGTGACGTTGGTTTCCCGACATGTGAA
TGGCCCAAGTGTCTGGAAGAAGTGGCGCCACTTTCTAATTTGCTTGGAGATGTTGCATGTCCCTTAAATT
CAGACAGGTGCAGGTAACTGGAGGTTCTGAACCAAAGGTTAAAATGCAAATTCTCATACAGGGTTGGGAA
GTTGTAGCCAGGGATAAGCTTATGTGACTGTTATATGGACTGAGGAGCAGATGTGAATTTCGAACCATGA
CATGGCTGAGGGTAGGGGTCGGGTGGATGGATGATTCAGGGTTGTAACCCATAGAGCCCAAAGGGGAAGT
GATCTGTGACCTGGGGTGAGGGTGATCTGGAAGATTTTTGGATGGCTGGAAAGAAATGGGGAAGTCGAGC
TGCCTGAGAGAGCCAAGTTATTTCCCAAAAGATTCCTTAGGAGTCTTTCTGTTCAAGACCTCCGTGTGTG
TGTGTGTGTGTTTAGGGTTCCCCAGCAATGGCCCAGGCATGTGAAGGAAACAAGCTTCTTCAGGGAATAT
TTGTTGAATGAGTTTTCCTGACTCCCAGGCTAGAACTGTTTTTGCAATTTCCACCCTCTTTTCTTTCCCC
CAGAGAACTCCTATTCGTCCTTCAAAACCCATCACGGAAACCCCTCTTGGAGAAAACCCTCCTTCCTTCC
CCTCAGGACTTTCCCAGCCACCGTCTCTCCTCCAGTCCAGCCTGATGCCATGGGACTGGGGGTTTCTCTG
TCCAGCTCTGTTTCTCCCAGACTGGGGTCTGAGGACTCTCAGGACCCCCAACTTTACCTAGCACAGGCTG
GGCACAAGTGGGTGACAGGGAGTCTACGCCTAGTGGAATTATGTATTGGGGCAGGGTCAGTGTGAGAATA
CACATCCGCATGCATGTCTGTCCATGTCTGTCCGTACCAACCTTCCCCTTCCACACGGACCTGGGCACAT
AGGAGGTGTCTGAGCCTGACACATGGGACAGAGAGTGGACATGGCTGAGACACGGACAGAGAAAAGACAA
GGAGTCCAGGGGGCTGAAAGCCTTTTGAAATCAGGAAGTTCCTGTATTGGCAGAACAAAGCCCAGAGAGG
AGCAGGGCTTTCCTCAACGCCACCCAGCAAGTGGACACAGAGCCCGGCCTTGGATGACACCTCCAGGGTT
CTGAACCCTGGACCTCGCTTTATGCAAGGAGCTGGCCCCACATTTCCATGAATCGGGGAAACAGCACAAG
AAGGTTGGCCTGTGGCAGGGCAAGGGTTAAAGGGGTGACATTGAGGGATGCCTCAGAGTCAAAGTCCCCT
GACCAAGAGGAATAGAGTAGAAAACACAGAGACAGAGGGTGAGATCACGCCCCGATGAGGACGGAGAGAG
ACAGAGATGGAGAGAGACATAGAGGTGGAAATATACAGAGAAAGATAAATGCAGAGACCAAGGCAGGGAG
TGTCGGGGGAAGTAAAGAGGGTGTCCTGAAGAAAGAAGGATCTGTTCACTCTTACCAGTCTGTCCTCGAA
TGATTTGCATAAAATGAGGAGGTGCCTGTCCACACCCCCAATTCCTCTCTCAGGCCCCAGAGCCTGAGAC
CTCACCATGCCCCCATCAGAGATGCAAAAAACTAAACACCCAACTAGAAATCCTTGGGACCTCTCTCGGC
TGGGATCTCAGAGCCTTTCTGTCCCCTACCCCTACCCCATGTGCTGTCGATTTTGCAGATGGGGACAACC
TGGGGCCTCCCGGAACTCTGCCACCCTGGGGAAGTTGGGGGAGGGCCTTAGTCCCGGATCACAACCCCGT
CTGCTCCCCAGAATCCTTTCCTAAGAATCGTTGAGGACCAAAGTTGTCTTTGCTGACACGTGTTGCTTTT
CTCTTTGCCTTTTATTGTTTCAGAGAAAAATCAAGTTGACTGTGTCAAGTAACACCCCACCCCTTACCCC
CGTCCAGCCATAGTGGCTCTCTGGAGACACAGGTCACAGGCGGAGGGTCCCCTGATCATCCCCAACCACA
CAGCCAGGGGGACTTGACCCCTGTCCACCCCTGTCTCGTGCTCCCTCAGACCCCCACAAACCGGCCAAGC
AGTCCGGGGAGGCTTCCCCTCCACACAACTCTTAGCATGTGATTGCAGATGTGAAATCAAAACGTTGTTT
GTTTTTTGTTTTGTTTTGATTCTACCCCGTCGGTCCAGTGTCTGCACAGACGCCTTCATTTCTCTGTAAA
TATGTGACTTGGAACAAATGTTTAACACAAACGAGAAGTGGTCATGAATGCATGGTGTTGAGATGTTTTG
CACTATTCTGACTTTTTGGTCTCTGTAAAAATATTTTATTAACAGCAGACATTAAAAAAAGAAAAACCAC
ACACAGCCTTGGA.

In various embodiments, an UNC13A mRNA transcript comprises the sequence provided as SEQ ID NO: 9592.

(SEQ ID NO: 9592 (SOURCE (NCBI Reference Sequence XM_017026502.1))
CTGAACCAAGATGGCCGGTGGCGGCCGGGCCCCGGCGTGAGCCAAGCGCGGGCTGCAGCCGGGAGATGCC
CCAGCCCAGCGGCCGCTGAGCCCGACCCGACAGAGCCGGCCCGGCCGCCTCCGGCCCACCTGCGAGCTCG
GAGACATGTCTCTGCTTTGCGTTGGAGTCAAAAAAGCCAAGTTTGATGGTGCCCAAGAGAAATTCAACAC
GTACGTGACCCTGAAAGTGCAGAATGTCAAGAGCACGACCATCGCGGTGCGGGGCAGCCAGCCCAGCTGG
GAGCAGGATTTCATGTTCGAGATTAACCGTCTGGATTTGGGACTGACGGTGGAGGTGTGGAATAAGGGTC
TCATCTGGGACACAATGGTGGGCACTGTGTGGATCCCACTGAGGACCATCCGCCAGTCCAATGAGGAGGG
CCCTGGAGAGTGGCTGACGCTGGACTCCCAGGTCATCATGGCAGACAGTGAGATCTGTGGCACCAAGGAC
CCCACCTTCCACCGCATCCTCCTGGACACGCGCTTTGAGCTACCCTTAGACATTCCTGAAGAGGAGGCTC
GCTACTGGGCCAAGAAGCTGGAGCAGCTCAATGCTATGCGGGACCAGGATGAATATTCGTTCCAAGATGA
GCAAGACAAGCCTCTGCCTGTCCCCAGCAACCAGTGCTGCAACTGGAATTATTTTGGCTGGGGTGAGCAG
CACAACGATGACCCCGACAGTGCAGTGGATGATCGTGACAGTGACTACCGCAGTGAAACGAGCAACAGCA
TCCCGCCGCCCTATTATACTACGTCACAACCCAACGCCTCAGTCCACCAATATTCTGTTCGCCCACCACC
CCTGGGCTCCCGGGAGTCCTACAGTGACTCCATGCACAGTTACGAGGAGTTCTCTGAGCCACAAGCCCTC
AGCCCCACGGGTAGCAGCCGCTATGCCTCTTCCGGGGAGCTGAGCCAGGGAAGCTCTCAGCTGAGCGAGG
ACTTCGACCCTGACGAGCACAGCCTGCAGGGCTCCGACATGGAGGATGAGCGGGACCGGGACTCCTACCA
CTCCTGCCACAGCTCGGTCAGCTACCACAAAGACTCGCCTCGCTGGGACCAGGATGAGGAAGAGCTGGAG
GAGGACCTGGAGGACTTCCTGGAGGAGGAGGAGCTGCCTGAAGATGAGGAGGAGCTGGAGGAGGAGGAGG
AGGAGGTGCCTGACGATTTGGGCAGCTATGCCCAGCGTGAAGACGTAGCTGTGGCTGAGCCCAAAGACTT
CAAACGCATCAGCCTCCCGCCAGCTGCCCCAGGGAAGGAGGACAAGGCCCCAGTGGCACCCACCGAGGCC
CCCGACATGGCCAAGGTGGCCCCCAAGCCAGCCACGCCCGACAAGGTGCCTGCAGCTGAGCAGATCCCTG
AGGCTGAGCCACCCAAGGACGAGGAGAGTTTCAGGCCGAGAGAGGATGAGGAAGGCCAGGAGGGGCAGGA
CTCCATGTCCAGGGCCAAGGCCAACTGGCTGCGTGCCTTCAACAAGGTGCGGATGCAGCTGCAGGAGGCC
CGGGGAGAAGGAGAGATGTCTAAATCCCTATGGTTCAAAGGCGGCCCAGGGGGCGGTCTCATCATCATCG
ACAGCATGCCAGACATCCGCAAGAGGAAACCTATCCCACTCGTGAGCGACTTGGCCATGTCCCTGGTCCA
GTCCAGGAAAGCGGGCATCACCTCGGCCTTGGCCTCCAGCACGTTGAACAACGAGGAGCTGAAAAACCAC
GTTTACAAGAAGACCCTGCAAGCCTTAATCTACCCCATCTCGTGCACGACGCCACACAACTTCGAAGTGT
GGACGGCCACCACGCCCACCTACTGCTACGAGTGCGAGGGGCTGCTGTGGGGCATCGCGAGGCAGGGCAT
GCGCTGCACCGAGTGCGGTGTCAAGTGCCACGAGAAGTGCCAGGACCTGCTCAACGCCGACTGCCTGCAG
CGGGCTGCGGAGAAGAGCTCCAAGCACGGGGCGGAGGACCGGACACAGAACATCATCATGGTGCTCAAGG
ACCGCATGAAGATCCGGGAGCGCAACAAGCCCGAGATCTTCGAGCTCATCCAGGAGATCTTCGCGGTGAC
CAAGACGGCGCACACGCAGCAGATGAAGGCGGTCAAGCAGAGCGTGCTGGACGGCACGTCCAAGTGGTCC
GCCAAGATCAGCATCACCGTGGTCTGCGCCCAGGGCTTGCAGGCAAAGGACAAGACAGGATCCAGTGACC
CCTATGTCACCGTCCAGGTCGGGAAGACCAAGAAACGGACAAAAACCATCTATGGGAACCTCAACCCGGT
GTGGGAGGAGAATTTCCACTTTGAATGTCACAATTCCTCCGACCGCATCAAGGTGCGCGTCTGGGACGAG
GATGACGACATCAAATCCCGCGTGAAACAGAGGTTCAAGAGGGAATCTGACGATTTCCTGGGGCAGACGA
TCATTGAGGTGCGGACGCTCAGCGGCGAGATGGACGTGTGGTACAACCTGGACAAGCGAACTGACAAATC
TGCCGTGTCGGGTGCCATCCGGCTCCACATCAGTGTGGAGATCAAAGGCGAGGAGAAGGTGGCCCCGTAC
CATGTCCAGTACACCTGTCTGCATGAGAACCTGTTCCACTTCGTGACCGACGTGCAGAACAATGGGGTCG
TGAAGATCCCAGATGCCAAGGGTGACGATGCCTGGAAGGTTTACTACGATGAGACAGCCCAGGAGATTGT
GGACGAGTTTGCCATGCGCTACGGCGTCGAGTCCATCTACCAAGCCATGACCCACTTTGCCTGCCTCTCC
TCCAAGTATATGTGCCCAGGGGTGCCTGCCGTCATGAGCACCCTGCTCGCCAACATCAATGCCTACTACG
CACACACCACCGCCTCCACCAACGTGTCTGCCTCCGACCGCTTCGCCGCCTCCAACTTTGGGAAAGAGCG
CTTCGTGAAACTCCTGGACCAGCTGCATAACTCCCTGCGGATTGACCTCTCCATGTACCGGAATAACTTC
CCAGCCAGCAGCCCGGAGAGACTCCAGGACCTCAAATCCACTGTGGACCTTCTCACCAGCATCACCTTCT
TTCGGATGAAGGTACAAGAACTCCAGAGCCCGCCCCGAGCCAGCCAGGTGGTAAAGGACTGTGTGAAAGC
CTGCCTTAATTCTACCTACGAGTACATCTTCAATAACTGCCATGAACTGTACAGCCGGGAGTACCAGACA
GACCCGGCCAAGAAGGGGGAAGTTCTCCCAGAGGAACAGGGGCCCAGCATCAAGAACCTCGACTTCTGGT
CCAAGCTGATTACCCTCATAGTGTCCATCATTGAGGAAGACAAGAATTCCTACACTCCCTGCCTCAACCA
GTTTCCCCAGGAGCTGAATGTGGGTAAAATCAGCGCTGAAGTGATGTGGAATCTGTTTGCCCAAGACATG
AAGTACGCCATGGAGGAGCACGACAAGCATCGTCTATGCAAGAGTGCCGACTACATGAACCTCCACTTCA
AGGTGAAATGGCTCTACAATGAGTATGTGACGGAACTTCCCGCCTTCAAGGACCGCGTGCCTGAGTACCC
TGCATGGTTTGAACCCTTCGTCATCCAGTGGCTGGATGAGAATGAGGAGGTGTCCCGGGATTTCCTGCAC
GGTGCCCTGGAGCGAGACAAGAAGGATGGGTTCCAGCAGACCTCAGAGCATGCCCTATTCTCCTGCTCCG
TGGTGGATGTTTTCTCCCAACTCAACCAGAGCTTTGAAATCATCAAGAAACTCGAGTGTCCCGACCCTCA
GATCGTGGGGCACTACATGAGGCGCTTTGCCAAGACCATCAGTAATGTGCTCCTCCAGTATGCAGACATC
ATCTCCAAGGACTTTGCCTCCTACTGCTCCAAGGAGAAGGAGAAACCCTGCATTCTCATGAATAACACTC
AACAGCTACGAGTTCAGCTGGAGAAGATGTTCGAAGCCATGGGAGGAAAGGAGCTGGATGCTGAAGCCAG
TGACATCCTGAAGGAGCTTCAGGTGAAACTCAATAACGTCTTGGATGAGCTCAGCCGGGTGTTTGCTACC
AGCTTCCAGCCGCACATTGAAGAGTGTGTCAAACAGATGGGTGACATCCTTAGCCAGGTTAAGGGCACAG
GCAATGTGCCAGCCAGTGCCTGCAGCAGCGTGGCCCAGGACGCGGACAATGTGTTGCAGCCCATCATGGA
CCTGCTGGACAGCAACCTGACCCTCTTTGCCAAAATCTGTGAGAAGACTGTGCTGAAGCGAGTGCTGAAG
GAGCTGTGGAAGCTGGTTATGAACACCATGGAGAAAACCATCGTCCTGCCGCCCCTCACTGACCAGACGA
TGATCGGGAACCTCTTGAGAAAACATGGCAAGGGATTAGAAAAGGGCAGGGTGAAATTGCCAAGCCACTC
AGACGGAACCCAGATGATCTTCAATGCAGCCAAGGAGCTGGGTCAGCTGTCCAAACTCAAGGATCACATG
GTACGAGAAGAAGCCAAGAGCTTGACCCCAAAGCAGTGCGCGGTTGTTGAGTTGGCCCTGGACACCATCA
AGCAATATTTCCACGCGGGTGGCGTGGGCCTCAAGAAGACCTTCCTGGAGAAGAGCCCGGACCTGCAATC
CTTGCGCTATGCCCTGTCGCTCTACACGCAGGCCACCGACCTGCTAATCAAGACCTTTGTACAGACGCAA
TCGGCCCAGGGCTTGGGTGTAGAAGACCCTGTGGGTGAAGTCTCTGTCCATGTTGAGCTGTTCACTCATC
CAGGAACTGGGGAACACAAGGTCACAGTGAAAGTGGTGGCTGCCAATGACCTCAAGTGGCAGACTTCTGG
CATCTTCCGGCCGTTCATCGAGGTCAACATCATTGGGCCCCAGCTCAGCGACAAGAAACGCAAGTTTGCG
ACCAAATCCAAGAACAATAGCTGGGCTCCCAAGTACAATGAGAGCTTCCAGTTCACGCTGAGCGCCGACG
CGGGTCCCGAGTGCTATGAGCTGCAGGTGTGCGTCAAGGACTACTGCTTCGCGCGCGAGGACCGCACGGT
GGGGCTGGCCGTGCTGCAGCTGCGTGAGCTGGCCCAGCGCGGGAGCGCCGCCTGCTGGCTGCCGCTCGGC
CGCCGCATCCACATGGACGACACGGGCCTCACGGTGCTGCGAATCCTCTCGCAGCGCAGCAACGACGAGG
TGGCCAAGGAGTTCGTGAAGCTCAAGTCGGACACGCGCTCCGCCGAGGAGGGCGGTGCCGCGCCTGCGCC
TTAGCGCGGGCGGTCGGCCGAGCGGCACTGCGCCTGCGCGGAGGGCGCTGGGCGGGGAGGGACGGGGCTT
GCGCCTTGGTGGGACCTCCCCAGGGGCGGGGCTCGGGGGGCTCCACGCCAAGGGTGGGCTGCGCCTACGC
CCTTGACTCAGCTTTCCCTTTTGGGGAATTAGGAATGGAGGATGCCCCGCCCTCTCGGGAGGCCACGCCC
AAGGGCGCGACGAAGGAAGGAGCCACATCCCCAACTTGAGGCCACGCCCCCAGCACCTAGGGGGCATTTT
GAGCTGGGATGGGGGAAACCTCGTCCCTATGGAGGAGGCCACATCCCGGGGCTCTGGTACCGGGAGGCAC
CACCTCATGTCCCCTGGAAAAGCCATAAGATGGGACCCAGACCCCTGGGACCCCAGACCAATTGCCAAGT
ATGGAAATCTCAGCTCCCTCGAGGGGGGGCCCTGGGCAAGGGGTAGGGCTCTCTGGAGCGCCCCTCTAGG
TGGCCTGGGGACTGGAGGGACCAGGATGCTGGTTGGAGGGCCCCGGAATACCGGAGTCCCTTTAGATATT
TGTGCAAAAAATAAATGGGGGGAGGGGGGAGGATGGGATTTCAAAAGCACATGCGCCCTTGGGCGCCCAA
ACCCTGGGGGCCGAGGGGACGGCTCTGGTTCCCCACGCTGCCCCTACTTCCCTTTGGGAGTTTGCCTCTC
CCTCTCCCCCAACAAACCCAGTCCTCATATCATAGAGTTCAACACACCCATTTGACAGATGGCAAAACTG
AGGCTTAAAGAGCTGCTTGAGACTTGGCCAAGGTTCCAGGTGCCATACCCTCTGTGCCCCTCCCTTAGGC
CTGTGTGCCCCATGGAAGGGTGGGCTGAGATCGGGATGACCTGACACAGCTCCCTATTGCTGCTAATTCC
CCCTCGGCCTCCTCCAAGGGGTGGGAATTCCAGGCCAAGACCCCTACTTCGCCTTTCCTTCTCCGGCTGC
CAAGCAGGACCTTTGCCCTCAGCCCTTTCTCCTGGGATCTCCATGGGGGATGCCATGAGGGCCTCCCACC
ACAAAAGAGAATTTGGGATCCCCTGGTCCCAGGTTTCTCCATCCCTTCTTCCTTTTCCAGAATTTTCCAA
ATAGGAAAGAACAGAAGGAGACCAGAAACTCTAGGGGGGAGAAAGAGAATGAGAGAAAGAGAATGAGAGA
GAGAGAAACACAAACACAGTGACACAGTGAGAGCTTAGTCTCCAAGAGCCTATTCATTGATTCAAACACC
CAAGCCACAGGATACCTCAGATGGCCCTCTTGCCAGCTGGAAGCTCTTTCTCCAATGAGCAAAGTTACAG
TGACCTGGCTGGAGTTACCTGGTGCACATAGGACCTTAGGGGAAAGTTCAGCGTGGACTACACTTGCTCT
GGGATCTGCTTTTCCACATGTGTGTATGGCACGCCTTTTTCTGCTGGATTGGGAAGGACAAGATTTTGCT
GTGCTAGGGAGAAATGAAAACGGGGTGAGCTGAGTAGCTGGGTTTCTGGAGGATAGAACATCAGATGGGG
AGGCTTTCCGAGGTGAAGAATGAGAGGGAACCACTTACTAGAGAGAAAAGAGCTCCAGGCCTGGGGAACA
GCACGTGCGAAGGCCAGGAGAGAAGAACTGTTGAAACAACGAGAAGGGTGGCACGGCTGGAGCTGAGCCA
GCAAGGGGGATCGTGAGGAGCCTTGGGGTTGGGGAGATCTGCAGAAGCATCAGACCAGGCAGGGCCTCGT
ACGCAGTCCTGAGGAGTTTTACTTTTATTCTAAGACAGTTGGGGAGCTCCAGGAGCTGTTTTAAGTTGGG
GAGAGACTGGATTCCAGCCTGCAAAAGCTGTTTTGTGAAGACTAAAACCAGTGAGGAGAGGTGGAGGTGC
TTTGGGGACACTGAAATGGATTCTTGGAAAGATTCTGAAGGCTGTGTTGAAAAGACACCTATAGCTGTGG
GGACATGACTATAATCCCAGCATTTGGGGAGACCGAGGCTGGCAGATCACTTAAGGTCAGGAGTTTGAGA
CCAGCCTGGCCAACATGGCGAAACCCCATCTCTGCTAAAAATACAAAAATTAGCTGGGTGCAGTGGTGCA
TGCCTGTAGTCCCAGCTACTCAGGAGACTGAGGCGGGAGAATTGTTTGAACCCTGGAGGCAGAGGTTGTA
GTGAGTCGTGATCACACAACTGCACTCCAGCCTGGGCAACAGAACAATACTCCATTCCCTCCCCTCTACC
CCACCAAAAAAAAAAAAAAATCCTGCCCTTAGATGAGCTCTAGGGCTGCTGAGTACAGTTGTCCCAGTTG
CACAGTGCCCAAGGGTTTGGCATTGCTAAGAAGGCCACGTGCAAATCCTAGATATTGAGTGTTGTATGTT
TGTGACGTTGGTTTCCCGACATGTGAATGGCCCAAGTGTCTGGAAGAAGTGGCGCCACTTTCTAATTTGC
TTGGAGATGTTGCATGTCCCTTAAATTCAGACAGGTGCAGGTAACTGGAGGTTCTGAACCAAAGGTTAAA
ATGCAAATTCTCATACAGGGTTGGGAAGTTGTAGCCAGGGATAAGCTTATGTGACTGTTATATGGACTGA
GGAGCAGATGTGAATTTCGAACCATGACATGGCTGAGGGTAGGGGTCGGGTGGATGGATGATTCAGGGTT
GTAACCCATAGAGCCCAAAGGGGAAGTGATCTGTGACCTGGGGTGAGGGTGATCTGGAAGATTTTTGGAT
GGCTGGAAAGAAATGGGGAAGTCGAGCTGCCTGAGAGAGCCAAGTTATTTCCCAAAAGATTCCTTAGGAG
TCTTTCTGTTCAAGACCTCCGTGTGTGTGTGTGTGTGTTTAGGGTTCCCCAGCAATGGCCCAGGCATGTG
AAGGAAACAAGCTTCTTCAGGGAATATTTGTTGAATGAGTTTTCCTGACTCCCAGGCTAGAACTGTTTTT
GCAATTTCCACCCTCTTTTCTTTCCCCCAGAGAACTCCTATTCGTCCTTCAAAACCCATCACGGAAACCC
CTCTTGGAGAAAACCCTCCTTCCTTCCCCTCAGGACTTTCCCAGCCACCGTCTCTCCTCCAGTCCAGCCT
GATGCCATGGGACTGGGGGTTTCTCTGTCCAGCTCTGTTTCTCCCAGACTGGGGTCTGAGGACTCTCAGG
ACCCCCAACTTTACCTAGCACAGGCTGGGCACAAGTGGGTGACAGGGAGTCTACGCCTAGTGGAATTATG
TATTGGGGCAGGGTCAGTGTGAGAATACACATCCGCATGCATGTCTGTCCATGTCTGTCCGTACCAACCT
TCCCCTTCCACACGGACCTGGGCACATAGGAGGTGTCTGAGCCTGACACATGGGACAGAGAGTGGACATG
GCTGAGACACGGACAGAGAAAAGACAAGGAGTCCAGGGGGCTGAAAGCCTTTTGAAATCAGGAAGTTCCT
GTATTGGCAGAACAAAGCCCAGAGAGGAGCAGGGCTTTCCTCAACGCCACCCAGCAAGTGGACACAGAGC
CCGGCCTTGGATGACACCTCCAGGGTTCTGAACCCTGGACCTCGCTTTATGCAAGGAGCTGGCCCCACAT
TTCCATGAATCGGGGAAACAGCACAAGAAGGTTGGCCTGTGGCAGGGCAAGGGTTAAAGGGGTGACATTG
AGGGATGCCTCAGAGTCAAAGTCCCCTGACCAAGAGGAATAGAGTAGAAAACACAGAGACAGAGGGTGAG
ATCACGCCCCGATGAGGACGGAGAGAGACAGAGATGGAGAGAGACATAGAGGTGGAAATATACAGAGAAA
GATAAATGCAGAGACCAAGGCAGGGAGTGTCGGGGGAAGTAAAGAGGGTGTCCTGAAGAAAGAAGGATCT
GTTCACTCTTACCAGTCTGTCCTCGAATGATTTGCATAAAATGAGGAGGTGCCTGTCCACACCCCCAATT
CCTCTCTCAGGCCCCAGAGCCTGAGACCTCACCATGCCCCCATCAGAGATGCAAAAAACTAAACACCCAA
CTAGAAATCCTTGGGACCTCTCTCGGCTGGGATCTCAGAGCCTTTCTGTCCCCTACCCCTACCCCATGTG
CTGTCGATTTTGCAGATGGGGACAACCTGGGGCCTCCCGGAACTCTGCCACCCTGGGGAAGTTGGGGGAG
GGCCTTAGTCCCGGATCACAACCCCGTCTGCTCCCCAGAATCCTTTCCTAAGAATCGTTGAGGACCAAAG
TTGTCTTTGCTGACACGTGTTGCTTTTCTCTTTGCCTTTTATTGTTTCAGAGAAAAATCAAGTTGACTGT
GTCAAGTAACACCCCACCCCTTACCCCCGTCCAGCCATAGTGGCTCTCTGGAGACACAGGTCACAGGCGG
AGGGTCCCCTGATCATCCCCAACCACACAGCCAGGGGGACTTGACCCCTGTCCACCCCTGTCTCGTGCTC
CCTCAGACCCCCACAAACCGGCCAAGCAGTCCGGGGAGGCTTCCCCTCCACACAACTCTTAGCATGTGAT
TGCAGATGTGAAATCAAAACGTTGTTTGTTTTTTGTTTTGTTTTGATTCTACCCCGTCGGTCCAGTGTCT
GCACAGACGCCTTCATTTCTCTGTAAATATGTGACTTGGAACAAATGTTTAACACAAACGAGAAGTGGTC
ATGAATGCATGGTGTTGAGATGTTTTGCACTATTCTGACTTTTTGGTCTCTGTAAAAATATTTTATTAAC
AGCAGACATTAAAAAAAGAAAAACCACACACAGCCTTGGA.

In various embodiments, an UNC13A mRNA transcript comprises the sequence provided as SEQ ID NO: 9593.

(SEQ ID NO: 9593 (SOURCE NCBI Reference Sequence XM 011527811.2))
CTGAACCAAGATGGCCGGTGGCGGCCGGGCCCCGGCGTGAGCCAAGCGCGGGCTGCAGCCGGGAGATGCC
CCAGCCCAGCGGCCGCTGAGCCCGACCCGACAGAGCCGGCCCGGCCGCCTCCGGCCCACCTGCGAGCTCG
GAGACATGTCTCTGCTTTGCGTTGGAGTCAAAAAAGCCAAGTTTGATGGTGCCCAAGAGAAATTCAACAC
GTACGTGACCCTGAAAGTGCAGAATGTCAAGAGCACGACCATCGCGGTGCGGGGCAGCCAGCCCAGCTGG
GAGCAGGATTTCATGTTCGAGATTAACCGTCTGGATTTGGGACTGACGGTGGAGGTGTGGAATAAGGGTC
TCATCTGGGACACAATGGTGGGCACTGTGTGGATCCCACTGAGGACCATCCGCCAGTCCAATGAGGAGGG
CCCTGGAGAGTGGCTGACGCTGGACTCCCAGGTCATCATGGCAGACAGTGAGATCTGTGGCACCAAGGAC
CCCACCTTCCACCGCATCCTCCTGGACACGCGCTTTGAGCTACCCTTAGACATTCCTGAAGAGGAGGCTC
GCTACTGGGCCAAGAAGCTGGAGCAGCTCAATGCTATGCGGGACCAGGATGAATATTCGTTCCAAGATGA
GCAAGACAAGCCTCTGCCTGTCCCCAGCAACCAGTGCTGCAACTGGAATTATTTTGGCTGGGGTGAGCAG
CACAACGATGACCCCGACAGTGCAGTGGATGATCGTGACAGTGACTACCGCAGTGAAACGAGCAACAGCA
TCCCGCCGCCCTATTATACTACGTCACAACCCAACGCCTCAGTCCACCAATATTCTGTTCGCCCACCACC
CCTGGGCTCCCGGGAGTCCTACAGTGACTCCATGCACAGTTACGAGGAGTTCTCTGAGCCACAAGCCCTC
AGCCCCACGGGTAGCAGCCGCTATGCCTCTTCCGGGGAGCTGAGCCAGGGAAGCTCTCAGCTGAGCGAGG
ACTTCGACCCTGACGAGCACAGCCTGCAGGGCTCCGACATGGAGGATGAGCGGGACCGGGACTCCTACCA
CTCCTGCCACAGCTCGGTCAGCTACCACAAAGACTCGCCTCGCTGGGACCAGGATGAGGAAGAGCTGGAG
GAGGACCTGGAGGACTTCCTGGAGGAGGAGGAGCTGCCTGAAGATGAGGAGGAGCTGGAGGAGGAGGAGG
AGGAGGTGCCTGACGATTTGGGCAGCTATGCCCAGCGTGAAGACGTAGCTGTGGCTGAGCCCAAAGACTT
CAAACGCATCAGCCTCCCGCCAGCTGCCCCAGGGAAGGAGGACAAGGCCCCAGTGGCACCCACCGAGGCC
CCCGACATGGCCAAGGTGGCCCCCAAGCCAGCCACGCCCGACAAGGTGCCTGCAGCTGAGCAGATCCCTG
AGGCTGAGCCACCCAAGGACGAGGAGAGTTTCAGGCCGAGAGAGGATGAGGAAGGCCAGGAGGGGCAGGA
CTCCATGTCCAGGGCCAAGGCCAACTGGCTGCGTGCCTTCAACAAGGTGCGGATGCAGCTGCAGGAGGCC
CGGGGAGAAGGAGAGATGTCTAAATCCCTATGGTTCAAAGGCGGCCCAGGGGGCGGTCTCATCATCATCG
ACAGCATGCCAGACATCCGCAAGAGGAAACCTATCCCACTCGTGAGCGACTTGGCCATGTCCCTGGTCCA
GTCCAGGAAAGCGGGCATCACCTCGGCCTTGGCCTCCAGCACGTTGAACAACGAGGAGCTGAAAAACCAC
GTTTACAAGAAGACCCTGCAAGCCTTAATCTACCCCATCTCGTGCACGACGCCACACAACTTCGAAGTGT
GGACGGCCACCACGCCCACCTACTGCTACGAGTGCGAGGGGCTGCTGTGGGGCATCGCGAGGCAGGGCAT
GCGCTGCACCGAGTGCGGTGTCAAGTGCCACGAGAAGTGCCAGGACCTGCTCAACGCCGACTGCCTGCAG
CGGGCTGCGGAGAAGAGCTCCAAGCACGGGGCGGAGGACCGGACACAGAACATCATCATGGTGCTCAAGG
ACCGCATGAAGATCCGGGAGCGCAACAAGCCCGAGATCTTCGAGCTCATCCAGGAGATCTTCGCGGTGAC
CAAGACGGCGCACACGCAGCAGATGAAGGCGGTCAAGCAGAGCGTGCTGGACGGCACGTCCAAGTGGTCC
GCCAAGATCAGCATCACCGTGGTCTGCGCCCAGGGCTTGCAGGCAAAGGACAAGACAGGATCCAGTGACC
CCTATGTCACCGTCCAGGTCGGGAAGACCAAGAAACGGACAAAAACCATCTATGGGAACCTCAACCCGGT
GTGGGAGGAGAATTTCCACTTTGAATGTCACAATTCCTCCGACCGCATCAAGGTGCGCGTCTGGGACGAG
GATGACGACATCAAATCCCGCGTGAAACAGAGGTTCAAGAGGGAATCTGACGATTTCCTGGGGCAGACGA
TCATTGAGGTGCGGACGCTCAGCGGCGAGATGGACGTGTGGTACAACCTGGACAAGCGAACTGACAAATC
TGCCGTGTCGGGTGCCATCCGGCTCCACATCAGTGTGGAGATCAAAGGCGAGGAGAAGGTGGCCCCGTAC
CATGTCCAGTACACCTGTCTGCATGAGAACCTGTTCCACTTCGTGACCGACGTGCAGAACAATGGGGTCG
TGAAGATCCCAGATGCCAAGGGTGACGATGCCTGGAAGGTTTACTACGATGAGACAGCCCAGGAGATTGT
GGACGAGTTTGCCATGCGCTACGGCGTCGAGTCCATCTACCAAGCCATGACCCACTTTGCCTGCCTCTCC
TCCAAGTATATGTGCCCAGGGGTGCCTGCCGTCATGAGCACCCTGCTCGCCAACATCAATGCCTACTACG
CACACACCACCGCCTCCACCAACGTGTCTGCCTCCGACCGCTTCGCCGCCTCCAACTTTGGGAAAGAGCG
CTTCGTGAAACTCCTGGACCAGCTGCATAACTCCCTGCGGATTGACCTCTCCATGTACCGGAATAACTTC
CCAGCCAGCAGCCCGGAGAGACTCCAGGACCTCAAATCCACTGTGGACCTTCTCACCAGCATCACCTTCT
TTCGGATGAAGGTACAAGAACTCCAGAGCCCGCCCCGAGCCAGCCAGGTGGTAAAGGACTGTGTGAAAGC
CTGCCTTAATTCTACCTACGAGTACATCTTCAATAACTGCCATGAACTGTACAGCCGGGAGTACCAGACA
GACCCGGCCAAGAAGGGGGAAGTTCTCCCAGAGGAACAGGGGCCCAGCATCAAGAACCTCGACTTCTGGT
CCAAGCTGATTACCCTCATAGTGTCCATCATTGAGGAAGACAAGAATTCCTACACTCCCTGCCTCAACCA
GTTTCCCCAGGAGCTGAATGTGGGTAAAATCAGCGCTGAAGTGATGTGGAATCTGTTTGCCCAAGACATG
AAGTACGCCATGGAGGAGCACGACAAGCATCGTCTATGCAAGAGTGCCGACTACATGAACCTCCACTTCA
AGGTGAAATGGCTCTACAATGAGTATGTGACGGAACTTCCCGCCTTCAAGGACCGCGTGCCTGAGTACCC
TGCATGGTTTGAACCCTTCGTCATCCAGTGGCTGGATGAGAATGAGGAGGTGTCCCGGGATTTCCTGCAC
GGTGCCCTGGAGCGAGACAAGAAGGATGGGTTCCAGCAGACCTCAGAGCATGCCCTATTCTCCTGCTCCG
TGGTGGATGTTTTCTCCCAACTCAACCAGAGCTTTGAAATCATCAAGAAACTCGAGTGTCCCGACCCTCA
GATCGTGGGGCACTACATGAGGCGCTTTGCCAAGACCATCAGTAATGTGCTCCTCCAGTATGCAGACATC
ATCTCCAAGGACTTTGCCTCCTACTGCTCCAAGGAGAAGGAGAAAGTGCCCTGCATTCTCATGAATAACA
CTCAACAGCTACGAGTTCAGCTGGAGAAGATGTTCGAAGCCATGGGAGGAAAGGAGCTGGATGCTGAAGC
CAGTGACATCCTGAAGGAGCTTCAGGTGAAACTCAATAACGTCTTGGATGAGCTCAGCCGGGTGTTTGCT
ACCAGCTTCCAGCCGCACATTGAAGAGTGTGTCAAACAGATGGGTGACATCCTTAGCCAGGTTAAGGGCA
CAGGCAATGTGCCAGCCAGTGCCTGCAGCAGCGTGGCCCAGGACGCGGACAATGTGTTGCAGCCCATCAT
GGACCTGCTGGACAGCAACCTGACCCTCTTTGCCAAAATCTGTGAGAAGACTGTGCTGAAGCGAGTGCTG
AAGGAGCTGTGGAAGCTGGTTATGAACACCATGGAGAAAACCATCGTCCTGCCGCCCCTCACTGACCAGA
CGATGATCGGAACCCAGATGATCTTCAATGCAGCCAAGGAGCTGGGTCAGCTGTCCAAACTCAAGGATCA
CATGGTACGAGAAGAAGCCAAGAGCTTGACCCCAAAGCAGTGCGCGGTTGTTGAGTTGGCCCTGGACACC
ATCAAGCAATATTTCCACGCGGGTGGCGTGGGCCTCAAGAAGACCTTCCTGGAGAAGAGCCCGGACCTGC
AATCCTTGCGCTATGCCCTGTCGCTCTACACGCAGGCCACCGACCTGCTAATCAAGACCTTTGTACAGAC
GCAATCGGCCCAGGGCTTGGGTGTAGAAGACCCTGTGGGTGAAGTCTCTGTCCATGTTGAGCTGTTCACT
CATCCAGGAACTGGGGAACACAAGGTCACAGTGAAAGTGGTGGCTGCCAATGACCTCAAGTGGCAGACTT
CTGGCATCTTCCGGCCGTTCATCGAGGTCAACATCATTGGGCCCCAGCTCAGCGACAAGAAACGCAAGTT
TGCGACCAAATCCAAGAACAATAGCTGGGCTCCCAAGTACAATGAGAGCTTCCAGTTCACGCTGAGCGCC
GACGCGGGTCCCGAGTGCTATGAGCTGCAGGTGTGCGTCAAGGACTACTGCTTCGCGCGCGAGGACCGCA
CGGTGGGGCTGGCCGTGCTGCAGCTGCGTGAGCTGGCCCAGCGCGGGAGCGCCGCCTGCTGGCTGCCGCT
CGGCCGCCGCATCCACATGGACGACACGGGCCTCACGGTGCTGCGAATCCTCTCGCAGCGCAGCAACGAC
GAGGTGGCCAAGGAGTTCGTGAAGCTCAAGTCGGACACGCGCTCCGCCGAGGAGGGCGGTGCCGCGCCTG
CGCCTTAGCGCGGGCGGTCGGCCGAGCGGCACTGCGCCTGCGCGGAGGGCGCTGGGCGGGGAGGGACGGG
GCTTGCGCCTTGGTGGGACCTCCCCAGGGGCGGGGCTCGGGGGGCTCCACGCCAAGGGTGGGCTGCGCCT
ACGCCCTTGACTCAGCTTTCCCTTTTGGGGAATTAGGAATGGAGGATGCCCCGCCCTCTCGGGAGGCCAC
GCCCAAGGGCGCGACGAAGGAAGGAGCCACATCCCCAACTTGAGGCCACGCCCCCAGCACCTAGGGGGCA
TTTTGAGCTGGGATGGGGGAAACCTCGTCCCTATGGAGGAGGCCACATCCCGGGGCTCTGGTACCGGGAG
GCACCACCTCATGTCCCCTGGAAAAGCCATAAGATGGGACCCAGACCCCTGGGACCCCAGACCAATTGCC
AAGTATGGAAATCTCAGCTCCCTCGAGGGGGGGCCCTGGGCAAGGGGTAGGGCTCTCTGGAGCGCCCCTC
TAGGTGGCCTGGGGACTGGAGGGACCAGGATGCTGGTTGGAGGGCCCCGGAATACCGGAGTCCCTTTAGA
TATTTGTGCAAAAAATAAATGGGGGGAGGGGGGAGGATGGGATTTCAAAAGCACATGCGCCCTTGGGCGC
CCAAACCCTGGGGGCCGAGGGGACGGCTCTGGTTCCCCACGCTGCCCCTACTTCCCTTTGGGAGTTTGCC
TCTCCCTCTCCCCCAACAAACCCAGTCCTCATATCATAGAGTTCAACACACCCATTTGACAGATGGCAAA
ACTGAGGCTTAAAGAGCTGCTTGAGACTTGGCCAAGGTTCCAGGTGCCATACCCTCTGTGCCCCTCCCTT
AGGCCTGTGTGCCCCATGGAAGGGTGGGCTGAGATCGGGATGACCTGACACAGCTCCCTATTGCTGCTAA
TTCCCCCTCGGCCTCCTCCAAGGGGTGGGAATTCCAGGCCAAGACCCCTACTTCGCCTTTCCTTCTCCGG
CTGCCAAGCAGGACCTTTGCCCTCAGCCCTTTCTCCTGGGATCTCCATGGGGGATGCCATGAGGGCCTCC
CACCACAAAAGAGAATTTGGGATCCCCTGGTCCCAGGTTTCTCCATCCCTTCTTCCTTTTCCAGAATTTT
CCAAATAGGAAAGAACAGAAGGAGACCAGAAACTCTAGGGGGGAGAAAGAGAATGAGAGAAAGAGAATGA
GAGAGAGAGAAACACAAACACAGTGACACAGTGAGAGCTTAGTCTCCAAGAGCCTATTCATTGATTCAAA
CACCCAAGCCACAGGATACCTCAGATGGCCCTCTTGCCAGCTGGAAGCTCTTTCTCCAATGAGCAAAGTT
ACAGTGACCTGGCTGGAGTTACCTGGTGCACATAGGACCTTAGGGGAAAGTTCAGCGTGGACTACACTTG
CTCTGGGATCTGCTTTTCCACATGTGTGTATGGCACGCCTTTTTCTGCTGGATTGGGAAGGACAAGATTT
TGCTGTGCTAGGGAGAAATGAAAACGGGGTGAGCTGAGTAGCTGGGTTTCTGGAGGATAGAACATCAGAT
GGGGAGGCTTTCCGAGGTGAAGAATGAGAGGGAACCACTTACTAGAGAGAAAAGAGCTCCAGGCCTGGGG
AACAGCACGTGCGAAGGCCAGGAGAGAAGAACTGTTGAAACAACGAGAAGGGTGGCACGGCTGGAGCTGA
GCCAGCAAGGGGGATCGTGAGGAGCCTTGGGGTTGGGGAGATCTGCAGAAGCATCAGACCAGGCAGGGCC
TCGTACGCAGTCCTGAGGAGTTTTACTTTTATTCTAAGACAGTTGGGGAGCTCCAGGAGCTGTTTTAAGT
TGGGGAGAGACTGGATTCCAGCCTGCAAAAGCTGTTTTGTGAAGACTAAAACCAGTGAGGAGAGGTGGAG
GTGCTTTGGGGACACTGAAATGGATTCTTGGAAAGATTCTGAAGGCTGTGTTGAAAAGACACCTATAGCT
GTGGGGACATGACTATAATCCCAGCATTTGGGGAGACCGAGGCTGGCAGATCACTTAAGGTCAGGAGTTT
GAGACCAGCCTGGCCAACATGGCGAAACCCCATCTCTGCTAAAAATACAAAAATTAGCTGGGTGCAGTGG
TGCATGCCTGTAGTCCCAGCTACTCAGGAGACTGAGGCGGGAGAATTGTTTGAACCCTGGAGGCAGAGGT
TGTAGTGAGTCGTGATCACACAACTGCACTCCAGCCTGGGCAACAGAACAATACTCCATTCCCTCCCCTC
TACCCCACCAAAAAAAAAAAAAAATCCTGCCCTTAGATGAGCTCTAGGGCTGCTGAGTACAGTTGTCCCA
GTTGCACAGTGCCCAAGGGTTTGGCATTGCTAAGAAGGCCACGTGCAAATCCTAGATATTGAGTGTTGTA
TGTTTGTGACGTTGGTTTCCCGACATGTGAATGGCCCAAGTGTCTGGAAGAAGTGGCGCCACTTTCTAAT
TTGCTTGGAGATGTTGCATGTCCCTTAAATTCAGACAGGTGCAGGTAACTGGAGGTTCTGAACCAAAGGT
TAAAATGCAAATTCTCATACAGGGTTGGGAAGTTGTAGCCAGGGATAAGCTTATGTGACTGTTATATGGA
CTGAGGAGCAGATGTGAATTTCGAACCATGACATGGCTGAGGGTAGGGGTCGGGTGGATGGATGATTCAG
GGTTGTAACCCATAGAGCCCAAAGGGGAAGTGATCTGTGACCTGGGGTGAGGGTGATCTGGAAGATTTTT
GGATGGCTGGAAAGAAATGGGGAAGTCGAGCTGCCTGAGAGAGCCAAGTTATTTCCCAAAAGATTCCTTA
GGAGTCTTTCTGTTCAAGACCTCCGTGTGTGTGTGTGTGTGTTTAGGGTTCCCCAGCAATGGCCCAGGCA
TGTGAAGGAAACAAGCTTCTTCAGGGAATATTTGTTGAATGAGTTTTCCTGACTCCCAGGCTAGAACTGT
TTTTGCAATTTCCACCCTCTTTTCTTTCCCCCAGAGAACTCCTATTCGTCCTTCAAAACCCATCACGGAA
ACCCCTCTTGGAGAAAACCCTCCTTCCTTCCCCTCAGGACTTTCCCAGCCACCGTCTCTCCTCCAGTCCA
GCCTGATGCCATGGGACTGGGGGTTTCTCTGTCCAGCTCTGTTTCTCCCAGACTGGGGTCTGAGGACTCT
CAGGACCCCCAACTTTACCTAGCACAGGCTGGGCACAAGTGGGTGACAGGGAGTCTACGCCTAGTGGAAT
TATGTATTGGGGCAGGGTCAGTGTGAGAATACACATCCGCATGCATGTCTGTCCATGTCTGTCCGTACCA
ACCTTCCCCTTCCACACGGACCTGGGCACATAGGAGGTGTCTGAGCCTGACACATGGGACAGAGAGTGGA
CATGGCTGAGACACGGACAGAGAAAAGACAAGGAGTCCAGGGGGCTGAAAGCCTTTTGAAATCAGGAAGT
TCCTGTATTGGCAGAACAAAGCCCAGAGAGGAGCAGGGCTTTCCTCAACGCCACCCAGCAAGTGGACACA
GAGCCCGGCCTTGGATGACACCTCCAGGGTTCTGAACCCTGGACCTCGCTTTATGCAAGGAGCTGGCCCC
ACATTTCCATGAATCGGGGAAACAGCACAAGAAGGTTGGCCTGTGGCAGGGCAAGGGTTAAAGGGGTGAC
ATTGAGGGATGCCTCAGAGTCAAAGTCCCCTGACCAAGAGGAATAGAGTAGAAAACACAGAGACAGAGGG
TGAGATCACGCCCCGATGAGGACGGAGAGAGACAGAGATGGAGAGAGACATAGAGGTGGAAATATACAGA
GAAAGATAAATGCAGAGACCAAGGCAGGGAGTGTCGGGGGAAGTAAAGAGGGTGTCCTGAAGAAAGAAGG
ATCTGTTCACTCTTACCAGTCTGTCCTCGAATGATTTGCATAAAATGAGGAGGTGCCTGTCCACACCCCC
AATTCCTCTCTCAGGCCCCAGAGCCTGAGACCTCACCATGCCCCCATCAGAGATGCAAAAAACTAAACAC
CCAACTAGAAATCCTTGGGACCTCTCTCGGCTGGGATCTCAGAGCCTTTCTGTCCCCTACCCCTACCCCA
TGTGCTGTCGATTTTGCAGATGGGGACAACCTGGGGCCTCCCGGAACTCTGCCACCCTGGGGAAGTTGGG
GGAGGGCCTTAGTCCCGGATCACAACCCCGTCTGCTCCCCAGAATCCTTTCCTAAGAATCGTTGAGGACC
AAAGTTGTCTTTGCTGACACGTGTTGCTTTTCTCTTTGCCTTTTATTGTTTCAGAGAAAAATCAAGTTGA
CTGTGTCAAGTAACACCCCACCCCTTACCCCCGTCCAGCCATAGTGGCTCTCTGGAGACACAGGTCACAG
GCGGAGGGTCCCCTGATCATCCCCAACCACACAGCCAGGGGGACTTGACCCCTGTCCACCCCTGTCTCGT
GCTCCCTCAGACCCCCACAAACCGGCCAAGCAGTCCGGGGAGGCTTCCCCTCCACACAACTCTTAGCATG
TGATTGCAGATGTGAAATCAAAACGTTGTTTGTTTTTTGTTTTGTTTTGATTCTACCCCGTCGGTCCAGT
GTCTGCACAGACGCCTTCATTTCTCTGTAAATATGTGACTTGGAACAAATGTTTAACACAAACGAGAAGT
GGTCATGAATGCATGGTGTTGAGATGTTTTGCACTATTCTGACTTTTTGGTCTCTGTAAAAATATTTTAT
TAACAGCAGACATTAAAAAAAGAAAAACCACACACAGCCTTGGA.

In various embodiments, a UNC13A transcript is a pre-miRNA UNC13A transcript. In various embodiments, a UNC13A pre-miRNA transcript comprises a sequence provided as SEQ ID NO: 9594.

(SEQ ID NO: 9594 (SOURCE NCBI Reference Sequence NG_052872.1))
CAGTGGCTCACACCTGTAATCCCAGCACTTTGGGAGGCCGAGGTGGGTGGATCACTTGAGGTCAGGAGTT
GGAGACCAGCTTGGCCAACATGGTGAAACCCTGTCTCTACAAAAAATACAAAAATTAGCCGGGCATGATA
GAGAGTGTACCTGTAATCCCAGCTACTCGGGAGGCTGAGGCAGGACAATCCCTTGAATCTGCGAGACAGG
TTACAGTGAGCTGAGATCTCAACACTGCACTCCAGCCTGGGCGACAGAGCAAGACTCCATCTCAAAATGT
ACCCTGTAACAATATATAAGATGTCAACATTAGTAGAAGCACTCAAGCCTAGATGATAGAGAGAGATTCA
GTCTCAAAAAAAAAAAAAAAAAAAAAAAGAGAAAGAAATGGATAAAAGACTTGAACAGGCATTTCACCAT
AAAAAGAAACCAGTAATCACATAAAAAGTTCAACGTCAAGAGCAATCAGAAAGCAAACCAAAACCCAAAT
GAGGCACTCCTTCCCTCCCTCCACATAGGCCCAGTGAAAAAACTTGGACAATTCAAAGTGTTGGCAAGAA
CTTAGAGCCAGGCACGGTACCTCATGCCTGGAATTCCAGCACTTTGGCAGGCCGAGGCAGGAGGATCACT
GGAGCTCAGGAGTTTGAGAACAGCTTGGGCAACATAGGGAGACCCCATCTAAAAAAAATAAAATGAGCCA
GGCATGGTAGCACAGGCTTGTAGTCCCAGCTCCTTGGGAAGCTGAGACAGGAGGATCGCTTGAGCCCAGG
AGGTGGAGACTGCAGTGAGCTGTGATTGCACCATTGCACTCCAGCCTGGGCCTCAGAGTGAGACTATCTC
AAGAAAAAACAAAAACCAATAACAAAACAAAAACAAAACATAGAGGAACCGATGATTATACTGTGTTGGT
ATGATGAAAATCGCCAGACTCCCAATAAAAAACATGACATTATCAGGTGAAGCTGAGCATATATGTATTT
TCTTTTCTTTCTTTCCCTCCTTTTTTTTTTTTTTTTTTGAGACAGAGTCTTGCTCTGTTTCCAGGCTAGA
GTGCAGTGGCGTGATATCGGCTCACTGCAACTTCCGCCTCCTGGATTCAAGCAATTCTCCTGCCTCAGCC
TCCTGAGTAGCTGGGACTACAGGGGTGTGCCATAATGCCTGGCTATTTTTGTGTGTGTTTTTAGTAGAGA
CAGGGTTTCACCATGTTGGCCAGGATGGTCTCGATCTCTTGACCTCATGATCTGCCCATCTCAGCCTCCC
AAAATGCTGGGATTACAGGCATGAACCACCATGTCTGGCCACATATATGTATTTTCTGACCCAGCCATCC
TTACTCATGTGTGCCAGCACACAAGTCTGAAGTCTGAGGATGGTCATAAAAACTTTTTTTTGTAATAAAC
CTGAGCTTGAAACAACCCAAAAGTCCATGACCTGGAGAACAGATGAATAGCCAATAGAAGATTCATACAA
AGGAATACTATGCAGCAGTAAAAAATGCAAGCCTTACAGCCACATGCAGCGATATGGATGAATATCAGCA
GAATCACATTGAGCAAACAAAACAGATTAGCAGTCCAGGTGCTGTGGCTCACACCTGTAATCCCAGCACT
TTGGGAGGCTGAGGTGGGTGGATCACCTGAGGTCAGGAGTTTGAGACCAGCCTGGCCAACATGGTGAAAC
CCTGTCTCTACTAAAAATACAAAAATTATCACTGCAACCTCCATGGCCAGGCATAGTGGCTCATGCCTGT
AATCCCAGCATTTTGGGAGGCCGAGATGGGCAGATCACCTGAGGTCAGGAGTTCGAGACCAGCCTGACCA
ACATGGAGAAACCCCGTCTCTACTAAAAATTACAAAAAAATTAGCTGGGCATGGTGGCACATGCCTGTAA
TCCCAGCTACTCAGGAGGCTGAGGCAGGAGAATTGCTTGAACCTGGGAGGCGGAGGTTGCGGTGAGCTGA
GATCGTGCCATTGCACTCCAGCTTGGGCAACAAGAGCAAAACTCTGTCTCAAAAAAAAAAAAAAAAAAAA
AAAAAAAAAAAAAGCCAGGCAAGGTGATGGGCGCCTGTAATCCCAGCTACTCGGGAGGCTGAGGTAGGAG
AATCGCTTGAACCTGGGAGGCAGAGATTGCAGTGAGCGGAGATTGTGCCACTGCACTCCAGCCTGGGTGA
CAGAGTGCAGCTCTGTTTCAAAAAACAAACAAACAAACAGATTAGTAAAGAATGTGAACAAAGTCAAATG
AGGTGGCTCATGCCTGTAATTTCAGCACTTTGGGAGTCCAAGGCAGGCAGATTGTTTGAACCCCAGAGTT
CAAGACAAGCCTGGGCAACATAGTGAGACCCTCATCTCTAATTAGCCAGGCATGGAGGTGCATGCCTGTT
GTTCCAGGTACTTGGGAGGCTGAGACAGGAGGATTGTCTGAGCTCAGGAGCTCACACCACTGTACTACAG
CCTGGGTGACACAATGAGACCTTGTCTCAAAAAAATAGAAACATGCAAGAGGTAACCATGTATTGTTTGC
TAAGGAGCATGTGGTAAACTATAAGGAAAAGGAAGAAAATGAAAAACAAAATTTGTGATAGTTCTTCCAT
CTGCAGGGAAGGATTATGAGATTGGGGAGAGGCACATGGGGGTCTTTTATTGTGATGGTATTTTTTTTTT
TTTTTGAGACGGAGTCTCTCTCTGTCACCCAGGCTGGAGTACAGTGGCCCGATCTCGGCTCACTGCAAGC
TCCGCCTCCCGGATTCACGCCTTTCTCCTGCCTCAGCCTCCTGAGTAGCTGGGAATACAGGCGCCCGCCA
CCACGCCCGGCTAATTTTTTGTATTTTTAGTAGAGACGGGTTTTACCGTGTTAGCCAGGATGGTCTCGAT
CTCCTGACCTTGTGATCACCCCGCCTCAGCCTCCCAAAGTGCTGGTATTATAGGCGTGAGCCACCGAGCC
CGGTCGATGGTAATTTTTTTTATCCAAGCCTGCTCCTCATAGTTTATCTACTCATTGATGTTTAATAAAA
CATCAATGTTTTATTGATTATCAATGTTTAATAAAACATCTACTCATTGATGTTTAATAAAAATTTAAGA
CCAGGCATGGTGGCTCATGAGCCTGTAATCCCAACATTTTGGGAGGCTGAGACGGGAGGATCACTTGAGC
CCAGGAGTTTGAGACAGCCTGTGCGACATGGTGAAACTCTGTCTCTACAAAAAATACAAAAATTAGCTGT
GCTGATTTTTTTAAAAATTTAAAGCAGTGACAGATACAAACATGGTTACTATTAGGCATTAAGATTACAA
CTGGGGCCAGGCGCGGTGGCTCATTCGTGTAATCCCAGCATTTTGGGAGGCCACGGTGGGTGGATCACTT
GAGGTCAGGAGTTTGAGACCAGCCTGGCCAACATGGTGAAACCCCATCTCTACAAAAAATACAAAAAAAT
TAGCTGGATGTGGTGGCGAGCGCCTGTAATCCCAGCTACTCAGGAGGCTGAGGCAGGAAGATCACTTGGA
GGTTGAGGTTGCAGTGAACTGAGATGGTGCCACTGCACTCCAGCCTGGGCCACAGATTGAGACTACACCT
CCAAAAAAAAAAAAATTACAACTGGTTTCACATGTTAACTCATGTAATACCTCCAGCAACCTTGTGAGGC
AGGACTTGTTATCATATTCATTCTACAGGTGCGGAAACTGAGGCACGAGGAGGTCACAAGACCTGCCCAA
GGCCATGCTTGTGATGAGAGACAGAGTTTGGTTTTGAACAATGGCCTCACTCAACTCCATGCACATTCTG
CATTTATGAAGCTCTTTCATGTGGGCTGGGGGCAGGAGACCCTGTGGGGCTCAGCCTTTCATGCTAGTTT
AGGTGCCAGTGATACAGCGAGCAGGTTGCAGGGCAAGGGGCCCACATGGAGCTGCCCAGTTTGATATGTG
GGGAGGGGGCCAGGCGCAGTGGCTCAGGTCTGAATCCCAGCACTTTGGGAGGCCGAGATGGGCGGATCCC
CTGAGGTCAAGTGTTCAAGACCAGGCTGGCCAACATGGTGAAACCTCGTCTCTACTAAAAATAAAAAAAA
TAAAATAAAAAAAAAAAGCTGGGCATGGTGGCATGCGCCTGTAGTCCCAGCTACTCGGGGAGGCTGAGGC
AGGAGAATCTCTTGATCCCAGGAGGCAGAGGTTGCAGTGAGCCAAGATTGCGTCACTGCATTCCAGCCTG
GGCTGCAGAGGGAGACTCCATCTAAAAAAAAGAAAAGTGGGGAGGAGTGGCAGAGTTTCGTTCGGGTCAG
GGCCCAATCAGAGGAAGGGATAGGGGCTGGGGGAGTCCAGAGAGGAGAAACAAAGGCTCAGCTTGGGGCA
GTCAGGGAAGCCTTCCTGGAGGAGAGGATATTCAGACAAGACATTGATGGATGAAAAGGAGTTCAGAGTG
TGGGCAAGGACCCATGGGCTGGATGGCTTAGAGACATAGAGAGAGACCTAGACAGGATGCAGTAGAGCTG
CAAAAAGACTTTACAGAGAGAGGCTAAGAGACACATGACTGAGAGAGCAGTGGACAGAGTCAGAGGAACA
GAGACCCAGGGAGAGAAAGAGACCAGGCAGTAGAGTTAGAGAGACAGAGACGTAGAGACAGAGGAAGAGA
GATACTCAGAGAACAGGACCATCTGCCCGGTGCTGGTGCCGGGCGTACACACAAGAGCTTGCATTTGCCT
GTACTTGCTTGAGTATTGCAGGGAGGCGTTTGCCCAGGTTACCAGGGCAGGAATGTCCATGCATGGGTGG
CGGGGGCACGTGCACCATTTCAGTGCAGGTGAGGGTCTGCACAGGAGGAAACCTGCTTGGGGGCCGCCGT
CGCCCCTTGTCCCCCTGCAACGAGGAGGTGTGGGGCGAGGCGCCCCTTCCCGGAGCCAGGCCCTTTAACG
CCCCCCCAGCCCGCAGCGCCCCCGCCCCATCAGGCGTCCCTGGGCTCCGCCTGCCCGCCCCGCATCAGCG
CCACGTCAGGCGCTGTCCGCGCCCCCGGTGCTGAACCAAGATGGCCGGTGGCGGCCGGGCCCCGGCGTGA
GCCAAGCGCGGGCTGCAGCCGGGAGATGCCCCAGCCCAGCGGCCGCTGAGCCCGACCCGACAGAGCCGGC
CCGGCCGCCTCCGGCCCACCTGCGAGCTCGGAGACATGTCTCTGCTTTGCGTTGGAGGTAGGAGGCGAGG
CTGGGGGTCGGGGGACCCGTGTGGACGCGGGCTGGGGTCGGGGTCCGCGTGGATGCGGCTCCCAGGGGGT
TCCTGGGGGGTGACCCGAGTCCAGGTGGCTGGACTTTTGAGAGGGGTAGCCCTTGTCGACGCAGCTGGGC
CCCGGAGGGGGTGATCCGTGTAGACGCGGCAGGGCTCTCGGGGGTCCTGGTGGCCGCTGCTGGAGGGGTC
GTGTGGACGCGGCTGAACGCGGAGGCCCGTGTGGACGTGGACGGGTATCCGGGGTTCTACCCGGAAACGA
TCTAGACTGGGGCGTCTGTGTAGACCAGGTTTTTAGAGGGTCAGTGAGGAAGAGTCTGGGCTCGGTAGTT
TGATTGAAGCCTGCAGGGGTGCTTGGAAGGGGTCTGGTGTTGTGGGCTGGGTTACGGGTCCTCATGGACG
CGGACCCGGCTGGAAATTCGAAGGAGAGTGCGGTGGGAGGGTTCTCCAAGCCGTGGGGATGTAAGATATA
GGTAGAGGGGCTTGGCCATCAGGGCATCCACGAGTTCTAGGACTCTCCCAGCCCTAGGTGTAAAGAGGAC
TGGAGTGGGGGGAGTGTGGAGACAGGAGACCCCTCCCAGCGTCCAGAGAGGGCTGTGACTGGAGGTCTGG
GGGCGCTTTTCCAGTTAACTCTTTGGCCTCCCGAGTGAGGGGGTTGGAGGAATGTCCAGAAGGTCTTCCC
GCTCCCTGCCCCCTCCGGTTCTCCCAAACACGTTTCAGGGCCTTCTCGGCAGGGAGCCTACTTCTTGGAA
GAGCAATGTCTCCTGGTCCCCCTCCTGGCCCATCCTCACCTCACCCACTTTGCACTGGAGAGAGGGAGGG
AGGGAGACGTGAGGTGGGGGGGGAGCTATTTTGGGGCCAGAACGCCATCTGCACAGAGGCCCACGCCCAG
GCCCACGCTCTGCGTTGGGGTTGGAGGAGGGAGGGAGACGTGGGAGGGGCATGACCCCCCACCCCACACC
TTTATGGCTCTGGGGTCGTGGGGAAGCCAGCCCAACCGAACCCAGGCGTTCTGGGCACTCCCGTTTGAGA
AATCAAACTCTGGGAGACCTTGGGCGGCAGGGGTGGATGTGGACCGCCGTTCCACGCCTACAGAGCTCTG
AACCCCTGGTTCTTAAGGACTGGGGTCCCCCCGTCACTCCCACCCATTTCAAGAAGGCTGGGGTGGGGGA
AGGGGTGTGTCTGCCAAGCCTGAGACTGCAGGCTTCGGGAGACCTCGGGCGACAGGAAGCGTGCGTGTGC
TGAACTCCTGCTGCCTGGACACAGGGTGGGCAGGCCGGGGAGCCGGGAACCGCCCCCGCCGCCCCTCCTC
CTCCCTCCCCTCCCCCTCGGCCGATTCATTCACAAACCCTGCCCCGCCCGCCCGCTCCCCGCCGCGGCAG
CTTCCGAGTGCAAATCCGGCCGGGCTGGGCCGGCGTGGACGCGGGTGTTCTTAAAGGGACAGGGGCCTAC
TTTCTCTAAGCTCGTTTCTGCTGGACGGGGCTGGGGGTGGTGGAAGAAGGGTCCCTAGAGTCAAAGGTCA
ACTCCTCCATCCCCTAGCCTCCAGGCCTATGGAGTTAGGGGAGTAGGGAAGTGAAGGAGACCCCTAGGCG
ACCCCCACCGCCTAGGCCTGAGGACGGAATGGGAGGAGAACTTGAATGTCCAGACCCCCTACTGACCTCT
CCCCCTGCTCTAAGTAGCCGGGCTCTTAAAGGTGGCACTGGAGGAGGTTCCGACGTGGGTGTTCTTGGCT
GACTCCTCTATCCTCTCTCTTCTCCCCCCAGCATCCCGCCCCACCCCGCCTCACATCCTCCAAGTTGGGC
TTCTCTGGAAGGTTTGCCTAGTGGAGTGGGGGGGGGGGGGGGGGGGGGGGGATCTCACCCTTAACTCTGA
CGCAAACAGGAGGCTCGGCTGCGGGGGTGGCATTAAGGGGGGGCAGCAGGAGGCAAGAGGGATGAGGCTT
CCAGAAACAGAGACGTTGGGGGAGGACTGTGCTGGATTCTGTGGACTTTACAGGGAGGGGCCCGGCTGAG
TGGAGAGGAATCGCTGGGAGGGGCAAGGCTGGGGAAGAGGTTGACAGGGGTGGGAGGTAAGGCCCTCAGG
TGTAAAGGGAGGTGTGAGGTTGGAGTGAGGAGGAGTAGGAGGCAGGGAGGGGTGGGGAGAGGGAGAAGTG
CTGGGTGAGGCCAGTCTCCCAGGGAGAGGTGGGATGGTGCCCAGAGAGGGGAGGGGCTGGGATGGCTGAG
CACGAGAGATGTTGGGGTGGGCTTGGCAGGAGTGAAGCTAGGCTGAGATGGGTTGCCGTGTTGCCTGAGC
TCAGCTGAGGAAATACCTGTCCCCCCTCCCCTCCACCGGGGACCCACAGGCGTTTGCCAATCCCTACACA
CATTCATACCCAGACTTGTTCCTGTGTGGACACAAATGCTTAGGATGTATGAAGGCTCTACGCAGACCAT
GTGCCTGGGAAGGCACATTCCGTAGGCACACGTGTGTACTCAAATGCCTGTGTGAACACAAGCATACACG
TGTATATAGGACACAGTCTGCCGCTACAGATATAGTCCCAGCAACACACGTGTGAGTGTGTGTGTCTGCC
AGCACGCATATAGATGTATCCACGTGTCTGATGAATGCTCACAATATACATGTCACATAAAGATCCACCC
ATGTCCAGCCGAGCGCGGTGGCTCAGGCCTGTAATCTCATCACTTTGACTTTGGGAGGCCAAGGTGGGCA
GATCACTTGAGGTCAGGAGTTCAAGACCAGCCTGGCCAACATGGTGAAACTTTGTCACTGCTAAAAAATA
AAAAAAATTAGCCAGGTGTGGTGGCGCGCACCTGTAGTCTCAGCTACTCGGGAGGCTGAGGAAGGAGAAT
CACTTGAATAGGGGAGGTGGAGGTTGCAGTGAGCTGAGATCCTGCCACTGTACTCCAGCCTGGGCAACAC
AGTGAGACTCTGTCTCAAAAAAAAAGATTCACTCATGTCCTAACACTCAGACATGTACCCATACATATCT
GTTAAGGTCCCCAACCCCATGAAGCACATATATATTCATATAAGCACATGTCAAAGCCATGTGTGTATGC
ACATGACAACACTTGCCTGGACATTCAAAGCAGCCCCTTTCAACAAGTGGATGATGATGCATACAAGTAA
ATACACATTTGAGTATTTCATTGCACACAAGTGCATACATGTATGCAGATAAACAAATACAGCTGCCACA
GAACTGCATCCATGTACACATGCAAACATCAATATGTGTGTTTTCCACCTAACATATCACCCACAAGCTC
ACACACACCAGTGTGTCTCTGATGTAGTGACACCCCACTCCTAGACACACATGTGCAGACTCCCACCTGC
ACTTAAATGGTGCACATACTCTGCACACATGCACACTCATATCCATGTAGAGGCAGGCACCTGCCTGCCC
TGGTAAGATGAGCCAACTACTGCTGCAAATACCCAAGCAAGTACAGGCAAATGCAAGCTCTTGTGTATAC
ACCCAGCACCAACACATAGCAGACCACCCTGTATGGTGTATGGGCCTTATCTCTGCCCGCTTCATTTATT
TATTCATGTGGTGCCCCGCTGCTTCCCAACATCGTAATCCACTGACTAGGTCTTGGCAGGATCCTGAGGC
TGTGAATGGTAAATGCCACCTGGTTCCTTCATGAAGTCACTTATTTTATTTATGCAACAAGCATTAATTG
AGCACCTACTGTATACCAGGCACTGTTCCTGGCTCTGGGACCCAGCCCTATTCCTAGCCATCACTCCTGC
ACGTCCCATGGGGTTAGAATGAAAGAGATGAGGGAGGGCTAAGGGAGCTGAGGAGTGGGGTATCCAGGGC
TCTCTTCCTCTGGGAAGCCTACCCTGATTCCACCACCACCATTTACTTGAGTCTCAGAAGATGTGATGAG
GGGGAGATGAGCAGGAGGGAGATGGTGCTCTGGGTGGAGGGAACAGCATGAGCAAAAGCATGGTGGTGTG
AAGGGTTACTTCCAGATCTTCCTATGTGCCTCACAGCATTTTGCACAGGCCACTTGATGTTTTGTTTTGT
TTTGAGACAGAGTCTCACTCAAAGCCTGTTACTCAGGCTGCAGTGCAGTGGCATGATCTCAGCTTGCTGC
AGCCTTGACCTCCAGGGCTCAAGTGATCCTCCTGCCTCAGCCTCCCAAGTAGCTGGGAGTATAGGCATGC
ACCACCACACCCGGCTGACTTTTTTCTTTTTAATTTTTTGCAGAAGCAGGGTTTTGCCCTGCTGCCCAGG
CTGGTCTTGAACTCCTGGGCTCAAGTGATTCACCCTCCTCAGCCTCCCAAAGTGCTAGGATTACAGGTGT
GAGCCACCACGCCCAACTGATGTTTTAAAGGCTCTATAAACATGTTATGAATAAATGACATGAACAAATC
AGTGAATTTAGGTGAGGGTGGGTTTGCTTCACAACTTACAATAATATCAACAACAAACCCCATTTATCCA
GCTTTTTTGTTTGTTTGTTTGTTTTTTGAGACACGGTCTCACTCTGTCACCCGGACTGCAGTGTAGTGGT
GTGATCTCGGTTCACTGAAACCTCCACCTTCCAAGTTCAAGCGATTTTCCTGCCTCAGCCTCCCGAGTAG
CTGGGATTACAGGCACGTGCTACCACACCTAGCTAGTTTTTGTATTTTTAGTAGAGATGGGGTTTCTCAT
CTCTACTAAAAATACAAATACATCTCTAGTGGCCAGGCTGGTCTTGAACTCCTGACTTCAAGTGATCCTC
TTGCCTCGGCCTCCCAATGTACTGAGATTACAGGCTCGAGCCACCGTGCCCAGCCCAGCATTTCATAGGC
ATGATGTTAAGGATTTTTATGCCCATGCCCTCTTTAACTCTTACAGTAGCCCCATTGTACAGATGAGGAA
AACTGAGGCTCAGGGAGCTTTAACAACTTGCCTGGTGGAACAGAGCCAGAGGTGGAGCTGACTGACTCTA
GAGCCTACTGGGGTTGGCAGAAGTTTGAGCCTTCTCTCAGACCCCAGGACCTCGGAGACAGCCAAGTCCC
TTCTGTGGAGTGATGTTCTGCCCTGGGATTGCCTTCTTCTTTCTATTTATTTATTTATTGGAGACAGAGT
CTGGCTCTGTTGCCCAAGCTGGAGTGCAGTGGCACAATTTCAGTTCACTGTAACCTCCTCCTCCCGGGTT
CAAGCAATTCTCCTGCCTCAGCCTCCTGAGTAGCTGAGATTACAGATGCACACCATCATGCCCAGCTAAT
TTTTTGTATTTTTAGTGGAGATGGGGTTTCACCATGCTAGCCAGGCTGGCCTCGAACTCCTGACCACCAA
GTGATCTACCTGCTTTGGCCTCCCAAAGTGCTGGGATTACAGGCATGAGCCACTGTGCCCAGCCAGGATT
GCTTTCTTCTCTCATGCATCTAGTAAACAGACATGTCACAATCTCTCCATTCATCGGGGCTATTCCCCTG
CTCCTTGCCTAAGAAGCCTTCACACATCCCTCCGTTCCTGGCTACTCAGTCCCTGATACCCTCCAGCTAG
GAGGAGCTGCATCTAGGTCTTAGCTCCTACCGTTCTCATGGGATGCCCTCCAGAGTTGCATCTTCAGGGC
TGCAGCCTAACCCTTTCCCTAACCTGGCCTCACTTTTCTTCCACATACAATGGACCTCTTCTGCAAGGTG
CCAATAAGGGCATCCTGGGTCCAGCAGGCCTGAGTGTGAATTGCTAAGTCCACCACTTCCTGGCAAGTCT
CACATCCTCTGCATTCCTTGGTGTGCTCATCTGTGGAATGGGCACAGGAGTAGCATCCACCATCTACGTT
TGCCAGAACTCTCTAATGAGGCTTGCAGAGCCCCTGGCATGACTCCTGGCCTACGGGAAGGCCCAATAAT
CGTGAGCAAAAGACATGAAAAATCAATGATGGGCCGGCCCCGGTGGCTCACGCCTGTAATCCCAGCACTT
TGGGAGGCCTAGGCGGGCAGATCTCCTGAGCTCAGGAGTTCAAGACCACCCTGGGCAACATGGTGAAACC
CTGTCTTTACTAAAATACAAAAAAATTAGCTGGGTATGGTGGCGCGCGCCTGTAGTCCCAGCTACTCAGG
GGGCTGAGGCACGAGAATCGCTTAAGCCCCGAAGGCGGAGGTTGCAGTGAGCCGAGGTCGCGCCACTGCA
CTCCAGCTTGGGCTACAGAGTGAGACTCCGTCAAAAAAAAAAAAAAAAAAAAATCAATGATGATGTTTTT
ATTAATGGAGACTAAAAGCTTTCTGGAAGGCGGTTCTGAGTGCAGGACATTTCTGAGAGTCCACTGCATT
CTGGGGCCCGAGGAGCGCTCCAAGAGGGGTCCCCCAGGGCCACTCCAGGAGGGAGCCTAGTTCGTTGAGG
GTACACGGGTGGGACCGAGCATTCAGCACCGTGGACAGCGCCTGCCCGCGCCTCTGGGGGACAGCTCTTA
GCTCAGCCTCCCAGGATTAATGGAGGAGGTGACGTCATTCCTTCCGTGAATTACTATTTAATGAGCACCT
ACTATAGGCTAGGCCTACTTCTAAGTGGTTCATGGTAAACAACACACACACAAATCCCTTATTCCCGAAG
CTCTCTGGAACTCCAGGTTCTTGGTGTTTTTTACACTGAAGGCTCAGTCTGGCTCTGGTCTATTTGTGTC
AACGGAGGATAAGGGTCCTCATTTAGTGTCTCCCAGATGCGAAACCAGCACAGCTGAGGTCTTTTATCCT
AAAGCCCATCCATCTCCCGCTGAAATGCCCTCCACCAACATCACACACAAAACGATCAAATCTCCTATAA
TCTATTTCTGGATTCTCTATGCTTCCTCCCTTCCCCCAAGCTAATCCTGGGGCGGGTGGGCTTTCAGAAG
CCCTAGACGGCTGGATCTCCACCTCCTGTCTCCCCACCCCATCCCACGTGGCAGGTCAGGGAGCCAGTGC
CCCAAGACTGCAGTCCCCCCTAATTAGAATCCAGGGTCAGTCAGCCATGTGGTTCACGCCTGTAATTCCA
ACACTTTGGGAGGCCGAGGGGATGGCTTGAGCCTTGAAGCCAGCCATAGCAACATAGCAAGACCCCATCT
CTACAAAAAAAAAAAAAAAAAAAAAGGAATAGCCAAGCATGGTGATGCGCACCTGCAGTCTCAGCTACTC
GAGAGGCTGAGTGGGGAGGATCGCTTGAACCTGGGAGATCGAGGCTGCAGTGAGCCATGATCACACCACT
GCCCTCCAGCCTGGAGCCTGGGCAACAGAGAGAGACCCTGTCAAAAAAAAAAAAAAAAAAAAAAAGAAAA
GAAAAGAAAAAAAAAGAAGAAGAAGAAGAGAAAAGAAAGAAAAAGAAAAGAATCCAGGGTATTCAAATCC
TAGAATCCAGGCCAGGGACACCTTTAGAACACAACCAAATAATAAAAACAGTAAAGATATCAGTAATAAT
AATAATAGTATAAATGACACTATAATCATTCTGCAGAAGGGGTGAGGGAGGAAGAGGAGGGAAGAAGAGG
AGGAGAAAGGAGAGGGAAGGGAGATCAGCCACTCCGTCTCCCCATTTTCCAGATGCAGAAACGGAGGCGC
GAAGCCAGGCTGCAGGGGTGTCCCGCAGCCTGAACTCCTGCCTCAGCCTCCCCGGGTACGGCCACTACAT
TCATCAGTCAGCGCTTCTGGGGGCCCCAAAGACCTTTGAGGCCGCATGACGCGGTGTCTTCACCCGCTCG
TCGCTGGGGACAGCAGTGGGCACCCCCCTCATCCCCGCCCCCCTGTCCGCCGGCGCGCCTCCCACGCCCT
CCGCGCGCCCACGCTATAAATAGCCTCATGCGTGTGACCCGAAGCCCGCTGGGGAAGGGTGGCGCTTCCG
CCCCGCTGACCCTGATACAGCAACCCGGCGCAGGCGGGGAGGTGGCGGGATGTGACGACGAAGACGGTGA
CTCAGGGACCATTTACTGACCGGGGAAGACAGGGAGAGAGACTGGGTTCCTTCTCTGAGCCCCCTTCCTC
CTCCAAGCCTCTCCCCCGATACCACCCTCCCCTCTGCGCCCATCCCCCTTCCCGAATCCCCTCCCTCACC
GCGAACACCCTTCCCTCCTGAGAACCCCCTCGCCACTCTCGGCCCCCTTTCCTGTCCCCTTTTCCCTCTG
AGACCCCTCTCCCACTGGGAGCCCCAGTGATGAAAAATCATGGATGTCCTCAACCTGGACCTCGGCAGGG
AGCTAAGTGCAGGAGCCTGGCTTCAGCTATCAGAGGGTACAGCCCTCCAGCCGGTCCCCCACTGCCTGAG
GACCCCAACGGAGACCATTCCCCTCTAGCCAAGAGAGGCTGCAGGAAGGAGCTGAGGCTTAGGGAAGAGG
AGCCGGACTGACCAAGGGAGGGGCACAAGAAGGGCAGAGACAGTGAGAGAGAGAAGGAGAGAACAGAGAC
CAAAAGAAGACGACCAGAGTGGAGAGAAATAGAACGGGGAGGAGGGAGATGAGAGACAGGGATTGGGAAA
GAGAGAGAGAGAACAGATCAAGGAGCTACTGGGCTCCCCGCTGAGCCTAAATCCATGGTTTATAAAACAG
GGCACAGTTGGAGGAGCTTCAAGTGAGCCCAGCCCAGTCTAGACCCCAGTGAGCCCCTTGTATCACCCCC
CAGAGCCTGTCACTTTCCCACCCCCATATGCATCCCTACTTTTCTCCCCCAACTGCCTCTCCTCTCCCCA
GTGTCTCCTCCACCTGCCTGCCCTCCCTAGTGGAAATCTTTCCCCCTCTCTCTTTTATTATTATTATTAT
TATTATATATTTTTTGAGACAGAGTCTTGCTCTGTCGCCCAGGCTGGAGTGCAATGGCGTGATCTTGGCT
CACTGCAACCTCCGCCTCCTGGGTTCAAGCGATTCTCCTGCCTCAGCCTCCCGAGTAGCTGGGACTACAG
GCGTGCACCATCATGCCCGGCTAATTTTTGTATTTTTAATAGAGATGGGGTTTTACCATGTTGCCCAGGC
TGGTCTCGAACTCCTGACCTCATGTGATCCACCTGCCTCGGCCTCAAAGTGCTGGGATTACAGATGTGGG
CTACCACGCCCAGCCTCTCTCCCTTTTAAATTATTATTTTTAGACATGGGATCTTACTACATTGCCCAGG
CTGGTCTTGAACTCCTGGGCTCAAGGGATCCTCCTGCCTTAGCCTTCCTAGTAGCTGGGATTACAGGCAT
GTGCCTCTATGCCTGGTACTCTCGCTGTCTCCTTGTGTCTCTCTATTTCTCTCCATCTGTCCAGACTCTC
CTCTCTTGCTTTCATGCCCATCATTTCTTCCTCTCTCCTCTCCCTTCCTCTCCCTGTATGAGTCCCTCCA
GCTGTGGGTGCCTTTCTCAGCCTCTGTCAGCATCCCCTCCACCCCCGATCCCGTCACATTCTACCTCTTG
CCCCTTCCCCTGCCCACCCCCTTCACAGAGAGTCCCCACGCCTGCAGGCTGGACCTCCTCCAGGAGCCAG
GCCCAAAGCTCCTGCTGGGCACATTCCTGAGTGTTCCTGAGCCTCTTTCAGTCCCAGGGGTGAGGTGCTG
GGTGGGTGCGCTCCCTGGGGGCTGGGTGGGGCTGAGGATGTGAGCGAACAAACCAGACCTAGAAAAAAAA
AAGACAGAGATGGAAGCAGAGATGAAGGCCCAGAGAGAGGGGTTCACTTGCCCAGGGTCACACAGCTCAG
GAATCTGAACCTGGCTCAAACCAGCATTCACTTCTCTCAAGGCCCACCCAAGGGCTGCAGTGCAGTGGCA
CAATCTCAGCTCACTGCAACCTCTGCCTTCCAGGTTAAAGGGATTCTCCTGCCTCAGCCTCCCGAGTAGC
TGGGATTAGAGGTGCGTGCCACCACACCCGGCTAATTTTTGTTATTTTAGGAGAGACGAGGTTTCCCCAT
GTTGTTCAGGCTGGTCTCGAACTCCTGACGTCAAGTGATCCACCTGCCTCGGCCTCCCAAAGTGCTGGGA
TTACAGGCATGAGCCACCGCGCCTGGCCAGGAACAGTCTTTACTGACATGTGTTTAGGGCCATCGCACTT
CCCCCGCTCTTCACGGGAATTCTCTCTTCACTGGCTCCACTCAGGTGCCTTTTGTGAGACAGAAATTCAA
GCGATGAGGACAGAGATACAAACCCGGGATGGGAGGCTGTGACATCTGCCCATAGAAACACAATTCTGTT
TCTAGGAATTTATCCTTCAACTCGCTAGTACACGCTCTTCAAGACATACTGAAAAAACATGAAGGATTAG
AACAGTGGGTGTCGAATGCTACCATTTGTCTTGGAAATCAAGCAAAACGAACGTGGGTATGGTTGCCGAC
GCATGGACCATCTCCAGTGGAACAAGGAACTGCCAACGTTGATTGCATTTGGGAGGGAGATGAAGGGCTG
GGATGGAAAGAGCTTTATTCTCAGTGCCTCATCTTTTGGTTGTTTTGGATTTTACAACATGTGCATGCAT
TGCCTACACAAACAAAGACACTAATTTGAAAAGGAAAAGGGGAGAAGAAAAGAGGGAGGAAAGAAAAGGG
GCCGGTGTGGCAGCTCACACCTGTCATCCCAGCACCTTGGGAGGCCAAGGCGGATGGATCACCTGAGATC
AGGAGTTTGAGACCAGCCTGGCCGACATATACTGAAACCCTGTCTCTACTAAAAAATACAAAAATTAGCT
GGGCATGGTGGCGCACGCCTGTAGTCCCAGCTACTTGGGAAGCTGAGGCAGGAGAATTGCTTGAATCTGG
GAGGTAGAAGTTGCGGTGAGCTGAGATGGCACCACTGCACTCCAGCCTGGGCAATAGAGCAAGACTCCGT
CCCTAAAAAAAAAAAGAAAAAAAAAAAGAAAAGAAAGAAAGGAAAGGGAGGGAAGAGCTCAGTGTGGGAG
AAGATTGGGGGTGGGAGTAGAGAATTAATGGAGGAAAAAGATGTGGGGGACAAAAGCAAGGTGTGAGAGG
TGTTGGCAATGGAAACTGAATACCTGTTGAGAAGAGCCAGGAGGGGGCGACATGGCTTTGAATCTCTTGG
GGCTGTGAGATTATGTGAAATGTGAGAAGACTGGACCTCAGTGGGGTGCCTGACATATATTCAGGTTACA
CCTCCCACAAGCAGGTGGGGCTGGACAATCTCTGTCAACCCTCCAGCCAGGACCCAAGTGTCTCCCTGTA
CATTACTTTTGGTTGTTGTTGAGACAAGAATCTCACTCTGCTGCCCAGGCTGGAGTGCAGTGGCATGATC
TTGGCTCACTGCAATCTCTGCCTCCTGGGTTCAAGTGATTCTCCTACCTCAGCCTCCCAAGTAGCTGAGA
CTATAGGCGCCTGCCACCACACCTGGCAAATTTTTGTATTTTTAGTAGAGACAAGGATTCACCATGTTGG
CCAGGCTGGTCTTGAACTCCTGACCTCAGGTGATCCACCTGCTTCAGCCACCTAAAGTGCTGGGGTTATA
GGTGTGAGCCACCGTGCCTCGCCCTCCACATGCATTTTGAGAGCCAGCTCTCTGGGTTGCTTTGTCCCAA
CTCACCCAGCCTTCTTTGAGGCTTCCCCAAACCCTGGGAGCATTTGACATTGCAGACTGAGCTGGACAAT
CAAGGGAGAGGTCTTGCTGACCTGTCGGGGCTGGTGGAGTCATCAGAGATTGTCTTTATCCTCGCCTTCT
TCTTCTCCACCTCCTCAACTTCCCCTCTTCTTCTGGGGCAATCCTGGAGGGCTTCTTGTAGGAGGCCTCT
CCCACCATCTGCCCAGCTCTTTCTTTCTAATCTTGTTCAATTGTGGGTAAAGGCACAGCTAGTTGGGAGC
TTGGGGAGTTTGATGGGGACAGGGGATGCAGGGAGAGGGAGGGGCAGGGAATGCTCTCAGGGTCCAAATT
TCCTTCCATTCTAGGGTTGCCTGTGCTCATCTGTCTGATGGCTTCTCTCTCTCCCTCTCATCCCTGCCTC
CTCTTTCCTACCTCTCCTCCTCCCCTTGTCTCTGTCTCTGACATTGCTTCTATCTTTCTTTTTTAACATC
TGTCTCTTTTGCTTTTTATCTTTGCCTGTCTCTCCTCTGTGTCTCTCTCCTTATGTCTCCATCTTGGTGT
GTCTCTGAATCTCTCCGTATCTCTGCCTCCTCCTCCCCACATCTCTCCCCACCTCCCTTCCCTATCTCTG
TCTCTCTCCACAGTCAAAAAAGCCAAGTTTGATGGTGCCCAAGGTAAGTGGCTTCTCTTGCTGTCCTGTC
CCGTGTTGTCTGTCTGTCTGTCTGTCTCTTGGAGAGACTGGGAGGGGGTCCCAGCCCAGACTTAGAGTTG
GGCTTTATGTCTTCTGAGCATCAGGAGAGGGGTCAGAAGATTAAATAGGGGGAGGGGCAGCCATTGGCCT
GGGGTCCCTGCAGTTTGTATGGGGGTCCCCGGGTCTCCCCAGCCATCCTTGGAGAGTCTGTAAGGGGTGG
GGCTTGCCCTGAGCCAGTGGCTCTTCCCAGGGTCCTAAGAGGTAGAATGGGGACTCTTAGATGGGGCTAC
AGGTGGGAAAAATGAGGCTCAGAGAGGCGAAAGGAGTTTCTTTCTTTTCTTCTTCTTCTTTTTTTTTTTT
TTTTGAGACAGAGTCTCCTTCTGTTGCCCAGGCTGGAGTGCAGTGGCGTGATCTCGGCTCACTGCAACCT
CTGCCTCTGGGTTCAAGCAATTCTCCTGCCTCAGCCTCCCAAGTAGCTGGGATTACAGGCGCCCTCTACC
ACGCCCGGCTAATTTTTTTATTTTTAGTAGAGATGGGGTTTCGCCATGTTGGCCAGGCTGGTCTCAAACT
TCTGACCTTAAATGATCCGCCCGCCTCGGCCTCCCAAAGTGCTAGGATTGCAGGCGTGAGCTACCGTGCC
CTGCCTTTTTATTATTATTATTTTTATTTAAAATTTTTATTTTTTGAGGCTGGAGTGCCGTGGTGCAATC
ATGTTTTACTACAGCCTCAACCTCCCAGGCTCAAGCAATCCTCCCACCTCAGCCTTCCAAGTAGCTGGGA
CCACAGGCGTGCACCACTGCACCCAGCTAATTATTTTTTTGTATTTTTTAGTAGAGATAGGGTTTTGCCA
AGTTGCCCAGGCTGGTCTTGAACTGCTGGGCTCAAGCAATCCTCCCCCCTTGACCTCCCAAAGTGCTGGC
ATTATAGGCGCGAGCCACTGCACCCAGCCTGCAAAGGGGGTTTCTTACAGTCAATTTTGTTCCCCTTTTC
TGGGACCTCAGTCTTTTTAGCTGAAAACCAATGGCATGGTCATTTCGAAGCAGCTGGATGAAGTATCCCA
AAATTCCACGTCTCCCCACACTGAGGCCCCTCCCTTGTGCCCTGAGGGTGGGGTTGTGGGTTAGGGGGTG
AGTGGCCCAGAAATTCCTGAGGGGATCACAGCAGATGGTCTAGAGCTTGGGAAGGGGCATTGGAGAGTCC
CTGAGCCCCAGCGCTGACCCCTACTCTCACTGCCACAGAGAAATTCAACACGTACGTGACCCTGAAAGTG
CAGAATGTCAAGAGCACGACCATCGCGGTGCGGGGCAGCCAGCCCAGCTGGGAGCAGGATTTCATGTTGT
GAGTCTGCAGCGTCTGGCATGACCTCACTCCCTGGCATAGCCCAGCACCTCGCTGGCCCCTCAGGGCAGA
GCTGGACACTGGGGAATCCCGGGTGACTCCCCTCTGTCCTCCCTCTTCCCCTGTCCACCTTATCACCTTC
ACCTCCAAAACACATCCCCAATCCAATCACTTCCCTTGGTCTCCGCAGCCACCAGCCAAGTCCCTGCTGC
CCGCCAGCCTCCGCCCTGGCCTCCTGTGCCATCCACCTCCTACAATCTGTTCTCCTCCAGGCAGCCAGGG
GGATATTTTTAAAACTGCAAATCACATCCTGTCGCTCCCATGTTCCAAACTTGTCCGTGGCTCATCGACC
TCACAATAAAACCCACAGCCTTCCCCGTGATTCCCACCAGGTTCTCGCTCTATCTCCCTCACCTGCCCCA
GCCACACCCACCTCTGCCCCTTTCTCCAACACTTCCCAGCTTGTTCTTGCCCCAGGGCCCTTGCACTTGC
TCTCCTGTCCCCAGCTGGCTCTTCTCCTTCAGGTCTCAGCTTGTGTCACCTCCTCAGTGAAGCCCTCCCT
GACCACTACCTCCAAGCTACAATTTCAGGGCATCATGCCTTCCCAGGCCTTGCCACCAATTTTGTTTGTT
TGTTTGTTTGTTTTGAGGCAGAGCCTCACTCTGTTGCCCAGGCTGGAGTACAGTGGCATAATCTCGGCTC
ACTTCAACCTCCACCTCCCAGGTTCAAGTGATCCTCCTGCCTCAGCCTCCCAACTAGCTGGGATTACAAG
CGTGTGCCACCACACCCGGCTAATTTTTGTGTTTTAAGTAGAGATGGTGTTTCACTATGGTGGCCAGGCT
AGTCTCAAACTCCTGGACTCAGGTGATCCGCCCACCTCAGCCTCCCAAGGTGCTGGGATTATAGATGTGA
GCCACCATGCCTGGCCAATGGAATTTTTTTCCTTTTTTTTTTGTTTTGTTTTGAGACAGAGCCTCACTCT
GTGGCCCAGGCTGGAGTGTAGTGGTGCAATCTCGGCTCACTGCAACCTCTGCCTCCTGGGTTCAAGCTAT
TCTCCTGCCTTAGCCTCCCGAGTAGCTGGGACTACATGTGCGTGCCACCATGCCCGGCTAATTTTTGTGT
ATTTAGTAAAGACCGGGTTTCACCATGTTGGCCAGGCTGGTCTTGAACTTTTAACCTCAGGTGATCCACC
CGCCTCGGCCTCCCAAAGTGCTGGGATTATAGGCATGAGCCAGGAGCCACCAATCCCAGCCAACAGATAT
ATATATCAGATATATATATACTTTTTTTTTTTTTTTTGAGACTGAGTTTTGCTTTTGTTGCCCAGGCTGG
AGTGCAATGGCGTGATCTCGGCTCACCGCAACCTCCGCCTCCTGGGTTCAAGCAATTCTCCTGCCTCAGC
CTCCCGAGTAGCTGGAATTACAGACATGCACCACCACGCCCGGCTAATTTTGTATTTTTAGTGGAGATGG
GGTTTCTCCATGTTGGTCAGGCTGGTCTTAAATTCCCAACCTCAGGTGATCCGCCCATCTCGGCCTCCCA
AAGTGCTGGGATTACAGGCGTGAGCCACCGTGGTTGGCCATATTTTAAAAATATATTTCTTAAGATAAAT
CTTTGGTAAACTGTAAATTCAGTGAATCTCACATTTCACCAAGTTTCCTGCTCCCCCGAATTCACATATG
TCTAGGGCGAGGCCTGCAGGGAAAGTGCTTTGAGGTGGGATCGTGTCCTCACATCATTAGATGGGGATGA
CACTGGGAAGGACAGAGGTGACCCGAATGTCCTTATCCTGTAGGTAAAGGGAGCTATGGAGGGTATTTGA
GCCTCTTCCTCACTTACCCAGCATGGTCAGTGTTGGAGGGCCCAAGGAAGGGCTCAGAAAGATGAAGGTG
CAGGCGATGTGTAACATCTGTCCCCACGCCATCCTTCCCGGCTTTGAATCCTGGTTCTTTTGTGATACCC
CGAGCTCTTAACTTCCCCCACTGGCCTCAGTTTCCCCTCTATAAAATGAGAACCACCATGTTACCCTACC
ACCTAGGACACTCCCAGGCCTAGGTTTGAATTGCAGTATGCTGTGTGTTCTCAGGCGAATCAACTAAACT
CCCTGGGCCCCCGTTTCCTCCCTCCTGCTCCCTCGACGCCCCCTCTCCTTTTTCTGCAGCGAGATTAACC
GTCTGGATTTGGGACTGACGGTGGAGGTGTGGAATAAGGGTCTCATCTGGGACACAATGGTGGGCACTGT
GTGGATCCCACTGAGGACCATCCGCCAGTCCAATGAGGTGAGTGGCGGGTGGCCTGAGGGTGAAGAAGGA
GGAGTGCCTTGCAGAAGCCCAGAGACCAGTGGACTAAACAAGATGGGCATTTATCTCCTATCCCCTGACA
ACATCAATGTAGACACTTGGGATTGCCACGAACCTTCCAAAGTCACTAGAGCCTATACACCTTCCATCTT
GCTCTGCCTCTCTCTCCATGTGGCTTCCACCTTGTGGCCCCAAATGGCTGCTTTAGGCTCACCATTGCAT
CTTCATTCCAACCACCGAAAGGTGCTGGTGGTGGGGGAGGGGGGTGGTTCATGCACATCTCATTGGCCAG
AACTTAGACATGTGGCCAACACCTTGCTGCAAGGGATTCTGGGAAATGTAGTCCTAGGCTGGAAAATGTA
GTCCTGGGCTGTGCTTCCCTAGGCACAGCTAAAAATGTATTACCTTGGAAGGAGAAAAGGGATAATATAG
GCAATTAGCATTTTGTGTCCCAGATGGGCAGCAGCAGGAAGCTTACACCAACTCATAAGAAGGTGCTAGG
TAACAACCCTGGCCCCTCAGCCTGTGCATCATTGTCAGCAGGTAGTAGTGATGTCTATTTTTTATTTAAT
TATTTATTTTTGAGACAGGGTCTCACTCTGTTGCCCAGGCTGGAGTGCATTGGCATGATCATGGCCCACT
GCAGCCTCGACTTCCCAGGCTCAAGTGATCCTCCCACCTCAGCCTCCTGAGTAGCTGGGACTGCAGGTGC
ATGCCACCAGGCCCCCCTTATTTTTTTTGTATTTTTAATAGAGACAGGGTTTTGCCATGTTGCCCAGGCT
GGTTTCGAACTCCTGGGCTCAAGCAATCCACCCACCTCAGCCTCCCAAAGTGCTGGTATTACAGGTGCGA
GCCACCATGCCAGGCCGTGGTCATGTCTATTCATCTGTCCCCACAGCCCAATCCCTGGCCTCCTAACCTC
AAGTCTCAACCTCTCCATGCTGACCCCAGGAAGGCCTTATTAGCTGGAGCTCTGATCTGTCCTCCCCTGT
CCATCTGCCTTGGGAGGCCCTTCAGATGGCACCTGCCCACTAGCCTCATCTCCCCCCACACTCTAACCTC
TACCCCTCCCAGATTCCACCCTCTGAACCTTTGCATAGTGTGTTCCCTGAGCTTCAAGTGTCCTCCTTTC
CATCACCTTCTAAATTCCCATGTGCCCACCTCCAGGAAGCCCTCTCTGACTTCTCTCTAGATCCTTTAGC
CCTCCTTCCTCCCTCTGGGCTATGACCTCATGGGGCTGGGGGTGCCTGTGTCCAGCCCTGTTTCCAAGGG
CTATGGGCTCTCAGGAGCCAGGGTGATGATGGCTGGGCCTCTTGGGGACCCTGGCATCACCCAATATGAA
CCAGGCATAGAGGAGGGACTGGGGACTGTTTTTTGTTTGTTCGTTTTTGTCATTTTATTTTATTTTATTT
TATTTTATTTTATTTTATTTTATTTTATTTTATTTTACTGTGAGATGGAGTCTCACTCTGTCACCCAGGC
TGGAGTGCAATGGCACAATCTTGGCTCACTGCAACCTCTGCCTCCCTGGCTTAAGTGATTCTCCTGCCTC
AACCTCCTGAGTAGCTGGGATTACAGGGGCCTGCCACCACACCCAACTAATTTTTGCATTTTTAGTAGAG
ATGGGGTTTCGCCATGTTGGCCAGGCTGGTCTTGAACTCCTGACCTCAAGTGATCCGCCCACCTTGGCCT
CGCAAAGTGCTGGGATTACAGGTGTGAGTCACCGCACCTGGCCAGTGAGTGTTTGTTTTTGAATGAATGA
ATGCAGAGGGACGAAATGGTTGCAACAATCTAGGTAAGCGAGTCTAAAAGGAAGCCTTGGCTGGGCATGG
TGGCTCACACCTGTAATCCCAGCACTTTGGGAGGCTGAGGCAGGTGGATTGCCTGAGCTCAGCAGACCAG
CCTGGGCAACATGGTGAAACCCCGTCTCTACTCAAAAATACAAAAAATTAGCCAGGAGTGCTGGCACGTA
CCTGTAATCCCAGCAGCTCAGGAGGCTGAGTTAGGAGAATCACTTGAGCCTGGGAAGCGGAGGTTGCAGT
GAGCTGAGATCACGCCACTGCACTCCAGCCTGGGTGATAGAGAGACTCTGTCTCAAAAAAATTAAATTAG
GCCGGGCATGGTGGCTCATGCCTGTAATCCCAGCACTTTGGGAGGCCGAGATGGGTGGATCACGAGGTCA
GGAGATTGAGACCATCCTGGCTAACACGGTGAAACCCCGTCTCTACTAAATATACAAAAAATCAGCCAGG
CGTGGTGGCGGGTGCCTGTAGTCCCAGCTACTCGGGAAGCTGAGGCAGGAGAATGGCGTGAACCCAGGAG
GCAGAGCTTGCAGTGAGCCGAGATCGCGCCACTGCACTCCAGCCTGGGCGACAGAGCGAGACTCTGTCTC
AAAAAAAAAAAAAAAAGAAAAGAAAAGAAAAAAGAGAGAGAAAGGAAGGAAGGAAAAGAAAGGAATAAAG
GAAGGAAGGGAGGAAGGGAAAGAAAGAAAGGAAGGAAGCAGGGAGGGAGGGAGGGAAGGAAGGAAGGGAA
GGAAGGAAGGACGGAAGGAAAGATAGAGAGGGGACATGACTTTGGTTTTGGGGTAGGGGTGAGGGGGATG
TCTCATGCGAGAGTAGGGGCCCTGGGGACTAGTGACCAGCAGATTCCTGACCCACACACCTCCTCCCCCA
ACCCCCAGGAGGGCCCTGGAGAGTGGCTGACGCTGGACTCCCAGGTCATCATGGCAGACAGTGAGATCTG
TGGCACCAAGGACCCCACCTTCCACCGCATCCTCCTGGACACGCGCTTTGAGCTACCCTTAGGTGAGTAC
AGGGGCCCGGGACCTTCACCCAGCCCCAGCCCCAGTTGTGGACTCACTCAGCTATTCCAGGAGAGTGAAC
AGGCCTGGGTAGGTCTAGCTGACCCTTCAGGCTGAGAATGAGGGTCCTTCGCCCCGGAGGAGAGAGAGTT
TCTTGGGTTGCAGAGAACAGGGATGTGAACCTGCTGGGTGCGGGCCATGACCTGGGCCCCGTAGCTTCCC
AGTGAGCTGCAAGTGCTTCACGACCGAGGGCGTTCAGGGTTGAGCTATCCTGGCTGTTCCTTGGTGATGC
CTGAGACTGCAACAGTGAACTGGGGATAGGAGGAGAGACAGACAGAGACAGAGAGACAAAGAGAGACAGA
GAGAGACATGTAGACAGAGGCTGAGAACAGATAGTCCCCTCTTCCTTTCCTGGAAGCACCTTGAGTCTCT
CCACCTCATTCTTCTAAGAACCCATGGCTGGCCAGGCATAGCTTCTCATGTCTGTAATCCCAGCACTTTG
GGAGGCCAAGGGGGGAGGATCATTTGAGGCCAGGAGTTTGAGACCAGCCTGGGCCATCTAGTGAGATCCC
ATCTCTATGAAAAAGTTAAAAATTCACCAGGCATGGTGCTGGGTTTCTGTAGTCCCAACTATTTGGGAGG
CTGAGGCAAGAGGATGGCTCAAGCCCAGGAGTTTGAGGCTTCAGTGAGTTGTGATTGTGCCACTGCACTC
CAGCCTGGGTGACAGAGCGAGACCCTGTCTCTAAAAAAATAAAAATAAAAACAATGGGCTGGGCGTGGTG
GCTCATGCCTATAATCCCAGCACTTTGAGAGGCCGAGGCAGATGGATCACCTGATGTCAGGAGTTTGAGA
GCAGCCTGGCCAACATGGAGAAACCCTGTCTCTACTCAAAATACAAAAAAATTAACTGGGCATGGTGGTG
CACACCTGTAATCCCAGCTACTCGGGAGGCTGAGGCAGGAGAATCGCTTGAACCCAGGAGGTGGAGGTTG
CAGTGAACTGAGATTGTACCACTTCACTCCAGCCTGGGCAACAGAGTGAGACTCCGTCTCAAAAAACAAT
TTAAAAATTAAAAAATTAAATAAAAACAATGGCTTCCCCACCCTCGTAAAGGCTAGGAACCATCTCCTGT
CATCCTCCAACTATTCTCCATTCAGCTCTGGGAAGAGGGGCGTCAGAGGCACCACGGTACCTCCCCTTCA
TCTCCATCCACCCTCCTGAGACCGCAGACCAACCCACCCCTACTTCTGAGGCTTTGCCAAAGACCCCAGA
AGCCAGGGCCCGGGTGGAGCTCAGTAGGCCTGGAGGATGGAGGCTGAGCGGCAGGGAGGGTCTTCCAGGC
TCACAGACAGGGCTCTGCTTGCAGACATTCCTGAAGAGGAGGCTCGCTACTGGGCCAAGAAGCTGGAGCA
GCTCAATGCTATGCGGGACCAGGATGAAGTGAGTGTTGGGGTGGGAGGGGACTGTTTCTTCCTTGTTTCT
GTCTCTGTCTCCCCATGATTTTCACTTACAGATGCTGTTTCTCTCTAAGCTTGTCTTTCCCTGCATCTCT
GTTTCTAACTCTTTGTAACCTGACTGCATCCATGACCCATGTGGTCCAGTCTCTAGACAATGATGGGGTC
AAGAAACACACAGAGGCCAGGCGCAGTGGCTCACGCCTGTAATCCCAGCACTTTGAGAGGCGAAGGTGGG
CAGATCACCTGAGGTCAGGAGTTCAAGACCAGCCTGGTCAACACAGTGAAACCCCGTCTCTACTAAAAAA
AAAAAAAAAAAAAAAAAATTAGCCAGGCTTAGTGGCTCACACCTGTAATCCCAGCAATTTGGGAGGCTGT
GGCGGGCAGATTATTTGAGGCCAGGAGTTCGAGACCAGCCTGGCCAACACGGTGAAACCCCGTCTCTACT
AAAAGTACAAAAAATTAGCTAGGCATGGTGGCGGGCGCCTGTCATTCCAGCTCCTTGGGAGGCTGAGGTG
GGAGAATCACTTGAATCTGGGAGGCAGAAGTTGCAGTGAGCCGAGATCACGCTGCTGCACTCCAGCCTGG
GCAACAGAGCAAGGCTCTGTCTCAAAATAATAATATTGATAATAATAATAAAATAAAAAATTTTACTTTT
CATTTGGCGGATACATGTGCAGGTTTGTTACACGAATATATTGCATGATGCTAAGGTCTGGAATTCCATG
GCATTACTCAAATTGTAAACACAGTACCTAACGGGTAGTTTTTTCACTCTTGTCCTGCTCCCTCCTTCCT
CCCTTCTGGAGTCCCCAATGTCTACTGTTCCCATCTTTTTTTTTTTTTCTTTTTTGAGACGGAGTCTCGC
TGTGTCACCAGGCTGGAGTGCAGTGGCACGATCTTGGCTCACTGCAACCTCCACCTCCCGGGTTCAAGTG
ATTCTCCTGCCTCGGCCTCCCGGGTAACTGGGACTACAGGCATGCGCCACCATACCTGGCTAATTTTTGT
ATTTTTAGTAGAGACGGGGTTTCACCGTTTAGCTAGGATGGTCTCGATCTCTTGACCTCGTGATCTGCCC
GCTTCGGCCTCCCAAAGTGCTGGGATTACAGGTGTGAGTCACTGCACCTGGCCCCTAGTCTTTTTTCTAT
CTCTGTTTCTTACTTTTTCTGTCTCTGTCTCTGTCTCTGTCTTTCTCTCTCTCTCTCTCTCTCTCTCTCT
CTCGTGTGTGTGTGTGTCTTTCTCTCCTTTTCTCTCTTACTCCCTGTATCCTTCTCATAGAGAGGGAGAG
AATTTGGGAGGTAGAGGGAAGTCGGGATGGGACAGAACAGAGGACTATCCTGGTCCCCTGGCCTTTGGCC
CCTCTGAGAGAAGCCCTTTCTCCTTCCTCCACCCCTTCAGTATTCGTTCCAAGATGAGCAAGACAAGCCT
CTGCCTGTCCCCAGCAACCAGTGCTGTAAGTACCTCTTGATTCTTCCTTGGGTATCAGCTGAAATCCTCA
TCCCTCCCCTACCCCATGACTGTCCTGTCTAAAGGCTCCTCCAGTCCGGAGATACAAGTCCTGAGAGTGC
AGTGTCCACTTACAGTGTATTAGAAATCAGCGTTCTGAGGCTGGGCATGGTGGCTCACCCCTGTAATCCC
AGCACTTTGGGAAGCCGAGGTCAGGAGTTCGAGACCAGCCTGGCCAACAGGGCAAAACCCCATCTCTACT
ATAAATACAAAAAAAATGAACAGGCATGGTGGTGGGTGCCTGTAATCCCAGCTACTCAGAAGGCTGAGGC
AGGAGAATTGCTTGAACCCAGGAGGCAGAATCTGCAGTGGGCCAAGATCATGCCACTGTACTCCAGCCTG
GGTGACAGAGCAAGACTCTGACTCAAAAAAAGAAGGAAGGAAAGAAAGAGAAAGAAAGAGAGAAAGAGAG
AGAGAGAAAGAAAGAAAGGAAGGAAGGAGAGGAGAGGAAAGAAAGAAAGAAAAAGAAAGAAAAGAAAAGA
GAAAAGAAAAGAAAGGAAGGGAAGGAAAGAAAGGAAAGGAAGGAAAGGAAAGAGAAGAGTTCTGAGACTT
GGGCTTGATGAAAGACAAGAAGGAAGAAAGGAAGGAAGGAAGGAGAGTTCTGAGACTTGGGCTTGATTCC
CACCTTGCTATGTGGTCATGGGCAGGCCAGTGTCCCTCTTCGATATTTAGGGGCCCTCTCTTTGAAATAG
CGCATCTGACAGCCCCCTCACTCTGAGGGCTGTTGGAAGGATCTGGGACATCTAGAGACATGAAAAACAC
TTCGTAGATGCTTCACTTCCATTCATCAGATCCTGGATGAGGGCGTATAACTCATTCCACAAACTATTCA
TGGGGCCAGCACAAACCCCGAGCAGCCCACGTCAGATGGTGCAGACAGACACAGGCAGAAGATCCTGCCA
GCACATGTACAATGGGCGCACGTACAACAGACGAAGGCTGTTTCACTAACCCATTTTCTTAGATGGAGAA
ACTGAGGCCCAAGAGGAGATGTGATTTACCCAAGGCCACACACCCGGCAACTGGCAGAGCCAGGATTTGA
ACCCAGGTCTGAGGGACTCCAACTTTGCTTCCCTTTCCACTGCACCTTGTCTGTTAATGATGATGATGAT
CACGTTCCTCTTCCTATTTCCATCAAGGCTGCAGTTGTCAAGATTTTTTATCTTCCTGGGCCTTGTGGAC
ACTCAGATGCATCCAGACCAGGTCTTTTCATGGATGGAGGAGCTGTCTCCCTCCTTAAAATGACTTCCCA
TTGCTCTGTGGTGGAGGGGAAGTAGCAAATCATATAGCCTCAGGGCAGAGCTCCCACCTGCTGACACCAA
AACCCCACTGCACTCTTTAGCCTCCTTCTCCTTTCCCATTTACTTTCTTTTCTTCTTCTCTTTTTTTTTT
TCTTGGAGATAGAGTCTCCCTCTGTCACCCAGACTGGACTGCAATAGTACTATCTTGGCTCACTGCAGCC
TCTGCCTCCCGGATTTGAGTGATTCTTGTGCCTCAGCCTCTTGAGCAGTGGGGATTACAGGCACATGCCA
CCACGCCCAGCTAATTTTTGTATTTTTAGTAGAGATGGGGTTTTGCCATGTTGGCCAGGCTGGTCTCAAA
TTTTTTACCTCATGTGATCTGCCCACCTCAGCTTCCCAAAGTGCTGGGATTACAGGTGTGAGCCACCATG
CCTGGCACCTTTTCCATTTGCTTTTCCATCTTCCTTTTCTCCCCTCTCACAGCTCTCCCTGTATCTGTGT
CTCCCTCACCATGTGTCCAGGAGGGTGGTAATGGAACCGGGGTTGAGGAGGGCTTGGGATACTCCAGCTG
GGGTACCCTGTTTCCACACTAACTTGGGGAGTGATATTGAGAAAGTTCCTTGGCTTCCCTGGCCAGGGAG
GGGTCATCACTGCCTGGAAGGAATTACAAACCCCCGCTTGGTGAATGGGGTCATTGAGAGGTTGCAATGG
GGGCGGGGCCAGGCAGAGTGGAGGCAGGAAGGCAATGGCAGAGAGCCCCTGGGCAGATGGACTAGAGCTA
GGGGCAGGTTCATTTTGGGAAGTGGAGACCACCACTCAGTATTTACCCACAAGTCCTCCCTTGGAGTATT
TTGTCACCCTATTTCTCAGAGAAAGCAGCCAATGGTCAGAGGAGCCAGGATTCTGGTCCAGGACATTTTT
CTGAAGTGCCAGGTACTGCACGTGATAGGCAAAGGTGGTTCTCGCCCTGGCCCTGATGCGCAGAGGCTCT
GTGACTTTGGGCTGCTCACTCCATCCCTCTGAGCCCCATAGTTTCACCCATAAAATGAGTAGAATGATAA
AATCCACCCAACAGGGGTGTTTCAAACCTTAGGGTTTCCAATTTAGTGTGTCTTTCCTTATCCAAGAGCT
GGGAGAGGAAAAGGGTGGAGACTGGGAGACTGCTAACCCTAGACATCATAGTCAGAAAACCCTGGGCCCA
AATACCCACTTTGTTACTTCCTGACTGAGCCTTTCCTCTCATTGAAAACTGAGCCAGGCACAGTGGCTCA
TGCCTGTAAATCCCAGCTACTCGGCCTCACTTGAGACCACGTGTTTGAGACCAGCCTGACCAACATAGCA
AGATCCATCTCCACACAAAAAAACAAAAAGAAAACCACCAAGAACCAAAACATTAGCTGAACATGGTGGT
GTGTGCCTGTAATCTCAGTTACTCAGGAGGCTGAGGTGGGAGGATCACTTGAGCTTGGAAATTGGAGGCT
GCAGTAAGCCGTGATTGCACCACTCTACTCCAGGCTGGATGACAGAGTGAGACCGTGTTTCAAAAAAAAA
ATTAAAGAAAAGAAAAAAATAAACAAACAGGGATATCCCACCTTCCTCCCAAGGTGGAGAGCAGAGACAC
AGCAAGTGTCAGCAATGAAGGTGATACTCAGGGAAGGTACTCAGGACAAGAGGGACTTTTGGGGTGGGGG
GAGCAGTTGGGGAGAGTGAGGACAGCAGGGGAGCCCCCTTCCTACACCCACCCCTCTGTCTGCTCCCTTT
TATTATTTCTGCCCCTTCTCTTTGTAGGCAACTGGAATTATTTTGGCTGGGGTGAGCAGCACAGTAAGTA
CTCAGCCTGATTGATGTTCTGGGAGTTACATGGGAAGGTGGGCACCTTGGTGATCCCAAGGCCCCTCCCC
ACTACCACACAGGGAAGCAAGGAGCCTGTGACGAGCACGTGGGGGCCAGAGGGCTCAGCAAGACCCCTTT
CAAGCTTTCCTGTACCCTGTTCATTAGTGCCTGTCTTGGGAGGCCTGCCATCTGCTGGGGAGTGGGCACC
GTCATTGGGGTGGGGTGGGGGGGGTTAGCCTGGAGGAGTGGACATTTAGACCAACAACCTGCTTGTCTCC
AGCTGGGCACTGTGACCCCCGCACCCCACTCCGCCGTATAACCTCACCTCCTCTCCTGTAGCCCCTGCCT
TTTGGTCTTCCTATCTTCCTTCTCCACTTGGAAATGTGTCCAGTGTCACTCAGAGGGTAGTGGGTGGGCG
GGGTCTCTAACAGAACGGCAGGCCAGGGACCTCAGGGAGATGCACACAGGGACCTCAGCACCCCCCAGGT
CCACCCACACTCACACCTCACCTTCCACCTTCATTTGCTCTCCCAGGGAATAAGGCAAGCCACCCCCTCC
CTAAATTAAATGTCCATTTTCCTTTTCCTTTTTTTTTTTTTTTTTGAGACGGAGTCTCACTCTATCACCC
AGGCTGGAGGGCAGTGGCACGATCTCAGCTCACCGCAAGCTCTGCCTCCAGGGTTCACGCCATTCTCCTG
CCTCAGCCTCCCGAGTAGCTGGGACTAAAGGCGTCCGCCAACACGCCCGGCTAATTTTTTGTATTTTTAG
TAGAGACGGGGTTTCACCGTGTTAGCCAGGAGGGTCTCGATCTCCTGACCTCGTGATCCACCTGCCTTGG
CCTCCCAAAGTGCTGGGATTACAGGCATGAGCCATGGCACCTGGCCCTAATTAAATGTCCGTTTTTCTAC
CTAACAGCGGTTCCCAACCTTTTTGACACAAGGGACCAGTTTCATGGAAGACAATTTTTTTATGGACTGG
GGTTGGGGGATGGTTTCTGGATGATTCAAGTTCACTGCATTTGTTGTGCACTTTATTTTTATTATTATTA
TTATTACATTATAGTATGTAATGAAATAATGATACAACTCATCATCATGTAGAAGCAGTGGGAGCCCTGA
ACTTGTTTTCCTGCAATTAGACAGTCCCATCTTAGGGTGATGGGGGACAGTGACAGATCATCAGGCATTA
GATTCTCATAAGGAGCACACAACCTAGATCCCGCACATGCACAGTTCACAGTAGGGTTCACGCTCCCGTG
AGAATCTTTTTTTTTTTTTGAAACAGAGTCTTGCTCTGTTGCCCAGGCTGGATGGAGTACAGTGGCGCAA
TCTCGCCTCACTGCAACCTCTGCCTCCGAGTTCAAGTGATTCTCGTACCTGAGCCTCCCAAGTAGCTGGG
ATTACAGGCGCGTGCCACCATGCCCAGCTAATTTTTTGTATTTTTAGTAGAGATGGGGTTTCACCATGTT
GGCCAGGCTGGTTGTGAACTCCTGACTTCAGGTGATCCACCTGCCTTGGCCTCCCAAAGGGCTGGGATTA
CAAGTTTGAGCCACTGCGCCCGGCCTTCTATGAGAATCTAATGCTGCCGCTGATCTGACAGGAGGTGGAA
CTCAGGCGGTAATGTGAGTGATGGGGAGCAACTGTAAATACAGATGAAACTTCACTCGCTCACCCAACCG
CTGCCCACCTCCTGCTGTGCGCCCCAGCTCCTAACAGGCCACAGACCAGATCCATGCCCTAAAGAGTTGC
TCCCTTTCTCCCATACCCCGCCTCTTCTAGAGAAATCCAGTCCTCTATTCTTTCTTTCCTTCTTTCTTTT
TTCTTTTTTTTGAGATGGAGTCTTGCTCTGTCACCCAGACTGGAGTATAGTGGTGCGATCACTGCTCACT
GCAACCTCCACCTCCCGGGTTCAAGCAGTTCTCTTGCCTCAGCCTCCCGAGTAGTTGGGATTACAGGTGC
ACGCCACTATGCCTGGTTAATTTTTGTATTTTTAGTAGAGATGGGGTTTCACCATGTTGGCCAGGCTGGT
CTTGAACTCCTGACCTCAAGCAATCCTCCCGCCTTGGCCTCCCAAAGTGCTGGGATTATAGGTGTGAGCT
ACCATGCCTGGCCAGTCCTCTATTTTGAGGATAAATCTGCCTCCTTCTCTCTCCTTCAACTGGGGAGTCC
TTCACTCCTTCCTCATATCCTGGTGGACAAACCTAACTCCAGGACCAGTGAAACTGTTGACAAACACTTT
GGTCCACCACAATTTATCAAAAACAAGTTGGTTAAAAATCAACTTAGGCCAGCCACACTGGCTCACACCT
GTAATTCCAACACTTTGGGAGGCTGAGGCGGGAGGGACGCTTGAGCTCCAAAGTCTGAAATCAGCCTGGG
CAACAGAGCAAGACTCCATCTCTACAAAAAAAAAAAAAAAGGGCCGGGTGTGGTGGCACGCCCCTGTAGT
CCCAGCTATTTGGGAGGCTGAGGTGGGAGGGTTGTTTGAGCCCAGGAGGTTGGGACTGCAGTGAGCCATG
ATTGTGCTACTAGACTTTAGCCTAGGCAACAGTGTGAGACCCTGTCTCTAAAAAATAAATAGATTAAAAT
AAATAAATAAAATCAATTTTGCCAAAATGAATGCTTGGCAGACAGGGCAAGCTCGATAACAACCAACTTG
CCACAAACGAACAGTACGGTCAAAAGCAAAGATAGACCAGGCGTGGCAGCTCACACCATAATCCCAGCAC
TTTGGGAGGCCGAGGTAGGCAGATCACTTGAGGTCAGGAGTTCGAGACCAGCCTGGCCAACGTGACGAAA
CCCCATCTCTACTAAAAATACAAAAATTAGCCAGGCATGGTGGTGCATGCCTTTAATCCCAGCTACTCGG
GAGGCTGAGGCAGGAGAATCATTTGAACCTGGGAGGTGGAGGCTGCAGTGAGCCGAGATCGTACCATTGC
ACTCCAGCCTGGGTGACAGAGCGAGACTCTGTCTCAAAAAAAAAAAAAACCCAAAAACCAACAAACAAAC
AAAACAAAACACCAATGCAGCTAAAGCTGATAGATAATTGGGTCAAAAAGGAAAGACAATTGAACCGAAA
ACAGACCGTTGGGTTAAAACAAAAGTGGTTGAAAATAGCCAGAAGCAGGCAATTTAGTCAAGAGCTACTT
AGTAGAAAGCAGAGCCTGGGCAAAAGCAAAAAGAATCCAACTCAAAGTAATTTACCCTTCATAAGCAGAT
AATTTGTCAGGAAAAAAAAAAACTGGGTAAACATGTGCACAGCCTCATGCAATTTGGCTGGAAACATCTC
AGCCCGGTCAAGGACAATTGGATAGAAAGAATGCTGGCCGGCCGAGGTGGCTCACGGCTAATCCCAGCAC
TGTGGGAAGCTGAGGCGGGAGGATCGCTTGAGCCCAGGAGTTCAAGATCAGCCTGGGCAATATAGGGAGA
TCCCATCTCTACAAAATAATAAATAAATAAAATTAGCTGGGTGTGGTGGTGTGTGCCTGTGGTCCCAGCT
ACTGTGGAGGCTGAGATGCGAGGATCTCTTGAGCCCAGGAGGTCGAGGTTTCAGTGAGTTTTGATCGCGC
CACTGCGCTCCAGCCCAAGTGAGAGAGACCCTGTCTAAATGAATGGATGAATACAGCAAAAGTCTGAAGG
TTTCACTCATTGCTCTGAGATTTTGGGGTCTGGGGGTTTTTTCATTTTTTTACATCCTCTTCCCCTTACC
CGTACTTCTCATGTTTTCTTCATGGACTACTTTTTCTTTTTTTCTGGATACAGGGTCTCATTCTGACACC
CAGACTGCAGTGCAGAGGCGTAATCATAGCTCACTGCTACCTCGACCTCCTGGGCTCCAGCAATGCTCCT
GCCATACCCTCCCGAATAGCTGGGACTACAGGCGCCCACCACCACGCCCAGCTAATTTTTAAATTTTTTT
AAAGAGATGGGGCCTTACTATGTTGCCCAGGCTGATCTTGAACTCCTGGGCTCAAGTGATCCTCCTGCCT
TGGCCTCCCAAAGTGCTGGGATTACAGGCATGAGCCACTTTGCCTGGCCCATGGACTAGGTTTTACTTTA
CTTTTATTTTATTTTATTTTTTGGAGACAGGGTCTTGTGTCACCCAGCCCCTGGAGTGCAGAGGTGTGAT
CTTGGCTCACAGCAATCTCCACCTCCTGGGTTCCAGAGATTCTCCTGCCTCAGCCTCCCAAGGAGCTGGG
ATTACAGGCACATGCCACCACACCTGGCTAATTTTTGTATTTTTAGTAGAGACAGGGTCTTGGTATGTTG
CCCAGGCTGGTCTTGAACTCCTGGCCTCAAGTAATCTGCCCGCCTCCGCCTCCCAAAGTGCTGGGATTAC
AGGTGTGAGCCACTGTGCCCGGCCTTTGCTTTACTTTTAAAATAAAGATAACTTTATCATTCTTTTTAGT
TAAAAAATTAAAAAGATTGCATTATTGAAATCTTTTTTTTCAGAAGGTACAGAAAAGAATAAAGAAGAAA
ATGTACAATCCCCAATTATGCCACCACCCAGACATGTTGCTTTTAGAATTGGAATTTTTTTTTTTAGGAT
TTAGAAAACAAACTGTTCTTTTTTCTAAGGAAACCAAACTAAACCAAAATATCTTTTAGTTCTTATTAAA
CATGAAGTTTGGCAACTCTACCACATTCCATTTTAGCAATAGATTCCAAGTGTGTGGTGGCTTTGCCAAG
CTAAGAATCTAATCCTTTTTGAAAAAGACAGAACCATGTCTCAGCATCTTCTTGGTTCTTGCATATTTGT
TAAGTTCAGGTCTGGGGTCGTCACCCTCAGATATCAGGAAATCTCTAAGGGTTAAAGAGCGTTCCGTCCA
CTTTGAACTCCTGATCTTCTTCCAACTTCCCCCTGACTGTCTGTGTGACTCCTGTTATGGGAAACTAACT
GCTCAGTGGAAAAAAATCCCATGTCATTTTCAGAGATCCTGACCTTCAGCAAAGTTGTTCTTCACATTTG
AGCTGAAGGATGCCCTCTACTCTGTCCCTGTTTTCTTCACATTGTAGGTCCTACCCTAGCCCCTAGACTT
TAGGTCCAATTTGACCCACATAAAGCAAAGCAGGGTTTTCACCTCCCTTGTTCTGAAGCCTATATCTCTA
TTAACATGACCCACAATTCTTTATCCATACTAGTAAAAAATTCTCTTCTTCGGTAGCGGCACCGTAAGTC
TGGGAAATTGTATTGGCTCCCATCATGTGCACTCTCTCTTCCTCACCCTCTTCCCATACCGCCTCTCTCT
TCCAGACGATGACCCCGACAGTGCAGTGGATGATCGTGACAGTGACTACCGCAGTGAAACGAGCAACAGC
ATCCCGCCGCCCTATTATACTACGTCACAACCCAACGCCTCAGTCCACCAATATTCTGTTCGCCCACCAC
CCCTGGGCTCCCGGGAGTCCTACAGTGACTCCATGCACAGTTACGAGGAGTTCTCTGAGCCACAAGCCCT
CAGGTAGGTGCTGCGGAGTGCAGCATGCATGTGGGTCCTGCTGTGTCTCCTGGAGTGGAGAGAGGGGTAC
GGGGCTGGACAGAGCTGGAGGTGGAGTTCCAGAAGGGCAGAATTCACCCATCCAGGAGGGCAGAATTCAC
ATTTTCTTGTGGGAATTAACCATTTTTTTCTTTTTTTTTTTCTTTTCTTTTTTTTTTAGACAGAGTCTTG
CTCTGTCACCCAGGCTGGAGTGCAGTGGCACCATTTCGGCTCACTGCAACCTCCACCACCCAGACTCAAG
CGATTCTCCTGCCTCAGCCTCCCCAGTAGCTGGGACTACAGGCACAGGCCGCCACACCAAGCTAATTTAT
TTTTATTTTTTGTGGAGATGGGGGTCTTGCTCCATTGCCTGGGCTAGTCTTGAACCCCTTTTTATTCCTA
TAGCATCCTCGAGGCTGGCCAGGGAAGAGGGTCTCAGGCGCTAAGATTGGAGGAGGTATCTAATCGGGAA
ATGATAAGGTTCGTTGAATGACTAAGTGAATGGTTAGAAGAGGGCTGAGTGGTGGCCCCTATGTCCCCCT
GCCTGTATTTAGACAGAGACAGATATTGCTGTTGACGTTGAGCACGATCCTGGGTTCACATAAGCAGCAC
ATAACAGAGGGCCTGACCTGGCCAGACAGTGGGGGGCATTTGCACAAGTAAGTGATGTTTCAGTGGGTAC
CAGAGGGTGGGTAGGAGTTTGCTAGATAAAGCAGACAAGGAAGAGACCACGGCAAGTGCACAGGTTTATG
GTGTTGGAGAGACAACGAGGTGGCCAGCGTGGCCGGAATGGAGGAGGCGGGGAGAAGTGGAGGGAGGTAT
GCAGGGGGAGTTTGGAGCGTATGGGCCACACAGGGCTAGCACAGATTTTATTCCAAGGCACTAGGGAGCC
ATGGTAGGATTTAGAGCAGGGGAGGGAGGTGATTGGATTTACAATTCCCTGTGGCTTCCAAGGGAAGCAG
GAACTGTGGAGACAGCATCTGGTAGAGACAGCAGAGGCTGGCCTGGGTTGGGGCCCGGGGTACGGGGAAG
TGGATGCATTTCCCATACTTTTTCCAGGTGGAGGACAGGAGACCTGCTGCGGGGCTGTGGTGATGGCGCT
GACCCTGAGTTCCCAGCACAGTCAATTGCCTGTTCCCCACAGACCGCAAATGCCACAAAAGTGGGAATTT
TTTTTTTTTTTTTTTTTTTTTTGAGACGGAATTTCGCTCTTGTTGCCCAGGCTGGAGTGCAATGGCATGA
TCCCAGCTCACTGCAACCTCCACCTCCCGGGTTCAAGTGATTCTTCTACCTCAGCCTCCCGAGTAGCTGG
GATTACAGGCATACGCCACCATGCGCAGCTAATTTTTGTATTTTTGGTAGAGACAGGGTTTCACCATGTT
GGCCAGGCTGGTCTTGAACTCCTGACCTCAGGTGATCTGCCCACCTCAGCCTCCCAAAGTGCTGGGATTA
CAGGCGTGAGCCACCGCACCTGGCCAAAAGCGGGGATTTTTGTCTTTTCTGATCATGGCTGTGTTTTCCA
GCCCCTAGAGCAGGACCTGGCACATCGTAGGTGCTCAGTCGGTGATGAGTCAATGCCTGTGTGACTGGGG
CTCAGGTGGGCAGTGACTCGGTACCCCAGTCCCTGAACCCTGTTCCATCCCCACAGCCCCACGGGTAGCA
GCCGCTATGCCTCTTCCGGGGAGCTGAGCCAGGGAAGCTCTCAGCTGAGCGAGGACTTCGACCCTGACGA
GCACAGCCTGCAGGGCTCCGACATGGAGGATGAGCGGGACCGGGACTCCTACCACTCCTGCCACAGCTCG
GTCAGCTACCACAAAGACTCGCCTCGCTGGGACCAGGATGAGGAAGAGCTGGAGGAGGACCTGGAGGACT
TCCTGGAGGAGGAGGAGCTGCCTGAAGATGAGGAGGAGCTGGAGGAGGAGGAGGAGGAGGTGCCTGACGA
TTTGGGCAGCTATGCCCAGCGTGAAGACGTAGCTGTGGCTGAGCCCAAAGACTTCAAACGCATCAGCCTC
CCGCCAGCTGCCCCAGGGAAGGAGGACAAGGCCCCAGTGGCACCCACCGAGGCCCCCGACATGGCCAAGG
TGGCCCCCAAGCCAGCCACGCCCGACAAGGTGCCTGCAGCTGAGCAGATCCCTGAGGCTGAGCCACCCAA
GGACGAGGAGAGGTAACAGGACGGGGCCAAGGGGCCAGAGGGGCTGCCACAAGGTCACAGGGTCAGCAGG
GCTATGATGGGGTCAGCGTGGACCTGGGAAGTTGCTGGGGCTGAAGTTGTCACAGGCTCTTGGGTTTCTT
AAAGGGATCAGAGGTGACAGGGCAGAGTAGGGAGGTCAAAGAGGGTTCAAGGGGCCGTGAAGGAGACTTA
GAGTTTGTGATCTTAGTGGTTGCTGGGGTCGGAGAGGTCAAGATCCTGGGCAAATGGGGGGCCAGAGGGG
CTGGGCTGGTGAAAAGGAAGCCGAGGAACAGTGAAGTGTCACAGAAGCATTGGGTGTGTGGGAAGGGGTG
AGGGGCACTGAGGTGGGCCAGGGGACTCACGTGGGTCCTAATGTCTCTGAGGGGTCAGAGATACCATGGG
GAGCTATGCATCACTTGCACAGCTGGAGGGTCAAGGGAAGTTCAGGGGTCACGGAGAGCCAGCCCAGAGC
CTCATAGCGTTCCCTGCCTCAGTTTCAGGCCGAGAGAGGATGAGGAAGGCCAGGAGGGGCAGGACTCCAT
GTCCAGGGCCAAGGCCAACTGGCTGCGTGCCTTCAACAAGGTGCGGATGCAGCTGCAGGAGGTGAGTGAC
ACGCCCAGCCATGCCCCTGCCACACTTAGCCAGGCCACGCCCAATGGCAGGCCCACACATGTTTTGGCCA
CACCCACACCTATATTGGCCACGCCCAACACCCCACCCGTACCCAAGCCTAGCCTGGGCATGCCCAGTGC
AGTGCCCACACTGTGCCCATGCCCCAGCCACTCGAGCCCTTCCCTGGCCCCACCCTCATTCAGCCTGGCC
TCACCCAATGTCACCGATGCCCCACCCACTCTGACCCTTCCTTAGCCACATACACACACAGCCCAGGGTA
TCCCCATGGCCCGTCCACACCAGCCTGGCTATGCTAACTCCCAAACCATGCACCAAGTCTGGCCCCACAC
ATCCCCAGCCCAGCCTCCTTCCCAATGTCAGCCTGGGGTTTCTAGATCTAGAGGGAGGAGAGAAAACTAG
GGCTGAAACTGGGCTGAAGTCATGGTTAGGGATCAAGGTCAGGGTTAGTTCAGCCATCAAAGATCAGAGG
TCAGGGTTAGAATTGGTGTTAGGCAAAGGGGTCAGTGCTTAGGGCCAAATTGGAGGTCATTGTCAGGGTT
AGGGAAAGGTCTGGGGCCGGCTCAGTAGGGGAGTAGGAATCAAGGGAAGCAATCAGCATCAGGGGCAAGC
TGACTGTTTAGATGGGCAGCAGGGTTGAGGCCATTCGTTTATTCCTTCATTCAGCTCACCCTGGGCTGGG
GACAGCTGTGTTGCTCTTCAGAGTTACCCTCCAAACTGCAGTGGGATTGGCAGGAGGAGATTCTAAACAG
ACTCCCAAAGGTTTAGACCATCAAATATCAGAGCCACAAAATTACCAAACGAAATTTCTTAACAGTTTTA
AAATAGAAATCTATTTTTTGACTCAGCAGTACATACATATGGTTCTAAATGAGACAGGTGGAAGAGGACA
CAGTGAAAAGGCTGTTGGATAACTTCCACTAGGTCAAGAAATTAAATTGAAAAAATAGCAGCTACTATTG
GAACTATGTGATATACAAGAAAAACAAACTATGGAACTAGTAATATGACAAGAAAAAGACACATGGAAGG
CTCTCTGGGTTTCATAGGAAAAGCATAAAACTAATCCAAGGCAATCAGGAAAACTCCCTGGAGGAGATGA
CACTTAAGGAGAAGTGATGGCCGGGCGTGGTTGCTCAAGCCTGTAATCCCAGCACTTTGGGAGGCCGAGG
TGGGTGGATCACGAGGTCAGGAGATCGAGACCATCCTGGCTAACATGGTGAAACCCCGTCTCTAGTGAAA
GTACAAAAAAATTAGCCGGCTGTGGTTGCAGGCGCCTGTAGTCCCAGCTACTCGGGAGGCTGAGGCAGGA
GAATGGCGTGAACCCGGGAAGCGGAGCTGGCAGTGAGCCGAGATCGCGCCACTGCACACCAGCCTGGGTG
ACAGAGCGAGACTCCATTTCAAAAAAAAAAAAAAAAAAAAAAAGAGAAGTGATATCCAAAATATTTATTT
CAACAATATTTCAGGAGCTCATGCCTGTAATCCCAGCAATTTGGGAGGTCGAGGCGGGAGGCTCACTTGC
ACTCAGGAGTTTGAGACCAGCCTGGGCAACGTAGTGAGACCTCATCTCTAAAAGAAAAAAATAAAAAATA
TTTCAGAGCCGGATGCAGTGGCTCACGCCTGTAATCCCAGCACTTTGGGAGGCAGAGGTGGGTGGATCAC
CTGAGGTCAGGAGTTCGAGACCAGCCTGGCCAACATAGTGAAACCCCACCTCTACTAAAAATACAAAAAA
TTAATTGGGTGTGGTGGTGGGCACCTGTAATCCCAGCTACTAGGAAGGCTGAGGCAGGAGAATCGCTTGA
ACCCAGGAGGTGGAGGTTGCAGTGAGCTGAGATTGCGCCACTGCACTCCAGCCTGGGCAACAAAAGGGAA
ACTCCGTCTCAAACAAACAAACAAACAAAATATATATATATATATTTATTTCAGGAGCTGAGTGTGGTGG
CTTGTGTGTGTGGTCCCAGCTACTCAGGAGGCTGAAGCAGGAAGATCACATGAGGCCAGGAGTTCAAGAC
CAGTCTGGGCAATATAGCAAGACCCTGTCTTTAAAAAAAAACCAAACACCCCCTCAATATTTCAGAACAG
AAGAGTGCTGCCTGTGTTCCTCATAGGGCCACCCCTCCCCCCATGTTAACCCTTTAGTTACAAGATGGAG
GGAAGGTAGGGGCTGTGGACCCCACGTCTATGCTGAGACCCGAATGATAAATAGAGGCTGGTTGGCTGCA
GAAGGTGGGAAGAGGTACAAGCTGAAGTCACAACTCCTGGAAAGAGCCCGAGGCAAGAGAGCAGATGAAC
AAGTGAGACTATAGGGGGTTTAGGGAATAATCAATGAAGAGACTGAGGTCCATTGAGGTGGTTGAAAGAG
AAGAGCTTCTTTCTCCCCCAGCCCATCTTACAGATGGACAAACTAAGGCCCAGAGATAGCCACGGAGCTT
CCTAAAATCACTCAGCCAGGACTAGGACCCAGGCCATCTGACTCCCAGCCCAGAGGGGAGTCAGAAGTGG
GTGTGGAGCCTGCTCTGGGGAGGGGAGGGACAGCCACACTGACCATATCTGTCTGTCTGTCCTGCAGGCC
CGGGGAGAAGGAGAGATGTCTAAATCCCTATGGTTCAAAGGCGGGTAAGTAATGAAACTGTGAGCGGTGG
GAGGAAGATTTGAACCCCAAGTCCAAGGCTGGTTTGCGCCTGAGCCTCAGCCCAGAGAGGAGGAGGAGGG
GTTCCATTTAGACAGGGGACAGATTGAGCTTGGGCAAAATACGTCACTTTCATAAGGCACACAGCCATAC
GCAGACACAAAAGACAGTAATCTTTGAAGACAGGGGGGGGTGCGGTGGCTCATGCCTGTAATCCTAGCAC
TTTGGGAGGCCGAGGCGGGAGGATCATTTGAGGTCAGGAGTTCGAAACCAGCCTGGCCAACATGGTGAAA
CCCTGTCTCTACTAAAAACACAAAAATTAGCTGGGCATGCTGGCAGGCGCCTGTAATCCCAGCTACTTGG
GATGCTGAGGCAAGATAATTACTTGAACCCGAGAGGCAGAGGTTGTAGTGAGCTGAGATTGCACCACTGC
ACTCCAGCCTGGGAGACAGAGCGGGACACTGTCTCAAAAATAAACAAACAAATAAATAAATAAATAAATG
TAAGATAAAGCAGACACTATGAGGAACTAGACACTATTGGGAAATGTACACTATATATTTGTAATCCTGG
AGCAAAATTTTTGCGTTGATTCTATTTTTAAATCTCTTTGATTATTGCTACAGTTGTGGGCTCTTTAATT
AAAGATAGTTGTTTAGTCTTCAGTGGAGAATGTGGTCATGTCTAACCTCTTATGTTATGGATTTTCAGGT
TAGAGGATGCACAAGATATTTTAAAAATATGCTCAGGTTTTAAAATAGCAGCACGATAGTGACCTTCCTT
GTACGTCCTTGGCAAACAGGCAGGGAGACAGCAGCTGGGACTCCTAAATAAATGAGCAGGTACCCTAGCT
AGAGAGCCTTGGATGCGGGTCCTGAAATGCGGATTCCTGTGCACGTGATTTATGAAGGATGTGCTCTCGG
GAGAAACCTGTCAGACAAGGGGAGGCTGCCGAGAAAAGATGTGGTTTAGGAGTCTCTAAGCCTCAGTTGG
CTCCTGCAGAAAGCTTTGTAGGTTGAATAGCCCTGCAGGGCTTGTCTGGAGTCATTGGCTGCAGGCACCT
ATCTCACTGGCCAGTGAAAACCTTTTCGGGGCCAGGCTTGGTGGCTCACGCCTGTAATCCCAACACTTTG
GGAGGCTGAAGCAGGTGGATCGCCTGACGTCAGGAGTTCAAGACCAGCCTGGCCAACATGGCAAAATCCC
GTCTCTACTAAAATATACAAAAAATTAGCCAGGTGTGGTGGTGCATGCTTGTAATCCCAGCTATTTGGGA
GGCTGAGGCAGGAGAATCACTTGAGGCTAGGAGTTCAAGACCAGCCTGGGCAACATAGCGAGACCCCCAT
CTCTACAAAAGAATTTTTTTTTAATTAGCTATGTGTGGGCCGGGTGCAGTGGCTCATGCCTGTAATCCCA
GCACTTTGGGAGGCCTAGGCGGTTGATCACCTGAGGTCAGGAGTCTGAGACCAGCCTGGTCAACATGGTG
AAACCCCATATCTACGAAAAATACAGAAAATTAGCCAGGTGTCGTGGTGCGCACCTGTAATCCCAGCTAC
TTGGGAGGCTGAGATAGGAGAATTGCTTGAACCCAGGAGGCAGAGGTTGCAGTGAGCCAAGATCATACCA
TTGCATTCCAGCCTGGGCGACAGAGCGAGACTCCATCTCAAAAAAAAAAAAAAAAAAAAAATTTGCTGGG
TGTGGCGGTGTACACCTATGGTCCCAGCTACTCAGGAGGCTAAGGTGGGAGGATTGCTGTAGCCCAGGAG
GTGGAGGCTACAGTGAGCCGTGATTGTGCCACTGCACTCCAGCCTGGGTGACAGAGTGAGATCTTGTTTC
GAAAGATGGCTGGGCATGGTGGCTCACGCCTGTAATCTCAGCACTTTGGGAGGCCAAGGTGGGCGGATCG
CCTGAGGTCAGGAGTTCGAGACCAGCCTGGCCAACATGGTGAAAGCTTGTTTCTACTAAAAATACAAAAA
TTAGCCAGGCGTTGTGGTGGGCACCTGTAATCCCAGCTACTTGGGGAGCTGAGGCAGGAGAATTGCTTAA
ACCTGGGAGGCAGAGGTTGCAATGAGCCAAGAAAGGTTTCATCTATATTTTATTTTATTTTTGATACAGA
GTCTCGCTCTGTCACCCAGGCTGGATTGCAGTGGCGTGATCTCGACTCACTGCAACCTCTGCCTCCTGGT
TCAAGCAATTATCCTGCTTCAACCTCCTGAGTAGCTGGGACTACAGGCATGCACTACCATGCCTGGCTAA
TTTTTGTATTTTTAGTAGAGACGGGGTTTCACCATGTTGGTCAGGCTGGTCTCGAACTCCTAACCTCAGG
TGATCCGCCCGCCTCGGCCTCCCAAAGTGTTGGGATTATTACAGGCGTAAGCCACCACTCCTGGCCTCAT
TTATATTTTAGTATATTATTTAGATTTGCCTTAATTCACGTTTTCATTCAAGGTGGGCAATTACATCATC
TCCCCATTTTTCAGATGAGGAGGGTGAGGCCGGGTGTCTCCATTTCTTAGGACTGCTGTCACAAAGTACC
ACAGACGGGTAGCTTAAAACCACAGCAATGTATTCTCTCACAGTTCTGGAGGCCAGAAGTCTGAAATCAA
GGCATTGGTAGAGCCATGCTCCCTCTGAAATCTGTAGAAGAGAATTCCTCCCCTGCCTCTTCTAGCTTCT
AGTTTGCTTGAATTCCTTGGCCCCCGAGTATTGGTTTGTAGATACGTCGCTTTCTGCCTCTGCTGTCACA
GAGCCACCTTCTCCCCATGTATCTCTTCTTCTAAGGACACCAGTCATATTAGATTAAGTGTCTACCCTAC
TCCAGTATGACCTTATCTTAACTAATTACATCTGTGACAACCCCGTTTCCAAGTAAGGTCACCTTCTGAG
GTAGTGGGGGTTAGGGCATCAGCGTATCTTTTTTTTGAGGGACCCAATTCAACCCCTTAACACCTGGGGG
AGGTTGAATGTTCTCCCCAGGGCTCTATGTGAGGAATAGGGACATCTGGGCCCTCAGTGTCCCTCTGTGT
CTTTCAGCCCAGGGGGCGGTCTCATCATCATCGACAGCATGCCAGACATCCGCAAGAGGAAACCTATCCC
ACTCGTGAGCGACTTGGTAAGCACCCTCGCCCTTTACTCCCTGAGCAGAGCAGGCCTGCTATAAGCAGTG
CACAACCTGTGGAACTGCCCATGGTCAACCTGGGGTTGGGGGCCAGCTAGGCATTCTCTGAGACTAATGT
AGTCTGATTTCTACCCCCATGTGCTGCGTGGACACTTCAGGCCATGGTGAGTGATGGCGACATGGGCCTC
TTGGGATCTTCTCCCCAAACTCCCCCACTGGGATCTTCTAACAGGCCCCCAATTCATGGATTATGGGGGA
ACCTTGCCATGTCCCACTGTGTTGGATGCTGTGAGTCAGGGGTGCGGGGCTGCTCACTTTCCAGGGTCCC
TTCTAACTCAAAGCCTCTGTCACCTCCTAATTCTTTGTTGGGTCTGGCCCGGATGCTGTCGGTGACCTGT
TTCCAACTTGCGGTTGTGGCTCCAAAATGTGGCTGCTGGCCCCATCCGTGGCTGTGAACTCTCCTGGTGG
TGATCTCATGGATGTCAACCCCCCCGACTCCCTCTTCTGTGACCTCCCCAGTCCCTGGTCCAGTCCAGGA
AAGCGGGCATCACCTCGGCCTTGGCCTCCAGCACGTTGAACAACGAGGAGCTGGTGCGTGGGGCACCTCT
TTTTCCCCGGGTCAGGGATGGACTGGGCTAGGAGCTTCTGGAATTGTCCAAGCCTCTGTGGGTCCCCGGG
AAGAGGAAGGGTGTGTGTGTGACACAGACTGGCAGAAGGCAGCGTGGATTCCGACAGCGTGGAGGCAGGG
GAGGGCCTGGGACTTTACCGCGCTCAGTGATGGGGCGGAATGGGGCAGGACAGGGACAGCGCCAGGTTAG
GCGCCCCCAAACCGCACCCCATCCCTCCCTGCAGAAAAACCACGTTTACAAGAAGACCCTGCAAGCCTTA
ATCTACCCCATCTCGTGCACGACGCCACACAACTTCGAAGTGTGGACGGCCACCACGCCCACCTACTGCT
ACGAGTGCGAGGGGCTGCTGTGGGGCATCGCGAGGCAGGGCATGCGCTGCACCGAGTGCGGTGTCAAGTG
CCACGAGAAGTGCCAGGACCTGCTCAACGCCGACTGCCTGCAGCGTGAGCGCAGGGCCTGAGCGCAGGGC
CACGGGTTGGCGTGGGGAGCCCCGGGGCTTGCATGGCGGGGAAAGGCTGAAGGGCAAGGCGATGGGTGGG
GCACCATGGGGCGGGGCAAGGGGCGGGGCGTCGTGAGGCAGGGGAGGGACTCGCTGAAAGGGAGGGGTGG
GGTTAAAGGCGCAGGACTCCCTGGTGGAAGGGCTAGGGGGTCGAGGGGTGGGGAAAGAAGCTGTGAGGGT
GTCAGAGAGGGGTGGGGCAGGGCTCAGGGACTAGCTAGGTTGTGCAAAGGACAGGCTTAGTGAGAAGAGG
CAGCTCCCCTGGATAAAATGGGTTAGCTAGGGGTAGCTCTGAGAGGAAGAGGGGCTCAGAGAGGGAAGGG
GTTCAGACTGCGAGAGGACTGTGGAGGGGGGGATTGGAAGGGGGAGGGGACTCAGAGGAGGTGGGACCAA
GAGAAAGGGAGGGGTCTGAGAGATGGAAGGGGCTCAGGAATGTGGAGGGGACTCGCAGGGGTGGAGATCA
GAGACGAGGAGGGGATTCAGAGGAGTGGGGCTCAGAGAGGCAGCGGGACTCAGAGGGGTGGGACTCAGAG
AGGGGGCGGAGATTCAGAGGGGTGGGACGCAGAGGGGGCGGGGACTCAGAGGGGTGGGGCTTAGGGAAGA
GGCCTTAGAGGGGTGGGGCTCCAAAAACCAGTGGGGACTCCAAGGGGTGCGGGGCAGAGAAGGGCAGGAC
GCAGGGACATGGCGACTCAGAGGAGCATGGCTCCGAGAGGAGGTGGAAGGGTGAAGGGTCTGGGTGGGGG
GTACAGAAAGCAGGTAGGAAACCGGCTTCAGGGGGTGACACCTGAGTCCCCCCGGTTGATGAGTAGGGGC
GGGGCTCTCGCCTTGGTGAGGGGCCACTATGCAGGGCGCGCTGGGGTCAGCGCCGGCCTCGGGCGTCCTC
AGGGGCTGCGGAGAAGAGCTCCAAGCACGGGGCGGAGGACCGGACACAGAACATCATCATGGTGCTCAAG
GACCGCATGAAGATCCGGGAGCGCAACAAGCCCGAGATCTTCGAGCTCATCCAGGAGATCTTCGCGGTGA
CCAAGACGGCGCACACGCAGCAGATGAAGGCGGTCAAGCAGAGCGTGCTGGACGGCACGTCCAAGTGGTC
CGCCAAGATCAGCATCACCGGTGAGGGGGCGTGGGCCGCCATAGGGCCCCTCCTTCTCCACCCTCTGAGA
CCTGTCTCATGCCCTGTCCCATGGCCTGGTCCCTCTCCTTTGCAGCGCGCACTCACTCGGTCGGGTGCAT
TCCATCTCATCTCCGTGTGTGCACGCATATGGGGGGACATGCGTGCCTCCAAACGCCTGTTCATGGCACT
GTATCTGGGTGCGTCTTGGATGGTGCCCTCCAGAATGAGACTCCAAGAAGAGGAATTGAGGGTGCATGGT
TTATCTGGAAAGTGACCCCAGAAAGTCCTGGTGAGGGGCAAGGACTTGAGACAGGGATGGAAGGAGTCCT
GAAGGAGATGCCGTTGAGCAGACAATGCTGTGGGCAAGGGGGTCAGCCCATCCTGGGGCCACTGGGGACA
ATGTCGAGCATGCAGCTCAGTGTGGCCCCACTTAGGGGCGAGGATACTGGCTTTTCTCCACACCCAGCCT
CATGTGGGTTTCCCCATAAACAGACCTGAGGATGAGGATTTCAGGTTATGTGGGTTATCCAGGAAGTGAC
CCCAGGACGGAGCTGGGGTGGTAAGTCAGGGAGGGAAGGAACCCTGCAGGGGGCACCAACGAGCAGGAGA
TCTCGGGGGGCAATGGGTTCTTGTAAGTGTCTGACCTCAGGGAGTTCACACCTGAGAGAGGTCCTACCCA
TGCGCAAGGCAGCTGGGGCACCTTCCCATCCTTCATGGTTTGGGGACTCCTTCCCAGCACTTTGGGAGGC
TGAGGCTGAGGCAGGCGGATCACCTGAGGTCAGGAATTCGAGACCGACCTGGCCAACATGGTGAAACCCC
ATCTCTACTAAAAATGCAAAACGATTGGCCGGATGGGGTGGCACGTGCGGGTAATCCCAGCTACTTGGGA
GGCTGAGGCAGGAGAATTGCTTGAACCCGGGAGGCGGAGGTTGCACTCCAGCCTGGGTGACGAGCAAAAC
TCTGTCTCAAAAACAAACAAACAAACAAACAAACACACAAAAAACCTCTCTGGCAAGCCCAGCGTGCCCT
GCCATTGCTCTCCAGCCATGCTGCAGGTGTCCCAATTATCAGCCTGCAGCGTGTGGCAGTGACTGTCCCC
AGCATGCCTCGCTTCTTGAGTGCACACCTGTGTATGCCCTCTGTGTCTTCCAGTGGTCTGCGCCCAGGGC
TTGCAGGCAAAGGACAAGACAGGATCCAGTGACCCCTATGTCACCGTCCAGGTCGGGAAGACCAAGAAAC
GGACAAAAACCATCTATGGGAACCTCAACCCGGTGTGGGAGGAGAATTTCCACTTGTAAGTGCCTGGCTG
GGTCCCTGGCCCCATGTGGGGCATCCTGCTGGGGCAGCCAAAAGAGGGCTCCAAGGACAGGACTCACACT
GGCTGCCTGACCCCTGCCTCTGCCTCCTCCTCCCGCCACAGTGAATGTCACAATTCCTCCGACCGCATCA
AGGTGCGCGTCTGGGACGAGGATGACGACATCAAATCCCGCGTGAAACAGAGGTTCAAGAGGGAATCTGA
CGATTTCCTGGGGCAGACGATCATTGAGGTGCGGACGCTCAGCGGCGAGATGGACGTGTGGTACAACCTG
GGTGAGAGAAGCTGGGTTCTGAAGCGGGTGGGGACGGGTCCCAGGGATCCAGCCAGAGCATGGAGCCCAC
GAAGAGCAGAGAGATGTGTTCTCAGGAGGAGTTTGGTCGAGGAGCATAATCTGATGGGGGAGGCAGGGTC
TAGGAGCCCAGTTTGATGGGAGCAGGAAGCACCCAGGCCAGGAGGGTGAGCAGAGCCCAGGAACCTCTTA
GTTCAGCTTAGGAGGGAGGGTGGGGACAGAATCTCCATGTTCTAGTCTAATGGGGGAGGCAAGAAGCAAG
AACTGGGAATCCTAGTCTGAGGGGAGAGACAGAGACCTAGGAACTAAATCTAAGAAAGAAGATTGAGCTT
TAAGAATCCAGGCTGGGGCCAGGTATGGTGGCTCACGCCTGTAATCCCAGCACTTTGGGAAGCTGAGGCA
GGCAGATCACATGAGGTCAGGAGTTCGAGACCAGCCTGGCTAACATGGTGAAACCCCGTCTCCACTAAAA
ATACAAAAATTAGCCAGGCGTGGTGGTGGGTGCCTGTAGTCCCAGCTACTTGGGAGGCTGAGGCAGGAGA
ATCGCTTGAACCCAGGAAGCGGAGGTTGCAGTGAGCTGAGACTGCGCCACTGCACTCCAGCCTAGGCAAC
GAGAGCAAAACTCCCACTCAAAAAAAAAAAAAAAAGAATCCAGGCTGGGGGCGCCGGGCAGTGGCTCACA
TCTGTAATTAATCCTATCACTATGGGAGGCTGAGGCAGGCCAATCACTTGAGTCCAGGAGTTCAAGACCA
GCCTGGGCAACATAGTGAAACCCCATCTCTATAAAAGATACAAAAATTAGCTAGGCAGGGTGACACTCAC
CTGTGGTCCCACCTACTCTGGAGGCTAAGGTGGGAGGATCCCCTGAGCCTGAGAGGTAAAGGCTGCAGTG
AGCTGTGATCACACTGCTGCACTCCAGCCCAGGGAACAGAGTGAGACCCTTTCTCAAAAAACAAATATGG
CCGGGTGCAGTGTCTCACGCCTATAATCCCAACAGTTTGGGACGGCTGAGGCAGGCAGATCACTTGAGGT
CAGGAGTTTGAGACCAGATTGTCCAACATGGCGAGATCAAGCCCCTGCACTCCAGCCTGGGCAACAAAGG
AAGACTCCGTCTCAAAAAAAAAAAAAAAAAACAAAAGAAAAAGAAAAAAAAAATGGAATCCAGGCTGGGC
ATGGTGGCTCCCACCTGTTATCCCAACATATTGGTTGGGTGGCTGAGCTGGGAGGGTCACTTGAGCCCAG
GAGTTCGAGACCAGCCTGGGCAACATAACAAGGCCTTTGTCTCCATTAAAAAAAAAAAAATAGCTGGGTG
TGGTGATGCGTGCCTGTAGTCCCAGCCACTTGGGAGGCTGAGGTGGAAGCATCATTTGTACCTGGGAGGT
TGAAGCTGCAGTGAGCTATGATCATGCCACTGCACTCCAGCCTGGGCGATAGAGCAAGACTCTGTGTCCC
CCCGACCCCCCACCACAAGAAAAGAATCCAGTCTAAAGAGGGAGGTATTGTTTGGGAATCCTAATCTGAG
ATGGCTGCTACAGATCAAAGAACCCTGTGTAAGGAAGTTGAGTAGAATCTGGGGAGTCCAATCTAATGGA
GGAAGCAGGGTGTATGAGCCTAATCTGAGGGGGTAGAAAGGACCCAGCCTGAGAGAGGTGAGCAGAGCCC
GGAAACCTCTTAGATTGGAGGACATCTAGATATCCCATTCTGATGGGGGAGGTGGGAGACAAGACCAGGG
GGTCCTAGTCTGAGGAGAGAGGCACAGACCCAGGAACAGTGTCTAAGATGTGTAGTCTCCGGGGAGCATG
ATCTGATGGGGGAGGCAGGGCCTGGGAGCTCAGACTGGGAAAGAAGTGGGGCCTAGGGCTGCTAGTCTAA
GGAGGGAGGCACATAGCCAGGAACTTAATCTGAGTGAAGAAGCAAGGTCTGGGATCCTTGTTTGAAGGAA
GAGACAGGGCTTAAGGAATTCTAGCCTAAACAGGGAGACTCAAGCCTAGGGAGCCAATCAGAAGAGAGAC
ACCAAAGCCTTAGTTTCCGGAAGTCTGGGAGGACTACAGAATCGTGTATCTATTAAAAATTGCCTTGGGA
GGCCGAGGCGGGCGGATCACCTGAGGTCAGGAGTTCGAGACCAGCCTGGCCAACATGGTGAAACCTCGTC
TCTACTAAAAATAAAAAATTAGGTGGGTGTGATGGCACATGCCTGTAATCCCAGCTACTCGGGAGGCTGA
GGCAAGACAATTGCTTGAACTCAGGAGGTGGAGATTGCAGTGAGCCAAGATTGCGCCACCGCACTCTAAC
CTGGACCACAGATCTCAAAAAAAAATAAAATAAAAAAAAATTTGCCCATGGCTGGCCGGGCGCGGTGACT
CACGCCTGTAATTCCAGCACTTTGGGAGGCTGAGGTAGGTGGATCACCTGAGGTAAGGAGTTCGAGACCA
GCCTGGCCAACATGGTGAAACCCCGTCTCTACTAAAAGTATAAAAATTAGCTGGGCACGGTGGCAGGCAC
CTGTAATTCCAGCTAGTTGGGAGGCTGAGACAGGAGAATCGCTTGAACCCAGGAGGTGGAGGTTGCAGTG
AGCTGAGATCGTGCCACTGCACTCCAGCCTGGGTGACAAGAGCAAGACTCCCTCTTAAAAAAAAAAAAAT
TGCCCACTGCTATAGTGGGAAGTTCTGACACTGTCTTCTTTTTCTGCCCTGCAGACAAGCGAACTGACAA
ATCTGCCGTGTCGGGTGCCATCCGGCTCCACATCAGTGTGGAGATCAAAGGCGAGGAGAAGGTGGCCCCG
TACCATGTCCAGTACACCTGTCTGCATGAGGTGAGGGTCATTGCTCGGCCCCTCCCATGCCACTTCCACT
CACCATTCCTGCCTGCCCAGCTCTTCCTCTTTCTGGCCACACCATCCACACTCTCCTGGCCCTCTGAGAC
TGCCCGCCATGCCATTCCCTTTACCTGGAAAACTCCTCCCTATCCATCAAAGTCCAGATTCAGGGTCACC
TCCTCTGGGAAGCCCACCTTGGCCTCCAGGTTGACTCTCACTACTCATCATCAGGTTCTTCCTTCTATTC
CAGCCCTAACCACTCAGGATTGGGCCGTTTGTGTCTGGGTATGTCTCTTCCAGCTGCCTGGGTTTCCTGG
AAAGAACTCTTATCCCCAGGAACTAGTTTGTTGAATAAATGCTGGTGAATGAATGAATGATTGAACAGAT
GAATGAGTGATGAGTAGATAAAAGGATGGATGGAGAGATGGGTGAGTACATGGATGGATAGATGGATGAG
TTGGTGGGTAGATTCGTGGCTAGATGGATGATGGATGGATGGACAGATGGATGGATATATGATTGAACTA
TTGAAAGTATAGATGTATGGATGGGTGAATTTGGGGGTAATTGTTAGATGATGGATGAGTATAGATGAAT
GATGGATGGATAACTTGATGAGTGGATAGATAGATTGCTGGATAGATGATTGACTGGGTGGATAGATGAA
ATGTTGGATGAGCAGATTAAGTTGTATTGGATGGGATGGATGGAAGTGTGGTTGAGTTATTAGAAGGAAG
ATTGAGTAGATAGGTGAATTTGTTGATAGTCAGATGGGTAGATAGGTAGATGGATGGATGGATGGATGGA
TGTATAGGCAGATGGACAAATGGATGAATGGGTGGGTGGATGAATGGAAGGATGTGTGGTTGAACTATTG
CAAGTATTGATAATTGGGTTCATAATTTCTGAATATTTAGATGGATGGTTGTGAGTGGCTGGTGGACAGA
CGAAAAATGGATGGTTGGATAAATTGATGGGTGGATGGATGGTTGGTTGTATGAAAGAATGAATGATTGG
GTAGGTGGATTAAGTTGCGGATCAATGTATGGGATGGATGAATGGATGGATGGATGGATGTGTGGTTGAA
TTACTGAAAGGTTGGAAGAGTGGATGGGTGAAATTTGGGGTAGTTAGATGGGTGGGTGTGTGGATGGATA
AAAGAGTAGATGAATGAATTAATGAATAAACAGGCAGATGGATGATGTAAGCTGCCCCAGACCCTGGGAC
CTCTGACCCCCGGCGACCCCTTGCACTCTCCATGACACTTTCTCTCCCATGGTGGCAGAACCTGTTCCAC
TTCGTGACCGACGTGCAGAACAATGGGGTCGTGAAGATCCCAGATGCCAAGGGTGACGATGCCTGGAAGG
TTTACTACGATGAGACAGCCCAGGAGATTGTGGACGAGTTTGCCATGCGCTACGGCGTCGAGTCCATCTA
CCAAGCCATGACGTGAGACTGCAGGCTGGGGTGCAGGAAACAAGAGGGAGGTCACCAAGTGGGCACTGGC
AGGTCTGGGGTGGGAGGGATTCTCAGGGGGAAGACCCAGAGAGGAAGTGGGCGTGGTGGTGGTGGGTTCC
GTAATTCCCCTGGAGATCAGGAGAGCGCTGTGATTGACACCTGGAGTGACATGGAAGAGAGGGAAGACTT
AGAAGCAGAGATAGAGGCTGGGCACAGTGGCTCAAGCCTGTAATCCCAGCATTTTAGGAGGCTGAGGTGG
GCGGATCACTCCAGGTCCAGGAGTACAAGACCAGCCTGGCCAACATGGTGAAACCCCATCTCTACTAAAA
ATACAAAAGTTATCCAGGTGTGGTGGCTTGGGCCTGTAATCCCAGCTACTCGGGAAGTTAGGGTGGGAGA
ATTGCTTGAACCTGGGGGGCAGGGGTTGCAGTGAGCCGAGATTGCGCCACTAAACTCCAGCCTGGGTGAC
AGAGTGAGACTCCATCTCAAAAAAAAAAAAAAAAGAAAGAAAGAAAAAAAGAAGCAGAATTAGAGCAACT
CAAATGGATATAAAGATGGAGGGGAGAGGAGATGCGGAGACCCAAAGGACAGAATCAGGCAGGGGATGGT
TTAGGAGGTGACAGAGACCCACTCACAGATGCTATCTTGGAGGCATTCTAGGGGGGATCTTGGTCCATCC
TGGCTGGACCCCTGGCCTAGTGCCTCCTGCTCCCTCTCTCCAGCCACTTTGCCTGCCTCTCCTCCAAGTA
TATGTGCCCAGGGGTGCCTGCCGTCATGAGCACCCTGCTCGCCAACATCAATGCCTACTACGCACACACC
ACCGCCTCCACCAACGTGTCTGCCTCCGACCGCTTCGCCGCCTCCAACTTTGGGGTCAGTCCTGGGGACT
GGGATTGGAGAATTAGGGAGAGCCCAACTTTATGCCGGTCAGATCTGATGTGACTTCTGGGTCCCCAGTC
TAGACCTGATTGCTGTGTGACTTTCCACCAGTCACTTCCCGTCTCTGAGCCTCCATTTATTCCTCTGTAG
AATGCTATAGAATGGAGGTCGGGCGCAGTGGCTCATGCCTGTAATCCCAGCACTTTGGGAGGCCGAGGCT
GGAGAATCACTTGAGGCCAGGAGTTCGAGACCAGCCTGGCCAACACGGTGAAAACCCGTCTCTTCTAAAA
ATCCAAAAGAAAATTAGCTGGGTGTGGTGGCCTGTGCCTGTAGTCCCAGCCACTTGGGAGGCTGAGGCAG
GAGAATTGCTTGAGCCTAGGAGTCGGAGGTTGCAGTGAGCTGAGACTGCGCCACTACACTCCAGCCTGGG
CAACAGAGCAAGACTCTGTCTCAAAACAAAAAAAAAAATGCCATAGAATGGAAATGGACATTATGAGGGT
GGTGGATGAGGTATACTTACTGTACAGTAGGCGCTCAGCCACTATGGCGGCCATGTCCTGCCTCCATCCA
TCCTCCCTCCTCCCCTTCCTCCTCAGAAAGAGCGCTTCGTGAAACTCCTGGACCAGCTGCATAACTCCCT
GCGGATTGACCTCTCCATGTACCGGGTAGGAAGTGTGTGCATGAGAATTTGCTCACCCCACGCACATGTG
TGTGCCTGGAGGGGAAAGCTACCAGCAGGTGTGTGCATGGGAGGACCCATCTTCGCATGTACGATGTGTT
CCTCCAGCATGTGTGCACCTGCATGGGCACTACCTGCCGTAATGTGCTTGGTGCATCCAAGCAGATCCCC
AGTTGCTTCGAGGTGCTCTAATCAACCCCCAGCCTGAAACCGGGGAGCCTTTGTATCCCCCTGAAAAGCA
GTCTCCCATCCTCCCCACGCCTTCTTCCATAACCCCACATCTCTTCCTGCTCCCAACAGAATAACTTCCC
AGCCAGCAGCCCGGAGAGACTCCAGGACCTCAAATCCACTGTGGACCTTCTCACCAGCATCACCTTCTTT
CGGATGAAGGTAGGAAAGGATCCAGTTGCCTTTGCTCTCCAAGGAGGGTTCTCTAGGATCCCCAGCTCCC
AGGACAAGTGACATTCCTCCTGGAGAGTGGCAGCCTTTCTCAGTAACCTTTTCCTTTCATACCACTTCAT
TTCCTTAAACCCAAAATGACTCGCAGTCACTTCCTGGGGACCTCCTGTCACAGCTGTGGCCGCCTATGCC
TCTCTCCCTCCCTTCAGGTACAAGAACTCCAGAGCCCGCCCCGAGCCAGCCAGGTGGTAAAGGACTGTGT
GAAAGCCTGCCTTAATTCTACCTACGAGTACATCTTCAATAACTGCCATGAACTGTACAGCCGGGAGTAC
CAGACAGACCCGGTGAGACTCCAGATGGGTCAGAAGGGAACAGTGATGCCAACTAGACACAGGCTTCAGT
CCCAGCCCTGCCCCTTCCCGAGGGATTTAACTCTTCTGAACCTCAGTATCTTCTTCTATAAAATGGGGTC
ATTAACCCACATCTCAGGATTAGGGAGAAGAGTTCATGAGCTGTAAGTTTAGATAAAATATAGTCGGCAC
TCAGTACATTTGGTTGCGTAGTTGAGTTTGGCATAAAAAAAAATTTTTTTTTTTTGAGATAAAGTTTCAC
TCTTGCTTCCCAGGCTGGAGTGCAGTGGTGTGATCTCAGCTCACTGCAACCTCTGCCTCCCAGGTTTAAA
TGATTCTCCTGCCTCAGACTCCCGAGTAGCTGGGATTACAGGTGCCCACCACCACACCTAGTTAATTTTT
TGTATTTTTAGTAAAAACGGGGTTTCACCATGTTGGCCAGGCTGGTCTCGAACTCCTTGACCTCAGGTGA
TCCACCTGCCTCGGCCTCCCAAAGTACTGAGATTATAGGCGTGAGCCACCACTCCCAGCCTGGCATAAAA
ATTTATGTCTGGCATACAGTCACTGTTCAGTAAATGTTGAGTTGAATTGAATTTATTTATTTATTTTGAG
ATGGGGTCTTGCTCTGTTGCCCAGGCTGGAGTGCAGTGATGCAATCATGGCTCACTGCAGCCTCGACCTC
CCAGGCTCAAGTGATCCTCCCACCTCAGCCTCCCAAGTAGCTGGGACTATAGGCACGCACCACCATGACA
GGCTAGTTTTTGTATTTTTGTGGAGATGGCATCTCACTATGTTGCCCAGGCTGATCTCCTGGGCTCAAGC
GATCCTCCTGCCTCAGCCTCCCAAAGTGCTGGGATTACAGGCATGAGCCACCACCTGTAAGGAGCTTATA
GCCTATTATGAGGGAAAGATGTACAAACTGGAAATTTACAAACAGGGATGGTTAGTATTGAGATAGTGGG
ATCTCAGGAGGCTGTGGGAGCTGGAGGCAGCCCCTACCCAGGTTCTGATAGGATATAACACATAAGCTGT
GTTCAGTTCAATTTGATTTCATTCTGTTCCTTTACATTCAGTTGACTAGAGTTGGAATCAGTCTCACTCT
CATTTGAATTGAGTTGAGGTGAACTGGATTAAATTAGGCTATAAGGAGCTTATAGCCTATTATGAGGGAA
AGATGTACAAACTGGAAATTTACAAACAGGGATGGTTAGTATTGAGATAGTGGGATCTCAGGAGGCTGTG
GGAGCTGGAGGCAGCCCCTACCCAGGTTCTGATAGGATATAACACATAAGCTGTGTTCAGTTCAATTTGA
TTTCATTCTGTTCCTTTACATTCAGTTGACTAGAGTTGGAATCAGTCTCACTCTCATTTGAATTGAGTTG
AGGTGAACTGGATTAAATTAGATTGTTTAGTTCAACCATAATCAGTTTAGTTATATTTATTTCTATTTGG
TTGACTTAAGTTAAAATTGTTCTTGACGGCCAGGCACAGTGGCTCACGCCTGTAATCCCAGCACTTTGAG
AGGCCGAGGTGGGTGGATCATGAGGTCAGGAGATCGAGACCATCCTGGCTAACACAGTGAAACCCCGTCT
CTACTAAAAATACAAAAAAATTAGCCGGGCGTGGTGGCGGGCACCTGTAGTCCCAGATACTTGGGAGGCT
GAGGCAGGAGAATGGTGTGAACCTGGGAGGCAGAGCTTGCAGTGAGCTGAGATCGCATCACTGCACTCCG
GCCTGGGTGACAGAGCGAGACTCCGTCTCAAAAAAAAAAAAAAAATTGTTCTTGACAAATTAGTGAGCCA
GGCTGGGCTCAGTGGCTCACACCTGTAATGTCAGCACTTTAGGAGGCTGAGGCAGGAGGATCCCTTGAGG
CCAGGAGCTCGAGACCAGTTTGGGCAACATAGCGAGACCCTATCTTTGCAAAATATACAAAAATTAGCCA
GGTGTGGTGGCGCACCTGTAGTCCTGGCTACTTTGGAGGCTGAGGTGGAAGGATCACTTGAGCCCAGGAG
TTCGAGTCTATGGTGAGCCATGATTGCACCACTGCGCTCCAGCCTGGGTGACAGAATGAGACTCTGTCTC
TTGAAAGAAAAAAAAAAAAAATAGAGAGAAGAACAAGTTGGTTGATTGCATTGAGTTTTATTGAGTACAA
ATTCATTCATTTCCATTTAGTTCATTTACATTCAAGTCAAGTTGTTCTCAACTGGGTTAAATTGTCTTGA
GTTAGGATGAACTGGGTTGTTGGATTAGATGCAATTGATTTCAGTTGAATTGGGTTGAATTGGGCTAATT
TCAGCTGATTATATTCATTTCCATTGATTAACAAAGGTGGGTTGAATTGCCCTGCGTATGTTCAGTTGGG
TTTAGATAGGTAGAATCGGATTGGGTTGACTTGGGTAGAGTTGAGTTCGAGTCGATTTATTTCCATTATT
TTGATTTGGGTTGATGTTGATCTTGGACAAGTTGATTAGGTTCAATCCAACGGAATTCATTTGAGTTTAA
TTCAATCCAGTTAGGTTGAATTGCACTCCATTCAGTGTGATTGGCCCAAGTTCAGTTCCATTGATTTAAC
TCAGAGGAAGAACTTACTTCCGTTAAGCTAAGTGGGCTTAATTTGCATTCATTTCATTTCCATTTGGTTT
AGTGGAGCTGGGTTTGTTCAACTAAATTTGGTTTCATTTATTTCCATTCACTTTTAGAGTGGAGTCTAAA
AATCTTAAGCTTTATCAAGCTGCATTGACATGAGCTCTGTTTGGGCCAGTTTATGAGTTCAATTCCTCTC
TATATAGATTTCTTACCTGAACCATACACATGATGATGGGATGCATTCCTCTAAAACCTCTTGTTCTTCA
GTAATAAACTGACCGAGGCACCAACCTCTGGACCCCCTATAACCGAGGTCTCCACTGTCCATTTCAGGCC
AAGAAGGGGGAAGTTCTCCCAGAGGAACAGGGGCCCAGCATCAAGAACCTCGACTTCTGGTCCAAGCTGA
TTACCCTCATAGTGTCCATCATTGAGGAAGACAAGAATTCCTACACTCCCTGCCTCAACCAGTGAGTTGT
GTATCTGTGTAGTTAATTATCTGGGTGTCATCGTGTGTATGTATACAACTTTGTGTCTTGTGTGTCAAGA
TGATTTTGTCTAATTACACAAATGTGCACCTGGGGGTAATTGTGGGTCTGATTATACCTGTGCAAAATTC
AAATATGGGTACAATTTGCAAACATAAACATAAAATTGTGTGTATGTTCACAATCACTGGTATAATCAGA
CATGTATTTTGTATGTATTCATTTGCATGCATAACTGTGTGTACATATGTGTATAATTTTTTATGTGTAT
GTAGGTTAATTAATAATAGTGTTGCTCAAGTATTACTATGTGCTACCCTAATACATATCATACATTTAAA
TTGCATTATATCTTATATGTATGGATGTAAGTAATTTTGTATATTGTTGTGGTCAGTGGGTTTTGGGTTT
AATTTGTATATGTGTAAAATCATTGAATGCAATTCAACGTACAATATTGTACAATATACAATATTGTGTA
TTGTGTATGAATATGGGCACAGTTTTCATTTGTGGATTTATATGTCTGTAGAATTTTCATCTGTGTTAGC
TGAATGAGCTACTGTGTTCTATTTCTCTGCCAGATGAGTGCTAGAGAGGACTCTAGCTTTGGGGCGTTAA
CATATCCTGGTTAGGGAATTAAGATCAGGGATTTGGATGGATTGAAGGGTGGGTGGATGAATGGATGGAT
TAATAAACAGATGGGGTCAGGCACAGTGGCTCACGCCTGTAATCCCAGCACTTTGGGAGGCCAAGGTGGG
TGGATCATTTGAGGTCAGGAGTTTGAGACCAGCCTGGCCAACATGGTGAAACCCTATCTTTACTAAAAAT
ACAAAAATTGGCCACGCGCGGTGGCTCACCCCTGTAATCCCAGCACTTTGGGAGGCCGAGGCAGGCGGAT
CATGAGGTCAGATGGAGACCATCCTGGCTAACATGGTGAAACCTTGTCTCTACTAAAAATACAAAAATTA
GCCGGGAGTGGTGGTGGGCGCCTGTAGTCCCAGCTACTCGGGAGGCTGAGGCAGGAGAATGGCGTGAACC
CAGGAGGTGGAGCTTGCAGTGAGCCGAGATTGGCCACTGCACTCCAGCCTGGGCAACAGAGTGAGACTCC
GTCTCAAAAAAAACAGAAAAAACAAAACAAAAATTAACCGGGCGTGGTGGTACACGTCTGTAATCCCAGC
TAGTGGGAGACTGAGGCAGGAAAATTGCTTGAACCCAGGAGGCAGAGGTTGCAGTGAGTCGAGATTGCGC
CACTGCACTCCAGCCTGGGGTGACAGAGCGAGACTCCGTCTCTAAATAAATAAGTAAATAACAGATGGAC
AGATAGATTGGTAGAAGGATGACTGTGTGGATAAAGGAACGGATGGGCCAGGTGTGGCAGCTCACGCTTG
TAATCCCAGCACTTTGGGAGGCCGAGGCGGGAGGATCACCTGAGGTCAGGAGTTTGAGACCAGCCTGGCC
AACATGGCAAAACCCCATCTCTACTAAAAATATAAAAAATTAGCCGGGCATAGTGGCTCGCGCCTGTAAT
CCCAGCTACTTGGGAGGCTGAGGCACGAGAATCATTTGAGCCCGGGTGGTGGAGGTTGCAGTGAGCTGAA
ATCGCACCACTGCACTCCAGCCTGGGTGACAGAGTGAGACTCCGTCTCTAAATAAAAAAAAAGGAATGGA
TGGATGGGTGGACAGATGGATGGGTAAATGGATGGGTGGATAGCTGCATAAGTGAGTAAATAAATGGATA
GAGGGATGGATGGATAGGTGGATGGATGAATAGATAGATGGGTCATTTAATGGGTGGGTGGATAGTTGTA
TGAGTGGGCGAATGAATGGATGGAAGGACAGATAAATAGGTGGATGATGGAAGGATGGACGGATGGATGG
ATGAACAGATAGACGGGTAGGTGGCTGGATGGACAGTTGGATGAATGGGTGGAGGGATAGATGGATGAAC
AGGTAGATGAGTGGTTGAGTGGATGGATGAATAGTTGGATGAATGAGCAAATGAATGGATGGAGGGATGG
ATGGATAGATGAATAGATAGATGGATAGGTGAACGGATGGGTGGATAGTTGCATAATTAGGTGAATGAAT
GGAAAGAGGGATGGATGGATGGATGGGTGGATAGATGAATAGATAGATGGGTAGGTGGATGGATGGATAG
TTGGATGAATGGGTAGAGGGATAGATGGATGAACAGATAGATGGGTGGTTGAGTGGATGGATGAATAGTT
GGATGAATGAGTGAATGAGTGGATGGAGGGATGGATGGATAAATAGATAGATGGATAGGTGAATGAATGG
GTGGATAGTTGCATAAGTAGGTGAATGAATGGATAGAGGGATGGATGGATGGGTGGATGGATGAATAGAT
AGATGGGTAGGTGGAGGGATAGATAGTTGCATGAGTGGGTGAATGAATTGATGAAGGGATGGTTGGATGG
GTGGATAATGGGTGGATGGATGAATAGATAGATGGGTACATGGATGGGTGGACAGTTGCATGAGTGGGTG
AATGAATGGATGGAAGGATGGATGAATGGATGGAGGAATAGATAAATGAGTTGTTGAATGTATGGGTGGA
TAGTTGCATGAGTGGATGAATGAATGAATGGAGGGACAAATGGATGAGTGGGTGGAGGCTCAAAGGGAAA
CAAAGCCTTAGTGCAGAGTCAGTCAGCAGTGGCTATGAGGGAACCTGAACCCAAATCCCTCTACCCTACC
CTACACTCCTCTGTGGGGCAGGGAAGGACAAAGCAGCCCATCCCATCTGCCTTCTGCCTGCCAAAGTTTT
ATACTTCCATGACTTTGCCAGGTTTCCCCAGGAGCTGAATGTGGGTAAAATCAGCGCTGAAGTGATGTGG
AATCTGTTTGCCCAAGACATGAAGTACGCCATGGAGGGTGAGTTTCCCACCCCAGTGTTCCCCAGCCTGG
CCACACCAGGTGGCTGTACCTCTGTGTCCCCTCCAAGGCTGTGAGCACCCTATAATACCCTCATGCCTGA
GTTGGAAATCAGTGATGGCCAGAGTAGGCAGCCTCTCTGCCGAGACAGAGGGTGGCAGACCCTTCCAGCT
GTGCCATCCTCTTTGTCCCTGGCCAGAGCACGACAAGCATCGTCTATGCAAGAGTGCCGACTACATGAAC
CTCCACTTCAAGGTGAAATGGCTCTACAATGAGTATGTGACGGAACTTCCCGCCTTCAAGGACCGCGTGC
CTGAGTACCCTGCGTAAGTCCCCTGCCCCAGGCCCAACCCAAGCCAGAACTGTAGCACTGACAGCTAGAA
GGAAAGCCAGAGAGGAAGACTGACCTACTTAGGGTTACCTGGGCATCAGGGGCATGGGTGGGGCTTGAAT
CCAGTGTTCTCCACTCTCAGCCATTGAGCAGTTGACTCCTACAGCCCAGAAAATTCAGTCAACAGCAACA
GGAACCATGTGACTTTCTCATTTTATCCTTTTGACAATTCTATGAGTTAAGGTGGGATCATTTTAATTCC
TATTTTGCGGAGGAGAAATTGGAGAGTTAAAGATGCCAAATGGCCAGGCATGGTGGCACATGCCTGTAGC
CCCGGCAATCTGGGAGACTGAGGCAGGACTTGCTTGAGCCCAGGAGTTCGAGACCAGCCTGGAAAAGATA
GTCAGATCCCATCTCTAAAATTAATGAATGGGTCAGGCACAGTGGCTTGTGCCTGTAATCCCAGGACTTT
GGGAGGCCGAAGTGGGCAGATCACCTGAGGTCAGGAGTTCGAGACCAGCATAGCCAATAGGGCGAAACCC
CAGCTCTACAAAAATACAAAAATTGGCCGGGCGCAGTGGCTCATGCCTGTAATCTCAGCACTTTGGGAGG
CCAAGGTGGGTGGATCACCAGAGGTCAGGAGTTTGAGACCGGCCTGGCCAACATGGTGAGACCCTGTCTC
TACTAAAAATACAAAAATTAGCCAGGTGTGGTGGCAGATGCCTGTAATCCCAGCTACTTGGGAGGCTGAG
GCATGAGAATTGCTTGAACCCAGGAGGCGGAGGTTGCAGTGAGCCGAGATTGCACCACTGGATTCCAGCT
GGGGCAACAAAGTGAGACTCCGTCTCAAAAACAAAGAAAAAAATTATCCGGGTGTGATGGCACACACCTG
TAGTCCCAGCTACTCGGGAGGCCGAAGCAGGAGGATCGCTTGAACTCAGGAGGCAGAGGTTGCAGTAAGC
GGAAATCGTGTCACTGCACTCCAGCCTGGGCGACAGAGTGAGACTCCATTTCAAAAAATAAATAAATATA
AAAATAAAATGAATGAGTGAATAAATGCCACATTCCTTGCCCACGGTGACTCCCAGTACATGACAGACCC
GAGATTTGAACTCGGGACATCTGATTCCTGGGCTGTGTGTTGGAGGATAGTACGGTGCCGCCGGTTCCTG
AGAATTGTGGTGTCGTTCTCTGCTCCCAGCAGCACAGGAGGATGGCTAGTCTTCTGTTGTACAAGAAACG
AAAGCTCTGGCTGGATGCGGTGGCTCATTCCTGTAATCCCAACATTTTGGGAGGCCAAGGTGGGAGGATC
ACTTGAGGCCAGGAGTTCAAGGCCTGCCTGGGCAACACAGGGAAATCCTGTCTCTACAAAGAAGACAAAA
AAAAAAATTAGCCGGGCATGGTGGCATGTGCCTGTAGTGCCAGCTATTCGGGAAGCTGAGGTGGGAGGAT
TGCTTGAACCCAGCAGGTCAAGGCTGCAGTGAGCTGTGATCGCACCACTGCACTTTAGCCTGGGCAACAG
AACAAGACCCAGGAAGGAAGGAAGGAAGGAAGAAGGAAGGAAGGAAGGAAGGGAGGGAGGGAGGGAAGGA
AGGAAAGGAGGGAGGGAGGGAGGGAGGGAGAAAACAAGAGCTCAGAGAATAAACCCACTTGCCATGGGCA
GTTTATCTGAAGAAGGAAGGAAGGAAGGAAGGAAGGAAGGAGGGAGGGAGGGAGGGAAGGATGGACGGAG
GACTCAGAGAGGACTCAGAGAATAAACCCACTTGCCATGGGCAGCTTATCTGAACTAGGATTCCCACAGG
CCTGGGCCCTGTTGTAAAAGGGGCCACTCAATCTATTTCATGAGCAAAGTAACTGAGGTTCAGTGCAGGA
GGCGAGCAGAGGAGCAGCCACTCAAGGCTGCAGGGAGAGCTCCAGGCAGAGCAGGAGCAGCCATAGCTCC
ACTCGTTAGTGGGTCAGAACCAGGCGGCGCAGAGGTTGAGGAGGACAAGAGGTGGCAGAGCCCCACCCCG
GCTCTTCATTCCAGATGGTTTGAACCCTTCGTCATCCAGTGGCTGGATGAGAATGAGGAGGTGTCCCGGG
ATTTCCTGCACGGTGCCCTGGAGCGAGACAAGAAGGATGGGGTAAGAAGGCCCACCCACAACCAAGTTCC
TAATCTTCCCACTGGGGTCAATTTAGGTGGAAATTTGCCCTTGAGATGAGGCCAGTCTCTGGTAGGTGTC
CGCCATCTTTGTCTTGCTCATGGTTTTTAAAATCTTTTGTTCATTCATCAACTACAAAATGTGAGTTCCA
TCAAGCCGGACACCTCCAGGCACCCCATGGCTTCTGCGTCCACCTCTAGAGTTGACTCTAAGTCTACCTC
GCCCTCTGTCCATCTCACCCCGGGGAAATTGGTCTCTGGCTCTCCCCGTGGAGAGTGGGAGAGTTGGGCT
CTAGGGATTCTCTGGTGCTGATCTCCTCCTTCCTCTTGGCCTCCTTCCCCCTGCAGTTCCAGCAGACCTC
AGAGCATGCCCTATTCTCCTGCTCCGTGGTGGATGTTTTCTCCCAACTCAACCAGAGCTTTGAAATCATC
AAGAAACTCGAGTGTCCCGACCCTCAGATCGTGGGGCACTACATGAGGCGCTTTGCCAAGGTGTGGGAGA
GTGGGTAGGGTTGGGGCTAGGGGACAGGGTCAGGGTTAGGGAATTGAAGTCTGAGTTGGAGATGGGCTAA
GGTTGAGGTTGAGGTTGGAGTTGGTGATGTCATTGAGGTTGGGATTGAAGTTGGAGTTTGGGTTGAGGTT
GGAGTTGAAGTTTGGGTTGAGGTTGAGGTTGAGATGAGGTTGGAGTTGAGGTTTGAATGGAGGTTGGAGT
TGGGGTTAGGGTTTAAATTGAGATTGGAGTTGAGATTGGGGTTGGAGTTGAGGTGTAGGGTAAGGTTGGA
GTTGAGGTGTGGGTTGAGATCAGGGTTGGAATTGAGGTCATTAGTGCGAGTTGGCAATGCTGTTTGGTTG
GAGTTTGAGGCTGTGCTGGGTCTTGATTTGGAATTGAGATTGGAGTGAGTGCTGAGATTGATGCACAATT
TGAGTGAGTCTTGGGGTGAGTTTGAGGGTTGGGATCATTGGGGTTGAGGTTTGATTCATTGGCATTAGGC
TGTGTTGACCTTGGGAATTACAAGTAGGAATTGGCTTTTGATTTGAGATCAGGTCATCTTGTGGTTGAGG
CTGGGTTAATATGATGCTTGGTTGCTCTGAAAAAGAGCTGGGTCTACATAAAATGATCATCTTCATCGAG
CGGTATCTCCTCTAGGTCTTGATGGTCATCTTGAGTGGAGAATTTGCCTAGCCCAAAGGCCCATCTTAGG
TCCTAATAGGGTTTGGGTTCATCACTGATCTCACTAGTGGCCTTGATGGGACGACTGGCGCCTGCCTTGG
TGCCAGCCTCTCCTCTCACCCACACTCTGTGTCTTCCCCAGACCATCAGTAATGTGCTCCTCCAGTATGC
AGACATCATCTCCAAGGACTTTGCCTCCTACTGCTCCAAGGAGAAGGAGAAAGTGGTGACCTGAGGGGGC
CCCTGACCTATCTCCCTCCTCAGCCCCATTCAGCATCCCCAGGACTCCCTTCTCCCCAGGGCCCTGACAC
CTGGGGCTATGTATGTCCACCTGTGTGTGATCTGACTGGATTGTCTGTGTGTTTAGAGGTGCTTGTTTGA
ATGTGTGTGATCAGAGTGTATCTAGTTGTGTGTGTCTGAGTGTATGGGAAGTAAGTGCATGTGTCTGACT
GTGTATTGTCTGTGTTGACTGGTATACTCTGCGTGTGTGTGTGTGTGTGAGTCTGTGCACATCTGAGTGT
GCGTGTCTGATCATGTTTTGTCTGGCTTTGTTAACAGGGTATGGCATGAATGTGTGTGTGAGTCCAACTG
TGCACGTCTGAGTGTGCGTGTGTGATTGTGTTTCGTCTGGTTGTGTTAACTGGGTATAACATGAGTGTGT
GTATCTGTGTGATTGTGTTTCGTCTGGCTGTGTTAACTGGGTATAACATGAATGTGTGTATCTGTGTGTG
TCTGTACATATCTGAGTGTGGGTATCTAGCCATGTTTCATCTGTGTTAACCGGGTATAGTGTGAGTGTAT
GTATGTGTGAGTATGTGTGTTCAACTATACAACACATCTAGGTGTGTGTCTCATATTTTATCTAGCTGTG
TTAACTGGTTATAGTGTGAGTGTGTGTATGCTTGTGTGTGTGTCCAGCTGTGCATGTCTGGGTGTGTGTG
ACCGTGTTTTGTCTGGCTGAGTTAACTGCATATAGTGTGAGTGTGTGTGTGTGTTTGTGCACGTCTGGGT
GGGTGTGTCTCACCAGGTTTGGTATGGCGGTGTTGAATGTGCGTGGTGTGAGTGTGTGTCTATGGCTGTG
TCTGGCTGTGCATGTCTGAGTGTGTGTATCTGTGGGGGGGGTCTCGTGTGTATATCTCGGGGTGAGGGCA
CACGCCTTCAGTGTGTCACTGTCTGGCTGTTGCAAATGAAGCCTTCAGGGTGTGAGCTGCAAAGCTTTGG
GCTCTTGCAGGAAACTCTGCTACTCCCAGAGTTCCCAGCCCCTCTCATGGACCCCCCACCAGGCCTGGGG
TCGTGGGATTACTTGAGTCCTCCTTGCCCCCACCAGTCCCCAGAGATGCCCGCCCCAACAACACGCCCAG
GAACCTGAGCACAAAAGCTCCCTTCCATTTGCAGGTGACCCCATGGGACCCCAGCTTCCTCCAGACCCAG
ACCCTGAAACCATCCCAAGGTCCCTGGCGGTGGGTAGGGGAGGCTCTGGGGAGAGGTCCTGAGGCTTGAC
TCCCCTCTCTGTTCCCACCCACAGCCCTGCATTCTCATGAATAACACTCAACAGCTACGAGTTCAGCTGG
AGAAGATGTTCGAAGCCATGGGAGGAAAGGAGGTGAGGCAGGGCCGAGGCAGGGCTGGAGAAGGGATGGG
GCACCCCCACCACTGTGTCCCTGCAGGCCCCTGAGCTCATATGCCACCAGGCCTGGGGATGAATTCCAGC
TCAACCATGGCCTTAAACCCAGGCTTGCAGCATAACGGTCTGTGCCTCAGTTTCCCCTGCAGCCCCTTGG
GATCCCTTTCCCTGGGTCAGATGAGAAAATGCCCAGTACATGTGGATACTTACTGAACCTGGCCTGGTGT
CCCTTCTCCCTCCTTAGCTGGATGCTGAAGCCAGTGACATCCTGAAGGAGCTTCAGGTGAAACTCAATAA
CGTCTTGGATGAGCTCAGCCGGGTGTTTGCTACCAGGTGGGAGCATCTCCAGGGCTGGGGGCAGTGGGGA
GGGAACAGCCTTCAGCCCTCTAAGCCTGGGGCATCTGGAGGCTCAGCAGATGTAGAGGTGAAGATTGCAG
GCTCAGAGTTAGACAAGGCTGAGTTCAAATCTTGGCTTTACTGCTTACTAGCTGTGTGGCCTTGGGTGAG
TCACTTAACTTCTCTGAGCCTTATTTTTTTTTTTATTGCTCTGTAAATTTGGGGTGATGACAATAATAGT
ACCTGGGTAGGATTTTGAGAATGAAATGAACAATGCGGGCAAATGTCTTATCCCTTTGCCTGGTTCTGCA
GTTACTATTTTCCACTCTTTTTTTTTTTTTGAGACGAAGTCTCGTTCTGTCACCAGGCTGGAGTGCAGTG
GCGCGATCTCGGCTCACTGCACCCTCCACTTCCTGGGTTCAAGTAGCTGGGACTACAGGTGCATGCCACC
ACACCCAGCTAATTTTTGTATTTTTAGTAGAGACAGGGTTTCACCATGTTGGCCAGGGTGGTCTCGATCT
CTTGACCTCGTGATCCGCCCGCCTTGGCCTCCCAAAGTGCTTGGATTAAAGGCGTGAGCCACCGCATCTG
GCCTGTTCTCCATTCTTGCTCGGTTATGTGCTGTGATGCAAATGATATGACCTCAAGGACCACAGATCAG
CATCTGTGTCCTCATCCAGCCCTTCCTGTGGTCTGAGCCCTTCCCTTCTGTGTCCCCAGCTTCCAGCCGC
ACATTGAAGAGTGTGTCAAACAGATGGGTGACATCCTTAGCCAGGTTAAGGGCACAGGCAATGTGCCAGC
CAGTGCCTGCAGCAGCGTGGCCCAGGACGCGGACAATGTGTTGCAGCCCATCATGGACCTGCTGGACAGC
AAGTGAGCTGGGCCAGGTGGACTGGGCTGGCCGGGGAGGGGCTGGGGCAGGGCCATAGAGACCCAGGGAC
TGGCTGGAGAGGGTGTGCAGGGAATGTTTGCTGGGTGGTCACTGGATAGTTGGATGGCTGAGTAGATGGT
GGCATGAAGGGGATGGAAAGTTGGGTGGGTAAATAATTGGAAGGATTGGTGAGTAAATATTTGTGTGGAT
GAATGAATGGTGGATGAATAGGTGACATGAGGGATGGGTAGGTGGGTGGACGCATGGATTAAAAGATAAA
TGTTTAGAAGGATAGAGGGAGGATAGATGTTTTAAAAAGATGGATGGGTGGATAGTTGGGTAAATATTTG
GATGGATTAATAAATGTTTTGATGCATAGATGGATTGATGCTTAGATGGATGGATGTCTGGCAGGCTGGC
TTGATGGATGGTTGGATGGACGATGGATGGATGGATGGATGGATGGATGGATGGATGGATGGATAAATGG
TTGGATATGTGGATAAATATTTGGATGGATGAATAAATGTTTTGATGCATAAATGGATGGACAGATGGGT
GGATGGATGAATAAATGGATGGGTGGGTAGATGGATGGATAGATAGGTGGATAAATATTTGGATGAATGA
ATAAATGTTTTGATGTATAGATGGATTGATGCATAGATGTCTGTCTGGCTGGCTGGATGGATGGATGGAT
GAATGGATGGATGGATAGGTCAATAGATATTTAGATCATTGAATAAATGTTTTAATGCATAAATGGATGG
ACAGATGGGTGGATGGATGAATGGATGGTTGGATGGGTGGGTGGGTAGATGGATGGATGCTGGATAGATA
TAGGTGGATAAATATTTGGATGGATAAATAAATGTTTTGATGCATAAATGGTTGGACAGATGGGGGGGTG
GGTGGATGAGTGGGTCGATGGATGGATGGATTAATAGGTGAGAAATATTTGGATGGATGAATAAATGTTT
TGATGCATAGATGGATGGACAGATGGATGAATGGACAGGTGGATGGATAGATAAATGGATAAGTGGATGG
ATGGGTAGAAGGATGCATGGAAGGATGGATGGATGAACAGATGGATGGATGGATGGATAGAAAAAGAAAT
AGAGAATTAAGGACCACTGGGGGAGGGATGGATTGGTGGGTGACTGGATCAGTTGGTGGATGGATCTTGG
TGGACTGCCTGTCTCCTTCAACCCCTATCCATCCAACCACAATCTCTTTGCTGTTTTCCCTTTCAAGTCT
GCCCTCCTCTGACCATTCCCCTCCTGTTCCTCTTGGGCATGGCCTTCTCCCTCATAGTCCCTGATCTCCA
TCCTTCCTGTTTCGGTTCATCCCCCACACTGTTCTTTCAAACATGAAAGTCTGGCTGTGTCTCCCTCTTG
AACACTCCATGGCTCCCCACTACCCCCATCCTGATAAAACCCAAGCCTTCCTCCCAGACATTGGGGCCCC
TTCCCATCTGGTCCCTGCTGACTAGTCCAACCACCACTCACTCTTCTCTTCATGCATCAGATATCATAGC
CCCATCAAACCACCCAGGGGTCCCTGTACAGGCTGTGGGCCCTCTTTCCTATCTGTGGAATGCCTTGCCC
ACCTGTTAAGGGAAGGTGATCTGTGGGTGGGGGCGAGCTGGGCCCTCTCTCAGACCTGCCCCTCGTCCCC
AGCCTGACCCTCTTTGCCAAAATCTGTGAGAAGACTGTGCTGAAGCGAGTGCTGAAGGAGCTGTGGAAGC
TGGTTATGAACACCATGGAGAAAACCATCGTCCTGCCGCCCCTCACTGACCAGACGGTGAGACCTGCAGG
GGGCCCGAGGGGACATTTAGGCCACCTCCCTGGCGAGAGCCCAGAAAACTTGGTGCCTAGAGGCTGGGGG
GTAAGAACAAAGGCATCCGGTCTCAGAGAGGTCATCCAGGCTCAAGGGCCATTCAAGGGTCATGGAAGCC
ACCAGAGGTCAGTGGGGGGCCATTCAGAGGTCAGAGAGTTCACACAGGGGTTAAAGATCATCGAAGAGTT
AAAGAGGTCATTCAGAGTCCATTGTATTTTCTCTGGGGTCAAAGACATCAGGTAGAGTCAAGAGACCACT
AAAGTCATAGAGGTCACATGTAGGTCAAAATAGCTTTCAAAGGTCAGAGGTCATCTAGAAAACAGGTCAA
TTTTGGGATCAAGGTTATCCTTGAGCCACGGAAGGCATAGACATTGGCCAGGCACCGTGGCTCACGCCTG
CAATCCCAGCACTTTGGGAGGCTCGAGGCGGGCAGATTGCTTGAGGTCAGGAGTTCGAGACCAGCCTGGG
CAACATGGTGAAATCTCGTCTCTACTAAAAATACAAAAATTAGCTGGGTGTGATCCTGTGATCCTGGCTT
CTTGGGAAACTGAGGCACGAAAACTGTTTGAACCTAAGAGGTAGAGGCTGCAGTGAGCTGAGATGGCGCC
ACTGCACACTCCAGCCTGGGCAACAGAACGAGACCCTTTCTCAAAAAAACAAAAAAAAAAAAGAGAAGGC
ATAGACATCAATGGGTTTTTCTAAGGGCTTTGGGGCTCATTCAAAAGCCAAAGAGGGGAGACACCATGGA
CTTCAGACATCAGAGATCAATAGACCAAGGGGCTCCTGGAAGTCAGCCAGAGGTCATGGAGGGCATCCAA
GGTCAGAGAGGTCAAGCAGAGATCATTGGCCATCCAGAGACCAGGACCGATAGACCAGAGGCCATAATCT
CTAGGGACCAAGGGGACCCAAACATCTGTAGAGGGATGTAGCTGGGCGTGGGTCCCTCCGGGAGGCATCA
AGAACCTGAGGACAGCAATGGCATGTGGGCTGTGGGCACATTAGGGAGTGGGGAGGTCTTTCCCAGGTGG
ACCACGGAGGCTTGGGGAGGGTGAGCGGGGGCCATAGATGGGCGACGATGGTCGGGGAGGCTCGGACTTC
CCATCCTTCCACCTCTTCTGGGCTGGGCTGGGGCAGGGGTCTTGCCCCTCCGTGCTGCCATCTCTGCCCC
CTCCCTGGCCTTCCCGGGAGACTCATGGTACCTTATTCCCCCCTCCCCCACCGCCCCGCCCCCATCACGG
ATGACAGATGATCGTAAGTAGAGTCCCGTCTGTCCGTCTGTCTGTCCGTCCTCTCTGTGTCTGGACTCGG
CCCACCGCGTCGCTGCACCCCTCTCCCCACCCACCCAGCGCCCCCTCCTCACCCACCATCCTCCCTCCCG
CCTAGATGTGCCTGTGTCTGTCTGGGGCGGGGCATGGGGAGGGGGCGGGGCTGGGAGGGAGGGAGACCAC
GCCCACCCTCCCTGTCCCACTGAAGTCCAGAGGAGCCCCACTACCCACTCCCAAACCCCAAGCCCAGCTC
CTCCTTCGCAGCGGACCTGCCGTCTAAGGGTAGAATCCAACCTTGCTCCCGTGCCCAGATGTCACCAGAC
GCCCTGAAGCTAGAAAGAGGATCAACCCCAGGATTCCTCTGACCTCCTCTTTGGGCTCTGAACTGTCAGC
AGCTTCCCAGAATGCAGGTCCTCACCATGAGGGTCTCTACTTCTGATTCCTCTGAAATTCCATGCTCCAG
ACCATTCCTTGGTACCCCACCACCCTGTCTACCCAACTCCCGCCATCCACACCGACACACACCAATCCCA
GACATACACCCAGGTGCCCAAGTGGGAGACTTTTGTCATGGCTTTGATGTCAGGTAGACTTGGGGTCAAA
TCCTAGCCCAGCCACTGTCTCTCTCTGTGACCTTGGGAAGTCACTTGACCTCCCAGCCTCTGTTTCCTTG
TCTGGAAAATGGGGCTCATTGCATCACCTCCCTGAGGGTAGTTCTAAGAACCAAATGGCATGATGCATTA
AAATACTCATCTCAGTATTGCCTGAACCCAGGAAGCAGAGGTTGCAGTGAGCCGAGATCGCGCCACTGCA
CTCTCAGTGACCGGCCCAGAGGCAGAGCTAATGGTGGTGGTGGTGGTGGTGGTGGTGGTGGTGATATGCA
GACATGTGTAAGAGCAGAGACAAAACAACTTCAGATTTTCCCTTTGTGGGCAAGACAAGGCTGTTACAAG
ATTTCACTTTGTTAAAAGTTTACAAGGGCTCTTACTTCCTGGTAGCTGCAGCCTGGGTCTTTGGCTATAA
CCACTTGGTGGCAGCATACCCAACCACAGGCAATGCATTTGTAGGCAGGGGGAATAAAGGAATTCGCATT
TAATGACTCCCCATATGCCAGGCCTTCTCATAGCTCCTGTTTCAGCTCACCTTCATGATAACCCTTTGAG
TGAGGTGGATATTATTATCCCCATTTTGCAGATGAGGAAAGCTGAGGCTCAGAGAGGAGTCACTTGTTCA
AGGTCATATAGTAAATATATGCCAGAATCAGGATTCAAATTCAGATCTGTCTGAATATGCAGCCCATTTG
CTTTCCTGTTAACCCAAGAACCAAGAGTCTCATGTGTACAGTAAATGTAAATGGATTATATTTTGGCAGT
GGCTTTTTTCCCTTCTTTCTGACTTTCTTTAGCTCTGTATCTGTATCTTTCTCTCTCTTTCCTGTCTCTG
CCTCTCACACCATTTAAGAGTGAGTCACTAAAGCGCATGCACGTGTGTGCACACACACAGACACACACAC
AAACACACTATCCCCATGCAACCCAGTCACCAGTACCCTGTGCAGTGTGGATTCCCCCATCCCCATGAAT
ATTCCTGTGAGCATCCCAGCTTCCCACCCTGCACGACTTGCCAGGTTGATCACCCTGTGACATTATCTCT
GCAGGGGAACCTCTTGAGAAAACATGGCAAGGGATTAGAAAAGGTAATGAGGGCCTCGTCTCACTGAGGC
CCTGAGCTCCAGTGTCTGGGCATGTGTGTGCATGTGTGTGCGTCTGTGCACCTGGGCAGGAAGGGCAGCT
GGGGGTCGTGGGTAGGAGATAGCTCTCCTAACCAAGTCCTGCAAAACCAGAAGTCAGAGATGAAGTGATT
CTCCAGGGACAGAACGTGCAGTGTGTGTGTCCACATGTGAACTAATGTGTGTGCACAAACAGCTCCCATC
CTCCCACAGCAATACCCACAGGGCTCATCTGAGCCCTGGGCTCAGCTGAAATTCTGTGCCTTCTTGAGGA
GCCTACACGCTAGATCACCACCCTGGACTGAGACGTGTTGTTTTTTACGTAGATTTCTTTTTGTTGCTGT
GCTAATGGGTGTTTGTTGTTAAGGAGATGGGAGCTACAGCAACACGCAAGCAGCTGTCTTGCTGTACACT
TGTGCAGAAGATATCGTTGTACATTTGTGTCTACAAATGGAGTCTTAGCTTTGTGAATGTGGGTCACAGG
GGTTCCGTACTCTCTCTGCCAGCCCTTTGCGGGGCACATTCAGTGTCCCAGGCACCCTATATGTGGATGT
GAATCCGTGTGGGGTCAAGAAGGGTGTGTGGGTTTTTGTGTGTGATATAATTGTGTACGTGCCTGCAAGT
GTTTAGGAGTGCACGTATGTAGTTTAGTGTATGGGTTCTTCAGCACTGAGTGAACTTACAGTATATGTTA
ATATGTTTTTTGTAGACTTGGGTGCTGGGATATAGGGGCAGCCCCTTAGGGAATGGAGGAGGAGACAGAT
TCCTAGCCCTGTCTCATCTCCCCACCTCCCCAGGTGCCCCATCCTGGGGCAGGCTGACTGTGGGATGAGG
CTGCTTCCAGCCTCCTGGGTACCCCGAGAGTGTGCTGGCAGTCAAGGAGTCTGACCTCTAAACCCCTGCT
AGGGGACCAGAGGAGGTGCTAGCTGGGGAGGGGGCCCACCATTAGACCCAGGACCTGGGGAGGAGCTCCT
GGGCTGGGAAGTGAGACTGTGCTTTGAGGGGGCAGGGAAGTCCCACTGAGTATCCTCCCAACAGTCACCA
GAACTCTGACCTCCACCTGATTCTCTCCACAGGGCAGGGTGAAATTGCCAAGCCACTCAGACGTAAGACC
GGCCTCTCACTGTCTGTCTGGCTCCCATCCCACTCCCCCCCACCCCACCCCTGTGCCCCCTTCCACCTCT
GTGGTTCGTGCCGTCCGAGGGCGTACTGTGAACCTGTCTGTTGGTCTCTTCTTCCTTTGTCTGTCTGGCT
TGCTCGTGGGGGGTGGTGGGTGGGGGGTTGGTGGGTAGGGCAGAGGGTACCCAGGAAGGCCCCTGAGCCC
TGCCAGACTACAGGTGGGTGCAGCAGGGGAAACTGAGGCCCAGTCCGCTGGACCCCTGAGTAACTGTGCC
TTGTAAGAACCTCAGGATCACAGACAAGAAATAAGAATAGTAACAGCCAAGATGATAATAGTAATACCTC
TAGGAATGACCAGATGGCCAGCTGGTAGACTATGAACATAATTCCAAATATTTCCACAGTAAATGGGCCC
CCTCCCCACCTAGCCTCACCCAGGACCCCCAGACCACCCCAGGACCCAGTAAGACAAGACTTGTTACCTC
AATACTCAGCCCCACCCTGTCCCCCAAGTTACCATGGGTATCCCTGTTAGCCACAGTGGGAATTGAGGCT
CAGAGCATTCAGGGACAAGCCTGGGGCCATCCAGCAGTGACAGAGGAGGCTAGACACATGCCTTCCAGGC
TGGGACCCCGTCTCCCTTAGCATTGCCCGTGACTTTGAGACCAAGTGATAATTAGCGAACACTGAGCAAA
GACTTGTTCAGTCCAGACTAAGCCACAAAGTGGATATAATTTTCCTCATCTAGATGGGAAAACTGAGGCT
CAGAGAGGTTAAGTGCCTTGCCCAGGGCCACACAGCAGGACTGCAATTTGAACCCAGGCCCATCTGGCTT
GTCAGAGCCATCACCACATCACCATCTTGCTAGTGTCTGTTTGGGGGTGGGGTGGAGGACGGATATGGAG
GTGTTTGTATAGAGGGAGGATTTTTCCTTTAAAAGGTCAGACTGGCTGGCAATGTGGCTCACGCCTGTAA
TCCCAGCACTTTGGGAGGCTGAGGTAGGAGGATCGATTGAGCCCAGGAATTCAAGACCAGCCTGGACAAC
ACAACAAGACCCCATCTCTATGAAAAAAATTTCAAAAACTAGCTGGATATGGTGGCTCACACGTGTAGTC
CCAGCTACTGGGGAGGCGGGAGGATTGCTTGAGCCCAGGAGTTTGAGGCTGCAATGAGCTATGATCATGC
CACTGCACTCCAGCCTGGGCAACAGAGGGAGACTCTGTCTCAAAAAAAAAAAAAAAGAAAAGAAAAATCA
GACTGAAATTCCTCCCAGATTACAGGGGAAATTTCCCTGCAGAGGGGATGCTCCTTGAGGAGGAGGAAGT
GTTTGACCAGAGCCATTTTCTGCAACCTCAGCCTAGCCTATTGTGTTCCCAAAGCTCCCTTTCCCTGGAA
ACCCTGCCCACACCCACAAACACCACCGTCTGAGGCTCCCCCAACCCACTGCCCCTCACAGTCTGGTCCC
TGTCTGTTTCTCCTGTTACACAAGCCTGGGTGGGGCTGAGAGGGTGGGGGACTATCCTCACAGGACCGGG
ATTTTCAGGGACAAGGTCACCACATCCTCCTCAGTTCTTCCTGGGGAATTCCAGACCTCTGTCCCTGGGA
CACAATTATTGCCCTGCCCAGAGCCTTTGGGCAAGAACCAAGATGGTCTCTGGGGCTGGAGTGTCAGCTG
TAGCACCCCTGCCCTCACCCAGCTGTATGTTGTTACCTGCAGGGAACCCAGATGATCTTCAATGCAGCCA
AGGAGCTGGGTCAGCTGTCCAAACTCAAGGTGAGGAGTGGGGATAGATTATGGCGTGAGGGACTGCCCCC
CAAACTCATGTGGCTTCCAGGTTGCCACCATCCTGGGGGAGGGGTCCCAGGATGATGACATGCATTACAG
GCTCAAAGGACAGAAACTCTACTCACTGAAGCACAAGATATTTGCTCCCCAGTGTGTCAGAAAGATGTGA
ATGGTTCAGCTTCAGGTACAGCTTGATCAAGAGGGCAAGTCATTTATTCAACCAATATTTTTTGAGCACC
TGCTGTGTACCAGTCACATATTGGGTGCTGGGATGATGAAAACATAGACCCTCAGCCCAGCTCCTCCAAG
TTGAACAGGGATGAGACCCATAGACACAGATTCCAAAGGTGGAGCCCACTCCACCCACATGGCCCCTCTC
CCCATCCATCCAGGATCACATGGTACGAGAAGAAGCCAAGAGCTTGACCCCAAAGCAGTGCGCGGTTGTT
GAGTTGGCCCTGGACACCATCAAGGTGAGAGGCCCTACCCCTCCCCATCCCAGGTGGGGGATGTAGAGGG
TGTGGCTGCTGTGGTCATGGACTTAGGCTCGCCTCGGACCCACATTAATTTGTTCAATTTATGGACACTC
CCTCGTTCCCCTGTCTGTGCCAGAGCTGGGCTGGCTAATGCTGGAGCCTTAAGGAGCCCCAGGTCGGGGA
AGACTGCTGGATACAGACATTCTCAGCCTTGTAGGATTAGGCTTGGGGTAGAGGGAAGGATGTGGGCTGG
GGGAGCCCAAAGAGTGAGAAGGTGTAGAGGGAAGGATGTAAAGGACCTCGTGCCAGGCATTTCTTGAGGT
GTAGAAGGGTAGAGGGAAGGATGGGGGCTGGGCGAGCCCAAGGTGGGAGAAGCCAGGCCTTCCTGGAAGA
CGGCATGTGAGAGGTGGGTTCTGAGGAATGCATAGGAGTTCACTAGGCAAAGCAAAGGAAGAGGTGAGAA
AGTAGTAGCGGGAAGAATTGGGAGTGGGGACAGCAGGGCTACCCAGCCCTATGAGTGGGTAGAGGTTCCC
ACCATCCTCACCTCTCCCCACCCTGTCCTCCCCAGCAATATTTCCACGCGGGTGGCGTGGGCCTCAAGAA
GACCTTCCTGGAGAAGAGCCCGGACCTGCAATCCTTGCGCTATGCCCTGTCGCTCTACACGCAGGCCACC
GACCTGCTAATCAAGACCTTTGTACAGACGCAATCGGCCCAGGGTAAGGGTACCCAGACCGCATCACCCT
TTCCCGCTCCGCGGAGACAGCCCCGCCCCAAGCCTGAACGGCTGCCATTGGCTCACTCCCCTGTGACGTC
ATGGATGGGGCGGAATTGACATCTCTGCACCAAGTCCAGGGACCAATGAGCGATCAAAGGTTCATGGGGG
CGGGGCCTCTGACGTCACAGGCCTGGAGGTGATCGTCAACCCCTGGGAAAATCTGTGATGTCCTGGGGCC
AGACAATGATTAACAGCTCTGGGATGGAATGTCTTTGACATCAAGGACCAGGGTAGGATTGATGACTCTT
AGGTGTGGAACCTGTGACGTCGTAGGGCCACAAGGTGATTGTCATCTCCTGGGAAAATCAATGGTGTCCC
AGGGCCAATGAATGATTGTCAGCTCCTGAGGGACCTGGACAGTCATGGGTCCAGGGAATCGTTGACAGCT
CTGGGAGACTTCAGGAGTGATTGACAGCTCAGGATGGGCAGGACCTCTGATGTCAAGGGGTCAAGGAATG
ATTGGCAGGTCCATGGGTGGGAACTTTGACGTCATGGTCCCAGAGAGTGGTTGAACTCCCCTGAGGAGGG
ACCTCTGTGATGTCATGGGGCTAAGGAATGATTGACAGCTCCGAGGGCGGGAGGGACCTCGACTTGGTGG
GGGAGGGTGGACGGAGAGGGAGTTCCCATTTCTCAAACTCTCTCTTTCTCTCTGTATTTTGCTTCGGTCT
CCTGTCCCTCTCTGAGCGCACACACACGGCAGCTGGGGCTGGATGGAGGGCAGGATTCCCTGGGGACCCC
CCCTTTTCAAAATTGGTCCCGACAAGGGGAAGAAGTTTGGCGGCTTGAACTCAGGAGGGGGAGGGAGAAT
CGCCATCGACACCCAGCCTGGGGCGAGGGGAGCAAAGCCCTGTGGCCTCCAACGGCCCCCTTCTCCCCCG
ATCCAGCCAGCCCAAGGGCCCTCCGTGGGCGCTGGGTCTCTGAGCTCAGCTTCCCTTCCAGTCCTGGCTG
CTTTTAGTGTCCCTGGTGTCCCTGGAGCAGGAGGCTGTCTGTCTCTCCGTCTGTCTGCAGCTGACGTTTC
TCTCTCTCTTGTTTTTCCTCCCTCTCACTCGCCGTCCCCTCTCCGCGCCTCCCCTGCCCCCTCCACACAC
ACTCCTCCATCCTCACCTCTCACTGGCTCGTCTCTCCCCGCTCCCCATGCTTCCCACCTCTCTCCCCCTT
CCACCCCCACCCCCAATTAGTCCATGGTGGAAAAGGTACTAGGTTTACCCTTAGTGAAGACATTTATCCT
GAGAAGGGTACGTGCCTCCGCCTCCCGCCCGCCGCCCGCGCCTGGTGCCCGGTGCCTGCGCCCCTCCTGG
CCCCGGCTAGTAAAAGGATCGTCCTTCTGCCTCAAGGGCCTGCAGGGCCGCCGCCCAGGCCTGAGCGGCT
TGGAGGGCCCAGGCGGGTGAGGGGGAAGGACGATTTGCTGGTTCTACATGGGGAAGAAGCGATTCCAGTG
GGAATCCCAGAATCCATTTCTCCTGATATGACATATTTTTTAAGCCAGATCCTGGGACCCCGGGACAGGC
ACCTGTCAGGTTAAGAGCTGGGCTCTGATTCCAGCCCAGCCACTCCACGGGTAGGCACCTTGGGCAAGAA
TCTGTCTCCCTGAGCCTCAGTTTTCTCATCTGTAAAATGGGGACAGTCAAGGACCCTACTTCATAGGATT
ATTGTTATTGATATTATTATTTAGAGACAAGGTCTCCTTCTGTCACCTAGGCTGGAATGCAGTGGTGCAA
TCACTGCTCACTGCAGCCTCCCTCCTGTGCTCAAGTGATCCTCCTGCCCCAGCCTCCCGAGGAGCTGGGA
CTACAGGCACACGTCACATGGCTAGCTAATTTTTAATTTTTTTTGTAGAGAGGTGGCCTCACTTTGTTGC
CCAGGCTGATCTCAGTGAATACTTATCAGGCTCTAAGGCTGAGCAGGACCTTGTGCCAGGCATTTCTTGA
AGAATCTGTGTTTGGTTTTGTTGTTGTTGTTTGAGACAGAGTCTCACTCTGTCACCCAGGCTGGAATGCA
GTGGGGAAATCTCAGCTCACTGCAACCTCCGTCCGCCTCCTGGTTTCAAGCGATTCTTGTGCCTCAGCCT
CCTGAGTAGCTGGGATTACAGGCCCGCACCACCACGCCTGGCTAATTTTTGTATTTTTAGTAGAGACGGG
GTTTTGCCATGTTGGCCAGGCTGGTCTTGAACTCTTGGCCTCAAGTGGTCCACCCATCTTGGCCTCGGCC
TTCCAAAGTGCTAGGATGACAGGTGTAATCCCAGCGCCTGGCCATTTTGTTTTTAATTTGAGGCAGACGC
TGTCACCCAGGCTGGAGTGCAATGGCGAAATCTCAGGTCACTGCAGCCGCCACCTCCTGGGCTATGTTGC
CCAGGCTGGTCTCGAACTCCTGGCCTCAAATGATCCTCCCACCTTGGCCTCCCAAAGTGCTGGGATTACA
GGCATGAGCCTCCATGCCTAGCCTTCTTGGGGAAACTGAAACAGATTTCTTTAACTGCCCCACGAGGGGT
ACATCATTCCACAGATGAGATACTGAGAGCCAAAGGAGATTCAACCACTTGTCCGAGGAACCTTCCCCTG
CAGGGGTCCCGGGACTTGGGGAGTGGGGAGTGGGGTTGGAAGAAGCACTGAGAGGCCCAGGAAAGACCCT
TCCCCGCGAAGCCAGCAAAGCACGGGATGGGGGCACCCAGCACTGCCTCCCTCCCCCCTTCTTACTAGCT
TTGGCCTTGGTCTGACCCCTCACCTGACCCACCACTGTCCAATTCCCCCTGGCACCCCCGGCACCCCAGT
CTGGCCCGGATTGAGCTTGCTATGAAGCATCGGCCTGGGGGAGGGCGGGGCGGCTGAGCATGTGACTGGT
GGCGGGGGGTGGGGGTGGAATGGGAGCCTGAGGCAACAAGGAAGACGGCCTGGGGCGTGCAGCAAAGACC
GGGGCCACATACCCCAGTACTGGGGTGATGGGGGTCTCCAGGCCCCTGTGCTCCCCAACCCTCAGTCACT
TCCTGTCCAGCGTGAGGTAACCCAATCAGCGTTTGACTGACCCAACCTCCCACCCTCACCTCTGACTTTC
CCCTTCTTCTCCCCGTGCATGGGGTGGTCCAGGAGTGGGGAGTTGCCTGGTGGGGGGTGGGGAGGGGTTG
GTGGAGGTGCTGGCGGTCACAGGTCCACAGGCCCCACAGGATGCCCTTTTGAGTCACAGAAGGCGGGACC
GGGAGAGATTCTTTTAAGCTGCCCCATCAGTGATCCCCATTGGCACCATGGAGGACACGGCTGGTGGCCC
CAAACAGAAGCCCACCACACCATGATTCAAGCAGACAAGGGAACTTTTTGACTCCATAATGGAAAAATTA
GGGGTTGAGGCCTGGCGCGGTGGTGCATGCCTGTAATCCCAGCACTTTGGGAGGCCAAGTTGGGCGGATC
ACTTGAAGTCAGGAGTTTGAGACCAGCCTGGCCAACATGGTGAAACCCCATCTCTACTAAAAATAACAAA
AATTAGCCAGGCGTGTTGGTGGATGCCTGTAATCCCAGCTACTCAGGAGGCTGAGGCAGGAGAATCGCTT
TAACCTGGGAGGCGGAGGTTGCAGTGAGCCGAGATGGTGCCATTACACTCCAGCCTGGTGACACAGCAAG
ACTCTATCTCAAAAACAAAAAAGAAGGCCAGGTGCGGTGGCTCATGCCTGTAATCCCAGCACTTTGGGAG
GCCGAGGCGGGCAGATCACCAGGTCAAGAGATCGAGACCATCCTGGCCAACATGGTGAAACCCCGTCTCA
AAACTACAAAAATTAGCTGGGTGTGGTGGTGTGCACCTGTAGTCCCAGCTACTCAGGAGGCTGAGGCAGG
AGAATCGCTTGAACCTGGGAGGCCGAGGTTGCAGTGAGCCGAGATTTCGCCATTGCACTCCAGCCTGGGC
GACAAAGCAAGACTCCATCTCAAAAAAAAGAAAAAAAAAAAAAAAAAAAAAAGAAACTTAAGATTCTGCA
CTAGAAGGGAAATAGGGGTAAGGAAAATAAAAGATGCCGCTATGAGCTGTCACATCAGGAGGGATGAGGA
TTCTGGGCCAGGTGATGCAATGAGGTGACCTGTGGGGCTGGGTCAGAGTTAACAAGGGCAAGAATGGGGT
GAGTGGGGAGAGGTAATAGTAACCTATTGGAAAGGGCCACTATTGGAAGGGGTGTTGCAGGTCACAAAAA
CCACTTTGATTATCTCAGAGTGAGGTGGGAGCCATGGAAGAGTTTGGTTTTGCTTTGTTTTGTTTTTTGA
GACAGTCTTGCTCTGTCACCCAGACTGAAGTGCAGTGGCGGGATCATAGCTCACTGCAGCCTCAACCAGC
TGGGATTAAGCGATCCTCCTGCCTCAGCCTCCTAGATAGCTGGGACTACAGGTGCATGCATCACCACACC
TGGCTAATTTTCTTTTCTCTCTCTTTTTTTTTTGAGACAGGGTCTCTCTCTGTCACCCAGGCTGGAGTGC
AGTGGTGCAATCTCAGCTCACTGCAACCTCTGCCTCCCAGGTTCAAGTGATTCTCCTGTCTCAGCTTCCC
AAGTAGCTGGGATTACAGGCGTGCACTACCACACCCGGCTAATTTTTGTATTTTTAGTAGAGACAGGGTT
TCGCCATGTTGGCCAGGATGGGCTTGAACTCTTGACCTCAAGTTATCTGCCCACCTCAGCCTCCCAAAAT
GCTAGGATTACAAGCATGAGCCTCCCAAAGTACTGGGGTTACAGGCATGAGCCACCACTGCCCGACCCAT
GGAAGAGTTTTGAGCAGAGGAGGGACATGATCTGACTTTTAAATGTTATTTATTTATTTTTTAATTTTCT
GAGACAGGGTCTTGCTCTGTCACCCAGGTTGAAGTGCAGTGGTGCGATCTCGTCTCACTGCAGCCTGGAC
CCTTCTGAGCTCAAGTGTTCCTCCCACCTCAGCCACCAGAGTAGCTGGGGCTACAAGCACGCCCCACCAT
GGCCACTAATTTTTGTATTTATTGTAGAGATGGGGTTTTGCCATGTTGCCCAGGCTAGTCTCAAACTCCT
GAGCTCAAGTGAGTTTGCCTCACCCTCCCAAAATGCTGGGATTACAGCTGTGAGCCACCGCGCCCTGCCG
ACATGATCTGACTTAATAGGATTTTTCTGGCTGGTATACTGAGAGTAGATGAAAGAGTGGAAGGAAGACC
AGCAAGGAGGCTACTGCAATAGTCCAGGCATGGGATGCTGGGGGCAGGTGTAGGATACAGGGAAAAGACA
TGGCTCTGCTTCTGGATGTATTTTGAAGGTAGAGCTGGCAGGATTTTCCAGTTTATGGGCACCTAGGGAG
GAAGTGAAGACAAAGAAGAGTGTCAAATATTGAGGTGCAGGGTCAGTTTGAGGACCCACGGAGGAGAAAA
TGACAAAAGGGGGTTGGAGGGAGTGACAACTATGTAGGATGAGAATCGAGTTGGTGACATTGTTGCAGCC
AAGAGGACAAACAGCCACAGGAGGCCAGGGTGCCAGATGCTGCCAAGATGTAATGTAACATGAAGACTGA
ATTTTGACAACATGGCAATCAATGGAATAAGAAAAAAGAAAAGAATTGCATTAGGCTCAGAGAGGTTAAG
CAATCTACCCAAGATCACACAGCAAGGCAGTAAAAGGCCAGAACCCAGGATTCTTGGCTTCCCAAGAGCA
CCTCCCATCCTGGCCCCCCATCAGTGCCTCCCTATCAGGGCTGATGACCTGGTGCCGCCAGCACAGGTGG
GTGGGAGGCCGTCAAGGTCCCTCCTGGTGGGTGGGAAAGAACAGAGCTCACGCTGACCATCCTCCCCTGC
CCTGCCCAGGCTTGGGTGTAGAAGACCCTGTGGGTGAAGTCTCTGTCCATGTTGAGCTGTTCACTCATCC
AGGAACTGGGGAACACAAGGTCACAGTGAAAGGTGAGTGGTCTGAGGTGGGAGGGACTTGCTAGGTTCTA
AAACAGAAACCAAACTCAAACTGGCTTAAGCAACACCCTTTTTTTGTTGTTGTTGTTTAATGGTCCATGA
AACGGAAACGTCCAAAGGTGGCCACCTTCAGGCATGGCTGGATCCAGCAGTGTAAACAGTTTTGTAAGAC
TGAATCCCACTTTCTCCATCTCTCAGCCCTGCTTTCCTTTTCTTGGCTTTTGTTGACTAGAACACAGATT
GGGCTGTTTTTGACATGGCTTAAATAAGACAGAATTAGGCCAGGCACGGTGGCTCATGCCTGTAATCCCA
GCACTTTGGGAGGCCAAAGCTGGCGGATCAGGCCAGGAGTTCAGACCAGCCTGGCCAACATGGTGAAACC
CTGTTTCTACTAAAAATTCAAAAAGTAGCTGGGTGTGGTGGCGTGTGCTTGTAATCCCAGCTACTCAGGA
GGCTGAGGTGGGAGGATCGCTTGAACCCGGGAGGTGGAGTTTGCAGTGAGCTGAGATTGCGCCACTGCAC
TCCAGCCTGGGTGACAGAGTGAGACTCCATGTAAACAAACAAACAAAAATGATAGAATTGGTTTTTCTCT
GGGATAACAGTTTAAGTAGCAACCAGCCTCGAGCTGATATGGAGGCTTCGCAGTGTTGGTGATCTGTGCT
GCTGCCATCTTTTGTTGCATTTTCATCCCTAATGTGTTGTTGTTGTTGTTTGTTTGTTTTAGAGGCCAGA
GTCTCACTCTGTTGCCCAGGTTGGAGCGCTGTGGTGCAATCACAGCACACTGCAGCCTCAAACTCCTGGG
CTCTAGCGATCCTCCTGCCTCAGCTTCCGGAGTCGCTGGGATTGCAGGCATGCAGCATCACACCTGGCTA
CTTTTTAAATTATATTTTTGTAGAGATGGTGTCTCACTATGGTATCCAGGCTGGTCTCAAACTCCTGGCC
TCAAGCAGTCCTCCCACCTGGGCCTCCCAAAGTGCTGGGATTCCAGACGTGAGCCACCATACCTGGACCC
AAATGTGTTTTTCCTCCACTATAGGGTCACAAAGAGCTCAGCCCCCACAAGCATTGCAGACTGCGGCTAG
AGGAAGGAGAAAGTTTGGGGTTCACTCCCTGCCTTTTAAGCCACAGTGGAGAAGTAGTACACGTGATTTT
TTTCTCTCACCTCATTGGCCAAAACATAGTCATATGACCACACTTAGCTTCATGGCAATGCTGGGAAATG
TAGTCTTTATTTCAGACAGGGTTTTTGGCGGCGGGCGAGGGGTGGGGTGTTTTTATTTTTTATTTTTTTT
AGACAGGATCTTGCTTTGTCACACAGGCTGGAGTGCAGTGGCACAATCTCAGCTCACTGCAACTGCCGCC
ACCCAGGCTCAAGCAATCCTCCTGCTTTAGCCCCACAAGTATGTGGGACTACAGGCGCGTGCCACCACGC
CCAGCTGATTTTTGTATTTTTCGTAGAGACAGGGTTTCACCATGTTGCCCAGGCTGGTCTCAAACTCCTG
GACTCAAGTGATCCACCCGCCTTGGCCTCCCGAAGTGGTGGGATTACAAGCGTGAGCCACTGCGCCCAGC
CTTGGGCAGGTTTTGAGAGCCACAAACATCTACTTTTTCCAACTGGGAGAACCTGACCTGTCTTACCCTC
TACTTCCTCCAACAGTGGTGGCTGCCAATGACCTCAAGTGGCAGACTTCTGGCATCTTCCGGCCGTTCAT
CGAGGTCAACATCATTGGGCCCCAGCTCAGCGACAAGAAACGCAAGTTTGCGACCAAATCCAAGAACAAT
AGCTGGGCTCCCAAGTACAATGAGAGCTTCCAGTTGTGAGTTTGGGATGCTTTGAAGAGCATGGGGGCAA
GGGAGGGGGCATCCGCCAGGTGAACCCCAAAGCCAGCTAGGCAGGAGGTATCTGGAATCTGGGCCCGGGA
ATGGGAGGGGCCACATTCTAGGTGGGGGTGCTGGAAAGGGAGACCCCTTAGTAGTGCAGGAATGACATCA
GGGAGGGGACACGAGGATGGAGGTCACCTTTGGGGGCTGGCAGATCCAAGGGGAGGCAATGAGTAGAGTG
GGGTAACTTCATGATGAAGACTTGCAGAGGGGAGGGAAAGTCTGGGCAGGAAGTGCATGGTGGGCAGGGC
CATTGCTGGGGCTGGAGACTCTGTCTGGTGAAGGAATGAGGGTGGCAGTGATGACGCTTATGAGCGGGGG
GCTGAGGGACCTGGGGACAGGACTGAGGTTGCATGGGGGTGGGGACTACTGGAGTCTACATTGAGCCTTG
TCAGGACCGTTGGGGCAGCCCATCTGGAGGAGTCGGCTCTGGGGACAGGAGTGGAGTGTAGATGGATCCC
AGGCTTGGGGGTGGGTGTCACGTGGGGTGGAGGTGGGGTCACATTGGATGGAGGTTCTTGGAGTTGGGAG
TGGGCCTAGGCTGCGGGTATCAGGATAGGGTCTAGAGGATGCTGATGATGTGGACAGCATAAAAAAGTCT
GCTCTGGCCGAGTGTGGTGGCTCACACCTGTCATCCCAGCACTCTGGGACGCCGAGGCAGACAGATCACC
TGAGGTCAGGAGTTCAAGACCAGCCTGGCCAACATGGTGAAACCCCGTCTCTACTAAAAATACAAAAAAA
AAAAAAAAAATTAGCCGGGTGTGGTGGCGCACGCCTGTGATCCCAGCTACTCGGGATGCTGAGGCAGAAG
AATCGCTTGAGTCCGGGAGGCGGAGGGTGCAGTGAGCCAAGATCGAGCCATTGCACTCCAGCCTGGGCAA
CAAAAGCGAAACTCCACCTCAAAAAAAAAAAAAAAGTCTGCTCTGGCCGAGCATGGTGGCTCACGCCTGT
AAGCTCAACACTTTGGGAGGCCAAGGTGGGCAGATAACTTGAGGCCAGGAGTTCGAGACCAGCCTGGCCA
ACATGGTGAAACTCCATCTCTATTAAAAATATAAAATATGGCTGGGTGTGGTGGCTCACACCTGTAATCC
CAGCACTTTGGGAGGCCAAGGCGGGCAGATCACGAGGTCAGGAGATCGAGACCATCCTGGCTAACACGGT
GAAACCCCGTCTCTACTAAAAATACAAAACCTAGCCAGGCGTGGTGGCGGGCGCCTGTAGTCCCAGCTAC
TCGGGAGGCTGAGGCAGGAGAATGGCGTGAACCCGGGAGGCAGAGCTTGCAGTGAGCCGAGATCGCACCA
CTGCACTCCAGCCTGGGCAACAGAGCAAGACTCCGTCTAAAAGAAATATATATTATATAATTATATATAA
AATATGTATTTATATTATATAATTATATATAAAATATGTATTTATATTATATTATATAATATATAATAAA
TATATATCATATAAATATATTTTTATATATTATATATAAATATATATATATTTATATACATATATAATAT
ATATTTATATTATATATAAAATTATATATAGTATATATTATATAGTATAAATAAAATTATATATAGTATA
TATTATATTATATATAAAAGTATATATATTATATATTTCATATATTATATATTGTATATAATATAGTATA
TACAAAAAATATTAGCTGTGTGTTGTGGTGGGCGCCTGTAATCCCAGCTACGCGGGAGGCTGAGACAGGA
GAATCGCTTGAACCCAGGAGGTGGAGGCTGCAATGACCCAAGATGGCGCTACTGCAGTCCAGCCTGGGCT
ACAGAGTGAGACTCAAAAAAATAAAAAATACAGAAATTAGCCGGGTGTGGTGGCAGGCGCTTGTAATCCC
AGCTACTCCAGAGGGTGAGGGGTACGAGAATCGCTTGAACCCAGGAGGCACAGGTTGCAGTGAGCCATGA
TTGTACCACGGCACTCCAGCCTGGGCGACAGAGCAAGACTCTGCCTCAAAAAAAAAAAAAAAAAAAAAGT
CTGTTCACGGGGGTAGATGGATGGTGGTGGTTAGCAAGGGTAGAGTTGGGGTCATCCGGGGTAGATCCTT
GGGTCGAGTGTCAGCCTCACAGAGCACTGGGGCAGGGCAATGTTGTTGGATGTTCTTAGGAGACAGAAGT
GCAGAAGAAAGTAGTCCCAGCTACCTGGGAGGCTGGGGCAGGAAGATCGCTTGAGCTCAGGAGTTTAAGG
CTGCAATGAGCTATGATTGTGCCACTGCACTCCAGCCTGGGTGACCGACATAGCGAGACCCTGTCTTTAA
AAAAACAAAAAACTATGGGCCAGGCGCGGTGGCTGACGCTTGTAATTCCAACACTTTGGGAGGCCAAGGC
AGTGGATCACCTGAGGTTAGGAGTTCGAGACCAGCGTGGCCAACATGGTGAGACCCCCCCCCCCGCCACC
CCCATCTGTACTAAAAACACAAAAAATCAGCCCGGTGTGGTGGTGCATGCCTGTAATCCCAGCTACTAGG
GAGGCTGAGGCAGAAGAATCGTTTGAACCCGGGAGACAGAGGTTGCAGTGAGCCGCGATTGTGCCACTGC
ACTCCAGCCTGGGTGACAGAGGGAGATGCCCACTCAAAAAATAAAAATAAATAAAAATAAATAAAAATGC
ACAGGGGTAAGGGTAAATCAGAGCTGGTTAGCTGGTATTATCTAGGGGGCCTGGGAGCTCTTATGGGACA
AGATTGAAGCCAGTTCCATGTCGTGTCAGCACAGGGCTCTTGGTCAGAAGGTTTTGGGGCGATTGGCCCC
TTTAGGGGATACTCAGGAGGAGAAGCATGGTCTAGTGGGAGGTCAGATGTCAGCCAGGGTGGGCCGGAGT
ACTTCAGGGATAGAGGTACAACCTGGGAGGTGGACAAGAGGGAGAAGCTGGGCGTGACCCTGTAGGAGGG
GCTCTTATCCAGCTGGCGTCAGGGCAGGTCACTGGGACTGCTTAGGGGCAGGAGCACAGGTGAGGGGCAT
GCGGGAGGCGGTGTCACCCATGGTGAGGCACCAGGGCAGGCTCATGTCTGGAGGGCATCTTGGAAGTAAG
AGGGGTGGGCTGGGGGTGTGGGGGGCTGGGGAAAGGGCCACGTCGTGGGTCTAGAGGAGAGCCTGGTTAA
AGAGCTTCTGGGTGGGACATTAGGGGCAGGGCCGTGCGGGCAGCAGTTCAGCCGGCAGAGGGCACAGGCG
CGGGTATCTCAGGGTGGGAGCATGGCCTTGCAGGGCGGTGTCTGAGGGGCGGGAGCTGGATGTAAAACTA
TGCGGTCATTTTTCGGTGGGTGGAAAAATGGGAAGAACAGGCGTGGGCGTGGCCCAGCTCGGGGTCAGCT
GCTGTGGGCGCGGCCAAGCACAGGGTTGTAGGTCAAGTAACGGGGCAGGGGCGGGGCTAGCGTAGCGTGG
GAGGTGACAGGTGTGGGCACCCCGGTGTGGGCGTGGCCCGCAAATGCGGTGGGGACGGGGCCGTGGGCGG
GGCATCACAGTAGGTGTGGCCTGTCTCACGTTCCGCCCCTCCCCACGCAGCACGCTGAGCGCCGACGCGG
GTCCCGAGTGCTATGAGCTGCAGGTGTGCGTCAAGGACTACTGCTTCGCGCGCGAGGACCGCACGGTGGG
GCTGGCCGTGCTGCAGCTGCGTGAGCTGGCCCAGCGCGGGAGCGCCGCCTGCTGGCTGCCGCTCGGCCGC
CGCATCCACATGGACGACACGGGCCTCACGGTGCTGCGAATCCTCTCGCAGCGCAGCAACGACGAGGTGG
CCAAGGAGTTCGTGAAGCTCAAGTCGGACACGCGCTCCGCCGAGGAGGGCGGTGCCGCGCCTGCGCCTTA
GCGCGGGCGGTCGGCCGAGCGGCACTGCGCCTGCGCGGAGGGCGCTGGGCGGGGAGGGACGGGGCTTGCG
CCTTGGTGGGACCTCCCCAGGGGCGGGGCTCGGGGGGCTCCACGCCAAGGGTGGGCTGCGCCTACGCCCT
TGACTCAGCTTTCCCTTTTGGGGAATTAGGAATGGAGGATGCCCCGCCCTCTCGGGAGGCCACGCCCAAG
GGCGCGACGAAGGAAGGAGCCACATCCCCAACTTGAGGCCACGCCCCCAGCACCTAGGGGGCATTTTGAG
CTGGGATGGGGGAAACCTCGTCCCTATGGAGGAGGCCACATCCCGGGGCTCTGGTACCGGGAGGCACCAC
CTCATGTCCCCTGGAAAAGCCATAAGATGGGACCCAGACCCCTGGGACCCCAGACCAATTGCCAAGTATG
GAAATCTCAGCTCCCTCGAGGGGGGGCCCTGGGCAAGGGGTAGGGCTCTCTGGAGCGCCCCTCTAGGTGG
CCTGGGGACTGGAGGGACCAGGATGCTGGTTGGAGGGCCCCGGAATACCGGAGTCCCTTTAGATATTTGT
GCAAAAAATAAATGGGGGGAGGGGGGAGGATGGGATTTCAAAAGCACATGCGCCCTTGGGCGCCCAAACC
CTGGGGGCCGAGGGGACGGCTCTGGTTCCCCACGCTGCCCCTACTTCCCTTTGGGAGTTTGCCTCTCCCT
CTCCCCCAACAAACCCAGTCCTCATATCATAGAGTTCAACACACCCATTTGACAGATGGCAAAACTGAGG
CTTAAAGAGCTGCTTGAGACTTGGCCAAGGTTCCAGGTGCCATACCCTCTGTGCCCCTCCCTTAGGCCTG
TGTGCCCCATGGAAGGGTGGGCTGAGATCGGGATGACCTGACACAGCTCCCTATTGCTGCTAATTCCCCC
TCGGCCTCCTCCAAGGGGTGGGAATTCCAGGCCAAGACCCCTACTTCGCCTTTCCTTCTCCGGCTGCCAA
GCAGGACCTTTGCCCTCAGCCCTTTCTCCTGGGATCTCCATGGGGGATGCCATGAGGGCCTCCCACCACA
AAAGAGAATTTGGGATCCCCTGGTCCCAGGTTTCTCCATCCCTTCTTCCTTTTCCAGAATTTTCCAAATA
GGAAAGAACAGAAGGAGACCAGAAACTCTAGGGGGGAGAAAGAGAATGAGAGAAAGAGAATGAGAGAGAG
AGAAACACAAACACAGTGACACAGTGAGAGCTTAGTCTCCAAGAGCCTATTCATTGATTCAAACACCCAA
GCCACAGGATACCTCAGATGGCCCTCTTGCCAGCTGGAAGCTCTTTCTCCAATGAGCAAAGTTACAGTGA
CCTGGCTGGAGTTACCTGGTGCACATAGGACCTTAGGGGAAAGTTCAGCGTGGACTACACTTGCTCTGGG
ATCTGCTTTTCCACATGTGTGTATGGCACGCCTTTTTCTGCTGGATTGGGAAGGACAAGATTTTGCTGTG
CTAGGGAGAAATGAAAACGGGGTGAGCTGAGTAGCTGGGTTTCTGGAGGATAGAACATCAGATGGGGAGG
CTTTCCGAGGTGAAGAATGAGAGGGAACCACTTACTAGAGAGAAAAGAGCTCCAGGCCTGGGGAACAGCA
CGTGCGAAGGCCAGGAGAGAAGAACTGTTGAAACAACGAGAAGGGTGGCACGGCTGGAGCTGAGCCAGCA
AGGGGGATCGTGAGGAGCCTTGGGGTTGGGGAGATCTGCAGAAGCATCAGACCAGGCAGGGCCTCGTACG
CAGTCCTGAGGAGTTTTACTTTTATTCTAAGACAGTTGGGGAGCTCCAGGAGCTGTTTTAAGTTGGGGAG
AGACTGGATTCCAGCCTGCAAAAGCTGTTTTGTGAAGACTAAAACCAGTGAGGAGAGGTGGAGGTGCTTT
GGGGACACTGAAATGGATTCTTGGAAAGATTCTGAAGGCTGTGTTGAAAAGACACCTATAGCTGTGGGGA
CATGACTATAATCCCAGCATTTGGGGAGACCGAGGCTGGCAGATCACTTAAGGTCAGGAGTTTGAGACCA
GCCTGGCCAACATGGCGAAACCCCATCTCTGCTAAAAATACAAAAATTAGCTGGGTGCAGTGGTGCATGC
CTGTAGTCCCAGCTACTCAGGAGACTGAGGCGGGAGAATTGTTTGAACCCTGGAGGCAGAGGTTGTAGTG
AGTCGTGATCACACAACTGCACTCCAGCCTGGGCAACAGAACAATACTCCATTCCCTCCCCTCTACCCCA
CCAAAAAAAAAAAAAAATCCTGCCCTTAGATGAGCTCTAGGGCTGCTGAGTACAGTTGTCCCAGTTGCAC
AGTGCCCAAGGGTTTGGCATTGCTAAGAAGGCCACGTGCAAATCCTAGATATTGAGTGTTGTATGTTTGT
GACGTTGGTTTCCCGACATGTGAATGGCCCAAGTGTCTGGAAGAAGTGGCGCCACTTTCTAATTTGCTTG
GAGATGTTGCATGTCCCTTAAATTCAGACAGGTGCAGGTAACTGGAGGTTCTGAACCAAAGGTTAAAATG
CAAATTCTCATACAGGGTTGGGAAGTTGTAGCCAGGGATAAGCTTATGTGACTGTTATATGGACTGAGGA
GCAGATGTGAATTTCGAACCATGACATGGCTGAGGGTAGGGGTCGGGTGGATGGATGATTCAGGGTTGTA
ACCCATAGAGCCCAAAGGGGAAGTGATCTGTGACCTGGGGTGAGGGTGATCTGGAAGATTTTTGGATGGC
TGGAAAGAAATGGGGAAGTCGAGCTGCCTGAGAGAGCCAAGTTATTTCCCAAAAGATTCCTTAGGAGTCT
TTCTGTTCAAGACCTCCGTGTGTGTGTGTGTGTGTTTAGGGTTCCCCAGCAATGGCCCAGGCATGTGAAG
GAAACAAGCTTCTTCAGGGAATATTTGTTGAATGAGTTTTCCTGACTCCCAGGCTAGAACTGTTTTTGCA
ATTTCCACCCTCTTTTCTTTCCCCCAGAGAACTCCTATTCGTCCTTCAAAACCCATCACGGAAACCCCTC
TTGGAGAAAACCCTCCTTCCTTCCCCTCAGGACTTTCCCAGCCACCGTCTCTCCTCCAGTCCAGCCTGAT
GCCATGGGACTGGGGGTTTCTCTGTCCAGCTCTGTTTCTCCCAGACTGGGGTCTGAGGACTCTCAGGACC
CCCAACTTTACCTAGCACAGGCTGGGCACAAGTGGGTGACAGGGAGTCTACGCCTAGTGGAATTATGTAT
TGGGGCAGGGTCAGTGTGAGAATACACATCCGCATGCATGTCTGTCCATGTCTGTCCGTACCAACCTTCC
CCTTCCACACGGACCTGGGCACATAGGAGGTGTCTGAGCCTGACACATGGGACAGAGAGTGGACATGGCT
GAGACACGGACAGAGAAAAGACAAGGAGTCCAGGGGGCTGAAAGCCTTTTGAAATCAGGAAGTTCCTGTA
TTGGCAGAACAAAGCCCAGAGAGGAGCAGGGCTTTCCTCAACGCCACCCAGCAAGTGGACACAGAGCCCG
GCCTTGGATGACACCTCCAGGGTTCTGAACCCTGGACCTCGCTTTATGCAAGGAGCTGGCCCCACATTTC
CATGAATCGGGGAAACAGCACAAGAAGGTTGGCCTGTGGCAGGGCAAGGGTTAAAGGGGTGACATTGAGG
GATGCCTCAGAGTCAAAGTCCCCTGACCAAGAGGAATAGAGTAGAAAACACAGAGACAGAGGGTGAGATC
ACGCCCCGATGAGGACGGAGAGAGACAGAGATGGAGAGAGACATAGAGGTGGAAATATACAGAGAAAGAT
AAATGCAGAGACCAAGGCAGGGAGTGTCGGGGGAAGTAAAGAGGGTGTCCTGAAGAAAGAAGGATCTGTT
CACTCTTACCAGTCTGTCCTCGAATGATTTGCATAAAATGAGGAGGTGCCTGTCCACACCCCCAATTCCT
CTCTCAGGCCCCAGAGCCTGAGACCTCACCATGCCCCCATCAGAGATGCAAAAAACTAAACACCCAACTA
GAAATCCTTGGGACCTCTCTCGGCTGGGATCTCAGAGCCTTTCTGTCCCCTACCCCTACCCCATGTGCTG
TCGATTTTGCAGATGGGGACAACCTGGGGCCTCCCGGAACTCTGCCACCCTGGGGAAGTTGGGGGAGGGC
CTTAGTCCCGGATCACAACCCCGTCTGCTCCCCAGAATCCTTTCCTAAGAATCGTTGAGGACCAAAGTTG
TCTTTGCTGACACGTGTTGCTTTTCTCTTTGCCTTTTATTGTTTCAGAGAAAAATCAAGTTGACTGTGTC
AAGTAACACCCCACCCCTTACCCCCGTCCAGCCATAGTGGCTCTCTGGAGACACAGGTCACAGGCGGAGG
GTCCCCTGATCATCCCCAACCACACAGCCAGGGGGACTTGACCCCTGTCCACCCCTGTCTCGTGCTCCCT
CAGACCCCCACAAACCGGCCAAGCAGTCCGGGGAGGCTTCCCCTCCACACAACTCTTAGCATGTGATTGC
AGATGTGAAATCAAAACGTTGTTTGTTTTTTGTTTTGTTTTGATTCTACCCCGTCGGTCCAGTGTCTGCA
CAGACGCCTTCATTTCTCTGTAAATATGTGACTTGGAACAAATGTTTAACACAAACGAGAAGTGGTCATG
AATGCATGGTGTTGAGATGTTTTGCACTATTCTGACTTTTTGGTCTCTGTAAAAATATTTTATTAACAGC
AGACATTAAAAAAAGAAAAACCACACACAGCCTTGGACACGTGGTTGCCTCCTCCTTGCATTCCTTTATC
AGCAAAAAAACAAAAACAAAAACCTCACGTACGATTAGCACCTACTAGGTAGGCGCAGACTCAGTAGAAC
ACACTGCTTGCCCATGAACGCGGCTCATCCGCAACTGGAGTCATCCCCATTTTACAGAGAAGGAAACTGA
AGTACAGAGAGACAAGATCTTGCTCAGATCACATAGCCAGGAAGAGATGGAGCTGGGACTTGAACTCAGA
ACATTTGCAACCATTGCTTTTTTTTTTTTTTTTTGAGATGAAGTCTCTGTTGCCCAGGCTGGAATGCAGT
GGTGCAATCTCAGCTCACTGCAATCTCCACCTCCCAGATTCAAGCGATTCTCCTGCCTTAGCCTCCTGAG
TAGCTGGGATTACAGGCGCCCGCCACCTTGCCTGGCTAATTTTTGTATTTTTAGTAGAGATAGGATTTCA
CCATGTTGGCCAGGCTGGTCGCGAACCCCTGACCTCAGGTGATCCACTCCCTCGGCCTCCCAAAGTGCTG
GGATTACAGGCGTGAGCCACCGCACCCGGCCATCTGTTGCCCTTAAGGAAGTTAAGATACAAACACTAGG
AATACATGCCCTTAATTCAACCCACACCCCCATGCTTGTTTGGTTTTGTTGGGTTTTTTGGGATCTTCTT
GTTTGTTTGTTTTTGAGACAGGGTCTTGCTCTGTCACCCAGGATGGAGTTCAGTGTCACAATCTCAACTC
ACTGCATCCTCAACCTCCCAGGCTCAGGTGATCCTCCCACCTCAGCCTCCTGAGTAGTGTGCACCACCAC
ACCTGGCTAATTTGTGTATTTTTTGTAGTGATAGGATGTCGCCATGTTGCCCAGACTGCTCTCGAACTCC
TCACTTTGGGCAATCCTCTCTCCTCAGCCCTCCAAAGTGTTGGGATTACAGGCGTGAGCCACTGTGCCTG
GCCATGTCTGTTTTAAATGTAGCAGCTGGGGCCAGGCGCAGTGGCTCACGCCTGTAATCCCAGCACTTTA
GGAGGCCGAGGCGGGTGGATCACGAGGTCAGGAGATCGAGACCATCCTGGCTAACACGGTGAAACCCCGT
CTCTACTAAAAGTACCAAAAATTAGCCGGGCACGGTGGCAGGCACCTGTAGTTCCAGCTACTCGGGAGGC
TGAGGCAGGAGAATGGTGTGAACTGGGGAGGCGGAGCTTGCAGTGAGCCAAGATCACGCCACTGCATTCC
AGCCTGGGTGACGGAGCGAGACTCCATCTCAAAAAAAAAAAAAGAAAAAAAAGTAGCAGCTGGGAATCTC
ATTTCATAAACTCAGATGTGTGAGGAATGAAACTCCATTTTCCAACAGCTGAATTTAATTTCTGTTTTGA
GTCTCCATCCCACGAGGAAGCAGAAATGGAATCTCATTCAGCCCAGTTTCCTCGGTAAATTCCGAAATTC
CCCTCCTTATGTCTGGGGTTATTTCTACATTTGAGCGGACAGGACGGGAGCTGCGGGATCCACCACCAGG
AGTGGGTTCAGTGAGTGCTTTTGGTTGCAAGTAACAGACAGCAGGACTAAAAGTGGGGCCAGAAAGTGGC
TTACACCAGGAGTTATCCAGCACGCAGCAATCCCGCTCCTGGGGGACTTTTCTCCTTTGAGAAACTTGCA
CGTGGGGGTGTGGTATCTTCTCAAAGAATAGCATATAGCTGGGAAAAGGGGACAAACCGCAGCTGTCTGC
ATGATGTCTGCAGAGGCAGCCTGCAGGTCCTTGAGTGCCCCTGCTCATTAGCTGTTTCTGTACCCAAGTG
CATAGGCTACGCACTGTGACTGATGTGGATCTATTTGCCCCTCGCTACAAAAGGGTCTCAAGTCTTGTCC
TTCAGTGTGTGGGACCCATCGTGTTACCAATGGGGCTTGGGGACTGAGGCTCAAGACTGCTTGCTGGGAA
ATGTAACCCCTTTGTTCTGGACAGGTAGAAGGAGAGGAGAATGGTCATCCAGGCCTAATGTAAAGTTTCT
GCAACCC.

In various embodiments, a UNC13A transcript is a pre-miRNA UNC13A transcript. In various embodiments, a UNC13A pre-miRNA transcript comprises a sequence provided as SEQ ID NO: 9595. NCBI Reference Sequence NC_000019.10 Reference GRCh38.p13 Primary Assembly is SEQ ID NO: 9595.

### UNC13A Oligonucleotides Targeting Regions of the UNC13A Transcript

In various embodiments, UNC13A AON disclosed herein are complementary to specific regions of UNC13A transcripts (for example, a UNC13A pre-mRNA) comprising a sequence that shares at least 90% (e.g., 90%, 91%, 92%, 93%, 94%, 95%, 96%, 97%, 98%, 99%, or 100%) identity to any one of SEQ ID NOs: 5057-5065. In some embodiments, a UNC13A AON comprises a sequence that is complementary to a specific region of the UNC13A transcript comprising a sequence that shares at least 90% (e.g., 90%, 91%, 92%, 93%, 94%, 95%, 96%, 97%, 98%, 99%, or 100%) identity to any one of SEQ ID NOs: 9587-9595. In some embodiments, a UNC13A AON comprises a sequence that is at least 85% complementary to a specific region of the UNC13A transcript. In some embodiments, a UNC13A AON comprises a sequence that is at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% complementary to a specific region of the UNC13A transcript. In some embodiments, a UNC13A AON comprises a sequence that is 90 to 99% complementary to a specific region of the UNC13A transcript. In some embodiments, a UNC13A AON comprises a sequence that is 90 to 95% complementary to a specific region of the UNC13A transcript. In some embodiments, a UNC13A AON comprises a sequence that is 95 to 99% complementary to a specific region of the UNC13A transcript.

In some embodiments, the UNC13A AON (e.g., UNC13A AON) has a segment that has, at most, 7 linked nucleosides. In some embodiments, the UNC13A AON has a segment that has, at most, 6, 5, 4, 3, or 2 linked nucleosides. The segments of the UNC13A AON may be separated from other segments of the UNC13A AON through a spacer. The segment of the UNC13A AON is complementary to a specific region of the UNC13A transcript (for example, a UNC13A transcript) comprising a sequence that shares at least 90% (e.g., 90%, 91%, 92%, 93%, 94%, 95%, 96%, 97%, 98%, 99%, or 100%) identity to any one of SEQ ID NOs: 9587-9593 or an UNC13A pre-mRNA transcript transcribed from SEQ ID NO: 9594 or 9595.

### UNC13A Oligonucleotide Variants

In various embodiments, UNC13A AONs include different variants, hereafter referred to as UNC13A AON variants. A UNC13A AON variant may be an oligonucleotide sequence of 5 to 100 nucleobases in length, for example, 10 to 40 nucleobases in length, for example, 14 to 40 nucleobases in length, 10 to 30 nucleobases in length, for example, 14 to 30 nucleobases in length, for example, 16 to 28 nucleobases in length, for example, 19 to 23 nucleobases in length, for example, 21 to 23 nucleobases in length, for example, or 18, 19, 20, 21, 22, 23, 24, 25, 26, 27, 28, 29, or 30 nucleobases in length. A UNC13A AON variant may be an oligonucleotide sequence complementary to a portion of a UNC13A pre-mRNA sequence or a UNC13A gene sequence.

In various embodiments, a UNC13A AON variant represents a modified version of a corresponding UNC13A parent oligonucleotide that includes a nucleobase sequence selected from any one of SEQ ID NOs: 4531-5794. In some embodiments, a UNC13A AON variant includes a nucleobase sequence that represents a shortened version of a nucleobase sequence of a UNC13A AON selected from any one of SEQ ID NOs: 4531-5794. As one example, if a UNC13A parent oligonucleotide includes a 25mer (e.g., 25 oligonucleotide units in length) a variant (e.g., a UNC13A variant) may include a shorter version (e.g., 15mer, 17mer, 19mer, 21mer, or 23mer) of the 25mer UNC13A parent oligonucleotide. In one embodiment, a nucleobase sequence of a UNC13A AON variant differs from a corresponding nucleobase sequence of a UNC13A parent oligonucleotide in that 1, 2, 3, 4, 5, or 6 oligonucleotide units are removed from one or both of the 3′ and 5′ ends of the nucleobase sequence of the UNC13A parent oligonucleotide. In one embodiment, the corresponding UNC13A AON variant may include a 23mer where two oligonucleotide units were removed from one of the 3′ or 5′ end of a 25mer included in the UNC13A parent oligonucleotide. In one embodiment, the corresponding UNC13A AON variant may include a 23mer where one nucleotide base is removed from each of the 3′ and 5′ ends of the 25mer included in the UNC13A parent oligonucleotide. In one embodiment, the corresponding UNC13A AON variant may include a 21mer where two oligonucleotide units are removed from each of the 3′ and 5′ ends of the 25mer included in the UNC13A parent oligonucleotide. In one embodiment, the corresponding UNC13A AON variant may include a 21mer where four oligonucleotide units are removed from either the 3′ or 5′ end of the 25mer included in the UNC13A parent oligonucleotide. In one embodiment, the corresponding UNC13A AON variant may include a 19mer where three oligonucleotide units are removed from each of the 3′ and 5′ ends of the 25mer included in the UNC13A parent oligonucleotide. In one embodiment, the corresponding UNC13A AON variant may include a 19mer where six oligonucleotide units are removed from either the 3′ or 5′ end of the 25mer included in the UNC13A parent oligonucleotide.

Example sequences of UNC13A AON variants are shown below in Table 10.

TABLE 10
Example UNC13A Oligonucleotide Variant Sequences

| | AON Sequence* | | Target Sequence |
|---|---|---|---|
| SEQ ID NO: | (5′ → 3′) | SEQ ID NO: | (5′ → 3′) |
| 7059 | GGAGGGGCCGAGCAATGACCCTC | 8323 | GAGGGTCATTGCTCGGCCCCTCC |
| 7060 | GGGAGGGGCCGAGCAATGACCCT | 8324 | AGGGTCATTGCTCGGCCCCTCCC |
| 7061 | TGGGAGGGGCCGAGCAATGACCC | 8325 | GGGTCATTGCTCGGCCCCTCCCA |
| 7062 | ATGGGAGGGGCCGAGCAATGACC | 8326 | GGTCATTGCTCGGCCCCTCCCAT |
| 7063 | CATGGGAGGGGCCGAGCAATGAC | 8327 | GTCATTGCTCGGCCCCTCCCATG |
| 7064 | GCATGGGAGGGGCCGAGCAATGA | 8328 | TCATTGCTCGGCCCCTCCCATGC |
| 7065 | GGCATGGGAGGGGCCGAGCAATG | 8329 | CATTGCTCGGCCCCTCCCATGCC |
| 7066 | TGGCATGGGAGGGGCCGAGCAAT | 8330 | ATTGCTCGGCCCCTCCCATGCCA |
| 7067 | GTGGCATGGGAGGGGCCGAGCAA | 8331 | TTGCTCGGCCCCTCCCATGCCAC |
| 7068 | AGTGGCATGGGAGGGGCCGAGCA | 8332 | TGCTCGGCCCCTCCCATGCCACT |
| 7069 | AAGTGGCATGGGAGGGGCCGAGC | 8333 | GCTCGGCCCCTCCCATGCCACTT |
| 7070 | GAAGTGGCATGGGAGGGGCCGAG | 8334 | CTCGGCCCCTCCCATGCCACTTC |
| 7071 | GGAAGTGGCATGGGAGGGGCCGA | 8335 | TCGGCCCCTCCCATGCCACTTCC |
| 7072 | TGGAAGTGGCATGGGAGGGGCCG | 8336 | CGGCCCCTCCCATGCCACTTCCA |
| 7073 | GTGGAAGTGGCATGGGAGGGGCC | 8337 | GGCCCCTCCCATGCCACTTCCAC |
| 7074 | AGTGGAAGTGGCATGGGAGGGGC | 8338 | GCCCCTCCCATGCCACTTCCACT |
| 7075 | GAGTGGAAGTGGCATGGGAGGGG | 8339 | CCCCTCCCATGCCACTTCCACTC |
| 7076 | TGAGTGGAAGTGGCATGGGAGGG | 8340 | CCCTCCCATGCCACTTCCACTCA |
| 7077 | GTGAGTGGAAGTGGCATGGGAGG | 8341 | CCTCCCATGCCACTTCCACTCAC |
| 7078 | GGTGAGTGGAAGTGGCATGGGAG | 8342 | CTCCCATGCCACTTCCACTCACC |
| 7079 | TGGTGAGTGGAAGTGGCATGGGA | 8343 | TCCCATGCCACTTCCACTCACCA |
| 7080 | ATGGTGAGTGGAAGTGGCATGGG | 8344 | CCCATGCCACTTCCACTCACCAT |
| 7081 | AATGGTGAGTGGAAGTGGCATGG | 8345 | CCATGCCACTTCCACTCACCATT |
| 7082 | GAATGGTGAGTGGAAGTGGCATG | 8346 | CATGCCACTTCCACTCACCATTC |
| 7083 | GGAATGGTGAGTGGAAGTGGCAT | 8347 | ATGCCACTTCCACTCACCATTCC |
| 7084 | AGGAATGGTGAGTGGAAGTGGCA | 8348 | TGCCACTTCCACTCACCATTCCT |
| 7085 | CAGGAATGGTGAGTGGAAGTGGC | 8349 | GCCACTTCCACTCACCATTCCTG |
| 7086 | GCAGGAATGGTGAGTGGAAGTGG | 8350 | CCACTTCCACTCACCATTCCTGC |
| 7087 | GGCAGGAATGGTGAGTGGAAGTG | 8351 | CACTTCCACTCACCATTCCTGCC |
| 7088 | AGGCAGGAATGGTGAGTGGAAGT | 8352 | ACTTCCACTCACCATTCCTGCCT |
| 7089 | CAGGCAGGAATGGTGAGTGGAAG | 8353 | CTTCCACTCACCATTCCTGCCTG |
| 7090 | GCAGGCAGGAATGGTGAGTGGAA | 8354 | TTCCACTCACCATTCCTGCCTGC |
| 7091 | GGCAGGCAGGAATGGTGAGTGGA | 8355 | TCCACTCACCATTCCTGCCTGCC |
| 7092 | GGGCAGGCAGGAATGGTGAGTGG | 8356 | CCACTCACCATTCCTGCCTGCCC |
| 7093 | TGGGCAGGCAGGAATGGTGAGTG | 8357 | CACTCACCATTCCTGCCTGCCCA |
| 7094 | CTGGGCAGGCAGGAATGGTGAGT | 8358 | ACTCACCATTCCTGCCTGCCCAG |
| 7095 | GCTGGGCAGGCAGGAATGGTGAG | 8359 | CTCACCATTCCTGCCTGCCCAGC |
| 7096 | AGCTGGGCAGGCAGGAATGGTGA | 8360 | TCACCATTCCTGCCTGCCCAGCT |
| 7097 | GAGCTGGGCAGGCAGGAATGGTG | 8361 | CACCATTCCTGCCTGCCCAGCTC |
| 7098 | AGAGCTGGGCAGGCAGGAATGGT | 8362 | ACCATTCCTGCCTGCCCAGCTCT |
| 7099 | AAGAGCTGGGCAGGCAGGAATGG | 8363 | CCATTCCTGCCTGCCCAGCTCTT |
| 7100 | GAAGAGCTGGGCAGGCAGGAATG | 8364 | CATTCCTGCCTGCCCAGCTCTTC |
| 7101 | GGAAGAGCTGGGCAGGCAGGAAT | 8365 | ATTCCTGCCTGCCCAGCTCTTCC |
| 7102 | AGGAAGAGCTGGGCAGGCAGGAA | 8366 | TTCCTGCCTGCCCAGCTCTTCCT |
| 7103 | GAGGAAGAGCTGGGCAGGCAGGA | 8367 | TCCTGCCTGCCCAGCTCTTCCTC |
| 7104 | AGAGGAAGAGCTGGGCAGGCAGG | 8368 | CCTGCCTGCCCAGCTCTTCCTCT |
| 7105 | AAGAGGAAGAGCTGGGCAGGCAG | 8369 | CTGCCTGCCCAGCTCTTCCTCTT |
| 7106 | AAAGAGGAAGAGCTGGGCAGGCA | 8370 | TGCCTGCCCAGCTCTTCCTCTTT |
| 7107 | GAAAGAGGAAGAGCTGGGCAGGC | 8371 | GCCTGCCCAGCTCTTCCTCTTTC |
| 7108 | AGAAAGAGGAAGAGCTGGGCAGG | 8372 | CCTGCCCAGCTCTTCCTCTTTCT |
| 7109 | CAGAAAGAGGAAGAGCTGGGCAG | 8373 | CTGCCCAGCTCTTCCTCTTTCTG |
| 7110 | CCAGAAAGAGGAAGAGCTGGGCA | 8374 | TGCCCAGCTCTTCCTCTTTCTGG |
| 7111 | GCCAGAAAGAGGAAGAGCTGGGC | 8375 | GCCCAGCTCTTCCTCTTTCTGGC |
| 7112 | GGCCAGAAAGAGGAAGAGCTGGG | 8376 | CCCAGCTCTTCCTCTTTCTGGCC |
| 7113 | TGGCCAGAAAGAGGAAGAGCTGG | 8377 | CCAGCTCTTCCTCTTTCTGGCCA |
| 7114 | GTGGCCAGAAAGAGGAAGAGCTG | 8378 | CAGCTCTTCCTCTTTCTGGCCAC |
| 7115 | TGTGGCCAGAAAGAGGAAGAGCT | 8379 | AGCTCTTCCTCTTTCTGGCCACA |
| 7116 | GTGTGGCCAGAAAGAGGAAGAGC | 8380 | GCTCTTCCTCTTTCTGGCCACAC |
| 7117 | GGTGTGGCCAGAAAGAGGAAGAG | 8381 | CTCTTCCTCTTTCTGGCCACACC |
| 7118 | TGGTGTGGCCAGAAAGAGGAAGA | 8382 | TCTTCCTCTTTCTGGCCACACCA |
| 7119 | ATGGTGTGGCCAGAAAGAGGAAG | 8383 | CTTCCTCTTTCTGGCCACACCAT |
| 7120 | GATGGTGTGGCCAGAAAGAGGAA | 8384 | TTCCTCTTTCTGGCCACACCATC |
| 7121 | GGATGGTGTGGCCAGAAAGAGGA | 8385 | TCCTCTTTCTGGCCACACCATCC |
| 7122 | TGGATGGTGTGGCCAGAAAGAGG | 8386 | CCTCTTTCTGGCCACACCATCCA |
| 7123 | GTGGATGGTGTGGCCAGAAAGAG | 8387 | CTCTTTCTGGCCACACCATCCAC |
| 7124 | TGTGGATGGTGTGGCCAGAAAGA | 8388 | TCTTTCTGGCCACACCATCCACA |
| 7125 | GTGTGGATGGTGTGGCCAGAAAG | 8389 | CTTTCTGGCCACACCATCCACAC |
| 7126 | AGTGTGGATGGTGTGGCCAGAAA | 8390 | TTTCTGGCCACACCATCCACACT |
| 7127 | GAGTGTGGATGGTGTGGCCAGAA | 8391 | TTCTGGCCACACCATCCACACTC |
| 7128 | AGAGTGTGGATGGTGTGGCCAGA | 8392 | TCTGGCCACACCATCCACACTCT |
| 7129 | GAGAGTGTGGATGGTGTGGCCAG | 8393 | CTGGCCACACCATCCACACTCTC |
| 7130 | GGAGAGTGTGGATGGTGTGGCCA | 8394 | TGGCCACACCATCCACACTCTCC |
| 7131 | AGGAGAGTGTGGATGGTGTGGCC | 8395 | GGCCACACCATCCACACTCTCCT |
| 7132 | CAGGAGAGTGTGGATGGTGTGGC | 8396 | GCCACACCATCCACACTCTCCTG |
| 7133 | CCAGGAGAGTGTGGATGGTGTGG | 8397 | CCACACCATCCACACTCTCCTGG |
| 7134 | GCCAGGAGAGTGTGGATGGTGTG | 8398 | CACACCATCCACACTCTCCTGGC |
| 7135 | GGCCAGGAGAGTGTGGATGGTGT | 8399 | ACACCATCCACACTCTCCTGGCC |
| 7136 | GGGCCAGGAGAGTGTGGATGGTG | 8400 | CACCATCCACACTCTCCTGGCCC |
| 7137 | AGGGCCAGGAGAGTGTGGATGGT | 8401 | ACCATCCACACTCTCCTGGCCCT |
| 7138 | GAGGGCCAGGAGAGTGTGGATGG | 8402 | CCATCCACACTCTCCTGGCCCTC |
| 7139 | AGAGGGCCAGGAGAGTGTGGATG | 8403 | CATCCACACTCTCCTGGCCCTCT |
| 7140 | CAGAGGGCCAGGAGAGTGTGGAT | 8404 | ATCCACACTCTCCTGGCCCTCTG |
| 7141 | TCAGAGGGCCAGGAGAGTGTGGA | 8405 | TCCACACTCTCCTGGCCCTCTGA |
| 7142 | CTCAGAGGGCCAGGAGAGTGTGG | 8406 | CCACACTCTCCTGGCCCTCTGAG |
| 7143 | TCTCAGAGGGCCAGGAGAGTGTG | 8407 | CACACTCTCCTGGCCCTCTGAGA |
| 7144 | GTCTCAGAGGGCCAGGAGAGTGT | 8408 | ACACTCTCCTGGCCCTCTGAGAC |
| 7145 | AGTCTCAGAGGGCCAGGAGAGTG | 8409 | CACTCTCCTGGCCCTCTGAGACT |
| 7146 | CAGTCTCAGAGGGCCAGGAGAGT | 8410 | ACTCTCCTGGCCCTCTGAGACTG |
| 7147 | GCAGTCTCAGAGGGCCAGGAGAG | 8411 | CTCTCCTGGCCCTCTGAGACTGC |
| 7148 | GGCAGTCTCAGAGGGCCAGGAGA | 8412 | TCTCCTGGCCCTCTGAGACTGCC |
| 7149 | GGGCAGTCTCAGAGGGCCAGGAG | 8413 | CTCCTGGCCCTCTGAGACTGCCC |
| 7150 | CGGGCAGTCTCAGAGGGCCAGGA | 8414 | TCCTGGCCCTCTGAGACTGCCCG |
| 7151 | GCGGGCAGTCTCAGAGGGCCAGG | 8415 | CCTGGCCCTCTGAGACTGCCCGC |
| 7152 | GGCGGGCAGTCTCAGAGGGCCAG | 8416 | CTGGCCCTCTGAGACTGCCCGCC |
| 7153 | TGGCGGGCAGTCTCAGAGGGCCA | 8417 | TGGCCCTCTGAGACTGCCCGCCA |
| 7154 | ATGGCGGGCAGTCTCAGAGGGCC | 8418 | GGCCCTCTGAGACTGCCCGCCAT |
| 7155 | CATGGCGGGCAGTCTCAGAGGGC | 8419 | GCCCTCTGAGACTGCCCGCCATG |
| 7156 | GCATGGCGGGCAGTCTCAGAGGG | 8420 | CCCTCTGAGACTGCCCGCCATGC |
| 7157 | GGCATGGCGGGCAGTCTCAGAGG | 8421 | CCTCTGAGACTGCCCGCCATGCC |
| 7158 | TGGCATGGCGGGCAGTCTCAGAG | 8422 | CTCTGAGACTGCCCGCCATGCCA |
| 7159 | ATGGCATGGCGGGCAGTCTCAGA | 8423 | TCTGAGACTGCCCGCCATGCCAT |
| 7160 | AATGGCATGGCGGGCAGTCTCAG | 8424 | CTGAGACTGCCCGCCATGCCATT |
| 7161 | GAATGGCATGGCGGGCAGTCTCA | 8425 | TGAGACTGCCCGCCATGCCATTC |
| 7162 | GGAATGGCATGGCGGGCAGTCTC | 8426 | GAGACTGCCCGCCATGCCATTCC |
| 7163 | GGGAATGGCATGGCGGGCAGTCT | 8427 | AGACTGCCCGCCATGCCATTCCC |
| 7164 | AGGGAATGGCATGGCGGGCAGTC | 8428 | GACTGCCCGCCATGCCATTCCCT |
| 7165 | AAGGGAATGGCATGGCGGGCAGT | 8429 | ACTGCCCGCCATGCCATTCCCTT |
| 7166 | AAAGGGAATGGCATGGCGGGCAG | 8430 | CTGCCCGCCATGCCATTCCCTTT |
| 7167 | TAAAGGGAATGGCATGGCGGGCA | 8431 | TGCCCGCCATGCCATTCCCTTTA |
| 7168 | GTAAAGGGAATGGCATGGCGGGC | 8432 | GCCCGCCATGCCATTCCCTTTAC |
| 7169 | GGTAAAGGGAATGGCATGGCGGG | 8433 | CCCGCCATGCCATTCCCTTTACC |
| 7170 | AGGTAAAGGGAATGGCATGGCGG | 8434 | CCGCCATGCCATTCCCTTTACCT |
| 7171 | CAGGTAAAGGGAATGGCATGGCG | 8435 | CGCCATGCCATTCCCTTTACCTG |
| 7172 | CCAGGTAAAGGGAATGGCATGGC | 8436 | GCCATGCCATTCCCTTTACCTGG |
| 7173 | TCCAGGTAAAGGGAATGGCATGG | 8437 | CCATGCCATTCCCTTTACCTGGA |
| 7174 | TTCCAGGTAAAGGGAATGGCATG | 8438 | CATGCCATTCCCTTTACCTGGAA |
| 7175 | TTTCCAGGTAAAGGGAATGGCAT | 8439 | ATGCCATTCCCTTTACCTGGAAA |
| 7176 | TTTTCCAGGTAAAGGGAATGGCA | 8440 | TGCCATTCCCTTTACCTGGAAAA |
| 7177 | GTTTTCCAGGTAAAGGGAATGGC | 8441 | GCCATTCCCTTTACCTGGAAAAC |
| 7178 | AGTTTTCCAGGTAAAGGGAATGG | 8442 | CCATTCCCTTTACCTGGAAAACT |
| 7179 | GAGTTTTCCAGGTAAAGGGAATG | 8443 | CATTCCCTTTACCTGGAAAACTC |
| 7180 | GGAGTTTTCCAGGTAAAGGGAAT | 8444 | ATTCCCTTTACCTGGAAAACTCC |
| 7181 | AGGAGTTTTCCAGGTAAAGGGAA | 8445 | TTCCCTTTACCTGGAAAACTCCT |
| 7182 | GAGGAGTTTTCCAGGTAAAGGGA | 8446 | TCCCTTTACCTGGAAAACTCCTC |
| 7183 | GGAGGAGTTTTCCAGGTAAAGGG | 8447 | CCCTTTACCTGGAAAACTCCTCC |
| 7184 | GGGAGGAGTTTTCCAGGTAAAGG | 8448 | CCTTTACCTGGAAAACTCCTCCC |
| 7185 | AGGGAGGAGTTTTCCAGGTAAAG | 8449 | CTTTACCTGGAAAACTCCTCCCT |
| 7186 | TAGGGAGGAGTTTTCCAGGTAAA | 8450 | TTTACCTGGAAAACTCCTCCCTA |
| 7187 | ATAGGGAGGAGTTTTCCAGGTAA | 8451 | TTACCTGGAAAACTCCTCCCTAT |
| 7188 | GATAGGGAGGAGTTTTCCAGGTA | 8452 | TACCTGGAAAACTCCTCCCTATC |
| 7189 | GGATAGGGAGGAGTTTTCCAGGT | 8453 | ACCTGGAAAACTCCTCCCTATCC |
| 7190 | TGGATAGGGAGGAGTTTTCCAGG | 8454 | CCTGGAAAACTCCTCCCTATCCA |
| 7191 | ATGGATAGGGAGGAGTTTTCCAG | 8455 | CTGGAAAACTCCTCCCTATCCAT |
| 7192 | GATGGATAGGGAGGAGTTTTCCA | 8456 | TGGAAAACTCCTCCCTATCCATC |
| 7193 | TGATGGATAGGGAGGAGTTTTCC | 8457 | GGAAAACTCCTCCCTATCCATCA |
| 7194 | TTGATGGATAGGGAGGAGTTTTC | 8458 | GAAAACTCCTCCCTATCCATCAA |
| 7195 | TTTGATGGATAGGGAGGAGTTTT | 8459 | AAAACTCCTCCCTATCCATCAAA |
| 7196 | CTTTGATGGATAGGGAGGAGTTT | 8460 | AAACTCCTCCCTATCCATCAAAG |
| 7197 | ACTTTGATGGATAGGGAGGAGTT | 8461 | AACTCCTCCCTATCCATCAAAGT |
| 7198 | GACTTTGATGGATAGGGAGGAGT | 8462 | ACTCCTCCCTATCCATCAAAGTC |
| 7199 | GGACTTTGATGGATAGGGAGGAG | 8463 | CTCCTCCCTATCCATCAAAGTCC |
| 7200 | TGGACTTTGATGGATAGGGAGGA | 8464 | TCCTCCCTATCCATCAAAGTCCA |
| 7201 | CTGGACTTTGATGGATAGGGAGG | 8465 | CCTCCCTATCCATCAAAGTCCAG |
| 7202 | TCTGGACTTTGATGGATAGGGAG | 8466 | CTCCCTATCCATCAAAGTCCAGA |
| 7203 | ATCTGGACTTTGATGGATAGGGA | 8467 | TCCCTATCCATCAAAGTCCAGAT |
| 7204 | AATCTGGACTTTGATGGATAGGG | 8468 | CCCTATCCATCAAAGTCCAGATT |
| 7205 | GAATCTGGACTTTGATGGATAGG | 8469 | CCTATCCATCAAAGTCCAGATTC |
| 7206 | TGAATCTGGACTTTGATGGATAG | 8470 | CTATCCATCAAAGTCCAGATTCA |
| 7207 | CTGAATCTGGACTTTGATGGATA | 8471 | TATCCATCAAAGTCCAGATTCAG |
| 7208 | CCTGAATCTGGACTTTGATGGAT | 8472 | ATCCATCAAAGTCCAGATTCAGG |
| 7209 | CCCTGAATCTGGACTTTGATGGA | 8473 | TCCATCAAAGTCCAGATTCAGGG |
| 7210 | ACCCTGAATCTGGACTTTGATGG | 8474 | CCATCAAAGTCCAGATTCAGGGT |
| 7211 | GACCCTGAATCTGGACTTTGATG | 8475 | CATCAAAGTCCAGATTCAGGGTC |
| 7212 | TGACCCTGAATCTGGACTTTGAT | 8476 | ATCAAAGTCCAGATTCAGGGTCA |
| 7213 | GTGACCCTGAATCTGGACTTTGA | 8477 | TCAAAGTCCAGATTCAGGGTCAC |
| 7214 | GGTGACCCTGAATCTGGACTTTG | 8478 | CAAAGTCCAGATTCAGGGTCACC |
| 7215 | AGGTGACCCTGAATCTGGACTTT | 8479 | AAAGTCCAGATTCAGGGTCACCT |
| 7216 | GAGGTGACCCTGAATCTGGACTT | 8480 | AAGTCCAGATTCAGGGTCACCTC |
| 7217 | GGAGGTGACCCTGAATCTGGACT | 8481 | AGTCCAGATTCAGGGTCACCTCC |
| 7218 | AGGAGGTGACCCTGAATCTGGAC | 8482 | GTCCAGATTCAGGGTCACCTCCT |
| 7219 | GAGGAGGTGACCCTGAATCTGGA | 8483 | TCCAGATTCAGGGTCACCTCCTC |
| 7220 | AGAGGAGGTGACCCTGAATCTGG | 8484 | CCAGATTCAGGGTCACCTCCTCT |
| 7221 | CAGAGGAGGTGACCCTGAATCTG | 8485 | CAGATTCAGGGTCACCTCCTCTG |
| 7222 | CCAGAGGAGGTGACCCTGAATCT | 8486 | AGATTCAGGGTCACCTCCTCTGG |
| 7223 | CCCAGAGGAGGTGACCCTGAATC | 8487 | GATTCAGGGTCACCTCCTCTGGG |
| 7224 | TCCCAGAGGAGGTGACCCTGAAT | 8488 | ATTCAGGGTCACCTCCTCTGGGA |
| 7225 | TTCCCAGAGGAGGTGACCCTGAA | 8489 | TTCAGGGTCACCTCCTCTGGGAA |
| 7226 | CTTCCCAGAGGAGGTGACCCTGA | 8490 | TCAGGGTCACCTCCTCTGGGAAG |
| 7227 | GCTTCCCAGAGGAGGTGACCCTG | 8491 | CAGGGTCACCTCCTCTGGGAAGC |
| 7228 | GGCTTCCCAGAGGAGGTGACCCT | 8492 | AGGGTCACCTCCTCTGGGAAGCC |
| 7229 | GGGCTTCCCAGAGGAGGTGACCC | 8493 | GGGTCACCTCCTCTGGGAAGCCC |
| 7230 | TGGGCTTCCCAGAGGAGGTGACC | 8494 | GGTCACCTCCTCTGGGAAGCCCA |
| 7231 | GTGGGCTTCCCAGAGGAGGTGAC | 8495 | GTCACCTCCTCTGGGAAGCCCAC |
| 7232 | GGTGGGCTTCCCAGAGGAGGTGA | 8496 | TCACCTCCTCTGGGAAGCCCACC |
| 7233 | AGGTGGGCTTCCCAGAGGAGGTG | 8497 | CACCTCCTCTGGGAAGCCCACCT |
| 7234 | AAGGTGGGCTTCCCAGAGGAGGT | 8498 | ACCTCCTCTGGGAAGCCCACCTT |
| 7235 | CAAGGTGGGCTTCCCAGAGGAGG | 8499 | CCTCCTCTGGGAAGCCCACCTTG |
| 7236 | CCAAGGTGGGCTTCCCAGAGGAG | 8500 | CTCCTCTGGGAAGCCCACCTTGG |
| 7237 | GCCAAGGTGGGCTTCCCAGAGGA | 8501 | TCCTCTGGGAAGCCCACCTTGGC |
| 7238 | GGCCAAGGTGGGCTTCCCAGAGG | 8502 | CCTCTGGGAAGCCCACCTTGGCC |
| 7239 | AGGCCAAGGTGGGCTTCCCAGAG | 8503 | CTCTGGGAAGCCCACCTTGGCCT |
| 7240 | GAGGCCAAGGTGGGCTTCCCAGA | 8504 | TCTGGGAAGCCCACCTTGGCCTC |
| 7241 | GGAGGCCAAGGTGGGCTTCCCAG | 8505 | CTGGGAAGCCCACCTTGGCCTCC |
| 7242 | TGGAGGCCAAGGTGGGCTTCCCA | 8506 | TGGGAAGCCCACCTTGGCCTCCA |
| 7243 | CTGGAGGCCAAGGTGGGCTTCCC | 8507 | GGGAAGCCCACCTTGGCCTCCAG |
| 7244 | CCTGGAGGCCAAGGTGGGCTTCC | 8508 | GGAAGCCCACCTTGGCCTCCAGG |
| 7245 | ACCTGGAGGCCAAGGTGGGCTTC | 8509 | GAAGCCCACCTTGGCCTCCAGGT |
| 7246 | AACCTGGAGGCCAAGGTGGGCTT | 8510 | AAGCCCACCTTGGCCTCCAGGTT |
| 7247 | CAACCTGGAGGCCAAGGTGGGCT | 8511 | AGCCCACCTTGGCCTCCAGGTTG |
| 7248 | TCAACCTGGAGGCCAAGGTGGGC | 8512 | GCCCACCTTGGCCTCCAGGTTGA |
| 7249 | GTCAACCTGGAGGCCAAGGTGGG | 8513 | CCCACCTTGGCCTCCAGGTTGAC |
| 7250 | AGTCAACCTGGAGGCCAAGGTGG | 8514 | CCACCTTGGCCTCCAGGTTGACT |
| 7251 | GAGTCAACCTGGAGGCCAAGGTG | 8515 | CACCTTGGCCTCCAGGTTGACTC |
| 7252 | AGAGTCAACCTGGAGGCCAAGGT | 8516 | ACCTTGGCCTCCAGGTTGACTCT |
| 7253 | GAGAGTCAACCTGGAGGCCAAGG | 8517 | CCTTGGCCTCCAGGTTGACTCTC |
| 7254 | TGAGAGTCAACCTGGAGGCCAAG | 8518 | CTTGGCCTCCAGGTTGACTCTCA |
| 7255 | GTGAGAGTCAACCTGGAGGCCAA | 8519 | TTGGCCTCCAGGTTGACTCTCAC |
| 7256 | AGTGAGAGTCAACCTGGAGGCCA | 8520 | TGGCCTCCAGGTTGACTCTCACT |
| 7257 | TAGTGAGAGTCAACCTGGAGGCC | 8521 | GGCCTCCAGGTTGACTCTCACTA |
| 7258 | GTAGTGAGAGTCAACCTGGAGGC | 8522 | GCCTCCAGGTTGACTCTCACTAC |
| 7259 | AGTAGTGAGAGTCAACCTGGAGG | 8523 | CCTCCAGGTTGACTCTCACTACT |
| 7260 | GAGTAGTGAGAGTCAACCTGGAG | 8524 | CTCCAGGTTGACTCTCACTACTC |
| 7261 | TGAGTAGTGAGAGTCAACCTGGA | 8525 | TCCAGGTTGACTCTCACTACTCA |
| 7262 | ATGAGTAGTGAGAGTCAACCTGG | 8526 | CCAGGTTGACTCTCACTACTCAT |
| 7263 | GATGAGTAGTGAGAGTCAACCTG | 8527 | CAGGTTGACTCTCACTACTCATC |
| 7264 | TGATGAGTAGTGAGAGTCAACCT | 8528 | AGGTTGACTCTCACTACTCATCA |
| 7265 | ATGATGAGTAGTGAGAGTCAACC | 8529 | GGTTGACTCTCACTACTCATCAT |
| 7266 | GATGATGAGTAGTGAGAGTCAAC | 8530 | GTTGACTCTCACTACTCATCATC |
| 7267 | TGATGATGAGTAGTGAGAGTCAA | 8531 | TTGACTCTCACTACTCATCATCA |
| 7268 | CTGATGATGAGTAGTGAGAGTCA | 8532 | TGACTCTCACTACTCATCATCAG |
| 7269 | CCTGATGATGAGTAGTGAGAGTC | 8533 | GACTCTCACTACTCATCATCAGG |
| 7270 | ACCTGATGATGAGTAGTGAGAGT | 8534 | ACTCTCACTACTCATCATCAGGT |
| 7271 | AACCTGATGATGAGTAGTGAGAG | 8535 | CTCTCACTACTCATCATCAGGTT |
| 7272 | GAACCTGATGATGAGTAGTGAGA | 8536 | TCTCACTACTCATCATCAGGTTC |
| 7273 | AGAACCTGATGATGAGTAGTGAG | 8537 | CTCACTACTCATCATCAGGTTCT |
| 7274 | AAGAACCTGATGATGAGTAGTGA | 8538 | TCACTACTCATCATCAGGTTCTT |
| 7275 | GAAGAACCTGATGATGAGTAGTG | 8539 | CACTACTCATCATCAGGTTCTTC |
| 7276 | GGAAGAACCTGATGATGAGTAGT | 8540 | ACTACTCATCATCAGGTTCTTCC |
| 7277 | AGGAAGAACCTGATGATGAGTAG | 8541 | CTACTCATCATCAGGTTCTTCCT |
| 7278 | AAGGAAGAACCTGATGATGAGTA | 8542 | TACTCATCATCAGGTTCTTCCTT |
| 7279 | GAAGGAAGAACCTGATGATGAGT | 8543 | ACTCATCATCAGGTTCTTCCTTC |
| 7280 | AGAAGGAAGAACCTGATGATGAG | 8544 | CTCATCATCAGGTTCTTCCTTCT |
| 7281 | TAGAAGGAAGAACCTGATGATGA | 8545 | TCATCATCAGGTTCTTCCTTCTA |
| 7282 | ATAGAAGGAAGAACCTGATGATG | 8546 | CATCATCAGGTTCTTCCTTCTAT |
| 7283 | AATAGAAGGAAGAACCTGATGAT | 8547 | ATCATCAGGTTCTTCCTTCTATT |
| 7284 | GAATAGAAGGAAGAACCTGATGA | 8548 | TCATCAGGTTCTTCCTTCTATTC |
| 7285 | GGAATAGAAGGAAGAACCTGATG | 8549 | CATCAGGTTCTTCCTTCTATTCC |
| 7286 | TGGAATAGAAGGAAGAACCTGAT | 8550 | ATCAGGTTCTTCCTTCTATTCCA |
| 7287 | CTGGAATAGAAGGAAGAACCTGA | 8551 | TCAGGTTCTTCCTTCTATTCCAG |
| 7288 | GCTGGAATAGAAGGAAGAACCTG | 8552 | CAGGTTCTTCCTTCTATTCCAGC |
| 7289 | GGCTGGAATAGAAGGAAGAACCT | 8553 | AGGTTCTTCCTTCTATTCCAGCC |
| 7290 | GGGCTGGAATAGAAGGAAGAACC | 8554 | GGTTCTTCCTTCTATTCCAGCCC |
| 7291 | AGGGCTGGAATAGAAGGAAGAAC | 8555 | GTTCTTCCTTCTATTCCAGCCCT |
| 7292 | TAGGGCTGGAATAGAAGGAAGAA | 8556 | TTCTTCCTTCTATTCCAGCCCTA |
| 7293 | TTAGGGCTGGAATAGAAGGAAGA | 8557 | TCTTCCTTCTATTCCAGCCCTAA |
| 7294 | GTTAGGGCTGGAATAGAAGGAAG | 8558 | CTTCCTTCTATTCCAGCCCTAAC |
| 7295 | GGTTAGGGCTGGAATAGAAGGAA | 8559 | TTCCTTCTATTCCAGCCCTAACC |
| 7296 | TGGTTAGGGCTGGAATAGAAGGA | 8560 | TCCTTCTATTCCAGCCCTAACCA |
| 7297 | GTGGTTAGGGCTGGAATAGAAGG | 8561 | CCTTCTATTCCAGCCCTAACCAC |
| 7298 | AGTGGTTAGGGCTGGAATAGAAG | 8562 | CTTCTATTCCAGCCCTAACCACT |
| 7299 | GAGTGGTTAGGGCTGGAATAGAA | 8563 | TTCTATTCCAGCCCTAACCACTC |
| 7300 | TGAGTGGTTAGGGCTGGAATAGA | 8564 | TCTATTCCAGCCCTAACCACTCA |
| 7301 | CTGAGTGGTTAGGGCTGGAATAG | 8565 | CTATTCCAGCCCTAACCACTCAG |
| 7302 | CCTGAGTGGTTAGGGCTGGAATA | 8566 | TATTCCAGCCCTAACCACTCAGG |
| 7303 | TCCTGAGTGGTTAGGGCTGGAAT | 8567 | ATTCCAGCCCTAACCACTCAGGA |
| 7304 | ATCCTGAGTGGTTAGGGCTGGAA | 8568 | TTCCAGCCCTAACCACTCAGGAT |
| 7305 | AATCCTGAGTGGTTAGGGCTGGA | 8569 | TCCAGCCCTAACCACTCAGGATT |
| 7306 | CAATCCTGAGTGGTTAGGGCTGG | 8570 | CCAGCCCTAACCACTCAGGATTG |
| 7307 | CCAATCCTGAGTGGTTAGGGCTG | 8571 | CAGCCCTAACCACTCAGGATTGG |
| 7308 | CCCAATCCTGAGTGGTTAGGGCT | 8572 | AGCCCTAACCACTCAGGATTGGG |
| 7309 | GCCCAATCCTGAGTGGTTAGGGC | 8573 | GCCCTAACCACTCAGGATTGGGC |
| 7310 | GGCCCAATCCTGAGTGGTTAGGG | 8574 | CCCTAACCACTCAGGATTGGGCC |
| 7311 | CGGCCCAATCCTGAGTGGTTAGG | 8575 | CCTAACCACTCAGGATTGGGCCG |
| 7312 | ACGGCCCAATCCTGAGTGGTTAG | 8576 | CTAACCACTCAGGATTGGGCCGT |
| 7313 | AACGGCCCAATCCTGAGTGGTTA | 8577 | TAACCACTCAGGATTGGGCCGTT |
| 7314 | AAACGGCCCAATCCTGAGTGGTT | 8578 | AACCACTCAGGATTGGGCCGTTT |
| 7315 | CAAACGGCCCAATCCTGAGTGGT | 8579 | ACCACTCAGGATTGGGCCGTTTG |
| 7316 | ACAAACGGCCCAATCCTGAGTGG | 8580 | CCACTCAGGATTGGGCCGTTTGT |
| 7317 | CACAAACGGCCCAATCCTGAGTG | 8581 | CACTCAGGATTGGGCCGTTTGTG |
| 7318 | ACACAAACGGCCCAATCCTGAGT | 8582 | ACTCAGGATTGGGCCGTTTGTGT |
| 7319 | GACACAAACGGCCCAATCCTGAG | 8583 | CTCAGGATTGGGCCGTTTGTGTC |
| 7320 | AGACACAAACGGCCCAATCCTGA | 8584 | TCAGGATTGGGCCGTTTGTGTCT |
| 7321 | CAGACACAAACGGCCCAATCCTG | 8585 | CAGGATTGGGCCGTTTGTGTCTG |
| 7322 | CCAGACACAAACGGCCCAATCCT | 8586 | AGGATTGGGCCGTTTGTGTCTGG |
| 7323 | CCCAGACACAAACGGCCCAATCC | 8587 | GGATTGGGCCGTTTGTGTCTGGG |
| 7324 | ACCCAGACACAAACGGCCCAATC | 8588 | GATTGGGCCGTTTGTGTCTGGGT |
| 7325 | TACCCAGACACAAACGGCCCAAT | 8589 | ATTGGGCCGTTTGTGTCTGGGTA |
| 7326 | ATACCCAGACACAAACGGCCCAA | 8590 | TTGGGCCGTTTGTGTCTGGGTAT |
| 7327 | CATACCCAGACACAAACGGCCCA | 8591 | TGGGCCGTTTGTGTCTGGGTATG |
| 7328 | ACATACCCAGACACAAACGGCCC | 8592 | GGGCCGTTTGTGTCTGGGTATGT |
| 7329 | GACATACCCAGACACAAACGGCC | 8593 | GGCCGTTTGTGTCTGGGTATGTC |
| 7330 | AGACATACCCAGACACAAACGGC | 8594 | GCCGTTTGTGTCTGGGTATGTCT |
| 7331 | GAGACATACCCAGACACAAACGG | 8595 | CCGTTTGTGTCTGGGTATGTCTC |
| 7332 | AGAGACATACCCAGACACAAACG | 8596 | CGTTTGTGTCTGGGTATGTCTCT |
| 7333 | AAGAGACATACCCAGACACAAAC | 8597 | GTTTGTGTCTGGGTATGTCTCTT |
| 7334 | GAAGAGACATACCCAGACACAAA | 8598 | TTTGTGTCTGGGTATGTCTCTTC |
| 7335 | GGAAGAGACATACCCAGACACAA | 8599 | TTGTGTCTGGGTATGTCTCTTCC |
| 7336 | TGGAAGAGACATACCCAGACACA | 8600 | TGTGTCTGGGTATGTCTCTTCCA |
| 7337 | CTGGAAGAGACATACCCAGACAC | 8601 | GTGTCTGGGTATGTCTCTTCCAG |
| 7338 | GCTGGAAGAGACATACCCAGACA | 8602 | TGTCTGGGTATGTCTCTTCCAGC |
| 7339 | AGCTGGAAGAGACATACCCAGAC | 8603 | GTCTGGGTATGTCTCTTCCAGCT |
| 7340 | CAGCTGGAAGAGACATACCCAGA | 8604 | TCTGGGTATGTCTCTTCCAGCTG |
| 7341 | GCAGCTGGAAGAGACATACCCAG | 8605 | CTGGGTATGTCTCTTCCAGCTGC |
| 7342 | GGCAGCTGGAAGAGACATACCCA | 8606 | TGGGTATGTCTCTTCCAGCTGCC |
| 7343 | AGGCAGCTGGAAGAGACATACCC | 8607 | GGGTATGTCTCTTCCAGCTGCCT |
| 7344 | CAGGCAGCTGGAAGAGACATACC | 8608 | GGTATGTCTCTTCCAGCTGCCTG |
| 7345 | CCAGGCAGCTGGAAGAGACATAC | 8609 | GTATGTCTCTTCCAGCTGCCTGG |
| 7346 | CCCAGGCAGCTGGAAGAGACATA | 8610 | TATGTCTCTTCCAGCTGCCTGGG |
| 7347 | ACCCAGGCAGCTGGAAGAGACAT | 8611 | ATGTCTCTTCCAGCTGCCTGGGT |
| 7348 | AACCCAGGCAGCTGGAAGAGACA | 8612 | TGTCTCTTCCAGCTGCCTGGGTT |
| 7349 | AAACCCAGGCAGCTGGAAGAGAC | 8613 | GTCTCTTCCAGCTGCCTGGGTTT |
| 7350 | GAAACCCAGGCAGCTGGAAGAGA | 8614 | TCTCTTCCAGCTGCCTGGGTTTC |
| 7351 | GGAAACCCAGGCAGCTGGAAGAG | 8615 | CTCTTCCAGCTGCCTGGGTTTCC |
| 7352 | AGGAAACCCAGGCAGCTGGAAGA | 8616 | TCTTCCAGCTGCCTGGGTTTCCT |
| 7353 | CAGGAAACCCAGGCAGCTGGAAG | 8617 | CTTCCAGCTGCCTGGGTTTCCTG |
| 7354 | CCAGGAAACCCAGGCAGCTGGAA | 8618 | TTCCAGCTGCCTGGGTTTCCTGG |
| 7355 | TCCAGGAAACCCAGGCAGCTGGA | 8619 | TCCAGCTGCCTGGGTTTCCTGGA |
| 7356 | TTCCAGGAAACCCAGGCAGCTGG | 8620 | CCAGCTGCCTGGGTTTCCTGGAA |
| 7357 | TTTCCAGGAAACCCAGGCAGCTG | 8621 | CAGCTGCCTGGGTTTCCTGGAAA |
| 7358 | CTTTCCAGGAAACCCAGGCAGCT | 8622 | AGCTGCCTGGGTTTCCTGGAAAG |
| 7359 | TCTTTCCAGGAAACCCAGGCAGC | 8623 | GCTGCCTGGGTTTCCTGGAAAGA |
| 7360 | TTCTTTCCAGGAAACCCAGGCAG | 8624 | CTGCCTGGGTTTCCTGGAAAGAA |
| 7361 | GTTCTTTCCAGGAAACCCAGGCA | 8625 | TGCCTGGGTTTCCTGGAAAGAAC |
| 7362 | AGTTCTTTCCAGGAAACCCAGGC | 8626 | GCCTGGGTTTCCTGGAAAGAACT |
| 7363 | GAGTTCTTTCCAGGAAACCCAGG | 8627 | CCTGGGTTTCCTGGAAAGAACTC |
| 7364 | AGAGTTCTTTCCAGGAAACCCAG | 8628 | CTGGGTTTCCTGGAAAGAACTCT |
| 7365 | AAGAGTTCTTTCCAGGAAACCCA | 8629 | TGGGTTTCCTGGAAAGAACTCTT |
| 7366 | TAAGAGTTCTTTCCAGGAAACCC | 8630 | GGGTTTCCTGGAAAGAACTCTTA |
| 7367 | ATAAGAGTTCTTTCCAGGAAACC | 8631 | GGTTTCCTGGAAAGAACTCTTAT |
| 7368 | GATAAGAGTTCTTTCCAGGAAAC | 8632 | GTTTCCTGGAAAGAACTCTTATC |
| 7369 | GGATAAGAGTTCTTTCCAGGAAA | 8633 | TTTCCTGGAAAGAACTCTTATCC |
| 7370 | GGGATAAGAGTTCTTTCCAGGAA | 8634 | TTCCTGGAAAGAACTCTTATCCC |
| 7371 | GGGGATAAGAGTTCTTTCCAGGA | 8635 | TCCTGGAAAGAACTCTTATCCCC |
| 7372 | TGGGGATAAGAGTTCTTTCCAGG | 8636 | CCTGGAAAGAACTCTTATCCCCA |
| 7373 | CTGGGGATAAGAGTTCTTTCCAG | 8637 | CTGGAAAGAACTCTTATCCCCAG |
| 7374 | CCTGGGGATAAGAGTTCTTTCCA | 8638 | TGGAAAGAACTCTTATCCCCAGG |
| 7375 | TCCTGGGGATAAGAGTTCTTTCC | 8639 | GGAAAGAACTCTTATCCCCAGGA |
| 7376 | TTCCTGGGGATAAGAGTTCTTTC | 8640 | GAAAGAACTCTTATCCCCAGGAA |
| 7377 | GTTCCTGGGGATAAGAGTTCTTT | 8641 | AAAGAACTCTTATCCCCAGGAAC |
| 7378 | AGTTCCTGGGGATAAGAGTTCTT | 8642 | AAGAACTCTTATCCCCAGGAACT |
| 7379 | TAGTTCCTGGGGATAAGAGTTCT | 8643 | AGAACTCTTATCCCCAGGAACTA |
| 7380 | CTAGTTCCTGGGGATAAGAGTTC | 8644 | GAACTCTTATCCCCAGGAACTAG |
| 7381 | ACTAGTTCCTGGGGATAAGAGTT | 8645 | AACTCTTATCCCCAGGAACTAGT |
| 7382 | AACTAGTTCCTGGGGATAAGAGT | 8646 | ACTCTTATCCCCAGGAACTAGTT |
| 7383 | AAACTAGTTCCTGGGGATAAGAG | 8647 | CTCTTATCCCCAGGAACTAGTTT |
| 7384 | CAAACTAGTTCCTGGGGATAAGA | 8648 | TCTTATCCCCAGGAACTAGTTTG |
| 7385 | ACAAACTAGTTCCTGGGGATAAG | 8649 | CTTATCCCCAGGAACTAGTTTGT |
| 7386 | AACAAACTAGTTCCTGGGGATAA | 8650 | TTATCCCCAGGAACTAGTTTGTT |
| 7387 | CAACAAACTAGTTCCTGGGGATA | 8651 | TATCCCCAGGAACTAGTTTGTTG |
| 7388 | TCAACAAACTAGTTCCTGGGGAT | 8652 | ATCCCCAGGAACTAGTTTGTTGA |
| 7389 | TTCAACAAACTAGTTCCTGGGGA | 8653 | TCCCCAGGAACTAGTTTGTTGAA |
| 7390 | ATTCAACAAACTAGTTCCTGGGG | 8654 | CCCCAGGAACTAGTTTGTTGAAT |
| 7391 | TATTCAACAAACTAGTTCCTGGG | 8655 | CCCAGGAACTAGTTTGTTGAATA |
| 7392 | TTATTCAACAAACTAGTTCCTGG | 8656 | CCAGGAACTAGTTTGTTGAATAA |
| 7393 | TTTATTCAACAAACTAGTTCCTG | 8657 | CAGGAACTAGTTTGTTGAATAAA |
| 7394 | ATTTATTCAACAAACTAGTTCCT | 8658 | AGGAACTAGTTTGTTGAATAAAT |
| 7395 | CATTTATTCAACAAACTAGTTCC | 8659 | GGAACTAGTTTGTTGAATAAATG |
| 7396 | GCATTTATTCAACAAACTAGTTC | 8660 | GAACTAGTTTGTTGAATAAATGC |
| 7397 | AGCATTTATTCAACAAACTAGTT | 8661 | AACTAGTTTGTTGAATAAATGCT |
| 7398 | CAGCATTTATTCAACAAACTAGT | 8662 | ACTAGTTTGTTGAATAAATGCTG |
| 7399 | CCAGCATTTATTCAACAAACTAG | 8663 | CTAGTTTGTTGAATAAATGCTGG |
| 7400 | ACCAGCATTTATTCAACAAACTA | 8664 | TAGTTTGTTGAATAAATGCTGGT |
| 7401 | CACCAGCATTTATTCAACAAACT | 8665 | AGTTTGTTGAATAAATGCTGGTG |
| 7402 | TCACCAGCATTTATTCAACAAAC | 8666 | GTTTGTTGAATAAATGCTGGTGA |
| 7403 | TTCACCAGCATTTATTCAACAAA | 8667 | TTTGTTGAATAAATGCTGGTGAA |
| 7404 | ATTCACCAGCATTTATTCAACAA | 8668 | TTGTTGAATAAATGCTGGTGAAT |
| 7405 | CATTCACCAGCATTTATTCAACA | 8669 | TGTTGAATAAATGCTGGTGAATG |
| 7406 | TCATTCACCAGCATTTATTCAAC | 8670 | GTTGAATAAATGCTGGTGAATGA |
| 7407 | TTCATTCACCAGCATTTATTCAA | 8671 | TTGAATAAATGCTGGTGAATGAA |
| 7408 | ATTCATTCACCAGCATTTATTCA | 8672 | TGAATAAATGCTGGTGAATGAAT |
| 7409 | CATTCATTCACCAGCATTTATTC | 8673 | GAATAAATGCTGGTGAATGAATG |
| 7410 | TCATTCATTCACCAGCATTTATT | 8674 | AATAAATGCTGGTGAATGAATGA |
| 7411 | TTCATTCATTCACCAGCATTTAT | 8675 | ATAAATGCTGGTGAATGAATGAA |
| 7412 | ATTCATTCATTCACCAGCATTTA | 8676 | TAAATGCTGGTGAATGAATGAAT |
| 7413 | CATTCATTCATTCACCAGCATTT | 8677 | AAATGCTGGTGAATGAATGAATG |
| 7414 | TCATTCATTCATTCACCAGCATT | 8678 | AATGCTGGTGAATGAATGAATGA |
| 7415 | ATCATTCATTCATTCACCAGCAT | 8679 | ATGCTGGTGAATGAATGAATGAT |
| 7416 | AATCATTCATTCATTCACCAGCA | 8680 | TGCTGGTGAATGAATGAATGATT |
| 7417 | CAATCATTCATTCATTCACCAGC | 8681 | GCTGGTGAATGAATGAATGATTG |
| 7418 | TCAATCATTCATTCATTCACCAG | 8682 | CTGGTGAATGAATGAATGATTGA |
| 7419 | TTCAATCATTCATTCATTCACCA | 8683 | TGGTGAATGAATGAATGATTGAA |
| 7420 | GTTCAATCATTCATTCATTCACC | 8684 | GGTGAATGAATGAATGATTGAAC |
| 7421 | TGTTCAATCATTCATTCATTCAC | 8685 | GTGAATGAATGAATGATTGAACA |
| 7422 | CTGTTCAATCATTCATTCATTCA | 8686 | TGAATGAATGAATGATTGAACAG |
| 7423 | TCTGTTCAATCATTCATTCATTC | 8687 | GAATGAATGAATGATTGAACAGA |
| 7424 | ATCTGTTCAATCATTCATTCATT | 8688 | AATGAATGAATGATTGAACAGAT |
| 7425 | CATCTGTTCAATCATTCATTCAT | 8689 | ATGAATGAATGATTGAACAGATG |
| 7426 | TCATCTGTTCAATCATTCATTCA | 8690 | TGAATGAATGATTGAACAGATGA |
| 7427 | TTCATCTGTTCAATCATTCATTC | 8691 | GAATGAATGATTGAACAGATGAA |
| 7428 | ATTCATCTGTTCAATCATTCATT | 8692 | AATGAATGATTGAACAGATGAAT |
| 7429 | CATTCATCTGTTCAATCATTCAT | 8693 | ATGAATGATTGAACAGATGAATG |
| 7430 | TCATTCATCTGTTCAATCATTCA | 8694 | TGAATGATTGAACAGATGAATGA |
| 7431 | CTCATTCATCTGTTCAATCATTC | 8695 | GAATGATTGAACAGATGAATGAG |
| 7432 | ACTCATTCATCTGTTCAATCATT | 8696 | AATGATTGAACAGATGAATGAGT |
| 7433 | CACTCATTCATCTGTTCAATCAT | 8697 | ATGATTGAACAGATGAATGAGTG |
| 7434 | TCACTCATTCATCTGTTCAATCA | 8698 | TGATTGAACAGATGAATGAGTGA |
| 7435 | ATCACTCATTCATCTGTTCAATC | 8699 | GATTGAACAGATGAATGAGTGAT |
| 7436 | CATCACTCATTCATCTGTTCAAT | 8700 | ATTGAACAGATGAATGAGTGATG |
| 7437 | TCATCACTCATTCATCTGTTCAA | 8701 | TTGAACAGATGAATGAGTGATGA |
| 7438 | CTCATCACTCATTCATCTGTTCA | 8702 | TGAACAGATGAATGAGTGATGAG |
| 7439 | ACTCATCACTCATTCATCTGTTC | 8703 | GAACAGATGAATGAGTGATGAGT |
| 7440 | TACTCATCACTCATTCATCTGTT | 8704 | AACAGATGAATGAGTGATGAGTA |
| 7441 | CTACTCATCACTCATTCATCTGT | 8705 | ACAGATGAATGAGTGATGAGTAG |
| 7442 | TCTACTCATCACTCATTCATCTG | 8706 | CAGATGAATGAGTGATGAGTAGA |
| 7443 | ATCTACTCATCACTCATTCATCT | 8707 | AGATGAATGAGTGATGAGTAGAT |
| 7444 | TATCTACTCATCACTCATTCATC | 8708 | GATGAATGAGTGATGAGTAGATA |
| 7445 | TTATCTACTCATCACTCATTCAT | 8709 | ATGAATGAGTGATGAGTAGATAA |
| 7446 | TTTATCTACTCATCACTCATTCA | 8710 | TGAATGAGTGATGAGTAGATAAA |
| 7447 | TTTTATCTACTCATCACTCATTC | 8711 | GAATGAGTGATGAGTAGATAAAA |
| 7448 | CTTTTATCTACTCATCACTCATT | 8712 | AATGAGTGATGAGTAGATAAAAG |
| 7449 | CCTTTTATCTACTCATCACTCAT | 8713 | ATGAGTGATGAGTAGATAAAAGG |
| 7450 | TCCTTTTATCTACTCATCACTCA | 8714 | TGAGTGATGAGTAGATAAAAGGA |
| 7451 | ATCCTTTTATCTACTCATCACTC | 8715 | GAGTGATGAGTAGATAAAAGGAT |
| 7452 | CATCCTTTTATCTACTCATCACT | 8716 | AGTGATGAGTAGATAAAAGGATG |
| 7453 | CCATCCTTTTATCTACTCATCAC | 8717 | GTGATGAGTAGATAAAAGGATGG |
| 7454 | TCCATCCTTTTATCTACTCATCA | 8718 | TGATGAGTAGATAAAAGGATGGA |
| 7455 | ATCCATCCTTTTATCTACTCATC | 8719 | GATGAGTAGATAAAAGGATGGAT |
| 7456 | CATCCATCCTTTTATCTACTCAT | 8720 | ATGAGTAGATAAAAGGATGGATG |
| 7457 | CCATCCATCCTTTTATCTACTCA | 8721 | TGAGTAGATAAAAGGATGGATGG |
| 7458 | TCCATCCATCCTTTTATCTACTC | 8722 | GAGTAGATAAAAGGATGGATGGA |
| 7459 | CTCCATCCATCCTTTTATCTACT | 8723 | AGTAGATAAAAGGATGGATGGAG |
| 7460 | TCTCCATCCATCCTTTTATCTAC | 8724 | GTAGATAAAAGGATGGATGGAGA |
| 7461 | CTCTCCATCCATCCTTTTATCTA | 8725 | TAGATAAAAGGATGGATGGAGAG |
| 7462 | TCTCTCCATCCATCCTTTTATCT | 8726 | AGATAAAAGGATGGATGGAGAGA |
| 7463 | ATCTCTCCATCCATCCTTTTATC | 8727 | GATAAAAGGATGGATGGAGAGAT |
| 7464 | CATCTCTCCATCCATCCTTTTAT | 8728 | ATAAAAGGATGGATGGAGAGATG |
| 7465 | CCATCTCTCCATCCATCCTTTTA | 8729 | TAAAAGGATGGATGGAGAGATGG |
| 7466 | CCCATCTCTCCATCCATCCTTTT | 8730 | AAAAGGATGGATGGAGAGATGGG |
| 7467 | ACCCATCTCTCCATCCATCCTTT | 8731 | AAAGGATGGATGGAGAGATGGGT |
| 7468 | CACCCATCTCTCCATCCATCCTT | 8732 | AAGGATGGATGGAGAGATGGGTG |
| 7469 | TCACCCATCTCTCCATCCATCCT | 8733 | AGGATGGATGGAGAGATGGGTGA |
| 7470 | CTCACCCATCTCTCCATCCATCC | 8734 | GGATGGATGGAGAGATGGGTGAG |
| 7471 | ACTCACCCATCTCTCCATCCATC | 8735 | GATGGATGGAGAGATGGGTGAGT |
| 7472 | TACTCACCCATCTCTCCATCCAT | 8736 | ATGGATGGAGAGATGGGTGAGTA |
| 7473 | GTACTCACCCATCTCTCCATCCA | 8737 | TGGATGGAGAGATGGGTGAGTAC |
| 7474 | TGTACTCACCCATCTCTCCATCC | 8738 | GGATGGAGAGATGGGTGAGTACA |
| 7475 | ATGTACTCACCCATCTCTCCATC | 8739 | GATGGAGAGATGGGTGAGTACAT |
| 7476 | CATGTACTCACCCATCTCTCCAT | 8740 | ATGGAGAGATGGGTGAGTACATG |
| 7477 | CCATGTACTCACCCATCTCTCCA | 8741 | TGGAGAGATGGGTGAGTACATGG |
| 7478 | TCCATGTACTCACCCATCTCTCC | 8742 | GGAGAGATGGGTGAGTACATGGA |
| 7479 | ATCCATGTACTCACCCATCTCTC | 8743 | GAGAGATGGGTGAGTACATGGAT |
| 7480 | CATCCATGTACTCACCCATCTCT | 8744 | AGAGATGGGTGAGTACATGGATG |
| 7481 | CCATCCATGTACTCACCCATCTC | 8745 | GAGATGGGTGAGTACATGGATGG |
| 7482 | TCCATCCATGTACTCACCCATCT | 8746 | AGATGGGTGAGTACATGGATGGA |
| 7483 | ATCCATCCATGTACTCACCCATC | 8747 | GATGGGTGAGTACATGGATGGAT |
| 7484 | TATCCATCCATGTACTCACCCAT | 8748 | ATGGGTGAGTACATGGATGGATA |
| 7485 | CTATCCATCCATGTACTCACCCA | 8749 | TGGGTGAGTACATGGATGGATAG |
| 7486 | TCTATCCATCCATGTACTCACCC | 8750 | GGGTGAGTACATGGATGGATAGA |
| 7487 | ATCTATCCATCCATGTACTCACC | 8751 | GGTGAGTACATGGATGGATAGAT |
| 7488 | CATCTATCCATCCATGTACTCAC | 8752 | GTGAGTACATGGATGGATAGATG |
| 7489 | CCATCTATCCATCCATGTACTCA | 8753 | TGAGTACATGGATGGATAGATGG |
| 7490 | TCCATCTATCCATCCATGTACTC | 8754 | GAGTACATGGATGGATAGATGGA |
| 7491 | ATCCATCTATCCATCCATGTACT | 8755 | AGTACATGGATGGATAGATGGAT |
| 7492 | CATCCATCTATCCATCCATGTAC | 8756 | GTACATGGATGGATAGATGGATG |
| 7493 | TCATCCATCTATCCATCCATGTA | 8757 | TACATGGATGGATAGATGGATGA |
| 7494 | CTCATCCATCTATCCATCCATGT | 8758 | ACATGGATGGATAGATGGATGAG |
| 7495 | ACTCATCCATCTATCCATCCATG | 8759 | CATGGATGGATAGATGGATGAGT |
| 7496 | AACTCATCCATCTATCCATCCAT | 8760 | ATGGATGGATAGATGGATGAGTT |
| 7497 | CAACTCATCCATCTATCCATCCA | 8761 | TGGATGGATAGATGGATGAGTTG |
| 7498 | CCAACTCATCCATCTATCCATCC | 8762 | GGATGGATAGATGGATGAGTTGG |
| 7499 | ACCAACTCATCCATCTATCCATC | 8763 | GATGGATAGATGGATGAGTTGGT |
| 7500 | CACCAACTCATCCATCTATCCAT | 8764 | ATGGATAGATGGATGAGTTGGTG |
| 7501 | CCACCAACTCATCCATCTATCCA | 8765 | TGGATAGATGGATGAGTTGGTGG |
| 7502 | CCCACCAACTCATCCATCTATCC | 8766 | GGATAGATGGATGAGTTGGTGGG |
| 7503 | ACCCACCAACTCATCCATCTATC | 8767 | GATAGATGGATGAGTTGGTGGGT |
| 7504 | TACCCACCAACTCATCCATCTAT | 8768 | ATAGATGGATGAGTTGGTGGGTA |
| 7505 | CTACCCACCAACTCATCCATCTA | 8769 | TAGATGGATGAGTTGGTGGGTAG |
| 7506 | TCTACCCACCAACTCATCCATCT | 8770 | AGATGGATGAGTTGGTGGGTAGA |
| 7507 | ATCTACCCACCAACTCATCCATC | 8771 | GATGGATGAGTTGGTGGGTAGAT |
| 7508 | AATCTACCCACCAACTCATCCAT | 8772 | ATGGATGAGTTGGTGGGTAGATT |
| 7509 | GAATCTACCCACCAACTCATCCA | 8773 | TGGATGAGTTGGTGGGTAGATTC |
| 7510 | CGAATCTACCCACCAACTCATCC | 8774 | GGATGAGTTGGTGGGTAGATTCG |
| 7511 | ACGAATCTACCCACCAACTCATC | 8775 | GATGAGTTGGTGGGTAGATTCGT |
| 7512 | CACGAATCTACCCACCAACTCAT | 8776 | ATGAGTTGGTGGGTAGATTCGTG |
| 7513 | CCACGAATCTACCCACCAACTCA | 8777 | TGAGTTGGTGGGTAGATTCGTGG |
| 7514 | GCCACGAATCTACCCACCAACTC | 8778 | GAGTTGGTGGGTAGATTCGTGGC |
| 7515 | AGCCACGAATCTACCCACCAACT | 8779 | AGTTGGTGGGTAGATTCGTGGCT |
| 7516 | TAGCCACGAATCTACCCACCAAC | 8780 | GTTGGTGGGTAGATTCGTGGCTA |
| 7517 | CTAGCCACGAATCTACCCACCAA | 8781 | TTGGTGGGTAGATTCGTGGCTAG |
| 7518 | TCTAGCCACGAATCTACCCACCA | 8782 | TGGTGGGTAGATTCGTGGCTAGA |
| 7519 | ATCTAGCCACGAATCTACCCACC | 8783 | GGTGGGTAGATTCGTGGCTAGAT |
| 7520 | CATCTAGCCACGAATCTACCCAC | 8784 | GTGGGTAGATTCGTGGCTAGATG |
| 7521 | CCATCTAGCCACGAATCTACCCA | 8785 | TGGGTAGATTCGTGGCTAGATGG |
| 7522 | TCCATCTAGCCACGAATCTACCC | 8786 | GGGTAGATTCGTGGCTAGATGGA |
| 7523 | ATCCATCTAGCCACGAATCTACC | 8787 | GGTAGATTCGTGGCTAGATGGAT |
| 7524 | CATCCATCTAGCCACGAATCTAC | 8788 | GTAGATTCGTGGCTAGATGGATG |
| 7525 | TCATCCATCTAGCCACGAATCTA | 8789 | TAGATTCGTGGCTAGATGGATGA |
| 7526 | ATCATCCATCTAGCCACGAATCT | 8790 | AGATTCGTGGCTAGATGGATGAT |
| 7527 | CATCATCCATCTAGCCACGAATC | 8791 | GATTCGTGGCTAGATGGATGATG |
| 7528 | CCATCATCCATCTAGCCACGAAT | 8792 | ATTCGTGGCTAGATGGATGATGG |
| 7529 | TCCATCATCCATCTAGCCACGAA | 8793 | TTCGTGGCTAGATGGATGATGGA |
| 7530 | ATCCATCATCCATCTAGCCACGA | 8794 | TCGTGGCTAGATGGATGATGGAT |
| 7531 | CATCCATCATCCATCTAGCCACG | 8795 | CGTGGCTAGATGGATGATGGATG |
| 7532 | CCATCCATCATCCATCTAGCCAC | 8796 | GTGGCTAGATGGATGATGGATGG |
| 7533 | TCCATCCATCATCCATCTAGCCA | 8797 | TGGCTAGATGGATGATGGATGGA |
| 7534 | ATCCATCCATCATCCATCTAGCC | 8798 | GGCTAGATGGATGATGGATGGAT |
| 7535 | CATCCATCCATCATCCATCTAGC | 8799 | GCTAGATGGATGATGGATGGATG |
| 7536 | CCATCCATCCATCATCCATCTAG | 8800 | CTAGATGGATGATGGATGGATGG |
| 7537 | TCCATCCATCCATCATCCATCTA | 8801 | TAGATGGATGATGGATGGATGGA |
| 7538 | GTCCATCCATCCATCATCCATCT | 8802 | AGATGGATGATGGATGGATGGAC |
| 7539 | TGTCCATCCATCCATCATCCATC | 8803 | GATGGATGATGGATGGATGGACA |
| 7540 | CTGTCCATCCATCCATCATCCAT | 8804 | ATGGATGATGGATGGATGGACAG |
| 7541 | TCTGTCCATCCATCCATCATCCA | 8805 | TGGATGATGGATGGATGGACAGA |
| 7542 | ATCTGTCCATCCATCCATCATCC | 8806 | GGATGATGGATGGATGGACAGAT |
| 7543 | CATCTGTCCATCCATCCATCATC | 8807 | GATGATGGATGGATGGACAGATG |
| 7544 | CCATCTGTCCATCCATCCATCAT | 8808 | ATGATGGATGGATGGACAGATGG |
| 7545 | TCCATCTGTCCATCCATCCATCA | 8809 | TGATGGATGGATGGACAGATGGA |
| 7546 | ATCCATCTGTCCATCCATCCATC | 8810 | GATGGATGGATGGACAGATGGAT |
| 7547 | CATCCATCTGTCCATCCATCCAT | 8811 | ATGGATGGATGGACAGATGGATG |
| 7548 | CCATCCATCTGTCCATCCATCCA | 8812 | TGGATGGATGGACAGATGGATGG |
| 7549 | TCCATCCATCTGTCCATCCATCC | 8813 | GGATGGATGGACAGATGGATGGA |
| 7550 | ATCCATCCATCTGTCCATCCATC | 8814 | GATGGATGGACAGATGGATGGAT |
| 7551 | TATCCATCCATCTGTCCATCCAT | 8815 | ATGGATGGACAGATGGATGGATA |
| 7552 | ATATCCATCCATCTGTCCATCCA | 8816 | TGGATGGACAGATGGATGGATAT |
| 7553 | TATATCCATCCATCTGTCCATCC | 8817 | GGATGGACAGATGGATGGATATA |
| 7554 | ATATATCCATCCATCTGTCCATC | 8818 | GATGGACAGATGGATGGATATAT |
| 7555 | CATATATCCATCCATCTGTCCAT | 8819 | ATGGACAGATGGATGGATATATG |
| 7556 | TCATATATCCATCCATCTGTCCA | 8820 | TGGACAGATGGATGGATATATGA |
| 7557 | ATCATATATCCATCCATCTGTCC | 8821 | GGACAGATGGATGGATATATGAT |
| 7558 | AATCATATATCCATCCATCTGTC | 8822 | GACAGATGGATGGATATATGATT |
| 7559 | CAATCATATATCCATCCATCTGT | 8823 | ACAGATGGATGGATATATGATTG |
| 7560 | TCAATCATATATCCATCCATCTG | 8824 | CAGATGGATGGATATATGATTGA |
| 7561 | TTCAATCATATATCCATCCATCT | 8825 | AGATGGATGGATATATGATTGAA |
| 7562 | GTTCAATCATATATCCATCCATC | 8826 | GATGGATGGATATATGATTGAAC |
| 7563 | AGTTCAATCATATATCCATCCAT | 8827 | ATGGATGGATATATGATTGAACT |
| 7564 | TAGTTCAATCATATATCCATCCA | 8828 | TGGATGGATATATGATTGAACTA |
| 7565 | ATAGTTCAATCATATATCCATCC | 8829 | GGATGGATATATGATTGAACTAT |
| 7566 | AATAGTTCAATCATATATCCATC | 8830 | GATGGATATATGATTGAACTATT |
| 7567 | CAATAGTTCAATCATATATCCAT | 8831 | ATGGATATATGATTGAACTATTG |
| 7568 | TCAATAGTTCAATCATATATCCA | 8832 | TGGATATATGATTGAACTATTGA |
| 7569 | TTCAATAGTTCAATCATATATCC | 8833 | GGATATATGATTGAACTATTGAA |
| 7570 | TTTCAATAGTTCAATCATATATC | 8834 | GATATATGATTGAACTATTGAAA |
| 7571 | CTTTCAATAGTTCAATCATATAT | 8835 | ATATATGATTGAACTATTGAAAG |
| 7572 | ACTTTCAATAGTTCAATCATATA | 8836 | TATATGATTGAACTATTGAAAGT |
| 7573 | TACTTTCAATAGTTCAATCATAT | 8837 | ATATGATTGAACTATTGAAAGTA |
| 7574 | ATACTTTCAATAGTTCAATCATA | 8838 | TATGATTGAACTATTGAAAGTAT |
| 7575 | TATACTTTCAATAGTTCAATCAT | 8839 | ATGATTGAACTATTGAAAGTATA |
| 7576 | CTATACTTTCAATAGTTCAATCA | 8840 | TGATTGAACTATTGAAAGTATAG |
| 7577 | TCTATACTTTCAATAGTTCAATC | 8841 | GATTGAACTATTGAAAGTATAGA |
| 7578 | ATCTATACTTTCAATAGTTCAAT | 8842 | ATTGAACTATTGAAAGTATAGAT |
| 7579 | CATCTATACTTTCAATAGTTCAA | 8843 | TTGAACTATTGAAAGTATAGATG |
| 7580 | ACATCTATACTTTCAATAGTTCA | 8844 | TGAACTATTGAAAGTATAGATGT |
| 7581 | TACATCTATACTTTCAATAGTTC | 8845 | GAACTATTGAAAGTATAGATGTA |
| 7582 | ATACATCTATACTTTCAATAGTT | 8846 | AACTATTGAAAGTATAGATGTAT |
| 7583 | CATACATCTATACTTTCAATAGT | 8847 | ACTATTGAAAGTATAGATGTATG |
| 7584 | CCATACATCTATACTTTCAATAG | 8848 | CTATTGAAAGTATAGATGTATGG |
| 7585 | TCCATACATCTATACTTTCAATA | 8849 | TATTGAAAGTATAGATGTATGGA |
| 7586 | ATCCATACATCTATACTTTCAAT | 8850 | ATTGAAAGTATAGATGTATGGAT |
| 7587 | CATCCATACATCTATACTTTCAA | 8851 | TTGAAAGTATAGATGTATGGATG |
| 7588 | CCATCCATACATCTATACTTTCA | 8852 | TGAAAGTATAGATGTATGGATGG |
| 7589 | CCCATCCATACATCTATACTTTC | 8853 | GAAAGTATAGATGTATGGATGGG |
| 7590 | ACCCATCCATACATCTATACTTT | 8854 | AAAGTATAGATGTATGGATGGGT |
| 7591 | CACCCATCCATACATCTATACTT | 8855 | AAGTATAGATGTATGGATGGGTG |
| 7592 | TCACCCATCCATACATCTATACT | 8856 | AGTATAGATGTATGGATGGGTGA |
| 7593 | TTCACCCATCCATACATCTATAC | 8857 | GTATAGATGTATGGATGGGTGAA |
| 7594 | ATTCACCCATCCATACATCTATA | 8858 | TATAGATGTATGGATGGGTGAAT |
| 7595 | AATTCACCCATCCATACATCTAT | 8859 | ATAGATGTATGGATGGGTGAATT |
| 7596 | AAATTCACCCATCCATACATCTA | 8860 | TAGATGTATGGATGGGTGAATTT |
| 7597 | CAAATTCACCCATCCATACATCT | 8861 | AGATGTATGGATGGGTGAATTTG |
| 7598 | CCAAATTCACCCATCCATACATC | 8862 | GATGTATGGATGGGTGAATTTGG |
| 7599 | CCCAAATTCACCCATCCATACAT | 8863 | ATGTATGGATGGGTGAATTTGGG |
| 7600 | CCCCAAATTCACCCATCCATACA | 8864 | TGTATGGATGGGTGAATTTGGGG |
| 7601 | CCCCCAAATTCACCCATCCATAC | 8865 | GTATGGATGGGTGAATTTGGGGG |
| 7602 | ACCCCCAAATTCACCCATCCATA | 8866 | TATGGATGGGTGAATTTGGGGGT |
| 7603 | TACCCCCAAATTCACCCATCCAT | 8867 | ATGGATGGGTGAATTTGGGGGTA |
| 7604 | TTACCCCCAAATTCACCCATCCA | 8868 | TGGATGGGTGAATTTGGGGGTAA |
| 7605 | ATTACCCCCAAATTCACCCATCC | 8869 | GGATGGGTGAATTTGGGGGTAAT |
| 7606 | AATTACCCCCAAATTCACCCATC | 8870 | GATGGGTGAATTTGGGGGTAATT |
| 7607 | CAATTACCCCCAAATTCACCCAT | 8871 | ATGGGTGAATTTGGGGGTAATTG |
| 7608 | ACAATTACCCCCAAATTCACCCA | 8872 | TGGGTGAATTTGGGGGTAATTGT |
| 7609 | AACAATTACCCCCAAATTCACCC | 8873 | GGGTGAATTTGGGGGTAATTGTT |
| 7610 | TAACAATTACCCCCAAATTCACC | 8874 | GGTGAATTTGGGGGTAATTGTTA |
| 7611 | CTAACAATTACCCCCAAATTCAC | 8875 | GTGAATTTGGGGGTAATTGTTAG |
| 7612 | TCTAACAATTACCCCCAAATTCA | 8876 | TGAATTTGGGGGTAATTGTTAGA |
| 7613 | ATCTAACAATTACCCCCAAATTC | 8877 | GAATTTGGGGGTAATTGTTAGAT |
| 7614 | CATCTAACAATTACCCCCAAATT | 8878 | AATTTGGGGGTAATTGTTAGATG |
| 7615 | TCATCTAACAATTACCCCCAAAT | 8879 | ATTTGGGGGTAATTGTTAGATGA |
| 7616 | ATCATCTAACAATTACCCCCAAA | 8880 | TTTGGGGGTAATTGTTAGATGAT |
| 7617 | CATCATCTAACAATTACCCCCAA | 8881 | TTGGGGGTAATTGTTAGATGATG |
| 7618 | CCATCATCTAACAATTACCCCCA | 8882 | TGGGGGTAATTGTTAGATGATGG |
| 7619 | TCCATCATCTAACAATTACCCCC | 8883 | GGGGGTAATTGTTAGATGATGGA |
| 7620 | ATCCATCATCTAACAATTACCCC | 8884 | GGGGTAATTGTTAGATGATGGAT |
| 7621 | CATCCATCATCTAACAATTACCC | 8885 | GGGTAATTGTTAGATGATGGATG |
| 7622 | TCATCCATCATCTAACAATTACC | 8886 | GGTAATTGTTAGATGATGGATGA |
| 7623 | CTCATCCATCATCTAACAATTAC | 8887 | GTAATTGTTAGATGATGGATGAG |
| 7624 | ACTCATCCATCATCTAACAATTA | 8888 | TAATTGTTAGATGATGGATGAGT |
| 7625 | TACTCATCCATCATCTAACAATT | 8889 | AATTGTTAGATGATGGATGAGTA |
| 7626 | ATACTCATCCATCATCTAACAAT | 8890 | ATTGTTAGATGATGGATGAGTAT |
| 7627 | TATACTCATCCATCATCTAACAA | 8891 | TTGTTAGATGATGGATGAGTATA |
| 7628 | CTATACTCATCCATCATCTAACA | 8892 | TGTTAGATGATGGATGAGTATAG |
| 7629 | TCTATACTCATCCATCATCTAAC | 8893 | GTTAGATGATGGATGAGTATAGA |
| 7630 | ATCTATACTCATCCATCATCTAA | 8894 | TTAGATGATGGATGAGTATAGAT |
| 7631 | CATCTATACTCATCCATCATCTA | 8895 | TAGATGATGGATGAGTATAGATG |
| 7632 | TCATCTATACTCATCCATCATCT | 8896 | AGATGATGGATGAGTATAGATGA |
| 7633 | TTCATCTATACTCATCCATCATC | 8897 | GATGATGGATGAGTATAGATGAA |
| 7634 | ATTCATCTATACTCATCCATCAT | 8898 | ATGATGGATGAGTATAGATGAAT |
| 7635 | CATTCATCTATACTCATCCATCA | 8899 | TGATGGATGAGTATAGATGAATG |
| 7636 | TCATTCATCTATACTCATCCATC | 8900 | GATGGATGAGTATAGATGAATGA |
| 7637 | ATCATTCATCTATACTCATCCAT | 8901 | ATGGATGAGTATAGATGAATGAT |
| 7638 | CATCATTCATCTATACTCATCCA | 8902 | TGGATGAGTATAGATGAATGATG |
| 7639 | CCATCATTCATCTATACTCATCC | 8903 | GGATGAGTATAGATGAATGATGG |
| 7640 | TCCATCATTCATCTATACTCATC | 8904 | GATGAGTATAGATGAATGATGGA |
| 7641 | ATCCATCATTCATCTATACTCAT | 8905 | ATGAGTATAGATGAATGATGGAT |
| 7642 | CATCCATCATTCATCTATACTCA | 8906 | TGAGTATAGATGAATGATGGATG |
| 7643 | CCATCCATCATTCATCTATACTC | 8907 | GAGTATAGATGAATGATGGATGG |
| 7644 | TCCATCCATCATTCATCTATACT | 8908 | AGTATAGATGAATGATGGATGGA |
| 7645 | ATCCATCCATCATTCATCTATAC | 8909 | GTATAGATGAATGATGGATGGAT |
| 7646 | TATCCATCCATCATTCATCTATA | 8910 | TATAGATGAATGATGGATGGATA |
| 7647 | TTATCCATCCATCATTCATCTAT | 8911 | ATAGATGAATGATGGATGGATAA |
| 7648 | GTTATCCATCCATCATTCATCTA | 8912 | TAGATGAATGATGGATGGATAAC |
| 7649 | AGTTATCCATCCATCATTCATCT | 8913 | AGATGAATGATGGATGGATAACT |
| 7650 | AAGTTATCCATCCATCATTCATC | 8914 | GATGAATGATGGATGGATAACTT |
| 7651 | CAAGTTATCCATCCATCATTCAT | 8915 | ATGAATGATGGATGGATAACTTG |
| 7652 | TCAAGTTATCCATCCATCATTCA | 8916 | TGAATGATGGATGGATAACTTGA |
| 7653 | ATCAAGTTATCCATCCATCATTC | 8917 | GAATGATGGATGGATAACTTGAT |
| 7654 | CATCAAGTTATCCATCCATCATT | 8918 | AATGATGGATGGATAACTTGATG |
| 7655 | TCATCAAGTTATCCATCCATCAT | 8919 | ATGATGGATGGATAACTTGATGA |
| 7656 | CTCATCAAGTTATCCATCCATCA | 8920 | TGATGGATGGATAACTTGATGAG |
| 7657 | ACTCATCAAGTTATCCATCCATC | 8921 | GATGGATGGATAACTTGATGAGT |
| 7658 | CACTCATCAAGTTATCCATCCAT | 8922 | ATGGATGGATAACTTGATGAGTG |
| 7659 | CCACTCATCAAGTTATCCATCCA | 8923 | TGGATGGATAACTTGATGAGTGG |
| 7660 | TCCACTCATCAAGTTATCCATCC | 8924 | GGATGGATAACTTGATGAGTGGA |
| 7661 | ATCCACTCATCAAGTTATCCATC | 8925 | GATGGATAACTTGATGAGTGGAT |
| 7662 | TATCCACTCATCAAGTTATCCAT | 8926 | ATGGATAACTTGATGAGTGGATA |
| 7663 | CTATCCACTCATCAAGTTATCCA | 8927 | TGGATAACTTGATGAGTGGATAG |
| 7664 | TCTATCCACTCATCAAGTTATCC | 8928 | GGATAACTTGATGAGTGGATAGA |
| 7665 | ATCTATCCACTCATCAAGTTATC | 8929 | GATAACTTGATGAGTGGATAGAT |
| 7666 | TATCTATCCACTCATCAAGTTAT | 8930 | ATAACTTGATGAGTGGATAGATA |
| 7667 | CTATCTATCCACTCATCAAGTTA | 8931 | TAACTTGATGAGTGGATAGATAG |
| 7668 | TCTATCTATCCACTCATCAAGTT | 8932 | AACTTGATGAGTGGATAGATAGA |
| 7669 | ATCTATCTATCCACTCATCAAGT | 8933 | ACTTGATGAGTGGATAGATAGAT |
| 7670 | AATCTATCTATCCACTCATCAAG | 8934 | CTTGATGAGTGGATAGATAGATT |
| 7671 | CAATCTATCTATCCACTCATCAA | 8935 | TTGATGAGTGGATAGATAGATTG |
| 7672 | GCAATCTATCTATCCACTCATCA | 8936 | TGATGAGTGGATAGATAGATTGC |
| 7673 | AGCAATCTATCTATCCACTCATC | 8937 | GATGAGTGGATAGATAGATTGCT |
| 7674 | CAGCAATCTATCTATCCACTCAT | 8938 | ATGAGTGGATAGATAGATTGCTG |
| 7675 | CCAGCAATCTATCTATCCACTCA | 8939 | TGAGTGGATAGATAGATTGCTGG |
| 7676 | TCCAGCAATCTATCTATCCACTC | 8940 | GAGTGGATAGATAGATTGCTGGA |
| 7677 | ATCCAGCAATCTATCTATCCACT | 8941 | AGTGGATAGATAGATTGCTGGAT |
| 7678 | TATCCAGCAATCTATCTATCCAC | 8942 | GTGGATAGATAGATTGCTGGATA |
| 7679 | CTATCCAGCAATCTATCTATCCA | 8943 | TGGATAGATAGATTGCTGGATAG |
| 7680 | TCTATCCAGCAATCTATCTATCC | 8944 | GGATAGATAGATTGCTGGATAGA |
| 7681 | ATCTATCCAGCAATCTATCTATC | 8945 | GATAGATAGATTGCTGGATAGAT |
| 7682 | CATCTATCCAGCAATCTATCTAT | 8946 | ATAGATAGATTGCTGGATAGATG |
| 7683 | TCATCTATCCAGCAATCTATCTA | 8947 | TAGATAGATTGCTGGATAGATGA |
| 7684 | ATCATCTATCCAGCAATCTATCT | 8948 | AGATAGATTGCTGGATAGATGAT |
| 7685 | AATCATCTATCCAGCAATCTATC | 8949 | GATAGATTGCTGGATAGATGATT |
| 7686 | CAATCATCTATCCAGCAATCTAT | 8950 | ATAGATTGCTGGATAGATGATTG |
| 7687 | TCAATCATCTATCCAGCAATCTA | 8951 | TAGATTGCTGGATAGATGATTGA |
| 7688 | GTCAATCATCTATCCAGCAATCT | 8952 | AGATTGCTGGATAGATGATTGAC |
| 7689 | AGTCAATCATCTATCCAGCAATC | 8953 | GATTGCTGGATAGATGATTGACT |
| 7690 | CAGTCAATCATCTATCCAGCAAT | 8954 | ATTGCTGGATAGATGATTGACTG |
| 7691 | CCAGTCAATCATCTATCCAGCAA | 8955 | TTGCTGGATAGATGATTGACTGG |
| 7692 | CCCAGTCAATCATCTATCCAGCA | 8956 | TGCTGGATAGATGATTGACTGGG |
| 7693 | ACCCAGTCAATCATCTATCCAGC | 8957 | GCTGGATAGATGATTGACTGGGT |
| 7694 | CACCCAGTCAATCATCTATCCAG | 8958 | CTGGATAGATGATTGACTGGGTG |
| 7695 | CCACCCAGTCAATCATCTATCCA | 8959 | TGGATAGATGATTGACTGGGTGG |
| 7696 | TCCACCCAGTCAATCATCTATCC | 8960 | GGATAGATGATTGACTGGGTGGA |
| 7697 | ATCCACCCAGTCAATCATCTATC | 8961 | GATAGATGATTGACTGGGTGGAT |
| 7698 | TATCCACCCAGTCAATCATCTAT | 8962 | ATAGATGATTGACTGGGTGGATA |
| 7699 | CTATCCACCCAGTCAATCATCTA | 8963 | TAGATGATTGACTGGGTGGATAG |
| 7700 | TCTATCCACCCAGTCAATCATCT | 8964 | AGATGATTGACTGGGTGGATAGA |
| 7701 | ATCTATCCACCCAGTCAATCATC | 8965 | GATGATTGACTGGGTGGATAGAT |
| 7702 | CATCTATCCACCCAGTCAATCAT | 8966 | ATGATTGACTGGGTGGATAGATG |
| 7703 | TCATCTATCCACCCAGTCAATCA | 8967 | TGATTGACTGGGTGGATAGATGA |
| 7704 | TTCATCTATCCACCCAGTCAATC | 8968 | GATTGACTGGGTGGATAGATGAA |
| 7705 | TTTCATCTATCCACCCAGTCAAT | 8969 | ATTGACTGGGTGGATAGATGAAA |
| 7706 | ATTTCATCTATCCACCCAGTCAA | 8970 | TTGACTGGGTGGATAGATGAAAT |
| 7707 | CATTTCATCTATCCACCCAGTCA | 8971 | TGACTGGGTGGATAGATGAAATG |
| 7708 | ACATTTCATCTATCCACCCAGTC | 8972 | GACTGGGTGGATAGATGAAATGT |
| 7709 | AACATTTCATCTATCCACCCAGT | 8973 | ACTGGGTGGATAGATGAAATGTT |
| 7710 | CAACATTTCATCTATCCACCCAG | 8974 | CTGGGTGGATAGATGAAATGTTG |
| 7711 | CCAACATTTCATCTATCCACCCA | 8975 | TGGGTGGATAGATGAAATGTTGG |
| 7712 | TCCAACATTTCATCTATCCACCC | 8976 | GGGTGGATAGATGAAATGTTGGA |
| 7713 | ATCCAACATTTCATCTATCCACC | 8977 | GGTGGATAGATGAAATGTTGGAT |
| 7714 | CATCCAACATTTCATCTATCCAC | 8978 | GTGGATAGATGAAATGTTGGATG |
| 7715 | TCATCCAACATTTCATCTATCCA | 8979 | TGGATAGATGAAATGTTGGATGA |
| 7716 | CTCATCCAACATTTCATCTATCC | 8980 | GGATAGATGAAATGTTGGATGAG |
| 7717 | GCTCATCCAACATTTCATCTATC | 8981 | GATAGATGAAATGTTGGATGAGC |
| 7718 | TGCTCATCCAACATTTCATCTAT | 8982 | ATAGATGAAATGTTGGATGAGCA |
| 7719 | CTGCTCATCCAACATTTCATCTA | 8983 | TAGATGAAATGTTGGATGAGCAG |
| 7720 | TCTGCTCATCCAACATTTCATCT | 8984 | AGATGAAATGTTGGATGAGCAGA |
| 7721 | ATCTGCTCATCCAACATTTCATC | 8985 | GATGAAATGTTGGATGAGCAGAT |
| 7722 | AATCTGCTCATCCAACATTTCAT | 8986 | ATGAAATGTTGGATGAGCAGATT |
| 7723 | TAATCTGCTCATCCAACATTTCA | 8987 | TGAAATGTTGGATGAGCAGATTA |
| 7724 | TTAATCTGCTCATCCAACATTTC | 8988 | GAAATGTTGGATGAGCAGATTAA |
| 7725 | CTTAATCTGCTCATCCAACATTT | 8989 | AAATGTTGGATGAGCAGATTAAG |
| 7726 | ACTTAATCTGCTCATCCAACATT | 8990 | AATGTTGGATGAGCAGATTAAGT |
| 7727 | AACTTAATCTGCTCATCCAACAT | 8991 | ATGTTGGATGAGCAGATTAAGTT |
| 7728 | CAACTTAATCTGCTCATCCAACA | 8992 | TGTTGGATGAGCAGATTAAGTTG |
| 7729 | ACAACTTAATCTGCTCATCCAAC | 8993 | GTTGGATGAGCAGATTAAGTTGT |
| 7730 | TACAACTTAATCTGCTCATCCAA | 8994 | TTGGATGAGCAGATTAAGTTGTA |
| 7731 | ATACAACTTAATCTGCTCATCCA | 8995 | TGGATGAGCAGATTAAGTTGTAT |
| 7732 | AATACAACTTAATCTGCTCATCC | 8996 | GGATGAGCAGATTAAGTTGTATT |
| 7733 | CAATACAACTTAATCTGCTCATC | 8997 | GATGAGCAGATTAAGTTGTATTG |
| 7734 | CCAATACAACTTAATCTGCTCAT | 8998 | ATGAGCAGATTAAGTTGTATTGG |
| 7735 | TCCAATACAACTTAATCTGCTCA | 8999 | TGAGCAGATTAAGTTGTATTGGA |
| 7736 | ATCCAATACAACTTAATCTGCTC | 9000 | GAGCAGATTAAGTTGTATTGGAT |
| 7737 | CATCCAATACAACTTAATCTGCT | 9001 | AGCAGATTAAGTTGTATTGGATG |
| 7738 | CCATCCAATACAACTTAATCTGC | 9002 | GCAGATTAAGTTGTATTGGATGG |
| 7739 | CCCATCCAATACAACTTAATCTG | 9003 | CAGATTAAGTTGTATTGGATGGG |
| 7740 | TCCCATCCAATACAACTTAATCT | 9004 | AGATTAAGTTGTATTGGATGGGA |
| 7741 | ATCCCATCCAATACAACTTAATC | 9005 | GATTAAGTTGTATTGGATGGGAT |
| 7742 | CATCCCATCCAATACAACTTAAT | 9006 | ATTAAGTTGTATTGGATGGGATG |
| 7743 | CCATCCCATCCAATACAACTTAA | 9007 | TTAAGTTGTATTGGATGGGATGG |
| 7744 | TCCATCCCATCCAATACAACTTA | 9008 | TAAGTTGTATTGGATGGGATGGA |
| 7745 | ATCCATCCCATCCAATACAACTT | 9009 | AAGTTGTATTGGATGGGATGGAT |
| 7746 | CATCCATCCCATCCAATACAACT | 9010 | AGTTGTATTGGATGGGATGGATG |
| 7747 | CCATCCATCCCATCCAATACAAC | 9011 | GTTGTATTGGATGGGATGGATGG |
| 7748 | TCCATCCATCCCATCCAATACAA | 9012 | TTGTATTGGATGGGATGGATGGA |
| 7749 | TTCCATCCATCCCATCCAATACA | 9013 | TGTATTGGATGGGATGGATGGAA |
| 7750 | CTTCCATCCATCCCATCCAATAC | 9014 | GTATTGGATGGGATGGATGGAAG |
| 7751 | ACTTCCATCCATCCCATCCAATA | 9015 | TATTGGATGGGATGGATGGAAGT |
| 7752 | CACTTCCATCCATCCCATCCAAT | 9016 | ATTGGATGGGATGGATGGAAGTG |
| 7753 | ACACTTCCATCCATCCCATCCAA | 9017 | TTGGATGGGATGGATGGAAGTGT |
| 7754 | CACACTTCCATCCATCCCATCCA | 9018 | TGGATGGGATGGATGGAAGTGTG |
| 7755 | CCACACTTCCATCCATCCCATCC | 9019 | GGATGGGATGGATGGAAGTGTGG |
| 7756 | ACCACACTTCCATCCATCCCATC | 9020 | GATGGGATGGATGGAAGTGTGGT |
| 7757 | AACCACACTTCCATCCATCCCAT | 9021 | ATGGGATGGATGGAAGTGTGGTT |
| 7758 | CAACCACACTTCCATCCATCCCA | 9022 | TGGGATGGATGGAAGTGTGGTTG |
| 7759 | TCAACCACACTTCCATCCATCCC | 9023 | GGGATGGATGGAAGTGTGGTTGA |
| 7760 | CTCAACCACACTTCCATCCATCC | 9024 | GGATGGATGGAAGTGTGGTTGAG |
| 7761 | ACTCAACCACACTTCCATCCATC | 9025 | GATGGATGGAAGTGTGGTTGAGT |
| 7762 | AACTCAACCACACTTCCATCCAT | 9026 | ATGGATGGAAGTGTGGTTGAGTT |
| 7763 | TAACTCAACCACACTTCCATCCA | 9027 | TGGATGGAAGTGTGGTTGAGTTA |
| 7764 | ATAACTCAACCACACTTCCATCC | 9028 | GGATGGAAGTGTGGTTGAGTTAT |
| 7765 | AATAACTCAACCACACTTCCATC | 9029 | GATGGAAGTGTGGTTGAGTTATT |
| 7766 | TAATAACTCAACCACACTTCCAT | 9030 | ATGGAAGTGTGGTTGAGTTATTA |
| 7767 | CTAATAACTCAACCACACTTCCA | 9031 | TGGAAGTGTGGTTGAGTTATTAG |
| 7768 | TCTAATAACTCAACCACACTTCC | 9032 | GGAAGTGTGGTTGAGTTATTAGA |
| 7769 | TTCTAATAACTCAACCACACTTC | 9033 | GAAGTGTGGTTGAGTTATTAGAA |
| 7770 | CTTCTAATAACTCAACCACACTT | 9034 | AAGTGTGGTTGAGTTATTAGAAG |
| 7771 | CCTTCTAATAACTCAACCACACT | 9035 | AGTGTGGTTGAGTTATTAGAAGG |
| 7772 | TCCTTCTAATAACTCAACCACAC | 9036 | GTGTGGTTGAGTTATTAGAAGGA |
| 7773 | TTCCTTCTAATAACTCAACCACA | 9037 | TGTGGTTGAGTTATTAGAAGGAA |
| 7774 | CTTCCTTCTAATAACTCAACCAC | 9038 | GTGGTTGAGTTATTAGAAGGAAG |
| 7775 | TCTTCCTTCTAATAACTCAACCA | 9039 | TGGTTGAGTTATTAGAAGGAAGA |
| 7776 | ATCTTCCTTCTAATAACTCAACC | 9040 | GGTTGAGTTATTAGAAGGAAGAT |
| 7777 | AATCTTCCTTCTAATAACTCAAC | 9041 | GTTGAGTTATTAGAAGGAAGATT |
| 7778 | CAATCTTCCTTCTAATAACTCAA | 9042 | TTGAGTTATTAGAAGGAAGATTG |
| 7779 | TCAATCTTCCTTCTAATAACTCA | 9043 | TGAGTTATTAGAAGGAAGATTGA |
| 7780 | CTCAATCTTCCTTCTAATAACTC | 9044 | GAGTTATTAGAAGGAAGATTGAG |
| 7781 | ACTCAATCTTCCTTCTAATAACT | 9045 | AGTTATTAGAAGGAAGATTGAGT |
| 7782 | TACTCAATCTTCCTTCTAATAAC | 9046 | GTTATTAGAAGGAAGATTGAGTA |
| 7783 | CTACTCAATCTTCCTTCTAATAA | 9047 | TTATTAGAAGGAAGATTGAGTAG |
| 7784 | TCTACTCAATCTTCCTTCTAATA | 9048 | TATTAGAAGGAAGATTGAGTAGA |
| 7785 | ATCTACTCAATCTTCCTTCTAAT | 9049 | ATTAGAAGGAAGATTGAGTAGAT |
| 7786 | TATCTACTCAATCTTCCTTCTAA | 9050 | TTAGAAGGAAGATTGAGTAGATA |
| 7787 | CTATCTACTCAATCTTCCTTCTA | 9051 | TAGAAGGAAGATTGAGTAGATAG |
| 7788 | CCTATCTACTCAATCTTCCTTCT | 9052 | AGAAGGAAGATTGAGTAGATAGG |
| 7789 | ACCTATCTACTCAATCTTCCTTC | 9053 | GAAGGAAGATTGAGTAGATAGGT |
| 7790 | CACCTATCTACTCAATCTTCCTT | 9054 | AAGGAAGATTGAGTAGATAGGTG |
| 7791 | TCACCTATCTACTCAATCTTCCT | 9055 | AGGAAGATTGAGTAGATAGGTGA |
| 7792 | TTCACCTATCTACTCAATCTTCC | 9056 | GGAAGATTGAGTAGATAGGTGAA |
| 7793 | ATTCACCTATCTACTCAATCTTC | 9057 | GAAGATTGAGTAGATAGGTGAAT |
| 7794 | AATTCACCTATCTACTCAATCTT | 9058 | AAGATTGAGTAGATAGGTGAATT |
| 7795 | AAATTCACCTATCTACTCAATCT | 9059 | AGATTGAGTAGATAGGTGAATTT |
| 7796 | CAAATTCACCTATCTACTCAATC | 9060 | GATTGAGTAGATAGGTGAATTTG |
| 7797 | ACAAATTCACCTATCTACTCAAT | 9061 | ATTGAGTAGATAGGTGAATTTGT |
| 7798 | AACAAATTCACCTATCTACTCAA | 9062 | TTGAGTAGATAGGTGAATTTGTT |
| 7799 | CAACAAATTCACCTATCTACTCA | 9063 | TGAGTAGATAGGTGAATTTGTTG |
| 7800 | TCAACAAATTCACCTATCTACTC | 9064 | GAGTAGATAGGTGAATTTGTTGA |
| 7801 | ATCAACAAATTCACCTATCTACT | 9065 | AGTAGATAGGTGAATTTGTTGAT |
| 7802 | TATCAACAAATTCACCTATCTAC | 9066 | GTAGATAGGTGAATTTGTTGATA |
| 7803 | CTATCAACAAATTCACCTATCTA | 9067 | TAGATAGGTGAATTTGTTGATAG |
| 7804 | ACTATCAACAAATTCACCTATCT | 9068 | AGATAGGTGAATTTGTTGATAGT |
| 7805 | GACTATCAACAAATTCACCTATC | 9069 | GATAGGTGAATTTGTTGATAGTC |
| 7806 | TGACTATCAACAAATTCACCTAT | 9070 | ATAGGTGAATTTGTTGATAGTCA |
| 7807 | CTGACTATCAACAAATTCACCTA | 9071 | TAGGTGAATTTGTTGATAGTCAG |
| 7808 | TCTGACTATCAACAAATTCACCT | 9072 | AGGTGAATTTGTTGATAGTCAGA |
| 7809 | ATCTGACTATCAACAAATTCACC | 9073 | GGTGAATTTGTTGATAGTCAGAT |
| 7810 | CATCTGACTATCAACAAATTCAC | 9074 | GTGAATTTGTTGATAGTCAGATG |
| 7811 | CCATCTGACTATCAACAAATTCA | 9075 | TGAATTTGTTGATAGTCAGATGG |
| 7812 | CCCATCTGACTATCAACAAATTC | 9076 | GAATTTGTTGATAGTCAGATGGG |
| 7813 | ACCCATCTGACTATCAACAAATT | 9077 | AATTTGTTGATAGTCAGATGGGT |
| 7814 | TACCCATCTGACTATCAACAAAT | 9078 | ATTTGTTGATAGTCAGATGGGTA |
| 7815 | CTACCCATCTGACTATCAACAAA | 9079 | TTTGTTGATAGTCAGATGGGTAG |
| 7816 | TCTACCCATCTGACTATCAACAA | 9080 | TTGTTGATAGTCAGATGGGTAGA |
| 7817 | ATCTACCCATCTGACTATCAACA | 9081 | TGTTGATAGTCAGATGGGTAGAT |
| 7818 | TATCTACCCATCTGACTATCAAC | 9082 | GTTGATAGTCAGATGGGTAGATA |
| 7819 | CTATCTACCCATCTGACTATCAA | 9083 | TTGATAGTCAGATGGGTAGATAG |
| 7820 | CCTATCTACCCATCTGACTATCA | 9084 | TGATAGTCAGATGGGTAGATAGG |
| 7821 | ACCTATCTACCCATCTGACTATC | 9085 | GATAGTCAGATGGGTAGATAGGT |
| 7822 | TACCTATCTACCCATCTGACTAT | 9086 | ATAGTCAGATGGGTAGATAGGTA |
| 7823 | CTACCTATCTACCCATCTGACTA | 9087 | TAGTCAGATGGGTAGATAGGTAG |
| 7824 | TCTACCTATCTACCCATCTGACT | 9088 | AGTCAGATGGGTAGATAGGTAGA |
| 7825 | ATCTACCTATCTACCCATCTGAC | 9089 | GTCAGATGGGTAGATAGGTAGAT |
| 7826 | CATCTACCTATCTACCCATCTGA | 9090 | TCAGATGGGTAGATAGGTAGATG |
| 7827 | CCATCTACCTATCTACCCATCTG | 9091 | CAGATGGGTAGATAGGTAGATGG |
| 7828 | TCCATCTACCTATCTACCCATCT | 9092 | AGATGGGTAGATAGGTAGATGGA |
| 7829 | ATCCATCTACCTATCTACCCATC | 9093 | GATGGGTAGATAGGTAGATGGAT |
| 7830 | CATCCATCTACCTATCTACCCAT | 9094 | ATGGGTAGATAGGTAGATGGATG |
| 7831 | CCATCCATCTACCTATCTACCCA | 9095 | TGGGTAGATAGGTAGATGGATGG |
| 7832 | TCCATCCATCTACCTATCTACCC | 9096 | GGGTAGATAGGTAGATGGATGGA |
| 7833 | ATCCATCCATCTACCTATCTACC | 9097 | GGTAGATAGGTAGATGGATGGAT |
| 7834 | CATCCATCCATCTACCTATCTAC | 9098 | GTAGATAGGTAGATGGATGGATG |
| 7835 | CCATCCATCCATCTACCTATCTA | 9099 | TAGATAGGTAGATGGATGGATGG |
| 7836 | TCCATCCATCCATCTACCTATCT | 9100 | AGATAGGTAGATGGATGGATGGA |
| 7837 | ATCCATCCATCCATCTACCTATC | 9101 | GATAGGTAGATGGATGGATGGAT |
| 7838 | CATCCATCCATCCATCTACCTAT | 9102 | ATAGGTAGATGGATGGATGGATG |
| 7839 | CCATCCATCCATCCATCTACCTA | 9103 | TAGGTAGATGGATGGATGGATGG |
| 7840 | TCCATCCATCCATCCATCTACCT | 9104 | AGGTAGATGGATGGATGGATGGA |
| 7841 | ATCCATCCATCCATCCATCTACC | 9105 | GGTAGATGGATGGATGGATGGAT |
| 7842 | CATCCATCCATCCATCCATCTAC | 9106 | GTAGATGGATGGATGGATGGATG |
| 7843 | CCATCCATCCATCCATCCATCTA | 9107 | TAGATGGATGGATGGATGGATGG |
| 7844 | TCCATCCATCCATCCATCCATCT | 9108 | AGATGGATGGATGGATGGATGGA |
| 7845 | ATCCATCCATCCATCCATCCATC | 9109 | GATGGATGGATGGATGGATGGAT |
| 7846 | CATCCATCCATCCATCCATCCAT | 9110 | ATGGATGGATGGATGGATGGATG |
| 7847 | ACATCCATCCATCCATCCATCCA | 9111 | TGGATGGATGGATGGATGGATGT |
| 7848 | TACATCCATCCATCCATCCATCC | 9112 | GGATGGATGGATGGATGGATGTA |
| 7849 | ATACATCCATCCATCCATCCATC | 9113 | GATGGATGGATGGATGGATGTAT |
| 7850 | TATACATCCATCCATCCATCCAT | 9114 | ATGGATGGATGGATGGATGTATA |
| 7851 | CTATACATCCATCCATCCATCCA | 9115 | TGGATGGATGGATGGATGTATAG |
| 7852 | CCTATACATCCATCCATCCATCC | 9116 | GGATGGATGGATGGATGTATAGG |
| 7853 | GCCTATACATCCATCCATCCATC | 9117 | GATGGATGGATGGATGTATAGGC |
| 7854 | TGCCTATACATCCATCCATCCAT | 9118 | ATGGATGGATGGATGTATAGGCA |
| 7855 | CTGCCTATACATCCATCCATCCA | 9119 | TGGATGGATGGATGTATAGGCAG |
| 7856 | TCTGCCTATACATCCATCCATCC | 9120 | GGATGGATGGATGTATAGGCAGA |
| 7857 | ATCTGCCTATACATCCATCCATC | 9121 | GATGGATGGATGTATAGGCAGAT |
| 7858 | CATCTGCCTATACATCCATCCAT | 9122 | ATGGATGGATGTATAGGCAGATG |
| 7859 | CCATCTGCCTATACATCCATCCA | 9123 | TGGATGGATGTATAGGCAGATGG |
| 7860 | TCCATCTGCCTATACATCCATCC | 9124 | GGATGGATGTATAGGCAGATGGA |
| 7861 | GTCCATCTGCCTATACATCCATC | 9125 | GATGGATGTATAGGCAGATGGAC |
| 7862 | TGTCCATCTGCCTATACATCCAT | 9126 | ATGGATGTATAGGCAGATGGACA |
| 7863 | TTGTCCATCTGCCTATACATCCA | 9127 | TGGATGTATAGGCAGATGGACAA |
| 7864 | TTTGTCCATCTGCCTATACATCC | 9128 | GGATGTATAGGCAGATGGACAAA |
| 7865 | ATTTGTCCATCTGCCTATACATC | 9129 | GATGTATAGGCAGATGGACAAAT |
| 7866 | CATTTGTCCATCTGCCTATACAT | 9130 | ATGTATAGGCAGATGGACAAATG |
| 7867 | CCATTTGTCCATCTGCCTATACA | 9131 | TGTATAGGCAGATGGACAAATGG |
| 7868 | TCCATTTGTCCATCTGCCTATAC | 9132 | GTATAGGCAGATGGACAAATGGA |
| 7869 | ATCCATTTGTCCATCTGCCTATA | 9133 | TATAGGCAGATGGACAAATGGAT |
| 7870 | CATCCATTTGTCCATCTGCCTAT | 9134 | ATAGGCAGATGGACAAATGGATG |
| 7871 | TCATCCATTTGTCCATCTGCCTA | 9135 | TAGGCAGATGGACAAATGGATGA |
| 7872 | TTCATCCATTTGTCCATCTGCCT | 9136 | AGGCAGATGGACAAATGGATGAA |
| 7873 | ATTCATCCATTTGTCCATCTGCC | 9137 | GGCAGATGGACAAATGGATGAAT |
| 7874 | CATTCATCCATTTGTCCATCTGC | 9138 | GCAGATGGACAAATGGATGAATG |
| 7875 | CCATTCATCCATTTGTCCATCTG | 9139 | CAGATGGACAAATGGATGAATGG |
| 7876 | CCCATTCATCCATTTGTCCATCT | 9140 | AGATGGACAAATGGATGAATGGG |
| 7877 | ACCCATTCATCCATTTGTCCATC | 9141 | GATGGACAAATGGATGAATGGGT |
| 7878 | CACCCATTCATCCATTTGTCCAT | 9142 | ATGGACAAATGGATGAATGGGTG |
| 7879 | CCACCCATTCATCCATTTGTCCA | 9143 | TGGACAAATGGATGAATGGGTGG |
| 7880 | CCCACCCATTCATCCATTTGTCC | 9144 | GGACAAATGGATGAATGGGTGGG |
| 7881 | ACCCACCCATTCATCCATTTGTC | 9145 | GACAAATGGATGAATGGGTGGGT |
| 7882 | CACCCACCCATTCATCCATTTGT | 9146 | ACAAATGGATGAATGGGTGGGTG |
| 7883 | CCACCCACCCATTCATCCATTTG | 9147 | CAAATGGATGAATGGGTGGGTGG |
| 7884 | TCCACCCACCCATTCATCCATTT | 9148 | AAATGGATGAATGGGTGGGTGGA |
| 7885 | ATCCACCCACCCATTCATCCATT | 9149 | AATGGATGAATGGGTGGGTGGAT |
| 7886 | CATCCACCCACCCATTCATCCAT | 9150 | ATGGATGAATGGGTGGGTGGATG |
| 7887 | TCATCCACCCACCCATTCATCCA | 9151 | TGGATGAATGGGTGGGTGGATGA |
| 7888 | TTCATCCACCCACCCATTCATCC | 9152 | GGATGAATGGGTGGGTGGATGAA |
| 7889 | ATTCATCCACCCACCCATTCATC | 9153 | GATGAATGGGTGGGTGGATGAAT |
| 7890 | CATTCATCCACCCACCCATTCAT | 9154 | ATGAATGGGTGGGTGGATGAATG |
| 7891 | CCATTCATCCACCCACCCATTCA | 9155 | TGAATGGGTGGGTGGATGAATGG |
| 7892 | TCCATTCATCCACCCACCCATTC | 9156 | GAATGGGTGGGTGGATGAATGGA |
| 7893 | TTCCATTCATCCACCCACCCATT | 9157 | AATGGGTGGGTGGATGAATGGAA |
| 7894 | CTTCCATTCATCCACCCACCCAT | 9158 | ATGGGTGGGTGGATGAATGGAAG |
| 7895 | CCTTCCATTCATCCACCCACCCA | 9159 | TGGGTGGGTGGATGAATGGAAGG |
| 7896 | TCCTTCCATTCATCCACCCACCC | 9160 | GGGTGGGTGGATGAATGGAAGGA |
| 7897 | ATCCTTCCATTCATCCACCCACC | 9161 | GGTGGGTGGATGAATGGAAGGAT |
| 7898 | CATCCTTCCATTCATCCACCCAC | 9162 | GTGGGTGGATGAATGGAAGGATG |
| 7899 | ACATCCTTCCATTCATCCACCCA | 9163 | TGGGTGGATGAATGGAAGGATGT |
| 7900 | CACATCCTTCCATTCATCCACCC | 9164 | GGGTGGATGAATGGAAGGATGTG |
| 7901 | ACACATCCTTCCATTCATCCACC | 9165 | GGTGGATGAATGGAAGGATGTGT |
| 7902 | CACACATCCTTCCATTCATCCAC | 9166 | GTGGATGAATGGAAGGATGTGTG |
| 7903 | CCACACATCCTTCCATTCATCCA | 9167 | TGGATGAATGGAAGGATGTGTGG |
| 7904 | ACCACACATCCTTCCATTCATCC | 9168 | GGATGAATGGAAGGATGTGTGGT |
| 7905 | AACCACACATCCTTCCATTCATC | 9169 | GATGAATGGAAGGATGTGTGGTT |
| 7906 | CAACCACACATCCTTCCATTCAT | 9170 | ATGAATGGAAGGATGTGTGGTTG |
| 7907 | TCAACCACACATCCTTCCATTCA | 9171 | TGAATGGAAGGATGTGTGGTTGA |
| 7908 | TTCAACCACACATCCTTCCATTC | 9172 | GAATGGAAGGATGTGTGGTTGAA |
| 7909 | GTTCAACCACACATCCTTCCATT | 9173 | AATGGAAGGATGTGTGGTTGAAC |
| 7910 | AGTTCAACCACACATCCTTCCAT | 9174 | ATGGAAGGATGTGTGGTTGAACT |
| 7911 | TAGTTCAACCACACATCCTTCCA | 9175 | TGGAAGGATGTGTGGTTGAACTA |
| 7912 | ATAGTTCAACCACACATCCTTCC | 9176 | GGAAGGATGTGTGGTTGAACTAT |
| 7913 | AATAGTTCAACCACACATCCTTC | 9177 | GAAGGATGTGTGGTTGAACTATT |
| 7914 | CAATAGTTCAACCACACATCCTT | 9178 | AAGGATGTGTGGTTGAACTATTG |
| 7915 | GCAATAGTTCAACCACACATCCT | 9179 | AGGATGTGTGGTTGAACTATTGC |
| 7916 | TGCAATAGTTCAACCACACATCC | 9180 | GGATGTGTGGTTGAACTATTGCA |
| 7917 | TTGCAATAGTTCAACCACACATC | 9181 | GATGTGTGGTTGAACTATTGCAA |
| 7918 | CTTGCAATAGTTCAACCACACAT | 9182 | ATGTGTGGTTGAACTATTGCAAG |
| 7919 | ACTTGCAATAGTTCAACCACACA | 9183 | TGTGTGGTTGAACTATTGCAAGT |
| 7920 | TACTTGCAATAGTTCAACCACAC | 9184 | GTGTGGTTGAACTATTGCAAGTA |
| 7921 | ATACTTGCAATAGTTCAACCACA | 9185 | TGTGGTTGAACTATTGCAAGTAT |
| 7922 | AATACTTGCAATAGTTCAACCAC | 9186 | GTGGTTGAACTATTGCAAGTATT |
| 7923 | CAATACTTGCAATAGTTCAACCA | 9187 | TGGTTGAACTATTGCAAGTATTG |
| 7924 | TCAATACTTGCAATAGTTCAACC | 9188 | GGTTGAACTATTGCAAGTATTGA |
| 7925 | ATCAATACTTGCAATAGTTCAAC | 9189 | GTTGAACTATTGCAAGTATTGAT |
| 7926 | TATCAATACTTGCAATAGTTCAA | 9190 | TTGAACTATTGCAAGTATTGATA |
| 7927 | TTATCAATACTTGCAATAGTTCA | 9191 | TGAACTATTGCAAGTATTGATAA |
| 7928 | ATTATCAATACTTGCAATAGTTC | 9192 | GAACTATTGCAAGTATTGATAAT |
| 7929 | AATTATCAATACTTGCAATAGTT | 9193 | AACTATTGCAAGTATTGATAATT |
| 7930 | CAATTATCAATACTTGCAATAGT | 9194 | ACTATTGCAAGTATTGATAATTG |
| 7931 | CCAATTATCAATACTTGCAATAG | 9195 | CTATTGCAAGTATTGATAATTGG |
| 7932 | CCCAATTATCAATACTTGCAATA | 9196 | TATTGCAAGTATTGATAATTGGG |
| 7933 | ACCCAATTATCAATACTTGCAAT | 9197 | ATTGCAAGTATTGATAATTGGGT |
| 7934 | AACCCAATTATCAATACTTGCAA | 9198 | TTGCAAGTATTGATAATTGGGTT |
| 7935 | GAACCCAATTATCAATACTTGCA | 9199 | TGCAAGTATTGATAATTGGGTTC |
| 7936 | TGAACCCAATTATCAATACTTGC | 9200 | GCAAGTATTGATAATTGGGTTCA |
| 7937 | ATGAACCCAATTATCAATACTTG | 9201 | CAAGTATTGATAATTGGGTTCAT |
| 7938 | TATGAACCCAATTATCAATACTT | 9202 | AAGTATTGATAATTGGGTTCATA |
| 7939 | TTATGAACCCAATTATCAATACT | 9203 | AGTATTGATAATTGGGTTCATAA |
| 7940 | ATTATGAACCCAATTATCAATAC | 9204 | GTATTGATAATTGGGTTCATAAT |
| 7941 | AATTATGAACCCAATTATCAATA | 9205 | TATTGATAATTGGGTTCATAATT |
| 7942 | AAATTATGAACCCAATTATCAAT | 9206 | ATTGATAATTGGGTTCATAATTT |
| 7943 | GAAATTATGAACCCAATTATCAA | 9207 | TTGATAATTGGGTTCATAATTTC |
| 7944 | AGAAATTATGAACCCAATTATCA | 9208 | TGATAATTGGGTTCATAATTTCT |
| 7945 | CAGAAATTATGAACCCAATTATC | 9209 | GATAATTGGGTTCATAATTTCTG |
| 7946 | TCAGAAATTATGAACCCAATTAT | 9210 | ATAATTGGGTTCATAATTTCTGA |
| 7947 | TTCAGAAATTATGAACCCAATTA | 9211 | TAATTGGGTTCATAATTTCTGAA |
| 7948 | ATTCAGAAATTATGAACCCAATT | 9212 | AATTGGGTTCATAATTTCTGAAT |
| 7949 | TATTCAGAAATTATGAACCCAAT | 9213 | ATTGGGTTCATAATTTCTGAATA |
| 7950 | ATATTCAGAAATTATGAACCCAA | 9214 | TTGGGTTCATAATTTCTGAATAT |
| 7951 | AATATTCAGAAATTATGAACCCA | 9215 | TGGGTTCATAATTTCTGAATATT |
| 7952 | AAATATTCAGAAATTATGAACCC | 9216 | GGGTTCATAATTTCTGAATATTT |
| 7953 | TAAATATTCAGAAATTATGAACC | 9217 | GGTTCATAATTTCTGAATATTTA |
| 7954 | CTAAATATTCAGAAATTATGAAC | 9218 | GTTCATAATTTCTGAATATTTAG |
| 7955 | TCTAAATATTCAGAAATTATGAA | 9219 | TTCATAATTTCTGAATATTTAGA |
| 7956 | ATCTAAATATTCAGAAATTATGA | 9220 | TCATAATTTCTGAATATTTAGAT |
| 7957 | CATCTAAATATTCAGAAATTATG | 9221 | CATAATTTCTGAATATTTAGATG |
| 7958 | CCATCTAAATATTCAGAAATTAT | 9222 | ATAATTTCTGAATATTTAGATGG |
| 7959 | TCCATCTAAATATTCAGAAATTA | 9223 | TAATTTCTGAATATTTAGATGGA |
| 7960 | ATCCATCTAAATATTCAGAAATT | 9224 | AATTTCTGAATATTTAGATGGAT |
| 7961 | CATCCATCTAAATATTCAGAAAT | 9225 | ATTTCTGAATATTTAGATGGATG |
| 7962 | CCATCCATCTAAATATTCAGAAA | 9226 | TTTCTGAATATTTAGATGGATGG |
| 7963 | ACCATCCATCTAAATATTCAGAA | 9227 | TTCTGAATATTTAGATGGATGGT |
| 7964 | AACCATCCATCTAAATATTCAGA | 9228 | TCTGAATATTTAGATGGATGGTT |
| 7965 | CAACCATCCATCTAAATATTCAG | 9229 | CTGAATATTTAGATGGATGGTTG |
| 7966 | ACAACCATCCATCTAAATATTCA | 9230 | TGAATATTTAGATGGATGGTTGT |
| 7967 | CACAACCATCCATCTAAATATTC | 9231 | GAATATTTAGATGGATGGTTGTG |
| 7968 | TCACAACCATCCATCTAAATATT | 9232 | AATATTTAGATGGATGGTTGTGA |
| 7969 | CTCACAACCATCCATCTAAATAT | 9233 | ATATTTAGATGGATGGTTGTGAG |
| 7970 | ACTCACAACCATCCATCTAAATA | 9234 | TATTTAGATGGATGGTTGTGAGT |
| 7971 | CACTCACAACCATCCATCTAAAT | 9235 | ATTTAGATGGATGGTTGTGAGTG |
| 7972 | CCACTCACAACCATCCATCTAAA | 9236 | TTTAGATGGATGGTTGTGAGTGG |
| 7973 | GCCACTCACAACCATCCATCTAA | 9237 | TTAGATGGATGGTTGTGAGTGGC |
| 7974 | AGCCACTCACAACCATCCATCTA | 9238 | TAGATGGATGGTTGTGAGTGGCT |
| 7975 | CAGCCACTCACAACCATCCATCT | 9239 | AGATGGATGGTTGTGAGTGGCTG |
| 7976 | CCAGCCACTCACAACCATCCATC | 9240 | GATGGATGGTTGTGAGTGGCTGG |
| 7977 | ACCAGCCACTCACAACCATCCAT | 9241 | ATGGATGGTTGTGAGTGGCTGGT |
| 7978 | CACCAGCCACTCACAACCATCCA | 9242 | TGGATGGTTGTGAGTGGCTGGTG |
| 7979 | CCACCAGCCACTCACAACCATCC | 9243 | GGATGGTTGTGAGTGGCTGGTGG |
| 7980 | TCCACCAGCCACTCACAACCATC | 9244 | GATGGTTGTGAGTGGCTGGTGGA |
| 7981 | GTCCACCAGCCACTCACAACCAT | 9245 | ATGGTTGTGAGTGGCTGGTGGAC |
| 7982 | TGTCCACCAGCCACTCACAACCA | 9246 | TGGTTGTGAGTGGCTGGTGGACA |
| 7983 | CTGTCCACCAGCCACTCACAACC | 9247 | GGTTGTGAGTGGCTGGTGGACAG |
| 7984 | TCTGTCCACCAGCCACTCACAAC | 9248 | GTTGTGAGTGGCTGGTGGACAGA |
| 7985 | GTCTGTCCACCAGCCACTCACAA | 9249 | TTGTGAGTGGCTGGTGGACAGAC |
| 7986 | CGTCTGTCCACCAGCCACTCACA | 9250 | TGTGAGTGGCTGGTGGACAGACG |
| 7987 | TCGTCTGTCCACCAGCCACTCAC | 9251 | GTGAGTGGCTGGTGGACAGACGA |
| 7988 | TTCGTCTGTCCACCAGCCACTCA | 9252 | TGAGTGGCTGGTGGACAGACGAA |
| 7989 | TTTCGTCTGTCCACCAGCCACTC | 9253 | GAGTGGCTGGTGGACAGACGAAA |
| 7990 | TTTTCGTCTGTCCACCAGCCACT | 9254 | AGTGGCTGGTGGACAGACGAAAA |
| 7991 | TTTTTCGTCTGTCCACCAGCCAC | 9255 | GTGGCTGGTGGACAGACGAAAAA |
| 7992 | ATTTTTCGTCTGTCCACCAGCCA | 9256 | TGGCTGGTGGACAGACGAAAAAT |
| 7993 | CATTTTTCGTCTGTCCACCAGCC | 9257 | GGCTGGTGGACAGACGAAAAATG |
| 7994 | CCATTTTTCGTCTGTCCACCAGC | 9258 | GCTGGTGGACAGACGAAAAATGG |
| 7995 | TCCATTTTTCGTCTGTCCACCAG | 9259 | CTGGTGGACAGACGAAAAATGGA |
| 7996 | ATCCATTTTTCGTCTGTCCACCA | 9260 | TGGTGGACAGACGAAAAATGGAT |
| 7997 | CATCCATTTTTCGTCTGTCCACC | 9261 | GGTGGACAGACGAAAAATGGATG |
| 7998 | CCATCCATTTTTCGTCTGTCCAC | 9262 | GTGGACAGACGAAAAATGGATGG |
| 7999 | ACCATCCATTTTTCGTCTGTCCA | 9263 | TGGACAGACGAAAAATGGATGGT |
| 8000 | AACCATCCATTTTTCGTCTGTCC | 9264 | GGACAGACGAAAAATGGATGGTT |
| 8001 | CAACCATCCATTTTTCGTCTGTC | 9265 | GACAGACGAAAAATGGATGGTTG |
| 8002 | CCAACCATCCATTTTTCGTCTGT | 9266 | ACAGACGAAAAATGGATGGTTGG |
| 8003 | TCCAACCATCCATTTTTCGTCTG | 9267 | CAGACGAAAAATGGATGGTTGGA |
| 8004 | ATCCAACCATCCATTTTTCGTCT | 9268 | AGACGAAAAATGGATGGTTGGAT |
| 8005 | TATCCAACCATCCATTTTTCGTC | 9269 | GACGAAAAATGGATGGTTGGATA |
| 8006 | TTATCCAACCATCCATTTTTCGT | 9270 | ACGAAAAATGGATGGTTGGATAA |
| 8007 | TTTATCCAACCATCCATTTTTCG | 9271 | CGAAAAATGGATGGTTGGATAAA |
| 8008 | ATTTATCCAACCATCCATTTTTC | 9272 | GAAAAATGGATGGTTGGATAAAT |
| 8009 | AATTTATCCAACCATCCATTTTT | 9273 | AAAAATGGATGGTTGGATAAATT |
| 8010 | CAATTTATCCAACCATCCATTTT | 9274 | AAAATGGATGGTTGGATAAATTG |
| 8011 | TCAATTTATCCAACCATCCATTT | 9275 | AAATGGATGGTTGGATAAATTGA |
| 8012 | ATCAATTTATCCAACCATCCATT | 9276 | AATGGATGGTTGGATAAATTGAT |
| 8013 | CATCAATTTATCCAACCATCCAT | 9277 | ATGGATGGTTGGATAAATTGATG |
| 8014 | CCATCAATTTATCCAACCATCCA | 9278 | TGGATGGTTGGATAAATTGATGG |
| 8015 | CCCATCAATTTATCCAACCATCC | 9279 | GGATGGTTGGATAAATTGATGGG |
| 8016 | ACCCATCAATTTATCCAACCATC | 9280 | GATGGTTGGATAAATTGATGGGT |
| 8017 | CACCCATCAATTTATCCAACCAT | 9281 | ATGGTTGGATAAATTGATGGGTG |
| 8018 | CCACCCATCAATTTATCCAACCA | 9282 | TGGTTGGATAAATTGATGGGTGG |
| 8019 | TCCACCCATCAATTTATCCAACC | 9283 | GGTTGGATAAATTGATGGGTGGA |
| 8020 | ATCCACCCATCAATTTATCCAAC | 9284 | GTTGGATAAATTGATGGGTGGAT |
| 8021 | CATCCACCCATCAATTTATCCAA | 9285 | TTGGATAAATTGATGGGTGGATG |
| 8022 | CCATCCACCCATCAATTTATCCA | 9286 | TGGATAAATTGATGGGTGGATGG |
| 8023 | TCCATCCACCCATCAATTTATCC | 9287 | GGATAAATTGATGGGTGGATGGA |
| 8024 | ATCCATCCACCCATCAATTTATC | 9288 | GATAAATTGATGGGTGGATGGAT |
| 8025 | CATCCATCCACCCATCAATTTAT | 9289 | ATAAATTGATGGGTGGATGGATG |
| 8026 | CCATCCATCCACCCATCAATTTA | 9290 | TAAATTGATGGGTGGATGGATGG |
| 8027 | ACCATCCATCCACCCATCAATTT | 9291 | AAATTGATGGGTGGATGGATGGT |
| 8028 | AACCATCCATCCACCCATCAATT | 9292 | AATTGATGGGTGGATGGATGGTT |
| 8029 | CAACCATCCATCCACCCATCAAT | 9293 | ATTGATGGGTGGATGGATGGTTG |
| 8030 | CCAACCATCCATCCACCCATCAA | 9294 | TTGATGGGTGGATGGATGGTTGG |
| 8031 | ACCAACCATCCATCCACCCATCA | 9295 | TGATGGGTGGATGGATGGTTGGT |
| 8032 | AACCAACCATCCATCCACCCATC | 9296 | GATGGGTGGATGGATGGTTGGTT |
| 8033 | CAACCAACCATCCATCCACCCAT | 9297 | ATGGGTGGATGGATGGTTGGTTG |
| 8034 | ACAACCAACCATCCATCCACCCA | 9298 | TGGGTGGATGGATGGTTGGTTGT |
| 8035 | TACAACCAACCATCCATCCACCC | 9299 | GGGTGGATGGATGGTTGGTTGTA |
| 8036 | ATACAACCAACCATCCATCCACC | 9300 | GGTGGATGGATGGTTGGTTGTAT |
| 8037 | CATACAACCAACCATCCATCCAC | 9301 | GTGGATGGATGGTTGGTTGTATG |
| 8038 | TCATACAACCAACCATCCATCCA | 9302 | TGGATGGATGGTTGGTTGTATGA |
| 8039 | TTCATACAACCAACCATCCATCC | 9303 | GGATGGATGGTTGGTTGTATGAA |
| 8040 | TTTCATACAACCAACCATCCATC | 9304 | GATGGATGGTTGGTTGTATGAAA |
| 8041 | CTTTCATACAACCAACCATCCAT | 9305 | ATGGATGGTTGGTTGTATGAAAG |
| 8042 | TCTTTCATACAACCAACCATCCA | 9306 | TGGATGGTTGGTTGTATGAAAGA |
| 8043 | TTCTTTCATACAACCAACCATCC | 9307 | GGATGGTTGGTTGTATGAAAGAA |
| 8044 | ATTCTTTCATACAACCAACCATC | 9308 | GATGGTTGGTTGTATGAAAGAAT |
| 8045 | CATTCTTTCATACAACCAACCAT | 9309 | ATGGTTGGTTGTATGAAAGAATG |
| 8046 | TCATTCTTTCATACAACCAACCA | 9310 | TGGTTGGTTGTATGAAAGAATGA |
| 8047 | TTCATTCTTTCATACAACCAACC | 9311 | GGTTGGTTGTATGAAAGAATGAA |
| 8048 | ATTCATTCTTTCATACAACCAAC | 9312 | GTTGGTTGTATGAAAGAATGAAT |
| 8049 | CATTCATTCTTTCATACAACCAA | 9313 | TTGGTTGTATGAAAGAATGAATG |
| 8050 | TCATTCATTCTTTCATACAACCA | 9314 | TGGTTGTATGAAAGAATGAATGA |
| 8051 | ATCATTCATTCTTTCATACAACC | 9315 | GGTTGTATGAAAGAATGAATGAT |
| 8052 | AATCATTCATTCTTTCATACAAC | 9316 | GTTGTATGAAAGAATGAATGATT |
| 8053 | CAATCATTCATTCTTTCATACAA | 9317 | TTGTATGAAAGAATGAATGATTG |
| 8054 | CCAATCATTCATTCTTTCATACA | 9318 | TGTATGAAAGAATGAATGATTGG |
| 8055 | CCCAATCATTCATTCTTTCATAC | 9319 | GTATGAAAGAATGAATGATTGGG |
| 8056 | ACCCAATCATTCATTCTTTCATA | 9320 | TATGAAAGAATGAATGATTGGGT |
| 8057 | TACCCAATCATTCATTCTTTCAT | 9321 | ATGAAAGAATGAATGATTGGGTA |
| 8058 | CTACCCAATCATTCATTCTTTCA | 9322 | TGAAAGAATGAATGATTGGGTAG |
| 8059 | CCTACCCAATCATTCATTCTTTC | 9323 | GAAAGAATGAATGATTGGGTAGG |
| 8060 | ACCTACCCAATCATTCATTCTTT | 9324 | AAAGAATGAATGATTGGGTAGGT |
| 8061 | CACCTACCCAATCATTCATTCTT | 9325 | AAGAATGAATGATTGGGTAGGTG |
| 8062 | CCACCTACCCAATCATTCATTCT | 9326 | AGAATGAATGATTGGGTAGGTGG |
| 8063 | TCCACCTACCCAATCATTCATTC | 9327 | GAATGAATGATTGGGTAGGTGGA |
| 8064 | ATCCACCTACCCAATCATTCATT | 9328 | AATGAATGATTGGGTAGGTGGAT |
| 8065 | AATCCACCTACCCAATCATTCAT | 9329 | ATGAATGATTGGGTAGGTGGATT |
| 8066 | TAATCCACCTACCCAATCATTCA | 9330 | TGAATGATTGGGTAGGTGGATTA |
| 8067 | TTAATCCACCTACCCAATCATTC | 9331 | GAATGATTGGGTAGGTGGATTAA |
| 8068 | CTTAATCCACCTACCCAATCATT | 9332 | AATGATTGGGTAGGTGGATTAAG |
| 8069 | ACTTAATCCACCTACCCAATCAT | 9333 | ATGATTGGGTAGGTGGATTAAGT |
| 8070 | AACTTAATCCACCTACCCAATCA | 9334 | TGATTGGGTAGGTGGATTAAGTT |
| 8071 | CAACTTAATCCACCTACCCAATC | 9335 | GATTGGGTAGGTGGATTAAGTTG |
| 8072 | GCAACTTAATCCACCTACCCAAT | 9336 | ATTGGGTAGGTGGATTAAGTTGC |
| 8073 | CGCAACTTAATCCACCTACCCAA | 9337 | TTGGGTAGGTGGATTAAGTTGCG |
| 8074 | CCGCAACTTAATCCACCTACCCA | 9338 | TGGGTAGGTGGATTAAGTTGCGG |
| 8075 | TCCGCAACTTAATCCACCTACCC | 9339 | GGGTAGGTGGATTAAGTTGCGGA |
| 8076 | ATCCGCAACTTAATCCACCTACC | 9340 | GGTAGGTGGATTAAGTTGCGGAT |
| 8077 | GATCCGCAACTTAATCCACCTAC | 9341 | GTAGGTGGATTAAGTTGCGGATC |
| 8078 | TGATCCGCAACTTAATCCACCTA | 9342 | TAGGTGGATTAAGTTGCGGATCA |
| 8079 | TTGATCCGCAACTTAATCCACCT | 9343 | AGGTGGATTAAGTTGCGGATCAA |
| 8080 | ATTGATCCGCAACTTAATCCACC | 9344 | GGTGGATTAAGTTGCGGATCAAT |
| 8081 | CATTGATCCGCAACTTAATCCAC | 9345 | GTGGATTAAGTTGCGGATCAATG |
| 8082 | ACATTGATCCGCAACTTAATCCA | 9346 | TGGATTAAGTTGCGGATCAATGT |
| 8083 | TACATTGATCCGCAACTTAATCC | 9347 | GGATTAAGTTGCGGATCAATGTA |
| 8084 | ATACATTGATCCGCAACTTAATC | 9348 | GATTAAGTTGCGGATCAATGTAT |
| 8085 | CATACATTGATCCGCAACTTAAT | 9349 | ATTAAGTTGCGGATCAATGTATG |
| 8086 | CCATACATTGATCCGCAACTTAA | 9350 | TTAAGTTGCGGATCAATGTATGG |
| 8087 | CCCATACATTGATCCGCAACTTA | 9351 | TAAGTTGCGGATCAATGTATGGG |
| 8088 | TCCCATACATTGATCCGCAACTT | 9352 | AAGTTGCGGATCAATGTATGGGA |
| 8089 | ATCCCATACATTGATCCGCAACT | 9353 | AGTTGCGGATCAATGTATGGGAT |
| 8090 | CATCCCATACATTGATCCGCAAC | 9354 | GTTGCGGATCAATGTATGGGATG |
| 8091 | CCATCCCATACATTGATCCGCAA | 9355 | TTGCGGATCAATGTATGGGATGG |
| 8092 | TCCATCCCATACATTGATCCGCA | 9356 | TGCGGATCAATGTATGGGATGGA |
| 8093 | ATCCATCCCATACATTGATCCGC | 9357 | GCGGATCAATGTATGGGATGGAT |
| 8094 | CATCCATCCCATACATTGATCCG | 9358 | CGGATCAATGTATGGGATGGATG |
| 8095 | TCATCCATCCCATACATTGATCC | 9359 | GGATCAATGTATGGGATGGATGA |
| 8096 | TTCATCCATCCCATACATTGATC | 9360 | GATCAATGTATGGGATGGATGAA |
| 8097 | ATTCATCCATCCCATACATTGAT | 9361 | ATCAATGTATGGGATGGATGAAT |
| 8098 | CATTCATCCATCCCATACATTGA | 9362 | TCAATGTATGGGATGGATGAATG |
| 8099 | CCATTCATCCATCCCATACATTG | 9363 | CAATGTATGGGATGGATGAATGG |
| 8100 | TCCATTCATCCATCCCATACATT | 9364 | AATGTATGGGATGGATGAATGGA |
| 8101 | ATCCATTCATCCATCCCATACAT | 9365 | ATGTATGGGATGGATGAATGGAT |
| 8102 | CATCCATTCATCCATCCCATACA | 9366 | TGTATGGGATGGATGAATGGATG |
| 8103 | CCATCCATTCATCCATCCCATAC | 9367 | GTATGGGATGGATGAATGGATGG |
| 8104 | TCCATCCATTCATCCATCCCATA | 9368 | TATGGGATGGATGAATGGATGGA |
| 8105 | ATCCATCCATTCATCCATCCCAT | 9369 | ATGGGATGGATGAATGGATGGAT |
| 8106 | CATCCATCCATTCATCCATCCCA | 9370 | TGGGATGGATGAATGGATGGATG |
| 8107 | CCATCCATCCATTCATCCATCCC | 9371 | GGGATGGATGAATGGATGGATGG |
| 8108 | TCCATCCATCCATTCATCCATCC | 9372 | GGATGGATGAATGGATGGATGGA |
| 8109 | ATCCATCCATCCATTCATCCATC | 9373 | GATGGATGAATGGATGGATGGAT |
| 8110 | CATCCATCCATCCATTCATCCAT | 9374 | ATGGATGAATGGATGGATGGATG |
| 8111 | CCATCCATCCATCCATTCATCCA | 9375 | TGGATGAATGGATGGATGGATGG |
| 8112 | TCCATCCATCCATCCATTCATCC | 9376 | GGATGAATGGATGGATGGATGGA |
| 8113 | ATCCATCCATCCATCCATTCATC | 9377 | GATGAATGGATGGATGGATGGAT |
| 8114 | CATCCATCCATCCATCCATTCAT | 9378 | ATGAATGGATGGATGGATGGATG |
| 8115 | ACATCCATCCATCCATCCATTCA | 9379 | TGAATGGATGGATGGATGGATGT |
| 8116 | CACATCCATCCATCCATCCATTC | 9380 | GAATGGATGGATGGATGGATGTG |
| 8117 | ACACATCCATCCATCCATCCATT | 9381 | AATGGATGGATGGATGGATGTGT |
| 8118 | CACACATCCATCCATCCATCCAT | 9382 | ATGGATGGATGGATGGATGTGTG |
| 8119 | CCACACATCCATCCATCCATCCA | 9383 | TGGATGGATGGATGGATGTGTGG |
| 8120 | ACCACACATCCATCCATCCATCC | 9384 | GGATGGATGGATGGATGTGTGGT |
| 8121 | AACCACACATCCATCCATCCATC | 9385 | GATGGATGGATGGATGTGTGGTT |
| 8122 | CAACCACACATCCATCCATCCAT | 9386 | ATGGATGGATGGATGTGTGGTTG |
| 8123 | TCAACCACACATCCATCCATCCA | 9387 | TGGATGGATGGATGTGTGGTTGA |
| 8124 | TTCAACCACACATCCATCCATCC | 9388 | GGATGGATGGATGTGTGGTTGAA |
| 8125 | ATTCAACCACACATCCATCCATC | 9389 | GATGGATGGATGTGTGGTTGAAT |
| 8126 | AATTCAACCACACATCCATCCAT | 9390 | ATGGATGGATGTGTGGTTGAATT |
| 8127 | TAATTCAACCACACATCCATCCA | 9391 | TGGATGGATGTGTGGTTGAATTA |
| 8128 | GTAATTCAACCACACATCCATCC | 9392 | GGATGGATGTGTGGTTGAATTAC |
| 8129 | AGTAATTCAACCACACATCCATC | 9393 | GATGGATGTGTGGTTGAATTACT |
| 8130 | CAGTAATTCAACCACACATCCAT | 9394 | ATGGATGTGTGGTTGAATTACTG |
| 8131 | TCAGTAATTCAACCACACATCCA | 9395 | TGGATGTGTGGTTGAATTACTGA |
| 8132 | TTCAGTAATTCAACCACACATCC | 9396 | GGATGTGTGGTTGAATTACTGAA |
| 8133 | TTTCAGTAATTCAACCACACATC | 9397 | GATGTGTGGTTGAATTACTGAAA |
| 8134 | CTTTCAGTAATTCAACCACACAT | 9398 | ATGTGTGGTTGAATTACTGAAAG |
| 8135 | CCTTTCAGTAATTCAACCACACA | 9399 | TGTGTGGTTGAATTACTGAAAGG |
| 8136 | ACCTTTCAGTAATTCAACCACAC | 9400 | GTGTGGTTGAATTACTGAAAGGT |
| 8137 | AACCTTTCAGTAATTCAACCACA | 9401 | TGTGGTTGAATTACTGAAAGGTT |
| 8138 | CAACCTTTCAGTAATTCAACCAC | 9402 | GTGGTTGAATTACTGAAAGGTTG |
| 8139 | CCAACCTTTCAGTAATTCAACCA | 9403 | TGGTTGAATTACTGAAAGGTTGG |
| 8140 | TCCAACCTTTCAGTAATTCAACC | 9404 | GGTTGAATTACTGAAAGGTTGGA |
| 8141 | TTCCAACCTTTCAGTAATTCAAC | 9405 | GTTGAATTACTGAAAGGTTGGAA |
| 8142 | CTTCCAACCTTTCAGTAATTCAA | 9406 | TTGAATTACTGAAAGGTTGGAAG |
| 8143 | TCTTCCAACCTTTCAGTAATTCA | 9407 | TGAATTACTGAAAGGTTGGAAGA |
| 8144 | CTCTTCCAACCTTTCAGTAATTC | 9408 | GAATTACTGAAAGGTTGGAAGAG |
| 8145 | ACTCTTCCAACCTTTCAGTAATT | 9409 | AATTACTGAAAGGTTGGAAGAGT |
| 8146 | CACTCTTCCAACCTTTCAGTAAT | 9410 | ATTACTGAAAGGTTGGAAGAGTG |
| 8147 | CCACTCTTCCAACCTTTCAGTAA | 9411 | TTACTGAAAGGTTGGAAGAGTGG |
| 8148 | TCCACTCTTCCAACCTTTCAGTA | 9412 | TACTGAAAGGTTGGAAGAGTGGA |
| 8149 | ATCCACTCTTCCAACCTTTCAGT | 9413 | ACTGAAAGGTTGGAAGAGTGGAT |
| 8150 | CATCCACTCTTCCAACCTTTCAG | 9414 | CTGAAAGGTTGGAAGAGTGGATG |
| 8151 | CCATCCACTCTTCCAACCTTTCA | 9415 | TGAAAGGTTGGAAGAGTGGATGG |
| 8152 | CCCATCCACTCTTCCAACCTTTC | 9416 | GAAAGGTTGGAAGAGTGGATGGG |
| 8153 | ACCCATCCACTCTTCCAACCTTT | 9417 | AAAGGTTGGAAGAGTGGATGGGT |
| 8154 | CACCCATCCACTCTTCCAACCTT | 9418 | AAGGTTGGAAGAGTGGATGGGTG |
| 8155 | TCACCCATCCACTCTTCCAACCT | 9419 | AGGTTGGAAGAGTGGATGGGTGA |
| 8156 | TTCACCCATCCACTCTTCCAACC | 9420 | GGTTGGAAGAGTGGATGGGTGAA |
| 8157 | TTTCACCCATCCACTCTTCCAAC | 9421 | GTTGGAAGAGTGGATGGGTGAAA |
| 8158 | ATTTCACCCATCCACTCTTCCAA | 9422 | TTGGAAGAGTGGATGGGTGAAAT |
| 8159 | AATTTCACCCATCCACTCTTCCA | 9423 | TGGAAGAGTGGATGGGTGAAATT |
| 8160 | AAATTTCACCCATCCACTCTTCC | 9424 | GGAAGAGTGGATGGGTGAAATTT |
| 8161 | CAAATTTCACCCATCCACTCTTC | 9425 | GAAGAGTGGATGGGTGAAATTTG |
| 8162 | CCAAATTTCACCCATCCACTCTT | 9426 | AAGAGTGGATGGGTGAAATTTGG |
| 8163 | CCCAAATTTCACCCATCCACTCT | 9427 | AGAGTGGATGGGTGAAATTTGGG |
| 8164 | CCCCAAATTTCACCCATCCACTC | 9428 | GAGTGGATGGGTGAAATTTGGGG |
| 8165 | ACCCCAAATTTCACCCATCCACT | 9429 | AGTGGATGGGTGAAATTTGGGGT |
| 8166 | TACCCCAAATTTCACCCATCCAC | 9430 | GTGGATGGGTGAAATTTGGGGTA |
| 8167 | CTACCCCAAATTTCACCCATCCA | 9431 | TGGATGGGTGAAATTTGGGGTAG |
| 8168 | ACTACCCCAAATTTCACCCATCC | 9432 | GGATGGGTGAAATTTGGGGTAGT |
| 8169 | AACTACCCCAAATTTCACCCATC | 9433 | GATGGGTGAAATTTGGGGTAGTT |
| 8170 | TAACTACCCCAAATTTCACCCAT | 9434 | ATGGGTGAAATTTGGGGTAGTTA |
| 8171 | CTAACTACCCCAAATTTCACCCA | 9435 | TGGGTGAAATTTGGGGTAGTTAG |
| 8172 | TCTAACTACCCCAAATTTCACCC | 9436 | GGGTGAAATTTGGGGTAGTTAGA |
| 8173 | ATCTAACTACCCCAAATTTCACC | 9437 | GGTGAAATTTGGGGTAGTTAGAT |
| 8174 | CATCTAACTACCCCAAATTTCAC | 9438 | GTGAAATTTGGGGTAGTTAGATG |
| 8175 | CCATCTAACTACCCCAAATTTCA | 9439 | TGAAATTTGGGGTAGTTAGATGG |
| 8176 | CCCATCTAACTACCCCAAATTTC | 9440 | GAAATTTGGGGTAGTTAGATGGG |
| 8177 | ACCCATCTAACTACCCCAAATTT | 9441 | AAATTTGGGGTAGTTAGATGGGT |
| 8178 | CACCCATCTAACTACCCCAAATT | 9442 | AATTTGGGGTAGTTAGATGGGTG |
| 8179 | CCACCCATCTAACTACCCCAAAT | 9443 | ATTTGGGGTAGTTAGATGGGTGG |
| 8180 | CCCACCCATCTAACTACCCCAAA | 9444 | TTTGGGGTAGTTAGATGGGTGGG |
| 8181 | ACCCACCCATCTAACTACCCCAA | 9445 | TTGGGGTAGTTAGATGGGTGGGT |
| 8182 | CACCCACCCATCTAACTACCCCA | 9446 | TGGGGTAGTTAGATGGGTGGGTG |
| 8183 | ACACCCACCCATCTAACTACCCC | 9447 | GGGGTAGTTAGATGGGTGGGTGT |
| 8184 | CACACCCACCCATCTAACTACCC | 9448 | GGGTAGTTAGATGGGTGGGTGTG |
| 8185 | ACACACCCACCCATCTAACTACC | 9449 | GGTAGTTAGATGGGTGGGTGTGT |
| 8186 | CACACACCCACCCATCTAACTAC | 9450 | GTAGTTAGATGGGTGGGTGTGTG |
| 8187 | CCACACACCCACCCATCTAACTA | 9451 | TAGTTAGATGGGTGGGTGTGTGG |
| 8188 | TCCACACACCCACCCATCTAACT | 9452 | AGTTAGATGGGTGGGTGTGTGGA |
| 8189 | ATCCACACACCCACCCATCTAAC | 9453 | GTTAGATGGGTGGGTGTGTGGAT |
| 8190 | CATCCACACACCCACCCATCTAA | 9454 | TTAGATGGGTGGGTGTGTGGATG |
| 8191 | CCATCCACACACCCACCCATCTA | 9455 | TAGATGGGTGGGTGTGTGGATGG |
| 8192 | TCCATCCACACACCCACCCATCT | 9456 | AGATGGGTGGGTGTGTGGATGGA |
| 8193 | ATCCATCCACACACCCACCCATC | 9457 | GATGGGTGGGTGTGTGGATGGAT |
| 8194 | TATCCATCCACACACCCACCCAT | 9458 | ATGGGTGGGTGTGTGGATGGATA |
| 8195 | TTATCCATCCACACACCCACCCA | 9459 | TGGGTGGGTGTGTGGATGGATAA |
| 8196 | TTTATCCATCCACACACCCACCC | 9460 | GGGTGGGTGTGTGGATGGATAAA |
| 8197 | TTTTATCCATCCACACACCCACC | 9461 | GGTGGGTGTGTGGATGGATAAAA |
| 8198 | CTTTTATCCATCCACACACCCAC | 9462 | GTGGGTGTGTGGATGGATAAAAG |
| 8199 | TCTTTTATCCATCCACACACCCA | 9463 | TGGGTGTGTGGATGGATAAAAGA |
| 8200 | CTCTTTTATCCATCCACACACCC | 9464 | GGGTGTGTGGATGGATAAAAGAG |
| 8201 | ACTCTTTTATCCATCCACACACC | 9465 | GGTGTGTGGATGGATAAAAGAGT |
| 8202 | TACTCTTTTATCCATCCACACAC | 9466 | GTGTGTGGATGGATAAAAGAGTA |
| 8203 | CTACTCTTTTATCCATCCACACA | 9467 | TGTGTGGATGGATAAAAGAGTAG |
| 8204 | TCTACTCTTTTATCCATCCACAC | 9468 | GTGTGGATGGATAAAAGAGTAGA |
| 8205 | ATCTACTCTTTTATCCATCCACA | 9469 | TGTGGATGGATAAAAGAGTAGAT |
| 8206 | CATCTACTCTTTTATCCATCCAC | 9470 | GTGGATGGATAAAAGAGTAGATG |
| 8207 | TCATCTACTCTTTTATCCATCCA | 9471 | TGGATGGATAAAAGAGTAGATGA |
| 8208 | TTCATCTACTCTTTTATCCATCC | 9472 | GGATGGATAAAAGAGTAGATGAA |
| 8209 | ATTCATCTACTCTTTTATCCATC | 9473 | GATGGATAAAAGAGTAGATGAAT |
| 8210 | CATTCATCTACTCTTTTATCCAT | 9474 | ATGGATAAAAGAGTAGATGAATG |
| 8211 | TCATTCATCTACTCTTTTATCCA | 9475 | TGGATAAAAGAGTAGATGAATGA |
| 8212 | TTCATTCATCTACTCTTTTATCC | 9476 | GGATAAAAGAGTAGATGAATGAA |
| 8213 | ATTCATTCATCTACTCTTTTATC | 9477 | GATAAAAGAGTAGATGAATGAAT |
| 8214 | AATTCATTCATCTACTCTTTTAT | 9478 | ATAAAAGAGTAGATGAATGAATT |
| 8215 | TAATTCATTCATCTACTCTTTTA | 9479 | TAAAAGAGTAGATGAATGAATTA |
| 8216 | TTAATTCATTCATCTACTCTTTT | 9480 | AAAAGAGTAGATGAATGAATTAA |
| 8217 | ATTAATTCATTCATCTACTCTTT | 9481 | AAAGAGTAGATGAATGAATTAAT |
| 8218 | CATTAATTCATTCATCTACTCTT | 9482 | AAGAGTAGATGAATGAATTAATG |
| 8219 | TCATTAATTCATTCATCTACTCT | 9483 | AGAGTAGATGAATGAATTAATGA |
| 8220 | TTCATTAATTCATTCATCTACTC | 9484 | GAGTAGATGAATGAATTAATGAA |
| 8221 | ATTCATTAATTCATTCATCTACT | 9485 | AGTAGATGAATGAATTAATGAAT |
| 8222 | TATTCATTAATTCATTCATCTAC | 9486 | GTAGATGAATGAATTAATGAATA |
| 8223 | TTATTCATTAATTCATTCATCTA | 9487 | TAGATGAATGAATTAATGAATAA |
| 8224 | TTTATTCATTAATTCATTCATCT | 9488 | AGATGAATGAATTAATGAATAAA |
| 8225 | GTTTATTCATTAATTCATTCATC | 9489 | GATGAATGAATTAATGAATAAAC |
| 8226 | TGTTTATTCATTAATTCATTCAT | 9490 | ATGAATGAATTAATGAATAAACA |
| 8227 | CTGTTTATTCATTAATTCATTCA | 9491 | TGAATGAATTAATGAATAAACAG |
| 8228 | CCTGTTTATTCATTAATTCATTC | 9492 | GAATGAATTAATGAATAAACAGG |
| 8229 | GCCTGTTTATTCATTAATTCATT | 9493 | AATGAATTAATGAATAAACAGGC |
| 8230 | TGCCTGTTTATTCATTAATTCAT | 9494 | ATGAATTAATGAATAAACAGGCA |
| 8231 | CTGCCTGTTTATTCATTAATTCA | 9495 | TGAATTAATGAATAAACAGGCAG |
| 8232 | TCTGCCTGTTTATTCATTAATTC | 9496 | GAATTAATGAATAAACAGGCAGA |
| 8233 | ATCTGCCTGTTTATTCATTAATT | 9497 | AATTAATGAATAAACAGGCAGAT |
| 8234 | CATCTGCCTGTTTATTCATTAAT | 9498 | ATTAATGAATAAACAGGCAGATG |
| 8235 | CCATCTGCCTGTTTATTCATTAA | 9499 | TTAATGAATAAACAGGCAGATGG |
| 8236 | TCCATCTGCCTGTTTATTCATTA | 9500 | TAATGAATAAACAGGCAGATGGA |
| 8237 | ATCCATCTGCCTGTTTATTCATT | 9501 | AATGAATAAACAGGCAGATGGAT |
| 8238 | CATCCATCTGCCTGTTTATTCAT | 9502 | ATGAATAAACAGGCAGATGGATG |
| 8239 | TCATCCATCTGCCTGTTTATTCA | 9503 | TGAATAAACAGGCAGATGGATGA |
| 3710 | ATCATCCATCTGCCTGTTTATTC | 9504 | GAATAAACAGGCAGATGGATGAT |
| 3711 | CATCATCCATCTGCCTGTTTATT | 9505 | AATAAACAGGCAGATGGATGATG |
| 3712 | ACATCATCCATCTGCCTGTTTAT | 9506 | ATAAACAGGCAGATGGATGATGT |
| 3713 | TACATCATCCATCTGCCTGTTTA | 9507 | TAAACAGGCAGATGGATGATGTA |
| 3714 | TTACATCATCCATCTGCCTGTTT | 9508 | AAACAGGCAGATGGATGATGTAA |
| 3715 | CTTACATCATCCATCTGCCTGTT | 9509 | AACAGGCAGATGGATGATGTAAG |
| 3716 | GCTTACATCATCCATCTGCCTGT | 9510 | ACAGGCAGATGGATGATGTAAGC |
| 3717 | AGCTTACATCATCCATCTGCCTG | 9511 | CAGGCAGATGGATGATGTAAGCT |
| 3718 | CAGCTTACATCATCCATCTGCCT | 9512 | AGGCAGATGGATGATGTAAGCTG |
| 3719 | GCAGCTTACATCATCCATCTGCC | 9513 | GGCAGATGGATGATGTAAGCTGC |
| 3720 | GGCAGCTTACATCATCCATCTGC | 9514 | GCAGATGGATGATGTAAGCTGCC |
| 3721 | GGGCAGCTTACATCATCCATCTG | 9515 | CAGATGGATGATGTAAGCTGCCC |
| 3722 | GGGGCAGCTTACATCATCCATCT | 9516 | AGATGGATGATGTAAGCTGCCCC |
| 3723 | TGGGGCAGCTTACATCATCCATC | 9517 | GATGGATGATGTAAGCTGCCCCA |
| 3724 | CTGGGGCAGCTTACATCATCCAT | 9518 | ATGGATGATGTAAGCTGCCCCAG |
| 3725 | TCTGGGGCAGCTTACATCATCCA | 9519 | TGGATGATGTAAGCTGCCCCAGA |
| 3726 | GTCTGGGGCAGCTTACATCATCC | 9520 | GGATGATGTAAGCTGCCCCAGAC |
| 3727 | GGTCTGGGGCAGCTTACATCATC | 9521 | GATGATGTAAGCTGCCCCAGACC |
| 3728 | GGGTCTGGGGCAGCTTACATCAT | 9522 | ATGATGTAAGCTGCCCCAGACCC |
| 3729 | AGGGTCTGGGGCAGCTTACATCA | 9523 | TGATGTAAGCTGCCCCAGACCCT |
| 3730 | CAGGGTCTGGGGCAGCTTACATC | 9524 | GATGTAAGCTGCCCCAGACCCTG |
| 3731 | CCAGGGTCTGGGGCAGCTTACAT | 9525 | ATGTAAGCTGCCCCAGACCCTGG |
| 3732 | CCCAGGGTCTGGGGCAGCTTACA | 9526 | TGTAAGCTGCCCCAGACCCTGGG |
| 3733 | TCCCAGGGTCTGGGGCAGCTTAC | 9527 | GTAAGCTGCCCCAGACCCTGGGA |
| 3734 | GTCCCAGGGTCTGGGGCAGCTTA | 9528 | TAAGCTGCCCCAGACCCTGGGAC |
| 3735 | GGTCCCAGGGTCTGGGGCAGCTT | 9529 | AAGCTGCCCCAGACCCTGGGACC |
| 3736 | AGGTCCCAGGGTCTGGGGCAGCT | 9530 | AGCTGCCCCAGACCCTGGGACCT |
| 3737 | GAGGTCCCAGGGTCTGGGGCAGC | 9531 | GCTGCCCCAGACCCTGGGACCTC |
| 3738 | AGAGGTCCCAGGGTCTGGGGCAG | 9532 | CTGCCCCAGACCCTGGGACCTCT |
| 3739 | CAGAGGTCCCAGGGTCTGGGGCA | 9533 | TGCCCCAGACCCTGGGACCTCTG |
| 3740 | TCAGAGGTCCCAGGGTCTGGGGC | 9534 | GCCCCAGACCCTGGGACCTCTGA |
| 3741 | GTCAGAGGTCCCAGGGTCTGGGG | 9535 | CCCCAGACCCTGGGACCTCTGAC |
| 3742 | GGTCAGAGGTCCCAGGGTCTGGG | 9536 | CCCAGACCCTGGGACCTCTGACC |
| 3743 | GGGTCAGAGGTCCCAGGGTCTGG | 9537 | CCAGACCCTGGGACCTCTGACCC |
| 3744 | GGGGTCAGAGGTCCCAGGGTCTG | 9538 | CAGACCCTGGGACCTCTGACCCC |
| 3745 | GGGGGTCAGAGGTCCCAGGGTCT | 9539 | AGACCCTGGGACCTCTGACCCCC |
| 3746 | CGGGGGTCAGAGGTCCCAGGGTC | 9540 | GACCCTGGGACCTCTGACCCCCG |
| 3747 | CCGGGGGTCAGAGGTCCCAGGGT | 9541 | ACCCTGGGACCTCTGACCCCCGG |
| 3748 | GCCGGGGGTCAGAGGTCCCAGGG | 9542 | CCCTGGGACCTCTGACCCCCGGC |
| 3749 | CGCCGGGGGTCAGAGGTCCCAGG | 9543 | CCTGGGACCTCTGACCCCCGGCG |
| 3750 | TCGCCGGGGGTCAGAGGTCCCAG | 9544 | CTGGGACCTCTGACCCCCGGCGA |
| 3751 | GTCGCCGGGGGTCAGAGGTCCCA | 9545 | TGGGACCTCTGACCCCCGGCGAC |
| 3752 | GGTCGCCGGGGGTCAGAGGTCCC | 9546 | GGGACCTCTGACCCCCGGCGACC |
| 3753 | GGGTCGCCGGGGGTCAGAGGTCC | 9547 | GGACCTCTGACCCCCGGCGACCC |
| 3754 | GGGGTCGCCGGGGGTCAGAGGTC | 9548 | GACCTCTGACCCCCGGCGACCCC |
| 3755 | AGGGGTCGCCGGGGGTCAGAGGT | 9549 | ACCTCTGACCCCCGGCGACCCCT |
| 3756 | AAGGGGTCGCCGGGGGTCAGAGG | 9550 | CCTCTGACCCCCGGCGACCCCTT |
| 3757 | CAAGGGGTCGCCGGGGGTCAGAG | 9551 | CTCTGACCCCCGGCGACCCCTTG |
| 3758 | GCAAGGGGTCGCCGGGGGTCAGA | 9552 | TCTGACCCCCGGCGACCCCTTGC |
| 3759 | TGCAAGGGGTCGCCGGGGGTCAG | 9553 | CTGACCCCCGGCGACCCCTTGCA |
| 3760 | GTGCAAGGGGTCGCCGGGGGTCA | 9554 | TGACCCCCGGCGACCCCTTGCAC |
| 3761 | AGTGCAAGGGGTCGCCGGGGGTC | 9555 | GACCCCCGGCGACCCCTTGCACT |
| 3762 | GAGTGCAAGGGGTCGCCGGGGGT | 9556 | ACCCCCGGCGACCCCTTGCACTC |
| 3763 | AGAGTGCAAGGGGTCGCCGGGGG | 9557 | CCCCCGGCGACCCCTTGCACTCT |
| 3764 | GAGAGTGCAAGGGGTCGCCGGGG | 9558 | CCCCGGCGACCCCTTGCACTCTC |
| 3765 | GGAGAGTGCAAGGGGTCGCCGGG | 9559 | CCCGGCGACCCCTTGCACTCTCC |
| 3766 | TGGAGAGTGCAAGGGGTCGCCGG | 9560 | CCGGCGACCCCTTGCACTCTCCA |
| 3767 | ATGGAGAGTGCAAGGGGTCGCCG | 9561 | CGGCGACCCCTTGCACTCTCCAT |
| 3768 | CATGGAGAGTGCAAGGGGTCGCC | 9562 | GGCGACCCCTTGCACTCTCCATG |
| 3769 | TCATGGAGAGTGCAAGGGGTCGC | 9563 | GCGACCCCTTGCACTCTCCATGA |
| 3770 | GTCATGGAGAGTGCAAGGGGTCG | 9564 | CGACCCCTTGCACTCTCCATGAC |
| 3771 | TGTCATGGAGAGTGCAAGGGGTC | 9565 | GACCCCTTGCACTCTCCATGACA |
| 3772 | GTGTCATGGAGAGTGCAAGGGGT | 9566 | ACCCCTTGCACTCTCCATGACAC |
| 3773 | AGTGTCATGGAGAGTGCAAGGGG | 9567 | CCCCTTGCACTCTCCATGACACT |
| 3774 | AAGTGTCATGGAGAGTGCAAGGG | 9568 | CCCTTGCACTCTCCATGACACTT |
| 3775 | AAAGTGTCATGGAGAGTGCAAGG | 9569 | CCTTGCACTCTCCATGACACTTT |
| 3776 | GAAAGTGTCATGGAGAGTGCAAG | 9570 | CTTGCACTCTCCATGACACTTTC |
| 3777 | AGAAAGTGTCATGGAGAGTGCAA | 9571 | TTGCACTCTCCATGACACTTTCT |
| 3778 | GAGAAAGTGTCATGGAGAGTGCA | 9572 | TGCACTCTCCATGACACTTTCTC |
| 3779 | AGAGAAAGTGTCATGGAGAGTGC | 9573 | GCACTCTCCATGACACTTTCTCT |
| 3780 | GAGAGAAAGTGTCATGGAGAGTG | 9574 | CACTCTCCATGACACTTTCTCTC |
| 3781 | GGAGAGAAAGTGTCATGGAGAGT | 9575 | ACTCTCCATGACACTTTCTCTCC |
| 3782 | GGGAGAGAAAGTGTCATGGAGAG | 9576 | CTCTCCATGACACTTTCTCTCCC |
| 3783 | TGGGAGAGAAAGTGTCATGGAGA | 9577 | TCTCCATGACACTTTCTCTCCCA |
| 3784 | ATGGGAGAGAAAGTGTCATGGAG | 9578 | CTCCATGACACTTTCTCTCCCAT |
| 3785 | CATGGGAGAGAAAGTGTCATGGA | 9579 | TCCATGACACTTTCTCTCCCATG |
| 3786 | CCATGGGAGAGAAAGTGTCATGG | 9580 | CCATGACACTTTCTCTCCCATGG |
| 3787 | ACCATGGGAGAGAAAGTGTCATG | 9581 | CATGACACTTTCTCTCCCATGGT |
| 3788 | CACCATGGGAGAGAAAGTGTCAT | 9582 | ATGACACTTTCTCTCCCATGGTG |
| 3789 | CCACCATGGGAGAGAAAGTGTCA | 9583 | TGACACTTTCTCTCCCATGGTGG |
| 3790 | GCCACCATGGGAGAGAAAGTGTC | 9584 | GACACTTTCTCTCCCATGGTGGC |
| 3791 | TGCCACCATGGGAGAGAAAGTGT | 9585 | ACACTTTCTCTCCCATGGTGGCA |
| 3792 | CTGCCACCATGGGAGAGAAAGTG | 9586 | CACTTTCTCTCCCATGGTGGCAG |

*At least one nucleoside linkage of the nucleobase sequence is selected from a
phosphorothioate linkage, an alkyl phosphate linkage, a phosphorodithioate linkage,
a phosphotriester linkage, an alkylphosphonate linkage, a 3-methoxypropyl
phosphonate linkage, a methylphosphonate linkage, an aminoalkylphosphotriester
linkage, an alkylene phosphonate linkage, a phosphinate linkage, a phosphoramidate
linkage, a phosphoramidothioate linkage, a thiophosphorodiamidate linkage, a
phosphorodiamidate (e.g., comprising a phosphorodiamidate morpholino (PMO), 3′
amino ribose, or 5′ amino ribose) linkage, an aminoalkylphosphoramidate linkage, a
thiophosphoramidate linkage, a thionoalkylphosphonate linkage, a thionoalkyl-
phosphotriester linkage, a thiophosphate linkage, a selenophosphate linkage, and
a boranophosphate linkage.

### SMN2 Oligonucleotides Complementary to SMN2 Transcript

In some embodiments, a SMN2 AON includes a sequence that is at least 85% complementary to a sequence that shares at least 90% (e.g., 90%, 91%, 92%, 93%, 94%, 95%, 96%, 97%, 98%, 99%, or 100%) identity to a region of a SMN2 transcript (e.g., any one of SEQ ID NOs: 9698-9709). In some embodiments, a SMN2 AON includes a sequence that is between 90-95% complementary to a sequence that shares at least 90% (e.g., 90%, 91%, 92%, 93%, 94%, 95%, 96%, 97%, 98%, 99%, or 100%) identity to a region of a SMN2 transcript (e. g., any one of SEQ ID NOs: 9698-9709). In particular embodiments, a SMN2 AON includes a sequence that is between 85% and 90% complementary to a sequence that shares at least 90% (e.g., 90%, 91%, 92%, 93%, 94%, 95%, 96%, 97%, 98%, 99%, or 100%) identity to a region of a SMN2 transcript (e.g., any one of SEQ ID NOs: 9698-9709). In particular embodiments, a SMN2 AON includes a sequence that is between 84% to 88% complementary to a sequence that shares at least 90% (e.g., 90%, 91%, 92%, 93%, 94%, 95%, 96%, 97%, 98%, 99%, or 100%) identity to a region of a SMN2 transcript (e.g., any one of SEQ ID NOs: 9698-9709). In particular embodiments, a SMN2 AON includes a sequence that is between 89% to 92% complementary to a sequence that shares at least 90% (e.g., 90%, 91%, 92%, 93%, 94%, 95%, 96%, 97%, 98%, 99%, or 100%) identity to a region of a SMN2 transcript (e.g., any one of SEQ ID NOs: 9698-9709). In particular embodiments, a SMN2 AON includes a sequence that is between 94% to 96% complementary to a sequence that shares at least 90% (e.g., 90%, 91%, 92%, 93%, 94%, 95%, 96%, 97%, 98%, 99%, or 100%) identity to a region of a SMN2 transcript e.g., SEQ ID NOs: 9698-9709).

In various embodiments, a SMN2 AON comprises a sequence that shares at least 85% identity with an equal length portion of any one of SEQ ID NOs: 9710-10141 and SEQ ID NOs: 10574-10651. In various embodiments, a SMN2 AON comprises a sequence that shares at least 90% identity with an equal length portion of any one of SEQ ID NOs: 9710-10141 and SEQ ID NOs: 10574-10651.

In some embodiments, the SMN2 AON comprises a spacer and has a segment having at most 7 linked nucleosides. In some embodiments, the SMN2 AON comprises a spacer and has a segment having at most 6, 5, 4, 3, or 2 linked nucleosides.

SMN2 AON binding specificity can be assessed via measurement of parameters such as dissociation constant, melting temperature or other criteria such as changes in protein or RNA expression levels or other assays that measure SMN2 activity or expression.

In some embodiments, a SMN2 AON can include a non-duplexed oligonucleotide. In some embodiments, a SMN2 AON can include a duplex of two oligonucleotides where the first oligonucleotide includes a nucleobase sequence that is completely or almost completely complementary to a SMN2 pre-mRNA sequence and the second oligonucleotide includes a nucleobase sequence that is complementary to the nucleobase sequence of the first oligonucleotide.

In some embodiments, a SMN2 AON can target SMN2 mRNAs of one or more isoforms. In some embodiments, the SMN2 AON includes a nucleobase sequence that is complementary to a nucleobase sequence of a SMN2 gene or a SMN2 mRNA.

### SMN2 Transcript

In various embodiments, a SMN2 transcript comprises the sequence provided as SEQ ID NO: 9698.

(SEQ ID NO: 9698
GCACCCGCGGGTTTGCTATGGCGATGAGCAGCGGCGGCAGTGGTGGCGGC
GTCCCGGAGCAGGAGGATTCCGTGCTGTTCCGGCGCGGCACAGGCCAGAG
CGATGATTCTGACATTTGGGATGATACAGCACTGATAAAAGCATATGATA
AAGCTGTGGCTTCATTTAAGCATGCTCTAAAGAATGGTGACATTTGTGAA
ACTTCGGGTAAACCAAAAACCACACCTAAAAGAAAACCTGCTAAGAAGAA
TAAAAGCCAAAAGAAGAATACTGCAGCTTCCTTACAACAGTGGAAAGTTG
GGGACAAATGTTCTGCCATTTGGTCAGAAGACGGTTGCATTTACCCAGCT
ACCATTGCTTCAATTGATTTTAAGAGAGAAACCTGTGTTGTGGTTTACAC
TGGATATGGAAATAGAGAGGAGCAAAATCTGTCCGATCTACTTTCCCCAA
TCTGTGAAGTAGCTAATAATATAGAACAAAATGCTCAAGAGAATGAAAAT
GAAAGCCAAGTTTCAACAGATGAAAGTGAGAACTCCAGGTCTCCTGGAAA
TAAATCAGATAACATCAAGCCCAAATCTGCTCCATGGAACTCTTTTCTCC
CTCCACCACCCCCCATGCCAGGGCCAAGACTGGGACCAGGAAAGCCAGGT
CTAAAATTCAATGGCCCACCACCGCCACCGCCACCACCACCACCCCACTT
ACTATCATGCTGGCTGCCTCCATTTCCTTCTGGACCACCAATAATTCCCC
CACCACCTCCCATATGTCCAGATTCTCTTGATGATGCTGATGCTTTGGGA
AGTATGTTAATTTCATGGTACATGAGTGGCTATCATACTGGCTATTATAT
GGGTTTTAGACAAAATCAAAAAGAAGGAAGGTGCTCACATTCCTTAAATT
AAGGAGAAATGCTGGCATAGAGCAGCACTAAATGACACCACTAAAGAAAC
GATCAGACAGATCTGGAATGTGAAGCGTTATAGAAGATAACTGGCCTCAT
TTCTTCAAAATATCAAGTGTTGGGAAAGAAAAAAGGAAGTGGAATGGGTA
ACTCTTCTTGATTAAAAGTTATGTAATAACCAAATGCAATGTGAAATATT
TTACTGGACTCTATTTTGAAAAACCATCTGTAAAAGACTGAGGTGGGGGT
GGGAGGCCAGCACGGTGGTGAGGCAGTTGAGAAAATTTGAATGTGGATTA
GATTTTGAATGATATTGGATAATTATTGGTAATTTTATGAGCTGTGAAAG
GGTGTTGTAGTTTATAAAAGACTGTCTTAATTTGCATACTTAAGCATTTA
GGAATGAAGTGTTAGAGTGTCTTAAAATGTTTCAAATGGTTTAACAAAAT
GTATGTGAGGCGTATGTGGCAAAATGTTACAGAATCTAACTGGTGGACAT
GGCTGTTCATTGTACTGTTTTTTTCTATCTTCTATATGTTTAAAAGTATA
TAATAAAAATATTTAATTTTTTTTTAAATTA
(SOURCE NCBI REFERENCE NO: NM_017411.4)

In various embodiments, a SMN2 mRNA transcript comprises the sequence provided as SEQ ID NO: 9699.

(SEQ ID NO: 9699)
GCACCCGCGGGTTTGCTATGGCGATGAGCAGCGGCGGCAGTGGTGGCGGC
GTCCCGGAGCAGGAGGATTCCGTGCTGTTCCGGCGCGGCACAGGCCAGAG
CGATGATTCTGACATTTGGGATGATACAGCACTGATAAAAGCATATGATA
AAGCTGTGGCTTCATTTAAGCATGCTCTAAAGAATGGTGACATTTGTGAA
ACTTCGGGTAAACCAAAAACCACACCTAAAAGAAAACCTGCTAAGAAGAA
TAAAAGCCAAAAGAAGAATACTGCAGCTTCCTTACAACAGTGGAAAGTTG
GGGACAAATGTTCTGCCATTTGGTCAGAAGACGGTTGCATTTACCCAGCT
ACCATTGCTTCAATTGATTTTAAGAGAGAAACCTGTGTTGTGGTTTACAC
TGGATATGGAAATAGAGAGGAGCAAAATCTGTCCGATCTACTTTCCCCAA
TCTGTGAAGTAGCTAATAATATAGAACAAAATGCTCAAGAGAATGAAAAT
GAAAGCCAAGTTTCAACAGATGAAAGTGAGAACTCCAGGTCTCCTGGAAA
TAAATCAGATAACATCAAGCCCAAATCTGCTCCATGGAACTCTTTTCTCC
CTCCACCACCCCCCATGCCAGGGCCAAGACTGGGACCAGGAAAGCCAGGT
CTAAAATTCAATGGCCCACCACCGCCACCGCCACCACCACCACCCCACTT
ACTATCATGCTGGCTGCCTCCATTTCCTTCTGGACCACCAATAATTCCCC
CACCACCTCCCATATGTCCAGATTCTCTTGATGATGCTGATGCTTTGGGA
AGTATGTTAATTTCATGGTACATGAGTGGCTATCATACTGGCTATTATAT
GGAAATGCTGGCATAGAGCAGCACTAAATGACACCACTAAAGAAACGATC
AGACAGATCTGGAATGTGAAGCGTTATAGAAGATAACTGGCCTCATTTCT
TCAAAATATCAAGTGTTGGGAAAGAAAAAAGGAAGTGGAATGGGTAACTC
TTCTTGATTAAAAGTTATGTAATAACCAAATGCAATGTGAAATATTTTAC
TGGACTCTATTTTGAAAAACCATCTGTAAAAGACTGAGGTGGGGGTGGGA
GGCCAGCACGGTGGTGAGGCAGTTGAGAAAATTTGAATGTGGATTAGATT
TTGAATGATATTGGATAATTATTGGTAATTTTATGAGCTGTGAGAAGGGT
GTTGTAGTTTATAAAAGACTGTCTTAATTTGCATACTTAAGCATTTAGGA
ATGAAGTGTTAGAGTGTCTTAAAATGTTTCAAATGGTTTAACAAAATGTA
TGTGAGGCGTATGTGGCAAAATGTTACAGAATCTAACTGGTGGACATGGC
TGTTCATTGTACTGTTTTTTTCTATCTTCTATATGTTTAAAAGTATATAA
TAAAAATATTTAATTTTTTTTTAAATTA
(Source: NCBI Reference Sequence: NM_022875.3).

In various embodiments, a SMN2 mRNA transcript comprises the sequence provided as SEQ ID NO: 9700.

(SEQ ID NO: 9700)
CCACAAATGTGGGAGGGCGATAACCACTCGTAGAAAGCGTGAGAAGTTAC
TACAAGCGGTCCTCCCGGCCACCGTACTGTTCCGCTCCCAGAAGCCCCGG
GCGGCGGAAGTCGTCACTCTTAAGAAGGGACGGGGCCCCACGCTGCGCAC
CCGCGGGTTTGCTATGGCGATGAGCAGCGGCGGCAGTGGTGGCGGCGTCC
CGGAGCAGGAGGATTCCGTGCTGTTCGGCGCGGCACAGGCCAGAGCGATG
ATTCTGACATTTGGGATGATACAGCACTGATAAAAGCATATGATAAAGCT
GTGGCTTCATTTAAGCATGCTCTAAAGAATGGTGACATTTGTGAAACTTC
GGGTAAACCAAAAACCACACCTAAAAGAAAACCTGCTAAGAAGAATAAAA
GCCAAAAGAAGAATACTGCAGCTTCCTTACAACAGTGGAAAGTTGGGGAC
AAATGTTCTGCCATTTGGTCAGAAGACGGTTGCATTTACCCAGCTACCAT
TGCTTCAATTGATTTTAAGAGAGAAACCTGTGTTGTGGTTTACACTGGAT
ATGGAAATAGAGAGGAGCAAAATCTGTCCGATCTACTTTCCCCAATCTGT
GAAGTAGCTAATAATATAGAACAAAATGCTCAAGAGAATGAAAATGAAAG
CCAAGTTTCAACAGATGAAAGTGAGAACTCCAGGTCTCCTGGAAATAAAT
CAGATAACATCAAGCCCAAATCTGCTCCATGGAACTCTTTTCTCCCTCCA
CCACCCCCCATGCCAGGGCCAAGACTGGGACCAGGAAAGATAATTCCCCC
ACCACCTCCCATATGTCCAGATTCTCTTGATGATGCTGATGCTTTGGGAA
GTATGTTAATTTCATGGTACATGAGTGGCTATCATACTGGCTATTATATG
GGTTTTAGACAAAATCAAAAAGAAGGAAGGTGCTCACATTCCTTAAATTA
AGGAGAAATGCTGGCATAGAGCAGCACTAAATGACACCACTAAAGAAACG
ATCAGACAGATCTGGAATGTGAAGCGTTATAGAAGATAACTGGCCTCATT
TCTTCAAAATATCAAGTGTTGGGAAAGAAAAAAGGAAGTGGAATGGGTAA
CTCTTCTTGATTAAAAGTTATGTAATAACCAAATGCAATGTGAAATATTT
TACTGGACTCTATTTTGAAAAACCATCTGTAAAAGACTGAGGTGGGGGTG
GGAGGCCAGCACGGTGGTGAGGCAGTTGAGAAAATTTGAATGTGGATTAG
ATTTTGAATGATATTGGATAATTATTGGTAATTTTATGAGCTGTGAGAAG
GGTGTTGTAGTTTATAAAAGACTGTCTTAATTTGCATACTTAAGCATTTA
GGAATGAAGTGTTAGAGTGTCTTAAAATGTTTCAAATGGTTTAACAAAAT
GTATGTGAGGCGTATGTGGCAAAATGTTACAGAATCTAACTGGTGGACAT
GGCTGTTCATTGTACTGTTTTTTTCTATCTTCTATATGTTTAAAAGTATA
TAATAAAAATATTTAATTTTTTTTTAAATTAAAAAAA
(Source: NCBI Reference Sequence: NM_022876.2).

In various embodiments, a SMN2 mRNA transcript comprises the sequence provided as SEQ ID NO: 9701.

(SEQ ID NO: 9701)
CCACAAATGTGGGAGGGCGATAACCACTCGTAGAAAGCGTGAGAAGTTAC
TACAAGCGGTCCTCCCGGCCACCGTACTGTTCCGCTCCCAGAAGCCCCGG
GCGGCGGAAGTCGTCACTCTTAAGAAGGGACGGGGCCCCACGCTGCGCAC
CCGCGGGTTTGCTATGGCGATGAGCAGCGGCGGCAGTGGTGGCGGCGTCC
CGGAGCAGGAGGATTCCGTGCTGTTCCGGCGCGGCACAGGCCAGAGCGAT
GATTCTGACATTTGGGATGATACAGCACTGATAAAAGCATATGATAAAGC
TGTGGCTTCATTTAAGCATGCTCTAAAGAATGGTGACATTTGTGAAACTT
CGGGTAAACCAAAAACCACACCTAAAAGAAAACCTGCTAAGAAGAATAAA
AGCCAAAAGAAGAATACTGCAGCTTCCTTACAACAGTGGAAAGTTGGGGA
CAAATGTTCTGCCATTTGGTCAGAAGACGGTTGCATTTACCCAGCTACCA
TTGCTTCAATTGATTTTAAGAGAGAAACCTGTGTTGTGGTTTACACTGGA
TATGGAAATAGAGAGGAGCAAAATCTGTCCGATCTACTTTCCCCAATCTG
TGAAGTAGCTAATAATATAGAACAAAATGCTCAAGAGAATGAAAATGAAA
GCCAAGTTTCAACAGATGAAAGTGAGAACTCCAGGTCTCCTGGAAATAAA
TCAGATAACATCAAGCCCAAATCTGCTCCATGGAACTCTTTTCTCCCTCC
ACCACCCCCCATGCCAGGGCCAAGACTGGGACCAGGAAAGATAATTCCCC
CACCACCTCCCATATGTCCAGATTCTCTTGATGATGCTGATGCTTTGGGA
AGTATGTTAATTTCATGGTACATGAGTGGCTATCATACTGGCTATTATAT
GGAAATGCTGGCATAGAGCAGCACTAAATGACACCACTAAAGAAACGATC
AGACAGATCTGGAATGTGAAGCGTTATAGAAGATAACTGGCCTCATTTCT
TCAAAATATCAAGTGTTGGGAAAGAAAAAAGGAAGTGGAATGGGTAACTC
TTCTTGATTAAAAGTTATGTAATAACCAAATGCAATGTGAAATATTTTAC
TGGACTCTATTTTGAAAAACCATCTGTAAAAGACTGAGGTGGGGGTGGGA
GGCCAGCACGGTGGTGAGGCAGTTGAGAAAATTTGAATGTGGATTAGATT
TTGAATGATATTGGATAATTATTGGTAATTTTATGAGCTGTGAGAAGGGT
GTTGTAGTTTATAAAAGACTGTCTTAATTTGCATACTTAAGCATTTAGGA
ATGAAGTGTTAGAGTGTCTTAAAATGTTTCAAATGGTTTAACAAAATGTA
TGTGAGGCGTATGTGGCAAAATGTTACAGAATCTAACTGGTGGACATGGC
TGTTCATTGTACTGTTTTTTTCTATCTTCTATATGTTTAAAAGTATATAA
TAAAAATATTTAATTTTTTTTTAAATTAAAAAAA
(Source: NCBI Reference Sequence: NM_022877.2).

In various embodiments, a SMN2 mRNA transcript comprises the sequence provided as SEQ ID NO: 9702.

(SEQ ID NO: 9702)
GGCGGAAGTCGTCACTCTTAAGAAGGGACGGGGCCCCACGCTGCGCACCC
GCGGGTTTGCTATGGCGATGAGCAGCGGCGGCAGTGGTGGCGGCGTCCCG
GAGCAGGAGGATTCCGTGCTGTTCCGGCGCGGCACAGGCCAGAGCGATGA
TTCTGACATTTGGGATGATACAGCACTGATAAAAGCATATGATAAAGCTG
TGGCTTCATTTAAGCATGCTCTAAAGAATGGTGACATTTGTGAAACTTCG
GGTAAACCAAAAACCACACCTAAAAGAAAACCTGCTAAGAAGAATAAAAG
CCAAAAGAAGAATACTGCAGCTTCCTTACAACAGAATGAAAATGAAAGCC
AAGTTTCAACAGATGAAAGTGAGAACTCCAGGTCTCCTGGAAATAAATCA
GATAACATCAAGCCCAAATCTGCTCCATGGAACTCTTTTCTCCCTCCACC
ACCCCCCATGCCAGGGCCAAGACTGGGACCAGGAAAGATAATTCCCCCAC
CACCTCCCATATGTCCAGATTCTCTTGATGATGCTGATGCTTTGGGAAGT
ATGTTAATTTCATGGTACATGAGTGGCTATCATACTGGCTATTATATGGG
TTTTAGACAAAATCAAAAAGAAGGAAGGTGCTCACATTCCTTAAATTAAG
GAGAAATGCTGGCATAGAGCAGCACTAAATGACACCACTAAAGAAACGAT
CAGACAGATCTGGAATGTGAAGCGTTATAGAAGATAACTGGCCTCATTTC
TTCAAAATATCAAGTGTTGGGAAAGAAAAAAGGAAGTGGAATGGGTAACT
CTTCTTGATTAAAAGTTATGTAATAACCAAATGCAATGTGAAATATTTTA
CTGGACTCTATTTTGAAAAACCATCTGTAAAAGACTGAGGTGGGGGGGGA
GGCCAGCACGGTGGTGAGGCAGTTGAGAAAATTTGAATGTGGATTAGATT
TTGAATGATATTGGATAATTATTGGTAATTTTATGAGCTGTGAGAAGGGT
GTTGTAGTTTATAAAAGACTGTCTTAATTTGCATACTTAAGCATTTAGGA
ATGAAGTGTTAGAGTGTCTTAAAATGTTTCAAATGGTTTAACAAAATGTA
TGTGAGGCGTATGTGGCAAAATGTTACAGAATCTAACTGGTGGACATGGC
TGTTCATTGTACTGTTTTTTTCTATCTTCTATATGTTTAAAAGTATATAA
TAAAAATATTTAATTTTTTTTTAAATTA
(Source: NCBI Reference Sequence: XM_011543602.3).

In various embodiments, SMN2 miRNA transcript comprises the sequence provided as SEQ ID NO: 9703.

(SEQ ID NO: 9703)
GGCGGAAGTCGTCACTCTTAAGAAGGGACGGGGCCCCACGCTGCGCACCC
GCGGGTTTGCTATGGCGATGAGCAGCGGCGGCAGTGGTGGCGGCGTCCCG
GAGCAGGAGGATTCCGTGCTGTTCCGGCGCGGCACAGGCCAGAGCGATGA
TTCTGACATTTGGGATGATACAGCACTGATAAAAGCATATGATAAAGCTG
TGGCTTCATTTAAGCATGCTCTAAAGAATGGTGACATTTGTGAAACTTCG
GGTAAACCAAAAACCACACCTAAAAGAAAACCTGCTAAGAAGAATAAAAG
CCAAAAGAAGAATACTGCAGCTTCCTTACAACAGAATGAAAATGAAAGCC
AAGTTTCAACAGATGAAAGTGAGAACTCCAGGTCTCCTGGAAATAAATCA
GATAACATCAAGCCCAAATCTGCTCCATGGAACTCTTTTCTCCCTCCACC
ACCCCCCATGCCAGGGCCAAGACTGGGACCAGGAAAGATAATTCCCCCAC
CACCTCCCATATGTCCAGATTCTCTTGATGATGCTGATGCTTTGGGAAGT
ATGTTAATTTCATGGTACATGAGTGGCTATCATACTGGCTATTATATGGA
AATGCTGGCATAGAGCAGCACTAAATGACACCACTAAAGAAACGATCAGA
CAGATCTGGAATGTGAAGCGTTATAGAAGATAACTGGCCTCATTTCTTCA
AAATATCAAGTGTTGGGAAAGAAAAAAGGAAGTGGAATGGGTAACTCTTC
TTGATTAAAAGTTATGTAATAACCAAATGCAATGTGAAATATTTTACTGG
ACTCTATTTTGAAAAACCATCTGTAAAAGACTGAGGTGGGGGGGGAGGCC
AGCACGGTGGTGAGGCAGTTGAGAAAATTTGAATGTGGATTAGATTTTGA
ATGATATTGGATAATTATTGGTAATTTTATGAGCTGTGAGAAGGGTGTTG
TAGTTTATAAAAGACTGTCTTAATTTGCATACTTAAGCATTTAGGAATGA
AGTGTTAGAGTGTCTTAAAATGTTTCAAATGGTTTAACAAAATGTATGTG
AGGCGTATGTGGCAAAATGTTACAGAATCTAACTGGTGGACATGGCTGTT
CATTGTACTGTTTTTTTCTATCTTCTATATGTTTAAAAGTATATAATAAA
AATATTTAATTTTTTTTTAAATTA
(Source: NCBI Reference Sequence: XM_011543603.3).

In various embodiments, a SMN2 mRNA transcript comprises the sequence provided as SEQ ID NO: 9704.

(SEQ ID NO: 9704)
ACTCTTAAGAAGGGACGGGGCCCCACGCTGCGCACCCGCGGGTTTGCTAT
GGCGATGAGCAGCGGCGGCAGTGGTGGCGGCGTCCCGGAGCAGGAGGATT
CCGTGCTGTTCCGGCGCGGCACAGGCCAGAGCGATGATTCTGACATTTGG
GATGATACAGCACTGATAAAAGCATATGATAAAGCTGTGGCTTCATTTAA
GCATGCTCTAAAGAATGGTGACATTTGTGAAACTTCGGGTAAACCAAAAA
CCACACCTAAAAGAAAACCTGCTAAGAAGAATAAAAGCCAAAAGAAGAAT
ACTGCAGCTTCCTTACAACAGAATGAAAATGAAAGCCAAGTTTCAACAGA
TGAAAGTGAGAACTCCAGGTCTCCTGGAAATAAATCAGATAACATCAAGC
CCAAATCTGCTCCATGGAACTCTTTTCTCCCTCCACCACCCCCCATGCCA
GGGCCAAGACTGGGACCAGGAAAGCCAGGTCTAAAATTCAATGGCCCACC
ACCGCCACCGCCACCACCACCACCCCACTTACTATCATGCTGGCTGCCTC
CATTTCCTTCTGGACCACCAATAATTCCCCCACCACCTCCCATATGTCCA
GATTCTCTTGATGATGCTGATGCTTTGGGAAGTATGTTAATTTCATGGTA
CATGAGTGGCTATCATACTGGCTATTATATGGGTTTTAGACAAAATCAAA
AAGAAGGAAGGTGCTCACATTCCTTAAATTAAGGAGAAATGCTGGCATAG
AGCAGCACTAAATGACACCACTAAAGAAACGATCAGACAGATCTGGAATG
TGAAGCGTTATAGAAGATAACTGGCCTCATTTCTTCAAAATATCAAGTGT
TGGGAAAGAAAAAAGGAAGTGGAATGGGTAACTCTTCTTGATTAAAAGTT
ATGTAATAACCAAATGCAATGTGAAATATTTTACTGGACTCTATTTTGAA
AAACCATCTGTAAAAGACTGAGGTGGGGGTGGGAGGCCAGCACGGTGGTG
AGGCAGTTGAGAAAATTTGAATGTGGATTAGATTTTGAATGATATTGGAT
AATTATTGGTAATTTTATGAGCTGTGAGAAGGGTGTTGTAGTTTATAAAA
GACTGTCTTAATTTGCATACTTAAGCATTTAGGAATGAAGTGTTAGAGTG
TCTTAAAATGTTTCAAATGGTTTAACAAAATGTATGTGAGGCGTATGTGG
CAAAATGTTACAGAATCTAACTGGTGGACATGGCTGTTCATTGTACTGTT
TTTTTCTATCTTCTATATGTTTAAAAGTATATAATAAAAATATTTAATTT
TTTTTTAAATTA
(Source: NCBI Reference Sequence: XM_011543600.2

In various embodiments, a SMN2 mRNA transcript comprises the sequence provided as SEQ ID NO: 9705.

(SEQ ID NO: 9705)
ACTCTTAAGAAGGGACGGGGCCCCACGCTGCGCACCCGCGGGTTTGCTAT
GGCGATGAGCAGCGGCGGCAGTGGTGGCGGCGTCCCGGAGCAGGAGGATT
CCGTGCTGTTCCGGCGCGGCACAGGCCAGAGCGATGATTCTGACATTTGG
GATGATACAGCACTGATAAAAGCATATGATAAAGCTGTGGCTTCATTTAA
GCATGCTCTAAAGAATGGTGACATTTGTGAAACTTCGGGTAAACCAAAAA
CCACACCTAAAAGAAAACCTGCTAAGAAGAATAAAAGCCAAAAGAAGAAT
ACTGCAGCTTCCTTACAACAGAATGAAAATGAAAGCCAAGTTTCAACAGA
TGAAAGTGAGAACTCCAGGTCTCCTGGAAATAAATCAGATAACATCAAGC
CCAAATCTGCTCCATGGAACTCTTTTCTCCCTCCACCACCCCCCATGCCA
GGGCCAAGACTGGGACCAGGAAAGCCAGGTCTAAAATTCAATGGCCCACC
ACCGCCACCGCCACCACCACCACCCCACTTACTATCATGCTGGCTGCCTC
CATTTCCTTCTGGACCACCAATAATTCCCCCACCACCTCCCATATGTCCA
GATTCTCTTGATGATGCTGATGCTTTGGGAAGTATGTTAATTTCATGGTA
CATGAGTGGCTATCATACTGGCTATTATATGGAAATGCTGGCATAGAGCA
GCACTAAATGACACCACTAAAGAAACGATCAGACAGATCTGGAATGTGAA
GCGTTATAGAAGATAACTGGCCTCATTTCTTCAAAATATCAAGTGTTGGG
AAAGAAAAAAGGAAGTGGAATGGGTAACTCTTCTTGATTAAAAGTTATGT
AATAACCAAATGCAATGTGAAATATTTTACTGGACTCTATTTTGAAAAAC
CATCTGTAAAAGACTGAGGTGGGGGGGGAGGCCAGCACGGTGGTGAGGCA
GTTGAGAAAATTTGAATGTGGATTAGATTTTGAATGATATTGGATAATTA
TTGGTAATTTTATGAGCTGTGAGAAGGGTGTTGTAGTTTATAAAAGACTG
TCTTAATTTGCATACTTAAGCATTTAGGAATGAAGTGTTAGAGTGTCTTA
AAATGTTTCAAATGGTTTAACAAAATGTATGTGAGGCGTATGTGGCAAAA
TGTTACAGAATCTAACTGGTGGACATGGCTGTTCATTGTACTGTTTTTTT
CTATCTTCTATATGTTTAAAAGTATATAATAAAAATATTTAATTTTTTTT
TAAATTA
Source: NCBI Reference Sequence: XM_011543601.1)

In various embodiments, a SMN2 mRNA transcript comprises the sequence provided as SEQ ID NO: 9706.

(SEQ ID NO: 9706)
AAGAAGGGACGGGGCCCCACGCTGCGCACCCGCGGGTTTGCTATGGCGAT
GAGCAGCGGCGGCAGTGGTGGCGGCGTCCCGGAGCAGGAGGATTCCGTGC
TGTTCCGGCGCGGCACAGGCCAGAGCGATGATTCTGACATTTGGGATGAT
ACAGCACTGATAAAAGCATATGATAAAGCTGTGGCTTCATTTAAGCATGC
TCTAAAGAATGGTGACATTTGTGAAACTTCGGGTAAACCAAAAACCACAC
CTAAAAGAAAACCTGCTAAGAAGAATAAAAGCCAAAAGAAGAATACTGCA
GCTTCCTTACAACAGTGGAAAGTTGGGGACAAATGTTCTGCCATTTGGTC
AGAAGACGGTTGCATTTACCCAGCTACCATTGCTTCAATTGATTTTAAGA
GAGAAACCTGTGTTGTGGTTTACACTGGATATGGAAATAGAGAGGAGCAA
AATCTGTCCGATCTACTTTCCCCAATCTGTGAAGTAGCTAATAATATAGA
ACAAAATGCTCAAGAGAATGAAAATGAAAGCCAAGTTTCAACAGATGAAA
GTGAGAACTCCAGGTCTCCTGGAAATAAATCAGATAACATCAAGCCCAAA
TCTGCTCCATGGAACTCTTTTCTCCCTCCACCACCCCCCATGCCAGGGCC
AAGACTGGGACCAGGAAAGCCAGGTCTAAAATTCAATGGCCCACCACCGC
CACCGCCACCACCACCACCCCACTTACTATCATGCTGGCTGCCTCCATTT
CCTTCTGGACCACCAATAATTCCCCCACCACCTCCCATATGTCCAGATTC
TCTTGATGATGCTGATGCTTTGGGAAGTATGTTAATTTCATGGTACATGA
GTGGCTATCATACTGGCTATTATATGACAGGGTTTCACTGTGTTAGCCAG
GATGGTCTCAATCTCCTGACCCCGTGATCCACCCGCCTCGGCCTTCCAAG
AGAAATGAAATTTTTTTAATGCACAAAGATCTGGGGTAATGTGTACCACA
TTGAACCTTGGGGAGTATGGCTTCAAACTTGTCACTTTATACGTTAGTCT
CCTACGGACATGTTCTATTGTATTTTAGTCAGAACATTTAAAAT
(Source: NCBI Reference Sequence: XM_011543599.1)

In various embodiments, a SMN2 mRNA transcript comprises the sequence provided as SEQ ID NO: 9707.

(SEQ ID NO: 9707)
AAGAAGGGACGGGGCCCCACGCTGCGCACCCGCGGGTTTGCTATGGCGAT
GAGCAGCGGCGGCAGTGGTGGCGGCGTCCCGGAGCAGGAGGATTCCGTGC
TGTTCCGGCGCGGCACAGGCCAGAGCGATGATTCTGACATTTGGGATGAT
ACAGCACTGATAAAAGCATATGATAAAGCTGTGGCTTCATTTAAGCATGC
TCTAAAGAATGGTGACATTTGTGAAACTTCGGGTAAACCAAAAACCACAC
CTAAAAGAAAACCTGCTAAGAAGAATAAAAGCCAAAAGAAGAATACTGCA
GCTTCCTTACAACAGTGGAAAGTTGGGGACAAATGTTCTGCCATTTGGTC
AGAAGACGGTTGCATTTACCCAGCTACCATTGCTTCAATTGATTTTAAGA
GAGAAACCTGTGTTGTGGTTTACACTGGATATGGAAATAGAGAGGAGCAA
AATCTGTCCGATCTACTTTCCCCAATCTGTGAAGTAGCTAATAATATAGA
ACAAAATGCTCAAGAGAATGAAAATGAAAGCCAAGTTTCAACAGATGAAA
GTGAGAACTCCAGGTCTCCTGGAAATAAATCAGATAACATCAAGCCCAAA
TCTGCTCCATGGAACTCTTTTCTCCCTCCACCACCCCCCATGCCAGGGCC
AAGACTGGGACCAGGAAAGCCAGGTCTAAAATTCAATGGCCCACCACCGC
CACCGCCACCACCACCACCCCACTTACTATCATGCTGGCTGCCTCCATTT
CCTTCTGGACCACCAATAATTCCCCCACCACCTCCCATATGTCCAGATTC
TCTTGATGATGCTGATGCTTTGGGAAGTATGTTAATTTCATGGTACATGA
GTGGCTATCATACTGGCTATTATATGGGTTTCACTGTGTTAGCCAGGATG
GTCTCAATCTCCTGACCCCGTGATCCACCCGCCTCGGCCTTCCAAGAGAA
ATGAAATTTTTTTAATGCACAAAGATCTGGGGTAATGTGTACCACATTGA
ACCTTGGGGAGTATGGCTTCAAACTTGTCACTTTATACGTTAGTCTCCTA
CGGACATGTTCTATTGTATTTTAGTCAGAACATTTAAAAT
(Source: NCBI Reference Sequence: XM_017009787.1)

In various embodiments, a SMN2 transcript is a SMN2 pre-mRNA transcript. In various embodiments, a SMN2 pre-miRNA transcript comprises a sequence provided as SEQ ID NO: 9708. NCBI Reference Sequence NG_008728.1 is SEQ ID NO: 9708.

In various embodiments, a SMN2 pre-miRNA transcript comprises a sequence provided as SEQ ID NO: 9709. NCBI Reference Sequence NC_000005.10 is SEQ ID NO: 9709.

In various embodiments, the SMN2 transcript shares between 90-100% identity with any one of SEQ ID NO: 9698-9707 or shares between 90-100% identity to a SMN2 pre-mRNA transcript comprising SEQ ID NO: 9708 or SEQ ID NO: 9709. In various embodiments, the SMN2 transcript shares at least 90%, 91%, 92%, 93%, 94%, 95%, 96%, 97%, 98%, or 99% identity with SEQ ID NO: 9698-9707 or shares at least 90%, 91%, 92%, 93%, 94%, 95%, 96%, 97%, 98%, or 99% identity with a SMN2 pre-mRNA transcript transcribed from SEQ ID NO: 9708 or SEQ ID NO: 9709.

### SMN2 Oligonucleotides Targeting Regions of the SMN2 Transcript

In various embodiments, SMN2 AON disclosed herein are complementary to specific regions of SMN2 transcripts (for example, a SMN2 transcript) comprising a sequence that shares at least 90% (e.g., 90%, 91%, 92%, 93%, 94%, 95%, 96%, 97%, 98%, 99%, or 100%) identity to any one of SEQ ID NOs: 9698-9709. In some embodiments, a SMN2 AON comprises a sequence that is complementary to a specific region of the SMN2 transcript comprising a sequence that shares at least 90% (e.g., 90%, 91%, 92%, 93%, 94%, 95%, 96%, 97%, 98%, 99%, or 100%) identity to any one of SEQ ID NOs: 9698-9709. In some embodiments, a SMN2 AON comprises a sequence that is at least 85% complementary to a specific region of the SMN2 transcript. In some embodiments, a SMN2 AON comprises a sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% complementary to a specific region of the SMN2 transcript. In some embodiments, a SMN2 AON comprises a sequence that is 90 to 99% complementary to a specific region of the SMN2 transcript. In some embodiments, a SMN2 AON comprises a sequence that is 90 to 95% complementary to a specific region of the SMN2 transcript. In some embodiments, a SMN2 AON comprises a sequence that is 95 to 99% complementary to a specific region of the SMN2 transcript

In some embodiments, the SMN2 AON (e.g., SMN2 AON) has a segment that has, at most, 7 linked nucleosides. In some embodiments, the SMN2 AON has a segment that has, at most, 6, 5, 4, 3, or 2 linked nucleosides. The segments of the SMN2 AON may be separated from other segments of the SMN2 AON through a spacer. The segment of the SMN2 AON is complementary to a specific region of the SMN2 transcript (for example, a SMN2 transcript) comprising a sequence that shares at least 90% (e.g., 90%, 91%, 92%, 93%, 94%, 95%, 96%, 97%, 98%, 99%, or 100%) identity to any one of SEQ ID NOs: 9698-9709.

### SMN2 Oligonucleotide Variants

In various embodiments, SMN2 AONs include different variants, hereafter referred to as SMN2 AON variants. A SMN2 AON variant may be an oligonucleotide sequence of 5 to 100 nucleobases in length, for example, 10 to 40 nucleobases in length, for example, 14 to 40 nucleobases in length, 10 to 30 nucleobases in length, for example, 14 to 30 nucleobases in length, for example, 16 to 28 nucleobases in length, for example, 19 to 23 nucleobases in length, for example, 21 to 23 nucleobases in length, for example, or 18, 19, 20, 21, 22, 23, 24, 25, 26, 27, 28, 29, or 30 nucleobases in length. A SMN2 AON variant may be an oligonucleotide sequence complementary to a portion of a SMN2 pre-mRNA sequence or a SMN2 gene sequence. Example SMN2 AON variants include a nucleobase sequence selected from any one of SEQ ID NOs:9710-10141.

In various embodiments, a SMN2 AON variant represents a modified version of a corresponding SMN2 parent oligonucleotide. In some embodiments, a SMN2 AON variant includes a nucleobase sequence that represents a shortened version of a nucleobase sequence of a SMN2 parent AON. As one example, if a SMN2 parent oligonucleotide includes a 25mer (e.g., 25 nucleotide bases in length) a variant (e.g., a SMN2 variant) may include a shorter version (e.g., 15mer, 17mer, 19mer, 21mer, or 23mer) of the 25mer SMN2 parent oligonucleotide. In one embodiment, a nucleobase sequence of a SMN2 AON variant differs from a corresponding nucleobase sequence of a SMN2 parent oligonucleotide in that 1, 2, 3, 4, 5, or 6 nucleotide bases are removed from one or both of the 3′ and 5′ ends of the nucleobase sequence of the SMN2 parent oligonucleotide. In one embodiment, the corresponding SMN2 AON variant may include a 23mer where two nucleotide bases were removed from one of the 3′ or 5′ end of a 25mer included in the SMN2 parent oligonucleotide. In one embodiment, the corresponding SMN2 AON variant may include a 23mer where one nucleotide base is removed from each of the 3′ and 5′ ends of the 25mer included in the SMN2 parent oligonucleotide. In one embodiment, the corresponding SMN2 AON variant may include a 21mer where two nucleotide bases are removed from each of the 3′ and 5′ ends of the 25mer included in the SMN2 parent oligonucleotide. In one embodiment, the corresponding SMN2 AON variant may include a 21mer where four nucleotide bases are removed from either the 3′ or 5′ end of the 25mer included in the SMN2 parent oligonucleotide. In one embodiment, the corresponding SMN2 AON variant may include a 19mer where three nucleotide bases are removed from each of the 3′ and 5′ ends of the 25mer included in the SMN2 parent oligonucleotide. In one embodiment, the corresponding SMN2 AON variant may include a 19mer where six nucleotide bases are removed from either the 3′ or 5′ end of the 25mer included in the SMN2 parent oligonucleotide.

Example sequences of SMN2 AON variants are shown below in Table 11.

TABLE 11
SMN2 Oligonucleotide Variant Sequences complementary to a sequence a
SMN2 transcript

| SEQ ID | | SEQ ID | |
|---|---|---|---|
| NO: | AON Sequence* (5′ → 3′) | NO: | Target Sequence (5′ → 3′) |
| 9710 | ATCCCAAAGAAAACAATTATTAG | 10142 | CTAATAATTGTTTTCTTTGGGAT |
| 9711 | TATCCCAAAGAAAACAATTATTA | 10143 | TAATAATTGTTTTCTTTGGGATA |
| 9712 | TTATCCCAAAGAAAACAATTATT | 10144 | AATAATTGTTTTCTTTGGGATAA |
| 9713 | GTTATCCCAAAGAAAACAATTAT | 10145 | ATAATTGTTTTCTTTGGGATAAC |
| 9714 | AGTTATCCCAAAGAAAACAATTA | 10146 | TAATTGTTTTCTTTGGGATAACT |
| 9715 | AAGTTATCCCAAAGAAAACAATT | 10147 | AATTGTTTTCTTTGGGATAACTT |
| 9716 | AAAGTTATCCCAAAGAAAACAAT | 10148 | ATTGTTTTCTTTGGGATAACTTT |
| 9717 | AAAAGTTATCCCAAAGAAAACAA | 10149 | TTGTTTTCTTTGGGATAACTTTT |
| 9718 | TAAAAGTTATCCCAAAGAAAACA | 10150 | TGTTTTCTTTGGGATAACTTTTA |
| 9719 | TTAAAAGTTATCCCAAAGAAAAC | 10151 | GTTTTCTTTGGGATAACTTTTAA |
| 9720 | TTTAAAAGTTATCCCAAAGAAAA | 10152 | TTTTCTTTGGGATAACTTTTAAA |
| 9721 | CTTTAAAAGTTATCCCAAAGAAA | 10153 | TTTCTTTGGGATAACTTTTAAAG |
| 9722 | ACTTTAAAAGTTATCCCAAAGAA | 10154 | TTCTTTGGGATAACTTTTAAAGT |
| 9723 | TACTTTAAAAGTTATCCCAAAGA | 10155 | TCTTTGGGATAACTTTTAAAGTA |
| 9724 | GTACTTTAAAAGTTATCCCAAAG | 10156 | CTTTGGGATAACTTTTAAAGTAC |
| 9725 | TGTACTTTAAAAGTTATCCCAAA | 10157 | TTTGGGATAACTTTTAAAGTACA |
| 9726 | ATGTACTTTAAAAGTTATCCCAA | 10158 | TTGGGATAACTTTTAAAGTACAT |
| 9727 | AATGTACTTTAAAAGTTATCCCA | 10159 | TGGGATAACTTTTAAAGTACATT |
| 9728 | TAATGTACTTTAAAAGTTATCCC | 10160 | GGGATAACTTTTAAAGTACATTA |
| 9729 | TTAATGTACTTTAAAAGTTATCC | 10161 | GGATAACTTTTAAAGTACATTAA |
| 9730 | TTTAATGTACTTTAAAAGTTATC | 10162 | GATAACTTTTAAAGTACATTAAA |
| 9731 | TTTTAATGTACTTTAAAAGTTAT | 10163 | ATAACTTTTAAAGTACATTAAAA |
| 9732 | CTTTTAATGTACTTTAAAAGTTA | 10164 | TAACTTTTAAAGTACATTAAAAG |
| 9733 | TCTTTTAATGTACTTTAAAAGTT | 10165 | AACTTTTAAAGTACATTAAAAGA |
| 9734 | GTCTTTTAATGTACTTTAAAAGT | 10166 | ACTTTTAAAGTACATTAAAAGAC |
| 9735 | AGTCTTTTAATGTACTTTAAAAG | 10167 | CTTTTAAAGTACATTAAAAGACT |
| 9736 | TAGTCTTTTAATGTACTTTAAAA | 10168 | TTTTAAAGTACATTAAAAGACTA |
| 9737 | ATAGTCTTTTAATGTACTTTAAA | 10169 | TTTAAAGTACATTAAAAGACTAT |
| 9738 | GATAGTCTTTTAATGTACTTTAA | 10170 | TTAAAGTACATTAAAAGACTATC |
| 9739 | TGATAGTCTTTTAATGTACTTTA | 10171 | TAAAGTACATTAAAAGACTATCA |
| 9740 | TTGATAGTCTTTTAATGTACTTT | 10172 | AAAGTACATTAAAAGACTATCAA |
| 9741 | GTTGATAGTCTTTTAATGTACTT | 10173 | AAGTACATTAAAAGACTATCAAC |
| 9742 | AGTTGATAGTCTTTTAATGTACT | 10174 | AGTACATTAAAAGACTATCAACT |
| 9743 | AAGTTGATAGTCTTTTAATGTAC | 10175 | GTACATTAAAAGACTATCAACTT |
| 9744 | TAAGTTGATAGTCTTTTAATGTA | 10176 | TACATTAAAAGACTATCAACTTA |
| 9745 | TTAAGTTGATAGTCTTTTAATGT | 10177 | ACATTAAAAGACTATCAACTTAA |
| 9746 | ATTAAGTTGATAGTCTTTTAATG | 10178 | CATTAAAAGACTATCAACTTAAT |
| 9747 | AATTAAGTTGATAGTCTTTTAAT | 10179 | ATTAAAAGACTATCAACTTAATT |
| 9748 | AAATTAAGTTGATAGTCTTTTAA | 10180 | TTAAAAGACTATCAACTTAATTT |
| 9749 | GAAATTAAGTTGATAGTCTTTTA | 10181 | TAAAAGACTATCAACTTAATTTC |
| 9750 | AGAAATTAAGTTGATAGTCTTTT | 10182 | AAAAGACTATCAACTTAATTTCT |
| 9751 | CAGAAATTAAGTTGATAGTCTTT | 10183 | AAAGACTATCAACTTAATTTCTG |
| 9752 | TCAGAAATTAAGTTGATAGTCTT | 10184 | AAGACTATCAACTTAATTTCTGA |
| 9753 | ATCAGAAATTAAGTTGATAGTCT | 10185 | AGACTATCAACTTAATTTCTGAT |
| 9754 | GATCAGAAATTAAGTTGATAGTC | 10186 | GACTATCAACTTAATTTCTGATC |
| 9755 | TGATCAGAAATTAAGTTGATAGT | 10187 | ACTATCAACTTAATTTCTGATCA |
| 9756 | ATGATCAGAAATTAAGTTGATAG | 10188 | CTATCAACTTAATTTCTGATCAT |
| 9757 | TATGATCAGAAATTAAGTTGATA | 10189 | TATCAACTTAATTTCTGATCATA |
| 9758 | ATATGATCAGAAATTAAGTTGAT | 10190 | ATCAACTTAATTTCTGATCATAT |
| 9759 | AATATGATCAGAAATTAAGTTGA | 10191 | TCAACTTAATTTCTGATCATATT |
| 9760 | AAATATGATCAGAAATTAAGTTG | 10192 | CAACTTAATTTCTGATCATATTT |
| 9761 | AAAATATGATCAGAAATTAAGTT | 10193 | AACTTAATTTCTGATCATATTTT |
| 9762 | CAAAATATGATCAGAAATTAAGT | 10194 | ACTTAATTTCTGATCATATTTTG |
| 9763 | ACAAAATATGATCAGAAATTAAG | 10195 | CTTAATTTCTGATCATATTTTGT |
| 9764 | AACAAAATATGATCAGAAATTAA | 10196 | TTAATTTCTGATCATATTTTGTT |
| 9765 | CAACAAAATATGATCAGAAATTA | 10197 | TAATTTCTGATCATATTTTGTTG |
| 9766 | TCAACAAAATATGATCAGAAATT | 10198 | AATTTCTGATCATATTTTGTTGA |
| 9767 | TTCAACAAAATATGATCAGAAAT | 10199 | ATTTCTGATCATATTTTGTTGAA |
| 9768 | ATTCAACAAAATATGATCAGAAA | 10200 | TTTCTGATCATATTTTGTTGAAT |
| 9769 | TATTCAACAAAATATGATCAGAA | 10201 | TTCTGATCATATTTTGTTGAATA |
| 9770 | TTATTCAACAAAATATGATCAGA | 10202 | TCTGATCATATTTTGTTGAATAA |
| 9771 | TTTATTCAACAAAATATGATCAG | 10203 | CTGATCATATTTTGTTGAATAAA |
| 9772 | TTTTATTCAACAAAATATGATCA | 10204 | TGATCATATTTTGTTGAATAAAA |
| 9773 | ATTTTATTCAACAAAATATGATC | 10205 | GATCATATTTTGTTGAATAAAAT |
| 9774 | TATTTTATTCAACAAAATATGAT | 10206 | ATCATATTTTGTTGAATAAAATA |
| 9775 | TTATTTTATTCAACAAAATATGA | 10207 | TCATATTTTGTTGAATAAAATAA |
| 9776 | CTTATTTTATTCAACAAAATATG | 10208 | CATATTTTGTTGAATAAAATAAG |
| 9777 | ACTTATTTTATTCAACAAAATAT | 10209 | ATATTTTGTTGAATAAAATAAGT |
| 9778 | TACTTATTTTATTCAACAAAATA | 10210 | TATTTTGTTGAATAAAATAAGTA |
| 9779 | TTACTTATTTTATTCAACAAAAT | 10211 | ATTTTGTTGAATAAAATAAGTAA |
| 9780 | TTTACTTATTTTATTCAACAAAA | 10212 | TTTTGTTGAATAAAATAAGTAAA |
| 9781 | TTTTACTTATTTTATTCAACAAA | 10213 | TTTGTTGAATAAAATAAGTAAAA |
| 9782 | ATTTTACTTATTTTATTCAACAA | 10214 | TTGTTGAATAAAATAAGTAAAAT |
| 9783 | CATTTTACTTATTTTATTCAACA | 10215 | TGTTGAATAAAATAAGTAAAATG |
| 9784 | ACATTTTACTTATTTTATTCAAC | 10216 | GTTGAATAAAATAAGTAAAATGT |
| 9785 | GACATTTTACTTATTTTATTCAA | 10217 | TTGAATAAAATAAGTAAAATGTC |
| 9786 | AGACATTTTACTTATTTTATTCA | 10218 | TGAATAAAATAAGTAAAATGTCT |
| 9787 | AAGACATTTTACTTATTTTATTC | 10219 | GAATAAAATAAGTAAAATGTCTT |
| 9788 | CAAGACATTTTACTTATTTTATT | 10220 | AATAAAATAAGTAAAATGTCTTG |
| 9789 | ACAAGACATTTTACTTATTTTAT | 10221 | ATAAAATAAGTAAAATGTCTTGT |
| 9790 | CACAAGACATTTTACTTATTTTA | 10222 | TAAAATAAGTAAAATGTCTTGTG |
| 9791 | TCACAAGACATTTTACTTATTTT | 10223 | AAAATAAGTAAAATGTCTTGTGA |
| 9792 | TTCACAAGACATTTTACTTATTT | 10224 | AAATAAGTAAAATGTCTTGTGAA |
| 9793 | TTTCACAAGACATTTTACTTATT | 10225 | AATAAGTAAAATGTCTTGTGAAA |
| 9794 | GTTTCACAAGACATTTTACTTAT | 10226 | ATAAGTAAAATGTCTTGTGAAAC |
| 9795 | TGTTTCACAAGACATTTTACTTA | 10227 | TAAGTAAAATGTCTTGTGAAACA |
| 9796 | TTGTTTCACAAGACATTTTACTT | 10228 | AAGTAAAATGTCTTGTGAAACAA |
| 9797 | TTTGTTTCACAAGACATTTTACT | 10229 | AGTAAAATGTCTTGTGAAACAAA |
| 9798 | TTTTGTTTCACAAGACATTTTAC | 10230 | GTAAAATGTCTTGTGAAACAAAA |
| 9799 | ATTTTGTTTCACAAGACATTTTA | 10231 | TAAAATGTCTTGTGAAACAAAAT |
| 9800 | CATTTTGTTTCACAAGACATTTT | 10232 | AAAATGTCTTGTGAAACAAAATG |
| 9801 | GCATTTTGTTTCACAAGACATTT | 10233 | AAATGTCTTGTGAAACAAAATGC |
| 9802 | AGCATTTTGTTTCACAAGACATT | 10234 | AATGTCTTGTGAAACAAAATGCT |
| 9803 | AAGCATTTTGTTTCACAAGACAT | 10235 | ATGTCTTGTGAAACAAAATGCTT |
| 9804 | AAAGCATTTTGTTTCACAAGACA | 10236 | TGTCTTGTGAAACAAAATGCTTT |
| 9805 | AAAAGCATTTTGTTTCACAAGAC | 10237 | GTCTTGTGAAACAAAATGCTTTT |
| 9806 | AAAAAGCATTTTGTTTCACAAGA | 10238 | TCTTGTGAAACAAAATGCTTTTT |
| 9807 | TAAAAAGCATTTTGTTTCACAAG | 10239 | CTTGTGAAACAAAATGCTTTTTA |
| 9808 | TTAAAAAGCATTTTGTTTCACAA | 10240 | TTGTGAAACAAAATGCTTTTTAA |
| 9809 | GTTAAAAAGCATTTTGTTTCACA | 10241 | TGTGAAACAAAATGCTTTTTAAC |
| 9810 | TGTTAAAAAGCATTTTGTTTCAC | 10242 | GTGAAACAAAATGCTTTTTAACA |
| 9811 | ATGTTAAAAAGCATTTTGTTTCA | 10243 | TGAAACAAAATGCTTTTTAACAT |
| 9812 | GATGTTAAAAAGCATTTTGTTTC | 10244 | GAAACAAAATGCTTTTTAACATC |
| 9813 | GGATGTTAAAAAGCATTTTGTTT | 10245 | AAACAAAATGCTTTTTAACATCC |
| 9814 | TGGATGTTAAAAAGCATTTTGTT | 10246 | AACAAAATGCTTTTTAACATCCA |
| 9815 | ATGGATGTTAAAAAGCATTTTGT | 10247 | ACAAAATGCTTTTTAACATCCAT |
| 9816 | TATGGATGTTAAAAAGCATTTTG | 10248 | CAAAATGCTTTTTAACATCCATA |
| 9817 | ATATGGATGTTAAAAAGCATTTT | 10249 | AAAATGCTTTTTAACATCCATAT |
| 9818 | TATATGGATGTTAAAAAGCATTT | 10250 | AAATGCTTTTTAACATCCATATA |
| 9819 | TTATATGGATGTTAAAAAGCATT | 10251 | AATGCTTTTTAACATCCATATAA |
| 9820 | TTTATATGGATGTTAAAAAGCAT | 10252 | ATGCTTTTTAACATCCATATAAA |
| 9821 | CTTTATATGGATGTTAAAAAGCA | 10253 | TGCTTTTTAACATCCATATAAAG |
| 9822 | GCTTTATATGGATGTTAAAAAGC | 10254 | GCTTTTTAACATCCATATAAAGC |
| 9823 | AGCTTTATATGGATGTTAAAAAG | 10255 | CTTTTTAACATCCATATAAAGCT |
| 9824 | TAGCTTTATATGGATGTTAAAAA | 10256 | TTTTTAACATCCATATAAAGCTA |
| 9825 | ATAGCTTTATATGGATGTTAAAA | 10257 | TTTTAACATCCATATAAAGCTAT |
| 9826 | GATAGCTTTATATGGATGTTAAA | 10258 | TTTAACATCCATATAAAGCTATC |
| 9827 | AGATAGCTTTATATGGATGTTAA | 10259 | TTAACATCCATATAAAGCTATCT |
| 9828 | TAGATAGCTTTATATGGATGTTA | 10260 | TAACATCCATATAAAGCTATCTA |
| 9829 | ATAGATAGCTTTATATGGATGTT | 10261 | AACATCCATATAAAGCTATCTAT |
| 9830 | TATAGATAGCTTTATATGGATGT | 10262 | ACATCCATATAAAGCTATCTATA |
| 9831 | ATATAGATAGCTTTATATGGATG | 10263 | CATCCATATAAAGCTATCTATAT |
| 9832 | TATATAGATAGCTTTATATGGAT | 10264 | ATCCATATAAAGCTATCTATATA |
| 9833 | ATATATAGATAGCTTTATATGGA | 10265 | TCCATATAAAGCTATCTATATAT |
| 9834 | TATATATAGATAGCTTTATATGG | 10266 | CCATATAAAGCTATCTATATATA |
| 9835 | CTATATATAGATAGCTTTATATG | 10267 | CATATAAAGCTATCTATATATAG |
| 9836 | GCTATATATAGATAGCTTTATAT | 10268 | ATATAAAGCTATCTATATATAGC |
| 9837 | AGCTATATATAGATAGCTTTATA | 10269 | TATAAAGCTATCTATATATAGCT |
| 9838 | TAGCTATATATAGATAGCTTTAT | 10270 | ATAAAGCTATCTATATATAGCTA |
| 9839 | ATAGCTATATATAGATAGCTTTA | 10271 | TAAAGCTATCTATATATAGCTAT |
| 9840 | GATAGCTATATATAGATAGCTTT | 10272 | AAAGCTATCTATATATAGCTATC |
| 9841 | AGATAGCTATATATAGATAGCTT | 10273 | AAGCTATCTATATATAGCTATCT |
| 9842 | TAGATAGCTATATATAGATAGCT | 10274 | AGCTATCTATATATAGCTATCTA |
| 9843 | ATAGATAGCTATATATAGATAGC | 10275 | GCTATCTATATATAGCTATCTAT |
| 9844 | TATAGATAGCTATATATAGATAG | 10276 | CTATCTATATATAGCTATCTATA |
| 9845 | ATATAGATAGCTATATATAGATA | 10277 | TATCTATATATAGCTATCTATAT |
| 9846 | GATATAGATAGCTATATATAGAT | 10278 | ATCTATATATAGCTATCTATATC |
| 9847 | AGATATAGATAGCTATATATAGA | 10279 | TCTATATATAGCTATCTATATCT |
| 9848 | TAGATATAGATAGCTATATATAG | 10280 | CTATATATAGCTATCTATATCTA |
| 9849 | ATAGATATAGATAGCTATATATA | 10281 | TATATATAGCTATCTATATCTAT |
| 9850 | TATAGATATAGATAGCTATATAT | 10282 | ATATATAGCTATCTATATCTATA |
| 9851 | ATATAGATATAGATAGCTATATA | 10283 | TATATAGCTATCTATATCTATAT |
| 9852 | TATATAGATATAGATAGCTATAT | 10284 | ATATAGCTATCTATATCTATATA |
| 9853 | CTATATAGATATAGATAGCTATA | 10285 | TATAGCTATCTATATCTATATAG |
| 9854 | GCTATATAGATATAGATAGCTAT | 10286 | ATAGCTATCTATATCTATATAGC |
| 9855 | AGCTATATAGATATAGATAGCTA | 10287 | TAGCTATCTATATCTATATAGCT |
| 9856 | TAGCTATATAGATATAGATAGCT | 10288 | AGCTATCTATATCTATATAGCTA |
| 9857 | ATAGCTATATAGATATAGATAGC | 10289 | GCTATCTATATCTATATAGCTAT |
| 9858 | AATAGCTATATAGATATAGATAG | 10290 | CTATCTATATCTATATAGCTATT |
| 9859 | AAATAGCTATATAGATATAGATA | 10291 | TATCTATATCTATATAGCTATTT |
| 9860 | AAAATAGCTATATAGATATAGAT | 10292 | ATCTATATCTATATAGCTATTTT |
| 9861 | AAAAATAGCTATATAGATATAGA | 10293 | TCTATATCTATATAGCTATTTTT |
| 9862 | AAAAAATAGCTATATAGATATAG | 10294 | CTATATCTATATAGCTATTTTTT |
| 9863 | AAAAAAATAGCTATATAGATATA | 10295 | TATATCTATATAGCTATTTTTTT |
| 9864 | AAAAAAAATAGCTATATAGATAT | 10296 | ATATCTATATAGCTATTTTTTTT |
| 9865 | TAAAAAAAATAGCTATATAGATA | 10297 | TATCTATATAGCTATTTTTTTTA |
| 9866 | TTAAAAAAAATAGCTATATAGAT | 10298 | ATCTATATAGCTATTTTTTTTAA |
| 9867 | GTTAAAAAAAATAGCTATATAGA | 10299 | TCTATATAGCTATTTTTTTTAAC |
| 9868 | AGTTAAAAAAAATAGCTATATAG | 10300 | CTATATAGCTATTTTTTTTAACT |
| 9869 | AAGTTAAAAAAAATAGCTATATA | 10301 | TATATAGCTATTTTTTTTAACTT |
| 9870 | GAAGTTAAAAAAAATAGCTATAT | 10302 | ATATAGCTATTTTTTTTAACTTC |
| 9871 | GGAAGTTAAAAAAAATAGCTATA | 10303 | TATAGCTATTTTTTTTAACTTCC |
| 9872 | AGGAAGTTAAAAAAAATAGCTAT | 10304 | ATAGCTATTTTTTTTAACTTCCT |
| 9873 | AAGGAAGTTAAAAAAAATAGCTA | 10305 | TAGCTATTTTTTTTAACTTCCTT |
| 9874 | AAAGGAAGTTAAAAAAAATAGCT | 10306 | AGCTATTTTTTTTAACTTCCTTT |
| 9875 | TAAAGGAAGTTAAAAAAAATAGC | 10307 | GCTATTTTTTTTAACTTCCTTTA |
| 9876 | ATAAAGGAAGTTAAAAAAAATAG | 10308 | CTATTTTTTTTAACTTCCTTTAT |
| 9877 | AATAAAGGAAGTTAAAAAAAATA | 10309 | TATTTTTTTTAACTTCCTTTATT |
| 9878 | AAATAAAGGAAGTTAAAAAAAAT | 10310 | ATTTTTTTTAACTTCCTTTATTT |
| 9879 | AAAATAAAGGAAGTTAAAAAAAA | 10311 | TTTTTTTTAACTTCCTTTATTTT |
| 9880 | GAAAATAAAGGAAGTTAAAAAAA | 10312 | TTTTTTTAACTTCCTTTATTTTC |
| 9881 | GGAAAATAAAGGAAGTTAAAAAA | 10313 | TTTTTTAACTTCCTTTATTTTCC |
| 9882 | AGGAAAATAAAGGAAGTTAAAAA | 10314 | TTTTTAACTTCCTTTATTTTCCT |
| 9883 | AAGGAAAATAAAGGAAGTTAAAA | 10315 | TTTTAACTTCCTTTATTTTCCTT |
| 9884 | TAAGGAAAATAAAGGAAGTTAAA | 10316 | TTTAACTTCCTTTATTTTCCTTA |
| 9885 | GTAAGGAAAATAAAGGAAGTTAA | 10317 | TTAACTTCCTTTATTTTCCTTAC |
| 9886 | TGTAAGGAAAATAAAGGAAGTTA | 10318 | TAACTTCCTTTATTTTCCTTACA |
| 9887 | CTGTAAGGAAAATAAAGGAAGTT | 10319 | AACTTCCTTTATTTTCCTTACAG |
| 9888 | CCTGTAAGGAAAATAAAGGAAGT | 10320 | ACTTCCTTTATTTTCCTTACAGG |
| 9889 | CCCTGTAAGGAAAATAAAGGAAG | 10321 | CTTCCTTTATTTTCCTTACAGGG |
| 9890 | ACCCTGTAAGGAAAATAAAGGAA | 10322 | TTCCTTTATTTTCCTTACAGGGT |
| 9891 | AACCCTGTAAGGAAAATAAAGGA | 10323 | TCCTTTATTTTCCTTACAGGGTT |
| 9892 | AAACCCTGTAAGGAAAATAAAGG | 10324 | CCTTTATTTTCCTTACAGGGTTT |
| 9893 | AAAACCCTGTAAGGAAAATAAAG | 10325 | CTTTATTTTCCTTACAGGGTTTT |
| 9894 | TAAAACCCTGTAAGGAAAATAAA | 10326 | TTTATTTTCCTTACAGGGTTTTA |
| 9895 | CTAAAACCCTGTAAGGAAAATAA | 10327 | TTATTTTCCTTACAGGGTTTTAG |
| 9896 | TCTAAAACCCTGTAAGGAAAATA | 10328 | TATTTTCCTTACAGGGTTTTAGA |
| 9897 | GTCTAAAACCCTGTAAGGAAAAT | 10329 | ATTTTCCTTACAGGGTTTTAGAC |
| 9898 | TGTCTAAAACCCTGTAAGGAAAA | 10330 | TTTTCCTTACAGGGTTTTAGACA |
| 9899 | TTGTCTAAAACCCTGTAAGGAAA | 10331 | TTTCCTTACAGGGTTTTAGACAA |
| 9900 | TTTGTCTAAAACCCTGTAAGGAA | 10332 | TTCCTTACAGGGTTTTAGACAAA |
| 9901 | TTTTGTCTAAAACCCTGTAAGGA | 10333 | TCCTTACAGGGTTTTAGACAAAA |
| 9902 | ATTTTGTCTAAAACCCTGTAAGG | 10334 | CCTTACAGGGTTTTAGACAAAAT |
| 9903 | GATTTTGTCTAAAACCCTGTAAG | 10335 | CTTACAGGGTTTTAGACAAAATC |
| 9904 | TGATTTTGTCTAAAACCCTGTAA | 10336 | TTACAGGGTTTTAGACAAAATCA |
| 9905 | TTGATTTTGTCTAAAACCCTGTA | 10337 | TACAGGGTTTTAGACAAAATCAA |
| 9906 | TTTGATTTTGTCTAAAACCCTGT | 10338 | ACAGGGTTTTAGACAAAATCAAA |
| 9907 | TTTTGATTTTGTCTAAAACCCTG | 10339 | CAGGGTTTTAGACAAAATCAAAA |
| 9908 | TTTTTGATTTTGTCTAAAACCCT | 10340 | AGGGTTTTAGACAAAATCAAAAA |
| 9909 | CTTTTTGATTTTGTCTAAAACCC | 10341 | GGGTTTTAGACAAAATCAAAAAG |
| 9910 | TCTTTTTGATTTTGTCTAAAACC | 10342 | GGTTTTAGACAAAATCAAAAAGA |
| 9911 | TTCTTTTTGATTTTGTCTAAAAC | 10343 | GTTTTAGACAAAATCAAAAAGAA |
| 9912 | CTTCTTTTTGATTTTGTCTAAAA | 10344 | TTTTAGACAAAATCAAAAAGAAG |
| 9913 | CCTTCTTTTTGATTTTGTCTAAA | 10345 | TTTAGACAAAATCAAAAAGAAGG |
| 9914 | TCCTTCTTTTTGATTTTGTCTAA | 10346 | TTAGACAAAATCAAAAAGAAGGA |
| 9915 | TTCCTTCTTTTTGATTTTGTCTA | 10347 | TAGACAAAATCAAAAAGAAGGAA |
| 9916 | CTTCCTTCTTTTTGATTTTGTCT | 10348 | AGACAAAATCAAAAAGAAGGAAG |
| 9917 | CCTTCCTTCTTTTTGATTTTGTC | 10349 | GACAAAATCAAAAAGAAGGAAGG |
| 9918 | ACCTTCCTTCTTTTTGATTTTGT | 10350 | ACAAAATCAAAAAGAAGGAAGGT |
| 9919 | CACCTTCCTTCTTTTTGATTTTG | 10351 | CAAAATCAAAAAGAAGGAAGGTG |
| 9920 | GCACCTTCCTTCTTTTTGATTTT | 10352 | AAAATCAAAAAGAAGGAAGGTGC |
| 9921 | AGCACCTTCCTTCTTTTTGATTT | 10353 | AAATCAAAAAGAAGGAAGGTGCT |
| 9922 | GAGCACCTTCCTTCTTTTTGATT | 10354 | AATCAAAAAGAAGGAAGGTGCTC |
| 9923 | TGAGCACCTTCCTTCTTTTTGAT | 10355 | ATCAAAAAGAAGGAAGGTGCTCA |
| 9924 | GTGAGCACCTTCCTTCTTTTTGA | 10356 | TCAAAAAGAAGGAAGGTGCTCAC |
| 9925 | TGTGAGCACCTTCCTTCTTTTTG | 10357 | CAAAAAGAAGGAAGGTGCTCACA |
| 9926 | ATGTGAGCACCTTCCTTCTTTTT | 10358 | AAAAAGAAGGAAGGTGCTCACAT |
| 9927 | AATGTGAGCACCTTCCTTCTTTT | 10359 | AAAAGAAGGAAGGTGCTCACATT |
| 9928 | GAATGTGAGCACCTTCCTTCTTT | 10360 | AAAGAAGGAAGGTGCTCACATTC |
| 9929 | GGAATGTGAGCACCTTCCTTCTT | 10361 | AAGAAGGAAGGTGCTCACATTCC |
| 9930 | AGGAATGTGAGCACCTTCCTTCT | 10362 | AGAAGGAAGGTGCTCACATTCCT |
| 9931 | AAGGAATGTGAGCACCTTCCTTC | 10363 | GAAGGAAGGTGCTCACATTCCTT |
| 9932 | TAAGGAATGTGAGCACCTTCCTT | 10364 | AAGGAAGGTGCTCACATTCCTTA |
| 9933 | TTAAGGAATGTGAGCACCTTCCT | 10365 | AGGAAGGTGCTCACATTCCTTAA |
| 9934 | TTTAAGGAATGTGAGCACCTTCC | 10366 | GGAAGGTGCTCACATTCCTTAAA |
| 9935 | ATTTAAGGAATGTGAGCACCTTC | 10367 | GAAGGTGCTCACATTCCTTAAAT |
| 9936 | AATTTAAGGAATGTGAGCACCTT | 10368 | AAGGTGCTCACATTCCTTAAATT |
| 9937 | TAATTTAAGGAATGTGAGCACCT | 10369 | AGGTGCTCACATTCCTTAAATTA |
| 9938 | TTAATTTAAGGAATGTGAGCACC | 10370 | GGTGCTCACATTCCTTAAATTAA |
| 9939 | CTTAATTTAAGGAATGTGAGCAC | 10371 | GTGCTCACATTCCTTAAATTAAG |
| 9940 | CCTTAATTTAAGGAATGTGAGCA | 10372 | TGCTCACATTCCTTAAATTAAGG |
| 9941 | TCCTTAATTTAAGGAATGTGAGC | 10373 | GCTCACATTCCTTAAATTAAGGA |
| 9942 | CTCCTTAATTTAAGGAATGTGAG | 10374 | CTCACATTCCTTAAATTAAGGAG |
| 9943 | ACTCCTTAATTTAAGGAATGTGA | 10375 | TCACATTCCTTAAATTAAGGAGT |
| 9944 | TACTCCTTAATTTAAGGAATGTG | 10376 | CACATTCCTTAAATTAAGGAGTA |
| 9945 | TTACTCCTTAATTTAAGGAATGT | 10377 | ACATTCCTTAAATTAAGGAGTAA |
| 9946 | CTTACTCCTTAATTTAAGGAATG | 10378 | CATTCCTTAAATTAAGGAGTAAG |
| 9947 | ACTTACTCCTTAATTTAAGGAAT | 10379 | ATTCCTTAAATTAAGGAGTAAGT |
| 9948 | GACTTACTCCTTAATTTAAGGAA | 10380 | TTCCTTAAATTAAGGAGTAAGTC |
| 9949 | AGACTTACTCCTTAATTTAAGGA | 10381 | TCCTTAAATTAAGGAGTAAGTCT |
| 9950 | CAGACTTACTCCTTAATTTAAGG | 10382 | CCTTAAATTAAGGAGTAAGTCTG |
| 9951 | GCAGACTTACTCCTTAATTTAAG | 10383 | CTTAAATTAAGGAGTAAGTCTGC |
| 9952 | GGCAGACTTACTCCTTAATTTAA | 10384 | TTAAATTAAGGAGTAAGTCTGCC |
| 9953 | TGGCAGACTTACTCCTTAATTTA | 10385 | TAAATTAAGGAGTAAGTCTGCCA |
| 9954 | CTGGCAGACTTACTCCTTAATTT | 10386 | AAATTAAGGAGTAAGTCTGCCAG |
| 9955 | GCTGGCAGACTTACTCCTTAATT | 10387 | AATTAAGGAGTAAGTCTGCCAGC |
| 9956 | TGCTGGCAGACTTACTCCTTAAT | 10388 | ATTAAGGAGTAAGTCTGCCAGCA |
| 9957 | ATGCTGGCAGACTTACTCCTTAA | 10389 | TTAAGGAGTAAGTCTGCCAGCAT |
| 9958 | AATGCTGGCAGACTTACTCCTTA | 10390 | TAAGGAGTAAGTCTGCCAGCATT |
| 9959 | TAATGCTGGCAGACTTACTCCTT | 10391 | AAGGAGTAAGTCTGCCAGCATTA |
| 9960 | ATAATGCTGGCAGACTTACTCCT | 10392 | AGGAGTAAGTCTGCCAGCATTAT |
| 9961 | CATAATGCTGGCAGACTTACTCC | 10393 | GGAGTAAGTCTGCCAGCATTATG |
| 9962 | TCATAATGCTGGCAGACTTACTC | 10394 | GAGTAAGTCTGCCAGCATTATGA |
| 9963 | TTCATAATGCTGGCAGACTTACT | 10395 | AGTAAGTCTGCCAGCATTATGAA |
| 9964 | TTTCATAATGCTGGCAGACTTAC | 10396 | GTAAGTCTGCCAGCATTATGAAA |
| 9965 | CTTTCATAATGCTGGCAGACTTA | 10397 | TAAGTCTGCCAGCATTATGAAAG |
| 9966 | ACTTTCATAATGCTGGCAGACTT | 10398 | AAGTCTGCCAGCATTATGAAAGT |
| 9967 | CACTTTCATAATGCTGGCAGACT | 10399 | AGTCTGCCAGCATTATGAAAGTG |
| 9968 | TCACTTTCATAATGCTGGCAGAC | 10400 | GTCTGCCAGCATTATGAAAGTGA |
| 9969 | TTCACTTTCATAATGCTGGCAGA | 10401 | TCTGCCAGCATTATGAAAGTGAA |
| 9970 | ATTCACTTTCATAATGCTGGCAG | 10402 | CTGCCAGCATTATGAAAGTGAAT |
| 9971 | GATTCACTTTCATAATGCTGGCA | 10403 | TGCCAGCATTATGAAAGTGAATC |
| 9972 | AGATTCACTTTCATAATGCTGGC | 10404 | GCCAGCATTATGAAAGTGAATCT |
| 9973 | AAGATTCACTTTCATAATGCTGG | 10405 | CCAGCATTATGAAAGTGAATCTT |
| 9974 | TAAGATTCACTTTCATAATGCTG | 10406 | CAGCATTATGAAAGTGAATCTTA |
| 9975 | GTAAGATTCACTTTCATAATGCT | 10407 | AGCATTATGAAAGTGAATCTTAC |
| 9976 | AGTAAGATTCACTTTCATAATGC | 10408 | GCATTATGAAAGTGAATCTTACT |
| 9977 | AAGTAAGATTCACTTTCATAATG | 10409 | CATTATGAAAGTGAATCTTACTT |
| 9978 | AAAGTAAGATTCACTTTCATAAT | 10410 | ATTATGAAAGTGAATCTTACTTT |
| 9979 | AAAAGTAAGATTCACTTTCATAA | 10411 | TTATGAAAGTGAATCTTACTTTT |
| 9980 | CAAAAGTAAGATTCACTTTCATA | 10412 | TATGAAAGTGAATCTTACTTTTG |
| 9981 | ACAAAAGTAAGATTCACTTTCAT | 10413 | ATGAAAGTGAATCTTACTTTTGT |
| 9982 | TACAAAAGTAAGATTCACTTTCA | 10414 | TGAAAGTGAATCTTACTTTTGTA |
| 9983 | TTACAAAAGTAAGATTCACTTTC | 10415 | GAAAGTGAATCTTACTTTTGTAA |
| 9984 | TTTACAAAAGTAAGATTCACTTT | 10416 | AAAGTGAATCTTACTTTTGTAAA |
| 9985 | TTTTACAAAAGTAAGATTCACTT | 10417 | AAGTGAATCTTACTTTTGTAAAA |
| 9986 | GTTTTACAAAAGTAAGATTCACT | 10418 | AGTGAATCTTACTTTTGTAAAAC |
| 9987 | AGTTTTACAAAAGTAAGATTCAC | 10419 | GTGAATCTTACTTTTGTAAAACT |
| 9988 | AAGTTTTACAAAAGTAAGATTCA | 10420 | TGAATCTTACTTTTGTAAAACTT |
| 9989 | AAAGTTTTACAAAAGTAAGATTC | 10421 | GAATCTTACTTTTGTAAAACTTT |
| 9990 | TAAAGTTTTACAAAAGTAAGATT | 10422 | AATCTTACTTTTGTAAAACTTTA |
| 9991 | ATAAAGTTTTACAAAAGTAAGAT | 10423 | ATCTTACTTTTGTAAAACTTTAT |
| 9992 | CATAAAGTTTTACAAAAGTAAGA | 10424 | TCTTACTTTTGTAAAACTTTATG |
| 9993 | CCATAAAGTTTTACAAAAGTAAG | 10425 | CTTACTTTTGTAAAACTTTATGG |
| 9994 | ACCATAAAGTTTTACAAAAGTAA | 10426 | TTACTTTTGTAAAACTTTATGGT |
| 9995 | AACCATAAAGTTTTACAAAAGTA | 10427 | TACTTTTGTAAAACTTTATGGTT |
| 9996 | AAACCATAAAGTTTTACAAAAGT | 10428 | ACTTTTGTAAAACTTTATGGTTT |
| 9997 | CAAACCATAAAGTTTTACAAAAG | 10429 | CTTTTGTAAAACTTTATGGTTTG |
| 9998 | ACAAACCATAAAGTTTTACAAAA | 10430 | TTTTGTAAAACTTTATGGTTTGT |
| 9999 | CACAAACCATAAAGTTTTACAAA | 10431 | TTTGTAAAACTTTATGGTTTGTG |
| 10000 | CCACAAACCATAAAGTTTTACAA | 10432 | TTGTAAAACTTTATGGTTTGTGG |
| 10001 | TCCACAAACCATAAAGTTTTACA | 10433 | TGTAAAACTTTATGGTTTGTGGA |
| 10002 | TTCCACAAACCATAAAGTTTTAC | 10434 | GTAAAACTTTATGGTTTGTGGAA |
| 10003 | TTTCCACAAACCATAAAGTTTTA | 10435 | TAAAACTTTATGGTTTGTGGAAA |
| 10004 | TTTTCCACAAACCATAAAGTTTT | 10436 | AAAACTTTATGGTTTGTGGAAAA |
| 10005 | GTTTTCCACAAACCATAAAGTTT | 10437 | AAACTTTATGGTTTGTGGAAAAC |
| 10006 | TGTTTTCCACAAACCATAAAGTT | 10438 | AACTTTATGGTTTGTGGAAAACA |
| 10007 | TTGTTTTCCACAAACCATAAAGT | 10439 | ACTTTATGGTTTGTGGAAAACAA |
| 10008 | TTTGTTTTCCACAAACCATAAAG | 10440 | CTTTATGGTTTGTGGAAAACAAA |
| 10009 | ATTTGTTTTCCACAAACCATAAA | 10441 | TTTATGGTTTGTGGAAAACAAAT |
| 10010 | CATTTGTTTTCCACAAACCATAA | 10442 | TTATGGTTTGTGGAAAACAAATG |
| 10011 | ACATTTGTTTTCCACAAACCATA | 10443 | TATGGTTTGTGGAAAACAAATGT |
| 10012 | AACATTTGTTTTCCACAAACCAT | 10444 | ATGGTTTGTGGAAAACAAATGTT |
| 10013 | AAACATTTGTTTTCCACAAACCA | 10445 | TGGTTTGTGGAAAACAAATGTTT |
| 10014 | AAAACATTTGTTTTCCACAAACC | 10446 | GGTTTGTGGAAAACAAATGTTTT |
| 10015 | AAAAACATTTGTTTTCCACAAAC | 10447 | GTTTGTGGAAAACAAATGTTTTT |
| 10016 | CAAAAACATTTGTTTTCCACAAA | 10448 | TTTGTGGAAAACAAATGTTTTTG |
| 10017 | TCAAAAACATTTGTTTTCCACAA | 10449 | TTGTGGAAAACAAATGTTTTTGA |
| 10018 | TTCAAAAACATTTGTTTTCCACA | 10450 | TGTGGAAAACAAATGTTTTTGAA |
| 10019 | GTTCAAAAACATTTGTTTTCCAC | 10451 | GTGGAAAACAAATGTTTTTGAAC |
| 10020 | TGTTCAAAAACATTTGTTTTCCA | 10452 | TGGAAAACAAATGTTTTTGAACA |
| 10021 | ATGTTCAAAAACATTTGTTTTCC | 10453 | GGAAAACAAATGTTTTTGAACAT |
| 10022 | AATGTTCAAAAACATTTGTTTTC | 10454 | GAAAACAAATGTTTTTGAACATT |
| 10023 | AAATGTTCAAAAACATTTGTTTT | 10455 | AAAACAAATGTTTTTGAACATTT |
| 10024 | TAAATGTTCAAAAACATTTGTTT | 10456 | AAACAAATGTTTTTGAACATTTA |
| 10025 | TTAAATGTTCAAAAACATTTGTT | 10457 | AACAAATGTTTTTGAACATTTAA |
| 10026 | TTTAAATGTTCAAAAACATTTGT | 10458 | ACAAATGTTTTTGAACATTTAAA |
| 10027 | TTTTAAATGTTCAAAAACATTTG | 10459 | CAAATGTTTTTGAACATTTAAAA |
| 10028 | TTTTTAAATGTTCAAAAACATTT | 10460 | AAATGTTTTTGAACATTTAAAAA |
| 10029 | CTTTTTAAATGTTCAAAAACATT | 10461 | AATGTTTTTGAACATTTAAAAAG |
| 10030 | ACTTTTTAAATGTTCAAAAACAT | 10462 | ATGTTTTTGAACATTTAAAAAGT |
| 10031 | AACTTTTTAAATGTTCAAAAACA | 10463 | TGTTTTTGAACATTTAAAAAGTT |
| 10032 | GAACTTTTTAAATGTTCAAAAAC | 10464 | GTTTTTGAACATTTAAAAAGTTC |
| 10033 | TGAACTTTTTAAATGTTCAAAAA | 10465 | TTTTTGAACATTTAAAAAGTTCA |
| 10034 | CTGAACTTTTTAAATGTTCAAAA | 10466 | TTTTGAACATTTAAAAAGTTCAG |
| 10035 | TCTGAACTTTTTAAATGTTCAAA | 10467 | TTTGAACATTTAAAAAGTTCAGA |
| 10036 | ATCTGAACTTTTTAAATGTTCAA | 10468 | TTGAACATTTAAAAAGTTCAGAT |
| 10037 | CATCTGAACTTTTTAAATGTTCA | 10469 | TGAACATTTAAAAAGTTCAGATG |
| 10038 | ACATCTGAACTTTTTAAATGTTC | 10470 | GAACATTTAAAAAGTTCAGATGT |
| 10039 | AACATCTGAACTTTTTAAATGTT | 10471 | AACATTTAAAAAGTTCAGATGTT |
| 10040 | TAACATCTGAACTTTTTAAATGT | 10472 | ACATTTAAAAAGTTCAGATGTTA |
| 10041 | CTAACATCTGAACTTTTTAAATG | 10473 | CATTTAAAAAGTTCAGATGTTAG |
| 10042 | TCTAACATCTGAACTTTTTAAAT | 10474 | ATTTAAAAAGTTCAGATGTTAGA |
| 10043 | TTCTAACATCTGAACTTTTTAAA | 10475 | TTTAAAAAGTTCAGATGTTAGAA |
| 10044 | TTTCTAACATCTGAACTTTTTAA | 10476 | TTAAAAAGTTCAGATGTTAGAAA |
| 10045 | CTTTCTAACATCTGAACTTTTTA | 10477 | TAAAAAGTTCAGATGTTAGAAAG |
| 10046 | ACTTTCTAACATCTGAACTTTTT | 10478 | AAAAAGTTCAGATGTTAGAAAGT |
| 10047 | AACTTTCTAACATCTGAACTTTT | 10479 | AAAAGTTCAGATGTTAGAAAGTT |
| 10048 | CAACTTTCTAACATCTGAACTTT | 10480 | AAAGTTCAGATGTTAGAAAGTTG |
| 10049 | TCAACTTTCTAACATCTGAACTT | 10481 | AAGTTCAGATGTTAGAAAGTTGA |
| 10050 | TTCAACTTTCTAACATCTGAACT | 10482 | AGTTCAGATGTTAGAAAGTTGAA |
| 10051 | TTTCAACTTTCTAACATCTGAAC | 10483 | GTTCAGATGTTAGAAAGTTGAAA |
| 10052 | CTTTCAACTTTCTAACATCTGAA | 10484 | TTCAGATGTTAGAAAGTTGAAAG |
| 10053 | CCTTTCAACTTTCTAACATCTGA | 10485 | TCAGATGTTAGAAAGTTGAAAGG |
| 10054 | ACCTTTCAACTTTCTAACATCTG | 10486 | CAGATGTTAGAAAGTTGAAAGGT |
| 10055 | AACCTTTCAACTTTCTAACATCT | 10487 | AGATGTTAGAAAGTTGAAAGGTT |
| 10056 | TAACCTTTCAACTTTCTAACATC | 10488 | GATGTTAGAAAGTTGAAAGGTTA |
| 10057 | TTAACCTTTCAACTTTCTAACAT | 10489 | ATGTTAGAAAGTTGAAAGGTTAA |
| 10058 | ATTAACCTTTCAACTTTCTAACA | 10490 | TGTTAGAAAGTTGAAAGGTTAAT |
| 10059 | CATTAACCTTTCAACTTTCTAAC | 10491 | GTTAGAAAGTTGAAAGGTTAATG |
| 10060 | ACATTAACCTTTCAACTTTCTAA | 10492 | TTAGAAAGTTGAAAGGTTAATGT |
| 10061 | TACATTAACCTTTCAACTTTCTA | 10493 | TAGAAAGTTGAAAGGTTAATGTA |
| 10062 | TTACATTAACCTTTCAACTTTCT | 10494 | AGAAAGTTGAAAGGTTAATGTAA |
| 10063 | TTTACATTAACCTTTCAACTTTC | 10495 | GAAAGTTGAAAGGTTAATGTAAA |
| 10064 | TTTTACATTAACCTTTCAACTTT | 10496 | AAAGTTGAAAGGTTAATGTAAAA |
| 10065 | GTTTTACATTAACCTTTCAACTT | 10497 | AAGTTGAAAGGTTAATGTAAAAC |
| 10066 | TGTTTTACATTAACCTTTCAACT | 10498 | AGTTGAAAGGTTAATGTAAAACA |
| 10067 | TTGTTTTACATTAACCTTTCAAC | 10499 | GTTGAAAGGTTAATGTAAAACAA |
| 10068 | ATTGTTTTACATTAACCTTTCAA | 10500 | TTGAAAGGTTAATGTAAAACAAT |
| 10069 | GATTGTTTTACATTAACCTTTCA | 10501 | TGAAAGGTTAATGTAAAACAATC |
| 10070 | TGATTGTTTTACATTAACCTTTC | 10502 | GAAAGGTTAATGTAAAACAATCA |
| 10071 | TTGATTGTTTTACATTAACCTTT | 10503 | AAAGGTTAATGTAAAACAATCAA |
| 10072 | ATTGATTGTTTTACATTAACCTT | 10504 | AAGGTTAATGTAAAACAATCAAT |
| 10073 | TATTGATTGTTTTACATTAACCT | 10505 | AGGTTAATGTAAAACAATCAATA |
| 10074 | ATATTGATTGTTTTACATTAACC | 10506 | GGTTAATGTAAAACAATCAATAT |
| 10075 | AATATTGATTGTTTTACATTAAC | 10507 | GTTAATGTAAAACAATCAATATT |
| 10076 | TAATATTGATTGTTTTACATTAA | 10508 | TTAATGTAAAACAATCAATATTA |
| 10077 | TTAATATTGATTGTTTTACATTA | 10509 | TAATGTAAAACAATCAATATTAA |
| 10078 | TTTAATATTGATTGTTTTACATT | 10510 | AATGTAAAACAATCAATATTAAA |
| 10079 | CTTTAATATTGATTGTTTTACAT | 10511 | ATGTAAAACAATCAATATTAAAG |
| 10080 | TCTTTAATATTGATTGTTTTACA | 10512 | TGTAAAACAATCAATATTAAAGA |
| 10081 | TTCTTTAATATTGATTGTTTTAC | 10513 | GTAAAACAATCAATATTAAAGAA |
| 10082 | ATTCTTTAATATTGATTGTTTTA | 10514 | TAAAACAATCAATATTAAAGAAT |
| 10083 | AATTCTTTAATATTGATTGTTTT | 10515 | AAAACAATCAATATTAAAGAATT |
| 10084 | AAATTCTTTAATATTGATTGTTT | 10516 | AAACAATCAATATTAAAGAATTT |
| 10085 | AAAATTCTTTAATATTGATTGTT | 10517 | AACAATCAATATTAAAGAATTTT |
| 10086 | CAAAATTCTTTAATATTGATTGT | 10518 | ACAATCAATATTAAAGAATTTTG |
| 10087 | TCAAAATTCTTTAATATTGATTG | 10519 | CAATCAATATTAAAGAATTTTGA |
| 10088 | ATCAAAATTCTTTAATATTGATT | 10520 | AATCAATATTAAAGAATTTTGAT |
| 10089 | CATCAAAATTCTTTAATATTGAT | 10521 | ATCAATATTAAAGAATTTTGATG |
| 10090 | GCATCAAAATTCTTTAATATTGA | 10522 | TCAATATTAAAGAATTTTGATGC |
| 10091 | GGCATCAAAATTCTTTAATATTG | 10523 | CAATATTAAAGAATTTTGATGCC |
| 10092 | TGGCATCAAAATTCTTTAATATT | 10524 | AATATTAAAGAATTTTGATGCCA |
| 10093 | TTGGCATCAAAATTCTTTAATAT | 10525 | ATATTAAAGAATTTTGATGCCAA |
| 10094 | TTTGGCATCAAAATTCTTTAATA | 10526 | TATTAAAGAATTTTGATGCCAAA |
| 10095 | TTTTGGCATCAAAATTCTTTAAT | 10527 | ATTAAAGAATTTTGATGCCAAAA |
| 10096 | GTTTTGGCATCAAAATTCTTTAA | 10528 | TTAAAGAATTTTGATGCCAAAAC |
| 10097 | AGTTTTGGCATCAAAATTCTTTA | 10529 | TAAAGAATTTTGATGCCAAAACT |
| 10098 | TAGTTTTGGCATCAAAATTCTTT | 10530 | AAAGAATTTTGATGCCAAAACTA |
| 10099 | ATAGTTTTGGCATCAAAATTCTT | 10531 | AAGAATTTTGATGCCAAAACTAT |
| 10100 | AATAGTTTTGGCATCAAAATTCT | 10532 | AGAATTTTGATGCCAAAACTATT |
| 10101 | TAATAGTTTTGGCATCAAAATTC | 10533 | GAATTTTGATGCCAAAACTATTA |
| 10102 | CTAATAGTTTTGGCATCAAAATT | 10534 | AATTTTGATGCCAAAACTATTAG |
| 10103 | TCTAATAGTTTTGGCATCAAAAT | 10535 | ATTTTGATGCCAAAACTATTAGA |
| 10104 | ATCTAATAGTTTTGGCATCAAAA | 10536 | TTTTGATGCCAAAACTATTAGAT |
| 10105 | TATCTAATAGTTTTGGCATCAAA | 10537 | TTTGATGCCAAAACTATTAGATA |
| 10106 | TTATCTAATAGTTTTGGCATCAA | 10538 | TTGATGCCAAAACTATTAGATAA |
| 10107 | TTTATCTAATAGTTTTGGCATCA | 10539 | TGATGCCAAAACTATTAGATAAA |
| 10108 | TTTTATCTAATAGTTTTGGCATC | 10540 | GATGCCAAAACTATTAGATAAAA |
| 10109 | CTTTTATCTAATAGTTTTGGCAT | 10541 | ATGCCAAAACTATTAGATAAAAG |
| 10110 | CCTTTTATCTAATAGTTTTGGCA | 10542 | TGCCAAAACTATTAGATAAAAGG |
| 10111 | ACCTTTTATCTAATAGTTTTGGC | 10543 | GCCAAAACTATTAGATAAAAGGT |
| 10112 | AACCTTTTATCTAATAGTTTTGG | 10544 | CCAAAACTATTAGATAAAAGGTT |
| 10113 | TAACCTTTTATCTAATAGTTTTG | 10545 | CAAAACTATTAGATAAAAGGTTA |
| 10114 | TTAACCTTTTATCTAATAGTTTT | 10546 | AAAACTATTAGATAAAAGGTTAA |
| 10115 | ATTAACCTTTTATCTAATAGTTT | 10547 | AAACTATTAGATAAAAGGTTAAT |
| 10116 | GATTAACCTTTTATCTAATAGTT | 10548 | AACTATTAGATAAAAGGTTAATC |
| 10117 | AGATTAACCTTTTATCTAATAGT | 10549 | ACTATTAGATAAAAGGTTAATCT |
| 10118 | TAGATTAACCTTTTATCTAATAG | 10550 | CTATTAGATAAAAGGTTAATCTA |
| 10119 | GTAGATTAACCTTTTATCTAATA | 10551 | TATTAGATAAAAGGTTAATCTAC |
| 10120 | TGTAGATTAACCTTTTATCTAAT | 10552 | ATTAGATAAAAGGTTAATCTACA |
| 10121 | ATGTAGATTAACCTTTTATCTAA | 10553 | TTAGATAAAAGGTTAATCTACAT |
| 10122 | GATGTAGATTAACCTTTTATCTA | 10554 | TAGATAAAAGGTTAATCTACATC |
| 10123 | GGATGTAGATTAACCTTTTATCT | 10555 | AGATAAAAGGTTAATCTACATCC |
| 10124 | GGGATGTAGATTAACCTTTTATC | 10556 | GATAAAAGGTTAATCTACATCCC |
| 10125 | AGGGATGTAGATTAACCTTTTAT | 10557 | ATAAAAGGTTAATCTACATCCCT |
| 10126 | TAGGGATGTAGATTAACCTTTTA | 10558 | TAAAAGGTTAATCTACATCCCTA |
| 10127 | GTAGGGATGTAGATTAACCTTTT | 10559 | AAAAGGTTAATCTACATCCCTAC |
| 10128 | AGTAGGGATGTAGATTAACCTTT | 10560 | AAAGGTTAATCTACATCCCTACT |
| 10129 | TAGTAGGGATGTAGATTAACCTT | 10561 | AAGGTTAATCTACATCCCTACTA |
| 10130 | CTAGTAGGGATGTAGATTAACCT | 10562 | AGGTTAATCTACATCCCTACTAG |
| 10131 | TCTAGTAGGGATGTAGATTAACC | 10563 | GGTTAATCTACATCCCTACTAGA |
| 10132 | TTCTAGTAGGGATGTAGATTAAC | 10564 | GTTAATCTACATCCCTACTAGAA |
| 10133 | ATTCTAGTAGGGATGTAGATTAA | 10565 | TTAATCTACATCCCTACTAGAAT |
| 10134 | AATTCTAGTAGGGATGTAGATTA | 10566 | TAATCTACATCCCTACTAGAATT |
| 10135 | GAATTCTAGTAGGGATGTAGATT | 10567 | AATCTACATCCCTACTAGAATTC |
| 10136 | AGAATTCTAGTAGGGATGTAGAT | 10568 | ATCTACATCCCTACTAGAATTCT |
| 10137 | GAGAATTCTAGTAGGGATGTAGA | 10569 | TCTACATCCCTACTAGAATTCTC |
| 10138 | TGAGAATTCTAGTAGGGATGTAG | 10570 | CTACATCCCTACTAGAATTCTCA |
| 10139 | ATGAGAATTCTAGTAGGGATGTA | 10571 | TACATCCCTACTAGAATTCTCAT |
| 10140 | TATGAGAATTCTAGTAGGGATGT | 10572 | ACATCCCTACTAGAATTCTCATA |
| 10141 | GTATGAGAATTCTAGTAGGGATG | 10573 | CATCCCTACTAGAATTCTCATAC |

*At least one nucleoside linkage of the nucleobase sequence is selected from a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

Antisense Oligonucleotides with One or More Locked Nucleic Acids (LNAs)

In various embodiments, antisense oligonucleotides disclosed herein (e.g., AON parent oligonucleotides and/or AON oligonucleotide variants) comprise one or more locked nucleic acids (LNAs). In particular embodiments, an antisense oligonucleotide includes one LNA. In particular embodiments, an antisense oligonucleotide includes two LNAs. In particular embodiments, an antisense oligonucleotide includes three LNAs. Generally, a LNA refers to nucleic acid monomers having a bridge (e.g., methylene, ethylene, aminooxy, or oxyimino bridge) connecting two carbon atoms between the 4′ and 2′ position of the nucleoside sugar unit, thereby forming a bicyclic sugar. Generally, a LNA includes a nucleobase (e.g., one of A, C, G, or T). In various embodiments, a LNA with a cytosine nucleobase includes a modified cytosine nucleobase (e.g., a 5-methylcytosine nucleobase).

In some embodiments, an antisense oligonucleotide disclosed herein includes, if counting from 5′ to 3′, a LNA at a 4^{th} position of the antisense oligonucleotide. In some embodiments, an antisense oligonucleotide disclosed herein includes, if counting from 5′ to 3′, a LNA at a 7^{th} position of the antisense oligonucleotide. In some embodiments, an antisense oligonucleotide disclosed herein includes, if counting from 5′ to 3′, a LNA at a 9^{th} position of the antisense oligonucleotide. In some embodiments, an antisense oligonucleotide disclosed herein includes, if counting from 5′ to 3′, a LNA at a 12^{th} position of the antisense oligonucleotide. In some embodiments, an antisense oligonucleotide disclosed herein includes, if counting from 5′ to 3′, a LNA at a 15^{th} position of the antisense oligonucleotide. In some embodiments, an antisense oligonucleotide disclosed herein includes, if counting from 5′ to 3′, a LNA at a 17^{th} position of the antisense oligonucleotide. In some embodiments, an antisense oligonucleotide disclosed herein includes, if counting from 5′ to 3′, a LNA at a 20^{th} position of the antisense oligonucleotide.

In various embodiments, antisense oligonucleotides disclosed herein (e.g., SMN2 parent oligonucleotides and/or SMN2 oligonucleotide variants) comprise two LNAs located at two different positions of the antisense oligonucleotide. In some embodiments, an antisense oligonucleotide disclosed herein includes, if counting from 5′ to 3′, a first LNA at a fourth position of the antisense oligonucleotide and a second LNA at a 20^{th} position of the antisense oligonucleotide. In some embodiments, an antisense oligonucleotide disclosed herein includes, if counting from 5′ to 3′, a first LNA at a 7^{th} position of the antisense oligonucleotide and a second LNA at a 15^{th} position of the antisense oligonucleotide. In some embodiments, an antisense oligonucleotide disclosed herein includes, if counting from 5′ to 3′, a first LNA at a 7^{th} position of the antisense oligonucleotide and a second LNA at a 17^{th} position of the antisense oligonucleotide. In some embodiments, an antisense oligonucleotide disclosed herein includes, if counting from 5′ to 3′, a first LNA at a 9^{th} position of the antisense oligonucleotide and a second LNA at a 17^{th} position of the antisense oligonucleotide.

In various embodiments, antisense oligonucleotides disclosed herein (e.g., SMN2 parent oligonucleotides and/or SMN2 oligonucleotide variants) comprise three LNAs located at three different positions of the antisense oligonucleotide. In some embodiments, an antisense oligonucleotide disclosed herein includes, if counting from 5′ to 3′, a first LNA at a 4^{th} position of the antisense oligonucleotide, a second LNA at a 12^{th} position of the antisense oligonucleotide, and a third LNA at a 20^{th} position of the antisense oligonucleotide.

Antisense Oligonucleotides with One or More Spacers

In various embodiments, antisense oligonucleotides comprise one or more spacers. In particular embodiments, an antisense oligonucleotide includes one spacer. In particular embodiments, an antisense oligonucleotide includes two spacers. In particular embodiments, an antisense oligonucleotide includes three spacers. Generally, a spacer refers to a nucleoside-replacement group lacking a nucleotide base and wherein the nucleoside sugar moiety is replaced by a non-sugar substitute group. The non-sugar substitute group is not capable of linking to a nucleobase, but is capable of linking with the 3′ and 5′ positions of nucleosides adjacent to the spacer through an internucleoside linking group.

In certain embodiments, an oligonucleotide with one or more spacers, such as disclosed herein, may be an oligonucleotide with 5 to 100 oligonucleotide units in length, for example, 10 to 60 oligonucleotide units in length, for example, 12 to 50 oligonucleotide units in length, 14 to 40 oligonucleotide units in length, 10 to 30 oligonucleotide units in length, for example, 14 to 30 oligonucleotide units in length, for example, 14 to 25 or 15 to 22 oligonucleotide units in length, or 18, 19, 20, 21, 22, 23, 24, or 25 oligonucleotide units in length. As used herein, an “oligonucleotide unit” refers to either a nucleoside (e.g., a nucleoside which includes a sugar and/or a nucleobase) or a nucleoside-replacement group (e.g., a spacer) of the oligonucleotide.

In particular embodiments, oligonucleotides with one or more spacers are 25 oligonucleotide units in length. In particular embodiments, the oligonucleotides with one or more spacers are 23 oligonucleotide units in length. In particular embodiments, the oligonucleotides with one or more spacers are 21 oligonucleotide units in length. In particular embodiments, the oligonucleotides with one or more spacers are 19 oligonucleotide units in length. In various embodiments, the oligonucleotides with one or more spacers are at least 18, at least 19, at least 20, at least 21, at least 22, at least 23, at least 24, or at least 25 oligonucleotide units in length. In various embodiments, the oligonucleotides with one or more spacers are at least 18 oligonucleotide units in length. In various embodiments, the oligonucleotides with one or more spacers are at least 19 oligonucleotide units in length. In various embodiments, the oligonucleotides with one or more spacers are at least 20 oligonucleotide units in length. In various embodiments, the oligonucleotides with one or more spacers are at least 21 oligonucleotide units in length. In various embodiments, the oligonucleotides with one or more spacers are at least 22 oligonucleotide units in length. In various embodiments, the oligonucleotides with one or more spacers are at least 23 oligonucleotide units in length. In various embodiments, the oligonucleotides with one or more spacers are at least 24 oligonucleotide units in length. In various embodiments, the oligonucleotides with one or more spacers are at least 25 oligonucleotide units in length.

In various embodiments, a STMN2 AON comprises a sequence that shares at least 80% identity with an equal length portion of any one of SEQ ID NOs: 1451-1664. In various embodiments, a STMN2 AON comprises a sequence that shares at least 85% identity with an equal length portion of any one of SEQ ID NOs: 1451-1664. In various embodiments, a STMN2 AON comprises a sequence that shares at least 90% identity with an equal length portion of any one of SEQ ID NOs: 1451-1664. In various embodiments, a STMN2 AON comprises a sequence that shares at least 95% identity with an equal length portion of any one of SEQ ID NOs: 1451-1664. In various embodiments, a STMN2 AON comprises a sequence that shares 100% identity with an equal length portion of any one of SEQ ID NOs: 1451-1664.

In various embodiments, a KCNQ2 AON comprises a sequence that shares at least 80% identity with an equal length portion of any one of SEQ ID NOs: 4402-4468, SEQ ID NOs: 4469-4476, and SEQ ID NOs: 4477-4530. In various embodiments, a KCNQ2 AON comprises a sequence that shares at least 85% identity with an equal length portion of any one of SEQ ID NOs: 4402-4468, SEQ ID NOs: 4469-4476, and SEQ ID NOs: 4477-4530. In various embodiments, a KCNQ2 AON comprises a sequence that shares at least 90% identity with an equal length portion of any one of SEQ ID NOs: 4402-4468, SEQ ID NOs: 4469-4476, and SEQ ID NOs: 4477-4530. In various embodiments, a KCNQ2 AON comprises a sequence that shares at least 95% identity with an equal length portion of any one of SEQ ID NOs: 4402-4468, SEQ ID NOs: 4469-4476, and SEQ ID NOs: 4477-4530. In various embodiments, a KCNQ2 AON comprises a sequence that shares 100% identity with an equal length portion of any one of SEQ ID NOs: 4402-4468, SEQ ID NOs: 4469-4476, and SEQ ID NOs: 4477-4530.

In various embodiments, a UNC13A AON comprises a sequence that shares at least 80% identity with an equal length portion of any one of SEQ ID NOs: 9596-9696 or SEQ ID NOs: 10670-10779. In various embodiments, a UNC13A AON comprises a sequence that shares at least 85% identity with an equal length portion of any one of SEQ ID NOs: 9596-9696 or SEQ ID NOs: 10670-10779. In various embodiments, a UNC13A AON comprises a sequence that shares at least 90% identity with an equal length portion of any one of SEQ ID NOs: 9596-9696 or SEQ ID NOs: 10670-10779. In various embodiments, a UNC13A AON comprises a sequence that shares at least 95% identity with an equal length portion of any one of SEQ ID NOs: 9596-9696 or SEQ ID NOs: 10670-10779. In various embodiments, a UNC13A AON comprises a sequence that shares 100% identity with an equal length portion of any one of SEQ ID NOs: 9596-9696 or SEQ ID NOs: 10670-10779.

In various embodiments, a SMN2 AON comprises a sequence that shares at least 80% identity with an equal length portion of any one of SEQ ID NOs: 10574-10640 and SEQ ID NOs: 10644-10651. In various embodiments, a SMN2 AON comprises a sequence that shares at least 85% identity with an equal length portion of any one of SEQ ID NOs: 10574-10640 and SEQ ID NOs: 10644-10651. In various embodiments, a SMN2 AON comprises a sequence that shares at least 90% identity with an equal length portion of any one of SEQ ID NOs: 10574-10640 and SEQ ID NOs: 10644-10651. In various embodiments, a SMN2 AON comprises a sequence that shares at least 95% identity with an equal length portion of any one of SEQ ID NOs: 10574-10640 and SEQ ID NOs: 10644-10651. In various embodiments, a SMN2 AON comprises a sequence that shares at least 99% identity with an equal length portion of any one of SEQ ID NOs: 10574-10640 and SEQ ID NOs: 10644-10651. In various embodiments, a SMN2 AON comprises a sequence that shares 100% identity with an equal length portion of any one of SEQ ID NOs: 10574-10640 and SEQ ID NOs: 10644-10651.

In some embodiments, the spacer is of Formula (X):

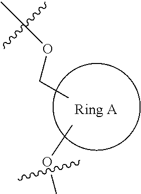

wherein ring A is as defined herein.

In some embodiments, the spacer is of Formula (Xa):

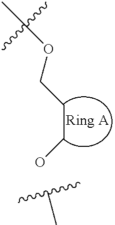

wherein ring A is as defined herein and the —CH₂—O— group is on a ring A atom adjacent to the —O— group.

As generally defined herein, ring A of formulae (X) and (Xa), is an optionally substituted 4-8 member monocyclic cycloalkyl group (e.g. ring A is cyclobutyl, cyclopentyl, cyclohexyl, cycloheptyl, cyclooctyl) or a 4-8 member monocyclic heterocyclyl group, wherein the heterocyclyl group contains 1 or 2 heteroatoms selected from O, S and N (e.g. ring A is oxetanyl, tetrahydrofuranyl, tetrahydropyranyl, 1,4-dioxanyl, pyrrolidinyl, piperidinyl, piperazinyl, morpholinyl, azepanyl). In some embodiments, ring A is tetrahydrofuranyl. In some embodiments, ring A is tetrahydropyranyl. In some embodiments, ring A is pyrrolidinyl. In some embodiments, ring A is cyclopentyl. In some embodiments, the monocyclic cycloalkyl or monocyclic heterocyclyl is not further substituted. In some embodiments, the cycloalkyl or heterocyclyl is further substituted with 0, 1, 2 or 3 substituents selected from halo (e.g., —F, —Cl), —OMe, —OEt —O(CH₂)OMe, —O(CH₂)₂OMe and CN.

In some embodiments, tetrahydrofuranyl is substituted with 1 or 2 substituents selected from halo (e.g., —F, —Cl), —OMe, —OEt —O(CH₂)OMe, —O(CH₂)₂OMe and CN. In some embodiments, tetrahydrofuranyl is substituted with 2 substituents selected from halo (e.g., —F, —Cl), —OMe, —OEt —O(CH₂)OMe, —O(CH₂)₂OMe and CN. In some embodiments, tetrahydrofuranyl is substituted with 1 substituent selected from halo (e.g., —F, —Cl), —OMe, —OEt —O(CH₂)OMe, —O(CH₂)₂OMe and CN. In some embodiments, tetrahydrofuranyl is substituted with —O(CH₂)₂OMe.

In some embodiments, the spacer is represented by Formula (I), wherein:

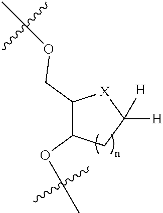

X is selected from —CH₂— and —O—; and
n is 0, 1, 2 or 3.

In some embodiments, the spacer is represented by Formula (I′), wherein:

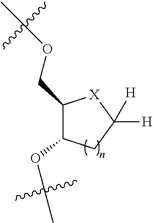

X is selected from —CH₂— and —O—; and
n is 0, 1, 2 or 3.

In some embodiments, the spacer is represented by Formula (Ia), wherein:

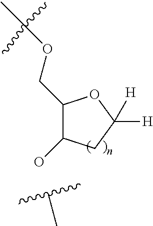

and n is 0, 1, 2 or 3.

In some embodiments, the spacer is represented by Formula (Ia′), wherein:

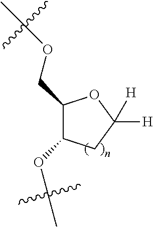

and n is 0, 1, 2 or 3.

As generally defined herein, X is selected from —CH₂— and —O—. In some embodiments, X is —CH₂—. In other embodiments, X is —O—.

As generally defined herein, n is 0, 1, 2 or 3. In some embodiments, n is 0. In some embodiments, n is 1 or 2. In some embodiments, n is 1. In other embodiments, n is 2. In certain embodiments, n is 3.

In some embodiments, the spacer is represented by Formula (II), wherein:

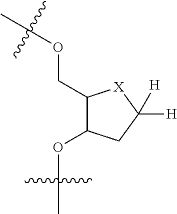

X is selected from —CH₂— and —O—.

In some embodiments, the spacer is represented by Formula (II′), wherein:

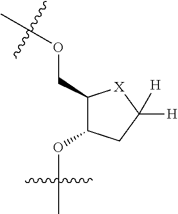

X is selected from —CH₂— and —O.

In some embodiments, the spacer is represented by Formula (Iia), wherein:

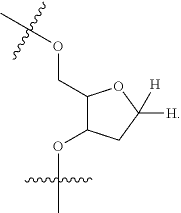

In some embodiments, the spacer is represented by Formula (Iia′), wherein:

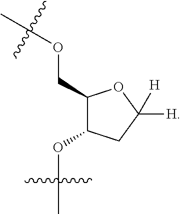

In some embodiments, the spacer is represented by Formula (IIib), wherein:

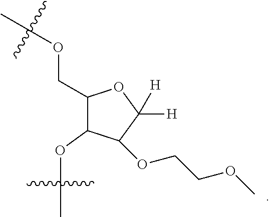 [SMILES: *OCC1OCC(OCCOC)C1O*]

In some embodiments, the spacer is represented by Formula (IIib′), wherein:

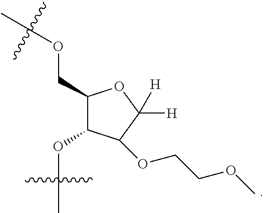 [SMILES: *OC[C@H]1OCC(OCCOC)[C@@H]1O*]

In some embodiments, the spacer is represented by Formula (III), wherein:

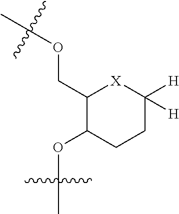

X is selected from —CH₂— and —O—.

In some embodiments, the spacer is represented by Formula (III′), wherein:

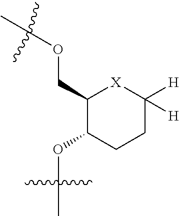

X is selected from —CH₂— and —O.

In some embodiments, the spacer is represented by Formula (IIIa), wherein:

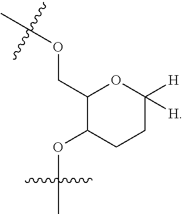

In some embodiments, the spacer is represented by Formula (IIIa′), wherein:

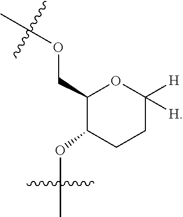

In some embodiments, the open positions of Formulae (I), (I′), (Ia), (Ia′), (II), (II′), (Iia), (Iia′), (III), (III′), (IIIa) and (IIIa′) (i.e., the positions not specifically depicted as bearing exclusively hydrogen atoms, including the —CH₂— group of X) are further substituted with 0-3 substituents selected from halo (e.g., —F, —Cl), —OMe, —OEt —O(CH₂)OMe, —O(CH₂)₂OMe and CN. In some embodiments, Formulae (I), (I′), (Ia), (Ia′), (II), (II′), (Iia), (Iia′), (III), (III′), (IIIa) and (IIIa′) are not further substituted.

### STMN2

As described further below, a STMN2 oligonucleotide with one or more spacers is described in reference to a corresponding STMN2 parent oligonucleotide. In various embodiments, a STMN2 oligonucleotide with a spacer differs from a STMN2 parent oligonucleotide in that the spacer replaces a nucleoside in the STMN2 parent oligonucleotide. As used hereafter, the “position” of the STMN2 oligonucleotide refers to a particular location as counted from the 5′ end of the STMN2 oligonucleotide. In various embodiments, the spacer replaces a nucleoside at any one of positions 1, 2, 3, 4, 5, 6, 7, 8, 9, 10, 11, 12, 13, 14, 15, 16, 17, 18, 19, 20, 21, 22, 23, 24, or 25 of the STMN2 parent oligonucleotide. In particular embodiments, a spacer replaces a nucleoside at one of positions 7, 8, 11, 14, 16, 19, or 22 of the STMN2 parent oligonucleotide.

In various embodiments, a STMN2 oligonucleotide includes one spacer that replaces a nucleoside in the STMN2 parent oligonucleotide (e.g., one spacer replaces one nucleoside of the STMN2 parent oligonucleotide). In particular embodiments, the spacer replaces a nucleoside between positions 9 and 15 of the STMN2 parent oligonucleotide. In particular embodiments, the spacer replaces a nucleoside between positions 9 and 12 of the STMN2 parent oligonucleotide. In particular embodiments, the spacer replaces a nucleoside at position 10 of the STMN2 parent oligonucleotide. In particular embodiments, the spacer replaces a nucleoside at position 11 of the STMN2 parent oligonucleotide. In particular embodiments, the spacer replaces a nucleoside at position 12 of the STMN2 parent oligonucleotide. In particular embodiments, the spacer replaces a nucleoside between positions 12 and 16 of the STMN2 parent oligonucleotide. In particular embodiments, the spacer replaces a nucleoside at position 15 of the STMN2 parent oligonucleotide.

In various embodiments, a STMN2 oligonucleotide including one spacer has 2 segments, where at least one of the 2 segments has at most 11 linked nucleosides. For example, the STMN2 oligonucleotide may be 23 oligonucleotide units in length, and the spacer can be located at position 12. Therefore, the STMN2 oligonucleotide has 2 segments divided by the spacer, where both of the 2 segments are 11 nucleobases in length. In various embodiments, a STMN2 oligonucleotide including one spacer has 2 segments, where at least one of the 2 segments has at most 10 linked nucleosides. For example, the STMN2 oligonucleotide may be 21 oligonucleotide units in length, and the spacer can be located at position 11. Therefore, the STMN2 oligonucleotide has 2 segments divided by the spacer, where both of the 2 segments are 10 nucleobases in length. As another example, the STMN2 oligonucleotide may be 25 oligonucleotide units in length, and the spacer can be located at position 15. Therefore, the STMN2 oligonucleotide has 2 segments divided by the spacer, where one of the 2 segments is 14 nucleobases in length and the second of the 2 segments is 10 nucleobases in length.

In various embodiments, a STMN2 oligonucleotide includes two spacers that each replace a nucleoside in the STMN2 parent oligonucleotide (e.g., two spacers replace two separate nucleosides of the STMN2 parent oligonucleotide). In various embodiments, a first spacer and a second spacer are separated by at least 5 nucleobases, at least 6 nucleobases, at least 7 nucleobases, at least 8 nucleobases, at least 9 nucleobases, or at least 10 nucleobases in the oligonucleotide. In particular embodiments, a first spacer and a second spacer are separated by at least 5 nucleobases, at least 6 nucleobases, or at least 7 nucleobases. In particular embodiments, the first spacer and the second spacer are not adjacent to one another in the oligonucleotide.

In particular embodiments, the first spacer replaces a nucleoside between positions 7 and 11 of the STMN2 parent oligonucleotide. In various embodiments, the first spacer replaces a nucleoside between positions 8 and 11, positions 9 and 11, positions 10 and 11, positions 7 and 10, positions 7 and 9, positions 7 and 8, positions 8 and 10, positions 8 and 9, or positions 9 and 10 of the STMN2 parent oligonucleotide. In particular embodiments, the second spacer replaces a nucleoside between positions 14 and 22 of the STMN2 parent oligonucleotide. In various embodiments, the second spacer replaces a nucleoside between positions 15 and 22, positions 16 and 22, positions 17 and 22, position 18 and 22, position 19 and 22, positions 20 and 22, positions 21 and 22, positions 15 and 21, position 16 and 21, positions 17 and 21, positions 18 and 21, positions 19 and 21, positions 20 and 21, positions 15 and 20, positions 16 and 20, positions 17 and 20, positions 18 and 20, positions 19 and 20, positions 15 and 19, positions 16 and 19, positions 17 and 19, positions 18 and 19, positions 15 and 18, position 16 and 18, position 17 and 18, positions 15 and 17, positions 16 and 17, or positions 15 and 16 of the STMN2 parent oligonucleotide.

In preferred embodiments, the first spacer replaces a nucleoside at position 7 of the STMN2 parent oligonucleotide and the second spacer replaces a nucleoside at position 14 of the STMN2 parent oligonucleotide. In preferred embodiments, the first spacer replaces a nucleoside at position 8 of the STMN2 parent oligonucleotide and the second spacer replaces a nucleoside at position 16 of the STMN2 parent oligonucleotide. In preferred embodiments, the first spacer replaces a nucleoside at position 11 of the STMN2 parent oligonucleotide and the second spacer replaces a nucleoside at position 22 of the STMN2 parent oligonucleotide. In preferred embodiments, the first spacer replaces a nucleoside at position 9 of the STMN2 parent oligonucleotide and the second spacer replaces a nucleoside at position 19 of the STMN2 parent oligonucleotide.

In various embodiments, a STMN2 oligonucleotide includes three spacers that each replace a nucleoside in the STMN2 parent oligonucleotide (e.g., three spacers replace three separate nucleosides of the STMN2 parent oligonucleotide). In particular embodiments, the first spacer replaces a nucleoside between positions 7 and 11 of the STMN2 parent oligonucleotide. In particular embodiments, the second spacer replaces a nucleoside between positions 14 and 22 of the STMN2 parent oligonucleotide. In particular embodiments, the third spacer replaces a nucleoside between positions 21 and 24 of the STMN2 parent oligonucleotide. In some embodiments, the first spacer replaces a nucleoside between positions 2 and 5 of the STMN2 parent oligonucleotide. In particular embodiments, the second spacer replaces a nucleoside between positions 8 and 12 of the STMN2 parent oligonucleotide. In particular embodiments, the third spacer replaces a nucleoside between positions 18 and 22 of the STMN2 parent oligonucleotide.

In various embodiments, the three spacers in a STMN2 oligonucleotide are positioned such that each of the four segments of the STMN2 oligonucleotide are at most 7 linked nucleosides in length. For example, a STMN2 oligonucleotide may have a first segment with 7 linked nucleosides connected to a first spacer, then a second segment with 7 linked nucleosides connected on one end to the first spacer and connected on another end to a second spacer, then a third segment with 6 linked nucleosides connected on one end to the second spacer and connected on another end to a third spacer, then a fourth segment with 6 linked nucleosides connected to the third spacer.

In various embodiments, the one or more spacers are positioned in the oligonucleotide to replace one or more adenosine or thymine nucleosides (as opposed to guanine or cytosine nucleosides). For example, the one or more spacers can replace one, two, three, four, five, six, seven, eight, or nine adenosine or thymine nucleosides in the oligonucleotide. In various embodiments, the one or more spacers are positioned in the oligonucleotide to replace one or more guanine or cytosine nucleosides (as opposed to adenosine or thymine nucleosides). For example, the one or more spacers can replace one, two, three, four, five, six, seven, eight, or nine guanine or cytosine nucleosides in the oligonucleotide. In various embodiments, the spacers are positioned in the oligonucleotide to replace an equal number of adenosine/thymine nucleosides and guanine/cytosine nucleosides. For example, a first spacer in the oligonucleotide may replace an adenosine/thymine nucleoside and a second spacer in the oligonucleotide may replace a guanine/cytosine nucleoside.

In various embodiments, the one or more spacers are positioned in the oligonucleotide to control the sequence content in the oligonucleotide. For example, the one or more spacers are positioned such that at least one of the spacers is located adjacent to a guanine group. In various embodiments, an oligonucleotide with spacers can include one spacer adjacent to a guanine group, two spacers adjacent to guanine groups, three spacers adjacent to guanine groups, four spacers adjacent to guanine groups, or five spacers adjacent to guanine groups. In one embodiment, if counting from the 5′ end of the oligonucleotide, a spacer immediately precedes a guanine group in the sequence. Thus, in various embodiments, an oligonucleotide with spacers can include one spacer that immediately precedes a guanine group, two spacers that each immediately precede a guanine group, three spacers that each immediately precede a guanine group, four spacers that each immediately precede a guanine group, or five spacers that each immediately precede a guanine group. In one embodiment, if counting from the 5′ end of the oligonucleotide, a guanine group is immediately succeeded by a spacer. Thus, in various embodiments, an oligonucleotide with spacers can include one spacer that immediately succeeds a guanine group, two spacers that each immediately succeed a guanine group, three spacers that each immediately succeed a guanine group, four spacers that each immediately succeed a guanine group, or five spacers that each immediately succeed a guanine group. In various embodiments, the spacers in the oligonucleotide can be positioned to maximize the number of spacers adjacent to guanine groups.

In various embodiments, the one or more spacers are positioned in the oligonucleotide to replace one or more adenosine or thymine nucleosides such that the one or more spacers are located adjacent guanine groups. For example, two spacers can replace adenosine or thymine nucleosides in the oligonucleotide, each of the two spacers being located adjacent to a guanine group.

In various embodiments, the STMN2 oligonucleotide with one or more spacers has a particular GC content. As used herein, GC content (or guanine-cytosine content) is the percentage of nitrogenous bases in the oligonucleotide that are either guanine (G) or cytosine (C). In various embodiments, the STMN2 oligonucleotide with one or more spacers has at least 10% GC content, at least 20% GC content, at least 25% GC content, at least 30% GC content, at least 35% GC content, at least 40% GC content, at least 45% GC content, at least 50% GC content, at least 55% GC content, at least 60% GC content, at least 65% GC content, at least 75% GC content, at least 80% GC content, at least 85% GC content, at least 90% GC content, or at least 95% GC content. In particular embodiments, the STMN2 oligonucleotide with one or more spacers has at least 30% GC content. In particular embodiments, the STMN2 oligonucleotide with one or more spacers has at least 40% GC content. In various embodiments, the one or more spacers are positioned in the STMN2 oligonucleotide to maximize GC content. For example, instead of selecting a guanine or cytosine for replacement by a spacer in the STMN2 oligonucleotide, a thymine or adenine can be selected for replacement by a spacer.

In various embodiments, a STMN2 oligonucleotide with spacers is designed such that 1) each segment of the STMN2 oligonucleotide has at most 7 linked nucleosides and 2) at least two, three, or four spacers are positioned adjacent to a guanine group. In some embodiments, a STMN2 oligonucleotide with spacers is designed such that 1) each segment of the STMN2 oligonucleotide has at most 7 linked nucleosides and 2) each of two spacers precede a guanine group.

In various embodiments, the inclusion of one or more spacers in the STMN2 oligonucleotide does not decrease the effectiveness of the STMN2 oligonucleotide with the spacers in restoring full length STMN2 protein or full length STMN2 mRNA in comparison to the effect of a corresponding STMN2 parent oligonucleotide. In various embodiments, the inclusion of one or more spacers in the STMN2 oligonucleotide increases the effectiveness of the STMN2 oligonucleotide with the spacers in restoring full length STMN2 protein or full length STMN2 mRNA in comparison to the effect of a corresponding STMN2 parent oligonucleotide. In various embodiments, the inclusion of one or more spacers in the STMN2 oligonucleotide does not decrease the effectiveness of the STMN2 oligonucleotide with the spacers in reducing quantity of STMN2 transcripts with a cryptic exon in comparison to the effect of a corresponding STMN2 parent oligonucleotide. In various embodiments, the inclusion of one or more spacers in the STMN2 oligonucleotide increases the effectiveness of the STMN2 oligonucleotide with the spacers in reducing quantity of STMN2 transcripts with a cryptic exon in comparison to the effect of a corresponding STMN2 parent oligonucleotide.

Tables 12A, 12B, 13, and 14 document example STMN2 oligonucleotides with one or more spacers and their relation to corresponding STMN2 parent oligonucleotides. Each STMN2 oligonucleotide is assigned a sequence name. As used hereafter, the nomenclature of the sequence name is expressed as “X_spA” (for a STMN2 AON with one spacer), “X_spA_spB” (for a STMN2 AON with two spacers), or “X_spA_spB_spC” (for a STMN2 AON with three spacers). Here, “X” refers to the length of the STMN2 AON, “A” refers to the position in the STMN2 AON where the first spacer is located, “B” refers to the position in the STMN2 AON where the second spacer is located, and if present, “C” refers to the position in the STMN2 AON where the third spacer is located.

In various embodiments, STMN2 oligonucleotides include one spacer. In various embodiments, the STMN2 oligonucleotides are oligonucleotide variants, such as any one of a 23mer, 21mer, or 19mer. In various embodiments, the inclusion of a spacer divides up the STMN2 oligonucleotide into two separate segments, where at least one of the segments is at most 11 linked nucleosides in length. In various embodiments, the inclusion of a spacer divides up the STMN2 oligonucleotide into two separate segments, where at least one of the segments is at most 10 linked nucleosides in length.

In various embodiments, the spacer is located between positions 10 and 15 of the oligonucleotide. In various embodiments, the spacer is located between positions 10 and 12 of the oligonucleotide. In particular embodiments, the spacer is located at position 10 of the oligonucleotide. In particular embodiments, the spacer is located at position 11 of the oligonucleotide. In particular embodiments, the spacer is located at position 12 of the oligonucleotide. In particular embodiments, the spacer is located at position 15 of the oligonucleotide.

Example STMN2 AONs with one spacer are documented below in Table 12A.

TABLE 12A
Identification of STMN2 AONs with one spacer. Here, each STMN2 AON has 2
segments, where at least one of the segments has at most 11 linked nucleosides.

| | | Sequence | Sequence* (where X indicates a |
|---|---|---|---|
| | Relation to | ID | nucleoside of the STMN2 parent |
| | STMN2 | Number | oligonucleotide and S_{y} indicates |
| | oligonucleotide | (SEQ ID | presence of a Spacer where y |
| Sequence name | variant | NO) | denotes the position) (5′→3′) |
| STMN2 parent | N/A | 1522 | XXXXXXXXXXXXXXXXXXXXXXXXX |
| oligonucleotide | | | (25 mer) |
| STMN2 Oligonucleotide | Nucleoside at | 1523 | XXXXXXXXXXXXXXS₁₅XXXXXXXXXX |
| (25 mer) with Spacer at | position 15 of | | |
| position 15 | 25 mer is | | |
| (STMN2 AON 25_sp15) | substituted with | | |
| | a spacer | | |
| STMN2 oligonucleotide | N/A | 1524 | XXXXXXXXXXXXXXXXXXXXXXX |
| variant (23 mer) | | | (23 mer) |
| STMN2 Oligonucleotide | Nucleoside at | 1525 | XXXXXXXXXXXS₁₂XXXXXXXXXXX |
| (23 mer) with Spacer at | position 12 of | | |
| position 12 | 23 mer is | | |
| (STMN2 AON 23_sp12) | substituted with | | |
| | a spacer | | |
| STMN2 oligonucleotide | N/A | 1526 | XXXXXXXXXXXXXXXXXXXXX |
| variant (21 mer) | | | (21 mer) |
| STMN2 Oligonucleotide | Nucleoside at | 1527 | XXXXXXXXXXS₁₁XXXXXXXXXX |
| (21 mer) with Spacer at | position 11 of | | |
| position 11 | 21 mer is | | |
| (STMN2 AON 21_sp11) | substituted with | | |
| | a spacer | | |
| STMN2 oligonucleotide | N/A | 1528 | XXXXXXXXXXXXXXXXXXX |
| variant (19 mer) | | | (19 mer) |
| STMN2 Oligonucleotide | Nucleoside at | 1529 | XXXXXXXXXS₁₀XXXXXXXXX |
| (19 mer) with Spacer at | position 10 of | | |
| position 10 | 19 mer is | | |
| (STMN2 AON 19_sp10) | substituted with | | |
| | a spacer | | |

*At least one nucleoside linkage of the nucleobase sequence is selected from a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

In various embodiments, STMN2 oligonucleotides include two spacers. In various embodiments, the inclusion of a spacer divides up the STMN2 oligonucleotide into three separate segments, where at least one of the segments is at most 7 linked nucleosides in length. Example STMN2 AONs with two spacers are documented below in Table 12B.

TABLE 12B
Identification of STMN2 AONs with two spacers. Here, each STMN2 AON has 3
segments, where at least one of the segments has at most 7 linked nucleosides.

| | | | Sequence* (where X indicates a |
|---|---|---|---|
| | | | nucleoside of the STMN2 parent |
| | Relation to | Sequence ID | oligonucleotide and S_{y} indicates |
| | STMN2 parent | Number | presence of a Spacer where y |
| Sequence name | oligonucleotide | (SEQ ID NO) | denotes the position) (5′→3′) |
| STMN2 parent | N/A | 1530 | XXXXXXXXXXXXXXXXXXXXXXXXX |
| oligonucleotide | | | |
| STMN2 | Nucleosides at | 1531 | XXXXXXXXXXS₁₁XXXXXXXXXXS₂₂XXX |
| Oligonucleotide | positions 11 and | | |
| with Spacers at | 22 are each | | |
| positions 11 and 22 | substituted with | | |
| (STMN2 AON | a spacer | | |
| 25_sp11sp22) | | | |
| STMN2 | Nucleosides at | 1532 | XXXXXXS₇XXXXXXS₁₄XXXXXXXXXXX |
| Oligonucleotide | positions 7 and | | |
| with Spacers at | 14 are each | | |
| positions 7 and 14 | substituted with | | |
| (STMN2 AON | a spacer | | |
| 25_sp7sp14) | | | |
| STMN2 | Nucleosides at | 1533 | XXXXXXXS₈XXXXXXXXXXS₁₉XXXXXX |
| Oligonucleotide | positions 8 and | | |
| with Spacers at | 19 are | | |
| positions 8 and 19 | substituted with | | |
| (STMN2 AON | spacers | | |
| 25_sp8sp19) | | | |
| STMN2 | Nucleosides at | 1534 | XXXXXXXS₈XXXXXS₁₄XXXXXXXXXXX |
| Oligonucleotide | positions 8 and | | |
| with Spacers at | 14 are | | |
| positions 8 and 14 | substituted with | | |
| (STMN2 AON | spacers | | |
| 25_sp8sp14) | | | |
| STMN2 | Nucleosides at | 1535 | XXXXXXXXS₉XXXXS₁₄XXXXXXXXXXX |
| Oligonucleotide | positions 9 and | | |
| with Spacers at | 14 are | | |
| positions 9 and 14 | substituted with | | |
| (STMN2 AON | spacers | | |
| 25_sp9sp14) | | | |
| STMN2 | Nucleosides at | 1536 | XXXXXXXXXS₁₀XXXS₁₄XXXXXXXXXXX |
| Oligonucleotide | positions 10 and | | |
| with Spacers at | 14 are | | |
| positions 10 and 14 | substituted with | | |
| (STMN2 AON | spacers | | |
| 25_sp10sp14) | | | |
| STMN2 | Nucleosides at | 1537 | XXXXXXXXXXS₁₁XXS₁₄XXXXXXXXXXX |
| Oligonucleotide | positions 11 and | | |
| with Spacers at | 14 are | | |
| positions 11 and 14 | substituted with | | |
| (STMN2 AON | spacers | | |
| 25_sp11sp14) | | | |
| STMN2 | Nucleosides at | 1538 | XXXXXXXS₈XXXXXXS₁₅XXXXXXXXXX |
| Oligonucleotide | positions 8 and | | |
| with Spacers at | 15 are | | |
| positions 8 and 15 | substituted with | | |
| (STMN2 AON | spacers | | |
| 25_sp8sp15) | | | |
| STMN2 | Nucleosides at | 1539 | XXXXXXXXS₉XXXXXS₁₅XXXXXXXXXX |
| Oligonucleotide | positions 9 and | | |
| with Spacers at | 15 are | | |
| positions 9 and 15 | substituted with | | |
| (STMN2 AON | spacers | | |
| 25_sp9sp15) | | | |
| STMN2 | Nucleosides at | 1540 | XXXXXXXXXS₁₀XXXXS₁₅XXXXXXXXXX |
| Oligonucleotide | positions 10 and | | |
| with Spacers at | 15 are | | |
| positions 10 and 15 | substituted with | | |
| (STMN2 AON | spacers | | |
| 25_sp10sp15) | | | |
| STMN2 | Nucleosides at | 1541 | XXXXXXXXXXS₁₁XXXS₁₅XXXXXXXXXX |
| Oligonucleotide | positions 11 and | | |
| with Spacers at | 15 are | | |
| positions 11 and 15 | substituted with | | |
| (STMN2 AON | spacers | | |
| 25_sp11sp15) | | | |
| STMN2 | Nucleosides at | 1542 | XXXXXXXS₈XXXXXXXS₁₆XXXXXXXXX |
| Oligonucleotide | positions 8 and | | |
| with Spacers at | 16 are | | |
| positions 8 and 16 | substituted with | | |
| (STMN2 AON | spacers | | |
| 25_sp8sp16) | | | |
| STMN2 | Nucleosides at | 1543 | XXXXXXXXS₉XXXXXXS₁₆XXXXXXXXX |
| Oligonucleotide | positions 9 and | | |
| with Spacers at | 16 are | | |
| positions 9 and 16 | substituted with | | |
| (STMN2 AON | spacers | | |
| 25_sp9sp16) | | | |
| STMN2 | Nucleosides at | 1544 | XXXXXXXXXS₁₀XXXXXS₁₆XXXXXXXXX |
| Oligonucleotide | positions 10 and | | |
| with Spacers at | 16 are | | |
| positions 10 and 16 | substituted with | | |
| (STMN2 AON | spacers | | |
| 25_sp10sp16) | | | |
| STMN2 | Nucleosides at | 1545 | XXXXXXXXXXS₁₁XXXXS₁₆XXXXXXXXX |
| Oligonucleotide | positions 11 and | | |
| with Spacers at | 16 are | | |
| positions 11 and 16 | substituted with | | |
| (STMN2 AON | spacers | | |
| 25_sp11sp16) | | | |
| STMN2 | Nucleosides at | 1546 | XXXXXXXS₈XXXXXXXXS₁₇XXXXXXXX |
| Oligonucleotide | positions 8 and | | |
| with Spacers at | 17 are | | |
| positions 8 and 17 | substituted with | | |
| (STMN2 AON | spacers | | |
| 25_sp8sp17) | | | |
| STMN2 | Nucleosides at | 1547 | XXXXXXXXS₉XXXXXXXS₁₇XXXXXXXX |
| Oligonucleotide | positions 9 and | | |
| with Spacers at | 17 are | | |
| positions 9 and 17 | substituted with | | |
| (STMN2 AON | spacers | | |
| 25_sp9sp17) | | | |
| STMN2 | Nucleosides at | 1548 | XXXXXXXXXS₁₀XXXXXXS₁₇XXXXXXXX |
| Oligonucleotide | positions 10 and | | |
| with Spacers at | 17 are | | |
| positions 10 and 17 | substituted with | | |
| (STMN2 AON | spacers | | |
| 25_sp10sp17) | | | |
| STMN2 | Nucleosides at | 1549 | XXXXXXXXXXS₁₁XXXXXS₁₇XXXXXXXX |
| Oligonucleotide | positions 11 and | | |
| with Spacers at | 17 are | | |
| positions 11 and 17 | substituted with | | |
| (STMN2 AON | spacers | | |
| 25_sp11sp17) | | | |
| STMN2 | Nucleosides at | 1550 | XXXXXXXS₈XXXXXXXXXS₁₈XXXXXXX |
| Oligonucleotide | positions 8 and | | |
| with Spacers at | 18 are | | |
| positions 8 and 18 | substituted with | | |
| (STMN2 ATON | spacers | | |
| 25_sp8sp18) | | | |
| STMN2 | Nucleosides at | 1551 | XXXXXXXXS₉XXXXXXXXS₁₈XXXXXXX |
| Oligonucleotide | positions 9 and | | |
| with Spacers at | 18 are | | |
| positions 9 and 18 | substituted with | | |
| (STMN2 AON | spacers | | |
| 25_sp9sp18) | | | |
| STMN2 | Nucleosides at | 1552 | XXXXXXXXXS₁₀XXXXXXXS₁₈XXXXXXX |
| Oligonucleotide | positions 10 and | | |
| with Spacers at | 18 are | | |
| positions 10 and 18 | substituted with | | |
| (STMN2 AON | spacers | | |
| 25_sp10sp18) | | | |
| STMN2 | Nucleosides at | 1553 | XXXXXXXXXXS₁₁XXXXXXS₁₈XXXXXXX |
| Oligonucleotide | positions 11 and | | |
| with Spacers at | 18 are | | |
| positions 11 and 18 | substituted with | | |
| (STMN2 AON | spacers | | |
| 25_sp11sp18) | | | |
| STMN2 | Nucleosides at | 1554 | XXXXXXXXS₉XXXXXXXXXS₁₉XXXXXX |
| Oligonucleotide | positions 9 and | | |
| with Spacers at | 19 are | | |
| positions 9 and 19 | substituted with | | |
| (STMN2 AON | spacers | | |
| 25_sp9sp19) | | | |
| STMN2 | Nucleosides at | 1555 | XXXXXXXXXS₁₀XXXXXXXXS₁₉XXXXXX |
| Oligonucleotide | positions 10 and | | |
| with Spacers at | 19 are | | |
| positions 10 and 19 | substituted with | | |
| (STMN2 AON | spacers | | |
| 25_sp10sp19) | | | |
| STMN2 | Nucleosides at | 1556 | XXXXXXXXXXS₁₁XXXXXXXS₁₉XXXXXX |
| Oligonucleotide | positions 11 and | | |
| with Spacers at | 19 are | | |
| positions 11 and 19 | substituted with | | |
| (STMN2 AON | spacers | | |
| 25_sp11sp19) | | | |
| STMN2 | Nucleosides at | 1557 | XXXXXXXXS₉XXXXXXXXXXS₂₀XXXXX |
| Oligonucleotide | positions 9 and | | |
| with Spacers at | 20 are | | |
| positions 9 and 20 | substituted with | | |
| (STMN2 AON | spacers | | |
| 25_sp9sp20) | | | |
| STMN2 | Nucleosides at | 1558 | XXXXXXXXXS₁₀XXXXXXXXXS₂₀XXXXX |
| Oligonucleotide | positions 10 and | | |
| with Spacers at | 20 are | | |
| positions 10 and 20 | substituted with | | |
| (STMN2 AON | spacers | | |
| 25_sp10sp20) | | | |
| STMN2 | Nucleosides at | 1559 | XXXXXXXXXXS₁₁XXXXXXXXS₂₀XXXXX |
| Oligonucleotide | positions 11 and | | |
| with Spacers at | 20 are | | |
| positions 11 and 20 | substituted with | | |
| (STMN2 AON | spacers | | |
| 25_sp11sp20) | | | |
| STMN2 | Nucleosides at | 1560 | XXXXXXXXXS₁₀XXXXXXXXXXS₂₁XXXX |
| Oligonucleotide | positions 10 and | | |
| with Spacers at | 21 are | | |
| positions 10 and 21 | substituted with | | |
| (STMN2 AON | spacers | | |
| 25_sp10sp21) | | | |
| STMN2 | Nucleosides at | 1561 | XXXXXXXXXXS₁₁XXXXXXXXXS₂₁XXXX |
| Oligonucleotide | positions 11 and | | |
| with Spacers at | 21 are | | |
| positions 11 and 21 | substituted with | | |
| (STMN2 AON | spacers | | |
| 25_sp11sp21) | | | |
| STMN2 | Nucleosides at | 1562 | XXXS₄XXXXXXXXXXS₁₅XXXXXXXXXX |
| Oligonucleotide | positions 4 and | | |
| with Spacers at | 15 are | | |
| positions 4 and 15 | substituted with | | |
| (STMN2 AON | spacers | | |
| 25_sp4sp15) | | | |
| STMN2 | Nucleosides at | 1563 | XXXXXXS₇XXXXXXXXXXXS₁₉XXXXXX |
| Oligonucleotide | positions 7 and | | |
| with Spacers at | 19 are | | |
| positions 7 and 19 | substituted with | | |
| (STMN2 AON | spacers | | |
| 25_sp7sp19) | | | |
| STMN2 | Nucleosides at | 1564 | XXXXXXS₇XXXXXXXXXXS₁₈XXXXXXX |
| Oligonucleotide | positions 7 and | | |
| with Spacers at | 18 are | | |
| positions 7 and 18 | substituted with | | |
| (STMN2 AON | spacers | | |
| 25_sp7sp18) | | | |
| STMN2 | Nucleosides at | 1565 | XXXXXXXXS₉XXXXXXXXXXXS₂₁XXXX |
| Oligonucleotide | positions 9 and | | |
| with Spacers at | 21 are | | |
| positions 9 and 21 | substituted with | | |
| (STMN2 AON | spacers | | |
| 25_sp9sp21) | | | |

*At least one nucleoside linkage of the nucleobase sequence is selected from a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

In various embodiments, STMN2 oligonucleotides include three spacers. The inclusion of three spacers divides up the STMN2 oligonucleotide into four separate segments. In various embodiments, the three spacers are located at different positions of the STMN2 oligonucleotide such that each of the segments of the STMN2 oligonucleotide are at most 7 linked nucleosides in length.

In various embodiments, STMN2 AONs with three spacers are 27 oligonucleotide units in length. For example, a STMN2 AON with three spacers can divide up a STMN2 AON into four separate segments. In various embodiments, each of the four separate segments are 6 oligonucleotide units in length. For example, the STMN2 AON may be as follows (from 5′ to 3′ end): a first segment of 6 linked nucleosides, a first spacer, a second segment of 6 linked nucleosides, a second spacer, a third segment of 6 linked nucleosides, a third spacer, and a fourth segment of 6 linked nucleosides.

Example STMN2 AONs with three spacers are documented below in Table 13.

TABLE 13
Identification of STMN2 AONs or AON variants with three spacers. Here, each
STMN2 AON has 4 segments, where each segment has at most 7 linked nucleosides.

| | | | Sequence* (where X indicates a |
|---|---|---|---|
| | Relation to | | nucleoside of the STMN2 parent |
| | STMN2 | Sequence ID | oligonucleotide and S_{y} indicates |
| | parent | Number | presence of a Spacer where y denotes |
| Sequence name | oligonucleotide | (SEQ ID NO) | the position) (5′→3′) |
| STMN2 | Nucleosides | 1566 | XXXXXXXS₈XXXXXXXS₁₆XXXXXXXS₂₄X |
| Oligonucleotide | at positions 8 | | |
| with Spacers at | and 16 and 24 | | |
| positions 8 and 16 | are substituted | | |
| and 24 (STMN2 | with spacers | | |
| AON | | | |
| 25_sp8sp16sp24) | | | |
| STMN2 | Nucleosides | 1567 | XXXXXXXS₈XXXXXXXS₁₆XXXXXXS₂₃XX |
| Oligonucleotide | at positions 8 | | |
| with Spacers at | and 16 and 23 | | |
| positions 8 and 16 | are substituted | | |
| and 23 (STMN2 | with spacers | | |
| AON | | | |
| 25_sp8sp16sp23) | | | |
| STMN2 | Nucleosides | 1568 | XS₂XXXXXXXS₁₀XXXXXXXS₁₈XXXXXXX |
| Oligonucleotide | at positions 2 | | |
| with Spacers at | and 10 and 18 | | |
| positions 2 and 10 | are substituted | | |
| and 18 (STMN2 | with spacers | | |
| AON | | | |
| 25_sp8sp16sp23) | | | |
| STMN2 | Nucleosides | 1569 | XXS₃XXXXXXS₁₀XXXXXXXS₁₈XXXXXXX |
| Oligonucleotide | at positions 3 | | |
| with Spacers at | and 10 and 18 | | |
| positions 3 and 10 | are substituted | | |
| and 18 (STMN2 | with spacers | | |
| AON | | | |
| 25_sp8sp16sp23) | | | |
| STMN2 | Nucleosides | 1570 | XXXS₄XXXXXXXS₁₂XXXXXXS₁₉XXXXXX |
| Oligonucleotide | at positions 4 | | |
| with Spacers at | and 12 and 19 | | |
| positions 4 and 12 | are substituted | | |
| and 19 (STMN2 | with spacers | | |
| AON | | | |
| 25_sp4sp12sp19) | | | |
| STMN2 | Nucleosides | 1571 | XXXXXXXS₈XXXXS₁₃XXXXS₁₈XXXXXXX |
| Oligonucleotide | at positions 8 | | |
| with Spacers at | and 13 and 18 | | |
| positions 8 and 13 | are substituted | | |
| and 18 (STMN2 | with spacers | | |
| AON | | | |
| 25_sp8sp13sp18) | | | |
| STMN2 | Nucleosides | 1572 | XXXXS₅XXXXXXXS₁₃XXXXXXXS₂₁XXXX |
| Oligonucleotide | at positions 5 | | |
| with Spacers at | and 13 and 21 | | |
| positions 5 and 13 | are substituted | | |
| and 21 (STMN2 | with spacers | | |
| AON | | | |
| 25_sp5sp13sp21) | | | |
| STMN2 | Nucleosides | 1573 | XXXXXXS₇XXXXXS₁₃XXXXXS₁₉XXXXXX |
| Oligonucleotide | at positions 7 | | |
| with Spacers at | and 13 and 19 | | |
| positions 7 and 13 | are substituted | | |
| and 19 (STMN2 | with spacers | | |
| AON | | | |
| 25_sp7sp13sp19) | | | |
| STMN2 | Nucleosides | 1574 | XXXXXS₆XXXXXXS₁₃XXXXXXS₂₀XXXXX |
| Oligonucleotide | at positions 6 | | |
| with Spacers at | and 13 and 20 | | |
| positions 6 and 13 | are substituted | | |
| and 20 (STMN2 | with spacers | | |
| AON | | | |
| 25_sp6sp13sp20) | | | |
| STMN2 | Nucleosides | 1575 | XXXXXXXS₈XXS₁₁XXXXXXXS₁₉XXXXXX |
| Oligonucleotide | at positions 8 | | |
| with Spacers at | and 11 and 19 | | |
| positions 8 and 11 | are substituted | | |
| and 19 (STMN2 | with spacers | | |
| AON | | | |
| 25_sp8sp11sp19) | | | |
| STMN2 | Nucleosides | 1576 | XXXXXXXS₈XXS₁₁XXXXS₁₆XXXXXXX |
| Oligonucleotide | at positions 8 | | |
| with Spacers at | and 11 and 16 | | |
| positions 8 and 11 | are substituted | | |
| and 16 (STMN2 | with spacers | | |
| AON | | | |
| 23_sp8sp11sp16) | | | |
| STMN2 | Nucleosides | 1577 | XXXXXXS₇XXXXXXS₁₄XXXXXXXS₂₂XXX |
| Oligonucleotide | at positions 7 | | |
| with Spacers at | and 14 and 22 | | |
| positions 7 and 14 | are substituted | | |
| and 22 (STMN2 | with spacers | | |
| AON | | | |
| 23_sp7sp14sp22) | | | |

*At least one nucleoside linkage of the nucleobase sequence is selected from a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

In various embodiments, STMN2 AONs with one or more spacers are reduced in length in comparison to the STMN2 AONs described above in Tables 12B and 13. For example, such STMN2 AONs may be STMN2 oligonucleotide variants with one or more spacers. In various embodiments, the STMN2 oligonucleotide variants with one or more spacers are 23mers, 21mers, or 19mers. In various embodiments, STMN2 oligonucleotide variants include two spacers such that the STMN2 oligonucleotide variant includes three segments that are divided up by the two spacers. In various embodiments, at least one of the three segments has at most 7 linked nucleosides. In various embodiments, each of the three segments has at most 7 linked nucleosides. Example STMN2 oligonucleotide variants with one or more spacers are shown below in Table 14.

TABLE 14
STMN2 AONs or AON variants with three spacers. Here, each STMN2 AON variant
has 3 segments, where each segment has at most 7 linked nucleosides.

| | | | Sequence* (where X indicates a |
|---|---|---|---|
| | | | nucleoside of the STMN2 |
| | Relation to | | oligonucleotide variant and S_{y} |
| | STMN2 | Sequence ID | indicates presence of a |
| | oligonucleotide | Number | Spacer where y denotes the |
| Sequence name | variant | (SEQ ID NO) | position) (5′→3′) |
| STMN2 | N/A | 1578 | XXXXXXXXXXXXXXXXXXXXXXX |
| oligonucleotide | | | (23 mer) |
| variant (23 mer) | | | |
| STMN2 Variant | Nucleosides at | 1579 | XXXXXXXS₈XXXXXXXS₁₆XXXXXXX |
| Oligonucleotide | positions 8 and | | |
| (23 mer) with | 16 are | | |
| Spacers at | substituted with | | |
| positions 8 and | spacers | | |
| 16 (STMN2 | | | |
| AON variant | | | |
| 23_sp8sp16) | | | |
| STMN2 | N/A | 1580 | XXXXXXXXXXXXXXXXXXXXX |
| oligonucleotide | | | (21 mer) |
| variant (21 mer) | | | |
| STMN2 Variant | Nucleosides at | 1581 | XXXXS₅XXXXXXS₁₂XXXXXXXXX |
| Oligonucleotide | positions 5 and | | |
| (21 mer) with | 12 are | | |
| Spacers at | substituted with | | |
| positions 5 and | spacers | | |
| 12 (STMN2 | | | |
| AON variant | | | |
| 21_sp5sp12) | | | |
| STMN2 Variant | Nucleosides at | 1582 | XXXXXXXS₈XXXXXXXS₁₆XXXXX |
| Oligonucleotide | positions 8 and | | |
| (21 mer) with | 16 are | | |
| Spacers at | substituted with | | |
| positions 8 and | spacers | | |
| 16 (STMN2 | | | |
| AON variant | | | |
| 21_sp8sp16) | | | |
| STMN2 Variant | Nucleosides at | 1583 | XXXXXS₆XXXXXXXS₁₄XXXXXXX |
| Oligonucleotide | positions 6 and | | |
| (21 mer) with | 14 are | | |
| Spacers at | substituted with | | |
| positions 6 and | spacers | | |
| 14 (STMN2 | | | |
| AON variant | | | |
| 21_sp6sp14) | | | |
| STMN2 Variant | Nucleosides at | 1584 | XXXXXXXS₈XXXXXS₁₄XXXXXXX |
| Oligonucleotide | positions 8 and | | |
| (21 mer) with | 14 are | | |
| Spacers at | substituted with | | |
| positions 8 and | spacers | | |
| 14 (STMN2 | | | |
| AON variant | | | |
| 21_sp8sp14) | | | |
| STMN2 Variant | Nucleosides at | 1585 | XXXXXS₆XXXXXXXXXXXXXS₂₀X |
| Oligonucleotide | positions 6 and | | |
| (21 mer) with | 20 are | | |
| Spacers at | substituted with | | |
| positions 8 and | spacers | | |
| 14 (STMN2 | | | |
| AON variant | | | |
| 21_sp8sp14) | | | |
| STMN2 | N/A | 1586 | XXXXXXXXXXXXXXXXXXX |
| oligonucleotide | | | (19 mer) |
| variant (19 mer) | | | |
| STMN2 Variant | Nucleosides at | 1587 | XXXXS₅XXXXXXS₁₂XXXXXXX |
| Oligonucleotide | positions 5 and | | |
| (19 mer) with | 12 are | | |
| Spacers at | substituted with | | |
| positions 5 and | spacers | | |
| 12 (STMN2 | | | |
| AON variant | | | |
| 19_sp5sp12) | | | |
| STMN2 Variant | Nucleosides at | 1588 | XXXXXXXS₈XXXXXXS₁₅XXXX |
| Oligonucleotide | positions 8 and | | |
| (19 mer) with | 15 are | | |
| Spacers at | substituted with | | |
| positions 8 and | spacers | | |
| 15 (STMN2 | | | |
| AON variant | | | |
| 19_sp8sp15) | | | |

*At least one nucleoside linkage of the nucleobase sequence is selected from a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

In some embodiments, an antisense oligonucleotide disclosed herein (e.g., STMN2 parent oligonucleotides and/or STMN2 oligonucleotide variants) comprise one or more spacers as well as one or more locked nucleic acids (LNAs). In some embodiments, an antisense oligonucleotide disclosed herein (e.g., STMN2 parent oligonucleotides and/or STMN2 oligonucleotide variants) comprises two spacers and two LNAs. In some embodiments, an antisense oligonucleotide disclosed herein (e.g., STMN2 parent oligonucleotides and/or STMN2 oligonucleotide variants) comprises two spacers and three LNAs.

In various embodiments, a spacer and a LNA are located adjacent to one another in an antisense oligonucleotide. For example, if counting from 5′ to 3′, a LNA can be located at a 7^{th} position of the antisense oligonucleotide and a spacer can be located at a 8^{th} position of the antisense oligonucleotide. As another example, if counting from 5′ to 3′, a LNA can be located at a 9^{th} position of the antisense oligonucleotide and a spacer can be located at a 8^{th} position of the antisense oligonucleotide. As another example, if counting from 5′ to 3′, a LNA can be located at a 15^{th} position of the antisense oligonucleotide and a spacer can be located at a 16^{th} position of the antisense oligonucleotide. As another example, if counting from 5′ to 3′, a LNA can be located at a 17^{th} position of the antisense oligonucleotide and a spacer can be located at a 16^{th} position of the antisense oligonucleotide.

In particular embodiments, a first spacer is located adjacent to a first LNA and a second spacer is located adjacent to a second LNA in an antisense oligonucleotide. For example, if counting from 5′ to 3′, a first LNA can be located at a 7^{th} position of the antisense oligonucleotide, a first spacer can be located at a 8^{th} position of the antisense oligonucleotide, a second LNA can be located at a 15^{th} position of the antisense oligonucleotide, and a second spacer can be located at a 16^{th} position of the antisense oligonucleotide. As another example, if counting from 5′ to 3′, a first LNA can be located at a 7^{th} position of the antisense oligonucleotide, a first spacer can be located at a 8^{th} position of the antisense oligonucleotide, a second LNA can be located at a 17^{th} position of the antisense oligonucleotide, and a second spacer can be located at a 16^{th} position of the antisense oligonucleotide. As another example, if counting from 5′ to 3′, a first LNA can be located at a 9^{th} position of the antisense oligonucleotide, a first spacer can be located at a 8^{th} position of the antisense oligonucleotide, a second LNA can be located at a 17^{th} position of the antisense oligonucleotide, and a second spacer can be located at a 16^{th} position of the antisense oligonucleotide.

In various embodiments, one or more spacers and one or more LNAs are not located adjacent to one another in an antisense oligonucleotide. For example, if counting from 5′ to 3′, a LNA can be located at a 4^{th} position of the antisense oligonucleotide and a spacer can be located at a 8^{th} position of the antisense oligonucleotide. As example, if counting from 5′ to 3′, a LNA can be located at a 20^{th} position of the antisense oligonucleotide and a spacer can be located at a 16^{th} position of the antisense oligonucleotide. In particular embodiments, if counting from 5′ to 3′, a first LNA can be located at a 4^{th} position of the antisense oligonucleotide, a first spacer can be located at a 8^{th} position of the antisense oligonucleotide, a second LNA can be located at a 20^{th} position of the antisense oligonucleotide, and a second spacer can be located at a 16^{th} position of the antisense oligonucleotide. In particular embodiments, if counting from 5′ to 3′, a first LNA can be located at a 4^{th} position of the antisense oligonucleotide, a first spacer can be located at a 8^{th} position of the antisense oligonucleotide, a second LNA can be located at a 12^{th} position of the antisense oligonucleotide, a second spacer can be located at a 16^{th} position of the antisense oligonucleotide, and a third LNA can be located at a 20^{th} position of the antisense oligonucleotide.

### KCNQ2

As described further below, a KCNQ2 oligonucleotide with one or more spacers is described in reference to a corresponding KCNQ2 parent oligonucleotide or a KCNQ2 variant oligonucleotide. In various embodiments, a KCNQ2 oligonucleotide with a spacer differs from a KCNQ2 parent oligonucleotide or a KCNQ2 variant oligonucleotide in that the spacer replaces a nucleoside in the KCNQ2 parent oligonucleotide or a KCNQ2 variant oligonucleotide. As used hereafter, the “position” of the KCNQ2 oligonucleotide refers to a particular location as counted from the 5′ end of the KCNQ2 oligonucleotide. In various embodiments, the spacer replaces a nucleoside at any one of positions 1, 2, 3, 4, 5, 6, 7, 8, 9, 10, 11, 12, 13, 14, 15, 16, 17, 18, 19, 20, 21, 22, 23, 24, or 25 of the KCNQ2 parent oligonucleotide or a KCNQ2 variant oligonucleotide. In particular embodiments, a spacer replaces a nucleoside at one of positions 7, 8, 11, 14, 16, 19, or 22 of the KCNQ2 parent oligonucleotide or a KCNQ2 variant oligonucleotide.

In various embodiments, a KCNQ2 oligonucleotide includes one spacer that replaces a nucleoside in the KCNQ2 oligonucleotide (e.g., one spacer replaces one nucleoside of the KCNQ2 oligonucleotide). In particular embodiments, the spacer replaces a nucleoside between positions 9 and 15 of the KCNQ2 oligonucleotide. In particular embodiments, the spacer replaces a nucleoside between positions 9 and 12 of the KCNQ2 oligonucleotide. In particular embodiments, the spacer replaces a nucleoside at position 10 of the KCNQ2 oligonucleotide. In particular embodiments, the spacer replaces a nucleoside at position 11 of the KCNQ2 oligonucleotide. In particular embodiments, the spacer replaces a nucleoside at position 12 of the KCNQ2 oligonucleotide. In particular embodiments, the spacer replaces a nucleoside between positions 12 and 16 of the KCNQ2 oligonucleotide. In particular embodiments, the spacer replaces a nucleoside at position 15 of the KCNQ2 oligonucleotide.

In various embodiments, a KCNQ2 oligonucleotide including one spacer has 2 segments, where at least one of the 2 segments has at most 11 linked nucleosides. For example, the KCNQ2 oligonucleotide may be 23 oligonucleotide units in length, and the spacer can be located at position 12. Therefore, the KCNQ2 oligonucleotide has 2 segments divided by the spacer, where both of the 2 segments are 11 nucleobases in length. In various embodiments, a KCNQ2 oligonucleotide including one spacer has 2 segments, where at least one of the 2 segments has at most 10 linked nucleosides. For example, the KCNQ2 oligonucleotide may be 21 oligonucleotide units in length, and the spacer can be located at position 11. Therefore, the KCNQ2 oligonucleotide has 2 segments divided by the spacer, where both of the 2 segments are 10 nucleobases in length. As another example, the KCNQ2 oligonucleotide may be 25 oligonucleotide units in length, and the spacer can be located at position 15. Therefore, the KCNQ2 oligonucleotide has 2 segments divided by the spacer, where one of the 2 segments is 14 nucleobases in length and the second of the 2 segments is 10 nucleobases in length.

In various embodiments, a KCNQ2 oligonucleotide includes two spacers that each replace a nucleoside in the KCNQ2 oligonucleotide (e.g., two spacers replace two separate nucleosides of the KCNQ2 oligonucleotide). In various embodiments, a first spacer and a second spacer are separated by at least 5 nucleobases, at least 6 nucleobases, at least 7 nucleobases, at least 8 nucleobases, at least 9 nucleobases, or at least 10 nucleobases in the oligonucleotide. In particular embodiments, a first spacer and a second spacer are separated by at least 5 nucleobases, at least 6 nucleobases, or at least 7 nucleobases. In particular embodiments, the first spacer and the second spacer are not adjacent to one another in the oligonucleotide.

In particular embodiments, the first spacer replaces a nucleoside between positions 7 and 11 of the KCNQ2 oligonucleotide. In various embodiments, the first spacer replaces a nucleoside between positions 8 and 11, positions 9 and 11, positions 10 and 11, positions 7 and 10, positions 7 and 9, positions 7 and 8, positions 8 and 10, positions 8 and 9, or positions 9 and 10 of the KCNQ2 oligonucleotide. In particular embodiments, the second spacer replaces a nucleoside between positions 14 and 22 of the KCNQ2 oligonucleotide. In various embodiments, the second spacer replaces a nucleoside between positions 15 and 22, positions 16 and 22, positions 17 and 22, position 18 and 22, position 19 and 22, positions 20 and 22, positions 21 and 22, positions 15 and 21, position 16 and 21, positions 17 and 21, positions 18 and 21, positions 19 and 21, positions 20 and 21, positions 15 and 20, positions 16 and 20, positions 17 and 20, positions 18 and 20, positions 19 and 20, positions 15 and 19, positions 16 and 19, positions 17 and 19, positions 18 and 19, positions 15 and 18, position 16 and 18, position 17 and 18, positions 15 and 17, positions 16 and 17, or positions 15 and 16 of the KCNQ2 oligonucleotide.

In preferred embodiments, the first spacer replaces a nucleoside at position 7 of the KCNQ2 oligonucleotide and the second spacer replaces a nucleoside at position 14 of the KCNQ2 oligonucleotide. In preferred embodiments, the first spacer replaces a nucleoside at position 7 of the KCNQ2 oligonucleotide and the second spacer replaces a nucleoside at position 19 of the KCNQ2 oligonucleotide. In preferred embodiments, the first spacer replaces a nucleoside at position 8 of the KCNQ2 oligonucleotide and the second spacer replaces a nucleoside at position 16 of the KCNQ2 oligonucleotide. In preferred embodiments, the first spacer replaces a nucleoside at position 8 of the KCNQ2 oligonucleotide and the second spacer replaces a nucleoside at position 15 of the KCNQ2 oligonucleotide. In preferred embodiments, the first spacer replaces a nucleoside at position 11 of the KCNQ2 oligonucleotide and the second spacer replaces a nucleoside at position 22 of the KCNQ2 oligonucleotide. In preferred embodiments, the first spacer replaces a nucleoside at position 9 of the KCNQ2 oligonucleotide and the second spacer replaces a nucleoside at position 19 of the KCNQ2 oligonucleotide.

In various embodiments, a KCNQ2 oligonucleotide includes three spacers that each replace a nucleoside in the KCNQ2 oligonucleotide (e.g., three spacers replace three separate nucleosides of the KCNQ2 oligonucleotide). In particular embodiments, the first spacer replaces a nucleoside between positions 7 and 11 of the KCNQ2 oligonucleotide. In particular embodiments, the second spacer replaces a nucleoside between positions 14 and 22 of the KCNQ2 oligonucleotide. In particular embodiments, the third spacer replaces a nucleoside between positions 21 and 24 of the KCNQ2 oligonucleotide. In some embodiments, the first spacer replaces a nucleoside between positions 2 and 5 of the KCNQ2 oligonucleotide. In particular embodiments, the second spacer replaces a nucleoside between positions 8 and 12 of the KCNQ2 oligonucleotide. In particular embodiments, the third spacer replaces a nucleoside between positions 18 and 22 of the KCNQ2 oligonucleotide.

In various embodiments, the three spacers in a KCNQ2 oligonucleotide are positioned such that each of the four segments of the KCNQ2 oligonucleotide are at most 7 linked nucleosides in length. For example, a KCNQ2 oligonucleotide may have a first segment with 7 linked nucleosides connected to a first spacer, then a second segment with 7 linked nucleosides connected on one end to the first spacer and connected on another end to a second spacer, then a third segment with 6 linked nucleosides connected on one end to the second spacer and connected on another end to a third spacer, then a fourth segment with 6 linked nucleosides connected to the third spacer.

In various embodiments, the one or more spacers are positioned in the oligonucleotide to replace one or more adenosine or thymine nucleosides (as opposed to guanine or cytosine nucleosides). For example, the one or more spacers can replace one, two, three, four, five, six, seven, eight, or nine adenosine or thymine nucleosides in the oligonucleotide. In various embodiments, the one or more spacers are positioned in the oligonucleotide to replace one or more guanine or cytosine nucleosides (as opposed to adenosine or thymine nucleosides). For example, the one or more spacers can replace one, two, three, four, five, six, seven, eight, or nine guanine or cytosine nucleosides in the oligonucleotide. In various embodiments, the spacers are positioned in the oligonucleotide to replace an equal number of adenosine/thymine nucleosides and guanine/cytosine nucleosides. For example, a first spacer in the oligonucleotide may replace an adenosine/thymine nucleoside and a second spacer in the oligonucleotide may replace a guanine/cytosine nucleoside.

In various embodiments, the one or more spacers are positioned in the oligonucleotide to control the sequence content in the oligonucleotide. For example, the one or more spacers are positioned such that at least one of the spacers is located adjacent to a guanine group. In various embodiments, an oligonucleotide with spacers can include one spacer adjacent to a guanine group, two spacers adjacent to guanine groups, three spacers adjacent to guanine groups, four spacers adjacent to guanine groups, or five spacers adjacent to guanine groups. In one embodiment, if counting from the 5′ end of the oligonucleotide, a spacer immediately precedes a guanine group in the sequence. Thus, in various embodiments, an oligonucleotide with spacers can include one spacer that immediately precedes a guanine group, two spacers that each immediately precede a guanine group, three spacers that each immediately precede a guanine group, four spacers that each immediately precede a guanine group, or five spacers that each immediately precede a guanine group. In one embodiment, if counting from the 5′ end of the oligonucleotide, a guanine group is immediately succeeded by a spacer. Thus, in various embodiments, an oligonucleotide with spacers can include one spacer that immediately succeeds a guanine group, two spacers that each immediately succeed a guanine group, three spacers that each immediately succeed a guanine group, four spacers that each immediately succeed a guanine group, or five spacers that each immediately succeed a guanine group. In various embodiments, the spacers in the oligonucleotide can be positioned to maximize the number of spacers adjacent to guanine groups.

In various embodiments, the one or more spacers are positioned in the oligonucleotide to replace one or more adenosine or thymine nucleosides such that the one or more spacers are located adjacent guanine groups. For example, two spacers can replace adenosine or thymine nucleosides in the oligonucleotide, each of the two spacers being located adjacent to a guanine group.

In various embodiments, the KCNQ2 oligonucleotide with one or more spacers has a particular GC content. As used herein, GC content (or guanine-cytosine content) is the percentage of nitrogenous bases in the oligonucleotide that are either guanine (G) or cytosine (C). In various embodiments, the KCNQ2 oligonucleotide with one or more spacers has at least 10% GC content, at least 20% GC content, at least 25% GC content, at least 30% GC content, at least 35% GC content, at least 40% GC content, at least 45% GC content, at least 50% GC content, at least 55% GC content, at least 60% GC content, at least 65% GC content, at least 75% GC content, at least 80% GC content, at least 85% GC content, at least 90% GC content, or at least 95% GC content. In particular embodiments, the KCNQ2 oligonucleotide with one or more spacers has at least 30% GC content. In particular embodiments, the KCNQ2 oligonucleotide with one or more spacers has at least 40% GC content. In various embodiments, the one or more spacers are positioned in the KCNQ2 oligonucleotide to maximize GC content. For example, instead of selecting a guanine or cytosine for replacement by a spacer in the KCNQ2 oligonucleotide, a thymine or adenine can be selected for replacement by a spacer.

In various embodiments, a KCNQ2 oligonucleotide with spacers is designed such that 1) each segment of the KCNQ2 oligonucleotide has at most 7 linked nucleosides and 2) at least two, three, or four spacers are positioned adjacent to a guanine group. In some embodiments, a KCNQ2 oligonucleotide with spacers is designed such that 1) each segment of the KCNQ2 oligonucleotide has at most 7 linked nucleosides and 2) each of two spacers precede a guanine group.

In various embodiments, the inclusion of one or more spacers in the KCNQ2 oligonucleotide does not decrease the effectiveness of the KCNQ2 oligonucleotide with the spacers in restoring full length KCNQ2 protein or full length KCNQ2 mRNA in comparison to the effect of a corresponding KCNQ2 parent oligonucleotide. In various embodiments, the inclusion of one or more spacers in the KCNQ2 oligonucleotide increases the effectiveness of the KCNQ2 oligonucleotide with the spacers in restoring full length KCNQ2 protein or full length KCNQ2 mRNA in comparison to the effect of a corresponding KCNQ2 parent oligonucleotide. In various embodiments, the inclusion of one or more spacers in the KCNQ2 oligonucleotide does not decrease the effectiveness of the KCNQ2 oligonucleotide with the spacers in reducing quantity of KCNQ2 transcripts in comparison to the effect of a corresponding KCNQ2 parent oligonucleotide. In various embodiments, the inclusion of one or more spacers in the KCNQ2 oligonucleotide increases the effectiveness of the KCNQ2 oligonucleotide with the spacers in reducing quantity of KCNQ2 transcripts in comparison to the effect of a corresponding KCNQ2 parent oligonucleotide.

Tables 15A, 15B, 16, and 14 document example KCNQ2 oligonucleotides with one or more spacers and their relation to corresponding KCNQ2 parent oligonucleotides. Each KCNQ2 oligonucleotide is assigned a sequence name. As used hereafter, the nomenclature of the sequence name is expressed as “X_spA” (for a KCNQ2 AON with one spacer), “X_spA_spB” (for a KCNQ2 AON with two spacers), or “X_spA_spB_spC” (for a KCNQ2 AON with three spacers). Here, “X” refers to the length of the KCNQ2 AON, “A” refers to the position in the KCNQ2 AON where the first spacer is located, “B” refers to the position in the KCNQ2 AON where the second spacer is located, and if present, “C” refers to the position in the KCNQ2 AON where the third spacer is located.

In various embodiments, KCNQ2 oligonucleotides include one spacer. In various embodiments, the KCNQ2 oligonucleotides are oligonucleotide variants, such as any one of a 23mer, 21mer, or 19mer. In various embodiments, the inclusion of a spacer divides up the KCNQ2 oligonucleotide into two separate segments, where at least one of the segments is at most 11 linked nucleosides in length. In various embodiments, the inclusion of a spacer divides up the KCNQ2 oligonucleotide into two separate segments, where at least one of the segments is at most 10 linked nucleosides in length.

In various embodiments, the spacer is located between positions 10 and 15 of the oligonucleotide. In various embodiments, the spacer is located between positions 10 and 12 of the oligonucleotide. In particular embodiments, the spacer is located at position 10 of the oligonucleotide. In particular embodiments, the spacer is located at position 11 of the oligonucleotide. In particular embodiments, the spacer is located at position 12 of the oligonucleotide. In particular embodiments, the spacer is located at position 15 of the oligonucleotide. Example KCNQ2 AONs with one spacer are documented below in Table 15A.

TABLE 15A
Identification of KCNQ2 AONs with one spacer. Here, each KCNQ2 AON has 2
segments, where at least one of the segments has at most 11 linked nucleosides.

| | | | Sequence* (where X indicates a |
|---|---|---|---|
| | Relation to | Sequence | nucleoside of the KCNQ2 parent |
| | KCNQ2 | ID Number | oligonucleotide and S_{y} indicates |
| | oligonucleotide | (SEQ ID | presence of a Spacer where y |
| Sequence name | variant | NO) | denotes the position) (5′→3′) |
| KCNQ2 parent | N/A | 4402 | XXXXXXXXXXXXXXXXXXXXXXXXX |
| oligonucleotide | | | (25 mer) |
| KCNQ2 Oligonucleotide | Nucleoside at | 4403 | XXXXXXXXXXXXXXS₁₅XXXXXXXXXX |
| (25 mer) with Spacer at | position 15 of | | |
| position 15 | 25 mer is | | |
| (KCNQ2 AON 25_sp15) | substituted with | | |
| | a spacer | | |
| KCNQ2 oligonucleotide | N/A | 444 | XXXXXXXXXXXXXXXXXXXXXXX |
| variant (23 mer) | | | (23 mer) |
| KCNQ2 Oligonucleotide | Nucleoside at | 4405 | XXXXXXXXXXXS₁₂XXXXXXXXXXX |
| (23 mer) with Spacer at | position 12 of | | |
| position 12 | 23 mer is | | |
| (KCNQ2 AON 23_sp12) | substituted with | | |
| | a spacer | | |
| KCNQ2 oligonucleotide | N/A | 4406 | XXXXXXXXXXXXXXXXXXXXX |
| variant (21 mer) | | | (21 mer) |
| KCNQ2 Oligonucleotide | Nucleoside at | 4407 | XXXXXXXXXXS₁₁XXXXXXXXXX |
| (21 mer) with Spacer at | position 11 of | | |
| position 11 | 21 mer is | | |
| (KCNQ2 AON 21_sp11) | substituted with | | |
| | a spacer | | |
| KCNQ2 oligonucleotide | N/A | 4408 | XXXXXXXXXXXXXXXXXXX |
| variant (19 mer) | | | (19 mer) |
| KCNQ2 Oligonucleotide | Nucleoside at | 4409 | XXXXXXXXXS₁₀XXXXXXXXX |
| (19 mer) with Spacer at | position 10 of | | |
| position 10 | 19 mer is | | |
| (KCNQ2 AON 19_sp10) | substituted with | | |
| | a spacer | | |

*At least one nucleoside linkage of the nucleobase sequence is selected from a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

In various embodiments, KCNQ2 oligonucleotides include two spacers. In various embodiments, the inclusion of a spacer divides up the KCNQ2 oligonucleotide into three separate segments, where at least one of the segments is at most 7 linked nucleosides in length. Example KCNQ2 AONs with two spacers are documented below in Table 15B.

TABLE 15B
Identification of KCNQ2 AONs with two spacers. Here, each KCNQ2 AON has 3
segments, where at least one of the segments has at most 7 linked nucleosides.

| | | | Sequence* (where X indicates a |
|---|---|---|---|
| | | Sequence | nucleoside of the KCNQ2 parent |
| | Relation to | ID Number | oligonucleotide and S_{y} indicates |
| | KCNQ2 parent | (SEQ ID | presence of a Spacer where y |
| Sequence name | oligonucleotide | NO) | denotes the position) (5′→3′) |
| KCNQ2 parent | N/A | 4410 | XXXXXXXXXXXXXXXXXXXXXXXXX |
| oligonucleotide | | | |
| KCNQ2 | Nucleosides at | 4411 | XXXXXXXXXXS₁₁XXXXXXXXXXS₂₂XXX |
| Oligonucleotide | positions 11 and | | |
| with Spacers at | 22 are each | | |
| positions 11 and 22 | substituted with | | |
| (KCNQ2 AON | a spacer | | |
| 25_sp11sp22) | | | |
| KCNQ2 | Nucleosides at | 4412 | XXXXXXS₇XXXXXXS₁₄XXXXXXXXXXX |
| Oligonucleotide | positions 7 and | | |
| with Spacers at | 14 are each | | |
| positions 7 and 14 | substituted with | | |
| (KCNQ2 AON | a spacer | | |
| 25_sp7sp14) | | | |
| KCNQ2 | Nucleosides at | 4413 | XXXXXXXS₈XXXXXXXXXXS₁₉XXXXXX |
| Oligonucleotide | positions 8 and | | |
| with Spacers at | 19 are | | |
| positions 8 and 19 | substituted with | | |
| (KCNQ2 AON | spacers | | |
| 25_sp8sp19) | | | |
| KCNQ2 | Nucleosides at | 4414 | XXXXXXXS₈XXXXXS₁₄XXXXXXXXXXX |
| Oligonucleotide | positions 8 and | | |
| with Spacers at | 14 are | | |
| positions 8 and 14 | substituted with | | |
| (KCNQ2 AON | spacers | | |
| 25_sp8sp14) | | | |
| KCNQ2 | Nucleosides at | 4415 | XXXXXXXXS₉XXXXS₁₄XXXXXXXXXXX |
| Oligonucleotide | positions 9 and | | |
| with Spacers at | 14 are | | |
| positions 9 and 14 | substituted with | | |
| (KCNQ2 AON | spacers | | |
| 25_sp9sp14) | | | |
| KCNQ2 | Nucleosides at | 4416 | XXXXXXXXXS₁₀XXXS₁₄XXXXXXXXXXX |
| Oligonucleotide | positions 10 and | | |
| with Spacers at | 14 are | | |
| positions 10 and 14 | substituted with | | |
| (KCNQ2 AON | spacers | | |
| 25_sp10sp14) | | | |
| KCNQ2 | Nucleosides at | 4417 | XXXXXXXXXXS₁₁XXS₁₄XXXXXXXXXXX |
| Oligonucleotide | positions 11 and | | |
| with Spacers at | 14 are | | |
| positions 11 and 14 | substituted with | | |
| (KCNQ2 AON | spacers | | |
| 25_sp11sp14) | | | |
| KCNQ2 | Nucleosides at | 4418 | XXXXXXXS₈XXXXXXS₁₅XXXXXXXXXX |
| Oligonucleotide | positions 8 and | | |
| with Spacers at | 15 are | | |
| positions 8 and 15 | substituted with | | |
| (KCNQ2 AON | spacers | | |
| 25_sp8sp15) | | | |
| KCNQ2 | Nucleosides at | 4419 | XXXXXXXXS₉XXXXXS₁₅XXXXXXXXXX |
| Oligonucleotide | positions 9 and | | |
| with Spacers at | 15 are | | |
| positions 9 and 15 | substituted with | | |
| (KCNQ2 AON | spacers | | |
| 25_sp9sp15) | | | |
| KCNQ2 | Nucleosides at | 4420 | XXXXXXXXXS₁₀XXXXS₁₅XXXXXXXXXX |
| Oligonucleotide | positions 10 and | | |
| with Spacers at | 15 are | | |
| positions 10 and 15 | substituted with | | |
| (KCNQ2 AON | spacers | | |
| 25_sp10sp15) | | | |
| KCNQ2 | Nucleosides at | 4421 | XXXXXXXXXXS₁₁XXXS₁₅XXXXXXXXXX |
| Oligonucleotide | positions 11 and | | |
| with Spacers at | 15 are | | |
| positions 11 and 15 | substituted with | | |
| (KCNQ2 AON | spacers | | |
| 25_sp11sp15) | | | |
| KCNQ2 | Nucleosides at | 4422 | XXXXXXXS₈XXXXXXXS₁₆XXXXXXXXX |
| Oligonucleotide | positions 8 and | | |
| with Spacers at | 16 are | | |
| positions 8 and 16 | substituted with | | |
| (KCNQ2 AON | spacers | | |
| 25_sp8sp16) | | | |
| KCNQ2 | Nucleosides at | 4423 | XXXXXXXXS₉XXXXXXS₁₆XXXXXXXXX |
| Oligonucleotide | positions 9 and | | |
| with Spacers at | 16 are | | |
| positions 9 and 16 | substituted with | | |
| (KCNQ2 AON | spacers | | |
| 25_sp9sp16) | | | |
| KCNQ2 | Nucleosides at | 4424 | XXXXXXXXXS₁₀XXXXXS₁₆XXXXXXXXX |
| Oligonucleotide | positions 10 and | | |
| with Spacers at | 16 are | | |
| positions 10 and 16 | substituted with | | |
| (KCNQ2 AON | spacers | | |
| 25_sp10sp16) | | | |
| KCNQ2 | Nucleosides at | 4425 | XXXXXXXXXXS₁₁XXXXS₁₆XXXXXXXXX |
| Oligonucleotide | positions 11 and | | |
| with Spacers at | 16 are | | |
| positions 11 and 16 | substituted with | | |
| (KCNQ2 AON | spacers | | |
| 25_sp11sp16) | | | |
| KCNQ2 | Nucleosides at | 4426 | XXXXXXXS₈XXXXXXXXS₁₇XXXXXXXX |
| Oligonucleotide | positions 8 and | | |
| with Spacers at | 17 are | | |
| positions 8 and 17 | substituted with | | |
| (KCNQ2 AON | spacers | | |
| 25_sp8sp17) | | | |
| KCNQ2 | Nucleosides at | 4427 | XXXXXXXXS₉XXXXXXXS₁₇XXXXXXXX |
| Oligonucleotide | positions 9 and | | |
| with Spacers at | 17 are | | |
| positions 9 and 17 | substituted with | | |
| (KCNQ2 AON | spacers | | |
| 25_sp9sp17) | | | |
| KCNQ2 | Nucleosides at | 4428 | XXXXXXXXXS₁₀XXXXXXS₁₇XXXXXXXX |
| Oligonucleotide | positions 10 and | | |
| with Spacers at | 17 are | | |
| positions 10 and 17 | substituted with | | |
| (KCNQ2 AON | spacers | | |
| 25_sp10sp17) | | | |
| KCNQ2 | Nucleosides at | 4429 | XXXXXXXXXXS₁₁XXXXXS₁₇XXXXXXXX |
| Oligonucleotide | positions 11 and | | |
| with Spacers at | 17 are | | |
| positions 11 and 17 | substituted with | | |
| (KCNQ2 AON | spacers | | |
| 25_sp11sp17) | | | |
| KCNQ2 | Nucleosides at | 4430 | XXXXXXXS₈XXXXXXXXXS₁₈XXXXXXX |
| Oligonucleotide | positions 8 and | | |
| with Spacers at | 18 are | | |
| positions 8 and 18 | substituted with | | |
| (KCNQ2 ATON | spacers | | |
| 25_sp8sp18) | | | |
| KCNQ2 | Nucleosides at | 4431 | XXXXXXXXS₉XXXXXXXXS₁₈XXXXXXX |
| Oligonucleotide | positions 9 and | | |
| with Spacers at | 18 are | | |
| positions 9 and 18 | substituted with | | |
| (KCNQ2 AON | spacers | | |
| 25_sp9sp18) | | | |
| KCNQ2 | Nucleosides at | 4432 | XXXXXXXXXS₁₀XXXXXXXS₁₈XXXXXXX |
| Oligonucleotide | positions 10 and | | |
| with Spacers at | 18 are | | |
| positions 10 and 18 | substituted with | | |
| (KCNQ2 AON | spacers | | |
| 25_sp10sp18) | | | |
| KCNQ2 | Nucleosides at | 4433 | XXXXXXXXXXS₁₁XXXXXXS₁₈XXXXXXX |
| Oligonucleotide | positions 11 and | | |
| with Spacers at | 18 are | | |
| positions 11 and 18 | substituted with | | |
| (KCNQ2 AON | spacers | | |
| 25_sp11sp18) | | | |
| KCNQ2 | Nucleosides at | 4434 | XXXXXXXXS₉XXXXXXXXXS₁₉XXXXXX |
| Oligonucleotide | positions 9 and | | |
| with Spacers at | 19 are | | |
| positions 9 and 19 | substituted with | | |
| (KCNQ2 AON | spacers | | |
| 25_sp9sp19) | | | |
| KCNQ2 | Nucleosides at | 4435 | XXXXXXXXXS₁₀XXXXXXXXS₁₉XXXXXX |
| Oligonucleotide | positions 10 and | | |
| with Spacers at | 19 are | | |
| positions 10 and 19 | substituted with | | |
| (KCNQ2 AON | spacers | | |
| 25_sp10sp19) | | | |
| KCNQ2 | Nucleosides at | 4436 | XXXXXXXXXXS₁₁XXXXXXXS₁₉XXXXXX |
| Oligonucleotide | positions 11 and | | |
| with Spacers at | 19 are | | |
| positions 11 and 19 | substituted with | | |
| (KCNQ2 AON | spacers | | |
| 25_sp11sp19) | | | |
| KCNQ2 | Nucleosides at | 4437 | XXXXXXXXS₉XXXXXXXXXXS₂₀XXXXX |
| Oligonucleotide | positions 9 and | | |
| with Spacers at | 20 are | | |
| positions 9 and 20 | substituted with | | |
| (KCNQ2 AON | spacers | | |
| 25_sp9sp20) | | | |
| KCNQ2 | Nucleosides at | 4438 | XXXXXXXXXS₁₀XXXXXXXXXS₂₀XXXXX |
| Oligonucleotide | positions 10 and | | |
| with Spacers at | 20 are | | |
| positions 10 and 20 | substituted with | | |
| (KCNQ2 AON | spacers | | |
| 25_sp10sp20) | | | |
| KCNQ2 | Nucleosides at | 4439 | XXXXXXXXXXS₁₁XXXXXXXXS₂₀XXXXX |
| Oligonucleotide | positions 11 and | | |
| with Spacers at | 20 are | | |
| positions 11 and 20 | substituted with | | |
| (KCNQ2 AON | spacers | | |
| 25_sp11sp20) | | | |
| KCNQ2 | Nucleosides at | 4440 | XXXXXXXXXS₁₀XXXXXXXXXXS₂₁XXXX |
| Oligonucleotide | positions 10 and | | |
| with Spacers at | 21 are | | |
| positions 10 and 21 | substituted with | | |
| (KCNQ2 AON | spacers | | |
| 25_sp10sp21) | | | |
| KCNQ2 | Nucleosides at | 4441 | XXXXXXXXXXS₁₁XXXXXXXXXS₂₁XXXX |
| Oligonucleotide | positions 11 and | | |
| with Spacers at | 21 are | | |
| positions 11 and 21 | substituted with | | |
| (KCNQ2 AON | spacers | | |
| 25_sp11sp21) | | | |
| KCNQ2 | Nucleosides at | 4442 | XXXS₄XXXXXXXXXXS₁₅XXXXXXXXXX |
| Oligonucleotide | positions 4 and | | |
| with Spacers at | 15 are | | |
| positions 4 and 15 | substituted with | | |
| (KCNQ2 AON | spacers | | |
| 25_sp4sp15) | | | |
| KCNQ2 | Nucleosides at | 4443 | XXXXXXS₇XXXXXXXXXXXS₁₉XXXXXX |
| Oligonucleotide | positions 7 and | | |
| with Spacers at | 19 are | | |
| positions 7 and 19 | substituted with | | |
| (KCNQ2 AON | spacers | | |
| 25_sp7sp19) | | | |
| KCNQ2 | Nucleosides at | 4444 | XXXXXXS₇XXXXXXXXXXS₁₈XXXXXXX |
| Oligonucleotide | positions 7 and | | |
| with Spacers at | 18 are | | |
| positions 7 and 18 | substituted with | | |
| (KCNQ2 AON | spacers | | |
| 25_sp7sp18) | | | |
| KCNQ2 | Nucleosides at | 4445 | XXXXXXXXS₉XXXXXXXXXXXS₂₁XXXX |
| Oligonucleotide | positions 9 and | | |
| with Spacers at | 21 are | | |
| positions 9 and 21 | substituted with | | |
| (KCNQ2 AON | spacers | | |
| 25_sp9sp21) | | | |

*At least one nucleoside linkage of the nucleobase sequence is selected from a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

In various embodiments, KCNQ2 oligonucleotides include three spacers. The inclusion of three spacers divides up the KCNQ2 oligonucleotide into four separate segments. In various embodiments, the three spacers are located at different positions of the KCNQ2 oligonucleotide such that each of the segments of the KCNQ2 oligonucleotide are at most 7 linked nucleosides in length. Example KCNQ2 AONs with three spacers are documented below in Table 16.

TABLE 16
Identification of KCNQ2 AONs or AON variants with three spacers. Here, each
KCNQ2 AON has 4 segments, where each segment has at most 7 linked nucleosides.

| | | | Sequence* (where X indicates a |
|---|---|---|---|
| | Relation to | | nucleoside of the KCNQ2 parent |
| | KCNQ2 | Sequence ID | oligonucleotide and S_{y} indicates |
| | parent | Number | presence of a Spacer where y denotes |
| Sequence name | oligonucleotide | (SEQ ID NO) | the position) (5′→3′) |
| KCNQ2 | Nucleosides | 4446 | XXXXXXXS₈XXXXXXXS₁₆XXXXXXXS₂₄X |
| Oligonucleotide | at positions 8 | | |
| with Spacers at | and 16 and 24 | | |
| positions 8 and 16 | are substituted | | |
| and 24 (KCNQ2 | with spacers | | |
| AON | | | |
| 25_sp8sp16sp24) | | | |
| KCNQ2 | Nucleosides | 4447 | XXXXXXXS₈XXXXXXXS₁₆XXXXXXS₂₃XX |
| Oligonucleotide | at positions 8 | | |
| with Spacers at | and 16 and 23 | | |
| positions 8 and 16 | are substituted | | |
| and 23 (KCNQ2 | with spacers | | |
| AON | | | |
| 25_sp8sp16sp23) | | | |
| KCNQ2 | Nucleosides | 4448 | XS₂XXXXXXXS₁₀XXXXXXXS₁₈XXXXXXX |
| Oligonucleotide | at positions 2 | | |
| with Spacers at | and 10 and 18 | | |
| positions 2 and 10 | are substituted | | |
| and 18 (KCNQ2 | with spacers | | |
| AON | | | |
| 25_sp8sp16sp23) | | | |
| KCNQ2 | Nucleosides | 4449 | XXS₃XXXXXXS₁₀XXXXXXXS₁₈XXXXXXX |
| Oligonucleotide | at positions 3 | | |
| with Spacers at | and 10 and 18 | | |
| positions 3 and 10 | are substituted | | |
| and 18 (KCNQ2 | with spacers | | |
| AON | | | |
| 25_sp8sp16sp23) | | | |
| KCNQ2 | Nucleosides | 4450 | XXXS₄XXXXXXXS₁₂XXXXXXS₁₉XXXXXX |
| Oligonucleotide | at positions 4 | | |
| with Spacers at | and 12 and 19 | | |
| positions 4 and 12 | are substituted | | |
| and 19 (KCNQ2 | with spacers | | |
| AON | | | |
| 25_sp4sp12sp19) | | | |
| KCNQ2 | Nucleosides | 4451 | XXXXXXXS₈XXXXS₁₃XXXXS₁₈XXXXXXX |
| Oligonucleotide | at positions 8 | | |
| with Spacers at | and 13 and 18 | | |
| positions 8 and 13 | are substituted | | |
| and 18 (KCNQ2 | with spacers | | |
| AON | | | |
| 25_sp8sp13sp18) | | | |
| KCNQ2 | Nucleosides | 4452 | XXXXS₅XXXXXXXS₁₃XXXXXXXS₂₁XXXX |
| Oligonucleotide | at positions 5 | | |
| with Spacers at | and 13 and 21 | | |
| positions 5 and 13 | are substituted | | |
| and 21 (KCNQ2 | with spacers | | |
| AON | | | |
| 25_sp5sp13sp21) | | | |
| KCNQ2 | Nucleosides | 4453 | XXXXXXS₇XXXXXS₁₃XXXXXS₁₉XXXXXX |
| Oligonucleotide | at positions 7 | | |
| with Spacers at | and 13 and 19 | | |
| positions 7 and 13 | are substituted | | |
| and 19 (KCNQ2 | with spacers | | |
| AON | | | |
| 25_sp7sp13sp19) | | | |
| KCNQ2 | Nucleosides | 4454 | XXXXXS₆XXXXXXS₁₃XXXXXXS₂₀XXXXX |
| Oligonucleotide | at positions 6 | | |
| with Spacers at | and 13 and 20 | | |
| positions 6 and 13 | are substituted | | |
| and 20 (KCNQ2 | with spacers | | |
| AON | | | |
| 25_sp6sp13sp20) | | | |
| KCNQ2 | Nucleosides | 4455 | XXXXXXXS₈XXS₁₁XXXXXXXS₁₉XXXXXX |
| Oligonucleotide | at positions 8 | | |
| with Spacers at | and 11 and 19 | | |
| positions 8 and 11 | are substituted | | |
| and 19 (KCNQ2 | with spacers | | |
| AON | | | |
| 25_sp8sp11sp19) | | | |
| KCNQ2 | Nucleosides | 4456 | XXXXXXXS₈XXS₁₁XXXXS₁₆XXXXXXX |
| Oligonucleotide | at positions 8 | | |
| with Spacers at | and 11 and 16 | | |
| positions 8 and 11 | | | |
| and 16 (KCNQ2 | are substituted | | |
| AON | with spacers | | |
| 23_sp8sp11sp16) | | | |
| KCNQ2 | Nucleosides | 4457 | XXXXXXS₇XXXXXXS₁₄XXXXXXXS₂₂XXX |
| Oligonucleotide | at positions 7 | | |
| with Spacers at | and 14 and 22 | | |
| positions 7 and 14 | are substituted | | |
| and 22 (KCNQ2 | with spacers | | |
| AON | | | |
| 23_sp7sp14sp22) | | | |

*At least one nucleoside linkage of the nucleobase sequence is selected from a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

In various embodiments, KCNQ2 AONs with one or more spacers are reduced in length in comparison to the KCNQ2 AONs described above in Tables 15B and 16. For example, such KCNQ2 AONs may be KCNQ2 oligonucleotide variants with one or more spacers. In various embodiments, the KCNQ2 oligonucleotide variants with one or more spacers are 23mers, 21mers, or 19mers. In various embodiments, KCNQ2 oligonucleotide variants include two spacers such that the KCNQ2 oligonucleotide variant includes three segments that are divided up by the two spacers. In various embodiments, at least one of the three segments has at most 7 linked nucleosides. In various embodiments, each of the three segments has at most 7 linked nucleosides. Example KCNQ2 oligonucleotide variants with one or more spacers are shown below in Table 17.

TABLE 17
KCNQ2 AON variants with two spacers. Here, each KCNQ2 AON variant has 3
segments, where each segment has at most 7 linked nucleosides.

| | | | Sequence* (where X indicates a |
|---|---|---|---|
| | Relation to | | nucleoside of the KCNQ2 parent |
| | KCNQ2 | Sequence ID | oligonucleotide and S_{y} indicates |
| | oligonucleotide | Number | presence of a Spacer where y denotes |
| Sequence name | variant | (SEQ ID NO) | the position) (5′→3′) |
| KCNQ2 | N/A | 4458 | XXXXXXXXXXXXXXXXXXXXXXX |
| oligonucleotide | | | (23 mer) |
| variant (23 mer) | | | |
| KCNQ2 Variant | Nucleosides at | 4459 | XXXXXXXS₈XXXXXXXS₁₆XXXXXXX |
| Oligonucleotide | positions 8 and | | |
| (23 mer) with | 16 are | | |
| Spacers at | substituted with | | |
| positions 8 and | spacers | | |
| 16 (KCNQ2 | | | |
| AON variant | | | |
| 23_sp8sp16) | | | |
| KCNQ2 | N/A | 4460 | XXXXXXXXXXXXXXXXXXXXX |
| oligonucleotide | | | (21 mer) |
| variant (21 mer) | | | |
| KCNQ2 Variant | Nucleosides at | 4461 | XXXXS₅XXXXXXS₁₂XXXXXXXXX |
| Oligonucleotide | positions 5 and | | |
| (21 mer) with | 12 are | | |
| Spacers at | substituted with | | |
| positions 5 and | spacers | | |
| 12 (KCNQ2 | | | |
| AON variant | | | |
| 21_sp5sp12) | | | |
| KCNQ2 Variant | Nucleosides at | 4462 | XXXXXXXS₈XXXXXXXS₁₆XXXXX |
| Oligonucleotide | positions 8 and | | |
| (21 mer) with | 16 are | | |
| Spacers at | substituted with | | |
| positions 8 and | spacers | | |
| 16 (KCNQ2 | | | |
| AON variant | | | |
| 21_sp8sp16) | | | |
| KCNQ2 Variant | Nucleosides at | 4463 | XXXXXS₆XXXXXXXS₁₄XXXXXXX |
| Oligonucleotide | positions 6 and | | |
| (21 mer) with | 14 are | | |
| Spacers at | substituted with | | |
| positions 6 and | spacers | | |
| 14 (KCNQ2 | | | |
| AON variant | | | |
| 21_sp6sp14) | | | |
| KCNQ2 Variant | Nucleosides at | 4464 | XXXXXXXS₈XXXXXS₁₄XXXXXXX |
| Oligonucleotide | positions 8 and | | |
| (21 mer) with | 14 are | | |
| Spacers at | substituted with | | |
| positions 8 and | spacers | | |
| 14 (KCNQ2 | | | |
| AON variant | | | |
| 21_sp8sp14) | | | |
| KCNQ2 Variant | Nucleosides at | 4465 | XXXXXS₆XXXXXXXXXXXXXS₂₀X |
| Oligonucleotide | positions 6 and | | |
| (21 mer) with | 20 are | | |
| Spacers at | substituted with | | |
| positions 8 and | spacers | | |
| 14 (KCNQ2 | | | |
| AON variant | | | |
| 21_sp8sp14) | | | |
| KCNQ2 | N/A | 4466 | XXXXXXXXXXXXXXXXXXX |
| oligonucleotide | | | (19 mer) |
| variant (19 mer) | | | |
| KCNQ2 Variant | Nucleosides at | 4467 | XXXXS₅XXXXXXS₁₂XXXXXXX |
| Oligonucleotide | positions 5 and | | |
| (19 mer) with | 12 are | | |
| Spacers at | substituted with | | |
| positions 5 and | spacers | | |
| 12 (KCNQ2 | | | |
| AON variant | | | |
| 19_sp5sp12) | | | |
| KCNQ2 Variant | Nucleosides at | 4468 | XXXXXXXS₈XXXXXXS₁₅XXXX |
| Oligonucleotide | positions 8 and | | |
| (19 mer) with | 15 are | | |
| Spacers at | substituted with | | |
| positions 8 and | spacers | | |
| 15 (KCNQ2 | | | |
| AON variant | | | |
| 19_sp8sp15) | | | |

*At least one nucleoside linkage of the nucleobase sequence is selected from a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

In some embodiments, an antisense oligonucleotide disclosed herein (e.g., KCNQ2 parent oligonucleotides and/or KCNQ2 oligonucleotide variants) comprise one or more spacers as well as one or more locked nucleic acids (LNAs). In some embodiments, an antisense oligonucleotide disclosed herein (e.g., KCNQ2 parent oligonucleotides and/or KCNQ2 oligonucleotide variants) comprises two spacers and two LNAs. In some embodiments, an antisense oligonucleotide disclosed herein (e.g., KCNQ2 parent oligonucleotides and/or KCNQ2 oligonucleotide variants) comprises two spacers and three LNAs.

In various embodiments, a spacer and a LNA are located adjacent to one another in an antisense oligonucleotide. For example, if counting from 5′ to 3′, a LNA can be located at a 7^{th} position of the antisense oligonucleotide and a spacer can be located at a 8^{th} position of the antisense oligonucleotide. As another example, if counting from 5′ to 3′, a LNA can be located at a 9^{th} position of the antisense oligonucleotide and a spacer can be located at a 8^{th} position of the antisense oligonucleotide. As another example, if counting from 5′ to 3′, a LNA can be located at a 15^{th} position of the antisense oligonucleotide and a spacer can be located at a 16^{th} position of the antisense oligonucleotide. As another example, if counting from 5′ to 3′, a LNA can be located at a 17^{th} position of the antisense oligonucleotide and a spacer can be located at a 16^{th} position of the antisense oligonucleotide.

In particular embodiments, a first spacer is located adjacent to a first LNA and a second spacer is located adjacent to a second LNA in an antisense oligonucleotide. For example, if counting from 5′ to 3′, a first LNA can be located at a 7^{th} position of the antisense oligonucleotide, a first spacer can be located at a 8^{th} position of the antisense oligonucleotide, a second LNA can be located at a 15^{th} position of the antisense oligonucleotide, and a second spacer can be located at a 16^{th} position of the antisense oligonucleotide. As another example, if counting from 5′ to 3′, a first LNA can be located at a 7^{th} position of the antisense oligonucleotide, a first spacer can be located at a 8^{th} position of the antisense oligonucleotide, a second LNA can be located at a 17^{th} position of the antisense oligonucleotide, and a second spacer can be located at a 16^{th} position of the antisense oligonucleotide. As another example, if counting from 5′ to 3′, a first LNA can be located at a 9^{th} position of the antisense oligonucleotide, a first spacer can be located at a 8^{th} position of the antisense oligonucleotide, a second LNA can be located at a 17m position of the antisense oligonucleotide, and a second spacer can be located at a 16^{th} position of the antisense oligonucleotide.

In various embodiments, one or more spacers and one or more LNAs are not located adjacent to one another in an antisense oligonucleotide. For example, if counting from 5′ to 3′, a LNA can be located at a 4^{th} position of the antisense oligonucleotide and a spacer can be located at a 8^{th} position of the antisense oligonucleotide. As example, if counting from 5′ to 3′, a LNA can be located at a 20^{th} position of the antisense oligonucleotide and a spacer can be located at a 16^{th} position of the antisense oligonucleotide. In particular embodiments, if counting from 5′ to 3′, a first LNA can be located at a 4^{th} position of the antisense oligonucleotide, a first spacer can be located at a 8^{th} position of the antisense oligonucleotide, a second LNA can be located at a 20^{th} position of the antisense oligonucleotide, and a second spacer can be located at a 16^{th} position of the antisense oligonucleotide. In particular embodiments, if counting from 5′ to 3′, a first LNA can be located at a 4^{th} position of the antisense oligonucleotide, a first spacer can be located at a 8^{th} position of the antisense oligonucleotide, a second LNA can be located at a 12^{th} position of the antisense oligonucleotide, a second spacer can be located at a 16^{th} position of the antisense oligonucleotide, and a third LNA can be located at a 20^{th} position of the antisense oligonucleotide.

### UNC13A

As described further below, a UNC13A oligonucleotide with one or more spacers is described in reference to a corresponding UNC13A parent oligonucleotide or a corresponding UNC13A variant oligonucleotide. In various embodiments, a UNC13A oligonucleotide with a spacer differs from a UNC13A parent oligonucleotide or an UNC13A variant oligonucleotide in that the spacer replaces a nucleoside in the UNC13A parent oligonucleotide or an UNC13A variant oligonucleotide. As used hereafter, the “position” of the UNC13A oligonucleotide refers to a particular location as counted from the 5′ end of the UNC13A oligonucleotide. In various embodiments, the spacer replaces a nucleoside at any one of positions 1, 2, 3, 4, 5, 6, 7, 8, 9, 10, 11, 12, 13, 14, 15, 16, 17, 18, 19, 20, 21, 22, 23, 24, or 25 of the UNC13A parent oligonucleotide or an UNC13A variant oligonucleotide. In particular embodiments, a spacer replaces a nucleoside at one of positions 7, 8, 11, 14, 16, 19, or 22 of the UNC13A parent oligonucleotide or an UNC13A variant oligonucleotide.

In various embodiments, a UNC13A oligonucleotide includes one spacer that replaces a nucleoside in the UNC13A oligonucleotide (e.g., one spacer replaces one nucleoside of the UNC13A oligonucleotide). In particular embodiments, the spacer replaces a nucleoside between positions 9 and 15 of the UNC13A oligonucleotide. In particular embodiments, the spacer replaces a nucleoside between positions 9 and 12 of the UNC13A oligonucleotide. In particular embodiments, the spacer replaces a nucleoside at position 10 of the UNC13A oligonucleotide. In particular embodiments, the spacer replaces a nucleoside at position 11 of the UNC13A oligonucleotide. In particular embodiments, the spacer replaces a nucleoside at position 12 of the UNC13A oligonucleotide. In particular embodiments, the spacer replaces a nucleoside between positions 12 and 16 of the UNC13A oligonucleotide. In particular embodiments, the spacer replaces a nucleoside at position 15 of the UNC13A oligonucleotide.

In various embodiments, a UNC13A oligonucleotide including one spacer has 2 segments, where at least one of the 2 segments has at most 11 linked nucleosides. For example, the UNC13A oligonucleotide may be 23 oligonucleotide units in length, and the spacer can be located at position 12. Therefore, the UNC13A oligonucleotide has 2 segments divided by the spacer, where both of the 2 segments are 11 nucleobases in length. In various embodiments, a UNC13A oligonucleotide including one spacer has 2 segments, where at least one of the 2 segments has at most 10 linked nucleosides. For example, the UNC13A oligonucleotide may be 21 oligonucleotide units in length, and the spacer can be located at position 11. Therefore, the UNC13A oligonucleotide has 2 segments divided by the spacer, where both of the 2 segments are 10 nucleobases in length. As another example, the UNC13A oligonucleotide may be 25 oligonucleotide units in length, and the spacer can be located at position 15. Therefore, the UNC13A oligonucleotide has 2 segments divided by the spacer, where one of the 2 segments is 14 nucleobases in length and the second of the 2 segments is 10 nucleobases in length.

In various embodiments, a UNC13A oligonucleotide includes two spacers that each replace a nucleoside in the UNC13A oligonucleotide (e.g., two spacers replace two separate nucleosides of the UNC13A oligonucleotide). In various embodiments, a first spacer and a second spacer are separated by at least 5 nucleobases, at least 6 nucleobases, at least 7 nucleobases, at least 8 nucleobases, at least 9 nucleobases, or at least 10 nucleobases in the oligonucleotide. In particular embodiments, a first spacer and a second spacer are separated by at least 5 nucleobases, at least 6 nucleobases, or at least 7 nucleobases. In particular embodiments, the first spacer and the second spacer are not adjacent to one another in the oligonucleotide.

In particular embodiments, the first spacer replaces a nucleoside between positions 7 and 11 of the UNC13A oligonucleotide. In various embodiments, the first spacer replaces a nucleoside between positions 8 and 11, positions 9 and 11, positions 10 and 11, positions 7 and 10, positions 7 and 9, positions 7 and 8, positions 8 and 10, positions 8 and 9, or positions 9 and 10 of the UNC13A oligonucleotide. In particular embodiments, the second spacer replaces a nucleoside between positions 14 and 22 of the UNC13A oligonucleotide. In various embodiments, the second spacer replaces a nucleoside between positions 15 and 22, positions 16 and 22, positions 17 and 22, position 18 and 22, position 19 and 22, positions 20 and 22, positions 21 and 22, positions 15 and 21, position 16 and 21, positions 17 and 21, positions 18 and 21, positions 19 and 21, positions 20 and 21, positions 15 and 20, positions 16 and 20, positions 17 and 20, positions 18 and 20, positions 19 and 20, positions 15 and 19, positions 16 and 19, positions 17 and 19, positions 18 and 19, positions 15 and 18, position 16 and 18, position 17 and 18, positions 15 and 17, positions 16 and 17, or positions 15 and 16 of the UNC13A oligonucleotide.

In preferred embodiments, the first spacer replaces a nucleoside at position 7 of the UNC13A oligonucleotide and the second spacer replaces a nucleoside at position 14 of the UNC13A oligonucleotide. In preferred embodiments, the first spacer replaces a nucleoside at position 7 of the UNC13A oligonucleotide and the second spacer replaces a nucleoside at position 19 of the UNC13A oligonucleotide. In preferred embodiments, the first spacer replaces a nucleoside at position 8 of the UNC13A parent oligonucleotide and the second spacer replaces a nucleoside at position 16 of the UNC13A parent oligonucleotide. In preferred embodiments, the first spacer replaces a nucleoside at position 8 of the UNC13A oligonucleotide and the second spacer replaces a nucleoside at position 15 of the UNC13A oligonucleotide. In preferred embodiments, the first spacer replaces a nucleoside at position 11 of the UNC13A oligonucleotide and the second spacer replaces a nucleoside at position 22 of the UNC13A oligonucleotide. In preferred embodiments, the first spacer replaces a nucleoside at position 9 of the UNC13A oligonucleotide and the second spacer replaces a nucleoside at position 19 of the UNC13A oligonucleotide.

In various embodiments, a UNC13A oligonucleotide includes three spacers that each replace a nucleoside in the UNC13A oligonucleotide (e.g., three spacers replace three separate nucleosides of the UNC13A oligonucleotide). In particular embodiments, the first spacer replaces a nucleoside between positions 7 and 11 of the UNC13A oligonucleotide. In particular embodiments, the second spacer replaces a nucleoside between positions 14 and 22 of the UNC13A oligonucleotide. In particular embodiments, the third spacer replaces a nucleoside between positions 21 and 24 of the UNC13A oligonucleotide. In some embodiments, the first spacer replaces a nucleoside between positions 2 and 5 of the UNC13A oligonucleotide. In particular embodiments, the second spacer replaces a nucleoside between positions 8 and 12 of the UNC13A oligonucleotide. In particular embodiments, the third spacer replaces a nucleoside between positions 18 and 22 of the UNC13A oligonucleotide.

In various embodiments, the three spacers in a UNC13A oligonucleotide are positioned such that each of the four segments of the UNC13A oligonucleotide are at most 7 linked nucleosides in length. For example, a UNC13A oligonucleotide may have a first segment with 7 linked nucleosides connected to a first spacer, then a second segment with 7 linked nucleosides connected on one end to the first spacer and connected on another end to a second spacer, then a third segment with 6 linked nucleosides connected on one end to the second spacer and connected on another end to a third spacer, then a fourth segment with 6 linked nucleosides connected to the third spacer.

In various embodiments, the one or more spacers are positioned in the oligonucleotide to replace one or more adenosine or thymine nucleosides (as opposed to guanine or cytosine nucleosides). For example, the one or more spacers can replace one, two, three, four, five, six, seven, eight, or nine adenosine or thymine nucleosides in the oligonucleotide. In various embodiments, the one or more spacers are positioned in the oligonucleotide to replace one or more guanine or cytosine nucleosides (as opposed to adenosine or thymine nucleosides). For example, the one or more spacers can replace one, two, three, four, five, six, seven, eight, or nine guanine or cytosine nucleosides in the oligonucleotide. In various embodiments, the spacers are positioned in the oligonucleotide to replace an equal number of adenosine/thymine nucleosides and guanine/cytosine nucleosides. For example, a first spacer in the oligonucleotide may replace an adenosine/thymine nucleoside and a second spacer in the oligonucleotide may replace a guanine/cytosine nucleoside.

In various embodiments, the one or more spacers are positioned in the oligonucleotide to control the sequence content in the oligonucleotide. For example, the one or more spacers are positioned such that at least one of the spacers is located adjacent to a guanine group. In various embodiments, an oligonucleotide with spacers can include one spacer adjacent to a guanine group, two spacers adjacent to guanine groups, three spacers adjacent to guanine groups, four spacers adjacent to guanine groups, or five spacers adjacent to guanine groups. In one embodiment, if counting from the 5′ end of the oligonucleotide, a spacer immediately precedes a guanine group in the sequence. Thus, in various embodiments, an oligonucleotide with spacers can include one spacer that immediately precedes a guanine group, two spacers that each immediately precede a guanine group, three spacers that each immediately precede a guanine group, four spacers that each immediately precede a guanine group, or five spacers that each immediately precede a guanine group. In one embodiment, if counting from the 5′ end of the oligonucleotide, a guanine group is immediately succeeded by a spacer. Thus, in various embodiments, an oligonucleotide with spacers can include one spacer that immediately succeeds a guanine group, two spacers that each immediately succeed a guanine group, three spacers that each immediately succeed a guanine group, four spacers that each immediately succeed a guanine group, or five spacers that each immediately succeed a guanine group. In various embodiments, the spacers in the oligonucleotide can be positioned to maximize the number of spacers adjacent to guanine groups.

In various embodiments, the one or more spacers are positioned in the oligonucleotide to replace one or more adenosine or thymine nucleosides such that the one or more spacers are located adjacent guanine groups. For example, two spacers can replace adenosine or thymine nucleosides in the oligonucleotide, each of the two spacers being located adjacent to a guanine group.

In various embodiments, the UNC13A oligonucleotide with one or more spacers has a particular GC content. As used herein, GC content (or guanine-cytosine content) is the percentage of nitrogenous bases in the oligonucleotide that are either guanine (G) or cytosine (C). In various embodiments, the UNC13A oligonucleotide with one or more spacers has at least 10% GC content, at least 20% GC content, at least 25% GC content, at least 30% GC content, at least 35% GC content, at least 40% GC content, at least 45% GC content, at least 50% GC content, at least 55% GC content, at least 60% GC content, at least 65% GC content, at least 75% GC content, at least 80% GC content, at least 85% GC content, at least 90% GC content, or at least 95% GC content. In particular embodiments, the UNC13A oligonucleotide with one or more spacers has at least 30% GC content. In particular embodiments, the UNC13A oligonucleotide with one or more spacers has at least 40% GC content. In various embodiments, the one or more spacers are positioned in the UNC13A oligonucleotide to maximize GC content. For example, instead of selecting a guanine or cytosine for replacement by a spacer in the UNC13A oligonucleotide, a thymine or adenine can be selected for replacement by a spacer.

In various embodiments, a UNC13A oligonucleotide with spacers is designed such that 1) each segment of the UNC13A oligonucleotide has at most 7 linked nucleosides and 2) at least two, three, or four spacers are positioned adjacent to a guanine group. In some embodiments, a UNC13A oligonucleotide with spacers is designed such that 1) each segment of the UNC13A oligonucleotide has at most 7 linked nucleosides and 2) each of two spacers precede a guanine group.

In various embodiments, the inclusion of one or more spacers in the UNC13A oligonucleotide does not decrease the effectiveness of the UNC13A oligonucleotide with the spacers in restoring full length UNC13A protein or full length UNC13A mRNA in comparison to the effect of a corresponding UNC13A parent oligonucleotide. In various embodiments, the inclusion of one or more spacers in the UNC13A oligonucleotide increases the effectiveness of the UNC13A oligonucleotide with the spacers in restoring full length UNC13A protein or full length UNC13A mRNA in comparison to the effect of a corresponding UNC13A parent oligonucleotide. In various embodiments, the inclusion of one or more spacers in the UNC13A oligonucleotide does not decrease the effectiveness of the UNC13A oligonucleotide with the spacers in reducing quantity of UNC13A transcripts in comparison to the effect of a corresponding UNC13A parent oligonucleotide. In various embodiments, the inclusion of one or more spacers in the UNC13A oligonucleotide increases the effectiveness of the UNC13A oligonucleotide with the spacers in reducing quantity of UNC13A transcripts in comparison to the effect of a corresponding UNC13A parent oligonucleotide.

Tables 18A, 18B, 19, and 20 document example UNC13A oligonucleotides with one or more spacers and their relation to corresponding UNC13A parent oligonucleotides. Each UNC13A oligonucleotide is assigned a sequence name. As used hereafter, the nomenclature of the sequence name is expressed as “X_spA” (for a UNC13A AON with one spacer), “X_spA_spB” (for a UNC13A AON with two spacers), or “X_spA_spB_spC” (for a UNC13A AON with three spacers). Here, “X” refers to the length of the UNC13A AON, “A” refers to the position in the UNC13A AON where the first spacer is located, “B” refers to the position in the UNC13A AON where the second spacer is located, and if present, “C” refers to the position in the UNC13A AON where the third spacer is located.

In various embodiments, UNC13A oligonucleotides include one spacer. In various embodiments, the UNC13A oligonucleotides are oligonucleotide variants, such as any one of a 23mer, 21mer, or 19mer. In various embodiments, the inclusion of a spacer divides up the UNC13A oligonucleotide into two separate segments, where at least one of the segments is at most 11 linked nucleosides in length. In various embodiments, the inclusion of a spacer divides up the UNC13A oligonucleotide into two separate segments, where at least one of the segments is at most 10 linked nucleosides in length.

In various embodiments, the spacer is located between positions 10 and 15 of the oligonucleotide. In various embodiments, the spacer is located between positions 10 and 12 of the oligonucleotide. In particular embodiments, the spacer is located at position 10 of the oligonucleotide. In particular embodiments, the spacer is located at position 11 of the oligonucleotide. In particular embodiments, the spacer is located at position 12 of the oligonucleotide. In particular embodiments, the spacer is located at position 15 of the oligonucleotide. Example UNC13A AONs with one spacer are documented below in Table 18A.

TABLE 18A
Identification of UNC13A AONs with one spacer. Here, each UNC13A AON has 2
segments, where at least one of the segments has at most 11 linked nucleosides.

| | | | Sequence* (where X indicates a |
|---|---|---|---|
| | Relation to | | nucleoside of the KCNQ2 parent |
| | UNC13A | Sequence ID | oligonucleotide and S_{y} indicates |
| | oligonucleotide | Number | presence of a Spacer where y denotes |
| Sequence name | variant | (SEQ ID NO) | the position) (5′→3′) |
| UNC13A parent | N/A | 9596 | XXXXXXXXXXXXXXXXXXXXXXXXX |
| oligonucleotide | | | (25 mer) |
| UNC13A | Nucleoside at | 9597 | XXXXXXXXXXXXXXS₁₅XXXXXXXXXX |
| Oligonucleotide | position 15 of | | |
| (25 mer) | 25 mer is | | |
| with Spacer at | substituted with | | |
| position 15 | a spacer | | |
| (UNC13A AON | | | |
| 25_sp15) | | | |
| UNC13A | N/A | 9598 | XXXXXXXXXXXXXXXXXXXXXXX |
| oligonucleotide | | | (23 mer) |
| variant (23 mer) | | | |
| UNC13A | Nucleoside at | 9599 | XXXXXXXXXXXS₁₂XXXXXXXXXXX |
| Oligonucleotide | position 12 of | | |
| (23 mer) | 23 mer is | | |
| with Spacer at | substituted with | | |
| position 12 | a spacer | | |
| (UNC13A AON | | | |
| 23_sp12) | | | |
| UNC13A | N/A | 9600 | XXXXXXXXXXXXXXXXXXXXX |
| oligonucleotide | | | (21 mer) |
| variant (21 mer) | | | |
| UNC13A | Nucleoside at | 9601 | XXXXXXXXXXS₁₁XXXXXXXXXX |
| Oligonucleotide | position 11 of | | |
| (21 mer) | 21 mer is | | |
| with Spacer at | substituted with | | |
| position 11 | a spacer | | |
| (UNC13A AON | | | |
| 21_sp11) | | | |
| UNC13A | N/A | 9602 | XXXXXXXXXXXXXXXXXXX |
| oligonucleotide | | | (19 mer) |
| variant (19 mer) | | | |
| UNC13A | Nucleoside at | 9603 | XXXXXXXXXS₁₀XXXXXXXXX |
| Oligonucleotide | position 10 of | | |
| (19 mer) | 19 mer is | | |
| with Spacer at | substituted with | | |
| position 10 | a spacer | | |
| (UNC13A AON | | | |
| 19_sp10) | | | |

*At least one nucleoside linkage of the nucleobase sequence is selected from a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

In various embodiments, UNC13A oligonucleotides include two spacers. In various embodiments, the inclusion of a spacer divides up the UNC13A oligonucleotide into three separate segments, where at least one of the segments is at most 7 linked nucleosides in length. Example UNC13A AONs with two spacers are documented below in Table 18B.

TABLE 18B
Identification of UNC13A AONs with two spacers. Here, each UNC13A AON has 3
segments, where at least one of the segments has at most 7 linked nucleosides.

| | | | Sequence* (where X indicates a |
|---|---|---|---|
| | | | nucleoside of the UNC13A parent |
| | Relation to | Sequence ID | oligonucleotide and S_{y} indicates |
| | UNC13A parent | Number | presence of a Spacer where y denotes |
| Sequence name | oligonucleotide | (SEQ ID NO) | the position) (5′→3′) |
| UNC13A parent | N/A | 9604 | XXXXXXXXXXXXXXXXXXXXXXXXX |
| oligonucleotide | | | |
| UNC13A | Nucleosides at | 9605 | XXXXXXXXXXS₁₁XXXXXXXXXXS₂₂XXX |
| Oligonucleotide | positions 11 and | | |
| with Spacers at | 22 are each | | |
| positions 11 and 22 | substituted with | | |
| (UNC13A AON | a spacer | | |
| 25_sp11sp22) | | | |
| UNC13A | Nucleosides at | 9606 | XXXXXXS₇XXXXXXS₁₄XXXXXXXXXXX |
| Oligonucleotide | positions 7 and | | |
| with Spacers at | 14 are each | | |
| positions 7 and 14 | substituted with | | |
| (UNC13A AON | a spacer | | |
| 25_sp7sp14) | | | |
| UNC13A | Nucleosides at | 9607 | XXXXXXXS₈XXXXXXXXXXS₁₉XXXXXX |
| Oligonucleotide | positions 8 and | | |
| with Spacers at | 19 are | | |
| positions 8 and 19 | substituted with | | |
| (UNC13A AON | spacers | | |
| 25_sp8sp19) | | | |
| UNC13A | Nucleosides at | 9608 | XXXXXXXS₈XXXXXS₁₄XXXXXXXXXXX |
| Oligonucleotide | positions 8 and | | |
| with Spacers at | 14 are | | |
| positions 8 and 14 | substituted with | | |
| (UNC13A AON | spacers | | |
| 25_sp8sp14) | | | |
| UNC13A | Nucleosides at | 9609 | XXXXXXXXS₉XXXXS₁₄XXXXXXXXXXX |
| Oligonucleotide | positions 9 and | | |
| with Spacers at | 14 are | | |
| positions 9 and 14 | substituted with | | |
| (UNC13A AON | spacers | | |
| 25_sp9sp14) | | | |
| UNC13A | Nucleosides at | 9610 | XXXXXXXXXS₁₀XXXS₁₄XXXXXXXXXXX |
| Oligonucleotide | positions 10 and | | |
| with Spacers at | 14 are | | |
| positions 10 and 14 | substituted with | | |
| (UNC13A AON | spacers | | |
| 25_sp10sp14) | | | |
| UNC13A | Nucleosides at | 9611 | XXXXXXXXXXS₁₁XXS₁₄XXXXXXXXXXX |
| Oligonucleotide | positions 11 and | | |
| with Spacers at | 14 are | | |
| positions 11 and 14 | substituted with | | |
| (UNC13A AON | spacers | | |
| 25_sp11sp14) | | | |
| UNC13A | Nucleosides at | 9612 | XXXXXXXS₈XXXXXXS₁₅XXXXXXXXXX |
| Oligonucleotide | positions 8 and | | |
| with Spacers at | 15 are | | |
| positions 8 and 15 | substituted with | | |
| (UNC13A AON | spacers | | |
| 25_sp8sp15) | | | |
| UNC13A | Nucleosides at | 9613 | XXXXXXXXS₉XXXXXS₁₅XXXXXXXXXX |
| Oligonucleotide | positions 9 and | | |
| with Spacers at | 15 are | | |
| positions 9 and 15 | substituted with | | |
| (UNC13A AON | spacers | | |
| 25_sp9sp15) | | | |
| UNC13A | Nucleosides at | 9614 | XXXXXXXXXS₁₀XXXXS₁₅XXXXXXXXXX |
| Oligonucleotide | positions 10 and | | |
| with Spacers at | 15 are | | |
| positions 10 and 15 | substituted with | | |
| (UNC13A AON | spacers | | |
| 25_sp10sp15) | | | |
| UNC13A | Nucleosides at | 9615 | XXXXXXXXXXS₁₁XXXS₁₅XXXXXXXXXX |
| Oligonucleotide | positions 11 and | | |
| with Spacers at | 15 are | | |
| positions 11 and 15 | substituted with | | |
| (UNC13A AON | spacers | | |
| 25_sp11sp15) | | | |
| UNC13A | Nucleosides at | 9616 | XXXXXXXS₈XXXXXXXS₁₆XXXXXXXXX |
| Oligonucleotide | positions 8 and | | |
| with Spacers at | 16 are | | |
| positions 8 and 16 | substituted with | | |
| (UNC13A AON | spacers | | |
| 25_sp8sp16) | | | |
| UNC13A | Nucleosides at | 9617 | XXXXXXXXS₉XXXXXXS₁₆XXXXXXXXX |
| Oligonucleotide | positions 9 and | | |
| with Spacers at | 16 are | | |
| positions 9 and 16 | substituted with | | |
| (UNC13A AON | spacers | | |
| 25_sp9sp16) | | | |
| UNC13A | Nucleosides at | 9618 | XXXXXXXXXS₁₀XXXXXS₁₆XXXXXXXXX |
| Oligonucleotide | positions 10 and | | |
| with Spacers at | 16 are | | |
| positions 10 and 16 | substituted with | | |
| (UNC13A AON | spacers | | |
| 25_sp10sp16) | | | |
| UNC13A | Nucleosides at | 9619 | XXXXXXXXXXS₁₁XXXXS₁₆XXXXXXXXX |
| Oligonucleotide | positions 11 and | | |
| with Spacers at | 16 are | | |
| positions 11 and 16 | substituted with | | |
| (UNC13A AON | spacers | | |
| 25_sp11sp16) | | | |
| UNC13A | Nucleosides at | 9620 | XXXXXXXS₈XXXXXXXXS₁₇XXXXXXXX |
| Oligonucleotide | positions 8 and | | |
| with Spacers at | 17 are | | |
| positions 8 and 17 | substituted with | | |
| (UNC13A AON | spacers | | |
| 25_sp8sp17) | | | |
| UNC13A | Nucleosides at | 9621 | XXXXXXXXS₉XXXXXXXS₁₇XXXXXXXX |
| Oligonucleotide | positions 9 and | | |
| with Spacers at | 17 are | | |
| positions 9 and 17 | substituted with | | |
| (UNC13A AON | spacers | | |
| 25_sp9sp17) | | | |
| UNC13A | Nucleosides at | 9622 | XXXXXXXXXS₁₀XXXXXXS₁₇XXXXXXXX |
| Oligonucleotide | positions 10 and | | |
| with Spacers at | 17 are | | |
| positions 10 and 17 | substituted with | | |
| (UNC13A AON | spacers | | |
| 25_sp10sp17) | | | |
| UNC13A | Nucleosides at | 9623 | XXXXXXXXXXS₁₁XXXXXS₁₇XXXXXXXX |
| Oligonucleotide | positions 11 and | | |
| with Spacers at | 17 are | | |
| positions 11 and 17 | substituted with | | |
| (UNC13A AON | spacers | | |
| 25_sp11sp17) | | | |
| UNC13A | Nucleosides at | 9624 | XXXXXXXS₈XXXXXXXXXS₁₈XXXXXXX |
| Oligonucleotide | positions 8 and | | |
| with Spacers at | 18 are | | |
| positions 8 and 18 | substituted with | | |
| (UNC13A ATON | spacers | | |
| 25_sp8sp18) | | | |
| UNC13A | Nucleosides at | 9625 | XXXXXXXXS₉XXXXXXXXS₁₈XXXXXXX |
| Oligonucleotide | positions 9 and | | |
| with Spacers at | 18 are | | |
| positions 9 and 18 | substituted with | | |
| (UNC13A AON | spacers | | |
| 25_sp9sp18) | | | |
| UNC13A | Nucleosides at | 9626 | XXXXXXXXXS₁₀XXXXXXXS₁₈XXXXXXX |
| Oligonucleotide | positions 10 and | | |
| with Spacers at | 18 are | | |
| positions 10 and 18 | substituted with | | |
| (UNC13A AON | spacers | | |
| 25_sp10sp18) | | | |
| UNC13A | Nucleosides at | 9627 | XXXXXXXXXXS₁₁XXXXXXS₁₈XXXXXXX |
| Oligonucleotide | positions 11 and | | |
| with Spacers at | 18 are | | |
| positions 11 and 18 | substituted with | | |
| (UNC13A AON | spacers | | |
| 25_sp11sp18) | | | |
| UNC13A | Nucleosides at | 9628 | XXXXXXXXS₉XXXXXXXXXS₁₉XXXXXX |
| Oligonucleotide | positions 9 and | | |
| with Spacers at | 19 are | | |
| positions 9 and 19 | substituted with | | |
| (UNC13A AON | spacers | | |
| 25_sp9sp19) | | | |
| UNC13A | Nucleosides at | 9629 | XXXXXXXXXS₁₀XXXXXXXXS₁₉XXXXXX |
| Oligonucleotide | positions 10 and | | |
| with Spacers at | 19 are | | |
| positions 10 and 19 | substituted with | | |
| (UNC13A AON | spacers | | |
| 25_sp10sp19) | | | |
| UNC13A | Nucleosides at | 9630 | XXXXXXXXXXS₁₁XXXXXXXS₁₉XXXXXX |
| Oligonucleotide | positions 11 and | | |
| with Spacers at | 19 are | | |
| positions 11 and 19 | substituted with | | |
| (UNC13A AON | spacers | | |
| 25_sp11sp19) | | | |
| UNC13A | Nucleosides at | 9631 | XXXXXXXXS₉XXXXXXXXXXS₂₀XXXXX |
| Oligonucleotide | positions 9 and | | |
| with Spacers at | 20 are | | |
| positions 9 and 20 | substituted with | | |
| (UNC13A AON | spacers | | |
| 25_sp9sp20) | | | |
| UNC13A | Nucleosides at | 9632 | XXXXXXXXXS₁₀XXXXXXXXXS₂₀XXXXX |
| Oligonucleotide | positions 10 and | | |
| with Spacers at | 20 are | | |
| positions 10 and 20 | substituted with | | |
| (UNC13A AON | spacers | | |
| 25_sp10sp20) | | | |
| UNC13A | Nucleosides at | 9633 | XXXXXXXXXXS₁₁XXXXXXXXS₂₀XXXXX |
| Oligonucleotide | positions 11 and | | |
| with Spacers at | 20 are | | |
| positions 11 and 20 | substituted with | | |
| (UNC13A AON | spacers | | |
| 25_sp11sp20) | | | |
| UNC13A | Nucleosides at | 9634 | XXXXXXXXXS₁₀XXXXXXXXXXS₂₁XXXX |
| Oligonucleotide | positions 10 and | | |
| with Spacers at | 21 are | | |
| positions 10 and 21 | substituted with | | |
| (UNC13A AON | spacers | | |
| 25_sp10sp21) | | | |
| UNC13A | Nucleosides at | 9635 | XXXXXXXXXXS₁₁XXXXXXXXXS₂₁XXXX |
| Oligonucleotide | positions 11 and | | |
| with Spacers at | 21 are | | |
| positions 11 and 21 | substituted with | | |
| (UNC13A AON | spacers | | |
| 25_sp11sp21) | | | |
| UNC13A | Nucleosides at | 9636 | XXXS₄XXXXXXXXXXS₁₅XXXXXXXXXXX |
| Oligonucleotide | positions 4 and | | |
| with Spacers at | 15 are | | |
| positions 4 and 15 | substituted with | | |
| (UNC13A AON | spacers | | |
| 25_sp4sp15) | | | |
| UNC13A | Nucleosides at | 9637 | XXXXXXS₇XXXXXXXXXXXS₁₉XXXXXX |
| Oligonucleotide | positions 7 and | | |
| with Spacers at | 19 are | | |
| positions 7 and 19 | substituted with | | |
| (UNC13A AON | spacers | | |
| 25_sp7sp19) | | | |
| UNC13A | Nucleosides at | 9638 | XXXXXXS₇XXXXXXXXXXS₁₈XXXXXXX |
| Oligonucleotide | positions 7 and | | |
| with Spacers at | 18 are | | |
| positions 7 and 18 | substituted with | | |
| (UNC13A AON | spacers | | |
| 25_sp7sp18) | | | |
| UNC13A | Nucleosides at | 9639 | XXXXXXXXS₉XXXXXXXXXXXS₂₁XXXX |
| Oligonucleotide | positions 9 and | | |
| with Spacers at | 21 are | | |
| positions 9 and 21 | substituted with | | |
| (UNC13A AON | spacers | | |
| 25_sp9sp21) | | | |

*At least one nucleoside linkage of the nucleobase sequence is selected from a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

In various embodiments, UNC13A oligonucleotides include three spacers. The inclusion of three spacers divides up the UNC13A oligonucleotide into four separate segments. In various embodiments, the three spacers are located at different positions of the UNC13A oligonucleotide such that each of the segments of the UNC13A oligonucleotide are at most 7 linked nucleosides in length. Example UNC13A AONs with three spacers are documented below in Table 19.

TABLE 19
Identification of UNC13A AONs or AON variants with three spacers. Here, each
UNC13A AON has 4 segments, where each segment has at most 7 linked nucleosides.

| | | | Sequence* (where X indicates a |
|---|---|---|---|
| | Relation to | | nucleoside of the UNC13A parent |
| | UNC13A | Sequence ID | oligonucleotide and S_{y} indicates |
| | parent | Number | presence of a Spacer where y denotes |
| Sequence name | oligonucleotide | (SEQ ID NO) | the position) (5′ → 3′) |
| UNC13A | Nucleosides | 9640 | XXXXXXXS8XXXXXXXS16XXXXXXXS24X |
| Oligonucleotide | at positions 8 | | |
| with Spacers at | and 16 and 24 | | |
| positions 8 and 16 | are substituted | | |
| and 24 (UNC13A | with spacers | | |
| AON | | | |
| 25_sp8sp16sp24) | | | |
| UNC13A | Nucleosides | 9641 | XXXXXXXS8XXXXXXXS16XXXXXXS23XX |
| Oligonucleotide | at positions 8 | | |
| with Spacers at | and 16 and 23 | | |
| positions 8 and 16 | are substituted | | |
| and 23 (UNC13A | with spacers | | |
| AON | | | |
| 25_sp8sp16sp23) | | | |
| UNC13A | Nucleosides | 9642 | XS2XXXXXXXS10XXXXXXXS18XXXXXXX |
| Oligonucleotide | at positions 2 | | |
| with Spacers at | and 10 and 18 | | |
| positions 2 and 10 | are substituted | | |
| and 18 (UNC13A | with spacers | | |
| AON | | | |
| 25_sp8sp16sp23) | | | |
| UNC13A | Nucleosides | 9643 | XXS3XXXXXXS10XXXXXXXS18XXXXXXX |
| Oligonucleotide | at positions 3 | | |
| with Spacers at | and 10 and 18 | | |
| positions 3 and 10 | are substituted | | |
| and 18 (UNC13A | with spacers | | |
| AON | | | |
| 25_sp8sp16sp23) | | | |
| UNC13A | Nucleosides | 9644 | XXXS4XXXXXXXS12XXXXXXS19XXXXXX |
| Oligonucleotide | at positions 4 | | |
| with Spacers at | and 12 and 19 | | |
| positions 4 and 12 | | | |
| and 19 (UNC13A | are substituted | | |
| AON | with spacers | | |
| 25_sp4sp12sp19) | | | |
| UNC13A | Nucleosides | 9645 | XXXXXXXS8XXXXS13XXXXS18XXXXXXX |
| Oligonucleotide | at positions 8 | | |
| with Spacers at | and 13 and 18 | | |
| positions 8 and 13 | are substituted | | |
| and 18 (UNC13A | with spacers | | |
| AON | | | |
| 25_sp8sp13sp18) | | | |
| UNC13A | Nucleosides | 9646 | XXXXS5XXXXXXXS13XXXXXXXS21XXXX |
| Oligonucleotide | at positions 5 | | |
| with Spacers at | and 13 and 21 | | |
| positions 5 and 13 | are substituted | | |
| and 21 (UNC13A | with spacers | | |
| AON | | | |
| 25_sp5sp13sp21) | | | |
| UNC13A | Nucleosides | 9647 | XXXXXXS7XXXXXS13XXXXXS19XXXXXX |
| Oligonucleotide | at positions 7 | | |
| with Spacers at | and 13 and 19 | | |
| positions 7 and 13 | are substituted | | |
| and 19 (UNC13A | with spacers | | |
| AON | | | |
| 25_sp7sp13sp19) | | | |
| UNC13A | Nucleosides | 9648 | XXXXXS8XXXXXXS13XXXXXXS20XXXXX |
| Oligonucleotide | at positions 6 | | |
| with Spacers at | and 13 and 20 | | |
| positions 6 and 13 | are substituted | | |
| and 20 (UNC13A | with spacers | | |
| AON | | | |
| 25_sp6sp13sp20) | | | |
| UNC13A | Nucleosides | 9649 | XXXXXXXS8XXS11XXXXXXXS19XXXXXX |
| Oligonucleotide | at positions 8 | | |
| with Spacers at | and 11 and 19 | | |
| positions 8 and 11 | are substituted | | |
| and 19 (UNC13A | with spacers | | |
| AON | | | |
| 25_sp8sp11sp19) | | | |
| UNC13A | Nucleosides | 9650 | XXXXXXXS8XXS11XXXXS16XXXXXXX |
| Oligonucleotide | at positions 8 | | |
| with Spacers at | and 11 and 16 | | |
| positions 8 and 11 | are substituted | | |
| and 16 (UNC13A | with spacers | | |
| AON | | | |
| 23_sp8sp11sp16) | | | |
| UNC13A | Nucleosides | 9651 | XXXXXXS7XXXXXXS14XXXXXXXS22XXX |
| Oligonucleotide | at positions 7 | | |
| with Spacers at | and 14 and 22 | | |
| positions 7 and 14 | | | |
| and 22 (UNC13A | are substituted | | |
| AON | with spacers | | |
| 23_sp7sp14sp22) | | | |

*At least one nucleoside linkage of the nucleobase sequence is selected from a phosphorothioate
linkage, an alkyl phosphate linkage, a phosphorodithioate linkage, a phosphotriester linkage, an
alkylphosphonate linkage, a 3-methoxypropyl phosphonate linkage, a methylphosphonate linkage, an
aminoalkylphosphotriester linkage, an alkylene phosphonate linkage, a phosphinate linkage, a
phosphoramidate linkage, a phosphoramidothioate linkage, a phosphorodiamidate (e.g., comprising
a phosphorodiamidate morpholino (PMO), 3′ amino ribose, or 5′ amino ribose) linkage, an
aminoalkylphosphoramidate linkage, a thiophosphoramidate linkage, a thionoalkylphosphonate
linkage, a thionoalkylphosphotriester linkage, a thiophosphate linkage, a selenophosphate
linkage, and a boranophosphate linkage.

In various embodiments, UNC13A AONs with one or more spacers are reduced in length in comparison to the UNC13A AONs described above in Tables 18B and 19. For example, such UNC13A AONs may be UNC13A oligonucleotide variants with one or more spacers. In various embodiments, the UNC13A oligonucleotide variants with one or more spacers are 23mers, 21mers, or 19mers. In various embodiments, UNC13A oligonucleotide variants include two spacers such that the UNC13A oligonucleotide variant includes three segments that are divided up by the two spacers. In various embodiments, at least one of the three segments has at most 7 linked nucleosides. In various embodiments, each of the three segments has at most 7 linked nucleosides. Example UNC13A oligonucleotide variants with one or more spacers are shown below in Table 20.

TABLE 20
Example UNC13A AON variants with two spacers. Here, each UNC13A AON variant
has 3 segments, where at least one segment has at most 7 linked nucleosides.

| | | | Sequence* (where X indicates a |
|---|---|---|---|
| | Relation to | | nucleoside of the UNC13A |
| | UNC13A | Sequence ID | oligonucleotide variant and S_{y} indicates |
| | oligonucleotide | Number | presence of a Spacer where y denotes the |
| Sequence name | variant | (SEQ ID NO) | position) (5′ → 3′) |
| UNC13A | N/A | 9652 | XXXXXXXXXXXXXXXXXXXXXXX (23mer) |
| oligonucleotide | | | |
| variant (23mer) | | | |
| UNC13A | Nucleosides at | 9653 | XXXXXXXS8XXXXXXXS16XXXXXXX |
| Variant | positions 8 and | | |
| Oligonucleotide | 16 are | | |
| (23mer) with | substituted with | | |
| Spacers at | spacers | | |
| positions 8 and | | | |
| 16 (UNC13A | | | |
| AON variant | | | |
| 23_sp8sp16) | | | |
| UNC13A | N/A | 9654 | XXXXXXXXXXXXXXXXXXXXX |
| oligonucleotide | | | (21mer) |
| variant (21mer) | | | |
| UNC13A | Nucleosides at | 9655 | XXXXS5XXXXXXS12XXXXXXXXX |
| Variant | positions 5 and | | |
| Oligonucleotide | 12 are | | |
| (21mer) with | substituted with | | |
| Spacers at | spacers | | |
| positions 5 and | | | |
| 12 (UNC13A | | | |
| AON variant | | | |
| 21_sp5sp12) | | | |
| UNC13A | Nucleosides at | 9656 | XXXXXXXS8XXXXXXXS16XXXXX |
| Variant | positions 8 and | | |
| Oligonucleotide | 16 are | | |
| (21mer) with | substituted with | | |
| Spacers at | spacers | | |
| positions 8 and | | | |
| 16 (UNC13A | | | |
| AON variant | | | |
| 21_sp8sp16) | | | |
| UNC13A | Nucleosides at | 9657 | XXXXXS6XXXXXXXS14XXXXXXX |
| Variant | positions 6 and | | |
| Oligonucleotide | 14 are | | |
| (21mer) with | substituted with | | |
| Spacers at | spacers | | |
| positions 6 and | | | |
| 14 (UNC13A | | | |
| AON variant | | | |
| 21_sp6sp14) | | | |
| UNC13A | Nucleosides at | 9658 | XXXXXXXS8XXXXXS14XXXXXXX |
| Variant | positions 8 and | | |
| Oligonucleotide | 14 are | | |
| (21mer) with | substituted with | | |
| Spacers at | spacers | | |
| positions 8 and | | | |
| 14 (UNC13A | | | |
| AON variant | | | |
| 21_sp8sp14) | | | |
| UNC13A | Nucleosides at | 9659 | XXXXXS8XXXXXXXXXXXXXS20X |
| Variant | positions 6 and | | |
| Oligonucleotide | 20 are | | |
| (21mer) with | substituted with | | |
| Spacers at | spacers | | |
| positions 8 and | | | |
| 14 (UNC13A | | | |
| AON variant | | | |
| 21 sp8sp14) | | | |
| UNC13A | N/A | 9660 | XXXXXXXXXXXXXXXXXXX |
| oligonucleotide | | | (19mer) |
| variant (19mer) | | | |
| UNC13A | Nucleosides at | 9661 | XXXXS5XXXXXXS12XXXXXXX |
| Variant | positions 5 and | | |
| Oligonucleotide | 12 are | | |
| (19mer) with | substituted with | | |
| Spacers at | spacers | | |
| positions 5 and | | | |
| 12 (UNC13A | | | |
| AON variant | | | |
| 19_sp5sp12) | | | |
| UNC13A | Nucleosides at | 9662 | XXXXXXXS8XXXXXXS15XXXX |
| Variant | positions 8 and | | |
| Oligonucleotide | 15 are | | |
| (19mer) with | substituted with | | |
| Spacers at | spacers | | |
| positions 8 and | | | |
| 15 (UNC13A | | | |
| AON variant | | | |
| 19_sp8sp15) | | | |

*At least one nucleoside linkage of the nucleobase sequence is selected from a phosphorothioate
linkage, an alkyl phosphate linkage, a phosphorodithioate linkage, a phosphotriester linkage, an
alkylphosphonate linkage, a 3-methoxypropyl phosphonate linkage, a methylphosphonate
linkage, an aminoalkylphosphotriester linkage, an alkylene phosphonate linkage, a phosphinate
linkage, a phosphoramidate linkage, a phosphoramidothioate linkage, a phosphorodiamidate
(e.g., comprising a phosphorodiamidate morpholino (PMO), 3′ amino ribose, or 5′ amino ribose)
linkage, an aminoalkylphosphoramidate linkage, a thiophosphoramidate linkage, a
thionoalkylphosphonate linkage, a thionoalkylphosphotriester linkage, a thiophosphate linkage, a
selenophosphate linkage, and a boranophosphate linkage.

In some embodiments, an antisense oligonucleotide disclosed herein (e.g., UNC13A parent oligonucleotides and/or UNC13A oligonucleotide variants) comprise one or more spacers as well as one or more locked nucleic acids (LNAs). In some embodiments, an antisense oligonucleotide disclosed herein (e.g., UNC13A parent oligonucleotides and/or UNC13A oligonucleotide variants) comprises two spacers and two LNAs. In some embodiments, an antisense oligonucleotide disclosed herein (e.g., UNC13A parent oligonucleotides and/or UNC13A oligonucleotide variants) comprises two spacers and three LNAs.

In various embodiments, a spacer and a LNA are located adjacent to one another in an antisense oligonucleotide. For example, if counting from 5′ to 3′, a LNA can be located at a 7^{th} position of the antisense oligonucleotide and a spacer can be located at a 8^{th} position of the antisense oligonucleotide. As another example, if counting from 5′ to 3′, a LNA can be located at a 9^{th} position of the antisense oligonucleotide and a spacer can be located at a 8^{th} position of the antisense oligonucleotide. As another example, if counting from 5′ to 3′, a LNA can be located at a 15^{th} position of the antisense oligonucleotide and a spacer can be located at a 16^{th} position of the antisense oligonucleotide. As another example, if counting from 5′ to 3′, a LNA can be located at a 17^{th} position of the antisense oligonucleotide and a spacer can be located at a 16^{th} position of the antisense oligonucleotide.

In particular embodiments, a first spacer is located adjacent to a first LNA and a second spacer is located adjacent to a second LNA in an antisense oligonucleotide. For example, if counting from 5′ to 3′, a first LNA can be located at a 7^{th} position of the antisense oligonucleotide, a first spacer can be located at a 8^{th} position of the antisense oligonucleotide, a second LNA can be located at a 15^{th} position of the antisense oligonucleotide, and a second spacer can be located at a 16^{th} position of the antisense oligonucleotide. As another example, if counting from 5′ to 3′, a first LNA can be located at a 7^{th} position of the antisense oligonucleotide, a first spacer can be located at a 8^{th} position of the antisense oligonucleotide, a second LNA can be located at a 17^{th} position of the antisense oligonucleotide, and a second spacer can be located at a 16^{th} position of the antisense oligonucleotide. As another example, if counting from 5′ to 3′, a first LNA can be located at a 9^{th} position of the antisense oligonucleotide, a first spacer can be located at a 8^{th} position of the antisense oligonucleotide, a second LNA can be located at a 17m position of the antisense oligonucleotide, and a second spacer can be located at a 16^{th} position of the antisense oligonucleotide.

In various embodiments, one or more spacers and one or more LNAs are not located adjacent to one another in an antisense oligonucleotide. For example, if counting from 5′ to 3′, a LNA can be located at a 4^{th} position of the antisense oligonucleotide and a spacer can be located at a 8^{th} position of the antisense oligonucleotide. As example, if counting from 5′ to 3′, a LNA can be located at a 20^{th} position of the antisense oligonucleotide and a spacer can be located at a 16^{th} position of the antisense oligonucleotide. In particular embodiments, if counting from 5′ to 3′, a first LNA can be located at a 4^{th} position of the antisense oligonucleotide, a first spacer can be located at a 8^{th} position of the antisense oligonucleotide, a second LNA can be located at a 20^{th} position of the antisense oligonucleotide, and a second spacer can be located at a 16^{th} position of the antisense oligonucleotide. In particular embodiments, if counting from 5′ to 3′, a first LNA can be located at a 4^{th} position of the antisense oligonucleotide, a first spacer can be located at a 8^{th} position of the antisense oligonucleotide, a second LNA can be located at a 12^{th} position of the antisense oligonucleotide, a second spacer can be located at a 16^{th} position of the antisense oligonucleotide, and a third LNA can be located at a 20^{th} position of the antisense oligonucleotide.

### SMN2

As described further below, a SMN2 oligonucleotide with one or more spacers is described in reference to a corresponding SMN2 parent oligonucleotide. In various embodiments, a SMN2 oligonucleotide with a spacer differs from a SMN2 parent oligonucleotide in that the spacer replaces a nucleoside in the SMN2 parent oligonucleotide. As used hereafter, the “position” of the SMN2 oligonucleotide refers to a particular location as counted from the 5′ end of the SMN2 oligonucleotide. In various embodiments, the spacer replaces a nucleoside at any one of positions 1, 2, 3, 4, 5, 6, 7, 8, 9, 10, 11, 12, 13, 14, 15, 16, 17, 18, 19, 20, 21, 22, 23, 24, or 25 of the SMN2 parent oligonucleotide. In particular embodiments, a spacer replaces a nucleoside at one of positions 7, 8, 11, 14, 16, 19, or 22 of the SMN2 parent oligonucleotide.

In various embodiments, a SMN2 oligonucleotide includes one spacer that replaces a nucleoside in the SMN2 parent oligonucleotide (e.g., one spacer replaces one nucleoside of the SMN2 parent oligonucleotide). In particular embodiments, the spacer replaces a nucleoside between positions 9 and 15 of the SMN2 parent oligonucleotide. In particular embodiments, the spacer replaces a nucleoside between positions 9 and 12 of the SMN2 parent oligonucleotide. In particular embodiments, the spacer replaces a nucleoside at position 10 of the SMN2 parent oligonucleotide. In particular embodiments, the spacer replaces a nucleoside at position 11 of the SMN2 parent oligonucleotide. In particular embodiments, the spacer replaces a nucleoside at position 12 of the SMN2 parent oligonucleotide. In particular embodiments, the spacer replaces a nucleoside between positions 12 and 16 of the SMN2 parent oligonucleotide. In particular embodiments, the spacer replaces a nucleoside at position 15 of the SMN2 parent oligonucleotide.

In various embodiments, a SMN2 oligonucleotide including one spacer has 2 segments, where at least one of the 2 segments has at most 11 linked nucleosides. For example, the SMN2 oligonucleotide may be 23 oligonucleotide units in length, and the spacer can be located at position 12. Therefore, the SMN2 oligonucleotide has 2 segments divided by the spacer, where both of the 2 segments are 11 nucleobases in length. In various embodiments, a SMN2 oligonucleotide including one spacer has 2 segments, where at least one of the 2 segments has at most 10 linked nucleosides. For example, the SMN2 oligonucleotide may be 21 oligonucleotide units in length, and the spacer can be located at position 11. Therefore, the SMN2 oligonucleotide has 2 segments divided by the spacer, where both of the 2 segments are 10 nucleobases in length. As another example, the SMN2 oligonucleotide may be 25 oligonucleotide units in length, and the spacer can be located at position 15. Therefore, the SMN2 oligonucleotide has 2 segments divided by the spacer, where one of the 2 segments is 14 nucleobases in length and the second of the 2 segments is 10 nucleobases in length.

In various embodiments, a SMN2 oligonucleotide includes two spacers that each replace a nucleoside in the SMN2 parent oligonucleotide (e.g., two spacers replace two separate nucleosides of the SMN2 parent oligonucleotide). In various embodiments, a first spacer and a second spacer are separated by at least 5 nucleobases, at least 6 nucleobases, at least 7 nucleobases, at least 8 nucleobases, at least 9 nucleobases, or at least 10 nucleobases in the oligonucleotide. In particular embodiments, a first spacer and a second spacer are separated by at least 5 nucleobases, at least 6 nucleobases, or at least 7 nucleobases. In particular embodiments, the first spacer and the second spacer are not adjacent to one another in the oligonucleotide.

In particular embodiments, the first spacer replaces a nucleoside between positions 7 and 11 of the SMN2 parent oligonucleotide. In various embodiments, the first spacer replaces a nucleoside between positions 8 and 11, positions 9 and 11, positions 10 and 11, positions 7 and 10, positions 7 and 9, positions 7 and 8, positions 8 and 10, positions 8 and 9, or positions 9 and 10 of the SMN2 parent oligonucleotide. In particular embodiments, the second spacer replaces a nucleoside between positions 14 and 22 of the SMN2 parent oligonucleotide. In various embodiments, the second spacer replaces a nucleoside between positions 15 and 22, positions 16 and 22, positions 17 and 22, position 18 and 22, position 19 and 22, positions 20 and 22, positions 21 and 22, positions 15 and 21, position 16 and 21, positions 17 and 21, positions 18 and 21, positions 19 and 21, positions 20 and 21, positions 15 and 20, positions 16 and 20, positions 17 and 20, positions 18 and 20, positions 19 and 20, positions 15 and 19, positions 16 and 19, positions 17 and 19, positions 18 and 19, positions 15 and 18, position 16 and 18, position 17 and 18, positions 15 and 17, positions 16 and 17, or positions 15 and 16 of the SMN2 parent oligonucleotide.

In preferred embodiments, the first spacer replaces a nucleoside at position 7 of the SMN2 parent oligonucleotide and the second spacer replaces a nucleoside at position 14 of the SMN2 parent oligonucleotide. In preferred embodiments, the first spacer replaces a nucleoside at position 7 of the SMN2 parent oligonucleotide and the second spacer replaces a nucleoside at position 19 of the SMN2 parent oligonucleotide. In preferred embodiments, the first spacer replaces a nucleoside at position 8 of the SMN2 parent oligonucleotide and the second spacer replaces a nucleoside at position 16 of the SMN2 parent oligonucleotide. In preferred embodiments, the first spacer replaces a nucleoside at position 11 of the SMN2 parent oligonucleotide and the second spacer replaces a nucleoside at position 22 of the SMN2 parent oligonucleotide. In preferred embodiments, the first spacer replaces a nucleoside at position 9 of the SMN2 parent oligonucleotide and the second spacer replaces a nucleoside at position 19 of the SMN2 parent oligonucleotide.

In various embodiments, a SMN2 oligonucleotide includes three spacers that each replace a nucleoside in the SMN2 parent oligonucleotide (e.g., three spacers replace three separate nucleosides of the SMN2 parent oligonucleotide). In particular embodiments, the first spacer replaces a nucleoside between positions 7 and 11 of the SMN2 parent oligonucleotide. In particular embodiments, the second spacer replaces a nucleoside between positions 14 and 22 of the SMN2 parent oligonucleotide. In particular embodiments, the third spacer replaces a nucleoside between positions 21 and 24 of the SMN2 parent oligonucleotide. In some embodiments, the first spacer replaces a nucleoside between positions 2 and 5 of the SMN2 parent oligonucleotide. In particular embodiments, the second spacer replaces a nucleoside between positions 8 and 12 of the SMN2 parent oligonucleotide. In particular embodiments, the third spacer replaces a nucleoside between positions 18 and 22 of the SMN2 parent oligonucleotide.

In various embodiments, the three spacers in a SMN2 oligonucleotide are positioned such that each of the four segments of the SMN2 oligonucleotide are at most 7 linked nucleosides in length. For example, a SMN2 oligonucleotide may have a first segment with 7 linked nucleosides connected to a first spacer, then a second segment with 7 linked nucleosides connected on one end to the first spacer and connected on another end to a second spacer, then a third segment with 6 linked nucleosides connected on one end to the second spacer and connected on another end to a third spacer, then a fourth segment with 6 linked nucleosides connected to the third spacer.

In various embodiments, the one or more spacers are positioned in the oligonucleotide to replace one or more adenosine or thymine nucleosides (as opposed to guanine or cytosine nucleosides). For example, the one or more spacers can replace one, two, three, four, five, six, seven, eight, or nine adenosine or thymine nucleosides in the oligonucleotide. In various embodiments, the one or more spacers are positioned in the oligonucleotide to replace one or more guanine or cytosine nucleosides (as opposed to adenosine or thymine nucleosides). For example, the one or more spacers can replace one, two, three, four, five, six, seven, eight, or nine guanine or cytosine nucleosides in the oligonucleotide. In various embodiments, the spacers are positioned in the oligonucleotide to replace an equal number of adenosine/thymine nucleosides and guanine/cytosine nucleosides. For example, a first spacer in the oligonucleotide may replace an adenosine/thymine nucleoside and a second spacer in the oligonucleotide may replace a guanine/cytosine nucleoside.

In various embodiments, the one or more spacers are positioned in the oligonucleotide to control the sequence content in the oligonucleotide. For example, the one or more spacers are positioned such that at least one of the spacers is located adjacent to a guanine group. In various embodiments, an oligonucleotide with spacers can include one spacer adjacent to a guanine group, two spacers adjacent to guanine groups, three spacers adjacent to guanine groups, four spacers adjacent to guanine groups, or five spacers adjacent to guanine groups. In one embodiment, if counting from the 5′ end of the oligonucleotide, a spacer immediately precedes a guanine group in the sequence. Thus, in various embodiments, an oligonucleotide with spacers can include one spacer that immediately precedes a guanine group, two spacers that each immediately precede a guanine group, three spacers that each immediately precede a guanine group, four spacers that each immediately precede a guanine group, or five spacers that each immediately precede a guanine group. In one embodiment, if counting from the 5′ end of the oligonucleotide, a guanine group is immediately succeeded by a spacer. Thus, in various embodiments, an oligonucleotide with spacers can include one spacer that immediately succeeds a guanine group, two spacers that each immediately succeed a guanine group, three spacers that each immediately succeed a guanine group, four spacers that each immediately succeed a guanine group, or five spacers that each immediately succeed a guanine group. In various embodiments, the spacers in the oligonucleotide can be positioned to maximize the number of spacers adjacent to guanine groups.

In various embodiments, the one or more spacers are positioned in the oligonucleotide to replace one or more adenosine or thymine nucleosides such that the one or more spacers are located adjacent guanine groups. For example, two spacers can replace adenosine or thymine nucleosides in the oligonucleotide, each of the two spacers being located adjacent to a guanine group.

In various embodiments, the SMN2 oligonucleotide with one or more spacers has a particular GC content. As used herein, GC content (or guanine-cytosine content) is the percentage of nitrogenous bases in the oligonucleotide that are either guanine (G) or cytosine (C). In various embodiments, the SMN2 oligonucleotide with one or more spacers has at least 10% GC content, at least 20% GC content, at least 25% GC content, at least 30% GC content, at least 35% GC content, at least 40% GC content, at least 45% GC content, at least 50% GC content, at least 55% GC content, at least 60% GC content, at least 65% GC content, at least 75% GC content, at least 80% GC content, at least 85% GC content, at least 90% GC content, or at least 95% GC content. In particular embodiments, the SMN2 oligonucleotide with one or more spacers has at least 30% GC content. In particular embodiments, the SMN2 oligonucleotide with one or more spacers has at least 40% GC content. In various embodiments, the one or more spacers are positioned in the SMN2 oligonucleotide to maximize GC content. For example, instead of selecting a guanine or cytosine for replacement by a spacer in the SMN2 oligonucleotide, a thymine or adenine can be selected for replacement by a spacer.

In various embodiments, a SMN2 oligonucleotide with spacers is designed such that 1) each segment of the SMN2 oligonucleotide has at most 7 linked nucleosides and 2) at least two, three, or four spacers are positioned adjacent to a guanine group. In some embodiments, a SMN2 oligonucleotide with spacers is designed such that 1) each segment of the SMN2 oligonucleotide has at most 7 linked nucleosides and 2) each of two spacers precede a guanine group.

In various embodiments, the inclusion of one or more spacers in the SMN2 oligonucleotide does not decrease the effectiveness of the SMN2 oligonucleotide with the spacers in restoring full length SMN2 protein or full length SMN2 mRNA in comparison to the effect of a corresponding SMN2 parent oligonucleotide. In various embodiments, the inclusion of one or more spacers in the SMN2 oligonucleotide increases the effectiveness of the SMN2 oligonucleotide with the spacers in restoring full length SMN2 protein or full length SMN2 mRNA in comparison to the effect of a corresponding SMN2 parent oligonucleotide. In various embodiments, the inclusion of one or more spacers in the SMN2 oligonucleotide does not decrease the effectiveness of the SMN2 oligonucleotide with the spacers in reducing quantity of SMN2 transcripts in comparison to the effect of a corresponding SMN2 parent oligonucleotide. In various embodiments, the inclusion of one or more spacers in the SMN2 oligonucleotide increases the effectiveness of the SMN2 oligonucleotide with the spacers in reducing quantity of SMN2 transcripts in comparison to the effect of a corresponding SMN2 parent oligonucleotide.

Tables 21A, 21B, 22, and 23 document example SMN2 oligonucleotides with one or more spacers and their relation to corresponding SMN2 parent oligonucleotides. Each SMN2 oligonucleotide is assigned a sequence name. As used hereafter, the nomenclature of the sequence name is expressed as “X_spA” (for a SMN2 AON with one spacer), “X_spA_spB” (for a SMN2 AON with two spacers), or “X_spA_spB_spC” (for a SMN2 AON with three spacers). Here, “X” refers to the length of the SMN2 AON, “A” refers to the position in the SMN2 AON where the first spacer is located, “B” refers to the position in the SMN2 AON where the second spacer is located, and if present, “C” refers to the position in the SMN2 AON where the third spacer is located.

In various embodiments, SMN2 oligonucleotides include one spacer. In various embodiments, the SMN2 oligonucleotides are oligonucleotide variants, such as any one of a 23mer, 21mer, or 19mer. In various embodiments, the inclusion of a spacer divides up the SMN2 oligonucleotide into two separate segments, where at least one of the segments is at most 11 linked nucleosides in length. In various embodiments, the inclusion of a spacer divides up the SMN2 oligonucleotide into two separate segments, where at least one of the segments is at most 10 linked nucleosides in length.

In various embodiments, the spacer is located between positions 10 and 15 of the oligonucleotide. In various embodiments, the spacer is located between positions 10 and 12 of the oligonucleotide. In particular embodiments, the spacer is located at position 10 of the oligonucleotide. In particular embodiments, the spacer is located at position 11 of the oligonucleotide. In particular embodiments, the spacer is located at position 12 of the oligonucleotide. In particular embodiments, the spacer is located at position 15 of the oligonucleotide. Example SMN2 AONs with one spacer are documented below in Table 21A.

TABLE 21A
Identification of SMN2 AONs with one spacer. Here, each SMN2 AON has 2
segments, where at least one of the segments has at most 11 linked nucleosides.

| | | | Sequence* (where X indicates a |
|---|---|---|---|
| | Relation to | Sequence | nucleoside of the SMN2 parent |
| | SMN2 | ID | oligonucleotide and S_{y} indicates |
| | oligonucleotide | Number | presence of a Spacer where y |
| Sequence name | variant | (SEQ ID NO) | denotes the position) (5′ → 3′) |
| SMN2 parent | N/A | 10574 | XXXXXXXXXXXXXXXXXXXXXXXXX |
| oligonucleotide | | | (25mer) |
| SMN2 Oligonucleotide | Nucleoside at | 10575 | XXXXXXXXXXXXXXS15XXXXXXXXXX |
| (25mer) with Spacer at | position 15 of | | |
| position 15 | 25mer is | | |
| (SMN2 AON 25_sp15) | substituted with | | |
| | a spacer | | |
| SMN2 oligonucleotide | N/A | 10576 | XXXXXXXXXXXXXXXXXXXXXXX |
| variant (23mer) | | | (23mer) |
| SMN2 Oligonucleotide | Nucleoside at | 10577 | XXXXXXXXXXXS12XXXXXXXXXXX |
| (23mer) with Spacer at | position 12 of | | |
| position 12 | 23mer is | | |
| (SMN2 AON 23_sp12) | substituted with | | |
| | a spacer | | |
| SMN2 oligonucleotide | N/A | 10578 | XXXXXXXXXXXXXXXXXXXXX |
| variant (21mer) | | | (21mer) |
| SMN2 Oligonucleotide | Nucleoside at | 10579 | XXXXXXXXXXS11XXXXXXXXXX |
| (21mer) with Spacer at | position 11 of | | |
| position 11 | 21mer is | | |
| (SMN2 AON 21_sp11) | substituted with | | |
| | a spacer | | |
| SMN2 oligonucleotide | N/A | 10580 | XXXXXXXXXXXXXXXXXXX |
| variant (19mer) | | | (19mer) |
| SMN2 Oligonucleotide | Nucleoside at | 10581 | XXXXXXXXXS10XXXXXXXXX |
| (19mer) with Spacer at | position 10 of | | |
| position 10 | 19mer is | | |
| (SMN2 AON 19_sp10) | substituted with | | |
| | a spacer | | |

* At least one nucleoside linkage of the nucleobase sequence is selected from a phosphorothioate
linkage, an alkyl phosphate linkage, a phosphorodithioate linkage, a phosphotriester linkage, an
alkylphosphonate linkage, a 3-methoxypropyl phosphonate linkage, a methylphosphonate
linkage, an aminoalkylphosphotriester linkage, an alkylene phosphonate linkage, a phosphinate
linkage, a phosphoramidate linkage, a phosphoramidothioate linkage, a phosphorodiamidate
(e.g., comprising a phosphorodiamidate morpholino (PMO), 3′ amino ribose, or 5′ amino ribose)
linkage, an aminoalkylphosphoramidate linkage, a thiophosphoramidate linkage, a
thionoalkylphosphonate linkage, a thionoalkylphosphotriester linkage, a thiophosphate linkage, a
selenophosphate linkage, and a boranophosphate linkage.

In various embodiments, SMN2 oligonucleotides include two spacers. In various embodiments, the inclusion of a spacer divides up the SMN2 oligonucleotide into three separate segments, where at least one of the segments is at most 7 linked nucleosides in length. Example SMN2 AONs with two spacer are documented below in Table 22B.

TABLE 22B
Identification of SMN2 AONs with two spacers. Here, each SMN2 AON has 3
segments, where at least one of the segments has at most 7 linked nucleosides.

| | | | Sequence* (where X indicates a |
|---|---|---|---|
| | | | nucleoside of the SMN2 parent |
| | Relation to | Sequence ID | oligonucleotide and S_{y} indicates |
| | SMN2 parent | Number | presence of a Spacer where y denotes |
| Sequence name | oligonucleotide | (SEQ ID NO) | the position) (5′ → 3′) |
| SMN2 parent | N/A | 10582 | XXXXXXXXXXXXXXXXXXXXXXXXX |
| oligonucleotide | | | |
| SMN2 | Nucleosides at | 10583 | XXXXXXXXXXS11XXXXXXXXXXS22XXX |
| Oligonucleotide | positions 11 and | | |
| with Spacers at | 22 are each | | |
| positions 11 and 22 | substituted with | | |
| (SMN2 AON | a spacer | | |
| 25_sp11sp22) | | | |
| SMN2 | Nucleosides at | 10584 | XXXXXXS7XXXXXXS14XXXXXXXXXXX |
| Oligonucleotide | positions 7 and | | |
| with Spacers at | 14 are each | | |
| positions 7 and 14 | substituted with | | |
| (SMN2 AON | a spacer | | |
| 25_sp7sp14) | | | |
| SMN2 | Nucleosides at | 10585 | XXXXXXXS8XXXXXXXXXXS19XXXXXX |
| Oligonucleotide | positions 8 and | | |
| with Spacers at | 19 are | | |
| positions 8 and 19 | substituted with | | |
| (SMN2 AON | spacers | | |
| 25_sp8sp19) | | | |
| SMN2 | Nucleosides at | 10586 | XXXXXXXS8XXXXXS14XXXXXXXXXXX |
| Oligonucleotide | positions 8 and | | |
| with Spacers at | 14 are | | |
| positions 8 and 14 | substituted with | | |
| (SMN2 AON | spacers | | |
| 25_sp8sp14) | | | |
| SMN2 | Nucleosides at | 10587 | XXXXXXXXS9XXXXS14XXXXXXXXXXX |
| Oligonucleotide | positions 9 and | | |
| with Spacers at | 14 are | | |
| positions 9 and 14 | substituted with | | |
| (SMN2 AON | spacers | | |
| 25_sp9sp14) | | | |
| SMN2 | Nucleosides at | 10588 | XXXXXXXXXS10XXXS14XXXXXXXXXXX |
| Oligonucleotide | positions 10 and | | |
| with Spacers at | 14 are | | |
| positions 10 and 14 | substituted with | | |
| (SMN2 AON | spacers | | |
| 25_sp10sp14) | | | |
| SMN2 | Nucleosides at | 10589 | XXXXXXXXXXS11XXS14XXXXXXXXXXX |
| Oligonucleotide | positions 11 and | | |
| with Spacers at | 14 are | | |
| positions 11 and 14 | substituted with | | |
| (SMN2 AON | spacers | | |
| 25_sp11sp14) | | | |
| SMN2 | Nucleosides at | 10590 | XXXXXXXS8XXXXXXS15XXXXXXXXXX |
| Oligonucleotide | positions 8 and | | |
| with Spacers at | 15 are | | |
| positions 8 and 15 | substituted with | | |
| (SMN2 AON | spacers | | |
| 25_sp8sp15) | | | |
| SMN2 | Nucleosides at | 10591 | XXXXXXXXS9XXXXXS15XXXXXXXXXX |
| Oligonucleotide | positions 9 and | | |
| with Spacers at | 15 are | | |
| positions 9 and 15 | substituted with | | |
| (SMN2 AON | spacers | | |
| 25_sp9sp15) | | | |
| SMN2 | Nucleosides at | 10592 | XXXXXXXXXS10XXXXS15XXXXXXXXXX |
| Oligonucleotide | positions 10 and | | |
| with Spacers at | 15 are | | |
| positions 10 and 15 | substituted with | | |
| (SMN2 AON | spacers | | |
| 25_sp10sp15) | | | |
| SMN2 | Nucleosides at | 10593 | XXXXXXXXXXS11XXXS15XXXXXXXXXX |
| Oligonucleotide | positions 11 and | | |
| with Spacers at | 15 are | | |
| positions 11 and 15 | substituted with | | |
| (SMN2 AON | spacers | | |
| 25_sp11sp15) | | | |
| SMN2 | Nucleosides at | 10594 | XXXXXXXS8XXXXXXXS16XXXXXXXXX |
| Oligonucleotide | positions 8 and | | |
| with Spacers at | 16 are | | |
| positions 8 and 16 | substituted with | | |
| (SMN2 AON | spacers | | |
| 25_sp8sp16) | | | |
| SMN2 | Nucleosides at | 10595 | XXXXXXXXS9XXXXXXS16XXXXXXXXX |
| Oligonucleotide | positions 9 and | | |
| with Spacers at | 16 are | | |
| positions 9 and 16 | substituted with | | |
| (SMN2 AON | spacers | | |
| 25_sp9sp16) | | | |
| SMN2 | Nucleosides at | 10596 | XXXXXXXXXS10XXXXXS16XXXXXXXXX |
| Oligonucleotide | positions 10 and | | |
| with Spacers at | 16 are | | |
| positions 10 and 16 | substituted with | | |
| (SMN2 AON | spacers | | |
| 25_sp10sp16) | | | |
| SMN2 | Nucleosides at | 10597 | XXXXXXXXXXS11XXXXS16XXXXXXXXX |
| Oligonucleotide | positions 11 and | | |
| with Spacers at | 16 are | | |
| positions 11 and 16 | substituted with | | |
| (SMN2 AON | spacers | | |
| 25_spl1sp16) | | | |
| SMN2 | Nucleosides at | 10598 | XXXXXXXS8XXXXXXXXS17XXXXXXXX |
| Oligonucleotide | positions 8 and | | |
| with Spacers at | 17 are | | |
| positions 8 and 17 | substituted with | | |
| (SMN2 AON | spacers | | |
| 25_sp8sp17) | | | |
| SMN2 | Nucleosides at | 10599 | XXXXXXXXS8XXXXXXXS17XXXXXXXX |
| Oligonucleotide | positions 9 and | | |
| with Spacers at | 17 are | | |
| positions 9 and 17 | substituted with | | |
| (SMN2 AON | spacers | | |
| 25_sp9sp17) | | | |
| SMN2 | Nucleosides at | 10600 | XXXXXXXXXS10XXXXXXS17XXXXXXXX |
| Oligonucleotide | positions 10 and | | |
| with Spacers at | 17 are | | |
| positions 10 and 17 | substituted with | | |
| (SMN2 AON | spacers | | |
| 25_sp10sp17) | | | |
| SMN2 | Nucleosides at | 10601 | XXXXXXXXXXS11XXXXXS17XXXXXXXX |
| Oligonucleotide | positions 11 and | | |
| with Spacers at | 17 are | | |
| positions 11 and 17 | substituted with | | |
| (SMN2 AON | spacers | | |
| 25_sp11sp17) | | | |
| SMN2 | Nucleosides at | 10602 | XXXXXXXS8XXXXXXXXXS18XXXXXXX |
| Oligonucleotide | positions 8 and | | |
| with Spacers at | 18 are | | |
| positions 8 and 18 | substituted with | | |
| (SMN2 ATON | spacers | | |
| 25_sp8sp18) | | | |
| SMN2 | Nucleosides at | 10603 | XXXXXXXXS9XXXXXXXXS18XXXXXXX |
| Oligonucleotide | positions 9 and | | |
| with Spacers at | 18 are | | |
| positions 9 and 18 | substituted with | | |
| (SMN2 AON | spacers | | |
| 25_sp9sp18) | | | |
| SMN2 | Nucleosides at | 10604 | XXXXXXXXXS10XXXXXXXS18XXXXXXX |
| Oligonucleotide | positions 10 and | | |
| with Spacers at | 18 are | | |
| positions 10 and 18 | substituted with | | |
| (SMN2 AON | spacers | | |
| 25_sp10sp18) | | | |
| SMN2 | Nucleosides at | 10605 | XXXXXXXXXXS11XXXXXXS18XXXXXXX |
| Oligonucleotide | positions 11 and | | |
| with Spacers at | 18 are | | |
| positions 11 and 18 | substituted with | | |
| (SMN2 AON | spacers | | |
| 25_sp11sp18) | | | |
| SMN2 | Nucleosides at | 10606 | XXXXXXXXS9XXXXXXXXXS19XXXXXX |
| Oligonucleotide | positions 9 and | | |
| with Spacers at | 19 are | | |
| positions 9 and 19 | substituted with | | |
| (SMN2 AON | spacers | | |
| 25_sp9sp19) | | | |
| SMN2 | Nucleosides at | 10607 | XXXXXXXXXS10XXXXXXXXS19XXXXXX |
| Oligonucleotide | positions 10 and | | |
| with Spacers at | 19 are | | |
| positions 10 and 19 | substituted with | | |
| (SMN2 AON | spacers | | |
| 25_sp10sp19) | | | |
| SMN2 | Nucleosides at | 10608 | XXXXXXXXXXS11XXXXXXXS19XXXXXX |
| Oligonucleotide | positions 11 and | | |
| with Spacers at | 19 are | | |
| positions 11 and 19 | substituted with | | |
| (SMN2 AON | spacers | | |
| 25_sp11sp19) | | | |
| SMN2 | Nucleosides at | 10609 | XXXXXXXXS9XXXXXXXXXXS20XXXXX |
| Oligonucleotide | positions 9 and | | |
| with Spacers at | 20 are | | |
| positions 9 and 20 | substituted with | | |
| (SMN2 AON | spacers | | |
| 25_sp9sp20) | | | |
| SMN2 | Nucleosides at | 10610 | XXXXXXXXXS10XXXXXXXXXS20XXXXX |
| Oligonucleotide | positions 10 and | | |
| with Spacers at | 20 are | | |
| positions 10 and 20 | substituted with | | |
| (SMN2 AON | spacers | | |
| 25_sp10sp20) | | | |
| SMN2 | Nucleosides at | 10611 | XXXXXXXXXXS11XXXXXXXXS20XXXXX |
| Oligonucleotide | positions 11 and | | |
| with Spacers at | 20 are | | |
| positions 11 and 20 | substituted with | | |
| (SMN2 AON | spacers | | |
| 25_sp11sp20) | | | |
| SMN2 | Nucleosides at | 10612 | XXXXXXXXXS10XXXXXXXXXXS21XXXX |
| Oligonucleotide | positions 10 and | | |
| with Spacers at | 21 are | | |
| positions 10 and 21 | substituted with | | |
| (SMN2 AON | spacers | | |
| 25_sp10sp21) | | | |
| SMN2 | Nucleosides at | 10613 | XXXXXXXXXXS11XXXXXXXXXS21XXXX |
| Oligonucleotide | positions 11 and | | |
| with Spacers at | 21 are | | |
| positions 11 and 21 | substituted with | | |
| (SMN2 AON | spacers | | |
| 25_sp11sp21) | | | |
| SMN2 | Nucleosides at | 10614 | XXXS4XXXXXXXXXXS15XXXXXXXXXX |
| Oligonucleotide | positions 4 and | | |
| with Spacers at | 15 are | | |
| positions 4 and 15 | substituted with | | |
| (SMN2 AON | spacers | | |
| 25_sp4sp15) | | | |
| SMN2 | Nucleosides at | 10615 | XXXXXXS7XXXXXXXXXXXS19XXXXXX |
| Oligonucleotide | positions 7 and | | |
| with Spacers at | 19 are | | |
| positions 7 and 19 | substituted with | | |
| (SMN2 AON | spacers | | |
| 25_sp7sp19) | | | |
| SMN2 | Nucleosides at | 10616 | XXXXXXS7XXXXXXXXXXS18XXXXXXX |
| Oligonucleotide | positions 7 and | | |
| with Spacers at | 18 are | | |
| positions 7 and 18 | substituted with | | |
| (SMN2 AON | spacers | | |
| 25_sp7sp18) | | | |
| SMN2 | Nucleosides at | 10617 | XXXXXXXXS9XXXXXXXXXXXS21XXXX |
| Oligonucleotide | positions 9 and | | |
| with Spacers at | 21 are | | |
| positions 9 and 21 | substituted with | | |
| (SMN2 AON | spacers | | |
| 25_sp9sp21) | | | |

*At least one nucleoside linkage of the nucleobase sequence is selected from a phosphorothioate
linkage, an alkyl phosphate linkage, a phosphorodithioate linkage, a phosphotriester linkage, an
alkylphosphonate linkage, a 3-methoxypropyl phosphonate linkage, a methylphosphonate
linkage, an aminoalkylphosphotriester linkage, an alkylene phosphonate linkage, a phosphinate
linkage, a phosphoramidate linkage, a phosphoramidothioate linkage, a phosphorodiamidate
(e.g., comprising a phosphorodiamidate morpholino (PMO), 3′ amino ribose, or 5′ amino ribose)
linkage, an aminoalkylphosphoramidate linkage, a thiophosphoramidate linkage, a
thionoalkylphosphonate linkage, a thionoalkylphosphotriester linkage, a thiophosphate linkage, a
selenophosphate linkage, and a boranophosphate linkage.

In various embodiments, SMN2 oligonucleotides include three spacers. The inclusion of three spacers divides up the SMN2 oligonucleotide into four separate segments. In various embodiments, the three spacers are located at different positions of the SMN2 oligonucleotide such that each of the segments of the SMN2 oligonucleotide are at most 7 linked nucleosides in length. Example SMN2 AONs with three spacers are documented below in Table 23.

TABLE 23
Identification of SMN2 AONs or AON variants with three spacers. Here, each SMN2
AON has 4 segments, where each segment has at most 7 linked nucleosides.

| | | | Sequence* (where X indicates a |
|---|---|---|---|
| | | | nucleoside of the SMN2 parent |
| | Relation to | Sequence ID | oligonucleotide and S_{y} indicates |
| | SMN2 parent | Number | presence of a Spacer where y denotes |
| Sequence name | oligonucleotide | (SEQ ID NO) | the position) (5′ → 3′) |
| SMN2 | Nucleosides | 10618 | XXXXXXXS8XXXXXXXS16XXXXXXXS24X |
| Oligonucleotide | at positions 8 | | |
| with Spacers at | and 16 and 24 | | |
| positions 8 and 16 | are substituted | | |
| and 24 (SMN2 | with spacers | | |
| AON | | | |
| 25_sp8sp16sp24) | | | |
| SMN2 | Nucleosides | 10619 | XXXXXXXS8XXXXXXXS16XXXXXXS23XX |
| Oligonucleotide | at positions 8 | | |
| with Spacers at | and 16 and 23 | | |
| positions 8 and 16 | are substituted | | |
| and 23 (SMN2 | with spacers | | |
| AON | | | |
| 25_sp8sp16sp23) | | | |
| SMN2 | Nucleosides | 10620 | XS2XXXXXXXS10XXXXXXXS18XXXXXXX |
| Oligonucleotide | at positions 2 | | |
| with Spacers at | and 10 and 18 | | |
| positions 2 and 10 | are substituted | | |
| and 18 (SMN2 | with spacers | | |
| AON | | | |
| 25_sp8sp16sp23) | | | |
| SMN2 | Nucleosides | 10621 | XXS3XXXXXXS10XXXXXXXS18XXXXX |
| Oligonucleotide | at positions 3 | | |
| with Spacers at | and 10 and 18 | | |
| positions 3 and 10 | are substituted | | |
| and 18 (SMN2 | with spacers | | |
| AON | | | |
| 25_sp8sp16sp23) | | | |
| SMN2 | Nucleosides | 10622 | XXXS4XXXXXXXS12XXXXXXS19XXXXXX |
| Oligonucleotide | at positions 4 | | |
| with Spacers at | and 12 and 19 | | |
| positions 4 and 12 | are substituted | | |
| and 19 (SMN2 | with spacers | | |
| AON | | | |
| 25_sp4sp12sp19) | | | |
| SMN2 | Nucleosides | 10623 | XXXXXXXS8XXXXS13XXXXS18XXXXXXX |
| Oligonucleotide | at positions 8 | | |
| with Spacers at | and 13 and 18 | | |
| positions 8 and 13 | are substituted | | |
| and 18 (SMN2 | with spacers | | |
| AON | | | |
| 25_sp8sp13sp18) | | | |
| SMN2 | Nucleosides | 10624 | XXXXS5XXXXXXXS13XXXXXXXS21XXXX |
| Oligonucleotide | at positions 5 | | |
| with Spacers at | and 13 and 21 | | |
| positions 5 and 13 | are substituted | | |
| and 21 (SMN2 | with spacers | | |
| AON | | | |
| 25_sp5sp13sp21) | | | |
| SMN2 | Nucleosides | 10625 | XXXXXXS7XXXXXS13XXXXXS19XXXXXX |
| Oligonucleotide | at positions 7 | | |
| with Spacers at | and 13 and 19 | | |
| positions 7 and 13 | are substituted | | |
| and 19 (SMN2 | with spacers | | |
| AON | | | |
| 25_sp7sp13sp19) | | | |
| SMN2 | Nucleosides | 10626 | XXXXXS8XXXXXXS13XXXXXXS20XXXXX |
| Oligonucleotide | at positions 6 | | |
| with Spacers at | and 13 and 20 | | |
| positions 6 and 13 | are substituted | | |
| and 20 (SMN2 | with spacers | | |
| AON | | | |
| 25_sp6sp13sp20) | | | |
| SMN2 | Nucleosides | 10627 | XXXXXXXS8XXS11XXXXXXXS19XXXXXX |
| Oligonucleotide | at positions 8 | | |
| with Spacers at | and 11 and 19 | | |
| positions 8 and 11 | are substituted | | |
| and 19 (SMN2 | with spacers | | |
| AON | | | |
| 25_sp8sp11sp19) | | | |
| SMN2 | Nucleosides | 10628 | XXXXXXXS8XXS11XXXXS16XXXXXXX |
| Oligonucleotide | at positions 8 | | |
| with Spacers at | and 11 and 16 | | |
| positions 8 and 11 | are substituted | | |
| and 16 (SMN2 | with spacers | | |
| AON | | | |
| 23_sp8sp11sp16) | | | |
| SMN2 | Nucleosides | 10629 | XXXXXXS7XXXXXXS14XXXXXXXS22XXX |
| Oligonucleotide | at positions 7 | | |
| with Spacers at | and 14 and 22 | | |
| positions 7 and 14 | are substituted | | |
| and 22 (SMN2 | with spacers | | |
| AON | | | |
| 23_sp7sp14sp22) | | | |

*At least one nucleoside linkage of the nucleobase sequence is selected from a phosphorothioate
linkage, an alkyl phosphate linkage, a phosphorodithioate linkage, a phosphotriester linkage, an
alkylphosphonate linkage, a 3-methoxypropyl phosphonate linkage, a methylphosphonate
linkage, an aminoalkylphosphotriester linkage, an alkylene phosphonate linkage, a phosphinate
linkage, a phosphoramidate linkage, a phosphoramidothioate linkage, a phosphorodiamidate
(e.g., comprising a phosphorodiamidate morpholino (PMO), 3′ amino ribose, or 5′ amino ribose)
linkage, an aminoalkylphosphoramidate linkage, a thiophosphoramidate linkage, a
thionoalkylphosphonate linkage, a thionoalkylphosphotriester linkage, a thiophosphate linkage, a
selenophosphate linkage, and a boranophosphate linkage.

In various embodiments, SMN2 AONs with one or more spacers are reduced in length in comparison to the SMN2 AONs described above in Tables 22B and 23. For example, such SMN2 AONs may be SMN2 oligonucleotide variants with one or more spacers. In various embodiments, the SMN2 oligonucleotide variants with one or more spacers are 23mers, 21mers, or 19mers. In various embodiments, SMN2 oligonucleotide variants include two spacers such that the SMN2 oligonucleotide variant includes three segments that are divided up by the two spacers. In various embodiments, at least one of the three segments has at most 7 linked nucleosides. In various embodiments, each of the three segments has at most 7 linked nucleosides. Example SMN2 oligonucleotide variants with one or more spacers are shown below in Table 24.

TABLE 24
SMN2 AON variants with two spacers. Here, each SMN2 AON variant has 3 segments.

| | | | Sequence* (where X indicates a |
|---|---|---|---|
| | | | nucleoside of the SMN2 oligonucleotide |
| | Relation to SMN2 | Sequence ID | variant and S_{y} indicates presence of a |
| | oligonucleotide | Number | Spacer where _{y} denotes the position) |
| Sequence name | variant | (SEQ ID NO) | (5′ → 3′) |
| SMN2 | N/A | 10630 | XXXXXXXXXXXXXXXXXXXXXXX (23mer) |
| oligonucleotide | | | |
| variant (23mer) | | | |
| SMN2 Variant | Nucleosides at | 10631 | XXXXXXXS8XXXXXXXS16XXXXXXX |
| Oligonucleotide | positions 8 and | | |
| (23mer) with | 16 are | | |
| Spacers at | substituted with | | |
| positions 8 and | spacers | | |
| 16 (SMN2 | | | |
| AON variant | | | |
| 23_sp8sp16) | | | |
| SMN2 | N/A | 10632 | XXXXXXXXXXXXXXXXXXXXX (21mer) |
| oligonucleotide | | | |
| variant (21mer) | | | |
| SMN2 Variant | Nucleosides at | 10633 | XXXXS5XXXXXXS12XXXXXXXXX |
| Oligonucleotide | positions 5 and | | |
| (21mer) with | 12 are | | |
| Spacers at | substituted with | | |
| positions 5 and | spacers | | |
| 12 (SMN2 | | | |
| AON variant | | | |
| 21_sp5sp12) | | | |
| SMN2 Variant | Nucleosides at | 10634 | XXXXXXXS8XXXXXXXS16XXXXX |
| Oligonucleotide | positions 8 and | | |
| (21mer) with | 16 are | | |
| Spacers at | substituted with | | |
| positions 8 and | spacers | | |
| 16 (SMN2 | | | |
| AON variant | | | |
| 21_sp8sp16) | | | |
| SMN2 Variant | Nucleosides at | 10635 | XXXXXS6XXXXXXXS14XXXXXXX |
| Oligonucleotide | positions 6 and | | |
| (21mer) with | 14 are | | |
| Spacers at | substituted with | | |
| positions 6 and | spacers | | |
| 14 (SMN2 | | | |
| AON variant | | | |
| 21_sp6sp14) | | | |
| SMN2 Variant | Nucleosides at | 10636 | XXXXXXXS8XXXXXS14XXXXXXX |
| Oligonucleotide | positions 8 and | | |
| (21mer) with | 14 are | | |
| Spacers at | substituted with | | |
| positions 8 and | spacers | | |
| 14 (SMN2 | | | |
| AON variant | | | |
| 21_sp8sp14) | | | |
| SMN2 Variant | Nucleosides at | 10637 | XXXXXS6XXXXXXXXXXXXXS20X |
| Oligonucleotide | positions 6 and | | |
| (21mer) with | 20 are | | |
| Spacers at | substituted with | | |
| positions 8 and | spacers | | |
| 14 (SMN2 | | | |
| AON variant | | | |
| 21_sp8sp14) | | | |
| SMN2 | N/A | 10638 | XXXXXXXXXXXXXXXXXXX (19mer) |
| oligonucleotide | | | |
| variant (19mer) | | | |
| SMN2 Variant | Nucleosides at | 10639 | XXXXS5XXXXXXS12XXXXXXX |
| Oligonucleotide | positions 5 and | | |
| (19mer) with | 12 are | | |
| Spacers at | substituted with | | |
| positions 5 and | spacers | | |
| 12 (SMN2 | | | |
| AON variant | | | |
| 19_sp5sp12) | | | |
| SMN2 Variant | Nucleosides at | 10640 | XXXXXXXS8XXXXXXS15XXXX |
| Oligonucleotide | positions 8 and | | |
| (19mer) with | 15 are | | |
| Spacers at | | | |
| positions 8 and | substituted with | | |
| 15 (SMN2 | spacers | | |
| AON variant | | | |
| 19_sp8sp15) | | | |

*At least one nucleoside linkage of the nucleobase sequence is selected from a phosphorothioate
linkage, an alkyl phosphate linkage, a phosphorodithioate linkage, a phosphotriester linkage, an
alkylphosphonate linkage, a 3-methoxypropyl phosphonate linkage, a methylphosphonate
linkage, an aminoalkylphosphotriester linkage, an alkylene phosphonate linkage, a phosphinate
linkage, a phosphoramidate linkage, a phosphoramidothioate linkage, a phosphorodiamidate
(e.g., comprising a phosphorodiamidate morpholino (PMO), 3′ amino ribose, or 5′ amino ribose)
linkage, an aminoalkylphosphoramidate linkage, a thiophosphoramidate linkage, a
thionoalkylphosphonate linkage, a thionoalkylphosphotriester linkage, a thiophosphate linkage, a
selenophosphate linkage, and a boranophosphate linkage.
)

In some embodiments, an antisense oligonucleotide disclosed herein (e.g., SMN2 parent oligonucleotides and/or SMN2 oligonucleotide variants) comprise one or more spacers as well as one or more locked nucleic acids (LNAs). In some embodiments, an antisense oligonucleotide disclosed herein (e.g., SMN2 parent oligonucleotides and/or SMN2 oligonucleotide variants) comprises two spacers and two LNAs. In some embodiments, an antisense oligonucleotide disclosed herein (e.g., SMN2 parent oligonucleotides and/or SMN2 oligonucleotide variants) comprises two spacers and three LNAs.

In various embodiments, a spacer and a LNA are located adjacent to one another in an antisense oligonucleotide. For example, if counting from 5′ to 3′, a LNA can be located at a position of the antisense oligonucleotide and a spacer can be located at a 8^{th} position of the antisense oligonucleotide. As another example, if counting from 5′ to 3′, a LNA can be located at a 9^{th} position of the antisense oligonucleotide and a spacer can be located at a 8^{th} position of the antisense oligonucleotide. As another example, if counting from 5′ to 3′, a LNA can be located at a 15^{th} position of the antisense oligonucleotide and a spacer can be located at a 16^{th} position of the antisense oligonucleotide. As another example, if counting from 5′ to 3′, a LNA can be located at a 17^{th} position of the antisense oligonucleotide and a spacer can be located at a 16^{th} position of the antisense oligonucleotide.

In particular embodiments, a first spacer is located adjacent to a first LNA and a second spacer is located adjacent to a second LNA in an antisense oligonucleotide. For example, if counting from 5′ to 3′, a first LNA can be located at a 7^{th} position of the antisense oligonucleotide, a first spacer can be located at a 8^{th} position of the antisense oligonucleotide, a second LNA can be located at a 15^{th} position of the antisense oligonucleotide, and a second spacer can be located at a 16^{th} position of the antisense oligonucleotide. As another example, if counting from 5′ to 3′, a first LNA can be located at a 7^{th} position of the antisense oligonucleotide, a first spacer can be located at a 8^{th} position of the antisense oligonucleotide, a second LNA can be located at a 17^{th} position of the antisense oligonucleotide, and a second spacer can be located at a 16^{th} position of the antisense oligonucleotide. As another example, if counting from 5′ to 3′, a first LNA can be located at a 9^{th} position of the antisense oligonucleotide, a first spacer can be located at a 8^{th} position of the antisense oligonucleotide, a second LNA can be located at a 17^{th} position of the antisense oligonucleotide, and a second spacer can be located at a 16^{th} position of the antisense oligonucleotide.

In various embodiments, one or more spacers and one or more LNAs are not located adjacent to one another in an antisense oligonucleotide. For example, if counting from 5′ to 3′, a LNA can be located at a 4^{th} position of the antisense oligonucleotide and a spacer can be located at a 8^{th} position of the antisense oligonucleotide. As example, if counting from 5′ to 3′, a LNA can be located at a 20^{th} position of the antisense oligonucleotide and a spacer can be located at a 16^{th} position of the antisense oligonucleotide. In particular embodiments, if counting from 5′ to 3′, a first LNA can be located at a 4^{th} position of the antisense oligonucleotide, a first spacer can be located at a 8^{th} position of the antisense oligonucleotide, a second LNA can be located at a 20¹ position of the antisense oligonucleotide, and a second spacer can be located at a 16^{th} position of the antisense oligonucleotide. In particular embodiments, if counting from 5′ to 3′, a first LNA can be located at a 4^{th} position of the antisense oligonucleotide, a first spacer can be located at a 8^{th} position of the antisense oligonucleotide, a second LNA can be located at a 12^{th} position of the antisense oligonucleotide, a second spacer can be located at a 16^{th} position of the antisense oligonucleotide, and a third LNA can be located at a 20^{th} position of the antisense oligonucleotide.

### Example Synthesis of Spacer Building Blocks for Spacers

Additionally disclosed herein are compositions and methods for synthesizing a spacer, such as a spacer of any of Formulae (I), (I′), (Ia), (Ia′), (II), (II′), (Iia), (Iia′), (IIi), (IIi′), (IIib), (IIib′), (III), (III′), (IIIa) and (IIIa′). In particular embodiments, compositions and methods disclosed herein are useful for synthesizing an AON including a spacer of Formulae (IIib) or (IIib′).

In various embodiments, the methods for synthesizing a spacer of Formulae (IIib) or (IIib′) uses a 5 step synthesis protocol to generate a spacer building block. An example 5 step synthesis protocol is shown below. In particular the 5 step synthesis protocol starts with a Compound A, the generation of intermediate compounds (e.g., Compound B, Compound C, Compound D, and compound E), and finally resulting in Compound F. Here, compound F represents a spacer building block for synthesizing an AON including a spacer of Formulae (IIib) or (IIib′).

In various embodiments, step 1 involves a benzoyl protection step. In various embodiments, step 2 involves a reductive nucleobase cleavage step. In various embodiments, step 3 involves a saponification step. In various embodiments, step 4 involves a DMT protection step. In various embodiments, step 5 involves a phosphoramidite installation step.

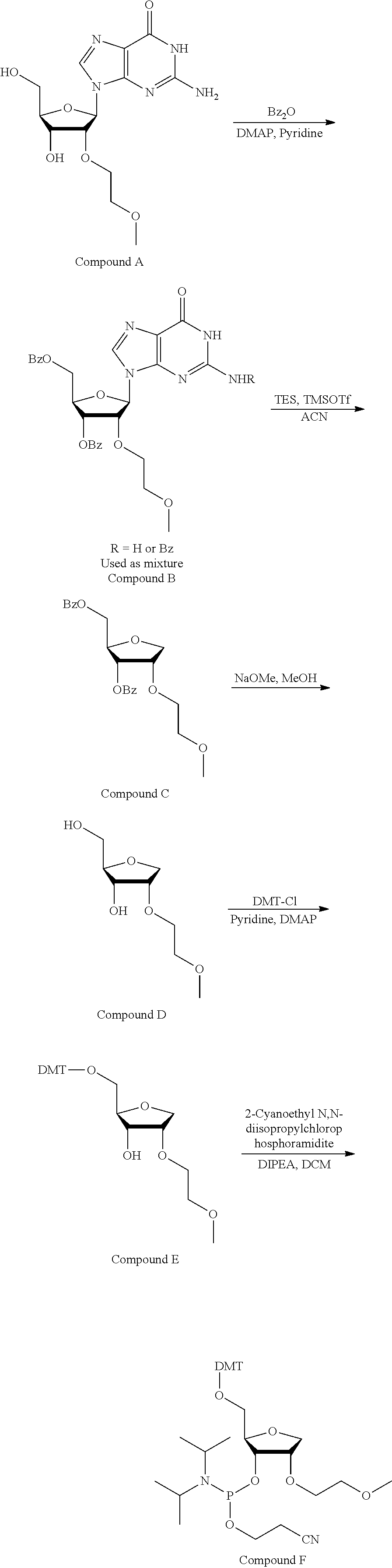

### Performance of STMN2 Oligonucleotides

Generally, STMN2 oligonucleotides and/or STMN2 parent oligonucleotides (e.g., STMN2 oligonucleotides with sequences of any of SEQ ID NOs: 1-466, SEQ ID NO: 893-1338, SEQ ID NOs: 1342-1366, SEQ ID NOs: 1392-1664, and SEQ ID NOs: 10655-10669) target STMN2 transcripts (for example, a STMN2 pre-miRNA comprising a cryptic exon) comprising a sequence that shares at least 90% (e.g., 90%, 91%, 92%, 93%, 94%, 95%, 96%, 97%, 98%, 99%, or 100%) identity to SEQ ID NO: 1339 or SEQ ID NO: 1341 in order to increase, restore, rescue, or stabilize levels of expression of STMN2 mRNA that is capable of translation to produce a functional STMN2 protein (e.g., full length STMN2). In various embodiments, STMN2 AONs can exhibit at least a 20%, 30%, 40%, 50%, 60%, 70%, 80%, or 90% increase of full length STMN2 protein. In various embodiments, STMN2 AONs can exhibit at least a 100%, 200%, 300%, or 400% increase of full length STMN2 protein. In some embodiments, the percent increase of the full length STMN2 protein is an increase in comparison to a reduced level of full length STMN2 protein achieved using a TDP43 antisense oligonucleotide. For example, a TDP43 antisense oligonucleotide can be used to deplete full length STMN2 protein followed by increase of the full length STMN2 protein using a STMN2 AON.

In some embodiments, STMN2 AONs can exhibit at least a 10%, 20%, 30%, 40%, 50%, 60%, 70%, 80%, 90%, or 100% rescue of full length STMN2 protein. In some embodiments, the percent rescue of full length STMN2 refers to the % of full length STMN2 following depletion using a TDP43 antisense oligonucleotide and a treatment using STMN2 AONs in comparison to a negative control (e.g., cells that did not undergo depletion or treatment or cells that were treated with a vehicle solution).

### Performance of KCNQ2 Oligonucleotides

Generally, KCNQ2 oligonucleotides and/or KCNQ2 parent oligonucleotides (e.g., KCNQ2 oligonucleotides with sequences of any of SEQ ID NOs: 1676-1851, SEQ ID NOs: 2028-2529, SEQ ID NOs: 3046-3221, SEQ ID NOs: 3398-3899, and SEQ ID NOs: 4402-4530) target KCNQ2 transcripts (for example, a KCNQ2 mRNA comprising a sequence that shares at least 90% (e.g., 90%, 91%, 92%, 93%, 94%, 95%, 96%, 97%, 98%, 99%, or 100%) identity to any one of SEQ ID NO: 3032-3043 in order to increase, restore, rescue, or stabilize levels of expression of KCNQ2 mRNA that is capable of translation to produce a functional KCNQ2 protein (e.g., full length KCNQ2). In various embodiments, KCNQ2 AONs can exhibit at least a 20%, 30%, 40%, 50%, 60%, 70%, 80%, or 90% increase of full length KCNQ2 mRNA. In various embodiments, KCNQ2 AONs can exhibit at least a 100%, 200%, 300%, or 400% increase of full length KCNQ2 mRNA. In various embodiments, KCNQ2 AONs can exhibit at least a 20%, 30%, 40%, 50%, 60%, 70%, 80%, or 90% reduction of mis-spliced KCNQ2 mRNA. In various embodiments, KCNQ2 AONs can exhibit at least a 20%, 30%, 40%, 50%, 60%, 70%, 80%, or 90% increase of full length KCNQ2 protein. In various embodiments, KCNQ2 AONs can exhibit at least a 100%, 200%, 300%, or 400% increase of full length KCNQ2 protein. In some embodiments, the percent increase of the full length KCNQ2 protein is an increase in comparison to a reduced level of full length KCNQ2 protein achieved using a KCNQ2 antisense oligonucleotide. For example, a KCNQ2 antisense oligonucleotide can be used to deplete full length KCNQ2 protein followed by increase of the full length KCNQ2 protein using a KCNQ2 AON.

In some embodiments, KCNQ2 AONs can exhibit at least a 10%, 20%, 30%, 40%, 50%, 60%, 70%, 80%, 90%, or 100% rescue of full length KCNQ2 protein. In some embodiments, the percent rescue of full length KCNQ2 refers to the % of full length KCNQ2 following depletion using a TDP43 antisense oligonucleotide and a treatment using KCNQ2 AONs in comparison to a negative control (e.g., cells that did not undergo depletion or treatment or cells that were treated with a vehicle solution).

### Performance of UNC13A Oligonucleotides

Generally, UNC13A oligonucleotides and/or UNC13A parent oligonucleotides (e.g., UNC13A oligonucleotides with sequences of any of SEQ ID NOs: 4531-5794, SEQ ID NO: 7059-8322, SEQ ID NOs: 9596-9696, or SEQ ID NOs: 10670-10779) target UNC13A transcripts (for example, a UNC13A pre-mRNA) comprising a sequence that shares at least 90% (e.g., 90%, 91%, 92%, 93%, 94%, 95%, 96%, 97%, 98%, 99%, or 100%) identity to SEQ ID NO: 9587-9595 in order to increase, restore, rescue, or stabilize levels of expression of UNC13A mRNA that is capable of translation to produce a functional UNC13A protein (e.g., full length UNC13A). In various embodiments, UNC13A AONs can exhibit at least a 20%, 30%, 40%, 50%, 60%, 70%, 80%, or 90% increase of full length UNC13A mRNA. In various embodiments, UNC13A AONs can exhibit at least a 100%, 200%, 300%, or 400% increase of full length UNC13A mRNA. In various embodiments, UNC13A AONs can exhibit at least a 20%, 30%, 40%, 50%, 60%, 70%, 80%, or 90% reduction of mis-spliced UNC13A mRNA. In various embodiments, UNC13A AONs can exhibit at least a 20%, 30%, 40%, 50%, 60%, 70%, 80%, or 90% increase of full length UNC13A protein. In various embodiments, UNC13A AONs can exhibit at least a 100%, 200%, 300%, or 400% increase of full length UNC13A protein. In some embodiments, the percent increase of the full length UNC13A protein is an increase in comparison to a reduced level of full length UNC13A protein achieved using a TDP43 antisense oligonucleotide. For example, a TDP43 antisense oligonucleotide can be used to deplete full length UNC13A protein followed by increase of the full length UNC13A protein using a UNC13A AON.

In some embodiments, UNC13A AONs can exhibit at least a 10%, 20%, 30%, 40%, 50%, 60%, 70%, 80%, 90%, or 100% rescue of full length UNC13A protein. In some embodiments, the percent rescue of full length UNC13A refers to the % of full length UNC13A following depletion using a TDP43 antisense oligonucleotide and a treatment using UNC13A AONs in comparison to a negative control (e.g., cells that did not undergo depletion or treatment or cells that were treated with a vehicle solution).

### Performance of SMN2 Oligonucleotides

Generally, SMN2 oligonucleotides and/or SMN2 parent oligonucleotides (e.g., SMN2 oligonucleotides with sequences of any of SEQ ID NOs: 9710-10141 and SEQ ID NOs: 10574-10581) target SMN2 transcripts (for example, a SMN2 mRNA comprising a sequence that shares at least 90% (e.g., 90%, 91%, 92%, 93%, 94%, 95%, 96%, 97%, 98%, 99%, or 100%) identity to any one of SEQ ID NO: 9698-9707 in order to increase, restore, rescue, or stabilize levels of expression of SMN2 mRNA that is capable of translation to produce a functional SMN2 protein (e.g., full length SMN2). In various embodiments, SMN2 AONs can exhibit at least a 20%, 30%, 40%, 50%, 60%, 70%, 80%, or 90% increase of full length SMN2 protein. In various embodiments, SMN2 AONs can exhibit at least a 100%, 200%, 300%, or 400% increase of full length SMN2 protein. In some embodiments, the percent increase of the full length SMN2 protein is an increase in comparison to a reduced level of full length SMN2 protein achieved using a SMN2 antisense oligonucleotide. For example, a SMN2 antisense oligonucleotide can be used to deplete full length SMN2 protein followed by increase of the full length SMN2 protein using a SMN2 AON.

In some embodiments, SMN2 AONs can exhibit at least a 10%, 20%, 30%, 40%, 50%, 60%, 70%, 80%, 90%, or 100% rescue of full length SMN2 protein. In some embodiments, the percent rescue of full length SMN2 refers to the % of full length SMN2 following depletion using a TDP43 antisense oligonucleotide and a treatment using SMN2 AONs in comparison to a negative control (e.g., cells that did not undergo depletion or treatment or cells that were treated with a vehicle solution).

### Modifications

A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to antisense compounds encompass substitutions or changes to internucleoside linkages, sugar moieties, or nucleobases. Modified antisense compounds are often preferred over native forms because of desirable properties such as, for example, enhanced cellular uptake, enhanced affinity for nucleic acid target, increased stability in the presence of nucleases, or increased activity.

Chemically modified nucleosides may also be employed to increase the binding affinity of a shortened or truncated antisense oligonucleotide for its target nucleic acid. Consequently, comparable results can often be obtained with shorter antisense compounds that have such chemically modified nucleosides.

### Modified Internucleoside Linkages

The naturally occurring internucleoside linkage of RNA and DNA is a 3′ to 5′ phosphodiester linkage. Antisense compounds having one or more modified, i.e. non-naturally occurring, internucleoside linkages are often selected over antisense compounds having naturally occurring internucleoside linkages because of desirable properties such as, for example, enhanced cellular uptake, enhanced affinity for target nucleic acids, and increased stability in the presence of nucleases.

Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, antisense compounds targeted to a STMN2 nucleic acid comprise one or more modified internucleoside linkages. In certain embodiments, antisense compounds targeted to a KCNQ2 nucleic acid comprise one or more modified internucleoside linkages. In certain embodiments, antisense compounds targeted to a UNC13A nucleic acid comprise one or more modified internucleoside linkages. In certain embodiments, antisense compounds targeted to a SMN2 nucleic acid comprise one or more modified internucleoside linkages. In certain embodiments, the modified internucleoside linkages are interspersed throughout the antisense compound. In certain embodiments, the modified internucleoside linkages are phosphorothioate linkages. In certain embodiments, each internucleoside linkage of an antisense compound is a phosphorothioate internucleoside linkage. In certain embodiments, the antisense compounds targeted to a STMN2 nucleic acid comprise at least one phosphodiester linkage and at least one phosphorothioate linkage. In certain embodiments, the antisense compounds targeted to a KCNQ2 nucleic acid comprise at least one phosphodiester linkage and at least one phosphorothioate linkage. In certain embodiments, the antisense compounds targeted to a UNC13A nucleic acid comprise at least one phosphodiester linkage and at least one phosphorothioate linkage. In certain embodiments, the antisense compounds targeted to a SMN2 nucleic acid comprise at least one phosphodiester linkage and at least one phosphorothioate linkage.

### Modified Sugar Moieties

Antisense compounds can optionally contain one or more nucleosides wherein the sugar group has been modified. Such sugar modified nucleosides may impart enhanced nuclease stability, increased binding affinity, or some other beneficial biological property to the antisense compounds. In certain embodiments, nucleosides comprise chemically modified ribofuranose ring moieties. Examples of chemically modified ribofuranose rings include without limitation, addition of substituent groups (including 5′ and 2′ substituent groups, bridging of non-geminal ring atoms to form bicyclic nucleic acids (BNA), replacement of the ribosyl ring oxygen atom with S, N(R) or C(R₁)(R₂) (R, R₁ and R₂ are each independently H, C₁-C₁₂ alkyl or a protecting group) and combinations thereof. Examples of chemically modified sugars include 2′-F-5′-methyl substituted nucleoside (see PCT International Application WO 2008/101157 Published on Aug. 21, 2008 for other disclosed 5′,2′-bis substituted nucleosides) or replacement of the ribosyl ring oxygen atom with S with further substitution at the 2′-position (see published U.S. Patent Application US2005-0130923, published on Jun. 16, 2005) or alternatively 5′-substitution of a BNA (see PCT International Application WO 2007/134181 Published on Nov. 22, 2007 wherein LNA is substituted with for example a 5′-methyl or a 5′-vinyl group).

Examples of nucleosides having modified sugar moieties include without limitation nucleosides comprising 5′-vinyl, 5′-methyl (R or 5), 4′-S, 2′-F, 2′-OCH₃, 2′-OCH₂CH₃, 2′-O CH₂ CH₂F and 2′-O(CH₂)₂OCH₃ substituent groups. The substituent at the 2′ position can also be selected from allyl, amino, azido, thio, O-allyl, O—C₁-C₁₀ alkyl, OCF₃, OCH₂F, O(CH₂)₂S CH₃, O(CH₂)₂—O—N(Rₘ)(Rₙ), O—CH₂—C(═O)—N(Rₘ)(Rₙ), and O—CH₂—C(═O)—N(Rₗ)—(CH₂)₂—N(Rₘ)(Rₙ)—, where each Rₗ, Rₘ and Rₙ is, independently, H or substituted or unsubstituted C₁-C₁₀ alkyl.

Additional examples of modified sugar moieties include a 2′-OMe modified sugar moiety, bicyclic sugar moiety, 2′-O-(2-methoxyethyl) (2′-MOE), 2′-deoxy-2′-fluoro nucleoside, 2′-fluoro-β-D-arabinonucleoside, locked nucleic acid (LNA), constrained ethyl 2′-4′-bridged nucleic acid (cEt), S-cEt, tcDNA, hexitol nucleic acids (HNA), and tricyclic analog (e.g., tcDNA).

As used herein, “bicyclic nucleosides” refer to modified nucleosides comprising a bicyclic sugar moiety. Examples of bicyclic nucleosides include without limitation nucleosides comprising a bridge between the 4′ and the 2′ ribosyl ring atoms. In certain embodiments, antisense compounds provided herein include one or more bicyclic nucleosides comprising a 4′ to 2′ bridge. Examples of such 4′ to 2′ bridged bicyclic nucleosides, include but are not limited to one of the formulae: 4′-(CH₂)—O-2′ (LNA); 4′-(CH₂)—S-2′; 4′-(CH₂)₂—O-2′ (ENA); 4′-CH(CH₃)—O-2′ and 4′-CH(CH₂OCH₃)—O-2′ (and analogs thereof see U.S. Pat. No. 7,399,845, issued on Jul. 15, 2008); 4′-C(CH₃)(CH₃)—O-2′ (and analogs thereof see published International Application WO/2009/006478, published Jan. 8, 2009); 4′-CH₂—N(OCH₃)-2′ (and analogs thereof see published International Application WO/2008/150729, published Dec. 11, 2008); 4′-CH₂—O—N(CH₃)-2′ (see published U.S. Patent Application US2004-0171570, published Sep. 2, 2004); 4′-CH₂—N(R)—O-2′, wherein R is H, C₁-C₁₂ alkyl, or a protecting group (see U.S. Pat. No. 7,427,672, issued on Sep. 23, 2008); 4′-CH₂—C(H)(CH₃)-2′ (see Chattopadhyaya et al., J. Org. Chem., 2009, 74, 118-134); and 4′-CH₂—C—(═CH₂)-2′ (and analogs thereof see published International Application WO 2008/154401, published on Dec. 8, 2008).

Further reports related to bicyclic nucleosides can also be found in published literature (see for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129(26) 8362-8379; Elayadi et al., Curr. Opinion Invest. Drugs, 2001, 2, 558-561; Braasch et al., Chem. Biol., 2001, 8, 1-7; and Orum et al., Curr. Opinion Mol. Ther., 2001, 3, 239-243; U.S. Pat. Nos. 6,268,490; 6,525,191; 6,670,461; 6,770,748; 6,794,499; 7,034,133; 7,053,207; 7,399,845; 7,547,684; and 7,696,345; U.S. Patent Publication No. US2008-0039618; US2009-0012281; U.S. Patent Ser. No. 60/989,574; 61/026,995; 61/026,998; 61/056,564; 61/086,231; 61/097,787; and 61/099,844; Published PCT International applications WO 1994/014226; WO 2004/106356; WO 2005/021570; WO 2007/134181; WO 2008/150729; WO 2008/154401; and WO 2009/006478. Each of the foregoing bicyclic nucleosides can be prepared having one or more stereochemical sugar configurations including for example α-L-ribofuranose and β-D-ribofuranose (see PCT international application PCT/DK98/00393, published on Mar. 25, 1999 as WO 99/14226).

In certain embodiments, bicyclic sugar moieties of BNA nucleosides include, but are not limited to, compounds having at least one bridge between the 4′ and the 2′ position of the pentofuranosyl sugar moiety wherein such bridges independently comprises 1 or from 2 to 4 linked groups independently selected from —[C(Rₐ)(R_{b})]ₙ—, —C(Rₐ)═C(R_{b})—, —C(Rₐ)═N—, —C(═O)—, —C(═NRₐ)—, —C(═S)—, —O—, —Si(Rₐ)₂—, —S(═O)ₓ—, and —N(Rₐ)—;
wherein:
x is 0, 1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

In certain embodiments, the bridge of a bicyclic sugar moiety is —[C(Rₐ)(R_{b})]ₙ—, —[—[C(Rₐ)(R_{b})]ₙ—O—, —C(Rₐ)(R_{b})—N(R)—O— or —C(RₐR_{b})—O—N(R)—. In certain embodiments, the bridge is 4′-CH₂-2′, 4′-(CH₂)₂-2′, 4′—(CH₂)₃-2′, 4′—CH₂—O-2′, 4′—(CH₂)₂—O-2′, 4′—CH₂—O—N(R)-2′ and 4′-CH₂—N(R)—O-2′- wherein each R is, independently, H, a protecting group or C₁-C₁₂ alkyl, each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group; and R is H, C₁-C₁₂ alkyl, or a protecting group (see U.S. Pat. No. 7,427,672, issued on Sep. 23, 2008).

In certain embodiments, bicyclic nucleosides are further defined by isomeric configuration. For example, a nucleoside comprising a 4′-2′ methylene-oxy bridge, may be in the α-L configuration or in the β-D configuration. Previously, α-L-methyleneoxy (4′-CH₂—O-2′) BNA's have been incorporated into antisense oligonucleotides that showed antisense activity (Frieden et al., Nucleic Acids Research, 2003, 21, 6365-6372).

In certain embodiments, bicyclic nucleosides include, but are not limited to, α-L-methyleneoxy (4′-CH₂—O-2′) BNA, β-D-methyleneoxy (4′-CH₂—O-2′) BNA, ethyleneoxy (4′-(CH₂)₂—O-2) BNA, aminooxy (4′-CH₂—O—N(R)-2′) BNA, oxyamino (4′-CH₂—N(R)—O-2′) BNA, methyl(methyleneoxy) (4′-CH(CH₃)—O-2′) BNA, methylene-thio (4′-CH₂—S-2′) BNA, methylene-amino (4′-CH₂—N(R)-2′) BNA, methyl carbocyclic (4′-CH₂—CH(CH₃)-2′) BNA, and propylene carbocyclic (4′-(CH₂)₃-2′) BNA; wherein R is H, C₁-C₁₂ alkyl, or a protecting group (see U.S. Pat. No. 7,427,672, issued on Sep. 23, 2008).

The present disclosure provides, in some embodiments, methods for treating, ameliorating, or preventing a neurological disease and/or a neuropathy further include methods of administering, to a patient, a pharmaceutically acceptable composition, for example, a pharmaceutically acceptable formulation that includes one or more STMN2 oligonucleotides, KCNQ2 oligonucleotides, UNC13A oligonucleotides, and/or SMN2 oligonucleotides. STMN2 oligonucleotides can increase, restore, or stabilize STMN2 activity, for example, STMN2 activity, and/or levels of STMN2 expression, for example, STMN2 mRNA and/or protein expression. KCNQ2 oligonucleotides can increase, restore, or stabilize KCNQ2 activity, for example, KCNQ2 activity, and/or levels of KCNQ2 expression, for example, KCNQ2 mRNA and/or protein expression. UNC13A oligonucleotides can increase, restore, or stabilize UNC13A activity, for example, UNC13A activity, and/or levels of UNC13A expression, for example, UNC13A mRNA and/or protein expression. SMN2 oligonucleotides can increase, restore, or stabilize SMN2 activity, for example, SMN2 activity, and/or levels of SMN2 expression, for example, SMN2 mRNA and/or protein expression.

The present disclosure also provides pharmaceutical compositions comprising a STMN2 oligonucleotide, KCNQ2 oligonucleotide, UNC13A oligonucleotide, and/or SMN2 oligonucleotide formulated together with one or more pharmaceutically or cosmetically acceptable excipients. These formulations include those suitable for oral, sublingual, intratracheal, intranasal, transdermal, pulmonary, intrathecal, intrathalamic, intracisternal, intracerebroventricular, parenteral (e.g., subcutaneous, intramuscular, intradermal, intraduodenal, or intravenous) administration, transmucosal (e.g., buccal, vaginal, and rectal), or for topical use, e.g., as part of a composition suitable for applying topically to skin and/or mucous membrane, for example, a composition in the form of a gel, a paste, a wax, a cream, a spray, a liquid, a foam, a lotion, an ointment, a topical solution, a transdermal patch, a powder, a vapor, or a tincture. Although the most suitable form of administration in any given case will depend on the degree and severity of the condition being treated and on the nature of the particular STMN2 oligonucleotide, KCNQ2 oligonucleotide, UNC13A oligonucleotide, and/or SMN2 oligonucleotide being used.

The present disclosure also provides a pharmaceutical composition comprising a STMN2 oligonucleotide or a pharmaceutically acceptable salt thereof (for example, a STMN2 AON that includes a sequence of any of SEQ ID NOs: 1-466, SEQ ID NO: 893-1338, SEQ ID NOs: 1342-1366, SEQ ID NOs: 1392-1664, and SEQ ID NOs: 10655-10669). The present disclosure also provides a pharmaceutical composition comprising a KCNQ2 oligonucleotide or a pharmaceutically acceptable salt thereof (for example, a KCNQ2 AON that includes a sequence of any of SEQ ID NOs: 1676-1851, SEQ ID NOs: 2028-2529, SEQ ID NOs: 3046-3221, SEQ ID NOs: 3398-3899, and SEQ ID NOs 4402-4530). The present disclosure also provides a pharmaceutical composition comprising a UNC13A oligonucleotide or a pharmaceutically acceptable salt thereof (for example, a UNC13A AON that includes a sequence of any of SEQ ID NOs: 4531-5794, SEQ ID NO: 7059-8322, SEQ ID NOs: 9596-9696, or SEQ ID NOs: 10670-10779). The present disclosure also provides a pharmaceutical composition comprising a SMN2 oligonucleotide or a pharmaceutically acceptable salt thereof (for example, a SMN2 AON that includes a sequence of any of SEQ ID NOs: 9710-10141 and SEQ ID NOs: 10574-10581).

The present disclosure also provides methods that include the use of pharmaceutical compositions comprising a STMN2 AON, KCNQ2 AON, UNC13A AON, and/or SMN2 AON is formulated together with one or more pharmaceutically acceptable excipients. Exemplary compositions provided herein include compositions comprising a STMN2 AON, KCNQ2 AON, UNC13A AON, and/or SMN2 AON, and one or more pharmaceutically acceptable excipients. Formulations include those suitable for oral, sublingual, intratracheal, intranasal, transdermal, pulmonary, intrathecal, intrathalamic, intracisternal, intracerebroventricular, parenteral (e.g., subcutaneous, intramuscular, intradermal, intraduodenal, or intravenous) administration, transmucosal (e.g., buccal, vaginal, and rectal), or for topical use. The most suitable form of administration in any given case will depend on the clinical symptoms, complications, or biochemical indicia of the state, disorder, disease, or condition that one is trying to prevent in a subject; the state, disorder, disease, or condition one is trying to prevent in a subject; and/or on the nature of the particular compound and/or the composition being used.

### Additional Chemically Modified Oligonucleotides

STMN2 AONs, KCNQ2 AONs, UNC13A AONs, and/or SMN2 AONs described herein, can include chemically modified nucleosides, including modified ribonucleosides and modified deoxyribonucleosides. Chemically modified nucleosides include, but are not limited to, uracine, uridine, 2′-O-(2-methoxyethyl) modifications, for example, 2′-O-(2-methoxyethyl)guanosine, 2′-O-(2-methoxyethyl)adenosine, 2′-O-(2-methoxyethyl)cytosine, and 2′-O-(2-methoxyethyl)thymidine. In certain embodiments, mixed modalities, e.g., a combination of a STMN2 peptide nucleic acid (PNA) and a STMN2 locked nucleic acid (LNA), a combination of a KCNQ2 peptide nucleic acid (PNA) and a KCNQ2 locked nucleic acid (LNA), a combination of a UNC13A peptide nucleic acid (PNA) and a UNC13A locked nucleic acid (LNA), and/or a combination of a SMN2 peptide nucleic acid (PNA) and a SMN2 locked nucleic acid (LNA). Chemically modified nucleosides also include, but are not limited to, locked nucleic acids (LNAs), 2′-O-methyl, 2′-fluoro, and 2′-fluoro-β-D-arabinonucleotide (FANA), and Fluoro Cyclohexenyl nucleic acid (F-CeNA) modifications. Chemically modified nucleosides that can be included in STMN2 AONs described herein are described in Johannes and Lucchino, (2018) “Current Challenges in Delivery and Cytosolic Translocation of Therapeutic RNAs” Nucleic Acid Ther. 28(3): 178-93; Rettig and Behlke, (2012) “Progress toward in vivo use of siRNAs-II” Mol Ther 20:483-512; and Khvorova and Watts, (2017) “The chemical evolution of oligonucleotide therapies of clinical utility” Nat Biotechnol., 35(3):238-48, the contents of each of which are incorporated by reference herein.

STMN2 AONs, KCNQ2 AONs, UNC13A AONs, and/or SMN2 AONs described herein can include chemical modifications that promote stabilization of an oligonucleotide's terminal 5′-phosphate and phosphatase-resistant analogs of 5′-phosphate. Chemical modifications that promote oligonucleotide terminal 5′-phosphate stabilization or which are phosphatase-resistant analogs of 5′-phosphate include, but are not limited to, 5′-methyl phosphonate, 5′-methylenephosphonate, 5′-methylenephosphonate analogs, 5′-E-vinyl phosphonate (5′-E-VP), 5′-phosphorothioate, and 5′-C-methyl analogs. Chemical modifications that promote AON terminal 5′-phosphate stabilization and phosphatase-resistant analogues of 5′-phosphate are described in Khvorova and Watts, (2017) “The chemical evolution of oligonucleotide therapies of clinical utility” Nat Biotechnol., 35(3):238-48, the contents of which are incorporated by reference herein.

In some embodiments described herein, STMN2 AONs, KCNQ2 AONs, UNC13A AONs, and/or SMN2 AONs described herein can include chemically modified nucleosides, for example, 2′ O-methyl ribonucleosides, for example, 2′ O-methyl cytidine, 2′ O-methyl guanosine, 2′ O-methyl uridine, and/or 2′ O-methyl adenosine. STMN2 AONs, KCNQ2 AONs, UNC13A AONs, and/or SMN2 AONs described herein can include one or more chemically modified bases, including a 5-methylpyrimidine, for example, 5-methylcytosine, and/or a 5-methylpurine, for example, 5-methylguanine. Chemically modified nucleosides can further include pseudo-uridine or 5′methoxyuridine. STMN2 AONs, KCNQ2 AONs, UNC13A AONs, and/or SMN2 AONs described herein can include any of the following chemically modified nucleosides: 5-methyl-2′-O-methylcytidine, 5-methyl-2′-O-methylthymidine, 5-methylcytidine, 5-methyluridine, and/or 5-methyl 2′-deoxycytidine.

STMN2 AONs, KCNQ2 AONs, UNC13A AONs, and/or SMN2 AONs described herein can include a phosphate backbone where one or more of the oligonucleoside linkages is a phosphate linkage. STMN2 AONs, KCNQ2 AONs, UNC13A AONs, and/or SMN2 AONs described herein may include a modified oligonucleotide backbone, where one or more of the nucleoside linkages of the sequence is selected from the group consisting of a phosphorothioate linkage, a phosphorodithioate linkage, a phosphotriester linkage, an alkylphosphonate linkage, a 3-methoxypropyl phosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage. In some embodiments of STMN2 AONs, KCNQ2 AONs, UNC13A AONs, and/or SMN2 AONs described herein, at least one (i.e., one or more) internucleoside linkage of the oligonucleotide is a phosphorothioate linkage. For example, in some embodiments of STMN2 AONs, KCNQ2 AONs, UNC13A AONs, and/or SMN2 AONs described herein, one, two, three, or more internucleoside linkages of the oligonucleotide is a phosphorothioate linkage. In preferred embodiments of STMN2 AONs, KCNQ2 AONs, UNC13A AONs, and/or SMN2 AONs described herein, all internucleoside linkages of the oligonucleotide are phosphorothioate linkages.

### STMN2

In some embodiments, all of the nucleotide linkages of a STMN2 AON of any of SEQ ID NOs: 1-466, SEQ ID NO: 893-1338, SEQ ID NOs: 1342-1366, SEQ ID NOs: 1392-1664, and SEQ ID NOs: 10655-10669 are phosphorothioate linkages. In some embodiments, one or more of the nucleotide linkages of a STMN2 AON of any of SEQ ID NOs: 1-466, SEQ ID NO: 893-1338, SEQ ID NOs: 1342-1366, SEQ ID NOs: 1392-1664, and SEQ ID NOs: 10655-10669 are phosphorothioate linkages.

In various embodiments, nucleotide linkages of STMN2 AON described herein such as any of SEQ ID NOs: 1-466, SEQ ID NO: 893-1338, SEQ ID NOs: 1342-1366, SEQ ID NOs: 1392-1664, and SEQ ID NOs: 10655-10669 include a mix of phosphodiester and phosphorothioate linkages.

In some embodiments, nucleoside linkages linking a base at position 3 of a STMN2 AON described herein are phosphodiester bonds. For example, the base at position 3 may be linked to each adjacent base (e.g., preceding base and succeeding base) through a phosphodiester bond. An example 25mer STMN2 AON with phosphodiester bonds linking the base at position 3 can be denoted as:

XXoDoXXXXXXXXXXXXXXXXXXXXXX

where “o” represents a phosphodiester bond and “D” represents the base at position 3. Any nucleobase in the AON can be a nucleobase analog.

In some embodiments, one of the nucleoside linkages linking a base at position 3 of a STMN2 AON described herein is a phosphodiester bond. For example, the base at position 3 may be linked to either the preceding base or the succeeding base through a phosphodiester bond. An example 25mer STMN2 AON with a phosphodiester bond linking the base at position 3 to a preceding base can be denoted as:

XXoDXXXXXXXXXXXXXXXXXXXXXX

where “o” represents a phosphodiester bond and “D” represents the base at position 3. Any nucleobase in the AON can be a nucleobase analog.

An example 25mer STMN2 AON with a phosphodiester bond linking the base at position 3 to a succeeding base can be denoted as:

XXDoXXXXXXXXXXXXXXXXXXXXXX

where “o” represents a phosphodiester bond and “D” represents the base at position 3. Any nucleobase in the AON can be a nucleobase analog.

In various embodiments, in addition to one of the nucleoside linkages linking a base at position 3 of a STMN2 AON described herein being a phosphodiester bond, the STMN2 AON further includes two spacers. The two spacers can be positioned in the STMN2 AON such that the STMN2 AON includes a segment with at most 7 linked nucleosides. An example 25mer STMN2 AON with two spacers and with a phosphodiester bond linking the base at position 3 to a preceding base can be denoted as:

XxoDS1XXXXXXXXXS2XXXXXXXXXXX

where “S₁” represents a first spacer, “S₂” represents a second spacer, “o” represents a phosphodiester bond and “D” represents the base at position 3. Any nucleobase in the AON can be a nucleobase analog.

An example 25mer STMN2 AON with two spacers and with a phosphodiester bond linking the base at position 3 to a succeeding base can be denoted as:

XXDoXXXXXXXS1XXXXXXXXXS2XXXX

where “S₁” represents a first spacer, “S₂” represents a second spacer, “o” represents a phosphodiester bond and “D” represents the base at position 3. Any nucleobase in the AON can be a nucleobase analog.

In some embodiments, nucleoside linkages linking a base at position 4 of a STMN2 AON described herein are phosphodiester bonds. For example, the base at position 4 may be linked to each adjacent base (e.g., preceding base and succeeding base) through a phosphodiester bond. An example 25mer STMN2 AON with phosphodiester bonds linking the base at position 4 can be denoted as:

XXXoDoXXXXXXXXXXXXXXXXXXXXX

where “o” represents a phosphodiester bond an “D” represents the base at position 4. Any nucleobase in the AON can be a nucleobase analog.

In some embodiments, one of the nucleoside linkages linking a base at position 4 of a STMN2 AON described herein is a phosphodiester bond. For example, the base at position 4 may be linked to either the preceding base or the succeeding base through a phosphodiester bond. An example 25mer STMN2 AON with a phosphodiester bond linking the base at position 4 to a preceding base can be denoted as:

XXXoDXXXXXXXXXXXXXXXXXXXXX

where “o” represents a phosphodiester bond and “D” represents the base at position 4. Any nucleobase in the AON can be a nucleobase analog.

An example 25mer STMN2 AON with a phosphodiester bond linking the base at position 4 to a succeeding base can be denoted as:

XXXDoXXXXXXXXXXXXXXXXXXXXX

where “o” represents a phosphodiester bond and “D” represents the base at position 4. Any nucleobase in the AON can be a nucleobase analog.

In some embodiments, nucleoside linkages linking both bases at position 3 and position 4 of a STMN2 AON described herein are phosphodiester bonds. For example, the base at position 3 may be linked to each adjacent base (e.g., preceding base and succeeding base) through a phosphodiester bond, and the base at position 4 may be linked to each adjacent base (e.g., preceding base and succeeding base) through a phosphodiester bond. An example 25mer STMN2 AON with phosphodiester bonds linking the bases at positions 3 and 4 can be denoted as:

XXoDoEoXXXXXXXXXXXXXXXXXXXXX

where “o” represents a phosphodiester bond, “D” represents the base at position 3, and “E” represents the base at position 4. In various embodiments, all other bases of the STMN2 AON are linked through phosphorothioate bonds. Any nucleobase in the AON can be a nucleobase analog.

In various embodiments, STMN2 AON described herein include one or more spacers and phosphodiester bonds are located relative to the one or more spacers. In some embodiments, the Y number of bases immediately preceding a spacer are linked through phosphodiester bonds. In various embodiments, Y is one, two, three, four, five, six, seven, eight, nine, ten, eleven, or twelve bases. In particular embodiments, Y is two bases. For example, if the spacer is located at position 15, the bases at positions 13 and 14 of the STMN2 AON are each linked to their respective adjacent bases through phosphodiester bonds. As described herein, the spacer can be located at various positions in the STMN2 AON and therefore, the 2 bases immediately preceding the spacer can vary within the STMN2 AON depending on where the spacer is situated.

In various embodiments, the STMN2 AON may include more than one spacer. In some embodiments, only one of the spacers has Y number of bases immediately preceding the spacer that are linked through phosphodiester bonds. In such embodiments, the other spacers are linked to respective preceding bases through phosphorothioate bonds. In various embodiments, two of the spacers have Y number of bases immediately preceding the spacers that are linked through phosphodiester bonds. In various embodiments, each of the spacers in the STMN2 AON have Y number of bases immediately preceding the spacers that are linked through phosphodiester bonds. In various embodiments, all other bases of the STMN2 AON are linked through phosphorothioate bonds.

In some embodiments, Y number of bases immediately preceding a spacer and Z number of bases immediately succeeding a spacer are linked through phosphodiester bonds. In various embodiments, Y is one, two, three, four, five, six, seven, eight, nine, ten, eleven, or twelve bases. In various embodiments, Z is one, two, three, four, five, six, seven, eight, nine, ten, eleven, or twelve bases. Y and Z can be independent of each other. In particular embodiments, Y is one base and Z is one base. For example, if the spacer is located at position 15, the bases at positions 14 and 16 of the STMN2 AON are each linked to their respective adjacent bases through phosphodiester bonds. To provide an example, such a STMN2 AON (e.g., 25mer) can be denoted as:

XXXXXXXXXXXXXoDoSoEoXXXXXXXXX

where “S” represents a spacer, “o” represents a phosphodiester bond, “D” represents a base immediately preceding the spacer, and “E” represents the base immediately succeeding the spacer. Any nucleobase in the AON can be a nucleobase analog.

As described herein, the spacer can be located at various positions in the STMN2 AON and therefore, the bases immediately preceding or immediately succeeding the spacer can vary within the STMN2 AON depending on where the spacer is situated.

In various embodiments, the STMN2 AON may include more than one spacer. In some embodiments, only one of the spacers has Y number of bases immediately preceding the spacer and Z number of bases immediately succeeding the spacer that are linked through phosphodiester bonds. In such embodiments, the other spacers of the STMN2 AON are linked to respective preceding and succeeding bases through phosphorothioate bonds. To provide an example, such a STMN2 AON (e.g., 25mer) can be denoted as:

XXXXoDoS₁oEoXXXXXXXXXXXS₂XXXXXX

where “S₁” represents a first spacer, “S₂” represents a second spacer, “o” represents a phosphodiester bond, “D” represents a base immediately preceding the spacer, and “E” represents the base immediately succeeding the spacer. Any nucleobase in the AON can be a nucleobase analog.

As another example, such a STMN2 AON (e.g., 25mer) can be denoted as:

XXXXXS₁XXXXXXXXXXXoDoS₂oDoXXXXX

where “S₁” represents a first spacer, “S₂” represents a second spacer, “o” represents a phosphodiester bond, “D” represents a base immediately preceding the spacer, and “E” represents the base immediately succeeding the spacer. Any nucleobase in the AON can be a nucleobase analog.

In some embodiments, one of the spacers is linked to the immediately preceding base through a phosphodiester bond. For example, a STMN2 AON includes a first spacer that is linked to the immediately preceding base through a phosphodiester bond, which can be denoted as:

XXXXXXoS₁XXXXXXXXXXXS₂XXXXXX

where “S₁” represents a first spacer, “S₂” represents a second spacer, “o” represents a phosphodiester bond. Any nucleobase in the AON can be a nucleobase analog.

As another example, a STMN2 AON includes a second spacer that is linked to the immediately preceding base through a phosphodiester bond, which can be denoted as:

XXXXXXS₁XXXXXXXXXXXoS₂XXXXXX

where “S₁” represents a first spacer, “S₂” represents a second spacer, “o” rea phosphodiester bond. Any nucleobase in the AON can be a nucleobase analog.

In various embodiments, the STMN2 AON may be a AON variant (e.g., a 23mer, a 21mer, or a 19mer) where one of the spacers is linked to the immediately preceding base through a phosphodiester bond. For example, the STMN2 AON may be a 21mer with a first spacer that is linked to the immediately preceding base through a phosphodiester bond, which can be denoted as:

XXXXXXXoS₁XXXXXS₂XXXXXXX

where “S₁” represents a first spacer, “S₂” represents a second spacer, “o” represents a phosphodiester bond. Any nucleobase in the AON can be a nucleobase analog.

As another example, the STMN2 AON may be a 21mer with a second spacer that is linked to the immediately preceding base through a phosphodiester bond, which can be denoted as:

XXXXXXXS₁XXXXXoS₂XXXXXXX

where “S₁” represents a first spacer, “S₂” represents a second spacer, “o” represents a phosphodiester bond. Any nucleobase in the AON can be a nucleobase analog.

In some embodiments, the STMN2 AON may be a AON variant (e.g., a 23mer, a 21mer, or a 19mer) where one of the spacers is linked to the immediately preceding base through a phosphodiester bond and the immediately preceding base is further linked to the preceding base through a phosphodiester bond. An example 21mer STMN2 AON can be denoted as:

XXXEoDoS₁XXXXXXS₂XXXXXXX

where “S₁” represents a first spacer, “S₂” represents a second spacer, “o” represents a phosphodiester bond, “D” represents the base immediately preceding S₁ and “E” represents the base immediately preceding “D.” Any nucleobase in the AON can be a nucleobase analog.

As another example, a 21mer STMN2 AON can be denoted as:

XXXXXS₁XXXXEoDoS₂XXXXXXX

where “S₁” represents a first spacer, “S₂” represents a second spacer, “o” represents a phosphodiester bond, “D” represents the base immediately preceding S₂ and “E” represents the base immediately preceding “D.” Any nucleobase in the AON can be a nucleobase analog.

In some embodiments, the STMN2 AON may be a AON variant (e.g., a 23mer, a 21mer, or a 19mer) where a base that immediately precedes a first spacer is linked to another base through a phosphodiester bond. The base that immediately precedes the first spacer may be linked to the first spacer through a non-phosphodiester bond, such as a phosphorothioate bond. Additionally a second spacer is linked to an immediately preceding base through a phosphodiester bond. An example of a 21mer STMN2 AON can be denoted as:

XXXEoDS₁XXXXXXoS₂XXXXXXX

where “S₁” represents a first spacer, “S₂” represents a second spacer, “o” represents a phosphodiester bond, “D” represents the base immediately preceding S₁ and “E” represents the base immediately preceding “D.” Here, the base “D” is linked to the first spacer S₁ through a non-phosphodiester bond (e.g., phosphorothioate bond). Additionally, the base “D” is linked to base “E” through a phosphodiester bond. The second spacer S₂ is linked to an immediately preceding base through a phosphodiester bond. Any nucleobase in the AON can be a nucleobase analog.

Another example of such a 21mer STMN2 AON can be denoted as:

XXXXXoS₁XXXXEoDS₂XXXXXXX

where “S₁” represents a first spacer, “S₂” represents a second spacer, “o” represents a phosphodiester bond, “D” represents the base immediately preceding S₂ and “E” represents the base immediately preceding “D.” Here, the base “D” is linked to the second spacer S₂ through a non-phosphodiester bond (e.g., phosphorothioate bond). Additionally, the base “D” is linked to base “E” through a phosphodiester bond. The first spacer Si is linked to an immediately preceding base through a phosphodiester bond. Any nucleobase in the AON can be a nucleobase analog.

In some embodiments, one of the spacers is linked to the immediately succeeding base through a phosphodiester bond. For example, a STMN2 AON includes a first spacer that is linked to the immediately succeeding base through a phosphodiester bond, which can be denoted as:

XXXXXXS₁oXXXXXXXXXXXS₂XXXXXX

where “S₁” represents a first spacer, “S₂” represents a second spacer, “o” represents a phosphodiester bond. Any nucleobase in the AON can be a nucleobase analog.

As another example, a STMN2 AON includes a second spacer that is linked to the immediately succeeding base through a phosphodiester bond, which can be denoted as:

XXXXXXS₁XXXXXXXXXXXS₂oXXXXXX

where “S₁” represents a first spacer, “S₂” represents a second spacer, “o” represents a phosphodiester bond. Any nucleobase in the AON can be a nucleobase analog.

In various embodiments, the STMN2 AON may be a AON variant (e.g., a 23mer, a 21mer, or a 19mer) where one of the spacers is linked to the immediately succeeding base through a phosphodiester bond. For example, the STMN2 AON may be a 21mer with a first spacer that is linked to the immediately succeeding base through a phosphodiester bond, which can be denoted as:

XXXXXXXS₁oXXXXXS₂XXXXXXX

where “S₁” represents a first spacer, “S₂” represents a second spacer, “o” represents a phosphodiester bond. Any nucleobase in the AON can be a nucleobase analog.

As another example, the STMN2 AON may be a 21mer with a second spacer that is linked to the immediately succeeding base through a phosphodiester bond, which can be denoted as:

XXXXXXXS₁XXXXXS₂oXXXXXXX

where “S₁” represents a first spacer, “S₂” represents a second spacer, “o” represents a phosphodiester bond. Any nucleobase in the AON can be a nucleobase analog.

In various embodiments, two of the spacers have Y number of bases immediately preceding the spacers and Z number of bases immediately succeeding the spacers that are linked through phosphodiester bonds. In various embodiments, each of the spacers in the STMN2 AON have Y number of bases immediately preceding the spacers and Z number of bases immediately succeeding the spacers that are linked through phosphodiester bonds. An example of such a STMN2 AON (e.g., 25mer) can be denoted as:

XXXXoDoS₁oEoXXXXXXXXXXoFOS₂OHoXXXXX

where “S₁” represents a first spacer, “S₂” represents a second spacer, “o” represents a phosphodiester bond, “D” represents a base immediately preceding the first spacer, “E” represents the base immediately succeeding the first spacer, “F” represents a base immediately preceding the second spacer, and “H” represents the base immediately succeeding the second spacer. In various embodiments, all other bases of the STMN2 AON are linked through phosphorothioate bonds. Any nucleobase in the AON can be a nucleobase analog.

In various STMN2 AON includes two or more spacers and a range of bases located between the two spacers are linked through phosphodiester bonds. In various embodiments, the range of bases include two, three, four, five, six, or seven bases linked through phosphodiester bonds. In particular embodiments, the range of bases include two bases linked through phosphodiester bonds. In particular embodiments, the range of bases include four bases linked through phosphodiester bonds. In various embodiments, all other bases of the STMN2 AON are linked through phosphorothioate bonds. Any nucleobase in the AON can be a nucleobase analog.

In various embodiments, the range of bases linked through phosphodiester bonds are positioned Y number of bases succeeding the first spacer and Z number of preceding the second spacer. In various embodiments, Y is one, two, three, four, five, six, or seven bases. In various embodiments, Z is one, two, three, four, five, six, or seven bases. Y and Z can be independent on each other. Any nucleobase in the AON can be a nucleobase analog.

In particular embodiments, Y is five bases and Z is four bases. To provide an example, such a STMN2 AON (e.g., 25mer) can be denoted as:

XXXXXXXXS₁XXXXoDoEoFoHoXXXS₂XXXX

where “S₁” represents a first spacer, “S₂” represents a second spacer, and “o” represents a phosphodiester bond. The bases “D,” “E,” “F,” and “H” represent the range of bases that are linked through phosphodiester bonds. In this example, the range of bases is located five bases after the first spacer (e.g., D is positioned five bases after the first spacer) and the range of bases is located four bases preceding the second spacer (e.g., H is positioned four bases before the second spacer). Any nucleobase in the AON can be a nucleobase analog.

In particular embodiments, Y is four bases and Z is three bases. To provide an example, such a STMN2 AON (e.g., 23mer) can be denoted as:

XXXXXXXS₁XXXoDoEoXXS₂XXXXXXX

where “S₁” represents a first spacer, “S₂” represents a second spacer, and “o” represents a phosphodiester bond. The bases “D” and “E” represent the range of bases that are linked through phosphodiester bonds. In this example, the range of bases is located four bases after the first spacer (e.g., D is positioned four bases after the first spacer) and the range of bases is located three bases preceding the second spacer (e.g., E is positioned three bases before the second spacer). In various embodiments, the positions of the two spacers differ than shown above and therefore, the range of bases linked through phosphodiester bonds are differently positioned. In various embodiments, all other bases of the STMN2 AON are linked through phosphorothioate bonds. Any nucleobase in the AON can be a nucleobase analog.

Table 25 below further depicts examples of STMN2 AON with a mix of phosphodiester and phosphorothioate linkages. In particular, Table 25 depicts examples of STMN2 AONs including spacers and a mix of phosphodiester and phosphorothioate linkages. Any nucleobase in the AON can be a nucleobase analog.

TABLE 25
Example STMN2 AONs with a mixture of phosphodiester and phosphorothioate
bonds.

| | AON Sequence* (5′ → 3′), where “o” | |
|---|---|---|
| | represents a phosphodiester bond, and | |
| SEQ ID | where “S” indicates a spacer. All other | Bases linked with |
| NO: | linkages are phosphorothioate bonds. | phosphodiester bonds |
| 173 | GAGTCCTGCAATATGAATATAATTT | N/A |
| 1451 | GAoGoSoCCTGCAATATSAATATAATTT | Bases at positions 3 and 4 |
| 1452 | GAoGoToCCTGCASTATGAATATSATTT | Bases at positions 3 and 4 |
| 1453 | GAoGoToCCSGCAATATGAATSTAATTT | Bases at positions 3 and 4 |
| 1454 | GAoGoToCCSGCAATATGAASATAATTT | Bases at positions 3 and 4 |
| 1455 | GToCoCoTGCSATATGAASATAAT | Bases at positions 3 and 4 |
| 1456 | GToCoCoTSCAATATGSATATAAT | Bases at positions 3 and 4 |
| 1457 | GToCoCoTGCSATATGSATATAAT | Bases at positions 3 and 4 |
| 1458 | GAGSCCTGCAAToAoToSAATATAATTT | 2 bases preceding a spacer |
| 1459 | GAGTCCTGCASTATGAAToAoToSATTT | 2 bases preceding a spacer |
| 1460 | GAGTCCSGCAATATGAOAoToSTAATTT | 2 bases preceding a spacer |
| 1461 | GAGTCCSGCAATATGoAoAoSATAATTT | 2 bases preceding a spacer |
| 1462 | GTCCTGCSATATGoAoAoSATAAT | 2 bases preceding a spacer |
| 1463 | GTCCTSCAATAoToGoSATATAAT | 2 bases preceding a spacer |
| 1464 | GTCCTGCSATAoToGoSATATAAT | 2 bases preceding a spacer |
| 1465 | GAoGoSoCoCTGCAATATSAATATAATTT | 1 base preceding and 1 base |
| | | after a spacer |
| 1466 | GAGTCCTGCoAoSoToATGAATATSATTT | 1 base preceding and 1 base |
| | | after a spacer |
| 1467 | GAGTCoCoSoGoCAATATGAATSTAATTT | 1 base preceding and 1 base |
| | | after a spacer |
| 1468 | GAGTCoCoSoGoCAATATGAASATAATTT | 1 base preceding and 1 base |
| | | after a spacer |
| 1469 | GTCCTGoCoSoAoTATGAASATAAT | 1 base preceding and 1 base |
| | | after a spacer |
| 1470 | GTCCoToSoCoAATATGSATATAAT | 1 base preceding and 1 base |
| | | after a spacer |
| 1471 | GTCCTGoCoSoAoTATGSATATAAT | 1 base preceding and 1 base |
| | | after a spacer |
| 1472 | GAGSCCTGCAATAoToSoAoATATAATTT | 1 base preceding and 1 base |
| | | after a spacer |
| 1473 | GAGTCCTGCASTATGAATAoToSoAoTTT | 1 base preceding and 1 base |
| | | after a spacer |
| 1474 | GAGTCCSGCAATATGAAoToSoToAATTT | 1 base preceding and 1 base |
| | | after a spacer |
| 1475 | GAGTCCSGCAATATGAOAoSoAoTAATTT | 1 base preceding and 1 base |
| | | after a spacer |
| 1476 | GTCCTGCSATATGAoAoSoAoTAAT | 1 base preceding and 1 base |
| | | after a spacer |
| 1477 | GTCCTSCAATAToGoSoAoTATAAT | 1 base preceding and 1 base |
| | | after a spacer |
| 1478 | GTCCTGCSATAToGoSoAoTATAAT | 1 base preceding and 1 base |
| | | after a spacer |
| 1479 | GAoGoSoCoCTGCAATAoToSoAoATATAATTT | 1 base preceding AND 1 |
| | | base after EACH spacer |
| 1480 | GAGTCCTGCOAoSOTOATGAATAoToSoAoTTT | 1 base preceding AND 1 |
| | | base after EACH spacer |
| 1481 | GAGTCoCoSoGoCAATATGAAoToSoToAATTT | 1 base preceding AND 1 |
| | | base after EACH spacer |
| 1482 | GAGTCoCoSoGoCAATATGAOAoSoAoTAATTT | 1 base preceding AND 1 |
| | | base after EACH spacer |
| 1483 | GTCCTGoCoSoAoTATGAOAoSoAoTAAT | 1 base preceding AND 1 |
| | | base after EACH spacer |
| 1484 | GTCCoToSOCOAATAToGoSoAoTATAAT | 1 base preceding AND 1 |
| | | base after EACH spacer |
| 1485 | GTCCTGoCoSoAoTAToGoSoAoTATAAT | 1 base preceding AND 1 |
| | | base after EACH spacer |
| 197 | CCTTTCTCTCGAAGGTCTTCTGCCG | N/A |
| 1430 | CCTTTCTCTCGAAGGTCTTCTGC | N/A |
| 1431 | CTTTCTCTCGAAGGTCTTCTGCC | N/A |
| 1486 | CCoToToTCTCTCGAAGGTCTTCTGCCG | Bases at positions 3 and 4 |
| 1487 | CCoToToTCTCSCGAAGGTCTTCSGCCG | Bases at positions 3 and 4 |
| 1488 | CToToToCTCSCGAAGGTSTTCTGCC | Bases at positions 3 and 4 |
| 1489 | TTOToCoTCTSGAAGGTCSTCTGCCG | Bases at positions 3 and 4 |
| 1490 | TTOToCoTCTCGAAGGTCTTCTGCCG | Bases at positions 3 and 4 |
| 1491 | CCoToToTCTCTCGAAGGTCTTCTGC | Bases at positions 3 and 4 |
| 1492 | CCTTTCoToCoSCGAAGGTCTTCSGCCG | 2 bases preceding a spacer |
| 1493 | CTTTCoToCoSCGAAGGTSTTCTGCC | 2 bases preceding a spacer |
| 1494 | TTTCToCoToSGAAGGTCSTCTGCCG | 2 bases preceding a spacer |
| 1495 | CCTTTCTCSCGAAGGTCToToCoSGCCG | 2 bases preceding a spacer |
| 1496 | CTTTCTCSCGAAGOGoToSTTCTGCC | 2 bases preceding a spacer |
| 1497 | TTTCTCTSGAAGGoToCOSTCTGCCG | 2 bases preceding a spacer |
| 1498 | CCTTTCToCoSoCoGAAGGTCTTCSGCCG | 1 base preceding and 1 base |
| | | after a spacer |
| 1499 | CTTTCToCoSoCoGAAGGTSTTCTGCC | 1 base preceding and 1 base |
| | | after a spacer |
| 1500 | TTTCTCoToSoGoAAGGTCSTCTGCCG | 1 base preceding and 1 base |
| | | after a spacer |
| 1501 | CCTTTCTCSCGAAGGTCTToCoSoGoCCG | 1 base preceding and 1 base |
| | | after a spacer |
| 1502 | CTTTCTCSCGAAGGoToSoToTCTGCC | 1 base preceding and 1 base |
| | | after a spacer |
| 1503 | TTTCTCTSGAAGGToCoSoToCTGCCG | 1 base preceding and 1 base |
| | | after a spacer |
| 1504 | CCTTTCToCoSoCoGAAGGTCTToCoSoGoCCG | 1 base preceding AND 1 |
| | | base after EACH spacer |
| 1505 | CTTTCToCoSoCoGAAGGoToSoToTCTGCC | 1 base preceding AND 1 |
| | | base after EACH spacer |
| 1506 | TTTCTCoToSoGoAAGGToCoSoToCTGCCG | 1 base preceding AND 1 |
| | | base after EACH spacer |
| 1507 | CCTTTCTCSCGAAoGoGoToCoTTCSGCCG | Range of 4 bases between |
| | | two spacers |
| 1508 | CTTTCTCSCGAoAoGoGTSTTCTGCC | Range of 2 bases between |
| | | two spacers |
| 1509 | TTTCTCTSGAAoGoGoTCSTCTGCCG | Range of 2 bases between |
| | | two spacers |
| 1510 | GAoGSCCTGCAATATSAATATAATTT | Base 3 linked to preceding |
| | | base through |
| | | phosphodiester linkage |
| 1511 | GAGOTCCTGCASTATGAATATSATTT | Base 3 linked to preceding |
| | | base through |
| | | phosphodiester linkage |
| 1512 | GAGTCCOSGCAATATGAATSTAATTT | First spacer linked to |
| | | preceding base through |
| | | phosphodiester linkage |
| 1513 | GAGTCCSOGCAATATGAASATAATTT | First spacer linked to |
| | | succeeding base through |
| | | phosphodiester linkage |
| 1514 | GTCCTGCSOATATGAASATAAT | First spacer linked to |
| | | succeeding base through |
| | | phosphodiester linkage |
| 1515 | GTCCTGCOSATATGSATATAAT | First spacer linked to |
| | | preceding base through |
| | | phosphodiester linkage |
| 1516 | GTCCoTOSCAATATGSATATAAT | 1 base preceding a first |
| | | spacer linked through |
| | | phosphodiester linkage |
| 1517 | GAGTCCSGCAATATGAAToSTAATTT | Second spacer linked to |
| | | preceding base through |
| | | phosphodiester linkage |
| 1518 | GAGTCCSGCAATATGAASoATAATTT | Second spacer linked to |
| | | succeeding base through |
| | | phosphodiester linkage |
| 1519 | GTCCTGCSATATGAAoSATAAT | Second spacer linked to |
| | | preceding base through |
| | | phosphodiester linkage |
| 1520 | GTCCTGCSATATGSoATATAAT | First spacer linked to |
| | | succeeding base through |
| | | phosphodiester linkage |
| 1521 | GTCCoTSCAATATGOSATATAAT | 1 base preceding a first |
| | | spacer linked through |
| | | phosphodiester linkage and |
| | | second spacer linked to |
| | | preceding base through |
| | | phosphodiester linkage |

In some embodiments, a disclosed STMN2 AON may have at least one modified nucleobase, e.g., 5-methylcytosine, and/or at least one methylphosphonate nucleotide, which is placed, for example, either at only one of the 5′ or 3′ ends or at both 5′ and 3′ ends or along the oligonucleotide sequence.

STMN2 AONs may include at least one modified sugar. For example, the sugar moiety of at least one nucleotide constituting the oligonucleotide is a ribose in which the 2′—OH group may be replaced by any one selected from the group consisting of OR, R, R′OR, SH, SR, NH₂, NR₂, N₃, CN, F, Cl, Br, and I (wherein R is an alkyl or aryl and R′ is an alkylene). Examples of a modified sugar moiety include a 2′-OMe modified sugar moiety, bicyclic sugar moiety, 2′-O-(2-methoxyethyl) (2′-MOE or MOE), 2′-O—(N-methylacetamide), 2′-deoxy-2′-fluoro nucleoside, 2′-fluoro-β-D-arabinonucleoside, locked nucleic acid (LNA), constrained ethyl 2′-4′-bridged nucleic acid (cEt), S-cEt, tcDNA, hexitol nucleic acids (HNA), and tricyclic analog (e.g., tcDNA).

In some embodiments, STMN2 AONs comprise 2′OMe (e.g., a STMN2 AON comprising one or more 2′OMe modified sugar), 2′-MOE or MOE (e.g., a STMN2 AON comprising one or more 2′-MOE modified sugar), PNA (e.g., a STMN2 AON comprising one or more N-(2-aminoethyl)-glycine units linked by amide bonds or carbonyl methylene linkage as repeating units in place of a sugar-phosphate backbone), LNA (e.g., a STMN2 AON comprising one or more locked ribose, and can be a mixture of 2′-deoxy nucleotides or 2′Ome nucleotides), c-ET (e.g., a STMN2 AON comprising one or more cET sugar), cMOE (e.g., a STMN2 AON comprising one or more cMOE sugar), morpholino oligomer (e.g., a STMN2 AON comprising a backbone comprising one or more PMO), deoxy-2′-fluoro nucleoside (e.g., a STMN2 AON comprising one or more 2′-fluoro-β-D-arabinonucleoside), tcDNA (e.g., a STMN2 AON comprising one or more tcDNA modified sugar), ENA (e.g., a STMN2 AON comprising one or more ENA modified sugar), or HNA (e.g., a STMN2 AON comprising one or more HNA modified sugar). In some embodiments, a STMN2 AON comprises one or more phosphorothioate linkage, phosphodiester linkage, phosphotriester linkage, methylphosphonate linkage, phosphoramidate linkage, a phosphoramidothioate linkage, a thiophosphorodiamidate linkage, morpholino linkage, PNA linkage, or any combination of phosphorothioate linkage, phosphodiester linkage, a phosphotriester linkage, methylphosphonate linkage, phosphoramidate linkage, morpholino linkage, and PNA linkage. In some embodiments, a STMN2 AON comprises one or more phosphorothioate linkage, phosphodiester linkage, or a combination of phosphorothioate and phosphodiester linkages.

In some embodiments, STMN2 AONs with a sequence of any one of SEQ ID NOs: 1-466, SEQ ID NO: 893-1338, SEQ ID NOs: 1342-1366, SEQ ID NOs: 1392-1664, and SEQ ID NOs: 10655-10669 is a chirally controlled oligonucleotide, such as a chirally controlled oligonucleotide described in any of U.S. Pat. Nos. 9,982,257, 10,590,413, 10,724,035, 10,450,568, and PCT Publication No. WO2019200185, each of which is hereby incorporated by reference in its entirety.

For example, a STMN2 AON with a sequence of any one of SEQ ID NOs: 1-466, SEQ ID NO: 893-1338, SEQ ID NOs: 1342-1366, SEQ ID NOs: 1392-1664, and SEQ ID NOs: 10655-10669 is a chirally controlled oligonucleotide comprising a plurality of oligonucleotides of at least one type, wherein each type is defined by: 1) base sequence; 2) pattern of backbone linkages; 3) pattern of backbone chiral centers; and 4) pattern of backbone X-moieties (—X-L-R′); wherein: the oligonucleotides of the at least one type comprise one or more phosphorothioate triester internucleotidic linkages and one or more phosphate diester linkage; the oligonucleotides of the at least one type comprise at least two consecutive modified internucleotidic linkages; and oligonucleotides of the at least one oligonucleotide type comprise one or more modified internucleotidic linkages independently having the structure of:

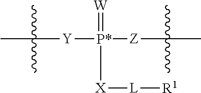

wherein: P* is an asymmetric phosphorus atom and is either Rp or Sp;
W is O, S or Se; each of X, Y and Z is independently —O—, —S—, —N(-L-R¹)—, or L; L is a covalent bond or an optionally substituted, linear or branched C₁-C₅₀ alkylene, wherein one or more methylene units of L are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—; R¹ is halogen, R, or an optionally substituted C₁-C₁₀ aliphatic wherein one or more methylene units are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—; each R′ is independently —R, —C(O)R, —CO₂R, or —SO₂R, or: two R′ on the same nitrogen are taken together with their intervening atoms to form an optionally substituted heterocyclic or heteroaryl ring, or two R′ on the same carbon are taken together with their intervening atoms to form an optionally substituted aryl, carbocyclic, heterocyclic, or heteroaryl ring; -Cy- is an optionally substituted bivalent ring selected from phenylene, carbocyclylene, arylene, heteroarylene, or heterocyclylene; each R is independently hydrogen, or an optionally substituted group selected from C₁-C₆ aliphatic, phenyl, carbocyclyl, aryl, heteroaryl, or heterocyclyl; and each

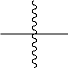

independently represents a connection to a nucleoside. In some embodiments, a STMN2 AON with a sequence of any one of SEQ ID NOs: 1-466, SEQ ID NO: 893-1338, SEQ ID NOs: 1342-1366, SEQ ID NOs: 1392-1664, and SEQ ID NOs: 10655-10669 is a chirally controlled oligonucleotide comprising certain chemical modifications (e.g., 2′F (2′ Fluoro, which contains a fluorine molecule at the 2′ ribose position (instead of 2′-hydroxyl group in an RNA monomer)), 2′-OMe, phosphorothioate linkages, lipid conjugation, etc.), as described in U.S. Pat. No. 10,450,568.

### KCNQ2

In some embodiments, all of the nucleotide linkages of a KCNQ2 AON of any of SEQ ID NOs: 1676-1851, SEQ ID NOs: 2028-2529, SEQ ID NOs: 3046-3221, SEQ ID NOs: 3398-3899, or SEQ ID NOs: 4402-4409 are phosphorothioate linkages. In some embodiments, one or more of the nucleotide linkages of a KCNQ2 AON of any of SEQ ID NOs: 1676-1851, SEQ ID NOs: 2028-2529, SEQ ID NOs: 3046-3221, SEQ ID NOs: 3398-3899, or SEQ ID NOs: 4402-4409 are phosphorothioate linkages.

In various embodiments, nucleotide linkages of KCNQ2 AON described herein such as any of SEQ ID NOs: 1676-1851, SEQ ID NOs: 2028-2529, SEQ ID NOs: 3046-3221, SEQ ID NOs: 3398-3899, or SEQ ID NOs: 4402-4409 include a mix of phosphodiester and phosphorothioate linkages.

In some embodiments, nucleoside linkages linking a base at position 3 of a KCNQ2 AON described herein are phosphodiester bonds. For example, the base at position 3 may be linked to each adjacent base (e.g., preceding base and succeeding base) through a phosphodiester bond. An example 25mer KCNQ2 AON with phosphodiester bonds linking the base at position 3 can be denoted as:

XXoDoXXXXXXXXXXXXXXXXXXXXXX

where “o” represents a phosphodiester bond and “D” represents the base at position 3. Any nucleobase in the AON can be a nucleobase analog.

In some embodiments, one of the nucleoside linkages linking a base at position 3 of a KCNQ2 AON described herein is a phosphodiester bond. For example, the base at position 3 may be linked to either the preceding base or the succeeding base through a phosphodiester bond. An example 25mer KCNQ2 AON with a phosphodiester bond linking the base at position 3 to a preceding base can be denoted as:

XXoDXXXXXXXXXXXXXXXXXXXXXX

where “o” represents a phosphodiester bond and “D” represents the base at position 3. Any nucleobase in the AON can be a nucleobase analog.

An example 25mer KCNQ2 AON with a phosphodiester bond linking the base at position 3 to a succeeding base can be denoted as:

XXDoXXXXXXXXXXXXXXXXXXXXXX

where “o” represents a phosphodiester bond and “D” represents the base at position 3. Any nucleobase in the AON can be a nucleobase analog.

In various embodiments, in addition to one of the nucleoside linkages linking a base at position 3 of a KCNQ2 AON described herein being a phosphodiester bond, the KCNQ2 AON further includes two spacers. The two spacers can be positioned in the KCNQ2 AON such that the KCNQ2 AON includes a segment with at most 7 linked nucleosides. An example 25mer KCNQ2 AON with two spacers and with a phosphodiester bond linking the base at position 3 to a preceding base can be denoted as:

XXoDS₁XXXXXXXXXS₂XXXXXXXXX

where “S₁” represents a first spacer, “S₂” represents a second spacer, “o” represents a phosphodiester bond and “D” represents the base at position 3. Any nucleobase in the AON can be a nucleobase analog.

An example 25mer KCNQ2 AON with two spacers and with a phosphodiester bond linking the base at position 3 to a succeeding base can be denoted as:

XXDoXXXXXXXS₁XXXXXXXXXS₂XXXX

where “S₁” represents a first spacer, “S₂” represents a second spacer, “o” represents a phosphodiester bond and “D” represents the base at position 3. Any nucleobase in the AON can be a nucleobase analog.

In some embodiments, nucleoside linkages linking a base at position 4 of a KCNQ2 AON described herein are phosphodiester bonds. For example, the base at position 4 may be linked to each adjacent base (e.g., preceding base and succeeding base) through a phosphodiester bond. An example 25mer KCNQ2 AON with phosphodiester bonds linking the base at position 4 can be denoted as:

XXXoDoXXXXXXXXXXXXXXXXXXXX

where “o” represents a phosphodiester bond and “D” represents the base at position 4. Any nucleobase in the AON can be a nucleobase analog.

In some embodiments, one of the nucleoside linkages linking a base at position 4 of a KCNQ2 AON described herein is a phosphodiester bond. For example, the base at position 4 may be linked to either the preceding base or the succeeding base through a phosphodiester bond. An example 25mer KCNQ2 AON with a phosphodiester bond linking the base at position 4 to a preceding base can be denoted as:

XXXoDXXXXXXXXXXXXXXXXXXXXX

where “o” represents a phosphodiester bond and “D” represents the base at position 4. Any nucleobase in the AON can be a nucleobase analog.

An example 25mer KCNQ2 AON with a phosphodiester bond linking the base at position 4 to a succeeding base can be denoted as:

XXXDoXXXXXXXXXXXXXXXXXXXXX

where “o” represents a phosphodiester bond and “D” represents the base at position 4. Any nucleobase in the AON can be a nucleobase analog.

In some embodiments, nucleoside linkages linking both bases at position 3 and position 4 of a KCNQ2 AON described herein are phosphodiester bonds. For example, the base at position 3 may be linked to each adjacent base (e.g., preceding base and succeeding base) through a phosphodiester bond, and the base at position 4 may be linked to each adjacent base (e.g., preceding base and succeeding base) through a phosphodiester bond. An example 25mer KCNQ2 AON with phosphodiester bonds linking the bases at positions 3 and 4 can be denoted as:

XXoDoEoXXXXXXXXXXXXXXXXXXXXX

where “o” represents a phosphodiester bond, “D” represents the base at position 3, and “E” represents the base at position 4. In various embodiments, all other bases of the KCNQ2 AON are linked through phosphorothioate bonds. Any nucleobase in the AON can be a nucleobase analog.

In various embodiments, KCNQ2 AON described herein include one or more spacers and phosphodiester bonds are located relative to the one or more spacers. In some embodiments, the Y number of bases immediately preceding a spacer are linked through phosphodiester bonds. In various embodiments, Y is one, two, three, four, five, six, seven, eight, nine, ten, eleven, or twelve bases. In particular embodiments, Y is two bases. For example, if the spacer is located at position 15, the bases at positions 13 and 14 of the KCNQ2 AON are each linked to their respective adjacent bases through phosphodiester bonds. As described herein, the spacer can be located at various positions in the KCNQ2 AON and therefore, the 2 bases immediately preceding the spacer can vary within the KCNQ2 AON depending on where the spacer is situated.

In various embodiments, the KCNQ2 AON may include more than one spacer. In some embodiments, only one of the spacers has Y number of bases immediately preceding the spacer that are linked through phosphodiester bonds. In such embodiments, the other spacers are linked to respective preceding bases through phosphorothioate bonds. In various embodiments, two of the spacers have Y number of bases immediately preceding the spacers that are linked through phosphodiester bonds. In various embodiments, each of the spacers in the KCNQ2 AON have Y number of bases immediately preceding the spacers that are linked through phosphodiester bonds. In various embodiments, all other bases of the KCNQ2 AON are linked through phosphorothioate bonds.

In some embodiments, Y number of bases immediately preceding a spacer and Z number of bases immediately succeeding a spacer are linked through phosphodiester bonds. In various embodiments, Y is one, two, three, four, five, six, seven, eight, nine, ten, eleven, or twelve bases. In various embodiments, Z is one, two, three, four, five, six, seven, eight, nine, ten, eleven, or twelve bases. Y and Z can be independent of each other. In particular embodiments, Y is one base and Z is one base. For example, if the spacer is located at position 15, the bases at positions 14 and 16 of the KCNQ2 AON are each linked to their respective adjacent bases through phosphodiester bonds. To provide an example, such a KCNQ2 AON (e.g., 25mer) can be denoted as:

XXXXXXXXXXXXXoDoSoEoXXXXXXXXX

where “S” represents a spacer, “o” represents a phosphodiester bond, “D” represents a base immediately preceding the spacer, and “E” represents the base immediately succeeding the spacer. Any nucleobase in the AON can be a nucleobase analog.

As described herein, the spacer can be located at various positions in the KCNQ2 AON and therefore, the bases immediately preceding or immediately succeeding the spacer can vary within the KCNQ2 AON depending on where the spacer is situated.

In various embodiments, the KCNQ2 AON may include more than one spacer. In some embodiments, only one of the spacers has Y number of bases immediately preceding the spacer and Z number of bases immediately succeeding the spacer that are linked through phosphodiester bonds. In such embodiments, the other spacers of the KCNQ2 AON are linked to respective preceding and succeeding bases through phosphorothioate bonds. To provide an example, such a KCNQ2 AON (e.g., 25mer) can be denoted as:

XXXXoDoS₁oEoXXXXXXXXXXXS₂XXXXXX

where “S₁” represents a first spacer, “S₂” represents a second spacer, “o” represents a phosphodiester bond, “D” represents a base immediately preceding the spacer, and “E” represents the base immediately succeeding the spacer. Any nucleobase in the AON can be a nucleobase analog.

As another example, such a KCNQ2 AON (e.g., 25mer) can be denoted as:

XXXXXS₁XXXXXXXXXXXoDoS₂oDoXXXXX

where “S₁” represents a first spacer, “S₂” represents a second spacer, “o” represents a phosphodiester bond, “D” represents a base immediately preceding the spacer, and “E” represents the base immediately succeeding the spacer. Any nucleobase in the AON can be a nucleobase analog.

In some embodiments, one of the spacers is linked to the immediately preceding base through a phosphodiester bond. For example, a KCNQ2 AON includes a first spacer that is linked to the immediately preceding base through a phosphodiester bond, which can be denoted as:

XXXXXXoS₁XXXXXXXXXXXS₂XXXXXX

where “S₁” represents a first spacer, “S₂” represents a second spacer, “o” represents a phosphodiester bond. Any nucleobase in the AON can be a nucleobase analog.

As another example, a KCNQ2 AON includes a second spacer that is linked to the immediately preceding base through a phosphodiester bond, which can be denoted as:

XXXXXXS₁XXXXXXXXXXXoS₂XXXXXX

where “S₁” represents a first spacer, “S₂” represents a second spacer, “o” represents a phosphodiester bond. Any nucleobase in the AON can be a nucleobase analog.

In various embodiments, the KCNQ2 AON may be a AON variant (e.g., a 23mer, a 21mer, or a 19mer) where one of the spacers is linked to the immediately preceding base through a phosphodiester bond. For example, the KCNQ2 AON may be a 21mer with a first spacer that is linked to the immediately preceding base through a phosphodiester bond, which can be denoted as:

XXXXXXXoS₁XXXXXS₂XXXXXXX

where “S₁” represents a first spacer, “S₂” represents a second spacer, “o” represents a phosphodiester bond. Any nucleobase in the AON can be a nucleobase analog.

As another example, the KCNQ2 AON may be a 21mer with a second spacer that is linked to the immediately preceding base through a phosphodiester bond, which can be denoted as:

XXXXXXXS₁XXXXXoS₂XXXXXXX

where “S₁” represents a first spacer, “S₂” represents a second spacer, “o” Represents a phosphodiester bond. Any nucleobase in the AON can be a nucleobase analog.

In some embodiments, the KCNQ2 AON may be a AON variant (e.g., a 23mer, a 21mer, or a 19mer) where one of the spacers is linked to the immediately preceding base through a phosphodiester bond and the immediately preceding base is further linked to the preceding base through a phosphodiester bond. An example 21mer KCNQ2 AON can be denoted as:

XXXEoDoS₁XXXXXXS₂XXXXXXX

where “S₁” represents a first spacer, “S₂” represents a second spacer, “o” represents a phosphodiester bond, “D” represents the base immediately preceding S₁ and “E” represents the base immediately preceding “D.” Any nucleobase in the AON can be a nucleobase analog.

As another example, a 21mer KCNQ2 AON can be denoted as:

XXXXXS₁XXXXEoDoS₂XXXXXXX

where “S₁” represents a first spacer, “S₂” represents a second spacer, “o” represents a phosphodiester bond, “D” represents the base immediately preceding S₂ and “E” represents the base immediately preceding “D.” Any nucleobase in the AON can be a nucleobase analog.

In some embodiments, the KCNQ2 AON may be a AON variant (e.g., a 23mer, a 21mer, or a 19mer) where a base that immediately precedes a first spacer is linked to another base through a phosphodiester bond. The base that immediately precedes the first spacer may be linked to the first spacer through a non-phosphodiester bond, such as a phosphorothioate bond. Additionally a second spacer is linked to an immediately preceding base through a phosphodiester bond. An example of a 21mer KCNQ2 AON can be denoted as:

XXXEoDS₁XXXXXXoS₂XXXXXXX

where “S₁” represents a first spacer, “S₂” represents a second spacer, “o” represents a phosphodiester bond, “D” represents the base immediately preceding S₁ and “E” represents the base immediately preceding “D.” Here, the base “D” is linked to the first spacer S₁ through a non-phosphodiester bond (e.g., phosphorothioate bond). Additionally, the base “D” is linked to base “E” through a phosphodiester bond. The second spacer S₂ is linked to an immediately preceding base through a phosphodiester bond. Any nucleobase in the AON can be a nucleobase analog.

Another example of such a 21mer KCNQ2 AON can be denoted as:

XXXXXoS₁XXXXEoDS₂XXXXXXX

where “S₁” represents a first spacer, “S₂” represents a second spacer, “o” represents a phosphodiester bond, “D” represents the base immediately preceding S₂ and “E” represents the base immediately preceding “D.” Here, the base “D” is linked to the second spacer S₂ through a non-phosphodiester bond (e.g., phosphorothioate bond). Additionally, the base “D” is linked to base “E” through a phosphodiester bond. The first spacer Si is linked to an immediately preceding base through a phosphodiester bond. Any nucleobase in the AON can be a nucleobase analog.

In some embodiments, one of the spacers is linked to the immediately succeeding base through a phosphodiester bond. For example, a KCNQ2 AON includes a first spacer that is linked to the immediately succeeding base through a phosphodiester bond, which can be denoted as:

XXXXXXS₁oXXXXXXXXXXXS₂XXXXXX

where “S₁” represents a first spacer, “S₂” represents a second spacer, “o” represents a phosphodiester bond. Any nucleobase in the AON can be a nucleobase analog.

As another example, a KCNQ2 AON includes a second spacer that is linked to the immediately succeeding base through a phosphodiester bond, which can be denoted as:

XXXXXXS₁XXXXXXXXXXXS₂oXXXXXX

where “S₁” represents a first spacer, “S₂” represents a second spacer, “o” represents a phosphodiester bond. Any nucleobase in the AON can be a nucleobase analog.

In various embodiments, the KCNQ2 AON may be a AON variant (e.g., a 23mer, a 21mer, or a 19mer) where one of the spacers is linked to the immediately succeeding base through a phosphodiester bond. For example, the KCNQ2 AON may be a 21mer with a first spacer that is linked to the immediately succeeding base through a phosphodiester bond, which can be denoted as:

XXXXXXXS₁oXXXXXS₂XXXXXXX

where “S₁” represents a first spacer, “S₂” represents a second spacer, “o” represents a phosphodiester bond. Any nucleobase in the AON can be a nucleobase analog.

As another example, the KCNQ2 AON may be a 21mer with a second spacer that is linked to the immediately succeeding base through a phosphodiester bond, which can be denoted as:

XXXXXXXS₁XXXXXS₂oXXXXXXX

where “S₁” represents a first spacer, “S₂” represents a second spacer, “o” represents a phosphodiester bond. Any nucleobase in the AON can be a nucleobase analog.

In various embodiments, two of the spacers have Y number of bases immediately preceding the spacers and Z number of bases immediately succeeding the spacers that are linked through phosphodiester bonds. In various embodiments, each of the spacers in the KCNQ2 AON have Y number of bases immediately preceding the spacers and Z number of bases immediately succeeding the spacers that are linked through phosphodiester bonds. An example of such a KCNQ2 AON (e.g., 25mer) can be denoted as:

XXXXoDoS₁oEoXXXXXXXXXXoFoS₂oHoXXXXX

where “S₁” represents a first spacer, “S₂” represents a second spacer, “o” represents a phosphodiester bond, “D” represents a base immediately preceding the first spacer, “E” represents the base immediately succeeding the first spacer, “F” represents a base immediately preceding the second spacer, and “H” represents the base immediately succeeding the second spacer. In various embodiments, all other bases of the KCNQ2 AON are linked through phosphorothioate bonds. Any nucleobase in the AON can be a nucleobase analog.

In various KCNQ2 AON includes two or more spacers and a range of bases located between the two spacers are linked through phosphodiester bonds. In various embodiments, the range of bases include two, three, four, five, six, or seven bases linked through phosphodiester bonds. In particular embodiments, the range of bases include two bases linked through phosphodiester bonds. In particular embodiments, the range of bases include four bases linked through phosphodiester bonds. In various embodiments, all other bases of the KCNQ2 AON are linked through phosphorothioate bonds. Any nucleobase in the AON can be a nucleobase analog.

In various embodiments, the range of bases linked through phosphodiester bonds are positioned Y number of bases succeeding the first spacer and Z number of preceding the second spacer. In various embodiments, Y is one, two, three, four, five, six, or seven bases. In various embodiments, Z is one, two, three, four, five, six, or seven bases. Y and Z can be independent on each other. Any nucleobase in the AON can be a nucleobase analog.

In particular embodiments, Y is five bases and Z is four bases. To provide an example, such a KCNQ2 AON (e.g., 25mer) can be denoted as:

XXXXXXXXS₁XXXXoDoEoFoHoXXXS₂XXXX

where “S₁” represents a first spacer, “S₂” represents a second spacer, and “o” represents a phosphodiester bond. The bases “D,” “E,” “F,” and “H” represent the range of bases that are linked through phosphodiester bonds. In this example, the range of bases is located five bases after the first spacer (e.g., D is positioned five bases after the first spacer) and the range of bases is located four bases preceding the second spacer (e.g., H is positioned four bases before the second spacer). Any nucleobase in the AON can be a nucleobase analog.

In particular embodiments, Y is four bases and Z is three bases. To provide an example, such a KCNQ2 AON (e.g., 23mer) can be denoted as:

XXXXXXXS₁XXXoDoEoXXS₂XXXXXXX

where “S₁” represents a first spacer, “S₂” represents a second spacer, and “o” represents a phosphodiester bond. The bases “D” and “E” represent the range of bases that are linked through phosphodiester bonds. In this example, the range of bases is located four bases after the first spacer (e.g., D is positioned four bases after the first spacer) and the range of bases is located three bases preceding the second spacer (e.g., E is positioned three bases before the second spacer). In various embodiments, the positions of the two spacers differ than shown above and therefore, the range of bases linked through phosphodiester bonds are differently positioned. In various embodiments, all other bases of the KCNQ2 AON are linked through phosphorothioate bonds. Any nucleobase in the AON can be a nucleobase analog.

In some embodiments, a disclosed KCNQ2 AON may have at least one modified nucleobase, e.g., 5-methylcytosine, and/or at least one methylphosphonate nucleotide, which is placed, for example, either at only one of the 5′ or 3′ ends or at both 5′ and 3′ ends or along the oligonucleotide sequence.

KCNQ2 AONs may include at least one modified sugar. For example, the sugar moiety of at least one nucleotide constituting the oligonucleotide is a ribose in which the 2′—OH group may be replaced by any one selected from the group consisting of OR, R, R′OR, SH, SR, NH₂, NR₂, N₃, CN, F, Cl, Br, and I (wherein R is an alkyl or aryl and R′ is an alkylene). Examples of a modified sugar moiety include a 2′-OMe modified sugar moiety, bicyclic sugar moiety, 2′-O-(2-methoxyethyl) (2′-MOE or MOE), 2′-O—(N-methylacetamide), 2′-deoxy-2′-fluoro nucleoside, 2′-fluoro-β-D-arabinonucleoside, locked nucleic acid (LNA), constrained ethyl 2′-4′-bridged nucleic acid (cEt), S-cEt, tcDNA, hexitol nucleic acids (HNA), and tricyclic analog (e.g., tcDNA).

In some embodiments, KCNQ2 AONs comprise 2′OMe (e.g., a KCNQ2 AON comprising one or more 2′OMe modified sugar), 2′-MOE or MOE (e.g., a KCNQ2 AON comprising one or more 2′-MOE modified sugar), PNA (e.g., a KCNQ2 AON comprising one or more N-(2-aminoethyl)-glycine units linked by amide bonds or carbonyl methylene linkage as repeating units in place of a sugar-phosphate backbone), LNA (e.g., a KCNQ2 AON comprising one or more locked ribose, and can be a mixture of 2′-deoxy nucleotides or 2′OMe nucleotides), c-ET (e.g., a KCNQ2 AON comprising one or more cET sugar), cMOE (e.g., a KCNQ2 AON comprising one or more cMOE sugar), morpholino oligomer (e.g., a KCNQ2 AON comprising a backbone comprising one or more PMO), deoxy-2′-fluoro nucleoside (e.g., a KCNQ2 AON comprising one or more 2′-fluoro-β-D-arabinonucleoside), tcDNA (e.g., a KCNQ2 AON comprising one or more tcDNA modified sugar), ENA (e.g., a KCNQ2 AON comprising one or more ENA modified sugar), or HNA (e.g., a KCNQ2 AON comprising one or more HNA modified sugar). In some embodiments, a KCNQ2 AON comprises one or more phosphorothioate linkage, phosphodiester linkage, phosphotriester linkage, methylphosphonate linkage, phosphoramidate linkage, a phosphoramidothioate linkage, a thiophosphorodiamidate linkage, morpholino linkage, PNA linkage, or any combination of phosphorothioate linkage, phosphodiester linkage, a phosphotriester linkage, methylphosphonate linkage, phosphoramidate linkage, morpholino linkage, and PNA linkage. In some embodiments, a KCNQ2 AON comprises one or more phosphorothioate linkage, phosphodiester linkage, or a combination of phosphorothioate and phosphodiester linkages.

In some embodiments, KCNQ2 AONs with a sequence of any one of SEQ ID NOs: 1676-1851, SEQ ID NOs: 2028-2529, SEQ ID NOs: 3046-3221, SEQ ID NOs: 3398-3899, or SEQ ID NOs: 4402-4409 is a chirally controlled oligonucleotide, such as a chirally controlled oligonucleotide described in any of U.S. Pat. Nos. 9,982,257, 10,590,413, 10,724,035, 10,450,568, and PCT Publication No. WO2019200185, each of which is hereby incorporated by reference in its entirety.

For example, a KCNQ2 AON with a sequence of any one of SEQ ID NOs: 1676-1851, SEQ ID NOs: 2028-2529, SEQ ID NOs: 3046-3221, SEQ ID NOs: 3398-3899, or SEQ ID NOs: 4402-4409 is a chirally controlled oligonucleotide comprising a plurality of oligonucleotides of at least one type, wherein each type is defined by: 1) base sequence, 2) pattern of backbone linkages; 3) pattern of backbone chiral centers; and 4) pattern of backbone X-moieties (—X-L-R¹); wherein: the oligonucleotides of at least one type comprise one or more phosphorothioate triester internucleotidic linkages and one or more phosphate diester linkage; the oligonucleotides of at least one type comprise at least two consecutive modified internucleotidic linkages; and oligonucleotides of at least one oligonucleotide type comprise one or more modified internucleotidic linkages independently having the structure of:

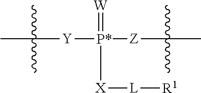

wherein: P* is an asymmetric phosphorus atom and is either Rp or Sp;
W is O, S or Se; each of X, Y and Z is independently —O—, —S—, —N(-L-R¹)—, or L; L is a covalent bond or an optionally substituted, linear or branched C₁-C₅₀ alkylene, wherein one or more methylene units of L are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—; R¹ is halogen, R, or an optionally substituted C₁-C₁₀ aliphatic wherein one or more methylene units are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—; each R′ is independently —R, —C(O)R, —CO₂R, or —SO₂R, or: two R′ on the same nitrogen are taken together with their intervening atoms to form an optionally substituted heterocyclic or heteroaryl ring, or two R′ on the same carbon are taken together with their intervening atoms to form an optionally substituted aryl, carbocyclic, heterocyclic, or heteroaryl ring; -Cy- is an optionally substituted bivalent ring selected from phenylene, carbocyclylene, arylene, heteroarylene, or heterocyclylene; each R is independently hydrogen, or an optionally substituted group selected from C₁-C₆ aliphatic, phenyl, carbocyclyl, aryl, heteroaryl, or heterocyclyl; and each

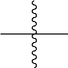

independently represents a connection to a nucleoside. In some embodiments, a KCNQ2 AON with a sequence of any one of SEQ ID NOs: 1676-1851, SEQ ID NOs: 2028-2529, SEQ ID NOs: 3046-3221, SEQ ID NOs: 3398-3899, or SEQ ID NOs: 4402-4409 is a chirally controlled oligonucleotide comprising certain chemical modifications (e.g., 2′F (2′ Fluoro, which contains a fluorine molecule at the 2′ ribose position (instead of 2′-hydroxyl group in an RNA monomer)), 2′-OMe, phosphorothioate linkages, lipid conjugation, etc.), as described in U.S. Pat. No. 10,450,568.

### UNC13A

In some embodiments, all of the nucleotide linkages of a UNC13A AON of any of SEQ ID NOs: 4531-5794, SEQ ID NO: 7059-8322, SEQ ID NOs: 9596-9696, or SEQ ID NOs: 10670-10779 are phosphorothioate linkages. In some embodiments, one or more of the nucleotide linkages of a UNC13A AON of any of SEQ ID NOs: 4531-5794, SEQ ID NO: 7059-8322, SEQ ID NOs: 9596-9696, or SEQ ID NOs: 10670-10779 are phosphorothioate linkages.

In various embodiments, nucleotide linkages of UNC13A AON described herein such as any of SEQ ID NOs: 4531-5794, SEQ ID NO: 7059-8322, SEQ ID NOs: 9596-9696, or SEQ ID NOs: 10670-10779 include a mix of phosphodiester and phosphorothioate linkages.

In some embodiments, nucleoside linkages linking a base at position 3 of a UNC13A AON described herein are phosphodiester bonds. For example, the base at position 3 may be linked to each adjacent base (e.g., preceding base and succeeding base) through a phosphodiester bond. An example 25mer UNC13A AON with phosphodiester bonds linking the base at position 3 can be denoted as:

XXoDoXXXXXXXXXXXXXXXXXXXXXX

where “o” represents a phosphodiester bond and “D” represents the base at position 3. Any nucleobase in the AON can be a nucleobase analog.

In some embodiments, one of the nucleoside linkages linking a base at position 3 of a UNC13A AON described herein is a phosphodiester bond. For example, the base at position 3 may be linked to either the preceding base or the succeeding base through a phosphodiester bond. An example 25mer UNC13A AON with a phosphodiester bond linking the base at position 3 to a preceding base can be denoted as:

XXoDXXXXXXXXXXXXXXXXXXXXXX

where “o” represents a phosphodiester bond and “D” represents the base at position 3. Any nucleobase in the AON can be a nucleobase analog.

An example 25mer UNC13A AON with a phosphodiester bond linking the base at position 3 to a succeeding base can be denoted as:

XXDoXXXXXXXXXXXXXXXXXXXXXX

where “o” represents a phosphodiester bond and “D” represents the base at position 3. Any nucleobase in the AON can be a nucleobase analog.

In various embodiments, in addition to one of the nucleoside linkages linking a base at position 3 of a UNC13A AON described herein being a phosphodiester bond, the UNC13A AON further includes two spacers. The two spacers can be positioned in the UNC13A AON such that the UNC13A AON includes a segment with at most 7 linked nucleosides. An example 25mer UNC13A AON with two spacers and with a phosphodiester bond linking the base at position 3 to a preceding base can be denoted as:

XXoDS₁XXXXXXXXXS₂XXXXXXXXXXX

where “S₁” represents a first spacer, “S₂” represents a second spacer, “o” represents a phosphodiester bond and “D” represents the base at position 3. Any nucleobase in the AON can be a nucleobase analog.

An example 25mer UNC13A AON with two spacers and with a phosphodiester bond linking the base at position 3 to a succeeding base can be denoted as:

XXDoXXXXXXXS₁XXXXXXXXXS₂XXXX

where “S₁” represents a first spacer, “S₂” represents a second spacer, “o” represents a phosphodiester bond and “D” represents the base at position 3. Any nucleobase in the AON can be a nucleobase analog.

In some embodiments, nucleoside linkages linking a base at position 4 of a UNC13A AON described herein are phosphodiester bonds. For example, the base at position 4 may be linked to each adjacent base (e.g., preceding base and succeeding base) through a phosphodiester bond. An example 25mer UNC13A AON with phosphodiester bonds linking the base at position 4 can be denoted as:

XXXoDoXXXXXXXXXXXXXXXXXXXXX

where “o” represents a phosphodiester bond and “D” represents the base at position 4. Any nucleobase in the AON can be a nucleobase analog.

In some embodiments, one of the nucleoside linkages linking a base at position 4 of a UNC13A AON described herein is a phosphodiester bond. For example, the base at position 4 may be linked to either the preceding base or the succeeding base through a phosphodiester bond. An example 25mer UNC13A AON with a phosphodiester bond linking the base at position 4 to a preceding base can be denoted as:

XXXoDXXXXXXXXXXXXXXXXXXXXX

where “o” represents a phosphodiester bond and “D” represents the base at position 4. Any nucleobase in the AON can be a nucleobase analog.

An example 25mer UNC13A AON with a phosphodiester bond linking the base at position 4 to a succeeding base can be denoted as:

XXXDoXXXXXXXXXXXXXXXXXXXXX

where “o” represents a phosphodiester bond and “D” represents the base at position 4. Any nucleobase in the AON can be a nucleobase analog.

In some embodiments, nucleoside linkages linking both bases at position 3 and position 4 of a UNC13A AON described herein are phosphodiester bonds. For example, the base at position 3 may be linked to each adjacent base (e.g., preceding base and succeeding base) through a phosphodiester bond, and the base at position 4 may be linked to each adjacent base (e.g., preceding base and succeeding base) through a phosphodiester bond. An example 25mer UNC13A AON with phosphodiester bonds linking the bases at positions 3 and 4 can be denoted as:

XXoDoEoXXXXXXXXXXXXXXXXXXXXX

where “o” represents a phosphodiester bond, “D” represents the base at position 3, and “E” represents the base at position 4. In various embodiments, all other bases of the UNC13A AON are linked through phosphorothioate bonds. Any nucleobase in the AON can be a nucleobase analog.

In various embodiments, UNC13A AON described herein include one or more spacers and phosphodiester bonds are located relative to the one or more spacers. In some embodiments, the Y number of bases immediately preceding a spacer are linked through phosphodiester bonds. In various embodiments, Y is one, two, three, four, five, six, seven, eight, nine, ten, eleven, or twelve bases. In particular embodiments, Y is two bases. For example, if the spacer is located at position 15, the bases at positions 13 and 14 of the UNC13A AON are each linked to their respective adjacent bases through phosphodiester bonds. As described herein, the spacer can be located at various positions in the UNC13A AON and therefore, the 2 bases immediately preceding the spacer can vary within the UNC13A AON depending on where the spacer is situated.

In various embodiments, the UNC13A AON may include more than one spacer. In some embodiments, only one of the spacers has Y number of bases immediately preceding the spacer that are linked through phosphodiester bonds. In such embodiments, the other spacers are linked to respective preceding bases through phosphorothioate bonds. In various embodiments, two of the spacers have Y number of bases immediately preceding the spacers that are linked through phosphodiester bonds. In various embodiments, each of the spacers in the UNC13A AON have Y number of bases immediately preceding the spacers that are linked through phosphodiester bonds. In various embodiments, all other bases of the UNC13A AON are linked through phosphorothioate bonds.

In some embodiments, Y number of bases immediately preceding a spacer and Z number of bases immediately succeeding a spacer are linked through phosphodiester bonds. In various embodiments, Y is one, two, three, four, five, six, seven, eight, nine, ten, eleven, or twelve bases. In various embodiments, Z is one, two, three, four, five, six, seven, eight, nine, ten, eleven, or twelve bases. Y and Z can be independent of each other. In particular embodiments, Y is one base and Z is one base. For example, if the spacer is located at position 15, the bases at positions 14 and 16 of the UNC13A AON are each linked to their respective adjacent bases through phosphodiester bonds. To provide an example, such a UNC13A AON (e.g., 25mer) can be denoted as:

XXXXXXXXXoDoSoEoXXXXXXXXX

where “S” represents a spacer, “o” represents a phosphodiester bond, “D” represents a base immediately preceding the spacer, and “E” represents the base immediately succeeding the spacer. Any nucleobase in the AON can be a nucleobase analog.

As described herein, the spacer can be located at various positions in the UNC13A AON and therefore, the bases immediately preceding or immediately succeeding the spacer can vary within the UNC13A AON depending on where the spacer is situated.

In various embodiments, the UNC13A AON may include more than one spacer. In some embodiments, only one of the spacers has Y number of bases immediately preceding the spacer and Z number of bases immediately succeeding the spacer that are linked through phosphodiester bonds. In such embodiments, the other spacers of the UNC13A AON are linked to respective preceding and succeeding bases through phosphorothioate bonds. To provide an example, such a UNC13A AON (e.g., 25mer) can be denoted as:

XXXXoDoS₁oEoXXXXXXXXS₂XXXXXX

where “S₁” represents a first spacer, “S₂” represents a second spacer, “o” represents a phosphodiester bond, “D” represents a base immediately preceding the spacer, and “E” represents the base immediately succeeding the spacer. Any nucleobase in the AON can be a nucleobase analog.

As another example, such a UNC13A AON (e.g., 25mer) can be denoted as:

XXXXXS₁XXXXXXXoDOS₂oDoXXXXX

where “S₁” represents a first spacer, “S₂” represents a second spacer, “o” represents a phosphodiester bond, “D” represents a base immediately preceding the spacer, and “E” represents the base immediately succeeding the spacer. Any nucleobase in the AON can be a nucleobase analog.

In some embodiments, one of the spacers is linked to the immediately preceding base through a phosphodiester bond. For example, a UNC13A AON includes a first spacer that is linked to the immediately preceding base through a phosphodiester bond, which can be denoted as:

XXXXXXoS₁XXXXXXXXS₂XXXXXX

where “S₁” represents a first spacer, “S₂” represents a second spacer, “o” represents a phosphodiester bond. Any nucleobase in the AON can be a nucleobase analog.

As another example, a UNC13A AON includes a second spacer that is linked to the immediately preceding base through a phosphodiester bond, which can be denoted as:

XXXXXXS₁XXXXXXXXXXXoS₂XXXXXX

where “S₁” represents a first spacer, “S₂” represents a second spacer, “o” represents a phosphodiester bond. Any nucleobase in the AON can be a nucleobase analog.

In various embodiments, the UNC13A AON may be a AON variant (e.g., a 23mer, a 21mer, or a 19mer) where one of the spacers is linked to the immediately preceding base through a phosphodiester bond. For example, the UNC13A AON may be a 21mer with a first spacer that is linked to the immediately preceding base through a phosphodiester bond, which can be denoted as:

XXXXXXXoS₁XXXXXS₂XXXXXXX

where “S₁” represents a first spacer, “S₂” represents a second spacer, “o” represents a phosphodiester bond. Any nucleobase in the AON can be a nucleobase analog.

As another example, the UNC13A AON may be a 21mer with a second spacer that is linked to the immediately preceding base through a phosphodiester bond, which can be denoted as:

XXXXXXXS₁XXXXXoS₂XXXXXXX

where “S₁” represents a first spacer, “S₂” represents a second spacer, “o” represents a phosphodiester bond. Any nucleobase in the AON can be a nucleobase analog.

In some embodiments, the UNC13A AON may be a AON variant (e.g., a 23mer, a 21mer, or a 19mer) where one of the spacers is linked to the immediately preceding base through a phosphodiester bond and the immediately preceding base is further linked to the preceding base through a phosphodiester bond. An example 21mer UNC13A AON can be denoted as:

XXXEoDoS₁XXXXXXS₂XXXXXXX

where “S₁” represents a first spacer, “S₂” represents a second spacer, “o” represents a phosphodiester bond, “D” represents the base immediately preceding S₁ and “E” represents the base immediately preceding “D.” Any nucleobase in the AON can be a nucleobase analog.

As another example, a 21mer UNC13A AON can be denoted as:

XXXXXS₁XXXXEoDoS₂XXXXXXX

where “S₁” represents a first spacer, “S₂” represents a second spacer, “o” represents a phosphodiester bond, “D” represents the base immediately preceding S₂ and “E” represents the base immediately preceding “D.” Any nucleobase in the AON can be a nucleobase analog.

In some embodiments, the UNC13A AON may be a AON variant (e.g., a 23mer, a 21mer, or a 19mer) where a base that immediately precedes a first spacer is linked to another base through a phosphodiester bond. The base that immediately precedes the first spacer may be linked to the first spacer through a non-phosphodiester bond, such as a phosphorothioate bond. Additionally a second spacer is linked to an immediately preceding base through a phosphodiester bond. An example of a 21mer UNC13A AON can be denoted as:

XXXEoDS₁XXXXXXoS₂XXXXXXX

where “S₁” represents a first spacer, “S₂” represents a second spacer, “o” represents a phosphodiester bond, “D” represents the base immediately preceding S₁ and “E” represents the base immediately preceding “D.” Here, the base “D” is linked to the first spacer S₁ through a non-phosphodiester bond (e.g., phosphorothioate bond). Additionally, the base “D” is linked to base “E” through a phosphodiester bond. The second spacer S₂ is linked to an immediately preceding base through a phosphodiester bond. Any nucleobase in the AON can be a nucleobase analog.

Another example of such a 21mer UNC13A AON can be denoted as:

XXXXXoS₁XXXXEoDS₂XXXXXXX

where “S₁” represents a first spacer, “S₂” represents a second spacer, “o” represents a phosphodiester bond, “D” represents the base immediately preceding S₂ and “E” represents the base immediately preceding “D.” Here, the base “D” is linked to the second spacer S₂ through a non-phosphodiester bond (e.g., phosphorothioate bond). Additionally, the base “D” is linked to base “E” through a phosphodiester bond. The first spacer Si is linked to an immediately preceding base through a phosphodiester bond. Any nucleobase in the AON can be a nucleobase analog.

In some embodiments, one of the spacers is linked to the immediately succeeding base through a phosphodiester bond. For example, a UNC13A AON includes a first spacer that is linked to the immediately succeeding base through a phosphodiester bond, which can be denoted as:

XXXXXXS₁oXXXXXXXXXXXS₂XXXXXX

where “S₁” represents a first spacer, “S₂” represents a second spacer, “o” represents a phosphodiester bond. Any nucleobase in the AON can be a nucleobase analog.

As another example, a UNC13A AON includes a second spacer that is linked to the immediately succeeding base through a phosphodiester bond, which can be denoted as:

XXXXXXS₁XXXXXXXXXS₂oXXXXXX

where “S₁” represents a first spacer, “S₂” represents a second spacer, “o” represents a phosphodiester bond. Any nucleobase in the AON can be a nucleobase analog.

In various embodiments, the UNC13A AON may be a AON variant (e.g., a 23mer, a 21mer, or a 19mer) where one of the spacers is linked to the immediately succeeding base through a phosphodiester bond. For example, the UNC13A AON may be a 21mer with a first spacer that is linked to the immediately succeeding base through a phosphodiester bond, which can be denoted as:

XXXXXXXS₁oXXXXXS₂XXXXXXX

where “S₁” represents a first spacer, “S₂” represents a second spacer, “o” represents a phosphodiester bond. Any nucleobase in the AON can be a nucleobase analog.

As another example, the UNC13A AON may be a 21mer with a second spacer that is linked to the immediately succeeding base through a phosphodiester bond, which can be denoted as:

XXXXXXXS₁XXXXXS₂oXXXXXXX

where “S₁” represents a first spacer, “S₂” represents a second spacer, “o” represents a phosphodiester bond. Any nucleobase in the AON can be a nucleobase analog.

In various embodiments, two of the spacers have Y number of bases immediately preceding the spacers and Z number of bases immediately succeeding the spacers that are linked through phosphodiester bonds. In various embodiments, each of the spacers in the UNC13A AON have Y number of bases immediately preceding the spacers and Z number of bases immediately succeeding the spacers that are linked through phosphodiester bonds. An example of such a UNC13A AON (e.g., 25mer) can be denoted as:

XXXXoDoS₁oEoXXXXXXXXXXoFoS₂oHoXXXXX

where “S₁” represents a first spacer, “S₂” represents a second spacer, “o” represents a phosphodiester bond, “D” represents a base immediately preceding the first spacer, “E” represents the base immediately succeeding the first spacer, “F” represents a base immediately preceding the second spacer, and “H” represents the base immediately succeeding the second spacer. In various embodiments, all other bases of the UNC13A AON are linked through phosphorothioate bonds. Any nucleobase in the AON can be a nucleobase analog.

In various UNC13A AON includes two or more spacers and a range of bases located between the two spacers are linked through phosphodiester bonds. In various embodiments, the range of bases include two, three, four, five, six, or seven bases linked through phosphodiester bonds. In particular embodiments, the range of bases include two bases linked through phosphodiester bonds. In particular embodiments, the range of bases include four bases linked through phosphodiester bonds. In various embodiments, all other bases of the UNC13A AON are linked through phosphorothioate bonds. Any nucleobase in the AON can be a nucleobase analog.

In various embodiments, the range of bases linked through phosphodiester bonds are positioned Y number of bases succeeding the first spacer and Z number of preceding the second spacer. In various embodiments, Y is one, two, three, four, five, six, or seven bases. In various embodiments, Z is one, two, three, four, five, six, or seven bases. Y and Z can be independent on each other. Any nucleobase in the AON can be a nucleobase analog.

In particular embodiments, Y is five bases and Z is four bases. To provide an example, such a UNC13A AON (e.g., 25mer) can be denoted as:

XXXXXXXXS₁XXXXoDoEoFoHoXXXS₂XXXX

where “S₁” represents a first spacer, “S₂” represents a second spacer, and “o” represents a phosphodiester bond. The bases “D,” “E,” “F,” and “H” represent the range of bases that are linked through phosphodiester bonds. In this example, the range of bases is located five bases after the first spacer (e.g., D is positioned five bases after the first spacer) and the range of bases is located four bases preceding the second spacer (e.g., H is positioned four bases before the second spacer). Any nucleobase in the AON can be a nucleobase analog.

In particular embodiments, Y is four bases and Z is three bases. To provide an example, such a UNC13A AON (e.g., 23mer can be denoted as:

XXXXXXXS₁XXXoDoEoXXS₂XXXXXXX

where “S₁” represents a first spacer, “S₂” represents a second spacer, and “o” represents a phosphodiester bond. The bases “D” and “E” represent the range of bases that are linked through phosphodiester bonds. In this example, the range of bases is located four bases after the first spacer (e.g., D is positioned four bases after the first spacer) and the range of bases is located three bases preceding the second spacer (e.g., E is positioned three bases before the second spacer). In various embodiments, the positions of the two spacers differ than shown above and therefore, the range of bases linked through phosphodiester bonds are differently positioned. In various embodiments, all other bases of the UNC13A AON are linked through phosphorothioate bonds. Any nucleobase in the AON can be a nucleobase analog.

In some embodiments, a disclosed UNC13A AON may have at least one modified nucleobase, e.g., 5-methylcytosine, and/or at least one methylphosphonate nucleotide, which is placed, for example, either at only one of the 5′ or 3′ ends or at both 5′ and 3′ ends or along the oligonucleotide sequence.

UNC13A AONs may include at least one modified sugar. For example, the sugar moiety of at least one nucleotide constituting the oligonucleotide is a ribose in which the 2′—OH group may be replaced by any one selected from the group consisting of OR, R, R′OR, SH, SR, NH₂, NR₂, N₃, CN, F, Cl, Br, and I (wherein R is an alkyl or aryl and R′ is an alkylene). Examples of a modified sugar moiety include a 2′-OMe modified sugar moiety, bicyclic sugar moiety, 2′-O-(2-methoxyethyl) (2′-MOE or MOE), 2′-O—(N-methylacetamide), 2′-deoxy-2′-fluoro nucleoside, 2′-fluoro-β-D-arabinonucleoside, locked nucleic acid (LNA), constrained ethyl 2′-4′-bridged nucleic acid (cEt), S-cEt, tcDNA, hexitol nucleic acids (HNA), and tricyclic analog (e.g., tcDNA).

In some embodiments, UNC13A AONs comprise 2′OMe (e.g., a UNC13A AON comprising one or more 2′OMe modified sugar), 2′-MOE or MOE (e.g., a UNC13A AON comprising one or more 2′-MOE modified sugar), PNA (e.g., a UNC13A AON comprising one or more N-(2-aminoethyl)-glycine units linked by amide bonds or carbonyl methylene linkage as repeating units in place of a sugar-phosphate backbone), LNA (e.g., a UNC13A AON comprising one or more locked ribose, and can be a mixture of 2′-deoxy nucleotides or 2′OMe nucleotides), c-ET (e.g., a UNC13A AON comprising one or more cET sugar), cMOE (e.g., a UNC13A AON comprising one or more cMOE sugar), morpholino oligomer (e.g., a UNC13A AON comprising a backbone comprising one or more PMO), deoxy-2′-fluoro nucleoside (e.g., a UNC13A AON comprising one or more 2′-fluoro-β-D-arabinonucleoside), tcDNA (e.g., a UNC13A AON comprising one or more tcDNA modified sugar), ENA (e.g., a UNC13A AON comprising one or more ENA modified sugar), or HNA (e.g., a UNC13A AON comprising one or more HNA modified sugar). In some embodiments, a UNC13A AON comprises one or more phosphorothioate linkage, phosphodiester linkage, phosphotriester linkage, methylphosphonate linkage, phosphoramidate linkage, a phosphoramidothioate linkage, a thiophosphorodiamidate linkage, morpholino linkage, PNA linkage, or any combination of phosphorothioate linkage, phosphodiester linkage, a phosphotriester linkage, methylphosphonate linkage, phosphoramidate linkage, morpholino linkage, and PNA linkage. In some embodiments, a UNC13A AON comprises one or more phosphorothioate linkage, phosphodiester linkage, or a combination of phosphorothioate and phosphodiester linkages.

In some embodiments, UNC13A AONs with a sequence of any one of SEQ ID NOs: 4531-5794, SEQ ID NO: 7059-8322, SEQ ID NOs: 9596-9696, or SEQ ID NOs: 10670-10779 is a chirally controlled oligonucleotide, such as a chirally controlled oligonucleotide described in any of U.S. Pat. Nos. 9,982,257, 10,590,413, 10,724,035, 10,450,568, and PCT Publication No. WO2019200185, each of which is hereby incorporated by reference in its entirety.

For example, a UNC13A AON with a sequence of any one of SEQ ID NOs: 4531-5794, SEQ ID NO: 7059-8322, SEQ ID NOs: 9596-9696, or SEQ ID NOs: 10670-10779 is a chirally controlled oligonucleotide comprising a plurality of oligonucleotides of at least one type, wherein each type is defined by: 1) base sequence: 2) pattern of backbone linkages, 3) pattern of backbone chiral centers; and 4) pattern of backbone X-moieties (—X-L-R¹); wherein: the oligonucleotides of the at least one type comprise one or more phosphorothioate triester internucleotidic linkages and one or more phosphate diester linkage; the oligonucleotides of the at least one type comprise at least two consecutive modified internucleotidic linkages; and oligonucleotides of the at least one oligonucleotide type comprise one or more modified internucleotidic linkages independently having the structure of:

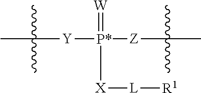

wherein: P* is an asymmetric phosphorus atom and is either Rp or Sp;
W is O, S or Se; each of X, Y and Z is independently —O—, —S—, —N(-L-R¹)—, or L; L is a covalent bond or an optionally substituted, linear or branched C₁-C₅₀ alkylene, wherein one or more methylene units of L are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—; R¹ is halogen, R, or an optionally substituted C₁-C₁₀ aliphatic wherein one or more methylene units are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—; each R′ is independently —R, —C(O)R, —CO₂R, or —SO₂R, or: two R′ on the same nitrogen are taken together with their intervening atoms to form an optionally substituted heterocyclic or heteroaryl ring, or two R′ on the same carbon are taken together with their intervening atoms to form an optionally substituted aryl, carbocyclic, heterocyclic, or heteroaryl ring; -Cy- is an optionally substituted bivalent ring selected from phenylene, carbocyclylene, arylene, heteroarylene, or heterocyclylene; each R is independently hydrogen, or an optionally substituted group selected from C₁-C₆ aliphatic, phenyl, carbocyclyl, aryl, heteroaryl, or heterocyclyl, and each

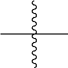

independently represents a connection to a nucleoside. In some embodiments, a UNC13A AON with a sequence of any one of SEQ ID NOs: 4531-5794, SEQ ID NO: 7059-8322, SEQ ID NOs: 9596-9696, or SEQ ID NOs: 10670-10779 is a chirally controlled oligonucleotide comprising certain chemical modifications (e.g., 2′F (2′ Fluoro, which contains a fluorine molecule at the 2′ ribose position (instead of 2′-hydroxyl group in an RNA monomer)), 2′-OMe, phosphorothioate linkages, lipid conjugation, etc.), as described in U.S. Pat. No. 10,450,568.

### SMN2

In some embodiments, all of the nucleotide linkages of a SMN2 AON of any of SEQ ID NOs: 9710-10141, SEQ ID NOs: 10574-10651, and SEQ ID NOs: 10783-10808 are phosphorothioate linkages. In some embodiments, one or more of the nucleotide linkages of a SMN2 AON of any of SEQ ID NOs: 9710-10141, SEQ ID NOs: 10574-10651, and SEQ ID NOs: 10783-10808 are phosphorothioate linkages.

In various embodiments, nucleotide linkages of SMN2 AON described herein such as any of SEQ ID NOs: 9710-10141, SEQ ID NOs: 10574-10651, and SEQ ID NOs: 10783-10808 include a mix of phosphodiester and phosphorothioate linkages.

In some embodiments, nucleoside linkages linking a base at position 3 of a SMN2 AON described herein are phosphodiester bonds. For example, the base at position 3 may be linked to each adjacent base (e.g., preceding base and succeeding base) through a phosphodiester bond. An example 25mer SMN2 AON with phosphodiester bonds linking the base at position 3 can be denoted as:

XXoDoXXXXXXXXXXXXXXXXXXXXXX

where “o” represents a phosphodiester bond and “D” represents the base at position 3. Any nucleobase in the AON can be a nucleobase analog.

In some embodiments, one of the nucleoside linkages linking a base at position 3 of a SMN2 AON described herein is a phosphodiester bond. For example, the base at position 3 may be linked to either the preceding base or the succeeding base through a phosphodiester bond. An example 25mer SMN2 AON with a phosphodiester bond linking the base at position 3 to a preceding base can be denoted as:

XXoDXXXXXXXXXXXXXXXXXXXXXX

where “o” represents a phosphodiester bond and “D” represents the base at position 3. Any nucleobase in the AON can be a nucleobase analog.

An example 25mer SMN2 AON with a phosphodiester bond linking the base at position 3 to a succeeding base can be denoted as:

XXDoXXXXXXXXXXXXXXXXXXXXXX

where “o” represents a phosphodiester bond and “D” represents the base at position 3. Any nucleobase in the AON can be a nucleobase analog.

In various embodiments, in addition to one of the nucleoside linkages linking a base at position 3 of a SMN2 AON described herein being a phosphodiester bond, the SMN2 AON further includes two spacers. The two spacers can be positioned in the SMN2 AON such that the SMN2 AON includes a segment with at most 7 linked nucleosides. An example 25mer SMN2 AON with two spacers and with a phosphodiester bond linking the base at position 3 to a preceding base can be denoted as:

XxoDS₁XXXXXXXXXS₂XXXXXXXXXXX

where “S₁” represents a first spacer, “S₂” represents a second spacer, “o” represents a phosphodiester bond and “D” represents the base at position 3. Any nucleobase in the AON can be a nucleobase analog.

An example 25mer SMN2 AON with two spacers and with a phosphodiester bond linking the base at position 3 to a succeeding base can be denoted as:

XXDoXXXXXXXS₁XXXXXXXXXS₂XXXX

where “S₁” represents a first spacer, “S₂” represents a second spacer, “o” represents a phosphodiester bond and “D” represents the base at position 3. Any nucleobase in the AON can be a nucleobase analog.

In some embodiments, nucleoside linkages linking a base at position 4 of a SMN2 AON described herein are phosphodiester bonds. For example, the base at position 4 may be linked to each adjacent base (e.g., preceding base and succeeding base) through a phosphodiester bond. An example 25mer SMN2 AON with phosphodiester bonds linking the base at position 4 can be denoted as:

XXXoDoXXXXXXXXXXXXXXXXXXXXX

where “o” represents a phosphodiester bond and “D” represents the base at position 4. Any nucleobase in the AON can be a nucleobase analog.

In some embodiments, one of the nucleoside linkages linking a base at position 4 of a SMN2 AON described herein is a phosphodiester bond. For example, the base at position 4 may be linked to either the preceding base or the succeeding base through a phosphodiester bond. An example 25mer SMN2 AON with a phosphodiester bond linking the base at position 4 to a preceding base can be denoted as:

XXXoDXXXXXXXXXXXXXXXXXXXXX

where “o” represents a phosphodiester bond and “D” represents the base at position 4. Any nucleobase in the AON can be a nucleobase analog.

An example 25mer SMN2 AON with a phosphodiester bond linking the base at position 4 to a succeeding base can be denoted as:

XXXDoXXXXXXXXXXXXXXXXXXXXX

where “o” represents a phosphodiester bond and “D” represents the base at position 4. Any nucleobase in the AON can be a nucleobase analog.

In some embodiments, nucleoside linkages linking both bases at position 3 and position 4 of a SMN2 AON described herein are phosphodiester bonds. For example, the base at position 3 may be linked to each adjacent base (e.g., preceding base and succeeding base) through a phosphodiester bond, and the base at position 4 may be linked to each adjacent base (e.g., preceding base and succeeding base) through a phosphodiester bond. An example 25mer SMN2 AON with phosphodiester bonds linking the bases at positions 3 and 4 can be denoted as:

XXoDoEoXXXXXXXXXXXXXXXXXXXXX

where “o” represents a phosphodiester bond, “D” represents the base at position 3, and “E” represents the base at position 4. In various embodiments, all other bases of the SMN2 AON are linked through phosphorothioate bonds. Any nucleobase in the AON can be a nucleobase analog.

In various embodiments, SMN2 AON described herein include one or more spacers and phosphodiester bonds are located relative to the one or more spacers. In some embodiments, the Y number of bases immediately preceding a spacer are linked through phosphodiester bonds. In various embodiments, Y is one, two, three, four, five, six, seven, eight, nine, ten, eleven, or twelve bases. In particular embodiments, Y is two bases. For example, if the spacer is located at position 15, the bases at positions 13 and 14 of the SMN2 AON are each linked to their respective adjacent bases through phosphodiester bonds. As described herein, the spacer can be located at various positions in the SMN2 AON and therefore, the 2 bases immediately preceding the spacer can vary within the SMN2 AON depending on where the spacer is situated.

In various embodiments, the SMN2 AON may include more than one spacer. In some embodiments, only one of the spacers has Y number of bases immediately preceding the spacer that are linked through phosphodiester bonds. In such embodiments, the other spacers are linked to respective preceding bases through phosphorothioate bonds. In various embodiments, two of the spacers have Y number of bases immediately preceding the spacers that are linked through phosphodiester bonds. In various embodiments, each of the spacers in the SMN2 AON have Y number of bases immediately preceding the spacers that are linked through phosphodiester bonds. In various embodiments, all other bases of the SMN2 AON are linked through phosphorothioate bonds.

In some embodiments, Y number of bases immediately preceding a spacer and Z number of bases immediately succeeding a spacer are linked through phosphodiester bonds. In various embodiments, Y is one, two, three, four, five, six, seven, eight, nine, ten, eleven, or twelve bases. In various embodiments, Z is one, two, three, four, five, six, seven, eight, nine, ten, eleven, or twelve bases. Y and Z can be independent of each other. In particular embodiments, Y is one base and Z is one base. For example, if the spacer is located at position 15, the bases at positions 14 and 16 of the SMN2 AON are each linked to their respective adjacent bases through phosphodiester bonds. To provide an example, such a SMN2 AON (e.g., 25mer) can be denoted as:

XXXXXXXXXXXXXoDoSoEoXXXXXXXXX

where “S” represents a spacer, “o” represents a phosphodiester bond, “D” represents a base immediately preceding the spacer, and “E” represents the base immediately succeeding the spacer. Any nucleobase in the AON can be a nucleobase analog.

As described herein, the spacer can be located at various positions in the SMN2 AON and therefore, the bases immediately preceding or immediately succeeding the spacer can vary within the SMN2 AON depending on where the spacer is situated.

In various embodiments, the SMN2 AON may include more than one spacer. In some embodiments, only one of the spacers has Y number of bases immediately preceding the spacer and Z number of bases immediately succeeding the spacer that are linked through phosphodiester bonds. In such embodiments, the other spacers of the SMN2 AON are linked to respective preceding and succeeding bases through phosphorothioate bonds. To provide an example, such a SMN2 AON (e.g., 25mer) can be denoted as:

XXXXoDoS₁oEoXXXXXXXXXXXS₂XXXXXX

where “S₁” represents a first spacer, “S₂” represents a second spacer, “o” represents a phosphodiester bond, “D” represents a base immediately preceding the spacer, and “E” represents the base immediately succeeding the spacer. Any nucleobase in the AON can be a nucleobase analog.

As another example, such a SMN2 AON (e.g., 25mer) can be denoted as:

XXXXXS₁XXXXXXXXXXXoDoS₂oDoXXXXX

where “S₁” represents a first spacer, “S₂” represents a second spacer, “o” represents a phosphodiester bond, “D” represents a base immediately preceding the spacer, and “E” represents the base immediately succeeding the spacer. Any nucleobase in the AON can be a nucleobase analog.

In some embodiments, one of the spacers is linked to the immediately preceding base through a phosphodiester bond. For example, a SMN2 AON includes a first spacer that is linked to the immediately preceding base through a phosphodiester bond, which can be denoted as:

XXXXXXoS₁XXXXXXXXXXXS₂XXXXXX

where “S₁” represents a first spacer, “S₂” represents a second spacer, “o” represents a phosphodiester bond. Any nucleobase in the AON can be a nucleobase analog.

As another example, a SMN2 AON includes a second spacer that is linked to the immediately preceding base through a phosphodiester bond, which can be denoted as:

XXXXXXS₁XXXXXXXXXXXoS₂XXXXXX

where “S₁” represents a first spacer, “S₂” represents a second spacer, “o” represents a phosphodiester bond. Any nucleobase in the AON can be a nucleobase analog.

In various embodiments, the SMN2 AON may be a AON variant (e.g., a 23mer, a 21mer, or a 19mer) where one of the spacers is linked to the immediately preceding base through a phosphodiester bond. For example, the SMN2 AON may be a 21mer with a first spacer that is linked to the immediately preceding base through a phosphodiester bond, which can be denoted as:

XXXXXXXoS₁XXXXXS₂XXXXXXX

where “S₁” represents a first spacer, “S₂” represents a second spacer, “o” represents a phosphodiester bond. Any nucleobase in the AON can be a nucleobase analog.

As another example, the SMN2 AON may be a 21mer with a second spacer that is linked to the immediately preceding base through a phosphodiester bond, which can be denoted as:

XXXXXXXS₁XXXXXoS₂XXXXXXX

where “S₁” represents a first spacer, “S₂” represents a second spacer, “o” represents a phosphodiester bond. Any nucleobase in the AON can be a nucleobase analog.

In some embodiments, the SMN2 AON may be a AON variant (e.g., a 23mer, a 21mer, or a 19mer) where one of the spacers is linked to the immediately preceding base through a phosphodiester bond and the immediately preceding base is further linked to the preceding base through a phosphodiester bond. An example 21mer SMN2 AON can be denoted as:

XXXEoDoS₁XXXXXXS₂XXXXXXX

where “S₁” represents a first spacer, “S₂” represents a second spacer, “o” represents a phosphodiester bond, “D” represents the base immediately preceding S₁ and “E” represents the base immediately preceding “D.” Any nucleobase in the AON can be a nucleobase analog.

As another example, a 21mer SMN2 AON can be denoted as:

XXXXXS₁XXXXEoDoS₂XXXXXXX

where “S₁” represents a first spacer, “S₂” represents a second spacer, o represents a phosphodiester bond, “D” represents the base immediately preceding S₂ and “E” represents the base immediately preceding “D.” Any nucleobase in the AON can be a nucleobase analog.

In some embodiments, the SMN2 AON may be a AON variant (e.g., a 23mer, a 21mer, or a 19mer) where a base that immediately precedes a first spacer is linked to another base through a phosphodiester bond. The base that immediately precedes the first spacer may be linked to the first spacer through a non-phosphodiester bond, such as a phosphorothioate bond. Additionally a second spacer is linked to an immediately preceding base through a phosphodiester bond. An example of a 21mer SMN2 AON can be denoted as:

XXXEoDS₁XXXXXXoS₂XXXXXXX

where “S₁” represents a first spacer, “S₂” represents a second spacer, “o” represents a phosphodiester bond, “D” represents the base immediately preceding S₁ and “E” represents the base immediately preceding “D.” Here, the base “D” is linked to the first spacer S₁ through a non-phosphodiester bond (e.g., phosphorothioate bond). Additionally, the base “D” is linked to base “E” through a phosphodiester bond. The second spacer S₂ is linked to an immediately preceding base through a phosphodiester bond. Any nucleobase in the AON can be a nucleobase analog.

Another example of such a 21mer SMN2 AON can be denoted as:

XXXXXoS₁XXXXEoDS₂XXXXXXX

where “S₁” represents a first spacer, “S₂” represents a second spacer, “o” represents a phosphodiester bond, “D” represents the base immediately preceding S₂ and “E” represents the base immediately preceding “D.” Here, the base “D” is linked to the second spacer S₂ through a non-phosphodiester bond (e.g., phosphorothioate bond). Additionally, the base “D” is linked to base “E” through a phosphodiester bond. The first spacer Si is linked to an immediately preceding base through a phosphodiester bond. Any nucleobase in the AON can be a nucleobase analog.

In some embodiments, one of the spacers is linked to the immediately succeeding base through a phosphodiester bond. For example, a SMN2 AON includes a first spacer that is linked to the immediately succeeding base through a phosphodiester bond, which can be denoted as:

XXXXXXS₁oXXXXXXXXXXS₂XXXXXX

where “S₁” represents a first spacer, “S₂” represents a second spacer, “o” represents a phosphodiester bond. Any nucleobase in the AON can be a nucleobase analog.

As another example, a SMN2 AON includes a second spacer that is linked to the immediately succeeding base through a phosphodiester bond, which can be denoted as:

XXXXXXS₁XXXXXXXXXXXS₂oXXXXXX

where “S₁” represents a first spacer, “S₂” represents a second spacer, “o” represents a phosphodiester bond. Any nucleobase in the AON can be a nucleobase analog.

In various embodiments, the SMN2 AON may be a AON variant (e.g., a 23mer, a 21mer, or a 19mer) where one of the spacers is linked to the immediately succeeding base through a phosphodiester bond. For example, the SMN2 AON may be a 21mer with a first spacer that is linked to the immediately succeeding base through a phosphodiester bond, which can be denoted as:

XXXXXXXS₁oXXXXXS₂XXXXXXX

where “S₁” represents a first spacer, “S₂” represents a second spacer, “o” represents a phosphodiester bond. Any nucleobase in the AON can be a nucleobase analog.

As another example, the SMN2 AON may be a 21mer with a second spacer that is linked to the immediately succeeding base through a phosphodiester bond, which can be denoted as:

XXXXXXXS₁XXXXXS₂oXXXXXXX

where “S₁” represents a first spacer, “S₂” represents a second spacer, “o” represents a phosphodiester bond. Any nucleobase in the AON can be a nucleobase analog.

In various embodiments, two of the spacers have Y number of bases immediately preceding the spacers and Z number of bases immediately succeeding the spacers that are linked through phosphodiester bonds. In various embodiments, each of the spacers in the SMN2 AON have Y number of bases immediately preceding the spacers and Z number of bases immediately succeeding the spacers that are linked through phosphodiester bonds. An example of such a SMN2 AON (e.g., 25mer) can be denoted as:

XXXXoDoS₁oEoXXXXXXXXXXoFoS₂oHoXXXXX

where “S₁” represents a first spacer, “S₂” represents a second spacer, “o” represents a phosphodiester bond, “D” represents a base immediately preceding the first spacer, “E” represents the base immediately succeeding the first spacer, “F” represents a base immediately preceding the second spacer, and “H” represents the base immediately succeeding the second spacer. In various embodiments, all other bases of the SMN2 AON are linked through phosphorothioate bonds. Any nucleobase in the AON can be a nucleobase analog.

In various SMN2 AON includes two or more spacers and a range of bases located between the two spacers are linked through phosphodiester bonds. In various embodiments, the range of bases include two, three, four, five, six, or seven bases linked through phosphodiester bonds. In particular embodiments, the range of bases include two bases linked through phosphodiester bonds. In particular embodiments, the range of bases include four bases linked through phosphodiester bonds. In various embodiments, all other bases of the SMN2 AON are linked through phosphorothioate bonds. Any nucleobase in the AON can be a nucleobase analog.

In various embodiments, the range of bases linked through phosphodiester bonds are positioned Y number of bases succeeding the first spacer and Z number of preceding the second spacer. In various embodiments, Y is one, two, three, four, five, six, or seven bases. In various embodiments, Z is one, two, three, four, five, six, or seven bases. Y and Z can be independent on each other. Any nucleobase in the AON can be a nucleobase analog.

In particular embodiments, Y is five bases and Z is four bases. To provide an example, such a SMN2 AON (e.g., 25mer) can be denoted as:

XXXXXXXXS₁XXXXoDoEoFoHoXXXS₂XXXX

where “S₁” represents a first spacer, “S₂” represents a second spacer, and “o” represents a phosphodiester bond. The bases “D,” “E,” “F,” and “H” represent the range of bases that are linked through phosphodiester bonds. In this example, the range of bases is located five bases after the first spacer (e.g., D is positioned five bases after the first spacer) and the range of bases is located four bases preceding the second spacer (e.g., H is positioned four bases before the second spacer). Any nucleobase in the AON can be a nucleobase analog.

In particular embodiments, Y is four bases and Z is three bases. To provide an example, such a SMN2 AON (e.g., 23mer) can be denoted as:

XXXXXXXS₁XXXoDoEoXXS₂XXXXXXX

where “S₁” represents a first spacer, “S₂” represents a second spacer, and “o” represents a phosphodiester bond. The bases “D” and “E” represent the range of bases that are linked through phosphodiester bonds. In this example, the range of bases is located four bases after the first spacer (e.g., D is positioned four bases after the first spacer) and the range of bases is located three bases preceding the second spacer (e.g., E is positioned three bases before the second spacer). In various embodiments, the positions of the two spacers differ than shown above and therefore, the range of bases linked through phosphodiester bonds are differently positioned. In various embodiments, all other bases of the SMN2 AON are linked through phosphorothioate bonds. Any nucleobase in the AON can be a nucleobase analog.

In some embodiments, a disclosed SMN2 AON may have at least one modified nucleobase, e.g., 5-methylcytosine, and/or at least one methylphosphonate nucleotide, which is placed, for example, either at only one of the 5′ or 3′ ends or at both 5′ and 3′ ends or along the oligonucleotide sequence.

SMN2 AONs may include at least one modified sugar. For example, the sugar moiety of at least one nucleotide constituting the oligonucleotide is a ribose in which the 2′—OH group may be replaced by any one selected from the group consisting of OR, R, R′OR, SH, SR, NH₂, NR₂, N₃, CN, F, Cl, Br, and I (wherein R is an alkyl or aryl and R′ is an alkylene). Examples of a modified sugar moiety include a 2′-OMe modified sugar moiety, bicyclic sugar moiety, 2′-O-(2-methoxyethyl) (2′-MOE or MOE), 2′-O—(N-methylacetamide), 2′-deoxy-2′-fluoro nucleoside, 2′-fluoro-β-D-arabinonucleoside, locked nucleic acid (LNA), constrained ethyl 2′-4′-bridged nucleic acid (cEt), S-cEt, tcDNA, hexitol nucleic acids (HNA), and tricyclic analog (e.g., tcDNA).

In some embodiments, SMN2 AONs comprise 2′OMe (e.g., a SMN2 AON comprising one or more 2′OMe modified sugar), 2′-MOE or MOE (e.g., a SMN2 AON comprising one or more 2′-MOE modified sugar), PNA (e.g., a SMN2 AON comprising one or more N-(2-aminoethyl)-glycine units linked by amide bonds or carbonyl methylene linkage as repeating units in place of a sugar-phosphate backbone), LNA (e.g., a SMN2 AON comprising one or more locked ribose, and can be a mixture of 2′-deoxy nucleotides or 2′OMe nucleotides), c-ET (e.g., a SMN2 AON comprising one or more cET sugar), cMOE (e.g., a SMN2 AON comprising one or more cMOE sugar), morpholino oligomer (e.g., a SMN2 AON comprising a backbone comprising one or more PMO), deoxy-2′-fluoro nucleoside (e.g., a SMN2 AON comprising one or more 2′-fluoro-β-D-arabinonucleoside), tcDNA (e.g., a SMN2 AON comprising one or more tcDNA modified sugar), ENA (e.g., a SMN2 AON comprising one or more ENA modified sugar), or HNA (e.g., a SMN2 AON comprising one or more HNA modified sugar). In some embodiments, a SMN2 AON comprises one or more phosphorothioate linkage, phosphodiester linkage, phosphotriester linkage, methylphosphonate linkage, phosphoramidate linkage, a phosphoramidothioate linkage, a thiophosphorodiamidate linkage, morpholino linkage, PNA linkage, or any combination of phosphorothioate linkage, phosphodiester linkage, a phosphotriester linkage, methylphosphonate linkage, phosphoramidate linkage, morpholino linkage, and PNA linkage. In some embodiments, a SMN2 AON comprises one or more phosphorothioate linkage, phosphodiester linkage, or a combination of phosphorothioate and phosphodiester linkages.

In some embodiments, SMN2 AONs with a sequence of any one of SEQ ID NOs: 9710-10141, SEQ ID NOs: 10574-10651, and SEQ ID NOs: 10783-10808 is a chirally controlled oligonucleotide, such as a chirally controlled oligonucleotide described in any of U.S. Pat. Nos. 9,982,257, 10,590,413, 10,724,035, 10,450,568, and PCT Publication No. WO2019200185, each of which is hereby incorporated by reference in its entirety.

For example, a SMN2 AON with a sequence of any one of SEQ ID NOs: 9710-10141, SEQ ID NOs: 10574-10651, and SEQ ID NOs: 10783-10808 is a chirally controlled oligonucleotide comprising a plurality of oligonucleotides of at least one type, wherein each type is defined by: 1) base sequence; 2) pattern of backbone linkages; 3) pattern of backbone chiral centers, and 4) pattern of backbone X-moieties (—X-L-R¹); wherein: the oligonucleotides of at least one type comprise one or more phosphorothioate triester internucleotidic linkages and one or more phosphate diester linkage; the oligonucleotides of at least one type comprise at least two consecutive modified internucleotidic linkages; and oligonucleotides of at least one oligonucleotide type comprise one or more modified internucleotidic linkages independently having the structure of:

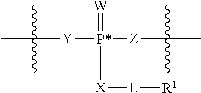

wherein: P* is an asymmetric phosphorus atom and is either Rp or Sp;
W is O, S or Se; each of X, Y and Z is independently —O—, —S—, —N(-L-R¹)—, or L; L is a covalent bond or an optionally substituted, linear or branched C₁-C₅₀ alkylene, wherein one or more methylene units of L are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—; R¹ is halogen, R, or an optionally substituted C₁-C₁₀ aliphatic wherein one or more methylene units are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—; each R′ is independently —R, —C(O)R, —CO₂R, or —SO₂R, or: two R′ on the same nitrogen are taken together with their intervening atoms to form an optionally substituted heterocyclic or heteroaryl ring, or two R′ on the same carbon are taken together with their intervening atoms to form an optionally substituted aryl, carbocyclic, heterocyclic, or heteroaryl ring; -Cy- is an optionally substituted bivalent ring selected from phenylene, carbocyclylene, arylene, heteroarylene, or heterocyclylene; each R is independently hydrogen, or an optionally substituted group selected from C₁-C₆ aliphatic, phenyl, carbocyclyl, aryl, heteroaryl, or heterocyclyl; and each

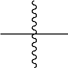

independently represents a connection to a nucleoside. In some embodiments, a SMN2 AON with a sequence of any one of SEQ ID NOs: 9710-10141, SEQ ID NOs: 10574-10651, and SEQ ID NOs: 10783-10808 is a chirally controlled oligonucleotide comprising certain chemical modifications (e.g., 2′F (2′ Fluoro, which contains a fluorine molecule at the 2′ ribose position (instead of 2′-hydroxyl group in an RNA monomer)), 2′-OMe, phosphorothioate linkages, lipid conjugation, etc.), as described in U.S. Pat. No. 10,450,568.

### Motor Neuron Diseases

Motor neuron diseases are a group of diseases characterized by loss of function of motor neurons that coordinate voluntary movement of muscles by the brain. Motor neuron diseases may affect upper and/or lower motor neurons, and may have sporadic or familial origins. Motor neuron diseases include amyotrophic lateral sclerosis (ALS or Lou Gehrig's disease), progressive bulbar palsy, pseudobulbar palsy, progressive muscular atrophy, primary lateral sclerosis, spinal muscular atrophy, post-polio syndrome, and ALS with frontotemporal dementia.

Symptoms of motor neuron diseases include muscle decay or weakening, muscle pain, spasms, slurred speech, difficulty swallowing, loss of muscle control, joint pain, stiff limbs, difficulty breathing, drooling, and complete loss of muscle control, including over basic functions such as breathing, swallowing, eating, speaking, and limb movement. These symptoms are also sometimes accompanied by depression, loss of memory, difficulty with planning, language deficits, altered behavior, and difficulty assessing spatial relationships and/or changes in personality.

Motor neuron diseases can be assessed and diagnosed by a clinician of skill, for example, a neurologist, using various tools and tests. For example, the presence or risk of developing a motor neuron disease can be assessed or diagnosed using blood and urine tests (for example, tests that assay for the presence of creatinine kinase), magnetic resonance imaging (MRI), electromyography (EMG), nerve conduction study (NCS), spinal tap, lumbar puncture, and/or muscle biopsy. Motor neuron diseases can be diagnosed with the aid of a physical exam and/or a neurological exam to assess motor and sensory skills, nerve function, hearing and speech, vision, coordination and balance, mental status, and changes in mood or behavior.

### Amyotrophic Lateral Sclerosis

ALS is a progressive motor neuron disease that disrupts signals to all voluntary muscles. ALS results in atrophy of both upper and lower motor neurons. Symptoms of ALS include weakening and wasting of the bulbar muscles, general and bilateral loss of strength, spasticity, muscle spasms, muscle cramps, fasciculations, slurred speech, and difficulty breathing or loss of ability to breathe. Some individuals with ALS also suffer from cognitive decline. At the molecular level, ALS is characterized by protein and RNA aggregates in the cytoplasm of motor neurons, including aggregates of the RNA-binding protein TDP43.

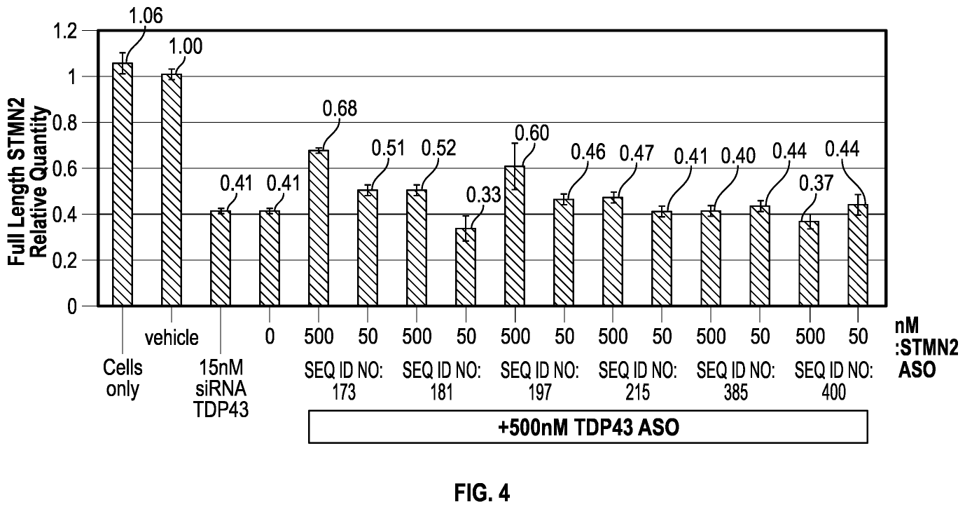

ALS is most common in males above 40 years of age, although it can also occur in women and children. Risk of ALS is also heightened in individuals who smoke, are exposed to chemicals such as lead, or who have served in the military. Most instances of ALS are sporadic, while only about 10% of cases are familial. Causes of ALS include sporadic or inherited genetic mutations, high levels of glutamate, protein mishandling. Genetic mutations associated with ALS include mutations in the genes SOD1, C9orf72, TARDBP, FUS, ANG, ATXN2, CHCHD10, CHMP2B, DCTN1, ErbB4, FIG. 4, HNRPA1, MATR3, NEFH, OPTN, PFN1, PRPH, SETX, SIGMAR1, SMN1, SPG11, SQSTM1, TBK1, TRPM7, TUBA4A, UBQLN2, VAPB, and VCP.

### Frontotemporal Dementia

Frontotemporal dementia (FTD) is a form of dementia that affects the frontal and temporal lobes of the brain. FTD includes frontotemporal lobar degeneration (FTLD). It has an earlier average age of onset than Alzheimer's disease—40 years of age. Symptoms of FTD include extreme changes in behavior and personality, speech and language problems, and movement-related symptoms such as tremor, rigidity, muscle spasm, weakness, and difficulty swallowing. Subtypes of FTD include behavior variant frontotemporal dementia (bvFTD), characterized by changes in personality and behavior, and primary progressive aphasia (PPA), which affects language skills, speaking, writing and comprehension. FTD is associated with tau protein accumulation (Pick bodies) and altered TDP43 function. About 30% of cases of FTD are familial, and no other risk factors other than family history of the disease are known. Genetic mutations associated with FTD include mutations in the genes C9orf72, Progranulin (GRN), microtubule-associated protein tau (MAPT), UBQLN2, VPC, CHMP2B, TARDBP, FUS, ITM2B, CHCHD10, SQSTM1, PSEN1, PSEN2, CTSF, CYP27A1, TBK1 and TBP.

Amyotrophic Lateral Sclerosis with Frontotemporal Dementia

Amyotrophic lateral sclerosis with frontotemporal dementia (ALS with FTD) is a clinical syndrome in which FTD and ALS occur in the same individual. Interestingly, mutations in C9orf72 are the most common cause of familial forms of ALS and FTD. Additionally, mutations in TBK1, VCP, SQSTM1, UBQLN2 and CHMP2B are also associated with ALS with FTD. Symptoms of ALS with FTD include dramatic changes in personality, as well as muscle weakness, muscle atrophy, fasciculations, spasticity, dysarthria, dysphagia, and degeneration of the spinal cord, motor neurons, and frontal and temporal lobes of the brain. At the molecular level, ALS with FTD is characterized by the accumulation of TDP-43 and/or FUS proteins. TBK1 mutations are associated with ALS, FTD, and ALS with FTD.

### Limbic-Predominant Age-Related TDP-43 Encephalopathy (LATE)

Limbic-predominant age-related TDP-43 encephalopathy (LATE) is characterized by accumulation of misfolded TDP-43 protein in the brain, specifically in the limbic system. LATE is a neurological disorder that typically manifests in older patients (e.g., greater than 80 years old). LATE can be a diagnosis for dementia and LATE often mimics the symptoms of Alzheimer's Disease including memory loss, confusion, and mood changes.

### Spinal Muscular Atrophy

Spinal muscular atrophy (SMA) is a group of genetic neuromuscular disorders that affect the nerve cells that control voluntary muscles (motor neurons). The loss of motor neurons causes progressive muscle weakness and loss of movement due to muscle wasting (atrophy). The severity of the symptoms, the age at which symptoms begin, and genetic cause varies by type. Many types of SMA mainly affect the muscles involved in walking, sitting, arm movement, and head control. Breathing and swallowing may also become difficult as the disease progresses in many types of SMA. In some types of SMA, the loss of motor neurons makes it hard to control movement of the hands and feet.

In individuals affected by SMA, the SMN1 gene is mutated in such a way that it is unable to correctly code the SMN protein—due to either a deletion occurring at exon 7 or to other point mutations. There is no complete cure for SMA. Treatment consists of managing the symptoms and preventing complications. There are three approved therapies for treating SMA: of nusinersen (spinraza), onasemnogene abeparvovec-xioi (zolgensma), and risdiplam (evrysdi).

### Methods of Treatment—STMN2

The disclosure contemplates, in part, treating neurological diseases including any of amyotrophic lateral sclerosis (ALS), frontotemporal dementia (FTD), Alzheimer's disease (AD), Parkinson's disease (PD), Huntington's disease, progressive supranuclear palsy (PSP), brain trauma, spinal cord injury, corticobasal degeneration (CBD), and Limbic-predominant age-related TDP-43 encephalopathy (LATE) in a patient in need thereof comprising administering a STMN2 AON. In some embodiments, provided herein are methods for treatment of a neurological disease in a patient in need thereof, comprising administering a disclosed STMN2 AON. In some embodiments of the disclosure, an effective amount of a disclosed STMN2 oligonucleotide may be administered to a patient in need thereof to treat a neurological disease, and/or to increase, restore, or stabilize expression of STMN2 mRNA that is capable of translation to produce a functional STMN2 protein, thereby increase, restore, or stabilize STMN2 activity and/or function.

In some embodiments, treating a neurological disease comprises at least ameliorating or reducing one symptom associated with the neurological disease (for example, reducing muscle weakness in a patient with ALS). Methods of treating a neurological disease (for example, ALS, FTD, or ALS with FTD) in a patient suffering therefrom are provided, that include administering a disclosed STMN2 AON. In some embodiments, methods of slowing the progression of a neurological disease, for example, a motor neuron disease, are provided.

Provided herein are methods of treating, reducing the risk of developing, or delaying the onset of a neurological disease in a subject in need thereof comprising administering a disclosed STMN2 AON. The methods include for example, treating a subject at risk of developing a neurological disease; e.g., administering to the subject an effective amount of a disclosed STMN2 AON. Neurological diseases that can be treated in this manner include motor neuron diseases, ALS, FTD, ALS with FTD, progressive bulbar palsy, pseudobulbar palsy, progressive muscular atrophy, primary lateral sclerosis, spinal muscular atrophy, and post-polio syndrome.

Methods of preventing or treating neurological diseases (for example, PD, ALS, FTD, and ALS with FTD) form part of this disclosure. Such methods may comprise administering to a patient in need thereof or a patient at risk, a pharmaceutical preparation comprising a STMN2 AON disclosed herein. For example, a method of preventing or treating a neurological disease is provided comprising administering to a patient in need thereof a STMN2 AON disclosed herein.

Patients treated using an above method may experience an increase, restoration of, or stabilization of STMN2 mRNA expression, which is capable of translation to produce a functional STMN2 protein, of at least about 5%, 10%, 20%, 30%, 40% or even 50%, thereby increase, restore, or stabilize STMN2 activity and/or function in a target cell (for example, a motor neuron) after administering a STMN2 oligonucleotide e.g. after 1 day, 2 days, 1 week, 2 weeks, 3 weeks, 4 weeks, 5 weeks, 6 weeks, 7 weeks, 8 weeks, 1 month, 2 months, 3, months, 4 months, 5, months, or 6 months or more. In some embodiments, administering such a STMN2 oligonucleotide may be on, e.g., at least a daily basis. The STMN2 oligonucleotide may be administered orally. In some embodiments, the STMN2 oligonucleotide is administered intrathecally, intrathalamically, intracerebroventricularly, or intracisternally. For example, in an embodiment described herein, a STMN2 oligonucleotide is administered intrathecally, intrathalamically or intracisternally about every 3 months. The delay or amelioration of clinical manifestation of a neurological disease in a patient as a consequence of administering a STMN2 oligonucleotide disclosed here may be at least e.g., 6 months, 1 year, 18 months or even 2 years or more as compared to a patient who is not administered a STMN2 oligonucleotide, such as one disclosed herein.

STMN2 oligonucleotides can be used alone or in combination with each other whereby at least two STMN2 oligonucleotides are used together in a single composition or as part of a treatment regimen. STMN2 oligonucleotides may also be used in combination with other drugs or AON for treating neurological diseases or conditions.

In various embodiments, disclosed herein is a method for treating amyotrophic lateral sclerosis (ALS) in a subject in need thereof, the method comprising administering to the subject an oligonucleotide comprising a segment with at most 7 linked nucleosides, and wherein oligonucleotide shares at least 85% identity with any one of SEQ ID NOs: 1-466, SEQ ID NO: 893-1338, SEQ ID NOs: 1342-1366, SEQ ID NOs: 1392-1664, and SEQ ID NOs: 10655-10669, or a pharmaceutically acceptable salt thereof; wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or wherein at least one (i.e., one or more) nucleoside is substituted with a component selected from the group consisting of a 2′-O-(2-methoxyethyl) nucleoside, a 2′-O-methyl nucleoside, a 2′-O—(N-methylacetamide) nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA), optionally wherein the oligonucleotide further comprises a spacer.

In various embodiments, disclosed herein is a method for treating frontotemporal dementia (FTD) in a subject in need thereof, the method comprising administering to the subject an oligonucleotide comprising a segment with at most 7 linked nucleosides, and wherein oligonucleotide shares at least 85% identity with any one of SEQ ID NOs: 1-466, SEQ ID NO: 893-1338, SEQ ID NOs: 1342-1366, SEQ ID NOs: 1392-1664, and SEQ ID NOs: 10655-10669, or a pharmaceutically acceptable salt thereof; wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or wherein at least one (i.e., one or more) nucleoside is substituted with a component selected from the group consisting of a 2′-O-(2-methoxyethyl) nucleoside, a 2′-O-methyl nucleoside, a 2′-O—(N-methylacetamide) nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA), optionally wherein the oligonucleotide further comprises a spacer.

In various embodiments, disclosed herein is a method for treating amyotrophic lateral sclerosis (ALS) with frontotemporal dementia (FTD) in a subject in need thereof, the method comprising administering to the subject an oligonucleotide comprising a segment with at most 7 linked nucleosides, and wherein oligonucleotide shares at least 85% identity with any one of SEQ ID NOs: 1-466, SEQ ID NO: 893-1338, SEQ ID NOs: 1342-1366, SEQ ID NOs: 1392-1664, and SEQ ID NOs: 10655-10669, or a pharmaceutically acceptable salt thereof; wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or wherein at least one (i.e., one or more) nucleoside is substituted with a component selected from the group consisting of a 2′-O-(2-methoxyethyl) nucleoside, a 2′-O-methyl nucleoside, a 2′-O—(N-methylacetamide) nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA), optionally wherein the oligonucleotide further comprises a spacer.

### Methods of Treatment—KCNQ2

The disclosure contemplates, in part, treating neurological diseases including any of amyotrophic lateral sclerosis (ALS), frontotemporal dementia (FTD), Alzheimer's disease (AD), Parkinson's disease (PD), Huntington's disease, progressive supranuclear palsy (PSP), brain trauma, spinal cord injury, corticobasal degeneration (CBD), and Limbic-predominant age-related TDP-43 encephalopathy (LATE) in a patient in need thereof comprising administering a KCNQ2 AON. In some embodiments, provided herein are methods for treatment of a neurological disease in a patient in need thereof, comprising administering a disclosed KCNQ2 AON. In some embodiments of the disclosure, an effective amount of a disclosed KCNQ2 oligonucleotide may be administered to a patient in need thereof to treat a neurological disease, and/or to increase, restore, or stabilize expression of KCNQ2 mRNA that is capable of translation to produce a functional KCNQ2 protein, thereby increase, restore, or stabilize KCNQ2 activity and/or function.

In some embodiments, treating a neurological disease comprises at least ameliorating or reducing one symptom associated with the neurological disease (for example, reducing muscle weakness in a patient with ALS). Methods of treating a neurological disease (for example, ALS, FTD, or ALS with FTD) in a patient suffering therefrom are provided, that include administering a disclosed KCNQ2 AON. In some embodiments, methods of slowing the progression of a neurological disease, for example, a motor neuron disease, are provided.

Provided herein are methods of treating, reducing the risk of developing, or delaying the onset of a neurological disease in a subject in need thereof comprising administering a disclosed KCNQ2 AON. The methods include for example, treating a subject at risk of developing a neurological disease; e.g., administering to the subject an effective amount of a disclosed KCNQ2 AON. Neurological diseases that can be treated in this manner include motor neuron diseases, ALS, FTD, ALS with FTD, progressive bulbar palsy, pseudobulbar palsy, progressive muscular atrophy, primary lateral sclerosis, spinal muscular atrophy, and post-polio syndrome.

Methods of preventing or treating neurological diseases (for example, PD, ALS, FTD, and ALS with FTD) form part of this disclosure. Such methods may comprise administering to a patient in need thereof or a patient at risk, a pharmaceutical preparation comprising a KCNQ2 AON disclosed herein. For example, a method of preventing or treating a neurological disease is provided comprising administering to a patient in need thereof a KCNQ2 AON disclosed herein.

Patients treated using an above method may experience an increase, restoration of, or stabilization of KCNQ2 mRNA expression, which is capable of translation to produce a functional KCNQ2 protein, of at least about 5%, 10%, 20%, 30%, 40% or even 50%, thereby increase, restore, or stabilize KCNQ2 activity and/or function in a target cell (for example, a motor neuron) after administering a KCNQ2 oligonucleotide e.g. after 1 day, 2 days, 1 week, 2 weeks, 3 weeks, 4 weeks, 5 weeks, 6 weeks, 7 weeks, 8 weeks, 1 month, 2 months, 3, months, 4 months, 5, months, or 6 months or more. In some embodiments, administering such a KCNQ2 oligonucleotide may be on, e.g., at least a daily basis. The KCNQ2 oligonucleotide may be administered orally. In some embodiments, the KCNQ2 oligonucleotide is administered intrathecally, intrathalamically, or intracisternally. For example, in an embodiment described herein, a KCNQ2 oligonucleotide is administered intrathecally, intrathalamically or intracisternally about every 3 months. The delay or amelioration of clinical manifestation of a neurological disease in a patient as a consequence of administering a KCNQ2 oligonucleotide disclosed here may be at least e.g., 6 months, 1 year, 18 months or even 2 years or more as compared to a patient who is not administered a KCNQ2 oligonucleotide, such as one disclosed herein.

KCNQ2 oligonucleotides can be used alone or in combination with each other whereby at least two KCNQ2 oligonucleotides are used together in a single composition or as part of a treatment regimen. KCNQ2 oligonucleotides may also be used in combination with other drugs or AON for treating neurological diseases or conditions.

In various embodiments, disclosed herein is a method for treating amyotrophic lateral sclerosis (ALS) in a subject in need thereof, the method comprising administering to the subject an oligonucleotide comprising a segment with at most 7 linked nucleosides, and wherein oligonucleotide shares at least 85% identity with any one of SEQ ID NOs: 1676-1851, SEQ ID NOs: 2028-2529, SEQ ID NOs: 3046-3221, SEQ ID NOs: 3398-3899, or SEQ ID NOs: 4402-4409, or a pharmaceutically acceptable salt thereof; wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or wherein at least one (i.e., one or more) nucleoside is substituted with a component selected from the group consisting of a 2′-O-(2-methoxyethyl) nucleoside, a 2′-O-methyl nucleoside, a 2′-O—(N-methylacetamide) nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA), optionally wherein the oligonucleotide further comprises a spacer.

In various embodiments, disclosed herein is a method for treating frontotemporal dementia (FTD) in a subject in need thereof, the method comprising administering to the subject an oligonucleotide comprising a segment with at most 7 linked nucleosides, and wherein oligonucleotide shares at least 85% identity with any one of SEQ ID NOs: 1676-1851, SEQ ID NOs: 2028-2529, SEQ ID NOs: 3046-3221, SEQ ID NOs: 3398-3899, or SEQ ID NOs: 4402-4409, or a pharmaceutically acceptable salt thereof; wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or wherein at least one (i.e., one or more) nucleoside is substituted with a component selected from the group consisting of a 2′-O-(2-methoxyethyl) nucleoside, a 2′-O-methyl nucleoside, a 2′-O—(N-methylacetamide) nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA), optionally wherein the oligonucleotide further comprises a spacer.

In various embodiments, disclosed herein is a method for treating amyotrophic lateral sclerosis (ALS) with frontotemporal dementia (FTD) in a subject in need thereof, the method comprising administering to the subject an oligonucleotide comprising a segment with at most 7 linked nucleosides, and wherein oligonucleotide shares at least 85% identity with any one SEQ ID NOs: 1676-1851, SEQ ID NOs: 2028-2529, SEQ ID NOs: 3046-3221, SEQ ID NOs: 3398-3899, or SEQ ID NOs: 4402-4409, or a pharmaceutically acceptable salt thereof; wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or wherein at least one (i.e., one or more) nucleoside is substituted with a component selected from the group consisting of a 2′-O-(2-methoxyethyl) nucleoside, a 2′-O-methyl nucleoside, a 2′-O—(N-methylacetamide) nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA), optionally wherein the oligonucleotide further comprises a spacer.

### Methods of Treatment—UNC13A

The disclosure contemplates, in part, treating neurological diseases including any of amyotrophic lateral sclerosis (ALS), frontotemporal dementia (FTD), Alzheimer's disease (AD), Parkinson's disease (PD), Huntington's disease, progressive supranuclear palsy (PSP), brain trauma, spinal cord injury, corticobasal degeneration (CBD), Limbic-predominant age-related TDP-43 encephalopathy (LATE), Cerebral Age-Related TDP-43 With Sclerosis (CARTS), facial onset sensory and motor neuronopathy, Guam Parkinson-dementia complex, multisystem proteinopathy, CTE, and synaptic diseases like autism) in a patient in need thereof comprising administering a UNC13A AON. In some embodiments, provided herein are methods for treatment of a neurological disease in a patient in need thereof, comprising administering a disclosed UNC13A AON. In some embodiments of the disclosure, an effective amount of a disclosed UNC13A oligonucleotide may be administered to a patient in need thereof to treat a neurological disease, and/or to increase, restore, or stabilize expression of UNC13A mRNA that is capable of translation to produce a functional UNC13A protein, thereby increase, restore, or stabilize UNC13A activity and/or function.

In some embodiments, treating a neurological disease comprises at least ameliorating or reducing one symptom associated with the neurological disease (for example, reducing muscle weakness in a patient with ALS). Methods of treating a neurological disease (for example, ALS, FTD, or ALS with FTD) in a patient suffering therefrom are provided, that include administering a disclosed UNC13A AON. In some embodiments, methods of slowing the progression of a neurological disease, for example, a motor neuron disease, are provided.

Provided herein are methods of treating, reducing the risk of developing, or delaying the onset of a neurological disease in a subject in need thereof comprising administering a disclosed UNC13A AON. The methods include for example, treating a subject at risk of developing a neurological disease; e.g., administering to the subject an effective amount of a disclosed UNC13A AON. Neurological diseases that can be treated in this manner include motor neuron diseases, ALS, FTD, ALS with FTD, progressive bulbar palsy, pseudobulbar palsy, progressive muscular atrophy, primary lateral sclerosis, spinal muscular atrophy, and post-polio syndrome.

Methods of preventing or treating neurological diseases (for example, PD, ALS, FTD, and ALS with FTD) form part of this disclosure. Such methods may comprise administering to a patient in need thereof or a patient at risk, a pharmaceutical preparation comprising a UNC13A AON disclosed herein. For example, a method of preventing or treating a neurological disease is provided comprising administering to a patient in need thereof a UNC13A AON disclosed herein.

Patients treated using an above method may experience an increase, restoration of, or stabilization of UNC13A mRNA expression, which is capable of translation to produce a functional UNC13A protein, of at least about 5%, 10%, 20%, 30%, 40% or even 50%, thereby increase, restore, or stabilize UNC13A activity and/or function in a target cell (for example, a motor neuron) after administering a UNC13A oligonucleotide e.g. after 1 day, 2 days, 1 week, 2 weeks, 3 weeks, 4 weeks, 5 weeks, 6 weeks, 7 weeks, 8 weeks, 1 month, 2 months, 3, months, 4 months, 5, months, or 6 months or more. In some embodiments, administering such a UNC13A oligonucleotide may be on, e.g., at least a daily basis. The UNC13A oligonucleotide may be administered orally. In some embodiments, the UNC13A oligonucleotide is administered intrathecally, intrathalamically, intracerebroventricularly, or intracisternally. For example, in an embodiment described herein, a UNC13A oligonucleotide is administered intrathecally, intrathalamically or intracisternally about every 3 months. The delay or amelioration of clinical manifestation of a neurological disease in a patient as a consequence of administering a UNC13A oligonucleotide disclosed here may be at least e.g., 6 months, 1 year, 18 months or even 2 years or more as compared to a patient who is not administered a UNC13A oligonucleotide, such as one disclosed herein.

UNC13A oligonucleotides can be used alone or in combination with each other whereby at least two UNC13A oligonucleotides are used together in a single composition or as part of a treatment regimen. UNC13A oligonucleotides may also be used in combination with other drugs or AON for treating neurological diseases or conditions.

In various embodiments, disclosed herein is a method for treating amyotrophic lateral sclerosis (ALS) in a subject in need thereof, the method comprising administering to the subject an oligonucleotide comprising a segment with at most 7 linked nucleosides, and wherein oligonucleotide shares at least 85% identity with any one of SEQ ID NOs: 4531-5794, SEQ ID NO: 7059-8322, SEQ ID NOs: 9596-9696, or SEQ ID NOs: 10670-10779, or a pharmaceutically acceptable salt thereof; wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or wherein at least one (i.e., one or more) nucleoside is substituted with a component selected from the group consisting of a 2′-O-(2-methoxyethyl) nucleoside, a 2′-O-methyl nucleoside, a 2′-O—(N-methylacetamide) nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA), optionally wherein the oligonucleotide further comprises a spacer.

In various embodiments, disclosed herein is a method for treating frontotemporal dementia (FTD) in a subject in need thereof, the method comprising administering to the subject an oligonucleotide comprising a segment with at most 7 linked nucleosides, and wherein oligonucleotide shares at least 85% identity with any one of SEQ ID NOs: 4531-5794, SEQ ID NO: 7059-8322, SEQ ID NOs: 9596-9696, or SEQ ID NOs: 10670-10779, or a pharmaceutically acceptable salt thereof; wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or wherein at least one (i.e., one or more) nucleoside is substituted with a component selected from the group consisting of a 2′-O-(2-methoxyethyl) nucleoside, a 2′-O-methyl nucleoside, a 2′-O—(N-methylacetamide) nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA), optionally wherein the oligonucleotide further comprises a spacer.

In various embodiments, disclosed herein is a method for treating amyotrophic lateral sclerosis (ALS) with frontotemporal dementia (FTD) in a subject in need thereof, the method comprising administering to the subject an oligonucleotide comprising a segment with at most 7 linked nucleosides, and wherein oligonucleotide shares at least 85% identity with any one of SEQ ID NOs: 4531-5794, SEQ ID NO: 7059-8322, SEQ ID NOs: 9596-9696, or SEQ ID NOs: 10670-10779, or a pharmaceutically acceptable salt thereof; wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or wherein at least one (i.e., one or more) nucleoside is substituted with a component selected from the group consisting of a 2′-O-(2-methoxyethyl) nucleoside, a 2′-O-methyl nucleoside, a 2′-O—(N-methylacetamide) nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA), optionally wherein the oligonucleotide further comprises a spacer.

### Methods of Treatment—SMN2

The disclosure contemplates, in part, treating neurological diseases including spinal muscular atrophy (SMA) in a patient in need thereof comprising administering a SMN2 AON. In some embodiments, provided herein are methods for treatment of a neurological disease in a patient in need thereof, comprising administering a disclosed SMN2 AON. In some embodiments of the disclosure, an effective amount of a disclosed SMN2 oligonucleotide may be administered to a patient in need thereof to treat a neurological disease, and/or to increase, restore, or stabilize expression of SMN2 mRNA that is capable of translation to produce a functional SMN2 protein, thereby increase, restore, or stabilize SMN2 activity and/or function.

In some embodiments, treating a neurological disease comprises at least ameliorating or reducing one symptom associated with the neurological disease (for example, reducing muscle weakness in a patient with SMA). Methods of treating a neurological disease (for example, SMA) in a patient suffering therefrom are provided, that include administering a disclosed SMN2 AON. In some embodiments, methods of slowing the progression of a neurological disease, for example, a motor neuron disease, are provided.

Provided herein are methods of treating, reducing the risk of developing, or delaying the onset of a neurological disease in a subject in need thereof comprising administering a disclosed SMN2 AON. The methods include for example, treating a subject at risk of developing a neurological disease; e.g., administering to the subject an effective amount of a disclosed SMN2 AON. Neurological diseases that can be treated in this manner include spinal muscular atrophy (SMA).

Methods of preventing or treating neurological diseases (for example, SMA) form part of this disclosure. Such methods may comprise administering to a patient in need thereof or a patient at risk, a pharmaceutical preparation comprising a SMN2 AON disclosed herein. For example, a method of preventing or treating a neurological disease is provided comprising administering to a patient in need thereof a SMN2 AON disclosed herein.

Patients treated using an above method may experience an increase, restoration of, or stabilization of SMN2 mRNA expression, which is capable of translation to produce a functional SMN2 protein, of at least about 5%, 10%, 20%, 30%, 40% or even 50%, thereby increase, restore, or stabilize SMN2 activity and/or function in a target cell (for example, a motor neuron) after administering a SMN2 oligonucleotide e.g. after 1 day, 2 days, 1 week, 2 weeks, 3 weeks, 4 weeks, 5 weeks, 6 weeks, 7 weeks, 8 weeks, 1 month, 2 months, 3, months, 4 months, 5, months, or 6 months or more. In some embodiments, administering such a SMN2 oligonucleotide may be on, e.g., at least a daily basis. The SMN2 oligonucleotide may be administered orally. In some embodiments, the SMN2 oligonucleotide is administered intrathecally, intrathalamically, or intracisternally. For example, in an embodiment described herein, a SMN2 oligonucleotide is administered intrathecally, intrathalamically or intracisternally about every 3 months. The delay or amelioration of clinical manifestation of a neurological disease in a patient as a consequence of administering a SMN2 oligonucleotide disclosed here may be at least e.g., 6 months, 1 year, 18 months or even 2 years or more as compared to a patient who is not administered a SMN2 oligonucleotide, such as one disclosed herein.

SMN2 oligonucleotides can be used alone or in combination with each other whereby at least two SMN2 oligonucleotides are used together in a single composition or as part of a treatment regimen. SMN2 oligonucleotides may also be used in combination with other drugs or AON for treating neurological diseases or conditions.

In various embodiments, disclosed herein is a method for treating amyotrophic lateral sclerosis (ALS) in a subject in need thereof, the method comprising administering to the subject an oligonucleotide comprising a segment with at most 7 linked nucleosides, and wherein oligonucleotide shares at least 85% identity with any one of SEQ ID NOs: 9710-10141, SEQ ID NOs: 10574-10651, and SEQ ID NOs: 10783-10808, or a pharmaceutically acceptable salt thereof; wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or wherein at least one (i.e., one or more) nucleoside is substituted with a component selected from the group consisting of a 2′-O-(2-methoxyethyl) nucleoside, a 2′-O-methyl nucleoside, a 2′-O—(N-methylacetamide) nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA), optionally wherein the oligonucleotide further comprises a spacer.

### Treatment and Evaluation

A patient, as described herein, refers to any animal at risk for, suffering from or diagnosed with a neurological disease, including, but not limited to, mammals, primates, and humans. In certain embodiments, the patient may be a non-human mammal such as, for example, a cat, a dog, or a horse. A patient may be an individual diagnosed with a high risk of developing a neurological disease, someone who has been diagnosed with a neurological disease, someone who previously suffered from a neurological disease, or an individual evaluated for symptoms or indications of a neurological disease, for example, any of the signs or symptoms associated with neurological diseases such as: amyotrophic lateral sclerosis (ALS), frontotemporal dementia (FTD), ALS with FTD, Alzheimer's disease (AD), Parkinson's disease (PD), Huntington's disease, progressive supranuclear palsy (PSP), brain trauma, spinal cord injury, corticobasal degeneration (CBD), nerve injuries (e.g., brachial plexus injuries), neuropathies (e.g., chemotherapy induced neuropathy), TDP43 proteinopathies (e.g., chronic traumatic encephalopathy, Perry Syndrome, Dementia with Lewy body in association with Alzheimer's disease, Parkinson's disease with or without dementia, and Limbic-predominant age-related TDP-43 encephalopathy (LATE)), Cerebral Age-Related TDP-43 With Sclerosis (CARTS), facial onset sensory and motor neuronopathy, Guam Parkinson-dementia complex, multisystem proteinopathy, CTE, synaptic diseases like autism and spinal muscular atrophy (SMA).

“A patient in need,” as used herein, refers to a patient suffering from any of the symptoms or manifestations of a neurological disease, a patient who may suffer from any of the symptoms or manifestations of a neurological disease, or any patient who might benefit from a method of the disclosure for treating a neurological disease. A patient in need may include a patient who is diagnosed with a risk of developing a neurological disease, a patient who has suffered from a neurological disease in the past, or a patient who has previously been treated for a neurological disease.

“Effective amount,” as used herein, refers to the amount of an agent that is sufficient to at least partially treat a condition when administered to a patient. The therapeutically effective amount will vary depending on the severity of the condition, the route of administration of the component, and the age, weight, etc. of the patient being treated. Accordingly, an effective amount of a disclosed STMN2 oligonucleotide is the amount of the STMN2 oligonucleotide necessary to treat a neurological disease in a patient such that administration of the agent prevents a neurological disease from occurring in a subject, prevents neurological disease progression (e.g., prevents the onset or increased severity of symptoms of the neurological such as muscle weakening, spasms, or fasciculation), or relieves or completely ameliorates all associated symptoms of a neurological disease, i.e. causes regression of the disease. Accordingly, an effective amount of a disclosed KCNQ2 oligonucleotide is the amount of the KCNQ2 oligonucleotide necessary to treat a neurological disease in a patient such that administration of the agent prevents a neurological disease from occurring in a subject, prevents neurological disease progression (e.g., prevents the onset or increased severity of symptoms of the neurological such as muscle weakening, spasms, or fasciculation), or relieves or completely ameliorates all associated symptoms of a neurological disease, i.e. causes regression of the disease. Accordingly, an effective amount of a disclosed UNC13A oligonucleotide is the amount of the UNC13A oligonucleotide necessary to treat a neurological disease in a patient such that administration of the agent prevents a neurological disease from occurring in a subject, prevents neurological disease progression (e.g., prevents the onset or increased severity of symptoms of the neurological such as muscle weakening, spasms, or fasciculation), or relieves or completely ameliorates all associated symptoms of a neurological disease, i.e. causes regression of the disease. Accordingly, an effective amount of a disclosed SMN2 oligonucleotide is the amount of the SMN2 oligonucleotide necessary to treat a neurological disease in a patient such that administration of the agent prevents a neurological disease from occurring in a subject, prevents neurological disease progression (e.g., prevents the onset or increased severity of symptoms of the neurological such as muscle weakening, spasms, or fasciculation), or relieves or completely ameliorates all associated symptoms of a neurological disease, i.e. causes regression of the disease.

Efficacy of treatment may be evaluated by means of evaluation of gross symptoms associated with a neurological disease, analysis of tissue histology, biochemical assay, imaging methods such as, for example, magnetic resonance imaging, or other known methods. For instance, efficacy of treatment may be evaluated by analyzing gross symptoms of the disease such as changes in muscle strength and control or other aspects of gross pathology associated with a neurological disease following administration, to a patient suffering from a neurological disease, a disclosed STMN2 oligonucleotide. For instance, efficacy of treatment may be evaluated by analyzing gross symptoms of the disease such as changes in muscle strength and control or other aspects of gross pathology associated with a neurological disease following administration, to a patient suffering from a neurological disease, a disclosed KCNQ2 oligonucleotide. For instance, efficacy of treatment may be evaluated by analyzing gross symptoms of the disease such as changes in muscle strength and control or other aspects of gross pathology associated with a neurological disease following administration, to a patient suffering from a neurological disease, a disclosed UNC13A oligonucleotide. For instance, efficacy of treatment may be evaluated by analyzing gross symptoms of the disease such as changes in muscle strength and control or other aspects of gross pathology associated with a neurological disease following administration, to a patient suffering from a neurological disease, a disclosed SMN2 oligonucleotide.

Efficacy of treatment may also be evaluated at the tissue or cellular level, for example, by means of obtaining a tissue biopsy (e.g., a brain, spinal, muscle, motor neuron tissue biopsy, or olfactory neurosphere cell biopsy) and evaluating gross tissue or cell morphology or staining properties. Biochemical assays that examine protein or RNA expression may also be used to evaluate efficacy of treatment. For instance, one may evaluate levels of a protein or gene product indicative of a neurological disease, in dissociated cells or non-dissociated tissue via immunocytochemical, immunohistochemical, Western blotting, or Northern blotting methods, or methods useful for evaluating RNA levels such as quantitative or semi-quantitative polymerase chain (e.g., digital PCR (DigitalPCR, dPCR, or ddPCR), qPCR etc.) reaction. One may also evaluate the presence or level of expression of useful biomarkers (e.g., neurofilament light (NEFL), neurofilament heavy (NEFH), TDP-43 or p75 extracellular domain (p75^{ECD})) found in spinal cord fluid, cerebrospinal fluid, extracellular vesicles (for example, exosome-like cerebrospinal fluid extracellular vesicles (“CSF exosomes”), such as those described in Welton et al., (2017) “Cerebrospinal fluid extracellular vesicle enrichment for protein biomarker discovery in neurological disease; multiple sclerosis” J Extracell Vesicles., 6(1):1-10; and Street et al., (2012) “Identification and proteomic profiling of exosomes in human cerebrospinal fluid” J Transl. Med., 10:5), urine, fecal matter, lymphatic fluid, blood, plasma, or serum to evaluate disease state and efficacy of treatment. One may also evaluate the presence or level of expression of useful biomarkers found in the plasma, neuronal extracellular vesicles/exosomes. Additional measurements of efficacy may include strength duration time constant (SDTC), short interval cortical inhibition (SICI), dynamometry, accurate test of limb isometric strength (ATLIS), compound muscle action potential (CMAP), and ALSFRS-R. In certain embodiments, urinary neurotrophin receptor p75 extracellular domain (p75^{ECD}) is a disease progression and prognostic biomarker in amyotrophic lateral sclerosis (ALS). Phosphorylated neurofilament heavy chain (pNFH) in cerebrospinal fluid (CSF) predict disease status and survival in C9ORF72-associated amyotrophic lateral sclerosis (c9ALS) patients. CSF pNFH as a prognostic biomarker for clinical trials, which will increase the likelihood of successfully developing a treatment for c9ALS.

### STMN2

In evaluating efficacy of treatment, suitable controls may be chosen to ensure a valid assessment. For instance, one can compare symptoms evaluated in a patient with a neurological disease following administration of a disclosed STMN2 oligonucleotide to those symptoms in the same patient prior to treatment or at an earlier point in the course of treatment or in another patient not diagnosed with the neurological disease. Alternatively, one may compare the results of biochemical or histological analysis of tissue following administration of a disclosed STMN2 oligonucleotide with those of tissue from the same patient or from an individual not diagnosed with the neurological disease or from the same patient prior to administration of the STMN2 oligonucleotide. Additionally, one may compare blood, plasma, serum, cell, urine, lymphatic fluid, spinal cord fluid, cerebrospinal fluid, or fecal samples following administration of the STMN2 oligonucleotide with comparable samples from an individual not diagnosed with the neurological disease or from the same patient prior to administration of the STMN2 oligonucleotide. In some embodiments one may compare extracellular vesicles (for example CSF exosomes), following administration of the STMN2 oligonucleotide with extracellular vesicles from an individual not diagnosed with the neurological disease or from the same patient prior to administration of the STMN2 oligonucleotide.

Validation of STMN2 oligonucleotides may be determined by direct or indirect assessment of STMN2 expression levels or activity. For instance, biochemical assays that measure STMN2 protein or RNA expression may be used to evaluate overall effect on STMN2 transcripts (for example, a STMN2 pre-mRNA comprising a cryptic exon) comprising a sequence that shares at least 90% (e.g., 90%, 91%, 92%, 93%, 94%, 95%, 96%, 97%, 98%, 99%, or 100%) identity to SEQ ID NO: 1339 or SEQ ID NO: 1341. For instance, one may measure STMN2 protein levels in cells or tissue by Western blot to evaluate overall STMN2 levels. One may also measure STMN2 mRNA levels by means of Northern blot or quantitative polymerase chain reaction to determine overall effect on STMN2 transcripts (for example, a STMN2 pre-mRNA comprising a cryptic exon) comprising a sequence that shares at least 90% (e.g., 90%, 91%, 92%, 93%, 94%, 95%, 96%, 97%, 98%, 99%, or 100%) identity to SEQ ID NO: 1339 or SEQ ID NO: 1341. One may also evaluate STMN2 protein levels or levels of another protein indicative of STMN2 signaling activity in dissociated cells, non-dissociated tissue, extracellular vesicles (for example, CSF exosomes), blood, serum, or fecal matter via immunocytochemical or immunohistochemical methods.

Modulation of expression levels of STMN2 transcripts (for example, a STMN2 pre-mRNA comprising a cryptic exon) comprising a sequence that shares at least 90% (e.g., 90%, 91%, 92%, 93%, 94%, 95%, 96%, 97%, 98%, 99%, or 100%) identity to SEQ ID NO: 1339 or SEQ ID NO: 1341 may also be evaluated indirectly by measuring parameters such as autophagy, endocytosis, protein aggregation, and the presence or level of expression of useful biomarkers (e.g., neurofilament light (NEFL), neurofilament heavy (NEFH), TDP-43, or p75^{EC}D found in plasma, spinal cord fluid, cerebrospinal fluid, extracellular vesicles (for example, CSF exosomes), blood, urine, lymphatic fluid, fecal matter, or tissue to evaluate the modulation of expression of STMN2 transcripts (for example, a STMN2 pre-mRNA comprising a cryptic exon) comprising a sequence that shares at least 90% (e.g., 90%, 91%, 92%, 93%, 94%, 95%, 96%, 97%, 98%, 99%, or 100%) identity to SEQ ID NO: 1339 or SEQ ID NO: 1341. Modulation of expression levels of STMN2 transcripts (for example, a STMN2 pre-mRNA comprising a cryptic exon) comprising a sequence that shares at least 90% (e.g., 90%, 91%, 92%, 93%, 94%, 95%, 96%, 97%, 98%, 99%, or 100%) identity to SEQ ID NO: 1339 or SEQ ID NO: 1341 may also be evaluated indirectly by measuring parameters such as autophagy, endocytosis, protein aggregation, and the presence or level of expression of physiological biomarkers such as compound muscle action potential (CMAP). Additional measurements may include strength duration time constant (SDTC), short interval cortical inhibition (SICI), pyrrolidinyl, accurate test of limb isometric strength (ATLIS), compound muscle action potential, and ALSFRS-R. In certain embodiments, urinary neurotrophin receptor p75 extracellular domain (p75^{ECD}) is a disease progression and prognostic biomarker in amyotrophic lateral sclerosis (ALS). Phosphorylated neurofilament heavy chain (pNFH) in cerebrospinal fluid (CSF) predict disease status and survival in c9ALS patients. CSF pNFH as a prognostic biomarker for clinical trials, which will increase the likelihood of successfully developing a treatment for c9ALS.

The disclosure also provides methods of restoring expression of full length STMN2 transcripts in cells of a patient suffering from a neurological disease. Full length STMN2 transcripts may be restored in any cell in which STMN2 expression or activity occurs, including cells of the nervous system (including the central nervous system (e.g., spinal cord or brain), the peripheral nervous system, motor neurons, glial cells, astrocytes, oligodendrocytes, microglia, the brain, the brain stem, the frontal lobes, the temporal lobes, the spinal cord), the musculoskeletal system, spinal fluid, and cerebrospinal fluid. Cells of the musculoskeletal system include skeletal muscle cells (e.g., myocytes). Motor neurons include upper motor neurons and lower motor neurons.

### KCNQ2

In evaluating efficacy of treatment, suitable controls may be chosen to ensure a valid assessment. For instance, one can compare symptoms evaluated in a patient with a neurological disease following administration of a disclosed KCNQ2 oligonucleotide to those symptoms in the same patient prior to treatment or at an earlier point in the course of treatment or in another patient not diagnosed with the neurological disease. Alternatively, one may compare the results of biochemical or histological analysis of tissue following administration of a disclosed KCNQ2 oligonucleotide with those of tissue from the same patient or from an individual not diagnosed with the neurological disease or from the same patient prior to administration of the KCNQ2 oligonucleotide. Additionally, one may compare blood, plasma, serum, cell, urine, lymphatic fluid, spinal cord fluid, cerebrospinal fluid, or fecal samples following administration of the KCNQ2 oligonucleotide with comparable samples from an individual not diagnosed with the neurological disease or from the same patient prior to administration of the KCNQ2 oligonucleotide. In some embodiments one may compare extracellular vesicles (for example CSF exosomes), following administration of the KCNQ2 oligonucleotide with extracellular vesicles from an individual not diagnosed with the neurological disease or from the same patient prior to administration of the KCNQ2 oligonucleotide.

Validation of KCNQ2 oligonucleotides may be determined by direct or indirect assessment of KCNQ2 expression levels or activity. For instance, biochemical assays that measure KCNQ2 protein or RNA expression may be used to evaluate overall effect on KCNQ2 transcripts) comprising a sequence that shares at least 90% (e.g., 90%, 91%, 92%, 93%, 94%, 95%, 96%, 97%, 98%, 99%, or 100%) identity to any one of SEQ ID NOs: 3032-3043. For instance, one may measure KCNQ2 protein levels in cells or tissue by Western blot to evaluate overall KCNQ2 levels. One may also measure KCNQ2 mRNA levels by means of Northern blot or quantitative polymerase chain reaction to determine overall effect on KCNQ2 transcripts (for example, a KCNQ2 pre-mRNA) comprising a sequence that shares at least 90% (e.g., 90%, 91%, 92%, 93%, 94%, 95%, 96%, 97%, 98%, 99%, or 100%) identity to any one of SEQ ID NOs: 3032-3043. One may also evaluate KCNQ2 protein levels or levels of another protein indicative of KCNQ2 signaling activity in dissociated cells, non-dissociated tissue, extracellular vesicles (for example, CSF exosomes), blood, serum, or fecal matter via immunocytochemical or immunohistochemical methods.

Modulation of expression levels of KCNQ2 transcripts (for example, a KCNQ2 pre-mRNA) comprising a sequence that shares at least 90% (e.g., 90%, 91%, 92%, 93%, 94%, 95%, 96%, 97%, 98%, 99%, or 100%) identity to any one of SEQ ID NOs: 3032-3043 may also be evaluated indirectly by measuring parameters such as autophagy, endocytosis, protein aggregation, and the presence or level of expression of useful biomarkers (e.g., neurofilament light (NEFL), neurofilament heavy (NEFH), TDP-43, or p75^{ECD} found in plasma, spinal cord fluid, cerebrospinal fluid, extracellular vesicles (for example, CSF exosomes), blood, urine, lymphatic fluid, fecal matter, or tissue to evaluate the modulation of expression of KCNQ2 transcripts (for example, a KCNQ2 pre-mRNA) comprising a sequence that shares at least 90% (e.g., 90%, 91%, 92%, 93%, 94%, 95%, 96%, 97%, 98%, 99%, or 100%) identity to any one of SEQ ID NOs: 3032-3043. Modulation of expression levels of KCNQ2 transcripts (for example, a KCNQ2 pre-mRNA) comprising a sequence that shares at least 90% (e.g., 90%, 91%, 92%, 93%, 94%, 95%, 96%, 97%, 98%, 99%, or 100%) identity to any one of SEQ ID NOs: 3032-3043 may also be evaluated indirectly by measuring parameters such as autophagy, endocytosis, protein aggregation, and the presence or level of expression of physiological biomarkers such as compound muscle action potential (CMAP). Additional measurements may include strength duration time constant (SDTC), short interval cortical inhibition (SICI), dynamometry, accurate test of limb isometric strength (ATLIS), compound muscle action potential, and ALSFRS-R. In certain embodiments, urinary neurotrophin receptor p75 extracellular domain (p75^{ECD}) is a disease progression and prognostic biomarker in amyotrophic lateral sclerosis (ALS). Phosphorylated neurofilament heavy chain (pNFH) in cerebrospinal fluid (CSF) predict disease status and survival in c9ALS patients. CSF pNFH as a prognostic biomarker for clinical trials, which will increase the likelihood of successfully developing a treatment for c9ALS.

The disclosure also provides methods of restoring expression of full length KCNQ2 transcripts in cells of a patient suffering from a neurological disease. Full length KCNQ2 transcripts may be restored in any cell in which KCNQ2 expression or activity occurs, including cells of the nervous system (including the central nervous system (e.g., spinal cord or brain), the peripheral nervous system, motor neurons, glial cells, astrocytes, oligodendrocytes, microglia, the brain, the brain stem, the frontal lobes, the temporal lobes, the spinal cord), the musculoskeletal system, spinal fluid, and cerebrospinal fluid. Cells of the musculoskeletal system include skeletal muscle cells (e.g., myocytes). Motor neurons include upper motor neurons and lower motor neurons.

### UNC13A

In evaluating efficacy of treatment, suitable controls may be chosen to ensure a valid assessment. For instance, one can compare symptoms evaluated in a patient with a neurological disease following administration of a disclosed UNC13A oligonucleotide to those symptoms in the same patient prior to treatment or at an earlier point in the course of treatment or in another patient not diagnosed with the neurological disease. Alternatively, one may compare the results of biochemical or histological analysis of tissue following administration of a disclosed UNC13A oligonucleotide with those of tissue from the same patient or from an individual not diagnosed with the neurological disease or from the same patient prior to administration of the UNC13A oligonucleotide. Additionally, one may compare blood, plasma, serum, cell, urine, lymphatic fluid, spinal cord fluid, cerebrospinal fluid, or fecal samples following administration of the UNC13A oligonucleotide with comparable samples from an individual not diagnosed with the neurological disease or from the same patient prior to administration of the UNC13A oligonucleotide. In some embodiments one may compare extracellular vesicles (for example CSF exosomes), following administration of the UNC13A oligonucleotide with extracellular vesicles from an individual not diagnosed with the neurological disease or from the same patient prior to administration of the UNC13A oligonucleotide.

Validation of UNC13A oligonucleotides may be determined by direct or indirect assessment of UNC13A expression levels or activity. For instance, biochemical assays that measure UNC13A protein or RNA expression may be used to evaluate overall effect on UNC13A transcripts (for example, a UNC13A pre-mRNA) comprising a sequence that shares at least 90% (e.g., 90%, 91%, 92%, 93%, 94%, 95%, 96%, 97%, 98%, 99%, or 100%) identity to any one of SEQ ID NOs: 5057-5065. For instance, one may measure UNC13A protein levels in cells or tissue by Western blot to evaluate overall UNC13A levels. One may also measure UNC13A mRNA levels by means of Northern blot or quantitative polymerase chain reaction to determine overall effect on UNC13A transcripts (for example, a UNC13A pre-mRNA) comprising a sequence that shares at least 90% (e.g., 90%, 91%, 92%, 93%, 94%, 95%, 96%, 97%, 98%, 99%, or 100%) identity to any one of SEQ ID NOs: 5057-5065. One may also evaluate UNC13A protein levels or levels of another protein indicative of UNC13A signaling activity in dissociated cells, non-dissociated tissue, extracellular vesicles (for example, CSF exosomes), blood, serum, or fecal matter via immunocytochemical or immunohistochemical methods.

Modulation of expression levels of UNC13A transcripts (for example, a UNC13A pre-mRNA) comprising a sequence that shares at least 90% (e.g., 90%, 91%, 92%, 93%, 94%, 95%, 96%, 97%, 98%, 99%, or 100%) identity to any one of SEQ ID NOs: 5057-5065 may also be evaluated indirectly by measuring parameters such as autophagy, endocytosis, protein aggregation, and the presence or level of expression of useful biomarkers (e.g., neurofilament light (NEFL), neurofilament heavy (NEFH), TDP-43, or p75^{ECD} found in plasma, spinal cord fluid, cerebrospinal fluid, extracellular vesicles (for example, CSF exosomes), blood, urine, lymphatic fluid, fecal matter, or tissue to evaluate the modulation of expression of UNC13A transcripts (for example, a UNC13A pre-mRNA) comprising a sequence that shares at least 90% (e.g., 90%, 91%, 92%, 93%, 94%, 95%, 96%, 97%, 98%, 99%, or 100%) identity to any one of SEQ ID NOs: 5057-5065. Modulation of expression levels of UNC13A transcripts (for example, a UNC13A pre-mRNA) comprising a sequence that shares at least 90% (e.g., 90%, 91%, 92%, 93%, 94%, 95%, 96%, 97%, 98%, 99%, or 100%) identity to any one of SEQ ID NOs: 5057-5065 may also be evaluated indirectly by measuring parameters such as autophagy, endocytosis, protein aggregation, and the presence or level of expression of physiological biomarkers such as compound muscle action potential (CMAP). Additional measurements may include strength duration time constant (SDTC), short interval cortical inhibition (SICI), dynamometry, accurate test of limb isometric strength (ATLIS), compound muscle action potential, and ALSFRS-R. In certain embodiments, urinary neurotrophin receptor p75 extracellular domain (p75^{ECD}) is a disease progression and prognostic biomarker in amyotrophic lateral sclerosis (ALS). Phosphorylated neurofilament heavy chain (pNFH) in cerebrospinal fluid (CSF) predict disease status and survival in c9ALS patients. CSF pNFH as a prognostic biomarker for clinical trials, which will increase the likelihood of successfully developing a treatment for c9ALS.

The disclosure also provides methods of restoring expression of full length UNC13A transcripts in cells of a patient suffering from a neurological disease. Full length UNC13A transcripts may be restored in any cell in which UNC13A expression or activity occurs, including cells of the nervous system (including the central nervous system (e.g., spinal cord or brain), the peripheral nervous system, motor neurons, glial cells, astrocytes, oligodendrocytes, microglia, the brain, the brain stem, the frontal lobes, the temporal lobes, the spinal cord), the musculoskeletal system, spinal fluid, and cerebrospinal fluid. Cells of the musculoskeletal system include skeletal muscle cells (e.g., myocytes). Motor neurons include upper motor neurons and lower motor neurons.

### SMN2

In evaluating efficacy of treatment, suitable controls may be chosen to ensure a valid assessment. For instance, one can compare symptoms evaluated in a patient with a neurological disease following administration of a disclosed SMN2 oligonucleotide to those symptoms in the same patient prior to treatment or at an earlier point in the course of treatment or in another patient not diagnosed with the neurological disease. Alternatively, one may compare the results of biochemical or histological analysis of tissue following administration of a disclosed SMN2 oligonucleotide with those of tissue from the same patient or from an individual not diagnosed with the neurological disease or from the same patient prior to administration of the SMN2 oligonucleotide. Additionally, one may compare blood, plasma, serum, cell, urine, lymphatic fluid, spinal cord fluid, cerebrospinal fluid, or fecal samples following administration of the SMN2 oligonucleotide with comparable samples from an individual not diagnosed with the neurological disease or from the same patient prior to administration of the SMN2 oligonucleotide. In some embodiments one may compare extracellular vesicles (for example CSF exosomes), following administration of the SMN2 oligonucleotide with extracellular vesicles from an individual not diagnosed with the neurological disease or from the same patient prior to administration of the SMN2 oligonucleotide.

Validation of SMN2 oligonucleotides may be determined by direct or indirect assessment of SMN2 expression levels or activity. For instance, biochemical assays that measure SMN2 protein or RNA expression may be used to evaluate overall effect on SMN2 transcripts) comprising a sequence that shares at least 90% (e.g., 90%, 91%, 92%, 93%, 94%, 95%, 96%, 97%, 98%, 99%, or 100%) identity to any one of SEQ ID NOs: 9698-9709. For instance, one may measure SMN2 protein levels in cells or tissue by Western blot to evaluate overall SMN2 levels. One may also measure SMN2 mRNA levels by means of Northern blot or quantitative polymerase chain reaction to determine overall effect on SMN2 transcripts (for example, a SMN2 pre-mRNA) comprising a sequence that shares at least 90% (e.g., 90%, 91%, 92%, 93%, 94%, 95%, 96%, 97%, 98%, 99%, or 100%) identity to any one of SEQ ID NOs: 9698-9709. One may also evaluate SMN2 protein levels or levels of another protein indicative of SMN2 signaling activity in dissociated cells, non-dissociated tissue, extracellular vesicles (for example, CSF exosomes), blood, serum, or fecal matter via immunocytochemical or immunohistochemical methods.

Modulation of expression levels of SMN2 transcripts (for example, a SMN2 pre-mRNA) comprising a sequence that shares at least 90% (e.g., 90%, 91%, 92%, 93%, 94%, 95%, 96%, 97%, 98%, 99%, or 100%) identity to any one of SEQ ID NOs: 9698-9709 may also be evaluated indirectly by measuring parameters such as autophagy, endocytosis, protein aggregation, and the presence or level of expression of useful biomarkers (e.g., neurofilament light (NEFL), neurofilament heavy (NEFH), TDP-43, or p75^{ECD} found in plasma, spinal cord fluid, cerebrospinal fluid, extracellular vesicles (for example, CSF exosomes), blood, urine, lymphatic fluid, fecal matter, or tissue to evaluate the modulation of expression of SMN2 transcripts (for example, a SMN2 pre-mRNA) comprising a sequence that shares at least 90% (e.g., 90%, 91%, 92%, 93%, 94%, 95%, 96%, 97%, 98%, 99%, or 100%) identity to any one of SEQ ID NOs: 9698-9707. Modulation of expression levels of SMN2 transcripts (for example, a SMN2 pre-mRNA) comprising a sequence that shares at least 90% (e.g., 90%, 91%, 92%, 93%, 94%, 95%, 96%, 97%, 98%, 99%, or 100%) identity to any one of SEQ ID NOs: 9698-9707 may also be evaluated indirectly by measuring parameters such as autophagy, endocytosis, protein aggregation, and the presence or level of expression of physiological biomarkers such as compound muscle action potential (CMAP). Additional measurements may include strength duration time constant (SDTC), short interval cortical inhibition (SICI), dynamometry, accurate test of limb isometric strength (ATLIS), and compound muscle action potential. In certain embodiments, urinary neurotrophin receptor p75 extracellular domain (p75^{ECD}) is a disease progression and prognostic biomarker in amyotrophic lateral sclerosis (ALS). Phosphorylated neurofilament heavy chain (pNFH) in cerebrospinal fluid (CSF) predict disease status and survival in c9ALS patients. CSF pNFH as a prognostic biomarker for clinical trials, which will increase the likelihood of successfully developing a treatment for c9ALS.

The disclosure also provides methods of restoring expression of full length SMN2 transcripts in cells of a patient suffering from a neurological disease. Full length SMN2 transcripts may be restored in any cell in which SMN2 expression or activity occurs, including cells of the nervous system (including the central nervous system (e.g., spinal cord or brain), the peripheral nervous system, motor neurons, glial cells, astrocytes, oligodendrocytes, microglia, the brain, the brain stem, the frontal lobes, the temporal lobes, the spinal cord), the musculoskeletal system, spinal fluid, and cerebrospinal fluid. Cells of the musculoskeletal system include skeletal muscle cells (e.g., myocytes). Motor neurons include upper motor neurons and lower motor neurons.

### Pharmaceutical Compositions and Routes of Administration

The present disclosure also provides methods for treating a neurological disease via administration of a pharmaceutical composition comprising a disclosed STMN2 oligonucleotide, KCNQ2 oligonucleotide, UNC13A oligonucleotide, and/or SMN2 oligonucleotide. In another aspect, the disclosure provides a pharmaceutical composition for use in treating a neurological disease. The pharmaceutical composition may be comprised of a disclosed STMN2 oligonucleotide, and a pharmaceutically acceptable carrier. As used herein the term “pharmaceutical composition” means, for example, a mixture containing a specified amount of a therapeutic compound, e.g., a therapeutically effective amount, of a therapeutic compound in a pharmaceutically acceptable carrier to be administered to a mammal, e.g., a human, in order to treat a neurological disease. In some embodiments, described herein are pharmaceutical compositions comprising a disclosed STMN2 oligonucleotide, KCNQ2 oligonucleotide, UNC13A oligonucleotide, and/or SMN2 oligonucleotide, and a pharmaceutically acceptable carrier. In another aspect, the disclosure provides use of a disclosed STMN2 oligonucleotide, KCNQ2 oligonucleotide, UNC13A oligonucleotide, and/or SMN2 oligonucleotide in the manufacture of a medicament for treating a neurological disease. “Medicament,” as used herein, has essentially the same meaning as the term “pharmaceutical composition.”

As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art. In one embodiment the pharmaceutical composition is administered orally and includes an enteric coating suitable for regulating the site of absorption of the encapsulated substances within the digestive system or gut. For example, an enteric coating can include an ethylacrylate-methacrylic acid copolymer.

In one embodiment, a disclosed STMN2 oligonucleotide, KCNQ2 oligonucleotide, UNC13A oligonucleotide, and/or SMN2 oligonucleotide, and any pharmaceutical composition thereof may be administered by one or several routes, including topically, intrathecally, intrathalamically, intracerebroventricularly, intracisternally, parenterally, orally, rectally, buccally, sublingually, vaginally, pulmonarily, intratracheally, intranasally, transdermally, or intraduodenally. The term parenteral as used herein includes subcutaneous injections, intrapancreatic administration, intravenous, intracisternal, intracerebroventricular, intrathecal, intrathalamic, intramuscular, intraperitoneal, intrasternal injection or infusion techniques. For example, a disclosed STMN2 oligonucleotide, KCNQ2 oligonucleotide, UNC13A oligonucleotide, and/or SMN2 oligonucleotide may be administered subcutaneously to a subject. In another example, a disclosed STMN2 oligonucleotide, KCNQ2 oligonucleotide, UNC13A oligonucleotide, and/or SMN2 oligonucleotide may be administered orally to a subject. In another example, a disclosed STMN2 oligonucleotide, KCNQ2 oligonucleotide, UNC13A oligonucleotide, and/or SMN2 oligonucleotide may be administered directly to the nervous system, or specific regions or cells of the nervous system (e.g., the brain, brain stem, lower motor neurons, spinal cord, upper motor neurons) via parenteral administration, for example, a disclosed STMN2 oligonucleotide, KCNQ2 oligonucleotide, UNC13A oligonucleotide, and/or SMN2 oligonucleotide may be administered intrathecally, intrathalamically, intracerebroventricularly, or intracisternally.

In various embodiments, a STMN2 oligonucleotide, KCNQ2 oligonucleotide, UNC13A oligonucleotide, and/or SMN2 oligonucleotide, for example a STMN2 AON, KCNQ2 AON, UNC13A AON, and/or SMN2 AON, can be exposed to calcium-containing buffers prior to administration. Such calcium-containing buffers can mitigate toxicity adverse effects of the STMN2 oligonucleotide, KCNQ2 oligonucleotide, UNC13A oligonucleotide, and/or SMN2 oligonucleotide. Further details of exposing an example antisense oligonucleotide to calcium-containing buffers is described in Moazami, et al., Quantifying and Mitigating Motor Phenotypes Induced by Antisense Oligonucleotides in the Central Nervous System, bioRxiv 2021.02.14.431096, which is hereby incorporated by reference in its entirety.

In some embodiments, a STMN2 oligonucleotide, KCNQ2 oligonucleotide, UNC13A oligonucleotide, and/or SMN2 oligonucleotide, for example a STMN2 AON, KCNQ2 AON, UNC13A AON, and/or SMN2 AON, can be encapsulated in a nanoparticle coating. It is believed that nanoparticle encapsulation prevents AON degradation and enhances cellular uptake. For example, in some embodiments a STMN2 oligonucleotide, KCNQ2 oligonucleotide, UNC13A oligonucleotide, and/or SMN2 oligonucleotide is encapsulated in a coating of a cationic polymer, for example, a synthetic polymer (e.g., poly-L-lysine, polyamidoamine, a poly(O-amino ester), and polyethyleneimine) or a naturally occurring polymer (e.g., chitosan and a protamine). In some embodiments, a STMN2 oligonucleotide, KCNQ2 oligonucleotide, UNC13A oligonucleotide, and/or SMN2 oligonucleotide is encapsulated in a lipid or lipid-like material, for example, a cationic lipid, a cationic lipid-like material, or an ionizable lipid that is positively charged only at an acidic pH. For example, in some embodiments, a STMN2 oligonucleotide, KCNQ2 oligonucleotide, UNC13A oligonucleotide, and/or SMN2 oligonucleotide is encapsulated in a lipid nanoparticle that includes hydrophobic moieties, e.g., cholesterol and/or a polyethylene glycol (PEG) lipid.

Pharmaceutical compositions containing a disclosed STMN2 oligonucleotide, KCNQ2 oligonucleotide, UNC13A oligonucleotide, and/or SMN2 oligonucleotide, such as those disclosed herein, can be presented in a dosage unit form and can be prepared by any suitable method. A pharmaceutical composition should be formulated to be compatible with its intended route of administration. Useful formulations can be prepared by methods well known in the pharmaceutical art. For example, see Remington's Pharmaceutical Sciences, 18^{th} ed. (Mack Publishing Company, 1990).

Pharmaceutical formulations, in some embodiments, are sterile. Sterilization can be accomplished, for example, by filtration through sterile filtration membranes. Where the composition is lyophilized, filter sterilization can be conducted prior to or following lyophilization and reconstitution.

### Parenteral Administration

The pharmaceutical compositions of the disclosure can be formulated for parenteral administration, e.g., formulated for injection via the intravenous, intracisternal, intracerebroventricular, intramuscular, subcutaneous, intrathecal, intrathalamic, intralesional, or intraperitoneal routes. The preparation of an aqueous composition, such as an aqueous pharmaceutical composition containing a disclosed STMN2 oligonucleotide, KCNQ2 oligonucleotide, UNC13A oligonucleotide, and/or SMN2 oligonucleotide, will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

The pharmaceutical forms suitable for injectable use include sterile aqueous solutions or dispersions; formulations including normal saline, artificial cerebrospinal fluid, sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

Solutions of active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. In addition, sterile, fixed oils may be employed as a solvent or suspending medium. For this purpose any bland fixed oil can be employed including synthetic mono- or diglycerides. In addition, fatty acids such as oleic acid can be used in the preparation of injectables. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In one embodiment, a disclosed STMN2 antisense oligonucleotide may be suspended in a carrier fluid comprising 1% (w/v) sodium carboxymethylcellulose and 0.1% (v/v) TWEEN™ 80. Under ordinary conditions of storage and use, these preparations contain a preservative to prevent the growth of microorganisms.

Injectable preparations, for example, sterile injectable aqueous or oleaginous suspensions may be formulated according to the known art using suitable dispersing or wetting agents and suspending agents. Generally, dispersions are prepared by incorporating the various sterilized active ingredients into a sterile vehicle which contains the basic dispersion medium and the required other ingredients from those enumerated above. Sterile injectable solutions of the disclosure may be prepared by incorporating a disclosed STMN2 antisense oligonucleotide, KCNQ2 antisense oligonucleotide, UNC13A antisense oligonucleotide, and/or SMN2 antisense oligonucleotide in the required amount of the appropriate solvent with various of the other ingredients enumerated above, as required, followed by filtered sterilization. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The injectable formulations can be sterilized, for example, by filtration through a bacteria-retaining filter.

The preparation of more, or highly concentrated solutions for intramuscular injection is also contemplated. In this regard, the use of DMSO as solvent is preferred as this will result in extremely rapid penetration, delivering high concentrations of the disclosed oligonucleotide to a small area.

Suitable preservatives for use in such a solution include benzalkonium chloride, benzethonium chloride, chlorobutanol, thimerosal and the like. Suitable buffers include boric acid, sodium and potassium bicarbonate, sodium and potassium borates, sodium and potassium carbonate, sodium acetate, sodium biphosphate and the like, in amounts sufficient to maintain the pH at between about pH 6 and pH 8, and for example, between about pH 7 and pH 7.5. Suitable tonicity agents are dextran 40, dextran 70, dextrose, glycerin, potassium chloride, propylene glycol, sodium chloride, and the like, such that the sodium chloride equivalent of the solution is in the range 0.9 plus or minus 0.2%. Suitable antioxidants and stabilizers include sodium bisulfite, sodium metabisulfite, sodium pyrrolidinyl, thiourea and the like. Suitable wetting and clarifying agents include polysorbate 80, polysorbate 20, poloxamer 282 and tyloxapol. Suitable viscosity-increasing agents include dextran 40, dextran 70, gelatin, glycerin, hydroxyethylcellulose, hydroxymethylpropylcellulose, lanolin, methylcellulose, petrolatum, polyethylene glycol, polyvinyl alcohol, polyvinylpyrrolidone, carboxymethylcellulose and the like.

### Oral Administration

In some embodiments, contemplated herein are compositions suitable for oral delivery of a disclosed STMN2 oligonucleotide, KCNQ2 oligonucleotide, UNC13A oligonucleotide, and/or SMN2 oligonucleotide, e.g., tablets that include an enteric coating, e.g., a gastro-resistant coating, such that the compositions may deliver a STMN2 oligonucleotide, KCNQ2 oligonucleotide, UNC13A oligonucleotide, and/or SMN2 oligonucleotide to, e.g., the gastrointestinal tract of a patient.

For example, a tablet for oral administration is provided that comprises granules (e.g., is at least partially formed from granules) that include a disclosed STMN2 oligonucleotide, e.g., a STMN2 oligonucleotide represented by any SEQ ID NOs: 1-466, SEQ ID NO: 893-1338, SEQ ID NOs: 1342-1366, SEQ ID NOs: 1392-1664, and SEQ ID NOs: 10655-10669 that targets a STMN2 transcript comprising a sequence that shares at least 90% (e.g., 90%, 91%, 92%, 93%, 94%, 95%, 96%, 97%, 98%, 99%, or 100%) identity to SEQ ID NO: 1339 or SEQ ID NO: 1341, and pharmaceutically acceptable excipients. Such a tablet may be coated with an enteric coating. Contemplated tablets may include pharmaceutically acceptable excipients such as fillers, binders, disintegrants, and/or lubricants, as well as coloring agents, release agents, coating agents, sweetening, flavoring such as wintergreen, orange, xylitol, sorbitol, fructose, and maltodextrin, and perfuming agents, preservatives and/or antioxidants.

In some embodiments, contemplated pharmaceutical formulations include an intra-granular phase that includes a disclosed STMN2 oligonucleotide, e.g., a STMN2 oligonucleotide represented by any of SEQ ID NOs: 1-466, SEQ ID NO: 893-1338, SEQ ID NOs: 1342-1366, SEQ ID NOs: 1392-1664, and SEQ ID NOs: 10655-10669 that targets a STMN2 transcript comprising a sequence that shares at least 90% (e.g., 90%, 91%, 92%, 93%, 94%, 95%, 96%, 97%, 98%, 99%, or 100%) identity to SEQ ID NO: 1339 or SEQ ID NO: 1341, and a pharmaceutically acceptable salt. In some embodiments, contemplated pharmaceutical formulations include an intra-granular phase that includes a disclosed STMN2 oligonucleotide, e.g., a STMN2 oligonucleotide represented by any of SEQ ID NOs: 1-466, SEQ ID NO: 893-1338, SEQ ID NOs: 1342-1366, SEQ ID NOs: 1392-1664, and SEQ ID NOs: 10655-10669 that targets a STMN2 transcript comprising a sequence that shares at least 90% (e.g., 90%, 91%, 92%, 93%, 94%, 95%, 96%, 97%, 98%, 99%, or 100%) identity to SEQ ID NO: 1339 or SEQ ID NO: 1341, and a pharmaceutically acceptable filler.

For example, a tablet for oral administration is provided that comprises granules (e.g., is at least partially formed from granules) that include a disclosed KCNQ2 oligonucleotide, e.g., a KCNQ2 oligonucleotide represented by any SEQ ID NOs: 1676-1851, SEQ ID NOs: 2028-2529, SEQ ID NOs: 3046-3221, SEQ ID NOs: 3398-3899, and SEQ ID NOs: 4402-4530 that targets a KCNQ2 transcript comprising a sequence that shares at least 90% (e.g., 90%, 91%, 92%, 93%, 94%, 95%, 96%, 97%, 98%, 99%, or 100%) identity to any one of SEQ ID NOs: 3032-3043, and pharmaceutically acceptable excipients. Such a tablet may be coated with an enteric coating. Contemplated tablets may include pharmaceutically acceptable excipients such as fillers, binders, disintegrants, and/or lubricants, as well as coloring agents, release agents, coating agents, sweetening, flavoring such as wintergreen, orange, xylitol, sorbitol, fructose, and maltodextrin, and perfuming agents, preservatives and/or antioxidants.

In some embodiments, contemplated pharmaceutical formulations include an intra-granular phase that includes a disclosed KCNQ2 oligonucleotide, e.g., a KCNQ2 oligonucleotide represented by any of SEQ ID NOs: 1676-1851, SEQ ID NOs: 2028-2529, SEQ ID NOs: 3046-3221, SEQ ID NOs: 3398-3899, and SEQ ID NOs: 4402-4530 that targets a KCNQ2 transcript comprising a sequence that shares at least 90% (e.g., 90%, 91%, 92%, 93%, 94%, 95%, 96%, 97%, 98%, 99%, or 100%) identity to any one of SEQ ID NOs: 3032-3043, and a pharmaceutically acceptable salt. In some embodiments, contemplated pharmaceutical formulations include an intra-granular phase that includes a disclosed KCNQ2 oligonucleotide, e.g., a KCNQ2 oligonucleotide represented by any of SEQ ID NOs: 1676-1851, SEQ ID NOs: 2028-2529, SEQ ID NOs: 3046-3221, SEQ ID NOs: 3398-3899, and SEQ ID NOs: 4402-4530 that targets a KCNQ2 transcript comprising a sequence that shares at least 90% (e.g., 90%, 91%, 92%, 93%, 94%, 95%, 96%, 97%, 98%, 99%, or 100%) identity to any one of SEQ ID NO: 3032-3043, and a pharmaceutically acceptable filler.

For example, a tablet for oral administration is provided that comprises granules (e.g., is at least partially formed from granules) that include a disclosed UNC13A oligonucleotide, e.g., a UNC13A oligonucleotide represented by any SEQ ID NOs: 4531-5794, SEQ ID NO: 7059-8322, SEQ ID NOs: 9596-9696, or SEQ ID NOs: 10670-10779 that targets a UNC13A transcript comprising a sequence that shares at least 90% (e.g., 90%, 91%, 92%, 93%, 94%, 95%, 96%, 97%, 98%, 99%, or 100%) identity to any one of SEQ ID NO: 9587-9595, and pharmaceutically acceptable excipients. Such a tablet may be coated with an enteric coating. Contemplated tablets may include pharmaceutically acceptable excipients such as fillers, binders, disintegrants, and/or lubricants, as well as coloring agents, release agents, coating agents, sweetening, flavoring such as wintergreen, orange, xylitol, sorbitol, fructose, and maltodextrin, and perfuming agents, preservatives and/or antioxidants.

In some embodiments, contemplated pharmaceutical formulations include an intra-granular phase that includes a disclosed UNC13A oligonucleotide, e.g., a UNC13A oligonucleotide represented by any of SEQ ID NOs: 4531-5794, SEQ ID NO: 7059-8322, SEQ ID NOs: 9596-9696, or SEQ ID NOs: 10670-10779 that targets a UNC13A transcript comprising a sequence that shares at least 90% (e.g., 90%, 91%, 92%, 93%, 94%, 95%, 96%, 97%, 98%, 99%, or 100%) identity to any one of SEQ ID NO: 9587-9595, and a pharmaceutically acceptable salt. In some embodiments, contemplated pharmaceutical formulations include an intra-granular phase that includes a disclosed UNC13A oligonucleotide, e.g., a UNC13A oligonucleotide represented by any of SEQ ID NOs: 4531-5794, SEQ ID NO: 7059-8322, SEQ ID NOs: 9596-9696, or SEQ ID NOs: 10670-10779 that targets a UNC13A transcript comprising a sequence that shares at least 90% (e.g., 90%, 91%, 92%, 93%, 94%, 95%, 96%, 97%, 98%, 99%, or 100%) identity to any one of SEQ ID NO: 9587-9595, and a pharmaceutically acceptable filler.

For example, a tablet for oral administration is provided that comprises granules (e.g., is at least partially formed from granules) that include a disclosed SMN2 oligonucleotide, e.g., a SMN2 oligonucleotide represented by any SEQ ID NOs: 9710-10141, SEQ ID NOs: 10574-10651, and SEQ ID NOs: 10783-10808 that targets a SMN2 transcript comprising a sequence that shares at least 90% (e.g., 90%, 91%, 92%, 93%, 94%, 95%, 96%, 97%, 98%, 99%, or 100%) identity to any one of SEQ ID NOs: 9698-9709, and pharmaceutically acceptable excipients. Such a tablet may be coated with an enteric coating. Contemplated tablets may include pharmaceutically acceptable excipients such as fillers, binders, disintegrants, and/or lubricants, as well as coloring agents, release agents, coating agents, sweetening, flavoring such as wintergreen, orange, xylitol, sorbitol, fructose, and maltodextrin, and perfuming agents, preservatives and/or antioxidants.

In some embodiments, contemplated pharmaceutical formulations include an intra-granular phase that includes a disclosed SMN2 oligonucleotide, e.g., a SMN2 oligonucleotide represented by any of SEQ ID NOs: 9710-10141, SEQ ID NOs: 10574-10651, and SEQ ID NOs: 10783-10808 that targets a SMN2 transcript comprising a sequence that shares at least 90% (e.g., 90%, 91%, 92%, 93%, 94%, 95%, 96%, 97%, 98%, 99%, or 100%) identity to any one of SEQ ID NOs: 9698-9707, and a pharmaceutically acceptable salt. In some embodiments, contemplated pharmaceutical formulations include an intra-granular phase that includes a disclosed SMN2 oligonucleotide, e.g., a SMN2 oligonucleotide represented by any of SEQ ID NOs: 9710-10141, SEQ ID NOs: 10574-10651, and SEQ ID NOs: 10783-10808 that targets a SMN2 transcript comprising a sequence that shares at least 90% (e.g., 90%, 91%, 92%, 93%, 94%, 95%, 96%, 97%, 98%, 99%, or 100%) identity to any one of SEQ ID NO: 9698-9709, and a pharmaceutically acceptable filler.

For example, a disclosed STMN2 oligonucleotide, KCNQ2 oligonucleotide, UNC13A oligonucleotide, and/or SMN2 oligonucleotide and a filler may be blended together, optionally, with other excipients, and formed into granules. In some embodiments, the intragranular phase may be formed using wet granulation, e.g., a liquid (e.g., water) is added to the blended STMN2 oligonucleotide, KCNQ2 oligonucleotide, UNC13A oligonucleotide, and/or SMN2 oligonucleotide and filler, and then the combination is dried, milled and/or sieved to produce granules. One of skill in the art would understand that other processes may be used to achieve an intragranular phase.

In some embodiments, contemplated formulations include an extra-granular phase, which may include one or more pharmaceutically acceptable excipients, and which may be blended with the intragranular phase to form a disclosed formulation.

A disclosed formulation may include an intragranular phase that includes a filler. Exemplary fillers include, but are not limited to, cellulose, gelatin, calcium phosphate, lactose, sucrose, glucose, mannitol, sorbitol, microcrystalline cellulose, pectin, polyacrylates, dextrose, cellulose acetate, hydroxypropylmethyl cellulose, partially pre-gelatinized starch, calcium carbonate, and others including combinations thereof.

In some embodiments, a disclosed formulation may include an intragranular phase and/or an extragranular phase that includes a binder, which may generally function to hold the ingredients of the pharmaceutical formulation together. Exemplary binders of the disclosure may include, but are not limited to, the following: starches, sugars, cellulose or modified cellulose such as hydroxypropyl cellulose, lactose, pre-gelatinized maize starch, polyvinyl pyrrolidone, hydroxypropyl cellulose, hydroxypropylmethyl cellulose, low substituted hydroxypropyl cellulose, sodium carboxymethyl cellulose, methyl cellulose, ethyl cellulose, sugar alcohols and others including combinations thereof.

Contemplated formulations, e.g., that include an intragranular phase and/or an extragranular phase, may include a disintegrant such as but not limited to, starch, cellulose, crosslinked polyvinyl pyrrolidone, sodium starch glycolate, sodium carboxymethyl cellulose, alginates, corn starch, croscarmellose sodium, crosslinked carboxymethyl cellulose, low substituted hydroxypropyl cellulose, acacia, and others including combinations thereof. For example, an intragranular phase and/or an extragranular phase may include a disintegrant.

In some embodiments, a contemplated formulation includes an intra-granular phase comprising a disclosed STMN2 oligonucleotide, KCNQ2 oligonucleotide, UNC13A oligonucleotide, and/or SMN2 oligonucleotide and excipients chosen from: mannitol, microcrystalline cellulose, hydroxypropylmethyl cellulose, and sodium starch glycolate or combinations thereof, and an extra-granular phase comprising one or more of: microcrystalline cellulose, sodium starch glycolate, and magnesium stearate or mixtures thereof.

In some embodiments, a contemplated formulation may include a lubricant, e.g. an extra-granular phase may contain a lubricant. Lubricants include but are not limited to talc, silica, fats, stearin, magnesium stearate, calcium phosphate, silicone dioxide, calcium silicate, calcium phosphate, colloidal silicon dioxide, metallic stearates, hydrogenated vegetable oil, corn starch, sodium benzoate, polyethylene glycols, sodium acetate, calcium stearate, sodium lauryl sulfate, sodium chloride, magnesium lauryl sulfate, talc, and stearic acid.

In some embodiments, the pharmaceutical formulation comprises an enteric coating. Generally, enteric coatings create a barrier for the oral medication that controls the location at which the drug is absorbed along the digestive track. Enteric coatings may include a polymer that disintegrates at different rates according to pH. Enteric coatings may include for example, cellulose acetate phthalate, methyl acrylate-methacrylic acid copolymers, cellulose acetate succinate, hydroxypropylmethyl cellulose phthalate, methyl methacrylate-methacrylic acid copolymers, ethylacrylate-methacrylic acid copolymers, methacrylic acid copolymer type C, polyvinyl acetate-phthalate, and cellulose acetate phthalate.

Exemplary enteric coatings include Opadry® AMB, Acryl-EZE®, Eudragit® grades. In some embodiments, an enteric coating may comprise about 5% to about 10%, about 5% to about 20%, 8% to about 15%, about 8% to about 20%, about 10% to about 20%, or about 12% to about 20%, or about 18% of a contemplated tablet by weight. For example, enteric coatings may include an ethylacrylate-methacrylic acid copolymer.

For example, in a contemplated embodiment, a tablet is provided that comprises or consists essentially of about 0.5% to about 70%, e.g., about 0.5% to about 10%, or about 1% to about 20%, by weight of a disclosed STMN2 oligonucleotide, KCNQ2 oligonucleotide, UNC13A oligonucleotide, and/or SMN2 oligonucleotide or a pharmaceutically acceptable salt thereof. Such a tablet may include for example, about 0.5% to about 60% by weight of mannitol, e.g., about 30% to about 50% by weight mannitol, e.g., about 40% by weight mannitol; and/or about 20% to about 40% by weight of microcrystalline cellulose, or about 10% to about 30% by weight of microcrystalline cellulose. For example, a disclosed tablet may comprise an intragranular phase that includes about 30% to about 60%, e.g. about 45% to about 65% by weight, or alternatively, about 5 to about 10% by weight of a disclosed STMN2 oligonucleotide, KCNQ2 oligonucleotide, UNC13A oligonucleotide, and/or SMN2 oligonucleotide, about 30% to about 50%, or alternatively, about 5% to about 15% by weight mannitol, about 5% to about 15% microcrystalline cellulose, about 0% to about 4%, or about 1% to about 7% hydroxypropylmethylcellulose, and about 0% to about 4%, e.g., about 2% to about 4% sodium starch glycolate by weight.

In another contemplated embodiment, a pharmaceutical tablet formulation for oral administration of a disclosed STMN2 oligonucleotide, KCNQ2 oligonucleotide, UNC13A oligonucleotide, and/or SMN2 oligonucleotide comprises an intra-granular phase, wherein the intra-granular phase includes a disclosed STMN2 AON, KCNQ2 AON, UNC13A AON, and/or SMN2 AON or a pharmaceutically acceptable salt thereof (such as a sodium salt), and a pharmaceutically acceptable filler, and which may also include an extra-granular phase, that may include a pharmaceutically acceptable excipient such as a disintegrant. The extra-granular phase may include components chosen from microcrystalline cellulose, magnesium stearate, and mixtures thereof. The pharmaceutical composition may also include an enteric coating of about 12% to 20% by weight of the tablet. For example, a pharmaceutically acceptable tablet for oral use may comprise about 0.5% to 10% by weight of a disclosed STMN2 AON, KCNQ2 AON, UNC13A AON, and/or SMN2 AON, e.g., a disclosed STMN2 AON, KCNQ2 AON, UNC13A AON, and/or SMN2 AON or a pharmaceutically acceptable salt thereof, about 30% to 50% by weight mannitol, about 10% to 30% by weight microcrystalline cellulose, and an enteric coating comprising an ethylacrylate-methacrylic acid copolymer.

In another example, a pharmaceutically acceptable tablet for oral use may comprise an intra-granular phase, comprising about 5 to about 10% by weight of a disclosed STMN2 AON, e.g., a disclosed STMN2 AON or a pharmaceutically acceptable salt thereof, about 40% by weight mannitol, about 8% by weight microcrystalline cellulose, about 5% by weight hydroxypropylmethyl cellulose, and about 2% by weight sodium starch glycolate; an extra-granular phase comprising about 17% by weight microcrystalline cellulose, about 2% by weight sodium starch glycolate, about 0.4% by weight magnesium stearate; and an enteric coating over the tablet comprising an ethylacrylate-methacrylic acid copolymer.

In some embodiments the pharmaceutical composition may contain an enteric coating comprising about 13% or about 15%, 16%, 17% or 18% by weight, e.g., AcyrlEZE® (see. e.g., PCT Publication No. WO 2010/054826, which is hereby incorporated by reference in its entirety).

The rate at which the coating dissolves and the active ingredient is released is its dissolution rate. In an embodiment, a contemplated tablet may have a dissolution profile, e.g., when tested in a USP/EP Type 2 apparatus (paddle) at 100 rpm and 37° C. in a phosphate buffer with a pH of 7.2, of about 50% to about 100% of the STMN2 oligonucleotide releasing after about 120 minutes to about 240 minutes, for example after 180 minutes. In another embodiment, a contemplated tablet may have a dissolution profile, e.g., when tested in a USP/EP Type 2 apparatus (paddle) at 100 rpm and 37° C. in diluted HCl with a pH of 1.0, where substantially none of the STMN2 oligonucleotide is released after 120 minutes. A contemplated tablet, in another embodiment, may have a dissolution profile, e.g., when tested in USP/EP Type 2 apparatus (paddle) at 100 rpm and 37° C. in a phosphate buffer with a pH of 6.6, of about 10% to about 30%, or not more than about 50% of the STMN2 oligonucleotide, KCNQ2 oligonucleotide, UNC13A oligonucleotide, and/or SMN2 oligonucleotide releasing after 30 minutes.

In some embodiments, methods provided herein may further include administering at least one other agent that is directed to treatment of diseases and disorders disclosed herein. In one embodiment, contemplated other agents may be co-administered (e.g., sequentially or simultaneously).

### Dosage and Frequency of Administration

The dosage or amounts described below refer either to the oligonucleotide or a pharmaceutically acceptable salt thereof.

In some embodiments, methods described herein include administering at least 1 μg, at least 5 μg, at least 10 μg, at least 20 μg, at least 30 μg, at least 40 μg, at least 50 μg, at least 60 μg, at least 70 μg, at least 80 μg, at least 90 μg, or at least 100 μg of a STMN2 antisense oligonucleotide, KCNQ2 antisense oligonucleotide, UNC13A antisense oligonucleotide, and/or SMN2 antisense oligonucleotide e.g., a STMN2 oligonucleotide, KCNQ2 oligonucleotide, UNC13A oligonucleotide, and/or SMN2 oligonucleotide. In some embodiments, methods include administering from 10 mg to 500 mg, from 1 mg to 10 mg, from 10 mg to 20 mg, from 20 mg to 30 mg, from 30 mg to 40 mg, from 40 mg to 50 mg, from 50 mg to 60 mg, from 60 mg to 70 mg, from 70 mg to 80 mg, from 80 mg to 90 mg, from 90 mg to 100 mg, from 100 mg to 150 mg, from 150 mg to 200 mg, from 200 mg to 250 mg, from 250 mg to 300 mg, from 300 mg to 350 mg, from 350 mg to 400 mg, from 400 mg to 450 mg, from 450 mg to 500 mg, from 500 mg to 600 mg, from 600 mg to 700 mg, from 700 mg to 800 mg, from 800 mg to 900 mg, from 900 mg to 1 g, from 1 mg to 50 mg, from 20 mg to 40 mg, or from 1 mg to 500 mg of a STMN2 antisense oligonucleotide.

In some embodiments, methods described herein include administering formulations that include about 10 mg, 15 mg, 20 mg, 25 mg, 30 mg, 35 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 250 mg, 300 mg, 350 mg, 400 mg, 450 mg, 500 mg, 600 mg, 700 mg, 800 mg, 900 mg, 1 g, 1.5 g, 2.0 g, 2.5 g, 3.0 g, 3.5 g, 4.0 g, 4.5 g, or 5.0 g of a disclosed STMN2 oligonucleotide. In some embodiments, a formulation may include about 40 mg, 80 mg, or 160 mg of a disclosed STMN2 oligonucleotide, KCNQ2 oligonucleotide, UNC13A oligonucleotide, and/or SMN2 oligonucleotide e. In some embodiments, a formulation may include at least 100 μg of a disclosed STMN2 oligonucleotide. For example, formulations may include about 0.1 mg, 0.2 mg, 0.3 mg, 0.4 mg, 0.5 mg, 1 mg, 5 mg, 10 mg, 15 mg, 20 mg, 25 mg, or 30 mg of a disclosed STMN2 oligonucleotide, KCNQ2 oligonucleotide, UNC13A oligonucleotide, and/or SMN2 oligonucleotide. The amount administered will depend on variables such as the type and extent of disease or indication to be treated, the overall health and size of the patient, the in vivo potency of the STMN2 oligonucleotide, the pharmaceutical formulation, and the route of administration. The initial dosage can be increased beyond the upper level in order to rapidly achieve the desired blood-level or tissue level. Alternatively, the initial dosage can be smaller than the optimum, and the dosage may be progressively increased during the course of treatment. Human dosage can be optimized, e.g., in a conventional Phase I dose escalation study. Dosing frequency can vary, depending on factors such as route of administration, dosage amount and the disease being treated. Exemplary dosing frequencies are once per day, once per week and once every two weeks. In some embodiments, dosing is once per day for 7 days. In some embodiments, dosing is once every 4 weeks, once every 5 weeks, once every 6 weeks, once every 7 weeks, once every 8 weeks, once every 9 weeks, once every 10 weeks, once every 11 weeks, or once every 12 weeks. In some embodiments, dosing is once a month to every three months.

### Combination Therapies

In various embodiments, a STMN2 AON, KCNQ2 AON, UNC13A AON, and/or SMN2 AON as disclosed herein can be administered in combination with one or more additional therapies. The combination therapy of the disclosed oligonucleotide and the one or more additional therapies can, in some embodiments, be synergistic in treating any of amyotrophic lateral sclerosis (ALS), frontotemporal dementia (FTD), ALS with FTD, Alzheimer's disease (AD), Parkinson's disease (PD), Huntington's disease, progressive supranuclear palsy (PSP), brain trauma, spinal cord injury, corticobasal degeneration (CBD), nerve injuries (e.g., brachial plexus injuries), neuropathies (e.g., chemotherapy induced neuropathy), and TDP43 proteinopathies (e.g., chronic traumatic encephalopathy, Perry Syndrome, Dementia with Lewy body in association with Parkinson's disease, Parkinson's disease with or without dementia, Limbic-predominant age-related TDP-43 encephalopathy (LATE)) Cerebral Age-Related TDP-43 With Sclerosis (CARTS), facial onset sensory and motor neuronopathy, Guam Parkinson-dementia complex, multisystem proteinopathy, CTE, amyotrophic lateral sclerosis (ALS or Lou Gehrig's disease), progressive bulbar palsy, pseudobulbar palsy, progressive muscular atrophy, primary lateral sclerosis, spinal muscular atrophy, post-polio syndrome, and synaptic diseases like autism.

Non-limiting examples of therapies for Parkinson's disease (PD) include: deep brain stimulation, levodopa and carbidopa (duopa, rytary, Sinemet, inbrija), istradefylline (nourianz), safinamide (xadago), pramipexole (Mirapex), rotigotine (neupro), ropinirole (requip), amantadine (gocovri, Symmetrel, osmolex), benztropine (Cogentin), trihexyphenidyl (artane), selegiline (eldepryl, zelapar), rasagiline, entacapone (comtan), opicapone (ongentys), tolcapone (tasmar), apomorphine (apokyn, kynmobi), exenatide, lingzhi, BIIB054, BIIB094, Caffeine, sarizotan, Nuplazid and embryonic dopamine cell implantation.

Non-limiting examples of therapies for Alzheimer's disease (AD) include aducanamab (Aduhlem), memantine (Namenda), Donepezil (Aricept), Rivastigmine (Exelon), Galantamine (razadyne), Namzeric, Suvorexant (belsomra), and lecanemab.

A non-limiting example of a therapy for amyotrophic lateral sclerosis (ALS) is pridopidine.

Non-limiting examples of therapies for frontotemporal dementia (FTD) include olanzapine (Zyprexa), quetiapine (Seroquel), SSRIs (citalopram (Cipramil), dapoxetine (Priligy), escitalopram (Cipralex), fluoxetine (Prozac or Oxactin), fluvoxamine (Faverin), paroxetine (Seroxat), sertraline (Lustral), vortioxetine (Brintellix)), divalproex sodium (Depakote), carbamazepine (Tegretol), and medroxyprogestrone.

Example additional therapies include any of Riluzole (Rilutek), PrimeC, Edaravone (Radicava), rivastigmine, donepezil, galantamine, selective serotonin reuptake inhibitor, antipsychotic agents, cholinesterase inhibitors, memantine, benzodiazepine antianxiety drugs, AMX0035 (ELYBRIO), ZILUCOPLAN (RA101495), pridopidine, dual AON intrathecal administration (e.g., BIIB067, BIIB078, and BIIB105), BIIB100, levodopa/carbidopa, dopaminergic agents (e.g., ropinirole, pramipexole, rotigotine), medroxyprosterone, KCNQ2/KCNQ3 openers (e.g., retigabine, XEN1101, or QRL-101), bioactive scaffolds, anticonvulsants and psychostimulant agents. Additional therapies can further include breathing care, physical therapy, occupational therapy, speech therapy, and nutritional support. Further non-limiting examples of additional therapies include any of deep brain stimulation, levodopa and carbidopa (duopa, rytary, Sinemet, inbrija), istradefylline (nourianz), safinamide (xadago), pramipexole (Mirapex), rotigotine (neupro), ropinirole (requip), amantadine (gocovri, Symmetrel, osmolex), benztropine (Cogentin), trihexyphenidyl (artane), selegiline (eldepryl, zelapar), rasagiline, entacapone (comtan), opicapone (ongentys), tolcapone (tasmar), apomorphine (apokyn, kynmobi), exenatide, lingzhi, BIIB054, BIIB094, Caffeine, sarizotan, embryonic dopamine cell implantation, aducanamab (Aduhlem), memantine (Namenda), Donepezil (Aricept), Rivastigmine (Exelon), Galantamine (razadyne), Namzeric, Suvorexant (belsomra), lecanemab, olanzapine (Zyprexa), quetiapine (Seroquel), SSRIs (citalopram (Cipramil), dapoxetine (Priligy), escitalopram (Cipralex), fluoxetine (Prozac or Oxactin), fluvoxamine (Faverin), paroxetine (Seroxat), sertraline (Lustral), vortioxetine (Brintellix))), divalproex sodium (Depakote), carbamazepine (Tegretol), medroxyprogestrone, Brivaracetam (briviact), cannabidiol (epidiolex), carbamazepine (carbatrol, Tegretol), cenobamate (xcopri), diazepam (valium), lorazepam (Ativan), clonazepam (klonopin), eslicarbazepine (aptiom), ethosuximide (zarontin), felbamate (felbatol), fenfluramine (fintepla), lacosamide (VIMPAT), lamotrigine (Lamictal), levetiracetam (Keppra), oxcarbazepine (oxtellar xr, Trileptal), perampanel (fycompa), phenobarbital, phenytoin (dilantin), pregabalin (lyrica), tiagabine (gabitril), topiramate (topamax), valproate (depakene, depakote), and zonisamide (zonegran). In various embodiments, an additional therapy can be a second antisense oligonucleotide. As an example, the second antisense oligonucleotide may target a STMN2 transcript, KCNQ2 transcript, UNC13A transcript, and/or SMN2 transcript (e.g., STMN2 pre-mRNA, KCNQ2 pre-mRNA, UNC13A pre-mRNA, and/or SMN2 pre-mRNA, mature STMN2 mRNA, KCNQ2 mRNA, UNC13A mRNA, and/or SMN2 mRNA) to modulate the expression levels of full length STMN2 protein, KCNQ2 protein, UNC13A protein, and/or SMN2 protein.

Non-limiting examples of therapies for spinal cord injury includes bioactive scaffolds, such as bioactive scaffolds with enhanced supramolecular motion. Further details of example bioactive scaffolds as therapies for spinal cord injury is described in Alvarez et al., “Bioactive scaffolds with enhanced supramolecular motion promote recovery from spinal cord injury.” Science, 374, 848-856 (2021), which is hereby incorporated by reference in its entirety.

Example additional therapies for spinal muscular atrophy include any of nusinersen (spinraza), onasemnogene abeparvovec-xioi (zolgensma), and risdiplam (evrysdi).

In various embodiments, the disclosed oligonucleotide and the one or more additional therapies can be conjugated to one another and provided in a conjugated form. Further description regarding conjugates involving the disclosed oligonucleotide is described below. In various embodiments, the disclosed oligonucleotide and one or more additional therapies are provided concurrently. In various embodiments, the disclosed oligonucleotide and one or more additional therapies are provided simultaneously. In various embodiments, the disclosed oligonucleotide and one or more additional therapies are provided sequentially.

### Conjugates

In certain embodiments, provided herein are oligomeric compounds, which comprise an oligonucleotide (e.g., STMN2 oligonucleotide, KCNQ2 oligonucleotide, UNC13A oligonucleotide, and/or SMN2 oligonucleotide) and optionally one or more conjugate groups and/or terminal groups. Conjugate groups include one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, modified or unmodified nucleosides, and two or more nucleosides that are independently modified or unmodified.

### Conjugate Groups

In certain embodiments, a STMN2 AON, KCNQ2 AON, UNC13A AON, and/or SMN2 AON is covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge, and clearance. In particular embodiments, conjugate groups modify the circulation time (e.g., increase) of the oligonucleotides in the bloodstream such that increased concentrations of the oligonucleotides are delivered to the brain. In particular embodiments, conjugate groups modify the residence time (e.g., increase residence time) of the oligonucleotides in a target organ (e.g., brain) such that increased residence time of the oligonucleotides improves their performance (e.g., efficacy). In particular embodiments, conjugate groups increase the delivery of the oligonucleotide to the brain through the blood brain barrier and/or brain parenchyma (e.g., through receptor mediated transcytosis). In particular embodiments, conjugate groups enable the oligonucleotide to target a specific organ (e.g., the brain). In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. NY. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

### Conjugate Moieties

Conjugate moieties include, without limitation, intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, dyes, bile acids, and phenylbutyric acid. In particular embodiments, conjugate moieties are selected from a peptide, a lipid, N-acetylgalactosamine (GalNAc), cholesterol, vitamin E, lipoic acid, panthothenic acid, polyethylene glycol, an antibody (e.g., an antibody for crossing the blood brain barrier such as anti-transferrin receptor antibody), or a cell-penetrating peptide (e.g., transactivator of transcription (TAT) and penetratine).

In certain embodiments, a conjugate moiety comprises an active drug substance, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indomethacin, a barbiturate, a cephalosporin, a sulfa drug, an antidiabetic, an antibacterial or an antibiotic.

### Conjugate Linkers

Conjugate moieties are attached to a STMN2 AON, KCNQ2 AON, UNC13A AON, and/or SMN2 AON through conjugate linkers. In certain oligomeric compounds, the conjugate linker is a single chemical bond (i.e., the conjugate moiety is attached directly to an oligonucleotide through a single bond). In certain embodiments, the conjugate linker comprises a chain structure, such as a hydrocarbon chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise 3 linker-nucleosides.

In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methyl cytosine, 4-N-benzoyl-5-methyl cytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid.

In certain embodiments, it is desirable for a conjugate group to be cleaved from the AON. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxy adenosine.

### Terminal Groups

In certain embodiments, oligomeric compounds comprise one or more terminal groups. In certain such embodiments, oligomeric compounds comprise a stabilized 5′-phosphate. Stabilized 5′-phosphates include, but are not limited to 5′-phosphonates, including, but not limited to 5′-vinylphosphonates. In certain embodiments, terminal groups comprise one or more abasic nucleosides and/or inverted nucleosides. In certain embodiments, terminal groups comprise one or more 2′-linked nucleosides. In certain such embodiments, the 2′-linked nucleoside is an abasic nucleoside. In various embodiments, terminal groups comprise one or more spacers.

### Diagnostic Methods—STMN2

The disclosure also provides a method of diagnosing a patient with a neurological disease that relies upon detecting levels of STMN2 expression signal in one or more biological samples of a patient. As used herein, the term “STMN2 expression signal” can refer to any indication of STMN2 gene expression, or gene or gene product activity. STMN2 gene products include RNA (e.g., mRNA), peptides, and proteins. Indices of STMN2 gene expression that can be assessed include, but are not limited to, STMN2 gene or chromatin state, STMN2 gene interaction with cellular components that regulate gene expression, STMN2 gene product expression levels (e.g., expression levels of STMN2 transcripts (for example, a STMN2 pre-mRNA comprising a cryptic exon) comprising a sequence that shares at least 90% (e.g., 90%, 91%, 92%, 93%, 94%, 95%, 96%, 97%, 98%, 99%, or 100%) identity to SEQ ID NO: 1339 or SEQ ID NO: 1341, or interaction of STMN2 RNA or protein with transcriptional, translational, or post-translational processing machinery.

Detection of STMN2 expression signal may be accomplished through in vivo, in vitro, or ex vivo methods. In a preferred embodiment, methods of the disclosure may be carried out in vitro. Methods of detecting may involve detection in blood, serum, fecal matter, tissue, cerebrospinal fluid, spinal fluid, extracellular vesicles (for example, CSF exosomes), or cells of a patient. Detection may be achieved by measuring expression signal of STMN2 transcripts (for example, a STMN2 pre-mRNA comprising a cryptic exon) comprising a sequence that shares at least 90% (e.g., 90%, 91%, 92%, 93%, 94%, 95%, 96%, 97%, 98%, 99%, or 100%) identity to SEQ ID NO: 1339 or SEQ ID NO: 1341 in whole tissue, tissue explants, cell cultures, dissociated cells, cell extract, extracellular vesicles (for example, CSF exosomes), or body fluids, including blood, spinal fluid, cerebrospinal fluid, urine, lymphatic fluid, or serum. Methods of detection include assays that measure levels of STMN2 gene product expression such as Western blotting, FACS, ELISA, other quantitative binding assays, cell or tissue growth assays, Northern blots, quantitative or semi-quantitative polymerase chain reaction, medical imaging methods (e.g., MRI), or immunostaining methods (e.g., immunohistochemistry or immunocytochemistry).

### Diagnostic Methods—KCNQ2

The disclosure also provides a method of diagnosing a patient with a neurological disease that relies upon detecting levels of KCNQ2 expression signal in one or more biological samples of a patient. As used herein, the term “KCNQ2 expression signal” can refer to any indication of KCNQ2 gene expression, or gene or gene product activity. KCNQ2 gene products include RNA (e.g., mRNA), peptides, and proteins. Indices of KCNQ2 gene expression that can be assessed include, but are not limited to, KCNQ2 gene or chromatin state, KCNQ2 gene interaction with cellular components that regulate gene expression, KCNQ2 gene product expression levels (e.g., expression levels of KCNQ2 transcripts (for example, a KCNQ2 pre-mRNA) comprising a sequence that shares at least 90% (e.g., 90%, 91%, 92%, 93%, 94%, 95%, 96%, 97%, 98%, 99%, or 100%) identity to any one of SEQ ID NOs: 3032-3045, or interaction of KCNQ2 RNA or protein with transcriptional, translational, or post-translational processing machinery.

Detection of KCNQ2 expression signal may be accomplished through in vivo, in vitro, or ex vivo methods. In a preferred embodiment, methods of the disclosure may be carried out in vitro. Methods of detecting may involve detection in blood, serum, fecal matter, tissue, cerebrospinal fluid, spinal fluid, urine, extracellular vesicles (for example, CSF exosomes), or cells of a patient. Detection may be achieved by measuring expression signal of KCNQ2 transcripts (for example, a KCNQ2 pre-mRNA) comprising a sequence that shares at least 90% (e.g., 90%, 91%, 92%, 93%, 94%, 95%, 96%, 97%, 98%, 99%, or 100%) identity to any one of SEQ ID NOs: 3032-3045 in whole tissue, tissue explants, cell cultures, dissociated cells, cell extract, extracellular vesicles (for example, CSF exosomes), or body fluids, including blood, spinal fluid, cerebrospinal fluid, urine, lymphatic fluid, or serum. Methods of detection include assays that measure levels of KCNQ2 gene product expression such as Western blotting, FACS, ELISA, other quantitative binding assays, cell or tissue growth assays, Northern blots, quantitative or semi-quantitative polymerase chain reaction, medical imaging methods (e.g., MRI), or immunostaining methods (e.g., immunohistochemistry or immunocytochemistry).

### Diagnostic Methods—UNC13A

The disclosure also provides a method of diagnosing a patient with a neurological disease that relies upon detecting levels of UNC13A expression signal in one or more biological samples of a patient. As used herein, the term “UNC13A expression signal” can refer to any indication of UNC13A gene expression, or gene or gene product activity. UNC13A gene products include RNA (e.g., mRNA), peptides, and proteins. Indices of UNC13A gene expression that can be assessed include, but are not limited to, UNC13A gene or chromatin state, UNC13A gene interaction with cellular components that regulate gene expression, UNC13A gene product expression levels (e.g., expression levels of UNC13A transcripts (for example, a UNC13A pre-mRNA) comprising a sequence that shares at least 90% (e.g., 90%, 91%, 92%, 93%, 94%, 95%, 96%, 97%, 98%, 99%, or 100%) identity to any one of SEQ ID NO: 9587-9595, or interaction of UNC13A RNA or protein with transcriptional, translational, or post-translational processing machinery.

Detection of UNC13A expression signal may be accomplished through in vivo, in vitro, or ex vivo methods. In a preferred embodiment, methods of the disclosure may be carried out in vitro. Methods of detecting may involve detection in blood, serum, fecal matter, tissue, cerebrospinal fluid, spinal fluid, urine, extracellular vesicles (for example, CSF exosomes), or cells of a patient. Detection may be achieved by measuring expression signal of UNC13A transcripts (for example, a UNC13A pre-mRNA) comprising a sequence that shares at least 90% (e.g., 90%, 91%, 92%, 93%, 94%, 95%, 96%, 97%, 98%, 99%, or 100%) identity to SEQ ID NO: 9587-9595 in whole tissue, tissue explants, cell cultures, dissociated cells, cell extract, extracellular vesicles (for example, CSF exosomes), or body fluids, including blood, spinal fluid, cerebrospinal fluid, urine, lymphatic fluid, plasma, or serum. Methods of detection include assays that measure levels of UNC13A gene product expression such as Western blotting, FACS, ELISA, other quantitative binding assays, cell or tissue growth assays, Northern blots, quantitative or semi-quantitative polymerase chain reaction, medical imaging methods (e.g., MRI), or immunostaining methods (e.g., immunohistochemistry or immunocytochemistry).

### Diagnostic Methods—SMN2

The disclosure also provides a method of diagnosing a patient with a neurological disease that relies upon detecting levels of SMN2 expression signal in one or more biological samples of a patient. As used herein, the term “SMN2 expression signal” can refer to any indication of SMN2 gene expression, or gene or gene product activity. SMN2 gene products include RNA (e.g., mRNA), peptides, and proteins. Indices of SMN2 gene expression that can be assessed include, but are not limited to, SMN2 gene or chromatin state, SMN2 gene interaction with cellular components that regulate gene expression, SMN2 gene product expression levels (e.g., expression levels of SMN2 transcripts (for example, a SMN2 pre-mRNA) comprising a sequence that shares at least 90% (e.g., 90%, 91%, 92%, 93%, 94%, 95%, 96%, 97%, 98%, 99%, or 100%) identity to any one of SEQ ID NOs: 9698-9709, or interaction of SMN2 RNA or protein with transcriptional, translational, or post-translational processing machinery.

Detection of SMN2 expression signal may be accomplished through in vivo, in vitro, or ex vivo methods. In a preferred embodiment, methods of the disclosure may be carried out in vitro. Methods of detecting may involve detection in blood, serum, fecal matter, tissue, cerebrospinal fluid, spinal fluid, urine, extracellular vesicles (for example, CSF exosomes), or cells of a patient. Detection may be achieved by measuring expression signal of SMN2 transcripts (for example, a SMN2 pre-mRNA) comprising a sequence that shares at least 90% (e.g., 90%, 91%, 92%, 93%, 94%, 95%, 96%, 97%, 98%, 99%, or 100%) identity to any one of SEQ ID NOs: 9698-9707 in whole tissue, tissue explants, cell cultures, dissociated cells, cell extract, extracellular vesicles (for example, CSF exosomes), or body fluids, including blood, spinal fluid, cerebrospinal fluid, urine, lymphatic fluid, or serum. Methods of detection include assays that measure levels of SMN2 gene product expression such as Western blotting, FACS, ELISA, other quantitative binding assays, cell or tissue growth assays, Northern blots, quantitative or semi-quantitative polymerase chain reaction, medical imaging methods (e.g., MRI), or immunostaining methods (e.g., immunohistochemistry or immunocytochemistry).

### Modifications in General

While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (methylated uracil) in place of a uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG,” wherein ^{m}C indicates a cytosine base comprising a methyl group at the 5-position.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as α or β such as for sugar anomers, or as (D) or (L), such as for amino acids, etc. Compounds provided herein that are drawn or described as having certain stereoisomeric configurations include only the indicated compounds. Compounds provided herein that contain stereocenters that are drawn or described with undefined stereochemistry included all such possible isomers, including their stereorandom and optically pure forms, unless specified otherwise. Likewise, all tautomeric forms of the compounds herein are also included unless otherwise indicated. Unless otherwise indicated, compounds described herein are intended to include corresponding salt forms.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool.

### EXAMPLES

The disclosure is further illustrated by the following examples. The examples are provided for illustrative purposes only, and are not to be construed as limiting the scope or content of the disclosure in any way.

### Example 1: Design and Selection of STMN2 Oligonucleotides

STMN2 AONs oligonucleotides that target a STMN2 transcript including a cryptic exon are designed and tested to identify STMN2 AONs capable of reducing quantity of STMN2 transcripts that comprise a cryptic exon. Such STMN2 AONs include STMN2 parent oligonucleotides represented by any of SEQ ID NOs: 1-446 or SEQ ID NOs: 893-1338. The STMN2 parent oligonucleotides are 25 nucleosides in length. Each of the nucleosides of the STMN2 parent oligonucleotides are modified nucleosides with 2′-MOE sugar moieties, and each “C” is replaced with a 5-MeC. Additionally, each of the internucleoside linkages between the nucleosides of the STMN2 oligonucleotides are phosphorothioate internucleoside linkages.

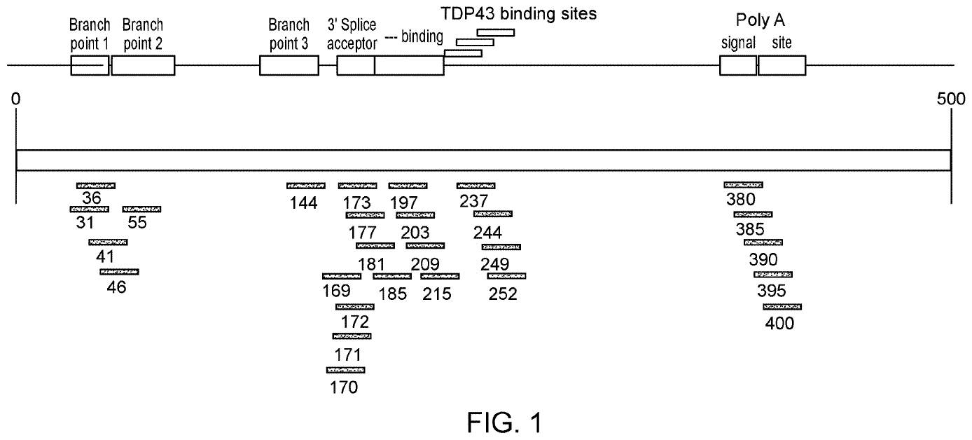

FIG. 1 is a depiction of portions of the STMN2 transcript and STMN2 parent oligonucleotides that are designed to target certain portions of the STMN2 transcript including a cryptic exon. Specifically, regions of the STMN2 transcript include branch points (e.g., branch point 1, 2, and 3) a 3′ splice acceptor region, an ESE binding region, TDP43 binding sites, and a Poly A region. STMN2 oligonucleotides, are identified according to the position of the STMN2 transcript that the STMN2 oligonucleotide corresponds to. For example, FIG. 1 depicts a STMN2 oligonucleotide that targets positions 36 to 60 of the STMN2 transcript including a cryptic exon, which includes a branch point 1. Similarly, a different STMN2 oligonucleotide targets positions 144 to 178 of the STMN2 transcript including a cryptic exon, which includes a branch point 3. Other STMN2 oligonucleotides can be designed using any of the sequences disclosed above.

Generally, the length of the STMN2 antisense oligonucleotides are 25 nucleotide bases in length. However, variants of the STMN2 antisense oligonucleotides were also designed with varying lengths (e.g., 23mers, 21mers, or 19mers). Examples of these variant STMN2 antisense oligonucleotides were designed to include the sequences of SEQ ID NOs: 1342-1366 or SEQ ID NOs: 1392-1521.

### Example 2: Methods for Evaluating STMN2 Antisense Oligonucleotides

STMN2 antisense oligonucleotides were evaluated in SY5Y cells and human motor neurons (hMN). The cells were plated in 6-well or 96-well plates and cultured to 80% confluency. Antisense oligonucleotide (AON) to TDP43 was transfected with RNAiMax (Thermo Fisher Scientific, Waltham, MA, USA) to express the cryptic exon, thus preventing transcription of full-length STMN2 (STMN2-FL) product. Vehicle was treated with RNAiMax alone. Positive controls included cells that were treated with TDP43 siRNA alone (“siRNA TDP43”) and/or TDP43 AON alone (“AON TDP43” or “TDP43 AON”). siRNA TDP43 was purchased as ON-TARGETplus Human TARDBP (23435) siRNA—SMARTpool (#L-012394-00-0005) from Horizon/Dharmacon. TARDBP (23435) siRNA includes four individual siRNAs that targets four separate sequences:

1) Target sequence 1:
(SEQ ID NO: 1665)
GCUCAAGCAUGGAUUCUAA
2) Target sequence 2:
(SEQ ID NO: 1666)
CAAUCAAGGUAGUAAUAUG
3) Target sequence 3:
(SEQ ID NO: 1667)
GGGCUUCGCUACAGGAAUC
4) Target sequence 4:
(SEQ ID NO: 1668)
CAGGGUGGAUUUGGUAAUA

TDP43 AON is a gapmer oligonucleotide and has the following sequence and chemistry:

(SEQ ID NO: 1669)
5′ A*A*G*G*C*T*T*C*A*T*A*T*T*G*T*A*C*T*T*T 3′

where *=phosphorothioate, underlined=DNA, other=2′-MOE RNA: each “C” is 5-MeC.

To evaluate STMN2 AON ability to restore STMN2-FL, antisense oligonucleotides to STMN2 were co-incubated with TDP43 AON in RNAiMax. After 96 hours, transcript levels (e.g., STMN2 full length transcript, STMN2 transcript with cryptic exon, or TDP43 transcript) were detected by RT-qPCR using Taqman. Specifically, RT-qPCR was performed for detecting GAPDH using Thermofisher TaqMan Gene Expression Assay Hs03929097_g1. RT-qPCR was performed for detecting STMN2 transcripts with cryptic exon using the following primer sequences: 1) Forward primer: 5′-CTCAGTGCCTATCAGTCTCTC-3′ (SEQ ID NO: 1670), 2) Reverse primer: 5′-TCTTCTGCCGAGTCCCATT-3′ (SEQ ID NO: 1671) and 3) Probe: 5′-/56-FAM/TCAGCGTCTGCACATCCCTACAAT/3BHQ_1/-3′ (SEQ ID NO: 1672). RT-qPCR was performed for detecting full length STMN2 transcripts using the following primer sequences: 1) Forward primer: 5′-CCACGAACTTAGCTTCTCCA-3′ (SEQ ID NO: 1673), 2) Reverse primer: 5′-GCCAATGTTCAGCACCTG-3′ (SEQ ID NO: 1674), and 3)

Probe:
(SEQ ID NO: 1675)
5′-/56-FAM/ACTTTCTTCTTTCCTCTGCAGCCTCC/3BHQ_1/-3′.

RT-qPCR was performed on Applied Biosystems® 7500 Real-time PCR systems. One cycle of reverse transcription was performed at a temperature of 50° C. for 5 min. One cycle of RT inactivation/initial denaturation was performed at a temperature of 95° C. for 20 seconds. Forty five cycles of amplification were performed at a temperature of 95° C. for 1 second followed by 60° C. for 20 seconds.

STMN2-FL or STMN2 cryptic signal (Ct) was normalized to GAPDH (deltaCt). To visualize the quantitative changes (e.g., % increase of STMN-FL), the normalized STMN2-FL signal was further normalized to the vehicle (treated with RNAiMax alone, deltadeltaCt). Relative quantity of transcript level was calculated using the equation RQ=2^{-deltadeltaCt} and is used to describe the treatment condition comparison to normal, healthy levels (1.0).

Percent decrease of STMN2 with cryptic exon expression was calculated using the equation of:

$$100 - ( \frac{\begin{matrix}Mean relative quantity of STMN 2 with cryptic \\ exon in response to STMN 2 AON\end{matrix}}{\begin{matrix}Mean relative quantity of STMN 2 with cryptic \\ exon in response to TDP 43 AON\end{matrix}} * 100 )$$

The percent increase of full length STMN2 mRNA transcript was calculated using the equation of:

$$( ( \frac{\begin{matrix}Mean relative quantity of FL STMN 2 transcript \\ in response to STMN 2 AON\end{matrix}}{\begin{matrix}Mean relative quantity of FL STMN 2 transcript \\ in response to TDP 43 AON\end{matrix}} ) * 100 ) - 100$$

STMN2 antisense oligonucleotides were also evaluated in human motor neurons for potency in reducing cryptic exon and increasing STMN2 full length transcript. iCell human motor neurons (Cellular Dynamics International) were plated at 15×10³ cells/well in a 96-well plate for RT-qPCR RNA quantification or 3×10⁵ cells/well in a 6-well plate for western blot protein quantification according to manufacturer's instructions. Neurons were transfected with TDP43 AON and/or STMN2 AON using endoporter (GeneTools, LLC.) or treated with endoporter alone. Treatment conditions were tested in biological triplicate (qRT-PCR) or duplicate (western blot) wells. The same TDP43 AON described above is used here for evaluating human motor neurons. TDP43 AON is a gapmer oligonucleotide and has the following sequence and chemistry:

(SEQ ID NO: 1669)
5′ A*A*G*G*C*T*T*C*A*T*A*T*T*G*T*A*C*T*T*T 3′

where *=phosphorothioate, underlined=DNA, other=2′-MOE RNA.

After 72 hours, antisense oligonucleotides and endoporter were washed out and replaced with fresh media. After 72 additional hours, RNA was collected from the 96-well plates for RT-qPCR or protein collected from the 6-well plates for western blot. RNA was isolated, cDNA generated and multiplexed RT-qPCR assay performed with taqman probes for STMN2 cryptic exon, STMN2 full length transcript and reference GAPDH quantification. The same primers for detecting GAPDH, STMN2 transcript with cryptic exon, and full length STMN2, as described above in reference to SY5Y cells, were applied here for conducting RT-qPCR for human motor neurons. For protein quantification, the soluble portion of the protein collection was denatured and separated by SDS-PAGE, transferred to polyvinylidene difluoride membranes and probed with antibodies against GAPDH (Proteintech, 60004-1-1g), TDP-43 (Proteintech, 10782-2-AP), and Stathmin-2 (ThermoFisher, PA5-23049).

### Example 3: STMN2 Parent Oligonucleotides and Oligonucleotide Variants Restore Full Length STMN2 and Reduce STMN2 Transcripts with a Cryptic Exon

STMN2 parent oligonucleotides and oligonucleotide variants are tested for their ability to increase or restore full-length STMN2 mRNA (i.e., mRNA from which fill-length STMN2 is translated) levels in TDP43 silenced cells. In some cases, STMN2 oligonucleotides are tested for their ability to reduce STMN2 transcripts with a cryptic exon. As described further below, the quantified percentage increase/restoration of STMN2-FL and/or percentage reduction of STMN2 transcripts with cryptic exon is described in reference to levels of STMN-FL and/or STMN2 transcripts with cryptic exon in a control group (e.g., cells treated with 500 nM TDP43 AON).

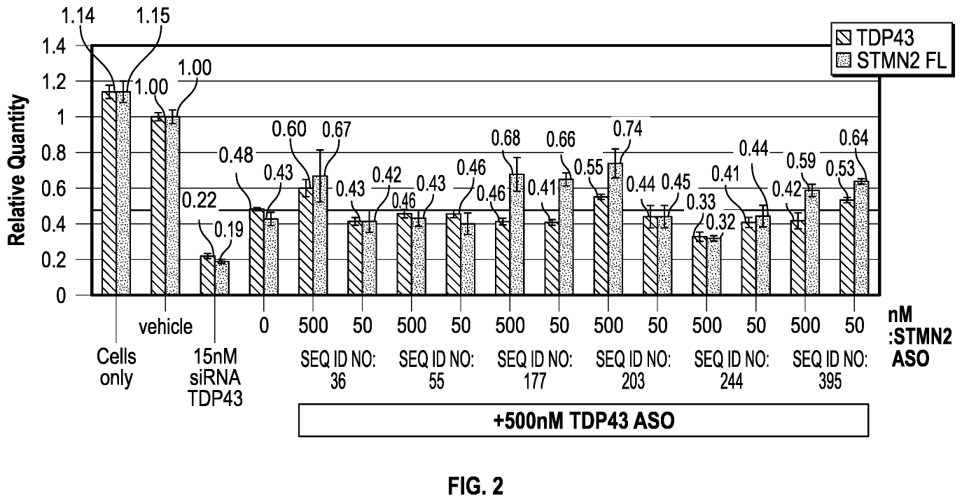

Referring to FIG. 2, TDP43 transcript was decreased by around 52% and STMN2-FL was decreased by around 57% when treated with 500 nM TDP43 AON. A 500 nM treatment of a STMN2 parent AON with SEQ ID NO: 36 increased TDP43 levels by 25% and increased STMN-FL levels by 55% (rescued to 67%). A 50 nM and a 500 nM treatment of a STMN2 parent oligonucleotide with SEQ ID NO: 177 increased STMN-FL levels by 58% (rescued to 66%) and 53% (rescued to 68%) respectively. A 500 nM treatment of a STMN2 parent oligonucleotide with SEQ ID NO: 203 increased TDP43 levels by 15% and STMN-FL levels by 72% (rescued to 74%). A 50 nM and a 500 nM treatment of a STMN2 parent oligonucleotide with SEQ ID NO: 395 increased STMN-FL levels by 49% (rescued to 64%) and 37% (rescued to 59%) respectively.

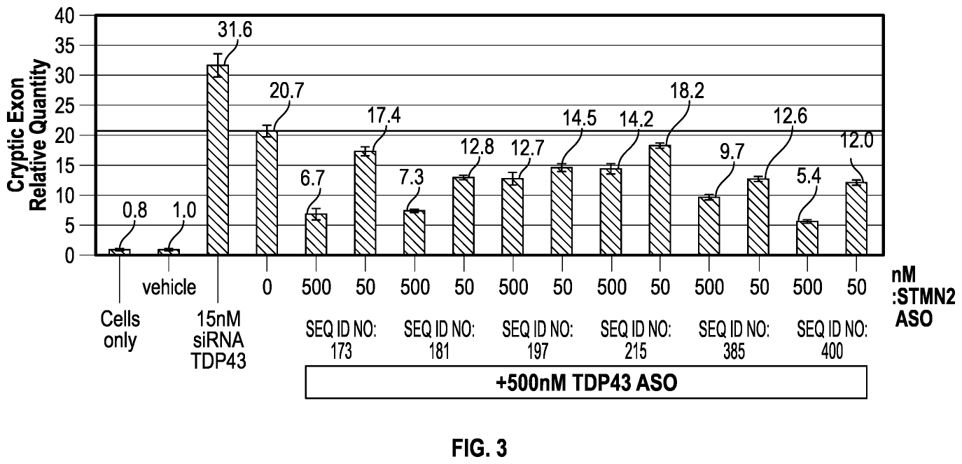

Referring to FIG. 3, the quantity of STMN2 transcript with cryptic exon was increased more than 20-fold when treated with 500 nM TDP43 AON. A 500 nM treatment of a STMN2 parent oligonucleotide with SEQ ID NO: 173 reduced STMN2 transcript with cryptic exon levels by 68%. A 500 nM treatment of a STMN2 parent oligonucleotide with SEQ ID NO: 181 reduced STMN2 transcript with cryptic exon levels by 65%. A 500 nM treatment of a STMN2 parent oligonucleotide with SEQ ID NO: 197 reduced STMN2 transcript with cryptic exon levels by 39%. A 500 nM treatment of a STMN2 parent oligonucleotide with SEQ ID NO: 215 reduced STMN2 transcript with cryptic exon levels by 31%. A 500 nM treatment of a STMN2 parent oligonucleotide with SEQ ID NO: 385 reduced STMN2 transcript with cryptic exon levels by 53%. A 500 nM treatment of a STMN2 parent oligonucleotide with SEQ ID NO: 400 reduced STMN2 transcript with cryptic exon levels by 74%.

Referring to FIG. 4, STMN2-FL was decreased by around 59% when treated with 500 nM TDP43 AON. A 500 nM treatment of a STMN2 parent oligonucleotide with SEQ ID NO: 173 increased STMN-FL levels by 66% (rescued to 68%). A 500 nM treatment of a STMN2 parent oligonucleotide with SEQ ID NO: 197 increased STMN-FL levels by 46% (rescued to 60%).

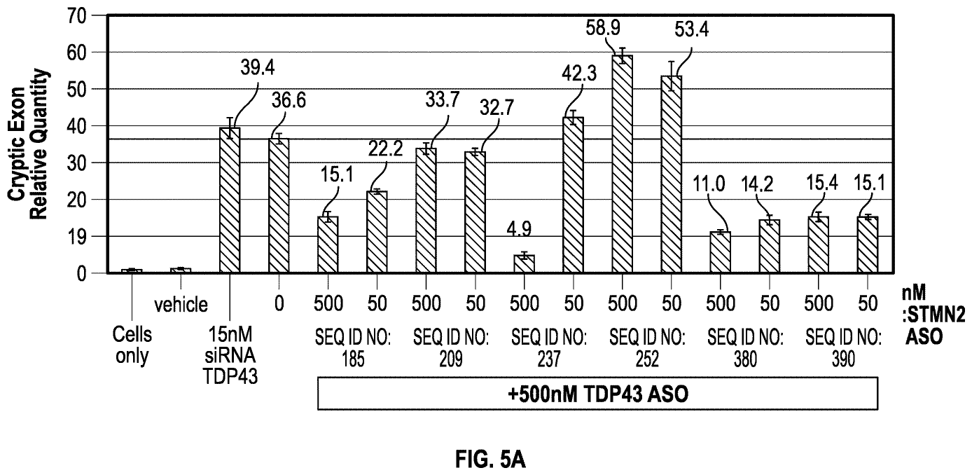

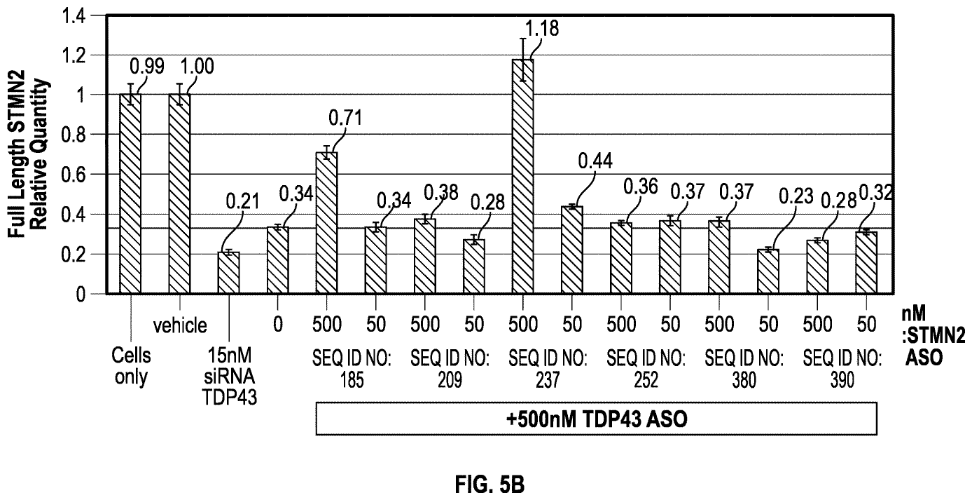

Referring to FIG. 5A, the quantity of STMN2 transcript with cryptic exon was increased more than 36-fold when treated with 500 nM TDP43 AON. A 500 nM treatment of a STMN2 parent oligonucleotide with SEQ ID NO: 185 reduced STMN2 transcript with cryptic exon levels by 58%. A 500 nM treatment of a STMN2 parent oligonucleotide with SEQ ID NO: 237 reduced STMN2 transcript with cryptic exon levels by 87%. A 500 nM treatment of a STMN2 parent oligonucleotide with SEQ ID NO: 380 reduced STMN2 transcript with cryptic exon levels by 70%. A 500 nM treatment of a STMN2 parent oligonucleotide with SEQ ID NO: 390 reduced STMN2 transcript with cryptic exon levels by 58%.

Referring to FIG. 5B, STMN2-FL was decreased by 66% when treated with 500 nM TDP43 AON. A 500 nM treatment of a STMN2 parent oligonucleotide with SEQ ID NO: 185 increased STMN-FL levels by 109% (rescued to 71%). A 500 nM treatment of a STMN2 parent oligonucleotide with SEQ ID NO: 237 increased STMN-FL levels by 247% (rescued to 118%).

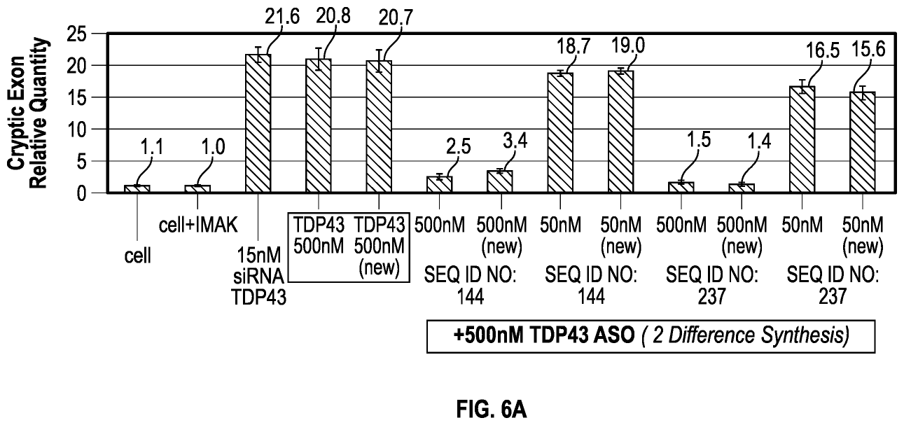

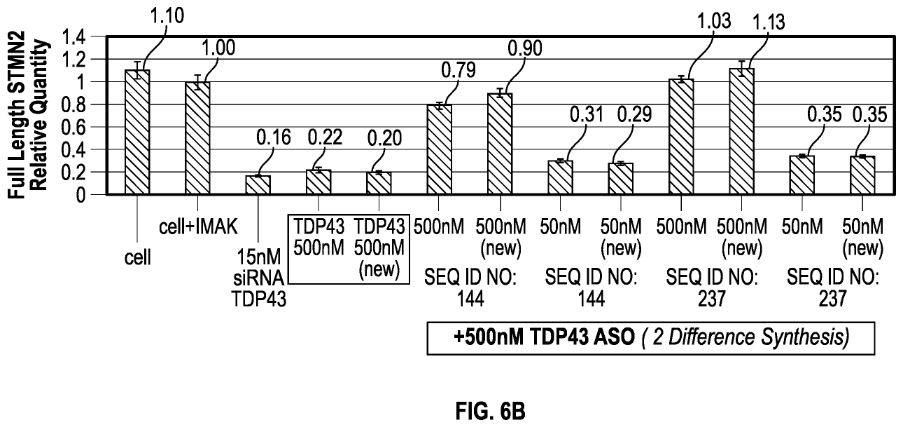

Referring to FIG. 6A, the quantity of STMN2 transcript with cryptic exon was increased more than 20-fold when treated with 500 nM TDP43 AON (two different syntheses). A 500 nM treatment of a STMN2 parent oligonucleotide with SEQ ID NO: 144 reduced STMN2 transcript with cryptic exon levels by 83 to 88%. A 500 nM treatment of a STMN2 parent oligonucleotide with SEQ ID NO: 237 reduced STMN2 transcript with cryptic exon levels by 92 to 93%.

Referring to FIG. 6B, STMN2-FL was decreased by about 80% when treated with 500 nM TDP43 AON. A 500 nM treatment of a STMN2 parent oligonucleotide with SEQ ID NO: 144 increased STMN-FL levels by 276% to 329% (rescued to 79% to 90%). A 500 nM treatment of a STMN2 parent oligonucleotide with SEQ ID NO: 237 increased STMN-FL levels by 390% to 438% (rescued to 103% to 113%).

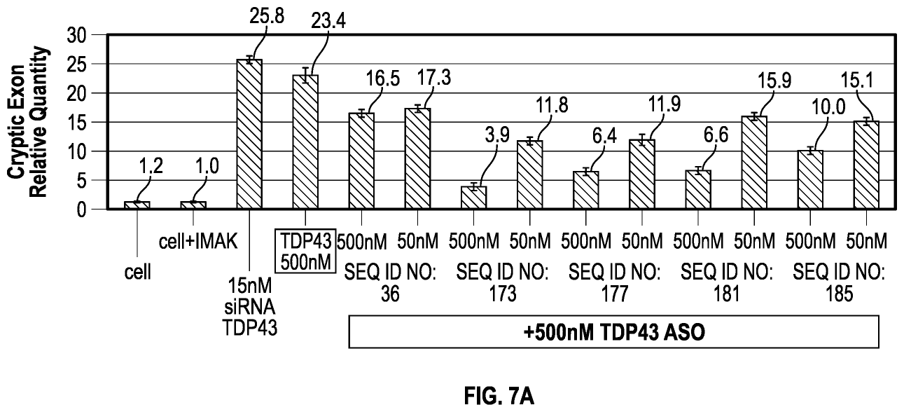

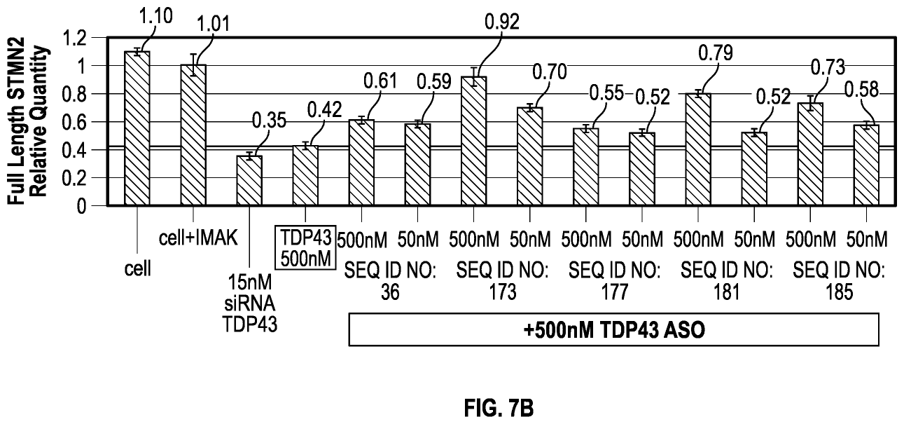

Referring to FIG. 7A, the quantity of STMN2 transcript with cryptic exon was increased more than 23-fold when treated with 500 nM TDP43 AON. A 500 nM treatment of a STMN2 parent oligonucleotide with SEQ ID NO: 173 reduced STMN2 transcript with cryptic exon levels by 83%. A 500 nM treatment of a STMN2 parent oligonucleotide with SEQ ID NO: 177 reduced STMN2 transcript with cryptic exon levels by 83%. A 500 nM treatment of a STMN2 parent oligonucleotide with SEQ ID NO: 181 reduced STMN2 transcript with cryptic exon levels by 72%.

Referring to FIG. 7B, STMN2-FL was decreased by about 58% when treated with 500 nM TDP43 AON. A 500 nM treatment of a STMN2 parent oligonucleotide with SEQ ID NO: 173 increased STMN-FL levels by 119% (rescued to 92%). A 500 nM treatment of a STMN2 parent oligonucleotide with SEQ ID NO: 181 increased STMN-FL levels by 88% (rescued to 79%). A 500 nM treatment of a STMN2 parent oligonucleotide with SEQ ID NO: 185 increased STMN-FL levels by 74% (rescued to 73%).

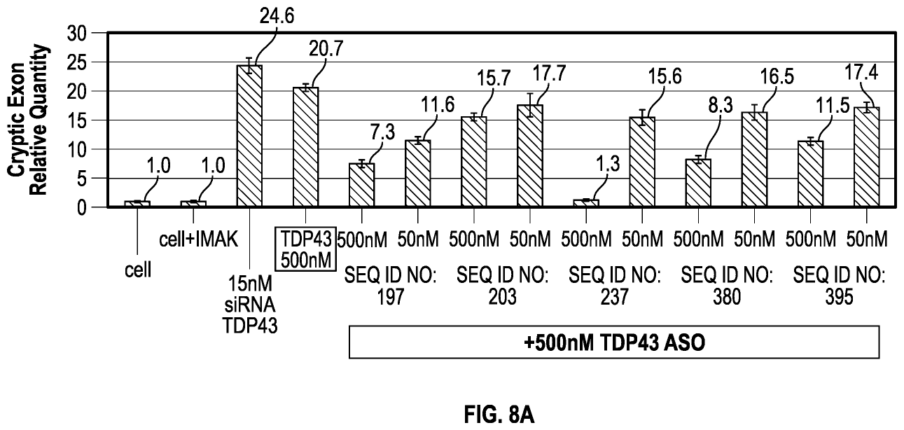

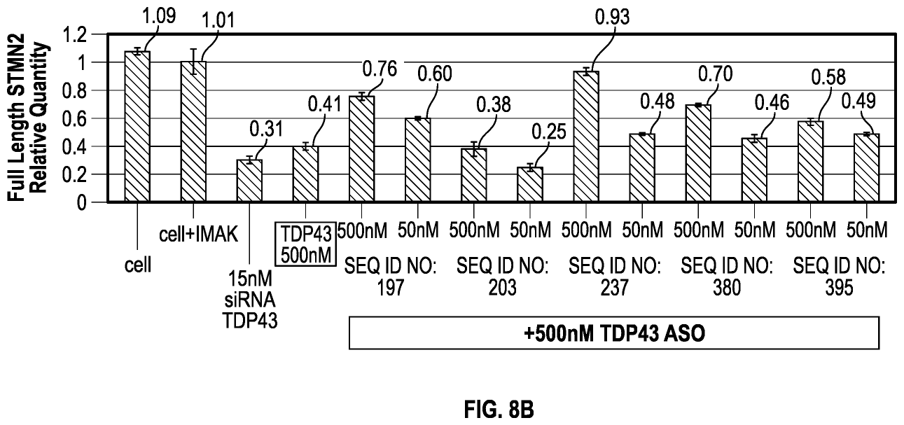

Referring to FIG. 8A, the quantity of STMN2 transcript with cryptic exon was increased more than 20-fold when treated with 500 nM TDP43 AON. A 500 nM treatment of a STMN2 parent oligonucleotide with SEQ ID NO: 197 reduced STMN2 transcript with cryptic exon levels by 65%. A 500 nM treatment of a STMN2 parent oligonucleotide with SEQ ID NO: 237 reduced STMN2 transcript with cryptic exon levels by 94%.

Referring to FIG. 8B, STMN2-FL was decreased by 59% when treated with 500 nM TDP43 AON. A 500 nM treatment of a STMN2 parent oligonucleotide with SEQ ID NO: 197 increased STMN-FL levels by 85% (rescued to 76%). A 500 nM treatment of a STMN2 parent oligonucleotide with SEQ ID NO: 237 increased STMN-FL levels by 127% (rescued to 93%). A 500 nM treatment of a STMN2 parent oligonucleotide with SEQ ID NO: 380 increased STMN-FL levels by 71% (rescued to 70%).

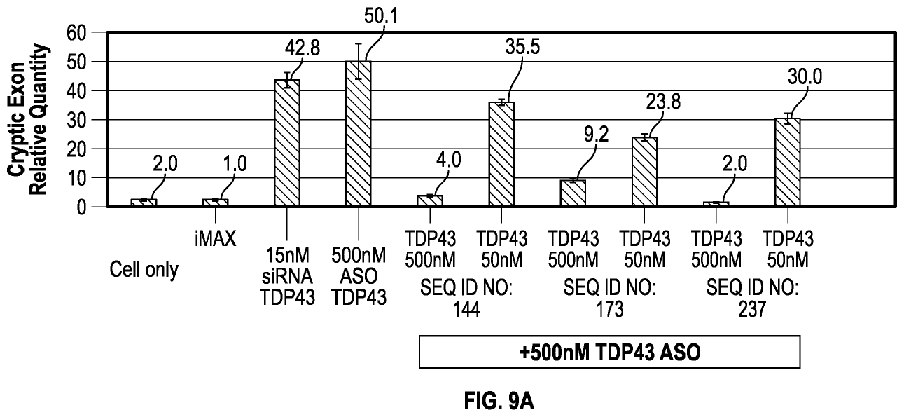

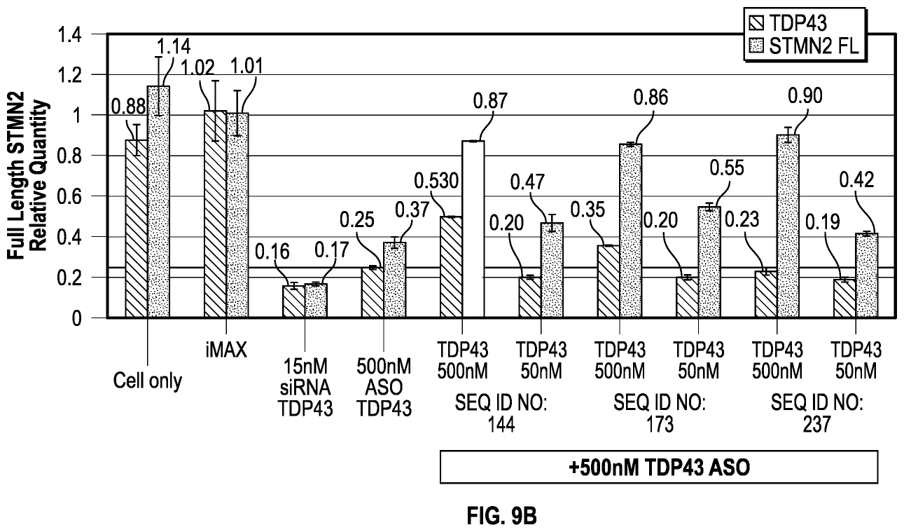

Referring to FIG. 9A, the quantity of STMN2 transcript with cryptic exon was increased more than 50-fold when treated with 500 nM TDP43 AON. A 500 nM treatment of a STMN2 parent oligonucleotide with SEQ ID NO: 144 reduced STMN2 transcript with cryptic exon levels by 92%. A 500 nM treatment of a STMN2 parent oligonucleotide with SEQ ID NO: 173 reduced STMN2 transcript with cryptic exon levels by 82%. A 500 nM treatment of a STMN2 parent oligonucleotide with SEQ ID NO: 237 reduced STMN2 transcript with cryptic exon levels by 96%.

Referring to FIG. 9B, STMN2-FL was decreased by 67% when treated with 500 nM TDP43 AON. A 500 nM treatment of a STMN2 parent oligonucleotide with SEQ ID NO: 144 increased STMN-FL levels by 135% (rescued to 87%). A 500 nM treatment of a STMN2 parent oligonucleotide with SEQ ID NO: 173 increased STMN-FL levels by 132% (rescued to 86%). A 500 nM treatment of a STMN2 parent oligonucleotide with SEQ ID NO: 237 increased STMN-FL levels by 143% (rescued to 90%).

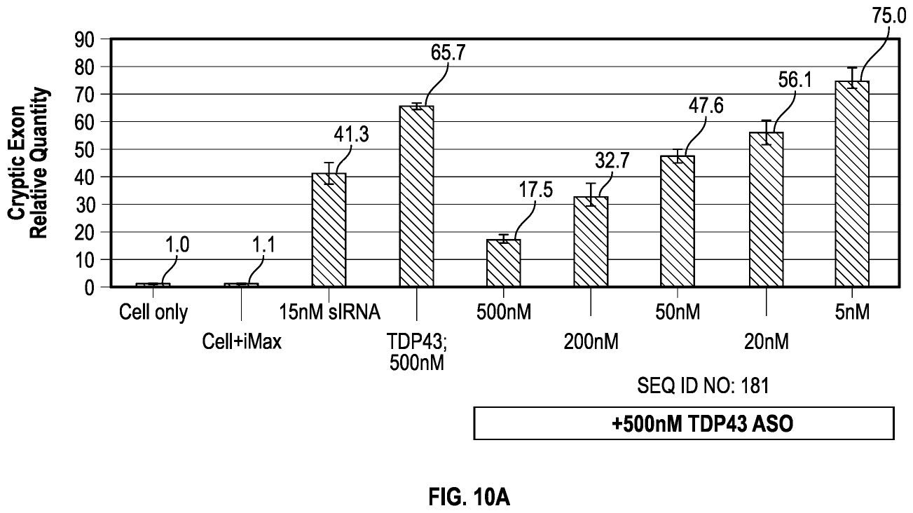

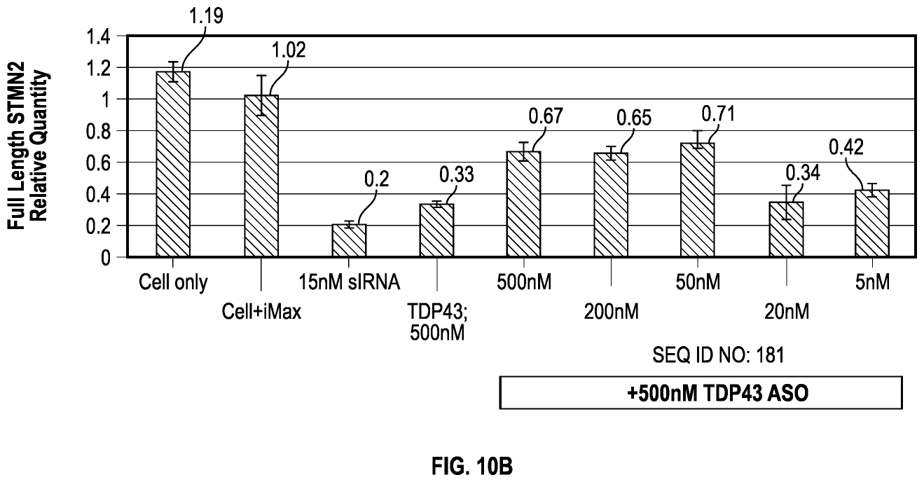

Referring to FIG. 10A, the quantity of STMN2 transcript with cryptic exon was increased more than 65-fold when treated with 500 nM TDP43 AON. A 200 nM treatment of a STMN2 parent oligonucleotide with SEQ ID NO: 181 reduced STMN2 transcript with cryptic exon levels by 50%. A 500 nM treatment of a STMN2 parent oligonucleotide with SEQ ID NO: 181 reduced STMN2 transcript with cryptic exon levels by 73%. Referring to FIG. 10B, STMN2-FL was decreased by 67% when treated with 500 nM TDP43 AON. A 50 nM treatment of a STMN2 parent oligonucleotide with SEQ ID NO: 181 increased STMN-FL levels by 115% (rescued to 71%). A 200 nM treatment of a STMN2 parent oligonucleotide with SEQ ID NO: 181 increased STMN-FL levels by 97% (rescued to 65%). A 500 nM treatment of a STMN2 parent oligonucleotide with SEQ ID NO: 181 increased STMN-FL levels by 94% (rescued to 64%).

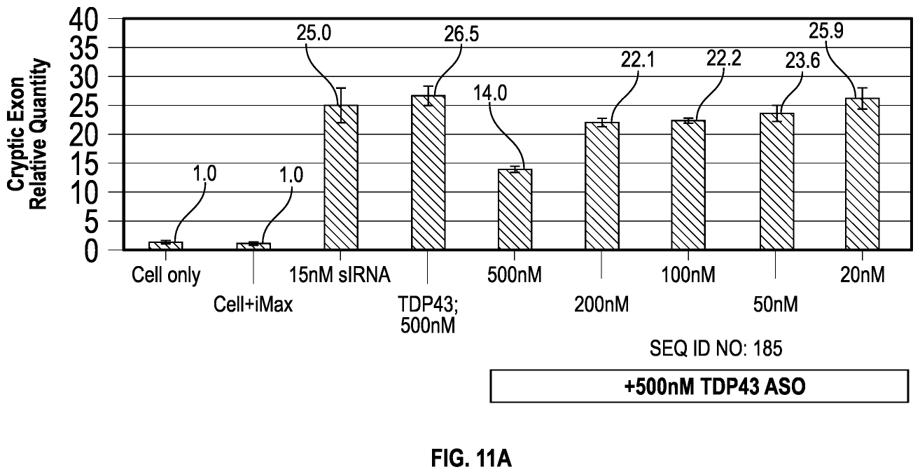

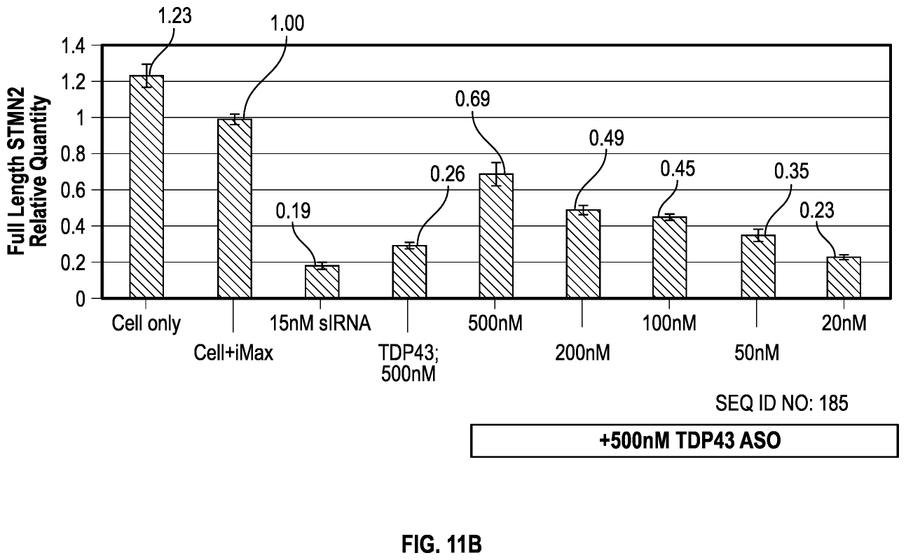

Referring to FIG. 11A, the quantity of STMN2 transcript with cryptic exon was increased more than 26-fold when treated with 500 nM TDP43 AON. A 500 nM treatment of a STMN2 parent oligonucleotide with SEQ ID NO: 185 reduced STMN2 transcript with cryptic exon levels by 47%. Referring to FIG. 11B, STMN2-FL was decreased by 74% when treated with 500 nM TDP43 AON. A 50 nM treatment of a STMN2 parent oligonucleotide with SEQ ID NO: 185 increased STMN-FL levels by 73% (rescued to 45%). A 200 nM treatment of a STMN2 parent oligonucleotide with SEQ ID NO: 185 increased STMN-FL levels by 246% (rescued to 90%). A 500 nM treatment of a STMN2 parent oligonucleotide with SEQ ID NO: 185 increased STMN-FL levels by 165% (rescued to 69%).

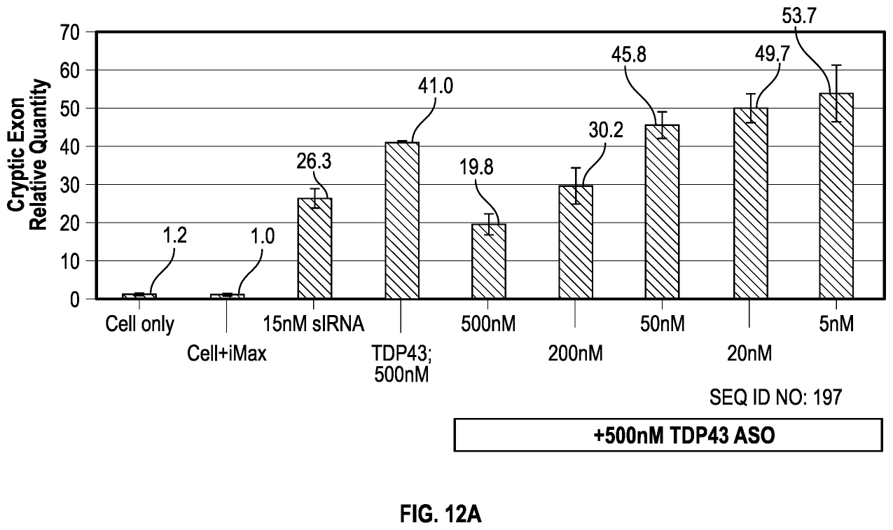

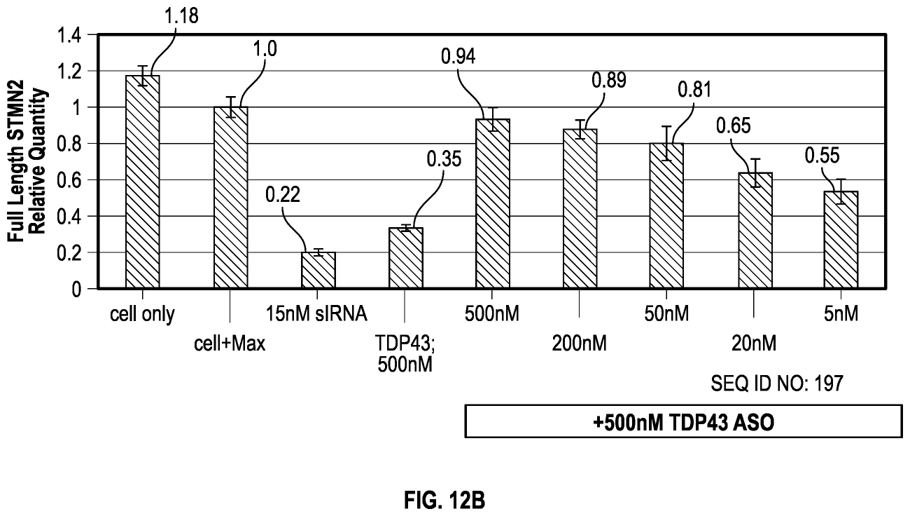

Referring to FIG. 12A, the quantity of STMN2 transcript with cryptic exon was increased more than 41-fold when treated with 500 nM TDP43 AON. A 500 nM treatment of a STMN2 parent oligonucleotide with SEQ ID NO: 197 reduced STMN2 transcript with cryptic exon levels by 51%. Referring to FIG. 12B, STMN2-FL was decreased by 65% when treated with 500 nM TDP43 AON. A 20 nM treatment of a STMN2 parent oligonucleotide with SEQ ID NO: 197 increased STMN-FL levels by 86% (rescued to 65%). A 50 nM treatment of a STMN2 parent oligonucleotide with SEQ ID NO: 197 increased STMN-FL levels by 131% (rescued to 81%). A 200 nM treatment of a STMN2 parent oligonucleotide with SEQ ID NO: 197 increased STMN-FL levels by 154% (rescued to 89%). A 500 nM treatment of a STMN2 parent oligonucleotide with SEQ ID NO: 197 increased STMN-FL levels by 169% (rescued to 94%).

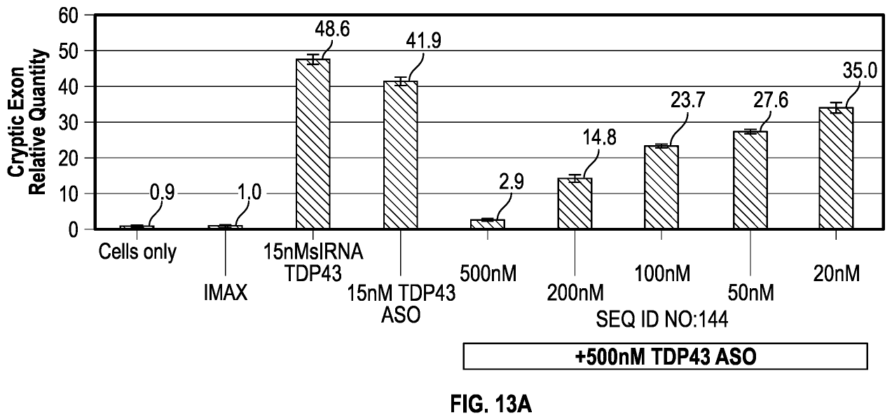

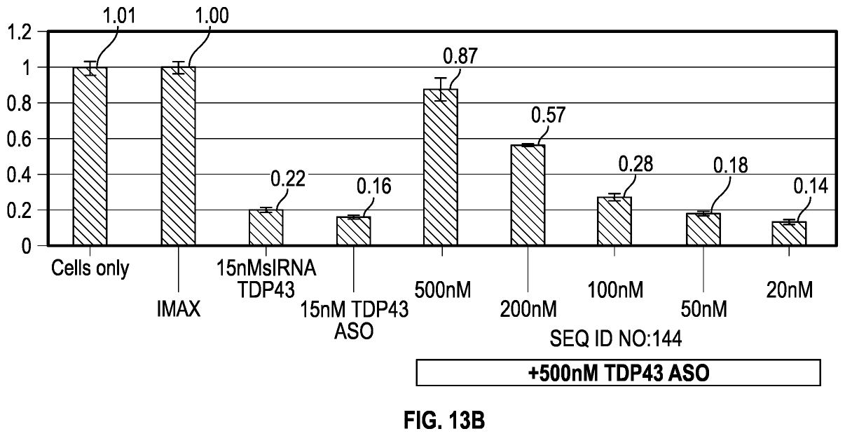

Referring to FIG. 13A, the quantity of STMN2 transcript with cryptic exon was increased more than 41-fold when treated with 500 nM TDP43 AON. A 500 nM treatment of a STMN2 parent oligonucleotide with SEQ ID NO: 144 reduced STMN2 transcript with cryptic exon levels by 93%. Referring to FIG. 13B, STMN2-FL was decreased by 84% when treated with 500 nM TDP43 AON. A 50 nM treatment of a STMN2 parent oligonucleotide with SEQ ID NO: 144 increased STMN-FL levels by 75% (rescued to 28%). A 200 nM treatment of a STMN2 parent oligonucleotide with SEQ ID NO: 144 increased STMN-FL levels by 260% (rescued to 57%). A 500 nM treatment of a STMN2 parent oligonucleotide with SEQ ID NO: 144 increased STMN-FL levels by 444% (rescued to 87%).

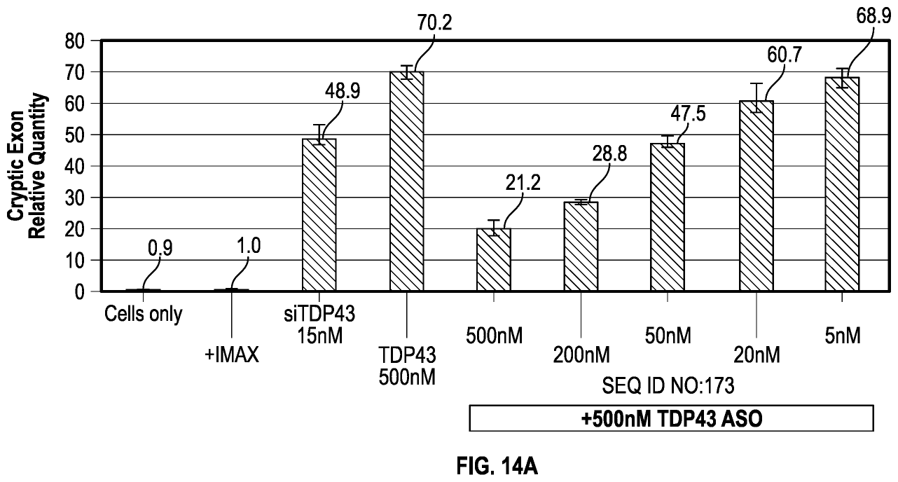

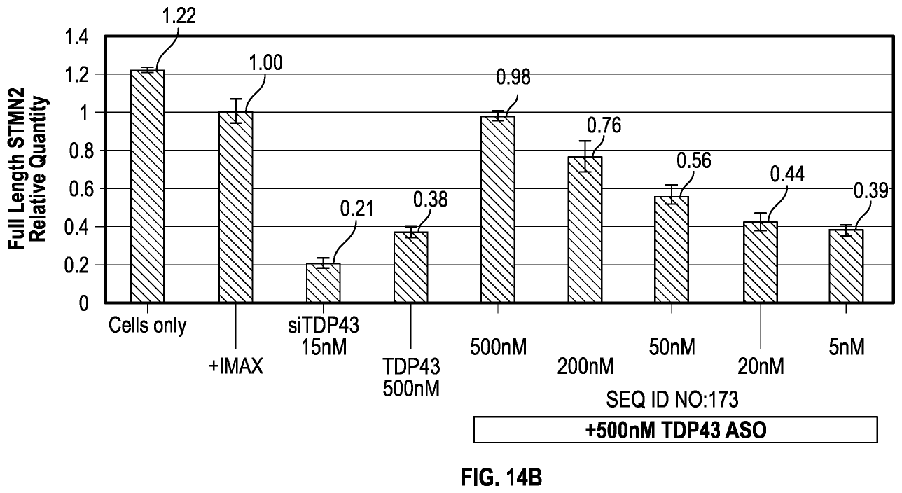

Referring to FIG. 14A, the quantity of STMN2 transcript with cryptic exon was increased more than 24-fold when treated with 500 nM TDP43 AON. A 200 nM treatment of a STMN2 parent oligonucleotide with SEQ ID NO: 173 reduced STMN2 transcript with cryptic exon levels by 59%. A 500 nM treatment of a STMN2 parent oligonucleotide with SEQ ID NO: 173 reduced STMN2 transcript with cryptic exon levels by 70%. Referring to FIG. 14B, STMN2-FL was decreased by 62% when treated with 500 nM TDP43 AON. A 200 nM treatment of a STMN2 parent oligonucleotide with SEQ ID NO: 173 increased STMN-FL levels by 100% (rescued to 76%). A 500 nM treatment of a STMN2 parent oligonucleotide with SEQ ID NO: 173 increased STMN-FL levels by 158% (rescued to 98%).

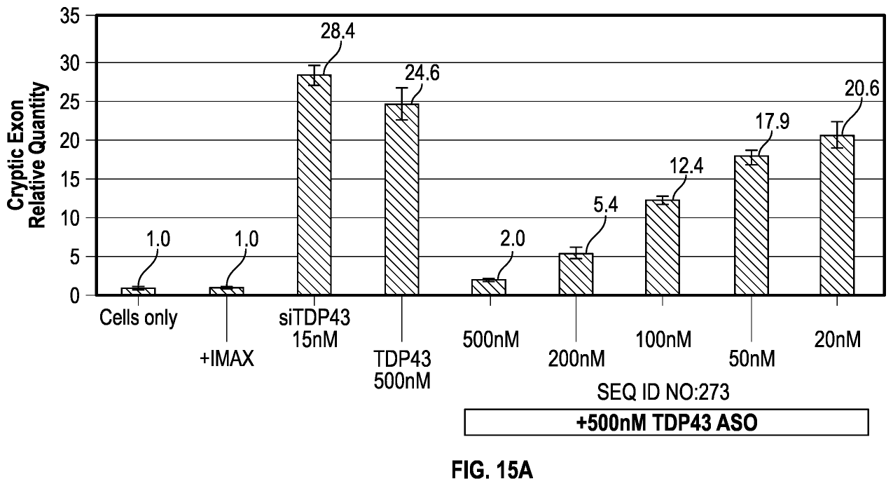

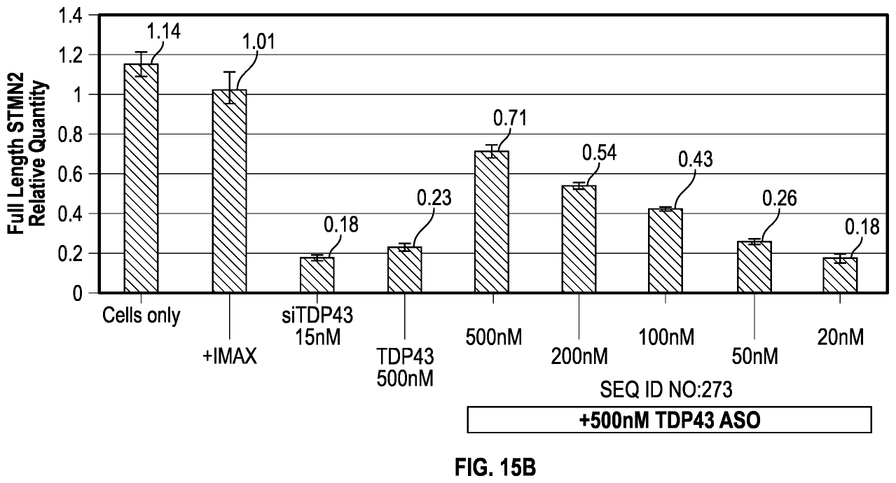

Referring to FIG. 15A, the quantity of STMN2 transcript with cryptic exon was increased more than 70-fold when treated with 500 nM TDP43 AON. A 200 nM treatment of a STMN2 parent oligonucleotide with SEQ ID NO: 237 reduced STMN2 transcript with cryptic exon levels by 78%. A 500 nM treatment of a STMN2 parent oligonucleotide with SEQ ID NO: 237 reduced STMN2 transcript with cryptic exon levels by 92%. Referring to FIG. 15B, STMN2-FL was decreased by 77% when treated with 500 nM TDP43 AON. A 50 nM treatment of a STMN2 parent oligonucleotide with SEQ ID NO: 237 increased STMN-FL levels by 87% (rescued to 43%). A 200 nM treatment of a STMN2 parent oligonucleotide with SEQ ID NO: 237 increased STMN-FL levels by 135% (rescued to 54%). A 500 nM treatment of a STMN2 parent oligonucleotide with SEQ ID NO: 237 increased STMN-FL levels by 209% (rescued to 71%).

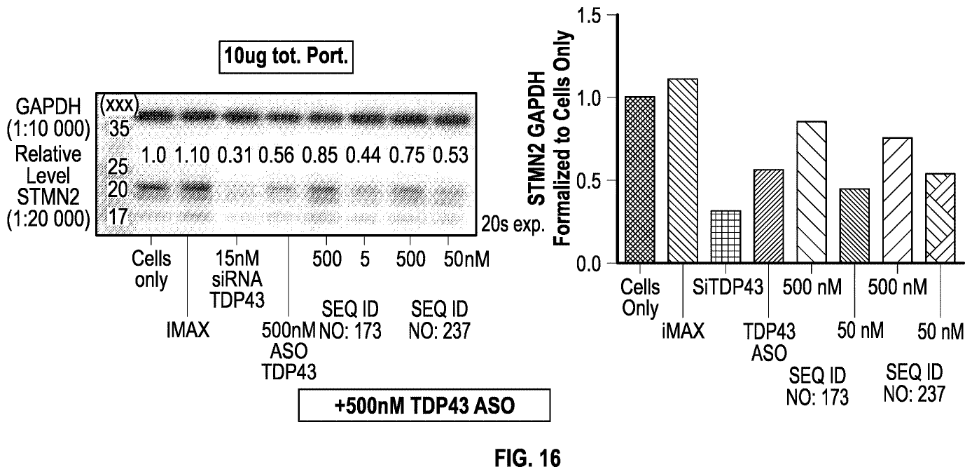

Referring to FIG. 16, STMN2 protein levels were decreased by 44% when treated with 500 nM TDP43 AON. A 500 nM treatment of a STMN2 parent oligonucleotide with SEQ ID NO: 173 increased STMN protein levels by 52%. A 500 nM treatment of a STMN2 parent oligonucleotide with SEQ ID NO: 237 increased STMN protein levels by 34%.

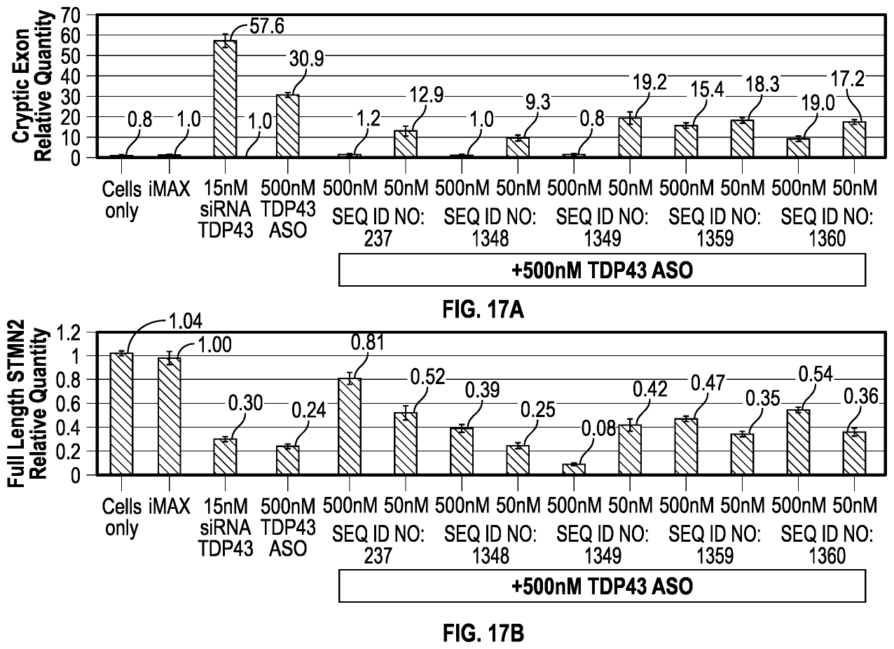

Referring to FIG. 17A, the quantity of STMN2 transcript with cryptic exon was increased more than 30-fold when treated with 500 nM TDP43 AON. A 500 nM treatment of a STMN2 parent oligonucleotide with SEQ ID NO: 237 reduced STMN2 transcript with cryptic exon levels by 96%. A 500 nM treatment of a STMN2 oligonucleotide variant with SEQ ID NO: 1348 reduced STMN2 transcript with cryptic exon levels by 97%. A 500 nM treatment of a STMN2 oligonucleotide variant with SEQ ID NO: 1349 reduced STMN2 transcript with cryptic exon levels by 97%. A 500 nM treatment of a STMN2 oligonucleotide variant with SEQ ID NO: 1360 reduced STMN2 transcript with cryptic exon levels by 71%.

Referring to FIG. 17B, STMN2-FL was decreased by 76% when treated with 500 nM TDP43 AON. A 500 nM treatment of a STMN2 parent oligonucleotide with SEQ ID NO: 237 increased STMN-FL levels by 238% (rescued to 81%). A 500 nM treatment of a STMN2 oligonucleotide variant with SEQ ID NO: 1348 increased STMN-FL levels by 63% (rescued to 39%). A 500 nM treatment of a STMN2 oligonucleotide variant with SEQ ID NO: 1359 increased STMN-FL levels by 96% (rescued to 47%). A 500 nM treatment of a STMN2 oligonucleotide variant with SEQ ID NO: 1360 increased STMN-FL levels by 125% (rescued to 54%).

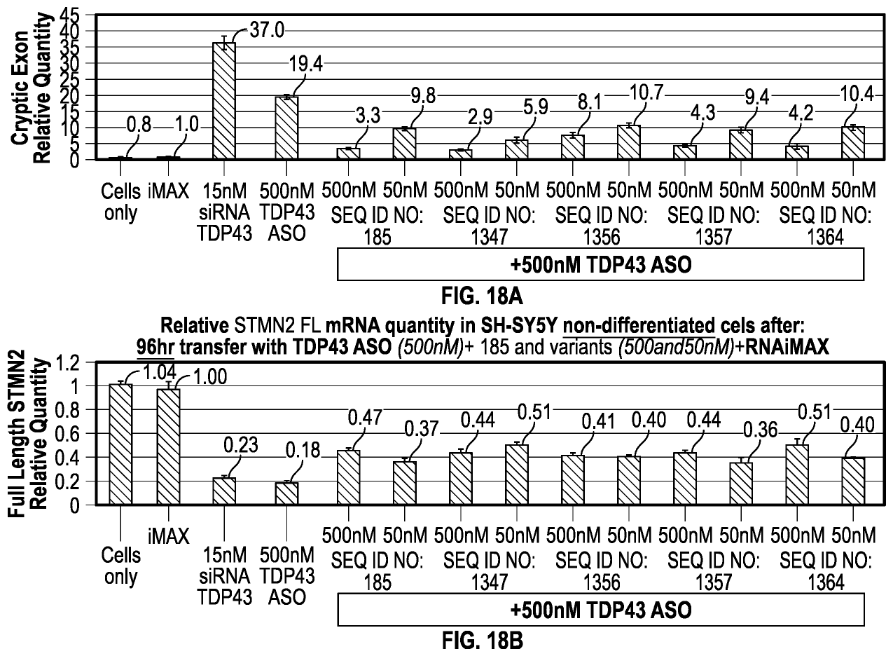

Referring to FIG. 18A, the quantity of STMN2 transcript with cryptic exon was increased more than 19-fold when treated with 500 nM TDP43 AON. A 500 nM treatment of a STMN2 parent oligonucleotide with SEQ ID NO: 185 reduced STMN2 transcript with cryptic exon levels by 83%. A 500 nM treatment of a STMN2 oligonucleotide variant with SEQ ID NO: 1347 reduced STMN2 transcript with cryptic exon levels by 85%. A 500 nM treatment of a STMN2 oligonucleotide variant with SEQ ID NO: 1356 reduced STMN2 transcript with cryptic exon levels by 56%. A 500 nM treatment of a STMN2 oligonucleotide variant with SEQ ID NO: 1357 reduced STMN2 transcript with cryptic exon levels by 78%. A 500 nM treatment of a STMN2 oligonucleotide variant with SEQ ID NO: 1364 reduced STMN2 transcript with cryptic exon levels by 78%.

Referring to FIG. 18B, STMN2-FL was decreased by 82% when treated with 500 nM TDP43 AON. A 500 nM treatment of a STMN2 parent oligonucleotide with SEQ ID NO: 185 increased STMN-FL levels by 161% (rescued to 47%). A 500 nM treatment of a STMN2 oligonucleotide variant with SEQ ID NO: 1347 increased STMN-FL levels by 144% (rescued to 44%). A 500 nM treatment of a STMN2 oligonucleotide variant with SEQ ID NO: 1356 increased STMN-FL levels by 128% (rescued to 41%). A 500 nM treatment of a STMN2 oligonucleotide variant with SEQ ID NO: 1357 increased STMN-FL levels by 144% (rescued to 44%). A 500 nM treatment of a STMN2 oligonucleotide variant with SEQ ID NO: 1364 increased STMN-FL levels by 183% (rescued to 51%).

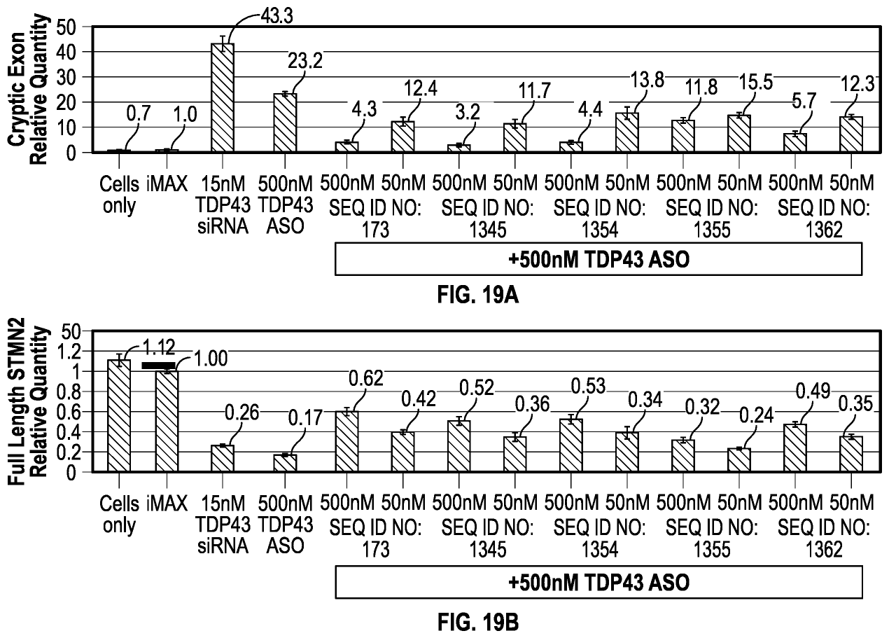

Referring to FIG. 19A, the quantity of STMN2 transcript with cryptic exon was increased more than 23-fold when treated with 500 nM TDP43 AON. A 500 nM treatment of a STMN2 parent oligonucleotide with SEQ ID NO: 173 reduced STMN2 transcript with cryptic exon levels by 81%. A 500 nM treatment of a STMN2 oligonucleotide variant with SEQ ID NO: 1345 reduced STMN2 transcript with cryptic exon levels by 86%. A 500 nM treatment of a STMN2 oligonucleotide variant with SEQ ID NO: 1354 reduced STMN2 transcript with cryptic exon levels by 81%. A 500 nM treatment of a STMN2 oligonucleotide variant with SEQ ID NO: 1355 reduced STMN2 transcript with cryptic exon levels by 47%. A 500 nM treatment of a STMN2 oligonucleotide variant with SEQ ID NO: 1362 reduced STMN2 transcript with cryptic exon levels by 75%.

Referring to FIG. 19B, STMN2-FL was decreased by 83% when treated with 500 nM TDP43 AON. A 500 nM treatment of a STMN2 parent oligonucleotide with SEQ ID NO: 173 increased STMN-FL levels by 265% (rescued to 62%). A 500 nM treatment of a STMN2 oligonucleotide variant with SEQ ID NO: 1345 increased STMN-FL levels by 206% (rescued to 52%). A 500 nM treatment of a STMN2 oligonucleotide variant with SEQ ID NO: 1354 increased STMN-FL levels by 212% (rescued to 53%). A 500 nM treatment of a STMN2 oligonucleotide variant with SEQ ID NO: 1355 increased STMN-FL levels by 88% (rescued to 32%). A 500 nM treatment of a STMN2 oligonucleotide variant with SEQ ID NO: 1362 increased STMN-FL levels by 188% (rescued to 49%).

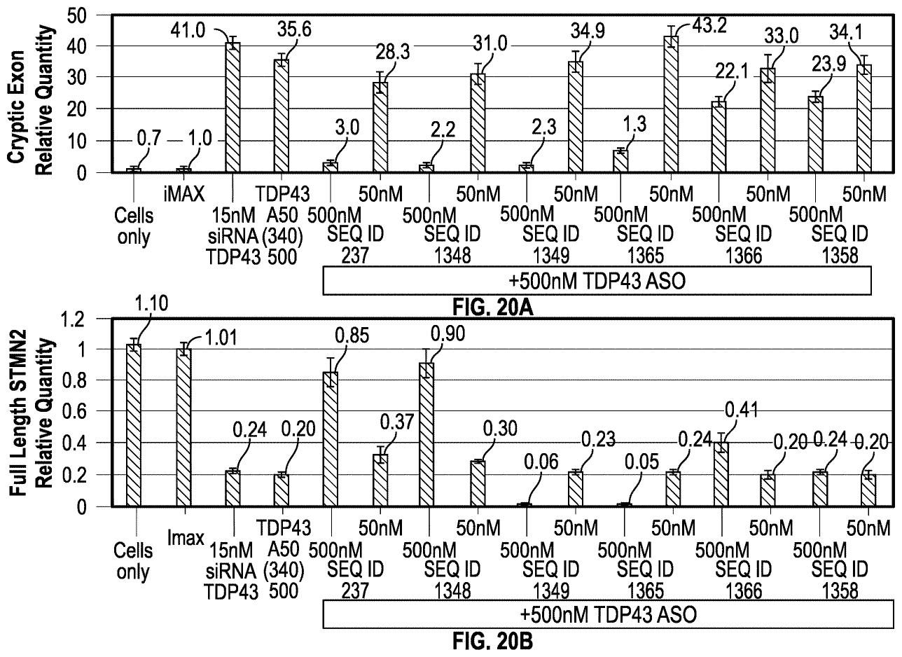

Referring to FIG. 20A, the quantity of STMN2 transcript with cryptic exon was increased more than 35-fold when treated with 500 nM TDP43 AON. A 500 nM treatment of a STMN2 parent oligonucleotide with SEQ ID NO: 237 reduced STMN2 transcript with cryptic exon levels by 91%. A 500 nM treatment of a STMN2 oligonucleotide variant with SEQ ID NO: 1348 reduced STMN2 transcript with cryptic exon levels by 94%. A 500 nM treatment of a STMN2 oligonucleotide variant with SEQ ID NO: 1349 reduced STMN2 transcript with cryptic exon levels by 96%. A 500 nM treatment of a STMN2 oligonucleotide variant with SEQ ID NO: 1365 reduced STMN2 transcript with cryptic exon levels by 82%. A 500 nM treatment of a STMN2 oligonucleotide variant with SEQ ID NO: 1366 reduced STMN2 transcript with cryptic exon levels by 38%. A 500 nM treatment of a STMN2 oligonucleotide variant with SEQ ID NO: 1358 reduced STMN2 transcript with cryptic exon levels by 33%.

Referring to FIG. 20B, STMN2-FL was decreased by 80% when treated with 500 nM TDP43 AON. A 500 nM treatment of a STMN2 parent oligonucleotide with SEQ ID NO: 237 increased STMN-FL levels by 325% (rescued to 85%). A 500 nM treatment of a STMN2 oligonucleotide variant with SEQ ID NO: 1348 increased STMN-FL levels by 350% (rescued to 90%). A 500 nM treatment of a STMN2 oligonucleotide variant with SEQ ID NO: 1366 increased STMN-FL levels by 105% (rescued to 41%). A 500 nM treatment of a STMN2 oligonucleotide variant with SEQ ID NO: 1358 increased STMN-FL levels by 20% (rescued to 24%).

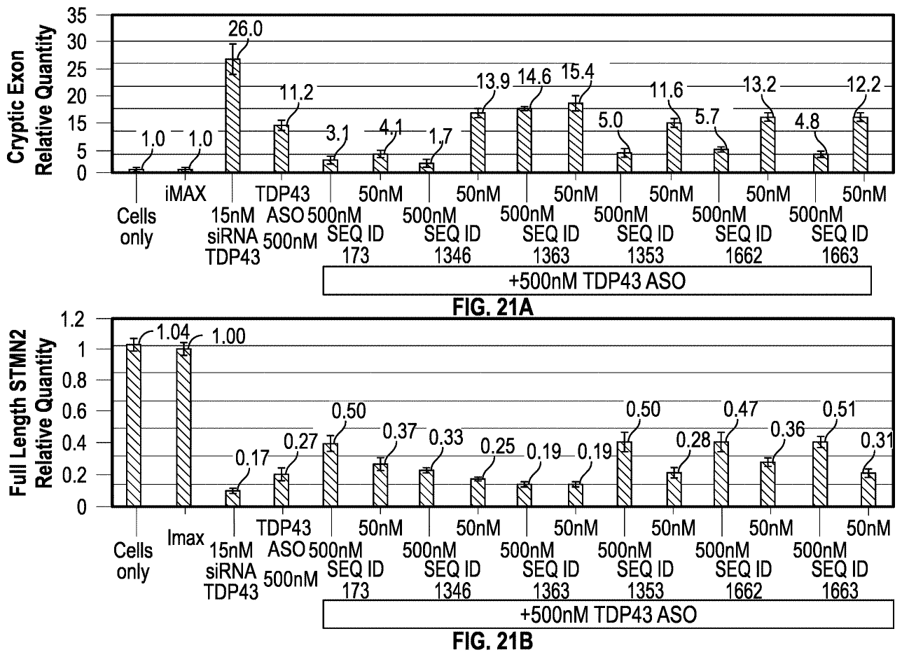

Referring to FIG. 21A, the quantity of STMN2 transcript with cryptic exon was increased more than 11-fold when treated with 500 nM TDP43 AON. A 500 nM treatment of a STMN2 parent oligonucleotide with SEQ ID NO: 173 reduced STMN2 transcript with cryptic exon levels by 72%. A 500 nM treatment of a STMN2 oligonucleotide variant with SEQ ID NO: 1346 reduced STMN2 transcript with cryptic exon levels by 85%. A 500 nM treatment of a STMN2 oligonucleotide variant with SEQ ID NO: 1353 reduced STMN2 transcript with cryptic exon levels by 55%. A 500 nM treatment of a STMN2 oligonucleotide variant with SEQ ID NO: 1662 (G*A*G*TCCTGCAATATGAATATA*AT*T*T, where * indicates phosphodiester linkage) reduced STMN2 transcript with cryptic exon levels by 49%. A 500 nM treatment of a STMN2 oligonucleotide variant with SEQ ID NO: 1663 (GAGTCCTG*C*A*A*T*A*TGAATATAATTT, where * indicates phosphodiester linkage) reduced STMN2 transcript with cryptic exon levels by 57%.

Referring to FIG. 21B, STMN2-FL was decreased by 73% when treated with 500 nM TDP43 AON. A 500 nM treatment of a STMN2 parent oligonucleotide with SEQ ID NO: 173 increased STMN-FL levels by 85% (rescued to 50%). A 500 nM treatment of a STMN2 oligonucleotide variant with SEQ ID NO: 1353 increased STMN-FL levels by 85% (rescued to 50%). A 500 nM treatment of a STMN2 oligonucleotide variant with SEQ ID NO: 1662 increased STMN-FL levels by 74% (rescued to 47%). A 500 nM treatment of a STMN2 oligonucleotide variant SEQ ID NO: 1663 increased STMN-FL levels by 89% (rescued to 51%).

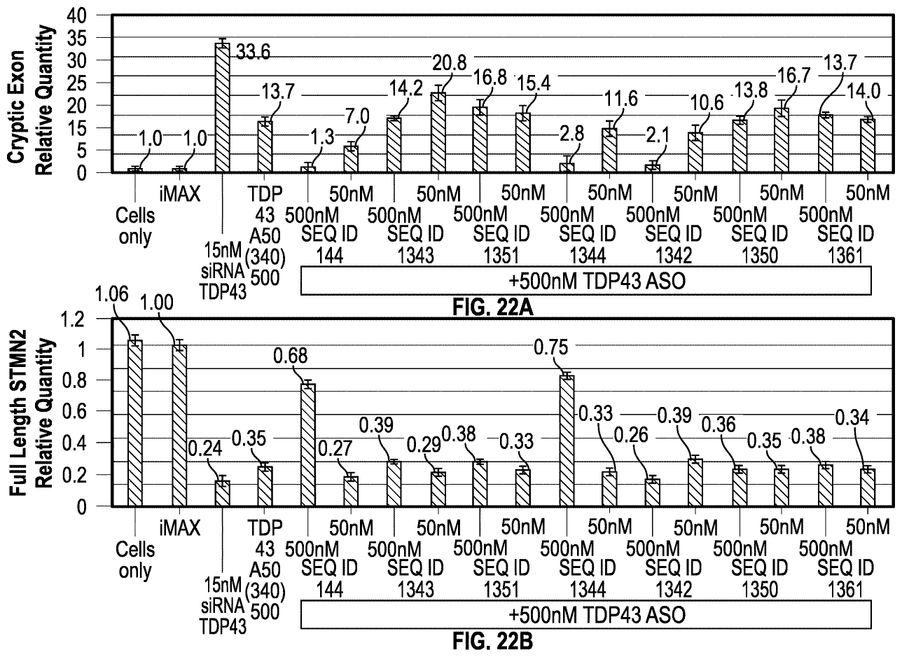

Referring to FIG. 22A, the quantity of STMN2 transcript with cryptic exon was increased more than 13-fold when treated with 500 nM TDP43 AON. A 500 nM treatment of a STMN2 parent oligonucleotide with SEQ ID NO: 144 reduced STMN2 transcript with cryptic exon levels by 91%. A 500 nM treatment of a STMN2 oligonucleotide variant with SEQ ID NO: 1344 reduced STMN2 transcript with cryptic exon levels by 80%. A 500 nM treatment of a STMN2 oligonucleotide variant with SEQ ID NO: 1342 reduced STMN2 transcript with cryptic exon levels by 85%.

Referring to FIG. 22B, STMN2-FL was decreased by 65% when treated with 500 nM TDP43 AON. A 500 nM treatment of a STMN2 parent oligonucleotide with SEQ ID NO: 144 increased STMN-FL levels by 94% (rescued to 68%). A 500 nM treatment of a STMN2 parent oligonucleotide with SEQ ID NO: 1343 increased STMN-FL levels by 11% (rescued to 39%). A 500 nM treatment of a STMN2 parent oligonucleotide with SEQ ID NO: 1351 increased STMN-FL levels by 9% (rescued to 38%). A 500 nM treatment of a STMN2 oligonucleotide variant with SEQ ID NO: 1344 increased STMN-FL levels by 114% (rescued to 75%). A 500 nM treatment of a STMN2 parent oligonucleotide with SEQ ID NO: 1350 increased STMN-FL levels by 3% (rescued to 36%). A 500 nM treatment of a STMN2 parent oligonucleotide with SEQ ID NO: 1361 increased STMN-FL levels by 9% (rescued to 38%).

### Example 4: Neuropathy as an Indication that can be Targeted by a Stathmin-2 Cryptic Splicing Modulator

Experimentally, iCell human motor neurons (Cellular Dynamics International) were plated at 19,000 cells/well in a 96-well plate according to manufacturer's instructions. Neurons were treated with SEQ ID NO: 237 and endoporter (GeneTools, LLC.) or treated with endoporter alone in triplicate wells at day 7 post-plating. After 72 hours, SEQ ID NO: 237 STMN2 parent oligonucleotide and endoporter were washed out and MG132 added. After 18 hours, RNA was isolated, cDNA generated and multiplexed QPCR assay performed for STMN2 cryptic exon and reference GAPDH quantification.

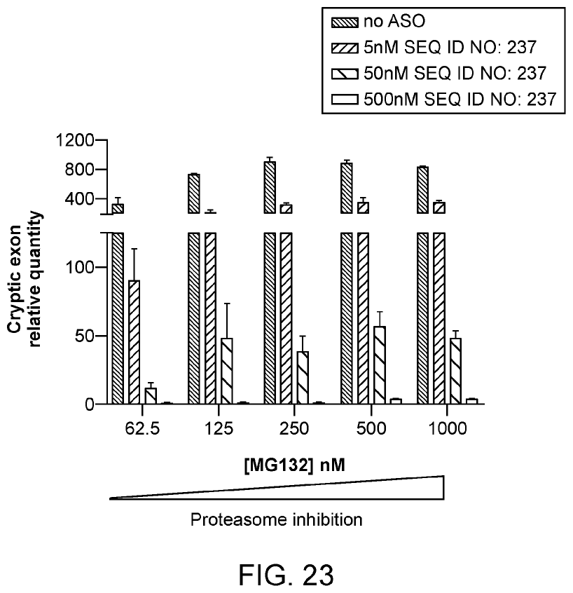

Referring to FIG. 23, it illustrates a bar graph showing reversal of cryptic exon induction using SEQ ID NO: 237 STMN2 parent oligonucleotide even in view of increasing proteasome inhibition. As a control, cells that were treated with endoporter alone (no AON) and then subsequently treated with MG132 (across all concentrations of MG132) demonstrated high levels of cryptic exon. This is indicative of TDP-43 pathology induced by proteasome inhibition in human motor neurons. Mislocalization of TDP-43 leads to STMN2 mis-splicing and increased cryptic exon expression. The addition of SEQ ID NO: 237 parent oligonucleotide reverses cryptic exon induction with high potency (IC50<5 nM). As shown in FIG. 23, increasing concentrations of SEQ ID NO: 237 (ranging from 5 nM up to 500 nM) significantly reduces the cryptic exon relative quantity.

In totality, this data establishes that the SEQ ID NO: 237 parent oligonucleotide protects against proteotoxic stress induction of cryptic exon expression. This is applicable in settings where neurons are to be protected from proteotoxic stress present in neurodegenerative disorders.

### Example 5: Dose Response Restoration of Full Length STMN2 mRNA and STMN2 Protein Using Stathmin-2 Cryptic Splicing Modulator

The experiment was performed as previously described in human neuroblastoma SY5Y cells. The cells were plated in 6-well or 96-well plates and cultured to 80% confluency. TDP-43 expression in cells were knocked down using an AON to TDP43 to express the cryptic exon, thus preventing transcription of full-length STMN2 (STMN2-FL) product. Cells were additionally co-transfected with a STMN2 oligonucleotide variant (specifically, SEQ ID NO: 1348) at varying doses (5 nM, 50 nM, 100 nM, 200 nM, and 500 nM). RNA and protein were isolated for QPCR and western blot assays.

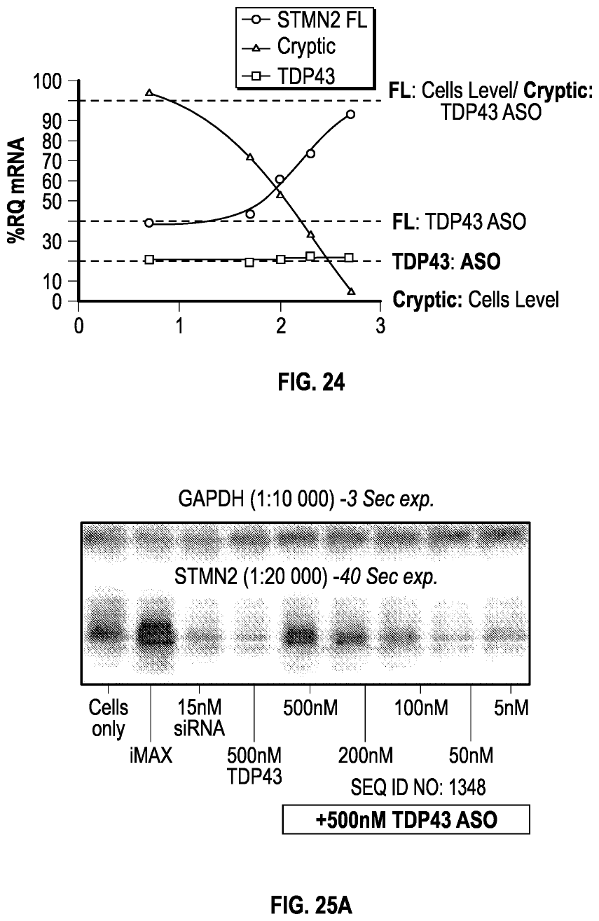

FIG. 24 shows the dose response curve illustrating increasing restoration of full length STMN2 transcript with increasing concentrations of STMN2 oligonucleotide variant with SEQ ID NO: 1348. Generally, increasing concentrations of the oligonucleotide increased full length STMN2 mRNA, decreased cryptic exon levels. Specifically, a 5 nM treatment of the STMN2 oligonucleotide variant resulted in ~40% restoration of full length STMN2 transcript. A 500 nM treatment of the STMN2 oligonucleotide variant resulted in nearly 100% restoration of full length STMN2 transcript. Additionally, the 500 nM treatment of the STMN2 oligonucleotide variant resulted in the significant reduction (close to 0%) of cryptic exon.

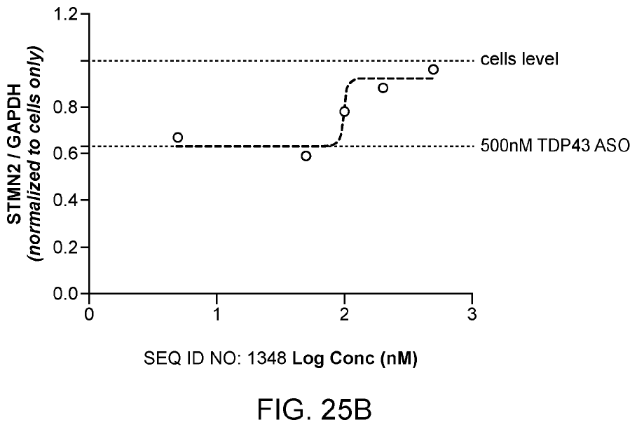

FIG. 25A shows a protein blot assay demonstrating the qualitative increase of full length STMN2 protein in response to higher concentrations of STMN2 oligonucleotide variant with SEQ ID NO: 1348. FIG. 25B shows the quantitated levels of full length STMN2 protein normalized to GAPDH in response to different concentrations of STMN2 oligonucleotide variant. Generally, both FIGS. 25A and 25B show that increasing concentrations of the STMN2 oligonucleotide variant resulted in increasing concentrations of full length STMN2 protein. Specifically, as shown in FIG. 25B, lower concentrations (5 nM and 50 nM) of the STMN2 oligonucleotide variant resulted in full length STMN2 protein concentrations that were ~60% of the control group (cell only). Notably, the 500 nM treatment of the STMN2 oligonucleotide variant resulted in nearly 100% restoration of the full length STMN2 protein (in comparison to the cell only control group).

### Example 6: STMN2 AONs with Spacer Technology Restore Full Length STMN2 and Reduces STMN2 Transcripts with a Cryptic Exon

STMN2 AONs with two or three spacers were developed. Here, a spacer is represented by Formula (I), wherein:

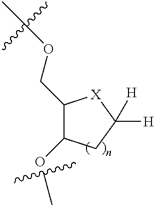

X is —O—; and
n is 1.

STMN2 AONs (e.g., STMN2 oligonucleotides each with two spacers) were tested in human motor neurons (hMN) for their ability to increase or restore full-length STMN2 mRNA (i.e., mRNA from which full-length STMN2 is translated) levels in TDP43 silenced cells. In some cases, STMN2 oligonucleotides are tested for their ability to reduce STMN2 transcripts with a cryptic exon. As described further below, the quantified percentage increase/restoration of STMN2-FL and/or percentage reduction of STMN2 transcripts with cryptic exon is described in reference to levels of STMN-FL and/or STMN2 transcripts with cryptic exon in a control group (e.g., cells treated with 500 nM TDP43 AON).

Three different STMN2 oligonucleotides with two spacers were generated. These three example STMN2 oligonucleotides are named 1) SEQ ID NO: 1589 (a 25mer with a first spacer at position 11 and a second spacer at position 22), 2) SEQ ID NO: 1590 (a 25mer with a first spacer at position 7 and a second spacer at position 14), and 3) SEQ ID NO: 1591 (a 25mer with a first spacer at position 8 and a second spacer at position 19). The STMN2 AONs are shown in Table 26.

TABLE 26
STMN2 AONs (including STMN2 parent
oligonucleotides and STMN2 oligonucleotides with
two spacers)

| Sequence | Oligonucleotide Sequence* |
|---|---|
| ID Number | (where S indicates presence |
| (SEQ ID NO) | of a Spacer) (5′→3′) |
| 144 | AATCCAATTAAGAGAGAGTGATGGG |
| 1589 | AATCCAATTASGAGAGAGTGASGGG |
| 173 | GAGTCCTGCAATATGAATATAATTT |
| 1590 | GAGTCCSGCAATASGAATATAATTT |
| 237 | GCACACATGCTCACACAGAGAGCCA |
| 1591 | GCACACASGCTCACACAGSGAGCCA |

*Unless otherwise noted, each of the nucleosides of antisense oligonucleotides shown in Table 26 are modified nucleosides with 2′-O-(2-methoxyethyl) (2′-MOE) sugar moieties, each “C” is replaced with a 5-methylcytosine (5-MeC), and all internucleoside linkages are phosphorothioate linkages. A spacer as indicated by S is not a nucleoside. S represents a spacer of Formula (Iia′) as disclosed herein.

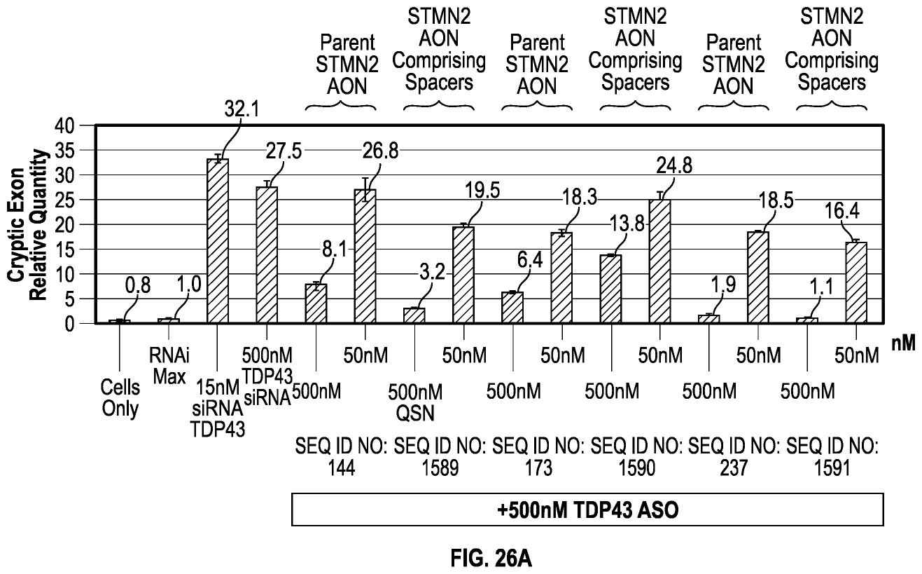

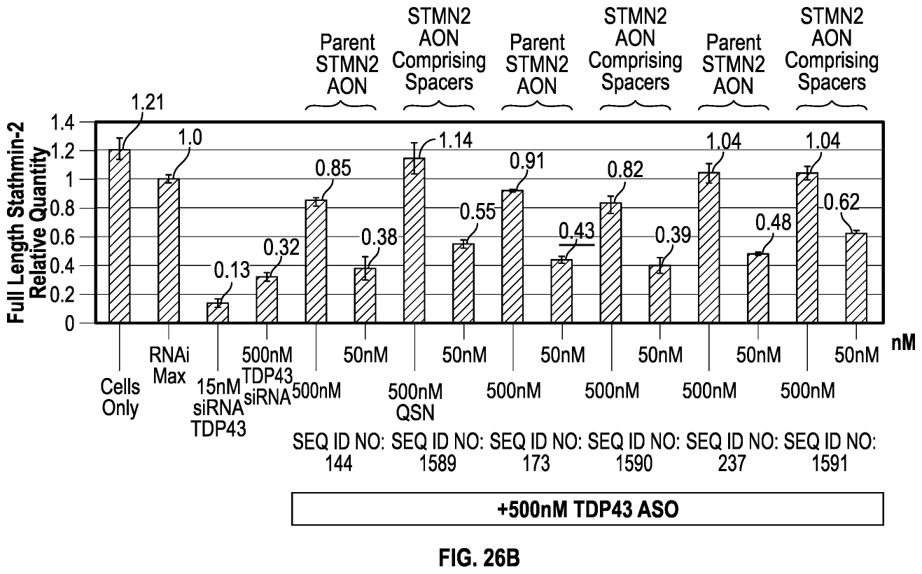

Referring to FIG. 26A, the quantity of STMN2 transcript with cryptic exon was increased more than 27-fold when treated with 500 nM TDP43 AON. A 500 nM treatment of a STMN2 parent oligonucleotide with SEQ ID NO: 144 reduced STMN2 transcript with cryptic exon levels by 71%. A 500 nM treatment of a STMN2 AON with SEQ ID NO: 1589 reduced STMN2 transcript with cryptic exon levels by 88%. Here, SEQ ID NO: 1589 exhibited further reduction of STMN2 transcripts with cryptic exon in comparison to SEQ ID NO: 144 (without two spacers.) A 500 nM treatment of a STMN2 parent oligonucleotide with SEQ ID NO: 173 reduced STMN2 transcript with cryptic exon levels by 77%. A 500 nM treatment of a STMN2 AON with SEQ ID NO: 1590 reduced STMN2 transcript with cryptic exon levels by 48%. A 500 nM treatment of a STMN2 parent oligonucleotide with SEQ ID NO: 237 reduced STMN2 transcript with cryptic exon levels by 93%. A 500 nM treatment of a STMN2 AON with SEQ ID NO: 1591 reduced STMN2 transcript with cryptic exon levels by 96%. Here, SEQ ID NO: 1591 exhibited similar reduction of STMN2 transcripts with cryptic exon in comparison to SEQ ID NO: 237 (without two spacers.)

Referring to FIG. 26B, STMN2-FL was decreased by 68% when treated with 500 nM TDP43 AON. A 500 nM treatment of a STMN2 parent oligonucleotide with SEQ ID NO: 144 increased STMN-FL levels by 165% (rescued to 85%). A 500 nM treatment of a STMN2 AON with SEQ ID NO: 1589 increased STMN-FL levels by 256% (rescued to 114%). Here, SEQ ID NO: 1589 exhibited improved restoration of STMN2 FL mRNA in comparison to SEQ ID NO: 144 (without two spacers.) A 500 nM treatment of a STMN2 parent oligonucleotide with SEQ ID NO: 173 increased STMN-FL levels by 184% (rescued to 91%). A 500 nM treatment of a STMN2 AON with SEQ ID NO: 1590 increased STMN-FL levels by 156% (rescued to 82%). Here, SEQ ID NO: 1590 exhibited similar restoration of STMN2 FL mRNA in comparison to SEQ ID NO: 173 (without two spacers.) A 500 nM treatment of a STMN2 parent oligonucleotide with SEQ ID NO: 237 increased STMN-FL levels by 225% (rescued to 104%). A 500 nM treatment of a STMN2 AON with SEQ ID NO: 1591 increased STMN-FL levels by 225% (rescued to 104%). Here, SEQ ID NO: 1591 exhibited similar restoration of STMN2 FL mRNA in comparison to SEQ ID NO: 237 (without two spacers.).

Additional example STMN2 AONs (including STMN2 AONs described above in Table 26) are shown below in Table 27. Specifically, Table 27 includes example STMN2 AONs with two spacers and STMN2 AONs with three spacers. Furthermore, Table 27 includes example STMN2 AON variants with one or more spacers that are shorter in length (e.g., 23mer, 21mer or 19mer) in comparison to STMN2 parent oligonucleotides described above in Table 26.

TABLE 27
STMN2 AONs with two or three spacers and STMN2
AON variants with two spacers.

| Sequence | Oligonucleotide Sequence* |
|---|---|
| ID Number | (where S indicates |
| (SEQ ID NO). | presence of a Spacer) (5′→3′) |
| 144 | AATCCAATTAAGAGAGAGTGATGGG |
| 1589 | AATCCAATTASGAGAGAGTGASGGG |
| 1592 | AATCCAASTAAGAGASAGTGATGSG |
| 1593 | AATCCAASTAAGAGASAGTGATSGG |
| 1594 | ASTCCAATTSAGAGAGASTGATGGG |
| 1417 | AATCCSATTASGAGAGAGSGATGGG |
| 1595 | TCCAATTSAGAGAGASTGATGGG |
| 173 | GAGTCCTGCAATATGAATATAATTT |
| 1590 | GAGTCCSGCAATASGAATATAATTT |
| 1596 | GAGTCCTSCAATATGSATATAATST |
| 1597 | GAGSCCTGCAASATGAATSTAATTT |
| 1418 | GAGTCCSGCAATASGAATATASTTT |
| 1598 | GTCCTGCSATATGAASATAAT |
| 1599 | GTCCTSCAATATGSATATAAT |
| 1419 | GTCCSGCAATASGAATATA |
| 237 | GCACACATGCTCACACAGAGAGCCA |
| 1591 | GCACACASGCTCACACAGSGAGCCA |
| 1600 | GCACACASGCTCACASAGAGAGSCA |
| 1601 | GCSCACATGSTCACACASAGAGCCA |
| 1420 | GCACACASGCTCACASAGAGSGCCA |
| 1602 | GCACACASGCTCACASAGAGAGC |
| 1603 | AATSCAATTAAGAGSGAGTGATGGG |
| 1604 | AATCCAATTASGAGAGAGTGSTGGG |
| 1605 | AATCCASTTAAGAGAGAGSGATGGG |
| 1606 | AATCCASTTAAGAGAGASTGATGGG |
| 1607 | AATCCAASTAAGSGAGASTGATGGG |
| 1608 | GAGSCCTGCAATATSAATATAATTT |
| 1609 | GAGTCCTGCASTATGAATATSATTT |
| 1610 | GAGTCCSGCAATATGAATSTAATTT |
| 1611 | GAGTCCSGCAATATGAASATAATTT |
| 1612 | GAGTCCTSCAATSTGAASATAATTT |
| 1613 | GAGTCCSGCAATSTGAATSTAATTT |
| 1614 | GAGTCSTGCAATSTGAATASAATTT |
| 1615 | GAGTSCTGCAATSTGAATATSATTT |
| 1616 | GTCCTGCSATATGSATATAAT |
| 1617 | CCTTTCTCSCGAAGGTCTTCSGCCG |
| 1618 | CTTTCTCSCGAAGGTSTTCTGCC |
| 1619 | TTTCTCTSGAAGGTCSTCTGCCG |
| 1664 | GCACACASGCSCACACAGSGAGCCA |
| 1621 | GCACACASGCTCSCACASAGAGCCA |

*Unless otherwise noted, each of the nucleosides of antisense oligonucleotides shown in Table 27 are modified nucleosides with 2′-O-(2-methoxyethyl) (2′-MOE) sugar moieties, each “C” is replaced with a 5-methylcytosine (5-MeC), and all internucleoside linkages are phosphorothioate linkages. A spacer, as indicated by S, is not a nucleoside. S represents a spacer of Formula (Iia′) as disclosed herein.

Table 13 depicts the performance of STMN2 AONs, including STMN2 AONs with two or three spacers.

STMN2 AONs that included two spacers increased levels of STMN2-FL. For example, at a dose of 200 nM ASO, SEQ ID NO: 1608 and SEQ ID NO: 1609 increased levels of STMN-FL to 0.65 and 0.78, respectively. Additionally, at a dose of 200 nM ASO, SEQ ID NO: 1610 and SEQ ID NO: 1611 increased levels of STMN-FL to 0.95 and 1.09, respectively. Notably, a number of STMN2 AONs increased levels of STMN-FL to a lesser extent. Specifically, at a 200 nM dose of STMN2 AON, SEQ ID NO: 1612, SEQ ID NO: 1613, SEQ ID NO: 1614, and SEQ ID NO: 1615 increased levels of STMN-FL to between 0.10 and 0.20.

At a dose of 200 nM AON, all STMN2 AON derived from SEQ ID NO: 197 significantly increased levels of STMN-FL. Specifically, SEQ ID NO: 1617, SEQ ID NO: 1618, and SEQ ID NO: 1619 increased levels of STMN-FL to 0.99, 0.94, and 1.00, respectively.

Altogether, these results demonstrate that different STMN2 AONs including two spacers are capable of increasing STMN-FL to levels that are close or comparable to their non-spacer counterparts (e.g., SEQ ID NO: 173 or SEQ ID NO: 197).

The differences in performance between STMN2 AONs derived from SEQ ID NO: 173, including SEQ ID NO: 1612, SEQ ID NO: 1613, SEQ ID NO: 1614, and SEQ ID NO: 1615 and STMN2 AONs derived from SEQ ID NO: 197 including SEQ ID NO: 1617, SEQ ID NO: 1618, and SEQ ID NO: 1619 may be attributable to GC content in the respective STMN2 AONs. Specifically, as shown in Table 28, STMN2 AONs derived from SEQ ID NO: 173 had below 30% GC content, which may lead to their reduced performance. In contrast, as shown in Table 28, STMN2 AONs derived from SEQ ID NO: 197 had above 40% GC content. Thus, including two or more spacers in a higher GC content AON may be preferable.

In addition to GC content, the location of spacers relative to guanine and cytosine nucleobases can also impact the performance of the STMN2 AON. For example, at a 200 nM AON dose, SEQ ID NO: 1615, SEQ ID NO: 15%, and SEQ ID NO: 1597 increased levels of STMN2-FL to 0.12, 0.26, and 0.29. Each of these STMN2 AONs have three spacers. In comparison, at a 200 nM AON dose, SEQ ID NO: 1418 increased levels of STMN2-FL to 0.73. SEQ ID NO: 1418 includes spacers that are positioned to maximize the number of spacers that are immediately preceding a guanine base. Specifically, the first and second spacers of SEQ ID NO: 1418 each respectively precede a guanine base. Thus, maximizing the number of spacers in a STMN2 AON that immediately precede a guanine base can improve the performance of the STMN2 AON.

TABLE 28
Performance of varying STMN2 AONs, including STMN2 AONs with two or three
spacers.

| | | Relative | Relative | |
|---|---|---|---|---|
| | | Quantity | Quantity | |
| | | of STMN- | of STMN- | |
| | | FL in | FL in | |
| | | response | response | |
| Sequence | Oligonucleotide Sequence* (where S | to 200 | to 50 nM | |
| ID No. | indicates presence of a Spacer) | nM ASO | ASO | GC |
| (SEQ ID NO) | (5′→3′) | Treatment | treatment | content |
| 169 | CCTGCAATATGAATATAATTTTAAA | 0.73 | 0.45 | 20% |
| 1421 | CCTGCAATATGAATATAATTTTA | 1.19 | 0.48 | 22% |
| 1422 | TGCAATATGAATATAATTTTAAA | 0.85 | 0.63 | 13% |
| 1423 | CTGCAATATGAATATAATTTTAA | 0.93 | 0.69 | 17% |
| 1424 | TGCAATATGAATATAATTTTA | 0.8 | 0.44 | 14% |
| 170 | TCCTGCAATATGAATATAATTTTAA | 1.01 | 0.46 | 20% |
| 1425 | TCCTGCAATATGAATATAATTTT | 0.83 | 0.49 | 22% |
| 1426 | CTGCAATATGAATATAATTTT | 0.83 | 0.57 | 19% |
| 171 | GTCCTGCAATATGAATATAATTTTA | 0.89 | 0.41 | 24% |
| 1346 | GTCCTGCAATATGAATATAATTT | 1.1 | 1.13 | 26% |
| 1355 | CCTGCAATATGAATATAATTT | 0.82 | 0.44 | 24% |
| 172 | AGTCCTGCAATATGAATATAATTTT | 0.79 | 0.45 | 24% |
| 1427 | AGTCCTGCAATATGAATATAATT | 0.89 | 0.52 | 26% |
| 1428 | TCCTGCAATATGAATATAATT | 1.18 | 0.66 | 24% |
| 252 | CTCTCTCGCACACACGCACACATGC | 0.67 | 0.43 | 60% |
| 1432 | CTCTCGCACACACGCACACATGC | 0.67 | 0.52 | 61% |
| 1433 | CTCTCTCGCACACACGCACACAT | 0.63 | 0.24 | 57% |
| 1434 | TCTCTCGCACACACGCACACATG | 0.73 | 0.45 | 57% |
| 1435 | CTCTCGCACACACGCACACAT | 0.84 | 0.36 | 57% |
| 173 | GAGTCCTGCAATATGAATATAATTT | 1.12 | 0.6 | 28% |
| 1608 | GAGSCCTGCAATATSAATATAATTT | 0.65 | 0.19 | 24% |
| 1609 | GAGTCCTGCASTATGAATATSATTT | 0.78 | 0.25 | 28% |
| 1610 | GAGTCCSGCAATATGAATSTAATTT | 0.95 | 0.43 | 28% |
| 1611 | GAGTCCSGCAATATGAASATAATTT | 1.09 | 0.32 | 28% |
| 1612 | GAGTCCTSCAATSTGAASATAATTT | 0.15 | 0.08 | 24% |
| 1613 | GAGTCCSGCAATSTGAATSTAATTT | 0.2 | 0.13 | 28% |
| 1614 | GAGTCSTGCAATSTGAATASAATTT | 0.13 | 0.18 | 24% |
| 1615 | GAGTSCTGCAATSTGAATATSATTT | 0.12 | 0.12 | 24% |
| 1596 | GAGTCCTSCAATATGSATATAATST | 0.26 | 0.13 | 24% |
| 1597 | GAGSCCTGCAASATGAATSTAATTT | 0.29 | 0.17 | 28% |
| 1418 | GAGTCCSGCAATASGAATATASTTT | 0.73 | 0.24 | 28% |
| 1598 | GTCCTGCSATATGAASATAAT | 0.72 | 0.31 | 29% |
| 1599 | GTCCTSCAATATGSATATAAT | 0.1 | 0.16 | 24% |
| 1616 | GTCCTGCSATATGSATATAAT | 0.77 | 0.23 | 29% |
| 197 | CCTTTCTCTCGAAGGTCTTCTGCCG | 1.04 | 0.44 | 56% |
| 1429 | TTTCTCTCGAAGGTCTTCTGCCG | 1.35 | 1.06 | 48% |
| 1430 | CCTTTCTCTCGAAGGTCTTCTGC | 0.98 | 0.44 | 48% |
| 1431 | CTTTCTCTCGAAGGTCTTCTGCC | 1.33 | 0.55 | 48% |
| 1617 | CCTTTCTCSCGAAGGTCTTCSGCCG | 0.99 | 0.69 | 56% |
| 1618 | CTTTCTCSCGAAGGTSTTCTGCC | 0.94 | 0.58 | 48% |
| 1619 | TTTCTCTSGAAGGTCSTCTGCCG | 1 | 0.54 | 48% |

*Unless otherwise noted, each of the nucleosides of antisense oligonucleotides shown in Table 28 are modified nucleosides with 2′-O-(2-methoxyethyl) (2′-MOE) sugar moieties, each “C” is replaced with a 5-methylcytosine (5-MeC), and all internucleoside linkages are phosphorothioate linkages. A spacer as indicated by S is not a nucleoside. S represents a spacer of Formula (Iia′) as disclosed herein.

### Example 7: Additional Experiments Demonstrate STMN2 AONs with Spacer Technology Restore Ful Length STMN2 and Reduces STMN2 Transcripts with a Cryptic Exon

STMN2 AONs with one, two, or three spacers were developed. Generally, in this Example, except for SEQ ID NO: 1649 described below, a spacer is represented by Formula (I), wherein:

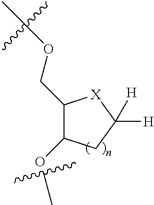

X is —O—; and
n is 1.

For SEQ ID NO: 1649, each spacer included in the ASO is represented by Formula (I), wherein:

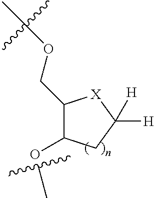

X is —O—; and
n is 2.

STMN2 AONs with spacers were characterized and compared to STMN2 AON without spacer counterparts. Specifically, the melting temperature of STMN2 AON with and without spacers were determined to demonstrate the structural differences of the STMN2 AONs. Table 29 shows the different melting temperatures of STMN2 AONs across two different replicates. STMN2 AONs with two spacers exhibited a lower melting temperature (approximately 10° C. lower) in comparison to STMN2 AONs without spacers.

TABLE 29
Melting temperatures of STMN2 AONs with and without spacers.

| ASO + RNA | Tm (° C.) | Tm (° C.) | ΔTm ° C. | ΔTm ° C. | |
|---|---|---|---|---|---|
| target (25bases) | Replicate 1 | Replicate 2 | Replicate 1 | Replicate 2 | % GC |
| SEQ ID NO: 237 | 86.6 | 86.5 | 11.6 | 11.4 | 56 |
| (no spacer) | | | | | |
| SEQ ID NO: 1591 | 75.0 | 75.1 | | | |
| (2 spacers) | | | | | |
| SEQ ID NO: 144 | 75.5 | 75.5 | 9.5 | 9.7 | 40 |
| (no spacer) | | | | | |
| SEQ ID NO: 1589 | 66.0 | 65.8 | | | |
| (2 spacers) | | | | | |
| SEQ ID NO: 173 | 71.2 | 71.1 | 13.5 | 13.5 | 28 |
| (no spacer) | | | | | |
| SEQ ID NO: 1590 | 57.7 | 57.6 | | | |
| (2 spacers) | | | | | |

* Unless otherwise noted, each of the nucleosides of antisense oligonucleotides identified in Table 29 are modified nucleosides with 2′-O-(2-methoxyethyl) (2′-MOE) sugar moieties, each “C” is replaced with a 5-methylcytosine (5-MeC), and all internucleoside linkages are phosphorothioate linkages. A spacer is not a nucleoside and is represented by Formula (Iia′) as disclosed herein.

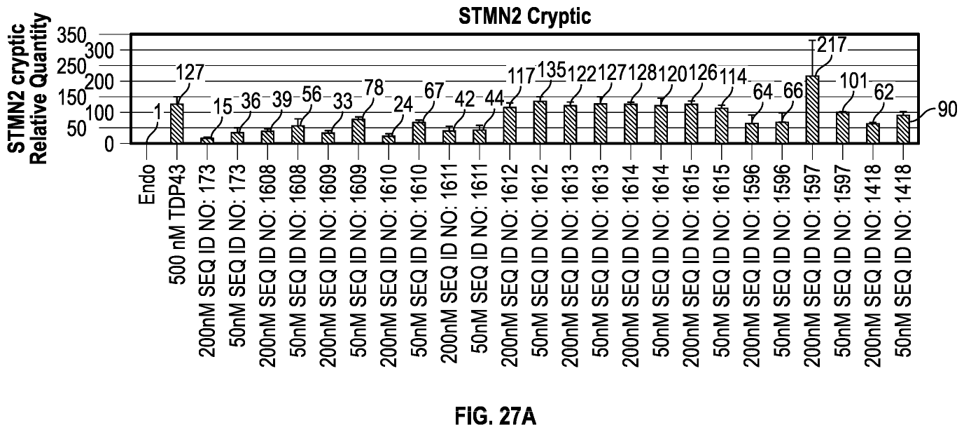

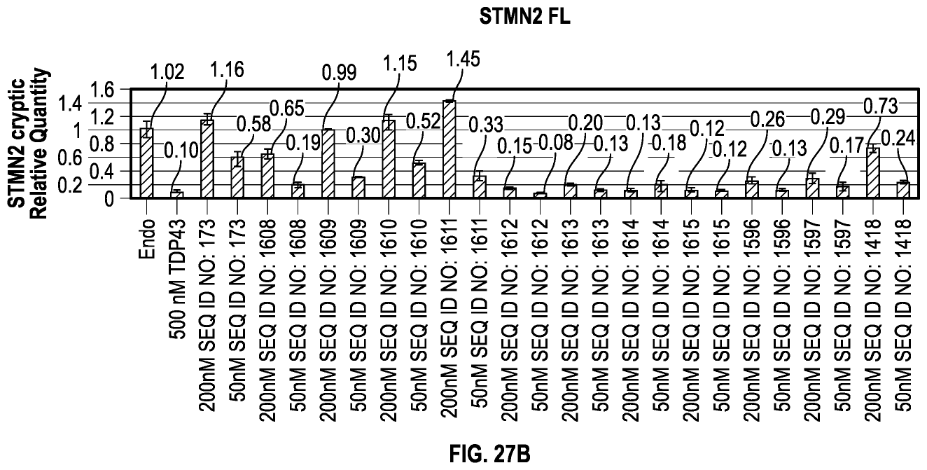

STMN2 AONs (e.g., STMN2 oligonucleotides with one, two, or three spacers) were tested for their ability to increase or restore full-length STMN2 mRNA (i.e., mRNA from which full-length STMN2 is translated) levels in TDP43 silenced cells. In some cases, STMN2 oligonucleotides are tested for their ability to reduce STMN2 transcripts with a cryptic exon. FIGS. 27-35 show effects of STMN2 AONs with spacers in increasing full-length STMN2 mRNA (“STMN2 FL”) and/or in reducing STMN2 transcripts with a cryptic exon (“STMN2 cryptic”). Furthermore, Table 30 identifies the respective STMN2 AONs as well as their respective performances. Treatment groups are identified on the X-axis of FIGS. 27-35 and include the concentration of specific AON sequences. Here, specific AON sequences are labeled according to their corresponding SEQ ID NO.

FIG. 27A is a bar graph showing the results of RT-qPCR analysis of STMN2 transcript with cryptic exon mRNA levels in the presence of TDP43 antisense, and reduction of the STMN2 transcript with cryptic exon miRNA levels across different dosages of STMN2 AONs including SEQ ID NO: 173, SEQ ID NO: 1608, SEQ ID NO: 1609, SEQ ID NO: 1610, SEQ ID NO: 1611, SEQ ID NO: 1612, SEQ ID NO: 1613, SEQ ID NO: 1614, SEQ ID NO: 1615, SEQ ID NO: 1596, SEQ ID NO: 1597, and SEQ ID NO: 1418. FIG. 27B is a bar graph showing the results of RT-qPCR analysis of STMN2 full-length mRNA levels in the presence of TDP43 antisense, and restoration of the full-length STMN2 transcript across different dosages of STMN2 AONs including SEQ ID NO: 173, SEQ ID NO: 1608, SEQ ID NO: 1609, SEQ ID NO: 1610, SEQ ID NO: 1611, SEQ ID NO: 1612, SEQ ID NO: 1613, SEQ ID NO: 1614, SEQ ID NO: 1615, SEQ ID NO: 1596, SEQ ID NO: 1597, and SEQ ID NO: 1418. Generally, FIGS. 27A and 27B demonstrate the performance of STMN2 AONs with spacers (e.g., SEQ ID NO: 1608, SEQ ID NO: 1609, SEQ ID NO: 1610, SEQ ID NO: 1611, SEQ ID NO: 1612, SEQ ID NO: 1613, SEQ ID NO: 1614, SEQ ID NO: 1615, SEQ ID NO: 15%, SEQ ID NO: 1597, and SEQ ID NO: 1418) in comparison to STMN2 AON without spacers (SEQ ID NO: 173). Here, a number of STMN2 AON with spacers perform as well, or outperform the STMN2 AON without spacers (SEQ ID NO: 173). Specifically, 200 nM of SEQ ID NO: 1609, SEQ ID NO: 1610, and SEQ ID NO: 1611 achieve comparable levels of STMN2 transcript with cryptic exon mRNA levels and STMN2 full-length mRNA levels in the presence of TDP43 in comparison to STMN2 AON without spacers (SEQ ID NO: 173).

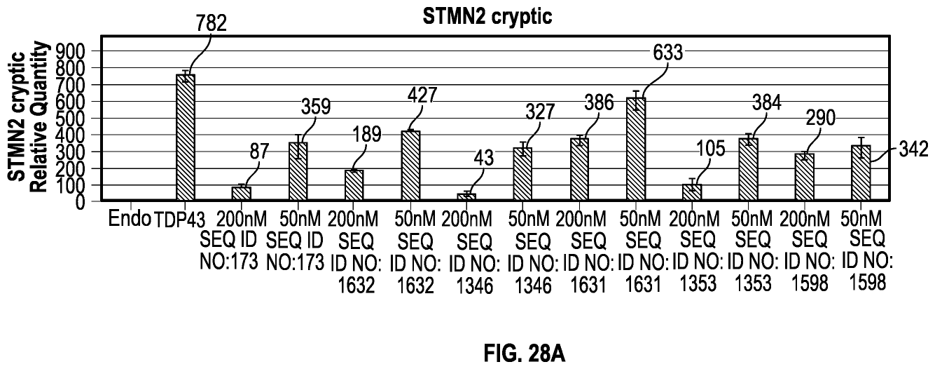

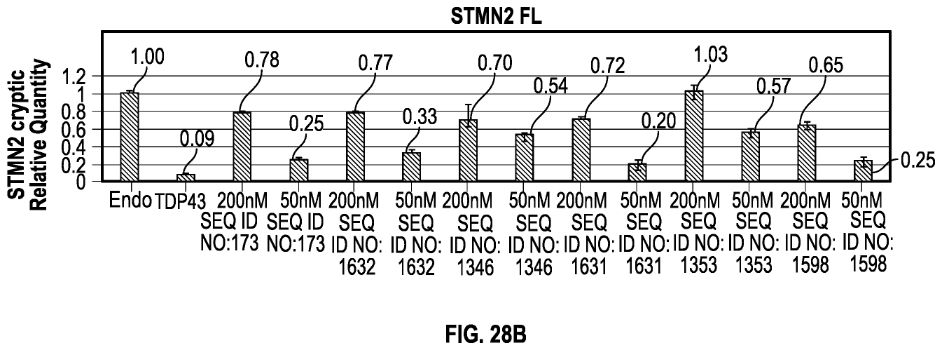

FIG. 28A is a bar graph showing the results of RT-qPCR analysis of STMN2 transcript with cryptic exon mRNA levels in the presence of TDP43 antisense, and reduction of the STMN2 transcript with cryptic exon mRNA levels across different dosages of STMN2 AONs including SEQ ID NO: 173, SEQ ID NO: 1632, SEQ ID NO: 1346, SEQ ID NO: 1631, SEQ ID NO: 1353, and SEQ ID NO: 1598. FIG. 28B is a bar graph showing the results of RT-qPCR analysis of STMN2 full-length mRNA levels in the presence of TDP43 antisense, and restoration of the full-length STMN2 transcript across different dosages of STMN2 AONs including SEQ ID NO: 173, SEQ ID NO: 1632, SEQ ID NO: 1346, SEQ ID NO: 1631, SEQ ID NO: 1353, and SEQ ID NO: 1598. Generally, FIGS. 28A and 28B demonstrate the performance of STMN2 AONs with spacers (e.g., SEQ ID NO: 1632, SEQ ID NO: 1631, and SEQ ID NO: 1598) in comparison to their STMN2 AON counterparts without spacer (e.g., SEQ ID NO: 173, SEQ ID NO: 1346, and SEQ ID NO: 1353). Here, a 50 nM or 200 nM dose of SEQ ID NO: 1632 achieves comparable levels of STMN2 transcript with cryptic exon mRNA levels and STMN2 full-length mRNA levels in the presence of TDP43 in comparison to a 50 nM or 200 nM dose of the STMN2 AON counterpart without spacers (SEQ ID NO: 173). A 200 nM dose of SEQ ID NO: 1631 achieves comparable levels of STMN2 full-length mRNA levels in the presence of TDP43 in comparison to 200 nM dose of the STMN2 AON counterpart without spacers (SEQ ID NO: 1346).

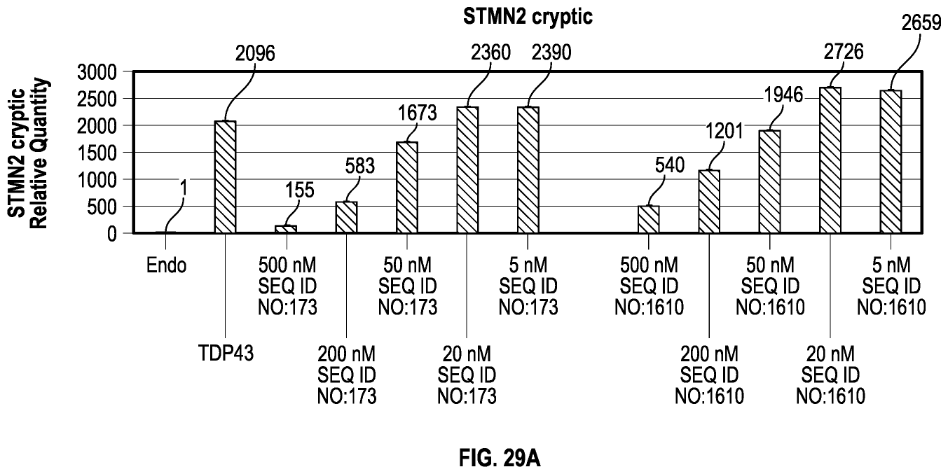

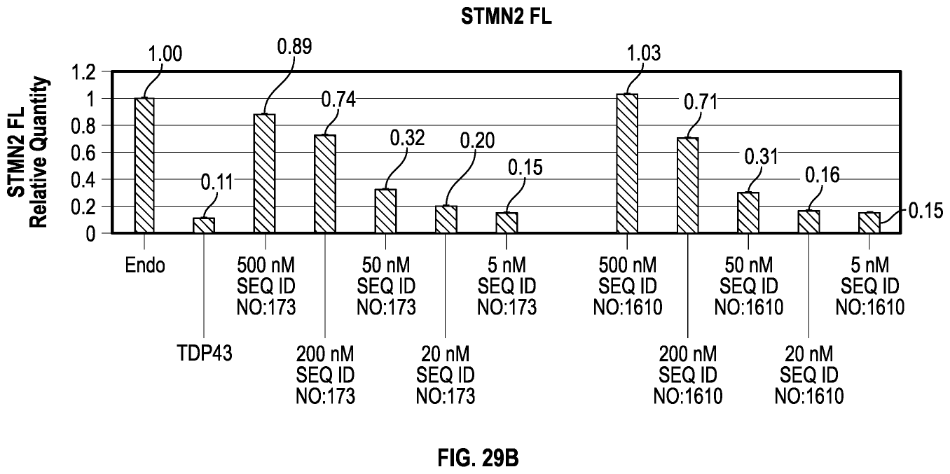

FIG. 29A is a bar graph showing the results of RT-qPCR analysis of STMN2 transcript with cryptic exon mRNA levels in the presence of TDP43 antisense, and reduction of the STMN2 transcript with cryptic exon mRNA levels across different dosages of STMN2 AONs including SEQ ID NO: 173 and SEQ ID NO: 1610. FIG. 29B is a bar graph showing the results of RT-qPCR analysis of STMN2 full-length mRNA levels in the presence of TDP43 antisense, and restoration of the full-length STMN2 transcript across different dosages of STMN2 AONs including SEQ ID NO: 173 and SEQ ID NO: 1610. Generally, FIGS. 29A and 29B demonstrate the performance of STMN2 AONs with spacers (e.g., SEQ ID NO: 1610) in comparison to the STMN2 AON counterpart without spacers (e.g., SEQ ID NO: 173). Across the different doses (e.g., 500 nM, 200 nM, 50 nM, 20 nM, and 5 nM), SEQ ID NO: 1610 achieves comparable levels of STMN2 transcript with cryptic exon mRNA levels and STMN2 full-length mRNA levels in the presence of TDP43 in comparison to the STMN2 AON counterpart without spacers (SEQ ID NO: 173).

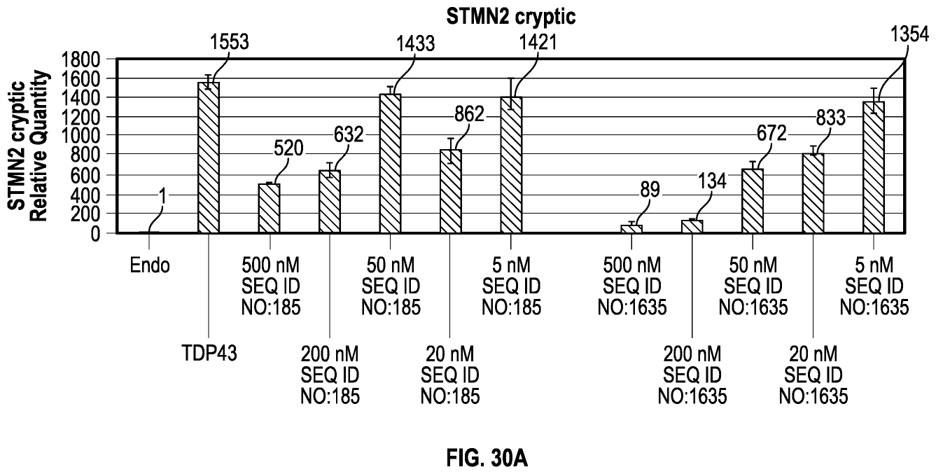

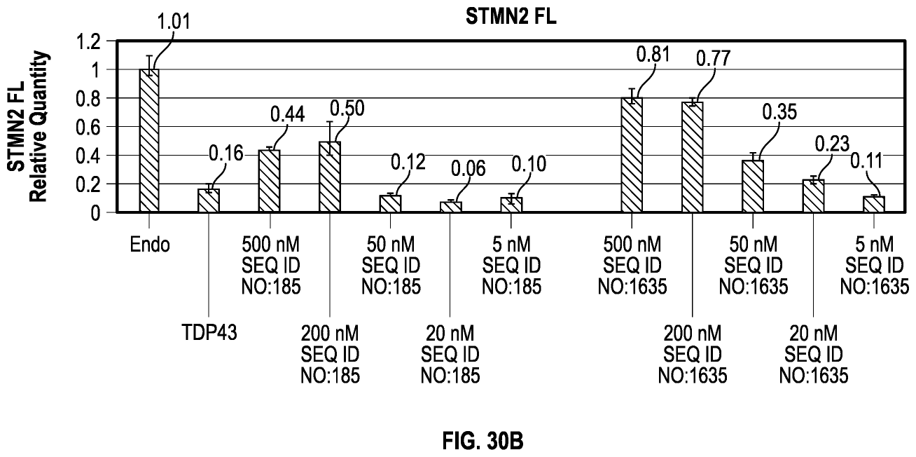

FIG. 30A is a bar graph showing the results of RT-qPCR analysis of STMN2 transcript with cryptic exon mRNA levels in the presence of TDP43 antisense, and reduction of the STMN2 transcript with cryptic exon mRNA levels across different dosages of STMN2 AONs including SEQ ID NO: 185 and SEQ ID NO: 1635. FIG. 30B is a bar graph showing the results of RT-qPCR analysis of STMN2 full-length mRNA levels in the presence of TDP43 antisense, and restoration of the full-length STMN2 transcript across different dosages of STMN2 AONs including SEQ ID NO: 185 and SEQ ID NO: 1635. Generally, FIGS. 30A and 30B demonstrate the performance of STMN2 AONs with spacers (e.g., SEQ ID NO: 1635) in comparison to the STMN2 AON counterpart without spacers (e.g., SEQ ID NO: 185). Across the different doses (e.g., 500 nM, 200 nM, 50 nM, 20 nM, and 5 nM), SEQ ID NO: 1610 achieves comparable or reduced levels of STMN2 transcript with cryptic exon mRNA levels and comparable or increased STMN2 full-length mRNA levels in the presence of TDP43 in comparison to the STMN2 AON counterpart without spacers (SEQ ID NO: 185).

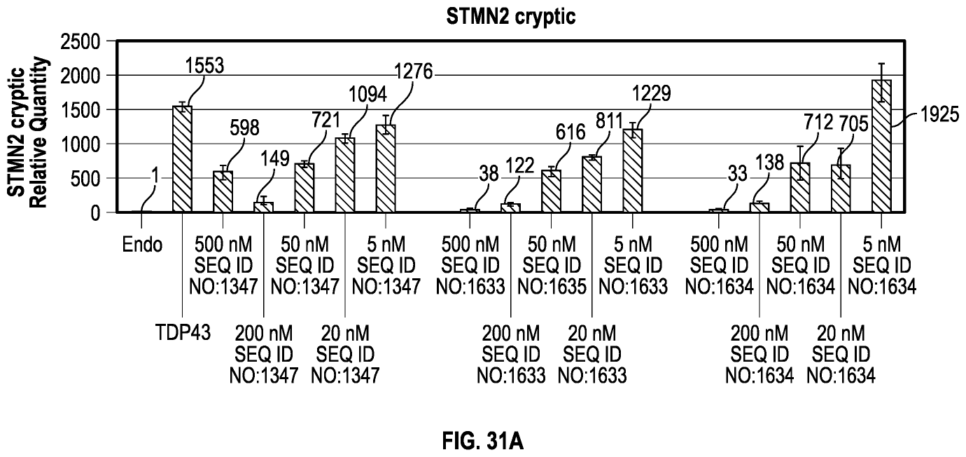

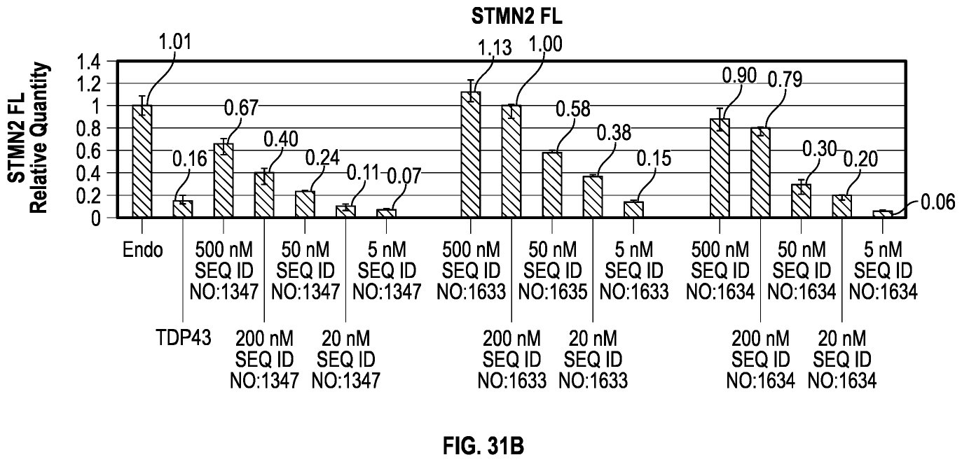

FIG. 31A is a bar graph showing the results of RT-qPCR analysis of STMN2 transcript with cryptic exon mRNA levels in the presence of TDP43 antisense, and reduction of the STMN2 transcript with cryptic exon mRNA levels across different dosages of STMN2 AONs including SEQ ID NO: 1347, SEQ ID NO: 1633, and SEQ ID NO: 1634. FIG. 31B is a bar graph showing the results of RT-qPCR analysis of STMN2 full-length mRNA levels in the presence of TDP43 antisense, and restoration of the full-length STMN2 transcript across different dosages of STMN2 AONs including SEQ ID NO: 1347, SEQ ID NO: 1633, and SEQ ID NO: 1634. Generally, FIGS. 31A and 31B demonstrate the performance of STMN2 AONs with spacers (e.g., SEQ ID NO: 1633 and SEQ ID NO: 1634) in comparison to the STMN2 AON counterpart without spacers (e.g., SEQ ID NO: 1347). Across the different doses (e.g., 500 nM, 200 nM, 50 nM, 20 nM, and 5 nM), SEQ ID NO: 1633 achieves comparable or reduced levels of STMN2 transcript with cryptic exon mRNA levels and comparable or increased STMN2 full-length mRNA levels in the presence of TDP43 in comparison to the STMN2 AON counterpart without spacers (SEQ ID NO: 1347). Similarly, across the different doses (e.g., 500 nM, 200 nM, 50 nM, 20 nM, and 5 nM), SEQ ID NO: 1634 achieves comparable or reduced levels of STMN2 transcript with cryptic exon mRNA levels and comparable or increased STMN2 full-length mRNA levels in the presence of TDP43 in comparison to the STMN2 AON counterpart without spacers (SEQ ID NO: 1347).

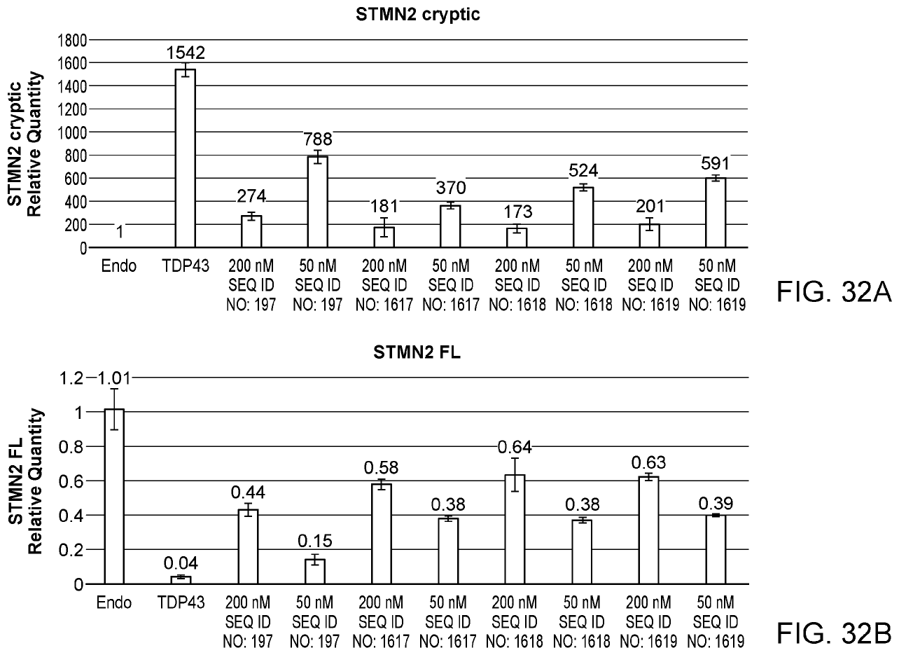

FIG. 32A is a bar graph showing the results of RT-qPCR analysis of STMN2 transcript with cryptic exon mRNA levels in the presence of TDP43 antisense, and reduction of the STMN2 transcript with cryptic exon mRNA levels across different dosages of STMN2 AONs including SEQ ID NO: 197, SEQ ID NO: 1617, SEQ ID NO: 1618, and SEQ ID NO: 1619. FIG. 32B is a bar graph showing the results of RT-qPCR analysis of STMN2 full-length mRNA levels in the presence of TDP43 antisense, and restoration of the full-length STMN2 transcript across different dosages of STMN2 AONs including SEQ ID NO: 197, SEQ ID NO: 1617, SEQ ID NO: 1618, and SEQ ID NO: 1619. Generally, FIGS. 32A and 32B demonstrate the performance of STMN2 AONs with spacers (e.g., SEQ ID NO: 1617, SEQ ID NO: 1618, and SEQ ID NO: 1619) in comparison to the STMN2 AON counterpart without spacers (e.g., SEQ ID NO: 197). At a 50 nM or 200 nM dose, SEQ ID NO: 1617 achieves comparable or reduced levels of STMN2 transcript with cryptic exon mRNA levels and comparable or increased STMN2 full-length mRNA levels in the presence of TDP43 in comparison to a 50 nM or 200 nM dose of the STMN2 AON counterpart without spacers (SEQ ID NO: 197). At a 50 nM or 200 nM dose, SEQ ID NO: 1618 achieves comparable or reduced levels of STMN2 transcript with cryptic exon mRNA levels and comparable or increased STMN2 full-length mRNA levels in the presence of TDP43 in comparison to a 50 nM or 200 nM dose of the STMN2 AON counterpart without spacers (SEQ ID NO: 197). At a 50 nM or 200 nM dose, SEQ ID NO: 1619 achieves comparable or reduced levels of STMN2 transcript with cryptic exon mRNA levels and comparable or increased STMN2 full-length mRNA levels in the presence of TDP43 in comparison to a 50 nM or 200 nM dose of the STMN2 AON counterpart without spacers (SEQ ID NO: 197).

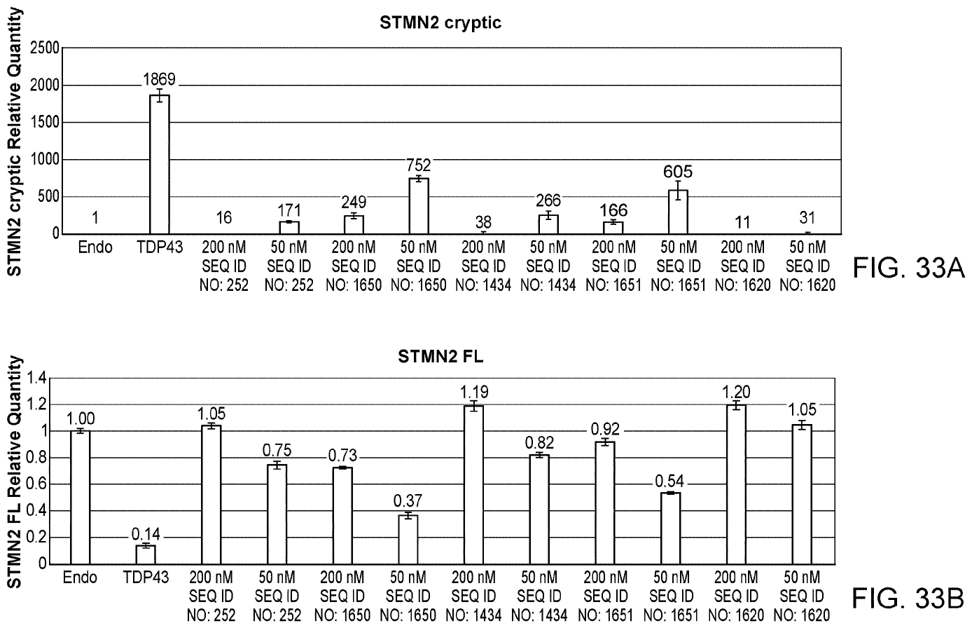

FIG. 33A is a bar graph showing the results of RT-qPCR analysis of STMN2 transcript with cryptic exon mRNA levels in the presence of TDP43 antisense, and reduction of the STMN2 transcript with cryptic exon mRNA levels across different dosages of STMN2 AONs including SEQ ID NO: 252, SEQ ID NO: 1650, SEQ ID NO: 1434, SEQ ID NO: 1651, and SEQ ID NO: 1620. FIG. 33B is a bar graph showing the results of RT-qPCR analysis of STMN2 full-length mRNA levels in the presence of TDP43 antisense, and restoration of the full-length STMN2 transcript across different dosages of STMN2 AONs including SEQ ID NO: 252, SEQ ID NO: 1650, SEQ ID NO: 1434, SEQ ID NO: 1651, and SEQ ID NO: 1620. Generally, FIGS. 33A and 33B demonstrate the performance of STMN2 AONs with spacers (e.g., SEQ ID NO: 1620) in comparison to the STMN2 AON counterpart without spacers (e.g., SEQ ID NO: 252, SEQ ID NO: 1650, SEQ ID NO: 1434, and SEQ ID NO: 1651). At a 50 nM or 200 nM dose, SEQ ID NO: 1620 achieves comparable or reduced levels of STMN2 transcript with cryptic exon mRNA levels and comparable or increased STMN2 full-length mRNA levels in the presence of TDP43 in comparison to a 50 nM or 200 nM dose of the STMN2 AON counterparts without spacers (SEQ ID NO: 252, SEQ ID NO: 1650, SEQ ID NO: 1434, and SEQ ID NO: 1651).

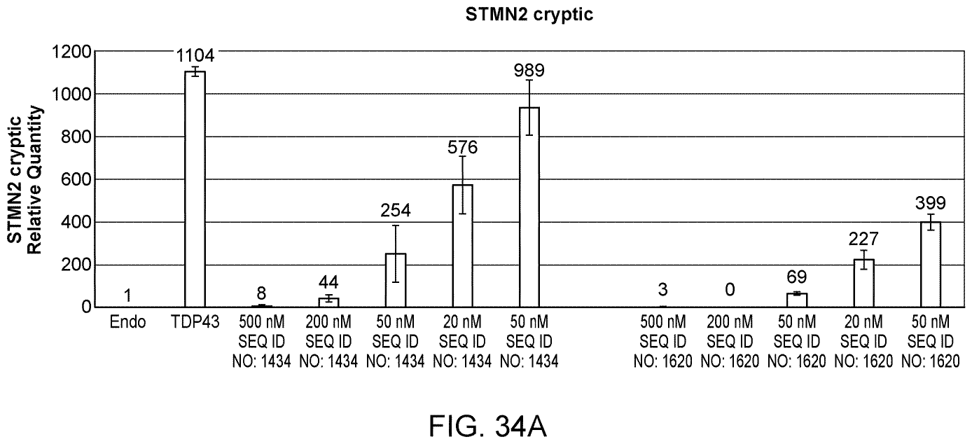

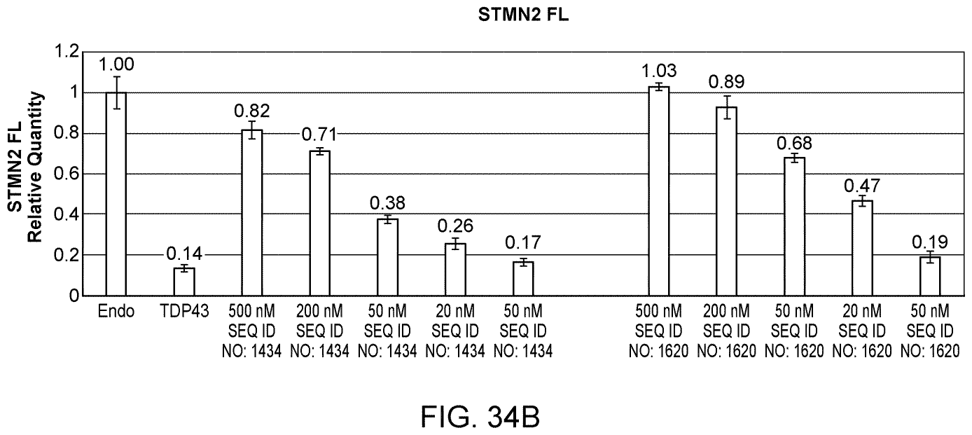

FIG. 34A is a bar graph showing the results of RT-qPCR analysis of STMN2 transcript with cryptic exon mRNA levels in the presence of TDP43 antisense, and reduction of the STMN2 transcript with cryptic exon mRNA levels across different dosages of STMN2 AONs including SEQ ID NO: 1434 and SEQ ID NO: 1620. FIG. 34B is a bar graph showing the results of RT-qPCR analysis of STMN2 full-length mRNA levels in the presence of TDP43 antisense, and restoration of the full-length STMN2 transcript across different dosages of STMN2 AONs including SEQ ID NO: 1434 and SEQ ID NO: 1620. Generally, FIGS. 34A and 34B demonstrate the performance of STMN2 AONs with spacers (e.g., SEQ ID NO: 1620) in comparison to the STMN2 AON counterpart without spacers (e.g., SEQ ID NO: 1434). Across the different doses (e.g., 500 nM, 200 nM, 50 nM, 20 nM, and 5 nM), SEQ ID NO: 1620 achieves reduced levels of STMN2 transcript with cryptic exon mRNA levels and increased STMN2 full-length mRNA levels in the presence of TDP43 in comparison to the STMN2 AON counterpart without spacers (SEQ ID NO: 1434).

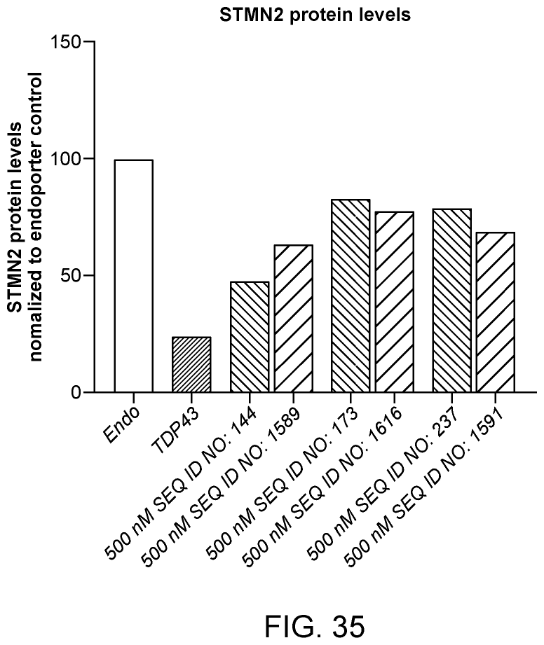

FIG. 35 is a bar graph showing normalized STMN2 protein levels following treatment with TDP43 antisense and restoration using 500 nM STMN2 AONs including SEQ ID NO: 144, SEQ ID NO: 1589, SEQ ID NO: 173, SEQ ID NO: 1616, SEQ ID NO: 237, and SEQ ID NO: 1591. Generally, FIG. 35 demonstrates the performance of STMN2 AONs with spacers (e.g., SEQ ID NO: 1589, SEQ ID NO: 1616, and SEQ ID NO: 1591) in comparison to their STMN2 AON counterparts without spacers (e.g., SEQ ID NO: 144, SEQ ID NO: 173, SEQ ID NO: 237). Generally, STMN2 AONs with spacers are able to achieve comparable levels of STMN2 protein levels in comparison to their STMN2 AON counterparts. Specifically, SEQ ID NO: 1589 achieves comparable levels of STMN2 protein levels in comparison to SEQ ID NO: 144. SEQ ID NO: 1616 achieves comparable levels of STMN2 protein levels in comparison to SEQ ID NO: 173. SEQ ID NO: 1591 achieves comparable levels of STMN2 protein levels in comparison to SEQ ID NO: 237.

Referring to Tables 30 and 31A-31C, they show the performance of STMN2 AONs with spacers (e.g., Table 30 and Tables 31B-31C) and performance of STMN2 AONs without spacers (e.g., Table 31A) in human motor neurons. RT-qPCR results for STMN2 full-length transcript provided in Tables 30 and 31A-31C are normalized values using the equation ((RQASO−RQTDP43)/(Rqendo−RQTDP43))*100 where RQ refers to Relative Quantity described above. RT-qPCR results for STMN2 transcript with a cryptic exon provided in Tables 30 and 31A-31C are normalized values using the equation (1−((RQASO−RQTDP43)/(RQendo−RQTDP43)))*100 where RQ refers to Relative Quantity described above. Each RT-qPCR experiment was run in triplicate wells and performed N number of independent replicate runs. Standard deviation or SD is calculated as the SD between each run. Where N=1, SD was reported as the standard deviation between the triplicate well results in the single experiment. Notably, as shown in Table 30, a 200 nM dose of SEQ ID NO: 1631 (GTCCTGCSATATGAASATAATT with two spacers) rescued full length STMN2 mRNA to 69% and reduced STMN2 transcript with cryptic exon levels to 49% (reduced by 51%).

Additionally, as shown in Table 30, a 200 nM dose of SEQ ID NO: 1633 (GTCTTCTSCCGAGTCSTGCAATA with two spacers) rescued full length STMN2 mRNA to 83% and reduced STMN2 transcript with cryptic exon levels to 10% (reduced by 90%). Comparatively, as shown in Table 31A, a 200 nM dose of SEQ ID NO: 1347 (GTCTTCTGCCGAGTCCTGCAATA with no spacers) rescued full length STMN2 mRNA to 40.2% and reduced STMN2 transcript with cryptic exon levels to 20.8% (reduced by 80.2%). This indicates that the addition of spacers improves the performance of SEQ ID NO: 1633 in comparison to the STMN2 AON counterpart without spacers (e.g., SEQ ID NO: 1347).

Additionally, as shown in Table 30, a 200 nM dose of SEQ ID NO: 1618 (CTTCTCSCGAAGGTSTTCTGCC with two spacers) rescued full length STMN2 mRNA to 82% and reduced STMN2 transcript with cryptic exon levels to 11% (reduced by 89%). A 200 nM dose of SEQ ID NO: 1619 (TTTCTCTSGAAGGTCSTCTGCCG with two spacers) rescued full length STMN2 mRNA to 80% and reduced STMN2 transcript with cryptic exon levels to 12% (reduced by 88%). Comparatively, as shown in Table 31A, a 200 nM dose of SEQ ID NO: 197 (CCTTTCTCTCGAAGGTCTTCTGCCG with no spacers) rescued full length STMN2 mRNA to 79.3% and reduced STMN2 transcript with cryptic exon levels to 12.1% (reduced by 87.9%). Here, at 200 nM dose, the performance of STMN2 AONs with two spacers (e.g., SEQ ID NO: 1618 and SEQ ID NO: 1619) is comparable to the STMN2 AON counterpart without spacers (e.g., SEQ ID NO: 197). Notably, at a 50 nM dose, the performance of STMN2 AONs with two spacers (e.g., SEQ ID NO: 1618 and SEQ ID NO: 1619) is improve din comparison to the STMN2 AON counterpart without spacers (e.g., SEQ ID NO: 197). Specifically, at the 50 nM dose, SEQ ID NO: 1618 rescued full length STMN2 mRNA to 46% and SEQ ID NO: 1619 rescued full length STMN2 mRNA to 42% whereas SEQ ID NO: 197 (without spacers) rescued full length STMN2 mRNA to 26.7%.

Additionally, as shown in Table 30, a 200 nM dose of SEQ ID NO: 1620 (TCTCTCGSACACACGSACACATG with two spacers) rescued full length STMN2 mRNA to 103% and reduced STMN2 transcript with cryptic exon levels to 1% (reduced by 99%). A 50 nM dose of SEQ ID NO: 1620 rescued full length STMN2 mRNA to 74% and reduced STMN2 transcript with cryptic exon levels to 5% (reduced by 95%). Comparatively, as shown in Table 31A, a 200 nM dose and 50 nM dose of SEQ ID NO: 1434 (TCTCTCGCACACACGCACACATG with no spacers) rescued full length STMN2 mRNA to 77.5% and 16.6%, respectively and reduced STMN2 transcript with cryptic exon levels to 2.7% (reduced by 97.3%) and 18.3% (reduced by 81.7%), respectively. This indicates that the addition of spacers improves the performance of SEQ ID NO: 1620 in comparison to the STMN2 AON counterpart without spacers (e.g., SEQ ID NO: 1434).

Additional STMN2 AONs were developed with LNA modifications and evaluated for their ability to restore STMN2 FL while reducing STMN2 cryptic levels. Tables 31D and 31E below shows the results of the evaluation.

TABLE 30
Performance of STMN2 AONs (STMN2 oligonucleotides with one, two, or three spacers).

| | | QPCR potency in | | QPCR potency in | |
|---|---|---|---|---|---|
| | Oligonucleotide Sequence^{#} (where S | hMN STMN2 FL | | hMN STMN2 cryptic | |
| SEQ ID | indicates presence of a Spacer) | 50 nM | 200 nM | 50 nM | 200 nM |
| NO: | (5′→3′) | Mean | Mean | Mean | Mean |
| 1622 | TGCAATASGAATATASTTTTAAA | 1 | 47 | 76 | 340 |
| 1623 | TCCTGCASTATGAATSTAATTTT | 6 | 31 | 82 | 100 |
| 1624 | CTGCAATATGSATATAATTTT | 5 | 11 | 104 | 61 |
| 1625 | CTGCAATSTGAATATSATTTTAA | 2 | −4 | 116 | 147 |
| 1626 | CCTGCAATATSAATATAATTT | 2 | 42 | 65 | 59 |
| 1627 | TCCTGCAATASGAATATAATT | 19 | 65 | 85 | 36 |
| 1628 | GTCCTGCSATATGAASATAAT | 20 | 65 | 79 | 45 |
| 1629 | GTCCTSCAATATGSATATAAT | 5 | 13 | 119 | 133 |
| 1630 | GTCCTGCSATATGSATATAAT | 16 | 71 | 97 | 51 |
| 1631 | GTCCTGCSATATGAASATAATTT | 13 | 69 | 81 | 49 |
| 1596 | GAGTCCTSCAATATGSATATAATST | 3 | 18 | 52 | 50 |
| 1597 | GAGSCCTGCAASATGAATSTAATTT | 7 | 20 | 79 | 82 |
| 1418 | GAGTCCSGCAATASGAATATASTTT | 15 | 69 | 70 | 48 |
| 1632 | GAGTCCTGCAATATSAATATAATTT | 27 | 75 | 55 | 24 |
| 1608 | GAGSCCTGCAATATSAATATAATTT | 10 | 60 | 44 | 30 |
| 1609 | GAGTCCTGCASTATGAATATSATTT | 17 | 70 | 67 | 42 |
| 1610 | GAGTCCSGCAATATGAATSTAATTT | 29 | 83 | 76 | 40 |
| 1611 | GAGTCCSGCAATATGAASATAATTT | 23 | 95 | 60 | 41 |
| 1612 | GAGTCCTSCAATSTGAASATAATTT | −2 | 5 | 106 | 92 |
| 1613 | GAGTCCSGCAATSTGAATSTAATTT | 3 | 11 | 100 | 96 |
| 1614 | GAGTCSTGCAATSTGAATASAATTT | 8 | 2 | 94 | 101 |
| 1615 | GAGTSCTGCAATSTGAATATSATTT | 1 | 2 | 90 | 99 |
| 1633 | GTCTTCTSCCGAGTCSTGCAATA | 53 | 83 | 45 | 10 |
| 1634 | GTCTTCTGCCGSGTCCTGCAATA | 31 | 74 | 41 | 12 |
| 1635 | AGGTCTTCSGCCGAGTCCSGCAATA | 23 | N/A | 43 | N/A |
| 1617 | CCTTTCTCSCGAAGGTCTTCSGCCG | 49 | 89 | 24 | 9 |
| 1618 | CTTTCTCSCGAAGGTSTTCTGCC | 46 | 82 | 35 | 11 |
| 1619 | TTTCTCTSGAAGGTCSTCTGCCG | 42 | 80 | 40 | 12 |
| 1620 | TCTCTCGSACACACGSACACATG | 74 | 103 | 5 | 1 |
| 1589 | AATCCAATTASGAGAGAGTGASGGG | 7 | 32 | 107 | 47 |
| 1590 | GAGTCCSGCAATASGAATATAATTT | 23 | 63 | 76 | 47 |
| 1591 | GCACACASGCTCACACAGSGAGCCA | 45 | 86 | 11 | 2 |
| 1636 | GT*C*C*TGCSATATGAASATAAT | 18 | 53 | 75 | 74 |
| 1637 | GT*C*C*TSCAATATGSATATAAT | 4 | 2 | 130 | 105 |
| 1638 | GT*C*C*TGCSATATGSATATAAT | 24 | 41 | 75 | 68 |
| 1639 | GTCTTCTSCCGAGT*C*S*T*GCAATA | 26 | 67 | 60 | 30 |
| 1640 | GT*CT*TC*TGCCGSGTCCTGCAATA | 33 | 63 | 36 | 17 |
| 1641 | GTCTTCTGCC*G*S*G*TCCTGCAATA | 21 | 91 | 34 | 23 |
| 1642 | CCTTTCTCSCGAAGGTCT*T*C*SGCCG | 40 | 77 | 28 | 14 |
| 1643 | CCTTTCTCSCGAAGGTCTT*C*S*G*CCG | 40 | 77 | 21 | 15 |
| 1644 | CTTTCTCSCGAAGG*T*S*T*TCTGCC | 28 | 46 | 60 | 36 |
| 1645 | GC*A*CA*C*ASGCTCACASAGAGAGC | 30 | 73 | 22 | 4 |
| 1646 | GCACAC*A*S*G*CTCACASAGAGAGC | 12 | 37 | 29 | 11 |
| 1647 | TC*TC*TC*GSACACACGSACACATG | 40 | 90 | 15 | 3 |
| 1648 | TCTCTCGSACACACGSA*CA*CA*TG | 58 | 108 | 19 | 7 |
| 1649 | GTCTTCTS^{∧}CCGAGTCS^{∧}TGCAATA | 26 | 71 | 19 | 21 |

^{#}Unless otherwise noted, each of the nucleosides of antisense oligonucleotides shown in Table 30 are modified nucleosides with 2′-O-(2-methoxyethyl) (2′-MOE) sugar moieties and each “C” is replaced with a 5-methylcytosine (5-MeC). * indicates presence of phosphodiester linkage. All other internucleoside linkages are phosphorothioate linkages. A spacer as indicated by S is not a nucleoside. S represents a spacer of Formula (Iia′) as disclosed herein. S^{∧} represents a spacer of Formula (IIIa′) as disclosed herein.

TABLE 31A
Performance of STMN2 AONs (STMN2 oligonucleotides without spacers).

| | | QPCR potency in | | QPCR potency in | |
|---|---|---|---|---|---|
| | | hMN STMN2 FL | | hMN STMN2 cryptic | |
| SEQ ID | Oligonucleotide Sequence^{#} | 50 nM | 200 nM | 50 nM | 200 nM |
| NO: | (5′ to 3′) | Mean | Mean | Mean | Mean |
| 144 | AATCCAATTAAGAGAGAGTGATGGG | 2 | 23 | 71 | 49 |
| 146 | AAAATCCAATTAAGAGAGAGTGATG | 11 | 19 | 45 | 36 |
| 150 | TTTAAAAATCCAATTAAGAGAGAGT | 43.7 | 46.7 | 38.7 | 17.7 |
| 169 | CCTGCAATATGAATATAATTTTAAA | 36.3 | 72.3 | 45.3 | 11.7 |
| 170 | TCCTGCAATATGAATATAATTTTAA | 28.3 | 86.3 | 69.3 | 25.3 |
| 171 | GTCCTGCAATATGAATATAATTTTA | 30.7 | 85.0 | 56.3 | 12.3 |
| 172 | AGTCCTGCAATATGAATATAATTTT | 33.0 | 79.3 | 54.7 | 15.7 |
| 173 | GAGTCCTGCAATATGAATATAATTT | 29.0 | 81.5 | 61.3 | 21.0 |
| 197 | CCTTTCTCTCGAAGGTCTTCTGCCG | 26.7 | 79.3 | 44.4 | 12.1 |
| 237 | GCACACATGCTCACACAGAGAGCCA | 46 | 80 | 7 | 1 |
| 252 | CTCTCTCGCACACACGCACACATGC | 37.6 | 69.6 | 19.0 | 2.3 |
| 1343 | AATCCAATTAAGAGAGAGTGATG | 7 | 15 | 56 | 33 |
| 1346 | GTCCTGCAATATGAATATAATTT | 67.3 | 98.0 | 49.3 | 10.3 |
| 1347 | GTCTTCTGCCGAGTCCTGCAATA | 12.5 | 40.2 | 55.7 | 20.8 |
| 1348 | GCACACATGCTCACACAGAGAGC | 45.6 | 89.5 | 11.6 | 0.7 |
| 1351 | AATCCAATTAAGAGAGAGTGA | 10.0 | 12.0 | 69.0 | 35.0 |
| 1353 | GTCCTGCAATATGAATATAAT | 48.2 | 100.5 | 47.2 | 18.3 |
| 1353 | GT*CC*TG*CAATATGAA*TA*TA* AT | 36.4 | 84.3 | 64.0 | 32.8 |
| 1355 | CCTGCAATATGAATATAATTT | 50.0 | 79.0 | 21.5 | 7.0 |
| 1421 | CCTGCAATATGAATATAATTTTA | 44.0 | 120.0 | 32.0 | 8.0 |
| 1422 | TGCAATATGAATATAATTTTAAA | 43.9 | 80.7 | 40.5 | 24.0 |
| 1423 | CTGCAATATGAATATAATTTTAA | 48.0 | 88.7 | 38.3 | 10.7 |
| 1424 | TGCAATATGAATATAATTTTA | 40.0 | 79.0 | 33.0 | 15.0 |
| 1425 | TCCTGCAATATGAATATAATTTT | 39.0 | 95.8 | 40.6 | 12.0 |
| 1426 | CTGCAATATGAATATAATTTT | 45.5 | 85.2 | 39.4 | 12.6 |
| 1427 | AGTCCTGCAATATGAATATAATT | 39.7 | 76.0 | 42.3 | 13.3 |
| 1428 | TCCTGCAATATGAATATAATT | 43.0 | 91.5 | 42.8 | 10.0 |
| 1429 | TTTCTCTCGAAGGTCTTCTGCCG | 49.5 | 85.5 | 40.9 | 9.5 |
| 1430 | CCTTTCTCTCGAAGGTCTTCTGC | 41.5 | 98.2 | 27.5 | 5.9 |
| 1431 | CTTTCTCTCGAAGGTCTTCTGCC | 32.6 | 83.3 | 40.6 | 12.6 |
| 1432 | CTCTCGCACACACGCACACATGC | 34.0 | 51.8 | 25.5 | 3.1 |
| 1433 | CTCTCTCGCACACACGCACACAT | 20.2 | 60.8 | 6.5 | 2.9 |
| 1434 | TCTCTCGCACACACGCACACATG | 43.3 | 77.5 | 18.3 | 2.7 |
| 1435 | CTCTCGCACACACGCACACAT | 33.0 | 83.4 | 22.6 | 3.7 |
| 1650 | CT*C*TC*T*CGCACACACGCACACATGC | 26.6 | 68.8 | 40.3 | 13.3 |
| 1651 | TC*TC*TC*GCACACACGCACACATG | 46.1 | 91.0 | 32.4 | 8.9 |
| 1652 | TTTCTCTCGAAGGTCTTCTGC | 26.0 | 75.9 | 49.4 | 8.9 |
| 1653 | AAAATCCAATTAAGAGAGAGTGA | 15.0 | 19.0 | 49.0 | 29.0 |
| 1654 | AAATCCAATTAAGAGAGAGTGAT | 12.0 | 18.0 | 55.0 | 31.0 |
| 1655 | TAAAAATCCAATTAAGAGAGAGT | 32.0 | 42.0 | 37.0 | 24.0 |
| 1656 | TTTAAAAATCCAATTAAGAGAGA | 25.0 | 32.0 | 37.0 | 29.0 |
| 1657 | TTAAAAATCCAATTAAGAGAGAG | 18.0 | 20.0 | 33.0 | 19.0 |
| 1658 | TAAAAATCCAATTAAGAGAGA | 21.7 | 52.0 | 60.0 | 42.0 |
| 1659 | CC*T*T*TCTCTCGAAGGTCTTCTGCCG | 40.0 | 99.7 | 35.5 | 13.8 |
| 1660 | GCACACATGCTCACACA*GA*GA*GC | 40.8 | 85.1 | 12.9 | 3.4 |
| 1661 | GC*A*CA*C*ATGCTCACACAGAGAGC | 38.2 | 81.0 | 26.0 | 4.4 |

^{#}Unless otherwise noted, each of the nucleosides of antisense oligonucleotides shown in Table 31A are modified nucleosides with 2′-O-(2-methoxyethyl) (2′-MOE) sugar moieties and each “C” is replaced with a 5-methylcytosine (5-MeC). * indicates presence of phosphodiester linkage. All other internucleoside linkages are phosphorothioate linkages.

TABLE 31B
Performance of STMN2 AONs with one, two, or three spacers (as measured by restoration
of full length (FL) STMN2).

| | | Normalized | Rq | Normalized | Rq |
|---|---|---|---|---|---|
| SEQ | Oligonucleotide Sequence* | 200 nM ASO- | 200 nM ASO | 500 nM ASO | 500 nM ASO |
| ID NO: | (5′ to 3′) | STMN2 FL | STMN2 FL | STMN2 FL | STMN2 FL |
| 10655 | CTCTCTCGCACACACGCACACATGCTC | 203.68 | 1.55 | 145.43 | 1.19 |
| 10656 | CTCTCTSGCACACSCGCACASATGCTC | 83.90 | 0.82 | 60.07 | 0.68 |
| 10657 | GAGTCCTGCAATATGAATATAATTTTA | 44.36 | 0.58 | 69.10 | 0.73 |
| 10658 | GAGTCCSGCAATASGAATATSATTTTA | 5.56 | 0.34 | −0.26 | 0.31 |
| 1620 | TCTCTCGSACACACGSACACATG | 72.43 | 0.79 | 53.69 | 0.64 |
| 10660 | TCoTCoTCGSACACACGSACACATG | 69.29 | 0.76 | 52.48 | 0.63 |
| 10661 | TCTCTCGSACACACGSAoCAOCAOTG | 51.35 | 0.62 | 31.71 | 0.47 |
| 10662 | GCoAoCAoCoASGCTCACASAGAGAGC | 69.24 | 0.79 | 41.40 | 0.60 |
| 10663 | GCACACoAoSoGoCTCACASAGAGAGC | 59.60 | 0.72 | 41.43 | 0.60 |
| 1602 | GCACACASGCTCACASAGAGAGC | 71.29 | 0.80 | 42.77 | 0.61 |
| 1618 | CTTTCTCSCGAAGGTSTTCTGCC | 98.11 | 0.99 | 37.50 | 0.50 |
| 1619 | TTTCTCTSGAAGGTCSTCTGCCG | 75.63 | 0.81 | 31.19 | 0.45 |
| 10667 | CTTTCTCSCGAAGGoToSoToTCTGCC | 39.99 | 0.52 | 18.62 | 0.34 |
| 1633 | GTCTTCTSCCGAGTCSTGCAATA | 47.05 | 0.54 | 14.65 | 0.25 |
| 10669 | GTCTTCTSCCGAGToCoSoTOGCAATA | 54.26 | 0.65 | 51.66 | 0.63 |

*Unless otherwise noted, each of the nucleosides of antisense oligonucleotides shown in Table 31B are modified nucleosides with 2′-O-(2-methoxyethyl) (2′-MOE) sugar moieties and each “C” is replaced with a 5-methylcytosine (5-MeC). “o” indicates presence of phosphodiester linkage. All other internucleoside linkages are phosphorothioate linkages. A spacer as indicated by S is not a nucleoside. S represents a spacer of Formula (Iia′) as disclosed herein.;

TABLE 31C
Performance of STMN2 AONs with one, two, or three spacers (as measured by reduction of
STMN2 Cryptic exon).

| | | Normalized | Rq | Normalized | Rq |
|---|---|---|---|---|---|
| | | 200 nM ASO- | 200 nM ASO | 500 nM ASO | 500 nM ASO |
| SEQ ID | Oligonucleotide Sequence* | STMN2 | STMN2 | STMN2 | STMN2 |
| NO: | (5′ to 3′) | Cryptic | Cryptic | Cryptic | Cryptic |
| 10655 | CTCTCTCGCACACACGCACACATGCTC | −0.99 | 0.93 | −1.17 | 0.24 |
| 10656 | CTCTCTSGCACACSCGCACASATGCTC | 0.39 | 6.32 | −1.19 | 0.17 |
| 10657 | GAGTCCTGCAATATGAATATAATTTTA | 9.99 | 43.73 | −0.08 | 4.47 |
| 10658 | GAGTCCSGCAATASGAATATSATTTTA | 23.34 | 95.76 | 12.79 | 54.62 |
| 1620 | TCTCTCGSACACACGSACACATG | 1.60 | 25.68 | 6.54 | 100.04 |
| 10660 | TCoTCoTCGSACACACGSACACATG | 1.87 | 29.84 | 7.90 | 120.43 |
| 10661 | TCTCTCGSACACACGSAoCAOCAOTG | 7.94 | 121.09 | 19.57 | 295.96 |
| 10662 | GCoAoCAoCoASGCTCACASAGAGAGC | 4.49 | 65.78 | 22.89 | 330.02 |
| 10663 | GCACACoAoSoGoCTCACASAGAGAGC | 10.69 | 154.87 | 24.42 | 352.10 |
| 1602 | GCACACASGCTCACASAGAGAGC | 2.29 | 34.28 | 9.71 | 140.78 |
| 1618 | CTTTCTCSCGAAGGTSTTCTGCC | 15.39 | 284.63 | 20.65 | 381.44 |
| 1619 | TTTCTCTSGAAGGTCSTCTGCCG | 68.67 | 1265.76 | 7.87 | 146.12 |
| 10667 | CTTTCTCSCGAAGGoToSoToTCTGCC | 33.07 | 610.23 | 41.91 | 772.91 |
| 1633 | GTCTTCTSCCGAGTCSTGCAATA | 12.61 | 166.34 | 73.59 | 966.16 |
| 10669 | GTCTTCTSCCGAGToCoSoTOGCAATA | 33.65 | 433.07 | 72.06 | 926.00 |

*Unless otherwise noted, each of the nucleosides of antisense oligonucleotides shown in Table 31C are modified nucleosides with 2′-O-(2-methoxyethyl) (2′-MOE) sugar moieties and each “C” is replaced with a 5-methylcytosine (5-MeC). “o” indicates presence of phosphodiester linkage. All other internucleoside linkages are phosphorothioate linkages. A spacer as indicated by S is not a nucleoside. S represents a spacer of Formula (Iia′) as disclosed herein.

TABLE 31D
Performance of STMN2 AONs with LNA modifications.

| | | STMN2 FL | | | |
|---|---|---|---|---|---|
| | | 200 nM | 200 nM | 50 nM | 50 nM |
| SEQ ID | Oligonucleotide Sequence* | Normalized | Normalized | Normalized | Normalized |
| NO: | (5′ to 3′) | RQ | RQ STD | RQ | RQ STD |
| 1620 | TCTCTCGSACACACGSACACATG | 72.79 | 6.953 | 44.89 | 3.64 |
| 10815 | TCTCTC[LNA-G]S[LNA-A]CACACGSACACATG | 85.67 | 3.037 | 59.83 | 1.924 |
| 10816 | TCTCTCGSACACAC[LNA-G]S[LNA-A]CACATG | 77.56 | 2.304 | 61.92 | 7.248 |
| 1633 | GTCTTCTSCCGAGTCSTGCAATA | 49.66 | 5.794 | 21.1 | 1.551 |
| 10817 | GTCTTC[LNA-T]S[LNA-C]CGAGTCSTGCAATA | 51.12 | 11.34 | 19.65 | 1.85 |
| 10818 | GTCTTCTSCCGAGT[LNA-C]S[LNA-T]GCAATA | 50.83 | 6.278 | 23.95 | 2.508 |
| 1618 | CTTTCTCSCGAAGGTSTTCTGCC | 67 | 22.27 | 31.26 | 17.12 |
| 10819 | CTTTCT[LNA-C]S[LNA-C]GAAGGTSTTCTGCC | 74.7 | 21.25 | 43.38 | 27.35 |
| 10820 | CTTTCTCSCGAAGG[LNA-T]S[LNA-T]TCTGCC | 63.52 | 26.61 | 37.99 | 20.32 |

TABLE 31E
Performance of STMN2 AONs with LNA modifications.

| | | STMN2 Cryptic | | | |
|---|---|---|---|---|---|
| | | 200 nM | 200 nM | 50 nM | 50 nM |
| SEQ ID | Oligonucleotide Sequence* | Normalized | Normalized | Normalized | Normalized |
| NO: | (5′ to 3′) | RQ | RQ STD | RQ | RQ STD |
| 1620 | TCTCTCGSACACACGSACACATG | 4.043 | 1.967 | 26.9 | 6.989 |
| 10815 | TCTCTC[LNA-G]S[LNA-A]CACACGSACACATG | 0.8398 | 0.7405 | 16.75 | 10.8 |
| 10816 | TCTCTCGSACACAC[LNA-G]S[LNA-A]CACATG | 3.702 | 3.39 | 10.58 | 5.852 |
| 1633 | GTCTTCTSCCGAGTCSTGCAATA | 24.71 | 1.319 | 55.5 | 8.628 |
| 10817 | GTCTTC[LNA-T]S[LNA-C]CGAGTCSTGCAATA | 17.95 | 3.209 | 51.88 | 1.863 |
| 10818 | GTCTTCTSCCGAGT[LNA-C]S[LNA-T]GCAATA | 21.85 | 3.382 | 63.6 | 11.8 |
| 1618 | CTTTCTCSCGAAGGTSTTCTGCC | 24.66 | 9.5 | 72.62 | 11.89 |
| 10819 | CTTTCT[LNA-C]S[LNA-C]GAAGGTSTTCTGCC | 18.21 | 5.833 | 45.17 | 9.862 |
| 10820 | CTTTCTCSCGAAGG[LNA-T]S[LNA-T]TCTGCC | 22.84 | 4.195 | 63.19 | 11.64 |

*Unless otherwise noted, each of the nucleosides of antisense oligonucleotides shown in Tables 31D and 31E are modified nucleosides with 2′-O-(2-methoxyethyl) (2′-MOE) sugar moieties, each “C” is replaced with a 5-methylcytosine (5-MeC), and all internucleoside linkages are phosphorothioate linkages. Spacer as indicated by S is a nucleoside-replacement group and is not a nucleoside. S represents a spacer of Formula (Iia′) as disclosed herein. [LNA-X] refers to a locked nucleic acid with a corresponding base X (e.g., LNA-A refers to a locked nucleic acid with an adenine nucleobase, LNA-G refers to a locked nucleic acid with a guanine nucleobase, LNA-T refers to a locked nucleic acid with a thymine nucleobase, and LNA-C refers to a locked nucleic acid with a 5-methylcytosine nucleobase).

### Example 8: Design and Selection of KCNQ2 Oligonucleotides

KCNQ2 AONs that target a KCNQ2 transcript are designed and tested to identify KCNQ2 AONs capable of reducing quantity of mis-spliced KCNQ2 transcripts. Such KCNQ2 AONs include a subset of the KCNQ2 parent oligonucleotides represented by any of SEQ ID NOs: 1676-1851, SEQ ID NOs: 2028-2529, SEQ ID NOs: 3046-3221, SEQ ID NOS: 3398-3899, or SEQ ID NOs: 4402-4409. The KCNQ2 parent oligonucleotides are 25 nucleosides in length. Each of the nucleosides of the KCNQ2 parent oligonucleotides are modified nucleosides with 2′-MOE sugar moieties, and each “C” is replaced with a 5-MeC. Additionally, each of the internucleoside linkages between the nucleosides of the KCNQ2 oligonucleotides are phosphorothioate internucleoside linkages.

Generally, the length of the KCNQ2 antisense oligonucleotides are 25 oligonucleotide units in length. However, variants of the KCNQ2 antisense oligonucleotides were also designed with varying lengths (e.g., 23mers). Examples of these variant KCNQ2 antisense oligonucleotides were designed to include a subset of the sequences of SEQ ID NOs: 3046-3221 and SEQ ID NOs: 3398-3899.

TABLE 32
KCNQ2 AONs (including KCNQ2 parent oligo-
nucleotides and KCNQ2 oligonucleotides with
two spacers)

| | Oligonucleotide Sequence* (where S |
|---|---|
| | indicates presence of a Spacer) |
| SEQ ID No: | (5′ to 3′) |
| 4469 | CACCACSGACTCCATCCCSCCACCC |
| 4470 | CTCGTTSACACCTGATGGSCTCCAG |
| 4471 | AGTCACSAGAAGAAGCACSGAGAAG |
| 4472 | GCTCTTSAAGCAAACCCCSGGCAGG |
| 4473 | CACCACTGSCTCCATCCCSCCACCC |
| 4474 | CTCGTTCASACCTGATGGSCTCCAG |
| 4475 | AGTCACGASAAGAAGCACSGAGAAG |
| 4476 | GCTCTTGASGCAAACCCCSGGCAGG |
| 3117 | TCTTGAAGCAAACCCCAGGCAGG |
| 3119 | GCTCTTGAAGCAAACCCCAGGCA |
| 3118 | CTCTTGAAGCAAACCCCAGGCAG |
| 3142 | CTGACTCCATCCCTCCACCCAGG |
| 3144 | CACTGACTCCATCCCTCCACCCA |
| 3143 | ACTGACTCCATCCCTCCACCCAG |
| 3153 | GAGCCACCACTGACTCCATCCCT |
| 3152 | CAGAGCCACCACTGACTCCATCC |
| 3177 | AGAGCCACCACTGACTCCATCCC |
| 3117 | CGTTCACACCTGATGGGCTCCAG |
| 3179 | CTCGTTCACACCTGATGGGCTCC |
| 3178 | TCGTTCACACCTGATGGGCTCCA |
| 3186 | AGGGAGGCTCGTTCACACCTGAT |
| 3188 | GGAGGGAGGCTCGTTCACACCTG |
| 3187 | GAGGGAGGCTCGTTCACACCTGA |
| 3216 | TCACGAGAAGAAGCACCGAGAAG |
| 3218 | AGTCACGAGAAGAAGCACCGAGA |
| 3217 | GTCACGAGAAGAAGCACCGAGAA |
| 4477 | TCTTGAASCAAACCCSAGGCAGG |
| 4478 | GCTCTTGSAGCAAACSCCAGGCA |
| 4479 | CTCTTGASGCAAACCSCAGGCAG |
| 4480 | CTGACTCSATCCCTCSACCCAGG |
| 4481 | CACTGACSCCATCCCSCCACCCA |
| 4482 | ACTGACTSCATCCCTSCACCCAG |
| 4483 | GAGCCACSACTGACTSCATCCCT |
| 4484 | CAGAGCCSCCACTGASTCCATCC |
| 4485 | AGAGCCASCACTGACSCCATCCC |
| 4486 | CGTTCACSCCTGATGSGCTCCAG |
| 4487 | CTCGTTCSCACCTGASGGGCTCC |
| 4488 | TCGTTCASACCTGATSGGCTCCA |
| 4489 | AGGGAGGSTCGTTCASACCTGAT |
| 4490 | GGAGGGASGCTCGTTSACACCTG |
| 4491 | GAGGGAGSCTCGTTCSCACCTGA |
| 4492 | TCACGAGSAGAAGCASCGAGAAG |
| 4493 | AGTCACGSGAAGAAGSACCGAGA |
| 4494 | GTCACGASAAGAAGCSCCGAGAA |
| 4495 | TCAACASCATGACCACCASCACCAC |
| 4496 | ACCAGASGCATCACCATCSCCACCA |
| 4497 | GAGACASAGGAAGCCGATSTACCAG |
| 4498 | AAGTACSCCAGGAACGAGSCCAGGA |
| 4499 | ACAGGGSCAGGGGTGTATSAGCAGG |
| 4500 | TCGACCSCAAGCCATCATSACCACG |
| 4501 | TCAACACCSTGACCACCASCACCAC |
| 4502 | ACCAGAAGSATCACCATCSCCACCA |
| 4503 | GAGACAAASGAAGCCGATSTACCAG |
| 4504 | AAGTACACSAGGAACGAGSCCAGGA |
| 4505 | ACAGGGACSGGGGTGTATSAGCAGG |
| 4506 | TCGACCACSAGCCATCATSACCACG |
| 3489 | AACACCATGACCACCATCACCAC |
| 3491 | TCAACACCATGACCACCATCACC |
| 3490 | CAACACCATGACCACCATCACCA |
| 3534 | CAGAAGCATCACCATCACCACCA |
| 3536 | ACCAGAAGCATCACCATCACCAC |
| 3535 | CCAGAAGCATCACCATCACCACC |
| 3563 | GACGGCACCACCATCATGACCAC |
| 3565 | CAGACGGCACCACCATCATGACC |
| 3564 | AGACGGCACCACCATCATGACCA |
| 3621 | GGCCAGGATGAGACAAAGGAAGC |
| 3623 | GAGGCCAGGATGAGACAAAGGAA |
| 3522 | AGGCCAGGATGAGACAAAGGAAG |
| 3636 | GTACACCAGGAACGAGGCCAGGA |
| 3638 | AAGTACACCAGGAACGAGGCCAG |
| 3637 | AGTACACCAGGAACGAGGCCAGG |
| 3666 | AAAGTGGTCGTTCTCCCCCTTCT |
| 3668 | TCAAAGTGGTCGTTCTCCCCCTT |
| 3667 | CAAAGTGGTCGTTCTCCCCCTTC |
| 3753 | AGGGACAGGGGTGTATCAGCAGG |
| 3755 | ACAGGGACAGGGGTGTATCAGCA |
| 3754 | CAGGGACAGGGGTGTATCAGCAG |
| 3845 | GACCACAAGCCATCATGACCACG |
| 3847 | TCGACCACAAGCCATCATGACCA |
| 3846 | CGACCACAAGCCATCATGACCAC |
| 4507 | AACACCASGACCACCSTCACCAC |
| 4508 | TCAACACSATGACCASCATCACC |
| 4509 | CAACACCSTGACCACSATCACCA |
| 4510 | CAGAAGCSTCACCATSACCACCA |
| 4511 | ACCAGAASCATCACCSTCACCAC |
| 4512 | CCAGAAGSATCACCASCACCACC |
| 4513 | GACGGCASCACCATCSTGACCAC |
| 4514 | CAGACGGSACCACCASCATGACC |
| 4515 | AGACGGCSCCACCATSATGACCA |
| 4516 | GGCCAGGSTGAGACASAGGAAGC |
| 4517 | GAGGCCASGATGAGASAAAGGAA |
| 4518 | AGGCCAGSATGAGACSAAGGAAG |
| 4519 | GTACACCSGGAACGASGCCAGGA |
| 4520 | AAGTACASCAGGAACSAGGCCAG |
| 4521 | AGTACACSAGGAACGSGGCCAGG |
| 4522 | AAAGTGGSCGTTCTCSCCCTTCT |
| 4523 | TCAAAGTSGTCGTTCSCCCCCTT |
| 4524 | CAAAGTGSTCGTTCTSCCCCTTC |
| 4525 | AGGGACASGGGTGTASCAGCAGG |
| 4526 | ACAGGGASAGGGGTGSATCAGCA |
| 4527 | CAGGGACSGGGGTGTSTCAGCAG |
| 4528 | GACCACASGCCATCASGACCACG |
| 4529 | TCGACCASAAGCCATSATGACCA |
| 4530 | CGACCACSAGCCATCSTGACCAC |
| 1774 | CACTGACTCCATCCCTCCACCCAGG |
| 1777 | CACCACTGACTCCATCCCTCCACCC |
| 1780 | AGCCACCACTGACTCCATCCCTCCA |
| 1783 | CAGAGCCACCACTGACTCCATCCCT |
| 1786 | AGCCAGAGCCACCACTGACTCCATC |
| 1789 | TCCAGCCAGAGCCACCACTGACTCC |
| 1809 | CTCGTTCACACCTGATGGGCTCCAG |
| 1812 | AGGCTCGTTCACACCTGATGGGCTC |
| 1815 | GGGAGGCTCGTTCACACCTGATGGG |
| 1818 | GGAGGGAGGCTCGTTCACACCTGAT |
| 1843 | CGAGAAGAAGCACCGAGAAGAAAGG |
| 1848 | AGTCACGAGAAGAAGCACCGAGAAG |
| 1749 | GCTCTTGAAGCAAACCCCAGGCAGG |
| 3117 | TCTTGAAGCAAACCCCAGGCAGG |
| 3119 | GCTCTTGAAGCAAACCCCAGGCA |
| 3118 | CTCTTGAAGCAAACCCCAGGCAG |
| 3142 | CTGACTCCATCCCTCCACCCAGG |
| 3144 | CACTGACTCCATCCCTCCACCCA |
| 3143 | ACTGACTCCATCCCTCCACCCAG |
| 3151 | GAGCCACCACTGACTCCATCCCT |
| 3153 | CAGAGCCACCACTGACTCCATCC |
| 3152 | AGAGCCACCACTGACTCCATCCC |
| 3177 | CGTTCACACCTGATGGGCTCCAG |
| 3179 | CTCGTTCACACCTGATGGGCTCC |
| 3178 | TCGTTCACACCTGATGGGCTCCA |
| 3186 | AGGGAGGCTCGTTCACACCTGAT |
| 3188 | GGAGGGAGGCTCGTTCACACCTG |
| 3187 | GAGGGAGGCTCGTTCACACCTGA |
| 3216 | TCACGAGAAGAAGCACCGAGAAG |
| 3218 | AGTCACGAGAAGAAGCACCGAGA |
| 3217 | GTCACGAGAAGAAGCACCGAGAA |
| 2121 | TCAACACCATGACCACCATCACCAC |
| 2135 | CCACCACCATCACATCAACACCATG |
| 2166 | ACCAGAAGCATCACCATCACCACCA |
| 2177 | TGACCACCATCACCAGAAGCATCAC |
| 2195 | CAGACGGCACCACCATCATGACCAC |
| 2208 | AGCTCCTGAGAGGCAGACGGCACCA |
| 2223 | TACCAGGCAGTGACCAGCTCCTGAG |
| 2242 | GAGACAAAGGAAGCCGATGTACCAG |
| 2253 | GAGGCCAGGATGAGACAAAGGAAGC |
| 2268 | AAGTACACCAGGAACGAGGCCAGGA |
| 2283 | TCCCCCTTCTCTGCCAAGTACACCA |
| 2298 | TCAAAGTGGTCGTTCTCCCCCTTCT |
| 2307 | GCGTAGGTGTCAAAGTGGTCGTTCT |
| 2322 | CACCAGAGTGCATCCGCGTAGGTGT |
| 2342 | GACCACAACTCACCAGGCCCCACCA |
| 2385 | ACAGGGACAGGGGTGTATCAGCAGG |
| 2422 | ATGGAGCAGGCTCAGCCAGTGAGAG |
| 2434 | CCAAAGAGATGTATGGAGCAGGCTC |
| 2446 | CAGCAGGTGGCCCCAAAGAGATGTA |
| 2465 | CATCATGACCACGGGCAGCCAGCAG |
| 2477 | TCGACCACAAGCCATCATGACCACG |
| 2492 | AACCACCACCCCGCCTCGACCACAA |
| 3489 | AACACCATGACCACCATCACCAC |
| 3491 | TCAACACCATGACCACCATCACC |
| 3490 | CAACACCATGACCACCATCACCA |
| 3534 | CAGAAGCATCACCATCACCACCA |
| 3536 | ACCAGAAGCATCACCATCACCAC |
| 3535 | CCAGAAGCATCACCATCACCACC |
| 3563 | GACGGCACCACCATCATGACCAC |
| 3565 | CAGACGGCACCACCATCATGACC |
| 3564 | AGACGGCACCACCATCATGACCA |
| 3621 | GGCCAGGATGAGACAAAGGAAGC |
| 3623 | GAGGCCAGGATGAGACAAAGGAA |
| 3622 | AGGCCAGGATGAGACAAAGGAAG |
| 3636 | GTACACCAGGAACGAGGCCAGGA |
| 3638 | AAGTACACCAGGAACGAGGCCAG |
| 3637 | AGTACACCAGGAACGAGGCCAGG |
| 3666 | AAAGTGGTCGTTCTCCCCCTTCT |
| 3668 | TCAAAGTGGTCGTTCTCCCCCTT |
| 3667 | CAAAGTGGTCGTTCTCCCCCTTC |
| 3753 | AGGGACAGGGGTGTATCAGCAGG |
| 3755 | ACAGGGACAGGGGTGTATCAGCA |
| 3754 | CAGGGACAGGGGTGTATCAGCAG |
| 3845 | GACCACAAGCCATCATGACCACG |
| 3847 | TCGACCACAAGCCATCATGACCA |
| 3846 | CGACCACAAGCCATCATGACCAC |

*Unless otherwise noted, each of the nucleosides of antisense oligonucleotides shown in Table 32 are modified nucleosides with 2′-O-(2-methoxyethyl) (2′-MOE) sugar moieties, each “C” is replaced with a 5-methylcytosine (5-MeC), and all internucleoside linkages are phosphorothioate linkages. A spacer as indicated by S is not a nucleoside. S represents a spacer of Formula (Iia′) as disclosed herein.

### Example 9: Methods for Evaluating KCNQ2 Antisense Oligonucleotides

KCNQ2 antisense oligonucleotides were evaluated in iPSC derived human motor neurons (hMN). The cells were seeded in 96-well plates at a density of 50,000 cells/well. Antisense oligonucleotide (AON) to TDP43 was transfected with Endoporter (Gene Tools, Philomiath, OR, USA) to increase expression of exon 5 containing KCNQ2 transcript. Vehicle control consisted of motor neuron treatment with Endoporter alone. Positive controls included cells that were treated with TDP43 AON alone (“AON TDP43” or “TDP43 AON”).

TDP43 AON is a gapmer oligonucleotide and has the following sequence and chemistry:

(SEQ ID NO: 1669)
5′ A*A*G*G*C*T*T*C*A*T*A*T*T*G*T*A*C*T*T*T 3′

where *=phosphorothioate, underlined=DNA, other=2′-MOE RNA; each “C” is 5-MeC.

To evaluate KCNQ2 AON ability to restore Exon 5 inclusion in the presence of TDP-43 knockdown, antisense oligonucleotides to KCNQ2 were co-incubated with TDP43 AON in Endoporter in media before addition to the cells. After 72 hours, antisense oligonucleotides and Endoporter were washed out and replaced with fresh media. After 72 additional hours fresh media was replaced, and after 72 hours RNA was collected from the 96-well plates for RT-qPCR. RNA was isolated, cDNA generated, and multiplexed RT-qPCR assay performed with TaqMan probes for KCNQ2 exon 5 inclusion in the transcript and reference GAPDH quantification.

Transcript levels (e.g., KCNQ2 exon 5 containing transcript or TDP43 transcript) were detected by RT-qPCR using Taqman. Specifically, RT-qPCR was performed for detecting GAPDH using Thermofisher TaqMan Gene Expression Assay Hs03929097_g1. RT-qPCR was performed for detecting KCNQ2 exon 5 containing transcripts using custom Thermofisher TaqMan targeting exon junction 5/6.

Exon 5/6 Primers:
Forward:
(SEQ ID NO: 10652)
5′ GGGAGAACGACCACTTTGA 3′
Reverse:
(SEQ ID NO: 10653)
3′ GAAGAAGGAGACACCGATGAG 3′
Probe:
(SEQ ID NO: 10654)
5′ TAGCCAATGGTGGTCAGCGTGATC 3′

RT-qPCR was also performed for detecting KCNQ2 exon 5 containing transcripts using custom Thermofisher TaqMan targeting exon junction 4/5.

Exon 4/5 Primers:
Forward:
(SEQ ID NO: 10,821)
5′ GGATGATCCGCATGGAC 3′
Reverse:
(SEQ ID NO: 10,822)
3′ CCTTCTCTGCCAAGTACAC 3′
Probe:
(SEQ ID NO: 10,823)
5′ AGGCCAGGATGAGACAAAGGAAGC 3′

RT-qPCR was performed on Applied Biosystems® 7500 Real-time PCR systems. One cycle of reverse transcription was performed at a temperature of 50° C. for 5 min. One cycle of RT inactivation/initial denaturation was performed at a temperature of 95° C. for 20 seconds. Forty five cycles of amplification were performed at a temperature of 95° C. for 1 second followed by 60° C. for 20 seconds.

KCNQ2 Exon 5 inclusion (Ct) was normalized to GAPDH (deltaCt). To visualize the quantitative changes (e.g., % increase of KCNQ2 Exon 5 inclusion), the normalized KCNQ2 Exon 5 inclusion signal was further normalized to the vehicle (treated with Endoporter alone, deltadeltaCt). Relative quantity (RQ) of transcript level was calculated using the equation RQ=2^{-deltadeltaCt} and is used to describe the treatment condition comparison to normal, healthy levels (1.0).

RQ values for KCNQ2 Exon Junctions 5/6 and 4/5 were normalized using the following formula:

$$( ( RQAON - R Q T D P 43 ) / ( RQe ndo - RQTDP 43 ) ) * 100$$

Tables 33A, 33B, and 34 show the RT-qPCR results of KCNQ2 AONs with spacers and performance of KCNQ2 AONs without spacers in human motor neurons. KCNQ2 AONs (e.g., KCNQ2 oligonucleotides without spacers or with one or two spacers) were tested for their ability to increase or restore full-length KCNQ2 mRNA (i.e., mRNA from which full-length KCNQ2 is translated which include exon 5 in the transcript) levels. In some cases, KCNQ2 AONs with spacers increased full-length KCNQ2 mRNA (“KCNQ2 FL”). In some cases, KCNQ2 AONs without spacers increased full-length KCNQ2 mRNA (“KCNQ2 FL”). Specific AON sequences are labeled according to their corresponding SEQ ID NO.

As shown in Table 33A, a 200 nM dose of SEQ ID NO: 3621 (GGCCAGGATGAGACAAAGGAAGC with no spacers) rescued full length KCNQ2 mRNA to −0.2490%. A 500 nM dose of SEQ ID NO: 3621 (GGCCAGGATGAGACAAAGGAAGC with no spacers) rescued full length KCNQ2 mRNA to 27.870%. Comparatively, 200 nM dose of SEQ ID NO: 4516 (GGCCAGGSTGAGACASAGGAAGC with two spacers) rescued full length KCNQ2 mRNA to 4.1655%. A 500 nM dose of SEQ ID NO: 4516 (GGCCAGGSTGAGACASAGGAAGC with two spacers) rescued full length KCNQ2 mRNA to 59.333%. This indicates that the addition of spacers of SEQ ID NO: 4516 improves the performance in comparison to the KCNQ2 AON counterpart without spacers (e.g., SEQ ID NO: 3621).

Additionally shown in Table 33A, a 200 nM dose of SEQ ID NO: 3564 (AGACGGCACCACCATCATGACCA with no spacers) rescued full length KCNQ2 mRNA to −3.66%. A 500 nM dose of SEQ ID NO: 3564 (AGACGGCACCACCATCATGACCA with no spacers) rescued full length KCNQ2 mRNA to 16.627%. Comparatively, a 200 nM dose of SEQ ID NO: 4515 (AGACGGCSCCACCATSATGACCAT with two spacers) rescued full length KCNQ2 mRNA to 18.720%. A 500 nM dose of SEQ ID NO: 4515 (AGACGGCSCCACCATSATGACCAT with two spacers) rescued full length KCNQ2 mRNA to 26,191%. This indicates that the addition of spacers of SEQ ID NO: 4515 improves the performance in comparison to the KCNQ2 AON counterpart without spacers (e.g., SEQ ID NO: 3564).

Additionally shown in Table 33A, a 200 nM dose of SEQ ID NO: 3534 (CAGAAGCATCACCATCACCACCA with no spacers) rescued full length KCNQ2 mRNA to −4.824%. A 500 nM dose of SEQ ID NO: 3534 (CAGAAGCATCACCATCACCACCA with no spacers) rescued full length KCNQ2 mRNA to −4.822%. Comparatively, a 200 nM dose of SEQ ID NO: 4510 (CAGAAGCSTCACCATSACCACCA with two spacers) rescued full length KCNQ2 mRNA to −4.361%. A 500 nM dose of SEQ ID NO: 4510 (CAGAAGCSTCACCATSACCACCA with two spacers) rescued full length KCNQ2 mRNA to 31.426%. This indicates that the addition of spacers of SEQ ID NO: 4510 improves the performance in comparison to the KCNQ2 AON counterpart without spacers (e.g., SEQ ID NO: 3534).

Additionally shown in Table 33A, a 200 nM dose of SEQ ID NO: 3536 (ACCAGAAGCATCACCATCACCAC with no spacers) rescued full length KCNQ2 mRNA to −1.253%. A 500 nM dose of SEQ ID NO: 3536 (ACCAGAAGCATCACCATCACCAC with no spacers) rescued full length KCNQ2 mRNA to −4.846%. Comparatively, a 200 nM dose of SEQ ID NO: 4511 (ACCAGAASCATCACCSTCACCAC with two spacers) rescued full length KCNQ2 mRNA to 11.898%. A 500 nM dose of SEQ ID NO: 4511 (ACCAGAASCATCACCSTCACCAC with two spacers) rescued full length KCNQ2 mRNA to 27.103%. This indicates that the addition of spacers of SEQ ID NO: 4511 improves the performance in comparison to the KCNQ2 AON counterpart without spacers (e.g., SEQ ID NO: 3536).

Additionally shown in Table 33A, a 200 nM dose of SEQ ID NO: 3535 (CCAGAAGCATCACCATCACCACC with no spacers) rescued full length KCNQ2 mRNA to 0.349%. A 500 nM dose of SEQ ID NO: 3535 (CCAGAAGCATCACCATCACCACC with no spacers) rescued full length KCNQ2 mRNA to −2.195%. Comparatively, a 200 nM dose of SEQ ID NO: 4512 (CCAGAAGSATCACCASCACCACC with two spacers) rescued full length KCNQ2 mRNA to 12.326%. A 500 nM dose of SEQ ID NO: 4512 (CCAGAAGSATCACCASCACCACC with two spacers) rescued full length KCNQ2 mRNA to 18.047%. This indicates that the addition of spacers of SEQ ID NO: 4512 improves the performance in comparison to the KCNQ2 AON counterpart without spacers (e.g., SEQ ID NO: 3535).

Additionally shown in Table 33A, a 200 nM dose of SEQ ID NO: 3491 (TCAACACCATGACCACCATCACC with no spacers) rescued full length KCNQ2 mRNA to −5.7868%. A 500 nM dose of SEQ ID NO: 3491 (TCAACACCATGACCACCATCACC with no spacers) rescued full length KCNQ2 mRNA to −0.189%. Comparatively, a 200 nM dose of SEQ ID NO: 4508 (TCAACACSATGACCASCATCACC with two spacers) rescued full length KCNQ2 mRNA to 9.940%. A 500 nM dose of SEQ ID NO: 4508 (TCAACACSATGACCASCATCACC with two spacers) rescued full length KCNQ2 mRNA to 13.392%. This indicates that the addition of spacers of SEQ ID NO: 4508 improves the performance in comparison to the KCNQ2 AON counterpart without spacers (e.g., SEQ ID NO: 3491).

Additionally shown in Table 33A, a 200 nM dose of SEQ ID NO: 3490 (CAACACCATGACCACCATCACCA with no spacers) rescued full length KCNQ2 mRNA to −5.760%. A 500 nM dose of SEQ ID NO: 3490 (CAACACCATGACCACCATCACCA with no spacers) rescued full length KCNQ2 mRNA to 2.607%. Comparatively, a 200 nM dose of SEQ ID NO: 4509 (CAACACCSTGACCACSATCACCA with two spacers) rescued full length KCNQ2 mRNA to −3.772%. A 500 nM dose of SEQ ID NO: 4509 (CAACACCSTGACCACSATCACCA with two spacers) rescued full length KCNQ2 mRNA to 29.909%. This indicates that the addition of spacers of SEQ ID NO: 4509 improves the performance in comparison to the KCNQ2 AON counterpart without spacers (e.g., SEQ ID NO: 3490).

Additionally shown in Table 33B, a 200 nM dose of SEQ ID NO: 3119 (GCTCTTGAAGCAAACCCCAGGCA with no spacers) rescued full length KCNQ2 mRNA to 1.091%. A 500 nM dose of SEQ ID NO: 3119 (GCTCTTGAAGCAAACCCCAGGCA with no spacers) rescued full length KCNQ2 mRNA to 0.847%. Comparatively, a 200 nM dose of SEQ ID NO: 4478 (GCTCTTGSAGCAAACSCCAGGCA with two spacers) rescued full length KCNQ2 mRNA to −5.0199%. A 500 nM dose of SEQ ID NO: 4478 (GCTCTTGSAGCAAACSCCAGGCA with two spacers) rescued full length KCNQ2 mRNA to 12.505%. This indicates that the addition of spacers of SEQ ID NO: 4478 improves the performance in comparison to the KCNQ2 AON counterpart without spacers (e.g., SEQ ID NO: 3119).

Additionally shown in Table 33B, a 200 nM dose of SEQ ID NO: 3122 (CTGACTCCATCCCTCCACCCAGGwith no spacers) rescued full length KCNQ2 mRNA to −3.1968%. A 500 nM dose of SEQ ID NO: 3122 (CTGACTCCATCCCTCCACCCAGG with no spacers) rescued full length KCNQ2 mRNA to −2.600%. Comparatively, a 200 nM dose of SEQ ID NO: 4480 (CTGACTCSATCCCTCSACCCAGG with two spacers) rescued full length KCNQ2 mRNA to 0.28284%. A 500 nM dose of SEQ ID NO: 4480 (CTGACTCSATCCCTCSACCCAGG with two spacers) rescued full length KCNQ2 mRNA to 10.232%. This indicates that the addition of spacers of SEQ ID NO: 4480 improves the performance in comparison to the KCNQ2 AON counterpart without spacers (e.g., SEQ ID NO: 3122).

Altogether, these results demonstrate that different KCNQ2 AONs including two spacers are capable of increasing KCNQ2-FL to levels that are close or comparable to their non-spacer counterparts.

TABLE 33A
Performance of Exon 5 KCNQ2 AONs

| Seq ID | Oligonucleotide Sequence* | 200 nM ASO-KCNQ2 Ex 5/6 | | 500 nM ASO KCNQ2 Ex 5/6 | |
|---|---|---|---|---|---|
| NO: | (5′ to 3′) | Normalized | RQ | Normalized | RQ |
| 2121 | TCAACACCATGACCACCATCACCAC | 8.325616 | 0.150071 | −5.77457 | 0.002139 |
| 3490 | CAACACCATGACCACCATCACCA | −5.76045 | 0.002287 | 2.607233 | 0.090077 |
| 4509 | CAACACCSTGACCACSATCACCA | −3.77247 | 0.023144 | 29.90924 | 0.376515 |
| 3491 | TCAACACCATGACCACCATCACC | −5.78681 | 0.002011 | −0.18977 | 0.060732 |
| 4508 | TCAACACSATGACCASCATCACC | 9.940254 | 0.167011 | 13.93271 | 0.208898 |
| 3489 | AACACCATGACCACCATCACCAC | −2.30475 | 0.038543 | −5.7947 | 0.001928 |
| 4507 | AACACCASGACCACCSTCACCAC | −1.71265 | 0.044755 | −5.74463 | 0.002453 |
| 4495 | TCAACASCATGACCACCASCACCAC | −2.72537 | 0.03413 | 8.421964 | 0.151082 |
| 4501 | TCAACACCSTGACCACCASCACCAC | −0.25086 | 0.060091 | 1.407007 | 0.077484 |
| 2135 | CCACCACCATCACATCAACACCATG | −19.0639 | 0.071713 | −28.2601 | 0.002939 |
| 2166 | ACCAGAAGCATCACCATCACCACCA | −3.0601 | 0.01759 | −2.36099 | 0.024278 |
| 3535 | CCAGAAGCATCACCATCACCACC | 0.349521 | 0.050206 | −2.19539 | 0.025862 |
| 4512 | CCAGAAGSATCACCASCACCACC | 12.32676 | 0.164778 | 18.04785 | 0.219505 |
| 3536 | ACCAGAAGCATCACCATCACCAC | −1.25378 | 0.034869 | −4.84679 | 0.000499 |
| 4511 | ACCAGAASCATCACCSTCACCAC | 11.89858 | 0.160682 | 27.10304 | 0.306125 |
| 3534 | CAGAAGCATCACCATCACCACCA | −4.82434 | 0.000714 | −4.82226 | 0.000734 |
| 4510 | CAGAAGCSTCACCATSACCACCA | −4.36192 | 0.005137 | 31.42647 | 0.347482 |
| 4496 | ACCAGASGCATCACCATCSCCACCA | 2.007292 | 0.066064 | −1.64228 | 0.031153 |
| 4502 | ACCAGAAGSATCACCATCSCCACCA | −1.79673 | 0.029676 | −2.31004 | 0.024765 |
| 2177 | TGACCACCATCACCAGAAGCATCAC | −28.2653 | 0.0029 | −19.2815 | 0.070085 |
| 2195 | CAGACGGCACCACCATCATGACCAC | −4.69235 | 0.00137 | 2.30004 | 0.072169 |
| 3564 | AGACGGCACCACCATCATGACCA | −3.66478 | 0.011775 | 10.29784 | 0.153148 |
| 4515 | AGACGGCSCCACCATSATGACCA | 18.7204 | 0.238428 | 26.19085 | 0.314068 |
| 3565 | CAGACGGCACCACCATCATGACC | 11.90306 | 0.169402 | 9.477831 | 0.144846 |
| 4514 | CAGACGGSACCACCASCATGACC | −3.8861 | 0.009534 | 10.74563 | 0.157682 |
| 3563 | GACGGCACCACCATCATGACCAC | 0.445321 | 0.05339 | 8.490921 | 0.134853 |
| 4513 | GACGGCASCACCATCSTGACCAC | −2.49403 | 0.023629 | 14.90492 | 0.199796 |
| 2208 | AGCTCCTGAGAGGCAGACGGCACCA | −28.4183 | 0.001756 | −16.699 | 0.089398 |
| 2223 | TACCAGGCAGTGACCAGCTCCTGAG | −28.4845 | 0.001261 | −28.2576 | 0.002958 |
| 2242 | GAGACAAAGGAAGCCGATGTACCAG | 4.549223 | 0.046631 | 22.22418 | 0.224454 |
| 4497 | GAGACASAGGAAGCCGATSTACCAG | 5.349911 | 0.054687 | 9.317288 | 0.094601 |
| 4503 | GAGACAAASGAAGCCGATSTACCAG | 14.35194 | 0.145253 | 31.10439 | 0.313795 |
| 2253 | GAGGCCAGGATGAGACAAAGGAAGC | 4.805941 | 0.08232 | 12.73105 | 0.159425 |
| 3522 | AGGCCAGGATGAGACAAAGGAAG | −1.31397 | 0.022778 | 34.54471 | 0.257518 |
| 4518 | AGGCCAGSATGAGACSAAGGAAG | 4.925139 | 0.08348 | 27.34078 | 0.301566 |
| 3623 | GAGGCCAGGATGAGACAAAGGAA | 5.710273 | 0.091118 | 14.34585 | 0.175136 |
| 4517 | GAGGCCASGATGAGASAAAGGAA | 20.78607 | 0.202769 | 24.68592 | 0.275736 |
| 3621 | GGCCAGGATGAGACAAAGGAAGC | −0.24903 | 0.033139 | 27.87085 | 0.306723 |
| 4516 | GGCCAGGSTGAGACASAGGAAGC | 4.165559 | 0.121048 | 59.33334 | 0.447587 |
| 2268 | AAGTACACCAGGAACGAGGCCAGGA | 1.649394 | 0.092803 | −7.87145 | 0.001263 |
| 3637 | AGTACACCAGGAACGAGGCCAGG | 5.673752 | 0.131496 | 12.60887 | 0.198175 |
| 4521 | AGTACACSAGGAACGSGGCCAGG | −3.74555 | 0.040932 | 17.13297 | 0.241673 |
| 3638 | AAGTACACCAGGAACGAGGCCAG | −6.43814 | 0.015044 | −4.46211 | 0.034043 |
| 4520 | AAGTACASCAGGAACSAGGCCAG | 7.768486 | 0.151636 | 22.29004 | 0.291257 |
| 3636 | GTACACCAGGAACGAGGCCAGGA | −7.86809 | 0.001295 | −0.75004 | 0.069733 |
| 4519 | GTACACCSGGAACGASGCCAGGA | 0.506936 | 0.081819 | 5.158879 | 0.126546 |
| 4498 | AAGTACSCCAGGAACGAGSCCAGGA | 7.834674 | 0.152273 | 4.585338 | 0.121031 |
| 4504 | AAGTACACSAGGAACGAGSCCAGGA | 2.254296 | 0.098619 | 14.06631 | 0.212188 |
| 2283 | TCCCCCTTCTCTGCCAAGTACACCA | −28.1841 | 0.003508 | −27.7819 | 0.006516 |
| 2298 | TCAAAGTGGTCGTTCTCCCCCTTCT | −6.56377 | 0.000837 | −6.55768 | 0.000895 |
| 3667 | CAAAGTGGTCGTTCTCCCCCTTC | −5.20966 | 0.013609 | −1.57848 | 0.047857 |
| 4524 | CAAAGTGSTCGTTCTSCCCCTTC | −2.76459 | 0.03667 | −6.37846 | 0.002585 |
| 3668 | TCAAAGTGGTCGTTCTCCCCCTT | −6.60007 | 0.000495 | −6.59773 | 0.000517 |
| 4523 | TCAAAGTSGTCGTTCSCCCCCTT | −2.95555 | 0.034869 | −6.6049 | 0.000449 |
| 3666 | AAAGTGGTCGTTCTCCCCCTTCT | −0.96274 | 0.053665 | −6.56497 | 0.000826 |
| 4522 | AAAGTGGSCGTTCTCSCCCTTCT | −6.63355 | 0.000179 | −6.63443 | 0.000171 |
| 2307 | GCGTAGGTGTCAAAGTGGTCGTTCT | −28.2064 | 0.003341 | −28.3379 | 0.002357 |
| 2322 | CACCAGAGTGCATCCGCGTAGGTGT | −24.562 | 0.030595 | −26.3803 | 0.016997 |
| 2342 | GACCACAACTCACCAGGCCCCACCA | 76.19122 | 0.784073 | 57.94834 | 0.647645 |
| 2385 | ACAGGGACAGGGGTGTATCAGCAGG | −0.593 | 0.048787 | −1.52436 | 0.039978 |
| 3754 | CAGGGACAGGGGTGTATCAGCAG | −2.19416 | 0.033643 | −5.5571 | 0.001837 |
| 4527 | CAGGGACSGGGGTGTSTCAGCAG | 2.146985 | 0.074701 | 5.988388 | 0.111033 |
| 3755 | ACAGGGACAGGGGTGTATCAGCA | −2.91064 | 0.026867 | −5.52224 | 0.002167 |
| 4526 | ACAGGGASAGGGGTGSATCAGCA | 7.735775 | 0.127559 | −0.90206 | 0.045864 |
| 3753 | AGGGACAGGGGTGTATCAGCAGG | −5.48914 | 0.00248 | −5.4487 | 0.002862 |
| 4525 | AGGGACASGGGTGTASCAGCAGG | −2.58444 | 0.029952 | 8.499329 | 0.134781 |
| 4499 | ACAGGGSCAGGGGTGTATSAGCAGG | 0.392099 | 0.058104 | −5.19929 | 0.005221 |
| 4505 | ACAGGGACSGGGGTGTATSAGCAGG | 3.138933 | 0.084083 | 2.89336 | 0.08176 |
| 2422 | ATGGAGCAGGCTCAGCCAGTGAGAG | −9.66659 | 0.06989 | 19.72048 | 0.324655 |
| 2434 | CCAAAGAGATGTATGGAGCAGGCTC | −12.4194 | 0.046026 | 2.501353 | 0.175377 |
| 2446 | CAGCAGGTGGCCCCAAAGAGATGTA | −17.6094 | 0.001032 | 1.740016 | 0.168777 |
| 2465 | CATCATGACCACGGGCAGCCAGCAG | −14.1249 | 0.031239 | −10.6075 | 0.061733 |
| 2477 | TCGACCACAAGCCATCATGACCACG | 5.246421 | 0.065339 | 8.169241 | 0.095061 |
| 3846 | CGACCACAAGCCATCATGACCAC | 5.997216 | 0.072974 | 1.215942 | 0.024354 |
| 4530 | CGACCACSAGCCATCSTGACCAC | 7.441267 | 0.087658 | −0.05528 | 0.011427 |
| 3847 | TCGACCACAAGCCATCATGACCA | 0.058253 | 0.012582 | −0.37611 | 0.008165 |
| 4529 | TCGACCASAAGCCATSATGACCA | 9.68244 | 0.110448 | −1.12929 | 0.000506 |
| 3845 | GACCACAAGCCATCATGACCACG | 8.700139 | 0.100459 | 5.620028 | 0.069138 |
| 4528 | GACCACASGCCATCASGACCACG | −1.08149 | 0.000992 | 2.175398 | 0.03411 |
| 4500 | TCGACCSCAAGCCATCATSACCACG | 4.464673 | 0.05739 | 0.740754 | 0.019522 |
| 4506 | TCGACCACSAGCCATCATSACCACG | 7.790323 | 0.091207 | 6.057462 | 0.073586 |
| 2492 | AACCACCACCCCGCCTCGACCACAA | −17.5167 | 0.001835 | −17.6258 | 0.000889 |

*Unless otherwise noted, each of the nucleosides of antisense oligonucleotides shown in Table 33A are modified nucleosides with 2′-O-(2-methoxyethyl) (2′-MOE) sugar moieties, each “C” is replaced with a 5-methylcytosine (5-MeC), and all internucleoside linkages are phosphorothioate linkages. A spacer as indicated by S is not a nucleoside. S represents a spacer of Formula (Iia′) as disclosed herein.

TABLE 33B
Performance of Additional KCNQ2 AONs

| SEQ ID | Oligonucleotide Sequence* | 200 nM ASO-KCNQ2 Ex 5/6 | | 500 nM ASO KCNQ2 Ex 5/6 | |
|---|---|---|---|---|---|
| NO: | (5′ to 3′) | Normalized | Rq | Normalized | Rq |
| 1749 | GCTCTTGAAGCAAACCCCAGGCAGG | 1.93649 | 0.068425 | 18.38289 | 0.285738 |
| 3118 | CTCTTGAAGCAAACCCCAGGCAG | 2.250466 | 0.072573 | 6.589694 | 0.129909 |
| 4479 | CTCTTGASGCAAACCSCAGGCAG | −2.14824 | 0.014452 | 6.53018 | 0.129123 |
| 3119 | GCTCTTGAAGCAAACCCCAGGCA | 1.091966 | 0.057266 | 0.847707 | 0.054038 |
| 4478 | GCTCTTGSAGCAAACSCCAGGCA | 5.091992 | 0.11012 | 12.50502 | 0.208071 |
| 3117 | TCTTGAAGCAAACCCCAGGCAGG | 4.754464 | 0.10566 | 6.094406 | 0.123365 |
| 4477 | TCTTGAASCAAACCCSAGGCAGG | 0.230027 | 0.045877 | 5.753291 | 0.118858 |
| 4472 | GCTCTTSAAGCAAACCCCSGGCAGG | 9.440352 | 0.167576 | 11.28187 | 0.191909 |
| 4476 | GCTCTTGASGCAAACCCCSGGCAGG | 2.996807 | 0.082435 | 10.75228 | 0.184911 |
| 1774 | CACTGACTCCATCCCTCCACCCAGG | 3.799346 | 0.068647 | 4.098425 | 0.07156 |
| 3143 | ACTGACTCCATCCCTCCACCCAG | −3.19909 | 0.000485 | −1.02264 | 0.021683 |
| 4482 | ACTGACTSCATCCCTSCACCCAG | 0.596296 | 0.037451 | −3.17927 | 0.000678 |
| 3144 | CACTGACTCCATCCCTCCACCCA | −3.20264 | 0.000451 | 5.184762 | 0.082141 |
| 4481 | CACTGACSCCATCCCSCCACCCA | −0.06587 | 0.031002 | 0.919638 | 0.0406 |
| 3142 | CTGACTCCATCCCTCCACCCAGG | −3.19678 | 0.000508 | −2.60029 | 0.006317 |
| 4480 | CTGACTCSATCCCTCSACCCAGG | 0.282843 | 0.034398 | 10.23219 | 0.131301 |
| 1777 | CACCACTGACTCCATCCCTCCACCC | 0.010805 | 0.000972 | 9.903886 | 0.100503 |
| 4469 | CACCACSGACTCCATCCCSCCACCC | 0.044825 | 0.001314 | 12.68526 | 0.128485 |
| 4473 | CACCACTGSCTCCATCCCSCCACCC | 8.672383 | 0.088113 | 2.887498 | 0.029913 |
| 1780 | AGCCACCACTGACTCCATCCCTCCA | −14.66 | 0.026601 | −16.5539 | 0.010182 |
| 1783 | CAGAGCCACCACTGACTCCATCCCT | −7.04437 | 0.096055 | 10.43101 | 0.243866 |
| 3152 | AGAGCCACCACTGACTCCATCCC | −1.35312 | 0.144193 | 1.344652 | 0.167012 |
| 4485 | AGAGCCASCACTGACSCCATCCC | −12.0146 | 0.054015 | 2.669907 | 0.178221 |
| 3153 | CAGAGCCACCACTGACTCCATCC | −6.40193 | 0.101489 | −3.85 | 0.123074 |
| 4484 | CAGAGCCSCCACTGASTCCATCC | −5.00699 | 0.113288 | 0.59525 | 0.160673 |
| 3151 | GAGCCACCACTGACTCCATCCCT | −6.59523 | 0.099854 | −9.62124 | 0.074259 |
| 4483 | GAGCCACSACTGACTSCATCCCT | −7.45797 | 0.092557 | −1.63694 | 0.141792 |
| 1786 | AGCCAGAGCCACCACTGACTCCATC | −10.7868 | 0.060179 | −16.1477 | 0.013703 |
| 1789 | TCCAGCCAGAGCCACCACTGACTCC | −10.175 | 0.065483 | −13.7112 | 0.034827 |
| 1809 | CTCGTTCACACCTGATGGGCTCCAG | −2.239 | 0.024616 | 2.317326 | 0.071087 |
| 3178 | TCGTTCACACCTGATGGGCTCCA | 0.786719 | 0.055476 | 3.932325 | 0.087559 |
| 4488 | TCGTTCASACCTGATSGGCTCCA | 1.958548 | 0.067428 | 5.1286 | 0.09976 |
| 3179 | CTCGTTCACACCTGATGGGCTCC | 1.297235 | 0.060683 | 4.81057 | 0.096516 |
| 4487 | CTCGTTCSCACCTGASGGGCTCC | 4.292697 | 0.091234 | −1.40928 | 0.033079 |
| 3177 | CGTTCACACCTGATGGGCTCCAG | 2.594567 | 0.073915 | −0.18297 | 0.045586 |
| 4486 | CGTTCACSCCTGATGSGCTCCAG | −4.46372 | 0.001926 | 4.237993 | 0.090676 |
| 4470 | CTCGTTSACACCTGATGGSCTCCAG | −4.58767 | 0.000662 | 5.551197 | 0.10407 |
| 4474 | CTCGTTCASACCTGATGGSCTCCAG | −4.55861 | 0.000958 | −4.58782 | 0.00066 |
| 1812 | AGGCTCGTTCACACCTGATGGGCTC | −0.35963 | 0.001218 | −0.36405 | 0.001171 |
| 1815 | GGGAGGCTCGTTCACACCTGATGGG | 2.023388 | 0.026268 | 9.707654 | 0.107046 |
| 1818 | GGAGGGAGGCTCGTTCACACCTGAT | 14.31439 | 0.229398 | 23.07648 | 0.311912 |
| 3187 | GAGGGAGGCTCGTTCACACCTGA | −6.86994 | 0.029903 | 30.5004 | 0.381824 |
| 4491 | GAGGGAGSCTCGTTCSCACCTGA | 19.74948 | 0.280581 | 5.058917 | 0.142238 |
| 3188 | GGAGGGAGGCTCGTTCACACCTG | 10.77789 | 0.196095 | 10.0375 | 0.189122 |
| 4490 | GGAGGGASGCTCGTTSACACCTG | 0.988222 | 0.103904 | 1.280966 | 0.106661 |
| 3186 | AGGGAGGCTCGTTCACACCTGAT | 9.70545 | 0.185995 | 10.49864 | 0.193465 |
| 4489 | AGGGAGGSTCGTTCASACCTGAT | 0.331036 | 0.097715 | −3.57754 | 0.060908 |
| 1843 | CGAGAAGAAGCACCGAGAAGAAAGG | −0.37951 | 0.001009 | 14.7896 | 0.160469 |
| 1848 | AGTCACGAGAAGAAGCACCGAGAAG | −10.1678 | 0.11889 | −7.62889 | 0.140594 |
| 3217 | GTCACGAGAAGAAGCACCGAGAA | −18.752 | 0.045507 | −13.6372 | 0.089231 |
| 4494 | GTCACGASAAGAAGCSCCGAGAA | −20.1269 | 0.033753 | −11.8098 | 0.104852 |
| 3218 | AGTCACGAGAAGAAGCACCGAGA | −20.7987 | 0.028009 | −22.4503 | 0.013891 |
| 4493 | AGTCACGSGAAGAAGSACCGAGA | 0.340654 | 0.208723 | −11.0172 | 0.111628 |
| 3216 | TCACGAGAAGAAGCACCGAGAAG | −14.1492 | 0.084854 | −9.95005 | 0.120751 |
| 4492 | TCACGAGSAGAAGCASCGAGAAG | −9.64096 | 0.123393 | −2.37911 | 0.185472 |
| 4471 | AGTCACSAGAAGAAGCACSGAGAAG | −21.0213 | 0.026107 | −12.3651 | 0.100106 |
| 4475 | AGTCACGASAAGAAGCACSGAGAAG | −16.5444 | 0.064379 | −3.15146 | 0.17887 |

*Unless otherwise noted, each of the nucleosides of antisense oligonucleotides shown in Table 33B are modified nucleosides with 2′-O-(2-methoxyethy1) (2′-MOE) sugar moieties, each “C” is replaced with a 5-methylcytosine (5-MeC), and all internucleoside linkages are phosphorothioate linkages. A spacer as indicated by S is not a nucleoside. S represents a spacer of Formula (Iia′) as disclosed herein.

TABLE 34
Performance of Additional KCNQ2 AONs

| | | Exons 4/5 | | | | | | Exons 5/6 | | | | | |
|---|---|---|---|---|---|---|---|---|---|---|---|---|---|
| | | 200 nM | | | 50 nM | | | 200 nM | | | 50 nM | | |
| | | Normalized | | | Normalized | | | Normalized | | | Normalized | | |
| SEQ ID | Oligonucleotide Sequence | Fold | | | Fold | | | Fold | | | Fold | | |
| NO: | (5′ to 3′) | Change | SD | N | Change | SD | N | Change | SD | N | Change | SD | N |
| 2135 | CCACCACCATCACATCAACACCATG | 13.23 | 0.96 | 3 | 7.50 | 4.18 | 3 | 42.41 | 4.38 | 3 | 15.17 | 6.78 | 3 |
| 2166 | ACCAGAAGCATCACCATCACCACCA | −4.71 | 2.24 | 3 | 2.41 | 3.65 | 3 | −0.22 | 4.73 | 3 | 5.06 | 2.84 | 3 |
| 3535 | CCAGAAGCATCACCATCACCACC | 5.68 | 5.11 | 3 | 9.12 | 4.89 | 3 | 5.24 | 7.00 | 3 | 14.45 | 4.65 | 3 |
| 4512 | CCAGAAGSATCACCASCACCACC | 5.22 | 1.02 | 3 | −0.15 | 2.22 | 3 | 8.81 | 3.04 | 3 | 2.65 | 2.02 | 3 |
| 3536 | ACCAGAAGCATCACCATCACCAC | 3.68 | 1.44 | 3 | 2.79 | 4.61 | 3 | 9.80 | 1.85 | 3 | 5.25 | 3.72 | 3 |
| 4511 | ACCAGAASCATCACCSTCACCAC | 6.48 | 1.38 | 3 | 5.54 | 3.56 | 3 | 8.84 | 0.75 | 3 | 8.44 | 3.46 | 3 |
| 3534 | CAGAAGCATCACCATCACCACCA | 4.62 | 2.40 | 3 | −0.82 | 3.17 | 3 | 9.74 | 2.87 | 3 | −1.62 | 5.29 | 3 |
| 4510 | CAGAAGCSTCACCATSACCACCA | 1.65 | 2.63 | 3 | 2.41 | 3.38 | 3 | 4.99 | 2.68 | 3 | 3.37 | 2.06 | 3 |
| 2177 | TGACCACCATCACCAGAAGCATCAC | 17.59 | 2.99 | 3 | 32.89 | 5.03 | 3 | 22.71 | 5.33 | 3 | 40.05 | 9.13 | 3 |
| 2195 | CAGACGGCACCACCATCATGACCAC | 5.96 | 4.82 | 3 | 4.08 | 0.45 | 3 | 11.89 | 4.65 | 3 | 10.42 | 3.55 | 3 |
| 3564 | AGACGGCACCACCATCATGACCA | −0.51 | 1.72 | 3 | 5.01 | 1.48 | 3 | 2.64 | 1.18 | 3 | 11.23 | 2.54 | 3 |
| 4515 | AGACGGCSCCACCATSATGACCA | 3.25 | 1.79 | 3 | −1.27 | 1.34 | 3 | 9.39 | 4.89 | 3 | 2.98 | 3.40 | 3 |
| 3565 | CAGACGGCACCACCATCATGACC | 8.65 | 3.02 | 3 | 3.15 | 4.16 | 3 | 12.63 | 2.82 | 3 | 8.02 | 6.60 | 3 |
| 4514 | CAGACGGSACCACCASCATGACC | −0.17 | 0.84 | 3 | 9.43 | 6.75 | 3 | 2.46 | 2.09 | 3 | 8.70 | 5.28 | 3 |
| 3563 | GACGGCACCACCATCATGACCAC | 9.17 | 2.02 | 3 | 3.36 | 3.16 | 3 | 17.48 | 3.76 | 3 | 8.77 | 3.65 | 3 |
| 4513 | GACGGCASCACCATCSTGACCAC | 9.12 | 3.32 | 3 | 4.82 | 0.84 | 3 | 18.11 | 2.37 | 3 | 12.82 | 3.80 | 3 |
| 2208 | AGCTCCTGAGAGGCAGACGGCACCA | 7.98 | 2.21 | 3 | 9.20 | 2.06 | 3 | 11.55 | 1.89 | 3 | 15.76 | 4.15 | 3 |
| 2223 | TACCAGGCAGTGACCAGCTCCTGAG | 0.76 | 6.67 | 3 | −0.45 | 6.01 | 3 | 9.87 | 10.00 | 3 | 4.21 | 4.42 | 3 |
| 2253 | GAGGCCAGGATGAGACAAAGGAAGC | −2.82 | 2.04 | 3 | −1.31 | 4.02 | 3 | 6.98 | 1.42 | 3 | 3.60 | 1.24 | 3 |
| 3522 | AGGCCAGGATGAGACAAAGGAAG | −5.75 | 2.12 | 3 | −6.11 | 3.24 | 3 | 0.43 | 3.74 | 3 | −1.44 | 5.54 | 3 |
| 4518 | AGGCCAGSATGAGACSAAGGAAG | 1.92 | 1.47 | 3 | −1.36 | 4.31 | 3 | 6.38 | 2.89 | 3 | 1.17 | 3.18 | 3 |
| 3623 | GAGGCCAGGATGAGACAAAGGAA | 3.10 | 2.76 | 3 | 0.79 | 2.00 | 3 | 13.38 | 1.72 | 3 | 3.00 | 4.06 | 3 |
| 4517 | GAGGCCASGATGAGASAAAGGAA | −0.24 | 2.71 | 3 | 0.94 | 1.49 | 3 | 3.44 | 1.21 | 3 | 2.71 | 3.62 | 3 |
| 3621 | GGCCAGGATGAGACAAAGGAAGC | −4.64 | 0.31 | 3 | −10.34 | 2.60 | 3 | 5.24 | 1.48 | 3 | −8.00 | 3.49 | 3 |
| 4516 | GGCCAGGSTGAGACASAGGAAGC | −2.99 | 2.86 | 3 | −0.62 | 1.30 | 3 | 1.30 | 4.39 | 3 | 3.70 | 0.81 | 3 |
| 2268 | AAGTACACCAGGAACGAGGCCAGGA | −9.39 | 4.33 | 3 | −10.32 | 1.90 | 3 | 6.59 | 3.65 | 3 | 5.59 | 6.88 | 3 |
| 3637 | AGTACACCAGGAACGAGGCCAGG | −13.72 | 4.46 | 3 | −6.94 | 6.67 | 3 | −1.11 | 11.33 | 3 | 5.86 | 9.58 | 3 |
| 4521 | AGTACACSAGGAACGSGGCCAGG | −10.97 | 2.72 | 3 | −3.35 | 3.85 | 3 | −1.89 | 5.81 | 3 | 4.51 | 4.02 | 3 |
| 3638 | AAGTACACCAGGAACGAGGCCAG | −11.25 | 2.10 | 3 | −8.14 | 7.24 | 3 | 3.18 | 3.03 | 3 | −0.08 | 8.78 | 3 |
| 4520 | AAGTACASCAGGAACSAGGCCAG | −9.94 | 4.34 | 3 | 3.20 | 3.53 | 3 | −2.53 | 4.63 | 3 | 11.09 | 2.93 | 3 |
| 3636 | GTACACCAGGAACGAGGCCAGGA | −2.32 | 0.94 | 3 | −15.34 | 0.39 | 3 | 16.61 | 1.16 | 3 | −6.81 | 4.93 | 3 |
| 4519 | GTACACCSGGAACGASGCCAGGA | −11.44 | 3.41 | 3 | −12.13 | 1.03 | 3 | −1.60 | 7.70 | 3 | −4.08 | 2.37 | 3 |
| 2283 | TCCCCCTTCTCTGCCAAGTACACCA | −8.58 | 0.52 | 3 | 1.38 | 2.41 | 3 | −1.96 | 1.59 | 3 | 8.98 | 2.74 | 3 |
| 2298 | TCAAAGTGGTCGTTCTCCCCCTTCT | −22.23 | 0.80 | 3 | −8.71 | 3.31 | 3 | −20.40 | 1.04 | 3 | −7.81 | 3.68 | 3 |
| 3667 | CAAAGTGGTCGTTCTCCCCCTTC | −15.09 | 0.64 | 3 | −12.54 | 0.65 | 3 | −15.86 | 0.43 | 3 | −9.26 | 1.70 | 3 |
| 4524 | CAAAGTGSTCGTTCTSCCCCTTC | −19.11 | 4.04 | 3 | −26.45 | 1.15 | 3 | −15.49 | 5.45 | 3 | −26.40 | 2.00 | 3 |
| 3668 | TCAAAGTGGTCGTTCTCCCCCTT | −9.51 | 1.09 | 3 | −14.08 | 2.43 | 3 | −8.55 | 0.42 | 3 | −13.79 | 3.70 | 3 |
| 4523 | TCAAAGTSGTCGTTCSCCCCCTT | −17.92 | 2.25 | 3 | −11.73 | 0.79 | 3 | −16.53 | 0.91 | 3 | −9.02 | 2.70 | 3 |
| 3666 | AAAGTGGTCGTTCTCCCCCTTCT | −15.05 | 1.82 | 3 | −11.97 | 2.72 | 3 | −11.83 | 1.19 | 3 | −7.04 | 4.94 | 3 |
| 4522 | AAAGTGGSCGTTCTCSCCCTTCT | −17.53 | 2.49 | 3 | −7.72 | 2.74 | 3 | −14.23 | 2.57 | 3 | −7.18 | 3.43 | 3 |
| 2307 | GCGTAGGTGTCAAAGTGGTCGTTCT | −12.37 | 0.72 | 3 | −0.59 | 0.24 | 3 | −17.33 | 1.02 | 3 | −5.25 | 0.38 | 3 |
| 2322 | CACCAGAGTGCATCCGCGTAGGTGT | −7.17 | 1.07 | 3 | −2.02 | 3.43 | 3 | −8.21 | 2.19 | 3 | −2.94 | 1.99 | 3 |
| 2342 | GACCACAACTCACCAGGCCCCACCA | −3.52 | 0.15 | 3 | 3.98 | 2.84 | 3 | 2.06 | 0.84 | 3 | 11.28 | 3.63 | 3 |
| 2385 | ACAGGGACAGGGGTGTATCAGCAGG | −2.02 | 1.69 | 3 | −6.47 | 0.93 | 3 | 2.29 | 1.79 | 3 | −3.01 | 0.99 | 3 |
| 3754 | CAGGGACAGGGGTGTATCAGCAG | −1.46 | 3.58 | 3 | −7.13 | 1.45 | 3 | −2.39 | 1.23 | 3 | −2.78 | 1.35 | 3 |
| 4527 | CAGGGACSGGGGTGTSTCAGCAG | −4.18 | 2.57 | 3 | −8.72 | 0.65 | 3 | 0.94 | 5.79 | 3 | −5.29 | 1.58 | 3 |
| 3755 | ACAGGGACAGGGGTGTATCAGCA | −3.74 | 0.92 | 3 | −10.27 | 0.71 | 3 | −0.91 | 2.00 | 3 | −7.14 | 2.05 | 3 |
| 4526 | ACAGGGASAGGGGTGSATCAGCA | −11.18 | 0.55 | 3 | 0.67 | 0.61 | 3 | −7.16 | 0.42 | 3 | 5.91 | 2.03 | 3 |
| 3753 | AGGGACAGGGGTGTATCAGCAGG | −7.05 | 2.23 | 3 | −4.52 | 3.05 | 3 | −1.24 | 1.16 | 3 | −2.67 | 3.84 | 3 |
| 4525 | AGGGACASGGGTGTASCAGCAGG | −0.83 | 2.45 | 3 | −8.90 | 0.60 | 3 | 1.58 | 2.52 | 3 | −5.58 | 1.62 | 3 |
| 2422 | ATGGAGCAGGCTCAGCCAGTGAGAG | 27.64 | 11.01 | 3 | 22.46 | 4.41 | 3 | 34.84 | 9.98 | 3 | 31.70 | 4.70 | 3 |
| 2434 | CCAAAGAGATGTATGGAGCAGGCTC | 25.65 | 6.66 | 3 | 27.85 | 8.30 | 3 | 38.45 | 12.30 | 3 | 34.42 | 10.27 | 3 |
| 2446 | CAGCAGGTGGCCCCAAAGAGATGTA | 14.58 | 11.65 | 3 | 31.12 | 5.29 | 3 | 18.13 | 16.07 | 3 | 41.88 | 7.13 | 3 |
| 2465 | CATCATGACCACGGGCAGCCAGCAG | 41.69 | 3.91 | 3 | 28.44 | 2.21 | 3 | 51.84 | 4.79 | 3 | 38.90 | 4.44 | 3 |
| 2477 | TCGACCACAAGCCATCATGACCACG | 11.77 | 0.06 | 3 | 8.75 | 2.23 | 3 | 23.04 | 1.41 | 3 | 15.12 | 3.46 | 3 |
| 3846 | CGACCACAAGCCATCATGACCAC | −9.69 | 2.12 | 3 | 7.70 | 3.00 | 3 | −2.77 | 2.51 | 3 | 12.13 | 4.02 | 3 |
| 4530 | CGACCACSAGCCATCSTGACCAC | 3.86 | 2.82 | 3 | −0.56 | 2.08 | 3 | 7.82 | 2.26 | 3 | −0.74 | 2.31 | 3 |
| 3847 | TCGACCACAAGCCATCATGACCA | −7.18 | 2.11 | 3 | −10.87 | 5.29 | 3 | −2.44 | 1.28 | 3 | −6.74 | 3.63 | 3 |
| 4529 | TCGACCASAAGCCATSATGACCA | −8.50 | 2.66 | 3 | 1.19 | 6.06 | 3 | −6.71 | 2.36 | 3 | 4.36 | 7.98 | 3 |
| 3845 | GACCACAAGCCATCATGACCACG | −5.69 | 0.39 | 3 | 7.08 | 3.00 | 3 | 0.12 | 1.32 | 3 | 9.58 | 5.60 | 3 |
| 4528 | GACCACASGCCATCASGACCACG | 2.37 | 1.29 | 3 | −3.03 | 3.45 | 3 | 10.91 | 2.03 | 3 | 0.54 | 2.45 | 3 |
| 2492 | AACCACCACCCCGCCTCGACCACAA | 34.71 | 1.02 | 3 | 32.62 | 4.60 | 3 | 78.59 | 11.49 | 3 | 39.00 | 5.11 | 3 |
| 2121 | TCAACACCATGACCACCATCACCAC | 2.82 | 3.79 | 3 | 4.06 | 1.82 | 3 | 13.25 | 1.50 | 3 | 8.35 | 2.18 | 3 |
| 3490 | CAACACCATGACCACCATCACCA | −23.86 | 3.03 | 3 | −4.90 | 0.75 | 3 | 1.72 | 2.65 | 3 | 3.76 | 2.55 | 3 |
| 4509 | CAACACCSTGACCACSATCACCA | −0.86 | 2.02 | 3 | −7.48 | 1.73 | 3 | 7.51 | 3.61 | 3 | −4.29 | 2.55 | 3 |
| 3491 | TCAACACCATGACCACCATCACC | 4.79 | 3.15 | 3 | −0.23 | 0.59 | 3 | 15.16 | 0.91 | 3 | 4.09 | 2.15 | 3 |
| 4508 | TCAACACSATGACCASCATCACC | −10.85 | 1.92 | 3 | −10.52 | 3.21 | 3 | −6.61 | 0.58 | 3 | −4.52 | 1.51 | 3 |
| 3489 | AACACCATGACCACCATCACCAC | 0.11 | 1.01 | 3 | −5.62 | 0.66 | 3 | 15.37 | 2.99 | 3 | −1.85 | 2.14 | 3 |
| 4507 | AACACCASGACCACCSTCACCAC | −8.42 | 1.65 | 3 | −5.16 | 1.62 | 3 | −0.51 | 2.49 | 3 | −2.96 | 0.99 | 3 |
| 1780 | AGCCACCACTGACTCCATCCCTCCA | 18.29 | 7.59 | 3 | 34.42 | 3.34 | 3 | 42.41 | 7.78 | 3 | 50.15 | 7.27 | 3 |
| 1783 | CAGAGCCACCACTGACTCCATCCCT | −0.42 | 2.35 | 3 | 1.83 | 2.82 | 3 | 1.16 | 3.66 | 3 | 7.25 | 0.76 | 3 |
| 3152 | AGAGCCACCACTGACTCCATCCC | −1.67 | 7.55 | 3 | 8.17 | 1.95 | 3 | −0.58 | 10.45 | 3 | 13.50 | 3.41 | 3 |
| 4485 | AGAGCCASCACTGACSCCATCCC | −1.74 | 1.39 | 3 | −2.22 | 1.69 | 3 | 6.08 | 2.73 | 3 | 6.46 | 1.34 | 3 |
| 3153 | CAGAGCCACCACTGACTCCATCC | −7.13 | 0.21 | 3 | −1.63 | 1.38 | 3 | −2.53 | 4.15 | 3 | 3.53 | 1.74 | 3 |
| 4484 | CAGAGCCSCCACTGASTCCATCC | −5.40 | 1.19 | 3 | 3.99 | 1.97 | 3 | 1.32 | 0.98 | 3 | 7.07 | 2.50 | 3 |
| 3151 | GAGCCACCACTGACTCCATCCCT | −2.85 | 8.97 | 3 | −1.39 | 1.80 | 3 | 5.54 | 9.87 | 3 | 5.03 | 2.03 | 3 |
| 4483 | GAGCCACSACTGACTSCATCCCT | −12.63 | 1.26 | 3 | 0.34 | 4.74 | 3 | −10.50 | 3.13 | 3 | 4.90 | 4.65 | 3 |
| 1786 | AGCCAGAGCCACCACTGACTCCATC | −1.89 | 6.71 | 3 | 4.39 | 5.55 | 3 | 5.42 | 9.34 | 3 | 14.40 | 2.83 | 3 |
| 1789 | TCCAGCCAGAGCCACCACTGACTCC | −1.21 | 4.35 | 3 | 1.54 | 5.17 | 3 | 14.28 | 13.50 | 3 | 8.32 | 6.95 | 3 |
| 1809 | CTCGTTCACACCTGATGGGCTCCAG | −10.05 | 5.15 | 3 | 4.85 | 1.13 | 3 | −7.21 | 3.62 | 3 | 9.13 | 3.75 | 3 |
| 3178 | TCGTTCACACCTGATGGGCTCCA | −7.80 | 1.65 | 3 | −1.68 | 2.26 | 3 | −5.88 | 0.42 | 3 | 3.80 | 2.86 | 3 |
| 4488 | TCGTTCASACCTGATSGGCTCCA | 1.17 | 0.29 | 3 | −5.21 | 4.34 | 3 | 3.95 | 2.65 | 3 | −4.67 | 2.61 | 3 |
| 3179 | CTCGTTCACACCTGATGGGCTCC | −6.62 | 5.41 | 3 | −4.12 | 2.77 | 3 | −1.32 | 1.64 | 3 | −3.48 | 2.17 | 3 |
| 4487 | CTCGTTCSCACCTGASGGGCTCC | −9.29 | 0.93 | 3 | −18.77 | 5.14 | 3 | −6.68 | 1.03 | 3 | −17.26 | 2.99 | 3 |
| 3177 | CGTTCACACCTGATGGGCTCCAG | −22.64 | 2.20 | 3 | −26.65 | 3.28 | 3 | −21.21 | 1.30 | 3 | −21.96 | 0.95 | 3 |
| 4486 | CGTTCACSCCTGATGSGCTCCAG | 30.72 | 3.39 | 3 | −2.00 | 3.97 | 3 | 23.60 | 4.03 | 3 | −7.70 | 2.43 | 3 |
| 4470 | CTCGTTSACACCTGATGGSCTCCAG | 3.31 | 5.98 | 3 | −8.32 | 4.06 | 3 | 15.59 | 11.34 | 3 | −0.88 | 0.58 | 3 |
| 4474 | CTCGTTCASACCTGATGGSCTCCAG | 13.25 | 12.40 | 3 | 15.47 | 4.14 | 3 | 20.50 | 10.05 | 3 | 32.33 | 5.74 | 3 |
| 1812 | AGGCTCGTTCACACCTGATGGGCTC | 11.70 | 4.10 | 3 | 11.57 | 2.92 | 3 | 21.34 | 3.17 | 3 | 28.03 | 2.49 | 3 |
| 4496 | ACCAGASGCATCACCATCSCCACCA | 3.64 | 1.09 | 3 | 22.71 | 3.04 | 3 | 39.79 | 2.17 | 3 | 53.79 | 54.11 | 3 |
| 4502 | ACCAGAAGSATCACCATCSCCACCA | 9.40 | 2.42 | 3 | 10.68 | 8.44 | 3 | 18.97 | 7.44 | 3 | 16.09 | 6.87 | 3 |
| 1815 | GGGAGGCTCGTTCACACCTGATGGG | 16.74 | 6.47 | 3 | 5.72 | 1.11 | 3 | 21.02 | 2.36 | 3 | 10.87 | 2.62 | 3 |
| 1818 | GGAGGGAGGCTCGTTCACACCTGAT | 4.34 | 1.25 | 3 | 3.98 | 2.02 | 3 | 2.42 | 2.83 | 3 | 4.42 | 1.85 | 3 |
| 3187 | GAGGGAGGCTCGTTCACACCTGA | −2.33 | 2.32 | 3 | −7.92 | 1.78 | 3 | 5.16 | 5.38 | 3 | −8.91 | 1.16 | 3 |
| 4491 | GAGGGAGSCTCGTTCSCACCTGA | −0.84 | 5.15 | 3 | −9.91 | 3.20 | 3 | 0.25 | 6.70 | 3 | −5.57 | 3.10 | 3 |
| 3188 | GGAGGGAGGCTCGTTCACACCTG | 2.64 | 2.00 | 3 | −4.17 | 2.78 | 3 | −0.15 | 0.84 | 3 | −2.47 | 5.32 | 3 |
| 4490 | GGAGGGASGCTCGTTSACACCTG | −5.69 | 0.85 | 3 | −0.56 | 3.57 | 3 | −2.92 | 1.41 | 3 | −1.30 | 2.34 | 3 |
| 3186 | AGGGAGGCTCGTTCACACCTGAT | −3.48 | 2.89 | 3 | −5.92 | 1.08 | 3 | −3.05 | 3.85 | 3 | −3.55 | 4.75 | 3 |
| 4489 | AGGGAGGSTCGTTCASACCTGAT | −10.48 | 3.10 | 3 | −16.43 | 1.36 | 3 | −14.43 | 2.77 | 3 | −15.99 | 3.76 | 3 |
| 1843 | CGAGAAGAAGCACCGAGAAGAAAGG | 1.88 | 1.81 | 3 | 20.60 | 4.70 | 3 | 5.24 | 9.03 | 3 | 33.13 | 5.11 | 3 |
| 1848 | AGTCACGAGAAGAAGCACCGAGAAG | 20.23 | 4.29 | 3 | 7.44 | 4.76 | 3 | 18.02 | 3.26 | 3 | 6.67 | 1.30 | 3 |
| 3217 | GTCACGAGAAGAAGCACCGAGAA | 13.12 | 0.72 | 3 | 9.52 | 5.98 | 3 | 10.08 | 0.51 | 3 | 9.58 | 3.28 | 3 |
| 4494 | GTCACGASAAGAAGCSCCGAGAA | 9.68 | 2.36 | 3 | −0.91 | 6.39 | 3 | 10.12 | 1.61 | 3 | −1.73 | 7.56 | 3 |
| 3218 | AGTCACGAGAAGAAGCACCGAGA | 16.86 | 1.93 | 3 | 8.85 | 0.49 | 3 | 14.17 | 4.84 | 3 | 5.65 | 1.53 | 3 |
| 4493 | AGTCACGSGAAGAAGSACCGAGA | −3.49 | 5.85 | 3 | 14.86 | 1.55 | 3 | −6.76 | 7.53 | 3 | 14.20 | 2.40 | 3 |
| 3216 | TCACGAGAAGAAGCACCGAGAAG | 3.79 | 1.42 | 3 | 0.36 | 1.80 | 3 | 2.52 | 1.86 | 3 | −2.15 | 2.36 | 3 |
| 4492 | TCACGAGSAGAAGCASCGAGAAG | 11.30 | 3.09 | 3 | 0.00 | 0.89 | 3 | 13.30 | 1.65 | 3 | 0.10 | 3.57 | 3 |
| 4471 | AGTCACSAGAAGAAGCACSGAGAAG | −7.58 | 4.89 | 3 | −8.35 | 9.54 | 3 | 4.67 | 6.75 | 3 | 0.76 | 11.81 | 3 |
| 4475 | AGTCACGASAAGAAGCACSGAGAAG | 9.02 | 10.30 | 3 | −17.71 | 8.26 | 3 | 29.48 | 10.29 | 3 | −9.94 | 10.72 | 3 |
| 4498 | AAGTACSCCAGGAACGAGSCCAGGA | −1.31 | 3.42 | 3 | 5.34 | 2.71 | 3 | −2.48 | 2.14 | 3 | 4.78 | 3.59 | 3 |
| 4504 | AAGTACACSAGGAACGAGSCCAGGA | −1.30 | 1.10 | 3 | 2.26 | 1.78 | 3 | −0.81 | 2.06 | 3 | 1.16 | 2.09 | 3 |
| 1749 | GCTCTTGAAGCAAACCCCAGGCAGG | −3.34 | 2.72 | 3 | −10.58 | 0.70 | 3 | −2.62 | 1.30 | 3 | −10.08 | 2.61 | 3 |
| 3118 | CTCTTGAAGCAAACCCCAGGCAG | −11.90 | 0.52 | 3 | −11.40 | 0.41 | 3 | −13.21 | 1.98 | 3 | −11.64 | 1.20 | 3 |
| 4479 | CTCTTGASGCAAACCSCAGGCAG | −7.68 | 3.19 | 3 | −9.11 | 2.70 | 3 | −6.38 | 3.39 | 3 | −7.82 | 3.19 | 3 |
| 3119 | GCTCTTGAAGCAAACCCCAGGCA | −4.09 | 1.67 | 3 | −4.91 | 2.51 | 3 | 0.26 | 1.95 | 3 | −2.87 | 2.17 | 3 |
| 4478 | GCTCTTGSAGCAAACSCCAGGCA | −3.12 | 0.54 | 3 | −1.01 | 6.66 | 3 | −0.87 | 4.06 | 3 | 4.96 | 7.18 | 3 |
| 3117 | TCTTGAAGCAAACCCCAGGCAGG | −10.82 | 0.13 | 3 | −9.64 | 0.82 | 3 | −8.40 | 1.21 | 3 | −9.49 | 1.64 | 3 |
| 4477 | TCTTGAASCAAACCCSAGGCAGG | −5.69 | 1.71 | 3 | −5.51 | 4.26 | 3 | −2.67 | 2.32 | 3 | −2.79 | 4.41 | 3 |
| 1774 | CACTGACTCCATCCCTCCACCCAGG | 3.71 | 0.79 | 3 | 1.85 | 2.54 | 3 | 2.85 | 1.65 | 3 | 1.59 | 2.04 | 3 |
| 3143 | ACTGACTCCATCCCTCCACCCAG | 5.30 | 5.18 | 3 | 2.26 | 1.06 | 3 | 2.74 | 1.78 | 3 | 0.04 | 2.37 | 3 |
| 4482 | ACTGACTSCATCCCTSCACCCAG | 1.00 | 2.04 | 3 | −1.50 | 1.79 | 3 | 2.36 | 2.04 | 3 | −2.04 | 2.02 | 3 |
| 3144 | CACTGACTCCATCCCTCCACCCA | 0.91 | 1.61 | 3 | −0.70 | 1.58 | 3 | 1.60 | 1.21 | 3 | −1.03 | 0.32 | 3 |
| 4481 | CACTGACSCCATCCCSCCACCCA | 4.83 | 1.31 | 3 | 1.79 | 0.97 | 3 | 3.25 | 1.75 | 3 | 1.13 | 2.26 | 3 |
| 3142 | CTGACTCCATCCCTCCACCCAGG | 6.92 | 1.97 | 3 | 6.66 | 2.08 | 3 | 8.17 | 1.78 | 3 | 8.13 | 3.21 | 3 |
| 4480 | CTGACTCSATCCCTCSACCCAGG | 13.39 | 2.34 | 3 | 2.54 | 6.61 | 3 | 11.08 | 0.98 | 3 | 3.64 | 6.11 | 3 |
| 4499 | ACAGGGSCAGGGGTGTATSAGCAGG | 6.59 | 5.05 | 3 | 6.33 | 1.86 | 3 | 14.95 | 6.32 | 3 | 13.10 | 1.60 | 3 |
| 4505 | ACAGGGACSGGGGTGTATSAGCAGG | 7.47 | 0.68 | 3 | 7.48 | 1.97 | 3 | 11.20 | 0.98 | 3 | 13.21 | 3.65 | 3 |
| 4500 | TCGACCSCAAGCCATCATSACCACG | 18.23 | 5.32 | 3 | 14.37 | 5.75 | 3 | 27.23 | 4.59 | 3 | 21.77 | 2.94 | 3 |
| 4506 | TCGACCACSAGCCATCATSACCACG | 34.70 | 9.05 | 3 | 31.42 | 5.95 | 3 | 60.23 | 10.63 | 3 | 43.91 | 8.79 | 3 |
| 4472 | GCTCTTSAAGCAAACCCCSGGCAGG | 6.15 | 7.74 | 3 | −0.54 | 0.82 | 3 | 14.16 | 10.84 | 3 | 9.32 | 4.10 | 3 |
| 4476 | GCTCTTGASGCAAACCCCSGGCAGG | 2.45 | 2.87 | 3 | 14.93 | 6.65 | 3 | 10.31 | 3.46 | 3 | 18.48 | 0.39 | 3 |
| 4495 | TCAACASCATGACCACCASCACCAC | 19.19 | 5.02 | 3 | 13.50 | 2.06 | 3 | 20.16 | 4.33 | 3 | 13.88 | 4.01 | 3 |
| 4501 | TCAACACCSTGACCACCASCACCAC | 5.52 | 5.25 | 3 | 18.65 | 3.28 | 3 | 18.45 | 9.19 | 3 | 22.97 | 5.99 | 3 |

*Unless otherwise noted, each of the nucleosides of antisense oligonucleotides shown in Table 34 are modified nucleosides with 2′-O-(2-methoxyethyl) (2′-MOE) sugar moieties, each “C” is replaced with a 5-methylcytosine (5-MeC), and all internucleoside linkages are phosphorothioate linkages. A spacer as indicated by S is not a nucleoside. S represents a spacer of Formula (Iia′) as disclosed herein.

### Example 10: Design and Selection of UNC13A Oligonucleotides

UNC13A AONs oligonucleotides that target a UNC13A transcript are designed and tested to identify UNC13A AONs capable of reducing quantity of UNC13A transcripts (e.g., mis-spliced UNC13A transcripts). Such UNC13A AONs include UNC13A parent oligonucleotides represented by any of SEQ ID NOs: 4531-5794 or UNC13A oligonucleotide variants represented by SEQ ID NOs: 7059-8322. The UNC13A parent oligonucleotides are 25 nucleosides in length. Each of the nucleosides of the UNC13A parent oligonucleotides are modified nucleosides with 2′-MOE sugar moieties, and each “C” is replaced with a 5-MeC. Additionally, each of the internucleoside linkages between the nucleosides of the UNC13A oligonucleotides are phosphorothioate internucleoside linkages.

Generally, the length of the UNC13A antisense oligonucleotides are 25 oligonucleotide units in length. However, variants of the UNC13A antisense oligonucleotides were also designed with varying lengths (e.g., 23mers, 21mers, or 19mers). Examples of these variant UNC13A antisense oligonucleotides were designed to include a subset of the sequences of SEQ ID NOs: 7059-8322.

TABLE 35A
Example UNC13A AONs (including UNC13A oligonucleotides with
two spacers)

| | Oligonucleotide Sequence* | |
|---|---|---|
| SEQ ID | (where S indicates presence | |
| No: | of a Spacer) (5′→3′) | Length |
| 9663 | GGGCAGSCAGGAATGGTGSGTGGAA | 25 |
| 9664 | AGGGAGSAGTTTTCCAGGSAAAGGG | 25 |
| 9665 | GCAGCTSGAAGAGACATASCCAGAC | 25 |
| 9666 | TCCACCSGCCACTCACAASCATCCA | 25 |
| 9667 | GAAAGTSTCATGGAGAGTSCAAGGG | 25 |
| 9668 | GGGCAGGCSGGAATGGTGSGTGGAA | 25 |
| 9669 | AGGGAGGASTTTTCCAGGSAAAGGG | 25 |
| 9670 | GCAGCTGGSAGAGACATASCCAGAC | 25 |
| 9671 | TCCACCAGSCACTCACAASCATCCA | 25 |
| 9672 | GAAAGTGTSATGGAGAGTSCAAGGG | 25 |
| 9673 | GCAGGCASGAATGGTSAGTGGAA | 23 |
| 9674 | GGGCAGGSAGGAATGSTGAGTGG | 23 |
| 9675 | GGCAGGCSGGAATGGSGAGTGGA | 23 |
| 9676 | GTAAAGGSAATGGCASGGCGGGC | 23 |
| 9677 | AGGTAAASGGAATGGSATGGCGG | 23 |
| 9678 | GGTAAAGSGAATGGCSTGGCGGG | 23 |
| 9679 | AGGAGGTSACCCTGASTCTGGAC | 23 |
| 9680 | AGAGGAGSTGACCCTSAATCTGG | 23 |
| 9681 | GAGGAGGSGACCCTGSATCTGGA | 23 |
| 9682 | ACAAACGSCCCAATCSTGAGTGG | 23 |
| 9683 | ACACAAASGGCCCAASCCTGAGT | 23 |
| 9684 | CACAAACSGCCCAATSCTGAGTG | 23 |
| 9685 | TCTTTCCSGGAAACCSAGGCAGC | 23 |
| 9686 | GTTCTTTSCAGGAAASCCAGGCA | 23 |
| 9687 | TTCTTTCSAGGAAACSCAGGCAG | 23 |
| 9688 | TCCGCAASTTAATCCSCCTACCC | 23 |
| 9689 | GATCCGCSACTTAATSCACCTAC | 23 |
| 9690 | ATCCGCASCTTAATCSACCTACC | 23 |
| 9691 | CCCACCCSTCTAACTSCCCCAAA | 23 |
| 9692 | CACCCACSCATCTAASTACCCCA | 23 |
| 9693 | ACCCACCSATCTAACSACCCCAA | 23 |
| 9694 | AAGTGTCSTGGAGAGSGCAAGGG | 23 |
| 9695 | GAAAGTGSCATGGAGSGTGCAAG | 23 |
| 9696 | AAAGTGTSATGGAGASTGCAAGG | 23 |

*Unless otherwise noted, each of the nucleosides of antisense oligonucleotides shown in Table 35A are modified nucleosides with 2′-O-(2-methoxyethyl) (2′-MOE) sugar moieties, each “C” is replaced with a 5-methylcytosine (5-MeC), and all internucleoside linkages are phosphorothioate linkages. A spacer as indicated by S is not a nucleoside. S represents a spacer of Formula (Iia′) as disclosed herein.

Methods for Synthesizing a Spacer building block for Spacers of Formula (IIib′)

Several UNC13A AONs were synthesized with one or more spacers of Formula (IIib′), as disclosed herein. The method of generating the spacer building block involved a 5 step synthesis method, described below.

### Step 1: Benzoyl Protection

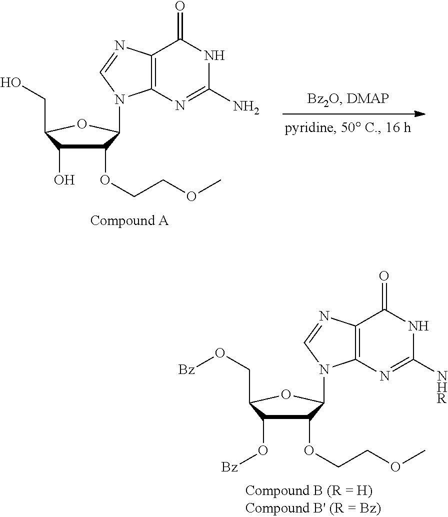

### (2R,3R,4R,5R)-5-(2-amino-6-oxo-1,6-dihydro-9H-purin-9-yl)-2-((benzoyloxy)methyl)-4-(2-methoxyethoxy)tetrahydrofuran-3-yl benzoate (2) & (2R,3R,4R,5R)-5-(2-benzamido-6-oxo-1,6-dihydro-9H-purin-9-yl)-2-((benzoyloxy)methyl)-4-(2-methoxyethoxy) tetrahydrofuran-3-yl benzoate (2′)

To a solution of 2′-O-(2-methoxyethyl)-guanosine (15.00 g, 42.6 mmol) in dry pyridine (300 mL was added benzoic anhydride (26.2 g, 114 mmol) followed by 4-dimethylaminopyridine (1.77 g, 14.2 mmol) at rt, then the temperature was raised to 50° C. and allowed to stir for 16 h. LCMS showed complete consumption of starting material. The reaction was then diluted with water, quenched with saturated aqueous sodium bicarbonate and extracted with ethyl acetate (×3). The organic layers were further washed with brine, then dried over sodium sulfate and concentrated in vacuo. The crude product was purified by silica gel column chromatography (0-15% methanol in dichloromethane) to give an approximate 2:1 mixture of compound B and compound B′ (20.0 g) as a colorless oil. Calculated mass for B: C₂₇H₂₇N₅O₈=549.19. found=550.3 (M+H)⁺. Calculated mass for B′: C₃₄H₃₁N₅O₉=653.21. found 654.3 (M+H)⁺. LCMS: CSH C18 AMB (3 min): compound B, R_{T}=1.33 min, compound B′, R_{T}=1.48 min.

### Step 2: Reductive Nucleobase Cleavage

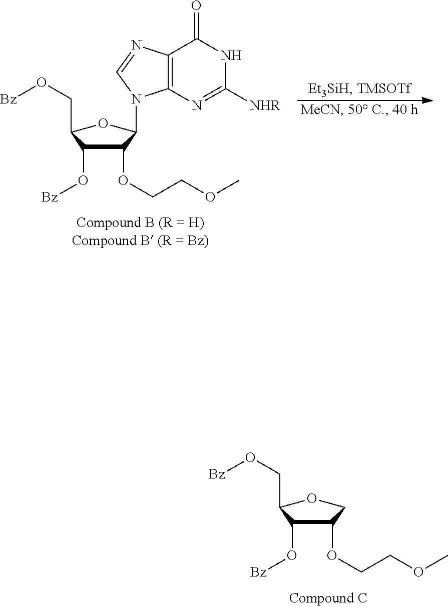

### ((2R,3S,4S)-3-(benzoyloxy)-4-(2-methoxyethoxy) tetrahydrofuran-2-yl) methyl benzoate (3)

To a mixture of compound B & B′ (20.0 g) in acetonitrile (120 mL) was added triethylsilane (49.3 mL, 306 mmol) followed by trimethylsilyl trifluoromethanesulfonate (37.3 mL, 204 mmol) dropwise at rt then the temperature was raised to 50° C. and allowed to stir for 16 h. Additional triethylsilane (28.9 mL, 179 mmol) and trimethylsilyl trifluoromethanesulfonate (21.8 mL, 119 mmol) was added and the reaction was continued at 50° C. for an additional 24 h. LCMS showed complete consumption of starting material, therefore the reaction was quenched with saturated aqueous sodium bicarbonate then extracted with diethyl ether (×3). The organic layers were combined and further washed with brine, then dried over sodium sulfate and concentrated in vacuo. The crude product was purified by silica gel column chromatography (0-50% ethyl acetate in hexanes) to give compound C (5.61 g, 33% yield over 2 steps) as a colorless oil. Calculated mass for C₂₂H₂₄O₇=400.15. found=401.3 (M+H)⁺.

¹H NMR (400 MHz, CDCl₃): δ 8.13-7.99 (m, 4H), 7.63-7.50 (m, 2H), 7.49-7.36 (m, 4H), 5.40-5.33 (m, 1H), 4.63-4.53 (m, 1H), 4.52-4.42 (m, 2H), 4.34 (q, J=5.6 Hz, 1H), 4.24 (dd, J=9.4, 5.7 Hz, 1H), 3.96 (dd, J=9.4, 5.7 Hz, 1H), 3.69-3.63 (m, 2H), 3.45-3.39 (m, 2H), 3.23 (s, 3H). LCMS: CSH C18 AMB (3 min): R_{T}=1.55 min

### Step 3: Saponification

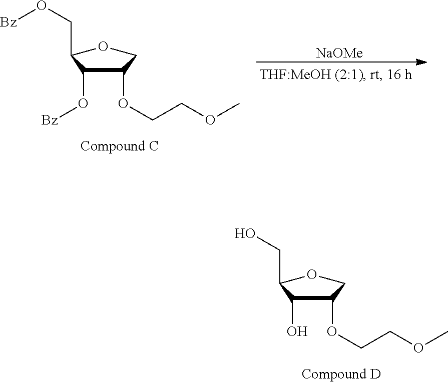

### (2R,3R,4S)-2-(hydroxymethyl)-4-(2-methoxyethoxy) tetrahydrofuran-3-ol (4)

To a solution of compound C (5.00 g, 12.5 mmol) in tetrahydrofuran (60 mL) and methanol (30 mL) was added a 25% solution of sodium methoxide in methanol (14.3 mL, 214 mmol) at rt and the reaction mixture was stirred at rt for 16 h. LCMS and TLC showed complete consumption of staring material. The reaction mixture was then concentrated in vacuo and purified by silica gel column chromatography (0-10% methanol in dichloromethane) to give compound D (1.50 g, 62% yield) as a colorless oil.

¹H NMR (400 MHz, CDCl₃): δ 4.08-4.00 (m, 2H), 3.94 (dd, J=10.0, 5.1 Hz, 1H), 3.82-3.74 (m, 4H), 3.69-3.45 (m, 4H), 3.36 (s, 3H), 3.32 (s, 2H).

¹³C NMR (101 MHz, CDCl₃): δ 83.84, 79.60, 71.93, 71.44, 70.67, 69.87, 62.56, 59.07.

### Step 4: DMT-Protection

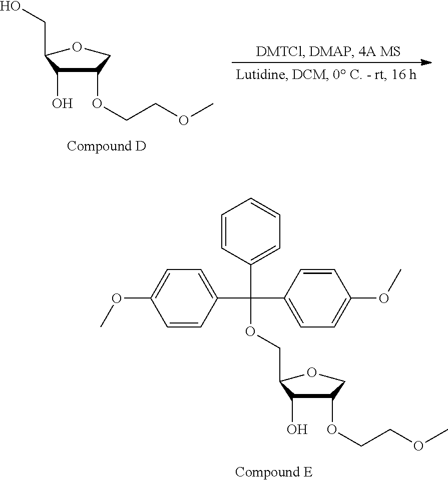

### (2R,3S,4S)-2-((bis(4-methoxyphenyl) (phenyl)methoxy) methyl)-4-(2-hydroxyethoxy) tetrahydrofuran-3-ol (5)

To a suspension of compound D (1.00 g, 5.20 mmol) and 4 Å molecular sieves (120 mg) in 2,6-lutidine (20.0 mL) was added 4-dimethylaminopyridine (128 mg, 1.04 mmol) followed by a solution of 4,4′-dimethoxytrityl chloride (3.60 g, 10.4 mmol) in dichloromethane (10.0 mL) at 0° C., then slowly raised the temperature to rt and allowed to stir for 16 h. LCMS and TLC showed the reaction was complete, therefore hexanes (10 mL) and methanol (10 mL) were added and the mixture was concentrated in vacuo. The residue was then dissolved in ethyl acetate and washed with saturated aqueous copper sulfate (×2) followed by brine, then dried over sodium sulfate and concentrated in vacuo. The crude product was purified by silica gel column chromatography (0-50% ethyl acetate in hexanes) to give compound E (1.85 g, 72% yield) as a pale-yellow oil. Calculated mass for C₂₉H₃₄O₇=494.23. found 493.5 (M−H)⁻.

¹H NMR (400 MHz, CDCl₃): δ 7.47-7.40 (m, 2H), 7.37-7.30 (m, 3H), 7.30-7.22 (m, 3H), 7.21-7.13 (m, 1H), 6.81 (dd, J=8.8, 1.7 Hz, 4H), 4.17-4.11 (m, 1H), 4.11-4.03 (m, 2H), 4.00-3.93 (m, 1H), 3.86-3.79 (m, 2H), 3.78-3.74 (m, 6H), 3.70-3.62 (m, 1H), 3.61-3.47 (m, 2H), 3.40-3.36 (m, 3H), 3.34-3.27 (m, 2H), 3.12-3.07 (m, 1H).

LCMS: CSH C18 AMB (7 min): R_{T}=3.28 min

### Step 5: Phosphoramidite Installation

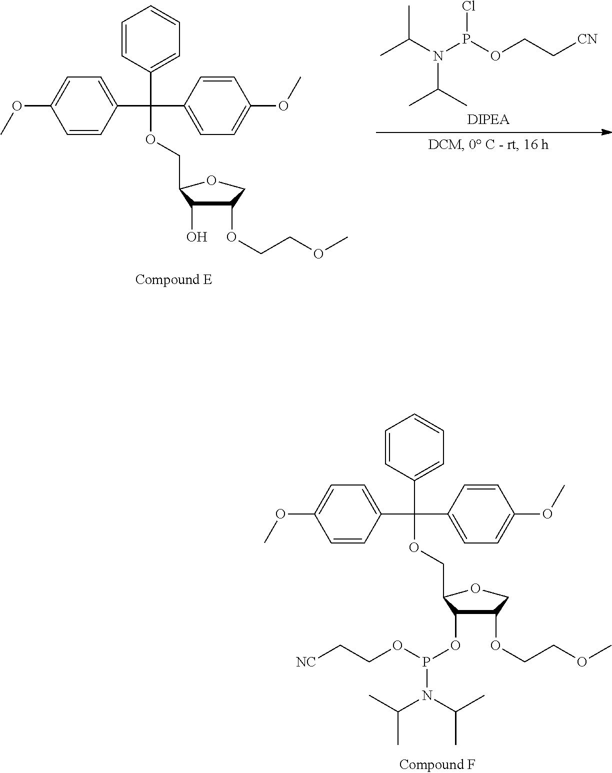

### (2R,3S,4S)-2-((bis(4-methoxyphenyl) (phenyl)methoxy) methyl)-4-(2-methoxyethoxy) tetrahydrofuran-3-yl (2-cyanoethyl) diisopropylphosphoramidite

To a suspension of compound E (6.85 g, 13.9 mmol) in dry dichloromethane (150 mL) was added N,N-diisopropylethylamine (21.1 mL, 120 mmol) and the resulting solution was cooled to 0° C. under a nitrogen atmosphere. 2-Cyanoethyl N,N-diisopropylchlorophosphoramidite (6.18 mL, 27.7 mmol) was then added dropwise and the resulting solution was allowed to attain rt and stir for 16 h. LCMS and TLC showed the reaction was complete. The reaction mixture was then cooled to 0° C., diluted with dichloromethane and washed with cold saturated aqueous sodium bicarbonate, followed by brine, then dried over sodium sulfate and concentrated in vacuo. The crude product was then purified by silica gel column chromatography (5% triethylamine in ethyl acetate and hexanes, 5-80%) to give, after lyophilization, Compound F (6.35 g, 66% yield) as a colorless oil (mixture of diastereomers at P). Calculated mass for C₃₈H₅₁N₂O₈P=694.34. found=695.6 (M+H)⁺.

### Example 11: Methods for Evaluating UNC13a Antisense Oligonucleotides

UNC13A antisense oligonucleotides were evaluated in iPSC derived human motor neurons (hMN). The cells were seeded in 96-well plates at a density of 50,000 cells/well. Antisense oligonucleotide (AON) to TDP43 was transfected with Endoporter (Gene Tools, Philomath, OR, USA) to decrease expression of the full length UNC13a transcript. Vehicle control consisted of motor neuron treatment with Endoporter alone. Positive controls included cells that were treated with TDP43 AON alone (“AON TDP43” or “TDP43 AON”).

TDP43 AON is a gapmer oligonucleotide and has the following sequence and chemistry:

(SEQ ID NO: 1669)
5′ A*A*G*G*C*T*T*C*A*T*A*T*T*G*T*A*C*T*T*T 3′

where *=phosphorothioate, underlined=DNA, other=2′-MOE RNA; each “C” is 5-MeC.

To evaluate UNC13a AON ability to restore full length UNC13A (UNC13A FL) mRNA (also referred to as correctly spliced UNC13A (UNC13A CS) mRNA), antisense oligonucleotides to UNC13A were co-incubated with TDP43 AON in Endoporter in media before addition to the cells. After 72 hours, antisense oligonucleotides and Endoporter were washed out and replaced with fresh media. After 72 additional hours, RNA was collected from the 96-well plates for RT-qPCR. RNA was isolated, cDNA generated, and multiplexed RT-qPCR assay performed with Taqman probes for UNC13A full length transcript and reference GAPDH quantification.

Transcript levels (e.g., UNC13A full length transcript or TDP43 transcript) were detected by RT-qPCR using Taqman. Specifically, RT-qPCR was performed for detecting GAPDH using Thermofisher® TaqMan Gene Expression Assay Hs03929097_g1. RT-qPCR was performed for detecting UNC13aFL transcripts using Thermofisher® TaqMan Gene Expression Assay Hs00392638_m1.

RT-qPCR was performed on Applied Biosystems® 7500 Real-time PCR systems. One cycle of reverse transcription was performed at a temperature of 50° C. for 5 min. One cycle of RT inactivation/initial denaturation was performed at a temperature of 95° C. for 20 seconds. Forty five cycles of amplification were performed at a temperature of 95° C. for 1 second followed by 60° C. for 20 seconds.

UNC13A-FL (Ct) was normalized to GAPDH (deltaCt). To visualize the quantitative changes (e.g., % decrease of UNC13A-FL), the normalized UNC13A-FL signal was further normalized to the vehicle (treated with Endoporter alone, deltadeltaCt). Relative quantity (RQ) of transcript level was calculated using the equation RQ=2^{-deltadeltaCt} and is used to describe the treatment condition comparison to normal, healthy levels (1.0). RQ values for UNC13A FL were normalized using the following formula:

$$( ( ( RQAON - RQTDP 43 ) / ( R Q e n d o - R Q T D P 43 ) ) * 100$$

Table 35B shows the RT-qPCR results of UNC13A AONs with spacers and performance of UNC13A AONs without spacers in human motor neurons. UNC13A AONs (e.g., UNC13A oligonucleotides without spacers or with one or two spacers) were tested for their ability to increase or restore fill-length UNC13A mRNA (i.e., mRNA from which full-length UNC13A protein is translated) levels. In some cases, UNC13A AONs with spacers increased full-length UNC13A mRNA (“UNC13A FL”), also referred to herein as correctly spliced UNC13A (UNC13A CS). In some cases, UNC13A AONs without spacers increased full-length UNC13A mRNA or correctly spliced UNC13A mRNA. Specific AON sequences are labeled according to their corresponding SEQ ID NO.

As shown in Table 35B, a 500 nM dose of SEQ ID NO: 9683 (ACACAAASGGCCCAASCCTGAGT with two spacers) rescued full length UNC13A mRNA to 55.690%. Comparatively, a 500 nM dose of SEQ ID NO: 7318 (ACACAAACGGCCCAATCCTGAGT with no spacers) rescued full length UNC13A mRNA to 41.869%. This indicates that the addition of spacers of SEQ ID NO: 9683 improves the performance in comparison to the UNC13A AON counterpart without spacers (e.g., SEQ ID NO: 7318).

Additionally shown in Table 35B, a 200 nM dose of SEQ ID NO: 9689 (GATCCGCSACTAATSCACCTAC with two spacers) rescued full length UNC13A mRNA to 55.876%. A 500 nM dose of SEQ ID NO: 9689 (GATCCGCSACTTAATSCACCTAC with two spacers) rescued full length UNC13A mRNA to 66.616%. Comparatively, a 200 nM dose of SEQ ID NO: 8077 (GATCCGCAACTAATCCACCTAC with no spacers) rescued full length UNC13A mRNA to 43.878%. A 500 nM dose of SEQ ID NO: 8077 (GATCCGCAACTAATCCACCTAC with no spacers) rescued full length UNC13A mRNA to 66.9823%. This indicates that at 200 nM and 500 nM doses, the performance of an AON with SEQ ID NO: 9689 (with spacers) is similar to the performance of the UNC13A AON counterpart without spacers (e.g., SEQ ID NO: 8077).

Additionally shown in Table 35B, a 200 nM dose of SEQ ID NO: 9692 (CACCCACSCATCTAASTACCCCA with two spacers) rescued full length UNC13A mRNA to 47.943%. A 500 nM dose of SEQ ID NO: 9692 (CACCCACSCATCTAASTACCCCA with two spacers) rescued full length UNC13A mRNA to 83.190%. Comparatively, a 200 nM dose of SEQ ID NO: 8182 (CACCCACCCATCTAACTACCCCA with no spacers) rescued full length UNC13A mRNA to 47.681%. A 500 nM dose of SEQ ID NO: 8182 (CACCCACCCATCTAACTACCCCA with no spacers) rescued full length UNC13A mRNA to 41.1564%. This indicates that at a 200 nM dose, the performance of SEQ ID NO: 9692 (with spacers) is similar to the performance of the UNC13A AON counterpart without spacers (e.g., SEQ ID NO: 8182). However, at a 500 nM dose, the addition of spacers of SEQ ID NO: 9692 improves the performance in comparison to the UNC13A AON counterpart without spacers (e.g., SEQ ID NO: 8182).

Additionally shown in Table 35B, a 500 nM dose of SEQ ID NO: 9691 (CCCACCCSTCTAACTSCCCCAAA with two spacers) rescued full length UNC13A mRNA to 43.097%. Comparatively, a 500 nM dose of SEQ ID NO: 8180 (CCCACCCATCTAACTACCCCAAA with no spacers) rescued full length UNC13A mRNA to 23.368%. This indicates that at a 500 nM dose, the addition of spacers improves the performance of an AON with SEQ ID NO: 9691 in comparison to the UNC13A AON counterpart without spacers (e.g., SEQ ID NO: 8180).

Additionally shown in Table 35B, a 200 nM dose of SEQ ID NO: 9694 (AAGTGTCSTGGAGAGSGCAAGGG with two spacers) rescued full length UNC13A mRNA to 35.388%. A 500 nM dose of SEQ ID NO: 9694 (AAGTGTCSTGGAGAGSGCAAGGG with two spacers) rescued full length UNC13A mRNA to 6.602%. Comparatively, a 200 nM dose of SEQ ID NO: 8304 (AAGTGTCATGGAGAGTGCAAGGG with no spacers) rescued full length UNC13A mRNA to 19.044%. A 500 nM dose of SEQ ID NO: 8304 (AAGTGTCATGGAGAGTGCAAGGG with no spacers) rescued full length UNC13A mRNA to −34.581%. This indicates that at 200 nM and 500 nM doses, the addition of spacers improves the performance of an AON with SEQ ID NO: 9694 in comparison to the UNC13A AON counterpart without spacers (e.g., SEQ ID NO: 8304).

Additionally shown in Table 35B, a 200 nM dose of SEQ ID NO: 9672 (GAAAGTGTSATGGAGAGTSCAAGGG with two spacers) rescued full length UNC13A mRNA to 67.769%. A 500 nM dose of SEQ ID NO: 9672 (GAAAGTGTSATGGAGAGTSCAAGGG with two spacers) rescued full length UNC13A mRNA to 84.785%. Comparatively, a 200 nM dose of SEQ ID NO: 5778 (GAAAGTGTCATGGAGAGTGCAAGGG with no spacers) rescued full length UNC13A mRNA to 22.475%. A 500 nM dose of SEQ ID NO: 5778 (GAAAGTGTCATGGAGAGTGCAAGGG with no spacers) rescued full length UNC13A mRNA to 24.836%. This indicates that at 200 nM and 500 nM doses, the addition of spacers improves the performance of an AON with SEQ ID NO: 9672 in comparison to the UNC13A AON counterpart without spacers (e.g., SEQ ID NO: 5778).

Altogether, these results demonstrate that different UNC13A AONs including two spacers are capable of increasing UNC13A-FL mRNA to levels that are comparable to or improved beyond their non-spacer counterparts.

TABLE 35B
Performance of UNC13A AONs (UNC13A oligonucleotides without spacers, or with one or
two spacers) for restoring UNC13A FL.

| | Oligonucleotide Sequence* | QPCR potency | | QPCR potency | |
|---|---|---|---|---|---|
| SEQ ID | (where S indicates presence | 200 nM | 200 nM | 500 nM | 500 nM |
| NO: | of a Spacer) (5′→3′) | Normalized | RQ | Normalized | RQ |
| 4572 | GAAGAGCTGGGCAGGCAGGAATGGT | 3.79367 | 0.480565 | −63.053 | 0.19433 |
| 4596 | TGTGGATGGTGTGGCCAGAAAGAGG | 60.6986 | 0.724229 | −23.435 | 0.36397 |
| 4607 | GGCCAGGAGAGTGTGGATGGTGTGG | 154.828 | 1.127288 | 434.631 | 2.32539 |
| 4631 | ATGGCATGGCGGGCAGTCTCAGAGG | 37.1601 | 0.448286 | 12.6634 | 0.41631 |
| 4638 | AAAGGGAATGGCATGGCGGGCAGTC | −26.139 | 0.13718 | 22.9464 | 0.49028 |
| 4642 | AGGTAAAGGGAATGGCATGGCGGGC | −5.3242 | 0.383369 | −37.199 | 0.17903 |
| 7169 | GGTAAAGGGAATGGCATGGCGGG | −29.228 | 0.227001 | −33.566 | 0.20089 |
| 9678 | GGTAAAGSGAATGGCSTGGCGGG | −16.331 | 0.304608 | −29.739 | 0.22392 |
| 7170 | AGGTAAAGGGAATGGCATGGCGG | −13.385 | 0.322337 | −40.436 | 0.15956 |
| 9677 | AGGTAAASGGAATGGSATGGCGG | −13.664 | 0.320657 | −24.027 | 0.2583 |
| 7168 | GTAAAGGGAATGGCATGGCGGGC | −29.989 | 0.222419 | −26.677 | 0.24235 |
| 9676 | GTAAAGGSAATGGCASGGCGGGC | 25.771 | 0.557959 | −18.428 | 0.29199 |
| 4645 | TCCAGGTAAAGGGAATGGCATGGCG | −7.8349 | 0.268854 | 40.2877 | 0.61503 |
| 4657 | AGGGAGGAGTTTTCCAGGTAAAGGG | −32.255 | 0.392785 | −25.109 | 0.4256 |
| 9664 | AGGGAGSAGTTTTCCAGGSAAAGGG | 6.97605 | 0.572906 | −3.6889 | 0.52394 |
| 9669 | AGGGAGGASTTTTCCAGGSAAAGGG | −70.51 | 0.217148 | −78.696 | 0.17957 |
| 4692 | AGAGGAGGTGACCCTGAATCTGGAC | 129.033 | 0.93262 | 110.34 | 1.0505 |
| 7219 | GAGGAGGTGACCCTGAATCTGGA | 169.855 | 1.024819 | 164.814 | 1.30762 |
| 9681 | GAGGAGGSGACCCTGSATCTGGA | −72.457 | 0.658323 | 138.629 | 1.28191 |
| 7220 | AGAGGAGGTGACCCTGAATCTGG | 171.498 | 1.135638 | 3.26987 | 0.54512 |
| 9680 | AGAGGAGSTGACCCTSAATCTGG | 7.49253 | 0.565048 | −57.946 | 0.25617 |
| 7218 | AGGAGGTGACCCTGAATCTGGAC | 44.7095 | 0.740715 | 125.001 | 1.1197 |
| 9679 | AGGAGGTSACCCTGASTCTGGAC | −44.26 | 0.320771 | 12.8179 | 0.59018 |
| 4725 | GAGAGTCAACCTGGAGGCCAAGGTG | 32.5688 | 0.559505 | 57.0338 | 0.7355 |
| 4730 | GTAGTGAGAGTCAACCTGGAGGCCA | 70.681 | 0.905428 | 67.6819 | 0.88671 |
| 4733 | TGAGTAGTGAGAGTCAACCTGGAGG | 91.2119 | 1.033573 | 59.7857 | 0.83742 |
| 4774 | CCTGAGTGGTTAGGGCTGGAATAGA | 46.7792 | 0.756244 | −24.332 | 0.3124 |
| 4778 | CAATCCTGAGTGGTTAGGGCTGGAA | −8.5 | 0.411217 | 73.8646 | 0.9253 |
| 4784 | ACGGCCCAATCCTGAGTGGTTAGGG | 66.8521 | 0.88153 | 45.3726 | 0.74746 |
| 4790 | ACACAAACGGCCCAATCCTGAGTGG | −11.42 | 0.462541 | 75.8487 | 0.8839 |
| 7317 | CACAAACGGCCCAATCCTGAGTG | −11.128 | 0.463949 | −41.939 | 0.31518 |
| 9684 | CACAAACSGCCCAATSCTGAGTG | 50.7872 | 0.762897 | −14.314 | 0.44857 |
| 7318 | ACACAAACGGCCCAATCCTGAGT | −27.43 | 0.385239 | 41.8694 | 0.71984 |
| 9683 | ACACAAASGGCCCAASCCTGAGT | −14.732 | 0.44655 | 55.6902 | 0.78657 |
| 7316 | ACAAACGGCCCAATCCTGAGTGG | −49.751 | 0.277462 | −20.306 | 0.41964 |
| 9682 | ACAAACGSCCCAATCSTGAGTGG | 105.349 | 1.026341 | 47.8744 | 0.74883 |
| 4793 | CAGACACAAACGGCCCAATCCTGAG | −40.7843 | 0.3272 | −5.4793 | 0.5486 |
| 4799 | CATACCCAGACACAAACGGCCCAAT | −21.2475 | 0.3317 | 14.4015 | 0.5542 |
| 4803 | GAGACATACCCAGACACAAACGGCC | 47.2141 | 0.7590 | 116.3656 | 1.1906 |
| 4813 | GCAGCTGGAAGAGACATACCCAGAC | 4.7146 | 0.5625 | −66.6389 | 0.2349 |
| 9665 | GCAGCTSGAAGAGACATASCCAGAC | 33.7840 | 0.6960 | −6.4957 | 0.5111 |
| 9670 | GCAGCTGGSAGAGACATASCCAGAC | 3.7461 | 0.5581 | 35.3538 | 0.7032 |
| 4831 | TCTTTCCAGGAAACCCAGGCAGCTG | 17.5514 | 0.5395 | 30.5680 | 0.5952 |
| 9741 | GTTCTTTCCAGGAAACCCAGGCAGC | 41.1141 | 0.5932 | −27.3254 | 0.2666 |
| 7360 | TTCTTTCCAGGAAACCCAGGCAG | −31.8396 | 0.2450 | −7.7732 | 0.3599 |
| 9687 | TTCTTTCSAGGAAACSCAGGCAG | −72.0800 | 0.0530 | −37.1813 | 0.2196 |
| 7361 | GTTCTTTCCAGGAAACCCAGGCA | −14.5643 | 0.3275 | 44.7209 | 0.6104 |
| 9686 | GTTCTTTSCAGGAAASCCAGGCA | −38.7597 | 0.2120 | −22.4863 | 0.2897 |
| 7359 | TCTTTCCAGGAAACCCAGGCAGC | 2.0002 | 0.4065 | −15.1244 | 0.3248 |
| 9685 | TCTTTCCSGGAAACCSAGGCAGC | −59.9899 | 0.1107 | −64.5153 | 0.0891 |
| 4984 | CACGAATCTACCCACCAACTCATCC | −39.5824 | 0.2948 | −56.2924 | 0.2233 |
| 4990 | TCTAGCCACGAATCTACCCACCAAC | 75.4170 | 0.7873 | −12.0685 | 0.4126 |
| 5457 | GTCTGTCCACCAGCCACTCACAACC | 121.2337 | 0.9834 | −62.7895 | 0.1955 |
| 5460 | TTCGTCTGTCCACCAGCCACTCACA | −18.2285 | 0.3863 | −45.7096 | 0.2686 |
| 5467 | TCCATTTTTCGTCTGTCCACCAGCC | −63.8195 | 0.2479 | −54.2647 | 0.2917 |
| 5549 | GATCCGCAACTTAATCCACCTACCC | 88.0305 | 0.9091 | 44.9905 | 0.5436 |
| 8076 | ATCCGCAACTTAATCCACCTACC | 30.7027 | 0.4223 | 45.1113 | 0.5446 |
| 9690 | ATCCGCASCTTAATCSACCTACC | 38.8278 | 0.4913 | 50.8796 | 0.5936 |
| 8077 | GATCCGCAACTTAATCCACCTAC | 43.8789 | 0.5342 | 66.9823 | 0.7303 |
| 9689 | GATCCGCSACTTAATSCACCTAC | 55.8768 | 0.6360 | 66.6162 | 0.7272 |
| 8075 | TCCGCAACTTAATCCACCTACCC | 64.3790 | 0.7082 | 67.3066 | 0.7331 |
| 9688 | TCCGCAASTTAATCCSCCTACCC | 53.4236 | 0.6152 | 51.3988 | 0.5980 |
| 5654 | CACCCACCCATCTAACTACCCCAAA | 64.1844 | 0.6729 | 44.6783 | 0.4941 |
| 8181 | ACCCACCCATCTAACTACCCCAA | 41.2908 | 0.4631 | 64.1844 | 0.6729 |
| 9693 | ACCCACCSATCTAACSACCCCAA | 46.9810 | 0.4070 | 58.8250 | 0.6238 |
| 8182 | CACCCACCCATCTAACTACCCCA | 47.6810 | 0.5217 | 41.0564 | 0.4609 |
| 9692 | CACCCACSCATCTAASTACCCCA | 47.9432 | 0.5241 | 83.1895 | 0.8471 |
| 8180 | CCCACCCATCTAACTACCCCAAA | 72.0651 | 0.5775 | 23.3677 | 0.2988 |
| 9691 | CCCACCCSTCTAACTSCCCCAAA | 41.8445 | 0.4682 | 43.0965 | 0.4796 |
| 5778 | GAAAGTGTCATGGAGAGTGCAAGGG | 22.4754 | 0.5041 | 24.8358 | 0.5167 |
| 8305 | AAAGTGTCATGGAGAGTGCAAGG | 82.5919 | 0.8245 | 7.0300 | 0.4218 |
| 9696 | AAAGTGTSATGGAGASTGCAAGG | 61.0070 | 0.7095 | −4.8799 | 0.3583 |
| 8306 | GAAAGTGTCATGGAGAGTGCAAG | 55.7844 | 0.6816 | −19.7864 | 0.2789 |
| 9695 | GAAAGTGSCATGGAGSGTGCAAG | 40.0402 | 0.5977 | 3.6974 | 0.4040 |
| 8304 | AAGTGTCATGGAGAGTGCAAGGG | 19.0435 | 0.4858 | −34.5807 | 0.2001 |
| 9694 | AAGTGTCSTGGAGAGSGCAAGGG | 35.3880 | 0.5729 | 6.6020 | 0.4195 |
| 9667 | GAAAGTSTCATGGAGAGTSCAAGGG | −10.6183 | 0.3278 | 50.6900 | 0.6545 |
| 9672 | GAAAGTGTSATGGAGAGTSCAAGGG | 67.7691 | 0.7455 | 84.7852 | 0.8362 |
| 5790 | CACCATGGGAGAGAAAGTGTCATGG | 17.7544 | 0.4529 | 64.3335 | 0.7880 |

*Unless otherwise noted, each of the nucleosides of antisense oligonucleotides shown in Table 35B are modified nucleosides with 2′-O-(2-methoxyethyl) (2′-MOE) sugar moieties, each “C” is replaced with a 5-methylcytosine (5-MeC), and all internucleoside linkages are phosphorothioan linkages. A spacer as indicated by S is not a nucleoside. S represents a spacer of Formula (Iia′) as disclosed herein.

### Example 12: Methods for Evaluating UNC13A Antisense Oligonucleotides

UNC13A antisense oligonucleotides were evaluated in iPSC derived human motor neurons (hMN). The cells were seeded in 96-well plates at a density of 40,000 cells/well. Antisense oligonucleotide (AON) to TDP43 was transfected with Endoporter (Gene Tools, Philomath, OR, USA) to decrease expression of the full length UNC13A transcript and increase expression of UNC13A cryptic exon. Vehicle control consisted of motor neuron treatment with Endoporter alone. Positive controls included cells that were treated with TDP43 AON alone (“AON TDP43” or “TDP43 AON”).

TDP43 AON is a gapmer oligonucleotide and has the following sequence and chemistry:

(SEQ ID NO: 1669)
5′ A*A*G*G*C*T*T*C*A*T*A*T*T*G*T*A*C*T*T*T 3′

where *=phosphorothioate, underlined=DNA, other=2′-MOE RNA; each “C” is 5-MeC.

To evaluate UNC13A AON ability to reduce UNC13A cryptic exon levels, antisense oligonucleotides to UNC13A were co-incubated with TDP43 AON in Endoporter in media before addition to the cells. After 72 hours, antisense oligonucleotides and Endoporter were washed out and replaced with fresh media alone. After six additional days, RNA was collected from the 96-well plates for RT-qPCR. RNA was isolated, cDNA generated and multiplexed RT-qPCR assay performed with Taqman probes for UNC13A cryptic exon, and reference GAPDH quantification.

Transcript levels (e.g., UNC13A cryptic exon, and TDP43 transcript) were detected by RT-qPCR using Taqman. Specifically, RT-qPCR was performed for detecting GAPDH using Thermofisher® TaqMan Gene Expression Assay Hs03929097_g1. UNC13a cryptic exon was detected using custom sequences.

UNC13a Cryptic Exon:
Forward Primer:
(SEQ ID NO: 10780)
ATTGTTCTGCACGTCGGT
Reverse Primer:
(SEQ ID NO: 10781)
GTCTGGGTATGTCTCTTCCAG
Probe Sequence:
(SEQ ID NO: 10782)
AGTTCTTTCCAGGAAACCCAGGCA

RT-qPCR was performed on Applied Biosystems® 7500 Real-time PCR systems. One cycle of reverse transcription was performed at a temperature of 50° C. for 5 min. One cycle of RT inactivation/initial denaturation was performed at a temperature of 95° C. for 20 seconds. Forty five cycles of amplification were performed at a temperature of 95° C. for 1 second followed by 60° C. for 20 seconds.

UNC13A-cryptic (Ct) was normalized to GAPDH (deltaCt). To visualize the quantitative changes (e.g., % decrease of UNC13A-cryptic), the normalized UNC13A-cryptic signal was further normalized to the vehicle (treated with Endoporter alone, deltadeltaCt). Relative quantity (RQ) of transcript level was calculated using the equation RQ=2^{-deltadeltaCt} and is used to describe the treatment condition comparison to normal, healthy levels (1.0). RQ values for UNC13A cryptic were normalized using the following formula:

$$( ( ( RQAON - RQendo ) / ( RQTDP 43 - RQendo ) ) * 100$$

Table 36 shows the RT-qPCR results of UNC13A AONs with spacers and performance of UNC13A AONs without spacers in human motor neurons.

As shown in Table 36, UNC13A AONs (e.g., UNC13A oligonucleotides without spacers or with one or two spacers) were tested for their ability to reduce UNC13A transcripts with a cryptic exon. In some cases, UNC13A AONs with spacers reduced UNC13A cryptic exon levels. In some cases, UNC13A AONs without spacers reduced UNC13A cryptic exon levels. Specific AON sequences are labeled according to their corresponding SEQ ID NO.

As shown in Table 36, a 200 nM dose of SEQ ID NO: 4803 (GAGACATACCCAGACACAAACGGCC with no spacers) reduced UNC13A cryptic exon levels to 4.4%. A 50 nM dose of SEQ ID NO: 4803 (GAGACATACCCAGACACAAACGGCC with no spacers) reduced UNC13A cryptic exon levels to 20.3%.

As shown in Table 36, a 200 nM dose of SEQ ID NO: 7361 (GTTCTTTCCAGGAAACCCAGGCA with no spacers) reduced UNC13A cryptic exon levels to 5.1%. A 50 nM dose of SEQ ID NO: 7361 (GTTCTTTCCAGGAAACCCAGGCA with no spacers) reduced UNC13A cryptic exon levels to 26.0%.

As shown in Table 36, a 200 nM dose of SEQ ID NO: 9686 (GTTCTTTSCAGGAAASCCAGGCA with two spacers) reduced UNC13A cryptic exon levels to 6.6%. A 50 nM dose of SEQ ID NO: 9686 (GTTCTTTSCAGGAAASCCAGGCA with two spacers) reduced UNC13A cryptic exon levels to 41.2%.

As shown in Table 36, a 200 nM dose of SEQ ID NO: 9670 (GCAGCTGGSAGAGACATASCCAGAC with two spacers) reduced UNC13A cryptic exon levels to 8.3%. A 50 nM dose of SEQ ID NO: 9670 (GCAGCTGGSAGAGACATASCCAGAC with two spacers) reduced UNC13A cryptic exon levels to 31.0%.

As shown in Table 36, a 200 nM dose of SEQ ID NO: 4833 (GTTCTTTCCAGGAAACCCAGGCAGC with no spacers) reduced UNC13A cryptic exon levels to 8.8%. A 50 nM dose of SEQ ID NO: 4833 (GTTCTTTCCAGGAAACCCAGGCAGC with no spacers) reduced UNC13A cryptic exon levels to 35.4%.

As shown in Table 36, a 200 nM dose of SEQ ID NO: 9655 (GCAGCTSGAAGAGACATASCCAGAC with two spacers) reduced UNC13A cryptic exon levels to 11.8%. A 50 nM dose of SEQ ID NO: 9655 (GCAGCTSGAAGAGACATASCCAGAC with two spacers) reduced UNC13A cryptic exon levels to 30.6%.

As shown in Table 36, a 200 nM dose of SEQ ID NO: 4813 (GCAGCTGGAAGAGACATACCCAGAC with no spacers) reduced UNC13A cryptic exon levels to 13.8%. A 50 nM dose of SEQ ID NO: 4813 (GCAGCTGGAAGAGACATACCCAGAC with no spacers) reduced UNC13A cryptic exon levels to 66.0%.

As shown in Table 36, a 200 nM dose of SEQ ID NO: 7360 (TTCTTTCCAGGAAACCCAGGCAG with no spacers) reduced UNC13A cryptic exon levels to 15.2%. A 50 nM dose of SEQ ID NO: 7360 (TTCTTTCCAGGAAACCCAGGCAG with no spacers) reduced UNC13A cryptic exon levels to 34.4%.

As shown in Table 36, a 200 nM dose of SEQ ID NO: 8115 (ACATCCATCCATCCATCCATTCA with no spacers) reduced UNC13A cryptic exon levels to 15.8%. A 50 nM dose of SEQ ID NO: 8115 (ACATCCATCCATCCATCCATTCA with no spacers) reduced UNC13A cryptic exon levels to 45.4%.

As shown in Table 36, a 200 nM dose of SEQ ID NO: 9685 (TCTTTCCSGGAAACCSAGGCAGC with two spacers) reduced UNC13A cryptic exon levels to 18.2%. A 50 nM dose of SEQ ID NO: 9685 (TCTTTCCSGGAAACCSAGGCAGC with two spacers) reduced UNC13A cryptic exon levels to 41.9%.

As shown in Table 36, a 200 nM dose of SEQ ID NO: 9687 (TTCTTTCSAGGAAACSCAGGCAG with two spacers) reduced UNC13A cryptic exon levels to 20.1%. A 50 nM dose of SEQ ID NO: 9687 (TTCTTTCSAGGAAACSCAGGCAG with two spacers) reduced UNC13A cryptic exon levels to 42.8%.

As shown in Table 36, a 200 nM dose of SEQ ID NO: 5589 (ACACATCCATCCATCCATCCATTCA with no spacers) reduced UNC13A cryptic exon levels to 23.1%. A 50 nM dose of SEQ ID NO: 5589 (ACACATCCATCCATCCATCCATTCA with no spacers) reduced UNC13A cryptic exon levels to 50.2%.

As shown in Table 36, a 200 nM dose of SEQ ID NO: 7359 (TCTTTCCAGGAAACCCAGGCAGC with no spacers) reduced UNC13A cryptic exon levels to 25%. A 50 nM dose of SEQ ID NO: 7359 (TCTTTCCAGGAAACCCAGGCAGC with no spacers) reduced UNC13A cryptic exon levels to 40.6%.

As shown in Table 36, a 200 nM dose of SEQ ID NO: 4799 (CATACCCAGACACAAACGGCCCAAT with no spacers) reduced UNC13A cryptic exon levels to 27.2%. A 50 nM dose of SEQ ID NO: 4799 (CATACCCAGACACAAACGGCCCAAT with no spacers) reduced UNC13A cryptic exon levels to 43.4%.

As shown in Table 36, a 200 nM dose of SEQ ID NO: 5672 (CTCTTTTATCCATCCACACACCCAC with no spacers) reduced UNC13A cryptic exon levels to 34.4%. A 50 nM dose of SEQ ID NO: 5672 (CTCTTTTATCCATCCACACACCCAC with no spacers) reduced UNC13A cryptic exon levels to 64.3%.

As shown in Table 36, a 200 nM dose of SEQ ID NO: 8200 (CTCTTTTATCCATCCACACACCC with no spacers) reduced UNC13A cryptic exon levels to 40.5%. A 50 nM dose of SEQ ID NO: 8200 (CTCTTTTATCCATCCACACACCC with no spacers) reduced UNC13A cryptic exon levels to 82.2%.

As shown in Table 36, a 200 nM dose of SEQ ID NO: 4831 (TCTTTCCAGGAAACCCAGGCAGCTG with no spacers) reduced UNC13A cryptic exon levels to 41.6%. A 50 nM dose of SEQ ID NO: 4831 (TCTTTCCAGGAAACCCAGGCAGCTG with no spacers) reduced UNC13A cryptic exon levels to 54.2%.

As shown in Table 36, a 200 nM dose of SEQ ID NO: 9695 (GAAAGTGSCATGGAGSGTGCAAG with two spacers) reduced UNC13A cryptic exon levels to 42.0%. A 50 nM dose of SEQ ID NO: 9695 (GAAAGTGSCATGGAGSGTGCAAG with two spacers) reduced UNC13A cryptic exon levels to 54.0%.

As shown in Table 36, a 200 nM dose of SEQ ID NO: 10670 (ATCTACSCTTTTATCCATSCACACA with two spacers) reduced UNC13A cryptic exon levels to 44.5%. A 50 nM dose of SEQ ID NO: 10670 (ATCTACSCTTTTATCCATSCACACA with two spacers) reduced UNC13A cryptic exon levels to 45.5%.

As shown in Table 36, a 200 nM dose of SEQ ID NO: 5677 (ATCTACTCTTTTATCCATCCACACA with no spacers) reduced UNC13A cryptic exon levels to 46.4%. A 50 nM dose of SEQ ID NO: 5677 (ATCTACTCTTTTATCCATCCACACA with no spacers) reduced UNC13A cryptic exon levels to 69.7%.

As shown in Table 36, a 200 nM dose of SEQ ID NO: 8177 (ACACATCCATCCATCCATCCATT with no spacers) reduced UNC13A cryptic exon levels to 47.8%.

As shown in Table 36, a 200 nM dose of SEQ ID NO: 10671 (CTACTCTSTTATCCASCCACACA with two spacers) reduced UNC13A cryptic exon levels to 48.1%. A 50 nM dose of SEQ ID NO: 10671 (CTACTCTSTTATCCASCCACACA with two spacers) reduced UNC13A cryptic exon levels to 76.5%.

As shown in Table 36, a 200 nM dose of SEQ ID NO: 10672 (ATCTACTCSTTTATCCATSCACACA with two spacers) reduced UNC13A cryptic exon levels to 48.2%. A 50 nM dose of SEQ ID NO: 10672 (ATCTACTCSTTTATCCATSCACACA with two spacers) reduced UNC13A cryptic exon levels to 62.8%.

As shown in Table 36, a 200 nM dose of SEQ ID NO: 10673 (CTTTCAGSAATTCAASCACACAT with two spacers) reduced UNC13A cryptic exon levels to 49.6%. A 50 nM dose of SEQ ID NO: 10673 (CTTTCAGSAATTCAASCACACAT with two spacers) reduced UNC13A cryptic exon levels to 70.9%.

As shown in Table 36, a 200 nM dose of SEQ ID NO: 3774 (AAGTGTCATGGAGAGTGCAAGGG with no spacers) reduced UNC13A cryptic exon levels to 49.8%. A 50 nM dose of SEQ ID NO: 3774 (AAGTGTCATGGAGAGTGCAAGGG with no spacers) reduced UNC13A cryptic exon levels to 67.0%.

TABLE 36
Performance of Exemplary UNC13A AONs evaluated in human derived iPSC motor neurons

| | | QPCR potency | | QPCR potency | |
|---|---|---|---|---|---|
| | | 200 nM | 200 nM | 50 nM | 50 nM |
| | Oligonucleotide Sequence* | Normalized | SD Normalized | Normalized | SD Normalized |
| SEQ ID | (where S indicates presence | UNC13A | UNC13A | UNC13A | UNC13A |
| NO: | of a Spacer) (5′→3′) | Cryptic Exon | Cryptic Exon | Cryptic Exon | Cryptic Exon |
| 4803 | GAGACATACCCAGACACAAACGGCC | 4.38 | 4.41 | 20.3 | 14.4 |
| 7361 | GTTCTTTCCAGGAAACCCAGGCA | 5.11 | 4.14 | 26 | 15.2 |
| 9686 | GTTCTTTSCAGGAAASCCAGGCA | 6.62 | 6.06 | 41.2 | 15.2 |
| 9670 | GCAGCTGGSAGAGACATASCCAGAC | 8.3 | 7.86 | 31 | 26.4 |
| 4833 | GTTCTTTCCAGGAAACCCAGGCAGC | 8.84 | 7.85 | 35.4 | 22.4 |
| 9655 | GCAGCTSGAAGAGACATASCCAGAC | 11.8 | 10.7 | 30.6 | 13 |
| 4813 | GCAGCTGGAAGAGACATACCCAGAC | 13.8 | 7.11 | 66 | 45.9 |
| 7360 | TTCTTTCCAGGAAACCCAGGCAG | 15.2 | 7.39 | 34.4 | 25.1 |
| 8115 | ACATCCATCCATCCATCCATTCA | 15.8 | 8.84 | 45.4 | 14 |
| 9685 | TCTTTCCSGGAAACCSAGGCAGC | 18.2 | 9.18 | 41.9 | 24.2 |
| 9687 | TTCTTTCSAGGAAACSCAGGCAG | 20.1 | 9.33 | 42.8 | 27.7 |
| 5589 | ACACATCCATCCATCCATCCATTCA | 23.1 | 15.7 | 50.2 | 21.5 |
| 7359 | TCTTTCCAGGAAACCCAGGCAGC | 25 | 11.7 | 40.6 | 22.9 |
| 4799 | CATACCCAGACACAAACGGCCCAAT | 27.2 | 20.6 | 43.4 | 28 |
| 5672 | CTCTTTTATCCATCCACACACCCAC | 34.4 | 20.1 | 64.3 | 34.7 |
| 8200 | CTCTTTTATCCATCCACACACCC | 40.5 | 15.2 | 82.2 | 45.5 |
| 4831 | TCTTTCCAGGAAACCCAGGCAGCTG | 41.6 | 30.5 | 54.2 | 20.8 |
| 9695 | GAAAGTGSCATGGAGSGTGCAAG | 42 | 38.1 | 54 | 32.5 |
| 10670 | ATCTACSCTTTTATCCATSCACACA | 44.5 | 22.4 | 45.5 | 34.4 |
| 5677 | ATCTACTCTTTTATCCATCCACACA | 46.4 | 29.9 | 69.7 | 47.6 |
| 8177 | ACACATCCATCCATCCATCCATT | 47.8 | 24.9 | 122 | 41.9 |
| 10671 | CTACTCTSTTATCCASCCACACA | 48.1 | 35.4 | 76.5 | 37.9 |
| 10672 | ATCTACTCSTTTATCCATSCACACA | 48.2 | 21.5 | 62.8 | 44.3 |
| 10673 | CTTTCAGSAATTCAASCACACAT | 49.6 | 39.2 | 70.9 | 46.3 |
| 3774 | AAGTGTCATGGAGAGTGCAAGGG | 49.8 | 39.3 | 67 | 59.7 |
| 8116 | CACATCCATCCATCCATCCATTC | 51.6 | 14.2 | 80.3 | 41.4 |
| 4778 | CAATCCTGAGTGGTTAGGGCTGGAA | 54.2 | 18.8 | 59.6 | 16.2 |
| 4793 | CAGACACAAACGGCCCAATCCTGAG | 54.4 | 19.2 | 73.2 | 43.7 |
| 8198 | CTTTTATCCATCCACACACCCAC | 55.4 | 30.5 | 59 | 34.2 |
| 8199 | TCTTTTATCCATCCACACACCCA | 55.7 | 29.2 | 61.1 | 45.6 |
| 10674 | CTCTTTTSTCCATCCSCACACCC | 55.9 | 29.8 | 83.8 | 50.9 |
| 7092 | GGGCAGGCAGGAATGGTGAGTGG | 56.6 | 41 | 73.6 | 47.2 |
| 9671 | TCCACCAGSCACTCACAASCATCCA | 58 | 17.7 | 86.1 | 36.5 |
| 5654 | CACCCACCCATCTAACTACCCCAAA | 58.2 | 42.7 | 91 | 34.4 |
| 7091 | GGCAGGCAGGAATGGTGAGTGGA | 59.1 | 43.9 | 57.9 | 41.1 |
| 10675 | TCTTTTASCCATCCASACACCCA | 63.1 | 23.5 | 54.3 | 40.7 |
| 8182 | CACCCACCCATCTAACTACCCCA | 63.1 | 44.6 | 74.3 | 51.2 |
| 9696 | AAAGTGTSATGGAGASTGCAAGG | 63.7 | 39.2 | 55.5 | 53 |
| 10676 | TGCAATASTTCAACCSCACATCC | 64.1 | 25.7 | 75.7 | 42.9 |
| 9668 | GGGCAGGCSGGAATGGTGSGTGGAA | 64.2 | 32 | 73 | 39 |
| 4564 | GGGCAGGCAGGAATGGTGAGTGGAA | 64.6 | 42.2 | 91.2 | 48.4 |
| 9675 | GGCAGGCSGGAATGGSGAGTGGA | 64.6 | 49.5 | 92.8 | 65 |
| 10677 | CTCTTTSATCCATCCACASACCCAC | 65.1 | 34.1 | 53 | 32.2 |
| 3775 | AAAGTGTCATGGAGAGTGCAAGG | 65.3 | 45.4 | 71.2 | 64.2 |
| 10678 | CAGTAATSCAACCACSCATCCAT | 65.5 | 23.7 | 92.7 | 24.1 |
| 10679 | CTCTTTTASCCATCCACASACCCAC | 65.6 | 37.8 | 67.8 | 29.5 |
| 8203 | CTACTCTTTTATCCATCCACACA | 67.1 | 32 | 50.5 | 34 |
| 5608 | ACCTTTCAGTAATTCAACCACACAT | 67.5 | 52.3 | 87.4 | 58 |
| 8306 | GAAAGTGTCATGGAGAGTGCAAG | 68.3 | 52.7 | 52.7 | 38.7 |
| 10680 | CTTTTATSCATCCACSCACCCAC | 68.7 | 58.9 | 59.2 | 33.2 |
| 10681 | ACCTTTCASTAATTCAACSACACAT | 69.2 | 35.5 | 115 | 79.8 |
| 9683 | ACACAAASGGCCCAASCCTGAGT | 69.3 | 54.9 | 114 | 67.4 |
| 9692 | CACCCACSCATCTAASTACCCCA | 71.4 | 65.7 | 111 | 77.3 |
| 10682 | AGTAATTSAACCACASATCCATC | 72 | 16 | 58 | 21.7 |
| 9694 | AAGTGTCSTGGAGAGSGCAAGGG | 72.1 | 51.9 | 70.7 | 59.6 |
| 7318 | ACACAAACGGCCCAATCCTGAGT | 72.4 | 18.3 | 99.8 | 34.6 |
| 7916 | TGCAATAGTTCAACCACACATCC | 74 | 35.8 | 124 | 53.6 |
| 8134 | CTTTCAGTAATTCAACCACACAT | 74.1 | 39.5 | 65.7 | 57.3 |
| 4790 | ACACAAACGGCCCAATCCTGAGTGG | 74.3 | 16.6 | 109 | 53.2 |
| 4774 | CCTGAGTGGTTAGGGCTGGAATAGA | 75.2 | 42.7 | 92.4 | 35.7 |
| 8135 | CCTTTCAGTAATTCAACCACACA | 76.2 | 42.9 | 145 | 138 |
| 7317 | CACAAACGGCCCAATCCTGAGTG | 76.8 | 37.3 | 91.2 | 46.8 |
| 8181 | ACCCACCCATCTAACTACCCCAA | 77.1 | 52.6 | 87.2 | 59.1 |
| 10683 | TCTACTCSTTTATCCSTCCACAC | 78.2 | 43.6 | 88.6 | 71.7 |
| 9684 | CACAAACSGCCCAATSCTGAGTG | 78.2 | 45.6 | 86.8 | 45.4 |
| 9682 | ACAAACGSCCCAATCSTGAGTGG | 78.3 | 38.8 | 66.1 | 35 |
| 9666 | TCCACCSGCCACTCACAASCATCCA | 78.7 | 73.6 | 120 | 50.9 |
| 8180 | CCCACCCATCTAACTACCCCAAA | 78.9 | 50.3 | 71.8 | 60.4 |
| 9674 | GGGCAGGSAGGAATGSTGAGTGG | 82.7 | 67.3 | 142 | 100 |
| 10684 | CTTGCASTAGTTCAACCASACATCC | 86 | 34.4 | 140 | 132 |
| 10685 | ATCTACTSTTTTATCSATCCACA | 86.3 | 55.6 | 117 | 68.8 |
| 4784 | ACGGCCCAATCCTGAGTGGTTAGGG | 86.9 | 44.1 | 108 | 78 |
| 8077 | GATCCGCAACTTAATCCACCTAC | 88.3 | 76.5 | 115 | 86.5 |
| 9667 | GAAAGTSTCATGGAGAGTSCAAGGG | 89.6 | 50.5 | 107 | 42.8 |
| 8205 | ATCTACTCTTTTATCCATCCACA | 91.1 | 60.9 | 89.8 | 70.4 |
| 7316 | ACAAACGGCCCAATCCTGAGTGG | 92.9 | 43.5 | 78.5 | 47.3 |
| 7090 | GCAGGCAGGAATGGTGAGTGGAA | 94 | 74.1 | 118 | 82.7 |
| 10686 | ACCTTTCSGTAATTCSACCACAC | 95.2 | 44.9 | 60.6 | 52.1 |
| 4642 | AGGTAAAGGGAATGGCATGGCGGGC | 96 | 49.3 | 115 | 57 |
| 8075 | TCCGCAACTTAATCCACCTACCC | 96 | 82.6 | 111 | 77.3 |
| 5778 | GAAAGTGTCATGGAGAGTGCAAGGG | 96.3 | 58.9 | 118 | 77.8 |
| 10687 | CTTGCAASAGTTCAASCACACAT | 98.9 | 42.4 | 90.8 | 40.8 |
| 7918 | CTTGCAATAGTTCAACCACACAT | 99.5 | 39 | 95.7 | 46.1 |
| 9691 | CCCACCCSTCTAACTSCCCCAAA | 99.7 | 71.7 | 112 | 82.8 |
| 10688 | CAGTAATTSAACCACACASCCATCC | 101 | 95.2 | 128 | 130 |
| 7917 | TTGCAATAGTTCAACCACACATC | 102 | 54.4 | 138 | 55.5 |
| 8136 | ACCTTTCAGTAATTCAACCACAC | 105 | 105 | 120 | 108 |
| 10689 | GTAATTCSACCACACSTCCATCC | 106 | 60.1 | 116 | 48.6 |
| 4730 | GTAGTGAGAGTCAACCTGGAGGCCA | 107 | 21.8 | 92.6 | 27.2 |
| 8204 | TCTACTCTTTTATCCATCCACAC | 107 | 71.4 | 83.2 | 56.9 |
| 5790 | CACCATGGGAGAGAAAGTGTCATGG | 108 | 103 | 67.4 | 60.1 |
| 4657 | AGGGAGGAGTTTTCCAGGTAAAGGG | 109 | 50.5 | 95.7 | 21.4 |
| 9673 | GCAGGCASGAATGGTSAGTGGAA | 109 | 74.8 | 150 | 98.2 |
| 9693 | ACCCACCSATCTAACSACCCCAA | 112 | 74.4 | 81.6 | 45.8 |
| 10690 | ACACATSCATCCATCCATSCATTCA | 114 | 36.7 | 135 | 39.5 |
| 10691 | TTGCAATSGTTCAACSACACATC | 115 | 54.7 | 98.6 | 51.2 |
| 9664 | AGGGAGSAGTTTTCCAGGSAAAGGG | 116 | 30 | 110 | 29.8 |
| 10692 | ACATCCASCCATCCASCCATTCA | 116 | 50.6 | 111 | 38 |
| 10693 | ACCTTTSAGTAATTCAACSACACAT | 116 | 81.4 | 101 | 74.7 |
| 9672 | GAAAGTGTSATGGAGAGTSCAAGGG | 117 | 40.6 | 115 | 66.2 |
| 9669 | AGGGAGGASTTTTCCAGGSAAAGGG | 118 | 30.4 | 111 | 22.7 |
| 4725 | GAGAGTCAACCTGGAGGCCAAGGTG | 118 | 43.8 | 96.1 | 17.9 |
| 7909 | GTTCAACCACACATCCTTCCATT | 119 | 70.8 | 186 | 142 |
| 5390 | CTTGCAATAGTTCAACCACACATCC | 120 | 57.3 | 140 | 70.5 |
| 5457 | GTCTGTCCACCAGCCACTCACAACC | 120 | 104 | 154 | 122 |
| 7910 | AGTTCAACCACACATCCTTCCAT | 121 | 55 | 161 | 77.7 |
| 10694 | AGTTCAACSACACATCCTSCCATTC | 122 | 71.7 | 129 | 96.7 |
| 10695 | ACACATCSATCCATCSATCCATT | 123 | 40.6 | 108 | 40.9 |
| 7908 | TTCAACCACACATCCTTCCATTC | 124 | 9.97 | 143 | 48.5 |
| 9681 | GAGGAGGSGACCCTGSATCTGGA | 124 | 33.6 | 117 | 67.2 |
| 7169 | GGTAAAGGGAATGGCATGGCGGG | 125 | 50.2 | 110 | 68.5 |
| 10696 | AGTTCAASCACACATSCTTCCAT | 125 | 81.5 | 142 | 155 |
| 10697 | CACATCCSTCCATCCSTCCATTC | 128 | 67 | 89.1 | 32 |
| 9677 | AGGTAAASGGAATGGSATGGCGG | 128 | 77.5 | 149 | 85.4 |
| 9663 | GGGCAGSCAGGAATGGTGSGTGGAA | 129 | 72.2 | 105 | 58.4 |
| 9689 | GATCCGCSACTTAATSCACCTAC | 136 | 111 | 152 | 102 |
| 8128 | GTAATTCAACCACACATCCATCC | 137 | 54 | 107 | 52.1 |
| 9680 | AGAGGAGSTGACCCTSAATCTGG | 139 | 55.6 | 118 | 48.1 |
| 10698 | GTTCAACSACACATCSTTCCATT | 145 | 80.5 | 138 | 84.8 |
| 8130 | CAGTAATTCAACCACACATCCAT | 146 | 47.3 | 119 | 22.7 |
| 9690 | ATCCGCASCTTAATCSACCTACC | 147 | 63.2 | 132 | 76.5 |
| 7170 | AGGTAAAGGGAATGGCATGGCGG | 147 | 82.1 | 148 | 92.8 |
| 9688 | TCCGCAASTTAATCCSCCTACCC | 148 | 97 | 180 | 93.3 |
| 9679 | AGGAGGTSACCCTGASTCTGGAC | 149 | 47.3 | 101 | 58.2 |
| 10699 | AGTTCASCCACACATCCTSCCATTC | 149 | 79 | 132 | 60.8 |
| 10700 | CAGTAASTCAACCACACASCCATCC | 149 | 84.3 | 116 | 53.3 |
| 5382 | AGTTCAACCACACATCCTTCCATTC | 151 | 106 | 137 | 114 |
| 9678 | GGTAAAGSGAATGGCSTGGCGGG | 151 | 111 | 106 | 63.6 |
| 5602 | CAGTAATTCAACCACACATCCATCC | 155 | 85.5 | 133 | 58.7 |
| 10701 | TTCAACCSCACATCCSTCCATTC | 156 | 159 | 139 | 80.1 |
| 10702 | CTTGCAATSGTTCAACCASACATCC | 157 | 99.7 | 123 | 77 |
| 4572 | GAAGAGCTGGGCAGGCAGGAATGGT | 158 | 99.8 | 160 | 79.5 |
| 5549 | GATCCGCAACTTAATCCACCTACCC | 161 | 73 | 154 | 72 |
| 10703 | ACACATCCSTCCATCCATSCATTCA | 163 | 45.7 | 163 | 44.5 |
| 4733 | TGAGTAGTGAGAGTCAACCTGGAGG | 164 | 54.7 | 108 | 59.6 |
| 4631 | ATGGCATGGCGGGCAGTCTCAGAGG | 168 | 99.2 | 166 | 76.5 |
| 8129 | AGTAATTCAACCACACATCCATC | 173 | 58.2 | 107 | 51.4 |
| 5452 | TCCACCAGCCACTCACAACCATCCA | 173 | 134 | 84.3 | 45.9 |
| 7218 | AGGAGGTGACCCTGAATCTGGAC | 176 | 46.2 | 85.1 | 44.1 |
| 4984 | CACGAATCTACCCACCAACTCATCC | 178 | 116 | 140 | 49.4 |
| 7219 | GAGGAGGTGACCCTGAATCTGGA | 182 | 60.1 | 108 | 36.8 |
| 10704 | CCTTTCASTAATTCASCCACACA | 182 | 192 | 114 | 61.7 |
| 4596 | TGTGGATGGTGTGGCCAGAAAGAGG | 185 | 70.1 | 156 | 70.7 |
| 8076 | ATCCGCAACTTAATCCACCTACC | 186 | 58.2 | 149 | 63.5 |
| 9676 | GTAAAGGSAATGGCASGGCGGGC | 193 | 116 | 161 | 79.1 |
| 5460 | TTCGTCTGTCCACCAGCCACTCACA | 193 | 125 | 119 | 81.2 |
| 5467 | TCCATTTTTCGTCTGTCCACCAGCC | 196 | 122 | 113 | 51.4 |
| 7220 | AGAGGAGGTGACCCTGAATCTGG | 197 | 67.6 | 108 | 41.2 |
| 4692 | AGAGGAGGTGACCCTGAATCTGGAC | 197 | 92.2 | 119 | 37.6 |
| 4607 | GGCCAGGAGAGTGTGGATGGTGTGG | 200 | 74.8 | 162 | 83.3 |
| 7168 | GTAAAGGGAATGGCATGGCGGGC | 205 | 79.8 | 80 | 52 |
| 4645 | TCCAGGTAAAGGGAATGGCATGGCG | 215 | 102 | 149 | 127 |
| 4638 | AAAGGGAATGGCATGGCGGGCAGTC | 226 | 99.9 | 178 | 113 |
| 4807 | GGAAGAGACATACCCAGACACAAAC | 0.02 | 0.00 | 11.43 | 6.56 |
| 4812 | CAGCTGGAAGAGACATACCCAGACA | 0.03 | 0.02 | 16.20 | 7.83 |
| 4806 | GAAGAGACATACCCAGACACAAACG | 0.94 | 1.59 | 0.61 | 1.01 |
| 4836 | AGAGTTCTTTCCAGGAAACCCAGGC | 1.47 | 2.28 | 19.61 | 9.44 |
| 4837 | AAGAGTTCTTTCCAGGAAACCCAGG | 3.01 | 2.16 | 21.95 | 5.43 |
| 4835 | GAGTTCTTTCCAGGAAACCCAGGCA | 3.49 | 2.07 | 29.80 | 2.76 |
| 4834 | AGTTCTTTCCAGGAAACCCAGGCAG | 3.62 | 3.79 | 38.61 | 18.71 |
| 4815 | AGGCAGCTGGAAGAGACATACCCAG | 4.63 | 5.37 | 32.99 | 2.70 |
| 4804 | AGAGACATACCCAGACACAAACGGC | 5.98 | 6.18 | 12.51 | 6.77 |
| 4809 | CTGGAAGAGACATACCCAGACACAA | 6.26 | 10.82 | 58.24 | 12.87 |
| 5593 | AACCACACATCCATCCATCCATCCA | 41.07 | 10.10 | 97.41 | 24.31 |
| 4830 | CTTTCCAGGAAACCCAGGCAGCTGG | 42.16 | 22.26 | 77.70 | 9.85 |
| 4829 | TTTCCAGGAAACCCAGGCAGCTGGA | 51.84 | 9.47 | 72.21 | 10.14 |
| 4799 | CATACCCAGACACAAACGGCCCAAT | 60.89 | 6.26 | 84.01 | 23.09 |

*Unless otherwise noted, each of the nucleosides of antisense oligonucleotides shown in Table 36 are modified nucleosides with 2′-O-(2-methoxyethyl) (2′-MOE) sugar moieties, each “C” is replaced with a 5-methylcytosine (5-MeC), and all internucleoside linkages are phosphorothioate linkages. A spacer as indicated by S is not a nucleoside. S represents a spacer of Formula (Iia′) as disclosed herein.

### Example 13: Methods for Evaluating UNC13A Antisense Oligonucleotides

UNC13A antisense oligonucleotides were evaluated in iPSC derived human motor neurons (hMN). The cells were seeded in 96-well plates at a density of 40,000 cells/well. Antisense oligonucleotide (AON) to TDP43 was transfected with Endoporter (Gene Tools, Philomath, OR, USA) to decrease expression of the full length UNC13A transcript and increase expression of UNC13A cryptic exon. Vehicle control consisted of motor neuron treatment with Endoporter alone. Positive controls included cells that were treated with TDP43 AON alone (“AON TDP43” or “TDP43 AON”).

TDP43 AON is a gapmer oligonucleotide and has the following sequence and chemistry:

(SEQ ID NO: 1669)
5′ A*A*G*G*C*T*T*C*A*T*A*T*T*G*T*A*C*T*T*T 3′

where *=phosphorothioate, underlined=DNA, other=2′-MOE RNA; each “C” is 5-MeC.

To evaluate UNC13A AON ability to reduce UNC13A cryptic exon levels, antisense oligonucleotides to UNC13A were co-incubated with TDP43 AON in Endoporter in media before addition to the cells. After 72 hours, antisense oligonucleotides and Endoporter were washed out and replaced with fresh media alone. After six additional days, RNA was collected from the 96-well plates for RT-qPCR. RNA was isolated, cDNA generated and multiplexed RT-qPCR assay performed with Taqman probes for UNC13A cryptic exon, and reference GAPDH quantification.

Transcript levels (e.g., UNC13A cryptic exon, and TDP43 transcript) were detected by RT-qPCR using Taqman. Specifically, RT-qPCR was performed for detecting GAPDH using Thermofisher® TaqMan Gene Expression Assay Hs03929097_g1. UNC13a cryptic exon was detected using custom sequences.

UNC13a Cryptic Exon:
Forward Primer:
(SEQ ID NO: 10780)
ATTGTTCTGCACGTCGGT
Reverse Primer:
(SEQ ID NO: 10781)
GTCTGGGTATGTCTCTTCCAG
Probe Sequence:
(SEQ ID NO: 10782)
AGTTCTTTCCAGGAAACCCAGGCA

To evaluate UNC13A AON ability to reduce UNC13A correctly spliced (CS) exon junction 20/21 levels, antisense oligonucleotides to UNC13A were co-incubated with TDP43 AON in Endoporter in media before addition to the cells. After 72 hours, antisense oligonucleotides and Endoporter were washed out and replaced with fresh media alone. After six additional days, RNA was collected from the 96-well plates for RT-qPCR. RNA was isolated, cDNA generated and multiplexed RT-qPCR assay performed with Taqman probes for UNC13A CS exon 20/21 junction, and reference GAPDH quantification.

Transcript levels (e.g., UNC13A exon junction 20/21, and TDP43 transcript) were detected by RT-qPCR using Taqman. Specifically, RT-qPCR was performed for detecting GAPDH using Thermofisher® TaqMan Gene Expression Assay Hs03929097_g1. UNC13a exon 20/21 junction was detected using TaqMan Gene Expression Assay Hs01000584_m1.

RT-qPCR was performed on Applied Biosystems® 7500 Real-time PCR systems. One cycle of reverse transcription was performed at a temperature of 50° C. for 5 min. One cycle of RT inactivation/initial denaturation was performed at a temperature of 95° C. for 20 seconds. Forty five cycles of amplification were performed at a temperature of 95° C. for 1 second followed by 60° C. for 20 seconds.

UNC13A-cryptic (Ct) was normalized to GAPDH (deltaCt). To visualize the quantitative changes (e.g., % decrease of UNC13A-cryptic), the normalized UNC13A-cryptic signal was further normalized to the vehicle (treated with Endoporter alone, deltadeltaCt). Relative quantity (RQ) of transcript level was calculated using the equation RQ=2^{-deltadeltaCt} and is used to describe the treatment condition comparison to normal, healthy levels (1.0). RQ values for UNC13A cryptic were normalized using the following formula:

$$( ( ( RQAON - RQendo ) / ( RQTDP 43 - RQendo ) ) * 100$$

Table 37 shows the RT-qPCR results of UNC13A AONs with sparces and performance of UNC13A AONs without spacers in human motor neurons.

As shown in Tables 37A and 37B, UNC13A AONs (e.g., UNC13A oligonucleotides without spacers or with one or two spacers) were tested for their ability to reduce UNC13A transcripts with a cryptic exon. In some cases, UNC13A AONs with spacers reduced UNC13A cryptic exon levels. In some cases, UNC13A AONs without spacers reduced UNC13A cryptic exon levels. Specific AON sequences are labeled according to their corresponding SEQ ID NO.

Correctly spliced UNC13A (Ct) was also normalized to GAPDH (deltaCt). To visualize the quantitative changes (e.g., % increase of correctly spliced UNC13A), the normalized correctly spliced UNC13A signal was further normalized to the vehicle (treated with Endoporter alone, deltadeltaCt).

Relative quantity (RQ) of transcript level was calculated using the equation RQ=2\hat{ }(−deltadeltaCt) and is used to describe the treatment condition comparison to normal, healthy levels (1.0). RQ values for UNC13A corrected splicing were normalized using the following formula:

$$( ( ( RQAON - RQTDP 43 ) / ( R Q e n d o - R Q T D P 43 ) ) * 100$$

TABLE 37A
Exemplary UNC13A AONs evaluated in human derived iPSC motor neurons

| SEQ ID NO: | Oligonucleotide Sequence*(5′ to 3′) |
|---|---|
| 10705 | GAGACAT(S1)CCCAGAC(S1)CAAACGG |
| 10706 | GACATAC(S1)CAGACAC(S1)AACGGCC |
| 10707 | AGACATA(S1)CCAGACA(S1)AAACGGC |
| 10708 | GAGACAT(S#)CCCAGAC(S#)CAAACGG |
| 10709 | GACATAC(S#)CAGACAC(S#)AACGGCC |
| 10710 | AGACATA(S#)CCAGACA(S#)AAACGGC |
| 10711 | AGACATASCCAGACASAAACGGC |
| 10712 | GACATACSCAGACACSAACGGCC |
| 10713 | GAGACATSCCCAGACSCAAACGG |
| 10714 | GAGACATASCCAGACACASACGGCC |
| 10715 | GAGACASACCCAGACACASACGGCC |
| 7330 | AGACATACCCAGACACAAACGGC |
| 7329 | GACATACCCAGACACAAACGGCC |
| 7331 | GAGACATACCCAGACACAAACGG |
| 10716 | AAGAGACSTACCCAGSCACAAAC |
| 10717 | AGAGACASACCCAGASACAAACG |
| 10718 | GAAGAGASATACCCASACACAAA |
| 10719 | GAAGAGACSTACCCAGACSCAAACG |
| 10720 | GAAGAGSCATACCCAGACSCAAACG |
| 7333 | AAGAGACATACCCAGACACAAAC |
| 7332 | AGAGACATACCCAGACACAAACG |
| 7334 | GAAGAGACATACCCAGACACAAA |
| 4806 | GAAGAGACATACCCAGACACAAACG |
| 10721 | CAGCTGGSAGAGACASACCCAGA |
| 10722 | AGCTGGASGAGACATSCCCAGAC |
| 10723 | GCAGCTGSAAGAGACSTACCCAG |
| 7340 | CAGCTGGAAGAGACATACCCAGA |
| 7339 | AGCTGGAAGAGACATACCCAGAC |
| 7341 | GCAGCTGGAAGAGACATACCCAG |
| 10724 | GGCAGCTSGAAGAGASATACCCA |
| 10725 | AGGCAGCSGGAAGAGSCATACCC |
| 10726 | AGGCAGCTSGAAGAGACASACCCAG |
| 10727 | AGGCAGSTGGAAGAGACASACCCAG |
| 10723 | GCAGCTGSAAGAGACSTACCCAG |
| 7342 | GGCAGCTGGAAGAGACATACCCA |
| 7343 | AGGCAGCTGGAAGAGACATACCC |
| 7341 | GCAGCTGGAAGAGACATACCCAG |
| 4815 | AGGCAGCTGGAAGAGACATACCCAG |
| 10724 | TCTTTCC(S1)GGAAACC(S1)AGGCAGC |
| 10725 | GTTCTTT(S1)CAGGAAA(S1)CCAGGCA |
| 10726 | TTCTTTC(S1)AGGAAAC(S1)CAGGCAG |
| 10727 | TCTTTCC(S#)GGAAACC(S#)AGGCAGC |
| 10728 | GTTCTTT(S#)CAGGAAA(S#)CCAGGCA |
| 10729 | TTCTTTC(S#)AGGAAAC(S#)CAGGCAG |
| 10730 | GTTCTTTCSAGGAAACCCSGGCAGC |
| 10731 | GTTCTTSCCAGGAAACCCSGGCAGC |
| 10732 | TCTTTCCAGGAAACC |
| 10733 | CAGGAAACCCAGGCA |
| 10734 | TCTTTCCAGGAAACCC |
| 10735 | CCAGGAAACCCAGGCA |
| 10736 | TCTTTCCAGGAAACCCA |
| 10737 | TCCAGGAAACCCAGGCA |
| 10738 | TCTTTCCAGGAAACCCAG |
| 10739 | TTCCAGGAAACCCAGGCA |
| 10740 | TCTTTCCAGGAAACCCAGG |
| 10741 | TTTCCAGGAAACCCAGGCA |
| 10742 | TCTTTCCAGGAAACCCAGGC |
| 10743 | CTTTCCAGGAAACCCAGGCA |
| 10744 | GAGTTCTSTCCAGGASACCCAGG |
| 10755 | AGTTCTTSCCAGGAASCCCAGGC |
| 10756 | AGAGTTCSTTCCAGGSAACCCAG |
| 10757 | AGAGTTCTSTCCAGGAAASCCAGGC |
| 10758 | AGAGTTSTTTCCAGGAAASCCAGGC |
| 7363 | GAGTTCTTTCCAGGAAACCCAGG |
| 7362 | AGTTCTTTCCAGGAAACCCAGGC |
| 7364 | AGAGTTCTTTCCAGGAAACCCAG |
| 4836 | AGAGTTCTTTCCAGGAAACCCAGGC |
| 10759 | CACATCC(S1)TCCATCC(S1)TCCATTC |
| 10760 | ACACATC(S1)ATCCATC(S1)ATCCATT |
| 10761 | ACATCCA(S1)CCATCCA(S1)CCATTCA |
| 10762 | CACATCC(S#)TCCATCC(S#)TCCATTC |
| 10763 | ACACATC(S#)ATCCATC(S#)ATCCATT |
| 10764 | ACATCCA(S#)CCATCCA(S#)CCATTCA |
| 10765 | GCAGCTS[LNA-G]AAGAGACATAS[LNA-C]CAGAC |
| 10766 | GCAGC[LNA-T]SGAAGAGACATAS[LNA-C]CAGAC |
| 10767 | GCAGC[LNA-T]SGAAGAGACAT[LNA-A]SCCAGAC |
| 10768 | GCA[LNA-G]CTSGAAGA[LNA-G]ACATASCC[LNA-A]GAC |
| 10769 | GCA[LNA-G]CTSGAAGAGACATASCC[LNA-A]GAC |
| 10770 | GCAGCTGGS[LNA-A]GAGACATAS[LNA-C]CAGAC |
| 10771 | GCAGCTG[LNA-G]SAGAGACATAS[LNA-C]CAGAC |
| 10772 | GCAGCTG[LNA-G]SAGAGACAT[LNA-A]SCCAGAC |
| 10773 | GCA[LNA-G]CTGGSAGA[LNA-G]ACATASCC[LNA-A]GAC |
| 10774 | GCA[LNA-G]CTGGSAGAGACATASCC[LNA-A]GAC |
| 10775 | GTTCTTTS[LNA-C]AGGAAAS[LNA-C]CAGGCA |
| 10776 | GTTCTT\|LNA-T\|SCAGGAAASILNA-C]CAGGCA |
| 10777 | GTTCTT[LNA-T]SCAGGAA[LNA-A]SCCAGGCA |
| 10778 | GTT[LNA-C]TTTSCAG[LNA-G]AAASCCA[LNA-G]GCA |
| 10779 | GTT[LNA-C]TTTSCAGGAAASCCA[LNA-G]GCA |

*Unless otherwise noted, each of the nucleosides of antisense oligonucleotides shown in Table 37A are modified nucleosides with 2′-O-(2-methoxyethyl) (2′-MOE) sugar moieties, each “C” is replaced with a 5-methylcytosine (5-MeC), and all internucleoside linkages are phosphorothioate linkages. A spacer, as indicated by any of S, S1, or S#, is not a nucleoside. S represents a spacer of Formula (Iia′) as disclosed herein. S1 represents a spacer of Formula (IIIa′); S# represents a spacer of Formula (Ilib′). [LNA-X] refers to a locked nucleic acid with a corresponding base X (e.g., LNA-A refers to a locked nucleic acid with an adenine nucleobase, LNA-G refers to a locked nucleic acid with a guanine nucleobase, LNA-T refers to a locked nucleic acid with a thymine nucleobase, and LNA-C refers to a locked nucleic acid with a 5-methylcytosine nucleobase).

TABLE 37B
Performance of Exemplary UNC13A AONs evaluated in human derived iPSC motor neurons

| | | UNC13A Corrected Splicing | | | | UNC13A Cryptic Splicing | | | |
|---|---|---|---|---|---|---|---|---|---|
| | | Normalized Rq | | | | Normalized Rq | | | |
| SEQ ID | | 200 nM | | 50 nM | | 200 nM | | 50 nM | |
| NO: | Oligonucleotide Sequence* (5′ to 3′) | Mean | STD | Mean | STD | Mean | STD | Mean | STD |
| 4806 | GAAGAGACATACCCAGACACAAACG | 41.82 | 18.84 | −27.56 | 73.08 | 3.16 | 1.03 | 18.87 | 12.38 |
| 7334 | GAAGAGACATACCCAGACACAAA | 8.93 | 7.32 | −7.13 | 7.81 | 15.78 | 7.41 | 42.31 | 31.11 |
| 7333 | AAGAGACATACCCAGACACAAAC | −28.45 | 72.83 | −12.62 | 12.84 | 12.06 | 9.62 | 57.27 | 34.91 |
| 7332 | AGAGACATACCCAGACACAAACG | 13.03 | 15.19 | 9.48 | 11.41 | 14.78 | 6.73 | 61.44 | 25.31 |
| 7331 | GAGACATACCCAGACACAAACGG | 28.03 | 29.67 | 7.69 | 35.17 | 18.53 | 10.52 | 33.25 | 20.41 |
| 7330 | AGACATACCCAGACACAAACGGC | 24.21 | 33.06 | 23.24 | 30.63 | 29.74 | 33.32 | 50.76 | 39.49 |
| 7329 | GACATACCCAGACACAAACGGCC | 2.47 | 35.11 | 2.89 | 26.90 | 26.29 | 25.67 | 46.66 | 32.30 |
| 4815 | AGGCAGCTGGAAGAGACATACCCAG | 47.19 | 57.85 | 15.52 | 30.16 | 2.07 | 3.30 | 13.45 | 13.12 |
| 7343 | AGGCAGCTGGAAGAGACATACCC | 37.80 | 13.72 | 1.51 | 9.33 | 2.32 | 3.41 | 22.31 | 14.52 |
| 7342 | GGCAGCTGGAAGAGACATACCCA | 39.83 | 25.50 | 19.20 | 21.74 | 7.01 | 5.73 | 25.13 | 28.23 |
| 7341 | GCAGCTGGAAGAGACATACCCAG | 43.08 | 23.32 | 4.48 | 7.87 | 5.11 | 6.91 | 32.17 | 23.76 |
| 7340 | CAGCTGGAAGAGACATACCCAGA | −21.34 | 16.48 | −144.30 | 28.86 | 7.59 | 6.87 | 21.20 | 16.74 |
| 7339 | AGCTGGAAGAGACATACCCAGAC | −173.70 | 29.80 | −75.73 | 8.72 | 4.20 | 7.60 | 165.20 | 96.22 |
| 10723 | GCAGCTGSAAGAGACSTACCCAG | 64.46 | 20.00 | −11.79 | 20.17 | 2.34 | 1.77 | 17.01 | 6.37 |
| 10721 | CAGCTGGSAGAGACASACCCAGA | 87.01 | 8.70 | 28.35 | 13.19 | 5.72 | 3.41 | 22.86 | 10.45 |
| 10731 | GTTCTTSCCAGGAAACCCSGGCAGC | −8.32 | 3.39 | −10.50 | 8.45 | 29.49 | 14.34 | 51.95 | 12.36 |
| 10730 | GTTCTTTCSAGGAAACCCSGGCAGC | 6.29 | 22.23 | 6.58 | 3.64 | 8.10 | 5.42 | 28.87 | 7.85 |
| 10756 | AGAGTTCSTTCCAGGSAACCCAG | 61.99 | 21.81 | −24.46 | 58.00 | 12.82 | 3.63 | 55.05 | 18.66 |
| 10744 | GAGTTCTSTCCAGGASACCCAGG | 38.47 | 29.10 | −14.70 | 48.97 | 17.11 | 11.96 | 80.63 | 36.03 |
| 10755 | AGTTCTTSCCAGGAASCCCAGGC | 9.90 | 25.86 | −7.31 | 26.47 | 26.33 | 9.18 | 92.35 | 32.38 |
| 10758 | AGAGTTSTTTCCAGGAAASCCAGGC | 44.52 | 32.61 | 57.40 | 14.54 | 9.44 | 4.83 | 22.79 | 6.99 |
| 10757 | AGAGTTCTSTCCAGGAAASCCAGGC | 44.46 | 8.40 | 48.23 | 22.36 | 3.31 | 2.76 | 14.18 | 10.22 |
| 10713 | GAGACATSCCCAGACSCAAACGG | 84.50 | 29.82 | 47.59 | 10.35 | 31.74 | 13.09 | 79.61 | 17.53 |
| 10711 | AGACATASCCAGACASAAACGGC | 90.95 | 10.72 | 22.20 | 40.15 | 30.38 | 13.73 | 82.12 | 14.48 |
| 10712 | GACATACSCAGACACSAACGGCC | 73.81 | 41.97 | 36.23 | 29.75 | 22.06 | 9.58 | 70.87 | 9.37 |
| 5245 | GAGACASACCCAGACACASACGGCC | 80.40 | 19.16 | 23.13 | 19.81 | 31.05 | 15.73 | 88.26 | 28.47 |
| 5244 | GAGACATASCCAGACACASACGGCC | 126.50 | 25.60 | 88.00 | 43.30 | 17.80 | 10.45 | 77.70 | 25.98 |
| 5257 | AGGCAGSTGGAAGAGACASACCCAG | 96.50 | 22.07 | 44.00 | 25.12 | 10.88 | 6.15 | 35.95 | 13.92 |
| 5256 | AGGCAGCTSGAAGAGACASACCCAG | 103.10 | 12.62 | 66.18 | 15.49 | 4.04 | 2.80 | 27.84 | 10.66 |
| 5255 | AGGCAGCSGGAAGAGSCATACCC | 92.83 | 15.27 | 58.96 | 6.90 | 12.26 | 4.78 | 31.13 | 9.49 |
| 5254 | GGCAGCTSGAAGAGASATACCCA | 82.43 | 18.79 | 58.45 | 12.96 | 8.83 | 8.20 | 37.74 | 10.12 |
| 5252 | AGCTGGASGAGACATSCCCAGAC | 87.57 | 8.14 | 9.59 | 32.49 | 7.43 | 3.22 | 28.79 | 15.38 |
| 5250 | GAAGAGSCATACCCAGACSCAAACG | 102.70 | 14.83 | 54.28 | 31.30 | 29.53 | 14.04 | 75.86 | 28.78 |
| 5249 | GAAGAGACSTACCCAGACSCAAACG | 99.18 | 24.63 | 46.29 | 25.42 | 22.36 | 7.32 | 75.43 | 35.92 |
| 5248 | GAAGAGASATACCCASACACAAA | 49.24 | 20.69 | 21.53 | 23.44 | 89.72 | 39.56 | 135.70 | 43.02 |
| 5246 | AAGAGACSTACCCAGSCACAAAC | 28.09 | 21.14 | 22.72 | 15.94 | 52.17 | 11.17 | 112.70 | 38.44 |
| 5247 | AGAGACASACCCAGASACAAACG | 78.70 | 27.23 | 29.31 | 21.53 | 29.70 | 17.53 | 85.44 | 19.01 |
| 5292 | GTT[LNA-C]TTTSCAGGAAASCCA[LNA-G]GCA | 25.91 | 16.04 | 21.89 | 19.68 | 4.99 | 12.69 | 17.06 | 44.28 |
| 5291 | GTT[LNA-C]TTTSCAG[LNA-G]AAASCCA[LNA-G]GCA | 19.94 | 10.97 | 10.81 | 5.00 | 20.25 | 27.06 | 28.08 | 34.90 |
| 5290 | GTTCTT[LNA-T]SCAGGAA[LNA-A]SCCAGGCA | 8.93 | 9.25 | 6.86 | 7.65 | −1.28 | 1.43 | 7.60 | 13.16 |
| 5289 | GTTCTT[LNA-T]SCAGGAAAS[LNA-C]CAGGCA | 0.18 | 4.96 | 12.23 | 11.77 | −1.20 | 1.39 | 29.03 | 35.58 |
| 5288 | GTTCTTTS[LNA-C]AGGAAAS[LNA-C]CAGGCA | 14.39 | 10.91 | 10.40 | 7.95 | 6.29 | 12.97 | 21.83 | 31.86 |
| 5287 | GCA[LNA-G]CTGGSAGAGACATASCC[LNA-A]GAC | −10.22 | 30.13 | 29.91 | 20.97 | 11.14 | 21.19 | 6.45 | 7.80 |
| 5286 | GCA[LNA-G]CTGGSAGA[LNA-G]ACATASCC[LNA-A]GAC | 10.40 | 16.54 | 5.17 | 18.78 | 12.36 | 10.45 | 32.23 | 21.90 |
| 5285 | GCAGCTG[LNA-G]SAGAGACAT[LNA-A]SCCAGAC | 0.57 | 12.49 | −14.07 | 16.60 | 7.76 | 8.69 | 16.24 | 14.15 |
| 5284 | GCAGCTG[LNA-G]SAGAGACATAS[LNA-C]CAGAC | −6.16 | 17.10 | 7.57 | 18.74 | 14.62 | 9.19 | 46.32 | 24.49 |
| 5283 | GCAGCTGGS[LNA-A]GAGACATAS[LNA-C]CAGAC | 18.14 | 16.98 | 9.28 | 26.20 | 8.90 | 6.17 | 40.24 | 12.47 |
| 5292 | GTT[LNA-C]TTTSCAGGAAASCCA[LNA-G]GCA | 4.16 | 16.50 | 4.89 | 11.13 | 15.39 | 21.60 | 19.66 | 21.58 |
| 5291 | GTT[LNA-C]TTTSCAG[LNA-G]AAASCCA[LNA-G]GCA | −0.74 | 10.05 | 1.42 | 8.15 | 11.70 | 16.06 | 52.88 | 33.79 |
| 5290 | GTTCTT[LNA-T]SCAGGAA[LNA-A]SCCAGGCA | −3.14 | 10.20 | 0.93 | 12.63 | 11.03 | 12.72 | 21.48 | 26.72 |
| 5289 | GTTCTT[LNA-T]SCAGGAAAS[LNA-C]CAGGCA | 0.43 | 5.96 | 0.99 | 9.62 | 6.88 | 15.65 | 39.99 | 36.29 |
| 5288 | GTTCTTTS[LNA-C]AGGAAAS[LNA-C]CAGGCA | −6.06 | 6.61 | −11.46 | 10.35 | 6.58 | 11.97 | 20.12 | 30.75 |
| 10711 | AGACATASCCAGACASAAACGGC | 37.10 | 22.45 | 53.64 | 15.42 | 43.13 | 22.30 | 79.66 | 21.62 |
| 10712 | GACATACSCAGACACSAACGGCC | 50.01 | 21.60 | 21.58 | 15.74 | 67.02 | 35.41 | 83.55 | 61.27 |
| 10713 | GAGACATSCCCAGACSCAAACGG | 49.95 | 11.51 | 29.51 | 6.61 | 40.47 | 25.79 | 143.30 | 44.64 |
| 10707 | AGACATA(S1)CCAGACA(S1)AAACGGC | 41.00 | 21.58 | 27.91 | 8.85 | 51.69 | 49.87 | 103.79 | 37.66 |
| 10706 | GACATAC(S1)CAGACAC(S1)AACGGCC | 46.48 | 5.54 | 20.34 | 11.08 | 9.03 | 14.52 | 72.91 | 57.87 |
| 10705 | GAGACAT(S1)CCCAGAC(S1)CAAACGG | 45.33 | 8.89 | 33.99 | 13.59 | 23.89 | 18.73 | 133.34 | 42.50 |
| 9687 | TTCTTTCSAGGAAACSCAGGCAG | 11.16 | 8.04 | 9.91 | 7.60 | 5.90 | 10.52 | 94.03 | 74.88 |
| 9686 | GTTCTTTSCAGGAAASCCAGGCA | 5.10 | 4.93 | −8.11 | 6.11 | 12.92 | 15.28 | 25.15 | 25.51 |
| 9685 | TCTTTCCSGGAAACCSAGGCAGC | 10.06 | 10.19 | 3.12 | 11.72 | 6.96 | 9.36 | 26.97 | 9.87 |
| 10726 | TTCTTTC(S1)AGGAAAC(S1)CAGGCAG | 11.38 | 11.69 | 7.69 | 15.12 | 11.99 | 14.32 | 54.16 | 41.27 |
| 10725 | GTTCTTT(S1)CAGGAAA(S1)CCAGGCA | 1.44 | 7.33 | −4.17 | 4.75 | 9.79 | 9.57 | 27.69 | 15.98 |
| 10724 | TCTTTCC(S1)GGAAACC(S1)AGGCAGC | 7.18 | 11.81 | −11.24 | 5.27 | 18.13 | 13.56 | 55.71 | 28.06 |
| 10692 | ACATCCASCCATCCASCCATTCA | −37.97 | 26.85 | −4.07 | 52.32 | 129.74 | 41.79 | 122.30 | 48.42 |
| 10695 | ACACATCSATCCATCSATCCATT | 5.77 | 35.61 | −11.91 | 27.75 | 96.81 | 38.14 | 111.73 | 35.15 |
| 10697 | CACATCCSTCCATCCSTCCATTC | −14.00 | 37.66 | 1.96 | 30.50 | 159.36 | 43.66 | 164.55 | 47.07 |
| 10761 | ACATCCA(S1)CCATCCA(S1)CCATTCA | −16.66 | 14.80 | −5.26 | 26.53 | 165.82 | 42.96 | 151.38 | 29.35 |
| 10760 | ACACATC(S1)ATCCATC(S1)ATCCATT | −7.44 | 24.85 | −21.89 | 16.44 | 162.48 | 39.49 | 116.38 | 15.71 |
| 10759 | CACATCC(S1)TCCATCC(S1)TCCATTC | −9.65 | 36.42 | −0.61 | 27.96 | 145.25 | 19.25 | 132.10 | 35.74 |
| 10814 | TCTTTCCAGGAAACCCAGGCA | 18.29 | 17.99 | 9.91 | 14.01 | 16.42 | 18.29 | 16.34 | 9.91 |
| 10733 | CAGGAAACCCAGGCA | 2.74 | 3.88 | 2.44 | 1.85 | 16.24 | 2.74 | 14.66 | 2.44 |
| 10732 | TCTTTCCAGGAAACC | 12.94 | 2.00 | 7.34 | 7.10 | 12.75 | 12.94 | 12.29 | 7.34 |
| 10735 | CCAGGAAACCCAGGCA | 0.00 | 0.00 | 0.74 | 1.05 | 13.49 | 0.00 | 16.04 | 0.74 |
| 10734 | TCTTTCCAGGAAACCC | 17.89 | 25.30 | 17.60 | 2.62 | 18.82 | 17.89 | 15.79 | 17.60 |
| 10737 | TCCAGGAAACCCAGGCA | 0.00 | 0.00 | 1.54 | 2.17 | 14.54 | 0.00 | 15.41 | 1.54 |
| 10736 | TCTTTCCAGGAAACCCA | 6.80 | 9.61 | 2.79 | 1.25 | 12.77 | 6.80 | 15.04 | 2.79 |
| 10739 | TTCCAGGAAACCCAGGCA | 0.70 | 0.98 | 0.00 | 0.00 | 16.31 | 0.70 | 11.42 | 0.00 |
| 10738 | TCTTTCCAGGAAACCCAG | 19.21 | 8.43 | 49.84 | 70.94 | 16.89 | 19.21 | 19.14 | 49.84 |
| 10741 | TTTCCAGGAAACCCAGGCA | 0.55 | 0.78 | 3.96 | 5.60 | 13.97 | 0.55 | 12.15 | 3.96 |
| 10740 | TCTTTCCAGGAAACCCAGG | 11.47 | 8.56 | 2.24 | 3.17 | 16.52 | 11.47 | 15.47 | 2.24 |
| 10743 | CTTTCCAGGAAACCCAGGCA | 6.60 | 0.75 | 1.59 | 2.25 | 18.60 | 6.60 | 12.55 | 1.59 |
| 10742 | TCTTTCCAGGAAACCCAGGC | 15.08 | 15.12 | 11.29 | 4.48 | 15.92 | 15.08 | 12.81 | 11.29 |

*Unless otherwise noted, each of the nucleosides of antisense oligonucleotides shown in Table 37B are modified nucleosides with 2′-O-(2-methoxyethyl) (2′-MOE) sugar moieties, each ″C″ is replaced with a 5-methylcytosine (5-MeC), and all internucleoside linkages are
phosphorothioate linkages. A spacer, as indicated by any of S, S1, or S#, is not a nucleoside. S represents a spacer of Formula (Iia′) as disclosed herein. S1 represents a spacer of Formula (IIIa′); S# represents a spacer of Formula (Ilib′). [LNA-X] refers to a locked nucleic acid with a corresponding base X (e.g., LNA-A refers to a locked nucleic acid with an adenine nucleobase, LNA-G refers to a locked nucleic acid with a guanine nucleobase, LNA-T refers to a locked nucleic acid with a thymine nucleobase, and LNA-C refers to a locked nucleic acid with a 5-methylcytosine nucleobase).

### Example 14: Design and Selection of SMN2 Oligonucleotides

SMN2 AONs oligonucleotides that target a SMN2 transcript are designed and tested to identify SMN2 AONs capable of reducing quantity of mis-spliced SMN2 transcripts. Such SMN2 AONs include a subset of the SMN2 oligonucleotide variants represented by any of SEQ ID NOs: 9710-10141. The SMN2 parent oligonucleotides are 25 nucleosides in length. Each of the nucleosides of the SMN2 parent oligonucleotides are modified nucleosides with 2′-MOE sugar moieties, and each “C” is replaced with a 5-MeC. Additionally, each of the internucleoside linkages between the nucleosides of the SMN2 oligonucleotides are phosphorothioate internucleoside linkages.

Generally, the length of the SMN2 antisense oligonucleotides are 25 oligonucleotide units in length. However, variants of the SMN2 antisense oligonucleotides were also designed with varying lengths (e.g., 23mers). Examples of these variant SMN2 antisense oligonucleotides were designed to include a subset of the sequences of SEQ ID NOs: 9710-10141.

TABLE 38
SMN2 AONs (including SMN2 parent
oligonucleotides and SMN2
oligonucleotides with two spacers)

| | Oligonucleotide Sequence* |
|---|---|
| | (where S indicates |
| SEQ | presence of a Spacer) |
| ID No: | (5′ → 3′) |
| 10641 | ATTCACTTTCATAATGCTGG |
| 10642 | GTAAGATTCACTTTCATAATGCTGG |
| 10643 | ATTCACTTTCATAATGCTGGCAGAC |
| 10644 | GTAAGASTCACTTTCATASTGCTGG |
| 10645 | GTAAGATTSACTTTCATASTGCTGG |
| 10646 | ATTCACSTTCATAATGCTSGCAGAC |
| 10647 | ATTCACTTSCATAATGCTSGCAGAC |
| 10648 | AAGATTCACTTTCATAATGCTGG |
| 10649 | AAGATTCSCTTTCATSATGCTGG |
| 10650 | AGATTCACTTTCATAATGCTGGC |
| 10651 | AGATTCASTTTCATASTGCTGGC |
| 9971 | GATTCACTTTCATAATGCTGGCA |
| 10783 | GATTCACSTTCATAASGCTGGCA |
| 9970 | ATTCACTTTCATAATGCTGGCAG |
| 10784 | ATTCACTSTCATAATSCTGGCAG |

### Example 15: Methods for Evaluating SMN2 Antisense Oligonucleotides

SMN2 antisense oligonucleotides were evaluated in primary human fibroblasts from an SMA patient (NIGMS Human Genetic Cell Repository, Coriell Institute, Camden, New Jersey). The cells were seeded in 96-well plates at a density of 20,000 cells/well. Antisense oligonucleotide (AON) to SMN2 was transfected into fibroblasts with Endoporter (Gene Tools, Philomath, OR, USA) to increase expression of the full length SMN2 transcript and reduce expression of the Delta7 variant. Vehicle control consisted of cell treatment with Endoporter alone. Positive controls included cells that were treated with AON control alone.

AON control is a splice switching oligonucleotide and has the following sequence and chemistry:

(SEQ ID NO: 9697)
5′ A*T*T*C*A*C*T*T*T*C*A*T*A*A*T*G*C*T*G*G 3′

where *=phosphorothioate, underlined=DNA, other=2′-MOE RNA; each “C” is 5-MeC.

To evaluate SMN2 AON ability to restore SMN2-FL and reduce the Delta7 transcript, antisense oligonucleotides to SMN2 were co-incubated Endoporter in media before addition to the cells. After 72 hours, antisense oligonucleotides and endoporter were washed out and replaced with fresh media. After 72 additional hours, RNA was collected from the 96-well plates for RT-qPCR. RNA was isolated, cDNA generated and multiplexed RT-qPCR assay performed with taqman probes for SMN2 full length transcript, SMN2 Delta7 transcript, and reference GAPDH quantification.

Transcript levels (e.g., SMN2 full length transcript or TDP43 transcript) were detected by RT-qPCR using Taqman. Specifically, RT-qPCR was performed for detecting GAPDH using Thermofisher TaqMan Gene Expression Assay Hs03929097_g1. RT-qPCR was performed for detecting SMN2 transcripts using custom Thermofisher TaqMan Gene Expression Assays with the following sequences:

SMN2-FL:
Forward Primer:
(SEQ ID NO: 10810)
5′ GCTCACATTCCTTAAATTAAGGAGAAA 3′
Reverse Primer:
(SEQ ID NO: 10811)
5′ TCCAGATCTGTCTGATCGTTTCTT 3′
Probe Sequence:
(SEQ ID NO: 10812)
5′ CTGGCATAGAGCAGCACTAAATGACACCAC 3′
SMN2 Delta7:
Forward Primer:
(SEQ ID NO: 10813)
5′ TGGCTATCATACTGGCTATTATATGGAA 3′
Reverse Primer:
(SEQ ID NO: 10811)
5′ TCCAGATCTGTCTGATCGTTTCTT 3′
Probe Sequence:
(SEQ ID NO: 10812)
5′ CTGGCATAGAGCAGCACTAAATGACACCAC 3′

RT-qPCR was performed on Applied Biosystems® 7500 Real-time PCR systems. One cycle of reverse transcription was performed at a temperature of 50° C. for 5 min. One cycle of RT inactivation/initial denaturation was performed at a temperature of 95° C. for 20 seconds. Forty five cycles of amplification were performed at a temperature of 95° C. for 1 second followed by 60° C. for 20 seconds.

2-FL and SMN2 Delta7 (Ct) was normalized to GAPDH (deltaCt). To visualize the quantitative changes (e.g., % increase of SMN2-FL, decrease of SMN2 Delta7), the normalized SMN2-FL and SMN2 Delta7 signal was further normalized to the vehicle (treated with Endoporter alone, deltadeltaCt). Relative quantity of transcript level was calculated using the equation RQ=2^{-deltadeltaCt} and is used to describe the treatment condition comparison to normal, healthy levels (1.0).

TABLE 39
Performance of SMN2 AONs evaluated in human derived iPSC motor neurons

| | | 200 nM | 500 nM | 200 nM | 500 nM |
|---|---|---|---|---|---|
| | | ASO | ASO | ASO | ASO |
| SEQ | | SMN2 | SMN2 | SMN2 | SMN2 |
| ID | Oligonucleotide Sequence* | FL | FL | Delta7 | Delta7 |
| NO: | (5′ to 3′) | Rg | Rq | Rq | Rq |
| 10641 | ATTCACTTTCATAATGCTGG | 1.9895 | 1.7061 | 0.8194 | 0.4527 |
| 9971 | GATTCACTTTCATAATGCTGGCA | 1.3173 | 1.2035 | 0.5861 | 0.1667 |
| 10783 | GATTCACSTTCATAASGCTGGCA | 1.3833 | 1.4217 | 0.5407 | 0.2094 |
| 9972 | AGATTCACTTTCATAATGCTGGC | 1.3820 | 1.4660 | 0.6529 | 0.0964 |
| 10651 | AGATTCASTTTCATASTGCTGGC | 1.3113 | 1.1082 | 0.5664 | 0.2429 |
| 9970 | ATTCACTTTCATAATGCTGGCAG | 1.2012 | 0.9794 | 0.6978 | 0.1799 |
| 10784 | ATTCACTSTCATAATSCTGGCAG | 1.3275 | 0.8486 | 0.6869 | 0.3260 |
| 9973 | AAGATTCACTTTCATAATGCTGG | 1.1847 | 1.3186 | 0.9021 | 0.4403 |
| 10649 | AAGATTCSCTTTCATSATGCTGG | 0.9119 | 1.1494 | 0.8714 | 0.4857 |
| 10643 | ATTCACTTTCATAATGCTGGCAGAC | 0.8669 | 0.8240 | 1.1727 | 1.6183 |
| 10646 | ATTCACSTTCATAATGCTSGCAGAC | 0.7046 | 0.5606 | 1.2103 | 1.5860 |
| 10647 | ATTCACTTSCATAATGCTSGCAGAC | 0.7889 | 0.4940 | 1.2433 | 1.2746 |
| 10642 | GTAAGATTCACTTTCATAATGCTGG | 1.9557 | 2.4185 | 0.9007 | 0.4565 |
| 10644 | GTAAGASTCACTTTCATASTGCTGG | 1.8845 | 2.2573 | 0.9753 | 0.8837 |
| 10645 | GTAAGATTSACTTTCATASTGCTGG | 1.2126 | 1.6212 | 0.8690 | 0.6738 |

*Unless otherwise noted, each of the nucleosides of antisense oligonucleotides shown in Table 39 are modified nucleosides with 2′-O-(2-methoxyethyl) (2′-MOE) sugar moieties, each ″C″ is replaced with a 5-methylcytosine (5-MeC), and all internucleoside linkages are phosphorothioate linkages. A spacer as indicated by S is not a nucleoside. S represents a spacer of Formula (Iia′) as disclosed herein.

### Example 16: Methods for Evaluating SMN2 Antisense Oligonucleotides

SMN2 antisense oligonucleotides were evaluated in human derived iPSC motor neurons from an SMA patient (NIGMS Human Genetic Cell Repository, Coriell Institute, Camden, New Jersey). The cells were seeded in 96-well plates at a density of 40,000 cells/well. Antisense oligonucleotide (AON) to SMN2 was transfected into motor neurons with Endoporter (Gene Tools, Philomath, OR, USA) to increase expression of the full length SMN2 transcript and reduce expression of the Delta7 variant. Vehicle control consisted of cell treatment with Endoporter alone. Positive controls included cells that were treated with an AON control alone.

The AON Control is a splice switching oligonucleotide and has the following sequence and chemistry:
5′ T*C*A*C*T*T*T*C*A*T*A*A*T*G*C*T*G*G* 3′ (SEQ ID NO: 10809)
   where *=phosphorothioate, underlined=DNA, other=2′-MOE RNA; each “C” is 5-MeC.

To evaluate SMN2 AON ability to restore SMN2-FL and reduce the Delta7 transcript, antisense oligonucleotides to SMN2 were co-incubated Endoporter in media before addition to the cells. After 72 hours, antisense oligonucleotides and endoporter were washed out and replaced with fresh media. After six additional days, RNA was collected from the 96-well plates for RT-qPCR. RNA was isolated, cDNA generated and multiplexed RT-qPCR assay performed with taqman probes for SMN2 full length transcript, SMN2 Delta7 transcript, and reference GAPDH quantification.

Transcript levels (e.g., SMN2 full length transcript or SMN2 Delta7 transcript) were detected by RT-qPCR using Taqman. Specifically, RT-qPCR was performed for detecting GAPDH using Thermofisher TaqMan Gene Expression Assay Hs03929097_g1. RT-qPCR was performed for detecting SMN2 transcripts using custom Thermofisher TaqMan Gene Expression Assays with the following sequences:

SMN2-FL:
Forward Primer:
(SEQ ID NO: 10810)
5′ GCTCACATTCCTTAAATTAAGGAGAAA 3′
Reverse Primer:
(SEQ ID NO: 10811)
5′ TCCAGATCTGTCTGATCGTTTCTT 3′
Probe Sequence:
(SEQ ID NO: 10812)
5′ CTGGCATAGAGCAGCACTAAATGACACCAC 3′
SMN2 Delta7:
Forward Primer:
(SEQ ID NO: 10813)
5′ TGGCTATCATACTGGCTATTATATGGAA 3′
Reverse Primer:
(SEQ ID NO: 10811)
5′ TCCAGATCTGTCTGATCGTTTCTT 3′
Probe Sequence:
(SEQ ID NO: 10812)
5′ CTGGCATAGAGCAGCACTAAATGACACCAC 3′

RT-qPCR was performed on Applied Biosystems® 7500 Real-time PCR systems. One cycle of reverse transcription was performed at a temperature of 50° C. for 5 min. One cycle of RT inactivation/initial denaturation was performed at a temperature of 95° C. for 20 seconds. Forty five cycles of amplification were performed at a temperature of 95° C. for 1 second followed by 60° C. for 20 seconds. Relative quantity (RQ) of the transcript levels are displayed in the table.

SMN2-FL and SMN2 Delta7 (Ct) was normalized to GAPDH (deltaCt). To visualize the quantitative changes (e.g., % increase of SMN2-FL, decrease of SMN2 Delta7), the normalized SMN2-FL and SMN2 Delta7 signal was further normalized to the vehicle (treated with Endoporter alone, deltadeltaCt). Relative quantity of transcript level was calculated using the equation RQ=2^{-deltadeltaCt} and is used to describe the treatment condition comparison to normal, healthy levels (1.0).

TABLE 40
Performance of SMN2 AONs evaluated in human
derived iPSC motor neurons

| | | 200 nM | 50 nM | 200 nM | 50 nM |
|---|---|---|---|---|---|
| | | ASO | ASO | ASO | ASO |
| SEQ | | SMN2 | SMN2 | SMN2 | SMN2 |
| ID | Oligonucleotide | FL | FL | Delta7 | Delta7 |
| NO: | Sequence* (5′ to 3′) | RQ | RQ | RQ | RQ |
| 10641 | ATTCACTTTCATAATGCTGG | 2.7 | 2.99 | 0.0861 | 0.0285 |
| 10642 | GTAAGATTCACTTTCATAATGCTGG | 1.85 | 2.1 | 0.186 | 0.172 |
| 10643 | ATTCACTTTCATAATGCTGGCAGAC | 0.131 | 0.427 | 15 | 2.28 |
| 10644 | GTAAGASTCACTTTCATASTGCTGG | 2.28 | 2.36 | 0.247 | 0.139 |
| 10645 | GTAAGATTSACTTTCATASTGCTGG | 1.79 | 2.21 | 0.0916 | 0.157 |
| 10646 | ATTCACSTTCATAATGCTSGCAGAC | 0.0894 | 0.132 | 3.25 | 2.77 |
| 10647 | ATTCACTTSCATAATGCTSGCAGAC | 0.0709 | 0.142 | 2.75 | 2.98 |
| 10648 | AAGATTCACTTTCATAATGCTGG | 0.173 | 0.187 | 2.82 | 2.95 |
| 10649 | AAGATTCSCTTTCATSATGCTGG | 2.41 | 2.2 | 0.0701 | 0.212 |
| 10650 | AGATTCACTTTCATAATGCTGGC | 2.34 | 2.79 | 0.0197 | 0.0479 |
| 10651 | AGATTCASTTTCATASTGCTGGC | 2.67 | 2.97 | 0.0413 | 0.11 |
| 9971 | GATTCACTTTCATAATGCTGGCA | 2.71 | 2.75 | 0.023 | 0.0892 |
| 10783 | GATTCACSTTCATAASGCTGGCA | 3.08 | 2.94 | 0.0343 | 0.101 |
| 9970 | ATTCACTTTCATAATGCTGGCAG | 1.86 | 1.7 | 0.08 | 0.0999 |
| 10784 | ATTCACTSTCATAATSCTGGCAG | 1.88 | 2.03 | 0.224 | 0.416 |
| 10785 | TCACTTTCATAATGCTGG | 1.88 | 2.45 | 0.011 | 0.0206 |
| 10786 | TCACTTTCSTAATGCTGG | 1.75 | 2.41 | 0.012 | 0.0253 |
| 10787 | TCACTTTCASAATGCTGG | 2.40 | 2.290 | 0.007 | 0.0132 |
| 10788 | TCACTTTCSSAATGCTGG | 1.82 | 2.23 | 0.0189 | 0.0322 |

*Unless otherwise noted, each of the nucleosides of antisense oligonucleotides shown in Table 40 are modified nucleosides with 2′-O-(2-methoxyethyl) (2′-MOE) sugar moieties, each ″C″ is replaced with a 5-methylcytosine (5-MeC) , and all internucleoside linkages are phosphorothioate linkages. A spacer as indicated by S is not a nucleoside. S represents a spacer of Formula (Iia′) as disclosed herein.

### Example 17: Methods for Evaluating SMN2 Antisense Oligonucleotides with Locked Nucleic Acids

SMN2 antisense oligonucleotides were evaluated in human derived iPSC motor neurons from an SMA patient (NIGMS Human Genetic Cell Repository, Coriell Institute, Camden, New Jersey). The cells were seeded in 96-well plates at a density of 40,000 cells/well. Antisense oligonucleotide (AON) to SMN2 was transfected into motor neurons with Endoporter (Gene Tools, Philomath, OR, USA) to increase expression of the full length SMN2 transcript and reduce expression of the Delta7 variant. Vehicle control consisted of cell treatment with Endoporter alone.

To evaluate SMN2 AON ability to restore SMN2-FL and reduce the Delta7 transcript, antisense oligonucleotides to SMN2 were co-incubated Endoporter in media before addition to the cells. After 72 hours, antisense oligonucleotides and endoporter were washed out and replaced with fresh media. After six additional days, RNA was collected from the 96-well plates for RT-qPCR. RNA was isolated, cDNA generated and multiplexed RT-qPCR assay performed with taqman probes for SMN2 full length transcript, SMN2 Delta7 transcript, and reference GAPDH quantification.

Transcript levels (e.g., SMN2 full length transcript or SMN2 Delta7 transcript) were detected by RT-qPCR using Taqman. Specifically, RT-qPCR was performed for detecting GAPDH using Thermofisher TaqMan Gene Expression Assay Hs03929097_g1. RT-qPCR was performed for detecting SMN2 transcripts using custom Thermofisher TaqMan Gene Expression Assays with the following sequences:

SMN2-FL:
Forward Primer:
(SEQ ID NO: 10810)
5′ GCTCACATTCCTTAAATTAAGGAGAAA 3′
Reverse Primer:
(SEQ ID NO: 10811)
5′ TCCAGATCTGTCTGATCGTTTCTT 3′
Probe Sequence:
(SEQ ID NO: 10812)
5′ CTGGCATAGAGCAGCACTAAATGACACCAC 3′
SMN2 Delta7:
Forward Primer:
(SEQ ID NO: 10813)
5′ TGGCTATCATACTGGCTATTATATGGAA 3′
Reverse Primer:
(SEQ ID NO: 10811)
5′ TCCAGATCTGTCTGATCGTTTCTT 3′
Probe Sequence:
(SEQ ID NO: 10812)
5′ CTGGCATAGAGCAGCACTAAATGACACCAC 3′

RT-qPCR was performed on Applied Biosystems® 7500 Real-time PCR systems. One cycle of reverse transcription was performed at a temperature of 50° C. for 5 min. One cycle of RT inactivation/initial denaturation was performed at a temperature of 95° C. for 20 seconds. Forty five cycles of amplification were performed at a temperature of 95° C. for 1 second followed by 60° C. for 20 seconds.

SMN2-FL and SMN2 Delta7 (Ct) was normalized to GAPDH (deltaCt). To visualize the quantitative changes (e.g., % increase of SMN2-FL, decrease of SMN2 Delta7), the normalized SMN2-FL and SMN2 Delta7 signal was further normalized to the vehicle (treated with Endoporter alone, deltadeltaCt). Relative quantity of transcript level was calculated using the equation RQ=2^{-deltadeltaCt} and is used to describe the treatment condition comparison to normal, healthy levels (1.0).

TABLE 41
Performance of SMN2 LNA AONs evaluated in human derived iPSC motor neurons

| | | | | | | 200 nM ASO | | 50 nM ASO | |
|---|---|---|---|---|---|---|---|---|---|
| | | 200 nM ASO | | 50 nM SMN2 | | SMN2 | | SMN2 | |
| SEQ | | SMN2 FL RQ | | FL RQ | | Delta7 RQ | | Delta7 RQ | |
| ID NO: | Oligonucleotide Sequence* (5′ to 3′) | AVG | STD | AVG | STD | AVG | STD | AVG | STD |
| 10789 | GAT[LNA-T]CACTTTCATAATGCT[LNA-G]GCA | 1.114 | 0.887 | 1.498 | 0.685 | 0.042 | 0.033 | 0.107 | 0.050 |
| 10790 | GAT[LNA-T]CACTTTC[LNA-A]TAATGCT | 1.458 | 0.018 | 1.271 | 0.480 | 0.049 | 0.016 | 0.095 | 0.032 |
| | [LNA-G]GCA | | | | | | | | |
| 10791 | GATTCA[LNA-C]TTTCATA[LNA-A]TGCTGGCA | 1.796 | 0.823 | 1.984 | 0.245 | 0.035 | 0.016 | 0.066 | 0.030 |
| 10792 | GATTCA[LNA-C]TTTCATAAT[LNA-G]CTGGCA | 0.760 | 0.690 | 1.813 | 0.217 | 0.024 | 0.016 | 0.068 | 0.035 |
| 10793 | GATTCACT[LNA-T]TCATAAT[LNA-G]CTGGCA | 1.168 | 0.185 | 1.166 | 0.359 | 0.034 | 0.013 | 0.081 | 0.021 |
| 10794 | GAT[LNA-T]CACSTTCATAASGCT[LNA-G]GCA | 0.667 | 0.482 | 1.851 | 1.068 | 0.003 | 0.006 | 0.058 | 0.028 |
| 10795 | GAT[LNA-T]CACSTTC[LNA-A]TAASGCT | 1.397 | 0.422 | 1.770 | 0.652 | 0.019 | 0.007 | 0.044 | 0.029 |
| | [LNA-G]GCA | | | | | | | | |
| 10796 | GATTCA[LNA-C]STTCATA[LNA-A]SGCTGGCA | 1.415 | 1.091 | 2.279 | 0.522 | 0.034 | 0.040 | 0.058 | 0.055 |
| 10797 | GATTCA[LNA-C]STTCATAAS[LNA-G]CTGGCA | 0.952 | 0.082 | 1.623 | 1.070 | 0.000 | 0.000 | 0.056 | 0.036 |
| 10798 | GATTCACS[LNA-T]TCATAAS[LNA-G]CTGGCA | 1.609 | 0.579 | 1.869 | 0.479 | 0.041 | 0.028 | 0.029 | 0.016 |
| 10799 | AGA[LNA-T]TCACTTTCATAATGC[LNA-T]GGC | 1.283 | 0.974 | 1.812 | 1.410 | 0.015 | 0.026 | 0.047 | 0.041 |
| 10800 | AGA[LNA-T]TCACTTT[LNA-C]ATAATGC | 1.895 | 1.301 | 2.693 | 1.431 | 0.065 | 0.093 | 0.118 | 0.037 |
| | [LNA-T]GGC | | | | | | | | |
| 10801 | AGATTC[LNA-A]CTTTCAT[LNA-A]ATGCTGGC | 2.834 | 0.193 | 3.735 | 0.618 | 0.028 | 0.020 | 0.094 | 0.072 |
| 10802 | AGATTC[LNA-A]CTTTCATAA[LNA-T]GCTGGC | 2.554 | 0.870 | 2.924 | 0.509 | 0.043 | 0.025 | 0.139 | 0.061 |
| 10803 | AGATTCAC[LNA-T]TTCATAA[LNA-T]GCTGGC | 1.885 | 0.772 | 3.131 | 0.236 | 0.028 | 0.025 | 0.093 | 0.052 |
| 10804 | AGA[LNA-T]TCASTTTCATASTGC[LNA-T]GGC | 1.798 | 0.235 | 2.098 | 0.213 | 0.027 | 0.008 | 0.094 | 0.013 |
| 10805 | AGA[LNA-T]TCASTTT[LNA-C]ATASTGC | 2.242 | 0.068 | 1.519 | 0.814 | 0.030 | 0.016 | 0.036 | 0.009 |
| | [LNA-T]GGC | | | | | | | | |
| 10806 | AGATTC[LNA-A]STTTCAT[LNA-A]STGCTGGC | 1.802 | 0.212 | 1.678 | 0.163 | 0.011 | 0.002 | 0.053 | 0.019 |
| 10807 | AGATTC[LNA-A]STTTCATAS[LNA-T]GCTGGC | 1.437 | 0.298 | 1.190 | 0.425 | 0.000 | 0.000 | 0.039 | 0.023 |
| 10808 | AGATTCAS[LNA-T]TTCATAS[LNA-T]GCTGGC | 2.464 | 0.444 | 0.571 | 0.435 | 0.010 | 0.010 | 0.021 | 0.018 |

*Unless otherwise noted, each of the nucleosides of antisense oligonucleotides shown in Table 37 are modified nucleosides with 2′-O-(2-methoxyethyl) (2′-MOE) sugar moieties, each ″C″ is replaced with a 5-methylcytosine (5-MeC), and all internucleoside linkages are phosphorothioate linkages. A spacer, as indicated by S, is not a nucleoside. S represents a spacer of Formula (Iia′) as disclosed herein. [LNA-X] refers to a locked nucleic acid with a corresponding base X (e.g., LNA-A refers to a locked nucleic acid with an adenine nucleobase, LNA-G refers to a locked nucleic acid with a guanine nucleobase, LNA-T refers to a locked nucleic acid with a thymine nucleobase, and LNA-C refers to a locked nucleic acid with a 5-methylcytosine nucleobase).

What is claimed is:

1. A compound comprising a splice-switching oligonucleotide, wherein the splice-switching oligonucleotide comprises a spacer.

2. The compound of claim 1, wherein the splice-switching oligonucleotide comprises a sequence that is at least 85% complementary to an equal length portion of a sequence from a transcript whose mis-splicing leads to a neurological disease or a transcript whose splicing is capable of being modulated to treat a neurological disease.

3. The compound of claim 1, wherein the splice-switching oligonucleotide comprises a sequence that is at least 85% complementary to an equal length portion of a sequence from a transcript regulated by TDP-43, wherein at least one (i.e., one or more) nucleoside linkage of the oligonucleotide is a non-natural linkage, and further wherein the oligonucleotide comprises a spacer.

4. The compound of claim 1 or 2, wherein the modified oligonucleotide comprises a sequence that is at least 85% complementary to an equal length portion of a sequence from a transcript of any one STMN2, KCNQ2, UNC13A, or SMN2.

5. The compound of claim 1-2 or 4, wherein the modified oligonucleotide comprises a sequence that is at least 85% complementary to an equal length portion of any one of SEQ ID NO: 1339 or SEQ ID NO: 1341, SEQ ID NO: 3032-3043, SEQ ID NO: 9587-9595, or SEQ ID NO: 9698-9707.

6. A compound comprising a modified oligonucleotide comprising a sequence that is at least 85% complementary to an equal length portion of a sequence from a transcript of any one STMN2, KCNQ2, UNC13A, or SMN2, wherein at least one (i.e., one or more) nucleoside linkage of the oligonucleotide is a non-natural linkage, and further wherein the oligonucleotide comprises a spacer.

7. The compound of claim 6, wherein the modified oligonucleotide comprises a sequence that is at least 85% complementary to an equal length portion of any one of SEQ ID NO: 1339 or SEQ ID NO: 1341, SEQ ID NO: 3032-3043, SEQ ID NO: 9587-9595, or SEQ ID NO: 9698-9707, a sequence having 90% identity thereof, or to a 15 to 50 contiguous nucleobase portion thereof.

8. A splice-switching oligonucleotide, wherein the splice-switching oligonucleotide comprises a spacer.

9. The oligonucleotide of claim 8, wherein the oligonucleotide comprises a sequence that is at least 85% complementary to an equal length portion of a sequence from a transcript whose mis-splicing leads to a neurological disease or a transcript whose splicing is capable of being modulated to treat a neurological disease.

10. The oligonucleotide of claim 8, wherein the oligonucleotide comprises a sequence that is at least 85% complementary to an equal length portion of a sequence from a transcript regulated by TDP-43, wherein at least one (i.e., one or more) nucleoside linkage of the oligonucleotide is a non-natural linkage, and further wherein the oligonucleotide comprises a spacer.

11. The oligonucleotide of claim 8 or 9, wherein the oligonucleotide comprises a sequence that is at least 85% complementary to an equal length portion of a sequence from a transcript of any one STMN2, KCNQ2, UNC13A, or SMN2.

12. The oligonucleotide of claim 8-9 or 11, wherein the modified oligonucleotide comprises a sequence that is at least 85% complementary to an equal length portion of any one of SEQ ID NO: 1339 or SEQ ID NO: 1341, SEQ ID NO: 3032-3043, SEQ ID NO: 9587-9595, or SEQ ID NO: 9698-9707.

13. A splice-switching oligonucleotide comprising a sequence that is at least 85% complementary to an equal length portion of a sequence from a transcript of any one STMN2, KCNQ2, UNC13A, or SMN2, wherein at least one (i.e., one or more) nucleoside linkage of the oligonucleotide is a non-natural linkage, and further wherein the oligonucleotide comprises a spacer.

14. The oligonucleotide of claim 13, wherein the modified oligonucleotide comprises a sequence that is at least 85% complementary to an equal length portion of any one of SEQ ID NO: 1339 or SEQ ID NO: 1341, SEQ ID NO: 3032-3043, SEQ ID NO: 9587-9595, or SEQ ID NO: 9698-9707.

15. An oligonucleotide comprising a sequence that is at least 85% complementary to an equal length portion of any one of SEQ ID NO: 1339 or SEQ ID NO: 1341, SEQ ID NO: 3032-3043, SEQ ID NO: 9587-9595, or SEQ ID NO: 9698-9707, a sequence having 90% identity thereof, or to a 15 to 50 contiguous nucleobase portion thereof, wherein at least one (i.e., one or more) nucleoside linkage of the oligonucleotide is a non-natural linkage, and further wherein the oligonucleotide comprises a spacer.

16. A compound comprising a splice-switching oligonucleotide comprising a sequence that is at least 85% complementary to an equal length portion of a sequence from a transcript whose mis-splicing leads to a neurological disease or a transcript whose splicing is capable of being modulated to treat a neurological disease, wherein at least one (i.e., one or more) nucleoside linkage of the oligonucleotide is a non-natural linkage.

17. A compound comprising a splice-switching oligonucleotide comprising a sequence that is at least 85% complementary to an equal length portion of a sequence from a transcript regulated by TDP-43, wherein at least one (i.e., one or more) nucleoside linkage of the oligonucleotide is a non-natural linkage.

18. The compound of claim 16, wherein the splice-switching oligonucleotide comprises a sequence that is at least 85% complementary to an equal length portion of a sequence from a transcript of any one of SEQ ID NO: 1339 or SEQ ID NO: 1341, SEQ ID NO: 3032-3043, SEQ ID NO: 9587-9595, or SEQ ID NO: 9698-9707.

19. The compound of claim 16 or 18, wherein the modified splice-switching oligonucleotide comprises a sequence that is at least 85% complementary to an equal length portion of any one of SEQ ID NO: 1339 or SEQ ID NO: 1341, SEQ ID NO: 3032-3043, SEQ ID NO: 9587-9595, or SEQ ID NO: 9698-9707.

20. A compound comprising a modified splice-switching oligonucleotide comprising a sequence that is at least 85% complementary to an equal length portion of a sequence from a transcript of any one of STMN2, KCNQ2, UNC13A, or SMN2, wherein at least one (i.e., one or more) nucleoside linkage of the oligonucleotide is a non-natural linkage.

21. The compound of claim 20, wherein the modified oligonucleotide comprises a sequence that is at least 85% complementary to an equal length portion of any one of SEQ ID NO: 1339 or SEQ ID NO: 1341, SEQ ID NO: 3032-3043, SEQ ID NO: 9587-9595, or SEQ ID NO: 9698-9707, a sequence having 90% identity thereof, or to a 15 to 50 contiguous nucleobase portion thereof.

22. A splice-switching oligonucleotide comprising a sequence that is at least 85% complementary to an equal length portion of a sequence from a transcript whose mis-splicing leads to a neurological disease or a transcript whose splicing is capable of being modulated to treat a neurological disease, wherein at least one (i.e., one or more) nucleoside linkage of the oligonucleotide is a non-natural linkage.

23. A splice-switching oligonucleotide comprising a sequence that is at least 85% complementary to an equal length portion of a sequence from a transcript regulated by TDP-43, wherein at least one (i.e., one or more) nucleoside linkage of the oligonucleotide is a non-natural linkage.

24. The oligonucleotide of claim 23, wherein the oligonucleotide comprises a sequence that is at least 85% complementary to an equal length portion of a sequence from a transcript of any one STMN2, KCNQ2, UNC13A, or SMN2.

25. The oligonucleotide of claim 22 or 24, wherein the modified oligonucleotide comprises a sequence that is at least 85% complementary to an equal length portion of any one of SEQ ID NO: 1339 or SEQ ID NO: 1341, SEQ ID NO: 3032-3043, SEQ ID NO: 9587-9595, or SEQ ID NO: 9698-9707.

26. A splice-switching oligonucleotide comprising a sequence that is at least 85% complementary to an equal length portion of a sequence from a transcript of any one of STMN2, KCNQ2, UNC13A, or SMN2, wherein at least one (i.e., one or more) nucleoside linkage of the oligonucleotide is a non-natural linkage.

27. The oligonucleotide of claim 26, wherein the oligonucleotide comprises a sequence that is at least 85% complementary to an equal length portion of any one of SEQ ID NO: 1339 or SEQ ID NO: 1341, SEQ ID NO: 3032-3043, SEQ ID NO: 9587-9595, or SEQ ID NO: 9698-9707.

28. A splice-switching oligonucleotide comprising a sequence that is at least 85% complementary to an equal length portion of any one of SEQ ID NO: 1339 or SEQ ID NO: 1341, SEQ ID NO: 3032-3043, SEQ ID NO: 9587-9595, or SEQ ID NO: 9698-9707, wherein at least one (i.e., one or more) nucleoside linkage of the oligonucleotide is a non-natural linkage.

29. The oligonucleotide of claim 28, further comprising a spacer.

30. The compound or oligonucleotide of claims 1-29, wherein the oligonucleotide comprises a segment with at most 11 linked nucleosides.

31. The compound or oligonucleotide of claims 1-29, wherein the oligonucleotide comprises a segment with at most 10, 9, or 8 linked nucleosides.

32. The compound or oligonucleotide of claims 1-29, wherein the oligonucleotide comprises a segment with at most 7 linked nucleosides.

33. The compound or oligonucleotide of claims 1-29, wherein the oligonucleotide comprises a segment with at most 6, 5, 4, 3, or 2 linked nucleosides.

34. The compound or oligonucleotide of claims 1-33, wherein every segment of the oligonucleotide comprises at most 7 linked nucleosides.

35. The compound or oligonucleotide of any one of claims 30-34, wherein the oligonucleotide comprises a sequence that shares at least 85% identity with an equal length portion of any one of SEQ ID NOs: 1-466, SEQ ID NOs: 893-1338, SEQ ID NOs: 1342-1366, SEQ ID NOs: 1392-1664, or SEQ ID NOs: 10655-10669, SEQ ID NOs: 1676-1851, SEQ ID NOs: 2028-2529, SEQ ID NOs: 3046-3221, SEQ ID NOs: 3398-3899, and SEQ ID NOs: 4402-4530, SEQ ID NOs: 4531-5794, SEQ ID NOs: 7059-8322, SEQ ID NOs: 9596-9603, SEQ ID NOs: 9710-10141, SEQ ID NOs: 10574-10651, SEQ ID NOs: 10670-10779, SEQ ID NOs: 10783-10808, and SEQ ID NOs: 10814-10820.

36. The compound or oligonucleotide of any one of claims 30-35, wherein the oligonucleotide comprises a sequence that shares at least 90% identity with an equal length portion of any one of SEQ ID NOs: 1-466, SEQ ID NOs: 893-1338, SEQ ID NOs: 1342-1366, SEQ ID NOs: 1392-1664, or SEQ ID NOs: 10655-10669, SEQ ID NOs: 1676-1851, SEQ ID NOs: 2028-2529, SEQ ID NOs: 3046-3221, SEQ ID NOs: 3398-3899, and SEQ ID NOs: 4402-4530, SEQ ID NOs: 4531-5794, SEQ ID NOs: 7059-8322, SEQ ID NOs: 9596-9603, SEQ ID NOs: 9710-10141, SEQ ID NOs: 10574-10651, SEQ ID NOs: 10670-10779, SEQ ID NOs: 10783-10808, and SEQ ID NOs: 10814-10820.

37. The compound or oligonucleotide of any one of claims 30-36, wherein the oligonucleotide comprises a sequence that shares at least 95% identity with an equal length portion of any one of SEQ ID NOs: 1-466, SEQ ID NOs: 893-1338, SEQ ID NOs: 1342-1366, SEQ ID NOs: 1392-1664, or SEQ ID NOs: 10655-10669, SEQ ID NOs: 1676-1851, SEQ ID NOs: 2028-2529, SEQ ID NOs: 3046-3221, SEQ ID NOs: 3398-3899, and SEQ ID NOs: 4402-4530, SEQ ID NOs: 4531-5794, SEQ ID NOs: 7059-8322, SEQ ID NOs; 9596-9603, SEQ ID NOs: 9710-10141, SEQ ID NOs: 10574-10651, SEQ ID NOs: 10670-10779, SEQ ID NOs: 10783-10808, and SEQ ID NOs: 10814-10820.

38. The compound or oligonucleotide of any one of claims 30-36, wherein the oligonucleotide comprises a sequence that shares 100% identity with an equal length portion of any one of SEQ ID NOs: 1-466, SEQ ID NOs: 893-1338, SEQ ID NOs: 1342-1366, SEQ ID NOs: 1392-1664, or SEQ ID NOs: 10655-10669, SEQ ID NOs: 1676-1851, SEQ ID NOs: 2028-2529, SEQ ID NOs: 3046-3221, SEQ ID NOs: 3398-3899, and SEQ ID NOs: 4402-4530, SEQ ID NOs: 4531-5794, SEQ ID NOs: 7059-8322, SEQ ID NOs: 9596-9603, SEQ ID NOs: 9710-10141, SEQ ID NOs: 10574-10651, SEQ ID NOs: 10670-10779, SEQ ID NOs: 10783-10808, and SEQ ID NOs: 10814-10820.

39. The compound or oligonucleotide of any one of claims 1-38, wherein the oligonucleotide comprises a segment with at most 11 linked nucleosides or at most 7 linked nucleosides, and wherein the oligonucleotide comprises a sequence that shares at least 85% identity with an equal length portion of any one of SEQ ID NOs: 1-466, SEQ ID NOs: 893-1338, SEQ ID NOs: 1342-1366, SEQ ID NOs: 1392-1664, or SEQ ID NOs: 10655-10669, SEQ ID NOs: 1676-1851, SEQ ID NOs: 2028-2529, SEQ ID NOs: 3046-3221, SEQ ID NOs: 3398-3899, and SEQ ID NOs: 4402-4530, SEQ ID NOs: 4531-5794, SEQ ID NOs: 7059-8322, SEQ ID NOs: 9596-9603, SEQ ID NOs: 9710-10141, SEQ ID NOs: 10574-10651, SEQ ID NOs: 10670-10779, SEQ ID NOs: 10783-10808, and SEQ ID NOs: 10814-10820.

40. The compound or oligonucleotide of claim 39, wherein the oligonucleotide comprises a segment with at most 6, 5, 4, 3, or 2 linked nucleosides, and wherein the oligonucleotide comprises a sequence that shares at least 85% identity with an equal length portion of any one of SEQ ID NOs: 1-466, SEQ ID NOs: 893-1338, SEQ ID NOs: 1342-1366, SEQ ID NOs: 1392-1664, or SEQ ID NOs: 10655-10669, SEQ ID NOs: 1676-1851, SEQ ID NOs: 2028-2529, SEQ ID NOs: 3046-3221, SEQ ID NOs: 3398-3899, and SEQ ID NOs: 4402-4530, SEQ ID NOs: 4531-5794, SEQ ID NOs: 7059-8322, SEQ ID NOs: 9596-9603, SEQ ID NOs: 9710-10141, SEQ ID NOs: 10574-10651, SEQ ID NOs: 10670-10779, SEQ ID NOs: 10783-10808, and SEQ ID NOs: 10814-10820.

41. The compound or oligonucleotide of claim 39 or 40, wherein the oligonucleotide comprises a segment with at most 6, 5, 4, 3, or 2 linked nucleosides, and wherein the oligonucleotide comprises a sequence that shares at least 90% identity with an equal length portion of any one of SEQ ID NOs: 1-466, SEQ ID NOs: 893-1338, SEQ ID NOs: 1342-1366, SEQ ID NOs: 1392-1664, or SEQ ID NOs: 10655-10669, SEQ ID NOs: 1676-1851, SEQ ID NOs: 2028-2529, SEQ ID NOs: 3046-3221, SEQ ID NOs: 3398-3899, and SEQ ID NOs: 4402-4530, SEQ ID NOs: 4531-5794, SEQ ID NOs: 7059-8322, SEQ ID NOs: 9596-9603, SEQ ID NOs: 9710-10141, SEQ ID NOs: 10574-10651, SEQ ID NOs: 10670-10779, SEQ ID NOs: 10783-10808, and SEQ ID NOs: 10814-10820.

42. The compound or oligonucleotide of any one of claims 1-41, wherein the oligonucleotide is at least 18, at least 19, at least 20, at least 21, at least 22, at least 23, at least 24, or at least 25 oligonucleotide units in length.

43. The compound or oligonucleotide of claim 42, wherein the oligonucleotide is at least 19 oligonucleotide units in length.

44. The compound or oligonucleotide of any one of claims 1-43, wherein the spacer is a nucleoside-replacement group comprising a non-sugar substitute that is incapable of linking to a nucleotide base.

45. The compound or oligonucleotide of claim 44, wherein the spacer is located between positions 10 and 15 of the oligonucleotide.

46. The compound or oligonucleotide of claim 44, wherein the spacer is located between positions 7 and 11 of the oligonucleotide.

47. The compound or oligonucleotide of claim 44-46, wherein the oligonucleotide further comprises a second spacer, wherein the second spacer is located between positions 14 and 22 of the oligonucleotide.

48. The compound or oligonucleotide of claim 47, wherein the spacer and the second spacer are separated by at least 5 nucleobases, at least 6 nucleobases, or at least 7 nucleobases in the oligonucleotide.

49. The compound or oligonucleotide of claim 47 or 48, wherein the spacer is located between positions 7 and 9 of the oligonucleotide, and wherein the second spacer is located between positions 15 and 18 of the oligonucleotide.

50. The compound or oligonucleotide of any one of claims 47-49, wherein the spacer is located at position 8 of the oligonucleotide, and wherein the second spacer is located at position 16 of the oligonucleotide.

51. The compound or oligonucleotide of any one of claims 47-50, wherein the oligonucleotide further comprises a third spacer, wherein the third spacer is located between positions 21 and 24 of the oligonucleotide.

52. The compound or oligonucleotide of claim 44, wherein the spacer is located between positions 2 and 5 of the oligonucleotide.

53. The compound or oligonucleotide of claim 52, wherein the oligonucleotide further comprises a second spacer, wherein the second spacer is located between positions 8 and 12 of the oligonucleotide.

54. The compound or oligonucleotide of claim 53, wherein the oligonucleotide further comprises a third spacer, wherein the third spacer is located between positions 18 and 22 of the oligonucleotide.

55. The compound or oligonucleotide of claim 44, wherein the oligonucleotide further comprises a second spacer and a third spacer, wherein the three spacers are located at positions in the oligonucleotide such that each segment of the oligonucleotide has at most 7 linked nucleosides.

56. The compound or oligonucleotide of claim 55, wherein at least two of the three spacers are adjacent to a guanine nucleobase.

57. The compound or oligonucleotide of claim 56, wherein each of the at least two of the three spacers immediately precede a guanine nucleobase.

58. The compound or oligonucleotide of any one of claims 44-57, wherein each of the first, second or third spacers is a nucleoside-replacement group comprising a non-sugar substitute wherein the non-sugar substitute does not contain a ketone, aldehyde, ketal, hemiketal, acetal, hemiacetal, aminal or hemiaminal moiety and is incapable of forming a covalent bond with a nucleotide base.

59. The compound or oligonucleotide of any one of claims 44-57, wherein each of the first, second or third spacers is independently represented by Formula (X), wherein: 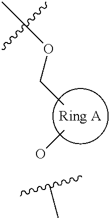
Ring A is an optionally substituted 4-8 member monocyclic cycloalkyl group or a 4-8 member monocyclic heterocyclyl group, wherein the heterocyclyl group contains 1 or 2 heteroatoms selected from O, S and N, provided that A is not capable of forming a covalent bond to a nucleobase; and the 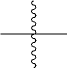
symbol represents the point of connection to an internucleoside linkage.

60. The compound or oligonucleotide of claim 59, wherein each of the first, second or third spacers is independently represented by Formula (Xa), wherein:

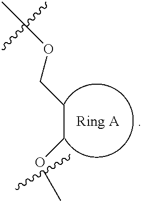

61. The compound or nucleotide of claim 59 or 60, wherein ring A is an optionally substituted 4-8 member monocyclic cycloalkyl group selected from cyclobutyl, cyclopentyl, cyclohexyl, cycloheptyl and cyclooctyl; or a 4-8 member monocyclic heterocyclyl group, selected from oxetanyl, tetrahydrofuranyl, tetrahydropyranyl, 1,4-dioxanyl, pyrrolidinyl, piperidinyl, piperazinyl, morpholinyl and azepanyl.

62. The compound or nucleotide of claim 61 wherein ring A is tetrahydrofuranyl.

63. The compound or nucleotide of claim 61 wherein ring A is tetrahydropyranyl.

64. The compound or oligonucleotide of any one of claims 44-57, wherein each of the first, second or third spacers is independently represented by Formula I, wherein: 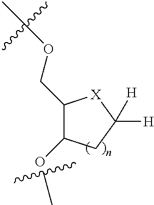
X is selected from —CH₂— and —O—; and
n is 0, 1, 2 or 3.

65. The compound or oligonucleotide of any one of claims 44-57, wherein each of the first, second or third spacers is independently represented by Formula I′, wherein: 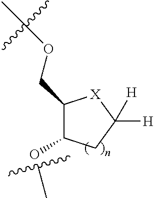
X is selected from —CH₂— and —O—; and
n is 0, 1, 2 or 3.

66. The compound or oligonucleotide of any one of claims 44-57, wherein each of the first, second or third spacers is independently represented by Formula (Ia), wherein: 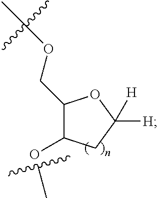
and
n is 0, 1, 2 or 3.

67. The compound or oligonucleotide of any one of claims 44-57, wherein each of the first, second or third spacers is independently represented by Formula (Ia′), wherein: 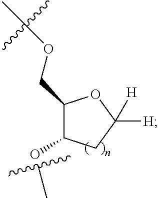
and
n is 0, 1, 2 or 3.

68. The compound or oligonucleotide of any one of claims 44-57, wherein each of the first, second or third spacers is independently represented by Formula II, wherein: 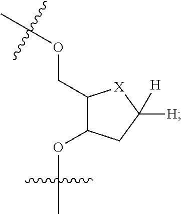
and
X is selected from —CH₂— and —O—.

69. The compound or oligonucleotide of any one of claims 44-57, wherein each of the first, second or third spacers is independently represented by Formula II′, wherein: 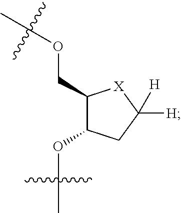
and
X is selected from —CH₂— and —O—.

70. The compound or oligonucleotide of any one of claims 44-57, wherein each of the first, second or third spacers is independently represented by Formula (Iia), wherein:

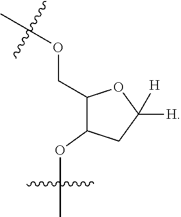

71. The compound or oligonucleotide of any one of claims 44-57, wherein each of the first, second or third spacers is independently represented by Formula (Iia′), wherein:

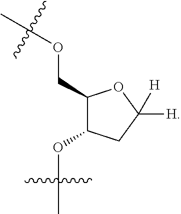

72. The compound or oligonucleotide of any one of claims 44-57, wherein each of the first, second or third spacers is independently represented by Formula (IIi), wherein: 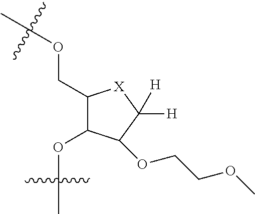
X is selected from —CH₂— and —O—.

73. The compound or oligonucleotide of any one of claims 44-57, wherein each of the first, second or third spacers is independently represented by Formula (IIi′), wherein: 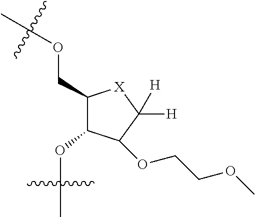
X is selected from —CH₂— and —O—.

74. The compound or oligonucleotide of any one of claims 44-57, wherein each of the first, second or third spacers is independently represented by Formula (IIib), wherein:

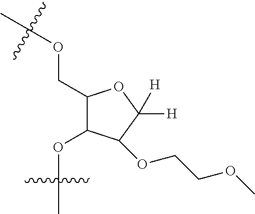 [SMILES: *OCC1OCC(OCCOC)C1O*]

75. The compound or oligonucleotide of any one of claims 44-57, wherein each of the first, second or third spacers is independently represented by Formula (IIib′), wherein:

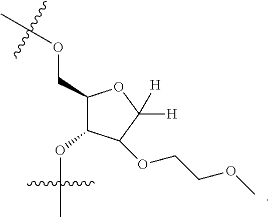 [SMILES: *OC[C@H]1OCC(OCCOC)[C@@H]1O*]

76. The compound or oligonucleotide of any one of claims 44-57, wherein each of the first, second or third spacers is independently represented by Formula III, wherein: 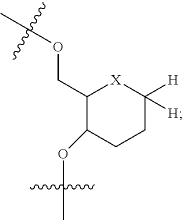
and
X is selected from —CH₂— and —O—.

77. The compound or oligonucleotide of any one of claims 44-57, wherein each of the first, second or third spacers is independently represented by Formula III′, wherein: 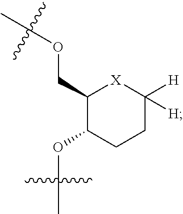
and
X is selected from —CH₂— and —O—.

78. The compound or oligonucleotide of any one of claims 44-57, wherein each of the first, second or third spacers is independently represented by Formula (IIIa), wherein:

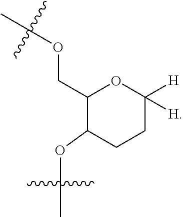

79. The compound or oligonucleotide of any one of claims 44-57, wherein each of the first, second or third spacers is independently represented by Formula (IIIa′), wherein:

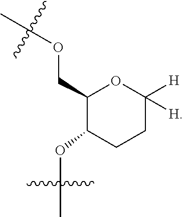

80. The compound or oligonucleotide of any one of the above claims, wherein the oligonucleotide further comprises a locked nucleic acid (LNA).

81. The compound or oligonucleotide of claim 80, wherein the locked nucleic acid (LNA) is located at one of positions 4, 7, 9, 12, 15, or 20 of the oligonucleotide.

82. The compound or oligonucleotide of any one of the above claims, wherein the oligonucleotide comprising the spacer has a GC content of at least 10%.

83. The compound or oligonucleotide of any one of the above claims, wherein the oligonucleotide comprising the spacer has a GC content of at least 20%.

84. The compound or oligonucleotide of any one of the above claims, wherein the oligonucleotide comprising the spacer has a GC content of at least 25%.

85. The compound or oligonucleotide of any one of the above claims, wherein the oligonucleotide comprising the spacer has a GC content of at least 30%.

86. The compound or oligonucleotide of any one of the above claims, wherein the oligonucleotide comprising the spacer has a GC content of at least 40%.

87. The compound or oligonucleotide of any one of the above claims, wherein the oligonucleotide comprising the spacer has a GC content of at least 50%.

88. The compound or oligonucleotide of any one of the above claims, wherein the oligonucleotide is between 12 and 40 oligonucleotide units in length.

89. The compound or oligonucleotide of any one of the above claims, wherein at least one (i.e., one or more) nucleoside linkage of the oligonucleotide is independently selected from the group consisting of a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

90. The compound or oligonucleotide of any one of claims 1-89, wherein one or more nucleoside linkages that link a base at position 3 or position 4 of the oligonucleotide are phosphodiester linkages.

91. The compound or oligonucleotide of claim 90, wherein only one nucleoside linkage that links a base at position 3 or position 4 of the oligonucleotide is a phosphodiester linkage.

92. The compound or oligonucleotide of any one of claims 1-89, wherein nucleoside linkages that link bases at both position 3 and position 4 of the oligonucleotide are phosphodiester linkages.

93. The compound or oligonucleotide of any one of claims 1-89, wherein one or more bases immediately preceding a spacer in the oligonucleotide are linked through phosphodiester bonds.

94. The compound or oligonucleotide of claim 93, wherein only the base immediately preceding the spacer in the oligonucleotide is linked to the spacer through a phosphodiester bond.

95. The compound or oligonucleotide of claim 94, wherein the base immediately preceding the spacer in the oligonucleotide is further linked to a further preceding base through a phosphodiester bond.

96. The compound or oligonucleotide of claim 94, wherein the oligonucleotide comprises a second spacer, wherein a base immediately preceding the second spacer is linked to a further preceding base through a phosphodiester bond.

97. The compound or oligonucleotide of any one of claims 1-89, wherein one or more bases immediately succeeding a spacer in the oligonucleotide are linked through phosphodiester bonds.

98. The compound or oligonucleotide of claim 97, wherein only the base immediately succeeding the spacer in the oligonucleotide is linked to the spacer through a phosphodiester bond.

99. The compound or oligonucleotide of claim 93, wherein two bases immediately preceding the spacer in the oligonucleotide are linked through phosphodiester bonds.

100. The compound or oligonucleotide of any one of claims 1-89, wherein one or more bases immediately preceding a spacer in the oligonucleotide are linked through phosphodiester bonds and wherein one or more bases immediately succeeding the spacer in the oligonucleotide are linked through phosphodiester bonds.

101. The compound or oligonucleotide of claim 100, wherein one base immediately preceding the spacer and one base immediately succeeding the spacer are linked through phosphodiester bonds.

102. The compound or oligonucleotide of claim 100 or 101, wherein the oligonucleotide includes a second spacer, and wherein one or more bases immediately preceding the second spacer in the oligonucleotide are linked through phosphodiester bonds and wherein one or more bases immediately succeeding the second spacer in the oligonucleotide are linked through phosphodiester bonds.

103. The compound or oligonucleotide of claim 102, wherein one base immediately preceding the second spacer and one base immediately succeeding the second spacer are linked through phosphodiester bonds.

104. The compound or oligonucleotide of any one of claims 1-89, wherein the oligonucleotide comprises a range of bases that are linked through phosphodiester bonds, the range of bases comprising at least two bases.

105. The compound or oligonucleotide of any one of claims 1-89, wherein the oligonucleotide comprises a range of bases that are linked through phosphodiester bonds, the range of bases comprising at least five bases.

106. The compound or oligonucleotide of claim 104 or 105, wherein the oligonucleotide comprises two or more spacers, and wherein the range of bases are positioned between the at least two spacers.

107. The compound or oligonucleotide of any one of the above claims, wherein one or more internucleoside linkage of the oligonucleotide is a modified internucleoside linkage.

108. The compound or oligonucleotide of claim 107, wherein the modified internucleoside linkage of the oligonucleotide is a phosphorothioate linkage.

109. The compound or oligonucleotide of claim 107 or 108, wherein all internucleoside linkages of the oligonucleotide are phosphorothioate linkages.

110. The compound or oligonucleotide of claim 108, wherein the phosphorothioate linkage is in one of a Rp configuration or a Sp configuration.

111. The compound or oligonucleotide of any one of the preceding claims, wherein the oligonucleotide comprises at least one modified sugar moiety.

112. The compound or oligonucleotide of claim 111, wherein the modified sugar moiety is one of a 2′-OMe modified sugar moiety, bicyclic sugar moiety, 2′-O-(2-methoxyethyl) (2′-MOE), 2′-deoxy-2′-fluoro nucleoside, 2′-fluoro-β-D-arabinonucleoside, locked nucleic acid (LNA), constrained ethyl 2′-4′-bridged nucleic acid (cEt), S-cEt, tcDNA, hexitol nucleic acids (HNA), and tricyclic analog (e.g., tcDNA).

113. The compound or oligonucleotide of any one of the above claims, wherein the oligonucleotide exhibits at least a 30%, 40%, 50%, 60%, 70%, 80%, or 90% increase of full length STMN2, KCNQ2, UNC13A, or SMN2 protein.

114. The compound or oligonucleotide of any one of the above claims, wherein the oligonucleotide exhibits at least a 100% increase of full length protein.

115. The compound or oligonucleotide of any one of the above claims, wherein the oligonucleotide exhibits at least a 200% increase of full length protein.

116. The compound or oligonucleotide of any one of the above claims, wherein the oligonucleotide exhibits at least a 300% increase of full length protein.

117. The compound or oligonucleotide of any one of the above claims, wherein the oligonucleotide exhibits at least a 400% increase of full length protein.

118. The compound or oligonucleotide of any one of claims 113-117, wherein increase of the full length protein is measured in comparison to a reduced level of full length protein achieved using a TDP43 antisense oligonucleotide.

119. The compound or oligonucleotide of any one of the above claims, wherein the oligonucleotide exhibits at least a 10%, 20%, 30%, 40%, 50%, 60%, 70%, 80%, 90%, or 100% rescue of full length protein.

120. The compound or oligonucleotide of any one of the above claims, wherein the oligonucleotide exhibits at least a 50%, 60%, 70%, 80%, or 90% reduction of a mis-spliced transcript.

121. A method of treating a neurological disease and/or a neuropathy in a patient in need thereof, the method comprising administering to the patient a compound or an oligonucleotide of any one of claims 1-120.

122. The method of claim 121, wherein the neurological disease is selected from the group consisting of amyotrophic lateral sclerosis (ALS), frontotemporal dementia (FTD), ALS with FTD, Alzheimer's disease (AD), Parkinson's disease (PD), Parkinson's Disease with dementia, dementia with lewy bodies, synucleinopathies, Huntington's disease, Brachial plexus injuries, peripheral nerve injuries, progressive supranuclear palsy (PSP), brain trauma, spinal cord injury, tuberous sclerosis complex, Pick's Disease, tauopathies, primary age-related tauopathy, Down Syndrome, epilepsy/seizure disorder, depression, traumatic brain injury (TBI), chronic traumatic encephalopathy (CTE), HIV-associated neurocognitive disorders (HAND), multisystem atrophy, amnestic mild cognitive impairment, corticobasal degeneration (CBD) and/or neuropathies such a chemotherapy induced neuropathy, Spinocerebellar ataxia (SCA), SCA type 2, Spinal Muscular Atrophy (SMA), Parkinsonism, Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), Limbic-predominant age-related TDP-43 encephalopathy (LATE)), Cerebral Age-Related TDP-43 With Sclerosis (CARTS), Gaucher's disease, and facial onset sensory and motor neuronopathy, Guam Parkinson-dementia complex, multisystem proteinopathy, Perry disease, and synaptic diseases like autism.

123. The method of claim 122, wherein the neurological disease is ALS.

124. The method of claim 122, wherein the neurological disease is FTD.

125. The method of claim 122, wherein the neurological disease is ALS with FTD.

126. The method of claim 122, wherein the neurological disease is AD.

127. The method of claim 122, wherein the neurological disease is PD.

128. The method of claim 122, wherein the neurological disease is spinal muscular atrophy (SMA).

129. The method of claim 121, wherein the neuropathy is chemotherapy induced neuropathy.

130. A method of restoring axonal outgrowth and/or regeneration of a neuron, the method comprising exposing the neuron to a compound or an oligonucleotide of any one of claims 1-120.

131. A method of increasing, promoting, stabilizing, or maintaining any one of STMN2, KCNQ2, UNC13A, or SMN2 expression and/or function in a neuron, the method comprising exposing the cell to a compound or an oligonucleotide of any one of claims 1-120.

132. The method of claim 130 or 131, wherein the neuron is a motor neuron.

133. The method of claim 130 or 131, wherein the neuron is a spinal cord neuron.

134. The method of any one of claims 130-133, wherein the neuron is a neuron of a patient in need of treatment of a neurological disease and/or a neuropathy.

135. The method of claim 134, wherein the neuropathy is chemotherapy induced neuropathy.

136. The method of any one of claims 130-135, wherein the exposing is performed in vivo or ex vivo.

137. The method of any one of claims 130-135, wherein the exposing comprises administering the oligonucleotide to a patient in need thereof.

138. The method of any one of claims 130-137, wherein the oligonucleotide is administered topically, parenterally, intrathecally, intrathalamically, intracisternally, orally, rectally, buccally, sublingually, vaginally, pulmonarily, intratracheally, intranasally, transdermally, intraduodenally, or intracerebroventricularly.

139. The method of claim 138, wherein the oligonucleotide is administered orally.

140. The method of any one of claims 130-138, wherein a therapeutically effective amount of the oligonucleotide is administered intrathecally, intrathalamically, intracerebroventricularly, or intracisternally.

141. The method of any one of claims 130-140, wherein the patient is a human.

142. A pharmaceutical composition comprising the oligonucleotide of any one of claims 1-120, or a pharmaceutically acceptable salt thereof, and a pharmaceutically acceptable excipient.

143. The pharmaceutical composition of claim 142, wherein the pharmaceutical composition is suitable for topical, intrathecal, intrathalamic, intracisternal, intracerebroventricular, parenteral, oral, pulmonary, intratracheal, intranasal, transdermal, rectal, buccal, sublingual, vaginal, or intraduodenal administration.

144. A method of treating a neurological disease or a neuropathy in a patient in need thereof, the method comprising administering to a patient in need thereof a therapeutically effective amount of a pharmaceutical composition of claim 142 or 143.

145. The method of claim 144, wherein the neurological disease is selected from the group consisting of amyotrophic lateral sclerosis (ALS), frontotemporal dementia (FTD), ALS with FTD, Alzheimer's disease (AD), Parkinson's disease (PD), Parkinson's Disease with dementia, dementia with lewy bodies, synucleinopathies, Huntington's disease, Brachial plexus injuries, peripheral nerve injuries, progressive supranuclear palsy (PSP), brain trauma, spinal cord injury, tuberous sclerosis complex, Pick's Disease, tauopathies, primary age-related tauopathy, Down Syndrome, epilepsy/seizure disorder, depression, traumatic brain injury (TBI), chronic traumatic encephalopathy (CTE), HIV-associated neurocognitive disorders (HAND), multisystem atrophy, amnestic mild cognitive impairment, corticobasal degeneration (CBD) and/or neuropathies such a chemotherapy induced neuropathy, Spinocerebellar ataxia (SCA), SCA type 2, Spinal Muscular Atrophy (SMA), Parkinsonism, Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), Limbic-predominant age-related TDP-43 encephalopathy (LATE)), Cerebral Age-Related TDP-43 With Sclerosis (CARTS), Gaucher's disease, and facial onset sensory and motor neuronopathy, Guam Parkinson-dementia complex, multisystem proteinopathy, Perry disease, and synaptic diseases like autism.

146. The method of claim 145, wherein the neurological disease is ALS.

147. The method of claim 145, wherein the neurological disease is FTD.

148. The method of claim 145, wherein the neurological disease is ALS with FTD.

149. The method of claim 145, wherein the neurological disease is AD.

150. The method of claim 145, wherein the neurological disease is PD.

151. The method of claim 145, wherein the neurological disease is spinal muscular atrophy (SMA).

152. The method of claim 144, wherein the neuropathy is chemotherapy induced neuropathy.

153. The method of any one of claims 144-152, wherein the pharmaceutical composition is administered topically, parenterally, orally, pulmonarily, rectally, buccally, sublingually, vaginally, intratracheally, intranasally, intracisternally, intrathecally, intrathalamically, transdermally, intraduodenally, or intracerebroventricularly.

154. The method of any one of claims 144-152, wherein the pharmaceutical composition is administered intrathecally, intrathalamically, or intracisternally.

155. The method of any one of claims 144-154, wherein a therapeutically effective amount of the oligonucleotide is administered intrathecally, intrathalamically or intracisternally.

156. The method of any one of claims 144-155, wherein the patient is human.

157. A method for treating a neurological disease in a subject in need thereof, the method comprising administering to the subject an oligonucleotide comprising a segment with at most 7 linked nucleosides, and wherein the oligonucleotide is at least 85% complementary to an equal length portion of a sequence from a transcript whose mis-splicing leads to a neurological disease or a transcript whose splicing is capable of being modulated to treat a neurological disease, or a pharmaceutically acceptable salt thereof;
wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or
wherein at least one (i.e., one or more) nucleoside is substituted with a component selected from the group consisting of a 2′-O-(2-methoxyethyl) nucleoside, a 2′-O-methyl nucleoside, a 2′-O—(N-methylacetamide) nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA)
optionally, wherein the oligonucleotide further comprises a spacer.

158. A method for treating a neurological disease in a subject in need thereof, the method comprising administering to the subject an oligonucleotide comprising a segment with at most 7 linked nucleosides, and wherein oligonucleotide is at least 85% complementary to a sequence from a transcript of any one STMN2, KCNQ2, UNC13A, or SMN2, or a pharmaceutically acceptable salt thereof;
wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or
wherein at least one (i.e., one or more) nucleoside is substituted with a component selected from the group consisting of a 2′-O-(2-methoxyethyl) nucleoside, a 2′-O-methyl nucleoside, a 2′-O—(N-methylacetamide) nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA)
optionally, wherein the oligonucleotide further comprises a spacer.

159. A method for treating a neurological disease in a subject in need thereof, the method comprising administering to the subject an oligonucleotide comprising a segment with at most 7 linked nucleosides, and wherein oligonucleotide shares at least 85% identity with any one of SEQ ID NOs: 1-466, SEQ ID NOs: 893-1338, SEQ ID NOs: 1342-1366, SEQ ID NOs: 1392-1664, or SEQ ID NOs: 10655-10669, SEQ ID NOs: 1676-1851, SEQ ID NOs: 2028-2529, SEQ ID NOs: 3046-3221, SEQ ID NOs: 3398-3899, and SEQ ID NOs: 4402-4530, SEQ ID NOs: 4531-5794, SEQ ID NOs: 7059-8322, SEQ ID NOs: 9596-9603, SEQ ID NOs: 9710-10141, SEQ ID NOs: 10574-10651, SEQ ID NOs: 10670-10779, SEQ ID NOs: 10783-10808, and SEQ ID NOs: 10814-10820, or a pharmaceutically acceptable salt thereof;
wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or
wherein at least one (i.e., one or more) nucleoside is substituted with a component selected from the group consisting of a 2′-O-(2-methoxyethyl) nucleoside, a 2′-O-methyl nucleoside, a 2′-O—(N-methylacetamide) nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA)
optionally, wherein the oligonucleotide further comprises a spacer.

160. A method for treating amyotrophic lateral sclerosis (ALS) in a subject in need thereof, the method comprising administering to the subject an oligonucleotide comprising a segment with at most 7 linked nucleosides, and wherein oligonucleotide shares at least 85% identity with any one of SEQ ID NOs: 1-466, SEQ ID NOs: 893-1338, SEQ ID NOs: 1342-1366, SEQ ID NOs: 1392-1664, or SEQ ID NOs: 10655-10669, SEQ ID NOs: 1676-1851, SEQ ID NOs: 2028-2529, SEQ ID NOs: 3046-3221, SEQ ID NOs: 3398-3899, and SEQ ID NOs: 4402-4530, or SEQ ID NOs: 4531-5794, SEQ ID NOs: 7059-8322, or SEQ ID NOs: 9596-9603, or a pharmaceutically acceptable salt thereof;
wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or
wherein at least one (i.e., one or more) nucleoside is substituted with a component selected from the group consisting of a 2′-O-(2-methoxyethyl) nucleoside, a 2′-O-methyl nucleoside, a 2′-O—(N-methylacetamide) nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA)
optionally, wherein the oligonucleotide further comprises a spacer.

161. A method for treating Alzheimer's Disease (AD) with frontotemporal dementia (FTD) in a subject in need thereof, the method comprising administering to the subject an oligonucleotide comprising a segment with at most 7 linked nucleosides, and wherein oligonucleotide shares at least 85% identity with any one of SEQ ID NOs: 1-466, SEQ ID NOs: 893-1338, SEQ ID NOs: 1342-1366, SEQ ID NOs: 1392-1664, or SEQ ID NOs: 10655-10669, SEQ ID NOs: 1676-1851, SEQ ID NOs: 2028-2529, SEQ ID NOs: 3046-3221, SEQ ID NOs: 3398-3899, and SEQ ID NOs: 4402-4530, or SEQ ID NOs: 4531-5794, SEQ ID NOs: 7059-8322, or SEQ ID NOs: 9596-9603, or a pharmaceutically acceptable salt thereof;
wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or
wherein at least one (i.e., one or more) nucleoside is substituted with a component selected from the group consisting of a 2′-O-(2-methoxyethyl) nucleoside, a 2′-O-methyl nucleoside, a 2′-O—(N-methylacetamide) nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA), optionally, wherein the oligonucleotide further comprises a spacer

162. A method for treating frontotemporal dementia (FTD) in a subject in need thereof, the method comprising administering to the subject an oligonucleotide comprising a segment with at most 7 linked nucleosides, and wherein oligonucleotide shares at least 85% identity with any one of SEQ ID NOs: 1-466, SEQ ID NOs: 893-1338, SEQ ID NOs: 1342-1366, SEQ ID NOs: 1392-1664, or SEQ ID NOs: 10655-10669, SEQ ID NOs: 1676-1851, SEQ ID NOs: 2028-2529, SEQ ID NOs: 3046-3221, SEQ ID NOs: 3398-3899, and SEQ ID NOs: 4402-4530, or SEQ ID NOs: 4531-5794, SEQ ID NOs: 7059-8322, or SEQ ID NOs: 9596-9603, or a pharmaceutically acceptable salt thereof;
wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or
wherein at least one (i.e., one or more) nucleoside is substituted with a component selected from the group consisting of a 2′-O-(2-methoxyethyl) nucleoside, a 2′-O-methyl nucleoside, a 2′-O—(N-methylacetamide) nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA)
optionally, wherein the oligonucleotide further comprises a spacer.

163. A method for treating amyotrophic lateral sclerosis (ALS) with frontotemporal dementia (FTD) in a subject in need thereof, the method comprising administering to the subject an oligonucleotide comprising a segment with at most 7 linked nucleosides, and wherein oligonucleotide shares at least 85% identity with any one of SEQ ID NOs: 1-466, SEQ ID NOs: 893-1338, SEQ ID NOs: 1342-1366, SEQ ID NOs: 1392-1664, or SEQ ID NOs: 10655-10669, SEQ ID NOs: 1676-1851, SEQ ID NOs: 2028-2529, SEQ ID NOs: 3046-3221, SEQ ID NOs: 3398-3899, and SEQ ID NOs: 4402-4530, or SEQ ID NOs: 4531-5794, SEQ ID NOs: 7059-8322, or SEQ ID NOs: 9596-9603, or a pharmaceutically acceptable salt thereof;
wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or
wherein at least one (i.e., one or more) nucleoside is substituted with a component selected from the group consisting of a 2′-O-(2-methoxyethyl) nucleoside, a 2′-O-methyl nucleoside, a 2′-O—(N-methylacetamide) nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA)
optionally, wherein the oligonucleotide further comprises a spacer.

164. The method of any one of claims 157-163, wherein nucleoside linkages that link a base at position 3 or position 4 of the oligonucleotide are phosphodiester linkages.

165. The method of claim 164, wherein only one nucleoside linkage that links a base at position 3 or position 4 of the oligonucleotide is a phosphodiester linkage.

166. The method of any one of claims 157-163, wherein nucleoside linkages that link bases at both position 3 and position 4 of the oligonucleotide are phosphodiester linkages.

167. The method of any one of claims 157-163, wherein one or more bases immediately preceding a spacer in the oligonucleotide are linked through phosphodiester bonds.

168. The method of claim 167, wherein only the base immediately preceding the spacer in the oligonucleotide is linked to the spacer through a phosphodiester bond.

169. The method of claim 168, wherein the base immediately preceding the spacer in the oligonucleotide is further linked to a further preceding base through a phosphodiester bond.

170. The method of claim 168, wherein the oligonucleotide comprises a second spacer, wherein a base immediately preceding the second spacer is linked to a further preceding base through a phosphodiester bond.

171. The method of any one of claims 157-163, wherein one or more bases immediately succeeding a spacer in the oligonucleotide are linked through phosphodiester bonds.

172. The method of claim 171, wherein only the base immediately succeeding the spacer in the oligonucleotide is linked to the spacer through a phosphodiester bond.

173. The method of any one of claims 157-163, wherein two bases immediately preceding the spacer in the oligonucleotide are linked through phosphodiester bonds.

174. The method of any one of claims 157-163, wherein one or more bases immediately preceding a spacer in the oligonucleotide are linked through phosphodiester bonds and wherein one or more bases immediately succeeding the spacer in the oligonucleotide are linked through phosphodiester bonds.

175. The method of claim 174, wherein one base immediately preceding the spacer and one base immediately succeeding the spacer are linked through phosphodiester bonds.

176. The method of claim 174 or 175, wherein the oligonucleotide includes a second spacer, and wherein one or more bases immediately preceding the second spacer in the oligonucleotide are linked through phosphodiester bonds and wherein one or more bases immediately succeeding the second spacer in the oligonucleotide are linked through phosphodiester bonds.

177. The method of claim 176, wherein one base immediately preceding the second spacer and one base immediately succeeding the second spacer are linked through phosphodiester bonds.

178. The method of any one of claims 157-163, wherein the oligonucleotide comprises a range of bases that are linked through phosphodiester bonds, the range of bases comprising at least two bases.

179. The method of any one of claims 157-163, wherein the oligonucleotide comprises a range of bases that are linked through phosphodiester bonds, the range of bases comprising at least five bases.

180. The method of claim 178 or 179, wherein the oligonucleotide comprises two or more spacers, and wherein the range of bases are positioned between the at least two spacers.

181. The method of any of claims 164-180, wherein the oligonucleotide is any one of a 19mer, 21mer, 23mer, or 25mer.

182. The method of any one of claims 157-163, wherein at least one (i.e., one or more) internucleoside linkage of the oligonucleotide is a phosphorothioate linkage.

183. The method of any one of claims 157-163, wherein all internucleoside linkages of the oligonucleotide are phosphorothioate linkages.

184. An oligonucleotide and a pharmaceutically acceptable excipient, the oligonucleotide comprising a sequence that is at least 85% complementary to an equal length portion of any one of SEQ ID NO: 1339 or SEQ ID NO: 1341, SEQ ID NO: 3032-3043, SEQ ID NO: 9587-9595, or SEQ ID NO: 9698-9707, a sequence having 90% identity thereof, or to a 15 to 50 contiguous nucleobase portion thereof, optionally wherein the oligonucleotide comprises a spacer and wherein the oligonucleotide is capable of increasing, restoring, or stabilizing expression of one or more of STMN2, KCNQ2, UNC13A, or SMN2 mRNA capable of translation of a functional protein in a cell or a human patient of an immune-mediated demyelinating disease, and wherein the level of increase, restoration, or stabilization of expression and/or activity and/or function is sufficient for use of the oligonucleotide as a medicament for the treatment of the immune-mediated demyelinating disease.

185. The method of any one of claims 121-141 or 144-183, the pharmaceutical composition of claim 142 or 143, or the oligonucleotide of any one of claims 1-120 or 184, wherein the oligonucleotide comprises one or more chiral centers and/or double bonds.

186. The method of any one of claims 121-141, 144-183, or 185, the pharmaceutical composition of claim 142, 143, or 185, or the oligonucleotide of any one of claims 1-120 or 184-185, wherein the oligonucleotide exist as stereoisomers selected from geometric isomers, enantiomers, and diastereomers.

187. A method of treating a neurological disease and/or a neuropathy in a patient in need thereof, the method comprising administering to a patient in need thereof a therapeutically effective amount of a pharmaceutical composition of claim 142 or 143, in combination with a second therapeutic agent.

188. The method of claim 187, wherein the second therapeutic agent is selected from Riluzole (Rilutek), PrimeC, Edaravone (Radicava), rivastigmine, donepezil, galantamine, selective serotonin reuptake inhibitor, antipsychotic agents, cholinesterase inhibitors, memantine, benzodiazepine antianxiety drugs, AMX0035 (ELYBRIO), ZILUCOPLAN (RA101495), pridopidine, dual AON intrathecal administration (e.g., BIIB067, BIIB078, and BIIB05), BIIB100, levodopa/carbidopa, dopaminergic agents (e.g., ropinirole, pramipexole, rotigotine), medroxyprogesetrone, KCNQ2/KCNQ3 openers (e.g., retigabine, XEN1101, or QRL-101), bioactive scaffolds, anticonvulsants and psychostimulant agents, a therapy (e.g., selected from breathing care, physical therapy, occupational therapy, speech therapy, nutritional support), deep brain stimulation, levodopa and carbidopa (duopa, rytary, Sinemet, inbrija), istradefylline (nourianz), safinamide (xadago), pramipexole (Mirapex), rotigotine (neupro), ropinirole (requip), amantadine (gocovri, Symmetrel, osmolex), benztropine (Cogentin), trihexyphenidyl (artane), selegiline (eldepryl, zelapar), rasagiline, entacapone (comtan), opicapone (ongentys), tolcapone (tasmar), apomorphine (apokyn, kynmobi), exenatide, lingzhi, BIIB054, BIIB094, Caffeine, sarizotan, embryonic dopamine cell implantation, aducanamab (Aduhlem), memantine (Namenda), Donepezil (Aricept), Rivastigmine (Exelon), Galantamine (razadyne), Namzeric, Suvorexant (belsomra), lecanemab, olanzapine (Zyprexa), quetiapine (Seroquel), SSRIs (citalopram (Cipramil), dapoxetine (Priligy), escitalopram (Cipralex), fluoxetine (Prozac or Oxactin), fluvoxamine (Faverin), paroxetine (Seroxat), sertraline (Lustral), vortioxetine (Brintellix)), divalproex sodium (Depakote), carbamazepine (Tegretol), medroxyprogestrone, Brivaracetam (briviact), cannabidiol (epidiolex), carbamazepine (carbatrol, Tegretol), cenobamate (xcopri), diazepam (valium), lorazepam (Ativan), clonazepam (klonopin), eslicarbazepine (aptiom), ethosuximide (zarontin), felbamate (felbatol), fenfluramine (fintepla), lacosamide (VIMPAT), lamotrigine (Lamictal), levetiracetam (Keppra), oxcarbazepine (oxtellar xr, Trileptal), perampanel (fycompa), phenobarbital, phenytoin (dilantin), pregabalin (lyrica), tiagabine (gabitril), topiramate (topamax), valproate (depakene, depakote), and/or zonisamide (zonegran), for treating said neurologic disease.

189. A method of treating a neurological disease and/or a neuropathy in a patient in need thereof, the method comprising administering to a patient in need thereof a therapeutically effective amount of a pharmaceutical composition of claim 142 or 143, wherein at least one nucleoside linkage of the oligonucleotide is a non-natural linkage, optionally wherein the oligonucleotide comprises a spacer, and wherein the oligonucleotide further comprises a targeting or conjugate moiety selected from cholesterol, lipoic acid, panthothenic acid, polyethylene glycol, and an antibody for crossing the blood brain barrier.

190. The method of any one of claims 121-141, 144-183, or 185-189, wherein the spacer is a nucleoside-replacement group comprising a non-sugar substitute that is incapable of linking to a nucleotide base.

191. The method of claim 190, wherein the spacer is located between positions 10 and 15 of the oligonucleotide.

192. The method of claim 190, wherein the spacer is located between positions 7 and 11 of the oligonucleotide.

193. The method of claim 190 or 192, wherein the oligonucleotide further comprises a second spacer, wherein the second spacer is located between positions 14 and 22 of the oligonucleotide.

194. The method of claim 193, wherein the spacer and the second spacer are separated by at least 5 nucleobases, at least 6 nucleobases, or at least 7 nucleobases in the oligonucleotide.

195. The method of claim 193 or 194, wherein the spacer is located between positions 7 and 9 of the oligonucleotide, and wherein the second spacer is located between positions 15 and 18 of the oligonucleotide.

196. The method of any one of claims 193-195, wherein the spacer is located at position 8 of the oligonucleotide, and wherein the second spacer is located at position 16 of the oligonucleotide.

197. The method of any one of claims 193-196, wherein the oligonucleotide further comprises a third spacer, wherein the third spacer is located between positions 21 and 24 of the oligonucleotide.

198. The method of claim 190, wherein the spacer is located between positions 2 and 5 of the oligonucleotide.

199. The method of claim 198, wherein the oligonucleotide further comprises a second spacer, wherein the second spacer is located between positions 8 and 12 of the oligonucleotide.

200. The method of claim 199, wherein the oligonucleotide further comprises a third spacer, wherein the third spacer is located between positions 18 and 22 of the oligonucleotide.

201. The method of claim 190, wherein the oligonucleotide further comprises a second spacer and a third spacer, wherein the three spacers are located at positions in the oligonucleotide such that each segment of the oligonucleotide has at most 7 linked nucleosides.

202. The method of claim 201, wherein at least two of the three spacers are adjacent to a guanine nucleobase.

203. The method of claim 202, wherein each of the at least two of the three spacers immediately precede a guanine nucleobase.

204. The method of any one of claims 190-203, wherein each of the first, second or third spacers is a nucleoside-replacement group comprising a non-sugar substitute wherein the non-sugar substitute does not contain a ketone, aldehyde, ketal, hemiketal, acetal, hemiacetal, aminal or hemiaminal moiety and is incapable of forming a covalent bond with a nucleotide base.

205. The method of any one of claims 190-203, wherein each of the first, second or third spacers is independently represented by Formula (X), wherein: 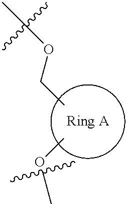
Ring A is an optionally substituted 4-8 member monocyclic cycloalkyl group or a 4-8 member monocyclic heterocyclyl group, wherein the heterocyclyl group contains 1 or 2 heteroatoms selected from O, S and N, provided that A is not capable of forming a covalent bond to a nucleobase; and
the 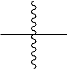
symbol represents the point of connection to an internucleoside linkage.

206. The method of claim 205, wherein each of the first, second or third spacers is independently represented by Formula (Xa), wherein:

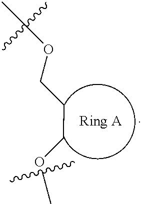

207. The method of claim 205 or 206, wherein ring A is an optionally substituted 4-8 member monocyclic cycloalkyl group selected from cyclobutyl, cyclopentyl, cyclohexyl, cycloheptyl and cyclooctyl; or a 4-8 member monocyclic heterocyclyl group, selected from oxetanyl, tetrahydrofuranyl, tetrahydropyranyl, 1,4-dioxanyl, pyrolidinyl, piperidinyl, piperazinyl, morpholinyl and azepanyl.

208. The method of claim 207, wherein ring A is tetrahydrofuranyl.

209. The method of claim 207, wherein ring A is tetrahydropyranyl.

210. The method of any one of claims 190-203 wherein each of the first, second or third spacers is independently represented by Formula (I), wherein: 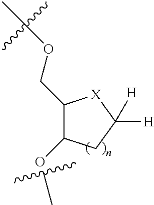
X is selected from —CH₂— and —O—; and
n is 0, 1, 2 or 3.

211. The method of any one of claims 190-203, wherein each of the first, second or third spacers is independently represented by Formula (I′), wherein:

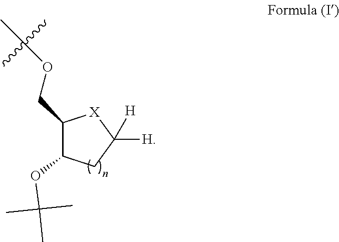

212. The method of any one of claims 190-203, wherein each of the first, second or third spacers is independently represented by Formula (Ia), wherein:

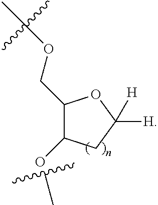

213. The method of any one of claims 190-203, wherein each of the first, second or third spacers is independently represented by Formula (Ia′), wherein:

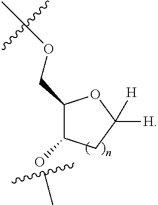

214. The method of any one of claims 190-203, wherein each of the first, second or third spacers is independently represented by Formula II, wherein: 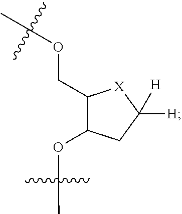
and
X is selected from —CH₂— and —O—.

215. The method of any one of claims 190-203, wherein each of the first, second or third spacers is independently represented by Formula II′, wherein: 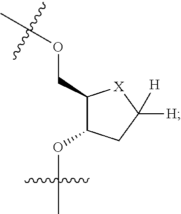
and
X is selected from —CH₂— and —O—.

216. The method of any one of claims 190-203, wherein each of the first, second or third spacers is independently represented by Formula (Ha), wherein:

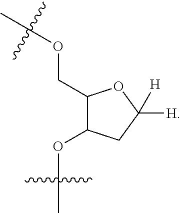

217. The method of any one of claims 190-203, wherein each of the first, second or third spacers is independently represented by Formula (Iia′), wherein:

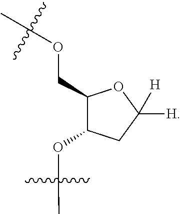

218. The method of any one of claims 190-203, wherein each of the first, second or third spacers is independently represented by Formula (IIi), wherein: 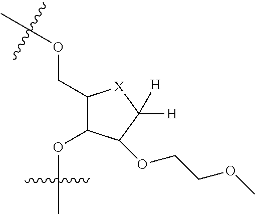
X is selected from —CH₂— and —O—.

219. The method of any one of claims 190-203, wherein each of the first, second or third spacers is independently represented by Formula (IIi′), wherein: 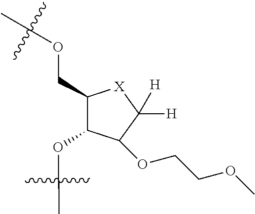
X is selected from —CH₂— and —O—.

220. The method of any one of claims 190-203, wherein each of the first, second or third spacers is independently represented by Formula (IIib), wherein:

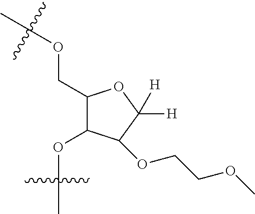 [SMILES: *OCC1OCC(OCCOC)C1O*]

221. The method of any one of claims 190-203, wherein each of the first, second or third spacers is independently represented by Formula (IIib′), wherein:

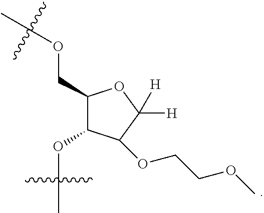 [SMILES: *OC[C@H]1OCC(OCCOC)[C@@H]1O*]

222. The method of any one of claims 190-203, wherein each of the first, second or third spacers is independently represented by Formula III, wherein: 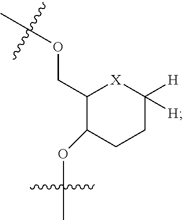
and
X is selected from —CH₂— and —O—.

223. The method of any one of claims 190-203, wherein each of the first, second or third spacers is independently represented by Formula III′, wherein: 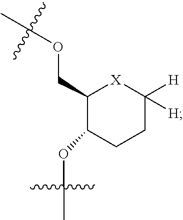
and
X is selected from —CH₂— and —O—.

224. The method of any one of claims 190-203, wherein each of the first, second or third spacers is independently represented by Formula (IIIa), wherein:

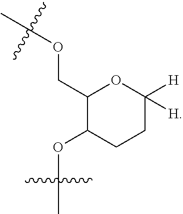

225. The method of any one of claims 190-203, wherein each of the first, second or third spacers is independently represented by Formula (IIIa′), wherein:

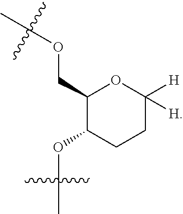

226. The method of any one of claims 190-225, wherein the oligonucleotide comprising the spacer has a GC content of at least 10%.

227. The method of any one of claims 190-226, wherein the oligonucleotide comprising the spacer has a GC content of at least 20%.

228. The method of any one of claims 190-227, wherein the oligonucleotide comprising the spacer has a GC content of at least 25%.

229. The method of any one of claims 190-228, wherein the oligonucleotide comprising the spacer has a GC content of at least 30%.

230. The method of any one of claims 190-229, wherein the oligonucleotide comprising the spacer has a GC content of at least 40%.

231. The method of any one of claims 190-230, wherein the oligonucleotide comprising the spacer has a GC content of at least 50%.

232. A method for synthesizing an AON with a spacer, the method comprising:
obtaining compound A represented by the formula of 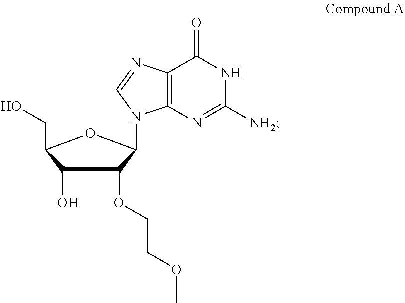
performing a benzoyl protection reaction of compound A to generate compound B represented by the formula of 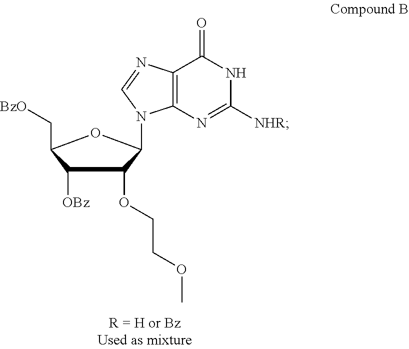
performing reductive nucleobase cleavage of compound B to generate compound C represented by the formula of 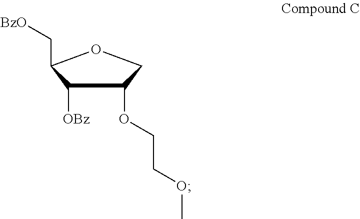
performing a saponification reaction of compound C to generate compound D represented by the formula of 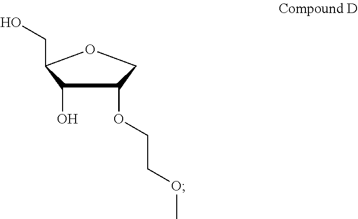
performing a protection reaction of compound D to generate compound E represented by the formula of 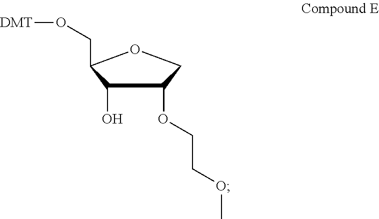
and
performing a phosphoramidite installation reaction of compound E to generate compound F represented by the formula of

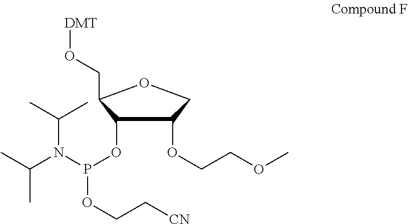

233. A composition comprising:
compound F having formula [chemical structure]
